(19) United States
(12) Patent Application Publication
(10) Pub. No.: US 2026/0217708 A1
(43) Pub. Date: Jul. 30, 2026

(54) FUSED BENZAZEPINE DERIVATIVES FOR USE IN THE TREATMENT OF CANCER AND EPILEPSY

(71) Applicant: UCB BIOPHARMA SRL, Brussels (BE)

(72) Inventors: Ali ATES, Brussels (BE); Christopher Edward BLUNT, Nottingham Nottinghamshire (GB); Pierre BURSSENS, Brussels (BE); Dimitri Francois Joseph CAPUTO, Nottingham Nottinghamshire (GB); Mauro CORTIGIANI, Nottingham Nottinghamshire (GB); Sabine DEFAYS, Brussels (BE); Maria Teresa DE HARO GARCIA, Brussels (BE); Christel DELAUNOY, Brussels (BE); Simon Fraser HUNT, Nottingham Nottinghamshire (GB); Jérôme JACQ, Brussels (BE); Alexandre Emile René JOLIBOIS, Nottingham Nottinghamshire (GB); Jean KEYAERTS, Brussels (BE); Marie Adolphine C. LEDECQ, Brussels (BE); Baptiste MANTEAU, Brussels (BE); Laurent PROVINS, Brussels (BE); Dominique Louis L. SWINNEN, Brussels (BE); Riccardo ZAMBELLONI, Nottingham Nottinghamshire (GB); Ricardo ZANALETTI, Nottingham Nottinghamshire (GB)

(21) Appl. No.: 19/151,852

(22) PCT Filed: Feb. 1, 2023
(86) PCT No.: PCT/EP2023/052386

§ 371 Date: Jul. 29, 2025

(51) Int. Cl. C07D 471/04; A61K 31/551; C07B 59/00; C07D 471/14; C07D 487/04; C07D 491/052; C07D 491/147; C07D 495/14; C07D 513/04; C07D 519/00

(52) U.S. Cl. CPC C07D 471/04; A61K 31/551; C07B 59/002; C07D 471/14; C07D 487/04; C07D 491/052; C07D 491/147; C07D 495/14; C07D 513/04; C07D 519/00; C07B 2200/05

(57) ABSTRACT

The present invention relates to a compound of formula (I) or a pharmaceutically acceptable salt thereof; Formula (I) which is useful for the treatment of diseases and/or disorders in which System Xc⁻ plays a role.

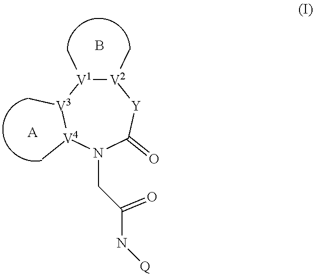

The invention relates to substituted 7-membered cyclic amides or ureas derivatives and their use in therapy.

In particular, the present invention relates to pharmacologically active substituted 7-membered cyclic amides or ureas derivatives and analogs thereof.

More particularly, the present invention relates to (hetero)aryl-acetamide of 7-membered cyclic amides or ureas derivatives and analogs thereof. The compounds according to the present invention modulate the System Xc⁻ cystine/glutamate antiporter and accordingly are of benefit as pharmaceutical agents for the treatment of diseases in which System Xc⁻ cystine/glutamate antiporter plays a role.

### BACKGROUND OF THE INVENTION

System Xc⁻, also known as the cystine/glutamate antiporter, is an amino acid transporter that mediates the extrusion of intracellular L-glutamate and the uptake of extracellular L-cystine, which undergoes intracellular reduction to L-cysteine. The influx of L-cystine serves as a rate-limiting step in providing L-cysteine, which is required for the synthesis of glutathione (GSH), the principal antioxidant in cells. L-Glutamate extruded by System Xc⁻ can serve as neurotransmitter. System Xc⁻ is a complex formed of two proteins, xCT (coded by the SLC7A11 gene) also called the light chain, and CD98 hc (SLC3A2) also called heavy chain or 4F2 hc. System Xc⁻ is expressed predominantly in the brain, in some glial cells such as astrocytes and microglia, and in non-CNS cells such as endothelial cells, fibroblasts, macrophages and hepatocytes.

In many different cancer types, system Xc⁻ is overexpressed compared to normal tissue. Those include, but are not limited to glioma (particularly glioblastoma) (Takeuchi et al. Neurosurgery (2013), 72, 33-41), colon carcinoma, colorectal carcinoma (Sugano et al. Anticancer Res (2015), 35, 677-682), non-small cell lung carcinoma (adenocarcinomas and squamous cell carcinomas) and other lung cancer types (Ji et al. Oncogene (2018), 37, 5007-5019), esophageal carcinoma, cancer stem cells in triple negative breast cancer (Conti et al., Cancer Immunol Res (2020), 8, 1039-105) and hepatocellular carcinoma (Kavanaugh et al., Mol Imaging Biol (2016) 18, 924-934). High system Xc⁻ expression is associated with poor prognosis in several cancers including but not limited to colon carcinoma (Lim et al., Proc Natl Acad Sci USA (2019), 116, 9433-9442), adrenocortical carcinoma, kidney carcinoma (Wang et al., Oncotarget 2016, 7, 29901-29915), hepatocellular carcinoma (Kinoshita et al., Oncolumn Rep (2013), 29, 685-689), mesothelioma, lung carcinoma (Ji et al. Oncogene (2018), 37, 5007-5019), sarcoma, uveal melanoma and gastric cancer (Luo et al., Oncotarget 8, (2017), 112530-112549). In pancreatic ductal adenocarcinoma, a particular form of pancreatic carcinoma, stroma cells heavily rely on cysteine to prevent ferroptotic cell death and depletion of SLC7A11 in cancer-associated fibroblasts prevents orthotopic pancreatic tumor formation (Sharbeen et al., Cancer Res (2021); DOI: 10.1158/0008-5472.CAN-20-2496). In other cancers, System Xc⁻ plays a crucial role in tumorigenesis, because down regulation of SLC7A11 (the light chain of system Xc⁻) in cancer cells decreases cancer cell proliferation, tumor progression and invasion (Badgley et al., Science (2020), 368, 85-89; Ede et al., Haematologica (2018), 103, 1496-1501; Hu et al., J Clin Invest (2020), 130, 1752-1766; Lei et al., Cell Res (2020), 30, 146-162; Lin et al., Am J Cancer Res (2020), 10, 3106-3126).

High system Xc⁻ levels also confer to the cell increased capacity for the anti oxidant GSH synthesis, defense against reactive oxygen species (ROS) and tumor growth (Liu et al., Mol Ther (2020), 28, 2358-2366).

In addition, SLC7A11, cystine and cysteine have been described to play a role in radiotherapy resistance and in multidrug resistance in several cancer types (Horibe et al., Biochem Biophys Res Commun (2018), 507, 426-432; Koppula et al., Cell Res (2020), 30, 146-162).

Thus, inhibiting or blocking System Xc⁻ may be useful for the treatment of certain cancers where System Xc⁻ plays a role.

Blocking System Xc⁻ can also synergize with other therapies targeting tumor growth. For example, inhibition of System Xc⁻ preventing cancer stem cell metastasis, together with chemotherapy treatment blocking tumor growth (induced by oncogenes such as HER2, p53, Kras and others), leads to additional therapeutic effects in breast, esophageal and other cancer cell lines and models (Conti et al., Cancer Immunol Res (2020), 8, 1039-53; Liu et al., Nat Commun (2017), 8, 14844).

In several cancer cells, toxic lipid peroxidation induced by inhibition of System Xc, when combined to conventional cancer therapy can have a synergistic effect, lead to cancer cell death and overcome resistance to this conventional cancer therapy (Lin et al., Am J Cancer Res (2020), 10, 3106-3126; Zhu et al. Cancer Res (2021) 77(8), 2064-2077).

Therefore, molecules inhibiting system Xc⁻ could be used alone or in combination therapy, with molecules or treatments targeting other mechanisms and pathways involved in cancer biology, and thereby help overcome drug resistance in current cancer treatments or enhance the effect of certain existing treatments. Glutamate release due to upregulation of system Xc⁻ in cancer cells also affects tumorigenesis, and inhibition of glutamate release is correlated with a decrease in proliferation not only in brain tumors, but also in non-brain carcinoma ((Savaskan et al., Nature Medicine (2008), 14, 629; Lewerenz et al., Antioxid Redox Signal (2013), 18, 522-555; Corsi et al., Int J Mol Sci (2019), 20).

System Xc⁻ induced efflux of L-Glutamate into the extracellular space can contribute to excitatory signaling and to excitotoxicity, leading to seizures, neuronal death, and other brain pathologies through activation of postsynaptic glutamate receptors on neurons. Conversely, mice lacking system Xc⁻ have decreased brain glutamate receptors and demonstrate decreased or delayed epileptogenesis (Leclercq et al., Epilepsia (2019), 60, 1412-1423). Glioblastoma cells expressing elevated System Xc⁻ levels release high levels of glutamate, which activates glutamate receptors on neighboring neurons, and induces neuronal hyperactivity and seizures (Marcus et al., J. Neurooncol. (2010), 97, 11-23; Robert et al., (2015), Sci Transl Med 7, 289ra286).

Inhibiting system Xc⁻ function or expression could therefore prevent glutamate-induced seizures and neuronal death in glioma-associated epilepsy patients and in other epilepsy syndromes presenting high System Xc⁻ levels, such as focal cortical dysplasia and tuberous sclerosis (Arena et al., (2019), Brain Pathol 29, 351-365).

International patent application WO 2015/196086 relates to compounds that are stated to be inhibitors of System Xc⁻.

Sulfasalazine is approved for the treatment of disorders, including rheumatoid arthritis, ulcerative colitis, and Crohn's disease. It has been demonstrated to be a non-selective inhibitor of the System Xc⁻ antiporter; however, due to its poor brain exposure, its uses are limited to peripheral indications. In addition, due to its low potency on human System Xc⁻ function, its efficacy in peripheral indications is limited.

There is therefore a need to design new agents that inhibit the System Xc⁻ antiporter, which agents have improved properties and can be used for the treatment of certain cancers, or epilepsy syndromes where system Xc⁻ plays a role.

In order to accelerate the identification of suitable inhibitors of the System Xc⁻ antiporter, there is also a need to develop pharmacological tools that can be used in the development of new biological tests.

### SUMMARY OF THE INVENTION

In a first aspect, the present invention provides a compound of formula (I) or a pharmaceutically acceptable salt thereof,

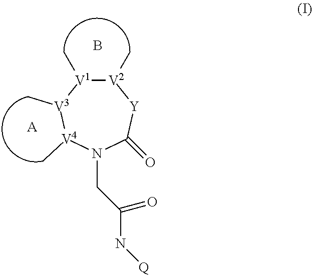

Wherein
Y represents N—R^{a} or CR^{1a}R^{1b};
R^{a} represents hydrogen or C₁₋₄ alkyl;
R^{1a} and R^{1b} represent independently hydrogen, hydroxy, halogen; or C₁₋₄ alkoxy or C₁₋₄ alkyl, either of which groups may be optionally substituted with one or more substituents; and
A, together with the points of attachment to the remainder of the molecule, V³ and V⁴, represents an optionally substituted aryl or heteroaryl selected from the groups represented by A¹, A², A³, A⁴, A⁵, A⁶ and A⁷:

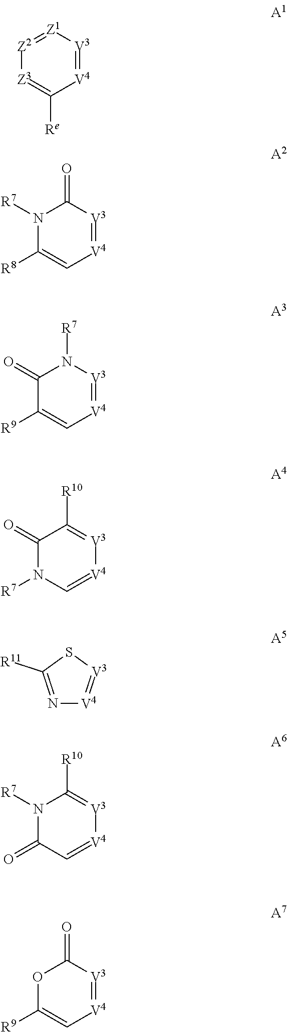

Wherein
V³ and V⁴ represent independently C;
Z¹ represents N or C—R⁴
Z² represents N or C—R⁵;
Z³ represents N or C—R⁶;
R³ represents hydrogen or halogen;
R⁴ represents hydrogen, halogen, hydroxy, cyano or amino; or C₁₋₄ alkyl, C₃₋₇ cycloalkyl or C₁₋₄ alkoxy, any of which groups may be optionally substituted by one or more substituents;
R⁵ represents hydrogen, halogen or cyano; or C₁₋₄ alkyl or C₁₋₄ alkoxy, either of which groups may be optionally substituted by one or more substituents;
R⁶ represents hydrogen, halogen, or cyano; or C₁₋₄ alkoxy, C₁₋₄ alkylamino, C₁₋₄ alkyl, C₃₋₇ heterocycloalkyl or —O—(C₃₋₇ heterocycloalkyl), any of which groups may be optionally substituted by one or more substituents;
R⁷ represents hydrogen; or C₁₋₄ alkyl or C₃₋₇ cycloalkyl, either of which groups may be optionally substituted by one or more substituents;
R⁸, R⁹ and R¹⁰ independently represent hydrogen or halogen; or C₁₋₄ alkyl, C₃₋₇ cycloalkyl or C₁₋₄ alkoxy, any of which groups may be optionally substituted by one or more substituents;
R¹¹ represents —NR^{c}—(CO)—R^{b};
R^{b} and R^{c} represent independently C₁₋₄ alkyl; and
B, together with the points of attachment to the remainder of the molecule, V¹ and V², represents an optionally substituted aryl or heteroaryl selected from the groups represented by B¹, B², B³, B⁴, B⁵, B⁶, B⁷ and B⁸:

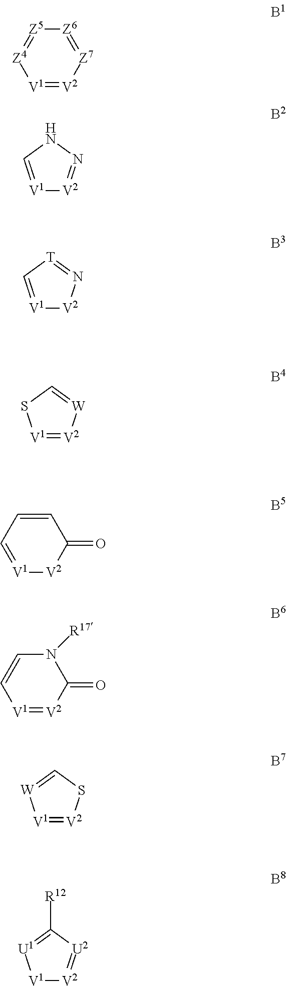

Wherein
V¹ represents C for B¹, B², B³, B⁴, B⁵, B⁶ and B⁷ and represents N for B⁸;
V² represents C for B¹, B², B⁴, B⁶, B⁷, and B⁸ and represents N for B³ and B⁵;
W, U¹ and U² represent independently N or C—H;
Z⁴ represents N or C—R¹³;
Z⁵ represents N or C—R¹⁴;
Z⁶ represents N or C—R¹⁵;
Z⁷ represents N or C—R¹⁶;
T represents N or C—R¹⁷;
R¹² represents hydrogen;
R¹³, R¹⁴, R¹⁵, R¹⁶ represent independently hydrogen, halogen, or cyano; or C₁₋₄ alkyl or C₁₋₄ alkoxy, either of these groups which may be optionally substituted by one or more substituents; and
R¹⁷ represents hydrogen, halogen or C₁₋₄ alkyl;
R¹⁷′ represents hydrogen or C₁₋₄ alkyl; and
Q represents a ring selected from the groups represented by Q¹ and Q²:

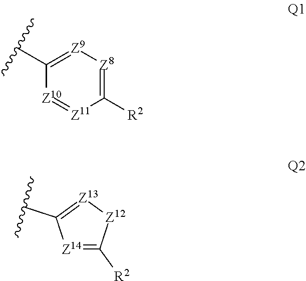

Wherein
Z⁸ represents N or C—R³;
Z⁹ represents C—R¹⁸;
Z¹⁰ represents N or C—R¹⁹;
Z¹¹ represents C—R²⁰;
Z¹² represents S, O, N—H or CR²¹R²²;
Z¹³ represents N or C—R²³;
Z¹⁴ represents N or C—R²⁴;
R² represents halogen, or cyano; or C₁₋₄ alkyl, C₃₋₇ cycloalkyl or C₁₋₄ alkoxy, any of which groups may be optionally substituted by one or more substituents;
R³ represents hydrogen, halogen or cyano; or C₁₋₄ alkyl, which group may be optionally substituted by one or more substituents; or
R² and R³ together with the group to which they are attached form a cycloalkyl, heterocyclyl, aryl or heteroaryl, which group is optionally substituted with one or more substituents;
R¹⁸ represents hydrogen or halogen; or C₁₋₄ alkyl which may be optionally substituted by one or more substituents; or
R¹⁹, R²⁰, R²¹, R²², R²³ and R²⁴ represent independently hydrogen or halogen; or C₁₋₄ alkyl which may be optionally substituted by one or more substituents.

In a second aspect, the present invention provides a compound of Formula (I), or a pharmaceutically acceptable salt thereof, for use in therapy.

In a third aspect, the present invention provides compound of Formula (I), or a pharmaceutically acceptable sale thereof, useful for the treatment of disorders for which system Xc-hydroxyl/glutamate antiporter plays a role.

In particular, the present invention provides compounds of formula (I) which may be useful for the treatment of cancers, or epilepsy syndromes where system Xc⁻ plays a role.

Furthermore, the present invention provides compounds of formula (I) which may be useful to overcome cancer treatment resistance.

In a fourth aspect, the present invention provides a pharmaceutical composition comprising, as an active ingredient, a compound of Formula (I), or a pharmaceutically acceptable salt thereof, in combination with one or more pharmaceutically acceptable carriers, diluents, or excipients.

In a fifth aspect, the present invention provides synthetic intermediates of Formula (II) useful for the chemical synthesis of compounds of formula (I).

### DETAILED DESCRIPTION OF THE INVENTION

The term “C₁₋₄ alkyl” as used herein refers to straight or branched, monovalent, saturated aliphatic hydrocarbon chains of 1 to 4 carbon atoms. Illustrative C₁₋₄ alkyl according to the present invention are methyl and ethyl.

The term “C₁₋₄ alkoxy” represents a group of formula —O—R where R is a “C₁₋₄ alkyl,” as described herein, wherein the C₁-C₄ alkoxy group is connected to the parent structure via the oxygen atom. Suitable alkoxy groups according to the present invention include methoxy.

The term “C₃₋₇ cycloalkyl” as used herein refers to monovalent groups of 3 to 7 carbon atoms derived from a saturated monocyclic hydrocarbon. Illustrative C₃₋₇ cycloalkyl groups include cyclopropyl.

The term “C₃₋₇ heterocycloalkyl” as used herein refers to saturated monocyclic and bicyclic rings containing 3 to 7 carbon atoms and at least one heteroatom selected from oxygen, sulphur and nitrogen. Suitable C₃₋₇ heterocycloalkyl according to the present invention include azetidinyl, piperazinyl, morpholino, pyrrolydinyl, azaspirohexanyl, azaspiroheptanyl, azabicyclohexanyl, azabicycloheptanyl, oxa-azaspiroheptanyl and oxa-azaspirooctanyl.

In one particular embodiment in which the compounds of the present invention are compounds of formula (I), the term “C₃₋₇ heterocycloalkyl” refers to saturated monocyclic rings containing 3 to 7 carbon atoms and at least one heteroatom selected from oxygen, sulphur and nitrogen. Suitable C₃₋₇ heterocycloalkyl according to this particular embodiment include azetidinyl and piperazinyl.

The terms “Halo,” “halogen,” and “halide” are used indifferently and represent a chloro, fluoro, bromo, or iodo atom. Suitable examples of halogens according to the present invention include chloro and fluoro.

The term “amino” as used herein refers to —NH₂ if it is a primary amine group, —NH if it is a secondary amine group or —N— if it is a tertiary amine group, wherein the nitrogen will be linked to the parent molecule. For example, “amino” used in the term C₁₋₄ alkylamino, refers to an NH substituted by a C₁₋₄ alkyl and wherein the nitrogen is linked to the parent molecule.

The term “aryl” as used herein represents an unsaturated carbocyclic group of from 6 to 14 carbon atoms having a single ring (e.g. phenyl) or multiple condensed rings (e.g. naphthyl).

When the compounds of the present invention are compounds of formula (I), the term “aryl” as used herein represents an unsaturated heteroaromatic or carbocyclic group of from 6 to 14 carbon atoms having a single ring (e.g. phenyl) or multiple condensed rings (e.g. naphthyl).

In one aspect where the compounds of the present invention are compounds of formula (I), the term “aryl” as used herein represents an unsaturated carbocyclic group of from 6 to 14 carbon atoms having a single ring (e.g. phenyl) or multiple condensed rings (e.g. naphthyl).

For the avoidance of doubt, when reference is made to the compounds of formula (I) this also embraces compounds of formulae (IA), (IA-a), (IB), (IB-a), (IC), (IC-a), (ID), (ID-a), (IE), (IE-a), (IF), (IG), (IJ), (IK), (IL) and (IM).

The term “heteroaryl” as used herein represents aromatic carbocyclic groups of from 5 to 14 carbon atoms, having a single ring or multiple condensed rings, wherein one or more of the said carbon atoms have been replaced by one or more heteroatoms selected from oxygen, sulphur and nitrogen.

Where any of the groups in the compounds of formula (I) above is stated to be optionally substituted, this group may be unsubstituted, or substituted by one or more substituents. Typically, such groups will be unsubstituted, or substituted by one, two or three substituents. In one embodiment, such groups are unsubstituted. Suitable substituents for each of the groups present on compounds of formula (I) are further described here after in the present specification.

Formula (I) and the formulae depicted hereinafter are intended to represent all individual stereoisomers and all possible mixtures thereof, unless stated or shown otherwise.

Stereoisomers of compounds formula (I) include cis and trans isomers, optical isomers, diastereomers, geometric isomers, rotational isomers, atropisomers, and conformational isomers of the compounds of formula (I), including compounds exhibiting more than one type of isomerism; and mixtures thereof (such as racemates and diastereomeric pairs).

Compounds of Formula (I) and/or their intermediates may have at least one stereogenic center in their structure. This stereogenic center may be present in a R or a S configuration (referred to as aR or aS for atropisomers), said R and S (or aR and aS) notation is used in correspondence with the rules described in Pure Appl. Chem., 45 (1976) 11-30. The invention thus also relates to all stereoisomeric forms such as enantiomeric and diastereoisomeric forms of the compounds of Formula (I) or mixtures thereof (including all possible mixtures of stereoisomers). With respect to the present invention reference to a compound or compounds is intended to encompass that compound in each of its possible isomeric forms and mixtures thereof, unless the particular isomeric form is specifically referred to.

The carbon-carbon bonds of the compounds of formula (I) are depicted herein using a solid line (—), a solid wedge ( [symbol] ), or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of formula (I) may contain more than one asymmetric carbon atom. In those compounds, the use of a solid line to depict bonds to asymmetric carbon atoms is meant to indicate that all possible stereoisomers are meant to be included.

Some compounds of formula (I) may exist as single atropisomer or as mixture of atropisomers.

Atropiomers are stereoisomers arising because of hindered rotation about a single bond, where energy differences due to steric strain or other contributors create a barrier to rotation that is high enough to allow for isolation of individual conformers (see for example Bringmann G. et al. Atroposelective Synthesis of Axially Chiral Biaryl Compounds. Angewandte Chemie International Edition. (2005) 44 (34): 5384-5427).

Unlike compounds with classical chiral centers, which racemize via a bond breaking and making process, atropisomers racemize via an intramolecular dynamic process that only involves bond rotation. Depending on rotation barrier, one particular conformer of compounds of formula (I′) and formula (I) can be in equilibrium with another conformer and thus the composition of the confomers may change with time or condition to reach an equilibrium. The conformation of the compounds of formula (I′) and formula (I) can be represented with solid line (—) and/or with solid wedge ( [symbol] ). An example is displayed herebelow with a particular sub-group of compounds of Formula (I), which atropisomers are represented respectively by formula (IA-aa) and (IA-ab).

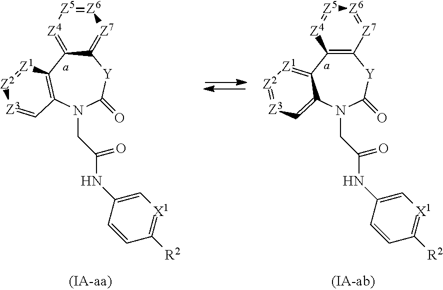

The use of the solid wedge [symbol] ) on the A and B rings is meant to indicate a conformation associated to the specific atropisomer (IA-aa) or (IA-ab).

Some of the compounds of formula (I) may exist in tautomeric forms. Such forms although not explicitly indicated in the above formula are intended to be included within the scope of the present invention. Examples of tautomers include keto (CH₂C═O)↔enol (CH═CHOH) tautomers or amide (NHC═O)↔hydroxyimine (N═COH) tautomers or 2-hydroxypyridine↔pyridinone. Formula (I) and the formulae depicted hereinafter are intended to represent all individual tautomers and all possible mixtures thereof, unless stated or shown otherwise.

It is also to be understood that each individual atom present in formula (I′), formula (I), or in the formula depicted hereinafter, may in fact be present in the form of any of its naturally occurring isotopes, with the most abundant isotope(s) being preferred.

Thus, by way of example, each individual hydrogen atom present in formula (I′), formula (I), or in the formula depicted hereinafter, may be present as a ¹H, ²H (deuterium) or ³H (tritium) atom, preferably ¹H or ²H. Similarly, by way of example, each individual carbon atom present in formula (I′), formula (I), or in the formulae depicted hereinafter, may be present as a ¹¹C, ¹²C, ¹³C or ¹⁴C atom, preferably ¹²C. Similarly, by way of example, each individual fluorine atom may be present as ¹⁸F or ¹⁹F.

Thus, the present invention, also includes within its scope, isotopically-labelled compounds of Formula (I).

Specific embodiments of compounds of formula (I) according to the present invention are described hereafter.

In one embodiment, Y represents N—R^{a}. Suitably, R^{a} represents methyl.

In another embodiment, Y represents CR^{1a}R^{1b}.

In a first embodiment, R^{1a} represents hydrogen. In a particular aspect of this embodiment, R^{1a} represents deuterium. In a second embodiment, R^{1a} represents hydroxyl. In a third embodiment, R^{1a} represents halogen. In one aspect of this embodiment R^{1a} represents fluoro. In a fourth embodiment, R^{1a} represents optionally substituted C₁₋₄ alkyl. In one aspect of this embodiment, R^{1a} represents optionally substituted methyl. In another aspect of this embodiment, R^{1a} represents deuterated methyl. In a fifth embodiment, R^{1a} represents optionally substituted C₁₋₄ alkoxy. In one aspect of this embodiment, R^{1a} represents optionally substituted methoxy.

In a first embodiment, R^{1b} represents hydrogen. In a particular aspect of this embodiment, R^{1b} represents deuterium. In a second embodiment, R^{1b} represents hydroxyl. In a third embodiment, R^{1b} represents halogen. In one aspect of this embodiment R^{1a} represents fluoro. In a fourth embodiment, R^{1b} represents optionally substituted C₁₋₄ alkyl. In one aspect of this embodiment, R^{1b} represents optionally substituted methyl. In another aspect of this embodiment, R^{1a} represents deuterated methyl. In a fifth embodiment, R^{1b} represents optionally substituted C₁₋₄ alkoxy. In one aspect of this embodiment, R^{1b} represents optionally substituted methoxy.

Typical examples of substituents on R^{1a} and R^{1b} include one, two or three substituents independently selected from C₁₋₄ alkoxy and hydroxyl. In one embodiment, typical examples of substituents on R^{1a} and R^{1b} include one or two substituents independently selected from C₁₋₄ alkoxy and hydroxyl. In a further embodiment, typical examples of substituents on R^{1a} and R^{1b} include one substituent selected from C₁₋₄ alkoxy and hydroxyl.

Particular examples of substituents on R^{1a} and R^{1b} include one, two or three substituents independently selected from methoxy and hydroxyl. In one embodiment, particular examples of substituents on R^{1a} and R^{1b} include one or two substituents independently selected from methoxy and hydroxyl. In a further embodiment, typical examples of substituents on R^{1a} and R^{1b} include one substituent selected from methoxy and hydroxyl.

Suitably, R^{1a} represents hydrogen, halogen, C₁₋₄ alkyl, C₁₋₄ alkyl substituted by hydroxyl, C₁₋₄ alkyl substituted by C₁₋₄ alkoxy, or C₁₋₄ alkoxy.

Illustratively, R^{1a} represents hydrogen, deuterium, fluoro, hydroxyl, methyl, deuterated methyl (—CD3), hydroxymethyl, methoxymethyl, or methoxy.

Suitably R^{1b} represents hydrogen or C₁₋₄ alkyl.

Illustratively R^{1b} represents hydrogen or methyl.

In a particular embodiment, R^{1a′} represents hydrogen, halogen, C₁₋₄ alkyl, C₁₋₄ alkyl substituted by hydroxyl, C₁₋₄ alkyl substituted by C₁₋₄ alkoxy, or C₁₋₄ alkoxy; and R^{1b} represents hydrogen.

In a first embodiment, A represents A1. In a second embodiment, A represents A². In a third embodiment, A represents A³. In a fourth embodiment, A represents A⁴. In a fifth embodiment, A represents A⁵. In a sixth embodiment, A represents A⁶. In a seventh embodiment, A represents A⁷.

In one embodiment, Z¹ represents N. In another embodiment, Z¹ represents C—R⁴.

In one embodiment, Z² represents N. In another embodiment, Z² represents C—R⁵.

In one embodiment, Z³ represents N. In another embodiment, Z³ represents C—R⁶.

In one embodiment, one or none of Z¹, Z² and Z³ represents N.

In a particular embodiment, Z¹ represents N, Z² represents C—R⁵, and Z³ represents C—R⁶.

In another particular embodiment, Z¹ represents C—R⁴, Z² represents N, and Z³ represents C—R^{6′}.

In a further particular embodiment, Z¹ represents C—R⁴, Z² represents C—R⁵, and Z³ represents N.

In yet a further particular embodiment, Z¹ represents C—R⁴, Z² represents C—R⁵, and Z³ represents C—R^{6′}.

In a first embodiment, Re represents hydrogen. In a second embodiment, Re represents halogen.

In one aspect of this embodiment, Re represents fluoro.

Suitably, R^{e} represents hydrogen or fluoro.

Typically, Re represents hydrogen.

In a first embodiment, R⁴ represents hydrogen. In a second embodiment, R⁴ represents halogen. In one aspect of this embodiment, R⁴ represents chloro. In another aspect of this embodiment, R⁴ represents fluoro. In a third embodiment, R⁴ represents hydroxyl. In a fourth embodiment, R⁴ represents cyano. In a fifth embodiment, R⁴ represents amino. In a sixth embodiment, R⁴ represents optionally substituted C₁₋₄ alkyl. In one aspect of this embodiment, R⁴ represents optionally substituted methyl. In another aspect of this embodiment, R⁴ represents optionally substituted ethyl. In a seventh embodiment, R⁴ represents optionally substituted C₃₋₇ cycloalkyl. In one aspect according to this embodiment, R⁴ represents optionally substituted cyclopropyl. In an eighth embodiment, R⁴ represents optionally substituted C₁₋₄ alkoxy. In one aspect according to this embodiment, R⁴ represents optionally substituted methoxy. In another aspect according to this embodiment, R⁴ represents optionally substituted ethoxy.

Typical examples of substituents on R⁴ include one, two or three groups selected from halogen, hydroxyl, and C₁₋₄ alkoxy.

Particular examples of substitutents on R⁴ include one, two or three groups selected from fluoro, hydroxyl, and methoxy.

Suitably, R⁴ represents hydrogen, halogen, cyano, amino, C₁₋₄ alkyl, C₁₋₄ alkoxy, C₁₋₄ alkyl substituted by one, two or three halogen or hydroxyl, or C₁₋₄ alkoxy substituted by one, two or three C₁₋₄ alkoxy.

Illustratively, R⁴ represents hydrogen, chloro, fluoro, cyano, hydroxyl, amino, methyl, ethyl, difluoromethyl, hydroxymethyl, cyclopropyl, methoxy, ethoxy, or (methoxy)ethoxy.

In a first embodiment, R⁵ represents hydrogen. In a second embodiment, R⁵ represents halogen. In one aspect of this embodiment, R⁵ represents fluoro. In a third embodiment, R⁵ represents cyano. In a fourth embodiment, R⁵ represents optionally substituted C₁₋₄ alkyl. In one aspect of this embodiment, R⁵ represents optionally substituted methyl. In a fifth embodiment, R⁵ represents optionally substituted C₁₋₄ alkoxy. In one aspect according to this embodiment, R⁵ represents optionally substituted methoxy.

Suitably, R⁵ represents hydrogen, halogen, cyano, C₁₋₄ alkyl or C₁₋₄ alkoxy.

Illustratively, R⁵ represents hydrogen, fluoro, cyano, methyl, or methoxy.

In a first embodiment, R⁶ represents hydrogen. In a second embodiment, R⁶ represents halogen. In one aspect of this embodiment, R⁶ represents fluoro. In another aspect of this embodiment, R⁶ represents chloro. In a third embodiment, R⁶ represents cyano. In a fourth embodiment, R⁶ represents optionally substituted C₁₋₄ alkyl. In one aspect of this embodiment, R⁶ represents optionally substituted methyl. In a fifth embodiment, R⁶ represents C₁₋₄ alkoxy. In one aspect according to this embodiment, R⁶ represents methoxy. In a sixth embodiment, R⁶ represents optionally substituted C₁₋₄ alkylamino. In one aspect of this embodiment, R⁶ represents methylamino. In another aspect of this embodiment, R⁶ represents methoxyethyl(methyl)amino. In a further aspect of this embodiment, R⁶ represents (dimethylamino)ethyl-methyl-amino. In a seventh embodiment, R⁶ represents optionally substituted C₃₋₇ heterocycloalkyl. In one aspect of this embodiment, R⁶ represents optionally substituted azetidinyl. In another aspect of this embodiment, R⁶ represents optionally substituted piperazinyl. In a further aspect of this embodiment, R⁶ represents optionally substituted morpholino. In yet a further aspect of this embodiment, R⁶ represents optionally substituted pyrrolydinyl. In a further still aspect of this embodiment, R⁶ represents optionally substituted azaspirohexanyl. In yet a further still aspect of this embodiment, R⁶ represents optionally substituted azaspiroheptanyl. In another aspect of this embodiment, R⁶ represents optionally substituted azabicyclohexanyl. In yet another aspect of this embodiment, R⁶ represents optionally substituted azabicycloheptanyl. In an alternative aspect of this embodiment, R⁶ represents optionally substituted oxa-azaspiroheptanyl. In a further alternative aspect of this embodiment, R⁶ represents optionally substituted oxa-azaspirooctanyl. In an eighth embodiment, R⁶ represents optionally substituted —O—(C₃₋₇ heterocycloalkyl). In one aspect of this embodiment, R⁶ represents optionally substituted (azetidinyl)oxy.

Typical examples of substituents on R⁶ include halogen, hydroxyl, oxo, C₁₋₄ alkylcarboxyhydroxy, and C₁₋₄ alkyl.

Particular examples of substitutents on R⁶ include fluoro, chloro, hydroxyl, oxo, (methyl)carboxy and methyl.

Typically, R⁶ represents hydrogen, chloro, fluoro, cyano, C₁₋₄ alkoxy, C₁₋₄ alkylamino, C₁₋₄ alkyl, C₁₋₄ alkoxy, C₁₋₄ alkyl substituted by one, two or three C₁₋₄ alkylcarboxy or hydroxy, C₃₋₇ heterocycloalkyl substituted by one, two or three substituents selected from oxo, halogen and C₁₋₄ alkyl, or —O—(C₃₋₇ heterocycloalkyl). Typically, when C₁₋₄ alkyl or C₃₋₇ heterocycloalkyl are substituted, the groups are substituted by one or two substituents, most typically one substituent.

Suitably, R⁶ represents hydrogen, chloro, fluoro, cyano, methoxy, methylamino, methyl, (hydroxy)methyl, (methylcarboxy)methyl, (oxo)(methyl)piperazinyl, (difluoro)azetidinyl, azaspirohexanyl, azabicycloheptanyl, (difluoro)azaspirohexanyl, morpholino, (hydroxymethyl)azetidinyl, (fluoroazetidinyl), (methyl)(hydroxyl)azetidinyl, (fluoro)pyrrolidinyl, (difluoro)pyrrolidinyl, (hydroxyl)pyrrolidinyl, (difluoro)azaspiroheptanyl, oxa-azaspirooctanyl, oxa-azaspiroheptanyl, (fluoro)(methyl)azetidinyl, (difluoro)azabicyclohexanyl, (difluoro)azabicycloheptanyl, (methyl)azetidinyl or (azetidinyl)oxy.

In a first embodiment, R⁷ represents hydrogen. In a second embodiment, R⁷ represents optionally substituted C₁₋₄ alkyl. In one aspect of this embodiment, R⁷ represents optionally substituted methyl. In another aspect of this embodiment, R⁷ represents optionally substituted ethyl.

In a third embodiment, R⁷ represents optionally substituted C₃₋₇ cycloalkyl. In one aspect of this embodiment, R⁷ represents optionally substituted cyclopropyl.

Suitably, R⁷ represents hydrogen, C₁₋₄ alkyl, or C₃₋₇ cycloalkyl.

Illustratively, R⁷ represents hydrogen, methyl, ethyl, or cyclopropyl.

In one embodiment, R⁸ represents hydrogen or halogen; or C₁₋₄ alkyl or C₁₋₄ alkoxy, either of which groups may be optionally substituted by one or more substituents. In another embodiment, R⁹ represents hydrogen or halogen; or C₁₋₄ alkyl or C₃₋₇ cycloalkyl, either of which groups may be optionally substituted by one or more substituents. In a further embodiment, R¹⁰ represents hydrogen or halogen; or C₁₋₄ alkyl, which group may be optionally substituted by one or more substituents.

In a particular embodiment, R⁸ represents hydrogen or halogen; or C₁₋₄ alkyl or C₁₋₄ alkoxy, either of which groups may be optionally substituted by one or more substituents; R⁹ represents hydrogen or halogen; or C₁₋₄ alkyl or C₃₋₇ cycloalkyl, either of which groups may be optionally substituted by one or more substituents; and R¹⁰ represents hydrogen or halogen; or C₁₋₄ alkyl, which group may be optionally substituted by one or more substituents.

In a first embodiment, R⁸ represents hydrogen. In a second embodiment, R⁸ represents halogen. In a third embodiment, R⁸ represents optionally substituted C₁₋₄ alkyl. In one aspect of this embodiment, R⁸ represents optionally substituted methyl. In a fourth embodiment, R⁸ represents optionally substituted C₁₋₄ alkoxy. In one aspect of this embodiment, R⁸ represents optionally substituted methoxy.

Suitably, R⁸ represents hydrogen, C₁₋₄ alkyl or C₁₋₄ alkoxy.

Illustratively, R⁸ represents hydrogen, methyl or methoxy.

In a first embodiment, R⁹ represents hydrogen. In a second embodiment, R⁹ represents halogen.

In one aspect according to this embodiment, R⁹ represents fluoro. In a third embodiment, R⁹ represents optionally substituted C₁₋₄ alkyl. In one aspect of this embodiment, R⁹ represents optionally substituted methyl. In a fourth embodiment, R⁹ represents optionally substituted C₃₋₇ cycloalkyl. In one aspect of this embodiment, R⁹ represents optionally substituted cyclopropyl.

Suitably, R⁹ represents hydrogen, halogen, C₁₋₄ alkyl or C₃₋₇ cycloalkyl.

Illustratively, R⁹ represents hydrogen, fluoro, methyl or cyclopropyl.

In a first embodiment, R¹⁰ represents hydrogen. In a second embodiment, R¹⁰ represents halogen. In one aspect according to this embodiment, R¹⁰ represents fluoro. In a third embodiment, R¹⁰ represents optionally substituted C₁₋₄ alkyl.

Suitably, R¹⁰ represents hydrogen or halogen.

Illustratively, R¹⁰ represents hydrogen or fluoro.

Generally, R¹¹ represents —NR^{c}—(CO)—R^{b}.

Suitably, R^{b} represents C₁₋₄ alkyl. Illustratively, R^{b} represents methyl.

Suitably, R^{c} represents C₁₋₄ alkyl. Illustratively, R^{c} represents methyl.

Illustratively, R¹¹ represents (methylcarbonyl)(methyl)amino.

In a first embodiment, B represents B¹. In a second embodiment, B represents B². In a third embodiment, B represents B³. In a fourth embodiment, B represents B⁴. In a fifth embodiment, B represents B⁵. In a sixth embodiment, B represents B⁶. In a seventh embodiment, B represents B⁷.

In an eighth embodiment, B represents B⁸.

In one embodiment, W represents N. In another embodiment, W represents C—H.

In one embodiment, U¹ represents N. In another embodiment, U¹ represents C—H.

In one embodiment, U² represents N. In another embodiment, U² represents C—H.

In one embodiment, Z⁴ represents N. In another embodiment, Z⁴ represents C—R¹³.

In one embodiment, Z⁵ represents N. In another embodiment, Z⁵ represents C—R¹⁴.

In one embodiment, Z⁶ represents N. In another embodiment, Z⁶ represents C—R¹⁵.

In one embodiment, Z⁷ represents N. In another embodiment, Z⁷ represents C—R¹⁶.

In one embodiment, none, one or two of Z⁴, Z⁵, Z⁶ and Z⁷ represents N. Typically, none or one of Z⁴, Z⁵, Z⁶ and Z⁷ represents N.

In a particular embodiment, Z⁴ represents N, Z⁵ represents C—R¹, Z⁶ represents C—R¹⁵ and Z⁷ represents C—R¹⁶.

In another particular embodiment, Z⁴ represents C—R¹³, Z⁵ represents N, Z⁶ represents C—R¹⁵ and Z⁷ represents C—R¹⁶.

In a further particular embodiment, Z⁴ represents C—R¹³, Z⁵ represents C—R¹⁴, Z⁶ represents N and Z⁷ represents C—R¹⁶.

In yet a further particular embodiment, Z⁴ represents C—R¹³, Z⁵ represents C—R¹⁴, Z⁶ represents C—R¹⁵ and Z⁷ represents N.

In yet another further particular embodiment, Z⁴ represents C—R¹, Z⁵ represents C—R¹⁴, Z⁶ represents C—R¹⁵ and Z⁷ represents C—R¹⁶.

In an alternative particular embodiment, Z⁴ represents C—R¹³, Z⁵ represents N, Z⁶ represents C—R¹⁵ and Z⁷ represents N.

In further alternative particular embodiment, Z⁴ represents C—R¹³, Z⁵ represents C—R¹⁴, Z⁶ represents N and Z⁷ represents N.

In a first embodiment, R¹³ represents hydrogen. In a second embodiment, R¹³ represents halogen. In one aspect according to this embodiment R¹³ represents fluoro. In another aspect according to this embodiment, R¹³ represents chloro.

Illustratively, R¹³ represents hydrogen, fluoro or chloro.

In a first embodiment, R¹⁴ represents hydrogen. In a second embodiment, R¹⁴ represents halogen. In one aspect according to this embodiment R¹⁴ represents fluoro. In another aspect according to this embodiment, R¹⁴ represents chloro. In a third embodiment, R¹⁴ represents C₁₋₄ alkoxy. In one aspect according to this embodiment, R¹⁴ represents methoxy.

Illustratively, R¹⁴ represents hydrogen, fluoro, chloro or methoxy.

In a first embodiment, R¹⁵ represents hydrogen. In a second embodiment, R¹⁵ represents halogen. In one aspect according to this embodiment, R¹⁵ represents fluoro. In another aspect according to this embodiment, R¹⁵ represents chloro. In a third embodiment, R¹⁵ represents C₁₋₄ alkyl. In one aspect according to this embodiment, R¹⁵ represents methyl.

Illustratively, R¹⁵ represents hydrogen, fluoro, chloro or methyl.

In a first embodiment, R¹⁶ represents hydrogen. In a second embodiment, R¹⁶ represents halogen. In one aspect according to this embodiment, R¹⁶ represents fluoro. In another aspect according to this embodiment, R¹⁶ represents chloro. In a third embodiment, R¹⁶ represents cyano.

In a fourth embodiment, R¹⁶ represents optionally substituted C₁₋₄ alkyl. In one aspect according to this embodiment, R¹⁶ represents optionally substituted methyl.

Typical examples of optional substituents on R¹⁶ include hydroxy.

Illustratively, R¹⁶ represents hydrogen, fluoro, chloro, cyano or (hydroxy)methyl.

In one embodiment, T represents N. In another embodiment, T represents C—R¹⁷.

In a first embodiment, R¹⁷ represents hydrogen. In a second embodiment, R¹⁷ represents C₁₋₄ alkyl. In one aspect of this embodiment, R¹⁷ represents methyl. In a third embodiment, R¹⁷ represents halogen. In one aspect of this embodiment, R¹⁷ represents fluoro.

Illustratively, R¹⁷ represents hydrogen, methyl or fluoro.

In a first embodiment, R¹⁷′ represents hydrogen. In a second embodiment, R¹⁷′ represents C₁₋₄ alkyl. In one aspect of this embodiment, R¹⁷′ represents methyl. Illustratively, R¹⁷′ represents hydrogen or methyl.

In a first embodiment, Q represents Q¹. In a second embodiment, Q represents Q².

In one embodiment, Z⁸ represents N. In another embodiment, Z⁸ represents C—R³.

In one embodiment, Z¹⁰ represents N. In another embodiment, Z¹⁰ represents C—R¹⁹′.

In one embodiment, one or none of Z⁸ and Z¹⁰ represents N.

In a particular embodiment, Z⁸ represents C—R³ and Z¹⁰ represents C—R¹⁹.

In another particular embodiment, Z⁸ represents N and Z¹⁰ represents C—R¹⁹.

In a further particular embodiment, Z⁸ represents C—R³ and Z¹⁰ represents N.

In one embodiment, Z¹² represents S. In another embodiment, Z¹² represents CR²¹R²². In a further embodiment, Z¹² represents O. In yet another embodiment, Z¹² represents N—H.

In one embodiment, Z¹ represents N. In another embodiment, Z¹³ represents C—R²³.

In one embodiment, Z¹⁴ represents N. In another embodiment, Z¹⁴′ represents C—R²⁴.

In a further embodiment, Z¹² represents S, O or NH; Z¹³ represents C—R²³; and Z¹⁴ represents C—R²⁴.

In yet a further embodiment, Z¹² represents CR²¹R²²; Z¹³ represents N; and Z¹⁴ represents C—R²⁴.

In still a further embodiment, Z¹² represents CR²¹R²²; Z¹³ represents C—R²³; and Z¹⁴ represents N.

In a particular embodiment, Z¹² represents S, Z¹³ represents C—R²³ and Z¹⁴ represents C—R²⁴.

In a more particular embodiment, Q represents an optionally substituted ring selected from the groups represented by Q³, Q⁴, Q⁵ and Q⁶:

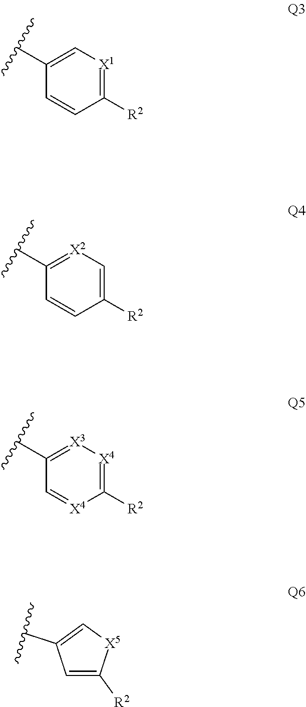

### Wherein

X¹ represents N or C—R³;
X² represents N or C—R¹⁹;
X³ represents C—R¹⁹;
one of the two X⁴ represents C—R²⁰ and the other X⁴ represents C—H;
X⁵ represents S;
Wherein R³, R¹⁹ and R²⁰ are as defined here above.

In a first embodiment, R² represents halogen. In a first aspect of this embodiment R² represents chloro. In a second aspect of this embodiment, R² represents bromo. In a third aspect of this embodiment, R² represents iodo. In a fourth aspect of this embodiment, R² represents fluoro. In a second embodiment, R² represents cyano. In a third embodiment, R² represents optionally substituted C₁₋₄ alkyl. In one aspect of this embodiment, R² represents optionally substituted methyl.

In another aspect of this embodiment, R² represents optionally substituted ethyl. In a further embodiment, R² represents optionally substituted propyl. In a fourth embodiment, R² represents optionally substituted C₃₋₇ cycloalkyl. In one aspect of this embodiment, R² represents optionally substituted cyclopropyl. In a fifth embodiment, R² represents optionally substituted C₁₋₄ alkoxy. In one aspect of this embodiment, R² represents optionally substituted methoxy.

Typical examples of substituents on R² include one, two or three halogen.

Particular examples of substituents on R² include one, two or three fluoro.

Suitably, R² represents halogen, cyanoC₁₋₄ alkyl optionally substituted by one or more halogen, C₃₋₇ cycloalkyl optionally substituted by one or more halogen, or C₁₋₄ alkoxy optionally substituted by one or more halogen.

Particularly, R² represents halogen, cyano, C₁₋₄ alkyl optionally substituted by one or more halogen, C₃₋₇ cycloalkyl optionally substituted by one or more halogen, or C₁₋₄ alkoxy optionally substituted by one or more halogen.

Illustratively, R² represents chloro, bromo, iodo, fluoro, cyano, methyl, difluoromethyl, trifluoromethyl, difluoroethyl, cyclopropyl, difluorocyclopropyl, methoxy, or difluoromethoxy.

In a first embodiment, R³ represents hydrogen. In a second embodiment, R³ represents halogen. In one aspect of this embodiment, R³ represents chloro. In another aspect of this embodiment, R³ represents fluoro. In a third embodiment, R³ represents cyano. In a fourth embodiment, R³ represents optionally substituted C₁₋₄ alkyl. In one aspect of this embodiment, R³ represents optionally substituted methyl.

Suitably, R³ represents hydrogen, halogen, cyano or C₁₋₄ alkyl.

Particularly, R³ represents hydrogen, halogen or C₁₋₄ alkyl.

Illustratively, R³ represents hydrogen, chloro, fluoro or methyl.

In a particular embodiment, R² and R³ together with the group to which they are attached form an optionally substituted cycloalkyl, heterocyclyl, aryl or heteroaryl. In one aspect according to this embodiment R² and R³ together with the group to which they are attached form an optionally substituted benzotriazolyl. In another aspect according to this embodiment, R² and R³ together with the group to which they are attached form an optionally substituted indazolyl. In a further aspect according to this embodiment, R² and R³ together with the group to which they are attached form an optionally substituted indanyl. In yet a further aspect according to this embodiment, R² and R³ together with the group to which they are attached form an optionally substituted benzodioxolyl. In still yet a further aspect according to this embodiment, R² and R³ together with the group to which they are attached form an optionally substituted isoquinolyl.

Typical optional substituents on the cycloalkyl, heterocyclyl, aryl or heteroaryl include halogen and C₁₋₄ alkyl optionally substituted by one, two or three halogen.

Suitable optional substituents on the cycloalkyl, heterocyclyl, aryl or heteroaryl include fluoro, chloro and methyl optionally substituted by one, two or three halogen.

Specific examples of optional substituents on the cycloalkyl, heterocyclyl, aryl or heteroaryl include fluoro, chloro, methyl and difluoromethyl.

Suitably, R² and R³ together with the group to which they are attached form (methyl)indazolyl, (difluoromethyl)indazolyl, (methyl)triazolyl, (difluoro)indanyl, (difluoro)benzodioxolyl, (fluoro)isoquinolyl or (chloro)isoquinolyl.

In a first embodiment, R¹⁸ represents hydrogen. In a second embodiment, R¹⁸ represents halogen. In one aspect of this embodiment, R¹⁸ represents fluoro. In a third embodiment, R¹⁸ represents C₁₋₄ alkyl. In one aspect of this embodiment, R¹⁸ represents methyl.

Suitably, R¹⁸ represents hydrogen or fluoro.

In a first embodiment, R¹⁹ represents hydrogen. In a second embodiment, R¹⁹ represents halogen. In one aspect of this embodiment, R¹⁹ represents fluoro. In a third embodiment, R¹⁹ represents C₁₋₄ alkyl. In one aspect of this embodiment, R¹⁹ represents methyl.

Suitably, R¹⁹ represents hydrogen or fluoro.

In a first embodiment, R²⁰ represents hydrogen. In a second embodiment, R²⁰ represents halogen. In one aspect of this embodiment, R²⁰ represents chloro. In another aspect of this embodiment, R²⁰ represents fluoro. In a third embodiment, R²⁰ represents optionally substituted C₁₋₄ alkyl. In one aspect of this embodiment, R²⁰ represents optionally substituted methyl.

Suitably, R²⁰ represents hydrogen, halogen, or C₁₋₄ alkyl.

Particularly, R²⁰ represents hydrogen or halogen.

Illustratively, R²⁰ represents hydrogen or fluoro.

In a first embodiment, R²¹ represents hydrogen. In a second embodiment, R²¹ represents halogen. In one aspect of this embodiment, R²¹ represents fluoro. In a third embodiment, R²¹ represents C₁₋₄ alkyl. In one aspect of this embodiment, R²¹ represents methyl.

Suitably, R²¹ represents hydrogen or fluoro.

In a first embodiment, R²² represents hydrogen. In a second embodiment, R²² represents halogen. In one aspect of this embodiment, R²² represents fluoro. In a third embodiment, R²² represents C₁₋₄ alkyl. In one aspect of this embodiment, R²² represents methyl.

Suitably, R²² represents hydrogen or fluoro.

In a first embodiment, R²³ represents hydrogen. In a second embodiment, R²³ represents halogen. In one aspect of this embodiment, R²³ represents fluoro. In a third embodiment, R²³ represents C₁₋₄ alkyl. In one aspect of this embodiment, R²³ represents methyl.

Suitably, R²³ represents hydrogen or fluoro.

In a first embodiment, R²⁴ represents hydrogen. In a second embodiment, R²⁴ represents halogen. In one aspect of this embodiment, R²⁴ represents fluoro. In a third embodiment, R²⁴ represents C₁₋₄ alkyl. In one aspect of this embodiment, R²⁴ represents methyl.

Suitably, R²⁴ represents hydrogen or fluoro.

In a first particular embodiment, Y represents N—R^{a} or CR^{1a}R^{1b}; R^{a} represents methyl; R^{1a} represents hydrogen, halogen, C₁₋₄ alkyl, C₁₋₄ alkyl substituted by hydroxyl, C₁₋₄ alkyl substituted by C₁₋₄ alkoxy, or C₁₋₄ alkoxy; R^{1b} represents hydrogen or C₁₋₄ alkyl; A, together with the points of attachment to the remainder of the molecule, V³ and V⁴, represents an optionally substituted aryl or heteroaryl selected from the groups represented by A¹, A², A³, A⁴, A⁵, A⁶ and A⁷; Z¹ represents N or C—R⁴, Z² represents N or C—R⁵, and Z³ represents N or C—R⁶, wherein one or none of Z¹, Z² and Z³ represents N; Re represents hydrogen or fluoro; R⁴ represents hydrogen, halogen, cyano, amino, C₁₋₄ alkyl, C₁₋₄ alkoxy, C₁₋₄ alkyl substituted by one, two or three halogen or hydroxyl, or C₁₋₄ alkoxy substituted by one, two or three C₁₋₄ alkoxy; R⁵ represents hydrogen, halogen, cyano, C₁₋₄ alkyl or C₁₋₄ alkoxy; R⁶ represents hydrogen, chloro, cyano, C₁₋₄ alkoxy, C₁₋₄ alkylamino, C₁₋₄ alkyl, C₁₋₄ alkoxy, C₁₋₄ alkyl substituted by one, two or three C₁₋₄ alkylcarboxy or hydroxy, C₃₋₇ heterocycloalkyl substituted by one, two or three substituents selected from oxo, halogen and C₁₋₄ alkyl, or —O—(C₃₋₇ heterocycloalkyl); R⁷ represents hydrogen, C₁₋₄ alkyl, or C₃₋₇ cycloalkyl; R⁸ represents hydrogen, C₁₋₄ alkyl or C₁₋₄ alkoxy; R⁹ represents hydrogen, halogen, C₁₋₄ alkyl or C₃₋₇ cycloalkyl; R¹⁰ represents hydrogen or halogen; R¹¹ represents —NR^{c}—(CO)—R^{b}; R^{b} represents C₁₋₄ alkyl; R^{c} represents C₁₋₄ alkyl; B, together with the points of attachment to the remainder of the molecule, V¹ and V², represents an optionally substituted aryl or heteroaryl selected from the groups represented by B¹, B², B³, B⁴, B⁵, B⁶, B⁷ and B⁸; V¹ represents C for B¹, B², B³, B⁴, B⁵, B⁶ and B⁷ and represents N for B⁸; V² represents C for B¹, B², B⁴, B⁶, B⁷, and B⁸ and represents N for B³ and B⁵; W, U¹ and U² represent independently N or C—H; Z⁴ represents N or C—R¹³, Z⁵ represents N or C—R¹⁴, Z⁶ represents N or C—R¹⁵, and Z⁷ represents N or C—R¹⁶, wherein none or one of Z⁴, Z⁵, Z⁶ and Z⁷ represents N; T represents N or C—R¹⁷; R¹² represents hydrogen; R¹³ represents hydrogen or halogen; R¹⁴ represents hydrogen, halogen or C₁₋₄ alkoxy; R¹⁵ represents hydrogen, halogen or C₁₋₄ alkyl; R¹⁶ represents hydrogen, halogen, cyano or optionally substituted C₁₋₄ alkyl; R¹⁷ represents hydrogen, halogen or C₁₋₄ alkyl; R¹⁷′ represents hydrogen or C₁₋₄ alkyl; Q represents a ring selected from the groups represented by Q¹ and Q²; Z⁸ represents N or C—R³, Z⁹ represents C—R¹⁸, Z¹⁰ represents N or C—R¹⁹, and Z¹¹ represents C—R²⁰, wherein one or none of Z⁸ and Z¹⁰ represents N; Z¹² represents S, Z¹³ represents C—R²³, and Z¹⁴ represents C—R²⁴; R² represents halogen, cyanoC₁₋₄ alkyl optionally substituted by one or more halogen, C₃₋₇ cycloalkyl optionally substituted by one or more halogen, or C₁₋₄ alkoxy optionally substituted by one or more halogen; R³ represents hydrogen, halogen, cyano or C₁₋₄ alkyl; or R² and R³ together with the group to which they are attached form an indazolyl, indazolyl, triazolyl, indanyl, benzodioxolyl, isoquinolyl or isoquinolyl; R¹⁸ represents hydrogen or fluoro; R¹⁹ represents hydrogen or fluoro; R²⁰ represents hydrogen or halogen; R²³ represents hydrogen or fluoro; R²⁴ represents hydrogen or fluoro.

In a more particular embodiment, Y represents N—R^{a} or CR^{1a}R^{1b}; R^{a} represents methyl; R^{1a} represents hydrogen, deuterium, fluoro, hydroxyl, methyl, deuterated methyl (—CD₃), hydroxymethyl, methoxymethyl, or methoxy; R^{1b} represents hydrogen or methyl; A, together with the points of attachment to the remainder of the molecule, V³ and V⁴, represents an optionally substituted aryl or heteroaryl selected from the groups represented by A¹, A², A³, A⁴, A⁵, A⁶ and A⁷; Z¹ represents N or C—R⁴, Z² represents N or C—R^{5′}, and Z³ represents N or C—R⁶, wherein one or none of Z¹, Z² and Z³ represents N; Re represents hydrogen or fluoro; R⁴ represents hydrogen, chloro, fluoro, cyano, hydroxyl, amino, methyl, ethyl, difluoromethyl, hydroxymethyl, cyclopropyl, methoxy, ethoxy, or (methoxy)ethoxy; R⁵ represents hydrogen, fluoro, cyano, methyl, or methoxy; R⁶ represents hydrogen, chloro, fluoro, cyano, methoxy, methylamino, methyl, (hydroxy)methyl, (methylcarboxy)methyl, (oxo)(methyl)piperazinyl, (difluoro)azetidinyl, azaspirohexanyl, azabicycloheptanyl, (difluoro)azaspirohexanyl, morpholino, (hydroxymethyl)azetidinyl, (fluoroazetidinyl), (methyl)(hydroxyl)azetidinyl, (fluoro)pyrrolidinyl, (difluoro)pyrrolidinyl, (hydroxyl)pyrrolidinyl, (difluoro)azaspiroheptanyl, oxa-azaspirooctanyl, oxa-azasprioheptanyl, (fluoro)(methyl)azetidinyl, (difluoro)azabicyclohexanyl, (diflouro)azabicycloheptanyl, (methyl)azetidinyl, or (azetidinyl)oxy; R⁷ represents hydrogen, methyl, ethyl, or cyclopropyl; R⁸ represents hydrogen, methyl or methoxy; R⁹ represents hydrogen, fluoro, methyl or cyclopropyl; R¹⁰ represents hydrogen or fluoro; R¹¹ represents —NR^{c}—(CO)—R^{b}; R^{b} represents methyl; R^{c} represents methyl; B, together with the points of attachment to the remainder of the molecule, V¹ and V², represents an optionally substituted aryl or heteroaryl selected from the groups represented by B¹, B², B³, B⁴, B⁵, B⁶, B⁷ and B⁸; V¹ represents C for B¹, B², B³, B⁴, B⁵, B⁶ and B⁷ and represents N for B⁸; V² represents C for B¹, B², B⁴, B⁶, B⁷, and B⁸ and represents N for B³ and B⁵; W, U¹ and U² represent independently N or C—H; Z⁴ represents N or C—R¹³, Z⁵ represents N or C—R¹⁴, Z⁶ represents N or C—R¹⁵, and Z⁷ represents N or C—R¹⁶, wherein none, one or two of Z⁴, Z⁵, Z⁶ and Z⁷ represents N; T represents N or C—R¹⁷; R¹² represents hydrogen; R¹³ represents hydrogen, fluoro or chloro; R¹⁴ represents hydrogen, fluoro, chloro or methoxy; R¹⁵ represents hydrogen, fluoro, chloro or methyl; R¹⁶ represents hydrogen, fluoro, chloro, cyano or (hydroxy)methyl; R¹⁷ represents hydrogen, methyl or fluoro; R¹⁷′ represents hydrogen or methyl; Q represents an optionally substituted ring selected from the groups represented by Q³, Q⁴, Q⁵ and Q⁶; X¹ represents N or C—R³; X² represents N or C—R¹⁹; X³ represents C—R¹⁹; one of the two X⁴ represents C—R²⁰ and the other X⁴ represents C—H; X⁵ represents S; R² represents chloro, bromo, iodo, fluoro, cyano, methyl, difluoromethyl, trifluoromethyl, difluoroethyl, cyclopropyl, difluorocyclopropyl, methoxy, or difluoromethoxy; R³ represents hydrogen, chloro, fluoro or methyl; or R² and R³ together with the group to which they are attached form (methyl)indazolyl, (difluoromethyl)indazolyl, (methyl)triazolyl, (difluoro)indanyl, (difluoro)benzodioxolyl, (fluoro)isoquinolyl and (chloro)isoquinolyl; R¹⁹ represents hydrogen or fluoro; R²⁰ represents hydrogen or fluoro.

In a first particular embodiment, the present invention relates to a particular subclass of compounds of formula (I) represented by formula (IA),

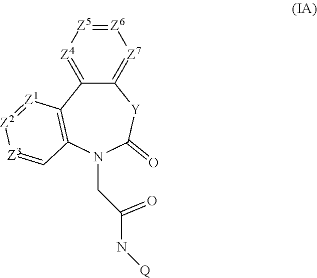

Wherein Q, Y, Z¹, Z², Z³, Z⁴, Z⁵, Z⁶ and Z⁷ are as defined here above.

In a particular aspect of this embodiment, the present invention relates to a particular subclass of compounds of formula (IA) represented by formula (IA-a),

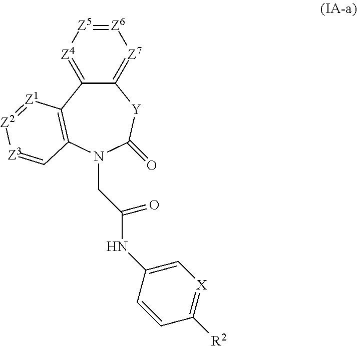

Wherein X, Y, R², Z¹, Z², Z³, Z⁴, Z⁵, Z⁶ and Z⁷ are as defined here above.

In a second particular embodiment, the present invention relates to a particular subclass of compounds of formula (I) represented by formula (IB),

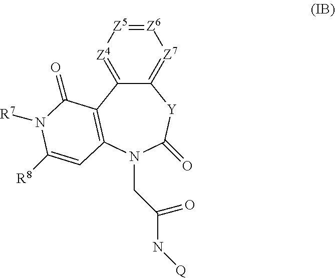

Wherein Q, Y, Z⁴, Z⁵, Z⁶, Z⁷, R⁷ and R⁸ are as defined here above.

In a particular aspect of this embodiment, the present invention relates to a particular subclass of compounds of formula (IB) represented by formula (IB-a),

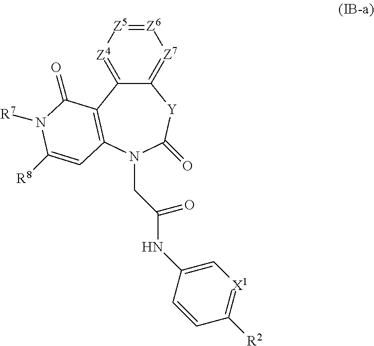

Wherein X¹, Y, Z⁴, Z⁵, Z⁶, Z⁷, R², R⁷ and R⁸ are as defined here above.

In a third particular embodiment, the present invention relates to a particular subclass of compounds of formula (I) represented by formula (IC),

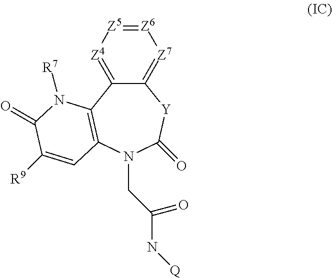

Wherein Q, Y, Z⁴, Z⁵, Z⁶, Z⁷, R⁷ and R⁹ are as defined here above.

In a particular aspect of this embodiment, the present invention relates to a particular subclass of compounds of formula (IC) represented by formula (IC-a),

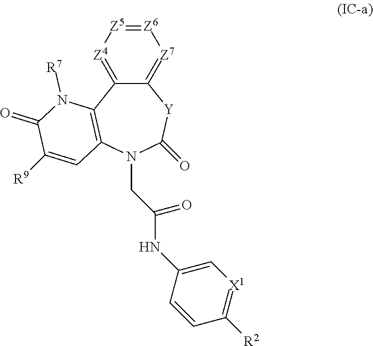

Wherein X, Y, Z⁴, Z⁵, Z⁶, Z⁷, R², R⁷ and R⁹ are as defined here above.

In a fourth particular embodiment, the present invention relates to a particular subclass of compounds of formula (I) represented by formula (ID),

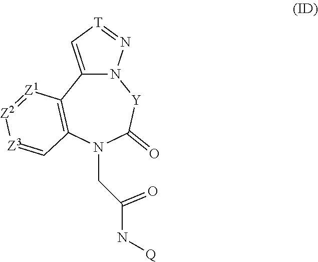

Wherein Q, Y, Z¹, Z², Z³, and T are as defined here above.

In a particular aspect of this embodiment, the present invention relates to a particular subclass of compounds of formula (ID) represented by formula (ID-a),

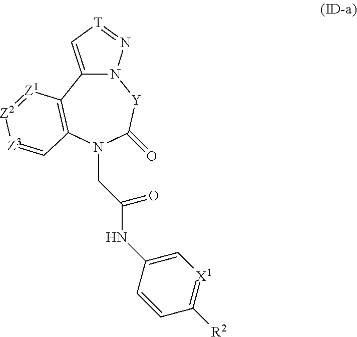

Wherein X, Y, R², Z¹, Z², Z³, and T are as defined here above.

In a fifth particular embodiment, the present invention relates to a particular subclass of compounds of formula (I) represented by formula (IE),

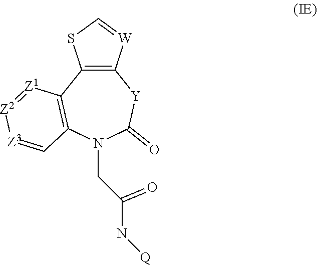

Wherein Q, Y, Z¹, Z², Z³ and W are as defined here above.

In a particular aspect of this embodiment, the present invention relates to a particular subclass of compounds of formula (IE) represented by formula (IE-a),

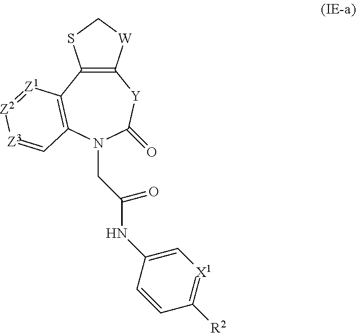

Wherein X, Y, R², Z¹, Z², Z³ and W are as defined here above.

Specific novel compounds in accordance with the present invention include each of the compounds whose preparation is described in the accompanying Examples, their individual stereoisomers, and pharmaceutically acceptable salts and solvates thereof.

Therefore, in a particular aspect, the present invention relates to compounds of formula (I) as described in the accompanying Examples 1-424.

In a most particular embodiment, the present invention relates to a subclass of compounds of formula (I) represented by formula (IF),

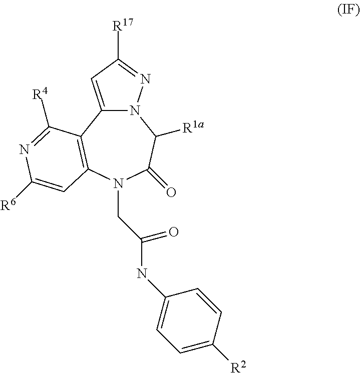

Wherein R^{1a}, R^{2′} R⁴, R⁶ and R¹⁷ are as defined here above. In the compounds of formula (IF), typically:
R^{1a} is methyl or hydroxyl;
R² is difluoromethyl or difluoroethyl;
R⁴ is hydrogen, chloro or fluoro:
R⁶ is methyl;
R¹⁷ is hydrogen or fluoro.

In a further most particular embodiment, the present invention relates to a subclass of compounds of formula (I) represented by formula (IG),

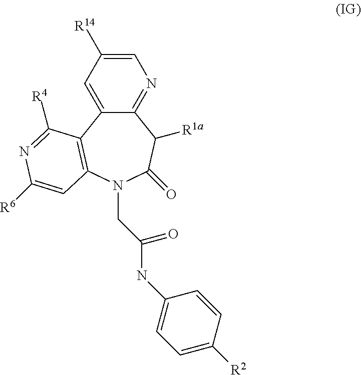

Wherein R^{1a}, R², R⁴ R⁶ and R¹⁴ are as defined here above. In the compounds of formula (IG), typically:
R^{1a} is methyl or hydroxyl;
R² is difluoromethyl or difluoroethyl;
R⁴ is hydrogen, chloro or fluoro;
R⁶ is methyl; and
R¹⁴ is hydrogen or fluoro.

In yet a further most particular embodiment, the present invention relates to a subclass of compounds of formula (I) represented by formula (IH),

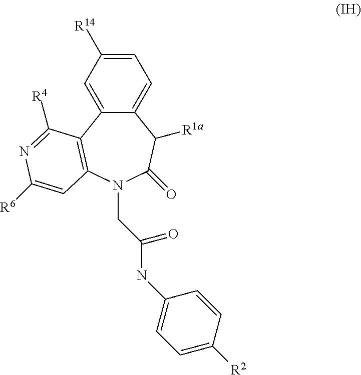

Wherein R^{1a}, R², R⁴, R⁶ and R¹⁴ are as defined here above. In the compounds of formula (IH), typically:
R^{1a} is methyl or hydroxyl;
R² is difluoromethyl or difluoroethyl;
R⁴ is hydrogen, chloro or fluoro;
R⁶ is methyl; and
R¹⁴ is hydrogen or fluoro.

In still a further most particular embodiment, the present invention relates to a subclass of compounds of formula (I) represented by formula (IJ),

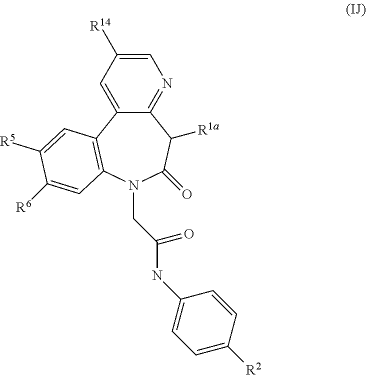

Wherein R^{1a}, R², R⁵ R⁶ and R¹⁴ are as defined here above. In the compounds of formula (IJ), typically:
R^{1a} is methyl or hydroxyl;
R² is difluoromethyl or difluoroethyl;
R⁵ is cyano;
R⁶ is methyl; and
R¹⁴ is hydrogen or fluoro.

In another most particular embodiment, the present invention relates to a subclass of compounds of formula (I) represented by formula (IK),

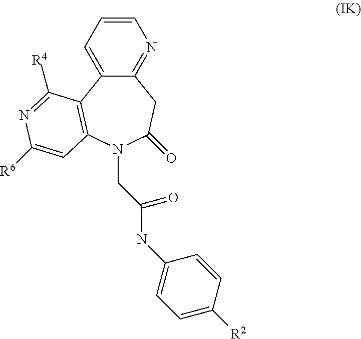

Wherein R², R⁴ and R⁶ are as defined here above. In the compounds of formula (IK′), typically:
R² is difluoromethyl or difluoroethyl;
R⁴ is hydrogen, chloro or fluoro; and
R⁶ is 2,2-difluoro-5-azaspiro[2.3]hexan-5-yl.

In yet another most particular embodiment, the present invention relates to a subclass of compounds of formula (I) represented by formula (IL),

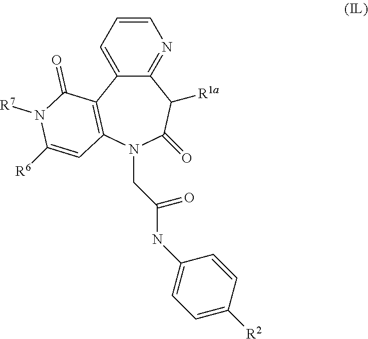

Wherein R^{1a}, R², R⁷ and R⁶ are as defined here above. In the compounds of formula (IL), typically:
R^{1a} is methy or hydroxyl;
R² is difluoromethyl or trifluoromethyl;
R⁷ is hydrogen or methyl; and
R⁶ is methyl.

In still another most particular embodiment, the present invention relates to a subclass of compounds of formula (I) represented by formula (IM),

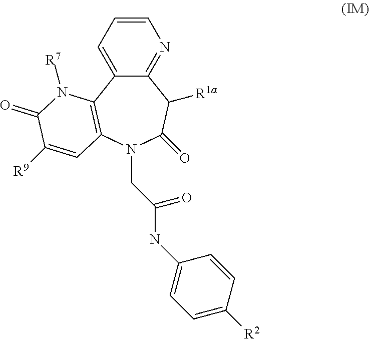

Wherein R^{1a}, R², R⁷ and R⁹ are as defined here above. In the compounds of formula (IM), typically:
R^{1a} is methy or hydroxyl;
R² is difluoromethyl or trifluoromethyl;
R⁷ is hydrogen or methyl; and
R⁹ is methyl.

In one aspect of the most particular embodiments in which the compounds are represented by formula (IF), formula (IG), formula (IH), formula (IJ), formula (IK), formula (IL) or formula (IM), and R² is difluoroethyl, then R² is specifically —CF₂CH₃.

The present invention also provides a compound of formula (I) as defined above or a pharmaceutically acceptable salt thereof, for use in therapy.

In particular, the present invention provides a compound of formula (I) as defined above, or a pharmaceutically acceptable salt thereof, for use in the treatment of diseases and/or disorders in which System Xc⁻ plays a role.

In the following aspects, the compound of formula (I) as defined above may be an inhibitor of the System Xc⁻ antiporter.

In a first aspect, the present invention provides a compound of formula (I) as defined above, or a pharmaceutically acceptable salt thereof, for use in the treatment of cancers where System Xc-plays a role, in epilepsy syndromes where System Xc⁻ plays a role, or in cancer treatment resistance

In a first embodiment according to this aspect, the present invention provides a compound of formula (I) as defined above, or a pharmaceutically acceptable salt thereof, for use in the treatment of cancers where System Xc⁻ plays a role.

In particular, the present invention provides a compound of formula (I) as defined above, or a pharmaceutically acceptable salt thereof for use in the treatment of glioma, colon carcinoma, colorectal carcinoma, lung cancer, esophageal carcinoma, triple negative breast cancer, hepatocellular carcinoma, adrenocortical carcinoma, kidney carcinoma, mesothelioma, sarcoma, uveal melanoma, gastric cancer, pancreatic carcinoma or leukemia.

In a second embodiment according to this aspect, the present invention provides a compound of formula (I) as defined above for use in the treatment of epilepsy syndromes where System Xc-plays a role.

In particular, the present invention provides a compound of formula (I) as defined above, or a pharmaceutically acceptable salt thereof, for use in the treatment of epileptogenesis, glutamate induced seizures, glioma associated epilepsy, focal cortical dysplasia or tuberous sclerosis.

In a third embodiment, the present invention provides compounds of formula (I′) or formula (I) for use in the treatment of cancer treatment resistance.

In particular, the present invention provides a compound of formula (I) as defined above, or a pharmaceutically acceptable salt thereof, for use in the treatment of multidrug resistance in several cancer types.

In a second aspect, the present invention provides for the use of a compound of or formula (I) as defined above, or a pharmaceutically acceptable salt thereof, for the manufacture of a medicament useful for the treatment of diseases and/or disorders in which system Xc-cystine/glutamate antiporter plays a role.

In a first embodiment of this aspect, the present invention provides for the use of a compound of formula (I) as defined above, or a pharmaceutically acceptable salt thereof, for the manufacture of a medicament useful for the treatment of cancers where System Xc⁻ plays a role.

In particular, the present invention provides for the use of a compound of formula (I) as defined above, or a pharmaceutically acceptable salt thereof for the manufacture of a medicament useful for the treatment glioma, colon carcinoma, colorectal carcinoma, lung cancer, esophageal carcinoma, triple negative breast cancer, hepatocellular carcinoma, adrenocortical carcinoma, kidney carcinoma, mesothelioma, sarcoma, uveal melanoma, gastric cancer, pancreatic carcinoma or leukemia.

In a second embodiment according to this aspect, the present invention provides for the use of a compound of formula (I) as defined above, or a pharmaceutically acceptable salt thereof, for the manufacture of a medicament useful for the treatment of epilepsy syndromes where System Xc-plays a role.

In particular, the present invention provides a compound of formula (I) as defined above, or a pharmaceutically acceptable salt thereof, for the manufacture of a medicament useful for the treatment of epileptogenesis, glutamate induced seizures, glioma associated epilepsy, focal cortical dysplasia or tuberous sclerosis.

In a third embodiment according to this aspect, the present invention provides for the use of a compound of formula (I) as defined above, or a pharmaceutically acceptable salt thereof, for the manufacture of a medicament useful for cancer treatment resistance.

In particular, the present invention provides for the use of a compound of formula (I) as defined above, or a pharmaceutically acceptable salt thereof, for the manufacture of a medicament useful for the treatment of multidrug resistance in several cancer types. In a third aspect, the present invention provides a method for the treatment of disorders for which the administration of inhibitors of the System Xc⁻ is indicated, which comprises administering to a patient in need of such treatment an effective amount of a compound of formula (I) as defined above, or a pharmaceutically acceptable salt thereof.

In a first embodiment according to this aspect, the present invention provides a method for the treatment of cancers where System Xc⁻ plays a role, which comprises administering to a patient in need of such treatment an effective amount of a compound of formula (I) as defined above, or a pharmaceutically acceptable salt thereof.

In particular, the present invention provides a method for the treatment of glioma, colon carcinoma, colorectal carcinoma, lung cancer, esophageal carcinoma, triple negative breast cancer, hepatocellular carcinoma, adrenocortical carcinoma, kidney carcinoma, mesothelioma, sarcoma, uveal melanoma, gastric cancer, pancreatic carcinoma or leukemia, which comprises administering to a patient in need of such treatment of an effective amount of a compound of formula (I) as defined above, or a pharmaceutically acceptable salt thereof.

In a second embodiment according to this aspect, the present invention provides a method for the treatment of epilepsy syndromes where System Xc⁻ plays a role, which comprises administering to a patient in need of such treatment an effective amount of a compound of formula (I) as defined above, or a pharmaceutically acceptable salt thereof.

In particular, the present invention provides a method for the treatment of epileptogenesis, glutamate induced seizures, glioma associated epilepsy, focal cortical dysplasia or tuberous sclerosis, which comprises administering to a patient in need of such treatment an effective amount of a compound of formula (I) as defined above, or a pharmaceutically acceptable salt thereof.

In a third embodiment according to this aspect, the present invention provides a method for the treatment of cancer treatment resistance, which comprises administering to a patient in need of such treatment an effective amount of a compound of formula (I) as defined above, or a pharmaceutically acceptable salt thereof. In particular, the present invention provides a method for the treatment of multidrug resistance in several cancer types, which comprises administering to a patient in need of such treatment an effective amount of a compound of or formula (I) as defined above, or a pharmaceutically acceptable salt thereof. As used herein, the term “patient” refers to a mammal that is afflicted with one or more disorders associated with function or expression of System Xc⁻. It will be understood that the most preferred patient is a human.

It is also recognized that one skilled in the art may affect the disorders by treating a patient presently afflicted with the disorders, or by prophylactically treating a patient afflicted with the disorders with an effective amount of the compound of Formula (I). Thus, the terms “treatment” and “treating” are intended to refer to all processes wherein there may be a slowing, interrupting, arresting, controlling, or stopping of the progression of the disorders described herein, and is intended to include prophylactic treatment of such disorders, but does not necessarily indicate a total elimination of all disorder symptoms.

Activity in any of the above-mentioned therapeutic indications or disorders can of course be determined by carrying out suitable clinical trials in a manner known to a person skilled in the relevant art for the particular indication and/or in the design of clinical trials in general.

For use in medicine, the salts of the compounds of formula (I) will be pharmaceutically acceptable salts. Other salts may, however, be useful in the preparation of the compounds of use in the invention or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compound of formula (I) include acid addition salts which may, for example, be formed by mixing a solution of the compound of or formula (I) with a solution of a pharmaceutically acceptable acid.

The present invention includes within its scope solvates of the compounds of formula (I) above. Such solvates may be formed with common organic solvents or water.

The present invention also includes within its scope co-crystals of the compounds of formula (I) above. The technical term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity (see Pharmaceutical Salts and Co-crystals, ed. J. Wouters & L. Quere, RSC Publishing, 2012).

Compounds according to the present invention may exist in different polymorphic forms. Although not explicitly indicated in the above formula, such forms are intended to be included within the scope of the present invention.

The invention also includes within its scope pro-drug forms of the compounds of formula (I) and its various sub-scopes and sub-groups.

For treating diseases, compounds of formula (I) or their pharmaceutically acceptable salts may be employed at an effective daily dosage and administered in the form of a pharmaceutical composition.

Therefore, another embodiment of the present invention concerns a pharmaceutical composition comprising an effective amount of a compound of formula (I) or a pharmaceutically acceptable salt thereof in combination with a pharmaceutically acceptable diluent or carrier.

To prepare a pharmaceutical composition according to the invention, one or more of the compounds of formula (I) or a pharmaceutically acceptable salt thereof is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, parenteral, intranasal, or intratumoral.

Pharmaceutical compositions comprising compounds according to the invention can, for example, be administered orally, parenterally, i.e. intravenously, intramuscularly or subcutaneously, intrathecally, by inhalation, intranasally or by ophthalmic administration.

Pharmaceutical compositions suitable for oral administration can be solids or liquids and can, for example, be in the form of tablets, pills, dragees, gelatin capsules, solutions, syrups, chewing-gums and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycolumn or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus, the quantity of compound of formula (I) in compositions for oral administration is at least 0.5% by weight and can be up to 80% by weight with respect to the total weight of the composition.

In accordance with the invention, it has also been found that the compounds of formula (I′) or formula (I) or the pharmaceutically acceptable salts thereof can be administered alone or in combination with other pharmaceutically active ingredients.

In particular, compounds of formula (I) according to the present invention could be combined with other active ingredients that increase intracellular reactive oxygen species, regulate amino acid metabolism or with immunotherapeutic agents.

In compositions for parenteral administration, the quantity of compound of formula (I) present is at least 0.5% by weight and can be up to 33% by weight with respect to the total weight of the composition. For the preferred parenteral compositions, the dosage unit is in the range 0.5 mg to 3000 mg of compounds of formula (I).

The daily dose can fall within a wide range of dosage units of compound of formula (I) and is generally in the range 0.5 to 3000 mg. However, it should be understood that the specific doses can be adapted to particular cases depending on the individual requirements, at the physician's discretion.

### Synthetic Schemes

It will be apparent to the person skilled in the art that there are various synthetic pathways that can lead to the compounds according to the invention. The following processes are aimed at illustrating some of these synthetic pathways but should not be construed in any way as a limitation on how the compounds according to the invention should be made.

During any of the below synthetic sequences, it may be necessary and/or desirable to protect sensitive or reactive groups on any of the molecules concerned. This may be achieved by means of conventional protecting groups (PG), such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} edition, 1999. The protecting groups may be removed at any convenient subsequent stage utilising methods known from the art.

The compounds of Formula (I) according to the invention can be prepared analogously to conventional methods as understood by the person skilled in the art of synthetic organic chemistry.

In the following description of general synthetic methods, “DCM” means dichloromethane; “DIPEA” refers to N,N-di-iso-propylethylamine; “DMF” refers to N,N-dimethylformamide; “DMSO” refers to dimethylsulfoxide; “EDC” refers to 1-Ethyl-3-carbodiimide hydrochloride; “; “TEA” refers to triethylamine; “THF” refers to tetrahydrofuran; “HATU” refers to hexafluorophosphate azabenzotriazole tetramethyl uranium; “HBTU” refers to hexafluorophosphate benzotriazole tetramethyl uronium; “HOBt” refers to hydroxybenzotriazole”; “TCFH” refers to chloro-N,N,N′,N′-tetramethylformamidinium hexafluorophosphate; and “NMI” refers to N-methylimidazole.

Compounds of formula (I) according to the invention can be prepared by analogous methods, as understood by the person skilled in the art of synthetic organic chemistry. The following description of synthetic schemes provides for means of preparing compounds of formula (I). However, analogous methods may be used in the preparation of compounds of formula (I′). According to one embodiment, compounds having the general Formula (I) wherein Q represents Q³, may be prepared by reaction of a compound of formula (2) with an amide of formula (4) or by reaction of a compounds of formula (3) with an aromatic amine of formula (5) according to the equation:

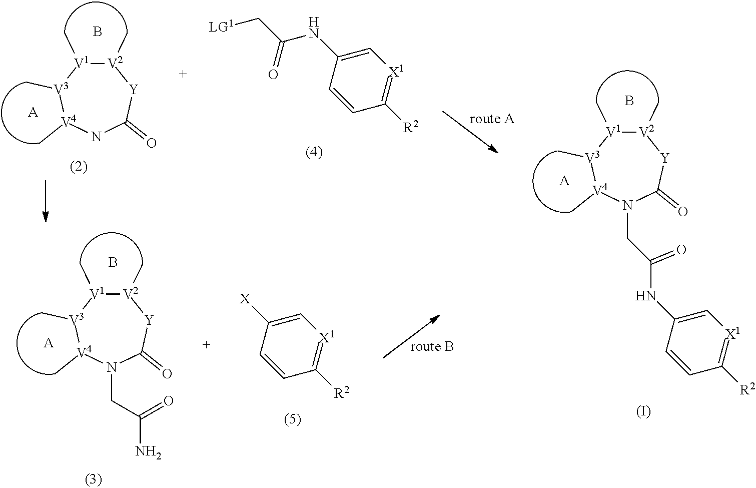

wherein A, B, V¹, V², V³, V⁴, Y, X¹ and R² are as defined above for compound of Formula (I) and where LG¹ is a halogen atom or a leaving group such as a mesylate or tosylate; and LG² is hydroxy, alkoxy, or halogen.

The reaction following route A may be performed with a base such as trialkyl amines, inorganic carbonates or pyridines, with or without the presence of an iodide salt such as KI or NaI in a suitable solvent such as DMSO, DMF, sulfolane, acetonitrile, or THF.

Alternatively, compounds of Formula (I) may be prepared following route B by reaction of a carboxylic acid or carboxylic derivatives of formula (3) with aromatic amines (5) following procedures for the formation of an amide from carboxylic acids or carboxylic derivatives and amines known to the person skilled in the art. The reaction following route B, when LG² is a halogen such as chlorine, may be performed with a base such as trialkyl amines, inorganic carbonates, or pyridines in a suitable solvent such as DCM, DMSO, DMF, sulfolane, acetonitrile, or THF. When LG² is hydroxy, the reaction may be performed with similar bases and in the presence of an amide coupling reagent such as HBTU, HATU, TCFH/NMI, EDC/HOBt, or according to any other method known to the person skilled in the art. Alternatively, compounds of formula (3) in which LG² is hydroxy can be transformed into compounds of formula (3) in which LG² is chloro by reaction with sulfonyl chloride or thionyl chloride in the presence or absence of catalytic DMF, in a suitable solvent such as DCM or THF at room or at higher temperatures such as 70° C.

Compounds of formula (3) where LG² is an alkoxy such as OMe, OEt, or OtBu may be prepared by reaction of intermediate (2) with an alpha-chloro ester or an alpha-bromo ester such as methyl 2-bromoacetate, ethyl 2-bromoacetate, or tert-butyl 2-bromoacetate in the presence of a base such as potassium carbonate in a polar solvent such as DMF at room temperature or by any method known to the person skilled in the art. Further basic or acid ester hydrolysis known to the person skilled in the art may be used to form compounds of formula (3) in which LG² is OH.

Alternatively, compounds of Formula (I), wherein Q represents Q³, may be prepared by reaction of an intermediate of Formula (3) wherein LG² is NH₂, hereinafter referred to as (3′), with a compound of formula (5′) wherein X is sulfonate such as a triflate, an halogen such as chloro or bromo in the presence of a catalytic amount of a palladium catalyst. This reaction, the “Buchwald amide coupling”, is known to the person skilled in the art.

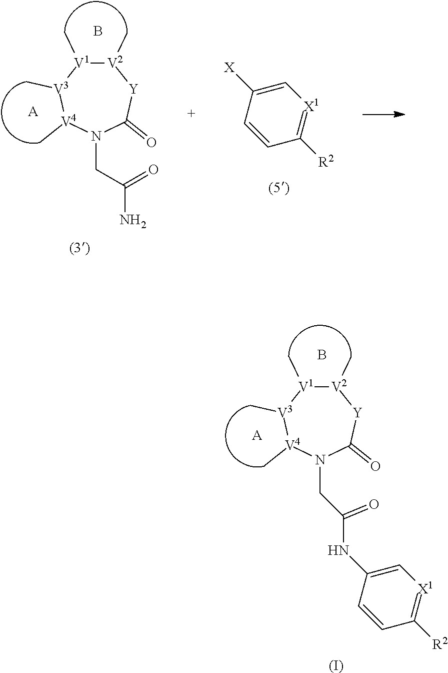

Compounds of Formula (5′) are either commercially available or maybe prepared by any G-7C method known to the person skilled in the art.

Compounds of Formula (3′) may be prepared by reaction of a compound of Formula (2) with an alpha-halogeno amide such as iodoacetamide in the presence of a base such as potassium carbonate in a polar solvent such as DMF at room temperature or by any other method known to the person skilled in the art. Alternatively, compounds of Formula (3′) may be prepared by reaction of a carboxylic acid or carboxylic acid derivatives of formula (3) with ammonia following procedures for the formation of an amide from carboxylic acids or carboxylic derivatives and amines known to the person skilled in the art.

Compounds of Formula (2), wherein V¹═C hereinafter referred to as (2′), may be prepared by cyclocondensation from their precursors of Formula (6) in which LG² has the same definition as depicted above. For example, when LG² is an alkoxy, the reaction involves the presence of a base such as LiHMDS or K₂CO₃ or may be directly obtained without isolation from the previous step under heating conditions.

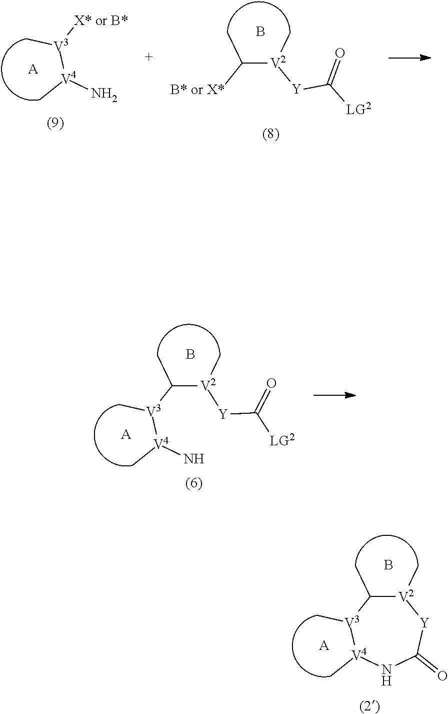

Compounds of Formula (6) may be prepared by a cross-coupling reaction, the “Suzuki reaction” known by the person skilled in the art, from their corresponding precursors of Formula (8) and (9), with the proviso that when (8) bears B*, (9) bears X* or when (8) bears X*, (9) bears B*. B* may be a boronic acid B(OH)₂, or any boronic ester B(OR)₂ such as pinacol boronic ester or a mixture of the two, and X* is a halogen such as Cl, Br or I.

Alternatively, compounds of Formula (2), wherein V¹═C and V²═N, herein after referred to as compounds of formula (2′), may be prepared by cyclisation from their precursors of Formula (6′) in which LG¹ has the same definition as depicted above. For example, when LG¹ is Cl or Br, the reaction involves the presence of a base such as NaH, NaOH, DIEPA or TEA.

Compounds of formula (2′) may also be directly obtained without isolation from the previous step following subsequent cyclization in the Suzuki reaction conditions following heating at high temperature such as 100° C.

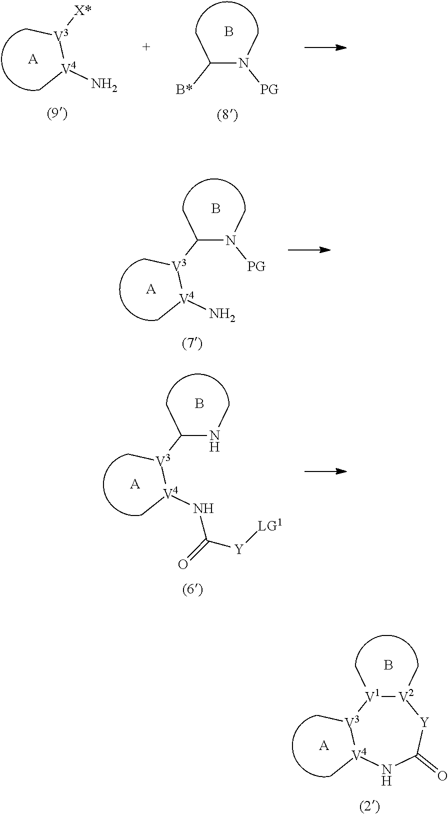

Compounds of Formula (6′) may be prepared by the reaction of the corresponding acyl halide with a base and their precursor (7′) in which the protecting group PG was removed in situ during the reaction. For example, precursors (7′) may be reacted with NaH, DIPEA or TEA and chloroacetyl chloride, methyl bromoacetate or bromopropionyl chloride. Alternatively, PG may be removed using an additional step of deprotection. For example, when PG is tetrahydropyran, deprotection may involve the use of HCl in dioxane.

Compounds of Formula (7′) may be prepared by a cross-coupling reaction, the so-called “Suzuki reaction” known by the person skilled in the art, from their corresponding precursors of Formula (8′) and (9′), with the provisio of when (8′) is bearing B*, (9′) bears X* or when (8′) bears X*, (9′) bears B*. B* and R* have the same definition as previously depicted.

Compounds of Formula (8), (8′), (9) and (9′) are either commercially available, described in the literature or may be prepared by functional group transformations known to the person skilled in the art.

It will be apparent to the person skilled in the art that method analogous to those described above may be used for compounds of formula (I) wherein Q represents Q⁴, Q⁵, or Q⁶.

In another aspect, the present invention provides synthetic intermediates of formula (II),

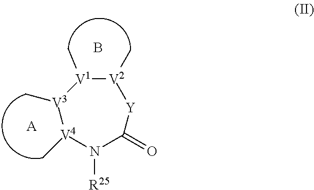

Wherein A, B, Y, V¹, V², V³ and V⁴ are as defined hereabove:
R²⁵ represents hydrogen or CH₂—CO—R^{d}; and
R^{d} represents hydroxy, halogen, amino or C₁₋₄ alkoxy.

In first embodiment, R²⁵ represents CH₂—CO—R^{d}. In a second embodiment, R²⁵ represents hydrogen.

In a first embodiment, A represents A¹. In a second embodiment, A represents A². In a third embodiment, A represents A³. In a fourth embodiment, A represents A⁴. In a fifth embodiment, A represents A⁵. In a sixth embodiment, A represents A⁶. In a seventh embodiment, A represents A⁷.

In a first embodiment, B represents B¹. In a second embodiment, B represents B². In a third embodiment, B represents B³. In a fourth embodiment, B represents B⁴. In a fifth embodiment, B represents B⁵. In a sixth embodiment, B represents B⁶. In a seventh embodiment, B represents B⁷. In an eighth embodiment, B represents B⁸. In yet another aspect, the present invention relates to the use of intermediates of Formula (II) for the synthesis of compounds of formula (I).

### EXPERIMENTAL SECTION

### I. Abbreviations/Recurrent Reagents

ACN or MeCN Acetonitrile
DCM Dichloromethane
EtOAc Ethyl acetate
DMF N,N-Dimethylformamide
DMA Dimethyl acetamide
DMAP Dimethylaminopyridine
EDC 1-Ethyl-3-carbodiimide hydrochloride
MeOH Methanol
DCE Dichloroethane
HATU Hexafluorophosphate Azabenzotriazole Tetramethyl Uronium
HBTU Hexafluorophosphate Benzotriazole Tetramethyl Uronium
HOBt Hydroxybenzotriazole
TCFH Chloro-N,N,N′N′-tetramethylformamidinium hexafluorophosphate
NMI N-Methylimidazole
MTBE or TBME Methyl tert-butyl ether
PTFE polytetrafluoroethylene
ELSD Evaporative light scattering detector
DMSO Dimethylsulfoxide
Brine Saturated aqueous sodium chloride solution
Et₂O Diethyl ether
h Hour
d Days
THF Tetrahydrofuran
AcOH Acetic acid
RT Room temperature
rt Retention time
Rf Retention factor
br Broad
M Molar
MS Mass Spectrometry
[M+H]⁺ Exact mass of protonated ion observed by MS
[M−H]⁻ Exact mass of deprotonated ion observed by MS
mL Milliliter
HPLC High Performance Liquid Chromatography
UPLC Ultra High Performance Liquid Chromatography
LC-MS Liquid Chromatography Mass Spectrometry
ESI Electrospray Ionisation
ES⁺ Electrospray Positive Ionisation
TEA Triethylamine
DIPEA N,N-di-iso-propylethylamine
DEA Diethylamine
CDI Carbonyl diimidazole
PCy3 Tricyclohexylphosphine
TMSCN Trimethylsilyl cyanide
dppf 1,1′-Bis(diphenylphosphino)ferrocene
PEPPSI Pyridine-Enhanced Precatalyst Preparation Stabilization and Initiation
HMDS bis(trimethylsilyl)amide or Hexamethyldisilazane
PPh₃ Triphenylphosphine
AIBN Azobisisobutyronitrile
TFA Trifluoroacetic acid
bs. Broad singlet
NBS N-bromosuccinimide
DME Dimethoxy ethane
HMPA Hexamethylphosphoramide
SFC Supercritical Fluid Chromatography
SCX Strong Cation Exchange HPLC column
TLC Thin Layer Chromatography
Sat. Saturated
Hex Hexane
aq. Aqueous
Eq. Equivalent
min Minute
mmol Millimole
UV Ultraviolet

### Naming Convention:

IUPAC names of chemical reagents, Intermediates and Examples have been generated using Biovia Draw 2020 (version 20.1.100.2161 or 20.1.0.2081). Depending on the Kekule structures of chemical reagents, Intermediates and Examples, Bovia Draw may generate different chemical names. As an illustration, the Kekule structures K1 and K2 are named 4,8,14-triazatricyclo[9.4.0.0^{2,7}]pentadeca-1(11),2,4,6,12,14-hexaen-9-one and 4,8,14-triazatricyclo[9.4.0.0^{2,7}]pentadeca-1(15),2,4,6,11,13-hexaen-9-one, respectively. Both names could be found in the below descriptions.

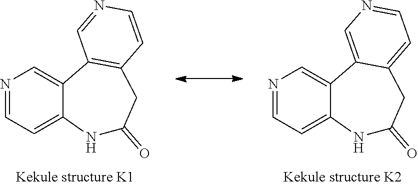 [SMILES: O=C1Cc2ccncc2-c2cnccc2N1 ; O=C1Cc2ccncc2-c2cnccc2N1]

### II. Analytical and Synthetic Methods

All reactions involving air or moisture-sensitive reagents are performed under a nitrogen or argon atmosphere (inert atmosphere) using dried solvents and glassware. Experiments requiring microwave irradiation are performed on a Biotage Initiator Sixty microwave oven upgraded with version 2.0 of the operating software. Experiments are run to reach the required temperature as quickly as possible (maximum irradiation power: 400 W, no external cooling). Commercial solvents and reagents are generally used without further purification, including anhydrous solvents when appropriate (generally Sure-Seal™ products from Aldrich Chemical Company or AcroSeal™ from ACROS Organics). In general, reactions are followed by thin layer chromatography (TLC), high performance liquid chromatography (HPLC) or mass spectrometry (MS) analyses.

NMR spectra were recorded on a Bruker Advance III HD 500 MHz or 400 MHz spectrometer. The chemical shifts (6) reported are given in parts per million (ppm), and the coupling constants (J) are in Hertz (Hz). The spin multiplicities are reported as s=singlet, bs=broad singlet, d=doublet, t=triplet, q=quartet, dd=doublet of doublet, ddd=doublet of doublet of doublet, dt=doublet of triplet, td=triplet of doublet, and m=multiplet.

Mass spectrometric measurements in LC-MS mode are performed as follows:
For acidic elution (Method A1, A1′, A2 and A2′), analyses are performed using a QDA Waters simple quadrupole mass spectrometer. This spectrometer is equipped with an ESI source and an UPLC Acquity Hclass with diode array detector (200 to 400 nm). Data are acquired in a full MS scan from m/z 70 to 800 in positive mode with an acidic elution. The reverse phase separation is carried out at 45° C. on a Waters Acquity UPLC HSS T3 1.8 μm (2.1×50 mm) column for Method A1 and A1′ elution and on a Waters Acquity UPLC HSS T3 1.8 μm (2.1×100 mm) column for Method A2 and A2′. Gradient elution is done with water/ACN/TFA (95/5/0.5 mL/L) (solvent A) and ACN (solvent B) for Method A1 and A2 and Water/Acetonitrile/Formic acid (95/5/(0.05%)) (solvent A) Acetonitrile/Formic acid (99.95/0.05%) (solvent B) for Method A1′ and A2′. Injection volume: 1 μL. Full flow in MS. Gradient Program:

Method A1, A1′

| Time | | B | Flow |
|---|---|---|---|
| (min) | A (%) | (%) | (mL/min) |
| 0 | 99 | 1 | 0.4 |
| 0.3 | 99 | 1 | 0.4 |
| 3.2 | 5 | 95 | 0.4 |
| 3.25 | 5 | 95 | 0.5 |
| 4 | 5 | 95 | 0.5 |
| 4.1 | 99 | 1 | 0.4 |
| 5.5 | 99 | 1 | 0.4 |

Method A2, A2′

| Time | A | B | Flow |
|---|---|---|---|
| (min) | (%) | (%) | (mL/min) |
| 0 | 99 | 1 | 0.4 |
| 0.8 | 99 | 1 | 0.4 |
| 5.3 | 5 | 95 | 0.4 |
| 5.35 | 5 | 95 | 0.5 |
| 7.3 | 5 | 95 | 0.5 |
| 7.35 | 99 | 1 | 0.4 |
| 9 | 99 | 1 | 0.4 |

For acidic elution (Method A3), analysis are performed using a Xevo Waters Q-TOF mass spectrometer. This spectrometer is equipped with an ESI source and a Waters Acquity H-class UPLC with diode array detector (210 to 400 nm). Data are acquired in a full MS scan from m/z 50 to 1200 in positive mode. The reverse phase separation is carried out at 40° C. on an Acquity UPLC HSS T3 C18 column (1.8 μm, 2.1×100 mm). Gradient elution is done with Water/ACN/Formic acid (95/5/750 μL/L) (Solvent C) and Water/ACN/Formic acid (5/95/500 μL/L) (Solvent D) at pH~3. 100% Flow in UV, 10% flow in MS−, 90% flow in ELSD. Injection volume: 0.5 to 2 μL.

### Gradient Program:

Method A3

| Time | C | D | Flow |
|---|---|---|---|
| (min) | (%) | (%) | (mL/min) |
| 0 | 98 | 2 | 0.6 |
| 0.3 | 98 | 2 | 0.6 |
| 5.9 | 5 | 95 | 0.6 |
| 9.3 | 5 | 95 | 0.6 |
| 9.4 | 98 | 2 | 0.6 |
| 14 | 98 | 2 | 0.6 |

For acidic elution (Method A4), analysis is performed using a SYNAPT G2-SI Waters Q-TOF mass spectrometer. This spectrometer is equipped with an ESI source and a Waters Acquity H-class UPLC with diode array detector (210 to 400 nm). Data are acquired in a full MS scan from m/z 50 to 1200 in positive mode. The reverse phase separation is carried out at 45° C. on an Acquity UPLC HSS T3 C18 column (1.8 μm, 2.1×100 mm). Gradient elution is done with Water/ACN/Formic acid (95/5/750 μL/L) (Solvent C) and Water/ACN/Formic acid (5/95/500 μL/L) (Solvent D) pH-3. Full flow in MS. injection volume: 0.5 μL.

### Gradient Program:

Method A4

| Time | C | D | Flow |
|---|---|---|---|
| (min) | (%) | (%) | (mL/min) |
| 0 | 98 | 2 | 0.6 |
| 0.5 | 98 | 2 | 0.6 |
| 5 | 5 | 95 | 0.6 |
| 5.1 | 5 | 95 | 0.6 |
| 7.3 | 5 | 95 | 0.6 |
| 7.5 | 98 | 1 | 0.6 |
| 10 | 98 | 1 | 0.6 |

For acidic elution (Method A5), analyses are performed using a Shimadzu LC-MS 2010EV mass spectrometer for LC-MS analysis. This spectrometer is equipped with an ESI source and HPLC with diode array detector (210 to 400 nm). Data is acquired in a full MS scan from m/z 80 to 2000 in positive mode and negative mode. The reverse phase separation is carried out with Waters X-Select CSH C18 (4.6×150) mm, 3.5 μm column. Column temp: 50° C. Gradient elution is done with Mobile phase with 0.1% Formic acid in water (Phase A) and Acetonitrile (Phase B). Injection volume: 2 μL.

### Gradient Program:

Method A5

| Time | | | Flow |
|---|---|---|---|
| (min) | A (%) | B (%) | (mL/min) |
| 0.01 | 95 | 5 | 1 |
| 1.0 | 95 | 5 | 1 |
| 8.0 | 0 | 100 | 1 |
| 12 | 0 | 100 | 1 |
| 14 | 95 | 5 | 1 |
| 18 | 95 | 5 | 1 |

For acidic elution (Method A6), analyses are performed using a Shimadzu LC-MS 2010EV mass spectrometer for LC-MS analysis. This spectrometer is equipped with an ESI source and HPLC with diode array detector (210 to 400 nm). Data is acquired in a full MS scan from m/z 80 to 2000 in positive mode and negative mode. The reverse phase separation is carried out with Waters X-Select CSH C18 (4.6×150) mm, 3.5 μm column. Column temp: 50° C. Gradient elution is done with Mobile phase with 0.1% TFA in water (Phase A) and Acetonitrile (Phase B). Injection volume: 2 μL.

### Gradient Program:

Method A6

| Time | | | Flow |
|---|---|---|---|
| (min) | A (%) | B (%) | (mL/min) |
| 0.01 | 95 | 5 | 1.2 |
| 1.0 | 95 | 5 | 1.2 |
| 8.0 | 0 | 100 | 1.2 |
| 12 | 0 | 100 | 1.2 |
| 14 | 95 | 5 | 1.2 |
| 18 | 95 | 5 | 1.2 |

For acid elution (Method A7), analyses are performed using an Agilent 1200-6120 LC-MS system coupled to UV detection (254 nM) and MS Detection: Agilent 6120 Mass Spectrometer (ES) m/z 100 to 1000. Column: XSelect CSH C18 XP 130 Å, 2.5 μm, 4.6 mm×30 mm (Waters™). Mobile Phase A: 0.1% Formic acid in water, Mobile Phase B: Acetonitrile+0.1% Formic acid. Flow rate: 2.5 mL/min.

### Gradient Program:

Method A7

| Time | | | Flow |
|---|---|---|---|
| (min) | A(%) | B(%) | (mL/min) |
| 0 | 95 | 5 | 2.5 |
| 3 | 5 | 95 | 2.5 |
| 3.01 | 5 | 95 | 4.5 |
| 3.60 | 5 | 95 | 4.5 |
| 3.7 | 95 | 5 | 4.5 |
| 3.71 | 95 | 5 | 2.5 |
| 4 | 95 | 5 | 2.5 |

For acid elution (Method A9), analyses are performed using similar equipments as above, but the reverse phase separation is carried out with a Waters Cortecs C18 2.7 μm (30×2.1 mm) column. Column temp: 40° C. 1.5 min gradient elution is done with Mobile phase with 0.1% formic acid in water (Phase A) and ACN (Phase B).
For basic elution (Method B1 and B2), analyses are performed using a QDA Waters simple quadrupole mass spectrometer. This spectrometer is equipped with an ESI source and a UPLC Acquity Hclass with diode array detector (200 to 400 nm). Data are acquired in a full MS scan from m/z 70 to 800 in positive mode. The reverse phase separation is carried out at 45° C. on a Waters Acquity UPLC BEHC18 1.7 μm (2.1×50 mm) column for Method B1 and on a Waters Acquity UPLC BEH C18 1.7 μm (2.1×100 mm) column basic elution for Method B2. Gradient elution is performed with water/ACN/ammonium formate (95/5/63 mg/L) (solvent A) and ACN/water/ammonium formate (95/5/(63 mg/L)) (solvent B). Injection volume: 1 μL. Full flow in MS.

### Gradient Program:

Method B1

| Time | | | Flow |
|---|---|---|---|
| (min) | A (%) | B (%) | (mL/min) |
| 0 | 99 | 1 | 0.4 |
| 0.3 | 99 | 1 | 0.4 |
| 3.2 | | | 0.4 |
| 3.25 | 0 | 100 | 0.5 |
| 4 | 0 | 100 | 0.5 |
| 4.1 | 99 | 1 | 0.4 |
| 4.8 | 90 | 1 | 0.4 |

Method B2

| Time | | B | Flow |
|---|---|---|---|
| (min) | A (%) | (%) | (mL/min) |
| 0 | 99 | 1 | 0.4 |
| 0.8 | 99 | 1 | 0.4 |
| 5.30 | 0 | 100 | 0.4 |
| 5.35 | 0 | 100 | 0.5 |
| 7.30 | 0 | 100 | 0.5 |
| 7.35 | 99 | 1 | 0.4 |
| 9 | 90 | 1 | 0.4 |

For Basic elution (Method B3) analysis are performed using a Xevo Waters Q-TOF mass spectrometer. This spectrometer is equipped with an ESI source and a Waters Acquity H-class UPLC with diode array detector (210 to 400 nm). Data are acquired in a full MS scan from m/z 50 to 1200 in positive mode. The reverse phase separation is carried out at 45° C. on an Acquity UPLC BEH C18 column (1.7 μm, 2.1×100 mm). Gradient elution is done with Water/ACN/Ammonium formate (95/5/(40 mg/L ammonium bicarbonate+100 μL/L NH₄OH)) (Solvent A) and ACN (Solvent B) pH-8-9. 100% Flow in UV, 10% flow in MS. 90% Flow in ELSD injection volume: 0.2 to 2 μL.

### Gradient Program:

Method B3

| Time | | | Flow |
|---|---|---|---|
| (min) | A (%) | B (%) | (mL/min) |
| 0 | 98 | 2 | 0.6 |
| 0.3 | 98 | 2 | 0.6 |
| 5.9 | 5 | 95 | 0.6 |
| 9.3 | 5 | 95 | 0.6 |
| 9.4 | 98 | 2 | 0.6 |
| 14 | 98 | 2 | 0.6 |

For basic elution (Method B4) analysis are performed using a SYNAPT G2-SI system and Waters Q-TOF mass spectrometer. This spectrometer is equipped with an ESI source and a Waters Acquity H-class UPLC with diode array detector (210 to 400 nm). Data are acquired in a full MS scan from m/z 50 to 1200 in positive mode. The reverse phase separation is carried out at 45° C. on an Acquity UPLC BEH C18 column (1.7 μm, 2.1×100 mm). Gradient elution is done with water/ACN/ammonium formate (95/5/(63 mg/L+100 μL/L NH₄OH)) (solvent A) and ACN (solvent B), at pH~8-9. Full flow in MS. injection volume: 0.5 μL.

### Gradient Program:

Method B4

| Time | | | Flow |
|---|---|---|---|
| (min) | A(%) | B(%) | (mL/min) |
| 0 | 98 | 2 | 0.6 |
| 0.5 | 98 | 2 | 0.6 |
| 5 | 5 | 95 | 0.6 |
| 5.5 | 5 | 95 | 0.7 |
| 7.3 | 5 | 95 | 0.7 |
| 7.5 | 98 | 1 | 0.6 |
| 10 | 98 | 1 | 0.6 |

For basic elution (Method B5 and B5′), analyses are performed using an Agilent 1200-6120 LC-MS system coupled to UV detection (254 nM) and a MS Detection Agilent 6120 Mass Spectrometer (ES) m/z 100 to 1000. The reverse phase separation is carried out at 45° C. on a XBridge BEH C18 XP Column, 130A, 2.5 μm, 4.6 mm×30 mm (Waters™). Column temp: 40° C. Flow rate: 2.5 mL/min. Gradient elution is done with Mobile phase Acetonitrile/10 mM aqueous ammonium bicarbonate (Phase A) and Acetonitrile (Phase B) for method B5 and Mobile Phase A: 0.1% Ammonia in water, Mobile Phase B: Acetonitrile for method B5′.

### Gradient Program:

Method B5, B5′

| Time | | | Flow |
|---|---|---|---|
| (min) | A(%) | B(%) | (mL/min) |
| 0 | 95 | 5 | 2.5 |
| 3 | 5 | 95 | 2.5 |
| 3.01 | 5 | 95 | 4.5 |
| 3.60 | 5 | 95 | 4.5 |
| 3.7 | 95 | 5 | 4.5 |
| 3.71 | 95 | 5 | 2.5 |
| 4 | 95 | 5 | 2.5 |

For Basic elution (Method B6), analyses are performed using a Shimadzu LC-MS 2010EV mass spectrometer. This spectrometer is equipped with an ESI source and HPLC with diode array detector (210 to 400 nm). Data is acquired in a full MS scan from m/z 80 to 2000 in positive mode and negative mode. The reverse phase separation is carried out with Waters X-Select CSH C18 (4.6×150) mm, 3.5 μm column. Column temp: 50° C. Gradient elution is done with Mobile phase: 10 mM ammonium bicarbonate in Water (Phase A) and acetonitrile (Phase B). Injection volume: 2 μL.

### Gradient Program:

Method B6

| Time | | | Flow |
|---|---|---|---|
| (min) | A (%) | B (%) | (mL/min) |
| 0.01 | 95 | 5 | 1 |
| 1.0 | 95 | 5 | 1 |
| 8.0 | 0 | 100 | 1 |
| 12 | 0 | 100 | 1 |
| 14 | 95 | 5 | 1 |
| 18 | 95 | 5 | 1 |

For Basic elution, (Method B7), analyses are performed using a Shimadzu LC-MS 2010EV mass spectrometer. This spectrometer is equipped with an ESI source and HPLC with diode array detector (210 to 400 nm). Data is acquired in a full MS scan from m/z 80 to 2000 in positive mode and negative mode. The reverse phase separation is carried out with Waters X-Bridge C18 (4.6×150) mm, 5 μm column. Column temp: 50° C. Gradient elution is done with Mobile phase 0.1% Ammonia in Water (Phase A) and Acetonitrile (Phase B). Injection volume: 5 μL.

### Gradient Program:

Method B7

| Time | | | Flow |
|---|---|---|---|
| (min) | A (%) | B (%) | (mL/min) |
| 0.01 | 98 | 2 | 1.2 |
| 6 | 85 | 15 | 1.2 |
| 8 | 85 | 15 | 1.2 |
| 9 | 0 | 100 | 1.2 |
| 12 | 0 | 100 | 1.2 |
| 14 | 98 | 2 | 1.2 |
| 18 | 98 | 2 | 1.2 |

For Basic elution (Method B9), analyses are performed using an Agilent 1200 series LC in tandem with a 6140 mass spectrometer. The reverse phase separation is carried out with a Phenomenex Gemini NX-C18 3 μM (2×20 mm), flow rate 1.0 mL/min, column temperature 40° C., eluting with a 5-95% gradient over 6.0 minutes (solvent A: 10 mM ammonium formate in water+0.1% ammonia solution, solvent B: ACN+5% water+0.1% ammonia solution).
For Basic elution (Method B10), analyses are performed using an Agilent 1290 Infinity II LC in tandem with a 6135 MSD XT mass spectrometer. The reverse phase separation is carried out with an Acquity UPLC BEH C18 2.1×50 mm, 1.7 μM, flow rate 1.5 mL/min, 60° C. column temperature, eluting with a 5-95% gradient over 4.5 minutes (solvent A—10 mM ammonium formate in water+0.1% Ammonia solution, solvent B—ACN+5% water+0.1% ammonia solution).

Analytical chiral LC-MS were all performed at 30° C. on 4.6×150 mm columns with a flow rate of 1.5 mL/min except for Chiralpak IG-u (Daicel) column which dimension is 3×100 mm and the flow rate is 0.425 mL/min. All columns display a granulometry of 3 μm except for WhelkO-1 (R,R) (Regis Technology) which is 3.5 μm and Chiralpak IG-u (Daicel) which is sub-2 μm.

High Resolution Mass spectrometric measurements in LC-MS mode are performed as follows:

A SYNAPT G2-SI Waters Q-TOF mass spectrometer is used for QC analysis. This spectrometer is equipped with an ESI source and a Waters Acquity H-class UPLC with diode array detector (210 to 400 nm). Data are acquired in a full MS scan from m/z 50 to 1200 in positive mode. The reverse phase separation is carried out at 45° C. on an Acquity UPLC BEH C18 column (1.7 μm, 2.1×30 mm). Gradient elution is done with Water/ACN/Formic acid (95/5/(750 μL/L)) (Solvent C) and Water/ACN/Formic acid (5/95/(500 μL/L)) (Solvent D) at pH~3. Full flow in MS. injection volume: 0.5 to 1 μL.

### Gradient Program:

| Time | C | D | Flow |
|---|---|---|---|
| (min) | (%) | (%) | (mL/min) |
| 0 | 95 | 5 | 0.8 |
| 1.8 | 5 | 95 | 0.8 |
| 2.4 | 5 | 95 | 0.8 |
| 2.5 | 95 | 5 | 0.8 |
| 3.1 | 95 | 5 | 0.8 |

Preparative HPLC purifications are performed using SQD Waters or QDa Performance single quadrupole mass spectrometer. This spectrometer is equipped with an ESI source, Waters 2525 binary pump coupled with 2767 sample Manager and with diode array detector (210 to 400 nm). Data are acquired in a full MS scan from m/z 100 to 850 in positive and negative modes. LC parameters: The reverse phase separation is carried out at room temperature on a Waters XBridge OBD MS C18 column (5 μm, 30×50 mm). Typical HPLC flow rate from 35 mL/min to 45 mL/min. Typical example of basic elution: gradient from solvent A (H₂O+10 mM NH₄HCO₃+50 μL/L NH₄OH) and solvent B (100% acetonitrile) [Purification Method P_B]. Typical example of acidic elution: gradient from solvent A (H₂O/TFA: 99.5%/0.5%) and solvent B (ACN/TFA: 99.5%/0.5%) [Purification Method P_A].

Some preparative HPLC purifications are performed using a Gilson Modular System (333 Prep-Scale HPLC Pump (Water), 334 Presp-Scale HPLC Pump (Acetonitrile), 334 Presp-Scale HPLC Pump (Modifier: a solution of 5 mL NH₄OH in 1000 mL of H₂O for basic elution [Purification Method G_B]. or a solution of 20 mL TFA in 1000 mL H₂O [Purification Method G_A]), 171 Diode Array Detector, GX-271 Prep Liquid Handler, PrepFC Fraction Collector) equipped with a YMC Triart-500 g-10 μm-76.5×200 mm column. Typical HPLC flow rate is 180 mL/min.

When analytical methods are not specified in the below protocols, the methods used were similar to the ones described above. It will be apparent to the person skilled in the art that there are analytical and preparative chromatographic methods analogues to the ones described above can be use for the below procedures.

Photochemical reactions are performed with a Photoreactor M1 or M2 (from Penn PHD) mentioned as Pennoc in the following experimental protocols.

### III. Intermediates

### Intermediate N1: 9,10-dimethyl-5,7-dihydropyrido[3,2-d][1]benzazepin-6-one

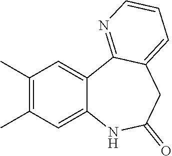 [SMILES: Cc1cc2c(cc1C)-c1ncccc1CC(=O)N2]

### Step 1: Synthesis of 1-bromo-4,5-dimethyl-2-nitrobenzene N1_1

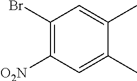

A stirred solution of 4,5-dimethyl-2-nitro-aniline (5.00 g, 30.1 mmol) in 47% aqueous HBr (60 mL) was heated at 70° C. for 1.5 h. The reaction mixture was cooled at 0° C. followed by dropwise addition of a solution of NaNO₂ (16.0 g, 232 mmol) in water (60 mL) over 20 min and the reaction mixture was stirred at 0° C. for 30 min. At 0° C., a solution of CuBr (51.8 g, 361 mmol) in 47% aqueous HBr (150 mL) was slowly added over a period of 15 min and the reaction mixture was allowed to warm at room temperature before being heated at 70° C. for 30 min. After cooling to room temperature, the reaction mixture was extracted with DCM (3×150 mL). The organic layer was separated, washed with 2N aqueous NaOH (250 mL), dried over anhydrous Na₂SO₄ and concentrated under vacuum to afford the title compound (5.20 g, yield: 75%) as a brown solid. This compound was taken to the next step without purification. ¹H NMR (400 MHz, DMSO-d₆) δ 2.26 (s, 3H), 2.30 (s, 3H), 7.71 (s, 1H), 7.86 (s, 1H).

### Step 2: Synthesis of 2-(4,5-dimethyl-2-nitrophenyl)-4,4,5,5-tetramethyl-1,3,2-dioxaborolane N1_2

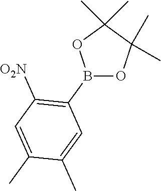

To a solution of 1-bromo-4,5-dimethyl-2-nitrobenzene (Intermediate N1_1, 3.00 g, 13.0 mmol) in dioxane (30 mL) were added bispinacolatodiboron (4.97 g, 19.6 mmol) and potassium acetate (3.33 g, 33.9 mmol) and the reaction mixture was purged with argon for 20 min at room temperature. PdCl₂(dppf (0.48 g, 0.65 mmol) was added and the reaction mixture was heated at 80° C. for 16h. After cooling to room temperature, the reaction mixture was concentrated under vacuum and the crude was purified by column chromatography on silica gel (using DCM as eluent) to afford the title compound (1.80 g, yield: 50%) as an off-white solid. ¹H NMR (400 MHz, DMSO-d₆) δ 1.32 (s, 12H), 2.33 (s, 6H), 7.36 (s, 1H), 7.98 (s, 1H).

### Step 3: Synthesis of 4,5-dimethyl-2-(4,4,5,5-tetramethyl-1,3,2-dioxaborolan-2-yl)aniline N1_3

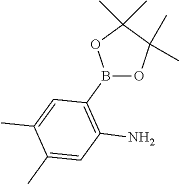

To a suspension of 10% Pd/C (50% wet, 0.20 g) in MeOH (30 mL) was added 2-(4,5-dimethyl-2-nitrophenyl)-4,4,5,5-tetramethyl-1,3,2-dioxaborolane (Intermediate N1_2, 1.00 g, 3.61 mmol) and the reaction mixture was stirred at room temperature for 5 h under hydrogen atmosphere (P=1 atm). After completion, the reaction mixture was filtered through a pad of Celite®, washed with MeOH (100 mL) and the filtrate was concentrated under vacuum to afford the title compound (0.515 g, yield: 58%) as an off-white solid. This compound was taken to the next reaction without purification. ¹H NMR (400 MHz, DMSO-d₆) δ 1.26 (s, 12H), 2.03 (s, 3H), 2.08 (s, 3H), 5.18 (s, 2H), 6.38 (s, 1H), 7.10 (s, 1H).

### Step 4: Synthesis of 3-(bromomethyl)-2-chloropyridine N1_4

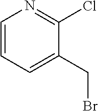

Under nitrogen atmosphere, to a solution of 2-chloro-3-methyl-pyridine (8.6 mL, 78.4 mmol) in DCE (200 mL) were added NBS (16.7 g, 94.1 mmol) and AIBN (1.29 g, 7.84 mmol) and the reaction mixture was heated at 70° C. for 16 h. After cooling to room temperature, the reaction mixture was treated with water (50 mL) and extracted with DCM (2×100 mL). The organic layer was separated, washed with brine (60 mL), dried over anhydrous Na₂SO₄ and concentrated under vacuum. Purification by column chromatography on silica gel (using 3% EtOAc in hexanes as eluent) afforded the title compound (8.0 g, yield: 49%) as a colorless oil. LC-MS (Method B6): [M+H]⁺ m/z: 207, rt: 1.74 min, purity: 87%. ¹H NMR (400 MHz, DMSO-d₆) δ 4.78 (s, 2H), 7.46-7.49 (m, 1H), 8.17 (d, J=7.6 Hz, 1H), 8.41 (d, J=4.4 Hz, 1H).

### Step 5: Synthesis of 2-(2-chloropyridin-3-yl)acetonitrile N1_5

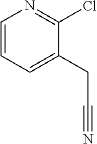

At 0° C., to a solution of 3-(bromomethyl)-2-chloropyridine (Intermediate N1_4, 8.00 g, 38.7 mmol) in CH₃CN (100 mL) were added a 1 M solution of tetrabutylammonium fluoride in THF (50.4 mL, 50.4 mmol) and TMSCN (14.5 mL, 116 mmol) and the reaction mixture was stirred at room temperature for 3 h. After completion, the reaction mixture was treated with saturated aqueous NaHCO₃ solution (70 mL) and extracted with DCM (3×100 mL). The organic layer was separated, dried over anhydrous Na₂SO₄ and concentrated under vacuum. Purification by column chromatography on silica gel (using 10% EtOAc in hexanes as eluent) afforded the title compound (3.10 g, yield: 52%) as a white solid. LC-MS (Method A5): [M+H]⁺ m/z: 152.9, rt: 1.48 min, purity: 96%. ¹H NMR (400 MHz, DMSO-d₆) δ 4.14 (s, 2H), 7.48-7.51 (m, 1H), 7.99 (d, J=7.6 Hz, 1H), 8.41 (d, J=5.2 Hz, 1H).

### Step 6: Synthesis of 2-(2-chloropyridin-3-yl)acetic acid N1_6

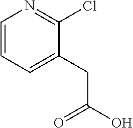 [SMILES: O=C(O)Cc1cccnc1Cl]

A suspension of 2-(2-chloropyridin-3-yl)acetonitrile (Intermediate N1_5, 3.10 g, 20.3 mmol) in a 15% aqueous NaOH solution (51.7 mL, 194 mmol) was heated at 90° C. for 5 h. After cooling to room temperature, the reaction mixture was acidified with concentrated aqueous HCl (80 mL) up to pH=1 and left at room temperature for 1 h. The resulting precipitate was collected by filtration and washed with pentane (3×60 mL) to afford the title compound (2.60 g, yield: 75%) as a white solid. This compound was taken to the next reaction without purification. LC-MS (Method B6): [M+H]⁺ m/z: 171.7, rt: 0.69 min, purity: 98%. ¹H NMR (400 MHz, DMSO-d₆) δ 3.76 (s, 2H), 7.39-7.42 (m, 1H), 7.85 (d, J=7.2 Hz, 1H), 8.27 (d, J=4.8 Hz, 1H), 12.61 (s, 1H).

### Step 7: Synthesis of ethyl 2-(2-chloropyridin-3-yl)acetate N1_7

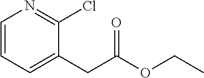

To a solution of 2-(2-chloropyridin-3-yl)acetic acid (Intermediate N1_6, 2.60 g, 15.2 mmol) in absolute ethanol (50 mL) was added concentrated sulfuric acid (6.1 mL, 114 mmol) at room temperature and the reaction mixture was heated at 70° C. for 16 h. After cooling to room temperature, the reaction mixture was basified with saturated NaHCO₃ solution (80 mL) up to pH=9 and the solvent was removed under vacuum. The aqueous layer was extracted with DCM (2×100 mL). The organic layer was separated, dried over anhydrous Na₂SO₄ and concentrated under vacuum. Purification by column chromatography on silica gel (using 5% EtOAc in hexanes as eluent) afforded the title compound (2.80 g, yield: 93%) as a pale yellow oil. LC-MS (Method A5): [M+H]⁺ m/z: 199.8, rt: 1.74 min, purity: 98%. ¹H NMR (400 MHz, CDCl₃) δ 1.25-1.28 (m, 3H), 3.82 (s, 2H), 4.16-4.24 (m, 2H), 7.22-7.30 (m, 1H), 7.64 (d, J=7.2 Hz, 1H), 8.38 (d, J=4.8 Hz, 1H).

### Step 8: Synthesis of 9,10-dimethyl-5,7-dihydropyrido[3,2-d][1]benzazepin-6-one N1

A suspension of 4,5-dimethyl-2-(4,4,5,5-tetramethyl-1,3,2-dioxaborolan-2-yl)aniline (Intermediate N1_3, 0.87 g, 3.51 mmol), ethyl 2-(2-chloropyridin-3-yl)acetate (Intermediate N1_7, 0.70 g, 3.51 mmol) and K₂CO₃ (0.97 g, 7.01 mmol) in dioxane (16 mL) and H₂O (4 mL) was purged with argon for 30 min at room temperature. Pd(PPh₃)₄ (0.20 g, 0.18 mmol) was added and the reaction mixture was heated at 100° C. for 16 h. After cooling to room temperature, the reaction mixture was treated with water (25 mL) and the resulting precipitate was collected by filtration and dried under vacuum. Purification by column chromatography on silica gel (using 3% MeOH in DCM as eluent) afforded the title compound (0.335 g, yield: 40%) as an off-white solid. LC-MS (Method B7): [M+H]⁺ m/z: 239.0, rt: 2.01 min, purity: 98%. ¹H NMR (400 MHz, DMSO-d₆) δ 2.27 (s, 3H), 2.38 (s, 3H), 3.32 (s, 2H), 6.96 (s, 1H), 7.39-7.42 (m, 1H), 7.75 (s, 1H), 7.80 (d, J=7.6 Hz, 1H), 8.63 (d, J=4.4 Hz, 1H), 10.02 (s, 1H).

### Intermediate N2: 9,10-dimethyl-5,7-dihydropyrido[4,3-d][1]benzazepin-6-one

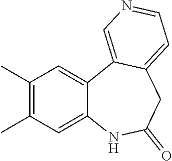 [SMILES: Cc1cc2c(cc1C)-c1cnccc1CC(=O)N2]

### Step 1: Synthesis of (E)-3-bromo-4-(2-(methylsulfinyl)-2-(methylthio)vinyl)pyridine N2_1

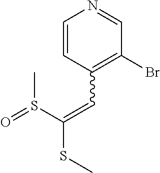

To a solution of 3-bromopyridine-4-carbaldehyde (10.0 g, 53.8 mmol) in a 1:1 mixture of THF and MeOH (120 mL) was added methylsulfanyl(methylsulfinyl)methane (11.1 mL, 108 mmol) and the reaction mixture was stirred at room temperature for 10 min. A 40% solution of benzyltrimethylammonium hydroxide in methanol (45 mL, 108 mmol) was added dropwise and the reaction mixture was heated at 70° C. for 16 h under nitrogen atmosphere. After cooling to room temperature, the reaction mixture was concentrated under vacuum and the residue was partitioned between DCM (500 mL) and water (500 mL). The organic layer was separated, washed with water (500 mL), dried over anhydrous Na₂SO₄ and concentrated under vacuum. Purification by column chromatography on silica gel (using 20% EtOAc in hexanes as eluent) afforded the title compound (7.50 g, yield: 48%) as a pale-yellow oil. ¹H NMR (400 MHz, CDCl₃) δ 3.30 (s, 3H), 3.34 (s, 3H), 7.70 (d, J=4.9 Hz, 1H), 8.47 (d, J=4.4 Hz, 1H), 8.72 (s, 1H). 1H merged in solvent peak.

### Step 2: Synthesis of methyl 2-(3-bromopyridin-4-yl)acetate N2_2

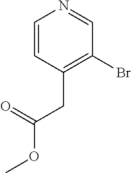

To a solution of (E)-3-bromo-4-(2-(methylsulfinyl)-2-(methylthio)vinyl)pyridine (Intermediate N2_1, 5.00 g, 17.1 mmol) in MeOH (50 mL) was added a 4 M solution of HCl in MeOH (50 mL) and the reaction mixture was heated at 70° C. for 16 h. After cooling to room temperature, the reaction mixture was concentrated under vacuum and the residue was treated with H₂O (50 mL) and diluted with DCM (150 mL). The organic layer was separated, washed with H₂O (50 mL), dried over anhydrous Na₂SO₄ and concentrated under vacuum. Purification by column chromatography on silica gel (using 5% MeOH in DCM as eluent) afforded the title compound (2.90 g, yield: 74%) as a brown oil. LC-MS (Method B6): [M+H]⁺ m/z: 231.8, rt: 1.58 min, purity: 78%. ¹H NMR (400 MHz, DMSO-d₆) δ 3.64 (s, 3H), 3.94 (s, 2H), 7.49 (d, J=4.4 Hz, 1H), 8.56 (d, J=4.9 Hz, 1H), 8.77 (s, 1H).

### Step 3: Synthesis of methyl 2-(3-(4,5-dimethyl-2-nitrophenyl)pyridin-4-yl)acetate N2_3

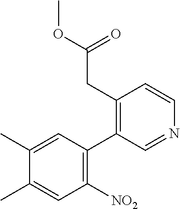 [SMILES: COC(=O)Cc1ccncc1-c1cc(C)c(C)cc1[N+](=O)[O-]]

To a solution of methyl 2-(3-bromopyridin-4-yl)acetate (Intermediate N2_2, 2.00 g, 8.69 mmol) in dry toluene (50 mL) were added 2-(4,5-dimethyl-2-nitrophenyl)-4,4,5,5-tetramethyl-1,3,2-dioxaborolane (Intermediate N1_2, 2.41 g, 8.69 mmol), K₂CO₃ (1.80 g, 13.0 mmol), TEA (2.23 mL, 17.4 mmol) and PPh₃ (0.23 g, 0.87 mmol) and the reaction mixture was purged with nitrogen for 30 min. Pd(OAc)₂ (0.10 g, 0.44 mmol) was added and the reaction mixture was again purged with nitrogen for 10 min before being heated at 100° C. for 16 h. After cooling to room temperature, the reaction mixture was concentrated under vacuum. The residue was treated with H₂O (100 mL) and diluted with EtOAc (100 mL). The organic layer was separated, washed with H₂O (100 mL), dried over anhydrous Na₂SO₄ and concentrated under vacuum. Purification by column chromatography on silica gel (using 10% EtOAc in hexanes as eluent) afforded the title compound (0.752 g, yield: 29%) as an off-white solid. LC-MS (Method A5): [M+H]⁺ m/z: 301.0, rt: 1.95 min, purity: 90%. ¹H NMR (400 MHz, CDCl₃) δ 2.38 (s, 3H), 2.43 (s, 3H), 3.37-3.44 (m, 1H), 3.47-3.54 (m, 1H), 3.62 (s, 3H), 7.09 (s, 1H), 7.37 (d, J=4.9 Hz, 1H), 7.97 (s, 1H), 8.37 (s, 1H), 8.61 (d, J=4.4 Hz, 1H).

### Step 4: Synthesis of methyl 2-(3-(2-amino-4,5-dimethylphenyl)pyridin-4-yl)acetate and 9,10-dimethyl-5,7-dihydro-6H-benzo[b]pyrido[3,4-d]azepin-6-one N2_4

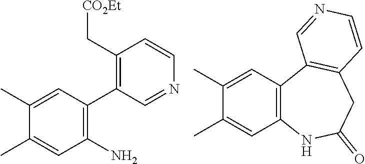

To a solution of methyl 2-(3-(4,5-dimethyl-2-nitrophenyl)pyridin-4-yl)acetate (Intermediate N2_3, 0.72 g, 2.40 mmol) in MeOH (30 mL) was added 20% Pd/C (0.20 g) and the reaction mixture was stirred at room temperature for 3 h under hydrogen atmosphere (P=1 atm). After completion, the reaction mixture was filtered through a pad of Celite®, washed with MeOH (50 mL) and the filtrate was concentrated under vacuum to afford a mixture of methyl 2-(3-(2-amino-4,5-dimethylphenyl)pyridin-4-yl)acetate and 9,10-dimethyl-5,7-dihydro-6H-benzo[b]pyrido[3,4-d]azepin-6-one (0.60 g) as an off-white solid. This mixture was taken to the next reaction without purification. LC-MS (Method A5): [M+H]⁺ m/z: 238.9, rt: 1.71 min, purity: 91%. LC-MS (Method A5): [M+H]⁺ m/z: 271.1, rt: 1.84 min, purity: 4%.

### Step 5: Synthesis of 9,10-dimethyl-5,7-dihydropyrido[4,3-d][1]benzazepin-6-one N2

A suspension of methyl 2-(3-(2-amino-4,5-dimethylphenyl)pyridin-4-yl)acetate and 9,10-dimethyl-5,7-dihydro-6H-benzo[b]pyrido[3,4-d]azepin-6-one mixture (Intermediate N2_4, 0.60 g, 2.22 mmol) and K₂CO₃ (0.92 g, 6.66 mmol) in EtOH (25 mL) was heated at 80° C. for 16 h. After cooling to room temperature, the reaction mixture was treated with H₂O (100 mL) and diluted with DCM (150 mL). The organic layer was separated, washed with H₂O (150 mL), dried over anhydrous Na₂SO₄ and concentrated under vacuum. Purification by trituration with Et₂O (25 mL) afforded the title compound (0.48 g, yield: 80%) as an off-white solid. LC-MS (Method A5): [M+H]⁺ m/z: 238.9, rt: 1.64 min, purity: 98%. ¹H NMR (400 MHz, DMSO-d₆) δ 2.27 (s, 3H), 2.29 (s, 3H), 3.41 (s, 2H), 7.00 (s, 1H), 7.43 (d, J=4.5 Hz, 1H), 7.51 (s, 1H), 8.55 (d, J=5.0 Hz, 1H), 8.77 (s, 1H), 10.06 (s, 1H).

### Intermediate N3: 9,10-dimethyl-5,7-dihydropyrido[2,3-d][1]benzazepin-6-one

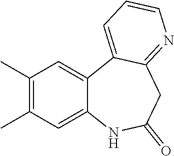 [SMILES: Cc1cc2c(cc1C)-c1cccnc1CC(=O)N2]

### Step 1: Synthesis of ethyl 2-(3-bromo-2-pyridyl)acetate N3_1

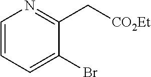 [SMILES: CCOC(=O)Cc1ncccc1Br]

At −78° C., to a solution of 3-bromo-2-methyl-pyridine (5.00 g, 29.1 mmol) in dry THF (100 mL) was added LiHMDS (1 M solution in THF, 58 mL, 58.0 mmol) and the reaction mixture was stirred at 0° C. for 1 h. Diethyl carbonate (5.15 g, 43.6 mmol) was added at 0° C. and the reaction mixture was stirred at room temperature for 15 min. The reaction mixture was treated with H₂O (200 mL) and extracted with EtOAc (2×200 mL). The combined organic layer was dried over anhydrous Na₂SO₄ and concentrated under vacuum. Purification by column chromatography on silica gel (using DCM as eluent) afforded the title compound (5.00 g, yield: 71%) as a brown oil. LC-MS (Method B6): [M+H]⁺ m/z: 243.8, rt: 1.71 min, purity: 94%. ¹H NMR (400 MHz, DMSO-d₆) δ 1.18 (t, J=6.8 Hz, 3H), 3.98 (s, 2H), 4.11 (q, J=6.8 Hz, 2H), 7.27-7.30 (m, 1H), 8.08 (d, J=8.4 Hz, 1H), 8.50 (d, J=4.4 Hz, 1H).

### Step 2: Synthesis of ethyl 2-(3-(4,5-dimethyl-2-nitrophenyl)pyridin-2-yl)acetate N3_2

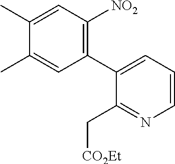 [SMILES: CCOC(=O)Cc1ncccc1-c1cc(C)c(C)cc1[N+](=O)[O-]]

To a solution of ethyl 2-(3-bromo-2-pyridyl)acetate (Intermediate N3_1, 2.00 g, 8.19 mmol) in dioxane (40 mL) were added 2-(4,5-dimethyl-2-nitrophenyl)-4,4,5,5-tetramethyl-1,3,2-dioxaborolane (Intermediate N1_2, 2.73 g, 9.83 mmol) and K₃PO₄ (3.48 g, 16.4 mmol) and the reaction mixture was purged with argon for 20 min. PdCl₂(dppf) (0.30 g, 0.41 mmol) was added and the reaction mixture was heated at 80° C. for 16 h. After cooling to room temperature, the reaction mixture was concentrated under vacuum. Purification by column chromatography on silica gel (using 15% EtOAc in hexanes as eluent) afforded the title compound (1.20 g, yield: 47%) as an off-white solid. LC-MS (Method B6): [M+H]⁺ m/z: 315.1, rt: 1.97 min, purity: 99%. ¹H NMR (400 MHz, DMSO-d₆) δ 1.04 (t, J=6.8 Hz, 3H), 2.31 (s, 3H), 2.36 (s, 3H), 3.46-3.59 (m, 2H), 3.91 (q, J=6.8 Hz, 2H), 7.18 (s, 1H), 7.34-7.37 (m, 1H), 7.58 (d, J=7.6 Hz, 1H), 8.00 (s, 1H) 8.52 (d, J=5.2 Hz, 1H).

### Step 3: Synthesis of ethyl 2-(3-(2-amino-4,5-dimethylphenyl)pyridin-2-yl)acetate N3_3

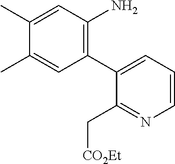 [SMILES: CCOC(=O)Cc1ncccc1-c1cc(C)c(C)cc1N]

To a solution of ethyl 2-(3-(4,5-dimethyl-2-nitrophenyl)pyridin-2-yl)acetate (Intermediate N3_2, 1.10 g, 3.50 mmol) in MeOH (25 mL) was added 10% Pd/C (0.20 g) and the reaction mixture was stirred at room temperature for 4 h under hydrogen atmosphere (P=1 atm). After completion, the reaction mixture was filtered through a pad of Celite®, washed with MeOH (50 mL) and the filtrate was concentrated under vacuum to afford the title compound (0.81 g, yield: 81%) as a colorless oil. This compound was taken to the next reaction without purification. LC-MS (Method A5): [M+H]⁺ m/z: 285.0, rt: 1.93 min, purity: 31%. ¹H NMR (400 MHz, DMSO-d₆) δ 1.07 (t, J=7.4 Hz, 3H), 2.06 (s, 3H), 2.12 (s, 3H), 3.62 (s, 2H), 3.94 (q, J=7.4 Hz, 2H), 4.34 (s, 2H), 6.56 (s, 1H), 6.60 (s, 1H), 7.33-7.36 (m, 1H), 7.53 (d, J=7.6 Hz, 1H), 8.48 (d, J=5.2 Hz, 1H).

### Step 4: Synthesis of 9,10-dimethyl-5,7-dihydropyrido[2,3-d][1]benzazepin-6-one N3

A suspension of ethyl 2-(3-(2-amino-4,5-dimethylphenyl)pyridin-2-yl)acetate (Intermediate N3_3, 0.80 g, 2.81 mmol) and K₂CO₃ (1.17 g, 8.44 mmol) in absolute EtOH (30 mL) was stirred at room temperature for 16 h. After completion, the reaction mixture was treated with H₂O (50 mL) and the resulting precipitate was collected by filtration, washed with Et₂O (50 mL) and n-pentane (50 mL) and dried under vacuum to afford the title compound (0.44 g, yield: 66%) as an off-white solid. LC-MS (Method B7): [M+H]⁺ m/z: 239.0, rt: 2.10 min, purity: 99%. ¹H NMR (400 MHz, DMSO-d₆) δ 2.26 (s, 3H), 2.28 (s, 3H), 3.55 (s, 2H), 7.00 (s, 1H), 7.44-7.48 (m, 2H), 8.01 (d, J=8.0 Hz, 1H), 8.53 (d, J=4.8 Hz, 1H), 10.09 (bs, 1H).

### Intermediate N4: 3,9,10-trimethyl-5,7-dihydropyrido[2,3-d][1]benzazepin-6-one

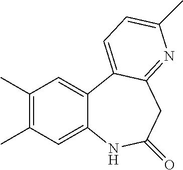

### Step 1: Synthesis of 2-(3-bromo-6-methylpyridin-2-yl)acetonitrile N4_1

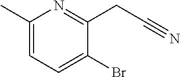

At 0° C., to a solution of 3-bromo-2-fluoro-6-methyl-pyridine (4.3 mL, 26.3 mmol) in dry CH₃CN (5.50 mL, 105 mmol) and in dry toluene (50 mL) was added KHMDS (1 M solution in THF, 31.5 mL, 31.5 mmol) and the reaction mixture was stirred at 0° C. for 30 min, then allowed to reach room temperature for 16 h. The reaction mixture was diluted with EtOAc (100 mL) and washed with a 2 N aqueous HCl solution (2×40 mL), brine (2×40 mL), dried over anhydrous Na₂SO₄ and concentrated under vacuum. Purification by column chromatography on silica gel (using 5% EtOAc in hexanes as eluent) afforded the title compound (3.20 g, yield: 58%) as a yellow solid. LC-MS (Method A5): [M+H]⁺ m/z: 210.8, rt: 1.70 min, purity: 53%. ¹H NMR (400 MHz, CDCl₃) δ 2.53 (s, 3H), 4.02 (s, 2H), 7.02 (d, J=8.0 Hz, 1H), 7.72 (d, J=8.0 Hz, 1H).

### Step 2: Synthesis of methyl 2-(3-bromo-6-methylpyridin-2-yl)acetate N4_2

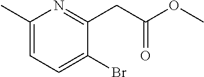

Under nitrogen atmosphere, a solution of 2-(3-bromo-6-methylpyridin-2-yl)acetonitrile (Intermediate N4_1, 2.00 g, 9.48 mmol) in a 4 M HCl solution in MeOH (30 mL) was heated at 70° C. for 16 h. After cooling to room temperature, the reaction mixture was basified with saturated aqueous NaHCO₃ solution (80 mL) up to pH=9 and MeOH was removed under vacuum. The aqueous layer was extracted with DCM (2×100 mL). The organic layer was separated, dried over anhydrous Na₂SO₄ and concentrated under vacuum. Purification by column chromatography on silica gel (using 5% EtOAc in hexanes as eluent) afforded the title compound (1.20 g, yield: 52%) as a pale yellow oil. LC-MS (Method A6): [M+H]⁺ m/z: 244.1, rt: 1.14 min, purity: 65%. ¹H NMR (400 MHz, DMSO-d₆) δ 2.41 (s, 3H), 3.63 (s, 3H), 3.94 (s, 2H), 7.14 (d, J=8.0 Hz, 1H), 7.93 (d, J=8.0 Hz, 1H).

### Step 3: Synthesis of 3,9,10-trimethyl-5,7-dihydropyrido[2,3-d][1]benzazepin-6-one N4

A stirred solution of 2-(3-bromo-6-methylpyridin-2-yl)acetate (Intermediate N4_2, 0.60 g, 2.43 mmol), 4,5-dimethyl-2-(4,4,5,5-tetramethyl-1,3,2-dioxaborolan-2-yl)aniline (Intermediate N1_3, 0.60 g, 2.43 mmol) and K₂CO₃ (0.68 g, 4.92 mmol) in dioxane (16 mL) and H₂O (4 mL) was purged with argon for 30 min at room temperature. Pd(PPh₃)₄ (0.14 g, 0.12 mmol) was added and the reaction mixture was heated in a sealed tube at 100° C. for 16 h. After cooling to room temperature, the reaction mixture was treated with H₂O (25 mL) and the resulting precipitate was collected by filtration, washed with pentane (2×15 mL) and dried by suction. Purification by column chromatography on silica gel (using 2% MeOH in DCM as eluent) afforded the title compound (0.28 g, yield: 45%) as an off-white solid. LC-MS (Method B7): [M+H]⁺ m/z: 253.0, rt: 2.28 min, purity: 99%. ¹H NMR (400 MHz, DMSO-d₆) δ 2.25 (s, 3H), 2.27 (s, 3H), 3.49 (s, 2H), 6.98 (s, 1H), 7.30 (d, J=8.0 Hz, 1H), 7.40 (s, 1H), 7.88 (d, J=8.0 Hz, 1H), 10.04 (s, 1H). 3H protons are merged with solvent peak.

### Intermediate N5: 8,9-dimethyl-4,6-dihydropyrazolo[1,5-a][1,5]benzodiazepin-5-one

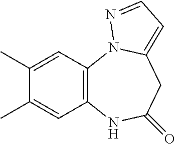 [SMILES: Cc1cc2c(cc1C)-n1nccc1CC(=O)N2]

### Step 1: Synthesis of 3-(benzyloxy)propanoic acid N5_1

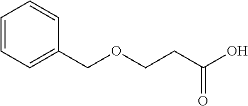 [SMILES: O=C(O)CCOCc1ccccc1]

At 0° C., to a solution of 3-benzyloxypropan-1-ol (5.00 g, 30.1 mmol) in acetone (50 mL) was added Jones reagent (2.5 M solution in H₂O, 16.2 mL, 40.6 mmol) dropwise and the reaction mixture was stirred at room temperature for 30 min. After completion, the reaction mixture was filtered through a pad of Celite® and the filtrate was concentrated under vacuum. The residue was diluted with H₂O (100 mL) and extracted with EtOAc (2×100 mL). The organic layer was separated, dried over anhydrous Na₂SO₄ and concentrated under vacuum. Purification by column chromatography on silica gel (using 2 to 3% MeOH in DCM as eluent) afforded the title compound (3.00 g, yield: 55%) as a colorless oil.

¹H NMR (400 MHz, CDCl₃) δ 2.68 (t, J=6.3 Hz, 2H), 3.76 (t, J=6.3 Hz, 2H), 4.56 (s, 2H), 7.29-7.37 (m, 5H). One H proton merged in the solvent peak.

### Step 2: Synthesis of 3-(benzyloxy)-N-methoxy-N-methylpropanamide N5_2

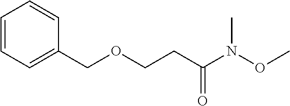 [SMILES: CON(C)C(=O)CCOCc1ccccc1]

To a solution of 3-(benzyloxy)propanoic acid (Intermediate N5_1, 3.00 g, 16.6 mmol) in dry CH₃CN (60 mL) were successively added O,N-dimethylhydroxylamine hydrochloride (1.95 g, 20.0 mmol), NMI (2.7 mL, 33.3 mmol) and TCFH (7.00 g, 25.0 mmol) and the reaction mixture was stirred at room temperature for 16 h. After completion, the reaction mixture was treated with H₂O (100 mL) and extracted with EtOAc (2×200 mL). The organic layer was separated, dried over anhydrous Na₂SO₄ and concentrated under vacuum. Purification by column chromatography on silica gel (using 3 to 5% MeOH in DCM as eluent) afforded the title compound (3.20 g, yield: 86%) as a colorless oil. LC-MS (Method A5): [M+H]⁺ m/z: 224.0, rt: 0.78 min, purity: 98%.

¹H NMR (400 MHz, CDCl₃) δ 2.76-2.81 (m, 2H), 3.21 (s, 3H), 3.70 (s, 3H), 3.82 (t, J=6.4 Hz, 2H), 4.56 (s, 2H) 7.34-7.36 (m, 5H).

### Step 3: Synthesis of (E)-5-(benzyloxy)-1-(methoxy(methyl)amino)pent-1-en-3-one N5_3

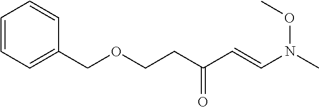 [SMILES: CON(C)/C=C/C(=O)CCOCc1ccccc1]

A solution of 3-(benzyloxy)-N-methoxy-N-methylpropanamide (Intermediate N5_2, 3.20 g, 14.3 mmol) and acetylenemagnesium chloride (0.50 M solution in THF, 36 mL, 17.9 mmol) in dry THE (65 mL) was heated at 50° C. for 40 min. The reaction mixture was cooled at 30° C. followed by addition of saturated aqueous NH₄Cl (35 mL) and heated at 50° C. for 40 min. After cooling to room temperature, the reaction mixture was treated with H₂O (100 mL) and extracted with EtOAc (2×100 mL). The organic layer was separated, dried over anhydrous Na₂SO₄ and concentrated under vacuum. Purification by column chromatography on silica gel (using 2 to 3% MeOH in DCM as eluent) afforded the title compound (2.70 g, yield: 76%) as a yellow oil.

LC-MS (Method A5): [M+H]⁺ m/z: 250.0, rt: 1.76 min, purity: 84%. ¹H NMR (400 MHz, CDCl₃) δ 2.73 (t, J=6.7 Hz, 2H), 3.14 (s, 3H), 3.67 (s, 3H), 3.81 (t, J=6.7 Hz, 2H), 4.55 (s, 2H), 5.48 (d, J=12.6 Hz, 1H), 7.29-7.37 (m, 5H), 7.41 (d, J=12.6 Hz, 1H).

### Step 4: Synthesis of (4,5-dimethyl-2-nitrophenyl)hydrazine, hydrochloride N5_4

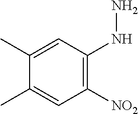

At 10° C., to a solution of 4,5-dimethyl-2-nitro-aniline (5.00 g, 30.1 mmol) in concentrated aqueous HCl (38 mL) was added dropwise a solution of sodium nitrite (2.08 g, 30.1 mmol) in H₂O (20 mL). The reaction mixture was poured into a solution of tin chloride monohydrate (13.6 g, 60.2 mmol) in concentrated aqueous HCl (15 mL) at 0° C. and the reaction mixture was stirred at room temperature for 1 h. After completion, the resulting precipitate was collected by filtration and dried under vacuum to afford the title compound (5.00 g, crude) as a yellow solid. LC-MS (Method A6): [M+H]⁺ m/z: 182.0, rt: 1.59 min, purity: 92%. ¹H NMR (400 MHz, DMSO-d₆) δ 2.23 (s, 3H), 2.30 (s, 3H), 7.18 (s, 1H), 7.95 (s, 1H), 9.10 (brs, 1H). 2H protons are merged in the solvent peak.

### Step 5: Synthesis of 5-(2-(benzyloxy)ethyl)-1-(4,5-dimethyl-2-nitrophenyl)-1H-pyrazole N5_5

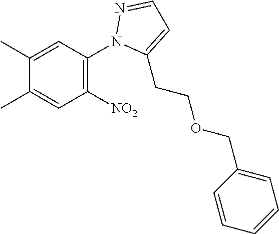 [SMILES: Cc1cc(-n2nccc2CCOCc2ccccc2)c([N+](=O)[O-])cc1C]

A solution of (E)-5-(benzyloxy)-1-(methoxy(methyl)amino)pent-1-en-3-one (Intermediate N5_3, 2.60 g, 10.4 mmol), 4,5-dimethyl-2-nitrophenyl)hydrazine, hydrochloride (Intermediate N5_4, 2.84 g, 13.0 mmol) and Na₂CO₃ (2.21 g, 20.9 mmol) in MeOH (40 mL) and water (6.5 mL) was heated at reflux for 2 h. After cooling to room temperature, the reaction mixture was concentrated under vacuum. The residue was diluted with water (100 mL) and extracted with EtOAc (2×100 mL). The organic layer was separated, dried over anhydrous Na₂SO₄ and concentrated under vacuum. Purification by column chromatography on silica gel (using 2 to 3% MeOH in DCM as eluent) afforded the title compound (1.75 g, yield: 48%) as a yellow solid. ¹H NMR (400 MHz, CDCl₃) δ 2.31 (s, 3H), 2.39 (s, 3H), 2.83 (t, J=7.0 Hz, 2H), 3.62-3.70 (m, 2H), 4.47 (s, 2H), 6.28 (d, J=1.5 Hz, 1H), 7.23 (s, 1H), 7.29-7.36 (m, 3H), 7.61 (d, J=1.5 Hz, 1H), 7.82 (s, 1H). 2H merged in solvent peak.

### Step 6: Synthesis of 2-(1-(2-amino-4,5-dimethylphenyl)-1H-pyrazol-5-yl)ethan-1-ol N5_6

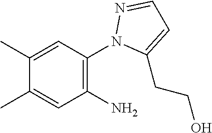 [SMILES: Cc1cc(N)c(-n2nccc2CCO)cc1C]

To a solution of 5-(2-(benzyloxy)ethyl)-1-(4,5-dimethyl-2-nitrophenyl)-1H-pyrazole (Intermediate N5_5, 1.70 g, 4.84 mmol) in dry THF (25 mL) was added 20% Pd(OH)₂/C (0.34 g, 0.48 mmol) and the reaction mixture was stirred at room temperature for 16 h under hydrogen atmosphere (P=1 atm). After completion, the reaction mixture was filtered through a pad of Celite®, washed with MeOH (25 mL) and the filtrate was concentrated under vacuum to afford the title compound (1.00 g, yield: 89%) as a yellow sticky solid. LC-MS (Method A5): [M+H]⁺ m/z: 231.9, rt: 1.56 min, purity: 92%. ¹H NMR (400 MHz, DMSO-d₆) δ 2.10 (s, 3H), 2.15 (s, 3H), 2.58 (t, J=7.3 Hz, 2H), 3.47-3.55 (m, 2H), 4.47 (s, 2H), 4.68 (t, J=5.4 Hz, 1H), 6.26 (s, 1H), 6.65 (s, 1H), 6.79 (s, 1H), 7.54 (s, 1H).

### Step 7: Synthesis of 8,9-dimethyl-4,6-dihydropyrazolo[1,5-a][1,5]benzodiazepin-5-one N5

A suspension of 2-(1-(2-amino-4,5-dimethylphenyl)-1H-pyrazol-5-yl)ethan-1-ol (0.50 g, 2.16 mmol) and K₂CO₃ (0.03 g, 0.22 mmol) in acetone (25 mL) in a steel bomb was purged with argon for 30 min. Pentamethylcyclopentadienyl rhodium dichloride dimer (0.07 g, 0.11 mmol) was added and the reaction mixture was heated at 140° C. for 16 h. After cooling to room temperature, the reaction mixture was filtered through a pad of Celite®, washed with MeOH (10 mL) and the filtrate was concentrated under vacuum. Purification by column chromatography on silica gel (using 2 to 3% MeOH in DCM as eluent) afforded the title compound (0.29 g, yield: 59%) as an off-white solid. LC-MS (Method B7): [M+H]⁺ m/z: 228.0, rt: 1.99 min, purity: 99%. ¹H NMR (400 MHz, DMSO-d₆) δ 2.24 (s, 3H), 2.27 (s, 3H), 3.58 (s, 2H), 6.37 (s, 1H), 7.00 (s, 1H), 7.57 (s, 1H), 7.72 (s, 1H), 10.11 (s, 1H).

### Intermediate N6: 13-chloro-3,10,14-triazatricyclo[9.4.0.0^{2,7}]pentadeca-1(15),2,4,6,11,13-hexaen-9-one

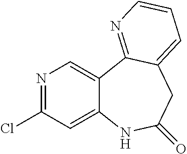 [SMILES: O=C1Cc2cccnc2-c2cnc(Cl)cc2N1]

### Step 1: Synthesis of 2-chloro-5-(4,4,5,5-tetramethyl-1,3,2-dioxaborolan-2-yl)pyridin-4-amine N6_1

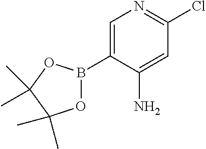 [SMILES: CC1(C)OB(c2cnc(Cl)cc2N)OC1(C)C]

A suspension of 4-amino-5-bromo-2-chloropyridine (4.40 g, 21.2 mmol), bispinacolatodiboron (5.92 g, 23.3 mmol) and KOAc (5.20 g, 53.0 mmol) in dioxane (60 mL) was purged with argon at room temperature for 20 min. PdCl₂(dppf) (0.78 g, 1.06 mmol) was added and the reaction mixture was heated at 90° C. for 16 h. After cooling to room temperature, the reaction mixture was filtered through a pad of Celite®, washed with EtOAc (100 mL) and the filtrate was concentrated under vacuum to afford the title compound (6.00 g, crude) as a brown solid. This compound was taken to the next reaction without purification. LC-MS (Method A5): [M+H]⁺ m/z: 255.0, rt: 1.96 min, purity: 37%.

### Step 2: Synthesis of 13-chloro-3,10,14-triazatricyclo[9.4.0.0^{2,7}]pentadeca-1(15),2,4,6,11,13-hexaen-9-one N6

A solution of ethyl 2-(2-chloropyridin-3-yl)acetate (2.00 g, 10.0 mmol), K₂CO₃ (3.46 g, 25.0 mmol) and 2-chloro-5-(4,4,5,5-tetramethyl-1,3,2-dioxaborolan-2-yl)pyridin-4-amine (Intermediate N6_1, 5.10 g, 20.0 mmol) in dioxane (40 mL) and H₂O (6 mL) was purged with argon for 20 min.

PdCl₂(dppf (0.37 g, 0.50 mmol) was added and the reaction mixture was heated at 100° C. for 16 h. After cooling to room temperature, the reaction mixture was concentrated under vacuum. Purification by column chromatography on silica gel (using 3% MeOH in DCM as eluent) afforded the title compound (0.55 g, yield: 22%) as an off-white solid. LC-MS (Method B7): [M+H]⁺ m/z: 246.0, rt: 1.55 min, purity: 99%. ¹H NMR (400 MHz, DMSO-d₆) δ 3.62 (s, 2H), 7.22 (s, 1H), 7.51-7.54 (m, 1H), 7.92 (d, J=8.3 Hz, 1H), 8.70 (d, J=8.3 Hz, 1H), 8.93 (s, 1H), 10.76 (s, 1H).

### Intermediate N7: 8,9-dimethyl-4,6-dihydroimidazo[2,1-d][1,5]benzodiazepin-5-one

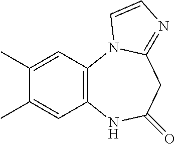 [SMILES: Cc1cc2c(cc1C)-n1ccnc1CC(=O)N2]

### Step 1: Synthesis of ethyl 3-(dimethylamino)-3-oxopropanoate N7_1

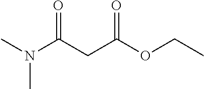

At 0° C., to a solution of dimethylamine (2 M solution in THF, 20 mL, 39.9 mmol) and TEA (9 mL, 66.4 mmol) in DCM (100 mL), was added dropwise ethyl malonyl chloride (5.00 g, 33.2 mmol) and the reaction mixture was stirred at room temperature for 12 h. After completion, the reaction mixture was treated with H₂O (200 mL) and extracted with DCM (2×100 mL). The organic layer was separated, washed with H₂O (200 mL), dried over anhydrous Na₂SO₄ and concentrated under vacuum. Purification by column chromatography on silica gel (using 5% MeOH in DCM as eluent) afforded the title compound (3.00 g, yield: 57%) as a colorless oil. LC-MS (Method A6): [M+H]⁺ m/z: 159.8, rt: 1.10 min, purity: 99%. ¹H NMR (400 MHz, DMSO-d₆) δ 1.18 (t, J=7.1 Hz, 3H), 2.82 (s, 3H), 2.94 (s, 3H), 3.48 (s, 2H), 4.08 (q, J=7.1 Hz, 2H).

### Step 2: Synthesis of 4-(dimethylamino)-7,8-dimethyl-1,3-dihydro-2H-benzo[b][1,4]diazepin-2-one N7_2

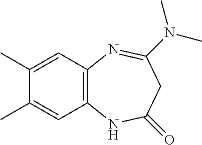

At 0° C., to a mixture of ethyl 3-(dimethylamino)-3-oxopropanoate (Intermediate N7_1, 4.21 g, 26.4 mmol) and 4,5-dimethyl-1,2-phenylenediamine (1.80 g, 13.2 mmol) was added POCl₃ (1.24 mL, 13.2 mmol) and the reaction mixture was heated at 100° C. for 4 h. After cooling to room temperature, the reaction mixture was treated with H₂O (100 mL) and heated at 70° C. for 1 h. The reaction mixture was cooled to room temperature and diluted with DCM (100 mL). The aqueous layer was separated, acidified with 2 N aqueous HCl (100 mL), treated with aqueous ammonia (20 mL) and extracted with DCM (250 mL). The organic layer was separated, dried over anhydrous Na₂SO₄ and concentrated under vacuum. The residue was dissolved in dioxane (50 mL) followed by addition of 4 M HCl in dioxane (25 mL). The reaction mixture was concentrated under vacuum and the residue was triturated with Et₂O (55 mL). The resulting solid was dissolved in H₂O (100 mL), basified with aqueous 10% Na₂CO₃ (50 mL) and extracted with DCM (250 mL). The organic layer was separated, dried over anhydrous Na₂SO₄ and concentrated under vacuum. The crude solid was purified by preparative HPLC (basic elution) to afford the title compound (0.61 g, yield: 20%) as a yellow solid. LC-MS (Method B7): [M+H]⁺ m/z: 232.0, rt: 1.98 min, purity: 95%. ¹H NMR (400 MHz, DMSO-d₆) δ 2.13 (s, 6H), 3.06 (s, 6H), 3.11 (brs, 2H), 6.75 (s, 1H), 6.81 (s, 1H), 10.0 (s, 1H).

### Step 3: Synthesis of 4-((2,2-dimethoxyethyl)amino)-7,8-dimethyl-1,3-dihydro-2H-benzo[b][1,4]diazepin-2-one N7_3

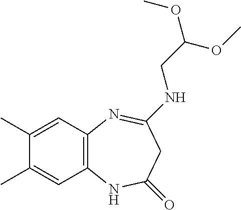

A suspension of 4-(dimethylamino)-7,8-dimethyl-1,3-dihydro-2H-benzo[b][1,4]diazepin-2-one (Intermediate N7_2, 0.40 g, 1.73 mmol), aminoacetaldehyde dimethyl acetal (0.30 mL, 3.46 mmol) and para-toluenesulfonic acid monohydrate (0.09 g, 0.52 mmol) in Dowtherm® (10 mL) was heated at 160° C. for 2 h. After cooling to room temperature, the reaction mixture was treated with H₂O (50 mL) and extracted with EtOAc (2×100 mL). The organic layer was separated, dried over anhydrous Na₂SO₄ and concentrated under vacuum. Purification by column chromatography on silica gel (using 3% MeOH in DCM as eluent) afforded the title compound (0.15 g, yield: 29%) as an off-white solid. LC-MS (Method A5): [M+H]⁺ m/z: 292.0, rt: 1.65 min, purity: 96%. ¹H NMR (400 MHz, DMSO-d₆) δ 2.13 (s, 6H), 2.95 (s, 2H), 3.38 (s, 6H), 4.52-4.55 (m, 1H), 6.74 (s, 1H), 6.81 (s, 1H), 7.32-7.36 (m, 1H), 9.89 (s, 1H). 2H proton merged into solvent peak.

### Step 4: Synthesis of 8,9-dimethyl-4,6-dihydroimidazo[2,1-d][1,5]benzodiazepin-5-one N7

A solution of 4-((2,2-dimethoxyethyl)amino)-7,8-dimethyl-1,3-dihydro-2H-benzo[b][1,4]diazepin-2-one (Intermediate N7_3, 0.30 g, 1.03 mmol) in formic acid (10 mL) was heated at 100° C. for 5 h. After cooling to room temperature, the reaction mixture was concentrated under vacuum. The residue was treated with saturated aqueous NaHCO₃ (50 mL) and extracted with EtOAc (2×100 mL). The organic layer was separated, dried over anhydrous Na₂SO₄ and concentrated under vacuum. Purification by column chromatography on silica gel (using 3% MeOH in DCM as eluent) afforded the title compound (0.15 g, yield: 62%) as an off-white solid. LC-MS (Method B7): [M+H]⁺ m/z: 228.0, rt: 1.85 min, purity: 97%. ¹H NMR (400 MHz, DMSO-d₆) δ 2.24 (s, 3H), 2.26 (s, 3H), 3.55 (s, 2H), 7.03 (s, 2H), 7.40 (s, 1H), 7.64 (s, 1H), 10.16 (s, 1H).

### Intermediate N8: 9,10-dimethyl-5,7-dihydropyrimido[5,4-d][1]benzazepin-6-one

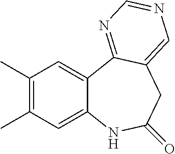 [SMILES: Cc1cc2c(cc1C)-c1ncncc1CC(=O)N2]

A mixture of ethyl 2-(4-chloropyrimidin-5-yl)acetate (0.60 g, 2.99 mmol), K₂CO₃ (1.24 g, 8.97 mmol) and 4,5-dimethyl-2-(4,4,5,5-tetramethyl-1,3,2-dioxaborolan-2-yl)aniline (Intermediate N1_3, 0.88 g, 3.59 mmol) in dioxane (12 mL) and H₂O (4 mL) was purged with argon for 30 min at room temperature. Pd(PPh₃)₄ (0.35 g, 0.30 mmol) was added and the reaction mixture was heated at 90° C. for 16 h. After cooling to room temperature, the reaction mixture was filtered through a pad of Celite® and the filtrate was partitioned between EtOAc (100 mL) and H₂O (50 mL). The organic layer was separated, washed with brine (100 mL), dried over anhydrous Na₂SO₄ and concentrated under vacuum. Purification by column chromatography on silica gel (using 2 to 3% MeOH in DCM as eluent) afforded the title compound (0.22 g, yield: 31%) as an off-white solid. LC-MS (Method B7): [M+H]⁺ m/z: 240.0, rt: 1.76 min, purity: 98%. ¹H NMR (400 MHz, DMSO-d₆) δ 2.29 (s, 3H), 2.30 (s, 3H), 3.47 (s, 2H), 7.02 (s, 1H), 7.82 (s, 1H), 8.82 (s, 1H), 9.20 (s, 1H), 10.22 (s, 1H).

### Intermediate N9: 2-chloro-9,10-dimethyl-5,7-dihydropyrido[3,2-d][1]benzazepin-6-one

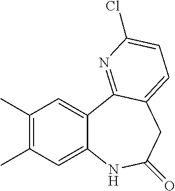 [SMILES: Cc1cc2c(cc1C)-c1nc(Cl)ccc1CC(=O)N2]

### Step 1: Synthesis of 3-(bromomethyl)-2,6-dichloropyridine N9_1

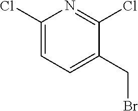

To a solution of 2,6-dichloro-3-methylpyridine (5.00 g, 30.9 mmol) in DCE (50 mL) were added NBS (6.04 g, 33.9 mmol) and AIBN (0.51 g, 3.09 mmol) and the reaction mixture was heated at 90° C. for 16 h under nitrogen atmosphere. After cooling to room temperature, the reaction mixture was treated with H₂O (50 mL) and extracted with DCM (100 mL). The organic layer was separated, washed with H₂O (100 mL), dried over anhydrous Na₂SO₄ and concentrated under vacuum. Purification by column chromatography on silica gel (using 8% EtOAc in hexanes as eluent) afforded the title compound (3.50 g, yield: 47%) as a white solid. ¹H NMR (400 MHz, DMSO-d₆) δ 4.74 (s, 2H), 7.63 (d, J=7.9 Hz, 1H), 8.15 (d, J=7.9 Hz, 1H).

### Step 2: Synthesis of 2-(2,6-dichloropyridin-3-yl)acetonitrile N9_2

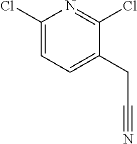 [SMILES: N#CCc1ccc(Cl)nc1Cl]

At 0° C., to a solution of 3-(bromomethyl)-2,6-dichloropyridine (Intermediate N9_1, 3.50 g, 14.5 mmol) in CH₃CN (20 mL) was added TMSCN (3.64 mL, 29.1 mmol) and the reaction mixture was stirred at the same temperature for 15 min. TBAF (1 M solution in THF, 29 mL, 29.0 mmol) was added dropwise and the reaction mixture was stirred at room temperature for 6 h. After completion, the reaction mixture was treated with H₂O (50 mL) and extracted with EtOAc (2×100 mL). The organic layer was separated, washed with H₂O (100 mL), dried over anhydrous Na₂SO₄ and concentrated under vacuum. Purification by column chromatography on silica gel (using 15% EtOAc in hexanes as eluent) afforded the title compound (1.75 g, yield: 64%) as an off-white solid. ¹H NMR (400 MHz, DMSO-de) δ 4.15 (s, 2H), 7.66 (d, J=7.9 Hz, 1H), 8.05 (d, J=7.9 Hz, 1H).

### Step 3: Synthesis of methyl 2-(2,6-dichloropyridin-3-yl)acetate N9_3

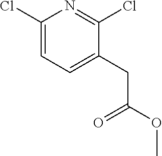

A solution of 2-(2,6-dichloropyridin-3-yl)acetonitrile (Intermediate N9_2, 1.60 g, 8.55 mmol) in a 2N solution of HCl in methanol (8 mL) was heated at 70° C. for 16 h under nitrogen atmosphere. After cooling to room temperature, the reaction mixture was concentrated under vacuum. The residue was treated with saturated aqueous NaHCO₃ solution (80 mL) and extracted with EtOAc (100 mL). The organic layer was separated, washed with brine (100 mL), dried over Na₂SO₄ and concentrated under vacuum. Purification by column chromatography on silica gel (using 35% EtOAc in hexanes as eluent) afforded the title compound (1.60 g, yield: 87%) as an off-white solid. LC-MS (Method A5): [M+H]⁺ m/z: 219.7, rt: 1.82 min, purity: 94%. ¹H NMR (400 MHz, DMSO-d₆) δ 3.65 (s, 3H), 3.88 (s, 2H), 7.59 (d, J=8.0 Hz, 1H), 7.96 (d, J=8.0 Hz, 1H).

### Step 4: Synthesis of 2-chloro-9,10-dimethyl-5,7-dihydropyrido[3,2-d][1]benzazepin-6-one N9

A mixture of methyl 2-(2,6-dichloropyridin-3-yl)acetate (Intermediate N9_3, 0.70 g, 3.18 mmol), K₂CO₃ (1.32 g, 9.54 mmol) and 4,5-dimethyl-2-(4,4,5,5-tetramethyl-1,3,2-dioxaborolan-2-yl)aniline (Intermediate N1_3, 0.79 g, 3.18 mmol) in dioxane (5 mL) and H₂O (1 mL) was purged with argon for 15 min at room temperature. Pd(PPh₃)₄ (0.18 g, 0.16 mmol) was added and the reaction mixture was heated at 90° C. for 16 h under nitrogen atmosphere. After cooling to room temperature, the reaction mixture was partitioned between EtOAc (100 mL) and H₂O (50 mL). The organic layer was separated, washed with brine (100 mL), dried over anhydrous Na₂SO₄ and concentrated under vacuum. Purification by column chromatography on silica gel (using 5% MeOH in DCM as eluent) afforded the title compound (0.065 g, yield: 7%) as an off-white solid. LC-MS (Method A5): [M+H]⁺ m/z: 273.0, rt: 2.50 min, purity: 98%. ¹H NMR (400 MHz, DMSO-d₆) δ 2.28 (s, 3H), 2.29 (s, 3H), 3.42 (s, 2H), 6.98 (s, 1H), 7.52 (d, J=8.0 Hz, 1H), 7.67 (s, 1H), 7.90 (d, J=8.0 Hz, 1H), 10.11 (s, 1H).

### Intermediate N10: 5-chloro-4,8,14-triazatricyclo[9.4.0.0^{2,7}]pentadeca-1(15),2,4,6,11,13-hexaen-9-one

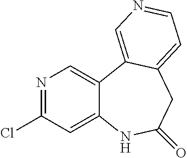 [SMILES: O=C1Cc2ccncc2-c2cnc(Cl)cc2N1]

The title compound was prepared according to the procedure of intermediate N12, starting from methyl 2-(3-bromopyridin-4-yl)acetate in step 2. Purification by column chromatography on silica gel (using 3% MeOH in DCM as eluent) afforded the title product (0.40 g, yield: 25%) as an off-white solid. LC-MS (Method B7): [M+H]⁺ m/z: 246.0, rt: 1.22 min, purity: 94%. ¹H NMR (400 MHz, DMSO-d₆) δ 3.64 (s, 2H), 7.25 (s, 1H), 7.52 (d, J=5.2 Hz, 1H) 8.66 (d, J=5.2 Hz, 1H) 8.78 (s, 1H) 8.88 (s, 1H) 10.8 (s, 1H).

### Intermediate N11: 3-chloro-5,7-dihydropyrido[4,3-d][3]benzazepin-6-one

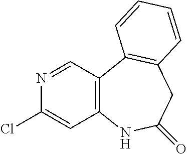 [SMILES: O=C1Cc2ccccc2-c2cnc(Cl)cc2N1]

The title compound was prepared according to the procedure of intermediate N12, using 2-(2-(4,4,5,5-tetramethyl-1,3,2-dioxaborolan-2-yl)phenyl)acetate in step 2. Purification by trituration in DCM afforded the title product (30 mg, yield: 52%) as a beige solid. It was taken to the next step without further purification. LC-MS (Method B1): [M−H]⁻ m/z: 243.0, rt: 2.00 min, purity: 87%.

### Intermediate N12: 5-chloro-4,8,12-triazatricyclo[9.4.0.0^{2,7}]pentadeca-1(11),2(7),3,5,12,14-hexan-9-one

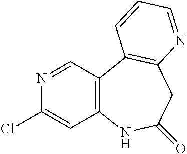 [SMILES: O=C1Cc2ncccc2-c2cnc(Cl)cc2N1]

### Step 1: Synthesis of 2-[3-(4,6-dichloro-3-pyridyl)-2-pyridyl]acetonitrile N12_1

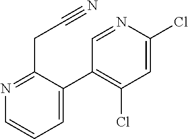 [SMILES: N#CCc1ncccc1-c1cnc(Cl)cc1Cl]

Under inert atmosphere, to a solution of 2-(3-bromopyridin-2-yl)acetonitrile (302 mg, 1.50 mmol) and 4,6-dichloropyridine-3-boronic acid (587 mg, 3.00 mmol) in dioxane (12 mL) were added bis(di-tert-butyl(4-dimethylaminophenyl)phosphine)dichloropalladium(II) (112 m, 0.15 mmol) followed by a 2M solution of Na₂CO₃ in water (3 mL). The reaction mixture was sonicated a few seconds and then heated at 80° C. for 3 h. After cooling to room temperature, water was added, and the reaction mixture was extracted with EtOAc (3×). The combined organic layers were washed with brine, dried over MgSO₄, filtered off and concentrated under vacuum. The crude solid was purified by flash column chromatography on silica gel (using a gradient of heptane/EtOAc 100:0 to 50:50 as eluent) to afford the title compound as an orange oil (200 mg, yield: 47%). LC-MS (Method B1) m/z: [M+H]⁺: 265.9, rt: 1.15 min, purity: 93%. ¹H NMR (400 MHz, DMSO-d₆) δ 8.71 (d, J=4.9 Hz, 1H), 8.46 (s, 1H), 8.04 (s, 1H), 7.81 (d, J=7.7 Hz, 1H), 7.55 (dd, J=7.7, 4.9 Hz, 1H), 4.13-3.90 (n, 2H).

### Step 2: Synthesis of 2-[3-(4,6-dichloro-3-pyridyl)-2-pyridyl]acetamide N12_2

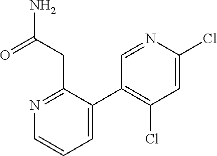 [SMILES: NC(=O)Cc1ncccc1-c1cnc(Cl)cc1Cl]

A suspension of 2-[3-(4,6-dichloro-3-pyridyl)-2-pyridyl]acetonitrile (Intermediate N12_1, 200 mg, 0.70 mmol) in concentrated sulfuric acid (1 mL) was heated at 60° C. for 2 h. After cooling to 0° C., the reaction mixture was slowly neutralized by addition of saturated aqueous NaHCO₃ and extracted with EtOAc (3×20 mL). The combined organic layers were dried over MgSO₄, filtered off and concentrated under vacuum to afford the title compound as a yellow solid (175 mg, yield: 80%). LC-MS (Method B1) m/z: [M+H]⁺: 282.0, rt: 0.94 min, purity: 91%. ¹H NMR (400 MHz, DMSO-d₆) δ 8.61 (d, J=4.9 Hz, 1H), 8.39 (s, 1H), 7.98 (s, 1H), 7.70 (d, J=7.7 Hz, 1H), 7.42 (dd, J=7.7, 4.9 Hz, 1H), 7.32 (s, 1H), 6.85 (s, 1H), 3.55 (d, J=15.2 Hz, 1H), 3.36 (d, J=15.2 Hz, 1H).

### Step 3: Synthesis of 5-chloro-4,8,12-triazatricyclo[9.4.0.0^{2,7}]pentadeca-1(11),2(7),3,5,12,14-hexan-9-one N12

At 0° C., to a solution of 2-[3-(4,6-dichloro-3-pyridyl)-2-pyridyl]acetamide (Intermediate N12_2, 876 mg, 2.98 mmol) in dry DMF (15 mL) was slowly added sodium hydride (60% suspension in oil, 240 mg, 6.0 mmol) and the resulting mixture was stirred at room temperature for 20 h. The reaction mixture was poured into cold water and extracted with EtOAc (3×30 mL). The combined organic layers were washed with brine, dried over MgSO₄, filtered off and concentrated under vacuum to give a brown solid. Trituration in DCM afforded the title compound as a light brown solid (402 mg, yield: 55%). LC-MS (Method B1) m/z: [M+H]⁺: 246.0, rt: 0.82 min, purity>99%. ¹H NMR (400 MHz, DMSO-d₆) δ 10.82 (s, 1H), 8.72 (s, 1H), 8.64 (dd, J=4.8, 1.6 Hz, 1H), 8.16 (dd, J=7.8, 1.6 Hz, 1H), 7.54 (dd, J=7.8, 4.8 Hz, 1H), 7.26 (s, 1H), 3.76 (s, 2H).

### Intermediate N13: 12-chloro-5-thia-9,13-diazatricyclo[8.4.0.0^{2,6}]tetradeca-1(10),2(6),3,11,13-pentaen-8-one

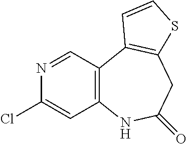 [SMILES: O=C1Cc2sccc2-c2cnc(Cl)cc2N1]

### Step 1: Synthesis of ethyl 2-(3-bromothiophen-2-yl)acetate N13_1

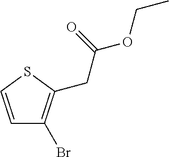

To a solution of 3-bromothiophene-2-carbaldehyde (6.00 g, 31.4 mmol) in dry THF (150 mL) were added methylsulfanyl(methylsulfinyl)methane (10 mL, 97.4 mmol) and a 40% solution of benzyltrimethylammonium hydroxide in methanol (8.5 mL, 18.8 mmol) and the reaction mixture was heated at 60° C. for 4 h under nitrogen. After cooling to room temperature, the reaction mixture was poured into a 0.5N HCl solution and extracted with EtOAc (2×100 mL). The combined organic layers were washed with brine (100 mL), dried over anhydrous Na₂SO₄ and concentrated under vacuum. The residue was dissolved in a 0.5 N solution of HCl in EtOH (50 mL) and the reaction mixture was heated at reflux for 2 h. The solvent was removed under vacuum and the crude mixture was diluted with water (100 mL) and extracted with EtOAc (2×100 mL). The combined organic layers were washed with water (100 mL), dried over anhydrous Na₂SO₄ and concentrated under vacuum. The residue was purified by column chromatography on silica gel (using a gradient of 10 to 15% EtOAc in hexanes as eluent) to afford the title product (2.50 g, yield: 29%) as a pale-yellow oil. ¹H NMR (400 MHz, CDCl₃) δ 7.24 (d, J=5.4 Hz, 1H), 6.96 (d, J=5.4 Hz, 1H), 4.21 (q, J=7.3 Hz, 2H), 3.83 (s, 2H), 1.30 (t, J=7.3 Hz, 3H).

### Step 2: Synthesis of ethyl 2-(3-(4-amino-6-chloropyridin-3-yl)thiophen-2-yl)acetate N13_2

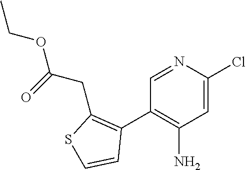 [SMILES: CCOC(=O)Cc1sccc1-c1cnc(Cl)cc1N]

To a solution of ethyl 2-(3-bromothiophen-2-yl)acetate (Intermediate N13_1, 0.35 g, 1.40 mmol) in dioxane (5 mL) and water (1 mL) were added K₂CO₃ (0.58 g, 4.21 mmol) and 2-chloro-5-(4,4,5,5-tetramethyl-1,3,2-dioxaborolan-2-yl)pyridin-4-amine (Intermediate N6_1, 1.07 g, 4.21 mmol) and the reaction mixture was purged with argon for 30 min. PdCl₂(dppf (0.05 g, 0.07 mmol) was added and the reaction mixture was heated at 80° C. for 16 h. After cooling to room temperature, the reaction mixture was concentrated under vacuum and the residue was directly purified by column chromatography on silica gel (using a gradient of 3 to 5% MeOH in DCM as eluent) to afford ethyl 2-(3-(4-amino-6-chloropyridin-3-yl)thiophen-2-yl)acetate (0.07 g, yield: 17%) as an off-white solid and 3-chloro-5,7-dihydro-6H-pyrido[4,3-b]thieno[3,2-d]azepin-6-one (0.05 g, yield: 14%) as an off-white solid. LC-MS (method A5) m/z: [M+H]⁺: 296.7, rt: 1.80 min, purity: 73%.

### Step 3: Synthesis of 12-chloro-5-thia-9,13-diazatricyclo[8.4.0.02 ⁶]tetradeca-1(10),2(6),3,11,13-pentaen-8-one N13

At 0° C., to a solution of ethyl 2-(3-(4-amino-6-chloropyridin-3-yl)thiophen-2-yl)acetate (Intermediate N13_2, 0.07 g, 0.24 mmol) in dry DMF (2 mL) was added NaH (60% dispersion in oil, 0.01 g, 0.35 mmol) and the reaction mixture was stirred at room temperature for 16 h. The reaction mixture was diluted with water (25 mL) and extracted with EtOAc (2×25 mL). The combined organic layers were washed with brine (25 mL), dried over anhydrous Na₂SO₄ and concentrated under vacuum. The residue was purified by column chromatography on silica gel (using a gradient of 3 to 5% MeOH in DCM as eluent) to afford the title product (0.03 g, yield: 51%) as an off-white solid. MS (method A5) m/z: [M+H]⁺: 251.0, rt: 1.94 min, purity: 98%. ¹H NMR (400 MHz, DMSO-d₆) δ 10.67 (bs, 1H), 8.69 (s, 1H), 7.58 (d, J=5.4 Hz, 1H), 7.50 (d, J=5.4 Hz, 1H), 7.20 (s, 1H), 3.69 (s, 2H).

### Intermediate N14: 1,3-dimethyl-5,7-dihydropyrido[4,3-d][3]benzazepin-6-one

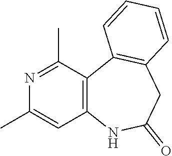 [SMILES: Cc1cc2c(c(C)n1)-c1ccccc1CC(=O)N2]

### Step 1: Synthesis of methyl 2-[2-(4-amino-2,6-dimethyl-3-pyridyl)phenyl]acetate N14_1

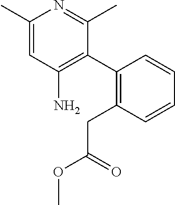 [SMILES: COC(=O)Cc1ccccc1-c1c(N)cc(C)nc1C]

Under inert atmosphere, a mixture of 4-amino-3-bromo-2,6-dimethylpyridine (50 mg, 0.24 mmol), cesium carbonate (59 mg, 0.18 mmol), methyl 2-(2-(4,4,5,5-tetramethyl-1,3,2-dioxaborolan-2-yl)phenyl)acetate (31 mg, 0.11 mmol) and Pd(PPh₃)₄ (11 mg, 0.01 mmol) in 1,2-dimethoxyethane (1 mL) was heated at 120° C. for 4 h. After cooling to room temperature, the reaction mixture was filtered through a pad of Celite® with EtOAc and the filtrate was concentrated under vacuum to give the title compound (62 mg, yield: 94%) as a yellow solid which was taken crude to the next step.

LC-MS (Method B1): [M+H]⁺ m/z: 271.0, rt: 2.12 min.

### Step 2: Synthesis of 1,3-dimethyl-5,7-dihydropyrido[4,3-d][3]benzazepin-6-one N14

At room temperature, to a solution of methyl 2-[2-(4-amino-2,6-dimethyl-3-pyridyl)phenyl]acetate (Intermediate N14_1, 62 mg, 0.23 mmol) in dry toluene (2.3 mL) was added dropwise a 1M solution of lithium bis(trimethylsilyl)amide in THF (690 μL, 0.69 mmol) and the reaction mixture was stirred at room temperature for 2 h. The reaction mixture was treated with saturated aqueous NH₄Cl solution and extracted twice with EtOAc. The organic layer was separated, washed with brine, dried over Na₂SO₄ and concentrated under vacuum. Purification by preparative TLC on silica gel (using 10% MeOH in DCM as eluent) afforded the title compound (8 mg, yield: 15%) as an off-white solid. LC-MS (Method B1): [M+H]⁺ m/z: 239.0, rt: 1.81 min, purity>90%.

### Intermediate N15: 12-chloro-5,6,9,13-tetrazatricyclo[8.4.0.0^{2,6}]tetradeca-1(10),2,4,11,13-pentaen-8-one

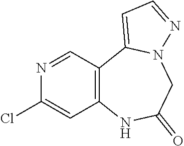 [SMILES: O=C1Cn2nccc2-c2cnc(Cl)cc2N1]

### Step 1: Synthesis of 2-chloro-5-(1-(tetrahydro-2H-pyran-2-yl)-1H-pyrazol-5-yl)pyridin-4-amine N15_1

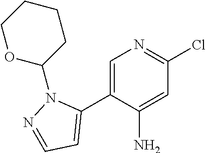 [SMILES: Nc1cc(Cl)ncc1-c1ccnn1C1CCCCO1]

To a solution of 4-amino-5-bromo-2-chloropyridine (1.20 g, 5.78 mmol) in dioxane (20 mL) were added K₂CO₃ (2.00 g, 14.5 mmol) and 1-tetrahydropyran-2-yl-5-(4,4,5,5-tetramethyl-1,3,2-dioxaborolan-2-yl)pyrazole (3.22 g, 11.6 mmol) and the reaction mixture was purged with argon for 30 min. SPhos Pd G₂ (0.23 g, 0.31 mmol) and Pd₂(dba)₃ (0.27 g, 0.29 mmol) were added at room temperature and the reaction mixture was heated at 80° C. for 16 h. After completion, the reaction mixture was concentrated under vacuum and the residue was purified by column chromatography on silica gel (using 3 to 5% MeOH in DCM as eluent) to afford the title product (1.00 g, yield: 62%) as a yellow sticky solid. LC-MS (Method A5): [M+H]⁺ m/z: 279.0, rt: 1.69 min, purity: 86%. ¹H NMR (400 MHz, DMSO-d₆) δ 7.93 (s, 1H), 7.64 (d, J=1.0 Hz, 1H), 6.71 (s, 1H), 6.40 (d, J=1.0 Hz, 1H), 6.19 (bs, 2H), 5.01 (dd, J=9.8, 2.0 Hz, 1H), 3.39-3.49 (m, 2H), 2.28-2.38 (m, 2H), 1.80-1.98 (m, 2H), 1.46-1.60 (m, 2H).

### Step 2: Synthesis of 2-chloro-N-(2-chloro-5-(1H-pyrazol-5-yl)pyridin-4-yl)acetamide N15_2

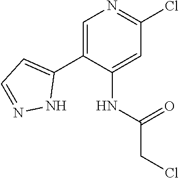 [SMILES: O=C(CCl)Nc1cc(Cl)ncc1-c1ccn[nH]1]

At 0° C., to a solution of 2-chloro-5-(1-(tetrahydro-2H-pyran-2-yl)-1H-pyrazol-5-yl)pyridin-4-amine (Intermediate N15_1, 0.50 g, 1.79 mmol) in THF (10 mL) was added NaH (60% dispersion in oil, 0.11 g, 2.69 mmol) and the reaction mixture was stirred at room temperature for 10 min. 2-Chloroacetyl chloride (0.3 mL, 3.59 mmol) was added at 0° C. and the reaction mixture was stirred at room temperature for 16 h. After completion, the reaction mixture was diluted with H₂O (25 mL), neutralized with aqueous saturated NaHCO₃ solution (25 mL) and extracted with EtOAc (2×50 mL). The organic layer was separated, dried over anhydrous Na₂SO₄, filtered off and concentrated under vacuum. Purification by column chromatography on silica gel (using 3 to 5% MeOH in DCM as eluent) afforded the title product (0.20 g, yield: 41%) as an off-white solid. LC-MS (Method A5): [M+H]⁺ m/z: 270.9, rt: 1.84 min, purity: 82%. ¹H NMR (400 MHz, DMSO-d₆) δ 13.56 (bs, 1H), 12.57 (bs, 1H), 8.88 (s, 1H), 8.54 (s, 1H), 8.00 (d, J=1.9 Hz, 1H), 7.07 (d, J=1.9 Hz, 1H), 4.53 (s, 2H).

### Step 3: Synthesis of 12-chloro-5,6,9,13-tetrazatricyclo[8.4.0.0^{2,6}]tetradeca-1(10),2,4,11,13-pentaen-8-one N15

To a solution of 2-chloro-N-(2-chloro-5-(1H-pyrazol-5-yl)pyridin-4-yl)acetamide (Intermediate N15_2, 0.40 g, 1.48 mmol) in dry DMF (10 mL) was added NaH (60% dispersion in oil, 0.09 g, 2.21 mmol) and the reaction mixture was stirred at room temperature for 16 h. After completion, the reaction mixture was diluted with H₂O (25 mL) and extracted with EtOAc (2×50 mL). The organic layer was separated, dried over anhydrous Na₂SO₄ and concentrated under vacuum. Purification by column chromatography on silica gel (using 3 to 5% MeOH in DCM as eluent) afforded the title product (0.165 g, yield: 48%) as an off-white solid. LC-MS (Method B7): [M+H]⁺ m/z: 235.0, rt: 1.84 min, purity: 99%. ¹H NMR (400 MHz, DMSO-d₆) δ 11.07 (bs, 1H), 8.70 (d, J=1.9 Hz, 1H), 7.63 (s, 1H), 7.26 (d, J=1.9 Hz, 1H), 6.86 (s, 1H), 4.98 (s, 2H).

### Intermediate N16: 15-fluoro-13-methyl-3,10,14-triazatricyclo[9.4.0.0^{2,7}]pentadeca-1(11),2(7),3,5,12,14-hexaen-9-one

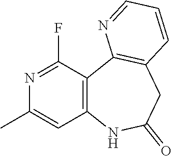 [SMILES: Cc1cc2c(c(F)n1)-c1ncccc1CC(=O)N2]

### Step 1: Synthesis of 2-fluoro-6-methyl-3-(4,4,5,5-tetramethyl-1,3,2-dioxaborolan-2-yl)pyridin-4-amine N16_1

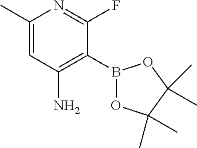

In a nitrogen filled glovebox, 2-fluoro-6-methyl-pyridin-4-amine (Intermediate NN445, 138 mg, 1.04 mmol) was dissolved in dry THF (1 mL) in a 6 mL pressure tube containing a magnetic stir bar. Pinacolborane (211 μL, 1.45 mmol) was added and the reaction mixture was stirred at room temperature for 1 h. (1,5-Cyclooctadiene)(methoxy)iridium(I) dimer (10 mg, 0.015 mmol), 4,4′-di-tert-butyl-2,2′-dipyridyl (9 mg, 0.033 mmol) and bis(pinacolato)diboron (158 mg, 0.62 mmol) were added, the tube was sealed under nitrogen atmosphere and the reaction mixture was heated at 80° C. for 16 h. After cooling to room temperature, methanol (3 mL) was added and the reaction mixture was stirred for 10 min until the gas evolution has ceased, before being concentrated under vacuum. The crude brown oil was purified by column chromatography on silica gel (using a gradient DCM/EtOAc from 100/0 to 50/50 as eluent) and by trituration in hexane to afford the title compound as light pink solid (200 mg, yield: 72%). LC-MS (Method B1) m/z: [M+H]P: 253.0, rt: 1.13 min, purity: 95%. ¹H NMR (400 MHz, DMSO-d₆) δ 6.58 (s, 2H), 6.29 (d, J=1.9 Hz, 1H), 2.16 (s, 3H), 1.28 (s, 12H).

### Step 2: Synthesis of 15-fluoro-13-methyl-3,10,14-triazatricyclo[9.4.0.0^{2,7}]pentadeca-1(11),2(7),3,5,12,14-hexaen-9-one N16

To a solution of 2-fluoro-6-methyl-3-(4,4,5,5-tetramethyl-1,3,2-dioxaborolan-2-yl)pyridin-4-amine (Intermediate N16_1, 237 mg, 0.94 mmol) and methyl 2-(2-chloro-3-pyridyl)acetate (175 mg, 0.94 mmol) in dioxane (4.7 mL) were added K₂CO₃ (394 mg, 2.82 mmol) and water (0.3 mL). The resulting mixture was degassed and filled with N₂ before the addition of 1,1′-bis(diphenylphosphino)ferrocene-palladium(II)dichloride dichloromethane complex (39 mg, 0.047 mmol). The reaction mixture was heated at 100° C. for 16 h. After cooling to room temperature, the reaction mixture was treated with water and extracted with EtOAc (3×). The combined organic layers were washed with brine, dried over Na₂SO₄, filtered off and concentrated under vacuum. The crude brown oil was triturated in Et₂O to afford the title compound as a brown solid (70 mg, yield: 28%). LC-MS (Method A1) m/z: [M+H]⁺: 244.0, rt: 0.67 min, purity: 92%. ¹H NMR (400 MHz, DMSO-d₆) δ 10.70 (s, 1H), 8.64 (d, J=4.7 Hz, 1H), 7.89 (d, J=7.8 Hz, 1H), 7.47 (dd, J=7.8, 4.7 Hz, 1H), 6.96 (s, 1H), 3.61 (d, J=12.8 Hz, 1H), 3.54 (d, J=12.8 Hz, 1H).

### Intermediate N17: 9-chloro-3-methyl-5,7-dihydropyrido[4,3-d][3]benzazepin-6-one

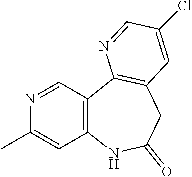 [SMILES: Cc1cc2c(cn1)-c1ncc(Cl)cc1CC(=O)N2]

A suspension of 4-amino-5-bromo-2-methylpyridine (200 mg, 1.05 mmol), methyl 2-(5-chloro-2-(4,4,5,5-tetramethyl-1,3,2-dioxaborolan-2-yl)phenyl)acetate (514 mg, 1.56 mmol) and cesium carbonate (684 mg, 2.1 mmol) in 1,2-dimethoxyethane (10 mL) was purged with argon for 20 min. 1,1′-bis(diphenylphosphino)ferrocene-palladium(II)dichloride dichloromethane complex (86 mg, 0.10 mmol) was added and the reaction mixture was heated at 140° C. for 6 h. After cooling to room temperature, water was added and the reaction mixture was extracted 3 times with EtOAc. The combined organic layers were washed with water, brine, dried over Na₂SO₄, filtered off and concentrated under vacuum. The crude brown oil was purified by trituration in diethyl ether to afford the title compound as a beige solid (84 mg, yield: 31%). LC-MS (Method B1) m/z: [M+H]⁺: 259.0; rt: 1.98 min; purity: 85%.

### Intermediate N18: 12-methyl-5,6,9,13-tetrazatricyclo[8.4.0.0^{2,6}]tetradeca-1(10),2,4,11,13-pentaen-8-one

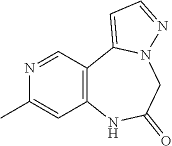 [SMILES: Cc1cc2c(cn1)-c1ccnn1CC(=O)N2]

Under inert atmosphere, to a suspension of intermediate N15 (50 mg, 0.21 mmol), 1,1′-bis(diphenylphosphino)ferrocene-palladium(II)dichloride dichloromethane complex (17 mg, 0.02 mmol) and cuprous iodide (4 mg, 0.02 mmol) in dry THF (2 mL), was added dropwise a 2M solution of methyl zinc chloride in THF (0.42 mL, 0.84 mmol) and the reaction mixture was heated at 70° C. for 16 h. After cooling to room temperature, the reaction mixture was filtered through a pad of Celite® with DMF and the filtrate was purified by reverse phase chromatography to afford the title compound (23 mg, yield: 44%) as a white solid. LC-MS (Method B1): [M+H]⁺ m/z: 215.1, rt: 0.73 min, purity>99%.

### Intermediate N19: 1-chloro-3-methyl-5,7-dihydropyrido[4,3-d][3]benzazepin-6-one

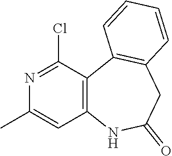 [SMILES: Cc1cc2c(c(Cl)n1)-c1ccccc1CC(=O)N2]

### Step 1: Synthesis of 2-chloro-3-iodo-6-methyl-pyridin-4-amine N19_1

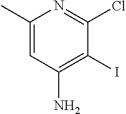

To a solution of 2-chloro-6-methylpyridin-4-amine (2.0 g, 13.3 mmol) in dry acetonitrile (67 mL) was added N-iodosuccinimide (3.15 g, 14.0 mmol) and the reaction mixture was heated at 80° C. for 22 h. The reaction mixture was concentrated under vacuum and the residue was partitioned between EtOAc and saturated aqueous Na₂S₂O₃. The phases were separated, and the aqueous layer was extracted twice with EtOAc. The combined organic layers were washed with brine, dried over Na₂SO₄, filtered off and concentrated under vacuum. The crude yellow solid was purified by column chromatography on silica gel (using a gradient of heptane/EtOAc from 100/0 to 50/50 as eluent) to afford the title product as a grey solid (1.69 g, yield: 47%). LC-MS (Method A1) m/z: [M+H]⁺: 269.0, rt: 0.72 min, purity>99%. ¹H NMR (400 MHz, DMSO-d₆) δ 6.41 (s, 2H), 6.39 (s, 1H), 2.19 (s, 3H).

### Step 2: Synthesis of methyl 2-[2-(4-amino-2-chloro-6-methyl-3-pyridyl)phenyl]acetate N19_2

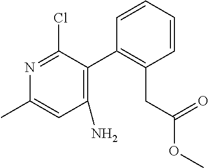

Under inert atmosphere, to a solution of 2-chloro-3-iodo-6-methyl-pyridin-4-amine (Intermediate N19_1, 1.35 g, 5.03 mmol) in 1,4-dioxane (40 mL) were added methyl 2-(2-(4,4,5,5-tetramethyl-1,3,2-dioxaborolan-2-yl)phenyl)acetate (1.9 g, 6.50 mmol), bis(di-tert-butyl(4-dimethylaminophenyl)phosphine)dichloropalladium(II) (356 mg, 0.50 mmol), K₂CO₃ (2.11 g, 15.1 mmol) and water (10 mL). The reaction mixture was sonicated a few seconds and heated at 100° C. for 3 h. After cooling to room temperature, water was added, and the mixture was extracted with EtOAc (3×). The combined organic layers were washed with brine, dried over MgSO₄, filtered off and concentrated under vacuum to afford the title compound as a brown oil (2.46 g, yield: 79%) which was taken crude to the next step. LC-MS (Method B1) m/z: [M+H]⁺: 290.9, rt: 1.09 min, purity: 74%. ¹H NMR (400 MHz, DMSO-d₆) δ 7.47-7.33 (m, 4H), 6.49 (s, 1H), 5.46 (s, 2H), 3.47 (s, 3H), 3.44 (s, 2H), 2.26 (s, 3H).

### Step 3: Synthesis of 1-chloro-3-methyl-5,7-dihydropyrido[4,3-d][3]benzazepin-6-one N19

The title product was prepared following the same procedure as for intermediate N14, step 2, starting from methyl 2-[2-(4-amino-2-chloro-6-methyl-3-pyridyl)phenyl]acetate (Intermediate N19_2, 185 mg, 0.64 mmol). Purification by trituration in Et₂O afforded the title compound (70 mg, yield: 41%) as a beige solid. LC-MS (Method B1): [M+H]⁺ m/z: 258.9, rt: 1.09 min, purity>99%. ¹H NMR (400 MHz, DMSO-d₆) δ 10.47 (s, 1H), 7.85-7.61 (m, 2H), 7.53-7.26 (m, 3H), 3.63-3.39 (m, 2H), 2.46 (s, 3H).

### Intermediate N20: 3,5-dimethyl-4,8,12-triazatricyclo[9.4.0.0^{2,7}]pentadeca-1(11),2(7),3,5,12,14-hexaen-9-one

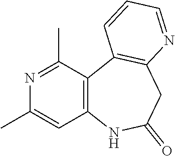 [SMILES: Cc1cc2c(c(C)n1)-c1cccnc1CC(=O)N2]

A suspension of 3-bromo-2,6-dimethylpyridin-4-amine (200 mg, 0.97 mmol), methyl 2-[3-(4,4,5,5-tetramethyl-1,3,2-dioxaborolan-2-yl)-2-pyridyl]acetate (Intermediate N25_2, 341 mg, 1.69 mmol) and potassium phosphate tribasic (640 mg, 2.92 mmol) in toluene (4.9 mL) was purged with argon for 20 min. tris(dibenzylideneacetone)dipalladium(0) (92 mg, 0.10 mmol) and 2-dicyclohexylphosphino-2,6-dimethoxybiphenyl (82 mg, 0.96 mmol) were added and the reaction mixture was heated at 120° C. for 20 h. After cooling to room temperature, water was added, and the reaction mixture was extracted 3 times with EtOAc. The combined organic layers were washed with water, brine, dried over Na₂SO₄, filtered off and concentrated under vacuum to give an orange solid. Purification by trituration in diethyl ether afforded the tittle compound as a beige solid (114 mg, yield: 49%). LC-MS (Method B1) m/z: [M+H]⁺: 240.0; rt: 0.74 min; purity: 98%.

### Intermediate N21: 9-chloro-1,3-dimethyl-5,7-dihydropyrido[4,3-d][3]benzazepin-6-one

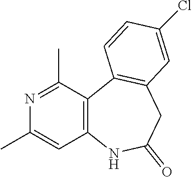 [SMILES: Cc1cc2c(c(C)n1)-c1ccc(Cl)cc1CC(=O)N2]

### Step 1: Synthesis of methyl 2-[2-(4-amino-2,6-dimethyl-3-pyridyl)-5-chloro-phenyl]acetate N21_1

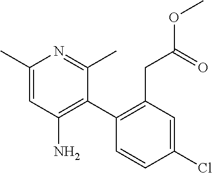

Under inert atmosphere, a mixture of 4-amino-3-bromo-2,6-dimethyl-pyridine (100 mg, 0.49 mmol), K₂CO₃ (204 mg, 1.46 mmol), methyl 2-(5-chloro-2-(4,4,5,5-tetramethyl-1,3,2-dioxaborolan-2-yl)phenyl)acetate (191 mg, 0.58 mmol) and bis(di-tert-butyl(4-dimethylaminophenyl)phosphine)dichloropalladium(II) (35 mg, 0.05 mmol) in dioxane (3.9 mL) and water (1.0 mL) was heated at 100° C. for 3.5 h. After cooling to room temperature, the reaction mixture was partitioned between EtOAc and water and extracted twice with EtOAc. The combined organic extracts were dried with brine and over MgSO₄, filtered off and concentrated under vacuum to give the title compound (155 mg, quantitative yield) as a yellow oil which was taken crude to the next step. LC-MS (Method B1): [M+H]⁺ m/z: 305.0, rt: 1.22 min.

### Step 2: Synthesis of 9-chloro-1,3-dimethyl-5,7-dihydropyrido[4,3-d][3]benzazepin-6-one N21

The title product was prepared following the same procedure as for intermediate N14, step 2, starting from methyl 2-[2-(4-amino-2,6-dimethyl-3-pyridyl)-5-chloro-phenyl]acetate (Intermediate N21_2, 149 mg, 0.49 mmol). Purification by trituration in Et₂O afforded the title compound (107 mg, yield: 80%) as a beige solid. LC-MS (Method B1): [M+H]⁺ m/z: 273.0, rt: 1.12 min, purity: 95%.

### Intermediate N22: 3-fluoro-10-hydroxy-5-methyl-4,8,12-triazatricyclo[9.4.0.0^{2,7}]pentadeca-1(11),2(7),3,5,12,14-hexaen-9-one

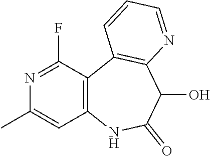 [SMILES: Cc1cc2c(c(F)n1)-c1cccnc1C(O)C(=O)N2]

### Step 1: Synthesis of ethyl 2-acetoxy-2-(3-bromo-2-pyridyl)acetate N22_1

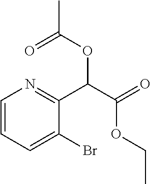 [SMILES: CCOC(=O)C(OC(C)=O)c1ncccc1Br]

To a solution of ethyl 2-(3-bromo-2-pyridyl)acetate (Intermediate N3_1, 3.9 g, 15.6 mmol) in DCM (60 mL) was added iodobenzene diacetate (5.6 g, 17.0 mmol) and the reaction mixture was stirred at room temperature for 24 h. The reaction mixture was treated with 50 mL of water, the DCM phase was separated with a Phase Separator Syringe and the organic layer was concentrated under vacuum. Purification by column chromatography on silica gel (using 20% EtOAc in hexanes as eluent) afforded the title compound as a light-yellow solid (3.5 g, yield: 75%). LC-MS (Method B1) m/z: [M+H]⁺: 302.0; rt: 1.22 min; purity.

### Step 2: Synthesis of ethyl 2-acetoxy-2-[3-(4-amino-2-fluoro-6-methyl-3-pyridyl)-2-pyridyl]acetate N22_2

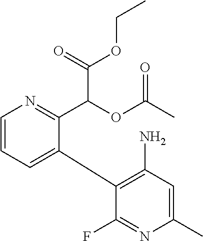 [SMILES: CCOC(=O)C(OC(C)=O)c1ncccc1-c1c(N)cc(C)nc1F]

To a solution of 2-fluoro-6-methyl-3-(4,4,5,5-tetramethyl-1,3,2-dioxaborolan-2-yl)pyridin-4-amine (Intermediate N16_1, 200 mg, 0.71 mmol) and ethyl 2-acetoxy-2-(3-bromo-2-pyridyl)acetate (Intermediate N22_1, 216 mg, 0.71 mmol) in dry toluene (4 mL) was added potassium phosphate tribasic (312 mg, 1.42 mmol). Argon was passed through the reaction mixture before addition of tris(dibenzylideneacetone)dipalladium(0) (65 mg, 0.07 mmol) and 2-dicyclohexylphosphino-2,6-dimethoxybiphenyl (30 mg, 0.07 mmol). The reaction mixture was then stirred at 100° C. for 5 h. After cooling to room temperature, the reaction mixture was diluted with EtOAc, filtered over a PTFE filter and the filtrate was concentrated to dryness affording the crude title compound as a brown foam which was taken to the next step without purification. LC-MS (Method A1) m/z: [M+H]⁺: 348; rt: 0.94 and 1.08 min as a mixture of diastereisomers; purity: 32%.

### Step 3: Synthesis of 3-fluoro-10-hydroxy-5-methyl-4,8,12-triazatricyclo[9.4.0.0^{2,7}]pentadeca-1(11),2(7),3,5,12,14-hexaen-9-one N22

At 0° C., to a solution of ethyl 2-acetoxy-2-[3-(4-amino-2-fluoro-6-methyl-3-pyridyl)-2-pyridyl]acetate (Intermediate N22_2, 248 mg, 0.71 mmol) in dry toluene (4 mL), was added a 1.5 M solution of lithium bis(trimethylsilyl)amide in THF (1.4 mL, 2.10 mmol) and the reaction mixture was stirred at room temperature for 1 h. The reaction mixture was neutralized by addition of saturated aqueous NH₄Cl and extracted with EtOAc (3×). The combined organic layers were washed with brine, dried over Na₂SO₄, filtered off and concentrated under vacuum. The residue was purified by trituration in Et₂O to afford the title compound as a beige solid (135 mg, yield: 65%). LC-MS (Method A1) m/z: [M+H]⁺: 260.0; rt: 0.69 min; purity: 95%. ¹H NMR (400 MHz, DMSO-d₆) of the major diastereoisomer (7:3) δ 10.92 (bs, 1H), 8.70 (d, J=4.7 Hz, 1H), 8.14 (ddd, J=8.0, 4.7, 1.6 Hz, 1H), 7.54 (dd, J=8.0, 4.7 Hz, 1H), 7.01 (s, 1H), 5.59 (d, J=8.2 Hz, 1H), 5.01 (d, J=8.2 Hz, 1H), 2.46 (s, 3H). ¹H NMR (400 MHz, DMSO-d₆) of the minor diastereoisomer: δ 10.92 (bs, 1H), 8.62 (d, J=4.7 Hz, 1H), 8.18 (ddd, J=8.0, 4.7, 1.6 Hz, 1H), 7.56 (dd, J=8.0, 4.7 Hz, 1H), 6.96 (s, 1H), 6.26 (d, J=3.1 Hz, 1H), 5.28 (d, J=3.1 Hz, 1H), 2.43 (s, 3H).

### Intermediate N23: 3-Fluoro-5,10-dimethyl-4,8,12-triazatricyclo[9.4.0.0^{2,7}]pentadeca-1(11),2(7),3,5,12,14-hexaen-9-one

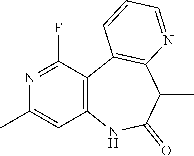

### Step 1: Synthesis of methyl 2-[3-(4-amino-2-fluoro-6-methyl-3-pyridyl)-2-pyridyl]propanoate N23_1

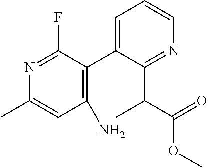 [SMILES: COC(=O)C(C)c1ncccc1-c1c(N)cc(C)nc1F]

To a solution of 2-fluoro-6-methyl-3-(4,4,5,5-tetramethyl-1,3,2-dioxaborolan-2-yl)pyridin-4-amine (Intermediate N16_1, 100 mg, 0.39 mmol) and methyl 2-(3-bromopyridin-2-yl)propanoate (102 mg, 0.40 mmol) in dry toluene (2 mL) was added potassium carbonate (170 mg, 1.22 mmol). Argon was passed throughout the reaction mixture before addition of tris(dibenzylideneacetone)dipalladium(0) (36 mg, 0.04 mmol) and 2-dicyclohexylphosphino-2,6-dimethoxybiphenyl (17 mg, 0.04 mmol). The reaction mixture was then stirred at 100° C. for 4 h. After cooling to room temperature, the reaction mixture was diluted with EtOAc (10 mL) and filtered through a PTFE filter. The filtrate was concentrated under vacuum to afford the crude title compound as a yellow oil (225 mg) which was directly used in the next step without purification. LC-MS (Method A1) m/z: [M+H]⁺: 290; rt: 0.80, 0.84 and 0.92 min.

### Step 2: Synthesis of 3-fluoro-5,10-dimethyl-4,8,12-triazatricyclo[9.4.0.0^{2,7}]pentadeca-1(11),2(7),3,5,12,14-hexaen-9-one N23

At 0° C., to a solution of crude methyl 2-[3-(4-amino-2-fluoro-6-methyl-3-pyridyl)-2-pyridyl]propanoate (Intermediate N23_1, 115 mg, 0.40 mmol) in dry toluene (2 mL) was added a 1.5 M solution of lithium bis(trimethylsilyl)amide in THF (200 μL, 0.30 mmol) and the resulting mixture was stirred at room temperature for 30 min. The reaction mixture was neutralized at 0° C. by the addition of saturated aqueous NH₄Cl and extracted with EtOAc (3×20 mL). The combined organic layers were washed with brine (20 mL), dried over MgSO₄, filtered off and concentrated under vacuum to give a yellow solid. Purification by trituration in Et₂O afforded the title compound as a brown solid (11 mg, yield: 10%). LC-MS (Method A1) m/z: [M+H]⁺: 258.0; rt: 0.93 min; purity: 94%.

### Intermediate N24: 14-Methoxy-12-methyl-4,5,6,9,13-pentazatricyclo[8.4.0.0^{2,6}]tetradeca-1(10),2,4,11,13-pentaen-8-one

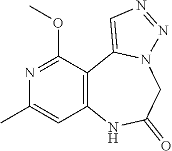 [SMILES: COc1nc(C)cc2c1-c1cnnn1CC(=O)N2]

### Step 1: Synthesis of 3-iodo-2-methoxy-6-methyl-pyridin-4-amine N24_1

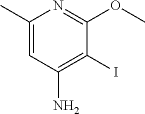

2-Methoxy-6-methyl-pyridin-4-amine (2.5 g, 18.1 mmol) was dissolved in dry THF (100 mL) and N-iodosuccinimide (3.66 g, 16.3 mmol) was added portionwise over 45 min at −78° C. The reaction mixture was stirred at −78° C. for 3 h and then allowed to gradually warm to room temperature. The resultant suspension was diluted with water (50 mL), extracted with ethyl acetate (3×50 mL) and the combined organic extracts were washed with brine (2×50 mL). The organic phase was filtered through a hydrophobic frit and concentrated under vacuum. The residue was purified by flash chromatography on silica gel (using 0 to 30% ethyl acetate in cyclohexane as eluent) to give the title compound (4.59 g, yield: 93%) as a pale orange solid. LC-MS (Method B5) m/z: [M+H]⁺: 265.0; rt: 1.69 min, purity: 99%. ¹H NMR (400 MHz, CDCl₃) δ 6.14 (d, J=0.7 Hz, 1H), 4.54 (s, 2H), 3.95 (s, 3H), 2.32 (d, J=0.7 Hz, 3H).

### Step 2: Synthesis of 2-methoxy-6-methyl-3-(2-trimethylsilylethynyl)pyridin-4-amine N24_2

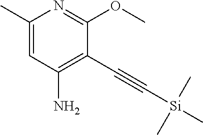

A mixture of 3-iodo-2-methoxy-6-methyl-pyridin-4-amine (Intermediate N24_1, 5.00 g, 18.9 mmol), trimethylsilylacetylene (11.16 g, 114 mmol), cuprous iodide (361 mg, 1.89 mmol), bis(triphenylphosphine)palladium(II) chloride (1.33 g, 1.89 mmol) and triethylamine (18.5 mL, 133 mmol) in dry dioxane (70 mL) was heated at 80° C. for 2 h. The reaction mixture was cooled to room temperature and filtered through a small plug of Celite®. The filter cake was rinsed with ethyl acetate (70 mL) and the filtrate was concentrated under vacuum. The residue was purified by flash chromatography on silica gel (using 0 to 30% ethyl acetate in cyclohexane as eluent) to give the title compound (2.80 g, yield: 57%) as an orange oil. ¹H NMR (400 MHz, CDCl₃) δ 6.13 (d, J=0.7 Hz, 1H), 4.63 (s, 2H), 3.98 (s, 3H), 2.33 (s, 3H), 0.29 (s, 9H).

### Step 3: Synthesis of 3-ethynyl-2-methoxy-6-methyl-pyridin-4-amine N24_3

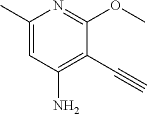

2-Methoxy-6-methyl-3-(2-trimethylsilylethynyl)pyridin-4-amine (Intermediate N24_2, 2.50 g, 10.1 mmol) was dissolved in MeOH (50 mL) and potassium carbonate (1.68 g, 12.2 mmol) was added at 0° C. The reaction mixture was stirred at 0° C. for 30 min and at room temperature for 2 h. The reaction mixture was concentrated under vacuum and the residue was partitioned between ethyl acetate (50 mL) and water (50 mL). The layers were separated, and the aqueous layer was extracted with ethyl acetate (2×30 mL). The combined organic layers were filtered through a hydrophobic frit and concentrated under vacuum. The residue was purified by flash chromatography on silica gel (using 0 to 100% ethyl acetate in iso-hexane as eluent) to give the title compound (1.35 g, yield: 80%) as a yellow oil. LC-MS (Method B5) m/z: [M+H]⁺: 163.0; rt: 1.35 min, purity: 97%. ¹H NMR (400 MHz, CDCl₃) δ 6.13 (s, 1H), 4.64 (s, 2H), 3.99 (s, 3H), 3.63 (s, 1H), 2.34 (s, 3H).

### Step 4: Synthesis of 2-chloro-N-(3-ethynyl-2-methoxy-6-methyl-4-pyridyl)acetamide N24_4

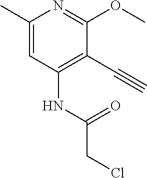

3-Ethynyl-2-methoxy-6-methyl-pyridin-4-amine (Intermediate N24_3, 1.25 g, 7.40 mmol) and triethylamine (973 mg, 9.62 mmol) were dissolved in dry DCM (25 mL) and 2-chloroacetyl chloride (1.67 g, 14.8 mmol) was slowly added at 0° C. The reaction mixture was allowed to gradually warm up to room temperature and stirred for 20 h. The reaction mixture was diluted with DCM (25 mL) and washed with saturated aqueous NaHCO₃ (2×20 mL). The organic phase was filtered through a hydrophobic frit and concentrated under vacuum to give the title compound (1.90 g, yield: 97%) as a brown solid. The product was taken to the next step without further purification nor analysis.

### Step 5: Synthesis of 2-azido-N-(3-ethynyl-2-methoxy-6-methyl-4-pyridyl)acetamide N24_5

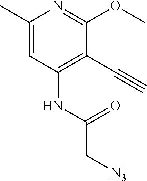

2-Chloro-N-(3-ethynyl-2-methoxy-6-methyl-4-pyridyl)acetamide (Intermediate N24_4, 1.90 g, 7.16 mmol) was dissolved in dry DMF (20 mL) and sodium azide (699 mg, 10.7 mmol) was added. The reaction mixture was stirred at room temperature for 3 h, before being diluted with water (40 mL) and extracted with ethyl acetate (3×30 mL). The combined organic extracts were filtered through a hydrophobic frit and concentrated under vacuum. The residue was purified by flash chromatography on silica gel (using 0 to 50% ethyl acetate in iso-hexane as eluent) to give the title compound (1.64 g, yield: 87%) as a light orange solid. LC-MS (Method B5) m/z: [M+H]⁺: 246.0; rt: 1.93 min, purity: 93%. ¹H NMR (400 MHz, CDCl₃) δ 9.00 (s, 1H), 7.88 (s, 1H), 4.20 (s, 2H), 4.04 (s, 3H), 3.82 (s, 1H), 2.48 (s, 3H).

### Step 6: Synthesis of 14-methoxy-12-methyl-4,5,6,9,13-pentazatricyclo[8.4.0.0^{2,6}]tetradeca-1(10),2,4,11,13-pentaen-8-one N24

2-Azido-N-(3-ethynyl-2-methoxy-6-methyl-4-pyridyl)acetamide (Intermediate N24_5, 1.64 g, 6.22 mmol) was dissolved in dry DMF (120 mL) and stirred at 150° C. for 18 h. The reaction mixture was cooled to room temperature and diluted with water (150 mL). The aqueous phase was extracted with ethyl acetate (3×150 mL) and the combined organic layers were washed with brine (2×100 mL). The organic phase was filtered through a hydrophobic frit and concentrated under vacuum. The residue was triturated in MeOH (50 mL) and filtered. The filtrate was concentrated under vacuum and triturated in tertbutylmethylether (50 mL). The solid thus formed was collected by filtration and dissolved in MeOH (50 mL) to form a suspension. the suspension was filtered, and the filtrate was concentrated under vacuum to give the title compound (1.51 g, yield: 69%) as a beige solid. LC-MS (Method B5) m/z: [M+H]⁺: 246.0; rt: 1.20 min, purity: 80%. ¹H NMR (400 MHz, DMSO-d₆) δ 11.04 (s, 1H), 8.06 (s, 1H), 6.77 (s, 1H), 5.20 (s, 2H), 3.96 (s, 3H), 2.42 (s, 3H).

### Intermediate N25: 3-methoxy-5-methyl-4,8,12-triazatricyclo[9.4.0.0^{2,7}]pentadeca-1(11),2(7),3,5,12,14-hexaen-9-one

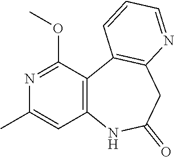 [SMILES: COc1nc(C)cc2c1-c1cccnc1CC(=O)N2]

### Step 1: Synthesis of 3-bromo-2-methoxy-6-methyl-pyridin-4-amine N25_1

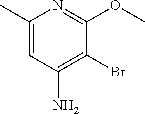

At 0° C., to a solution of 2-methoxy-6-methyl-pyridin-4-amine (505 mg, 3.47 mmol) in DCM (25 mL) was added a suspension of NBS (618 mg, 3.47 mmol) in DCM (10 mL) and the reaction mixture was stirred at 0° C. for 1 h. Water (40 mL) was added and the aqueous layer was extracted with DCM (2×30 mL). The combined organic extracts were washed with brine (80 mL), dried over Na₂SO₄, filtered and concentrated under vacuum. The residue was purified by flash chromatography on silica gel (using 0 to 4% MeOH (0.7 N in NH₃) in DCM as eluent) to give the title compound as a colorless oil (653 mg, yield: 86%). LC-MS (Method B5) m/z: [M+H]⁺: 217.1/219.1; rt: 1.57 min; purity>99%. ¹H NMR (400 MHz, CDCl₃) δ 6.16 (d, J=0.7 Hz, 1H), 4.47 (s, 2H), 3.97 (s, 3H), 2.31 (s, 3H).

### Step 2: Synthesis of ethyl 2-[3-(4,4,5,5-tetramethyl-1,3,2-dioxaborolan-2-yl)-2-pyridyl]acetate N25_2

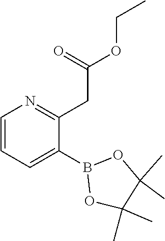 [SMILES: CCOC(=O)Cc1ncccc1B1OC(C)(C)C(C)(C)O1]

Under inert atmosphere, to a suspension of ethyl 2-(3-bromo-2-pyridyl)acetate (Intermediate N3_1, 5.00 g, 20.5 mmol), bis(pinacolato)diboron (CAS 73183-34-3, 6.24 g, 24.6 mmol), potassium acetate (8.0 g, 81.9 mmol), 3A molecular sieves (3 g) in 1,4-dioxane (200 mL), was added [1,1′-bis(diphenylphosphino)ferrocene]dichloropalladium(II) (749 mg, 1.02 mmol). The suspension was heated at reflux overnight then cooled to room temperature. It was filtered on a pad of Celite® and washed with ethyl acetate (400 mL). The filtrate was concentrated under vacuum and the residue was purified by flash chromatography on silica gel (using 0 to 100% tertbutylmethylether in cyclohexane as eluent) to give the title compound as a brown oil. (2.35 g, yield: 37%). LC-MS (Method B5) m/z: [M+H]⁺: 292.2; rt: 2.08 min, purity: 95%. ¹H NMR (400 MHz, CDCl₃) δ 8.60 (dd, J=4.9, 1.9 Hz, 1H), 8.14 (dd, J=7.6, 1.9 Hz, 1H), 7.23 (dd, J=7.6, 4.9 Hz, 1H), 4.22 (s, 2H), 4.18 (q, J=7.1 Hz, 2H), 1.35 (s, 12H), 1.28-1.24 (m, 3H).

### Step 3: Synthesis of 3-methoxy-5-methyl-4,8,12-triazatricyclo[9.4.0.0^{2,7}]pentadeca-1(11),2(7),3,5,12,14-hexaen-9-one N25

Under inert atmosphere, to a suspension of 3-bromo-2-methoxy-6-methyl-pyridin-4-amine (Intermediate N25_1, 550 mg, 2.51 mmol), ethyl 2-[3-(4,4,5,5-tetramethyl-1,3,2-dioxaborolan-2-yl)-2-pyridyl]acetate (Intermediate N25_2, 1.25 g, 3.51 mmol), SPhos (103 mg, 0.251 mmol) and CsF (1.07 g, 7.02 mmol) in 1,4-dioxane (22.8 mL) and water (1.1 mL) was added palladium(II) acetate (28.2 mg, 0.125 mmol). The solution was heated at reflux overnight. It was cooled to room temperature and filtered through a pad of Celite®, washed with ethyl acetate (150 mL) and the filtrate was concentrated under vacuum. The residue was dissolved in EtOH (22.8 mL) and potassium carbonate (693 mg, 5.02 mmol) was added. The reaction mixture was heated at reflux overnight and EtOH was removed under vacuum. Water (50 mL) was added and the mixture was extracted with ethyl acetate (3×70 mL). The combined organic extracts were washed with brine (100 mL), dried over Na₂SO₄, filtered and concentrated under vacuum. The residue was purified by flash chromatography on silica gel (using 0 to 10% MeOH (0.7N NH₃) in DCM as eluent). The fractions were evaporated, and the resulting solid was triturated with tertbutylmethylether to give the title compound as an off-white solid (378 mg, yield: 59%). LC-MS (Method B5) m/z: [M+H]⁺: 256.2; rt: 1.45 min, purity>99%. ¹H NMR (400 MHz, DMSO-d₆) δ 10.45 (s, 1H), 8.51 (dd, J=4.7, 1.7 Hz, 1H), 8.14 (dd, J=8.0, 1.7 Hz, 1H), 7.40 (dd, J=8.0, 4.7 Hz, 1H), 6.70 (s, 1H), 3.89 (s, 3H), 3.65 (d, J=12.4 Hz, 1H), 3.59 (d, J=12.4 Hz, 1H), 2.41 (s, 3H).

### Intermediate N26: 15-methoxy-13-methyl-4,6,10,14-tetrazatricyclo[9.4.0.0^{2,7}]pentadeca-1(11),2(7),3,5,12,14-hexaen-9-one

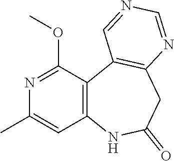 [SMILES: COc1nc(C)cc2c1-c1cncnc1CC(=O)N2]

### Step 1: Synthesis of ethyl 2-[5-(4,4,5,5-tetramethyl-1,3,2-dioxaborolan-2-yl)pyrimidin-4-yl]acetate N26_1

To a solution of ethyl 2-(5-bromopyrimidin-4-yl)acetate (Intermediate N36_2, 0.10 g, 0.408 mmol) in dioxane (2 mL), potassium acetate (120 mg, 1.22 mmol) and bis(pinacolato)diboron (155 mg, 0.612 mmol) were added. The reaction mixture was purged with nitrogen for 5 min and 1,1′-bis(diphenylphosphino)ferrocene-palladium(II)dichloride dichloromethane complex (33 mg, 0.041 mmol) was added. The reaction mixture was heated at 80° C. for 18 h. The reaction mixture was cooled to room temperature and filtered through a small pad of Celite®. The filter cake was rinsed with ethyl acetate (20 mL) and the filtrate was concentrated under vacuum to afford the crude title compound as a tan solid which was taken to the next step without purification.

### Step 2: Synthesis of 15-methoxy-13-methyl-4,6,10,14-tetrazatricyclo[9.4.0.0^{2,7}]pentadeca-1(11),2(7),3,5,12,14-hexaen-9-one N26

To a solution of 3-bromo-2-methoxy-6-methyl-pyridin-4-amine (Intermediate N25_1, 80 mg, 0.365 mmol) in dioxane (2 mL) and water (0.1 mL), ethyl 2-[5-(4,4,5,5-tetramethyl-1,3,2-dioxaborolan-2-yl)pyrimidin-4-yl]acetate (107 mg, 0.365 mmol) and CsF (166 mg, 1.09 mmol) were added. The reaction mixture was purged with nitrogen for 5 min and S-Phos (15 mg, 0.036 mmol) and palladium(II) acetate (8 mg, 0.036 mmol) were added. The reaction mixture was purged again with nitrogen for 5 min and heated at 90° C. for 18 h. The reaction mixture was cooled to room temperature and filtered through a small pad of Celite®. The filter cake was rinsed with ethyl acetate (20 mL) and the filtrate was concentrated under vacuum. The residue was dissolved in ethanol (2 mL), potassium carbonate (101 mg, 0.73 mmol) was added and the reaction mixture was heated at 70° C. for 4 h. After cooling to room temperature, the reaction mixture was filtered on Celite®, washed with ethyl acetate (15 mL) and the filtrate was concentrated under vacuum. The residue was purified by flash chromatography on silica gel (using 0 to 10% MeOH in DCM as eluent) to give the title compound (25 mg, yield: 23%) as a light yellow solid. LC-MS (Method B5) m/z [M+H]⁺: 257.2; rt: 1.30 min; purity 86%. ¹H NMR (400 MHz, DMSO-d₆) δ 10.66 (s, 1H), 9.16 (s, 1H), 9.09 (s, 1H), 6.73 (s, 1H), 3.92 (s, 3H), 3.81 (d, J=12.5 Hz, 1H), 3.55 (d, J=12.5 Hz, 1H), 2.43 (s, 3H).

### Intermediate N27: 14-methoxy-12-methyl-5,6,9,13-tetrazatricyclo[8.4.0.0^{2,6}]tetradeca-1(10),2,4,11,13-pentaen-8-one

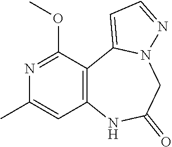 [SMILES: COc1nc(C)cc2c1-c1ccnn1CC(=O)N2]

### Step 1: Synthesis of 2-methoxy-6-methyl-3-(2-tetrahydropyran-2-ylpyrazol-3-yl)pyridin-4-amine N27_1

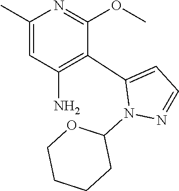 [SMILES: COc1nc(C)cc(N)c1-c1ccnn1C1CCCCO1]

A mixture of 3-bromo-2-methoxy-6-methyl-pyridin-4-amine (Intermediate N25_1, 2.00 g, 9.21 mmol), 1-tetrahydropyran-2-yl-5-(4,4,5,5-tetramethyl-1,3,2-dioxaborolan-2-yl)pyrazole (3.07 g, 11.1 mmol), bis(dibenzylideneacetone)palladium (265 mg, 0.461 mmol), S-Phos (189 mg, 0.461 mmol) and potassium carbonate (3.82 g, 27.6 mmol) in dry dioxane (45 mL) was heated at 90° C. for 3 h. The reaction mixture was cooled to room temperature and filtered through a small pad of Celite®. The filter cake was rinsed with ethyl acetate (45 mL) and the filtrate was concentrated under vacuum. The residue was purified by flash chromatography on silica gel (using 0 to 100% ethyl acetate in iso-hexane) to give the title compound (1.10 g, yield: 38%) as a sticky orange solid. LC-MS (Method B5) m/z: [M+H]⁺: 289.0; rt: 1.59 min; purity: 91%. ¹H NMR (400 MHz, DMSO-d₆) δ 7.58 (d, J=1.9 Hz, 1H), 6.23 (d, J=12.9 Hz, 1H), 6.18 (dd, J=18.0, 1.7 Hz, 1H), 5.46 (d, J=25.7 Hz, 2H), 4.94-4.80 (m, 1H), 3.89-3.80 (m, 1H), 3.67 (m, 3H), 3.39-3.33 (m, 1H), 2.34-2.20 (m, 4H), 1.98-1.91 (m, 1H), 1.82-1.72 (m, 1H), 1.63-1.42 (m, 3H). Split peaks due to atropisomerism.

### Step 2: Synthesis of 2-chloro-N-[2-methoxy-6-methyl-3-(1H-pyrazol-5-yl)-4-pyridyl]acetamide N27_2

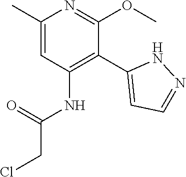 [SMILES: COc1nc(C)cc(NC(=O)CCl)c1-c1ccn[nH]1]

At 0° C., 2-methoxy-6-methyl-3-(2-tetrahydropyran-2-ylpyrazol-3-yl)pyridin-4-amine (Intermediate N27_1, 1.05 g, 3.46 mmol) was dissolved in dry THF (25 mL) and sodium hydride (60% dispersion in oil, 208 mg, 5.19 mmol) was added. The resulting suspension was stirred at room temperature for 15 min and then 2-chloroacetyl chloride (0.55 mL, 6.92 mmol) was slowly added at 0° C. The reaction mixture was allowed to gradually warm up to room temperature and stirred overnight. The reaction mixture was diluted with water (30 mL) and saturated aqueous NaHCO₃ (30 mL) and extracted with ethyl acetate (3×40 mL). The combined organic extracts were filtered through a hydrophobic frit and concentrated under vacuum. The residue was purified by flash chromatography on silica gel (using 0 to 100% ethyl acetate in iso-hexane as eluent) to give the title compound (427 mg, yield: 36%) as a yellow solid. LC-MS (Method B5) m/z: [M+H]⁺: 281.0/283.0; rt: 1.77 min; purity: 83%.

### Step 3: Synthesis of 14-methoxy-12-methyl-5,6,9,13-tetrazatricyclo[8.4.0.0^{2,6}]tetradeca-1(10),2,4,11,13-pentaen-8-one N27

2-Chloro-N-[2-methoxy-6-methyl-3-(1H-pyrazol-5-yl)-4-pyridyl]acetamide (intermediate N27_2, 427 mg, 1.26 mmol) was dissolved in dry DMF (15 mL) and sodium hydride (60% dispersion in oil, 76 mg, 1.89 mmol) was added. The reaction mixture was stirred at room temperature for 1 h. Water (35 mL) was added and the mixture was extracted with ethyl acetate (2×20 mL). The combined organic extracts were washed with brine (2×20 mL), filtered through a hydrophobic frit and concentrated under vacuum. The residue was purified by flash chromatography on silica gel (using 0 to 10% MeOH in DCM as eluent) to give the title compound (245 mg, yield: 71%) as a light yellow solid. LC-MS (Method B5) m/z: [M+H]⁺: 245.0; rt: 1.39 min; purity: 89%. ¹H NMR (400 MHz, DMSO-d₆) δ 10.73 (s, 1H), 7.56 (d, J=2.0 Hz, 1H), 6.68 (s, 1H), 6.67 (d, J=2.0 Hz, 1H), 4.85 (s, 2H), 3.93 (s, 3H), 2.40 (s, 3H).

### Intermediate N28: 3,10-difluoro-5-methyl-4,8,12-triazatricyclo[9.4.0.0^{2,7}]pentadeca-1(11),2(7),3,5,12,14-hexaen-9-one

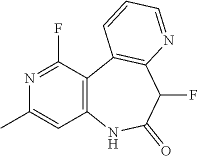 [SMILES: Cc1cc2c(c(F)n1)-c1cccnc1C(F)C(=O)N2]

### Step 1: Synthesis of ethyl 2-(3-bromo-2-pyridyl)-2-fluoro-acetate N28_1

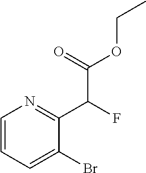

A solution of ethyl 2-(3-bromo-2-pyridyl)acetate (Intermediate N3_1, 100 mg, 0.40 mmol) and 1-chloromethyl-4-fluoro-1,4-diazoniabicyclo[2.2.2]octane bis(tetrafluoroborate) (Selectfluor®, 165 mg, 0.44 mmol) in acetonitrile (0.9 mL) and water (0.9 mL) was stirred at room temperature for 16 h. The reaction mixture was extracted twice with EtOAc, washed with brine, dried over MgSO₄, filtered off and concentrated under vacuum to give the title product as a colorless oil (88 mg, yield: 84%). LC-MS (Method A1) m/z: [M+H]P: 264.0, rt: 1.25 min. purity: 99%. ¹H NMR (400 MHz, DMSO-d₆) δ 8.62 (dd, J=4.6, 1.4 Hz, 1H), 8.24 (dt, J=8.1, 1.4 Hz, 1H), 7.48 (ddd, J=8.1, 4.6, 1.4 Hz, 1H), 6.43 (d, J=46.7 Hz, 1H), 4.22 (q, J=7.1 Hz, 2H), 1.18 (t, J=7.1 Hz, 3H).

### Step 2: Synthesis of ethyl 2-[3-(4-amino-2-fluoro-6-methyl-3-pyridyl)-2-pyridyl]-2-fluoro-acetate

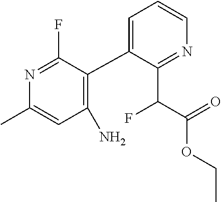 [SMILES: CCOC(=O)C(F)c1ncccc1-c1c(N)cc(C)nc1F]

The title compound was prepared following the same procedure as for intermediate N22, step 2, starting from 2-fluoro-6-methyl-3-(4,4,5,5-tetramethyl-1,3,2-dioxaborolan-2-yl)pyridin-4-amine (Intermediate N16_1, 100 mg, 0.36 mmol) and ethyl 2-(3-bromo-2-pyridyl)-2-fluoro-acetate (intermediate N28_1, 97 mg, 0.36 mmol). The reaction mixture was diluted with EtOAc and filtered over Celite®. The filtrate was concentrated to dryness and taken crude to the next step (109 mg, yield: 99%). LC-MS (Method B1) m/z: [M+H]⁺: 309.0, rt: 1.12 min.

### Step 3: Synthesis of 3,10-difluoro-5-methyl-4,8,12-triazatricyclo[9.4.0.02 ⁷]pentadeca-1(11),2(7),3,5,12,14-hexaen-9-one N28

The title compound was prepared following the same procedure as for intermediate N22, step 3, starting from ethyl 2-[3-(4-amino-2-fluoro-6-methyl-3-pyridyl)-2-pyridyl]-2-fluoro-acetate (intermediate N28_2, 109 mg, 0.35 mmol). Purification by trituration in diethyl ether afforded the title compound (52 mg, yield: 47%). LC-MS (Method B1) m/z: [M+H]⁺: 262.0, rt: 0.83 min, purity: 84%.

### Intermediate N29: 3-fluoro-5-methyl-4,8,11-triazatricyclo[9.4.0.0^{2,7}]pentadeca-1(15),2(7),3,5,13-pentaene-9,12-dione

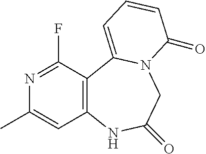 [SMILES: Cc1cc2c(c(F)n1)-c1cccc(=O)n1CC(=O)N2]

### Step 1: Synthesis of methyl 2-(2-bromo-6-oxo-1-pyridyl)acetate N29_1

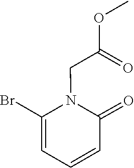

At 0° C., to a solution of 6-bromopyridin-2(1H)-one (100 mg, 0.55 mmol) in dry 1,2-dimethoxyethane (1 mL) and dry DMF (0.3 mL) was added sodium hydride (60% dispersion in oil, 24 mg, 0.65 mmol) and the reaction mixture was stirred for 10 min. Then, lithium bromide (104 mg, 1.20 mmol) was added in one portion and the resulting mixture was stirred at room temperature for 15 min. Methyl bromoacetate (78 μL, 0.82 mmol) was added and the reaction mixture was heated at 60° C. for 16 h. The reaction mixture was poured into cold water and extracted with DCM (3×10 mL). The combined organic layers were dried over MgSO₄, filtered off and concentrated under vacuum to give a crude colorless oil. Purification by column chromatography on silica gel (using a gradient of heptane/EtOAc from 100:0 to 50:50 as eluent) afforded the title compound as a colorless oil (60 mg, yield: 45%). LC-MS (Method A1) m/z: [M+H]⁺: 246.0, rt: 0.87 min, purity: 99%. ¹H NMR (400 MHz, DMSO-d₆) δ 7.38 (dd, J=9.2, 7.2 Hz, 1H), 6.69 (dd, J=7.2, 1.2 Hz, 1H), 6.47 (dd, J=9.2, 1.2 Hz, 1H), 4.99 (s, 2H), 3.71 (s, 3H).

### Step 2: Synthesis of 3-fluoro-5-methyl-4,8,11-triazatricyclo[9.4.0.0^{2,7}]pentadeca-1(15),2(7),3,5,13-pentaene-9,12-dione N29

To a solution of 2-fluoro-6-methyl-3-(4,4,5,5-tetramethyl-1,3,2-dioxaborolan-2-yl)pyridin-4-amine (Intermediate N16_1, 100 mg, 0.40 mmol) and methyl 2-(2-bromo-6-oxo-1-pyridyl)acetate (98 mg, 0.40 mmol) in dry toluene (2 mL) was added K₃PO₄ (260 mg, 1.20 mmol). Argon was passed throughout the reaction mixture before addition of both tris(dibenzylideneacetone)dipalladium(0) (36 mg, 0.04 mmol) and 2-dicyclohexylphosphino-2,6-dimethoxybiphenyl (17 mg, 0.04 mmol). The reaction mixture was then stirred at 100° C. for 10 h. After cooling to room temperature, the reaction mixture was diluted with EtOAc and filtered over Celite®. The filtrate was concentrated under vacuum. Purification by trituration with Et₂O afforded the title compound as a dark green solid (58 mg, yield: 46%). LC-MS (Method B1) m/z: [M+H]⁺: 259.9, rt: 0.79 min, purity: 81%.

### Intermediate N30: 14-chloro-4,12-dimethyl-5,6,9,13-tetrazatricyclo[8.4.0.0^{2,6}]tetradeca-1(14),2,4,10,12-pentaen-8-one

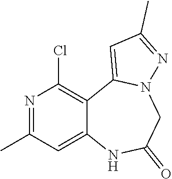 [SMILES: Cc1cc2c(c(Cl)n1)-c1cc(C)nn1CC(=O)N2]

### Step 1: Synthesis of 3-bromo-2-chloro-6-methyl-pyridin-4-amine N30_1

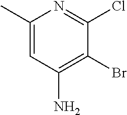

A solution of 2-chloro-6-methyl-pyridin-4-amine (10.0 g, 66.6 mmol) in dry acetonitrile (300 mL) was cooled to 0° C., then N-bromosuccinimide (11.3 g, 63.3 mmol) was added over 1 h and the reaction mixture was stirred at 0° C. for 3 h, then warmed to room temperature and stirred for a further 18 h. The reaction mixture was then concentrated under vacuum and purified by column chromatography on silica gel (using a gradient of 0 to 50% EtOAc in iso-hexane as eluent) to afford the title compound (7.05 g, yield: 47%) as a yellow solid. ¹H NMR (400 MHz, DMSO-d₆) δ 6.53 (s, 2H), 6.47 (d, J=0.7 Hz, 1H), 2.20 (s, 3H).

### Step 2: Synthesis of 3-methyl-1-tetrahydropyran-2-yl-pyrazole N30_2

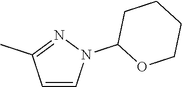

A stirring solution of 3-methyl-1H-pyrazole (10.0 g, 122 mmol), 2,3-dihydro-4H-pyran (13.3 mL, 146 mmol) and TFA (0.45 mL, 6.09 mmol) in dry toluene (70 mL) was heated at 110° C. for 24 hours. The reaction mixture was cooled to room temperature and diluted with brine (100 mL) and saturated aqueous NaHCO₃ (100 mL). The aqueous layer was extracted with EtOAc (3×100 mL) and the combined organic layers were filtered through a hydrophobic frit then concentrated under vacuum. The residue was purified by flash chromatography on silica gel (using a gradient of 0 to 50% MTBE in iso-hexane as eluent) to afford the title compound (20.3 g, yield: 92%) as a colourless oil. LC-MS (Method B5′) m/z: [M+H]⁺: 167.0; rt: 1.23 min; purity: 99%.

### Step 3: Synthesis of 3-methyl-1-tetrahydropyran-2-yl-5-(4,4,5,5-tetramethyl-1,3,2-dioxaborolan-2-yl)pyrazole N30_3

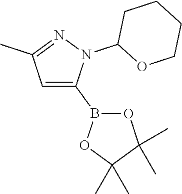 [SMILES: Cc1cc(B2OC(C)(C)C(C)(C)O2)n(C2CCCCO2)n1]

3-Methyl-1-tetrahydropyran-2-yl-pyrazole (intermediate N30_2, 9.50 g, 57.2 mmol) was dissolved in dry THF (70 mL) and n-butyllithium (2.50 M in hexanes, 25 mL, 62.9 mmol) was then slowly added at −78° C. The reaction mixture was stirred at −78° C. for 1.5 hours. Triisopropyl borate (11.8 g, 62.9 mmol) was then slowly added at −78° C. and the reaction mixture was stirred at −78° C. for a further 15 min. The reaction mixture was allowed to gradually warm to room temperature then stirred for a further 1.5 hours. Pinacol (7.43 g, 62.9 mmol) and acetic acid (6.54 mL, 114 mmol) were added and the reaction mixture was stirred at room temperature for 18 hours. The reaction mixture was concentrated under vacuum and the residue was partitioned between water (150 mL) and EtOAc (150 mL). The layers were separated, and the aqueous layer was extracted with EtOAc (100 mL). The combined organic layers were washed with brine (2×50 mL), filtered through a hydrophobic frit and concentrated under vacuum. The residue was purified by flash chromatography on silica gel (using a gradient of 0 to 50% MTBE in iso-hexane as eluent) to afford the title compound (11.20 g, yield: 64%) as a light-yellow oil. ¹H NMR (400 MHz, CDCl₃) δ 6.54 (s, 1H), 5.79 (dd, J=10.5, 2.3 Hz, 1H), 4.11-4.05 (m, 1H), 3.74-3.61 (m, 1H), 2.55-2.40 (m, 1H), 2.32 (s, 3H), 2.13-2.06 (m, 1H), 1.98-1.92 (m, 1H), 1.73 (ddt, J=25.6, 12.7, 3.9 Hz, 2H), 1.59-1.50 (m, 1H), 1.35 (s, 12H).

### Step 4: Synthesis of 2-chloro-6-methyl-3-(5-methyl-2-tetrahydropyran-2-yl-pyrazol-3-yl)pyridin-4-amine N30_4

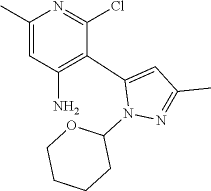

A stirring mixture of 3-bromo-2-chloro-6-methyl-pyridin-4-amine (intermediate N30_1, 0.40 g, 1.81 mmol), 3-methyl-1-tetrahydropyran-2-yl-5-(4,4,5,5-tetramethyl-1,3,2-dioxaborolan-2-yl)pyrazole (Intermediate N30_3, 1.06 g, 3.61 mmol), bis(di-tert-butyl(4-dimethylaminophenyl)-phosphine)-dichloro-palladium(II) (128 mg, 0.181 mmol) and aqueous potassium carbonate (1.50 M, 3.6 mL, 5.42 mmol) in 1,4-dioxane (40 mL) was heated at 90° C. for 1.5 hours. The reaction mixture was cooled to room temperature and diluted with EtOAc (40 mL). The resulting suspension was filtered through a small pad of Celite® and the filter cake was rinsed with EtOAc (30 mL). The filtrate was concentrated under vacuum and the residue was purified by flash chromatography on silica gel (using a gradient of 0 to 100% EtOAc in iso-hexane as eluent) to afford the title compound (405 mg, yield: 68%) as a yellow foam. Product obtained as a mixture of diastereomers. LC-MS (Method B5′) m/z: [M+H]⁺: 307/309; rt: 1.25/1.47 min; purity: 94%. ¹H NMR (400 MHz, CDCl₃) δ 6.45 (t, J=1.2 Hz, 1H), 6.12-6.07 (m, 1H), 4.89 (dd, J=10.6, 2.4 Hz, 1H), 4.42 (s, 2H), 4.08-3.96 (m, 1H), 3.57-3.40 (m, 1H), 2.59-2.44 (m, 4H), 2.39-2.34 (m, 3H), 2.04-1.92 (m, 2H), 1.81-1.46 (m, 3H).

### Step 5: Synthesis of 2-chloro-N-[2-chloro-6-methyl-3-(5-methyl-2-tetrahydropyran-2-yl-pyrazol-3-yl)-4-pyridyl]acetamide N30_5

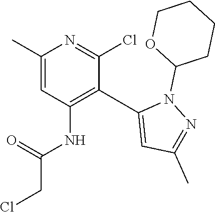 [SMILES: Cc1cc(NC(=O)CCl)c(-c2cc(C)nn2C2CCCCO2)c(Cl)n1]

2-Chloro-6-methyl-3-(5-methyl-2-tetrahydropyran-2-yl-pyrazol-3-yl)pyridin-4-amine (intermediate N30_4, 400 mg, 1.21 mmol) was dissolved in dry DCM (15 mL) and N,N-diisopropylethylamine (940 mg, 7.28 mmol) was added. 2-Chloroacetyl chloride (0.24 mL, 3.03 mmol) was slowly added dropwise at 0° C. and the reaction mixture was allowed to gradually warm to room temperature and stirred overnight. The reaction mixture was diluted with DCM (25 mL) and then washed with water (2×25 mL). The organic phase was filtered through a hydrophobic frit and concentrated under vacuum. The residue was purified by flash chromatography on silica gel (using a gradient of 0 to 100% EtOAc in iso-hexane as eluent) to afford the title compound (122 mg, yield: 23%) as an orange oil. ¹H NMR (400 MHz, CDCl₃) δ 8.44 (s, 1H), 8.16 (d, J=18.2 Hz, 1H), 6.16 (d, J=12.0 Hz, 1H), 4.81 (dd, J=10.2, 2.5 Hz, 1H), 4.04 (d, J=8.4 Hz, 2H), 3.98-3.90 (m, 1H), 3.34 (m, 1H), 2.64 (d, J=1.2 Hz, 3H), 2.61-2.48 (m, 1H), 2.38 (d, J=9.8 Hz, 3H), 2.05-1.92 (m, 2H), 1.76-1.43 (m, 3H).

### Step 6: Synthesis of 13-chloro-7-(chloromethyl)-4,11-dimethyl-5,6,8,12-tetrazatricyclo[7.4.0.0^{2,6}]trideca-1(13),2,4,7,9,11-hexaene hydrochloride N30 6

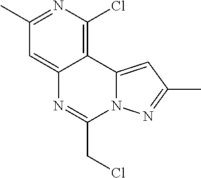

2-Chloro-N-[2-chloro-6-methyl-3-(5-methyl-2-tetrahydropyran-2-yl-pyrazol-3-yl)-4-pyridyl]acetamide (intermediate N30_5, 122 mg, 0.28 mmol) was dissolved in a 4 M solution of HCl in 1,4-dioxane (2.0 mL) and stirred at room temperature for 30 min. The volatiles were evaporated to afford the title compound (90 mg, yield: 92%) as a beige solid. The product was used in the next step without further purification. LC-MS (Method A7) m/z: [M+H]⁺: 281/283; rt: 2.22 min; purity: 91%.

### Step 7: Synthesis of 14-chloro-4,12-dimethyl-5,6,9,13-tetrazatricyclo[8.4.0.0^{2,6}]tetradeca-1(14),2,4,10,12-pentaen-8-one N30

13-Chloro-7-(chloromethyl)-4,11-dimethyl-5,6,8,12-tetrazatricyclo[7.4.0.0^{2,6}]trideca-1(13),2,4,7,9,11-hexaene hydrochloride (intermediate N30_6, 90.0 mg, 0.258 mmol) was dissolved in 1,4-dioxane (5 mL) and water (2 mL) and aqueous NaOH (2 M, 0.26 mL, 0.516 mmol) was added. The reaction mixture was stirred at room temperature for 1.5 h, then diluted with water (10 mL) and acidified to pH ~3-5 with aqueous HCl (1 M, 1 mL). The aqueous layer was extracted with EtOAc (2×20 mL) and the combined organic extracts were filtered through a hydrophobic frit and concentrated under vacuum. The residue was triturated with MTBE (5 mL) to afford the title compound (38 mg, yield: 53%) as a beige solid. The product was used in the next step without further purification. LC-MS (Method B5′) m/z: [M+H]⁺: 263/265; rt: 1.15 min; purity: 98%. ¹H NMR (400 MHz, DMSO-d₆) δ 10.89 (s, 1H), 7.05 (s, 1H), 6.63 (s, 1H), 4.83 (s, 2H), 2.47 (s, 3H), 2.23 (s, 3H).

### Intermediate N31: 3-chloro-5,10-dimethyl-4,8,12-triazatricyclo[9.4.0.0^{2,7}]pentadeca-1(11),2,4,6,12,14-hexaen-9-one

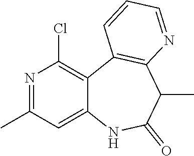

### Step 1: Synthesis of ethyl 2-[3-(4,4,5,5-tetramethyl-1,3,2-dioxaborolan-2-yl)-2-pyridyl]propanoate N31_1

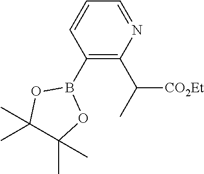 [SMILES: CCOC(=O)C(C)c1ncccc1B1OC(C)(C)C(C)(C)O1]

Ethyl 2-(3-bromo-2-pyridyl)propanoate (Intermediate N68_2, 30.0 g, 116 mmol), bis(pinacolato)diboron (35.4 g, 139 mmol) and potassium acetate (45.6 g, 465 mmol) were suspended in dry 1,4-dioxane (500 mL) and the reaction mixture was degassed with nitrogen for 10 min. Pd(dppf)Cl₂ (5.95 g, 8.14 mmol) was added and the reaction mixture was stirred at 80° C. for 24 h. The reaction mixture was cooled to room temperature and filtered through a small pad of Celite®. The filter cake was rinsed with EtOAc (200 mL) and the filtrate was concentrated under vacuum. The residue was purified by flash chromatography on silica gel (using a gradient of 0 to 100% MTBE in iso-hexane as eluent) to afford the title compound (32.3 g, yield: 45%) as a yellow oil. ¹H NMR (400 MHz, CDCl₃) δ 8.60 (dd, J=4.8, 2.0 Hz, 1H), 8.10 (d, J=7.4 Hz, 1H), 7.17 (d, J=6.6 Hz, 1H), 4.71 (d, J=7.1 Hz, 1H), 4.14 (q, J=7.2 Hz, 2H), 1.55-1.51 (m, 3H), 1.34 (s, 12H), 1.18 (d, J=6.9 Hz, 3H).

### Step 2: Synthesis of 3-chloro-5,10-dimethyl-4,8,12-triazatricyclo[9.4.0.0^{2,7}]pentadeca-1(11),2,4,6,12,14-hexaen-9-one N31

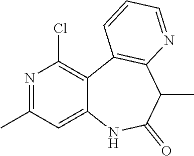

A mixture of 3-bromo-2-chloro-6-methyl-pyridin-4-amine (Intermediate N30_1, 400 mg, 1.81 mmol), ethyl 2-[3-(4,4,5,5-tetramethyl-1,3,2-dioxaborolan-2-yl)-2-pyridyl]propanoate (Intermediate N31_1, 1.10 g, 2.53 mmol), cesium fluoride (768 mg, 5.06 mmol) and bis(di-tert-butyl(4-dimethylaminophenyl)phosphine)dichloropalladium(II) (64 mg, 0.090 mmol) in 1,4-dioxane (34 mL) and water (1.7 mL), was purged with nitrogen then heated at reflux for 18 h. The reaction was allowed to cool to room temperature, then filtered through a bed of Celite® and washed with EtOAc (100 mL). The filtrate was concentrated under vacuum then taken up in absolute EtOH (16 mL) to which potassium carbonate (499 mg, 3.61 mmol) was added and the reaction mixture was heated at 65° C. for 4 h. The reaction mixture was concentrated under vacuum, then water (75 mL) was added and the reaction mixture was extracted with EtOAc (3×75 mL). The combined organic layers were dried, filtered and concentrated under vacuum. The residue was purified by flash chromatography on silica gel (using a gradient of 0 to 100% EtOAc in iso-hexane as eluent) to afford the title compound (178 mg, yield: 35%) as an off-white solid. LC-MS (Method A7) m/z: [M+H]⁺: 274; rt: 1.45 min; purity: 99%. ¹H NMR (400 MHz, DMSO-d₆) δ 10.69 (s, 1H), 8.64 (dd, J=4.7, 1.7 Hz, 1H), 8.18 (dd, J=7.9, 1.7 Hz, 1H), 7.44 (dd, J=7.9, 4.7 Hz, 1H), 7.05 (s, 1H), 3.64 (q, J=6.6 Hz, 1H), 2.48 (s, 3H), 1.47 (d, J=6.6 Hz, 3H).

The racemate was separated by chiral chromatography (SFC Chiralpak AS from Daicel, CO₂+isopropanol 20%). Chiral purity 98.6%; rt=1.79 min. (second eluting enantiomer, N31_A). For information, first eluting enantiomer N31_B rt=1.615 min. Both measured by HPLC, Chiralpak AS from Daicel, Solvent: Heptane 50%—Ethanol 50%—DEA 0.1%.

### Intermediate N32: 1-chloro-7-methoxy-3-methyl-5,7-dihydropyrido[3,4-a][3]benzazepin-6-one

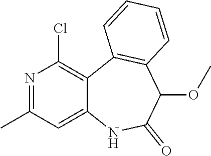

### Step 1: Synthesis of methyl 2-bromo-2-(2-bromophenyl)acetate N32_1

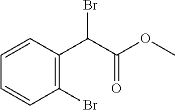

A mixture of methyl 2-(2-bromophenyl)acetate (4.71 g, 20.6 mmol), 2,2′-azobis(2-methylpropionitrile) (338 mg, 2.06 mmol) and N-bromosuccinimide (3.66 g, 20.6 mmol) in chloroform (50 mL) was heated under reflux for 4 h. The reaction mixture was cooled to room temperature, washed with brine (2×50 mL) and the organic phase was dried over sodium sulfate, filtered and concentrated under vacuum to afford the title compound (6.20 g, yield: 88%) as a colourless oil. ¹H NMR (400 MHz, CDCl₃) δ 7.80 (dd, J=7.9, 1.7 Hz, 1H), 7.61-7.58 (m, 1H), 7.39 (td, J=7.6, 1.3 Hz, 1H), 7.25-7.20 (m, 1H), 5.93 (s, 1H), 3.83 (s, 3H).

### Step 2: Synthesis of methyl 2-(2-bromophenyl)-2-methoxy-acetate N32_2

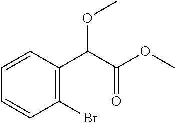

To a solution of sodium methoxide prepared from sodium metal (542 mg, 23.6 mmol) and methanol (90 mL) was added methyl 2-bromo-2-(2-bromophenyl)acetate (Intermediate N32_1, 6.20 g, 18.1 mmol) and the reaction mixture was heated at reflux for 30 min. The reaction mixture was cooled to room temperature and the solvent was removed under vacuum. The residue was taken up into EtOAc (30 mL), washed with brine (20 mL), the organic phases were separated, dried over sodium sulfate, filtered and concentrated under vacuum. The product was purified by flash chromatography on silica gel (using a gradient of 0 to 10% EtOAc in iso-hexane as eluent) to afford the title compound (2.10 g, yield: 44%) as a colourless oil. ¹H NMR (400 MHz, CDCl₃) δ 7.64-7.57 (m, 1H), 7.51 (dt, J=8.3, 2.4 Hz, 1H), 7.41-7.33 (m, 1H), 7.28-7.19 (m, 1H), 5.30 (d, J=2.5 Hz, 1H), 3.76 (d, J=1.5 Hz, 3H), 3.45 (d, J=2.3 Hz, 3H).

### Step 3: Synthesis of methyl 2-methoxy-2-[2-(4,4,5,5-tetramethyl-1,3,2-dioxaborolan-2-yl)phenyl]acetate N32_3

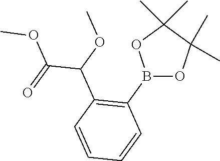

To a suspension of methyl 2-(2-bromophenyl)-2-methoxy-acetate (Intermediate N32_2, 1.00 g, 3.78 mmol), bis(pinacolato)diboron (1.15 g, 4.54 mmol) and potassium acetate (1.48 g, 15.1 mmol) in dry 1,4-dioxane (40 mL) under inert atmosphere was added [1,1′-bis(diphenylphosphino)ferrocene]dichloropalladium(II) (138 mg, 0.189 mmol) and the suspension was heated at reflux for 8 h. The reaction mixture was filtered through a pad of Celite® which was washed with EtOAc (50 mL). The filtrate was concentrated under vacuum and the residue was purified by flash chromatography on silica gel (using a gradient of 0 to 30% MTBE in iso-hexane as eluent) to afford the title compound (270 mg, yield: 18%). LC-MS (Method B5′) m/z: [M+H]⁺: 307; rt: 2.03 min; purity: 80%.

### Step 4: Synthesis of methyl 2-[2-(4-amino-2-chloro-6-methyl-3-pyridyl)phenyl]-2-methoxy-acetate N32_4

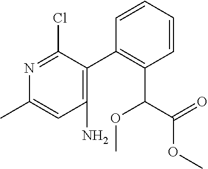

A mixture of 3-bromo-2-chloro-6-methyl-pyridin-4-amine (Intermediate N30_1, 350 mg, 1.50 mmol), methyl 2-methoxy-2-[2-(4,4,5,5-tetramethyl-1,3,2-dioxaborolan-2-yl)phenyl]acetate (Intermediate N32_3, 469 mg, 1.50 mmol) and cesium fluoride (684 mg, 4.50 mmol) in dry 1,4-dioxane (30.0 mL) was degassed for 10 min. SPhos Pd(crotyl)Cl (Pd-172, CAS: 1798781-99-3, 91 mg, 0.15 mmol) was added and the reaction mixture was heated at 90° C. for 8 hours. The reaction mixture was allowed to cool to room temperature and poured onto water (100 mL). The crude product was extracted with EtOAc (2×50 mL) and the organic layers were combined and dried over sodium sulfate. The crude product was purified by flash chromatography on silica gel (using a gradient of 0 to 100% EtOAc in iso-hexane as eluent) to give a mixture of the title compound (123 mg, yield: 5%) and 1-chloro-7-methoxy-3-methyl-5,7-dihydropyrido[4,3-d][3]benzazepin-6-one (123 mg, yield: 22%). The product was used in the next step without further purification. LC-MS (Method A7) m/z: [M+H]⁺: 321/323; rt: 1.01 min; purity: 20% and m/z: [M+H]⁺: 281/291; rt: 1.66 min; purity: 80%.

### Step 5: Synthesis of 1-chloro-7-methoxy-3-methyl-5,7-dihydropyrido[3,4-a][3]benzazepin-6-one N32

A mixture of methyl 2-[2-(4-amino-2-chloro-6-methyl-3-pyridyl)phenyl]-2-methoxy-acetate (Intermediate N32_4, 125 mg, 0.078 mmol) and potassium carbonate (32 mg, 0.234 mmol) in ethanol (5.0 mL) was heated under reflux for 2 h. The reaction mixture was evaporated to dryness and to the residue was added water (10 mL). The reaction mixture was adjusted to pH=5 by the addition of a few drops of glacial acetic acid. The resulting precipitate was collected by filtration, washed with water (5 mL) and dried under vacuum to afford the title compound (89 mg, yield: 97%) as a white solid. LC-MS (Method A7) m/z: [M+H]⁺: 289/291; rt: 1.66 min; purity: 98%.

### Intermediate N33: 3,5-dimethoxy-4,8,12-triazatricyclo[9.4.0.0^{2,7}]pentadeca-1(11),2(7),3,5,12,14-hexaen-9-one

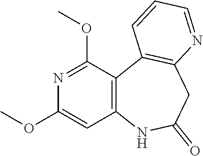 [SMILES: COc1cc2c(c(OC)n1)-c1cccnc1CC(=O)N2]

### Step 1: Synthesis of 3-bromo-2,6-dimethoxy-pyridin-4-amine N33_1

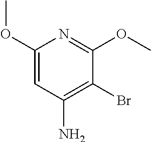

To a solution of 2,6-dimethoxypyridin-4-amine (300 mg, 1.85 mmol) in DCM (18 mL) at 0° C. was added N-bromosuccinimide (329 mg, 1.85 mmol). The reaction mixture was stirred at 0° C. for 1 h then at room temperature overnight. Water (20 mL) was then added and the phases were separated. The aqueous layer was extracted with DCM (2×20 mL). The organic layers were combined, washed with brine (50 mL), dried over Na₂SO₄, filtered and concentrated under vacuum. The residue was purified by flash chromatography on silica gel (using a gradient of 0 to 100% EtOAc in iso-hexane as eluent) to give the title compound (360 mg, yield: 83%) as a white solid. LC-MS (Method A7) m/z: [M+H]⁺: 233/235; rt: 1.78 min; purity: 99%. ¹H NMR (400 MHz, DMSO-d₆) δ 6.10 (s, 2H), 5.75 (s, 1H), 3.82 (s, 3H), 3.75 (s, 3H).

### Step 2: Synthesis of ethyl 2-[3-(4-amino-2,6-dimethoxy-3-pyridyl)-2-pyridyl]acetate N33_2

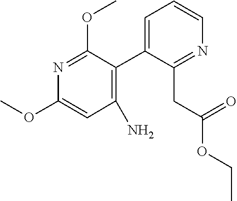 [SMILES: CCOC(=O)Cc1ncccc1-c1c(N)cc(OC)nc1OC]

Nitrogen was passed through a suspension of 3-bromo-2,6-dimethoxy-pyridin-4-amine (Intermediate N33_1, 145 mg, 0.616 mmol), ethyl 2-[3-(4,4,5,5-tetramethyl-1,3,2-dioxaborolan-2-yl)-2-pyridyl]acetate (Intermediate N25_2, 280 mg, 0.863 mmol), SPhos (CAS 657408-07-6 51 mg, 0.012 mmol) and cesium fluoride (294 mg, 1.85 mmol) in 1,4-dioxane (5.3 mL) and water (0.3 mL) for 5 min. Then palladium(II) acetate (11 mg, 0.048 mmol) was added. The reaction mixture was heated at reflux overnight then cooled to room temperature and filtered through a pad of Celite® and washed with EtOAc (60 mL). The filtrate was concentrated under vacuum. The residue was purified by flash chromatography on silica gel (using a gradient of 0 to 10% MeOH in DCM as eluent) to give the title compound (332 mg, yield: 61%) as a brown oil. LC-MS (Method A7) m/z: [M+H]⁺: 318.2; rt: 1.36 min; purity: 36%.

### Step 3: Synthesis of 3,5-dimethoxy-4,8,12-triazatricyclo[9.4.0.0^{2,7}]pentadeca-1(11),2(7),3,5,12,14-hexaen-9-one N33

A suspension of ethyl 2-[3-(4-amino-2,6-dimethoxy-3-pyridyl)-2-pyridyl]acetate (Intermediate N33_2, 332 mg, 0.377 mmol), 3 A molecular sieves (1.00 g) and anhydrous potassium carbonate (156 mg, 1.13 mmol) in absolute EtOH (5.0 mL) was heated at reflux overnight. The reaction mixture was cooled to room temperature, filtered through a pad of Celite® then washed with a 1:1 mixture of DCM/MeOH (75 mL). The filtrate was concentrated under vacuum and the residue was purified by flash chromatography on silica gel (using a gradient of 0 to 8% MeOH in DCM as eluent), followed by trituration in MTBE (10 mL) to give the title compound (60 mg, yield: 59%) as an off-white solid. LC-MS (Method A7) m/z: [M+H]⁺: 272; rt: 1.38 min; purity: >99%. ¹H NMR (400 MHz, DMSO-d₆) δ 10.39 (s, 1H), 8.48 (dd, J=4.8, 1.7 Hz, 1H), 8.11 (dd, J=8.0, 1.7 Hz, 1H), 7.39 (dd, J=8.0, 4.8 Hz, 1H), 6.24 (s, 1H), 3.93 (s, 3H), 3.92 (s, 3H), 3.69 (d, J=12.4 Hz, 1H), 3.57 (d, J=12.4 Hz, 1H).

### Intermediate N34: 3-fluoro-5-methyl-4,8,12,13-tetrazatricyclo[9.4.0.0^{2,7}]pentadeca-1(11),2,4,6,12,14-hexaen-9-one

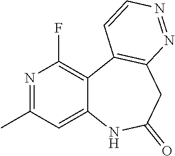 [SMILES: Cc1cc2c(c(F)n1)-c1ccnnc1CC(=O)N2]

### Step 1: Synthesis of 3,4-dichloropyridazine N34_1

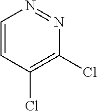

To a solution of 5-chloro-1H-pyridazin-6-one (3.0 g, 23.0 mmol) in dry acetonitrile (25 mL), was added phosphorus oxychloride (5.3 mL, 57.5 mmol) at room temperature and the reaction mixture was heated at 80° C. overnight. The reaction mixture was allowed to cool to room temperature, then poured onto ice/water. The reaction mixture was diluted with saturated aqueous NaHCO₃ (50 mL) then extracted with DCM (2×50 mL). The organic layers were combined, dried over Na₂SO₄, filtered and concentrated under vacuum to give the title compound (3.39 g, yield: 92%) as a brown solid that was used in the next step without further purification. LCMS (Method A7) m/z: [M+H]⁺: 149; rt: 1.04 min; Purity 98%.

### Step 2: Synthesis of 4-(benzyloxy)-3-chloropyridazine N34_2

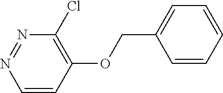 [SMILES: Clc1nnccc1OCc1ccccc1]

At 0° C., a solution of benzyl alcohol (1.49 g, 13.7 mmol) in dry THF (10 mL) was added dropwise to a suspension of NaH (60.0% dispersion in oil, 599 mg, 15.0 mmol) in dry THF (20 mL). The reaction mixture was stirred at 0° C. for 30 min, then allowed to warm to room temperature. A solution of 3,4-dichloropyridazine (Intermediate N34_1, 2.0 g, 12.5 mmol) in THF (10 mL) was added dropwise and the reaction mixture was stirred at room temperature for 2 hours. The reaction mixture was treated with water and extracted with EtOAc (2×30 mL). The combined organic extracts were dried over Na₂SO₄, filtered and concentrated under vacuum. The residue was then purified by flash chromatography on silica gel (using a gradient of 0 to 10% MeOH in DCM as eluent) to afford the title compound (2.21 g, yield: 77%) as a brown oil. LCMS (Method A7) m/z: [M+H]⁺: 221; rt: 1.77 min; Purity: 97%.

### Step 3: Synthesis of ethyl 2-(4-(benzyloxy)pyridazin-3-yl)acetate N34_3

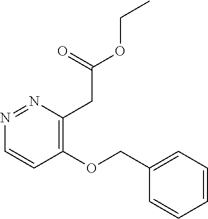 [SMILES: CCOC(=O)Cc1nnccc1OCc1ccccc1]

A degassed solution of 4-benzyloxy-3-chloro-pyridazine (Intermediate N34_2, 200 mg, 0.880 mmol), bromo-(2-ethoxy-2-oxo-ethyl)zinc (0.48 M solution in THF, 3.7 mL, 1.76 mmol), tris(dibenzylideneacetone)dipalladium(0) (25 mg, 0.044 mmol) and X-Phos (CAS 564483-18-7, 19 mg, 0.044 mmol) in dry THF (5.0 mL) was stirred at 65° C. for 4 h. The reaction mixture was concentrated under vacuum, then treated by saturated aqueous NH₄Cl (20 mL) and extracted with DCM (2×25 mL). The organic layers were combined, dried over Na₂SO₄, filtered and then concentrated under vacuum. The residue was purified by flash chromatography on silica gel (using a gradient of 0 to 10% MeOH in DCM as eluent) to give the title compound as a yellow oil. LCMS (Method A7) m/z: [M+H]⁺: 273; rt: 1.59 min; purity: 77%.

### Step 4: Synthesis of ethyl 2-(4-hydroxypyridazin-3-yl)acetate N34_4

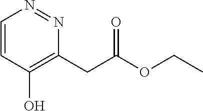

To a solution of ethyl 2-(4-benzyloxypyridazin-3-yl)acetate (Intermediate N34_3, 195 mg, 0.573 mmol) in ethanol (8 mL) was added 10% Pd/C (31 mg, 0.028 mmol) and the reaction mixture was stirred under hydrogen atmosphere (P=5 bars) for 3 h. The reaction mixture was filtered through Celite® and the filtrate was concentrated under vacuum to give the title compound (135 mg, yield: 75%) as an orange solid. The product was used in the next step with no further purification. LCMS (Method A7) m/z: [M+H]⁺: 183 rt: 0.57 min; purity: 58%.

### Step 5: Synthesis of ethyl 2-(4-chloropyridazin-3-yl)acetate N34_5

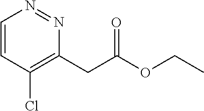

To a solution of ethyl 2-(4-hydroxypyridazin-3-yl)acetate (Intermediate N34_4, 200 mg, 0.56 mmol) in dry acetonitrile (4 mL), was added phosphorus oxychloride (0.13 mL, 1.41 mmol) at room temperature and the reaction mixture was heated at 80° C. for 1 hour. The reaction mixture was allowed to cool to room temperature, then poured onto ice/water. Saturated aqueous NaHCO₃ (15 mL) was added then the aqueous phase was extracted with DCM (2×15 mL). The organic layers were combined, dried over Na₂SO₄, filtered and concentrated under vacuum. The residue was purified by flash chromatography on silica gel (using a gradient of 0 to 100% EtOAc in iso-hexane as eluent) to afford the title compound (33 mg, yield: 28%) as a yellow solid. LC-MS (Method A7) m/z: [M+H]⁺: 201/203; rt: 1.23 min; purity: 97%.

### Step 6: Synthesis of 3-fluoro-5-methyl-4,8,12,13-tetrazatricyclo[9.4.0.0^{2,7}]pentadeca-1(11),2,4,6,12,14-hexaen-9-one N34

A mixture of ethyl 2-(4-chloropyridazin-3-yl)acetate (Intermediate N34_5, 44 mg, 0.196 mmol), 2-fluoro-6-methyl-3-(4,4,5,5-tetramethyl-1,3,2-dioxaborolan-2-yl)pyridin-4-amine (Intermediate N16_1, 137 mg, 0.49 mmol), SPhos Pd(crotyl)Cl (CAS: 1798781-99-3, 12 mg, 0.020 mmol), aqueous potassium carbonate (1.50 M, 0.4 mL, 0.59 mmol) and cesium fluoride (89 mg, 0.588 mmol) in 1,4-dioxane (6 mL) was stirred at 90° C. overnight under inert atmosphere. The reaction mixture was cooled to room temperature and filtered through a small pad of Celite. The filter cake was rinsed with EtOAc (10 mL) and the filtrate was concentrated under vacuum. The residue was purified by flash chromatography on silica gel (using a gradient of 0 to 10% MeOH in DCM as eluent) to afford the title compound (19 mg, yield: 35%) as an off-white solid. LC-MS (Method A7) m/z: [M+H]⁺: 245; rt: 0.88 min; purity: 88%. ¹H NMR (400 MHz, CD₃₀D) δ 9.28 (d, J=5.4 Hz, 1H), 8.07 (dd, J=5.4, 4.3 Hz, 1H), 7.05 (s, 1H), 4.31-3.97 (m, 2H), 2.55 (s, 3H). NH proton was not observed.

### Intermediate N35: 14-fluoro-7,12-dimethyl-5,6,9,13-tetrazatricyclo[8.4.0.0^{2,6}]tetradeca-1(10),2,4,11,13-pentaen-8-one

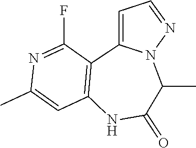

### Step 1: Synthesis of 2-fluoro-6-methyl-3-(2-tetrahydropyran-2-ylpyrazol-3-yl)pyridin-4-amine N35_1

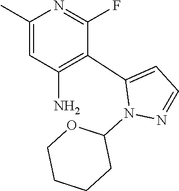 [SMILES: Cc1cc(N)c(-c2ccnn2C2CCCCO2)c(F)n1]

To a solution of 4-amino-3-bromo-2-fluoro-6-methylpyridine (400 mg, 1.95 mmol) and 1-(2-tetrahydropyranyl)-1H-pyrazole-5-boronic acid pinacol ester (657 mg, 2.34 mmol) in dry toluene (10 mL) was added K₃PO₄ (1.28 g, 5.85 mmol). The white suspension was degassed with Argon for 5 min, then tris(dibenzylideneacetone)dipalladium(0) (184 mg, 0.19 mmol) and 2-dicyclohexylphosphino-2,6-dimethoxybiphenyl (163 mg, 0.39 mmol) were added and the reaction mixture was stirred at 100° C. for 16 h in a sealed reactor. After cooling to room temperature, the reaction mixture was diluted with EtOAc, filtered through a pad of celite, rinsed with EtOAc, and the filtrate was concentrated under vacuum. The residue was triturated with Et₂O and collected by filtration to give the title product (455 mg, yield: 45%) as an orange solid. LC-MS (Method-B1) m/z: [M+H]⁺: 277.0, rt: 0.98 min, purity: 53%.

### Step 2: Synthesis of 14-fluoro-7,12-dimethyl-5,6,9,13-tetrazatricyclo[8.4.0.0^{2,6}]tetradeca-1(10),2,4,11,13-pentaen-8-one N35

To a solution of 2-fluoro-6-methyl-5-(2-tetrahydropyran-2-ylpyrazol-3-yl)pyridin-4-amine (Intermediate N35_1, 100 mg, 0.36 mmol) in dry DMF (5 mL) were added dropwise 2-bromopropionyl chloride (107 μL, 0.87 mmol) and diisopropylethylamine (180 μL, 1.1 mmol) and the reaction mixture was stirred at room temperature for 3 h. 2-Bromopropionyl chloride (107 μL, 0.87 mmol) and diisopropylethylamine (180 μL, 1.1 mmol) were added again and the reaction mixture was stirred at 130° C. for 1 h. After cooling to room temperature, EtOAc was added and the organic phase was washed three times with water. The organic layer was dried on MgSO₄, filtered off and concentrated under vacuum. Purification by preparative TLC on silica gel (using 5% EtOH in dichloromethane as eluent) afforded the title product (24 mg, yield: 21%) as an orange oil. LC-MS (Method-B1) m/z: [M+H]⁺: 247.05, rt: 0.94 min, purity: 80%.

### Intermediate N36: 15-fluoro-8,13-dimethyl-4,6,10,14-tetrazatricyclo[9.4.0.0^{2,7}]pentadeca-1(15),2(7),3,5,11,13-hexaen-9-one

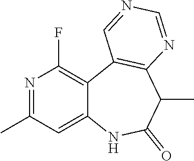

### Step 1: Synthesis of 1-(tert-butyl) 3-ethyl 2-(5-bromopyrimidin-4-yl)malonate N36_1

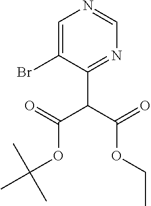 [SMILES: CCOC(=O)C(C(=O)OC(C)(C)C)c1ncncc1Br]

To a suspension of NaH (60% dispersion in oil, 12.4 g, 310 mmol) in dry DMF (400 mL) at 0° C. was added tert-butyl ethyl malonate (53.5 g, 284 mmol) in dry DMF (50 mL) over 45 min and the reaction mixture was stirred for a further 45 min at 0° C. A solution of 5-bromo-4-chloro-pyrimidine (50.0 g, 258 mmol) in dry DMF (50 mL) was added and the reaction mixture was stirred for 30 min at 0° C. then allowed to reach room temperature and stirred for a further 20 h. The reaction mixture was diluted with saturated aqueous ammonium chloride (1 L) and extracted with EtOAc (3×350 mL). The organic layers were combined, washed with brine (3×500 mL), dried over sodium sulfate, filtered and concentrated under vacuum. The residue was purified by flash chromatography on silica gel (using a gradient of 0 to 50% EtOAc in iso-hexane as eluent) to give the title compound (38.9 g, yield: 41%) as a yellow oil. LC-MS (Method A7) m/z: [M-C₄H₈+H]⁺: 289/291; rt: 2.16 min; purity: >99%. ¹H NMR (400 MHz, DMSO-d₆) δ 9.17 (s, 1H), 9.08 (s, 1H), 5.14 (s, 1H), 4.30-4.14 (m, 2H), 1.43 (s, 9H), 1.21 (t, J=7.1 Hz, 3H).

### Step 2: Synthesis of ethyl 2-(5-bromopyrimidin-4-yl)acetate N36_2

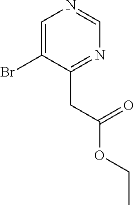

To a solution of 1-(tert-butyl) 3-ethyl 2-(5-bromopyrimidin-4-yl)malonate (Intermediate N36_1, 38.9 g, 107 mmol) in DCM (300 mL) cooled with an ice bath, was added TFA (100 mL) and the reaction mixture was warmed to room temperature and stirred for 18 h. The volatiles were removed under vacuum and azeotroped with toluene (100 mL). The residue was purified by flash chromatography on silica gel (using a gradient of 0 to 50% EtOAc in iso-hexane as eluent) to give the title compound (29.5 g, quantitative yield) as a yellow oil. LC-MS (Method A7) m/z: [M+H]⁺: 245/247; rt: 1.42 min; purity: 90%. ¹H NMR (400 MHz, DMSO-d₆) δ 9.11 (s, 1H), 9.02 (s, 1H), 4.13 (q, J=7.1 Hz, 2H), 4.02 (s, 2H), 1.19 (t, J=7.1 Hz, 3H).

### Step 3: Synthesis of ethyl 2-(5-bromopyrimidin-4-yl)propanoate N36_3

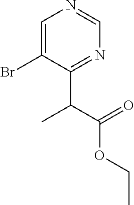

To a solution of ethyl 2-(5-bromopyrimidin-4-yl)acetate (Intermediate N36_2, 29.8 g, 108 mmol) in dry THF (400 mL) cooled with an ice bath, was added over 20 min lithium bis(trimethylsilyl)amide (1 M solution in THF, 130 mL, 130 mmol) and the reaction mixture was stirred at 0° C. for 1 h. Iodomethane (23.0 g, 162 mmol) was added dropwise and the reaction mixture was stirred at 0° C. for 1 h then at room temperature for a further 16 h. The reaction mixture was diluted with saturated aqueous ammonium chloride (600 mL) and extracted with EtOAc (3×400 mL). The organic layers were combined, washed with saturated aqueous ammonium chloride (500 mL), brine (300 mL), dried over sodium sulfate, filtered and concentrated under vacuum. The residue was purified by flash chromatography on silica gel (using a gradient of 0 to 50% EtOAc in iso-hexane as eluent) to give the title compound (12.6 g, yield: 43%) as a yellow oil. LC-MS (Method A7) m/z: [M+H]*: 259/261; rt: 1.7 min; purity>99%. ¹H NMR (400 MHz, DMSO-d₆) δ 9.12 (s, 1H), 9.01 (s, 1H), 4.29 (q, J=7.1 Hz, 1H), 4.09 (q, J=7.1 Hz, 2H), 1.44 (d, J=7.1 Hz, 3H), 1.12 (t, J=7.1 Hz, 3H).

### Step 4: Synthesis of 15-fluoro-8,13-dimethyl-4,6,10,14-tetrazatricyclo[9.4.0.0^{2,7}]pentadeca-1(15),2(7),3,5,11,13-hexaen-9-one N36

A stirring mixture of ethyl 2-(5-bromopyrimidin-4-yl)propanoate (Intermediate N36_3, 90 mg, 0.347 mmol), 2-fluoro-6-methyl-3-(4,4,5,5-tetramethyl-1,3,2-dioxaborolan-2-yl)pyridin-4-amine (Intermediate N16_1, 138 mg, 0.521 mmol) and aqueous potassium carbonate (1.5 M, 0.7 mL, 1.04 mmol) in 1,4-dioxane (5 mL) was degassed with nitrogen for 5 min. Bis(di-tert-butyl(4-dimethylaminophenyl)phosphine)-dichloropalladium(II) (25 mg, 0.035 mmol) was added and the reaction mixture was degassed with nitrogen for further 5 min. The reaction mixture was heated at 90° C. for 3 hours and then cooled to room temperature. The reaction mixture was diluted with EtOAc (20 mL) and filtered through a small pad of Celite. The filter cake was rinsed with EtOAc (10 mL) and the filtrate was concentrated under vacuum. The residue was dissolved in EtOH (5 mL) and potassium carbonate (96 mg, 0.695 mmol) was added and the reaction mixture was stirred at 80° C. for 2 hours. The reaction mixture was cooled to room temperature and filtered through a small pad of Celite. The filter cake was rinsed with EtOH (10 mL) and the filtrate was concentrated under vacuum. The residue was purified by flash chromatography on silica gel (using a gradient of 0 to 10% MeOH in DCM as eluent) to afford the title compound (28 mg, yield: 30%) as a pink solid. LC-MS (Method A7) m/z: [M+H]⁺: 259; rt: 1.25 min; purity: 98%. ¹H NMR (400 MHz, DMSO-d₆) δ 11.02 (s, 1H), 9.24 (s, 1H), 9.09 (d, J=4.7 Hz, 1H), 7.03 (s, 1H), 3.74 (d, J=6.6 Hz, 1H), 2.47 (s, 3H), 1.49 (d, J=6.6 Hz, 3H).

### Intermediate N37: 5-fluoro-3-methoxy-4,8,12-triazatricyclo[9.4.0.0^{2,7}]pentadeca-1(11),2(7),3,5,12,14-hexaen-9-one

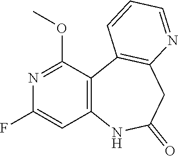 [SMILES: COc1nc(F)cc2c1-c1cccnc1CC(=O)N2]

### Step 1: Synthesis of 2-fluoro-6-methoxy-pyridin-4-amine N37_1

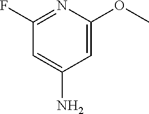

A solution of 2,6-difluoropyridin-4-amine (760 mg, 5.84 mmol) and sodium methoxide (5.4 M solution in methanol, 2.4 mL, 12.9 mmol) in dry THF (29 mL) was heated at reflux for 4 h. The reaction mixture was diluted with water (30 mL) then extracted with EtOAc (3×30 mL). The combined extracts were washed with brine (60 mL), dried over Na₂SO₄, filtered and concentrated under vacuum. The residue was purified by flash chromatography on silica gel (using a gradient of 0 to 60% EtOAc in iso-hexane as eluent) to give the title compound (725 mg, yield: 78%) as a yellow oil. LC-MS (Method A7) m/z: [M+H]⁺: 143; rt: 0.96 min; purity: 55%. ¹H NMR (400 MHz, CDCl₃) δ 5.82 (dd, J=1.6, 1.0 Hz, 1H), 5.78 (d, J=1.6 Hz, 1H), 4.25 (s, 2H), 3.87 (s, 3H).

### Step 2: Synthesis of 3-bromo-6-fluoro-2-methoxy-pyridin-4-amine N37_2

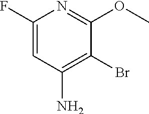

To a solution of 2-fluoro-6-methoxy-pyridin-4-amine (Intermediate N37_1, 160 mg, 1.01 mmol) in DCM (10.0 mL) was added a suspension of N-bromosuccinimide (198 mg, 1.11 mmol) in DCM (10 mL). The reaction mixture was stirred at room temperature for 3 h. A second portion of N-bromosuccinimide (20 mg, 0.11 mmol) was added and the reaction mixture was stirred overnight at room temperature. The reaction mixture was then heated at reflux overnight. A third portion of N-bromosuccinimide (40 mg, 0.22 mmol) was added and the reaction mixture was stirred at room temperature for 4 h. Water (10 mL) was added and the phases were separated. The aqueous layer was extracted with DCM (2×10 mL). The combined extracts were washed with brine (40 mL), dried over Na₂SO₄, filtered and concentrated under vacuum. The residue was purified by flash chromatography on silica gel (using a gradient of 0 to 50% EtOAc in iso-hexane as eluent) to give the title compound (203 mg, yield: 91%) as a white solid. LC-MS (Method A7) m/z: [M+H]⁺: 221/223; rt: 1.69 min; purity>99%. ¹H NMR (400 MHz, DMSO-d₆) δ 6.59 (s, 2H), 5.99 (s, 1H), 3.80 (s, 3H).

### Step 3: Synthesis of ethyl 2-[3-(4-amino-6-fluoro-2-methoxy-3-pyridyl)-2-pyridyl]acetate N37 3

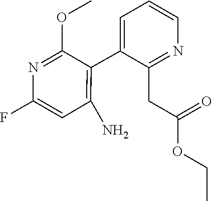 [SMILES: CCOC(=O)Cc1ncccc1-c1c(N)cc(F)nc1OC]

To a suspension of 3-bromo-6-fluoro-2-methoxy-pyridin-4-amine (Intermediate N37_2, 108 mg, 0.488 mmol), ethyl 2-[3-(4,4,5,5-tetramethyl-1,3,2-dioxaborolan-2-yl)-2-pyridyl]acetate (Intermediate N25_2, 222 mg, 0.684 mmol), SPhos (40 mg, 0.098 mmol) and cesium fluoride (223 mg, 1.44 mmol) in 1,4-dioxane (5.2 mL) and water (0.3 mL) under inert atmosphere was added palladium(II) acetate (11 mg, 0.049 mmol). The reaction mixture was heated at reflux overnight then cooled to room temperature, filtered through a pad of Celite® and washed with EtOAc (50 mL). The filtrate was concentrated under vacuum and the residue was purified by flash chromatography on silica gel (using a gradient of 0 to 6% MeOH in DCM as eluent) to give the title compound (259 mg, quantitative yield) as a brown oil. LC-MS (Method A7) m/z: [M+H]⁺: 306; rt: 1.35 min; purity: 60%.

### Step 4: Synthesis of 5-fluoro-3-methoxy-4,8,12-triazatricyclo[9.4.0.0^{2,7}]pentadeca-1(11),2(7),3,5,12,14-hexaen-9-one N37

A suspension of ethyl 2-[3-(4-amino-6-fluoro-2-methoxy-3-pyridyl)-2-pyridyl]acetate (Intermediate N37_3, 259 mg, 0.488 mmol), potassium carbonate (300 mg, 2.17 mmol) and 3 A molecular sieves (1.50 g) in EtOH (5.0 mL) was heated at reflux overnight. The reaction mixture was cooled to room temperature, filtered through a pad of Celite® and washed with a 2:1 solution of DCM/MeOH (50 mL). The filtrate was concentrated under vacuum and the resulting residue was purified by flash chromatography on silica gel (using a gradient of 0 to 8% MeOH in DCM as eluent) and trituration in MTBE (10 mL) to give the title compound (35 mg, yield: 25%) as an off-white solid. LC-MS (Method A7) m/z: [M+H]⁺: 260; rt: 1.34 min; purity: 89%. ¹H NMR (400 MHz, DMSO-d₆) δ 10.68 (s, 1H), 8.54 (dd, J=4.7, 1.7 Hz, 1H), 8.17 (dd, J=8.0, 1.7 Hz, 1H), 7.43 (dd, J=8.0, 4.7 Hz, 1H), 6.53 (d, J=1.2 Hz, 1H), 3.91 (s, 3H), 3.76 (d, J=12.5 Hz, 1H), 3.63 (d, J=12.5 Hz, 1H).

### Intermediate N39: 1-chloro-7-(methoxymethyl)-3-methyl-5,7-dihydropyrido[4,3-d][3]benzazepin-6-one

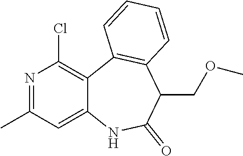

### Step 1: Synthesis of methyl 2-(2-bromophenyl)-3-methoxy-propanoate N39_1

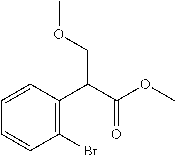

At −60° C., to a stirred solution of methyl 2-(2-bromophenyl)acetate (7.20 g, 31.4 mmol) in dry THE (100 mL) was added lithium diisopropylamide (2 M solution in THF/ethyl benzene/heptane, 17 mL, 34.6 mmol) over 20 min maintaining the temperature at −60° C. Upon completion of addition, the reaction mixture was stirred at this temperature for further 20 min before dropwise addition of chloromethyl methyl ether (3.1 mL, 34.6 mmol). The reaction mixture was warmed to room temperature, then treated with water (200 mL) and extracted with EtOAc (2×100 mL). The organic extracts were combined, dried over sodium sulfate, filtered and concentrated under vacuum. The residue was purified by flash chromatography on silica gel (using a gradient of 0 to 10% MTBE in iso-hexane as eluent) to afford the title compound (6.0 g, yield: 69%) as a colorless oil. ¹H NMR (500 MHz, CDCl₃) δ 7.61 (dd, J=8.0, 1.3 Hz, 1H), 7.43 (dd, J=7.8, 1.7 Hz, 1H), 7.31 (td, J=7.6, 1.3 Hz, 1H), 7.16 (ddd, J=8.0, 7.4, 1.7 Hz, 1H), 4.50 (dd, J=8.6, 5.2 Hz, 1H), 3.95-3.91 (m, 1H), 3.74 (s, 3H), 3.65 (dd, J=9.5, 5.2 Hz, 1H), 3.41 (s, 3H).

### Step 2: Synthesis of methyl 3-methoxy-2-[2-(4,4,5,5-tetramethyl-1,3,2-dioxaborolan-2-yl)phenyl]propanoate N39_2

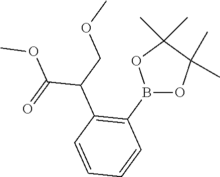

To a suspension of methyl 2-(2-bromophenyl)-3-methoxy-propanoate (Intermediate N39_1, 1.66 g, 5.96 mmol), in 1,4-dioxane (30 mL) under nitrogen atmosphere was added [1,1′-[1,1′-bis(diphenylphosphino)ferrocene]dichloropalladium(II) (218 mg, 0.298 mmol), bis(pinacolato)diboron (1.81 g, 7.15 mmol) and potassium acetate (2.34 mg, 23.8 mmol). The suspension was heated at 90° C. for 18 hours. After cooling to room temperature, the reaction mixture was filtered through a pad of Celite® which was washed with EtOAc (50 mL). The filtrate was concentrated under vacuum and the residue was purified by flash chromatography on silica gel (using a gradient of 0 to 50% MTBE in iso-hexane as eluent) to afford the title compound (1.32 g, yield: 55%) as a colorless oil. ¹H NMR (400 MHz, CDCl₃) δ 7.87-7.83 (m, 1H), 7.41 (dd, J=6.5, 1.7 Hz, 2H), 7.28 (td, J=6.9, 6.3, 2.2 Hz, 1H), 4.97 (dd, J=9.2, 4.8 Hz, 1H), 4.01 (t, J=9.4 Hz, 1H), 3.70 (s, 3H), 3.57 (dd, J=9.5, 4.9 Hz, 1H), 3.40 (s, 3H), 1.38 (d, J=3.3 Hz, 12H).

### Step 3: Synthesis of methyl 2-[2-(4-amino-2-chloro-6-methyl-3-pyridyl)phenyl]-3-methoxy-propanoate N39_3

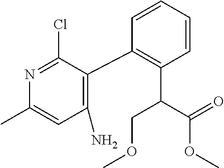

A mixture of 3-bromo-2-chloro-6-methyl-pyridin-4-amine (Intermediate N30_1, 25 mg, 0.112 mmol), methyl 3-methoxy-2-[2-(4,4,5,5-tetramethyl-1,3,2-dioxaborolan-2-yl)phenyl]propanoate (Intermediate N39_2, 30 mg, 0.094 mmol), aqueous potassium carbonate (1.5 M, 0.08 mL, 0.120 mmol) and bis(di-tert-butyl(4-dimethylaminophenyl)phosphine)dichloropalladium(II) (7 mg, 0.009 mmol) in dry 1,4-dioxane (2.0 mL) was heated at 90° C. for 4 hours. The reaction mixture was cooled to room temperature, diluted with water (5 mL) and extracted with DCM (5 mL). The organic extracts were separated, dried over MgSO₄, filtered and concentrated under vacuum. The residue was purified by flash chromatography on silica gel (using a gradient of 0 to 100% EtOAc in iso-hexane as eluent) to afford the title compound (22 mg, yield: 69%) as a pale-yellow gum. LC-MS (Method A7) m/z: [M+H]⁺: 335/337; rt: 1.21 and 1.78 min; purity: 98%.

### Step 4: Synthesis of 1-chloro-7-(methoxymethyl)-3-methyl-5,7-dihydropyrido[4,3-d][3]benzazepin-6-one N39

To a solution of methyl 2-[2-(4-amino-2-chloro-6-methyl-3-pyridyl)phenyl]-3-methoxy-propanoate (Intermediate N39_3, 200 mg, 0.597 mmol) in dry THF (10.0 mL) was added dropwise at room temperature a 1 M solution of lithium bis(trimethylsilyl)amide in MTBE (0.66 mL, 0.66 mmol) and the reaction mixture was stirred for 1 hour. An additional aliquot of lithium bis(trimethylsilyl)amide in MTBE (0.33 mL, 0.330 mmol) was added and the reaction mixture was stirred for a further 1 hour. The reaction mixture was cooled with an ice/water bath, treated with water (10 mL) and extracted with EtOAc (2×10 mL). The combined organic extracts were washed with water (10 mL), dried over sodium sulfate, filtered and concentrated under vacuum. Trituration in cold MTBE (2 mL) afforded the title compound (122 mg, yield: 66%) as pale-yellow solid. LC-MS (Method A7) m/z: [M+H]⁺: 303/305; rt: 1.74 min; purity: 98%. ¹H NMR (400 MHz, DMSO-d₆) δ 10.60 (s, 1H), 7.74 (dd, J=7.9, 1.4 Hz, 1H), 7.49 (td, J=7.6, 1.4 Hz, 1H), 7.39 (td, J=7.6, 1.3 Hz, 1H), 7.28 (d, J=7.8 Hz, 1H), 7.02 (s, 1H), 4.08 (d, J=7.1 Hz, 2H), 3.49 (s, 1H), 3.31 (s, 3H), 2.48 (s, 3H).

### Intermediate N40: 3-fluoro-5-methoxy-4,8,12-triazatricyclo[9.4.0.0^{2,7}]pentadeca-1(11),2(7),3,5,12,14-hexaen-9-one

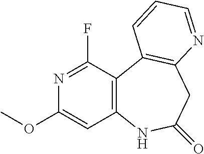 [SMILES: COc1cc2c(c(F)n1)-c1cccnc1CC(=O)N2]

### Step 1: Synthesis of 2-fluoro-6-methoxy-3-(4,4,5,5-tetramethyl-1,3,2-dioxaborolan-2-yl)pyridin-4-amine N40_1

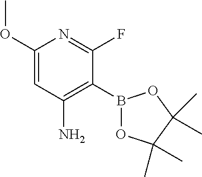 [SMILES: COc1cc(N)c(B2OC(C)(C)C(C)(C)O2)c(F)n1]

A solution of 2-fluoro-6-methoxy-pyridin-4-amine (Intermediate N37_1, 211 mg, 1.34 mmol) in dry THE (6.4 mL) was degassed with nitrogen for 5 min then 4,4,5,5-tetramethyl-1,3,2-dioxaborolane (0.20 mL, 1.43 mmol) was added dropwise. The resulting mixture was stirred at room temperature for 30 min then bis(pinacolato)diboron (CAS 73183-34-3, 376 mg, 1.48 mmol), 4-tert-butyl-2-(4-tert-butyl-2-pyridyl)pyridine (36 mg, 0.134 mmol) and (1,5-cyclooctadiene)(methoxy)iridium(I) dimer (45 mg, 0.07 mmol) were added. The resulting mixture was degassed with nitrogen for 5 min then stirred at 80° C. overnight. The reaction mixture was filtered through a pad of Celite, washed with EtOAc (10 mL) and the filtrate was concentrated under vacuum. The residue was purified by flash chromatography on silica gel (using a gradient of 0 to 100% EtOAc in iso-hexane as eluent) to give the title compound (265 mg, yield: 59%) as a white solid. LC-MS (Method A7) m/z: [M+H]⁺: 269; rt: 2.02 min; purity: 70%. ¹H NMR (400 MHz, DMSO-d₆) δ 6.57 (s, 2H), 6.29 (t, J=1.3 Hz, 1H), 2.16 (s, 3H), 1.28 (s, 12H).

### Step 2: Synthesis of ethyl 2-[3-(4-amino-2-fluoro-6-methoxy-3-pyridyl)-2-pyridyl]acetate N40_2

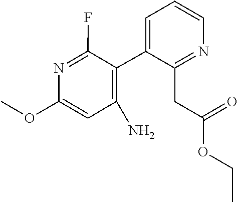 [SMILES: CCOC(=O)Cc1ncccc1-c1c(N)cc(OC)nc1F]

Nitrogen was passed through a suspension of ethyl 2-(3-bromo-2-pyridyl)acetate (Intermediate N3_1, 82 mg, 0.336 mmol), 2-fluoro-6-methoxy-3-(4,4,5,5-tetramethyl-1,3,2-dioxaborolan-2-yl)pyridin-4-amine (Intermediate N40_1, 141 mg, 0.420 mmol) and cesium fluoride (179 mg, 1.18 mmol) in dry toluene (1.2 mL), EtOH (0.6 mL) and water (0.6 mL) for 5 min. Then PEPPSI™_IPent (Dichloro[1,3-bis(2,6-di-3-pentylphenyl)imidazol-2-ylidene](3-chloropyridyl)palladium(II) (CAS 1158652-41-5, 27 mg, 0.034 mmol) was added. The reaction mixture was heated at 80° C. overnight then cooled to room temperature, filtered through a pad of Celite, washed with EtOAc (50 mL) and the filtrate was concentrated under vacuum. The residue was purified by flash chromatography on silica gel (using a gradient of 0 to 7% MeOH in DCM as eluent) to give the title compound (158 mg, quantitative yield) as a yellow oil. LC-MS (Method A7) m/z: [M+H]⁺: 306; rt: 1.39 min; purity: 80%.

### Step 3: Synthesis of 3-fluoro-5-methoxy-4,8,12-triazatricyclo[9.4.0.0²⁷]pentadeca-1(11),2(7),3,5,12,14-hexaen-9-one N40

A suspension of ethyl 2-[3-(4-amino-2-fluoro-6-methoxy-3-pyridyl)-2-pyridyl]acetate (Intermediate N40_2, 158 mg, 0.34 mmol), potassium carbonate (207 mg, 1.50 mmol) and 3 A molecular sieves (1.0 g) in absolute EtOH (5.0 mL) was heated at reflux overnight. After cooling to room temperature, the reaction mixture was filtered through a pad of Celite® and washed with a 2:1 solution of DCM/MeOH (50 mL). The filtrate was concentrated under vacuum and the residue was purified by flash chromatography on silica gel (using a gradient of 0 to 8% MeOH/DCM as eluent) and trituration in MTBE (10 mL) to give the title compound (33 mg, yield: 30%) as a white solid. LC-MS (Method A7) m/z: [M+H]⁺: 260; rt: 1.37 min; purity: 88%. ¹H NMR (400 MHz, DMSO-d₆) δ 10.72 (s, 1H), 8.57 (dd, J=4.8, 1.6 Hz, 1H), 8.07 (ddd, J=8.0, 4.8, 1.6 Hz, 1H), 7.47 (dd, J=8.0, 4.8 Hz, 1H), 6.56 (s, 1H), 3.90 (s, 3H), 3.87 (d, J=12.6 Hz, 1H), 3.62 (d, J=12.6 Hz, 1H).

### Intermediate N40A: 3-fluoro-5-methoxy-4,8,12-triazatricyclo[9.4.0.0^{2,7}]pentadeca-1(11),2(7),3,5,12,14-hexaen-9-one

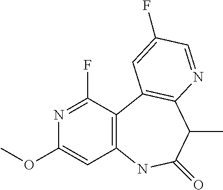

The title compound was prepared according to the same reaction sequence as the one described for intermediate N40 starting from intermediate N40_1 and methyl 2-(3-bromo-5-fluoro-2-pyridyl)propanoate. Methyl 2-(3-bromo-5-fluoro-2-pyridyl)propanoate was prepared by methylation of methyl 2-(3-bromo-5-fluoro-2-pyridyl)acetate (CAS: 1804408-40-9) according to the same procedure as described for example NN213. The first step (Suzuki reaction) was performed with Pd₂dba₃, SPhos and K₃PO₄ in toluene at 100° C. The second step was performed using LiHMDS in THF at RT, both steps similar as those described for intermediate NN142.

### Intermediate N40B: 3-fluoro-5-methoxy-4,8,12-triazatricyclo[9.4.0.0^{2,7}]pentadeca-1(11),2(7),3,5,12,14-hexaen-9-one

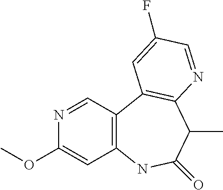

The title compound was prepared according to the same reaction sequence as the one described for intermediate N40A starting from intermediate NN232 and methyl 2-(3-bromo-5-fluoro-2-pyridyl)propanoate. Methyl 2-(3-bromo-5-fluoro-2-pyridyl)propanoate was prepared by methylation of methyl 2-(3-bromo-5-fluoro-2-pyridyl)acetate (CAS: 1804408-40-9) according to the same procedure as described or example NN213. The first step (Suzuki reaction) was performed with Pd₂dba₃, SPhos and K₃PO₄ in toluene at 100° C. The second step was performed using LiHMDS in THF at RT, both steps similar as those described for intermediate NN142.

### Intermediate N41: 4-ethyl-5-methyl-4,8,12-triazatricyclo[9.4.0.0^{2,7}]pentadeca-1(11),2(7),5,12,14-pentaene-3,9-dione

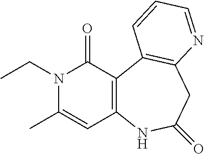 [SMILES: CCn1c(C)cc2c(c1=O)-c1cccnc1CC(=O)N2]

### Step 1: Synthesis of 4-(benzylamino)-1-ethyl-6-methylpyridine-2(1H)-one N41_1

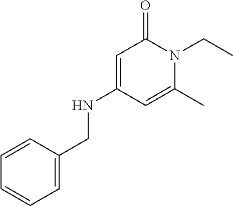 [SMILES: CCn1c(C)cc(NCc2ccccc2)cc1=O]

A solution of 1-ethyl-4-hydroxy-6-methyl-pyridin-2-one (250 mg, 1.63 mmol) in benzylamine (1.75 g, 16.3 mmol) was heated at 170° C. for 18 hours. The resulting pale brown precipitate was collected by filtration, washed with MTBE then dried under vacuum to afford the title compound (102 mg, yield: 25%) as an off white solid. LC-MS (Method A7) m/z [M+H]⁺: 243; rt: 1.53 min; purity: 99%. ¹H NMR (400 MHz, DMSO-d₆) δ 7.31 (m, 4H), 7.26-7.21 (m, 1H), 6.90 (t, J=6.0 Hz, 1H), 5.63 (d, J=2.5 Hz, 1H), 5.02 (d, J=2.5 Hz, 1H), 4.20 (d, J=5.9 Hz, 2H), 3.79 (q, J=7.0 Hz, 2H), 2.22 (s, 3H), 1.05 (t, J=7.0 Hz, 3H).

### Step 2: Synthesis of 4-amino-1-ethyl-6-methylpyridine-2(1H)-one N41_2

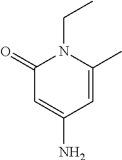

To a solution of 4-(benzylamino)-1-ethyl-6-methyl-pyridin-2-one (Intermediate N41_1, 100 mg, 0.413 mmol) in glacial acetic acid (10.0 mL) was added 10% Pd/C (439 mg, 0.413 mmol). The reaction mixture was purged with nitrogen followed by hydrogen gas (3×) then stirred under hydrogen atmosphere (P=2 bars) at room temperature for 18 h. The reaction mixture was filtered through a bed of Celite®, washed with toluene (5 mL) and the filtrate was evaporated. This was further co-evaporated with toluene (3×5 mL) to give a gummy pale brown solid which was triturated with MTBE (5 mL). The resulting grey solid was collected by filtration then purified using a 2 g SCX (pre-conditioned) column, eluting with neat methanol followed by 0.7 M ammonia-methanol to afford the title compound (50 mg, yield: 79%) as an off white waxy solid. LC-MS (Method A7) m/z [M+H]⁺: 153; rt: 0.61 min; purity: 99%. ¹H NMR (400 MHz, DMSO-d₆) δ 5.78 (s, 2H), 5.51-5.49 (m, 1H), 5.14 (d, J=2.4 Hz, 1H), 3.81 (q, J=7.0 Hz, 2H), 2.21 (s, 3H), 1.07 (t, J=7.0 Hz, 3H).

### Step 3: Synthesis of 4-amino-3-bromo-1-ethyl-6-methyl-pyridin-2-one N41_3

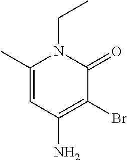

To a suspension of 4-amino-1-ethyl-6-methylpyridine-2(1H)-one (Intermediate N41_2, 42 mg, 0.273 mmol) in dry acetonitrile (1.0 mL) was added N-bromosuccinimide (49 mg, 0.273 mmol) and the reaction mixture was stirred for 21 hours at room temperature. The volatiles were removed under vacuum and the residue was purified by flash chromatography on silica gel (using a gradient of 0 to 10% of (1% NH₃) MeOH in DCM) to afford the title compound (42 mg, yield: 59%) as an off white solid. LC-MS (Method A7) m/z [M+H]⁺: 231/233; rt: 0.97 min; purity>99%. ¹H NMR (400 MHz, DMSO-de) δ 6.13 (s, 2H), 5.72 (d, J=1.0 Hz, 1H), 3.89 (q, J=7.0 Hz, 2H), 2.24 (d, J=0.8 Hz, 3H), 1.10 (t, J=7.0 Hz, 3H).

### Step 4: Synthesis of ethyl 2-[3-(4-amino-1-ethyl-6-methyl-2-oxo-3-pyridyl)-2-pyridyl]acetate N41_4

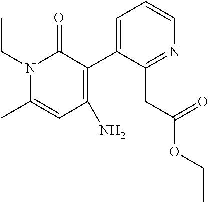 [SMILES: CCOC(=O)Cc1ncccc1-c1c(N)cc(C)n(CC)c1=O]

A solution of 4-amino-3-bromo-1-ethyl-6-methyl-pyridin-2-one (Intermediate N41_3, 42 mg, 0.162 mmol), ethyl 2-[3-(4,4,5,5-tetramethyl-1,3,2-dioxaborolan-2-yl)-2-pyridyl]acetate (Intermediate N25_2, 94 mg, 0.324 mmol), bis(di-tert-butyl(4-dimethylaminophenyl)phosphine)dichloropalladium (II) (12 mg, 0.016 mmol) and aqueous potassium carbonate (1.5 M, 0.11 mL, 0.162 mmol) in 1,4-dioxane (5 mL) was heated at 80° C. for 18 h under inert atmosphere. Then, the reaction mixture was heated at 90° C. for 18 hours. The reaction mixture was cooled to room temperature then diluted with EtOAc (20 mL). The resulting suspension was filtered through a small pad of Celite* and the filter cake was rinsed with EtOAc (10 mL). The filtrate was concentrated under vacuum and purified by flash chromatography on silica gel (using a gradient of 0 to 10% (NH₃/MeOH) in DCM as eluent) to afford the title compound (2 mg, yield: 1%) as a brown oil. LC-MS (Method A7) m/z [M+H]⁺: 316; rt: 0.81 min; purity: 35%.

### Step 5: Synthesis of 4-ethyl-5-methyl-4,8,12-triazatricyclo[9.4.0.0² 7]pentadeca-1(11),2(7),5,12,14-pentaene-3,9-dione N41

To a solution of ethyl 2-[3-(4-amino-1-ethyl-6-methyl-2-oxo-3-pyridyl)-2-pyridyl]acetate (Intermediate N41_4, 2 mg, 0.006 mmol) in ethanol (1.00 mL) was added potassium carbonate (2 mg, 0.013 mmol) and the reaction mixture was heated at reflux for 18 hours. The volatiles were removed under vacuum and co-evaporated with toluene (2 mL). The resulting solid was taken to next step without purification or analysis.

### Intermediate N42: 3-chloro-5,10-dimethyl-4,8,10,12-tetrazatricyclo[9.4.0.0^{2,7}]pentadeca-1(11),2,4,6,12,14-hexaen-9-one

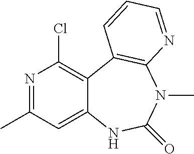

### Step 1: Synthesis of N-methyl-3-(4,4,5,5-tetramethyl-1,3,2-dioxaborolan-2-yl)pyridin-2-amine N42_1

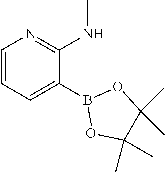 [SMILES: CNc1ncccc1B1OC(C)(C)C(C)(C)O1]

To a solution of 3-bromo-N-methyl-pyridin-2-amine (1.50 g, 8.02 mmol) in dry 1,4-dioxane (20.0 mL) was added bis(pinacolato)diboron (CAS 73183-34-3, 2.44 g, 9.62 mmol), [1,1′-bis (diphenylphosphino)ferrocene]dichloropalladium(II) (293 mg, 0.401 mmol) and potassium acetate (3.15 g, 32.1 mmol). The reaction mixture was heated at reflux for 16 h. The reaction mixture was cooled to room temperature and filtered through Celite. The filtrate was concentrated under vacuum to afford the title compound as a brown oil which was used in the next step with no further purification. LC-MS (Method B5′) m/z: [M+H]⁺: 235; rt: 0.44 min; purity: 60%.

### Step 2: Synthesis of 2-chloro-6-methyl-3-[2-(methylamino)-3-pyridyl]pyridin-4-amine N42_2

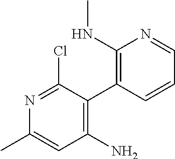 [SMILES: CNc1ncccc1-c1c(N)cc(C)nc1Cl]

To a solution of N-methyl-3-(4,4,5,5-tetramethyl-1,3,2-dioxaborolan-2-yl)pyridin-2-amine (Intermediate N42_1, 634 mg, 2.71 mmol) in 1,4-dioxane (8.0 mL) and water (2.0 mL) was added 3-bromo-2-chloro-6-methyl-pyridin-4-amine (Intermediate N30_1, 500 mg, 2.26 mmol), bis(di-tert-butyl(4-dimethylaminophenyl)phosphine)dichloropalladium (II) (160 mg, 0.226 mmol) and K₂CO₃ (936 mg, 6.77 mmol) and the reaction mixture was heated at reflux for 2 h. After cooling to room temperature, the reaction mixture was filtered through Celite® and concentrated under vacuum. Purification by column chromatography on silica gel (using a gradient of 0 to 5% MeOH in DCM as eluent) afforded the title compound (220 mg, yield: 75%) as a yellow solid. LC-MS (Method A7) m/z: [M+H]⁺: 249/251; rt: 1.24 min; purity: 75%.

### Step 3: Synthesis of 3-chloro-5,10-dimethyl-4,8,10,12-tetrazatricyclo[9.4.0.0^{2,7}]pentadeca-1(11),2,4,6,12,14-hexaen-9-one N42

To a solution of 2-chloro-6-methyl-3-[2-(methylamino)-3-pyridyl]pyridin-4-amine (Intermediate N42_2, 220 mg, 0.885 mmol) in dry DMF (5.0 mL) was added 1,1′-carbonyldiimidazole (158 mg, 0.973 mmol) and triethylamine (0.22 mL, 1.59 mmol) at room temperature and the reaction mixture was heated at reflux for 16 h. After cooling to room temperature, the reaction mixture was treated with water (10.0 mL) and extracted with EtOAc (3×20 mL). The combined organic extracts were washed with brine (2×20 mL), dried over MgSO₄, filtered and concentrated under vacuum. Purification by column chromatography on silica gel (using a gradient of 0 to 2% MeOH in DCM as eluent) afforded the title compound (150 mg, yield: 46%) as a yellow solid. LC-MS (Method A7) m/z: [M+H]⁺: 275/277; rt: 1.56 min; purity: 75%.

### Intermediate N43: 15-methoxy-8,13-dimethyl-4,6,10,14-tetrazatricyclo[9.4.0.0^{2,7}]pentadeca-1(15),2(7),3,5,11,13-hexaen-9-one

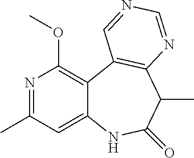

### Step 1: Synthesis of 2-methoxy-6-methyl-3-(4,4,5,5-tetramethyl-1,3,2-dioxaborolan-2-yl)pyridin-4-amine N43_1

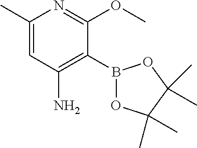

To a mixture of 3-bromo-2-methoxy-6-methylpyridin-4-amine (Intermediate N64_3, 2.33 g, 9.83 mmol), bis(pinacolato)diboron (7.49 g, 29.5 mmol), PCy3 Pd G₂ (Chloro[(tricyclohexylphosphine)-2-(2′-aminobiphenyl)]palladium(II), CAS 1353658-81-7, 581 mg, 0.983 mmol) and potassium acetate (3.86 g, 39.3 mmol) was added 1,4-dioxane (20.0 mL) and the reaction mixture was stirred at 90° C. for 20 h. After cooling to room temperature, the reaction mixture was filtered through Celite® eluting with EtOAc (100 mL), then the filtrate was concentrated under vacuum. The residue was purified by flash chromatography on silica gel (using a gradient of 0 to 100% EtOAc in iso-hexane as eluent) to afford the title product which was used directly in the next step without further purification or analysis.

### Step 2: Synthesis of 15-methoxy-8,13-dimethyl-4,6,10,14-tetrazatricyclo[9.4.0.0^{2,7}]pentadeca-1(15),2(7),3,5,11,13-hexaen-9-one N43

A suspension of ethyl 2-(5-bromopyrimidin-4-yl)propanoate (Intermediate N36_3, 1.20 g, 4.40 mmol), 2-methoxy-6-methyl-3-(4,4,5,5-tetramethyl-1,3,2-dioxaborolan-2-yl)pyridin-4-amine (Intermediate N43_2, 1.11 g, 0.127 mmol), SPhos Pd(crotyl)Cl (CAS: 1798781-99-3, 41 mg, 0.067 mmol) and cesium fluoride (1.82 g, 12.0 mmol) in dry 1,4-dioxane (15 mL) was heated at 90° C. for 4 h. The reaction mixture was cooled to room temperature, then filtered through Celite® eluting with EtOAc (100 mL) and concentrated under vacuum. The residue was dissolved in ethanol (15 mL) and potassium carbonate (1.32 g, 9.54 mmol) was added. The reaction mixture was stirred at 80° C. for 18 h. The volatiles were removed under vacuum and the residue was purified by flash chromatography on silica gel (using a gradient of 0 to 10% MeOH in DCM as eluent) to give the title compound (464 mg, yield: 5%). LC-MS (Method A7) m/z: [M+H]⁺: 271; rt: 1.48 min; purity: 20%.

### Intermediate N44: 1-chloro-10-methoxy-3,7-dimethyl-5,7-dihydropyrido[4,3-d][3]benzazepin-6-one

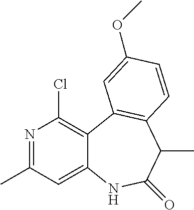

### Step 1: Synthesis of ethyl 2-[4-methoxy-2-(4,4,5,5-tetramethyl-1,3,2-dioxaborolan-2-yl)phenyl]propanoate N44_1

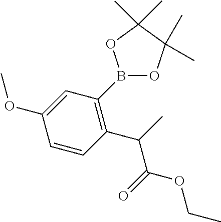 [SMILES: CCOC(=O)C(C)c1ccc(OC)cc1B1OC(C)(C)C(C)(C)O1]

To a suspension of ethyl 2-(2-bromo-4-methoxy-phenyl)propanoate (511 mg, 1.78 mmol), bis(pinacolato)diboron (542 mg, 2.14 mmol), potassium acetate (699 mg, 7.12 mmol) and 3A molecular sieves (0.5 g) in dry 1,4-dioxane (10 mL) under inert atmosphere was added [1,1′-bis(diphenylphosphino)ferrocene]dichloropalladium(II) (65 mg, 0.089 mmol) and the reaction mixture was heated at 100° C. for 18 h. After cooling to room temperature, the reaction mixture was filtered through a small pad of Celite. The filter cake was rinsed with EtOAc (20 mL) and the filtrate was concentrated under vacuum to afford the title compound (910 mg, yield: 99%) which was used in the next step without further purification or analysis.

### Step 2: Synthesis of ethyl 2-[2-(4-amino-2-chloro-6-methyl-3-pyridyl)-4-methoxy-phenyl]propanoate N44_2

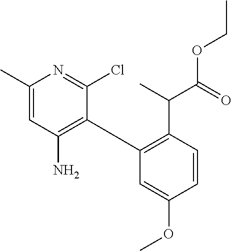 [SMILES: CCOC(=O)C(C)c1ccc(OC)cc1-c1c(N)cc(C)nc1Cl]

To a solution of 3-bromo-2-chloro-6-methyl-pyridin-4-amine (Intermediate N30_1, 100 mg, 0.429 mmol) in 1,4-dioxane (10 mL) were added ethyl 2-[4-methoxy-2-(4,4,5,5-tetramethyl-1,3,2-dioxaborolan-2-yl)phenyl]propanoate (Intermediate N44_1, 307 mg, 0.643 mmol), bis(di-tert-butyl(4-dimethylaminophenyl)phosphine)dichloropalladium(II) (30 mg, 0.043 mmol) and an aqueous solution of K₂CO₃ (1.5 M, 2.0 mL, 3.00 mmol). The reaction mixture was purged with nitrogen for 5 min, then heated at 90° C. for 3 hours. The reaction mixture was cooled to room temperature and filtered through a small pad of Celite® and the filtrate was concentrated under vacuum. Saturated aqueous NH₄Cl (5 mL) was added and the mixture was extracted with DCM (3×10 mL). The combined organic layers were washed with brine (5 mL), dried over Na₂SO₄, filtered and concentrated under vacuum. The residue was purified by flash chromatography on silica gel (using a gradient of 0 to 100% EtOAc in iso-hexane as eluent) to give the title compound (234 mg, quantitative yield) as a mixture of two diastereoisomers which was used directly in the next step without further purification. LC-MS (Method A7) m/z [M+H]⁺: 349/351; rt: 1.89/1.98 min; purity 65%.

### Step 3: Synthesis of 1-chloro-10-methoxy-3,7-dimethyl-5,7-dihydropyrido[4,3-d][3]benzazepin-6-one N44

At 0° C., to a solution of ethyl 2-[2-(4-amino-2-chloro-6-methyl-3-pyridyl)-4-methoxy-phenyl]propanoate (Intermediate N44_2, 150 mg, 0.430 mmol) in dry toluene (4 mL), was added a 1 M solution of lithium bis(trimethylsilyl)amide in THF (1.30 mL, 1.29 mmol) and the reaction mixture was stirred at room temperature for 1 h. The reaction mixture was neutralized by addition of saturated aqueous NH₄Cl and extracted with EtOAc (3×20 mL). The combined organic layers were washed with brine, dried over Na₂SO₄, filtered and concentrated under vacuum to afford the title compound (66 mg, yield: 45%). LC-MS (Method A7) m/z: [M+H]⁺: 303/305; rt: 1.79 min; purity: 90%.

### Intermediate N45: 1-fluoro-8-(hydroxymethyl)-3-methyl-5,7-dihydropyrido[3,4-a][3]benzazepin-6-one

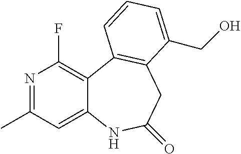 [SMILES: Cc1cc2c(c(F)n1)-c1cccc(CO)c1CC(=O)N2]

### Step 1: Synthesis of ethyl 3-bromo-2-methylbenzoate N45_1

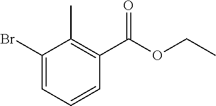

To a stirred solution of 3-bromo-2-methylbenzoic acid (2.0 g, 9.30 mmol) in EtOH (15.0 mL) was added concentrated H₂SO₄ (547 mg, 5.58 mmol) and the resulting solution was stirred at 90° C. for 18 h. The volatiles were evaporated under reduced pressure and the residue was dissolved in EtOAc (40 mL) and washed with saturated aqueous NaHCO₃ (2×50 mL). The organic phase was dried over anhydrous Na₂SO₄, filtered and concentrated under vacuum to afford the title compound (2.1 g, yield: 91%) as a pale orange oil. LC-MS (Method A7) m/z: [M+H]P: 243/245; rt: 2.50 min; purity: 98%. ¹H NMR (400 MHz, CDCl₃) δ 7.96-7.58 (m, 2H), 7.19-6.91 (m, 1H), 4.37 (q, J=7.1 Hz, 2H), 2.63 (s, 3H), 1.39 (t, J=7.1 Hz, 3H).

### Step 2: Synthesis of ethyl 3-bromo-2-(bromomethyl)benzoate N45_2

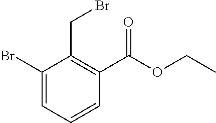

To a stirred solution of ethyl 3-bromo-2-methylbenzoate (Intermediate N45_1, 1.0 g, 4.11 mmol) in chloroform (15.0 mL) was added N-bromosuccinimide (879 mg, 4.94 mmol) and benzoyl peroxide (50 mg, 0.206 mmol) and the reaction mixture was heated at reflux for 16 h. After cooling to room temperature, the reaction mixture was filtered off and the filtrate was collected, diluted with chloroform (15 mL) then washed with aqueous NaOH (2 M, 40 mL), water (30 mL) and brine (30 mL). The organic layer was dried over anhydrous Na₂SO₄, filtered and concentrated under vacuum to afford the title compound (900 mg, yield: 61%) as a brown oil. ¹H NMR (400 MHz, CDCl₃) δ 7.87 (dd, J=7.8, 1.3 Hz, 1H), 7.75 (dd, J=8.0, 1.4 Hz, 1H), 7.24 (d, J=12.6 Hz, 1H), 5.12 (s, 2H), 4.42 (q, J=7.1 Hz, 2H), 1.43 (t, J=7.1 Hz, 3H).

### Step 3: Synthesis of ethyl 3-bromo-2-(cyanomethyl)benzoate N45_3

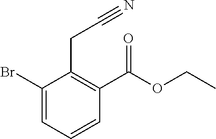

To a stirred solution of ethyl 3-bromo-2-(bromomethyl)benzoate (Intermediate N45_2, 900 mg, 2.52 mmol) in DMSO (10.0 mL) was added sodium cyanide (185 mg, 3.77 mmol) and the resulting solution was stirred at room temperature for 18 h. The reaction mixture was diluted with water (90 mL) and extracted with EtOAc (2×20 mL). The organic layers were combined, dried over anhydrous Na₂SO₄, filtered and concentrated under vacuum to afford the crude title compound (790 mg, yield: 98%) as an off white solid. ¹H NMR (400 MHz, CDCl₃) δ 7.99 (dd, J=7.9, 1.4 Hz, 1H), 7.81 (dd, J=8.0, 1.4 Hz, 1H), 7.30 (t, J=7.9 Hz, 1H), 4.43 (q, J=7.1 Hz, 2H), 4.37 (s, 2H), 1.43 (t, J=7.1 Hz, 3H).

### Step 4: Synthesis of ethyl 3-bromo-2-(2-ethoxy-2-oxoethyl)benzoate N45_4

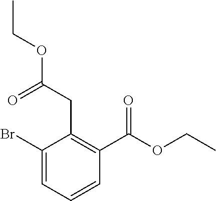 [SMILES: CCOC(=O)Cc1c(Br)cccc1C(=O)OCC]

A solution of ethyl 3-bromo-2-(cyanomethyl)benzoate (Intermediate N45_3, 790 mg, 2.48 mmol) in ethanol (6.0 mL) and a 4 M solution of HCl in 1,4-dioxane (6.0 mL) was heated at 90° C. for 16 h. The volatiles were removed under vacuum and the residue was dissolved in EtOAc (30 mL). The organic phase was washed with saturated aqueous NaHCO₃ solution (2×40 mL), dried over anhydrous Na₂SO₄, filtered and concentrated under vacuum. The residue was purified by flash chromatography on silica gel (using a gradient of 0 to 100% EtOAc in iso-hexane as eluent) to give the title compound (270 mg, yield: 33%) as a colorless oil. LC-MS (Method B5′) m/z: [M+H]⁺: 315/317; rt: 2.36 min; purity: 96%. ¹H NMR (400 MHz, CDCl₃) δ 7.92 (dd, J=7.8, 1.4 Hz, 1H), 7.76 (dd, J=8.0, 1.4 Hz, 1H), 7.21 (t, J=7.9 Hz, 1H), 4.34 (q, J=7.2 Hz, 2H), 4.29 (s, 2H), 4.18 (q, J=7.1 Hz, 2H), 1.37 (t, J=7.1 Hz, 3H), 1.26 (t, J=7.1 Hz, 3H).

### Step 5: Synthesis of ethyl 3-(4-amino-2-fluoro-6-methyl-3-pyridyl)-2-(2-ethoxy-2-oxo-ethyl)benzoate N45_5

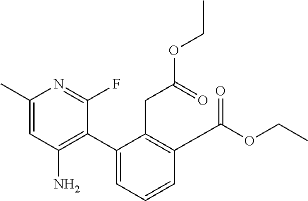 [SMILES: CCOC(=O)Cc1c(C(=O)OCC)cccc1-c1c(N)cc(C)nc1F]

To a stirred solution of ethyl 3-bromo-2-(2-ethoxy-2-oxo-ethyl)benzoate (Intermediate N45_4, 160 mg, 0.487 mmol) in 1,4-dioxane (7.0 mL) and water (0.5 mL) was added 2-fluoro-6-methyl-3-(4,4,5,5-tetramethyl-1,3,2-dioxaborolan-2-yl)pyridin-4-amine (Intermediate N16_1, 164 mg, 0.585 mmol), SPhos Pd(crotyl)Cl (CAS: 1798781-99-3, 30 mg, 0.049 mmol) and cesium fluoride (222 mg, 1.46 mmol) and the reaction mixture was heated at 90° C. overnight. After cooling to room temperature, the reaction mixture was concentrated under vacuum and directly purified by flash chromatography on silica gel (using a gradient of 0 to 100% EtOAc in iso-hexane as eluent) to give the title compound (210 mg, yield: 92%) as pale brown oil. LC-MS (Method B5′) m/z: [M+H]⁺: 361; rt: 1.93 min; purity: 77%.

### Step 6: Synthesis of ethyl 1-fluoro-3-methyl-6-oxo-5,7-dihydropyrido[4,3-d][3]benzazepine-8-carboxylate N45_6

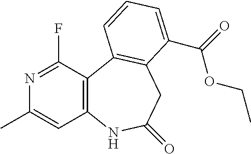 [SMILES: CCOC(=O)c1cccc2c1CC(=O)Nc1cc(C)nc(F)c1-2]

To a stirred solution of ethyl 3-(4-amino-2-fluoro-6-methyl-3-pyridyl)-2-(2-ethoxy-2-oxo-ethyl)benzoate (Intermediate N45_5, 210 mg, 0.449 mmol) in EtOH (10.0 mL) was added K₂CO₃ (186 mg, 1.35 mmol) and the reaction mixture was stirred at 80° C. for 18 hours. The volatiles were removed under reduced pressure and the residue was treated with water (30 mL) and extracted with EtOAc (2×20). The combined organic layers were dried over anhydrous Na₂SO₄, filtered and concentrated under vacuum to afford the title compound (140 mg, yield: 92%) as a pale-yellow solid. LC-MS (Method B5′) m/z: [M+H]P: 315; rt: 1.81 min; purity: 93%. ¹H NMR (400 MHz, DMSO-d₆) δ 10.68 (s, 1H), 7.88 (dd, J=7.8, 1.3 Hz, 1H), 7.86-7.78 (m, 1H), 7.51 (t, J=7.8 Hz, 1H), 6.96 (s, 1H), 4.48-4.28 (m, 3H), 3.34 (d, J=12.8 Hz, 1H), 2.44 (s, 3H), 1.36 (t, J=7.1 Hz, 3H).

### Step 7: Synthesis of 1-fluoro-8-(hydroxymethyl)-3-methyl-5,7-dihydropyrido[3,4-a][3]benzazepin-6-one N45

At 0° C., to a stirred solution of ethyl 1-fluoro-3-methyl-6-oxo-5,7-dihydropyrido[4,3-d][3]benzazepine-8-carboxylate (Intermediate N45_6, 50.0 mg, 0.134 mmol) in dry THF (5.0 mL) was added dropwise a 4 M solution of LiBH₄ in THF (33 μL, 0.134 mmol) and the reaction mixture was allowed to reach room temperature overnight. At 0° C., additional LiBH₄ (4 M solution in THF, 33 μL, 0.134 mmol) was added and the reaction mixture was stirred at 0° C. for 5 h, before being diluted with water (30 mL) and extracted with EtOAc (2×15 mL). The combined organic layers were dried over anhydrous Na₂SO₄, filtered and concentrated under vacuum to afford the title compound (58 mg, yield: 88%) as pale yellow solid. LC-MS (Method A7) m/z: [M+Na]⁺: 295; rt: 1.30 min; purity: 86%. ¹H NMR (400 MHz, DMSO-d₆) δ 10.60 (s, 1H), 7.54-7.48 (m, 2H), 7.36 (t, J=7.7 Hz, 1H), 6.94 (s, 1H), 5.30 (dd, J=5.9, 4.7 Hz, 1H), 4.77 (dd, J=13.3, 5.9 Hz, 1H), 4.59 (dd, J=13.3, 4.6 Hz, 1H), 3.79 (d, J=12.9 Hz, 1H), 3.20 (d, J=12.9 Hz, 1H), 2.43 (s, 3H).

### Intermediate N46: (4-amino-3-bromo-6-methyl-2-pyridyl)methyl acetate

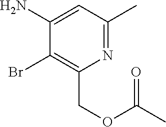

### Step 1 Synthesis of tert-butyl N-(5-bromo-2-methyl-4-pyridyl)-N-tert-butoxycarbonyl-carbamate N46_1

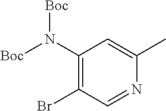 [SMILES: Cc1cc(N(C(=O)OC(C)(C)C)C(=O)OC(C)(C)C)c(Br)cn1]

To a solution of 4-amino-5-bromo-2-methylpyridine (717 mg, 3.75 mmol, 1 eq) in THF (37 mL) were added at room temperature DMAP (46 mg, 0.38 mmol, 0.1 eq) and di-tert-butyl dicarbonate (2.53 g, 11.3, 3 eq) and the reaction mixture was stirred at RT for 19 h. The reaction mixture was concentrated to dryness to afford a brown oil (1.77 g). The residue was purified by column chromatography on silica gel (using a gradient from 0% to 30% EtOAc in heptane as eluent) to afford the title compound as a white solid (1.36 g, yield: 89%). TLC (Ethyl acetate/Heptane 2/8) Rf: 0.26. LC-MS (Method A1) m/z [M+H]⁺ 389.1, rt: 4.52 min, purity: 99%

### Step 2: Synthesis of (4-amino-3-bromo-6-methyl-2-pyridyl)methyl acetate

Under inert atmosphere, a solution of tert-butyl N-(5-bromo-2-methyl-4-pyridyl)-N-tert-butoxycarbonyl-carbamate (Intermediate N46_1, 100 mg, 0.258 mmol), [bis(trifluoroacetoxy)iodo]benzene (133 mg, 0.309 mmol), (4,4′-di-t-butyl-2,2′-bipyridine)bis[3,5-difluoro-2-[5-trifluoromethyl-2-pyridinyl-kN)phenyl-kC]iridium(III) hexafluorophosphate (3 mg, 0.003 mmol) and acetoxyacetic acid (74 mg, 0.627 mmol) in dry dichloromethane (1.3 mL) was placed in a Pennoc reactor (450 nm; Fan speed=4000; Stirring rate=400; LED power=100%) for 3 h. The reaction mixture was concentrated under vacuum and diluted again in dry dichloromethane (0.5 mL). Trifluoroacetic acid (0.5 mL) was added dropwise and the reaction mixture was stirred at room temperature for 40 min. The reaction was concentrated under vacuum and purified by reverse phase chromatography (basic elution) to afford the title compound N46 (26 mg, yield: 39%) as a white solid. LC-MS (Method B1′) m/z [M+H]⁺: 260.9; rt: 1.68 min; purity: 90%.

### Intermediate N47: 4-bromo-6-methoxy-5-methyl-pyridin-3-amine

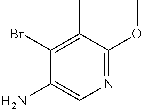

### Step 1: Synthesis of 4-bromo-2-methoxy-3-methyl-5-nitro-pyridine N47_1

At 0° C., to a solution of 4-bromo-2-methoxy-3-methylpyridine (200 mg, 0.95 mmol) in concentrated sulfuric acid (2.4 mL, 43 mmol), was added dropwise nitric acid (633 μL, 9.50 mmol). The reaction mixture was stirred for 10 min at room temperature and then heated at 100° C. for 2 h. After cooling to room temperature, the reaction mixture was poured into ice and water and the resulting precipitate was collected by filtration and rinsed with cold water. The filter cake was dissolved in dichloromethane, dried with MgSO₄, filtered off and concentrated under vacuum to give the title compound (140 mg, yield: 60%) as an off-white solid. LC-MS (method A1) m/z: [M+H]⁺: 249.2, rt: 1.46 min, purity>99%.

### Step 2: Synthesis of 4-bromo-6-methoxy-5-methyl-pyridin-3-amine N47

To a solution of 4-bromo-2-methoxy-3-methyl-5-nitropyridine (130 mg, 0.526 mmol) in absolute ethanol (1 mL) were added concentrated hydrochloric acid (368 μL, 4.40 mmol) and iron dust (104 mg, 1.84 mmol) and the reaction mixture was heated at 80° C. for 30 min. After cooling to room temperature, the reaction mixture was taken in a saturated NaHCO₃ solution and extracted twice with EtOAc. The combined organic layers were dried over MgSO₄, filtered off and concentrated under vacuum to afford the crude title product (120 mg, quantitative yield) as an orange oil which was taken to the next step without purification. LC-MS (method A1) m/z: [M+H]⁺: 217.1, rt: 1.06 min.

### Intermediate N48: methyl 2-(2-bromo-6-cyano-phenyl)acetate

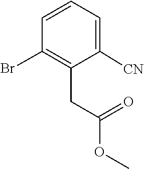

### Step 1: Synthesis of 2-(2-bromo-6-cyano-phenyl)acetic acid N48_1

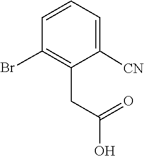 [SMILES: N#Cc1cccc(Br)c1CC(=O)O]

To a solution of LDA (19.5 mL, 39.3 mmol) in dry THF (100 mL) at −78° C., HMPA (6.2 mL, 35.7 mmol) and 3-cyano-2-methylbromobenzene (7 g, 35.7 mmol) were added and the reaction mixture was stirred at the same temperature for 2 h. C02 gas balloon was purged into the reaction mixture for 20 min. The reaction mixture was acidified with a 1N aqueous HCl solution to pH=2. The solvent was evaporated under reduced pressure and the residue was taken in ethyl acetate. The organic layer was separated, dried over sodium sulfate and concentrated under reduced pressure. The residue was purified by column chromatography on silica gel (using 5% ethyl acetate in hexanes as eluent) to afford the title compound. (4.2 g, yield: 47%). ¹H NMR (400 MHz, DMSO-d₆) δ 12.93 (bs, 1H) 8.00 (d, J=7.9 Hz, 1H), 7.90 (d, J=7.9 Hz, 1H), 7.43 (t, J=7.9 Hz, 1H), 3.96 (s, 2H).

### Step 2: Synthesis of methyl 2-(2-bromo-6-cyano-phenyl)acetate N48

To a solution of 2-(2-bromo-6-cyano-phenyl)acetic acid (1.0 g, 4.16 mmol) in dry methanol (21 mL), was added dropwise thionyl chloride (610 μL, 8.38 mmol) and the reaction mixture was heated at 40° C. for 4 h. The reaction mixture was concentrated to dryness and the residue was taken in saturated NaHCO₃ and extracted twice with EtOAc. The combined organic extracts were dried over MgSO₄, filtered off and concentrated under vacuum to give the title compound (1.05 g, yield: 99%) as a brown solid. ¹H NMR (400 MHz, DMSO-d₆) δ 8.02 (d, J=8.2 Hz, 1H), 7.93 (d, J=7.8 Hz, 1H), 7.46 (t, J=7.9 Hz, 1H), 4.07 (s, 2H), 3.68 (s, 3H). LC-MS (Method B4) m/z [M−H]⁻: 467.0; rt: 4.27 min; purity: 96%.

### Intermediate N49: methyl 2-(3-bromo-2-pyridyl)-2-fluoro-propanoate

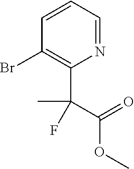

At −78° C., to a 2 M solution of LDA in THF (0.36 mL, 0.72 mmol) was added dropwise a solution of methyl 2-(3-bromopyridin-2-yl)propanoate (170 mg, 0.66 mmol) in dry THF (3.3 mL) and the reaction mixture was stirred at −78° C. for additional 10 min, then at 0° C. for 30 min. Again at −78° C., a solution of n-fluorobenzenesulfonimide (280 mg, 0.86 mmol) in dry THF (3.3 mL) was added dropwise and the reaction mixture was allowed to reach room temperature over 1 h. The reaction mixture was neutralized by addition of saturated NH₄Cl and extracted with EtOAc (3×). The combined organic layers were washed with brine, dried over MgSO₄, filtered off and concentrated to dryness. Purification by flash chromatography on silica gel (using a gradient of DCM/MeOH, from 100:0 to 90:10 as eluent) afforded the title product (123 mg, yield: 69%) as a colorless oil. LC-MS (method A1) m/z: [M+H]⁺: 263.9, rt: 1.20 min, purity: 98%. ¹H NMR (400 MHz, DMSO-d₆) δ 8.62 (d, J=4.6 Hz, 1H), 8.20 (d, J=8.0 Hz, 1H), 7.46 (dd, J=8.0, 4.6 Hz, 1H), 3.74 (s, 3H), 1.96 (d, J=22.6 Hz, 3H). LC-MS (Method B2) m/z [M−H]⁻: 477.0; rt: 4.60 min; purity: 95%.

### Intermediate N50: ethyl 2-(3-bromo-2-pyridyl)-3,3,3-trideuterio-propanoate

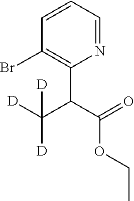 [SMILES: [2H]C([2H])([2H])C(C(=O)OCC)c1ncccc1Br]

At −78° C., to a 2 M solution of LDA in THF (1 mL, 2.10 mmol), further diluted in dry THF (8 mL), was added a solution of ethyl 2-(3-bromo-2-pyridyl)acetate (Intermediate N3_1, 500 mg, 2.0 mmol) in dry THF (2 mL). The resulting mixture was stirred at −78° C. for 10 min before addition of iodomethane-d3 (190 μL, 3.02 mmol). The reaction mixture was allowed to reach room temperature over 30 min, before being neutralized by addition of saturated NH₄Cl (5 mL) and extracted with EtOAc (3×20 mL). The combined organic layers were washed with brine (20 mL), dried over MgSO₄, filtered off and concentrated to dryness. Purification by flash chromatography on silica gel (using a gradient of heptane/EtOAc, from 100:0 to 50:50 as eluent) afforded the title compound (533 mg, quantitative yield) as a yellow oil. LC-MS (method A1) m/z: [M+H]⁺: 263.2, rt: 1.30 min, purity: 97%. ¹H NMR (400 MHz, DMSO-d₆) δ 8.52 (dd, J=4.6, 1.5 Hz, 1H), 8.08 (dd, J=8.0, 1.5 Hz, 1H), 7.27 (dd, J=8.0, 4.6 Hz, 1H), 4.28 (s, 1H), 4.07 (q, J=7.1 Hz, 2H), 1.11 (t, J=7.1 Hz, 3H). LC-MS (Method B2) m/z [M+H]⁺: 462.4; rt: 4.49 min; purity: 97%.

### Intermediate N51: methyl 2-(3-bromo-6-methyl-2-pyridyl)propanoate

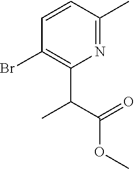

### Step 1: Synthesis of 2-(3-bromo-6-methylpyridin-2-yl)acetonitrile N51_1

At 0° C., to a solution of 3-bromo-2-fluoro-6-methylpyridine (5.00 g, 26.3 mmol) and acetonitrile (5.50 mL, 106 mmol) in dry toluene (100 mL) was added a 1 M solution of KHMDS in THF (32 mL, 31.6 mmol) and the reaction mixture was stirred at the same temperature for 1 h and then at room temperature for 16 h. The reaction mixture was diluted with EtOAc (100 mL) and washed with saturated NH₄Cl (2×40 mL) and brine (2×40 mL). The organic layer was separated, dried over anhydrous Na₂SO₄ and concentrated under vacuum. Purification by column chromatography on silica gel (using 5% EtOAc in hexanes as eluent) afforded the title compound (3.50 g, yield: 63%) as an off-white solid. LC-MS (method A5) m/z: [M+H]⁺: 212.7, rt: 1.72 min, purity: 84%. ¹H NMR (400 MHz, DMSO-d₆) δ 2.46 (s, 3H), 4.27 (s, 2H), 7.22 (d, J=8.0 Hz, 1H), 7.99 (d, J=8.0 Hz, 1H).

### Step 2: Synthesis of methyl 2-(3-bromo-6-methylpyridin-2-yl)acetate N51-2

A stirred solution of 2-(3-bromo-6-methylpyridin-2-yl)acetonitrile (2.25 g, 10.7 mmol) in a 3 M solution of HCl in MeOH (40 mL) was heated at 50° C. for 16 h. The solvent was removed under vacuum. The reaction mixture was diluted with DCM (50 mL), basified with saturated NaHCO₃ solution (40 mL) up to pH=9. The aqueous layer was separated and extracted with DCM (2×50 mL). The organic layer was separated, washed with brine (40 mL), dried over anhydrous Na₂SO₄, filtered off and concentrated under vacuum. Purification by column chromatography on silica gel (using a gradient of 0 to 5% EtOAc in hexanes as eluent) afforded the title compound (0.95 g, yield: 37%) as a pale-yellow oil. LC-MS (method A5) m/z: [M+H]⁺: 244.0, rt: 2.44 min, purity: 97%. ¹H NMR (400 MHz, DMSO-d₆) δ 2.41 (s, 3H), 3.63 (s, 3H), 3.94 (s, 2H), 7.14 (d, J=8.4 Hz, 1H), 7.93 (d, J=8.4 Hz, 1H).

### Step 3: Synthesis of methyl 2-(3-bromo-6-methyl-2-pyridyl)propanoate N_51

At −78° C., to a solution of methyl 2-(3-bromo-6-methyl-2-pyridyl)acetate (300 mg, 1.23 mmol) in dry THE (6 mL), was added a 2 M solution of LDA in THF (0.65 mL, 1.3 mmol) and the resulting mixture was stirred at −78° C. for 20 min before addition of iodomethane (116 μL, 1.84 mmol). The reaction mixture was allowed to reach room temperature for 30 min, before being neutralized by addition of saturated NH₄Cl (10 mL) and extracted with EtOAc (3×30 mL). The combined organic layers were washed with brine (20 mL), dried over MgSO₄, filtered off and concentrated to dryness to afford the title product (326 mg, yield: 94%) as a light brown oil. LC-MS (method A1) m/z: [M+H]⁺: 260.1, rt: 1.32 min, purity: 92%. ¹H NMR (400 MHz, DMSO-d₆) δ 7.92 (d, J=8.2 Hz, 1H), 7.12 (d, J=8.2 Hz, 1H), 4.27 (q, J=7.2 Hz, 1H), 3.58 (s, 3H), 2.40 (s, 3H), 1.39 (d, J=7.2 Hz, 3H). LC-MS (Method B2) m/z [M+H]⁺: 473.1; rt: 4.90 min; purity: 97%.

### Intermediate N52: 4-amino-3-bromo-6-methyl-pyridine-2-carbonitrile

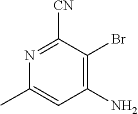

### Step 1: Synthesis of tert-butyl N-(5-bromo-2-methyl-4-pyridyl)-N-tert-butoxycarbonyl-carbamate N52_1

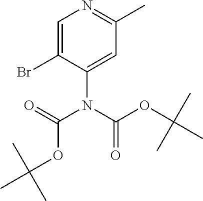 [SMILES: Cc1cc(N(C(=O)OC(C)(C)C)C(=O)OC(C)(C)C)c(Br)cn1]

To a solution of 4-amino-5-bromo-2-methylpyridine (2.0 g, 10.0 mmol) in dry THF (105 mL) were added 4-dimethylaminopyridine (129 mg, 1.05 mmol) and di-tert-butyl dicarbonate (7 g, 31.1 mmol) and the reaction mixture was stirred at room temperature for 5 h. The reaction mixture was concentrated under vacuum and purified by column chromatography on silica gel (using a gradient of 0% to 30% EtOAc in heptane as eluent) to afford he title product (2.3 g, yield: 54%) as a white solid. LC-MS (Method B4) m/z [M+H]⁺: 389.0; rt: 4.49 min; purity: 98%.

### Step 2: Synthesis of tert-butyl N-(5-bromo-2-methyl-1-oxido-pyridin-1-ium-4-yl)-N-tert-butoxycarbonyl-carbamate N52_2

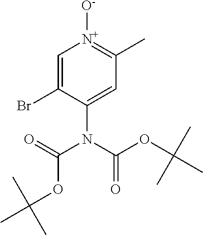 [SMILES: Cc1cc(N(C(=O)OC(C)(C)C)C(=O)OC(C)(C)C)c(Br)c[n+]1[O-]]

To a solution of tert-butyl N-(5-bromo-2-methyl-4-pyridyl)-N-tert-butoxycarbonyl-carbamate (Intermediate N52_1, 3.78 g, 8.78 mmol) in dry dichloromethane (90 mL) was added, at 0° C., 3-chloroperoxybenzoic acid (3.3 g, 13.0 mmol) and the reaction mixture was stirred at room temperature for 20 h. To the reaction mixture were added water and dichloromethane, the layers were separated, and the organic layer was concentrated to dryness. The crude solid was purified by flash chromatography on silica gel (using a gradient of heptane/EtOAc from 80/20 to 20/80 as eluent) to afford the title product (3.1 g, yield: 82%) as a white solid. LC-MS (Method A1) m/z [M+H]⁺: 403.3; rt: 1.27 min; purity: 94%.

### Step 3: Synthesis of tert-butyl N-(3-bromo-2-cyano-6-methyl-4-pyridyl)-N-tert-butoxycarbonyl-carbamate N52_3

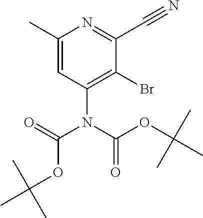 [SMILES: Cc1cc(N(C(=O)OC(C)(C)C)C(=O)OC(C)(C)C)c(Br)c(C#N)n1]

At room temperature, to a solution of tert-butyl N-(5-bromo-2-methyl-1-oxido-pyridin-1-ium-4-yl)-N-tert-butoxycarbonyl-carbamate (Intermediate N52_2, 3.0 g, 6.9 mmol) in dry acetonitrile (70 mL) were added trimethylsilyl cyanide (3.6 mL, 27.0 mmol) and triethylamine (2.4 mL, 17.0 mmol) and the reaction mixture was stirred at 90° C. for 2.5 h. To the reaction mixture were added again triethylamine (2.4 mL, 17.0 mmol) and trimethylsilyl cyanide (3.6 mL, 27.0 mmol) and the reaction mixture was heated again at 90° C. for 18 h. After cooling to room temperature, the reaction mixture was partitioned between a saturated aqueous NaHCO₃ solution and EtOAc and the two layers were separated. The aqueous layer was extracted twice with EtOAc and the combined organic layers were dried over Na₂SO₄, filtered off and concentrated to dryness. Purification by flash chromatography on silica gel (using a gradient of heptane/EtOAc from 100/0 to 50/50 as eluent) afforded the title product (2.15 g, yield: 66%) as a white solid. LC-MS (Method A1) m/z [M+H]⁺: 414.3; rt: 1.61 min; purity: 87%.

### Step 4: Synthesis of 4-amino-3-bromo-6-methyl-pyridine-2-carbonitrile N52

To a solution of tert-butyl N-(3-bromo-2-cyano-6-methyl-4-pyridyl)-N-tert-butoxycarbonyl-carbamate (Intermediate N52_3, 2.2 g, 5.3 mmol) in dichloromethane (45 mL) was added a 4 M solution of hydrochloric acid in 1,4-dioxane (22 mL) and the reaction mixture was stirred at room temperature for 24 h. The reaction mixture was concentrated to dryness to give a crude yellow solid which was taken in water and a saturated Na₂CO₃ aqueous solution. The aqueous layer was extracted with EtOAc (3×) and the combined organic layer were dried over Na₂SO₄, filtered and concentrated to dryness. Purification by flash chromatography on silica gel (using a gradient of DCM/EtOAc from 100/0 to 50/50 as eluent) afforded the title compound (1.1 g, yield: 97%) as an off-white solid. LC-MS (Method A1) m/z [M+H]⁺: 212.1; rt: 0.87 min; purity>99%. ¹H NMR (400 MHz, DMSO-d₆) δ 6.76 (bs, 2H), 6.69 (s, 1H), 2.27 (s, 3H).

### Intermediate N53: 3-methyl-9-oxo-4,8,12-triazatricyclo[9.4.0.0^{2,7}]pentadeca-1(11),2(7),3,5,12,14-hexaene-5-carbonitrile

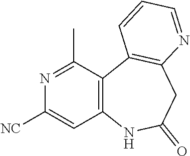 [SMILES: Cc1nc(C#N)cc2c1-c1cccnc1CC(=O)N2]

### Step 1: Synthesis of 4-amino-6-methyl-pyridine-2-carbonitrile N53_I

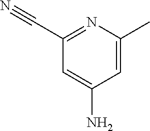

To a stirred solution of 6-methyl-4-nitro-pyridine-2-carbonitrile (1.00 g, 6.13 mmol) in AcOH (16 mL) was added iron dust (856 mg, 15.3 mmol) and the reaction mixture was stirred for 5 h at room temperature. Water (15 mL) was added, and the mixture was basified to pH 8 with an aq. solution of NaOH (6M). The aqueous phase was then extracted with EtOAc (3×100 mL). The combined organic extracts were dried over sodium sulfate, filtered and concentrated under vacuum. The residue was dry loaded onto silica and purified by flash chromatography on silica gel (using a gradient of 0 to 10% MeOH in DCM as eluent) to afford the title compound (549 mg, yield: 67%) as a light beige solid. LC-MS (Method A7) m/z [M+H]⁺: 134.2; rt: 0.16 min, purity: >99%. ¹H NMR (400 MHz, DMSO-d₆) δ 6.80 (d, J=2.1 Hz, 1H), 6.55-6.52 (m, 1H), 6.47 (s, 2H), 2.26 (s, 3H).

### Step 2: Synthesis of 4-amino-5-bromo-6-methyl-pyridine-2-carbonitrile N53_2

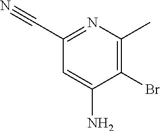

A solution of 4-amino-6-methyl-pyridine-2-carbonitrile (Intermediate N53_1, 549 mg, 4.13 mmol) in AcOH (5 mL) was treated dropwise with a solution of bromine (0.21 mL, 4.13 mmol) in AcOH (1 mL) at RT. After 1 h, the resulting slurry was treated with 40 mL of 20% NaOH solution and extracted with EtOAc (3×100 mL). The combined organic extracts were dried over magnesium sulfate, filtered, and concentrated under vacuum. The residue was purified by flash chromatography on silica gel (using a gradient of 0 to 5% MeOH in DCM as eluent). The resulting solid was then slurred in a 1:1 solution of EtOAc/iso-hexane and the insoluble material was filtered off. The filtrate was concentrated under vacuum to afford the title compound (155 mg, yield: 17%) as a pale beige solid. LC-MS (Method A7) m/z [M+H]⁺: 212.0/214.0; rt: 1.35 min, purity: >99%. ¹H NMR (400 MHz, DMSO-d₆) δ 7.00 (s, 1H), 6.79 (s, 2H), 2.46 (s, 3H).

### Step 3: Synthesis of 3-methyl-9-oxo-4,8,12-triazatricyclo[9.4.0.0^{2,7}]pentadeca-1(11),2(7),3,5,12,14-hexaene-5-carbonitrile N53

A mixture of 4-amino-5-bromo-6-methyl-pyridine-2-carbonitrile (Intermediate N53_2, 155 mg, 0.729 mmol), ethyl 2-[3-(4,4,5,5-tetramethyl-1,3,2-dioxaborolan-2-yl)-2-pyridyl]acetate (Intermediate N25_2, 304 mg, 1.02 mmol) and CsF (310 mg, 2.04 mmol) in dry 1,4-dioxane (14 mL) was degassed by bubbling through nitrogen for 10 min. Bis(di-tert-butyl(4-dimethylaminophenyl)phosphine)dichloropalladium(II) (26 mg, 0.037 mmol) was added and nitrogen was bubbled through for a further 5 min, then the reaction mixture was stirred at reflux overnight. The reaction mixture was allowed to cool to room temperature, then filtered through a bed of Celite® and washed with EtOAc (100 mL). The filtrate was concentrated under vacuum and the residue dry loaded onto silica and purified by flash chromatography on silica gel (using a gradient of 0 to 100% EtOAc in iso-hexane as eluent) to afford the title compound (28 mg, yield: 9%) as a yellow gum. LC-MS (Method A7) m/z [M+H]⁺: 251.1; rt: 1.13 min, purity: 60%.

### Intermediate N54: 3,5-dichloro-4,8,12-triazatricyclo[9.4.0.0^{2,7}]pentadeca-1(11),2(7),3,5,12,14-hexaen-9-one

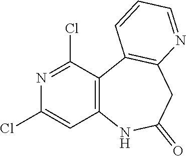 [SMILES: O=C1Cc2ncccc2-c2c(cc(Cl)nc2Cl)N1]

### Step 1: Synthesis of 2,6-dichloro-3-iodo-pyridin-4-amine N54_1

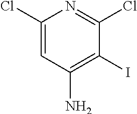

2,6-Dichloropyridin-4-amine (1.00 g, 5.83 mmol) was dissolved in acetonitrile (40 mL) and N-iodosuccinimide (1.14 g, 6.41 mmol) was added at 0° C. The ice bath was removed after 10 min and the reaction mixture was stirred at 80° C. for 24 h. The reaction mixture was concentrated under vacuum and the residue was purified by flash chromatography on silica gel (using a gradient of 0 to 50% EtOAc in iso-hexane as eluent) to afford the title compound (1.25 g, yield: 72%) as a beige solid. ¹H NMR (400 MHz, CDCl₃) δ 6.53 (s, 1H), 5.00 (s, 2H). LC-MS (Method A7) m/z [M+H]⁺: 288.9/290.9; rt: 1.85 min, purity: 99%.

### Step 2: Synthesis of ethyl 2-[3-(4-amino-2,6-dichloro-3-pyridyl)-2-pyridyl]acetate N54_2

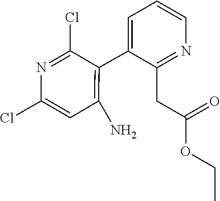 [SMILES: CCOC(=O)Cc1ncccc1-c1c(N)cc(Cl)nc1Cl]

A mixture of 2,6-dichloro-3-iodo-pyridin-4-amine (Intermediate N54_1, 233 mg, 0.807 mmol), ethyl 2-[3-(4,4,5,5-tetramethyl-1,3,2-dioxaborolan-2-yl)-2-pyridyl]acetate (Intermediate N25_2, 580 mg, 1.21 mmol), CsF (343 mg, 2.26 mmol) and bis(di-tert-butyl(4-dimethylaminophenyl)phosphine)dichloropalladium(II) (29 mg, 0.040 mmol) in dry 1,4-dioxane (28 mL) and water (1.4 mL) was purged with nitrogen for 15 min and then stirred at reflux overnight. The reaction was allowed to cool to RT, filtered through a bed of Celite® and washed with EtOAc (50 mL). The filtrate was concentrated under vacuum to afford the title compound as a brown oil. The product was taken crude to the next step without purification. LC-MS (Method B5′) m/z [M+H]⁺: 326.1/328.1; rt: 1.60 min, purity: 42%.

### Step 3: Synthesis of 3,5-dichloro-4,8,12-triazatricyclo[9.4.0.0^{2,7}]pentadeca-1(11),2(7),3,5,12,14-hexaen-9-one N54

Ethyl 2-[3-(4-amino-2,6-dichloro-3-pyridyl)-2-pyridyl]acetate (Intermediate N54_2, 263 mg, 0.806 mmol) was dissolved in EtOH (3.9 mL) and K₂CO₃ (223 mg, 1.61 mmol) was added. The reaction mixture was heated at 80° C. for 1 h then was allowed to cool to room temperature and water (50 mL) was added. The aqueous phase was extracted with EtOAc (3×50 mL) and the combined organic extracts were dried over sodium sulfate, filtered and concentrated under vacuum. The residue was purified by flash chromatography on silica gel (using a gradient of 0 to 10% MeOH in DCM as eluent) to afford the title compound (64 mg, yield: 28%) as an off-white solid. LC-MS (Method A7) m/z [M+H]⁺: 280.0; rt: 1.48 min, purity>99%. ¹H NMR (400 MHz, DMSO-d₆) δ 10.84 (s, 1H), 8.63 (dd, J=4.8, 1.7 Hz, 1H), 8.25 (dd, J=8.0, 1.7 Hz, 1H), 7.49 (dd, J=8.0, 4.8 Hz, 1H), 7.30 (s, 1H), 3.94 (d, J=12.7 Hz, 1H), 3.61 (d, J=12.7 Hz, 1H).

### Intermediate N55

### 3,12-difluoro-5,10-dimethyl-4,8,14-triazatricyclo[9.4.0.0^{2,7}]pentadeca-1(11),2(7),3,5,12,14-hexaen-9-one

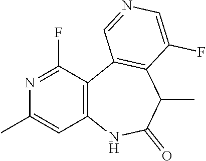 [SMILES: Cc1cc2c(c(F)n1)-c1cncc(F)c1C(C)C(=O)N2]

### Step 1: Synthesis of ethyl 2-(3-bromo-5-fluoro-4-pyridyl)propanoate N55_1

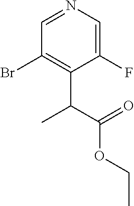

Ethyl 2-(3-bromo-5-fluoro-4-pyridyl)acetate (Intermediate N65_1, 1.00 g, 3.62 mmol) was dissolved in THF (10 mL) and cooled to 0° C. A 1M solution of lithium bis(trimethylsilyl)amide in THF (4.17 mL, 4.17 mmol) was added dropwise and the reaction mixture was stirred for 30 min at 0° C. Iodomethane (669 mg, 4.71 mmol) was added and the reaction mixture was stirred for a further 1 h at 0° C. The solution was concentrated under vacuum and the residue was purified by column chromatography on silica gel (using a gradient of 0 to 60% MTBE in iso-hexane as eluent) to afford the title compound (1.0 g, yield: 98%) as a colourless oil. LC-MS (Method A7) m/z [M+H]⁺: 276.1/278.1; rt: 1.99 min; purity: 98%. ¹H NMR (400 MHz, CDCl₃) δ 8.55 (s, 1H), 8.38-8.33 (m, 1H), 4.28-4.08 (m, 3H), 1.51 (dd, J=7.3, 0.7 Hz, 3H), 1.21 (t, J=7.1 Hz, 3H).

### Step 2: Synthesis of ethyl 2-[3-(4-amino-2-fluoro-6-methyl-3-pyridyl)-5-fluoro-4-pyridyl]propanoate N55_2

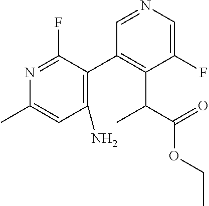 [SMILES: CCOC(=O)C(C)c1c(F)cncc1-c1c(N)cc(C)nc1F]

A mixture of ethyl 2-(3-bromo-5-fluoro-4-pyridyl)propanoate (Intermediate N55_1, 75 mg, 0.272 mmol), 2-fluoro-6-methyl-3-(4,4,5,5-tetramethyl-1,3,2-dioxaborolan-2-yl)pyridin-4-amine (Intermediate N16_1, 77 mg, 0.299 mmol) and CsF (124 mg, 0.815 mmol) in 1,4-dioxane (7.5 mL) and water (1.0 mL) was degassed with nitrogen for 10 min before the addition of bis(di-tert-butyl(4-dimethylaminophenyl)phosphine)dichloropalladium(II) (19 mg, 0.027 mmol). The reaction mixture was heated at 90° C. for 3 hours. The reaction mixture was evaporated to dryness and the residue was purified by flash chromatography on silica gel (using a gradient of 0 to 100% EtOAc in iso-hexane as eluent) to afford the title compound (72 mg, yield: 58%). LC-MS (Method A7) m/z [M+H]⁺: 322.2; rt: 1.60 and 1.70 min; purity: 98%.

### Step 3: Synthesis of 3,12-difluoro-5,10-dimethyl-4,8,14-triazatricyclo[9.4.0.0²⁷]pentadeca-1(11),2,4,6,12,14-hexaen-9-one N55

A suspension of ethyl 2-[3-(4-amino-2-fluoro-6-methyl-3-pyridyl)-5-fluoro-4-pyridyl]propanoate (Intermediate N55_2, 70 mg, 0.213 mmol) and K₂CO₃ (88 mg, 0.640 mmol) in EtOH (10 mL) was stirred at 80° C. for 2 h. The reaction mixture was allowed to cool to room temperature and the solvent was removed under vacuum. To the residue was added water (10 mL) and the pH was adjusted to 4-5 by the addition of acetic acid. The resulting precipitate was collected by filtration, washed with water (~2 mL) and dried under vacuum to give the title compound (57 mg, yield: 95%) as a white solid. LC-MS (Method A7) m/z [M+H]⁺: 276.2; rt: 1.47 min; purity: 98%.

### Intermediate N56: 3-fluoro-5,10-dimethyl-4,8,12,13-tetrazatricyclo[9.4.0.0^{2,7}]pentadeca-1(11),2(7),3,5,12,14-hexaen-9-one

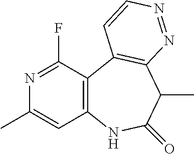

### Step 1: Synthesis of ethyl 2-(4-benzyloxypyridazin-3-yl)propanoate N56_1

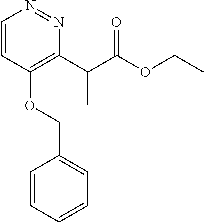 [SMILES: CCOC(=O)C(C)c1nnccc1OCc1ccccc1]

Ethyl 2-(4-(benzyloxy)pyridazin-3-yl)acetate (Intermediate N34_3, 1.0 g, 3.67 mmol) was dissolved in dry THF (20 mL) and cooled to 0° C. A 1M solution of lithium bis(trimethylsilyl)amide in THF (4.0 mL, 4.0 mmol) was added dropwise and the reaction mixture was stirred for 30 min at 0° C. Iodomethane (680 mg, 4.78 mmol) was added and the reaction mixture was stirred for a further 1 h at 0° C. The reaction mixture was concentrated under vacuum and purified by flash chromatography on silica gel (using neat EtOAc as eluent) to give the title compound (1.0 g, yield: 79%) as a pale yellow oil. LC-MS (Method B5′) m/z [M+H]⁺: 287.2; rt: 1.65 min; purity: 90%. ¹H NMR (400 MHz, DMSO-d₆) δ 8.97 (d, J=5.9 Hz, 1H), 7.43-7.37 (m, 5H), 7.34 (d, J=5.9 Hz, 1H), 5.26 (d, J=3.4 Hz, 2H), 4.23 (q, J=7.2 Hz, 1H), 3.99 (qd, J=7.1, 2.4 Hz, 2H), 1.48 (d, J=7.1 Hz, 3H), 1.03 (t, J=7.1 Hz, 3H).

### Step 2: Synthesis of ethyl 2-(4-hydroxypyridazin-3-yl)propanoate N56_2

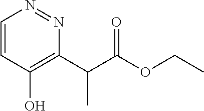

To a solution of ethyl 2-(4-benzyloxypyridazin-3-yl)propanoate (Intermediate N56_1, 1.0 g, 3.42 mmol) in EtOH (60 mL) was added 10% Pd/C (182 mg) and the reaction mixture was stirred under hydrogen atmosphere (P=5 Bars) for 12 h. The catalyst was removed by filtration and the filtrate was evaporated to dryness and purified by flash chromatography on silica gel (using a gradient of 0 to 5% MeOH in DCM as eluent) to give the title compound (472 mg, yield: 59%) as a colorless solid. LC-MS (Method B5′) m/z [M+H]P: 197.2; rt: 0.89 min; purity: 90%. ¹H NMR (400 MHz, DMSO-d₆) δ 13.07 (s, 1H), 8.18 (d, J=7.4 Hz, 1H), 6.25 (d, J=7.4 Hz, 1H), 4.06-4.00 (m, 2H), 3.88 (q, J=7.2 Hz, 1H), 1.31 (d, J=7.2 Hz, 3H), 1.11 (t, J=7.1 Hz, 3H).

### Step 3: Synthesis of ethyl 2-(4-chloropyridazin-3-yl)propanoate N56_3

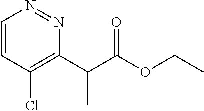

To a solution of ethyl 2-(4-hydroxypyridazin-3-yl)propanoate (Intermediate N56_2, 566 mg, 2.83 mmol) in acetonitrile (25 mL) was added phosphorus oxychloride (0.65 mL, 7.07 mmol) and the reaction was heated at 80° C. for 1 h. The reaction mixture was allowed to cool to room temperature, poured onto a solution of sat. aq. sodium bicarbonate (30 mL) and extracted with EtOAc (2×20 mL). The organic extracts were combined, dried over sodium sulfate, filtered and concentrated under vacuum. The residue was purified by flash chromatography on silica gel (using a gradient of 0 to 60% EtOAc in iso-hexane as eluent) to give the title compound (457 mg, yield: 72%) as an orange oil. LC-MS (Method A7) m/z [M+H]⁺: 215.1/217.1; rt: 1.44 min; purity: 98%. ¹H NMR (400 MHz, CDCl₃) δ 9.01 (d, J=5.5 Hz, 1H), 7.51 (d, J=5.5 Hz, 1H), 4.42 (q, J=7.2 Hz, 1H), 4.18 (q, J=7.1 Hz, 2H), 1.71 (d, J=7.2 Hz, 3H), 1.20 (t, J=7.1 Hz, 3H).

### Step 4: Synthesis of ethyl 2-[4-(4-amino-2-fluoro-6-methyl-3-pyridyl)pyridazin-3-yl]propanoate N56_4

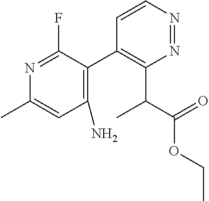 [SMILES: CCOC(=O)C(C)c1nnccc1-c1c(N)cc(C)nc1F]

A mixture of ethyl 2-(4-chloropyridazin-3-yl)propanoate (Intermediate N56_3, 400 mg, 1.66 mmol), 2-fluoro-6-methyl-3-(4,4,5,5-tetramethyl-1,3,2-dioxaborolan-2-yl)pyridin-4-amine (Intermediate N16_1, 559 mg, 2.00 mmol) and aq. K₂CO₃ (3.5 mL, 4.99 mmol) in 1,4-dioxane (50 mL) was purged with nitrogen for 10 min before SPhos Pd(crotyl)Cl (CAS: 1798781-99-3, 101 mg, 0.166 mmol) was added. The reaction mixture was heated at 90° C. for 1 h. The reaction mixture was allowed to cool to room temperature, poured onto water (150 mL) and extracted with EtOAc (2×100 mL). The organic extracts were combined, dried over sodium sulfate, filtered and concentrated under vacuum. The residue was purified by flash chromatography on silica gel (using a gradient of 0 to 100% EtOAc in iso-hexane as eluent) to give the title compound (402 mg, yield: 69%) as a pale yellow solid. LC-MS (Method B5′) m/z [M+H]⁺: 305.2; rt: 1.34 min; purity: 95%.

### Step 5: Synthesis of 3-fluoro-5,10-dimethyl-4,8,12,13-tetrazatricyclo[9.4.0.0^{2,7}]pentadeca-1(11),2(7),3,5,12,14-hexaen-9-one N56

A suspension of ethyl 2-[4-(4-amino-2-fluoro-6-methyl-3-pyridyl)pyridazin-3-yl]propanoate (Intermediate N56 4. 400 mg, 1.25 mmol) and K₂CO₃ (518 mg, 3.75 mmol) in EtOH (20.0 mL) was stirred at 80° C. for 2 h. The reaction mixture was evaporated to dryness and the residue taken up into water (20 mL). The pH was adjusted to 5 by the addition of AcOH (~3 mL) and the mixture was extracted with EtOAc (2×50 mL). The organic extracts were combined, dried over sodium sulfate, filtered and concentrated under vacuum to give the title compound (272 mg, yield: 76%) as a buff colored solid. LC-MS (Method A7) m/z [M+H]⁺: 259.1; rt: 1.14 min; purity: 90%. ¹H NMR (400 MHz, DMSO-d₆) δ 10.99 (s, 1H), 9.31 (d, J=5.3 Hz, 1H), 7.97 (dd, J=5.3, 4.2 Hz, 1H), 7.02 (s, 1H), 3.91 (q, J=6.6 Hz, 1H), 2.47 (s, 3H), 1.63 (d, J=6.6 Hz, 3H).

### Intermediate N57: 3-fluoro-5,10-dimethyl-4,8,14-triazatricyclo[9.4.0.0^{2,7}]pentadeca-1(11),2(7),3,5,12,14-hexaen-9-one

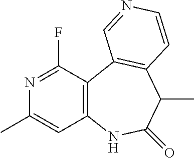

### Step 1: Synthesis of methyl 2-(3-bromo-4-pyridyl)acetate N57_1

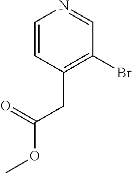

A solution of 3-bromo-4-methylpyridine (3.2 mL, 29.0 mmol) in anhydrous THF (60 mL) was added dropwise to a 1 M solution of lithium bis(trimethylsilyl)amide in THF (87 mL, 87.0 mmol). The reaction mixture was stirred for 1.5 h at RT and dimethyl carbonate (3.9 mL, 46.5 mmol) was added. The reaction mixture was stirred for 15 h at RT before being concentrated under vacuum. The residue was partitioned between EtOAc (100 mL) and water (50 mL) and the aqueous phase was extracted with EtOAc (2×40 mL). The combined organic extracts were dried over sodium sulfate, filtered and concentrated under vacuum. The residue was purified by flash chromatography on silica gel (using a gradient of 0 to 100% EtOAc in iso-hexane as eluent) to afford the title compound (3.92 g, yield: 58%) as a light yellow oil. LC-MS (Method A7) m/z [M+H]⁺: 230.0/232.0; rt: 1.22 min; purity: 99%. ¹H NMR (400 MHz, DMSO-d₆) δ 8.73 (s, 1H), 8.58-8.45 (m, 1H), 7.51-7.44 (m, 1H), 3.89 (s, 2H), 3.65 (s, 3H).

### Step 2: Synthesis of methyl 2-(3-bromo-4-pyridyl)propanoate N57_2

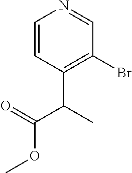

To a solution of methyl 2-(3-bromo-4-pyridyl)acetate (Intermediate N57_1, 3.92 g, 16.9 mmol) in dry THF (50 mL) cooled to −78° C. was added a 1M solution of LiHMDS in THF (20.2 mL, 20.2 mmol). The reaction mixture was stirred at −78° C. for 10 min and then iodomethane (1.4 mL, 21.9 mmol) was added dropwise. The reaction mixture was stirred at −78° C. for 1 h, then allowed to warm to 0° C. and stirred for 1 h. The reaction mixture was treated with a 1M aq. NH₄Cl solution (10 mL), followed by brine (10 mL) and was extracted with EtOAc (3×20 mL). The combined organic layers were dried over sodium sulfate, filtered and concentrated under vacuum. The residue was purified by flash chromatography on silica gel (using a gradient of 0 to 100% EtOAc in iso-hexane as eluent) to afford the title compound (3.60 g, yield: 92%) as a yellow/orange liquid. LC-MS (Method A7) m/z [M+H]⁺: 244.1/246.1; rt: 1.53 min; purity: 99%. ¹H NMR (400 MHz, DMSO-d₆) δ 8.73 (s, 1H), 8.55-8.49 (m, 1H), 7.43-7.38 (m, 1H), 4.12 (q, J=7.2 Hz, 1H), 3.63 (s, 3H), 1.44 (d, J=7.2 Hz, 3H).

### Step 3: Synthesis of 3-fluoro-5,10-dimethyl-4,8,14-triazatricyclo[9.4.0.0^{2,7}]pentadeca-1(11),2(7),3,5,12,14-hexaen-9-one N57

A stirring mixture of 2-fluoro-6-methyl-3-(4,4,5,5-tetramethyl-1,3,2-dioxaborolan-2-yl)pyridin-4-amine (Intermediate N16_1, 200 mg, 0.682 mmol), methyl 2-(3-bromo-4-pyridyl)propanoate (Intermediate N57_2, 290 mg, 1.09 mmol), bis(di-tert-butyl(4-dimethylaminophenyl)phosphine)dichloropalladium(II) (48 mg, 0.068 mmol) and CsF (311 mg, 2.05 mmol) in 1,4-dioxane (10 mL) and water (2 mL) was degassed with nitrogen for 5 min and then heated at 90° C. for 2 h. The reaction mixture was cooled down to room temperature, filtered through a small pad of celite, rinsed with EtOAc (40 mL) and the filtrate was concentrated under vacuum. The residue was dissolved in EtOH (10 mL) and potassium carbonate (189 mg, 1.36 mmol) was added. The reaction mixture was stirred at 80° C. overnight. The reaction mixture was cooled down to room temperature and filtered off. The filtrate was concentrated under vacuum and purified by flash chromatography on silica gel (using a gradient of 0 to 10% MeOH in DCM as eluent) to afford the title compound (183 mg, yield: 80%) as a yellow gum. LC-MS (Method A7) m/z [M+H]⁺: 258.2; rt: 1.01 min; purity: 77%.

### Intermediate N58: 3-fluoro-5-methoxy-10-methyl-4,8,12-triazatricyclo[9.4.0.0^{2,7}]pentadeca-1(11),2(7),3,5,12,14-hexaen-9-one

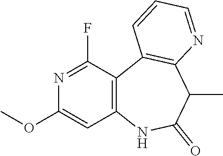

### Step 1: Synthesis of ethyl 2-[3-(4-amino-2-fluoro-6-methoxy-3-pyridyl)-2-pyridyl]propanoate N58_1

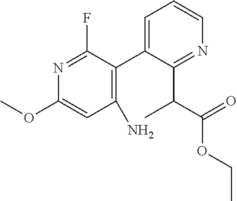 [SMILES: CCOC(=O)C(C)c1ncccc1-c1c(N)cc(OC)nc1F]

A suspension of ethyl 2-(3-bromo-2-pyridyl)propanoate (Intermediate N68_2, 2.90 g, 11.0 mmol), 2-fluoro-6-methoxy-3-(4,4,5,5-tetramethyl-1,3,2-dioxaborolan-2-yl)pyridin-4-amine (Intermediate N40_1, 4.56 g, 13.8 mmol) and CsF (5.85 g, 38.5 mmol) in dry toluene (12.0 mL), EtOH (6.0 mL) and water (6.0 mL) was purged with nitrogen for 10 min and PEPPSI™_IPr (CAS 905459-27-0, 748 mg, 1.10 mmol) was then added. The reaction mixture was purged with nitrogen for an additional 5 min and was heated at 80° C. for 16 h. The reaction mixture was cooled to room temperature, filtered through a pad of Celite® and washed with EtOAc (50 mL). The filtrate was concentrated under vacuum and the residue was purified by flash chromatography on silica gel (using a gradient of 0 to 5% MeOH in DCM as eluent) to afford the title compound as a yellow solid (1.73 g, yield: 42%). LC-MS (Method A7) m/z [M+H]⁺: 320.2; rt: 1.71 min; purity: 87%.

### Step 2: Synthesis of 3-fluoro-5-methoxy-10-methyl-4,8,12-triazatricyclo[9.4.0.0^{2,7}]pentadeca-1(11),2(7),3,5,12,14-hexaen-9-one N58

To a solution of ethyl 2-[3-(4-amino-2-fluoro-6-methoxy-3-pyridyl)-2-pyridyl]propanoate (Intermediate N58_1, 1.96 g, 5.52 mmol) in dry THF (50.0 mL) was added dropwise a 1M solution of lithium bis(trimethylsilyl)amide in THF (11.0 mL, 11.0 mmol) and the reaction mixture was stirred for 1 h at room temperature. The reaction mixture was treated with ice-water (10 mL) and extracted with EtOAc (3×20 mL) and the combined organic extracts were dried over magnesium sulfate, filtered and concentrated under vacuum to afford the title compound as a light brown powder (1.59 g, quantitative yield). LC-MS (Method A7) m/z [M+H]⁺: 274.1; rt: 1.59 min; purity: 96%. ¹H NMR (400 MHz, DMSO-d₆) δ 10.72 (s, 1H), 8.62 (dd, J=4.7, 1.7 Hz, 1H), 8.04 (ddd, J=7.9, 4.7, 1.7 Hz, 1H), 7.44 (dd, J=7.9, 4.7 Hz, 1H), 6.54 (s, 1H), 3.89 (s, 3H), 3.72-3.66 (m, 1H), 1.47 (d, J=6.6 Hz, 3H). The racemate was separated by chiral chromatography (SFC, Chiralpak IG from Daicel, CO₂+Methanol 20%). Chiral purity 100%. Rt=1.78 min (first eluting enantiomer N58_A). For information, second eluting enantiomer N58_B rt=2.24 min. Both measured by HPLC, Chiralpak IG from Daicel, Solvent: ACN100%—DEA 0.1%).

### Intermediate N59a and N59b: 3-fluoro-4-methoxy-10-methyl-5,8,12-triazatricyclo[9.4.0.0^{2,7}]pentadeca-1(11),2,4,6,12,14-hexaen-9-one N59a and 5-fluoro-4-methoxy-10-methyl-3,8,12-triazatricyclo[9.4.0.0^{2,7}]pentadeca-1(11),2,4,6,12,14-hexaen-9-one N59b

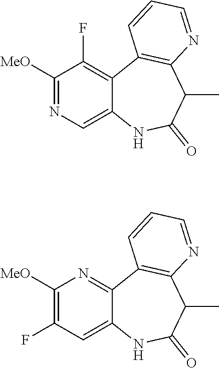 [SMILES: COc1ncc2c(c1F)-c1cccnc1C(C)C(=O)N2 ; COc1nc2c(cc1F)NC(=O)C(C)c1ncccc1-2]

To a solution of 3-amino-5-fluoro-6-methoxypyridine (500 mg, 3.52 mmol) and bispinacolatodiboron (1.79 g, 7.04 mmol) in dry THF (2.5 mL) at room temperature was added a suspension of 4,4′-di-tert-butyl-2,2′-dipyridyl (29 mg, 0.11 mmol) and (1,5-cyclooctadiene)(methoxy)iridium(I) dimer (CAS 12148-71-9, 35 mg, 0.05 mmol) in dry THF (1 mL) and the reaction mixture was stirred at 80° C. for 16 h. The reaction mixture was then cooled down to room temperature and slowly added to a solution of ethyl 2-(3-bromo-2-pyridyl)propanoate (Intermediate N68_2, 908 mg, 3.52 mmol) and K₂CO₃ (1.47 g, 10.6 mmol) in 1,4-dioxane (17.0 mL) and water (1.0 mL). The solution was degassed with nitrogen for 5 min before the addition of Pd[(Amphos)₂Cl]₂ (CAS 887919-35-9, 125 mg, 0.18 mmol). The reaction mixture was then stirred at 100° C. for 4 h. After cooling to room temperature, the reaction mixture was diluted with EtOAc (100 mL), filtered through a pad of Celite® and the filtrate was concentrated to dryness. The residue was then dissolved in dry toluene (18 mL) and a 1.5M solution of LiHMDS in THF (7.0 mL, 10.6 mmol) was slowly added and the reaction mixture was stirred at room temperature for 1 h. Water was added, and the reaction mixture was extracted with EtOAc (3×). The combined organic layers were washed with brine, dried over Na₂SO₄, filtered and concentrated to dryness. The crude solid was purified by column chromatography on silica gel (using a gradient of 0% to 50% EtOAc in DCM as eluent) to afford the two separated title products: 3-fluoro-4-methoxy-10-methyl-5,8,12-triazatricyclo[9.4.0.0²,7]pentadeca-1(11),2,4,6,12,14-hexaen-9-one N59a as a beige solid (289 mg, yield: 29%). LC-MS (Method B1) m/z [M+H]⁺: 274; rt: 1.05 min; purity: 99%. ¹H NMR (400 MHz, DMSO-d₆) δ 10.29 (s, 1H), 8.71 (dd, J=4.8, 1.7 Hz, 1H), 8.15 (ddd, J=7.9, 4.0, 1.7 Hz, 1H), 7.97 (s, 1H), 7.50 (dd, J=7.9, 4.8 Hz, 1H), 4.00 (s, 3H), 3.65 (q, J=6.6 Hz, 1H), 1.46 (d, J=6.6 Hz, 3H). ¹⁹F NMR (376 MHz, DMSO-d₆) δ−145.23 (d, J=4.0 Hz). 5-fluoro-4-methoxy-10-methyl-3,8,12-triazatricyclo[9.4.0.0²,7]pentadeca-1(11),2,4,6,12,14-hexaen-9-one N59b as a beige solid (272 mg, yield: 25%). LC-MS (Method B1) m/z [M+H]⁺: 274; rt: 1.12 min; purity: 98%. ¹H NMR (400 MHz, DMSO-d₆) δ 10.26 (s, 1H), 8.69 (dd, J=4.7, 1.8 Hz, 1H), 8.29 (dd, J=7.8, 1.8 Hz, 1H), 7.57-7.44 (m, 2H), 4.05 (s, 3H), 3.49 (q, J=6.6 Hz, 1H), 1.49 (d, J=6.6 Hz, 3H). ¹⁹F NMR (376 MHz, DMSO-d₆) δ−139.08 (d, J=10.8 Hz).

### Intermediate N60: 3-chloro-10-fluoro-5-methyl-4,8,12-triazatricyclo[9.4.0.0^{2,7}]pentadeca-1(11),2(7),3,5,12,14-hexaen-9-one

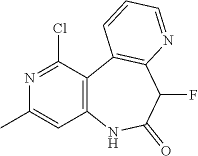 [SMILES: Cc1cc2c(c(Cl)n1)-c1cccnc1C(F)C(=O)N2]

### Step 1: Synthesis of ethyl 2-[3-(4-amino-2-chloro-6-methyl-3-pyridyl)-2-pyridyl]acetate N60_1

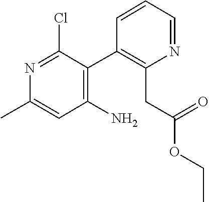 [SMILES: CCOC(=O)Cc1ncccc1-c1c(N)cc(C)nc1Cl]

A suspension of 2-chloro-3-iodo-6-methyl-pyridin-4-amine (N19_1) (2.0 g, 7.2 mmol), ethyl 2-[3-(4,4,5,5-tetramethyl-1,3,2-dioxaborolan-2-yl)-2-pyridyl]acetate (Intermediate N25_2, 4.2 g, 14.4 mmol), bis(di-tert-butyl(4-dimethylaminophenyl)phosphine)dichloropalladium(II) (530 mg, 0.71 mmol) and K₃PO₄ (3.0 g, 21 mmol) in 1,4-Dioxane (20 mL) and water (4 mL) was degassed with argon for 5 min. The reaction mixture was then heated at 80° C. for 3 h. After cooling to room temperature, EtOAc (20 mL) and water (10 mL) were added and the aqueous layer was extracted with EtOAc. The combined organic extracts were dried over MgSO₄, filtered and concentrated under vacuum. The black residue was purified by column chromatography on silica gel (using a gradient of 20% to 100% EtOAc in heptane as eluent) to afford the title compound as an orange oil (1.5 g, yield: 42%). LC-MS (Method A1) m/z: [M+H]⁺: 306.0, rt: 0.69 min, purity: 96%. ¹H NMR (400 MHz, CDCl₃) δ 8.67 (dd, J=4.8, 1.8 Hz, 1H), 7.55 (dd, J=7.7, 1.8 Hz, 1H), 7.38-7.32 (m, 1H), 6.45 (s, 1H), 4.14-4.04 (m, 2H), 3.75 (q, J=7.2 Hz, 2H), 2.43 (s, 3H), 1.21 (t, J=7.2 Hz, 3H).

### Step 2: Synthesis of ethyl 2-[3-(4-amino-2-chloro-6-methyl-3-pyridyl)-2-pyridyl]-2-fluoro-acetate N60_2

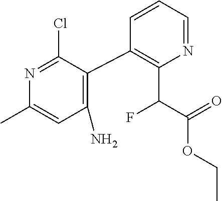 [SMILES: CCOC(=O)C(F)c1ncccc1-c1c(N)cc(C)nc1Cl]

To a solution of ethyl 2-[3-(4-amino-2-chloro-6-methyl-3-pyridyl)-2-pyridyl]acetate (Intermediate N60_1, 415 mg, 0.81 mmol) in ACN (2.0 mL) and water (2.0 mL) was added 1-chloromethyl-4-fluoro-1,4-diazoniabicyclo[2.2.2]octane bis(tetrafluoroborate) (560 mg, 1.5 mmol) and the reaction mixture was stirred at room temperature for 10 min. The reaction mixture was concentrated under vacuum and the residue was treated with cold water and extracted with DCM (3×10 mL). The combined organic layers were dried over MgSO₄, filtered off and concentrated under vacuum to give the title product (250 mg, yield: 75%) as a crude brown oil. The product was taken to the next step without purification LC-MS (Method A1) m/z [M+H]⁺: 324.0, rt: 0.76 and 0.78 min, purity: 75%.

### Step 3: Synthesis of 3-chloro-10-fluoro-5-methyl-4,8,12-triazatricyclo[9.4.0.0^{2,7}]pentadeca-1(11),2(7),3,5,12,14-hexaen-9-one N60

A suspension of ethyl 2-[3-(4-amino-2-chloro-6-methyl-3-pyridyl)-2-pyridyl]-2-fluoro-acetate (Intermediate N60_2, 100 mg, 0.30 mmol) and K₂CO₃ (86 mg, 0.60 mmol) in EtOH (1 mL) was stirred at room temperature for 20 h. The resulting white precipitate was collected by filtration to give the title product (105 mg, yield: 41%) as a white solid. LC-MS (Method A1) m/z [M+H]⁺: 278.1, rt: 0.87 min, purity: 96%. ¹H NMR (400 MHz, DMSO-d₆) δ 8.52 (td, J=4.6, 1.6 Hz, 1H), 8.14 (dt, J=7.9, 1.6 Hz, 1H), 7.32 (dd, J=7.9, 4.7 Hz, 1H), 6.56 (d, J=0.7 Hz, 1H), 5.37-5.09 (d, J=127, 1H), 2.30 (s, 3H).

### Intermediate N61: (10R)-3-fluoro-5,10-dimethyl-4,8,12-triazatricyclo[9.4.0.0²,7]pentadeca-1(11),2(7),3,5,12,14-hexaen-9-one

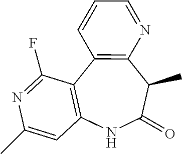

### Step 1: Synthesis of ethyl (2R)-2-(3-bromo-2-pyridyl)propanoate N61_1

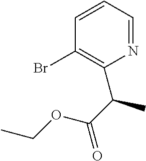

Ethyl 2-(3-bromo-2-pyridyl)propanoate was purified by chiral HPLC SFC (column chiralpak IC from Daicel, using EtOH:CO2 (98:2). Chiral purity>99%; tr=4.04 min (measured by HPLC, chiralpak IC from Daicel, using i-PrOH 10%—heptane 90%—DEA 0.1%, Temp: 30° C.). Second eluting isomer.

### Step 2: Synthesis of (10R)-3-fluoro-5,10-dimethyl-4,8,12-triazatricyclo[9.4.0.0^{2,7}]pentadeca-1(11),2(7),3,5,12,14-hexaen-9-one N61

The title product was synthesized following the same procedure as for intermediate N23, starting from (2R)-2-(3-bromo-2-pyridyl)propanoate N61_1. Chiral purity: 98%; rt=1.99 min (measured by HPLC, chiralpak IG-u from Daicel, EtOH 50%—heptane 50%—DEA 0.1%, Temp: 30° C.). First eluting peak. LC-MS (Method A1) m/z: [M+H]⁺: 258.0; rt: 0.93 min; purity: 94%. ¹H NMR (400 MHz, DMSO-d₆) δ 10.80 (s, 1H), 8.66 (dd, J=4.8, 1.8 Hz, 1H), 8.08 (ddt, J=6.3, 4.8, 1.8 Hz, 1H), 7.46 (dd, J=7.9, 4.8 Hz, 1H), 7.00 (s, 1H), 3.61 (q, J=6.5 Hz, 1H), 2.45 (s, 3H), 1.49 (d, J=6.5 Hz, 3H).

### Intermediate N62: 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid

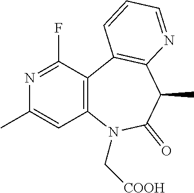

The title product was synthesized following the same procedure as for example #128, (steps 1 and 2), starting from intermediate N61. Chiral purity: >99%; rt=1.59 min (measured by HPLC, chiralpak AD from Daicel, EtOH 30%—heptane 70%—DEA 0.1%, Temp: 30° C.).

### Intermediate N63: 5,10-dimethyl-9-oxo-4,8,12-triazatricyclo[9.4.0.0^{2,7}]pentadeca-1(11),2(7),3,5,12,14-hexaene-3-carbonitrile

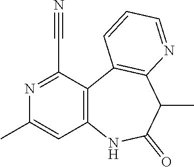

A stirred mixture of 4-amino-3-bromo-6-methyl-pyridine-2-carbonitrile (Intermediate N52, 500 mg, 2.24 mmol), ethyl 2-[3-(4,4,5,5-tetramethyl-1,3,2-dioxaborolan-2-yl)-2-pyridyl]propanoate (Intermediate N31_1, 1.37 g, 3.14 mmol) and CsF (953 mg, 6.27 mmol) in dry 1,4-dioxane (44 mL) and water (2.2 mL) was purged with nitrogen for 10 min. Bis(di-tert-butyl(4-dimethylaminophenyl)phosphine)dichloropalladium(II) (79 mg, 0.112 mmol) was added, the reaction mixture was purged with nitrogen for a further 5 min and then stirred at reflux for 18 h. The reaction mixture was allowed to cool to room temperature, then filtered through Celite® and washed through with ethyl acetate (100 mL). The filtrate was concentrated under vacuum. The residue was taken up in absolute ethanol (21 mL), to which potassium carbonate (619 mg, 4.48 mmol) was added and the reaction mixture was heated at 85° C. for 4 h. The reaction mixture was concentrated under vacuum, then treated with water (75 mL) and extracted with ethyl acetate (3×75 mL). The combined organic extracts were dried over sodium sulfate, filtered and concentrated under vacuum. The residue was purified by column chromatography on silica gel (using 0-100% ethyl acetate in iso-hexane as eluent) to afford the title compound (115 mg, yield: 19%) as an off-white solid. LC-MS (Method A7) m/z [M+H]⁺: 265; rt: 1.36 min; purity>99%.

### Intermediate N64: 1-methoxy-3,7-dimethyl-5,7-dihydropyrido[4,3-d][3]benzazepin-6-one

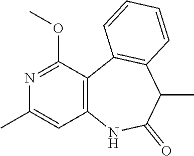

### Step 1: Synthesis of ethyl 2-(2-bromophenyl)propanoate N64_1

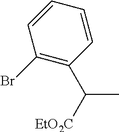

At 0° C., to a solution of ethyl 2-(2-bromophenyl)acetate (CAS 2178-24-7, 5.00 g, 20.6 mmol) in dry THE (50.0 mL) was added a 1 M solution of lithium bis(trimethylsilyl)amide in THF (22.6 mL, 22.6 mmol) and iodomethane, The reaction mixture was stirred at 0° C. for 1 h then at room temperature for 3 h. The reaction mixture was diluted with saturated aqueous ammonium chloride (100 mL) and extracted with EtOAc (3×100 mL). The organic layers were combined, washed with saturated aqueous ammonium chloride (150 mL), brine (100 mL) then dried over sodium sulfate, filtered and concentrated under vacuum. The residue was purified by flash chromatography on silica gel (using a gradient of 0 to 50% EtOAc in iso-hexane as eluent) to give the title compound (4.39 g, yield: 82%) as a colorless oil. LC-MS (Method A7) m/z: [M+H]⁺: 257/259; rt: 2.40 min; purity: 99%. ¹H NMR (400 MHz, DMSO-d₆) δ 7.62 (dd, J=8.0, 1.3 Hz, 1H), 7.42-7.31 (m, 2H), 7.27-7.17 (m, 1H), 4.19-3.97 (m, 3H), 1.40 (d, J=7.2 Hz, 3H), 1.13 (t, J=7.1 Hz, 3H).

### Step 2: Synthesis of ethyl 2-[2-(4,4,5,5-tetramethyl-1,3,2-dioxaborolan-2-yl)phenyl]propanoate N64_2

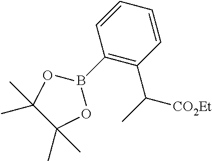 [SMILES: CCOC(=O)C(C)c1ccccc1B1OC(C)(C)C(C)(C)O1]

To a suspension of ethyl 2-(2-bromophenyl)propanoate (Intermediate N64_1, 2.00 g, 7.78 mmol), bis(pinacolato)diboron (2.37 mg, 9.33 mmol), potassium acetate (3.05 g, 31.1 mmol) in dry 1,4-dioxane (40 mL) under a nitrogen atmosphere was added [1,1′-bis(diphenylphosphino)ferrocene]-dichloropalladium(II) (285 mg, 0.389 mmol). The suspension was heated at reflux overnight. The reaction mixture was filtered through a pad of Celite® and washed with EtOAc (250 mL). The filtrate was concentrated under reduced pressure. The residue was purified by flash chromatography on silica gel (using a gradient of 0 to 30% EtOAc in iso-hexane as eluent) to give the title compound (1.1 g, yield: 44%) as a colorless oil. ¹H NMR (400 MHz, CDCl₃) δ 7.80 (dd, J=7.6, 1.6 Hz, 1H), 7.39 (td, J=7.6, 1.6 Hz, 1H), 7.29 (dd, J=7.9, 1.2 Hz, 1H), 7.23 (td, J=7.4, 1.2 Hz, 1H), 4.65 (q, J=7.1 Hz, 1H), 4.17-4.02 (m, 2H), 1.47 (d, J=7.1 Hz, 3H), 1.35 (d, J=2.3 Hz, 12H), 1.18 (t, J=7.1 Hz, 3H).

### Step 3: Synthesis of 3-bromo-2-methoxy-6-methylpyridin-4-amine N64_3

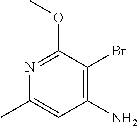

To a solution of 2-methoxy-6-methyl-pyridin-4-amine (5.00 g, 36.2 mmol) in DCM (120 mL) at 0° C. was added N-bromosuccinimide (6.76 g, 38.0 mmol) portionwise over 30 min. The reaction mixture was stirred at 0° C. for 1 h then water (150 mL) was added. The phases were separated, then the aqueous layer was extracted with DCM (2×80 mL). The organic layers were combined, dried over Na₂SO₄, filtered, and concentrated under vacuum. The residue was purified by flash chromatography on silica gel (using a gradient of 0 to 2% (0.7 M NH₃/MeOH) in DCM as eluent) to afford the title compound (6.75 g, yield: 85%) as an off white solid. LC-MS (Method B5′) m/z: [M+H]⁺: 217/219; rt: 1.57 min; purity: 99%. ¹H NMR (400 MHz, CDCl₃) δ 6.14 (d, J=3.1 Hz, 1H), 4.47 (s, 2H), 3.95 (s, 3H), 2.29 (d, J=2.1 Hz, 3H).

### Step 4: Synthesis of ethyl 2-[2-(4-amino-2-methoxy-6-methyl-3-pyridyl)phenyl]propanoate N64_4

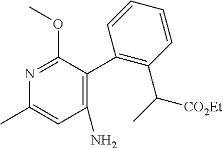 [SMILES: CCOC(=O)C(C)c1ccccc1-c1c(N)cc(C)nc1OC]

To a stirred solution of ethyl 2-[2-(4,4,5,5-tetramethyl-1,3,2-dioxaborolan-2-yl)phenyl]propanoate (Intermediate N64_2, 800 mg, 2.37 mmol) in 1,4-dioxane (15 mL) and water (1.5 mL) was added 3-bromo-2-methoxy-6-methylpyridin-4-amine (Intermediate N64_3, 565 mg, 2.60 mmol), SPhos Pd(crotyl)Cl (CAS: 1798781-99-3, 144 mg, 0.237 mmol) and cesium fluoride (1.08 g, 7.10 mmol). The reaction mixture was stirred at 90° C. for 18 hours. The reaction mixture was concentrated to dryness and the residue was purified by flash chromatography on silica gel (using a gradient of 0 to 2% (0.7 M NH₃/MeOH) in DCM as eluent) to afford the title compound (274 mg, yield: 29%) as a pale brown oil. LC-MS (Method B5′) m/z: [M+H]⁺: 315; rt: 1.18 min; purity: 80%.

### Step 5: Synthesis of 1-methoxy-3,7-dimethyl-5,7-dihydropyrido[4,3-d][3]benzazepin-6-one N64

To a stirred a solution of ethyl 2-[2-(4-amino-2-methoxy-6-methyl-3-pyridyl)phenyl]propanoate (Intermediate 64_4, 270 mg, 0.687 mmol) in EtOH (10.0 mL) was added K₂CO₃ (285 mg, 2.06 mmol) and the resulting suspension was stirred at 90° C. for 18 hours. The volatiles were removed under vacuum and the residue was treated with water (30 mL) and extracted with EtOAc (2×20 mL). The combined organic layers were dried over anhydrous Na₂SO₄, filtered and concentrated under reduced pressure to afford the title compound (169 mg, yield: 78%) as an off white solid. LC-MS (Method B5′) m/z: [M+H]⁺: 269; rt: 1.99 min; purity: 86%.

### Intermediate N65: 3,12-difluoro-5-methyl-4,8,14-triazatricyclo[9.4.0.0^{2,7}]pentadeca-1(11),2(7),3,5,12,14-hexaen-9-one

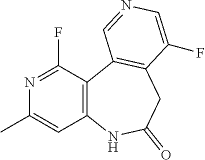 [SMILES: Cc1cc2c(c(F)n1)-c1cncc(F)c1CC(=O)N2]

### Step 1: Synthesis of ethyl 2-(3-bromo-5-fluoro-4-pyridyl)acetate N65_1

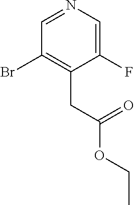

A 1 M solution of LiHMDS in THF (15.8 mL, 15.8 mmol) diluted in dry THF (7 mL) was added dropwise over 20 min to 3-bromo-5-fluoro-4-methyl-pyridine (1.0 g, 5.26 mmol) at room temperature. Upon completion of addition, the reaction mixture was stirred at room temperature for 1 h then neat diethyl carbonate (0.76 mL, 6.32 mmol) was added dropwise over 5 min. The reaction mixture was stirred at room temperature for 1 h, before being carefully neutralized by the addition of saturated ammonium chloride (10 mL). The reaction mixture was extracted with EtOAc (2×20 mL), the organic layers were combined, dried over Na₂SO₄, filtered and concentrated under vacuum. The product was purified by flash chromatography on silica gel (using a gradient of 0 to 30% MTBE in iso-hexane as eluent) to give the title compound (1.01 g, yield: 71%) as a colourless oil. LC-MS (Method A7) m/z: [M+H]⁺: 262/264; rt: 1.79 min; purity: 98%. ¹H NMR (400 MHz, CDCl₃) δ 8.56 (s, 1H), 8.40 (s, 1H), 4.20 (q, J=7.1 Hz, 2H), 3.87 (d, J=1.6 Hz, 2H), 1.27 (t, J=7.1 Hz, 3H).

### Step 2: Synthesis of ethyl 2-[3-(4-amino-2-fluoro-6-methyl-3-pyridyl)-5-fluoro-4-pyridyl]acetate N65_2

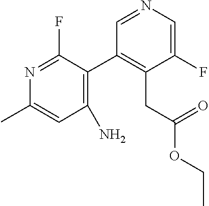 [SMILES: CCOC(=O)Cc1c(F)cncc1-c1c(N)cc(C)nc1F]

A solution of ethyl 2-(3-bromo-5-fluoro-4-pyridyl)acetate (Intermediate N65_1, 80 mg, 0.305 mmol), 2-fluoro-6-methyl-3-(4,4,5,5-tetramethyl-1,3,2-dioxaborolan-2-yl)pyridin-4-amine (Intermediate N16_1, 117 mg, 0.458 mmol) and cesium fluoride (139 mg, 0.916 mmol) in 1,4-dioxane (2.7 mL) and water (0.3 mL) was degassed with nitrogen for 5 min then bis(di-tert-butyl(4-dimethylaminophenyl)phosphine)dichloropalladium(II) (11 mg, 0.015 mmol) was added. The suspension was heated at reflux overnight then cooled to room temperature. The suspension was filtered through a pad of Celite, washed with EtOAc (50 mL) and the filtrate was concentrated to dryness. The residue was purified by flash chromatography on silica gel (using a gradient of 0 to 100% EtOAc in iso-hexane as eluent) to give the title compound (74 mg, yield: 74%) as a beige solid. LC-MS (Method A7) m/z: [M+H]⁺: 308; rt: 1.47 min; purity: 94%.

### Step 3: Synthesis of 3,12-difluoro-5-methyl-4,8,14-triazatricyclo[9.4.0.02 ⁷]pentadeca-1(11),2(7),3,5,12,14-hexaen-9-one N65

To a solution of ethyl 2-[3-(4-amino-2-fluoro-6-methyl-3-pyridyl)-5-fluoro-4-pyridyl]acetate (Intermediate N65_2, 74 mg, 0.241 mmol) in dry THF (2.0 mL) was added dropwise a 1 M solution of lithium bis(trimethylsilyl)amide in MTBE (0.25 mL, 0.253 mmol) and the reaction mixture was stirred at room temperature for 4 h. Water (5 mL) was added and the mixture was extracted with EtOAc (3×10 mL). The combined organic extracts were washed with brine, dried over Na₂SO₄, filtered and concentrated under vacuum to give the title compound (63 mg, yield: 96%) as a white solid. LC-MS (Method A7) m/z: [M+H]⁺: 262; rt: 1.27 min; purity: 99%. ¹H NMR (400 MHz, DMSO-d₆) δ 10.89 (s, 1H), 8.73 (d, J=5.3 Hz, 1H), 8.67 (s, 1H), 7.01 (s, 1H), 3.79 (d, J=13.0 Hz, 1H), 3.51 (d, J=13.0 Hz, 1H), 2.46 (s, 3H).

### Intermediate N66: 2-[2-(methylamino)-5-oxo-6H-thiazolo[4,5-d][3]benzazepin-4-yl]-N-[4-(trifluoromethyl)phenyl]acetamide

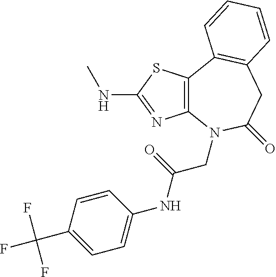 [SMILES: CNc1nc2c(s1)-c1ccccc1CC(=O)N2CC(=O)Nc1ccc(C(F)(F)F)cc1]

### Step 1: Synthesis of methyl 4-(tert-butoxycarbonylamino)-2-methylsulfanyl-thiazole-5-carboxylate

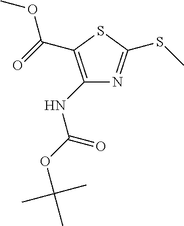

At 0° C., to a solution of 4-amino-2-methylthio-thiazole-5-carboxylic acid methyl ester (4.0 g, 19 mmol) in dry THF (56 mL) was slowly added sodium hydride (60% dispersion in mineral oil, 1.5 g, 41 mmol), followed by a solution of di-tert-butyl-dicarbonate (5.0 g, 22 mmol) in dry THF (30 mL) and the reaction mixture was stirred at room temperature for 45 min. The reaction mixture was diluted with EtOAc (250 mL) and washed with saturated NH₄Cl (3×50 mL). The organic layer was separated, dried over MgSO₄, filtered off and concentrated under vacuum. Purification by flash chromatography on silica gel (using a gradient of hept/EtOAc 100:0 to 70:30 as eluent) afforded methyl 4-(tert-butoxycarbonylamino)-2-methylsulfanyl-thiazole-5-carboxylate (5.56 g, yield: 97%) as a white crystalline solid. LC-MS (Method-A1) m/z: [M+H]⁺: 205/249, rt: 1.51 min, purity: 99%. ¹H NMR (400 MHz, CDCl₃) δ 9.18 (s, 1H), 3.85 (s, 3H), 2.71 (s, 3H), 1.53 (s, 9H).

### Step 2: Synthesis of 4-(tert-butoxycarbonylamino)-2-methylsulfanyl-thiazole-5-carboxylic acid N66_2

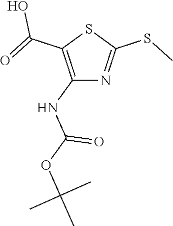 [SMILES: CSc1nc(NC(=O)OC(C)(C)C)c(C(=O)O)s1]

To a solution of methyl 4-(tert-butoxycarbonylamino)-2-methylsulfanyl-thiazole-5-carboxylate (Intermediate N66_1, 5.56 g, 18.3 mmol) in THF (91 mL) was added a 2 M aqueous solution of lithium hydroxide (18 mL, 36 mmol) and the reaction mixture was stirred at 50° C. for 18 h. THF was removed under vacuum and the resulting aqueous layer was acidified with a 1 N aqueous solution of HCl to pH=1. The resulting precipitate was collected by filtration and dried under vacuum to give 4-(tert-butoxycarbonylamino)-2-methylsulfanyl-thiazole-5-carboxylic acid (5.05 g, yield: 95%) as a white solid. LC-MS (Method-A1) m/z: [M+H]⁺: 191/235, rt: 1.21 min, purity: 99%. ¹H NMR (400 MHz, DMSO-d₆) δ 9.45 (s, 1H), 2.70 (s, 3H), 1.45 (s, 9H).

### Step 3: Synthesis of tert-butyl N-(5-bromo-2-methylsulfanyl-thiazol-4-yl)carbamate N66_3

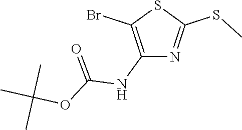 [SMILES: CSc1nc(NC(=O)OC(C)(C)C)c(Br)s1]

A suspension of 4-(tert-butoxycarbonylamino)-2-methylsulfanyl-thiazole-5-carboxylic acid (Intermediate N66_2, 1.0 g, 3.4 mmol), tetra-n-butylammonium tribromide (1.7 g, 6.9 mmol) and K₃PO₄ (750 mg, 3.4 mmol) in dry acetonitrile (17 mL) was stirred at 50° C. for 2 h. After cooling to room temperature, the reaction mixture was treated with saturated Na₂S₂O₃ and saturated NaHCO₃ solutions and extracted with dichloromethane (3×30 mL). The combined organic layers were washed with brine, dried over MgSO₄, filtered off and concentrated to dryness. Purification by flash chromatography on silica gel (using a gradient of heptane/EtOAc from 100:0 to 70:30 as eluent) afforded tert-butyl N-(5-bromo-2-methylsulfanyl-thiazol-4-yl)carbamate (0.98 g, yield: 88%) as an orange solid. LC-MS (Method-A1) m/z: [M+H]⁺: 227/271, rt: 1.41 min, purity>99%. ¹H NMR (400 MHz, DMSO-d₆) δ 9.13 (s, 1H), 2.65 (s, 3H), 1.43 (s, 9H).

### Step 4: Synthesis of methyl 2-[2-[4-(tert-butoxycarbonylamino)-2-methylsulfanyl-thiazol-5-yl]phenyl]acetate N66_4

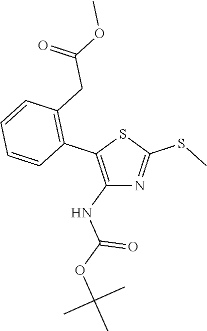 [SMILES: COC(=O)Cc1ccccc1-c1sc(SC)nc1NC(=O)OC(C)(C)C]

Under inert atmosphere, to a suspension of tert-butyl N-(5-bromo-2-methylsulfanyl-thiazol-4-yl)carbamate (Intermediate N66_3, 1.0 g, 3.1 mmol), methyl 2-(2-(4,4,5,5-tetramethyl-1,3,2-dioxaborolan-2-yl)phenyl)acetate (1.3 g, 4.6 mmol) and K₃PO₄ (1.3 g, 5.9 mmol) in 1,4-dioxane (28 mL) and water (3 mL) was added bis(di-tert-butyl(4-dimethylaminophenyl)phosphine)dichloropalladium(II) (220 mg, 0.31 mmol) and the reaction mixture was stirred at 100° C. for 6 h in sealed reactor. After cooling to room temperature, the reaction mixture was filtered through a pad of celite, rinsed with EtOAc, and the filtrate was concentrated under vacuum. Purification by flash chromatography on silica gel (using a gradient of Heptane/EtOAc from 100:0 to 70:30 as eluent) afforded the title product (813 mg, yield: 66%) as an orange oil. LC-MS (Method-B1) m/z: [M+H]⁺: 295/339, rt: 1.50 min, purity: 99%. ¹H NMR (400 MHz, DMSO-d₆) δ 8.85 (s, 1H), 7.41-7.30 (m, 3H), 7.26 (d, J=7.4 Hz, 1H), 3.65 (s, 2H), 3.57 (s, 3H), 2.67 (s, 3H), 1.24 (s, 9H).

### Step 5: Synthesis of 2-methylsulfanyl-4,6-dihydrothiazolo[4,5-d][3]benzazepin-5-one N66_5

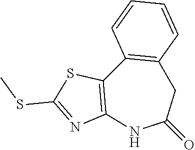 [SMILES: CSc1nc2c(s1)-c1ccccc1CC(=O)N2]

To a solution of methyl 2-[2-[4-(tert-butoxycarbonylamino)-2-methylsulfanyl-thiazol-5-yl]phenyl]acetate (Intermediate N66_4, 810 mg, 2.03 mmol) in dry dichloromethane (20 mL) was added a 4 N solution of HCl in dioxane (1.5 mL, 6.0 mmol) and the reaction mixture was stirred at room temperature for 18 h. The reaction mixture was evaporated to dryness to give the title product (528 mg, yield: 98%) as a light brown solid, which was taken crude to the next step. LC-MS (Method-A1) m/z: [M+H]⁺: 263.0, rt: 1.24 min, purity: 99%. ¹H NMR (400 MHz, DMSO-d₆) δ 10.85 (s, 1H), 7.41 (m, 4H), 3.52 (s, 2H), 2.74 (s, 3H).

### Step 6: Synthesis of 2-(2-methylsulfanyl-5-oxo-6H-thiazolo[4,5-d][3]benzazepin-4-yl)-N-[4-(trifluoromethyl)phenyl]acetamide N66_6

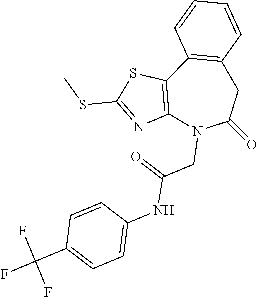 [SMILES: CSc1nc2c(s1)-c1ccccc1CC(=O)N2CC(=O)Nc1ccc(C(F)(F)F)cc1]

To a solution of 2-methylsulfanyl-4,6-dihydrothiazolo[4,5-d][3]benzazepin-5-one (Intermediate N66_5, 300 mg, 1.14 mmol) in dry DMF (6 mL) were added 2-chloro-N-[4-(trifluoromethyl)phenyl]acetamide (CAS 2707-23-5, 324 mg, 1.36 mmol), K₂CO₃ (474 mg, 3.43 mmol) and potassium iodide (19 mg, 0.11 mmol) and the reaction mixture was stirred at room temperature for 3 h. The reaction mixture was poured into cold water and the resulting precipitate was collected by filtration, rinsed with cold water and dried under vacuum to give the title product (543 mg, yield: 95%) as a brown solid. LC-MS (Method-A1) m/z: [M+H]⁺: 464.0, rt: 1.6 min, purity: 93%. ¹H NMR (400 MHz, DMSO-d₆) δ 10.50 (s, 1H), 7.73 (d, J=8.6 Hz, 2H), 7.64 (d, J=8.6 Hz, 2H), 7.53-7.38 (m, 4H), 4.72 (s, 2H), 3.70 (s, 2H), 2.68 (s, 3H)

### Step 7: Synthesis of 2-(2-methylsulfonyl-5-oxo-6H-thiazolo[4,5-d][3]benzazepin-4-yl)-N-[4-(trifluoromethyl)phenyl]acetamide N66_7

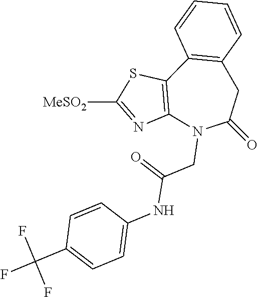

To a suspension of 2-(2-methylsulfanyl-5-oxo-6H-thiazolo[4,5-d][3]benzazepin-4-yl)-N-[4-(trifluoromethyl)phenyl]acetamide (Intermediate N66_6, 543 mg, 1.1 mmol) in dry dichloromethane (4 mL) and absolute ethanol (4 mL) was added ammonium molybdate (42 mg, 0.21 mmol). At 0° C., a 35% aqueous solution of H₂O₂ (1 mL, 11.6 mmol) was added dropwise and the reaction mixture was stirred at room temperature for 3 days. At 0 C°, a 35% aqueous solution of H₂O₂ (1 mL, 11.6 mmol) and ammonium molybdate (42 mg, 0.21 mmol) were added again and the reaction mixture was stirred at room temperature for additional 2 days. At 0° C., the reaction mixture was neutralized by addition of saturated Na₂S₂O₃ and stirred for 10 min, then water (5 mL) was added. The resulting precipitate was collected by filtration and dried under vacuum. Purification by flash chromatography on silica gel (using a gradient of heptane/EtOAc from 70:30 to 40:60 as eluent) afforded the title product (392 mg, yield: 67%) as an off-white solid. LC-MS (Method-A1) m/z: [M+H]⁺: 496.0, rt: 1.41 min, purity: 93%. ¹H NMR (400 MHz, DMSO-d₆) δ 10.56 (s, 1H), 7.72-7.70 (m, 3H), 7.64 (d, J=8.7 Hz, 2H), 7.59-7.47 (m, 3H), 4.79 (s, 2H), 3.79 (s, 2H), 3.50 (s, 3H).

### Step 8: Synthesis of 2-[2-(methylamino)-5-oxo-6H-thiazolo[4,5-d][3]benzazepin-4-yl]-N-[4-(trifluoromethyl)phenyl]acetamide N66

To a solution of 2-(2-methylsulfonyl-5-oxo-6H-thiazolo[4,5-d][3]benzazepin-4-yl)-N-[4-(trifluoromethyl)phenyl]acetamide (Intermediate N66_7, 200 mg, 0.40 mmol) in dry DMSO (4 mL) was added a 2 M solution of methylamine in THF (0.6 mL, 1 mmol) and the reaction mixture was stirred at room temperature for 18 h. The reaction mixture was poured into cold water (50 mL) and the resulting precipitate was collected by filtration, rinsed with water and dried under vacuum to give the title product (167 mg, yield: 83%) as an off-white solid. LC-MS (Method-A1) m/z: [M+H]⁺: 447.0, rt: 1.44 min, purity: 90%. ¹H NMR (400 MHz, DMSO-d₆) δ 10.44 (s, 1H), 8.00 (q, J=4.6 Hz, 1H), 7.74 (d, J=8.6 Hz, 2H), 7.64 (d, J=8.6 Hz, 2H), 7.44-7.21 (m, 4H), 4.63 (s, 2H), 3.63 (s, 2H), 2.80 (d, J=4.6 Hz, 3H).

### Intermediate N67: 2-(1,3,7-trimethyl-2,6-dioxo-pyrido[3,2-d][1,3]benzodiazepin-5-yl)acetic acid

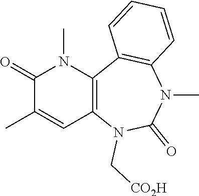 [SMILES: Cc1cc2c(n(C)c1=O)-c1ccccc1N(C)C(=O)N2CC(=O)O]

### Step 1: Synthesis of 6-methoxy-5-methyl-2-[2-(methylamino)phenyl]pyridin-3-amine N67_1

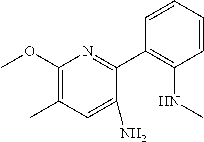

To a solution of N-methyl-2-(4,4,5,5-tetramethyl-1,3,2-dioxaborolan-2-yl)aniline (1.24 g, 5.05 mmol) in 1,4-dioxane (10 mL) and water (1.0 mL) were added 2-bromo-6-methoxy-5-methylpyridin-3-amine (Intermediate N68_1, 600 mg, 2.52 mmol), bis(di-tert-butyl(4-dimethylaminophenyl)phosphine) dichloropalladium(II) (179 mg, 0.252 mmol) and K₂CO₃ (1.05 g, 7.57 mmol) under inert atmosphere. The reaction mixture was then stirred at 80° C. for 16 h. The reaction mixture was concentrated under vacuum and purified by flash chromatography on silica gel (using a gradient of 0 to 100% EtOAc in iso-hexane as eluent) to afford the title compound (620 mg, yield: 85%) as a colorless oil. LC-MS (Method A7) m/z [M+H]⁺: 244.2; rt: 1.34 min, purity: 95%. ¹H NMR (400 MHz, DMSO-d₆) δ 7.19 (t, J=7.4 Hz, 2H), 7.06 (d, J=1.0 Hz, 1H), 6.67 (dd, J=8.0, 5.9 Hz, 2H), 5.25 (q, J=5.1 Hz, 1H), 4.30 (s, 2H), 3.74 (s, 3H), 2.71 (d, J=5.1 Hz, 3H), 2.09 (d, J=0.7 Hz, 3H).

### Step 2: Synthesis of 2-methoxy-3,7-dimethyl-5H-pyrido[3,2-d][1,3]benzodiazepin-6-one N67_2

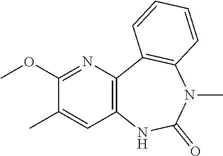

To a solution of 6-methoxy-5-methyl-2-[2-(methylamino)phenyl]pyridin-3-amine (Intermediate N67_1, 663 mg, 2.34 mmol) in DMF (8.0 mL) were added 1,1′-carbonyldiimidazole (493 mg, 3.04 mmol) and TEA (0.8 mL, 5.85 mmol) and the reaction mixture was heated at 50° C. for 16 h. The reaction mixture was then cooled to room temperature, diluted with water (20 mL) and extracted with EtOAc (3×20 mL). The combined organic extracts were washed with a 1M aqueous LiCl solution (20 mL) and brine (20 mL). The organic layer was then dried over MgSO₄, filtered and concentrated under vacuum. The residue was purified by flash chromatography on silica gel (using a gradient of 0 to 100% EtOAc in iso-hexane as eluent) to afford the title compound (640 mg, yield: 91%) as an off-white solid. LC-MS (Method A7) m/z [M+H]⁺: 270.2; rt: 2.12 min, purity: 95%. ¹H NMR (400 MHz, DMSO-d₆) δ 8.73 (s, 1H), 7.80 (dd, J=8.1, 1.7 Hz, 1H), 7.49-7.41 (m, 1H), 7.32-7.21 (m, 3H), 3.93 (s, 3H), 3.10 (s, 3H), 2.15 (s, 3H)

### Step 3: Synthesis of ethyl 2-(2-methoxy-3,7-dimethyl-6-oxo-pyrido[3,2-d][1,3]benzodiazepin-5-yl)acetate N67_3

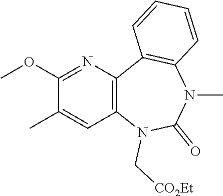

To a solution of 2-methoxy-3,7-dimethyl-5H-pyrido[3,2-d][1,3]benzodiazepin-6-one (Intermediate N67_2, 650 mg, 2.29 mmol) in DMF (10.0 mL) were added ethyl bromoacetate (0.25 mL, 2.29 mmol) and K₂CO₃ (634 mg, 4.59 mmol) and the reaction mixture was then heated at 60° C. for 16 h. Additional ethyl bromoacetate (0.25 mL, 2.29 mmol) was added and the reaction mixture was stirred at 60° C. for a further 3 h. The reaction mixture was then cooled to room temperature, treated with water (20 mL) and extracted with EtOAc (3×20 mL). The combined organic extracts were washed with a 1M aqueous LiCl solution (20 mL) and brine (20 mL), dried over MgSO₄, filtered and concentrated under vacuum. The residue was purified by flash chromatography on silica gel (using a gradient of 0 to 100% EtOAc in iso-hexane as eluent) to afford the title compound (270 mg, yield: 31%) as a colorless oil. LC-MS (Method A7) m/z [M+H]⁺: 356.2; rt: 2.43 min, purity: 99%. ¹H NMR (400 MHz, DMSO-d₆) δ 7.85 (dd, J=8.1, 1.6 Hz, 1H), 7.56-7.55 (m, 1H), 7.51-7.47 (m, 1H), 7.33-7.29 (m, 2H), 4.44 (d, J=17.1 Hz, 1H), 4.16 (d, J=17.1 Hz, 1H), 4.08-4.00 (m, 2H), 3.95 (s, 3H), 3.07 (s, 3H), 2.18 (s, 3H), 1.11 (d, J=7.1 Hz, 3H).

### Step 4: Synthesis of ethyl 2-(3,7-dimethyl-2,6-dioxo-1H-pyrido[3,2-d][1,3]benzodiazepin-5-yl)acetate N67_4

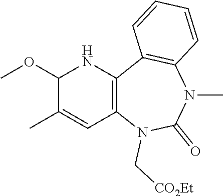

To a solution of ethyl 2-(2-methoxy-3,7-dimethyl-6-oxo-pyrido[3,2-d][1,3]benzodiazepin-5-yl)acetate (Intermediate N67_3, 260 mg, 0.695 mmol) in acetonitrile (8.0 mL) were added KI (577 mg, 3.48 mmol) and TMSCl (0.44 mL, 3.48 mmol) and the reaction mixture was heated at 80° C. for 2 h. The reaction mixture was concentrated under vacuum. The residue was then taken up in EtOAc (20 mL) and washed with water (20 mL) and saturated sodium thiosulfate (20 mL). The organic layer was dried over MgSO₄, filtered and concentrated under vacuum to afford the title compound (230 mg, yield: 92%) as a yellow solid. LC-MS (Method A7) m/z [M+H]⁺: 342.2; rt: 1.47 min; purity: 95%.

### Step 5: Synthesis of ethyl 2-(1,3,7-trimethyl-2,6-dioxo-pyrido[3,2-d][1,3]benzodiazepin-5-yl)acetate N67_5

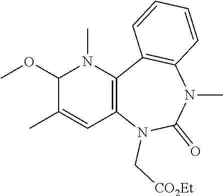

To a solution of ethyl 2-(3,7-dimethyl-2,6-dioxo-1H-pyrido[3,2-d][1,3]benzodiazepin-5-yl)acetate (Intermediate N67_4, 230 mg, 0.64 mmol) in acetonitrile (10.0 mL) were added cesium carbonate (459 mg, 1.41 mmol) and a 1M solution of iodomethane in acetonitrile (1.3 mL, 1.30 mmol) and the reaction mixture was then stirred at 80° C. for 16 h. The reaction mixture was concentrated under vacuum and the residue was purified by flash chromatography on silica gel (using a gradient of 0 to 10% MeOH in DCM as eluent) to afford the title compound (100 mg, yield: 37%) as a yellow solid. LC-MS (Method A7) m/z [M+H]⁺: 356.2; rt: 1.62 min; purity: 89%. ¹H NMR (400 MHz, DMSO-ds) δ 7.61 (dd, J=7.9, 1.5 Hz, 1H), 7.53 (td, J=7.8, 7.2, 1.5 Hz, 1H), 7.49 (d, J=1.3 Hz, 1H), 7.41 (d, J=8.2 Hz, 1H), 7.34-7.29 (m, 1H), 4.41 (d, J=17.1 Hz, 1H), 4.06 (d, J=17.1 Hz, 1H), 4.00-3.91 (m, 2H), 3.36 (s, 3H), 3.08 (s, 3H), 2.07 (d, J=1.1 Hz, 3H), 1.05 (t, J=7.1 Hz, 3H).

### Step 6: Synthesis of 2-(1,3,7-trimethyl-2,6-dioxo-pyrido[3,2-d][1,3]benzodiazepin-5-yl)acetic acid N67

To a solution of ethyl 2-(1,3,7-trimethyl-2,6-dioxo-pyrido[3,2-d][1,3]benzodiazepin-5-yl)acetate (Intermediate N67_5, 100 mg, 0.239 mmol) in MeOH (5.0 mL) and water (1.0 mL) was added LiOH—H₂O (25 mg, 0.598 mmol) and the reaction mixture was then stirred at room temperature for 16 h. The reaction mixture was then acidified with a 1N aq. solution of HCl (2.0 mL) and concentrated under vacuum to afford the title compound (80 mg, yield: 97%) as a yellow solid. LC-MS (Method A7) m/z [M+H]⁺: 328.2; rt: 2.23 min; purity: 95%.

### Intermediate N68: [2-(3,5,10-trimethyl-4,9-dioxo-3,8,12-triazatricyclo[9.4.0.0^{2,7}]pentadeca-1(11),2(7),5,12,14-pentaen-8-yl)acetyl]oxylithium

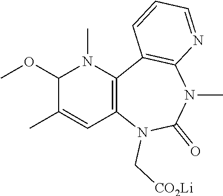

### Step 1: Synthesis of 2-bromo-6-methoxy-5-methylpyridin-3-amine N68_1

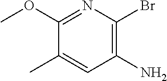

6-Methoxy-5-methyl-pyridin-3-amine (10.0 g, 72.4 mmol) was dissolved in acetic acid (100 mL) and sodium acetate (5.94 g, 72.4 mmol) was added. The reaction mixture was stirred at room temperature for 20 minutes. A solution of bromine (3.9 mL, 76.0 mmol) in acetic acid (50 mL) was added dropwise and the resulting brown solution was stirred at room temperature for 2 hours. The reaction mixture was slowly poured into a stirring solution of crushed ice and aqueous NaOH (2 N, 100 mL) and stirred at 0° C. for 30 minutes. The precipitate was collected by filtration then purified by flash chromatography on silica gel (using a gradient of 0 to 20% EtOAc in iso-hexane as eluent) to afford the title compound (14.9 g, yield: 89%) as a brown solid. LC-MS (Method A7) m/z: [M+H]⁺: 217/219; rt: 1.72 min; purity: 94%. ¹H NMR (400 MHz, DMSO-d₆) δ 7.03 (d, J=1.0 Hz, 1H), 4.81 (s, 2H), 3.74 (s, 3H), 2.02 (d, J=1.0 Hz, 3H).

### Step 2: Synthesis of ethyl 2-(3-bromo-2-pyridyl)propanoate N68_2

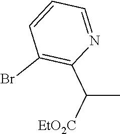

Ethyl 2-(3-bromo-2-pyridyl)acetate (Intermediate N3_1, 30.0 g, 117 mmol) was dissolved in dry THE (400 mL) and cooled to 0° C. A 1 M solution of lithium bis(trimethylsilyl)amide in THF (134 mL, 134 mmol) was added dropwise and stirred for 30 min. Iodomethane (21.5 g, 152 mmol) was added and the reaction mixture was stirred for a further 1 h at 0° C. The solution was concentrated under vacuum and the residue was purified by flash chromatography on silica gel (using a gradient of 0 to 60% MTBE in iso-hexane as eluent) to afford the title compound (29.5 g, yield: 96%) as a light-yellow oil. LC-MS (Method A7) m/z: [M+H]⁺: 258/260; rt: 1.93 min; purity: 99%. ¹H NMR (400 MHz, CDCl₃) δ 8.51 (dd, J=4.6, 1.5 Hz, 1H), 7.85 (dd, J=8.0, 1.5 Hz, 1H), 7.06 (dd, J=8.0, 4.6 Hz, 1H), 4.36 (q, J=7.1 Hz, 1H), 4.17 (q, J=7.1 Hz, 2H), 1.55 (d, J=7.1 Hz, 3H), 1.20 (t, J=7.1 Hz, 3H).

### Step 3: Synthesis of ethyl 2-[3-(3-amino-6-methoxy-5-methyl-2-pyridyl)-2-pyridyl]propanoate N68_3

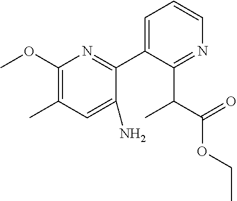 [SMILES: CCOC(=O)C(C)c1ncccc1-c1nc(OC)c(C)cc1N]

A stirring mixture of 2-bromo-6-methoxy-5-methylpyridin-3-amine (Intermediate N68_2, 2.00 g, 8.75 mmol), ethyl 2-[3-(4,4,5,5-tetramethyl-1,3,2-dioxaborolan-2-yl)-2-pyridyl]propanoate (Intermediate N31_1, 5.72 g, 13.1 mmol), bis(di-tert-butyl (4-dimethylaminophenyl)-phosphine)-dichloro palladium (II) (310 mg, 0.438 mmol) and cesium fluoride (3.99 g, 26.3 mmol) in 1,4-dioxane (40 mL) and water (3 mL) was degassed with nitrogen for 5 minutes and then heated at 90° C. for 3 hours. The reaction mixture was cooled to room temperature and filtered through a small pad of Celite. The filter cake was rinsed with EtOAc (80 mL) and the filtrate was concentrated under vacuum. The residue was purified by flash chromatography on silica gel (using a gradient of 0 to 100% EtOAc in iso-hexane as eluent) to afford the title compound (1.10 g, yield: 35%) as a thick brown oil. LC-MS (Method A7) m/z: [M+H]⁺: 316; rt: 1.70 min; purity: 95%. ¹H NMR (400 MHz, CDCl₃) δ 8.66 (dd, J=5.0, 1.8 Hz, 1H), 7.79 (d, J=7.7 Hz, 1H), 7.37-7.31 (m, 1H), 6.96 (d, J=1.0 Hz, 1H), 4.16-4.03 (m, 3H), 3.84 (s, 3H), 2.19 (d, J=0.9 Hz, 3H), 1.55 (d, J=7.2 Hz, 3H), 1.15 (t, J=7.1 Hz, 3H). protons for NH₂ not observed.

### Step 4: Synthesis of 4-methoxy-5,10-dimethyl-3,8,12-triazatricyclo[9.4.0.0^{2,7}]pentadeca-1(11),2(7),3,5,12,14-hexaen-9-one N68_4

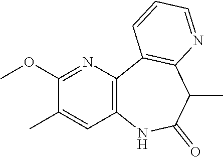

Ethyl 2-[3-(3-amino-6-methoxy-5-methyl-2-pyridyl)-2-pyridyl]propanoate (Intermediate N68_3, 511 mg, 1.30 mmol) was dissolved in EtOH (25 mL) and potassium carbonate (358 mg, 2.59 mmol) was added. The reaction mixture was stirred at 80° C. for 2 hours. The reaction mixture was concentrated under vacuum. The residue was suspended in water (50 mL) and acidified to pH ~5-7 with acetic acid (~5 mL). The aqueous suspension was extracted with EtOAc (4×50 mL) and the combined organic extracts were dried over sodium sulfate, filtered and concentrated under vacuum. The residue was triturated in iso-hexane (40 mL) and the solid was collected, washed with iso-hexane (30 mL) and dried under vacuum to afford the title compound (764 mg, yield: 99%) as a light beige solid. LC-MS (Method A7) m/z: [M+H]⁺: 270; rt: 1.72 min; purity: 98%. ¹H NMR (400 MHz, DMSO-d₆) δ 10.15 (s, 1H), 8.67 (dd, J=4.7, 1.8 Hz, 1H), 8.31 (dd, J=7.8, 1.8 Hz, 1H), 7.50 (dd, J=7.8, 4.7 Hz, 1H), 7.40 (d, J=1.0 Hz, 1H), 3.98 (s, 3H), 3.42 (s, 1H), 2.22 (d, J=1.0 Hz, 3H), 1.48 (d, J=6.6 Hz, 3H).

### Step 5: Synthesis of ethyl 2-(4-methoxy-5,10-dimethyl-9-oxo-3,8,12-triazatricyclo[9.4.0.0^{2,7}]pentadeca-1(11),2(7),3,5,12,14-hexaen-8-yl)acetate N68_5

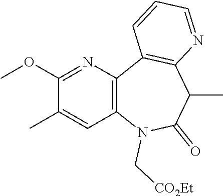

4-Methoxy-5,10-dimethyl-3,8,12-triazatricyclo[9.4.0.0^{2,7}]pentadeca-1(11),2(7),3,5,12,14-hexaen-9-one (Intermediate N68_4, 0.77 g, 2.72 mmol) was dissolved in dry DMF (30 mL) and potassium carbonate (1.13 g, 8.15 mmol) and ethyl 2-bromoacetate (635 mg, 3.80 mmol) were sequentially added. The reaction mixture was stirred at room temperature for 72 hours. The reaction mixture was diluted with water (50 mL) and extracted with EtOAc (2×50 mL). The combined organic extracts were washed with brine (2×50 mL), dried over sodium sulfate, filtered and concentrated under vacuum. The residue was purified by flash chromatography on silica gel (using a gradient of 0 to 100% EtOAc in iso-hexane as eluent) to afford the title compound (820 mg, yield: 77%) as a brown oil. LC-MS (Method A7) m/z: [M+H]⁺: 356; rt: 2.10 min; purity: 97%. ¹H NMR (400 MHz, CDCl₃) δ 8.72 (dd, J=4.8, 1.8 Hz, 1H), 8.32 (dd, J=7.8, 1.8 Hz, 1H), 7.43 (d, J=1.0 Hz, 1H), 7.41-7.36 (m, 1H), 4.44 (d, J=17.3 Hz, 1H), 4.24 (d, J=17.2 Hz, 1H), 4.17-4.10 (m, 2H), 4.05 (s, 3H), 3.64 (d, J=6.8 Hz, 1H), 2.28 (d, J=1.0 Hz, 3H), 1.71 (d, J=6.8 Hz, 3H), 1.16 (t, J=7.2 Hz, 3H).

### Step 6: Synthesis of ethyl 2-(5,10-dimethyl-4,9-dioxo-3,8,12-triazatricyclo[9.4.0.0^{2,7}]pentadeca-1(11),2(7),5,12,14-pentaen-8-yl)acetate N68_6

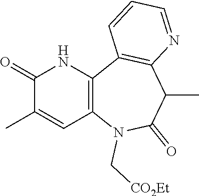

Ethyl 2-(4-methoxy-5,10-dimethyl-9-oxo-3,8,12-triazatricyclo[9.4.0.0^{2,7}]pentadeca-1(11),2(7),3,5,12,14-hexaen-8-yl)acetate (Intermediate N68_5, 1.00 g, 2.67 mmol) and potassium iodide (2.22 g, 13.4 mmol) were suspended in dry acetonitrile (40 mL) and trimethylsilyl chloride (1.70 mL, 13.4 mmol) was then added. The reaction mixture was stirred at 80° C. for 24 hours. Additional trimethylsilyl chloride (1.70 mL, 13.4 mmol) was added and the reaction mixture was stirred at 80° C. for further 24 hours. The reaction mixture was concentrated under vacuum and the residue was partitioned between 10% MeOH/DCM (50 mL) and water (30 mL). The layers were separated and the aqueous phase was extracted with 10% MeOH/DCM (3×100 mL). The combined organic extracts were washed with saturated aqueous sodium thiosulphate solution (40 mL), filtered through a hydrophobic frit and concentrated under vacuum. The residue was triturated in MTBE (25 mL), the solid collected, washed with MTBE (25 mL) and dried under vacuum to afford the title compound (853 mg, yield: 92%) as a white solid. LC-MS (Method A7) m/z: [M+H]⁺: 342; rt: 1.22 min; purity: 99%. ¹H NMR (400 MHz, DMSO-d₆) δ 8.67 (dd, J=4.8, 1.6 Hz, 1H), 8.10 (dd, J=7.9, 1.7 Hz, 1H), 7.58-7.54 (m, 1H), 7.48 (dd, J=7.9, 4.7 Hz, 1H), 4.46 (d, J=17.3 Hz, 1H), 4.27 (d, J=17.3 Hz, 1H), 3.92 (qq, J=6.9, 3.7 Hz, 2H), 3.71 (q, J=6.6 Hz, 1H), 2.10 (s, 3H), 1.48 (d, J=6.6 Hz, 3H), 0.97 (t, J=7.1 Hz, 3H). Proton for NH not observed.

### Step 7: Synthesis of ethyl 2-(3,5,10-trimethyl-4,9-dioxo-3,8,12-triazatricyclo[9.4.0.0^{2,7}]pentadeca-1(11),2(7),5,12,14-pentaen-8-yl)acetate N68_7

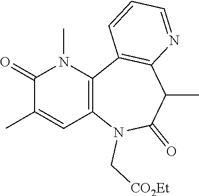

Ethyl 2-(5,10-dimethyl-4,9-dioxo-3,8,12-triazatricyclo[9.4.0.0^{2,7}]pentadeca-1(11),2(7),5,12,14-pentaen-8-yl)acetate (Intermediate N68 6. 0.85 g, 2.37 mmol) was dissolved in dry acetonitrile (40 mL) and cesium carbonate (2.31 g, 7.10 mmol) was added. After 10 minutes, iodomethane (672 mg, 4.73 mmol) was slowly added and the reaction mixture was stirred at room temperature for 3 hours. The reaction mixture was diluted with EtOAc (40 mL), filtered and concentrated under vacuum. The residue was purified by flash chromatography on silica gel (using a gradient of 0 to 6% MeOH in DCM as eluent) to afford the title compound (400 mg, yield: 45%) as a white foam. LC-MS (Method A7) m/z: [M+H]⁺: 356; rt: 1.33 min; purity: 99%. ¹H NMR (400 MHz, DMSO-d₆) δ 8.68 (dd, J=4.7, 1.7 Hz, 1H), 8.18 (dd, J=7.9, 1.7 Hz, 1H), 7.56 (d, J=1.3 Hz, 1H), 7.47 (dd, J=7.9, 4.7 Hz, 1H), 4.45 (d, J=17.3 Hz, 1H), 4.20 (d, J=17.3 Hz, 1H), 3.95-3.84 (m, 3H), 3.35 (s, 3H), 2.11 (d, J=1.1 Hz, 3H), 1.48 (d, J=6.7 Hz, 3H), 0.98 (t, J=7.1 Hz, 3H).

### Step 8: Synthesis of [2-(3,5,10-trimethyl-4,9-dioxo-3,8,12-triazatricyclo[9.4.0.0^{2,7}]pentadeca-1(11),2(7),5,12,14-pentaen-8-yl)acetyl]oxylithium N68

Ethyl 2-(3,5,10-trimethyl-4,9-dioxo-3,8,12-triazatricyclo[9.4.0.0^{2,7}]pentadeca-1(11),2(7),5,12,14-pentaen-8-yl)acetate (Intermediate N68_7, 220 mg, 0.600 mmol) was dissolved in THF (5 mL), MeOH (5 mL) and water (2.5 mL). Lithium hydroxide monohydride (50 mg, 1.20 mmol) was added and the reaction mixture was stirred at room temperature for 1 hour. The reaction mixture was concentrated to dryness to afford the title compound (200 mg, yield: 96%) as a white solid. The product was used in the next step without further purification. LC-MS (Method A7) m/z: [M+2H—Li]⁺: 328; rt: 1.04 min; purity: 97%.

### Intermediate N69: [2-(1,3,7-trimethyl-2,6-dioxo-7H-pyrido[3,2-d][3]benzazepin-5-yl)acetyl]oxylithium

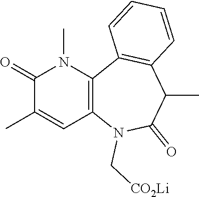

### Step 1: Synthesis of ethyl 2-[2-(3-amino-6-methoxy-5-methyl-2-pyridyl)phenyl]propanoate N69_1

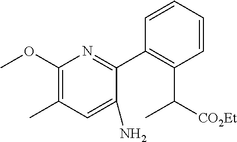 [SMILES: CCOC(=O)C(C)c1ccccc1-c1nc(OC)c(C)cc1N]

A stirred mixture of 2-bromo-6-methoxy-5-methyl-pyridin-3-amine (Intermediate N68_1, 1.70 g, 7.13 mmol), ethyl 2-[2-(4,4,5,5-tetramethyl-1,3,2-dioxaborolan-2-yl)phenyl]propanoate (Intermediate N64_2, 3.7 g, 10/7 mmol), bis(di-tert-butyl(4-dimethylaminophenyl)phosphine)dichloropalladium(II) (252 mg, 0.356 mmol) and CsF (3.25 g, 21.4 mmol) in 1,4-dioxane (40 mL) and water (3 mL) was degassed with nitrogen for 5 minutes and then heated at 90° C. for 3 h. The reaction mixture was allowed to cool to room temperature and filtered through a small pad of celite. The filter cake was rinsed with EtOAc (80 mL) and the filtrate was concentrated under vacuum. The residue was purified by flash chromatography on silica gel (using a gradient of 0 to 100% EtOAc in iso-hexane as eluent) to give the title compound (751 mg, yield: 33%) as a light green oil. LC-MS (Method A7) m/z [M+H]⁺: 315.2; rt: 2.12 min; purity: 99%.

### Step 2: Synthesis of 2-methoxy-3,7-dimethyl-5,7-dihydropyrido[3,2-d][3]benzazepin-6-one N69_2

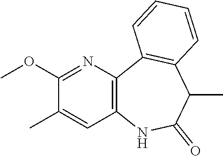

Ethyl 2-[2-(3-amino-6-methoxy-5-methyl-2-pyridyl)phenyl]propanoate (Intermediate N69_1, 0.751 g, 2.35 mmol) was dissolved in EtOH (50 mL) and K₂CO₃ (660 mg, 4.78 mmol) was added. The reaction mixture was stirred at 80° C. overnight, then evaporated to dryness and the residue was treated with water (100 mL). The resulting precipitate was collected by filtration, washed with water (30 mL) and dried under vacuum at 40° C. to give the title compound (618 mg, yield: 98%) as a light grey solid. LC-MS (Method A7) m/z [M+H]⁺: 269.2; rt: 2.17 min; purity: 99%. ¹H NMR (400 MHz, DMSO-d₆) δ 9.96 (s, 1H), 7.93 (dd, J=7.8, 1.6 Hz, 1H), 7.52 (td, J=7.6, 1.5 Hz, 1H), 7.44 (td, J=7.5, 1.4 Hz, 1H), 7.37-7.31 (m, 2H), 3.97 (s, 3H), 3.25 (d, J=6.9 Hz, 1H), 2.21 (d, J=0.9 Hz, 3H), 1.46 (d, J=6.9 Hz, 3H).

### Step 3: Synthesis of ethyl 2-(2-methoxy-3,7-dimethyl-6-oxo-7H-pyrido[3,2-d][3]benzazepin-5-yl)acetate N69_3

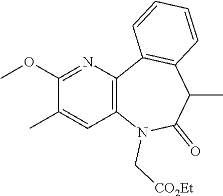

To a suspension of 2-methoxy-3,7-dimethyl-5,7-dihydropyrido[3,2-d][3]benzazepin-6-one (Intermediate N69_2, 500 mg, 1.83 mmol) in dry DMF (3 mL), potassium carbonate (505 mg, 3.65 mmol), KI (30 mg, 0.183 mmol) and ethyl 2-bromoacetate (0.25 mL, 2.19 mmol) were added sequentially and the reaction mixture was stirred at room temperature for 18 h. Water (100 mL) was added and the resulting solid was collected, washed with water (30 mL) and dried under vacuum at 40° C. to give the title compound (630 mg, yield: 95%). LC-MS (Method A7) m/z [M+H]⁺: 355.2; rt: 2.49 min; purity>99%. ¹H NMR (400 MHz, DMSO-d₆) δ 7.95 (dd, J=7.6, 1.6 Hz, 1H), 7.66 (d, J=1.0 Hz, 1H), 7.53 (td, J=7.5, 1.6 Hz, 1H), 7.46 (td, J=7.5, 1.3 Hz, 1H), 7.34 (d, J=7.7 Hz, 1H), 4.47-4.30 (m, 2H), 4.03-3.92 (m, 5H), 3.44 (q, J=7.1 Hz, 1H), 2.23 (d, J=1.0 Hz, 3H), 1.47 (d, J=6.8 Hz, 3H), 1.00 (t, J=7.1 Hz, 3H).

### Step 4: Synthesis of ethyl 2-(3,7-dimethyl-2,6-dioxo-1,7-dihydropyrido[3,2-d][3]benzazepin-5-yl)acetate N69_4

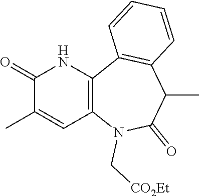

Ethyl 2-(2-methoxy-3,7-dimethyl-6-oxo-7H-pyrido[3,2-d][3]benzazepin-5-yl)acetate (Intermediate N69_3, 600 mg, 1.66 mmol) and KI (826 mg, 4.98 mmol) in dry MeCN (10.0 mL), TMSCl (0.63 mL, 4.98 mmol) was added and the reaction mixture was stirred at 80° C. for 3 h. The reaction mixture was concentrated under vacuum and the residue was partitioned between DCM (30 mL) and water (20 mL). The layers were separated, and the aqueous phase was extracted with DCM (3×40 mL). The combined organic extracts were washed with sat. sodium thiosulphate solution (10 mL), filtered through a hydrophobic frit and concentrated under vacuum to give the title compound (588 mg, quantitative yield) as a pale brown solid. LC-MS (Method A7) m/z [M+H]⁺: 341.2; rt: 1.49 min; purity: 98%. ¹H NMR (400 MHz, DMSO-d₆) δ 11.76 (s, 1H), 7.68 (d, J=7.7 Hz, 1H), 7.54 (td, J=7.6, 1.4 Hz, 1H), 7.50 (d, J=1.2 Hz, 1H), 7.43 (td, J=7.6, 1.2 Hz, 1H), 7.34 (d, J=7.8 Hz, 1H), 4.38 (d, J=17.3 Hz, 1H), 4.22 (d, J=17.3 Hz, 1H), 3.93 (qq, J=6.9, 3.7 Hz, 2H), 3.52 (q, J=6.8 Hz, 1H), 2.08 (d, J=1.1 Hz, 3H), 1.46 (d, J=6.8 Hz, 3H), 0.97 (t, J=7.1 Hz, 3H).

### Step 5: Synthesis of ethyl 2-(1,3,7-trimethyl-2,6-dioxo-7H-pyrido[3,2-d][3]benzazepin-5-yl)acetate N695

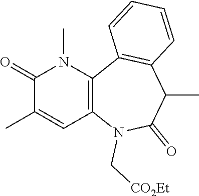

Ethyl 2-(3,7-dimethyl-2,6-dioxo-1,7-dihydropyrido[3,2-d][3]benzazepin-5-yl)acetate (Intermediate N69_4, 588 mg, 1.66 mmol) was dissolved in dry MeCN (20 mL) and cesium carbonate (1.62 g, 4.99 mmol) was added. After 10 minutes, iodomethane (472 mg, 3.32 mmol) was slowly added, and the reaction mixture was stirred at room temperature for 3 h. The reaction mixture was diluted with EtOAc (20 mL) and concentrated under vacuum in presence of silica. The residue was purified by flash chromatography on silica gel (using a gradient of 0 to 5% MeOH in DCM as eluent) to give the title compound (115 mg, yield: 19%). LC-MS (Method A7) m/z [M+H]⁺: 355.2; rt: 1.68 min; purity: 98%. ¹H NMR (400 MHz, DMSO-d₆) δ 7.67 (dd, J=7.7, 1.3 Hz, 1H), 7.55 (td, J=7.6, 1.4 Hz, 1H), 7.51 (d, J=1.3 Hz, 1H), 7.44-7.35 (m, 2H), 4.37 (d, J=17.3 Hz, 1H), 4.16 (d, J=17.3 Hz, 1H), 3.92 (qd, J=7.1, 5.0 Hz, 2H), 3.69 (q, J=6.8 Hz, 1H), 3.34 (s, 3H), 2.10 (d, J=1.1 Hz, 3H), 1.46 (d, J=6.8 Hz, 3H), 0.99 (t, J=7.1 Hz, 3H).

### Step 6: Synthesis of [2-(1,3,7-trimethyl-2,6-dioxo-7H-pyrido[3,2-d][3]benzazepin-5-yl)acetyl]oxylithium N69

Ethyl 2-(1,3,7-trimethyl-2,6-dioxo-7H-pyrido[3,2-d][3]benzazepin-5-yl)acetate (Intermediate N69_5, 115 mg, 0.316 mmol) was dissolved in THF (2.5 mL), MeOH (2.5 mL) and water (1.25 mL). LiOH·H₂O (27 mg, 0.633 mmol) was added, and the reaction mixture was stirred at room temperature for 1 h. The reaction mixture was concentrated to dryness and then co-evaporated with acetonitrile (3×10 mL). The solid residue was triturated with MTBE (5 mL) and dried to give the title compound (108 mg, quantitative yield) as a pale-yellow solid. LC-MS (Method A7) m/z [M−Li+2H]⁺: 327.2; rt: 1.39 min; purity: 98%. ¹H NMR (400 MHz, DMSO-d₆) δ 7.82 (d, J=1.3 Hz, 1H), 7.63 (dd, J=7.8, 1.4 Hz, 1H), 7.52 (td, J=7.6, 1.3 Hz, 1H), 7.40-7.32 (m, 2H), 3.91 (d, J=16.2 Hz, 1H), 3.53 (q, J=6.8 Hz, 1H), 3.20 (d, J=16.2 Hz, 1H), 2.06 (d, J=1.1 Hz, 3H), 1.45 (d, J=6.8 Hz, 3H). N—CH₃ obscured by the water peak.

### Intermediate N70: [2-(9-fluoro-2,3,7-trimethyl-1,6-dioxo-7H-pyrido[4,3-d][3]benzazepin-5-yl)acetyl]oxylithium

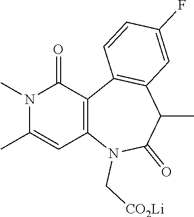

### Step 1: Synthesis of ethyl 2-(2-bromo-5-fluoro-phenyl)acetate N70_1

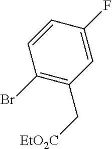 [SMILES: CCOC(=O)Cc1cc(F)ccc1Br]

To a stirred solution of 2-(2-bromo-5-fluoro-phenyl)acetic acid (10.0 g, 42.9 mmol) in EtOH (100 mL) was added concentrated H₂SO₄ (2.52 g, 25.7 mmol) and the resulting solution was stirred at 90° C. for 4 h. Solvents were evaporated under reduced pressure and the residue was dissolved in EtOAc (150 mL) and washed with saturated aq. NaHCO₃ (2×150 mL). The organic phase was dried over sodium sulfate, filtered and concentrated under vacuum to give the title compound (10.9 g, yield: 92%) as a pale orange oil. LC-MS (Method A7) m/z [M+H]⁺: 261.0/263.0; rt: 2.24 min; purity: 95%.

### Step 2: Synthesis of ethyl 2-(2-bromo-5-fluorophenyl)propanoate N70_2

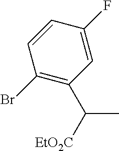

To a stirred solution of ethyl 2-(2-bromo-5-fluoro-phenyl)acetate (Intermediate N70_1, 10.9 g, 39.7 mmol) in dry THF (110 mL) at 0° C. was added a 2M solution of lithium bis(trimethylsilyl)amide in THE (24 mL, 47.8 mmol) and the resulting solution was stirred at 0° C. for 15 min. Iodomethane (8.44 g, 59.5 mmol) was added slowly at 0° C. and the reaction mixture was allowed to warm up to room temperature and stirred for 18 h. The reaction mixture was diluted with EtOAc (140 mL), washed with water (2×120 mL), dried over sodium sulfate, filtered and concentrated under vacuum. The residue was purified by flash chromatography on silica gel (using a gradient of 0 to 100% EtOAc in iso-hexane as eluent) to afford the title compound (11.2 g, yield: 92%) as a pale orange oil. ¹H NMR (400 MHz, CDCl₃) δ 7.52 (dd, J=8.8, 5.4 Hz, 1H), 7.07 (dd, J=9.7, 3.0 Hz, 1H), 6.91-6.80 (m, 1H), 4.26-4.08 (m, 3H), 1.48 (d, J=7.1 Hz, 3H), 1.23 (t, J=7.1 Hz, 3H).

### Step 3: Synthesis of ethyl 2-(5-fluoro-2-(4,4,5,5-tetramethyl-1,3,2-dioxaborolan-2-yl)phenyl)propanoate N70_3

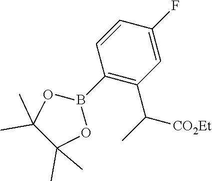 [SMILES: CCOC(=O)C(C)c1cc(F)ccc1B1OC(C)(C)C(C)(C)O1]

To a suspension of ethyl 2-(2-bromo-5-fluoro-phenyl)propanoate (Intermediate N70_2, 11.2 g, 36.6 mmol), bis(pinacolato)diboron (11.2 g, 44.0 mmol) and potassium acetate (14.4 g, 147 mmol) in 1,4-dioxane (110 mL) under nitrogen atmosphere, was added [1,1′-bis(diphenylphosphino)ferrocene]dichloropalladium(II) (1.34 g, 1.83 mmol) and the suspension was heated at 100° C. for 18 h. The reaction mixture was filtered through a pad of Celite® and washed with EtOAc (400 mL). The filtrate was concentrated under vacuum and the residue was purified by flash chromatography on silica gel (using a gradient of 0 to 60% EtOAc in iso-hexane as eluent) to give the title compound (5.40 g, yield: 43%) as colorless oil. ¹H NMR (400 MHz, CDCl₃) δ 7.80 (dd, J=8.4, 6.8 Hz, 1H), 7.01 (dd, J=10.8, 2.5 Hz, 1H), 6.92 (td, J=8.4, 2.5 Hz, 1H), 4.68 (qd, J=7.1, 1.1 Hz, 1H), 4.19-4.04 (m, 2H), 1.46 (d, J=7.1 Hz, 3H), 1.34 (d, J=1.1 Hz, 12H), 1.20 (t, J=7.1 Hz, 3H).

### Step 4: Synthesis of ethyl 2-(2-(4-amino-2-methoxy-6-methylpyridin-3-yl)-5-fluorophenyl)propanoate N70_4

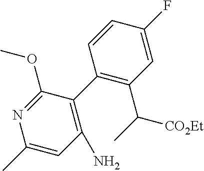 [SMILES: CCOC(=O)C(C)c1cc(F)ccc1-c1c(N)cc(C)nc1OC]

3-Bromo-2-methoxy-6-methylpyridin-4-amine (Intermediate N64_3, 512 mg, 2.36 mmol), ethyl 2-[5-fluoro-2-(4,4,5,5-tetramethyl-1,3,2-dioxaborolan-2-yl)phenyl]propanoate (Intermediate N70_3, 1.00 g, 2.95 mmol) and CsF (764 mg, 5.03 mmol) were dissolved in 1,4-dioxane (25.0 mL) and water (0.5 mL) and the resulting mixture was purged with nitrogen for 5 min. SPhos Pd(crotyl)Cl (CAS 1798781-99-3, 96 mg, 0.157 mmol) was added and the reaction mixture was purged again with nitrogen for 5 min. The reaction mixture was stirred at 90° C. for 18 h. The reaction mixture was cooled to room temperature, diluted in EtOAc (50 mL) and filtered through celite. The filter pad was washed with EtOAc (120 mL) and the filtrate was concentrated under vacuum. The residue was purified by flash chromatography on silica gel (using a gradient of 0 to 100% EtOAc in iso-hexane as eluent) to afford the title compound (517 mg, yield: 53%) as colorless oil. LC-MS (Method B5′) m/z [M+H]⁺: 333.2; rt: 2.14/2.23 min (mixture of atropisomers); purity>99%.

### Step 5: Synthesis of 9-fluoro-1-methoxy-3,7-dimethyl-5,7-dihydropyrido[4,3-d][3]benzazepin-6-one N70_5

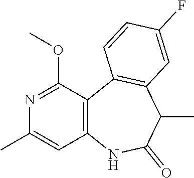

To a stirred solution of ethyl 2-[2-(4-amino-2-methoxy-6-methyl-3-pyridyl)-5-fluoro-phenyl]propanoate (Intermediate N70_4, 517 mg, 1.56 mmol) in EtOH (20.0 mL) was added K₂CO₃ (645 mg, 4.67 mmol) and the reaction mixture was stirred at 90° C. for 18 h. Solvents were evaporated and the crude product was treated with water (60 mL) and extracted with EtOAc (2×30 mL). The combined organic layers were dried over anhydrous Na₂SO₄, filtered and concentrated under vacuum. The residue was triturated with MTBE (5 mL) to afford the title compound (393 mg, yield: 84%) as pale-yellow solid. LC-MS (Method A7) m/z [M+H]⁺: 287.2; rt: 2.09 min; purity: 96%.

### Step 6: Synthesis of ethyl 2-(9-fluoro-1-methoxy-3,7-dimethyl-6-oxo-7H-pyrido[4,3-d][3]benzazepin-5-yl)acetate N70_6

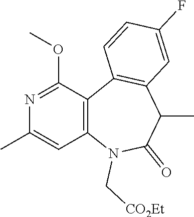

9-Fluoro-1-methoxy-3,7-dimethyl-5,7-dihydropyrido[4,3-d][3]benzazepin-6-one (Intermediate N70_5, 393 mg, 1.32 mmol) was dissolved in dry DMF (20 mL) and potassium carbonate (546 mg, 3.95 mmol) followed by ethyl 2-bromoacetate (440 mg, 2.64 mmol) were added. The reaction mixture was stirred at room temperature for 48 h. The reaction mixture was diluted with water (200 mL) and extracted with EtOAc (2×80 mL). The combined organic extracts were washed with brine (100 mL), dried over anhydrous sodium sulfate, filtered and concentrated under vacuum. The residue was purified by flash chromatography on silica gel (using a gradient of 0 to 100% EtOAc in iso-hexane as eluent) to afford the title compound (365 mg, yield: 72%) as a pale-yellow solid. LC-MS (Method A7) m/z [M+H]⁺: 373.2; rt: 2.47 min; purity: 97%. ¹H NMR (400 MHz, DMSO-d₆) δ 7.74 (dd, J=8.6, 5.9 Hz, 1H), 7.20 (td, J=8.6, 2.7 Hz, 1H), 7.13 (dd, J=10.2, 2.7 Hz, 1H), 6.93 (s, 1H), 4.50-4.36 (m, 2H), 4.00 (qd, J=7.1, 2.7 Hz, 2H), 3.88 (s, 3H), 3.45 (q, J=6.7 Hz, 1H), 2.45 (s, 3H), 1.42 (d, J=6.7 Hz, 3H), 1.05 (t, J=7.1 Hz, 3H).

### Step 7: Synthesis of ethyl 2-(9-fluoro-3,7-dimethyl-1,6-dioxo-2,7-dihydropyrido[4,3-d][3]benzazepin-5-yl)acetate N70_7

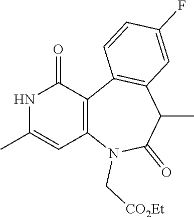

To a stirred solution of ethyl 2-(9-fluoro-1-methoxy-3,7-dimethyl-6-oxo-7H-pyrido[4,3-d][3]benzazepin-5-yl)acetate (Intermediate N70_6, 360 mg, 0.938 mmol) in acetonitrile (20.0 mL) were added KI (467 mg, 2.81 mmol) followed by TMSCl (0.35 mL, 2.80 mmol) and the reaction mixture was stirred at 80° C. for 16 h. The reaction mixture was concentrated under vacuum and the residue was partitioned between 10% MeOH/DCM (50 mL) and water (30 mL). The layers were separated, and the aqueous phase was extracted with 10% MeOH/DCM (2×20 mL). The combined organic extracts were washed with sat. aq. sodium thiosulphate solution (50 mL), filtered through a hydrophobic frit and concentrated under vacuum. The residue was triturated with MTBE (10 mL) to afford the title compound (303 mg, yield: 88%) as a pale-yellow solid. LC-MS (Method A7) m/z [M+H]⁺: 359.3; rt: 1.66 min; purity: 98%.

### Step 8: Synthesis of ethyl 2-(9-fluoro-2,3,7-trimethyl-1,6-dioxo-7H-pyrido[4,3-d][3]benzazepin-5-yl)acetate N70_8

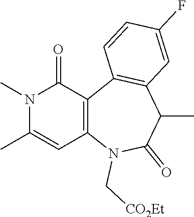

Ethyl 2-(9-fluoro-3,7-dimethyl-1,6-dioxo-2,7-dihydropyrido[4,3-d][3]benzazepin-5-yl)acetate (Intermediate N70_7, 303 mg, 0.829 mmol) was dissolved in dry acetonitrile (15 mL) and cesium carbonate (810 mg, 2.49 mmol) was added. After 10 min, iodomethane (0.15 mL, 2.49 mmol) was slowly added and the reaction mixture was stirred at room temperature for 3 h. The reaction mixture was filtered through Celite® and washed with EtOAc (100 mL) and the filtrate was concentrated under vacuum. The residue was purified by flash chromatography on silica gel (using a gradient of 0 to 5% MeOH in DCM as eluent) to afford the title compound (298 mg, yield: 92%) as a white foam. LC-MS (Method A7) m/z [M+H]⁺: 373.2; rt: 1.81 min; purity: 96%. ¹H NMR (400 MHz, DMSO-d₆) δ 7.85 (dd, J=8.8, 6.1 Hz, 1H), 7.16 (td, J=8.8, 2.7 Hz, 1H), 7.07 (dd, J=10.2, 2.7 Hz, 1H), 6.30 (s, 1H), 4.46-4.27 (m, 2H), 4.04-3.95 (m, 2H), 3.42 (q, J=6.7 Hz, 1H), 3.32 (s, 3H), 2.42 (s, 3H), 1.43 (d, J=6.7 Hz, 3H), 1.04 (t, J=7.1 Hz, 3H).

### Step 9: Synthesis of [2-(9-fluoro-2,3,7-trimethyl-1,6-dioxo-7H-pyrido[4,3-d][3]benzazepin-5-yl)acetyl]oxylithium N70

To a solution of ethyl 2-(9-fluoro-2,3,7-trimethyl-1,6-dioxo-7H-pyrido[4,3-d][3]benzazepin-5-yl)acetate (Intermediate N70_8, 298 mg, 0.760 mmol) in THF (3.0 mL) and MeOH (3.0 mL) was added LiOH·H₂O (39 mg, 0.925 mmol) in water (1.5 mL) and the reaction mixture was stirred at room temperature for 1 h. The reaction mixture was concentrated under vacuum to give the title compound (266 mg, yield: 94%) as a white solid. The product was taken to the next step without further purification. LC-MS (Method A7) m/z [M+2H—Li]⁺: 345.2; rt: 1.45 min; purity>99%.

### Intermediate N70 2A: methyl 2-(2-bromo-5-fluoro-phenyl)propanoate

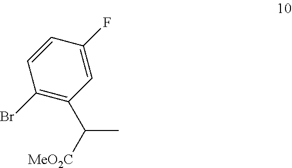

The title compound was prepared according to a procedure similar to the one described for the synthesis of intermediate N70_2 by methylation of methyl 2-(2-bromo-5-fluoro-phenyl)acetate with methyl iodide in the presence of LDA in THF at −78° C.

### Intermediate N71: [2-(4-cyclopropyl-5,10-dimethyl-3,9-dioxo-4,8,12-triazatricyclo[9.4.0.0^{2,7}]pentadeca-1(11),2),5,12,14-pentaen-8-yl)acetyl]oxylithium

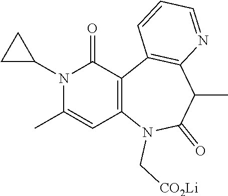

### Step 1: Synthesis of ethyl 2-(4′-amino-2′-methoxy-6′-methyl-[3,3′-bipyridin]-2-yl)propanoate N71_1

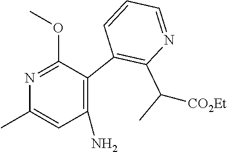 [SMILES: CCOC(=O)C(C)c1ncccc1-c1c(N)cc(C)nc1OC]

To a stirring mixture of 3-bromo-2-methoxy-6-methylpyridin-4-amine (Intermediate N64_3) (1.60 g, 7.37 mmol), ethyl 2-[3-(4,4,5,5-tetramethyl-1,3,2-dioxaborolan-2-yl)-2-pyridyl]propanoate (Intermediate N31_1, 3.29 g, 8.85 mmol) and a 1.5M solution of aq. K₂CO₃ (14.5 mL, 22.0 mmol) in dry 1,4-dioxane (100 mL) was added bis(di-tert-butyl(4-dimethylaminophenyl)phosphine)dichloropalladium(II) (522 mg, 0.737 mmol) and the reaction mixture was stirred at 90° C. for 2 h. The reaction mixture was cooled to room temperature and filtered through a small pad of celite. The filter cake was rinsed with EtOAc (150 mL) and the filtrate was concentrated under vacuum. The residue was purified by flash chromatography on silica gel (using a gradient of 0 to 100% (0.7 M NH₃/MeOH) in DCM as eluent) to afford the title compound (3.24 g, yield: 73%) as a brown oil. LC-MS (Method A7) m/z [M+H]⁺: 316.2; rt: 0.90 min; purity: 53%. ¹H NMR (400 MHz, CDCl₃) δ 8.61 (dd, J=4.8, 1.8 Hz, 1H), 7.50 (dd, J=7.7, 1.8 Hz, 1H), 7.25-7.22 (m, 1H), 6.21 (s, 1H), 4.17-4.02 (m, 2H), 3.86-3.75 (m, 6H), 2.38 (d, J=2.1 Hz, 3H), 1.42 (m, 3H), 1.16 (t, J=7.2 Hz, 3H).

### Step 2: Synthesis of 3-methoxy-5,10-dimethyl-4,8,12-triazatricyclo[9.4.0.0^{2,7}]pentadeca-1(11),2(7),3,5,12,14-hexaen-9-one N71_2

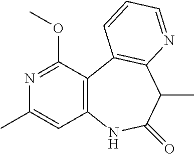

Ethyl 2-[3-(4-amino-2-methoxy-6-methyl-3-pyridyl)-2-pyridyl]propanoate (Intermediate N71_1, 3.24 g, 5.45 mmol) was dissolved in EtOH (70 mL) and potassium carbonate (1.50 g, 10.9 mmol) was added. The reaction mixture was stirred at 90° C. for 16 h. The reaction mixture was concentrated under vacuum and the residue was dissolved in water (30 mL) and extracted with EtOAc (4×50 mL). The combined organic extracts were dried over sodium sulfate, filtered and concentrated under vacuum to afford the title compound (1.99 g, yield: 92%) as a white solid. The product was taken to the next step without further purification. LC-MS (Method A7) m/z [M+H]⁺: 270.2; rt: 1.50 min; purity: 68%. ¹H NMR (400 MHz, DMSO-d₆) δ 10.45 (s, 1H), 8.56 (dd, J=4.7, 1.7 Hz, 1H), 8.11 (dd, J=8.0, 1.7 Hz, 1H), 7.37 (dd, J=7.9, 4.7 Hz, 1H), 6.74-6.64 (m, 1H), 3.89 (s, 3H), 3.46 (d, J=6.6 Hz, 1H), 2.41 (s, 3H), 1.46 (d, J=6.6 Hz, 3H).

### Step 3: Synthesis of ethyl 2-(3-methoxy-5,10-dimethyl-9-oxo-4,8,12-triazatricyclo[9.4.0.0^{2,7}]pentadeca-1(11),2(7),3,5,12,14-hexaen-8-yl)acetate N71_3

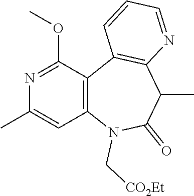

3-Methoxy-5,10-dimethyl-4,8,12-triazatricyclo[9.4.0.0′⁷]pentadeca-1(11),2(7),3,5,12,14-hexaen-9-one (Intermediate N71_2, 1.99 g, 5.02 mmol) was dissolved in dry DMF (50 mL). Potassium carbonate (2.08 g, 15.1 mmol) and ethyl 2-bromoacetate (1.17 mg, 7.03 mmol) were sequentially added and the reaction mixture was stirred at room temperature for 3 h. The reaction mixture was diluted with water (150 mL) and extracted with EtOAc (2×80 mL). The combined organic extracts were washed with brine (100 mL), dried over anhydrous sodium sulfate, filtered and concentrated under vacuum. The residue was purified by flash chromatography on silica gel (using a gradient of 0 to 100% EtOAc in iso-hexane as eluent) to afford the title compound (1.50 g, yield: 65%) as a white solid. LC-MS (Method A7) m/z [M+H]⁺: 356.2; rt: 1.96 min; purity:78%. ¹H NMR (400 MHz, DMSO-d₆) δ 8.57 (dd, J=4.7, 1.7 Hz, 1H), 8.12 (dd, J=7.9, 1.7 Hz, 1H), 7.40 (dd, J=7.9, 4.7 Hz, 1H), 6.98 (s, 1H), 4.56-4.38 (m, 2H), 4.00 (dd, J=7.1, 1.7 Hz, 2H), 3.89 (s, 3H), 3.63 (d, J=6.6 Hz, 1H), 2.46 (s, 3H), 1.46 (d, J=6.6 Hz, 3H), 1.04 (t, J=7.1 Hz, 3H).

### Step 4: Synthesis of ethyl 2-(5,10-dimethyl-3,9-dioxo-4,8,12-triazatricyclo[9.4.0.0^{2,7}]pentadeca-1(11),2(7),5,12,14-pentaen-8-yl)acetate N71_4

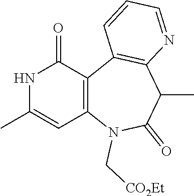

To a stirred solution of ethyl 2-(3-methoxy-5,10-dimethyl-9-oxo-4,8,12-triazatricyclo[9.4.0.0^{2,7}]pentadeca-1(11),2(7),3,5,12,14-hexaen-8-yl)acetate (Intermediate N71_3, 1.50 g, 3.29 mmol) in acetonitrile (30.0 mL) were added KI (1.64 g, 9.88 mmol) followed by TMSCl (1.25 mL, 9.88 mmol) and the reaction mixture was stirred at 80° C. for 16 h. The reaction mixture was concentrated under vacuum and the residue was partitioned between 10% MeOH/DCM (100 mL) and water (80 mL). The layers were separated, and the aqueous phase was extracted with 10% MeOH/DCM (2×50 mL). The combined organic extracts were washed with sat. sodium thiosulphate solution (90 mL), filtered through a hydrophobic frit and concentrated under vacuum. The residue was triturated with MTBE (10 mL) to afford the title compound (754 mg, yield: 66%) as a pale brown solid. LC-MS (Method A7) m/z [M+H]⁺: 342.2; rt: 1.14 min; purity: 99%. ¹H NMR (400 MHz, DMSO-d₆) δ 11.95 (s, 1H), 8.53 (dd, J=4.7, 1.8 Hz, 1H), 8.23 (dd, J=7.9, 1.8 Hz, 1H), 7.36 (dd, J=7.9, 4.7 Hz, 1H), 6.17 (s, 1H), 4.46-4.28 (m, 2H), 4.06-3.95 (m, 2H), 3.58 (q, J=6.6 Hz, 1H), 2.24 (s, 3H), 1.48 (d, J=6.7 Hz, 3H), 1.04 (t, J=7.1 Hz, 3H).

### Step 5: Synthesis of ethyl 2-(4-cyclopropyl-5,10-dimethyl-3,9-dioxo-4,8,12-triazatricyclo[9.4.0.0^{2,7}]pentadeca-1(11),2(7),5,12,14-pentaen-8-yl)acetate N71_5

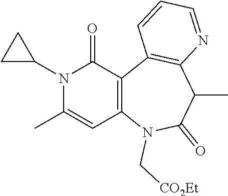 [SMILES: CCOC(=O)CN1C(=O)C(C)c2ncccc2-c2c1cc(C)n(C1CC1)c2=O]

A stirring mixture of ethyl 2-(5,10-dimethyl-3,9-dioxo-4,8,12-triazatricyclo[9.4.0.0²,7]pentadeca-1(11),2(7),5,12,14-pentaen-8-yl)acetate (Intermediate N71_4, 260 mg, 0.754 mmol), cyclopropylboronic acid (389 mg, 4.52 mmol), copper(II)acetate (137 mg, 0.754 mmol), sodium carbonate (240 mg, 2.26 mmol), 2,2′-dipyridyl (118 mg, 0.754 mmol) and 4 A molecular sieves (~0.4 g) in dry DCE (15 mL) was heated at 80° C. for 48 h in open air. The reaction mixture was cooled down to room temperature and filtered through a small pad of celite. The filter cake was rinsed with EtOAc (50 mL) and the filtrate was concentrated under vacuum. The residue was purified by flash chromatography on silica gel (using a gradient of 0 to 100% EtOAc in iso-hexane as eluent) to afford the title compound (104 mg, yield: 35%) as a beige solid. LC-MS (Method A7) m/z [M+H]⁺: 382.2; rt: 1.45 min; purity: 99%. ¹H NMR (400 MHz, DMSO-d₆) δ 8.53 (dd, J=4.7, 1.7 Hz, 1H), 8.19 (dd, J=8.0, 1.7 Hz, 1H), 7.36 (dd, J=7.9, 4.7 Hz, 1H), 6.27 (d, J=0.9 Hz, 1H), 4.45-4.28 (m, 2H), 4.04-3.95 (m, 2H), 3.56 (q, J=6.7 Hz, 1H), 2.98-2.89 (m, 1H), 2.49 (s, 3H, DMSO overlap), 1.47 (d, J=6.7 Hz, 3H), 1.16 (p, J=6.7, 6.3 Hz, 2H), 1.03 (t, J=7.1 Hz, 3H), 0.94-0.86 (m, 1H), 0.82-0.76 (m, 1H).

### Step 6: Synthesis of [2-(4-cyclopropyl-5,10-dimethyl-3,9-dioxo-4,8,12-triazatricyclo[9.4.0.0^{2,7}]pentadeca-1(11),2(7),5,12,14-pentaen-8-yl)acetyl]oxylithium N71

To a solution of ethyl 2-(4-cyclopropyl-5,10-dimethyl-3,9-dioxo-4,8,12-triazatricyclo[9.4.0.0^{2,7}]pentadeca-1(11),2(7),5,12,14-pentaen-8-yl)acetate (Intermediate N71_5, 101 mg, 0.257 mmol) in dry THF (3.0 mL) and MeOH (3.0 mL) was added a solution of LiOH·H₂O (13 mg, 0.313 mmol) in water (1.5 mL) and the reaction mixture was stirred at room temperature for 1 h. The volatiles were removed under vacuum to give the title compound (92 mg, yield: 99%). LC-MS (Method A7) m/z [M+2H—Li]⁺: 354.2; rt: 1.09 min; purity>99%.

### Intermediate N72: [2-(3-fluoro-5-methyl-9,12-dioxo-4,8,13-triazatricyclo[9.4.0.0^{2,7}]pentadeca-1(11),2(7),3,5,14-pentaen-8-yl)acetyl]oxylithium

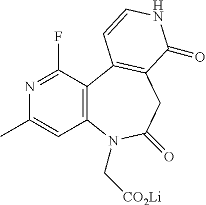

### Step 1: Synthesis of 4-chloro-2-methoxy-3-(2-methoxyvinyl)pyridine N72_1

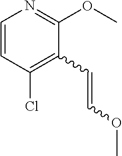

To a mixture of (methoxymethyl)(triphenyl)phosphonium chloride (1.80 g, 5.25 mmol) in toluene (25 mL) was added dropwise at −10° C. a 1M solution of lithium bis(trimethylsilyl)amide in THF (5.2 mL, 5.20 mmol) and the reaction mixture was stirred for 30 min at −10° C. A solution of 4-chloro-2-methoxy-pyridine-3-carbaldehyde (300 mg, 1.75 mmol) in dry toluene (4 mL) was then added dropwise. The reaction mixture was warmed to room temperature and stirred for 2 h. The reaction mixture was treated with a sat. aq. NH₄Cl solution (10 mL) and extracted with EtOAc (3×30 mL). The combined organic extracts were dried over sodium sulfate, filtered and concentrated under vacuum. The residue was purified by flash chromatography on silica gel (using a gradient of 0 to 10% EtOAc in iso-hexane as eluent) to afford the title compound (180 mg, yield: 49%) as an oil that solidified upon standing. Mixture 3:2 of E-Z isomers. LC-MS (Method A7) m/z [M+H]⁺: 200.1/202.1; rt: 2.23 min; purity: 95%. ¹H NMR (400 MHz, DMSO-d₆) [Major isomer]6 7.88 (d, J=5.4 Hz, 1H), 7.60 (d, J=12.7 Hz, 1H), 7.11 (d, J=5.4 Hz, 1H), 5.91 (d, J=12.7 Hz, 1H), 3.94 (s, 3H), 3.70 (s, 3H); ¹H NMR [Minor isomer]; (400 MHz, DMSO-d₆) δ 7.98 (dd, J=5.5, 0.7 Hz, 1H), 7.08 (d, J=5.5 Hz, 1H), 6.37 (d, J=6.6 Hz, 1H), 5.10 (d, J=6.6 Hz, 1H), 3.85 (s, 3H), 3.63 (s, 3H).

### Step 2: Synthesis of 2-(4-chloro-2-methoxy-3-pyridyl)acetaldehyde N72_2

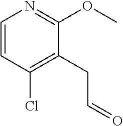

To a solution of 4-chloro-2-methoxy-3-[(E)-2-methoxyvinyl]pyridine (Intermediate N72_1, 100 mg, 0.476 mmol) in acetonitrile (1 mL) was added a 6N aq. solution of HCl (1.4 mL, 8.10 mmol) and the reaction mixture was stirred at room temperature for 18 h. The reaction mixture was neutralized by the addition of solid NaHCO₃(~0.2 g), diluted with water (2 mL) and extracted with EtOAc (3×5 mL). The combined organic extracts were washed with water (5 mL), dried over sodium sulfate, filtered and concentrated under vacuum to afford the title compound (110 mg, yield: 99%) as a colorless oil. LC-MS (Method A7) m/z [M+H]⁺: 186.1/188.0; rt: 1.60 min; purity: 80%.

### Step 3: Synthesis of 2-(4-chloro-2-methoxy-3-pyridyl)acetic acid N72_3

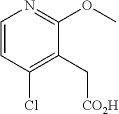

At 0° C., to a solution of 2-(4-chloro-2-methoxy-3-pyridyl)acetaldehyde (Intermediate N72_2, 110 mg, 0.474 mmol) in tert-butanol (1 mL) were sequentially added a 2M aq. solution of NaH₂PO₄ (0.7 mL, 1.42 mmol) and a 2M aq. solution of NaClO₂ (0.8 mL, 1.68 mmol) and the reaction mixture was stirred for 30 min at 0° C., before being diluted with a 1M aq. HCl solution (1 mL). The reaction mixture was warmed to room temperature, diluted with water (5 mL) and extracted with EtOAc (3×10 mL). The combined organic extracts were washed with brine (10 mL), dried over sodium sulfate, filtered and concentrated under vacuum to afford the title compound (122 mg, yield: 66%) as a pale-yellow solid. LC-MS (Method A7) m/z [M+H]⁺: 202.1/204.1; rt: 1.41 min; purity: 82%.

### Step 4: Synthesis of ethyl 2-(4-chloro-2-methoxy-3-pyridyl)acetate N72_4

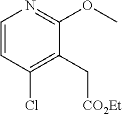

To a solution of 2-(4-chloro-2-methoxy-3-pyridyl)acetic acid (Intermediate N72_3, 117 mg, 0.476 mmol) in EtOH (1 mL) was added concentrated H₂SO₄ (13 μL, 0.238 mmol) and the reaction mixture was stirred at 70° C. for 18 h. The reaction mixture was concentrated under vacuum and the residue was neutralized by the addition of sat. aq. NaHCO₃ solution (3 mL) and extracted with EtOAc (2×3 mL). The combined organic extracts were dried over sodium sulfate, filtered and concentrated under vacuum. The residue was purified by flash chromatography on silica gel (using a gradient of 0 to 50% EtOAc in iso-hexane as eluent) to afford the title compound (31 mg, yield: 27%) as a colorless oil. LC-MS (Method A7) m/z [M+H]⁺: 230.2/232.1; rt: 2.02 min; purity: 98%. ¹H NMR (400 MHz, DMSO-d₆) δ 8.08 (d, J=5.5 Hz, 1H), 7.16 (d, J=5.5 Hz, 1H), 4.09 (q, J=7.1 Hz, 2H), 3.88 (s, 3H), 3.74 (s, 2H), 1.17 (t, J=7.1 Hz, 3H).

### Step 5: Synthesis of ethyl 2-[4-(4-amino-2-fluoro-6-methyl-3-pyridyl)-2-methoxy-3-pyridyl]acetate N72_5

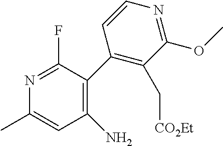

A mixture of ethyl 2-(4-chloro-2-methoxy-3-pyridyl)acetate (Intermediate N72_4, 29 mg, 0.123 mmol), 2-fluoro-6-methyl-3-(4,4,5,5-tetramethyl-1,3,2-dioxaborolan-2-yl)pyridin-4-amine (Intermediate N16_1, 47 mg, 0.160 mmol), SPhos Pd(crotyl)Cl (CAS 1798781-99-3, 7 mg, 0.012 mmol) and CsF (56 mg, 0.368 mmol) in a solvent mixture of 1,4-dioxane (5 mL) and water (1 mL) was heated at 90° C. for 2 h. The reaction mixture was cooled to room temperature, filtered through a small pad of Celite® and the filtrate was concentrated under vacuum to afford the title compound (130 mg, quantitative yield). The product was taken to the next step without purification. LC-MS (Method A7) m/z [M+H]⁺: 320.2; rt: 1.63 min; purity: 83%.

### Step 6: Synthesis of 3-fluoro-12-methoxy-5-methyl-4,8,13-triazatricyclo[9.4.0.0^{2,7}]pentadeca-1(11),2(7),3,5,12,14-hexaen-9-one N72_6

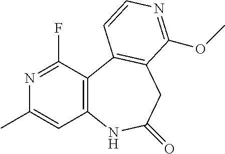

At 0° C., to a solution of ethyl 2-[4-(4-amino-2-fluoro-6-methyl-3-pyridyl)-2-methoxy-3-pyridyl]acetate (Intermediate N72_5, 130 mg, 0.123 mmol) in dry THF (3 mL) was added dropwise a 1M solution of lithium bis(trimethylsilyl)amide in THF (0.4 mL, 0.370 mmol) and the reaction mixture was stirred for 30 min at 0° C. The reaction mixture was treated with sat. NH₄Cl solution (5 mL) and extracted with EtOAc (3×5 mL). The combined organic extracts were washed with brine (5 mL), dried over sodium sulfate, filtered and concentrated under vacuum. The residue was purified by flash chromatography on silica gel (using a gradient of 0 to 100% EtOAc in iso-hexane as eluent) to afford the title compound (41 mg, yield: 99%). LC-MS (Method A7) m/z: [M+H]⁺: 274.2; rt: 1.54 min; purity: 87%.

### Step 7: Synthesis of ethyl 2-(3-fluoro-12-methoxy-5-methyl-9-oxo-4,8,13-triazatricyclo[9.4.0.0^{2,7}]pentadeca-1(11),2(7),3,5,12,14-hexaen-8-yl)acetate N72_7

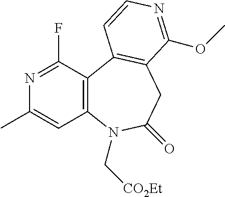

To a solution of 3-fluoro-12-methoxy-5-methyl-4,8,13-triazatricyclo[9.4.0.0²,7]pentadeca-1(11),2(7),3,5,12,14-hexaen-9-one (Intermediate N72_6, 41 mg, 0.123 mmol) in dry DMF (2 mL) were added potassium carbonate (34 mg, 0.246 mmol) and KI (2 mg, 0.012 mmol). After 10 min, ethyl 2-bromoacetate (25 mg, 0.148 mmol) was slowly added and the reaction mixture was stirred at room temperature for 16 h. The reaction mixture was treated with water (10 mL) and extracted with EtOAc (3×5 mL). The combined organic extracts were washed with brine (5 mL), dried over sodium sulfate, filtered and concentrated under vacuum. The residue was purified by flash chromatography on silica gel (using a gradient of 0 to 10% (0.7 M NH₃/MeOH) in DCM as eluent) to afford the title compound (42 mg, yield: 87%) as a colorless oil. LC-MS (Method A7) m/z [M+H]⁺: 360.2; rt: 1.90 min; purity: 92%.

### Step 8: Synthesis of ethyl 2-(3-fluoro-5-methyl-9,12-dioxo-4,8,13-triazatricyclo[9.4.0.0′⁷]pentadeca-1(11),2(7),3,5,14-pentaen-8-yl)acetate N72_8

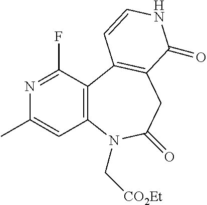 [SMILES: CCOC(=O)CN1C(=O)Cc2c(cc[nH]c2=O)-c2c1cc(C)nc2F]

A suspension of ethyl 2-(3-fluoro-12-methoxy-5-methyl-9-oxo-4,8,13-triazatricyclo[9.4.0.0^{2,7}]pentadeca-1(11),2(7),3,5,12,14-hexaen-8-yl)acetate (Intermediate N72_7, 40 mg, 0.102 mmol), KI (85 mg, 0.512 mmol) and TMSCl (0.065 mL, 0.512 mmol) in dry MeCN (5.0 mL) was stirred at 80° C. for 2 h. The reaction mixture was concentrated under vacuum and the residue was purified by flash chromatography on silica gel (using a gradient of 0 to 10% (0.7 M (NH₃/MeOH) in DCM as eluent) to afford the title compound (25 mg, yield: 68%) as a white solid. LC-MS (Method A7) m/z [M+H]⁺: 346.1; rt: 1.37 min; purity: 97%.

### Step 9: Synthesis of [2-(3-fluoro-5-methyl-9,12-dioxo-4,8,13-triazatricyclo[9.4.0.0²⁷]pentadeca-1(11),2(7),3,5,14-pentaen-8-yl)acetyl]oxylithium N72

To a solution of ethyl 2-(3-fluoro-5-methyl-9,12-dioxo-4,8,13-triazatricyclo[9.4.0.0²,7]pentadeca-1(11),2(7),3,5,14-pentaen-8-yl)acetate (Intermediate N72_8, 25 mg, 0.070 mmol) in THF (2.0 mL), MeOH (2.0 mL) and water (1 mL) was added LiOH·H₂O (4 mg, 0.105 mmol) and the reaction mixture was stirred at room temperature for 2 h. The reaction mixture was concentrated under vacuum and then co-evaporated with toluene (5 mL) to afford the title compound as a white solid (31 mg, yield: 99%). LC-MS (Method A7) m/z [M+2H—Li]⁺: 318.1; rt: 1.10 min; purity: 72%.

### Intermediate N73: [2-(9-chloro-1,3,7-trimethyl-2,6-dioxo-7H-pyrido[3,2-d][3]benzazepin-5-yl)acetyl]oxylithium

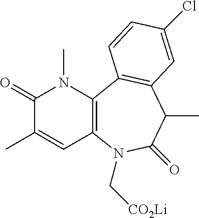

### Step 1: Synthesis of methyl 2-(2-bromo-5-chloro-phenyl)propanoate N73_1

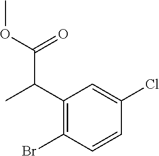

To a solution of methyl 2-(2-bromo-5-chloro-phenyl)acetate (5.0 g, 19.0 mmol) in dry THF (30.0 mL) cooled in an ice/salt bath to 0° C. was added over 20 min a 1M solution of lithium bis(trimethylsilyl)amide in THF (23 mL, 23.0 mmol) and the reaction mixture was stirred for 10 min. Always at 0° C., iodomethane (4.04 g, 28.5 mmol) was added dropwise and the reaction mixture was stirred at 0° C. for 1 h and then at room temperature for 48 h. The reaction mixture was treated with sat. aq. NH₄Cl (60 mL) and extracted with EtOAc (3×50 mL). The organic layers were combined, washed with brine (50 mL), dried over sodium sulfate, filtered and concentrated under vacuum. The residue was purified by flash chromatography on silica gel (using a gradient of 0 to 30% EtOAc in iso-hexane as eluent) to afford the title compound as an opaque oil (4.69 g, yield: 78%). LC-MS (Method A7) m/z [M+H]⁺: 277.0/279.0/281.0; rt: 2.07 min; purity: 88%. ¹H NMR (400 MHz, DMSO-d₆) δ 7.66 (d, J=8.6 Hz, 1H), 7.41 (d, J=2.6 Hz, 1H), 7.31 (dd, J=8.5, 2.6 Hz, 1H), 4.12 (q, J=7.2 Hz, 1H), 3.62 (s, 3H), 1.43 (d, J=7.2 Hz, 3H).

### Step 2: Synthesis of methyl 2-[5-chloro-2-(4,4,5,5-tetramethyl-1,3,2-dioxaborolan-2-yl)phenyl]propanoate N73_2

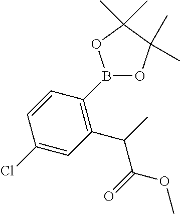 [SMILES: COC(=O)C(C)c1cc(Cl)ccc1B1OC(C)(C)C(C)(C)O1]

To a solution of methyl 2-(2-bromo-5-chloro-phenyl)propanoate (Intermediate N73_1, 4.69 g, 14.9 mmol) in dry 1,4-dioxane (60 mL), were added bis(pinacolato)diboron (CAS 73183-34-3, 4.17 g, 16.4 mmol), potassium acetate (5.12 g, 52.2 mmol) and [1,1′-bis(diphenylphosphino)ferrocene]dichloropalladium(II) (655 mg, 0.895 mmol). The reaction mixture was flushed with nitrogen for 10 min and then heated at 90° C. for 16 h. The reaction mixture was cooled to room temperature, diluted with EtOAc (50 mL), filtered through Celite® and the filtrate was concentrated under vacuum. The residue was purified by flash chromatography on silica gel (using a gradient of 0 to 30% EtOAc in iso-hexane as eluent) to afford the title compound as a clear light-yellow oil (1.33 g, yield: 25%). LC-MS (Method A7) m/z [M+H]⁺: 325.2/327.7; rt: 2.38 min; purity: 92%. ¹H NMR (400 MHz, DMSO-d₆) δ 7.67 (d, J=8.0 Hz, 1H), 7.34 (dd, J=8.0, 2.1 Hz, 1H), 7.28 (d, J=2.1 Hz, 1H), 4.51 (q, J=7.2 Hz, 1H), 3.58 (s, 3H), 1.40 (d, J=7.2 Hz, 3H), 1.29 (s, 12H).

### Step 3: Synthesis of 9-chloro-2-methoxy-3,7-dimethyl-5,7-dihydropyrido[3,2-d][3]benzazepin-6-one N73_3

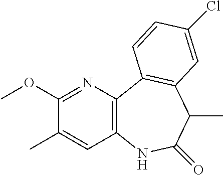

A stirring mixture of 2-bromo-6-methoxy-5-methylpyridin-3-amine (Intermediate N68_1, 130 mg, 0.587 mmol), methyl 2-[5-chloro-2-(4,4,5,5-tetramethyl-1,3,2-dioxaborolan-2-yl)phenyl]propanoate (Intermediate N73_2, 312 mg, 0.880 mmol), bis(di-tert-butyl(4-dimethylaminophenyl)phosphine)dichloropalladium(II) (21 mg, 0.029 mmol) and CsF (267 mg, 1.76 mmol) in 1,4-dioxane (10 mL) and water (0.7 mL) was degassed with nitrogen for 5 min and then heated at 90° C. for 18 h. The reaction mixture was filtered through a small pad of Celite® and the filter cake was rinsed with EtOAc (80 mL). The filtrate was concentrated under vacuum and the residue was purified by flash chromatography on silica gel (using a gradient of 0 to 50% EtOAc in iso-hexane as eluent) to afford the title compound as a gummy light brown solid (35 mg, yield: 19%). LC-MS (Method A7) m/z [M+H]⁺: 303.1/305.1; rt: 2.40 min; purity: 98%. ¹H NMR (400 MHz, DMSO-d₆) δ 10.05 (s, 1H), 7.94 (d, J=8.3 Hz, 1H), 7.52 (dd, J=8.3, 2.1 Hz, 1H), 7.35 (s, 2H), 3.97 (s, 3H), 3.28 (s, 1H), 2.21 (s, 3H), 1.46 (d, J=6.8 Hz, 3H).

### Step 4: Synthesis of ethyl 2-(9-chloro-2-methoxy-3,7-dimethyl-6-oxo-7H-pyrido[3,2-d][3]benzazepin-5-yl)acetate N73_4

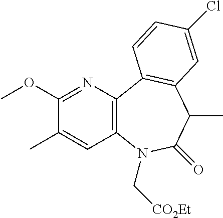

To a solution of 9-chloro-2-methoxy-3,7-dimethyl-5,7-dihydropyrido[3,2-d][3]benzazepin-6-one (Intermediate N73 3. 34 mg, 0.110 mmol) in dry DMF (1.0 mL) were added potassium carbonate (30 mg, 0.220 mmol) and KI (2.0 mg, 0.011 mmol). After 10 min, ethyl bromoacetate (22 mg, 0.132 mmol) was slowly added and the reaction mixture was stirred at room temperature for 16 h. The reaction mixture was treated with water (10 mL) and the resulting precipitate was collected by filtration, washed with water (10 mL) and dried under vacuum at 40° C. to afford the title compound as a brown solid (28 mg, yield: 64%). LC-MS (Method A7) m/z [M+H]⁺: 389.2/391.1; rt: 2.69 min; purity: 98%. ¹H NMR (400 MHz, CDCl₃) δ 7.94 (d, J=8.3 Hz, 1H), 7.41 (dd, J=8.4, 2.1 Hz, 1H), 7.38 (d, J=1.0 Hz, 1H), 7.36-7.33 (m, 1H), 4.41 (d, J=17.2 Hz, 1H), 4.20-4.09 (m, 3H), 4.04 (s, 3H), 3.50 (q, J=6.9 Hz, 1H), 2.26 (d, J=0.9 Hz, 3H), 1.60 (d, J=6.9 Hz, 3H), 1.16 (t, J=7.1 Hz, 3H).

### Step 5: Synthesis of ethyl 2-(9-chloro-3,7-dimethyl-2,6-dioxo-1,7-dihydropyrido[3,2-d][3]benzazepin-5-yl)acetate N73_5

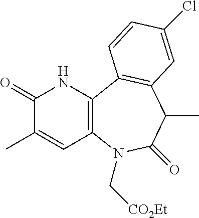

A suspension of ethyl 2-(9-chloro-2-methoxy-3,7-dimethyl-6-oxo-7H-pyrido[3,2-d][3]benzazepin-5-yl)acetate (Intermediate N73_4, 28 mg, 0.071 mmol), KI (35 mg, 0.212 mmol) and TMSCl (0.030 mL, 0.215 mmol) in dry acetonitrile (5.0 mL) was heated at 80° C. for 16 h. The reaction mixture was concentrated under vacuum and the residue was partitioned between EtOAc (20 mL) and water (20 mL). The layers were separated, and the aqueous phase was extracted with EtOAc (3×20 mL). The combined organic extracts were washed with sat. aq. sodium thiosulphate solution (30 mL), dried over magnesium sulphate, filtered and concentrated under vacuum to afford the title compound as a light brown solid (25 mg, yield: 88%). LC-MS (Method A7) m/z [M+H]⁺: 375.1/377.2; rt: 1.71 min; purity: 97%. ¹H NMR (400 MHz, DMSO-d₆) δ 11.87 (s, 1H), 7.69 (s, 1H), 7.51 (dd, J=8.4, 2.1 Hz, 2H), 7.34 (d, J=2.1 Hz, 1H), 4.42 (d, J=17.4 Hz, 1H), 4.24 (d, J=17.4 Hz, 1H), 3.97-3.88 (m, 2H), 3.56 (d, J=6.9 Hz, 1H), 2.08 (s, 3H), 1.45 (d, J=6.9 Hz, 3H), 0.97 (t, J=7.1 Hz, 3H).

### Step 6: Synthesis of ethyl 2-(9-chloro-1,3,7-trimethyl-2,6-dioxo-7H-pyrido[3,2-d][3]benzazepin-5-yl)acetate N73_6

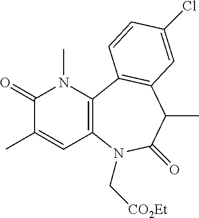

To a solution of ethyl 2-(9-chloro-3,7-dimethyl-2,6-dioxo-1,7-dihydropyrido[3,2-d][3]benzazepin-5-yl)acetate (Intermediate N73_5, 25 mg, 0.063 mmol) in dry acetonitrile (1.0 mL), cesium carbonate (61 mg, 0.188 mmol) was added. After 10 min, a 1M solution of iodomethane in DCM (0.125 mL, 0.125 mmol) was slowly added and the reaction mixture was stirred at room temperature for 16 h. The reaction mixture was concentrated under vacuum and then taken up in DMSO (1.5 mL) and purified by reverse phase chromatography on C18 silica gel (using a gradient of 10 to 50% water in acetonitrile as eluent) to afford the title compound as pale orange powder (8 mg, yield: 34%). LC-MS (Method A7) m/z [M+H]⁺: 389.2/391.2; rt: 1.90 min; purity: 99%. ¹H NMR (400 MHz, DMSO-d₆) δ 7.73 (d, J=8.4 Hz, 1H), 7.52 (d, J=1.3 Hz, 1H), 7.48 (dd, J=8.4, 2.1 Hz, 1H), 7.38 (d, J=2.1 Hz, 1H), 4.41 (d, J=17.3 Hz, 1H), 4.19 (d, J=17.3 Hz, 1H), 3.96-3.85 (m, 2H), 3.74 (q, J=6.8 Hz, 1H), 3.33 (s, 3H), 2.10 (d, J=1.1 Hz, 3H), 1.45 (d, J=6.8 Hz, 3H), 0.99 (t, J=7.1 Hz, 3H).

### Step 7: Synthesis of [2-(9-chloro-1,3,7-trimethyl-2,6-dioxo-7H-pyrido[3,2-d][3]benzazepin-5-yl)acetyl]oxylithium N73

To a solution of ethyl 2-(9-chloro-1,3,7-trimethyl-2,6-dioxo-7H-pyrido[3,2-d][3]benzazepin-5-yl)acetate (Intermediate N73_6, 9 mg, 0.021 mmol) in THF (1.0 mL), MeOH (1.0 mL) and water (0.5 mL) was added LiOH·H₂O (2.0 mg, 0.043 mmol) and the reaction mixture was stirred at room temperature for 16 h. The reaction mixture was concentrated under vacuum and then co-evaporated with acetonitrile (3×10 mL). The residue was triturated with MTBE (5 mL), collected, washed with MTBE (5 mL) and dried under vacuum to afford the title compound as a pale-yellow powder (8 mg, yield: 98%). LC-MS (Method A7) m/z [M-Li+2H]⁺: 361.1/363.2; rt: 1.58 min; purity: 99%.

### Intermediate N74: 4-(2,2-difluorocyclopropyl)aniline

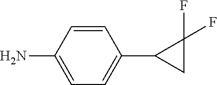 [SMILES: Nc1ccc(C2CC2(F)F)cc1]

### Step 1: Synthesis of 1-(2,2-difluorocyclopropyl)-4-nitrobenzene N74_1

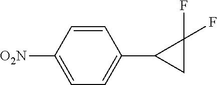 [SMILES: O=[N+]([O-])c1ccc(C2CC2(F)F)cc1]

To a solution of 1-nitro-4-vinyl-benzene (2.00 g, 13.4 mmol) in dry THF (40 mL) was added trimethyl(trifluoromethyl)silane (4.77 g, 33.5 mmol) and NaI (0.40 g, 2.68 mmol) at 0° C. and the reaction mixture was stirred at room temperature for 16 h. The reaction mixture was treated with saturated aq. NaHCO₃ (120 mL) and extracted with DCM (3×200 mL). The organic layer was separated, dried over anhydrous Na₂SO₄ and concentrated under vacuum. The residue was purified by column chromatography on silica gel (using 10% EtOAc in hexanes as eluent) to give the title compound (1.20 g, yield: 45%) as a colorless oil. ¹H NMR (400 MHz, DMSO-d₆) δ 2.01-2.24 (m, 2H) 3.13-3.27 (m, 1H) 7.57 (d, J=8.3 Hz, 2H) 8.20 (d, J=8.3 Hz, 2H).

### Step 2: Synthesis of 4-(2,2-difluorocyclopropyl)aniline N74

To a solution of 1-(2,2-difluorocyclopropyl)-4-nitrobenzene (Intermediate N74_1, 0.50 g, 2.51 mmol) in acetic acid (15 mL) was added iron dust (0.28 g, 5.02 mmol) and the reaction mixture was stirred at room temperature for 2 h. The reaction mixture was filtered off and the filtrate was concentrated under vacuum. The residue was diluted with EtOAc (30 mL) and treated with water (50 mL). The organic layer was separated, dried over anhydrous Na₂SO₄ and concentrated under vacuum. The crude product was purified by column chromatography on silica gel (using 20% EtOAc in hexanes as eluent) to afford the title compound (0.32 g, yield: 74%) as an off-white solid. ¹H NMR (400 MHz, DMSO-d₆) δ 1.68 (dt, J=8.4, 3.8 Hz, 1H) 1.74-1.91 (m, 1H) 2.68-2.84 (m, 1H) 5.02 (bs, 2H) 6.52 (d, J=7.8 Hz, 2H) 6.90 (d, J=7.8 Hz, 2H).

### Intermediate N75: 3-fluoro-5-methyl-10-(2-trimethylsilylethoxymethyl)-4,8,12-triazatricyclo[9.4.0.0^{2,7}]pentadeca-1(11),2(7),3,5,12,14-hexaen-9-one

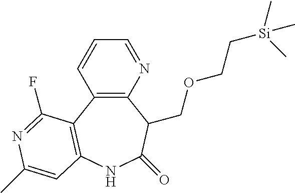

### Step 1: Synthesis of ethyl 2-(3-bromo-2-pyridyl)-3-(2-trimethylsilylethoxy)propanoate N75_1

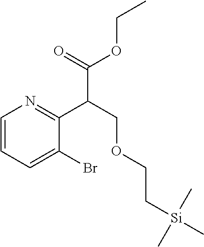 [SMILES: CCOC(=O)C(COCC[Si](C)(C)C)c1ncccc1Br]

At −60° C., to a solution of ethyl 2-(3-bromo-2-pyridyl)acetate (Intermediate N3_1, 2.00 g, 8.19 mmol) in dry THF (60 mL) was added a 2 M solution of lithium diisopropylamide in THF (4.5 mL, 9.01 mmol) over 20 min maintaining the temperature at −60° C. Upon completion of addition, the reaction mixture was stirred at this temperature for a further 20 min before dropwise addition of 2-(trimethylsilyl)ethoxymethyl chloride (1.6 mL, 9.01 mmol). Upon completion of addition, the reaction mixture was allowed to warm to room temperature over 1 hour. The reaction mixture was poured onto water (50 mL) and extracted with EtOAc (2×30 mL). The organic extracts were combined, dried over sodium sulfate, filtered and concentrated under vacuum. The crude product was purified by flash chromatography on silica gel (using a gradient of 0 to 10% MTBE in iso-hexane as eluent) to afford the title compound (2.71 g, yield: 87%) as a colourless oil. ¹H NMR (400 MHz, CDCl₃) δ 8.51 (dd, J=4.6, 1.6 Hz, 1H), 7.86 (dd, J=8.1, 1.6 Hz, 1H), 7.07 (dd, J=8.0, 4.6 Hz, 1H), 4.63 (t, J=7.1 Hz, 1H), 4.17 (q, J=7.1 Hz, 2H), 4.13-4.07 (m, 1H), 3.94 (dd, J=9.5, 6.8 Hz, 1H), 3.64-3.48 (m, 2H), 1.20 (t, J=7.1 Hz, 3H), 0.89 (dd, J=8.6, 7.7 Hz, 2H), −0.06 (s, 9H).

### Step 2: Synthesis of ethyl 2-[3-(4-amino-2-fluoro-6-methyl-3-pyridyl)-2-pyridyl]-3-(2-trimethylsilylethoxy)propanoate N75_2

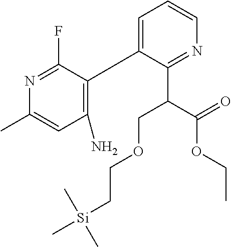 [SMILES: CCOC(=O)C(COCC[Si](C)(C)C)c1ncccc1-c1c(N)cc(C)nc1F]

To a stirred solution of ethyl 2-(3-bromo-2-pyridyl)-3-(2-trimethylsilylethoxy)propanoate (N75_1, 400 mg, 0.705 mmol) in 1,4-dioxane (40.0 mL) and water (4.0 mL) was added 2-fluoro-6-methyl-3-(4,4,5,5-tetramethyl-1,3,2-dioxaborolan-2-yl)pyridin-4-amine (Intermediate N16_1, 178 mg, 0.705 mmol), SPhos Pd(crotyl)Cl (CAS: 1798781-99-3, 43 mg, 0.071 mmol) and cesium fluoride (321 mg, 2.12 mmol) and reaction mixture was stirred at 90° C. for 4 hours. The reaction mixture was cooled to room temperature, poured onto water (150 mL) and extracted with EtOAc (100 mL). The organic extract was separated, dried over sodium sulfate, filtered and concentrated under vacuum. The product was purified by flash chromatography on silica gel (using a gradient of 0 to 50% MTBE in iso-hexane as eluent) to afford the title compound (281 mg, yield: 63%) as a pale yellow oil. LC-MS (Method A7) m/z: [M+H]⁺: 420; rt: 2.33 min; purity: 80%. ¹H NMR (400 MHz, DMSO-d₆) δ 8.55 (dd, J=4.8, 1.9 Hz, 1H), 7.57 (dd, J=7.7, 1.8 Hz, 1H), 7.38 (dd, J=7.6, 4.8 Hz, 1H), 6.45 (s, 1H), 5.92 (s, 2H), 4.08-3.81 (m, 5H), 3.42-3.30 (m, 3H), 2.24 (s, 3H), 1.01 (t, J=7.1 Hz, 2H), 0.78-0.68 (m, 2H), −0.07 (s, 9H).

### Step 3: Synthesis of 3-fluoro-5-methyl-10-(2-trimethylsilylethoxymethyl)-4,8,12-triazatricyclo[9.4.0.0^{2,7}]pentadeca-1(11),2(7),3,5,12,14-hexaen-9-one N75

A mixture of ethyl 2-[3-(4-amino-2-fluoro-6-methyl-3-pyridyl)-2-pyridyl]-3-(2-trimethylsilylethoxy)propanoate (N75 2, 220 mg, 0.419 mmol) and potassium carbonate (232 mg, 1.68 mmol) in ethanol (20.0 mL) was heated at 80° C. for 3 hours. The reaction mixture was concentrated to dryness and to the residue was added water (10 mL) and the pH was adjusted to 5 by the addition of acetic acid. The resulting precipitate was collected by filtration and dried by suction. The solid was purified by flash chromatography on silica gel (using a gradient of 0 to 30% EtOAc in iso-hexane as eluent) to afford the title compound (85 mg, yield: 54%) as a white solid. LC-MS (Method A7) m/z: [M+H]⁺: 374; rt: 2.35 min; purity: 98%. ¹H NMR (400 MHz, DMSO-d₆) δ 10.90 (s, 1H), 8.62 (dd, J=4.8, 1.7 Hz, 1H), 8.10 (ddd, J=8.0, 4.5, 1.7 Hz, 1H), 7.46 (dd, J=8.0, 4.7 Hz, 1H), 7.00 (s, 1H), 4.28-4.14 (m, 2H), 3.60-3.50 (m, 3H), 2.46 (s, 3H), 0.82 (td, J=7.6, 1.2 Hz, 2H), −0.05 (s, 9H).

### Intermediate N76: 3-fluoro-10-methoxy-5-methyl-4,8,12-triazatricyclo[9.4.0.0^{2,7}]pentadeca-1(11),2(7),3,5,12,14-hexaen-9-one

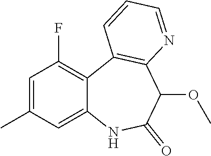

### Step 1: Synthesis of ethyl 2-bromo-2-(3-bromo-2-pyridyl)acetate #159_1

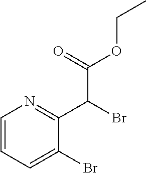

A mixture of ethyl 2-(3-bromo-2-pyridyl)acetate (Intermediate N3_1, 1.00 g, 4.10 mmol), 2,2′-azobis(2-methylpropionitrile) (67 mg, 0.410 mmol) and N-bromosuccinimide (729 mg, 4.10 mmol) in chloroform (30.0 mL) was heated under reflux for 24 hours. The reaction mixture was allowed to cool to room temperature, concentrated to dryness and the residue was purified by flash chromatography on silica gel (using a gradient of 0 to 40% MTBE in iso-hexane as eluent) to afford the title compound (661 mg, yield: 48%) as a colourless oil. LC-MS (Method A7) m/z: [M+H]⁺: 322/324/326; rt: 1.94 min; purity: 95%. ¹H NMR (400 MHz, CDCl₃) δ 8.55 (dd, J=4.6, 1.5 Hz, 1H), 7.90 (dd, J=8.1, 1.5 Hz, 1H), 7.15 (dd, J=8.1, 4.6 Hz, 1H), 5.99 (s, 1H), 4.30 (q, J=7.1 Hz, 2H), 1.27 (t, J=7.1 Hz, 3H).

### Step 2: Synthesis of methyl 2-(3-bromo-2-pyridyl)-2-methoxy-acetate N76_2

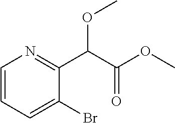

To a solution of sodium methoxide prepared from sodium metal (402 mg, 17.5 mmol) and methanol (100 mL) was added methyl 2-bromo-2-(3-bromo-2-pyridyl)acetate (N76_1, 6.00 g, 17.5 mmol) and the reaction mixture was stirred at room temperature for 24 hours, before being neutralized by the addition of glacial acetic acid (~800 μL). The reaction mixture was concentrated to dryness and the residue was purified by flash chromatography on silica gel (using a gradient of 0 to 70% MTBE in iso-hexane as eluent) to afford the title compound (3.71 g, yield: 80%) as a pale-yellow oil. ¹H NMR (400 MHz, CDCl₃) δ 8.55 (dd, J=4.6, 1.5 Hz, 1H), 7.91 (dd, J=8.1, 1.5 Hz, 1H), 7.16 (dd, J=8.1, 4.6 Hz, 1H), 5.37 (s, 1H), 3.80 (s, 3H), 3.54 (s, 3H).

### Step 3: Synthesis of methyl 2-[3-(4-amino-2-fluoro-6-methyl-3-pyridyl)-2-pyridyl]-2-methoxy-acetate N76_3

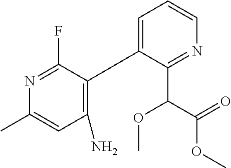

A mixture of methyl 2-(3-bromo-2-pyridyl)-2-methoxy-acetate (N76_2, 500 mg, 1.54 mmol), 2-fluoro-6-methyl-3-(4,4,5,5-tetramethyl-1,3,2-dioxaborolan-2-yl)pyridin-4-amine (Intermediate N16_1, 606 mg, 2.40 mmol) and cesium fluoride (701 mg, 4.61 mmol) in a solvent mixture of 1,4-dioxane (60.0 mL) and water (6.0 mL) was degassed with nitrogen for 15 min. SPhos Pd(crotyl)Cl (CAS: 1798781-99-3, 93 mg, 0.154 mmol) was added and the reaction mixture was heated at 90° C. for 4 hours. The reaction mixture was concentrated to dryness and the residue was taken into EtOAc (30 mL) and washed with water (20 mL). The organic layer was separated, dried over sodium sulfate, filtered and concentrated under vacuum. The crude product was purified by flash chromatography on silica gel (using a gradient of 0 to 100% EtOAc in iso-hexane as eluent) to give the title compound (410 mg, yield: 71%) as yellow oil. LC-MS (Method A7) m/z: [M+H]⁺: 306; rt: 1.08 min; purity: 98%.

### Step 4: Synthesis of 3-fluoro-10-methoxy-5-methyl-4,8,12-triazatricyclo[9.4.0.02 ⁷]pentadeca-1(11),2(7),3,5,12,14-hexaen-9-one N76

A mixture of methyl 2-[3-(4-amino-2-fluoro-6-methyl-3-pyridyl)-2-pyridyl]-2-methoxy-acetate (N76 3, 400 mg, 1.05 mmol) and potassium carbonate (579 mg, 4.19 mmol) in ethanol (50.0 mL) was heated at 80° C. for 3 hours. The reaction mixture was concentrated to dryness and to the residue was added water (20 mL). The aqueous solution was adjusted to pH=−4-5 by the addition of acetic acid. The aqueous mixture was concentrated to dryness and the residue was extracted with EtOAc (200 mL). The extract was filtered to remove inorganics and the filtrate was concentrated to dryness to afford the title compound (279 mg, yield: 96%) as a beige powder. LC-MS (Method B5′) m/z: [M+H]⁺: 274; rt: 1.15 min; purity: 98%.

### Intermediate N77: 7,12-dimethyl-5,6,9,13-tetrazatricyclo[8.4.0.0^{2,7}]tetradeca-1(10),2,4,11,13-pentaen-8-one

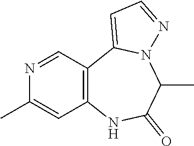

### Step 1: Preparation of 2-methyl-5-(2-tetrahydropyran-2-ylpyrazol-3-yl)pyridin-4-amine N77_1

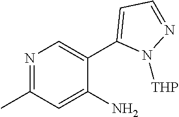

In a pressure-resistant reactor, to a mixture of 4-amino-5-bromo-2-methylpyridine (6.0 g, 31.4 mmol) and 1-(2-tetrahydropyranyl)-1H-pyrazole-5-boronic acid pinacol ester (CAS 903550-26-5, 13.2 g, 47.1 mmol) in dry toluene (300 mL) was added K₃PO₄ (20.6 g, 94.2 mmol) and the reaction mixture was purged with nitrogen for 5 min before the addition of Pd₂dba₃ (1.48 g, 1.57 mmol) and SPhos (1.32 g, 3.14 mmol). The reaction mixture was then stirred at 100° C. for 4 h. After cooling down to RT, the reaction mixture was diluted with EtOAc, filtered through a pad of Celite® and the filtrate was concentrated to dryness. The crude solid was purified by column chromatography on silica gel (using a gradient of 0% to 10% MeOH in DCM as eluent) to afford the title compound as a beige solid (7.31 g, yield: 89%). LC-MS (Method A1) m/z [M+H]⁺: 259.1; rt: 0.69 min; purity: 84%. LC-MS (Method B1) m/z [M+H]⁺: 259.1; rt: 0.86 min; purity: 98%. ¹H NMR (400 MHz, DMSO-d₆) δ 7.86 (s, 1H), 7.62 (d, J=1.7 Hz, 1H), 6.53 (s, 1H), 6.33 (d, J=1.7 Hz, 1H), 5.71 (s, 2H), 4.99 (dd, J=10.1, 2.4 Hz, 1H), 3.90 (m, 1H), 3.39 (m, 1H), 2.34 (m, 1H), 2.30 (s, 3H), 1.93 (m, 1H), 1.80 (m, 1H), 1.62-1.39 (m, 3H).

### Step 2: Preparation of 7,12-dimethyl-5,6,9,13-tetrazatricyclo[8.4.0.0²⁶]tetradeca-1(10),2,4,11,13-pentaen-8-one N77

To a solution of 2-methyl-5-(2-tetrahydropyran-2-ylpyrazol-3-yl)pyridin-4-amine (N77 1, 7.25 g, 27.8 mmol) in dry DCM (140 mL) at 0° C. were added DIPEA (9.3 mL, 55.6 mmol) and 2-bromopropionyl chloride (4.30 mL, 41.7 mmol) and the reaction mixture was stirred at RT for 1 h. Saturated aqueous NH₄Cl solution was added and the reaction mixture was extracted with EtOAc (3×). The combined organic layers were washed with brine (2×), dried over MgSO₄, filtered and concentrated to dryness. The residue was dissolved in 1,4-dioxane (280 mL), a 1 N aq. HCl solution (139 mL, 139 mmol) was added and the reaction mixture was stirred at RT for 1 h. The reaction mixture was neutralized by the addition of saturated aqueous NaHCO₃ solution and extracted with EtOAc (3×). The combined organic layers were washed with brine (2×), dried over MgSO₄, filtered and concentrated to dryness. The residue was then dissolved in dry DMF (280 mL), NaH (65% dispersion in mineral oil, 3.08 g, 83.4 mmol) was added portionwise at 0° C. and the reaction mixture was stirred at RT for 2 h. Saturated aqueous NH₄Cl solution was carefully added at 0° C. and the reaction mixture was extracted with EtOAc (3×). The combined organic layers were washed with brine (2×), dried over MgSO₄, filtered and concentrated to dryness. The crude solid was purified by column chromatography on silica gel (using a gradient of 0% to 10% MeOH in DCM as eluent). The resulting solid was then triturated in DCM, filtered and vacuum-dried to afford the title compound as a white solid (1.86 g, yield: 28%). LC-MS (Method A1) m/z [M+H]⁺: 229.1; rt: 0.52 min; purity: 95%. ¹H NMR (400 MHz, DMSO-d₆) δ 10.89 (s, 1H), 8.71 (s, 1H), 7.61 (d, J=1.9 Hz, 1H), 7.02 (s, 1H), 6.78 (d, J=1.9 Hz, 1H), 4.92 (q, J=7.0 Hz, 1H), 2.50 (s, 3H), 1.57 (d, J=7.0 Hz, 3H).

### Intermediate N78: 3-fluoro-10-methyl-4,8,12-triazatricyclo[9.4.0.0^{2,7}]pentadeca-1(11),2(7),3,5,12,14-hexaen-9-one

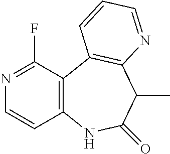 [SMILES: CC1C(=O)Nc2ccnc(F)c2-c2cccnc21]

To a solution of 4-amino-3-bromo-2-fluoropyridine (75 mg, 0.37 mmol) and ethyl 2-[3-(4,4,5,5-tetramethyl-1,3,2-dioxaborolan-2-yl)-2-pyridyl]propanoate (Intermediate N31_1, 244 mg, 0.74 mmol) in 1,4-dioxane (1.9 mL) were added K₂CO₃ (104 mg, 0.74 mmol) and water (82 μL). The reaction mixture was degassed with nitrogen for 5 min before addition of Pd[(Amphos)₂Cl]2 (CAS 887919-35-9, 14 mg, 0.02 mmol) and the reaction mixture was stirred at 100° C. for 20 h. After cooling down to RT, water was added, and the reaction mixture was extracted with EtOAc (3×). The combined organic layers were washed with brine, dried over MgSO₄, filtered and concentrated to dryness. The residue was then dissolved in dry toluene (2.4 mL) and cooled to 0° C. before slow addition of a 1.5M solution of LiHMDS in THF (0.37 mL, 0.56 mmol) and the reaction mixture was stirred at rt for 1.5 h. A saturated aqueous NH₄Cl solution was added and the reaction mixture was extracted with EtOAc (3×). The combined organic layers were washed with brine, dried over MgSO₄, filtered and concentrated to dryness. The residue was triturated in Et₂₀, filtered and vacuum-dried to afford the title compound as an off-white solid (45 mg, yield: 50%). LC-MS (Method B1) m/z [M+H]⁺: 244.1; rt: 0.89 min; purity: 96%. ¹H NMR (400 MHz, DMSO-d₆) δ 10.87 (s, 1H), 8.68 (dd, J=4.7, 1.7 Hz, 1H), 8.18 (d, J=5.5 Hz, 1H), 8.12 (ddd, J=7.9, 4.6, 1.7 Hz, 1H), 7.48 (dd, J=7.9, 4.7 Hz, 1H), 7.18 (d, J=5.5 Hz, 1H), 3.63 (q, J=6.6 Hz, 1H), 1.50 (d, J=6.6 Hz, 3H).

### Intermediates N79-128

The following intermediates lactams have been prepared following similar procedures as the ones used for the preparation of related lactames, such as Intermediates N1-N4, N6, N8-23, N25-N37, N39-N41, N43-N45, N53-N58, N59a, N59b, N60-N61, N63-N64, N65_1, N66, N68_4, N69_2, N71_2, N72_6, N73_3, N75-N78, starting either from the corresponding bromo or boronate of the amino-aryl or heteroaryl and from the corresponding boronate or bromo aryl- or heteroaryl-esters, respectively (specific intermediates referenced in the table below).

| # | Structure | Chemical name | Method and Characterization |
|---|---|---|---|
| N79 | 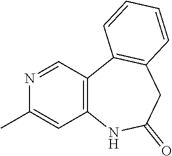 [SMILES: Cc1cc2c(cn1)-c1ccccc1CC(=O)N2] | 3-methyl-5,7- dihydropyrido[4,3- d][3]benzazepin- 6-one | Suzuki reaction using 4-amino-5-bromo- 2-methylpyridine (CAS 10460-50-1), methyl 2-(2-(4,4,5,5-tetramethyl-1,3,2- dioxaborolan-2-yl)phenyl)acetate (CAS 956229-86-0), K₂CO₃, Pd(dppf)₂ heated in H₂O/dioxane at 100° C. (34% yield). LC-MS (Method B1) m/z [M + H]⁺: 225.0; rt: 1.72 min |
| N80 | 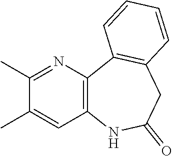 [SMILES: Cc1cc2c(nc1C)-c1ccccc1CC(=O)N2] | 2,3-dimethyl-5,7- dihydropyrido[3,2- d][3]benzazepin- 6-one | Suzuki reaction using 2-bromo-5,6- dimethylpyridin-3-amine (CAS 1824593- 49-8), methyl 2-(2-(4,4,5,5-tetramethyl- 1,3,2-dioxaborolan-2-yl)phenyl)acetate (CAS 956229-86-0), K₂CO₃, Pd(dppf)₂ heated in H₂O/dioxane at 100° C. (53% yield). LC-MS (Method B1) m/z [M + H]⁺: 239.2; rt: 2.01 min |
| N81 | 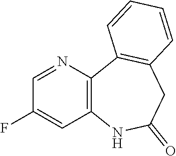 [SMILES: O=C1Cc2ccccc2-c2ncc(F)cc2N1] | 3-fluoro-5,7- dihydropyrido[3,2- d][3]benzazepin- 6-one | Suzuki reaction using 3-amino-2-bromo- 5-fluoropyridine (CAS 884495-03-8), methyl 2-(2-(4,4,5,5-tetramethyl-1,3,2- dioxaborolan-2-yl)phenyl)acetate (CAS 956229-86-0), K₂CO₃, Pd(dppf)₂ heated in H₂O/dioxane at 100° C. (53% yield). LC-MS (Method B1) m/z [M + H]⁺: 229.2; rt: 1.87 min |
| N82 | 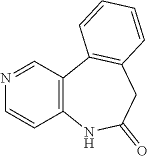 [SMILES: O=C1Cc2ccccc2-c2cnccc2N1] | 5,7- dihydropyrido[4,3- d][3]benzazepin- 6-one | Suzuki reaction using 4-amino-3- bromopyridine (CAS 13534-98-0), methyl 2-(2-(4,4,5,5-tetramethyl-1,3,2- dioxaborolan-2-yl)phenyl)acetate (CAS 956229-86-0), K₂CO₃, Pd(dppf)₂ heated in H₂O/dioxane at 100° C. (35% yield). LC-MS (Method B1) m/z [M + H]⁺: 211.0; rt: 1.62 min |
| N83 | 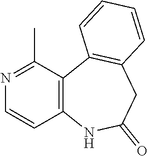 [SMILES: Cc1nccc2c1-c1ccccc1CC(=O)N2] | 1-methyl-5,7- dihydropyrido[4,3- d][3]benzazepin- 6-one | Suzuki reaction using 3-bromo-2- methylpyridin-4-amine (CAS 97944-41- 7), methyl 2-(2-(4,4,5,5-tetramethyl- 1,3,2-dioxaborolan-2-yl)phenyl)acetate (CAS 956229-86-0), Cs₂CO₃, Tetrakis(triphenylphosphine)palladium(0) heated in DME at 120° C. The crude product was further treated with LiHMDS (3 eq.) in toluene at RT (14% yield over 2 steps). LC-MS (Method B1) m/z [M + H]⁺: 225.0; rt: 1.72 min |
| N84 | 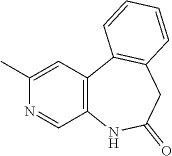 [SMILES: Cc1cc2c(cn1)NC(=O)Cc1ccccc1-2] | 2-methyl-5,7- dihydropyrido[3,4- d][3]benzazepin- 6-one | Suzuki reaction using 4-bromo-6- methylpyridin-3-amine (CAS 1060812- 94-3), methyl 2-(2-(4,4,5,5-tetramethyl- 1,3,2-dioxaborolan-2-yl)phenyl)acetate (CAS 956229-86-0), Cs₂CO₃, Pd(dppf)₂ heated in DME at 120° C. (100% yield). LC-MS (Method B1) m/z [M + H]⁺: 225.1; rt: 0.97 min |
| N85 | 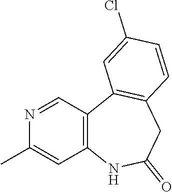 [SMILES: Cc1cc2c(cn1)-c1cc(Cl)ccc1CC(=O)N2] | 10-chloro-3- methyl-5,7- dihydropyrido[4,3- d][3]benzazepin- 6-one | Suzuki reaction using 4-amino-5-bromo- 2-methylpyridine (CAS 10460-50-1), methyl 2-[4-chloro-2-(4,4,5,5- tetramethyl-1,3,2-dioxaborolan-2- yl)phenyl]acetate (MFCD18757674), Cs₂CO₃, Pd(dppf)₂ heated in dioxane at 100° C. (51% yield). LC-MS (Method B1) m/z [M + H]⁺: 259.0; rt: 1.07 min |
| N86 | 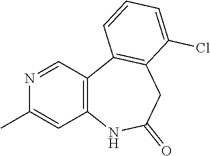 [SMILES: Cc1cc2c(cn1)-c1cccc(Cl)c1CC(=O)N2] | 8-chloro-3- methyl-5,7- dihydropyrido[4,3- d][3]benzazepin- 6-one | Suzuki reaction using 4-amino-5-bromo- 2-methylpyridine (CAS 10460-50-1), methyl 2-[2-chloro-6-(4,4,5,5- tetramethyl-1,3,2-dioxaborolan-2- yl)phenyl]acetate (MFCD18757673), Cs₂CO₃, Pd(dppf)₂ heated in dioxane at 110° C. (50% yield). LC-MS (Method B1) m/z [M + H]⁺: 259.0; rt: 1.05 min |
| N87 | 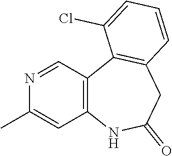 [SMILES: Cc1cc2c(cn1)-c1c(Cl)cccc1CC(=O)N2] | 11-chloro-3- methyl-5,7- dihydropyrido[4,3- d][3]benzazepin- 6-one | Suzuki reaction using 4-amino-5-bromo- 2-methylpyridine (CAS 10460-50-1), methyl 2-[3-chloro-2-(4,4,5,5- tetramethyl-1,3,2-dioxaborolan-2- yl)phenyl]acetate (MFCD18757675), K₃PO₄, Pd₂(dba)₃/2- dicyclohexylphosphino-2,6- dimethoxybiphenyl heated in toluene at 120° C. (99% yield). LC-MS (Method B1) m/z [M + H]⁺: 259.0; rt: 1.03 min |
| N88 | 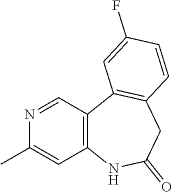 [SMILES: Cc1cc2c(cn1)-c1cc(F)ccc1CC(=O)N2] | 10-fluoro-3- methyl-5,7- dihydropyrido[4,3- d][3]benzazepin- 6-one | Suzuki reaction using 4-amino-5-bromo- 2-methylpyridine (CAS 10460-50-1), methyl 2-[4-fluoro-2-(4,4,5,5- tetramethyl-1,3,2-dioxaborolan-2- yl)phenyl]acetate (CAS 2376922-64-2), Cs₂CO₃, Pd(dppf)₂ heated in dioxane at 110° C. (71% yield). LC-MS (Method B1) m/z [M + H]⁺: 243.1; rt: 0.98 min |
| N89 | 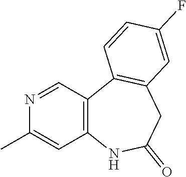 [SMILES: Cc1cc2c(cn1)-c1ccc(F)cc1CC(=O)N2] | 9-fluoro-3- methyl-5,7- dihydropyrido[4,3- d][3]benzazepin- 6-one | Suzuki reaction using 4-amino-5-bromo- 2-methylpyridine (CAS 10460-50-1), methyl 2-[5-fluoro-2-(4,4,5,5- tetramethyl-1,3,2-dioxaborolan-2- yl)phenyl]acetate (CAS 1352734-02-1), Cs₂CO₃, Pd(dppf)₂ heated in dioxane at 110° C. (64% yield). LC-MS (Method B1) m/z [M + H]⁺: 243.0; rt: 0.98 min |
| N90 | 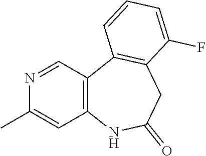 [SMILES: Cc1cc2c(cn1)-c1cccc(F)c1CC(=O)N2] | 8-fluoro-3- methyl-5,7- dihydropyrido[4,3- d][3]benzazepin- 6-one | Suzuki reaction using 4-amino-5-bromo- 2-methylpyridine (CAS 10460-50-1), methyl 2-[2-fluoro-6-(4,4,5,5- tetramethyl-1,3,2-dioxaborolan-2- yl)phenyl]acetate (MFCD18731106), Cs₂CO₃, Pd(dppf)₂ heated in dioxane at 110° C. (95% yield). LC-MS (Method B1) m/z [M + H]⁺: 243.0; rt: 0.98 min |
| N91 | 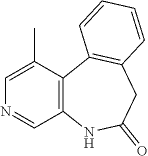 [SMILES: Cc1cncc2c1-c1ccccc1CC(=O)N2] | 1-methyl-5,7- dihydropyrido[3,4- d][3]benzazepin- 6-one | Suzuki reaction using 4-bromo-5- methylpyridin-3-amine (CAS 1806996- 46-2), methyl 2-(2-(4,4,5,5-tetramethyl- 1,3,2-dioxaborolan-2-yl)phenyl)acetate (CAS 956229-86-0), Cs₂CO₃, Pd(dppf)₂ heated in DME at 120° C. The crude product was further treated with LiHMDS (3 eq.) in toluene at RT (53% yield over 2 steps). LC-MS (Method B1) m/z [M + H]⁺: 225.1; rt: 0.99 min |
| N92 | 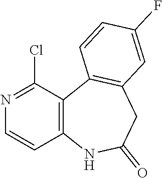 [SMILES: O=C1Cc2cc(F)ccc2-c2c(ccnc2Cl)N1] | 1-chloro-9-fluoro- 5,7- dihydropyrido[4,3- d][3]benzazepin- 6-one | Suzuki reaction using 2-chloro-3-iodo- pyridin-4-amine (CAS 909036-46-0), methyl 2-[5-fluoro-2-(4,4,5,5- tetramethyl-1,3,2-dioxaborolan-2- yl)phenyl]acetate (CAS 1352734-02-1), K₂CO₃, Pd(Amphos)₂Cl₂ heated in H₂O/dioxane at 100° C. The crude product was further treated with LiHMDS (3 eq.) in toluene at RT (63% yield over 2 steps). LC-MS (Method A1) m/z [M + H]⁺: 262.9; rt: 1.1 min |
| N93 | 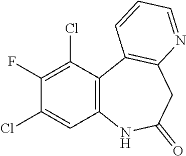 [SMILES: O=C1Cc2ncccc2-c2c(cc(Cl)c(F)c2Cl)N1] | 9,11-dichloro-10- fluoro-5,7- dihydropyrido[2,3- d][1]benzazepin- 6-one | Suzuki reaction using 2-bromo-3,5- dichloro-4-fluoroaniline (CAS 1092350- 32-7), methyl 2-[3-(4,4,5,5-tetramethyl- 1,3,2-dioxaborolan-2-yl)-2- pyridyl]acetate (MFCD18733859), K₂CO₃, Pd(Amphos)₂Cl₂ heated in H₂O/dioxane at 100° C. (19% yield). LC- MS (Method B1) m/z [M + H]⁺: 297/299; rt: 1.22 min |
| N94 | 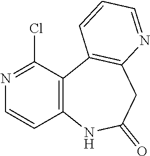 [SMILES: O=C1Cc2ncccc2-c2c(ccnc2Cl)N1] | 3-chloro-4,8,12- triazatricyclo [9.4.0.0^{2,7}]pentadeca- 1(11),2(7),3,5,12,14- hexaen-9-one | Suzuki reaction using 2-chloro-3-iodo- pyridin-4-amine (CAS 909036-46-0), methyl 2-[3-(4,4,5,5-tetramethyl-1,3,2- dioxaborolan-2-yl)-2-pyridyl]acetate (MFCD18733859), K₂CO₃, Pd(Amphos)₂Cl₂ heated in H2O/dioxane at 100° C. (3% yield). LC-MS (Method A1) m/z [M + H]⁺: 246.0; rt: 0.96 min |
| N95 | 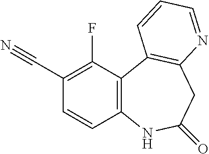 [SMILES: N#Cc1ccc2c(c1F)-c1cccnc1CC(=O)N2] | 11-fluoro-6-oxo- 5,7- dihydropyrido[2,3-d] [1]benzazepine- 10-carbonitrile | Suzuki reaction using 4-amino-3-bromo- 2-fluorobenzonitrile (CAS 2090323-74-1), methyl 2-[3-(4,4,5,5-tetramethyl- 1,3,2-dioxaborolan-2-yl)-2- pyridyl]acetate (MFCD18733859), K₂CO₃, Pd(Amphos)₂Cl₂ heated in H₂O/dioxane at 100° C. (68% yield). LC- MS (Method B1) m/z [M + H]⁺: 252.2; rt: 0.96 min |
| N96 | 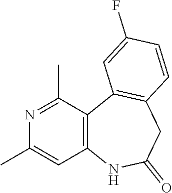 [SMILES: Cc1cc2c(c(C)n1)-c1cc(F)ccc1CC(=O)N2] | 10-fluoro-1,3- dimethyl-5,7- dihydropyrido[4,3- d][3]benzazepin- 6-one | Suzuki reaction using 3-bromo-2,6- dimethylpyridin-4-amine (CAS 33259- 24-4), methyl 2-[4-fluoro-2-(4,4,5,5- tetramethyl-1,3,2-dioxaborolan-2- yl)phenyl]acetate (CAS 2376922-64-2), K₂CO₃, Pd(Amphos)₂Cl₂ heated in H₂O/dioxane at 100° C. The crude product was further treated with LiHMDS (3 eq.) in toluene at RT (59% yield over 2 steps). LC-MS (Method B1) m/z [M + H]⁺: 257.0; rt: 1.02 min |
| N97 | 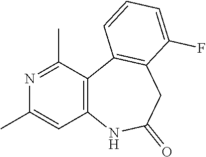 [SMILES: Cc1cc2c(c(C)n1)-c1cccc(F)c1CC(=O)N2] | 8-fluoro-1,3- dimethyl-5,7- dihydropyrido[4,3- d][3]benzazepin- 6-one | Suzuki reaction using 3-bromo-2,6- dimethylpyridin-4-amine (CAS 33259- 24-4), methyl 2-[2-fluoro-6-(4,4,5,5- tetramethyl-1,3,2-dioxaborolan-2- yl)phenyl]acetate (MFCD18731106), K₂CO₃, Pd(Amphos)₂Cl₂ heated in H₂O/dioxane at 100° C. The crude product was further treated with LiHMDS (3 eq.) in toluene at RT (62% yield over 2 steps). LC-MS (Method B1) m/z [M + H]⁺: 257.0; rt: 1.03 min |
| N98 | 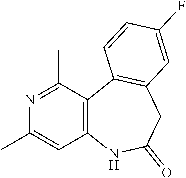 [SMILES: Cc1cc2c(c(C)n1)-c1ccc(F)cc1CC(=O)N2] | 9-fluoro-1,3- dimethyl-5,7- dihydropyrido[4,3- d][3]benzazepin- 6-one | Suzuki reaction using 3-bromo-2,6- dimethylpyridin-4-amine (CAS 33259- 24-4), methyl 2-[5-fluoro-2-(4,4,5,5- tetramethyl-1,3,2-dioxaborolan-2- yl)phenyl]acetate (CAS 1352734-02-1), K₂CO₃, Pd(Amphos)₂Cl₂ heated in H₂O/dioxane at 100° C. The crude product was further treated with LiHMDS (3 eq.) in toluene at RT (80% yield over 2 steps). LC-MS (Method B1) m/z [M + H]⁺: 257.0; rt: 1.03 min |
| N99 | 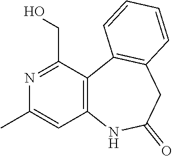 [SMILES: Cc1cc2c(c(CO)n1)-c1ccccc1CC(=O)N2] | 1- (hydroxymethyl)- 3-methyl-5,7- dihydropyrido[4,3- d][3]benzazepin- 6-one | Suzuki reaction using (4-amino-3- bromo-6-methyl-2-pyridyl)methyl acetate (Intermediate N46), methyl 2-(2- (4,4,5,5-tetramethyl-1,3,2-dioxaborolan- 2-yl)phenyl)acetate (CAS 956229-86-0), K₂CO₃, Pd(Amphos)₂Cl₂ heated in H₂O/dioxane at 100° C. The crude product was further treated with LiHMDS (3 eq.) in toluene at RT (60% yield over 2 steps). LC-MS (Method B1) m/z [M + H]⁺: 255.0; rt: 0.83 min |
| N100 | 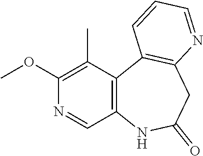 [SMILES: COc1ncc2c(c1C)-c1cccnc1CC(=O)N2] | 4-methoxy-3- methyl-5,8,12- triazatricyclo [9.4.0.0^{2,7}]pentadeca- 1(11),2(7),3,5,12,14- hexaen-9-one | Suzuki reaction using 4-bromo-6- methoxy-5-methyl-pyridin-3-amine (Intermediate N47), methyl 2-[3- (4,4,5,5-tetramethyl-1,3,2-dioxaborolan- 2-yl)-2-pyridyl]acetate (MFCD18733859), K₂CO₃, Pd(Amphos)₂Cl₂ heated in H₂O/dioxane at 100° C. (38% yield). LC-MS (Method B1) m/z [M + H]⁺: 256.2; rt: 0.96 min |
| N101 | 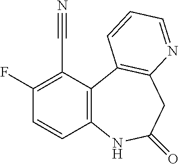 [SMILES: N#Cc1c(F)ccc2c1-c1cccnc1CC(=O)N2] | 10-fluoro-6-oxo- 5,7- dihydropyrido[2,3- d][1]benzazepine- 11-carbonitrile | Suzuki reaction using 2-bromo-3-amino- 6-fluorobenzonitrile (CAS 1415633-90-7), methyl 2-[3-(4,4,5,5-tetramethyl- 1,3,2-dioxaborolan-2-yl)-2- pyridyl]acetate (MFCD18733859), K₂CO₃, Pd(Amphos)₂Cl₂ heated in H₂O/dioxane at 100° C. (37% yield). LC- MS (Method B1) m/z [M + H]⁺: 254.1; rt: 0.93 min |
| N102 | 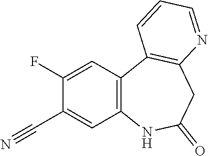 [SMILES: N#Cc1cc2c(cc1F)-c1cccnc1CC(=O)N2] | 10-fluoro-6-oxo- 5,7- dihydropyrido[2,3- d][1]benzazepine-9- carbonitrile | Suzuki reaction using 5-amino-4-bromo- 2-fluorobenzonitrile (CAS 893615-28-6), methyl 2-[3-(4,4,5,5-tetramethyl-1,3,2- dioxaborolan-2-yl)-2-pyridyl]acetate (MFCD18733859), K₂CO₃, Pd(dppf)₂ heated in H₂O/dioxane at 80° C. (48% yield). LC-MS (Method B1) m/z [M + H]⁺: 254.1; rt: 0.95 min |
| N103 | 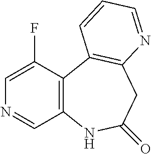 [SMILES: O=C1Cc2ncccc2-c2c(F)cncc2N1] | 3-fluoro-5,8,12- triazatricyclo [9.4.0.0^{2,7}]pentadeca- 1(11),2(7),3,5,12- 14-hexaen-9-one | Suzuki reaction using 4-bromo-5-fluoro- pyridin-3-amine; 2,2,2-trifluoroacetic acid (CAS 1805270-33-0), methyl 2-[3- (4,4,5,5-tetramethyl-1,3,2-dioxaborolan- 2-yl)-2-pyridyl]acetate (MFCD18733859), K₂CO₃, Pd(Amphos)₂Cl₂ heated in H₂O/dioxane at 100° C. (21% yield). LC-MS (Method B1) m/z [M + H]⁺: 230.1; rt: 0.77 min |
| N104 | 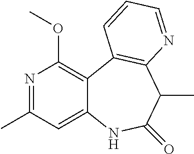 | 3-methoxy-5,10- dimethyl-4,8,12- triazatricyclo [9.4.0.0^{2,7}]pentadeca- 1(11),2,4,6,12,14- hexaen-9-one | Suzuki reaction using 3-bromo-2- methoxy-6-methyl-pyridin-4-amine (Intermediate N25_1), ethyl 2-[3- (4,4,5,5-tetramethyl-1,3,2-dioxaborolan- 2-yl)-2-pyridyl]propanoate (Intermediate N31_1), CsF, S-Phos, Pd(OAc)₂ heated in H₂O/dioxane at 90° C. The crude product was further treated with K₂CO₃ in MeOH (42% yield over 2 steps). LC- MS (Method A7) m/z [M + H]⁺: 270.2; rt: 1.55 min |
| N105 | 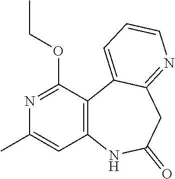 | 3-ethoxy-5- methyl-4,8,12- triazatricyclo [9.4.0.0^{2,7}]pentadeca- 1(15),2(7),3,5,11, 13-hexaen-9-one | Suzuki reaction using 3-bromo-2- ethoxy-6-methyl-pyridin-4-amine (CAS 2130998-81-9), ethyl 2-[3-(4,4,5,5- tetramethyl-1,3,2-dioxaborolan-2-yl)-2- pyridyl]acetate (Intermediate N25_2), K₂CO₃, Pd(Amphos)₂Cl₂ heated in H₂O/dioxane at 80° C. LC-MS of the non cyclized Intermediate ethyl 2-[3-(4- amino-2-ethoxy-6-methyl-3-pyridyl)-2- pyridyl]acetate (Method A7) m/z [M + H]⁺: 316.1; rt: 0.71 min. The crude Intermediate was further treated with |
| | | | K₂CO₃ in EtOH and used as such in the |
| | | | alkylation step (20% yield (crude) over |
| | | | 2 steps). |
| N107 | 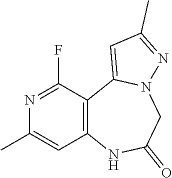 [SMILES: Cc1cc2c(c(F)n1)-c1cc(C)nn1CC(=O)N2] | 14-fluoro-4,12- dimethyl- 5,6,9,13- tetrazatricyclo [4.0.0^{2,6}]tetradeca- 1(10),2,4,11,13- pentaen-8-one | Procedure analog to method of synthesis of Intermediate N27. Suzuki reaction using 3-methyl-1- tetrahydropyran-2-yl-5-(4,4,5,5- tetramethyl-1,3,2-dioxaborolan-2- yl)pyrazole (Intermediate N30_3), 3- bromo-2-fluoro-6-methyl-pyridin-4- amine (Intermediate N131), K₃PO₄, Pd₂(dba)₃/SPhos heated in toluene at 100° C. (yield (crude) 50%) LC-MS (Method B1) m/z [M + H]⁺: 247.1; rt: 0.94 min |
| N108 | 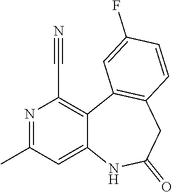 | 10-fluoro-3- methyl-6-oxo- 5,7- dihydropyrido[4,3- d][3]benzazepine-1- carbonitrile | Suzuki reaction using 3-bromo-2-chloro- 6-methyl-pyridin-4-amine (CAS 1781997-85-0), methyl 2-[4-fluoro-2- (4,4,5,5-tetramethyl-1,3,2-dioxaborolan- 2-yl)phenyl]acetate (CAS 2376922-64- 2), CsF, Pd(Amphos)₂Cl₂ heated in H₂O/dioxane at 100° C. The crude product was further treated with K₂CO₃ in EtOH (26% yield over 2 steps). LC- MS (Method A7) m/z [M + H]⁺: 277.1/ 279.1; rt: 1.75 min |
| N109 | 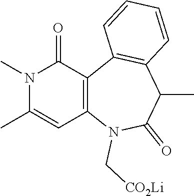 | [2-(2,3,7- trimethyl-1,6- dioxo-7H- pyrido[4,3- d][3]benzazepin-5- yl)acetyl]oxylithium | Procedure analog to the method for the synthesis of Intermediate N70, but using ethyl 2-(2-bromophenyl)acetate (CAS 2178-24-7) instead of ethyl 2-(2-bromo- 5-fluoro-phenyl)acetate (Intermediate N70_1). LC-MS (Method A7) m/z [M + 2H − Li]⁺: 327.2; rt: 1.35 min |
| N110 | 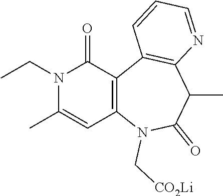 | [2-(4-ethyl-5,10- dimethyl-3,9- dioxo-4,8,12- triazatricyclo [9.4.0.0^{2,7}]pentadeca- 1(11),2(7),5,12,14- pentaen-8- yl)acetyl]oxylithium | Procedure analog to the method for the synthesis of Intermediate N70, but using ethyl 2-(3-bromo-2-pyridyl)acetate (Intermediate N3_1) instead of ethyl 2- (2-bromo-5-fluoro-phenyl)acetate (Intermediate N70_1) and iodoethane instead of iodomethane. LC-MS (Method A7) m/z [M + 2H − Li]⁺: 342.2; rt: 1.09 min |
| N111 | 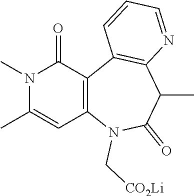 | [2-(4,5,10- trimethyl-3,9- dioxo-4,8,12- triazatricyclo [9.4.0.0^{2,7}]pentadeca- 1(11),2(7),5,12, 14-pentaen-8- yl)acetyl]oxylithium | Procedure analog to the method for the synthesis of Intermediate N70, but using ethyl 2-(3-bromo-2-pyridyl)acetate (Intermediate N3_1) instead of ethyl 2- (2-bromo-5-fluoro-phenyl)acetate (Intermediate N70_1). LC-MS (Method A7) m/z [M + 2H − Li]⁺: 328.1; rt: 0.85 min |
| N113 | 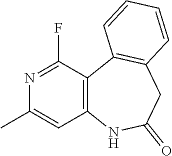 [SMILES: Cc1cc2c(c(F)n1)-c1ccccc1CC(=O)N2] | 1-fluoro-3- methyl-5,7- dihydropyrido[4,3- d][3]benzazepin- 6-one | Suzuki reaction using methyl 2-(2- bromophenyl)acetate (CAS 57486-69- 8), 2-fluoro-6-methyl-3-(4,4,5,5- tetramethyl-1,3,2-dioxaborolan-2- yl)pyridin-4-amine (Intermediate N16_1), K₂CO₃, Pd(dppf)₂ heated in H₂O/dioxane at 100° C. (35% yield). LC- MS (Method A1) m/z [M + H]⁺: 243.1; rt: 1.08 min, purity 98%. 1H NMR (400 MHz, DMSO-d₆) δ 10.60 (s, 1H), 7.64 (t, |
| | | | J = 6.3 Hz, 1H), 7.50-7.37 (m, 3H), |
| | | | 6.96 (s, 1H), 3.49 (s, 2H), 2.43 (s, 3H). |
| N114 | 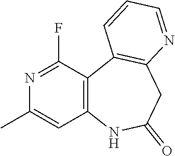 [SMILES: Cc1cc2c(c(F)n1)-c1cccnc1CC(=O)N2] | 3-fluoro-5- methyl-4,8,12- triazatricyclo [9.4.0.0^{2,7}]pentadeca- 1(11),2(7),3,5,12- 14-hexaen-9-one | Suzuki reaction using ethyl 2-(3-bromo- 2-pyridyl)acetate (Intermediate N3_1), 2-fluoro-6-methyl-3-(4,4,5,5-tetramethyl- 1,3,2-dioxaborolan-2-yl)pyridin-4-amine (Intermediate N16_1), K₂CO₃, Pd(dppf)₂ heated in H₂O/dioxane at 100° C. (26% yield). LC-MS (Method A1) m/z [M + H]⁺: 244.1; rt: 0.78 min |
| N115 | 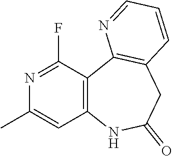 [SMILES: Cc1cc2c(c(F)n1)-c1ncccc1CC(=O)N2] | 15-fluoro-13- methyl-3,10,14- triazatricyclo [9.4.0.0^{2,7}]pentadeca- 1(11),2(7),3,5,12, 14-hexaen-9-one | Suzuki reaction using ethyl 2-(2- bromopyridin-3-yl)acetate (CAS 1824117-77-2), 2-fluoro-6-methyl-3- (4,4,5,5-tetramethyl-1,3,2-dioxaborolan- 2-yl)pyridin-4-amine (Intermediate N16_1), K₃PO₄, Pd(dba)₃/SPhos heated in toluene at 100° C. The crude product was further treated with LiHMDS (3 eq.) in toluene at RT and used as such in the alkylation step. LC- |
| | | | MS (Method B1) m/z [M + H]⁺: 244.0; rt: |
| | | | 0.81 min |
| N116 | 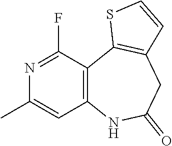 [SMILES: Cc1cc2c(c(F)n1)-c1sccc1CC(=O)N2] | 14-fluoro-12- methyl-3-thia- 9,13- diazatricyclo [8.4.0.0^{2,6}]tetra- deca-1(10),2(6),4, 11,13-pentaen-8- one | Suzuki reaction using methyl 2-(2- bromo-3-thienyl)acetate (CAS 683251- 61-8), 2-fluoro-6-methyl-3-(4,4,5,5- tetramethyl-1,3,2-dioxaborolan-2- yl)pyridin-4-amine (Intermediate N16_1), K₃PO₄, Pd₂(dba)₃/SPhos heated in toluene at 100° C. The crude product was further treated with LiHMDS (3 eq.) in toluene at RT (60% |
| | | | yield over 2 steps). LC-MS (Method A1) |
| | | | m/z [M + H]⁺: 248.9; rt: 1.09 min |
| N117 | 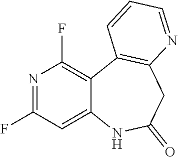 [SMILES: O=C1Cc2ncccc2-c2c(cc(F)nc2F)N1] | 3,5-difluoro- 4,8,12- triazatricyclo [9.4.0.0^{2,7}]pentadeca- 1(11),2,4,6,12,14- hexaen-9-one | Suzuki reaction using ethyl 2-(3-bromo- 2-pyridyl)acetate (Intermediate N3_1), 2,6-difluoro-3-(4,4,5,5-tetramethyl-1,3,2- dioxaborolan-2-yl)pyridin-4-amine (Intermediate N132), CsF, PEPPSI- Ipent heated in H₂O/toluene at 80° C. The crude product was further treated with LiHMDS (3 eq.) in toluene at RT (17% yield over 2 steps). LC-MS (Method A7) m/z [M + H]⁺: 248.1; rt: 1.32 min |
| N118 | 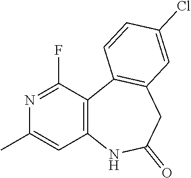 [SMILES: Cc1cc2c(c(F)n1)-c1ccc(Cl)cc1CC(=O)N2] | 9-chloro-1-fluoro- 3-methyl-5,7- dihydropyrido[4,3- d][3]benzazepin- 6-one | Suzuki reaction using methyl 2-(2- bromo-5-chloro-phenyl)acetate (CAS 455957-76-3), 2-fluoro-6-methyl-3- (4,4,5,5-tetramethyl-1,3,2-dioxaborolan- 2-yl)pyridin-4-amine (Intermediate N16_1), K₃PO₄, Pd₂(dba)₃/SPhos heated in toluene at 100° C. The crude product was further treated with LiHMDS (3 eq.) in toluene at RT (yield). LC-MS (Method B1) m/z [M − H]⁻: 275.2; rt: 1.25 min |
| N119 | 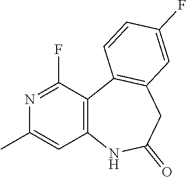 [SMILES: Cc1cc2c(c(F)n1)-c1ccc(F)cc1CC(=O)N2] | 1,9-difluoro-3- methyl-5,7- dihydropyrido[4,3- d][3]benzazepin- 6-one | Suzuki reaction using methyl 2-(2- bromo-5-fluoro-phenyl)acetate (Intermediate N70_1), 2-fluoro-6- methyl-3-(4,4,5,5-tetramethyl-1,3,2- dioxaborolan-2-yl)pyridin-4-amine (Intermediate N16_1), K₃PO₄, Pd₂(dba)₃/SPhos heated in toluene at 100° C. The crude product was further treated with LiHMDS (3 eq.) in toluene at RT (29% yield over 2 steps). LC-MS (Method A1) m/z [M + H]⁺: 261.1; rt: 1.1 min |
| N120 | 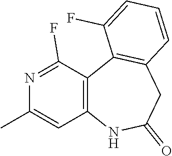 [SMILES: Cc1cc2c(c(F)n1)-c1c(F)cccc1CC(=O)N2] | 1,11-difluoro-3- methyl-5,7- dihydropyrido[4,3- d][3]benzazepin- 6-one | Suzuki reaction using methyl 2-(2- bromo-3-fluoro-phenyl)acetate (CAS 1427433-01-9), 2-fluoro-6-methyl-3- (4,4,5,5-tetramethyl-1,3,2-dioxaborolan- 2-yl)pyridin-4-amine (Intermediate N16_1), K₃PO₄, Pd₂(dba)₃/SPhos heated in toluene at 100° C. The crude product was further treated with LiHMDS (3 eq.) in toluene at RT (43% yield over 2 steps). LC-MS (Method A1) m/z [M + H]⁺: 260.9; rt: 1.1 min |
| N121 | 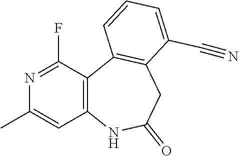 | 1-fluoro-3- methyl-6-oxo- 5,7- dihydropyrido[4,3- d][3]benzazepine- 8-carbonitrile | Suzuki reaction using methyl 2-(2- bromo-6-cyano-phenyl)acetate (CAS 1261458-69-8), 2-fluoro-6-methyl-3- (4,4,5,5-tetramethyl-1,3,2-dioxaborolan- 2-yl)pyridin-4-amine (Intermediate N16_1), K₃PO₄, Pd₂(dba)₃/SPhos heated in toluene at 100° C. The crude product was further treated with LiHMDS (3 eq.) in toluene at RT (yield). LC-MS (Method B1) m/z [M + H]⁺: 300.0; |
| | | | rt: 1.06 min |
| N122 | 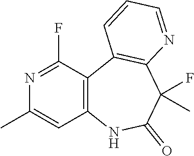 [SMILES: Cc1cc2c(c(F)n1)-c1cccnc1C(C)(F)C(=O)N2] | 3,10-difluoro- 5,10-dimethyl- 4,8,12- triazatricyclo [9.4.0.0^{2,7}]pentadeca- 1(11),2(7),3,5,12, 14-hexaen-9-one | Suzuki reaction using methyl 2-(3- bromo-2-pyridyl)-2-fluoro-propanoate (Intermediate N49), 2-fluoro-6-methyl-3- (4,4,5,5-tetramethyl-1,3,2-dioxaborolan- 2-yl)pyridin-4-amine (Intermediate N16_1), K₃PO₄, Pd(Amphos)₂Cl₂ heated in toluene at 100° C. The crude product was further treated with LiHMDS (3 eq.) in toluene at RT (21% yield over 2 steps). LC-MS (Method B1) m/z [M + H]⁺: 276.1; rt: 1.06 min |
| N123 | 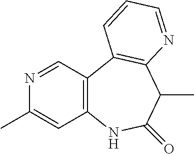 | 5,10-dimethyl- 4,8,12- triazatricyclo [9.4.0.0^{2,7}]pentadeca- 1(11),2(7),3,5,12, 14-hexaen-9-one | Suzuki reaction using ethyl 2-(3-bromo- 2-pyridyl)propanoate (Intermediate N68_2), 2-methyl-5-(tetramethyl-1,3,2- dioxaborolan-2-yl)pyridin-4-amine (CAS 1668475-78-2), K₂CO₃, Xphos Pd G3/Xphos heated in H₂O/dioxane at 100° C. (40% yield). LC-MS (Method B1) m/z [M + H]⁺: 240.1; rt: 0.81 min |
| N124 | 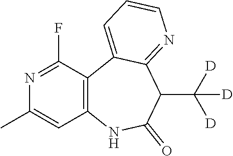 [SMILES: [2H]C([2H])([2H])C1C(=O)Nc2cc(C)nc(F)c2-c2cccnc21] | 3-fluoro-5- methyl-10- (trideuteriomethyl)- 4,8,12- triazatricyclo [9.4.0.0^{2,7}]pentadeca- 1(15),2(7),3,5,11- 13-hexaen-9-one | Suzuki reaction using ethyl 2-(3-bromo- 2-pyridyl)-3,3,3-trideuterio-propanoate (Intermediate N50), 2-fluoro-6-methyl-3- (4,4,5,5-tetramethyl-1,3,2-dioxaborolan- 2-yl)pyridin-4-amine (Intermediate N16_1), K₃PO₄, Pd₂(dba)₃/SPhos heated in toluene at 100° C. The crude product was further treated with LiHMDS (3 eq.) in toluene at RT (75% yield over 2 steps). LC-MS (Method A1) m/z [M + H]⁺: 261.3; rt: 0.92 min |
| N125 | 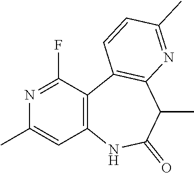 | 3-fluoro-5,10,13- trimethyl-4,8,12- triazatricyclo [9.4.0.0^{2,7}]pentadeca- 1(11),2(7),3,5,12,14- hexaen-9-one | Suzuki reaction using methyl 2-(3- bromo-6-methyl-2-pyridyl)propanoate (Intermediate N51), 2-fluoro-6-methyl-3- (4,4,5,5-tetramethyl-1,3,2-dioxaborolan- 2-yl)pyridin-4-amine (Intermediate N16_1), K₃PO₄, Pd₂(dba)₃/SPhos heated in toluene at 100° C. The crude product was further treated with LiHMDS (3 eq.) in toluene at RT (70% yield over 2 steps). LC-MS (Method A1) m/z [M + H]⁺: 272.3; rt: 0.99 min |
| N126 | 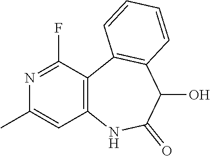 [SMILES: Cc1cc2c(c(F)n1)-c1ccccc1C(O)C(=O)N2] | 1-fluoro- hydroxy-3- methyl-5,7- dihydropyrido [4,3-d][3] benzazepin- 6-one | Suzuki reaction using methyl 2-acetoxy- 2-(2-bromophenyl)acetate (CAS 2605212-37-9), 2-fluoro-6-methyl-3- (4,4,5,5-tetramethyl-1,3,2-dioxaborolan- 2-yl)pyridin-4-amine (Intermediate N16_1), K₃PO₄, Pd₂(dba)₃/SPhos heated in toluene at 100° C. The crude product was further treated with K₂CO₃ in EtOH at 0° C. (94% yield over 2 steps). LC-MS (Method B1) m/z [M − H]⁻: 257.1 rt: 1.0 min |
| N127 | 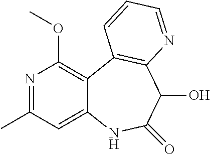 [SMILES: COc1nc(C)cc2c1-c1cccnc1C(O)C(=O)N2] | 10-hydroxy-3- methoxy-5- methyl-4,8,12- triazatricyclo [9.4.0.0^{2,7}]pentadeca- 1(11),2,4,6,12,14- hexaen-9-one | Suzuki reaction using 3-bromo-2- methoxy-6-methyl-pyridin-4-amine (Intermediate 25_1), ethyl 2-acetoxy-2- [3-(4,4,5,5-tetramethyl-1,3,2- dioxaborolan-2-yl)-2-pyridyl]acetate (Intermediate N133), CsF, S-Phos, Pd(OAc)₂ heated in dioxane at 90° C. The crude product was further treated with LiHMDS (3 eq.) in toluene at RT (13% yield over 2 steps). LC-MS (Method B5) m/z [M + H]⁺: 272.1; rt: 1.34 min |
| N128 | 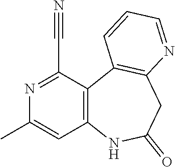 [SMILES: Cc1cc2c(c(C#N)n1)-c1cccnc1CC(=O)N2] | 5-methyl-9-oxo- 4,8,12- triazatricyclo [9.4.0.0^{2,7}]pentadeca- 1(11),2(7),3,5,12, 14-hexaene-3- carbonitrile | Suzuki reaction using 4-amino-3-bromo- 6-methyl-pyridine-2-carbonitrile (Intermediate N52), ethyl 2-[3-(4,4,5,5- tetramethyl-1,3,2-dioxaborolan-2-yl)-2- pyridyl]acetate (Intermediate N25_2), K₂CO₃, Pd(Amphos)₂Cl₂ heated in H₂O/dioxane at 100° C. The crude product was further treated with LiHMDS (3 eq.) in toluene at RT (80% yield over 2 steps). LC-MS (Method A1) m/z [M + H]⁺: 251.3; rt: 0.81 min |

### Intermediate N129: 2-chloro-N-(4-cyclopropylphenyl)acetamide

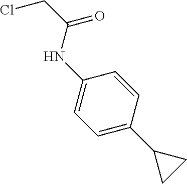 [SMILES: O=C(CCl)Nc1ccc(C2CC2)cc1]

N,N-Diisopropylethylamine (260 μL, 1.57 mmol) was added to a suspension of 4-cyclopropylaniline (70 mg, 0.53 mmol) in DCM (2.6 mL). The reaction mixture was cooled to 0° C. and chloroacethyl chloride (62 μL, 0.78 mmol) was added dropwise and the reaction mixture was stirred at RT for 4 h. Water (5 mL) was then added and the organic layer was washed with an aq. solution of HCl (1 N). The organic layer was dried over MgSO₄ and concentrated under vacuum to yield a brown solid. Trituration in diisopropyl ether under sonication afforded the title compound as a light brown solid which was used in the next step without further purification (110 mg, yield: 73%). LC-MS (Method B1) m/z [M+H]⁺: no MS response, rt: 1.24 min. ¹H NMR (400 MHz, DMSO-d₆) δ 10.18 (s, 1H), 7.45 (d, J=8.5 Hz, 2H), 7.03 (d, J=8.5 Hz, 2H), 4.22 (s, 2H), 1.87 (tt, J=8.3, 5.0 Hz, 1H), 0.97-0.84 (m, 2H), 0.68-0.57 (m, 2H).

### Intermediate N130: 3,9,10-trimethyl-5,7-dihydropyrido[4,3-d][1]benzazepin-6-one

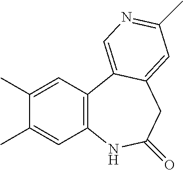

### Step 1: Preparation of ethyl 2-(5-bromo-2-fluoro-4-pyridyl)acetate N130_1

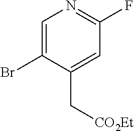 [SMILES: CCOC(=O)Cc1cc(F)ncc1Br]

To a solution of LiHMDS (1 M in hexane, 94.7 mL, 94.7 mmol) in THF (175 mL) was added 5-bromo-2-fluoro-4-methyl-pyridine (6.00 g, 31.6 mmol) under nitrogen atmosphere and the reaction mixture was stirred at RT for 2.5 h. Diethyl carbonate (6.12 mL, 50.5 mmol) was added dropwise and the reaction mixture was stirred at RT for 16 h. The reaction mixture was concentrated under vacuum. The residue was dissolved in EtOAc (150 mL) and H₂O (100 mL) was added. The aqueous layer was separated and extracted with EtOAc (2×75 mL). The combined organic layer was washed with brine (75 mL), dried over anhydrous Na₂SO₄ and concentrated under vacuum. The crude solid was purified by column chromatography on silica gel (using 10% EtOAc in hexanes as eluent) to afford the title intermediate N130_1 as a colorless oil (5.00 g, yield: 54%). LC-MS m/z [M+H]⁺: 263.8, rt: 2.30 min, purity: 89%. ¹H NMR (400 MHz, CDCl₃) δ 1.30 (t, J=7.1 Hz, 3H) 3.80 (s, 2H) 4.23 (q, J=6.9 Hz, 2H) 6.97 (d, J=2.0 Hz, 1H) 8.34 (s, 1H).

### Step 2: Preparation of ethyl 2-[5-(4,5-dimethyl-2-nitro-phenyl)-2-fluoro-4-pyridyl]acetate N130_2

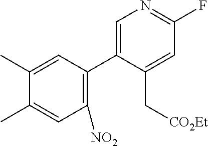 [SMILES: CCOC(=O)Cc1cc(F)ncc1-c1cc(C)c(C)cc1[N+](=O)[O-]]

To a solution of ethyl 2-(5-bromo-2-fluoro-4-pyridyl)acetate N130_1 (2.00 g, 7.63 mmol) in dioxane (30 mL) was added 2-(4,5-dimethyl-2-nitrophenyl)-4,4,5,5-tetramethyl-1,3,2-dioxaborolane N1_2 (2.33 g, 8.39 mmol), K₃PO₄ (4.05 g, 19.1 mmol) and the reaction mixture was purged with argon for 20 min. PdCl₂(dppf)₂ (0.56 g, 0.76 mmol) was added at RT and the reaction mixture was heated at 80° C. for 16 h. The reaction mixture was cooled to RT and concentrated under vacuum. The crude solid was purified by column chromatography on silica gel (using a gradient of 15% EtOAc in hexanes as eluent) to afford the title intermediate N130_2 as a brown oil (0.90 g, yield: 32%). LC-MS m/z [M+H]⁺: 333.0, rt: 2.14 min, purity: 88.8%. ¹H NMR (400 MHz, DMSO-d₆) δ 1.03 (t, J=7.2 Hz, 3H) 2.32 (s, 3H) 2.37 (s, 3H) 3.39-3.43 (m, 1H) 3.57-3.63 (m, 1H) 3.91 (q, J=7.2 Hz, 2H) 7.16 (s, 1H) 7.28 (s, 1H) 8.03 (d, J=2.0 Hz, 2H).

### Step 3: Preparation of 3-fluoro-9,10-dimethyl-5,7-dihydropyrido[4,3-d][1]benzazepin-6-one N130_3

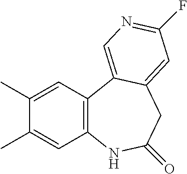 [SMILES: Cc1cc2c(cc1C)-c1cnc(F)cc1CC(=O)N2]

To a solution of ethyl 2-[5-(4,5-dimethyl-2-nitro-phenyl)-2-fluoro-4-pyridyl]acetate N130_2 (0.90 g, 2.41 mmol) in MeOH (15 mL) was added 10% Pd/C (0.15 g) at RT under nitrogen atmosphere and the reaction mixture was stirred at room temperature for 4 h under hydrogen pressure. The reaction mixture was filtered through the pad of Celite®, washed with MeOH (10 mL) and the filtrate was concentrated under vacuum to afford a mixture of 3-fluoro-9,10-dimethyl-5,7-dihydropyrido[4,3-d][1]benzazepin-6-one N130_3 and ethyl 2-[5-(2-amino-4,5-dimethyl-phenyl)-2-fluoro-4-pyridyl]acetate as a white solid (0.80 g). This mixture was used as such for the next reaction without further purification. LC-MS m/z [M+H]⁺: 256.9 and 302.9, rt: 1.64 min and 1.84 min, (44/66 ratio), purity: 40% and 52% respectively.

To a solution of the mixture of 3-fluoro-9,10-dimethyl-5,7-dihydropyrido[4,3-d][1]benzazepin-6-one N130_3 and ethyl 2-[5-(2-amino-4,5-dimethyl-phenyl)-2-fluoro-4-pyridyl]acetate (0.80 g, 2.65 mmol) in EtOH (20 mL) was added K₂CO₃ (1.10 g, 7.94 mmol) and the reaction mixture was stirred at RT for 18 h. After completion, the reaction mixture was treated with H₂O (20 mL) and the resulting precipitate was collected by filtration, washed with H₂O (50 mL) and dried under vacuum. The crude solid was purified by column chromatography on silica gel (using neat DCM as eluent) to afford the title intermediate N130_3 as a white solid (0.50 g). LC-MS m/z [M+H]⁺: 257.0, rt: 2.23 min, purity: 97%. ¹H NMR (400 MHz, DMSO-d₆) δ 2.26 (s, 3H) 2.28 (s, 3H) 3.48-3.53 (m, 2H) 6.99 (s, 1H) 7.32 (s, 1H) 7.49 (s, 1H) 8.43 (s, 1H) 10.09 (s, 1H).

### Step 4: Preparation of 3-methoxy-9,10-dimethyl-5,7-dihydropyrido[4,3-d][1]benzazepin-6-one N130_4

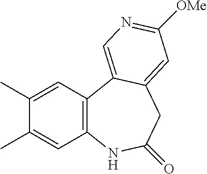 [SMILES: COc1cc2c(cn1)-c1cc(C)c(C)cc1NC(=O)C2]

To a suspension of K₂CO₃ (0.37 g, 2.65 mmol) in MeOH (5 mL) was added 3-fluoro-9,10-dimethyl-5,7-dihydropyrido[4,3-d][1]benzazepin-6-one N130_3 (0.35 g, 1.32 mmol) and the reaction mixture was heated at 65° C. for 16 h. The reaction mixture was treated with H₂O (20 mL) and the resulting precipitate was collected by filtration, washed with H₂O (50 mL) and dried under vacuum. The crude solid was purified by column chromatography on silica gel (using neat DCM as eluent) to afford the title intermediate N130_4 as an off-white solid (0.27 g, yield: 74%). LC-MS m/z [M+H]⁺: 269.0, rt: 2.44 min, purity: 98%. ¹H NMR (400 MHz, DMSO-d₆) δ 2.25 (s, 3H) 2.27 (s, 3H) 3.39 (s, 2H) 3.89 (s, 3H) 6.88 (s, 1H) 6.96 (s, 1H) 7.42 (s, 1H) 8.35 (s, 1H) 9.96 (s, 1H).

### Step 5: Preparation of 3-hydroxy-9,10-dimethyl-5,7-dihydropyrido[4,3-d][1]benzazepin-6-one N130_5

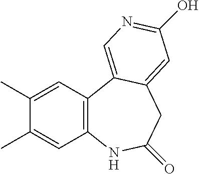 [SMILES: Cc1cc2c(cc1C)-c1cnc(O)cc1CC(=O)N2]

A stirred solution of 3-methoxy-9,10-dimethyl-5,7-dihydropyrido[4,3-d][1]benzazepin-6-one N130_4 (0.80 g, 2.89 mmol) in 33% HBr in acetic acid (12.6 mL, 211 mmol) was heated at 80° C. for 5 h. The reaction mixture was concentrated under vacuum. The crude solid was purified by triturating with Et₂O (20 mL) to afford the title intermediate N130_5 as a yellow solid (0.60 g, yield: 71%). LC-MS m/z [M+H]⁺: 255.0, rt: 1.46 min, purity: 87%. ¹H NMR (400 MHz, CDCl₃) δ 2.22 (s, 3H) 2.24 (s, 3H) 3.41 (s, 2H) 6.51 (s, 1H) 6.91 (s, 1H) 7.32 (s, 1H) 7.75 (s, 1H) 9.96 (brs, 1H) (1 exchangeable H not seen due to moisture in the solvent).

### Step 6: Preparation of (9,10-dimethyl-6-oxo-5,7-dihydropyrido[4,3-d][1]benzazepin-3-yl) trifluoromethanesulfonate N130_6

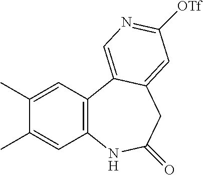 [SMILES: Cc1cc2c(cc1C)-c1cnc(OS(=O)(=O)C(F)(F)F)cc1CC(=O)N2]

To a solution of 3-hydroxy-9,10-dimethyl-5,7-dihydropyrido[4,3-d][1]benzazepin-6-one N130_5 (0.60 g, 2.29 mmol) in DCM (25 mL) were added TEA (1.60 mL, 11.4 mmol), DMAP (0.03 g, 0.23 mmol) and N-phenyl-bis(trifluoromethane)sulfonimide (1.23 g, 3.43 mmol) at RT and the reaction mixture was stirred at RT for 16 h. The reaction mixture was treated with H₂O (50 mL) and the resulting precipitate was collected by filtration, washed with H₂O (50 mL) and dried under vacuum.

The crude solid was purified by column chromatography on silica gel (using a gradient of 10 to 15% EtOAc in hexanes as eluent) to afford the title intermediate N130_6 as an off-white solid (0.38 g, yield: 43%). LC-MS m/z [M+H]⁺: 387.1, rt: 1.42 min, purity: 89%. ¹H NMR (400 MHz, CDCl₃) δ 2.28 (s, 3H) 2.29 (s, 3H) 3.53 (brs, 2H) 7.03 (s, 1H) 7.56 (s, 1H) 7.77 (s, 1H) 8.67 (s, 1H) 10.19 (s, 1H).

### Step 7: Preparation of 3,9,10-trimethyl-5,7-dihydropyrido[4,3-d][1]benzazepin-6-one N130

To a solution of (9,10-dimethyl-6-oxo-5,7-dihydropyrido[4,3-d][1]benzazepin-3-yl) trifluoromethanesulfonate N130_6 (0.10 g, 0.25 mmol) in dioxane (2 mL) were added K₂CO₃ (0.07 g, 0.50 mmol) and trimethylboroxine (0.05 mL, 0.38 mmol) at RT and the reaction mixture was purged with argon for 30 min. Xphos Pd G₃ (CAS 1445085-55-1, 0.01 g, 0.01 mmol) was added at RT and the reaction mixture was heated at 100° C. for 16 h. The reaction mixture was concentrated under vacuum and the residue was purified by column chromatography on silica gel (using a gradient of 2 to 3% MeOH in DCM as eluent) to afford the title intermediate N130 (0.03 g, yield: 47%). The reaction was repeated on 0.25 g and the products obtained from the two reactions were combined, stirred in Et₂O (2 mL) at RT, filtered and dried under vacuum to afford the title product N130 as a white solid (0.081 g, yield: 47%). LC-MS m/z [M+H]⁺: 253.0, rt: 1.47 min, purity: 99%. ¹H NMR (400 MHz, DMSO-d₆) δ 2.24 (s, 3H), 2.27 (s, 3H), 3.35 (s, 2H), 6.98 (s, 1H), 7.26 (s, 1H), 7.44 (s, 1H), 8.57 (s, 1H) (3H's merged in solvent peak+1 exchangeable H not seen due to moisture in the solvent).

### Intermediate N131: 3-bromo-2-fluoro-6-methyl-pyridin-4-amine

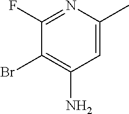

N-bromosuccinimide (7.05 g, 39.6 mmol) was added to a solution of 2-fluoro-6-methyl-pyridin-4-amine (intermediate NN445, 5.0 g, 39.6 mmol) in CH₃CN (198 mL) at RT. The reaction mixture was stirred at RT for 20 h. Water (400 mL) was added and the reaction mixture was extracted with EtOAc (300 mL). The combined organic layer was washed with brine (300 mL), dried over Na₂SO₄ and concentrated under vacuum. The residue (8.4 g) was purified by SFC (10% MeOH in C02, SiO₂, 50×341 mm, 10 mm). Two fractions were separated, collected and evaporated to afford the title intermediate N131 (second eluting isomer, 3.2 g, 40% yield) and its isomer 3-bromo-6-fluoro-2-methyl-pyridin-4-amine (first eluting isomer (4.2 g, 52% yield). ¹H NMR (400 MHz, DMSO-d₆) δ 2.17 (s, 3H), 6.43 (s, 1H), 6.60 (bs, 2H).

### Intermediate N132: 2,6-difluoro-3-(4,4,5,5-tetramethyl-1,3,2-dioxaborolan-2-yl)pyridin-4-amine

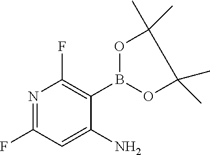 [SMILES: CC1(C)OB(c2c(N)cc(F)nc2F)OC1(C)C]

In a 60 mL vial, under N₂ atmosphere, a suspension of 4,4′-di-tert-butyl-2,2′-dipyridyl (63 mg, 0.23 mmol, 0.03 eq.) and (1,5-cyclooctadiene)(methoxy)iridium(I) dimer (76 mg, 0.115 mmol, 0.015 eq.) in dry THF (1.2 mL) was added to a solution of 2,6-difluoropyridin-4-amine (1.0 g, 7.740 mmol) and bis(pinacolato)diboron (3.9 g, 15 mmol, 2.0 eq.) in dry THF (5.8 mL). The reaction mixture was flushed with N₂ and stirred at 80° C. for 20 h. The reaction mixture was slowly poured into MeOH (50 mL) then concentrated to dryness. The brown residue was purified by column chromatography on silica gel (using a gradient of 0% to 30% EtOAc in DCM as eluent) to afford the title Intermediate N132 as a white solid (1.73 g, yield: 71%). LC-MS (Method B1) m/z: [M+H]⁺: 257.3, rt: 1.15 min, purity: 81%. ¹H NMR (400 MHz, DMSO-d₆) δ 1.29 (s, 12H), 6.10 (s, 1H), 6.87 (bs, 2H).

### Intermediate N133: ethyl 2-acetoxy-2-[3-(4,4,5,5-tetramethyl-1,3,2-dioxaborolan-2-yl)-2-pyridyl]acetate

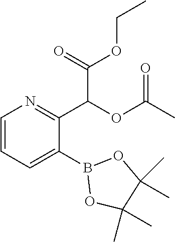 [SMILES: CCOC(=O)C(OC(C)=O)c1ncccc1B1OC(C)(C)C(C)(C)O1]

To a solution of ethyl 2-acetoxy-2-(3-bromo-2-pyridyl)acetate (Intermediate N22_1, 240 mg, 0.79 mmol) in dry 1,4-dioxane (0.6 mL) under N₂ atmosphere were added at RT bis(pinacolato)diboron (245 mg, 0.97 mmol) and potassium acetate (240 mg, 2.42 mmol). The reaction mixture was flushed with N2, then [1,1′-bis(diphenylphosphino)ferrocene]dichloropalladium(II) (29 mg, 0.039 mmol) was added. The reaction mixture was stirred at 100° C. for 3 h. The reaction mixture was diluted with EtOAc and water, then filtered over a PTFE filter and concentrated to dryness. The residue was purified by flash chromatography on silica gel (using a gradient of EtOAc in DCM from 100% to 50% as eluent) to afford the title Intermediate N133 as a brown oil (100 mg, yield: 36%). LC-MS (Method B1) m/z: [M+H]⁺: 350.1, rt: 1.37 min, purity: 92%. ¹H NMR (400 MHz, CDCl₃) δ 8.64 (dd, J=4.9, 2.0 Hz, 1H), 8.15 (dd, J=7.5, 2.0 Hz, 1H), 7.32-7.25 (m, 1H), 6.85 (s, 1H), 3.85-4.50 (m, 2H), 2.16 (s, 3H), 1.30-1.37 (m, 12H), 1.24 (t, J=7.1 Hz, 3H).

The following intermediates NN193 and NN194 have been prepared following similar procedures as the one for the preparation of related chloro acetamides such as for intermediate N129, starting from the corresponding anilines or amino-pyridines (specific intermediates referenced in the table below).

| | Structure | | |
|---|---|---|---|
| Ex | Example | Example Name | Method and characterization |
| NN193 | 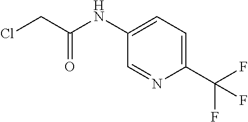 [SMILES: O=C(CCl)Nc1ccc(C(F)(F)F)nc1] | 2-chloro-N-[6- (trifluoromethyl)-3- pyridyl]acetamide | Starting from 6-(trifluoromethyl)pyridin-3- amine. ¹H NMR (400 MHz, DMSO-d6) δ 10.91 (s, 1H), 8.87 (s, 1H), 8.32 (d, J = 2.3 Hz, 1H), 7.87 (d, J = 8.6 Hz, 1H), 4.35 (s, 2H). |
| NN194 | 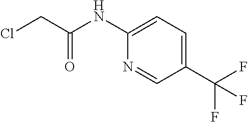 [SMILES: O=C(CCl)Nc1ccc(C(F)(F)F)cn1] | 1,3,9-trifluoro-5,7- dihydropyrido[4,3- d][3]benzazepin-6- one | Starting from 5-(trifluoromethyl)pyridin-2- amine. ¹H NMR (400 MHz, DMSO-d6) δ 11.28 (s, 1H), 8.77-8.69 (m, 1H), 8.28- 8.16 (m, 2H), 4.40 (s, 2H). |

### Intermediate NN240: 2-chloro-N-(4-(difluoromethyl)-3-fluorophenyl)acetamide

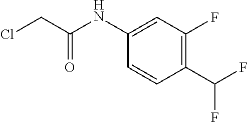 [SMILES: O=C(CCl)Nc1ccc(C(F)F)c(F)c1]

### Step 1: Synthesis of 1-(difluoromethyl)-2-fluoro-4-nitrobenzene NN240_1

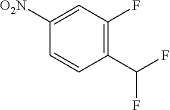 [SMILES: O=[N+]([O-])c1ccc(C(F)F)c(F)c1]

To a solution of 2-fluoro-4-nitro-benzaldehyde (2.50 g, 14.8 mmol) in DCM (30 mL) was added DAST (5.24 mL, 32.5 mmol) at 0° C. and the reaction mixture was warmed at room temperature and stirred for 1h. Progress of the reaction was monitored by TLC and LCMS. After completion, the reaction mixture was quenched with aqueous saturated NaHCO₃ (50 mL) and diluted with DCM (50 mL). The aqueous layer was separated and extracted with DCM (50 mL). The combined organic layers were dried over anhydrous Na₂SO₄ and concentrated under vacuo to afford 1-(difluoromethyl)-2-fluoro-4-nitrobenzene (2.20 g, 78%) as a colorless oil. This compound was used as such for the next reaction without further purification. ¹H NMR (400 MHz, DMSO-d₆) δ 7.35 (t, J=54 Hz, 1H) 7.95 (t, J=7.6 Hz, 1H) 8.21 (d, J=8.0 Hz, 1H) 8.30 (d, J=9.6 Hz, 1H).

### Step 2: Synthesis of 4-(difluoromethyl)-3-fluoroaniline NN240_2

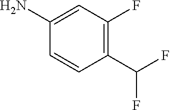 [SMILES: Nc1ccc(C(F)F)c(F)c1]

To a solution of 1-(difluoromethyl)-2-fluoro-4-nitrobenzene NN240_1 (0.50 g, 2.62 mmol) in MeOH (20 mL) was added NaBH₄ (0.25 g, 6.54 mmol) and NiCl₂ (0.31 g, 1.31 mmol) at room temperature and the reaction mixture was stirred at room temperature for 10 min. Progress of the reaction was monitored by TLC and LCMS. After completion, the reaction mixture was diluted with DCM (50 mL) and quenched with H₂O (100 mL). The aqueous layer was separated and extracted with DCM (20 mL). The combined organic layers were dried over anhydrous Na₂SO₄ and concentrated under vacuo to afford 4-(difluoromethyl)-3-fluoroaniline (0.40 g, 95%) as an off-white solid. This compound was used as such for the next reaction without further purification. ¹H NMR (400 MHz, DMSO-d₆) δ 5.88 (s, 2H) 6.33-6.42 (m, 2H) 6.91 (t, J=55.6 Hz, 1H) 7.19 (t, J=8.4 Hz, 1H).

### Step 3: Synthesis of 2-chloro-N-(4-(difluoromethyl)-3-fluorophenyl)acetamide NN240

To a solution of 4-(difluoromethyl)-3-fluoroaniline NN240_2 (0.40 g, 2.48 mmol) in DCM (20 mL) was added TEA (1.04 mL, 7.45 mmol) followed by addition of chloroacetyl chloride (0.38 mL, 4.97 mmol) at 0° C. and the reaction mixture was stirred at room temperature for 2h. Progress of the reaction was monitored by TLC and LCMS. After completion, the reaction mixture was quenched with aqueous NaHCO₃ (20 mL) and diluted with DCM (30 mL). The aqueous layer was separated and extracted with DCM (20 mL). The combined organic layers were dried over anhydrous Na₂SO₄ and concentrated under vacuo. The crude obtained was purified by column chromatography (silica, 100-200 mesh, 20% EtOAc in hexanes) to afford 2-chloro-N-(4-(difluoromethyl)-3-fluorophenyl)acetamide (0.30 g, 51%) as a white solid. LC-MS (ESI) m/e [M−H]⁺: 235.9/98.3%.

¹H NMR (400 MHz, DMSO-d₆) δ 4.30 (s, 2H) 7.14 (t, J=54.8 Hz, 1H) 7.42 (d, J=8.4 Hz, 1H) 7.61 (t, J=8.4 Hz, 1H) 7.70 (d, J=13.2 Hz, 1H) 10.72 (s, 1H).

### Intermediate NN241: 2-chloro-N-(4-(1,1-difluoroethyl)-3-fluorophenyl)acetamide

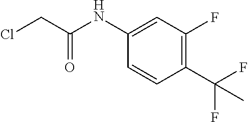 [SMILES: CC(F)(F)c1ccc(NC(=O)CCl)cc1F]

The title compound was prepared according to the same sequence of reactions as for intermediate NN240 starting from 1-(2-fluoro-4-nitrophenyl)ethan-1-one (CAS: 866579-96-6) to afford the title compound as an off-white solid. LC-MS (ESI) m/e [M−H]⁺: 249, purity: 98.4%. ¹H NMR (400 MHz, DMSO-d₆) δ 1.98 (t, J=19.2 Hz, 3H) 4.29 (s, 2H) 7.39 (d, J=8.8 Hz, 1H) 7.55 (t, J=8.4 Hz, 1H) 7.68 (d, J=13.2 Hz, 1H) 10.64 (s, 1H).

### Intermediate NN141: 4-(1-fluorocyclopropyl)aniline hydrochloride

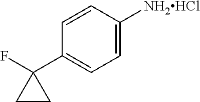

### Step 1: Synthesis of tert-butyl N-(4-bromophenyl)carbamate NN138

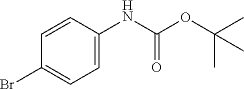

A mixture of 4-bromoaniline (3.0 g, 17.265 mmol), di-tert-butyl dicarbonate (3.9 g, 17.0 mmol) and cupric tetrafluoroborate hydrate (441.0 mg, 1.728 mmol) was stirred at room temperature for 16 h. The reaction mixture was diluted with EtOAc (80 mL), washed with water (2×30 mL), dried over Na₂SO₄, filtered and concentrated to dryness. The residue was purified by flash chromatography on silica gel (using a gradient of 0 to 20% EtOAc:Heptane as eluent) to afford the title compound as a brown solid (4.1 g, yield: 87%). LC-MS m/z [M+H-tBu]⁺: 216.0/218.0; purity: 99%. ¹H NMR (400 MHz, CDCl₃) δ 7.43-7.34 (m, 2H), 7.29-7.19 (m, 2H), 6.45 (bs., 1H), 1.51 (d, J=1.2 Hz, 9H).

### Step 2: Synthesis of tert-butyl N-[4-[1-(4,4,5,5-tetramethyl-1,3,2-dioxaborolan-2-yl)cyclopropyl]phenyl]carbamate NN139

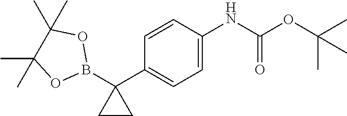 [SMILES: CC(C)(C)OC(=O)Nc1ccc(C2(B3OC(C)(C)C(C)(C)O3)CC2)cc1]

A 300 mL Schlenk flask was charged with tert-butyl N-(4-bromophenyl)carbamate NN138 (3.5 g, 12 mmol), 2,2′-Cyclopropylidenebis(4,4,5,5-tetramethyl-1,3,2-dioxaborolane) (1.8 g, 6.1 mmol), Cs₂CO₃ (6.0 g, 18 mmol), 1,4-dioxane (140 mL) and water (14 mL). The flask was evacuated and backfilled with N₂ (3 times) before addition of palladium(II) acetate (140.0 mg, 0.624 mmol) and butyldi-1-adamantylphosphine (460.0 mg, 1.283 mmol). The flask was evacuated and backfilled with N₂ (3 times) before heating at 100° C. for 16 h. The reaction mixture was diluted with EtOAc (50 mL) and water (80 mL), and filtered over a pad of Celite. The phases were then separated and the aqueous layer was extracted with EtOAc (2×30 mL). The combined organics were washed with brine (30 mL), dried over MgSO₄, filtered and concentrated to dryness. The residue was purified by flash chromatography on silica gel (using a gradient of 0 to 30% EtOAc:Heptane as eluent) to afford the title compound as a yellowish solid (1.13 g, yield: 50%). LC-MS m/z [M+H-tBu]⁺: 304.2; purity: 98%. ¹H NMR (400 MHz, DMSO-d₆) δ 9.16 (bs., 1H), 7.26 (d, J=8.2 Hz, 2H), 7.10-7.00 (m, 2H), 1.46 (s, 9H), 1.15 (s, 12H), 0.94 (m, 2H), 0.79 (m, 2H).

### Step 3: Synthesis of tert-butyl N-[4-(1-fluorocyclopropyl)phenyl]carbamate NN140

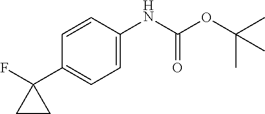 [SMILES: CC(C)(C)OC(=O)Nc1ccc(C2(F)CC2)cc1]

A flask charged with tert-butyl N-[4-[1-(4,4,5,5-tetramethyl-1,3,2-dioxaborolan-2-yl)cyclopropyl]phenyl]carbamate NN139 (1.13 g, 3.05 mmol), silver nitrate (104.0 mg, 0.61 mmol) and 1-chloromethyl-4-fluoro-1,4-diazoniabicyclo[2.2.2]octane bis(tetrafluoroborate) (3.31 g, 9.15 mmol) was evacuated and filled with N₂ (3 times). DCM (15.0 mL) was added followed by water (15.0 mL) and TFA (916.0 μL, 12.2 mmol). The resulting mixture was stirred at 50° C. for 7 h. The reaction mixture was diluted with EtOAc (80 mL) and water (30 mL). The insolubles were filtered-off over a fritted glass. The two layers were separated and the aqueous layer was extracted with EtOAc (2×15 mL). The combined organics were washed with brine (40 mL), dried over MgSO₄, filtered and concentrated to dryness. The residue was purified by preparative HPLC (Method P_B) followed by flash chromatography on silica gel (using a gradient of 0 to 10% EtOAc:Heptane as eluent) to afford the title compound as a white solid (387 mg, yield: 50%). LC-MS m/z [M+H-tBu]⁺: 196.1; purity: 99%. ¹H NMR (400 MHz, DMSO-d₆) δ 9.40 (s, 1H), 7.46 (d, J=8.5 Hz, 2H), 7.20 (d, J=8.5 Hz, 2H), 1.47 (s, 9H), 1.43-1.33 (m, 2H), 1.09-1.00 (m, 2H). ¹⁹F NMR (376 MHz, DMSO-d₆) δ−175.89 (p, J=9.5 Hz).

### Step 4: Synthesis of 4-(1-fluorocyclopropyl)aniline hydrochloride NN141

To a solution of tert-butyl N-[4-(1-fluorocyclopropyl)phenyl]carbamate NN140 (568.0 mg, 2.238 mmol) in dry DCM (23.0 mL) was added hydrochloric acid (4N in 1,4-Dioxane, 12.0 mL, 48 mmol) and the reaction mixture was stirred at room temperature for 2 h. The reaction mixture was concentrated to dryness. The residue was triturated with Et₂O (5 mL). The solid was filtered and washed with cold Et₂O (4×3 mL). The solid residue was dried under high vacuum to afford the title compound as a white solid (392.8 mg, yield: 93%). Not stable in LC-MS. ¹H NMR (400 MHz, DMSO-d₆) δ 9.64 (bs., 2H), 7.34 (d, J=8.3 Hz, 2H), 7.25 (d, J=8.3 Hz, 2H), 1.53-1.40 (m, 2H), 1.18-1.09 (m, 2H). ¹⁹F NMR (376 MHz, DMSO-d₆) δ−177.43-177.77 (m).

### Intermediate NN159: 3-fluoro-4-(1-fluorocyclopropyl)aniline hydrochloride

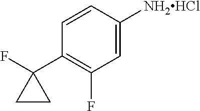

### Step 1: Synthesis of tert-butyl N-(4-bromo-3-fluoro-phenyl)carbamate NN156

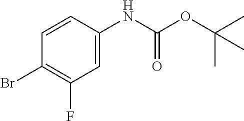 [SMILES: CC(C)(C)OC(=O)Nc1ccc(Br)c(F)c1]

A black mixture of 4-bromo-3-fluoroaniline (500.0 mg, 2.553 mmol), di-tert-butyl dicarbonate (574.0 mg, 2.551 mmol) and cupric tetrafluoroborate hydrate (65.0 mg, 0.2547 mmol) was stirred at room temperature for 16 h. The reaction mixture was diluted with DCM (50.0 mL) and water (50.0 mL). The aqueous layer was extracted with (2×50 mL), dried over MgSO₄, filtered and concentrated to dryness. The residue was purified by flash chromatography on silica gel (using a gradient of 0 to 20% EtOAc:Heptane as eluent) to afford the title compound as a brownish solid (629 mg, yield: 85%). LC-MS m/z [M+H]*: No mass response; purity: 100%. ¹H NMR (400 MHz, DMSO-d₆) δ 9.74 (s, 1H), 7.60-7.47 (m, 2H), 7.19 (dd, J=8.8, 2.4 Hz, 1H), 1.47 (s, 9H). ¹⁹F NMR (376 MHz, DMSO-de) 6-107.32 (dd, J=11.6, 8.2 Hz).

### Step 2: Synthesis of tert-butyl N-[3-fluoro-4-[1-(4,4,5,5-tetramethyl-1,3,2-dioxaborolan-2-yl)cyclopropyl]phenyl]carbamate NN157

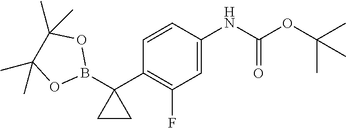 [SMILES: CC(C)(C)OC(=O)Nc1ccc(C2(B3OC(C)(C)C(C)(C)O3)CC2)c(F)c1]

To a solution of tert-butyl N-(4-bromo-3-fluoro-phenyl)carbamate NN156 (155.0 mg, 0.534 mmol), 2,2′-Cyclopropylidenebis(4,4,5,5-tetramethyl-1,3,2-dioxaborolane) (80.0 mg, 0.2667 mmol) and cesium carbonate (261.0 mg, 0.800 mmol) in 1,4-dioxane (6.3 mL) and water (0.63 mL) were added butyldi-1-adamantylphosphine (20.0 mg, 0.053 mmol) followed by palladium(II) acetate (6.0 mg, 0.027 mmol). The flask was evacuated and backfilled with N₂ (3 times) before heating at 100° C. for 16 h. Extra palladium(II) acetate (6.0 mg, 0.027 mmol), butyldi-1-adamantylphosphine (20.0 mg, 0.053 mmol) and cesium carbonate (261.0 mg, 0.800 mmol) were added under N₂ and the reaction mixture was stirred at 100° C. for 7 h. The reaction mixture was diluted with DCM (75 mL) and water (75 mL). The two layers were separated and the aqueous layer was extracted with DCM (2×75 mL). The combined organics were dried over MgSO₄, filtered and concentrated to dryness. The residue was purified by flash chromatography on silica gel (using a gradient of 0 to 20% EtOAc:Heptane as eluent) to afford the title compound as a white-orange solid (89.4 g, yield: 44%). LC-MS m/z [M+H-tBu]⁺: 322.1; purity: 100%. ¹H NMR (400 MHz, DMSO-d₆) δ 9.41 (s, 1H), 7.23 (d, J=13.0 Hz, 1H), 7.07-6.97 (m, 2H), 1.46 (s, 9H), 1.13 (s, 12H), 0.99-0.92 (m, 2H), 0.81-0.73 (m, 2H). ¹⁹F NMR (376 MHz, DMSO-d₆) δ−114.56 (dd, J=13.0, 7.5 Hz).

### Step 3: Synthesis of tert-butyl N-[3-fluoro-4-(1-fluorocyclopropyl)phenyl]carbamate NN158

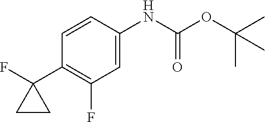 [SMILES: CC(C)(C)OC(=O)Nc1ccc(C2(F)CC2)c(F)c1]

A vial was charged with tert-butyl N-[3-fluoro-4-[1-(4,4,5,5-tetramethyl-1,3,2-dioxaborolan-2-yl)cyclopropyl]phenyl]carbamate NN157 (70.4 mg, 0.187 mmol), silver nitrate (6.0 mg, 0.035 mmol) and 1-chloromethyl-4-fluoro-1,4-diazoniabicyclo[2.2.2]octane bis(tetrafluoroborate) (202.0 mg, 0.559 mmol) and was evacuated and filled with N₂ (3 times). DCM (1 mL) was added followed by water (1 mL) and TFA (56 μL, 0.746 mmol). The resulting mixture was stirred at 50° C. for 6 h. The reaction mixture was diluted with DCM (30 mL) and water was added (30 mL). The two layers were separated and the aqueous layer was extracted with DCM (2×30 mL). The combined organics were dried over MgSO₄, filtered and concentrated to dryness. The residue was purified by flash chromatography on silica gel (using a gradient of 0 to 100% DCM:Heptane as eluent) to afford the title compound as a white solid (21.8 mg, yield: 40%). LC-MS m/z [M+H-tBu]⁺: 214.1; purity: 92%. ¹H NMR (400 MHz, DMSO-d₆) δ 9.71 (s, 1H), 7.44-7.36 (m, 2H), 7.23 (d, J=8.6 Hz, 1H), 1.40-1.28 (m, 2H), 1.06-0.97 (m, 11H). ¹⁹F NMR (376 MHz, DMSO-d₆) δ−74.67 (s), −114.84 (ddd, J=13.2, 8.6, 4.2 Hz), −163.92-−164.11 (m).

### Step 4: Synthesis of 3-fluoro-4-(1-fluorocyclopropyl)aniline hydrochloride NN159

To a solution of tert-butyl N-[3-fluoro-4-(1-fluorocyclopropyl)phenyl]carbamate NN158 (84.0 mg, 0.311 mmol) in dry DCM (3.1 mL) was added, hydrochloric acid (4N in 1,4-Dioxane, 1.6 mL, 1.4 mmol) and the reaction mixture was stirred at room temperature for 6 h. The reaction mixture was concentrated to dryness. The residue was triturated with Et₂O (5 mL). The solid was filtered and washed with cold Et₂O (4×3 mL). The solid residue was dried under high vacuum to afford the title compound as a white solid (59.4 mg, yield: 93%). Not stable in LC-MS. ¹H NMR (400 MHz, DMSO-d₆) δ 7.66 (bs., 3H), 7.36 (td, J=8.4, 1.8 Hz, 1H), 6.82-6.70 (m, 2H), 1.39-1.26 (m, 2H), 1.05-0.95 (m, 2H).

### Intermediate NN244: 2,2-difluoro-2,3-dihydro-1H-inden-5-amine

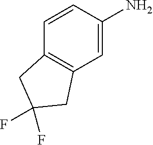 [SMILES: Nc1ccc2c(c1)CC(F)(F)C2]

### Step 1: Synthesis of 2,2-difluoro-5-nitro-2,3-dihydro-1H-indene NN245

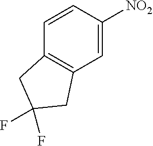 [SMILES: O=[N+]([O-])c1ccc2c(c1)CC(F)(F)C2]

To a solution of 5-nitroindan-2-one (2.00 g, 11.3 mmol) in DCM (50 mL) was added DAST (6.07 mL, 45.2 mmol) at 0° C. and the reaction mixture was stirred at room temperature for 16h. Progress of the reaction was monitored by TLC and LCMS. After completion, the reaction mixture was quenched with NaHCO₃ (150 mL) and extracted with EtOAc (2×150 mL). The organic layer was separated, dried over anhydrous Na₂SO₄ and concentrated under vacuo. The crude obtained was purified by combi-flash chromatography (10% EtOAc in hexanes) to afford 2,2-difluoro-5-nitro-2,3-dihydro-1H-indene (0.662 g, 10%) as a viscous oil. ¹H NMR (400 MHz, DMSO-d₆) δ 3.53-3.67 (m, 4H) 7.51-7.59 (m, 1H) 8.10-8.16 (m, 1H) 8.18 (s, 1H).

### Step 2: Synthesis of 2,2-difluoro-2,3-dihydro-1H-inden-5-amine NN244

To a solution of 2,2-difluoro-5-nitro-2,3-dihydro-1H-indene NN245 (0.35 g, 1.76 mmol) in EtOAc (10 mL) was added Pd/C (0.16 g, 3.01 mmol) at room temperature under argon atmosphere and the reaction mixture was stirred at room temperature for 16 h under hydrogen pressure. Progress of the reaction was monitored by TLC. After completion, the reaction mixture was filtered through the pad of Celite®, washed with EtOAc (50 mL) and the filtrate was concentrated under vacuo. The crude obtained was purified by column chromatography (silica, 100-200 mesh, 5% MeOH in DCM) to afford 2,2-difluoro-2,3-dihydro-1H-inden-5-amine (0.21 g, 71%) as an off-white solid. LC-MS (ESI) m/e [M+H]⁺: 170.0. ¹H NMR (400 MHz, DMSO-d₆) δ 3.16-3.31 (m, 4H) 5.00 (brs, 2H) 6.40-6.48 (m, 2H) 6.88 (d, J=8.31 Hz, 1H).

### Intermediate NN178: 2-(3,3-difluoroazetidin-1-yl)-5-(4,4,5,5-tetramethyl-1,3,2-dioxaborolan-2-yl)pyridin-4-amine

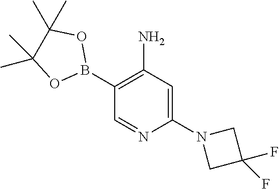 [SMILES: CC1(C)OB(c2cnc(N3CC(F)(F)C3)cc2N)OC1(C)C]

### Step 1: Synthesis of 2-(3,3-difluoroazetidin-1-yl)-4-nitro-pyridine 1-oxide NN176

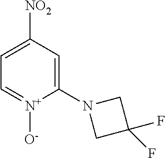 [SMILES: O=[N+]([O-])c1cc[n+]([O-])c(N2CC(F)(F)C2)c1]

To a solution of 2-Chloro-4-nitropyridine-N-oxyde (1.54 g, 8.82 mmol) and 3,3-difluoroazetidine hydrochloride (2.29 g, 17.7 mmol) in THF (7.5 mL) and EtOH (7.5 mL) was added DIPEA (3.8 mL, 22 mmol) at room temperature. The reaction mixture was stirred and heated at 90° C. for 18 h. After cooling to room temperature, the reaction mixture was diluted with water and extracted with EtOAc (3×). The combined organic extracts were washed with brine, dried over Na₂SO₄, filtered and concentrated to dryness. The crude was purified by column chromatography on silica gel (using a gradient of 0% to 10% MeOH in DCM) to afford the title compound as brown solid (1,036 g, yield: 51%). LC-MS m/z [M+H]⁺: 232.0; rt: 0.80 min; purity: 87%.

### Step 2: Synthesis of 2-(3,3-difluoroazetidin-1-yl)pyridin-4-amine NN177

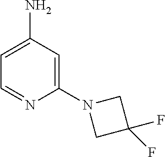 [SMILES: Nc1ccnc(N2CC(F)(F)C2)c1]

To a suspension of 2-(3,3-difluoroazetidin-1-yl)-4-nitro-pyridine 1-oxide NN176 (500 mg, 1.88 mmol) in EtOH (10 mL, 171.32 mmol) was added Pt/C (5%, 100 mg, 0,026 mmol). The reaction mixture was stirred and heated at 60° C. under 7 bars of hydrogen for 16 h. To complete the reaction, another addition of Pt/C (100 mg, 0,026 mmol) was added and stirred at 60° C. under 7 bars of hydrogen for 18 h. The reaction mixture was filtered through a pad of Celite® and washed with EtOAc (3×). The filtrate was concentrated under vacuum. The residue was purified by flash chromatography on silica gel (using a gradient of 20% to 100% AcOEt in Heptane) to afford the title compound as brown solid (243 mg, yield: 68%). LC-MS m/z [M+H]⁺: 186; purity: 99%.

### Step 3: Synthesis of 2-(3,3-difluoroazetidin-1-yl)-5-(4,4,5,5-tetramethyl-1,3,2-dioxaborolan-2-yl)pyridin-4-amine NN178

In a nitrogen filled glovebox, 4,4′-Di-tert-butyl-2,2′-dipyridyl (18 mg, 0,066 mmol) and (1,5-cyclooctadiene)(methoxy)Iridium(I) dimer (22 mg, 0,033 mmol) were weighed. Outside the box and under a stream of argon were added NN177 2-(3,3-difluoroazetidin-1-yl)pyridin-4-amine (200 mg, 1,0801 mmol), Bis(pinacolato)diboron (549 mg, 2,162 mmol) and dry THF (2.2 mL) previously degassed under Argon. The reaction mixture was stirred and heated at 80° C. for 18 h and then concentrated under vacuum to afford the title compound which was taken crude to the next step. LC-MS m/z [M+H]⁺: 312.1; purity: 77%.

### Intermediate NN151: 3-bromo-2-chloro-6-(3-fluoro-1-methyl-azetidin-3-yl)pyridin-4-amine

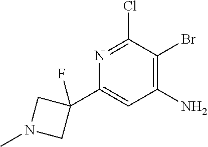

### Step 1: Synthesis of tert-butyl N-tert-butoxycarbonyl-N-(2-chloro-4-pyridyl)carbamate NN143

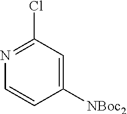

To a solution of 2-chloropyridin-4-amine (199.0 g, 1.548 mol) and DMAP (3.78 g, 31.0 mmol) in dry THE (3.1 L) was added di-tert-butyl dicarbonate (1.01 g, 4.644 mol). The resulting mixture was stirred, at room temperature, for 2 days and then, concentrated to dryness. The residue was purified by flash chromatography on silica gel (using a gradient of 0 to 30% EtOAc:Heptane as eluent). The first product fractions were triturated from diethyl ether and then combined with the latter eluting product fractions to afford the title compound as a white solid (436 g, 86%, containing some tert-butyl (2-chloropyridin-4-yl)carbamate). LC-MS m/z [M+H]⁺: 329; purity: 90%. ¹H NMR (400 MHz, CDCl₃) δ 8.38 (d, J=5.4 Hz, 1H), 7.19 (d, J=1.9 Hz, 1H), 7.05 (dd, J=5.4, 1.9 Hz, 1H), 1.47 (s, 18H).

### Step 2: Synthesis of tert-butyl N-(2-chloro-1-oxido-pyridin-1-ium-4-yl)carbamate NN144

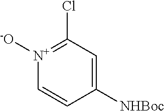

To a solution of tert-butyl N-tert-butoxycarbonyl-N-(2-chloro-4-pyridyl)carbamate NN143 (62.0 g, 189 mmol) in DCM (943.0 mL) at room temperature under N2, was added urea hydrogen peroxide (17.74 g, 189 mmol). The reaction mixture was cooled in a water bath containing some ice, and trifluoroacetic anhydride (36.7 mL, 264 mmol) was added over a 15 min period. The internal temperature rose from 16 to 19° C. and, when necessary, small portions of ice were added to the water bath to keep the reaction temperature around room temperature. After 6 h, TFA (21.79 mL, 283 mmol) was slowly added and the reaction mixture was stirred at room temperature for 2 days. The reaction mixture was diluted with a saturated aqueous solution of NaHCO₃ (400 mL) and stirred vigorously until most of the gas evolution ceased. The layers were separated, and the aqueous phase was extracted with DCM. When necessary, small portions of MeOH were added to prevent the product from precipitating from the mixture. The combined organics were washed with a saturated aqueous solution of NaHCO₃ and again small portions of MeOH were added to prevent the product from precipitating from the mixture. The organic layer was dried over Na₂SO₄ and concentrated to dryness. The residue was suspended in Et₂O (400 mL) and stirred for 30 min prior to filtration. The solids were washed with some Et₂O and dried on a gentle stream of N₂ to afford the title compound as a white solid (29.7 g, 64%). LC-MS m/z [M+H]⁺: 245; purity: 98%. ¹H NMR (400 MHz, CDCl₃) δ 8.54 (s, 1H), 8.22 (d, J=7.3 Hz, 1H), 7.93 (d, J=3.0 Hz, 1H), 7.41 (dd, J=7.3, 3.0 Hz, 1H), 1.49 (s, 9H).

### Step 3: Synthesis of tert-butyl 3-[4-(tert-butoxycarbonylamino)-6-chloro-1-oxido-pyridin-1-ium-2-yl]-3-hydroxy-azetidine-1-carboxylate NN145

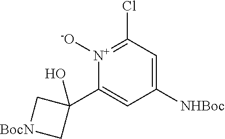

To a solution of tert-butyl N-(2-chloro-1-oxido-pyridin-1-ium-4-yl)carbamate NN144 (2.5 g, 10.0 mmol) in dry THF (60.0 mL, 739.870 mmol) at −78° C. was added dropwise (over 20 min) iPrMgCl·LiCl (1.04 M in THF, 24.1 mL, 25.1 mmol). The resulting mixture was stirred at −78° C. for 1 h. Then, a solution of 1-boc-3-azetidinone (3.0 g, 16.999 mmol) in THF (17.0 mL) was added dropwise at −78° C. and the resulting mixture was stirred at −78° C. for 1.5 h. The reaction mixture was warmed to 0° C. Under strong stirring, a saturated aqueous solution of NaHCO₃ (50 mL) was slowly added (slight gaz evolution) followed by water (300 mL), a saturated solution of rochelle's salts (100 mL) and EtOAc (100 mL). After additional vigorous stirring for 5 min at 0° C., the reaction mixture was warmed to room temperature and stirred for 16 h. The two layers were separated and the aqueous layer was extracted with a mixture of iPrOH/CHCl₃ 1/4 (4×100 mL). The combined organics were dried over MgSO₄, filtered and concentrated to dryness. The residue was purified by flash chromatography on silica gel (using a gradient of 0 to 3.5% MeOH:DCM as eluent) to afford the title compound as a yellow solid (3.33 g, yield: 54%, 68% purity). LC-MS m/z [M+H]⁺: 416.2/418.2; purity: 98%. ¹H NMR (400 MHz, DMSO-d₆) δ 10.08 (s, 1H), 7.78 (d, J=3.0 Hz, 1H), 7.71 (d, J=3.0 Hz, 1H), 6.77 (s, 1H), 4.44 (m, 2H), 3.78 (m, 2H), 1.48 (s, 9H), 1.39 (s, 9H).

### Step 4: Synthesis of tert-butyl 3-[4-(tert-butoxycarbonylamino)-6-chloro-1-oxido-pyridin-1-ium-2-yl]-3-fluoro-azetidine-1-carboxylate NN146

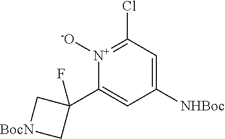

To a stirred solution of tert-butyl 3-[4-(tert-butoxycarbonylamino)-6-chloro-1-oxido-pyridin-1-ium-2-yl]-3-hydroxy-azetidine-1-carboxylate NN145 (3.33 g, 5.44 mmol) in DCM (55.0 mL) at 0° C. under N₂ was added diethylaminosulfur trifluoride (1.50 mL, 11 mmol) and the resulting solution was stirred at 0° C. for 1.5 h. At 0° C., a saturated aqueous solution of NaHCO₃ (100 mL) was slowly added (potential gas evolution) and the aqueous layer was extracted with DCM (3×50 mL). The combined organics were dried over MgSO₄, filtered and concentrated to dryness. The residue was purified by flash chromatography on silica gel (using a gradient of 10 to 60% EtOAc:Heptane as eluent) to afford the title compound as a yellow solid (1.43 g, yield: 52%). LC-MS m/z [M+H]⁺: 418.2/420.1; purity: 98%. ¹H NMR (400 MHz, DMSO-d₆) δ 10.14 (s, 1H), 7.84 (d, J=3.0 Hz, 1H), 7.65 (d, J=3.0 Hz, 1H), 4.67 (m, 2H), 4.06 (m, 2H), 1.49 (s, 9H), 1.40 (s, 9H). 19F NMR (376 MHz, DMSO-d₆) δ−149.52 (p, J=24.4 Hz). Selective HOESY NMR (400 MHz, DMSO-d₆) δ 7.65 (s), 4.14-4.03 (m).

### Step 5: Synthesis of tert-butyl 3-[4-(tert-butoxycarbonylamino)-6-chloro-2-pyridyl]-3-fluoro-azetidine-1-carboxylate NN147

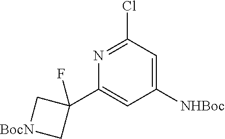 [SMILES: CC(C)(C)OC(=O)Nc1cc(Cl)nc(C2(F)CN(C(=O)OC(C)(C)C)C2)c1]

To a mixture of activated zinc (775.0 mg, 11.9 mmol) and sodium iodide (2.67 g, 17.8 mmol) in THF (5.9 mL) at 0° C., was added by a slow dropwise over 10 min (exothermic reaction), a solution of tert-butyl 3-[4-(tert-butoxycarbonylamino)-6-chloro-1-oxido-pyridin-1-ium-2-yl]-3-fluoro-azetidine-1-carboxylate NN146 (2.88 g, 5.93 mmol) and chlorotrimethylsilane (2.30 mL, 18 mmol) in THF (4.6 mL) (the vial was washed with additional THF (2.0 mL)). The reaction mixture was stirred at 0° C. for 15 min. The reaction mixture was directly filtered over a Celite pad (EtOAc was used as washing solvent). The filtrate was concentrated under vacuum and purified by flash chromatography on silica gel (using a gradient of 0 to 30% EtOAc:Heptane as eluent) to afford the title compound (2.10 g, yield: 88%) as a white solid. LC-MS m/z [M+H-tBu]⁺: 346.1/348.1; purity: 100%. ¹H NMR (400 MHz, DMSO-d₆) δ 10.28 (s, 1H), 7.60 (s, 1H), 7.52 (s, 1H), 4.40-4.11 (m, 4H), 1.49 (s, 9H), 1.42 (s, 9H). ¹⁹F NMR (376 MHz, DMSO-d₆) δ−157.33 (p, J=21.2 Hz). Selective HOESY NMR (400 MHz, DMSO-d₆) δ 7.60 (s), 4.27-4.13 (m).

### Step 6: Synthesis of tert-butyl N-[2-chloro-6-(3-fluoroazetidin-3-yl)-4-pyridyl]carbamate NN148

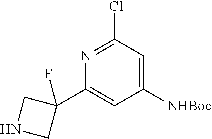 [SMILES: CC(C)(C)OC(=O)Nc1cc(Cl)nc(C2(F)CNC2)c1]

To a solution of tert-butyl 3-[4-(tert-butoxycarbonylamino)-6-chloro-2-pyridyl]-3-fluoro-azetidine-1-carboxylate NN147 (2.04 g, 5.08 mmol) in dry DCM (51.0 mL), at 0° C., was added TEA (1.10 mL, 7.80 mmol) followed by trimethylsilyl trifluoromethanesulfonate (1.40 mL, 7.60 mmol). The reaction mixture was stirred at 0° C. for 1 h. Water (150 mL) was added and the aqueous layer was extracted with DCM (3×40 mL) and the combined organics were concentrated to dryness to afford the title compound as a white solid (1.67 g, yield: quantitative). LC-MS m/z [M+H]⁺: 302.1/304.1; purity: 95%. ¹H NMR (400 MHz, DMSO-d₆) δ 10.23 (s, 1H), 7.57 (s, 1H), 7.50 (s, 1H), 4.02 (dd, J=22.5, 10.2 Hz, 2H), 3.79 (dd, J=22.5, 10.2 Hz, 2H), 1.48 (s, 9H). ¹⁹F NMR (376 MHz, DMSO-d₆) δ−149.64 (p, J=22.5 Hz). 1H under wayer peak. Selective HOESY NMR (400 MHz, DMSO-d₆) δ 7.59 (s), 3.81 (dd, J=22.5, 10.7 Hz)

### Step 7: Synthesis of tert-butyl N-[2-chloro-6-(3-fluoro-1-methyl-azetidin-3-yl)-4-pyridyl]carbamate NN149

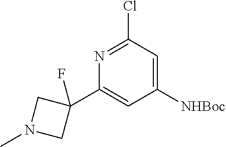 [SMILES: CN1CC(F)(c2cc(NC(=O)OC(C)(C)C)cc(Cl)n2)C1]

To a solution of tert-butyl N-[2-chloro-6-(3-fluoroazetidin-3-yl)-4-pyridyl]carbamate NN148 (1.67 g, 5.09 mmol) in dry MeOH (51.0 mL) were added AcOH (877.0 μL, 15.27 mmol) and formaldehyde (37% in water) (570.0 μL, 7.61 mmol). The reaction mixture was stirred at room temperature for 1 h. Sodium cyanoborohydride (505.0 mg, 7.63 mmol) was added and the reaction mixture was allowed to stir at room temperature for 16 h. A saturated aqueous solution of NaHCO₃ (100 mL), a saturated aqueous solution of K₂CO₃ (30 mL) and DCM (50 mL) were added and the mixture was vigorously stirred for 10 min. The aqueous layer was extracted with DCM (4×40 mL). The combined organic layers were washed with Brine (50 mL), dried over MgSO₄, filtered and concentrated to dryness to afford the title compound as a white solid (1.53 g, yield: 82%). LC-MS m/z [M+H]⁺: 316.0/317.9; purity: 96%. ¹H NMR (400 MHz, DMSO-d₆) δ 10.23 (s, 1H), 7.60 (s, 1H), 7.50 (s, 1H), 3.69-3.48 (m, 4H), 2.35 (s, 3H), 1.49 (s, 9H). ¹⁹F NMR (376 MHz, DMSO-d₆) δ−152.19 (p, J=21.2 Hz). Selective HOESY NMR (400 MHz, DMSO-d₆) δ 7.60 (s), 3.67-3.50 (m).

### Step 8: Synthesis of 2-chloro-6-(3-fluoro-1-methyl-azetidin-3-yl)pyridin-4-amine NN150

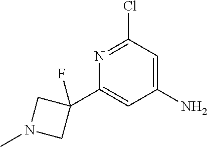

To a solution of tert-butyl N-[2-chloro-6-(3-fluoro-1-methyl-azetidin-3-yl)-4-pyridyl]carbamate NN149 (1.53 g, 4.17 mmol) in DCM (8.3 mL), at 0° C., was added TFA (8.3 mL) and the resulting mixture was stirred at room temperature for 30 min. The reaction mixture was concentrated to dryness to afford the title compound which was used as such in the next step. LC-MS m/z [M+H]⁺: 216.1/218.2; purity: 89%. ¹H NMR (400 MHz, DMSO-d₆) δ 6.62 (d, J=1.8 Hz, 1H), 6.56 (s, 2H), 6.45 (d, J=1.8 Hz, 1H), 3.66-3.44 (m, 4H), 2.33 (s, 3H). ¹⁹F NMR (376 MHz, DMSO-d₆) δ−152.78 (p, J=22.5 Hz).

### Step 9: Synthesis of 3-bromo-2-chloro-6-(3-fluoro-1-methyl-azetidin-3-yl)pyridin-4-amine

To a solution of 2-chloro-6-(3-fluoro-1-methyl-azetidin-3-yl)pyridin-4-amine NN150 (3.25 g, 4.17 mmol) in MeCN (10.0 mL) was added dropwise, at room temperature, a solution of NBS (805.0 mg, 4.52 mmol) in MeCN (2.5 mL). The resulting mixture was stirred at room temperature for 16 h. Water (10 mL) and EtOAc (5 mL) were added and the aqueous layer was extracted with EtOAc (3×5 mL). The combined organics were dried over MgSO₄, filtered and concentrated to dryness. The residue was purified by preparative HPLC (Method G_B) to afford the title compound as a beigeish solid (810.8 mg, yield: 65%). LC-MS m/z [M+H]⁺: 294/296/298; purity: 98%. ¹H NMR (400 MHz, DMSO-d₆) δ 6.84 (s, 2H), 6.79 (s, 1H), 3.66-3.44 (m, 4H), 2.33 (s 3H). ¹⁹F NMR (376 MHz, DMSO-d₆) δ−152.00 (p, J=21.6 Hz). Selective HOESY NMR (400 MHz, DMSO-d₆) δ 6.79 (s), 3.65-3.43 (m).

### Intermediate NN165: 3-bromo-2-chloro-6-cyclopropyl-pyridin-4-amine

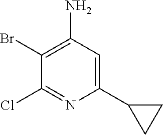 [SMILES: Nc1cc(C2CC2)nc(Cl)c1Br]

### Step 1: Synthesis of tert-butyl N-tert-butoxycarbonyl-N-(2,6-dichloro-4-pyridyl)carbamate NN163

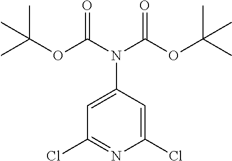 [SMILES: CC(C)(C)OC(=O)N(C(=O)OC(C)(C)C)c1cc(Cl)nc(Cl)c1]

To a solution of 4-amino-2,6-dichloropyridine (5.00 g, 30.1 mmol) in THF (300 mL) were added DMAP (371 mg, 3.01 mmol) and Boc₂O (20.0 g, 89.8 mmol). The reaction mixture was stirred at room temperature for 18 h. After complete conversion, the reaction mixture was concentrated to dryness. The residue was dissolved in DCM (150 mL), washed with water (2×150 mL) and with brine (150 mL). The organic extract was dried over MgSO₄, filtered and concentrated under vacuum. The residue was purified by flash chromatography on silica gel (using a gradient of 0 to 20% EtOAc in Heptane as eluent) to afford the title compound (10.1 g, yield: 84%) as a white solid.

LC-MS m/z: [M+H]⁺: 363.0/365.1; purity: 91%. ¹H NMR (400 MHz, DMSO-d₆) δ 7.79-7.61 (m, 2H), 1.42 (s, 18H).

### Step 2: Synthesis of: 2-chloro-6-cyclopropyl-pyridin-4-amine NN164

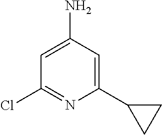 [SMILES: Nc1cc(Cl)nc(C2CC2)c1]

Under N₂ atmosphere, to a suspension of tert-butyl N-tert-butoxycarbonyl-N-(2,6-dichloro-4-pyridyl)carbamate (Intermediate NN163, 2.00 g, 5.01 mmol), potassium cyclopropyltrifluoroborate (772 mg, 5.06 mmol) and Cs₂CO₃ (4.90 g, 15.0 mmol) in Toluene (17.5 mL) and water (1.75 mL) were added Pd(OAc)₂ (22.5 mg, 0.10 mmol) and CataCXium A (56.7 mg, 0.15 mmol). The reaction mixture was sonicated a few seconds and stirred at 100° C. for 4 h. After complete conversion, water (100 mL) was added and the reaction mixture was extracted with EtOAc (3×100 mL). The combined organic extracts were dried over MgSO₄, filtered and concentrated under vacuum. The residue was dissolved in DCM (35 mL) before addition of TFA (35 mL). The reaction mixture was stirred at room temperature for 2 h. After complete conversion, the reaction mixture was concentrated under vacuum. The residue was dissolved in DCM (100 mL) and quenched with a 10% w/w NH₄OH solution in MeOH (20 mL) and with water (100 mL). The aqueous phase was extracted with DCM (2×100 mL). The combined organic extracts were dried over MgSO₄, filtered and concentrated under vacuum. The residue was purified by preparative HPLC (Method G_A) then by reverse phase flash chromatography. The purified compound was dissolved in DCM (100 mL) and washed with a 10% w/w NH₄OH solution in MeOH (20 mL) and with water (100 mL). The aqueous phase was extracted with DCM (2×100 mL). The combined organic extracts were dried over MgSO₄, filtered and concentrated under vacuum to afford the title compound (465 mg, yield: 48%) as a brownish solid. LC-MS m/z: [M+H]⁺: 169.1/171.1; purity: 94%. ¹H NMR (400 MHz, DMSO-d₆) δ 6.37-6.32 (m, 1H), 6.29-6.18 (m, 3H), 1.87-1.76 (m, 1H), 0.86-0.74 (m, 4H).

### Step 3: Synthesis of: 3-bromo-2-chloro-6-cyclopropyl-pyridin-4-amine NN165

To a solution of 2-chloro-6-cyclopropyl-pyridin-4-amine (Intermediate NN164, 460 mg, 2.37 mmol) in MeCN (12 mL) at 0° C., was added NBS (444 mg, 2.49 mmol). The reaction mixture was stirred at room temperature for 2 h. Extra NBS (106 mg, 0.60 mmol) was added and the reaction mixture was stirred at room temperature for 18 h. After complete conversion, water (80 mL) was added and the reaction mixture was extracted with DCM (3×80 mL). The combined organic extracts were dried over MgSO₄, filtered, and concentrated under vacuum. The residue was purified by flash chromatography on silica gel (using a gradient of 0 to 20% EtOAc in Heptane as eluent) to afford the title compound (342 mg, yield: 58%) as a white solid. LC-MS m/z: [M+H]⁺: 247.0/249.0/251.0; purity: 100%. ¹H NMR (400 MHz, DMSO-d₆) δ 6.52 (s, 1H), 6.50 (s, 2H), 1.84 (tt, J=8.6, 4.8 Hz, 1H), 0.89-0.81 (m, 2H), 0.80-0.72 (m, 2H).

### Intermediate NN175: 3-bromo-2-chloro-6-(difluoromethoxy)pyridin-4-amine

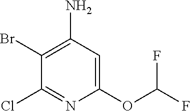 [SMILES: Nc1cc(OC(F)F)nc(Cl)c1Br]

### Step 1: Synthesis of 4-amino-6-chloro-pyridin-2-ol NN172

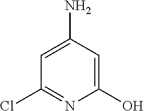

To a suspension of 2-chloro-6-methoxypyridin-4-amine (1.00 g, 6.31 mmol) and KI (10.0 g, 60.2 mmol) in MeCN (32 mL) was added TMSCl (8 mL, 62.4 mmol). The reaction mixture was stirred at 80° C. for 20 h. Extra TMSCl (2 mL, 15.6 mmol) was added and the reaction mixture was stirred at 80° C. for 3 h. The reaction mixture was concentrated under vacuum. The residue was purified by reverse phase chromatography (Method G_B, gradient from 5% to 30% ACN) then by a second reverse phase chromatography (Method G_B, gradient from 5% to 20% ACN) to afford the title compound (400 mg, yield: 37%) as a white solid. LC-MS m/z: [M+H]⁺: 144.9/146.9; purity: 83%. ¹H NMR (400 MHz, DMSO-d₆) δ 6.14 (s, 2H), 6.02 (d, J=1.6 Hz, 1H), 5.58 (d, J=1.6 Hz, 1H). OH proton not observed.

### Step 2: Synthesis of: 2-chloro-6-(difluoromethoxy)pyridin-4-amine NN173

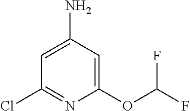 [SMILES: Nc1cc(Cl)nc(OC(F)F)c1]

To a solution of 4-amino-6-chloro-pyridin-2-ol (Intermediate NN172, 399 mg, 2.35 mmol) in MeCN (4.7 mL) was added a 6 M aqueous KOH solution (4.7 mL, 28.2 mmol). Difluoromethyl trifluoromethanesulfonate (940 μL, 7.07 mmol) was added dropwise. The reaction mixture was stirred at room temperature for 2 h. The reaction mixture was quenched by addition of saturated aqueous CsF solution (4 mL) and water (100 mL) and then extracted with DCM (3×100 mL). The combined organic extracts were dried over MgSO₄, filtered, and concentrated under vacuum to afford the title compound (403 mg, yield: 72%) as a yellow oil. LC-MS m/z: [M+H]⁺: 195.1/197.1; purity: 74%. ¹H NMR (400 MHz, DMSO-d₆) δ 7.49 (t, J=73.1 Hz, 1H), 6.71 (s, 2H), 6.41 (d, J=1.6 Hz, 1H), 6.00 (d, J=1.6 Hz, 1H). ¹⁹F NMR (376 MHz, DMSO-d₆) δ−86.61 (d, J=73.1 Hz).

### Step 3: Synthesis of 3-bromo-2-chloro-6-(difluoromethoxy)-5-iodo-pyridin-4-amine NN174

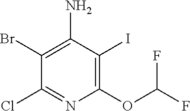 [SMILES: Nc1c(Br)c(Cl)nc(OC(F)F)c1I]

To a solution of 2-chloro-6-(difluoromethoxy)pyridin-4-amine (Intermediate NN173, 332 mg, 1.39 mmol) in MeCN (7 mL) at 0° C., was added dropwise a solution of NIS (492 mg, 2.08 mmol) in MeCN (3.5 mL). The reaction mixture was stirred at room temperature for 2 h then at 80° C. for 17 h. After complete conversion, the reaction mixture was concentrated under vacuum. Water (7.0 mL) and MeCN (1.7 mL) were added to the residue followed by HBr (47% w/w in water, 360 μL, 2.83 mmol) and H₂O₂ (35% w/w in water, 480 μL, 5.61 mmol). The reaction mixture was stirred at 40° C. for 20 h. The reaction mixture was concentrated under vacuum. The residue was purified by flash chromatography on silica gel (using a gradient of 0 to 10% EtOAc in Heptane as eluent) to afford the title compound (542 mg, yield: 40%) as a white solid. LC-MS m/z: [M+H]⁺: 398.8/400.8; purity: 41%.

### Step 4: Synthesis of 3-bromo-2-chloro-6-(difluoromethoxy)pyridin-4-amine NN175

Under N₂ atmosphere, to a solution of 3-bromo-2-chloro-6-(difluoromethoxy)-5-iodo-pyridin-4-amine (Intermediate NN174, 564 mg, 0.59 mmol) in dry THF (3.5 mL) at −40° C. was added iPrMgCl—LiCl (1.30 M in THF, 0.45 mL, 0.59 mmol). The reaction mixture was stirred at −40° C. for 30 min. Extra iPrMgCl—LiCl (1.30 M in THF, 0.45 mL, 0.59 mmol) was added and the reaction mixture was stirred at −40° C. for 30 min. Extra iPrMgCl—LiCl (1.30 M in THF, 0.67 mL, 0.87 mmol) was added and the reaction mixture was stirred at −40° C. for 30 min. The reaction mixture was quenched by addition of saturated aqueous NH₄Cl solution (10 mL) and water (70 mL) and extracted with DCM (3×70 mL). The combined organic extracts were dried over MgSO₄, filtered, and concentrated under vacuum. The residue was purified by flash chromatography on silica gel (using a gradient of 0 to 20% EtOAc in Heptane as eluent) to afford the title compound (146 mg, yield: 91%) as a beige solid. LC-MS m/z: [M+H]⁺: 273.0/275.0; purity: 100%. ¹H NMR (400 MHz, DMSO-d₆) δ 7.49 (t, J=72.7 Hz, 1H), 6.96 (s, 2H), 6.20 (s, 1H). Intermediate NN170: 3-bromo-2-chloro-6-methoxy-pyridin-4-amine

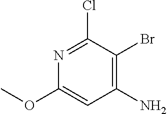

### Step 1: Synthesis of 3-bromo-2-chloro-5-iodo-6-methoxy-pyridin-4-amine NN169

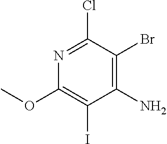

To a solution of 2-chloro-6-methoxypyridin-4-amine (400.0 mg, 2.522 mmol) in MeCN (12.6 mL) at 0° C., was added dropwise a solution of NIS (609.0 mg, 2.572 mmol) in MeCN (6.2 mL). After 6 h stirring at room temperature, NBS (500.0 mg, 2.8093 mmol) was added and the reaction mixture was stirred at room temperature for 16 h. The reaction mixture was concentrated to dryness and the residue was purified by flash chromatography on silica gel (using a gradient of 0 to 10% EtOAc:Heptane as eluent) to afford the title compound as a white solid (757 mg, yield: 55%). LC-MS (Method A2) m/z [M+H]⁺: 362.8/364.8/366.9; rt: 4.94 min; purity: 67%. LC-MS (Method B2) m/z [M+H]⁺: 362.8/364.8/366.7; rt: 4.71 min; purity: 69%. ¹H NMR (400 MHz, DMSO-d₆) δ 6.40 (s, 2H), 3.80 (s, 3H).

### Step 2: Synthesis of 3-bromo-2-chloro-6-methoxy-pyridin-4-amine NN170

Under N2, to a solution of 3-bromo-2-chloro-5-iodo-6-methoxy-pyridin-4-amine NN169 (670.7 mg, 1.255 mmol) in dry THF (7.0 mL), at −40° C., was added iPrMgCl·LiCl (0.96 M in THF, 1.3 mL, 1.2 mmol). The resulting reaction mixture was stirred at −40° C. for 30 min. Extra iPrMgCl·LiCl (0.96 M in THF, 1.3 mL, 1.2 mmol) was added and the reaction mixture was stirred at −40° C. for 30 min.

Extra iPrMgCl·LiCl (0.96 M in THF, 1.3 mL, 1.2 mmol) was added and the reaction mixture was stirred at −40° C. for 30 min. The reaction was quenched by the addition of a saturated aqueous solution of NH₄Cl (10 mL). The reaction mixture was diluted with DCM (80 mL) and water (80 mL). The aqueous layer was extracted with DCM (2×80 mL). The combined organics were dried over MgSO₄, filtered and concentrated to dryness. The residue was purified by flash chromatography on silica gel (using a gradient of 0 to 20% EtOAc:Heptane as eluent) to afford the title compound as a yellow solid (310 mg, yield: 100%). LC-MS m/z [M+H]⁺: 236.0/239.0/241.0; purity: 97%. ¹H NMR (400 MHz, DMSO-d₆) δ 6.52 (bs., 2H), 6.00 (s, 1H), 3.72 (s, 3H).

### Intermediate NN161: 3-bromo-6-(difluoromethoxy)-2-fluoro-pyridin-4-amine

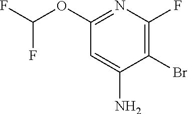 [SMILES: Nc1cc(OC(F)F)nc(F)c1Br]

### Step 1: Synthesis of 5-bromo-2-(difluoromethoxy)-6-fluoro-3-iodo-pyridin-4-amine NN160

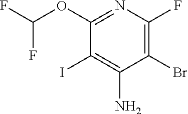 [SMILES: Nc1c(Br)c(F)nc(OC(F)F)c1I]

To a solution of 2-(difluoromethoxy)-6-fluoropyridin-4-amine (400 mg, 2.25 mmol) in MeCN (11 mL) at 0° C. was added dropwise a solution of NIS (540 mg, 2.27 mmol) in MeCN (3 mL). The reaction mixture was stirred at 80° C. for 19 h. The reaction mixture was concentrated under vacuum. Water (11.2 mL) and MeCN (2.80 mL) were added at room temperature to the residue followed by HBr (47% w/w in water, 570 μL, 4.48 mmol) and H₂O₂ (35% w/w in water, 770 μL, 8.99 mmol). The reaction mixture was stirred at 40° C. for 2 h. The reaction mixture was concentrated under vacuum and then lyophilized. The residue was purified by flash chromatography on silica gel (using a gradient of 0 to 10% EtOAc in Heptane as eluent) to afford the title compound (708 mg, yield: 68%) as a white solid. LC-MS m/z: 383.1/385.1; purity: 83%. ¹H NMR (400 MHz, DMSO-d₆) δ 7.51 (t, J=72.0 Hz, 1H), 6.91 (s, 2H). ¹⁹F NMR (376 MHz, DMSO-d₆) δ−71.18, −87.28 (d, J=72.0 Hz).

### Step 2: Synthesis of 3-bromo-6-(difluoromethoxy)-2-fluoro-pyridin-4-amine NN161

Under N₂ atmosphere, to a solution of 5-bromo-2-(difluoromethoxy)-6-fluoro-3-iodo-pyridin-4-amine (Intermediate NN160, 586 mg, 0.81 mmol) in dry THF (4.6 mL) at −40° C. was added iPrMgCl—LiCl (1.30 M in THF, 0.78 mL, 0.82 mmol). The reaction mixture was stirred at −40° C. for 30 min. Extra iPrMgCl—LiCl (1.30 M in THF, 0.78 mL, 0.82 mmol) was added and the reaction mixture was stirred at −40° C. for 30 min. Extra iPrMgCl—LiCl (1.30 M in THF, 1.16 mL, 1.22 mmol) was added and the reaction mixture was stirred at −40° C. for 30 min. The reaction mixture was quenched by addition of saturated aqueous NH₄C1 solution (10 mL) and water (70 mL). The reaction mixture was extracted with DCM (3×70 mL). The combined organic extracts were dried over MgSO₄, filtered, and concentrated under vacuum. The residue was purified by flash chromatography on silica gel (using a gradient of 0 to 10% EtOAc in Heptane as eluent) to afford the title compound (78 mg, yield: 32%) as a white solid. LC-MS m/z: [M+H]⁺: 257.0/258.9; purity: 93%. LC-MS (Method B2) m/z: [M+H]⁺: 257.0/258.9; rt: 4.07 min; purity: 92%. ¹H NMR (400 MHz, DMSO-d₆) δ 7.46 (t, J=72.7 Hz, 1H), 7.04 (s, 2H), 6.15 (s, 1H). ¹⁹F NMR (376 MHz, DMSO-d₆) δ−68.92, −86.64 (d, J=72.7 Hz).

### Intermediate NN215: 5-chloro-3-fluoro-2-(4,4,5,5-tetramethyl-1,3,2-dioxaborolan-2-yl)aniline

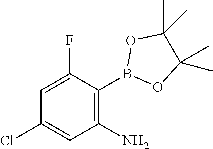 [SMILES: CC1(C)OB(c2c(N)cc(Cl)cc2F)OC1(C)C]

A flame-dried 250 mL, 3-neck, round-bottom flask under nitrogen was charged with bis(pinacolato)diboron (15 g, 58 mmol), (1,5-cyclooctadiene)(methoxy)iridium(I) dimer (190 mg, 0.28 mmol) and 4,4′-di-tert-butyl-2,2′-bipyridyl (160 mg, 0.58 mmol). The flask was evacuated and backfilled with nitrogen three times prior to the addition of a solution of 3-chloro-5-fluoroaniline (4.3 g, 29 mmol) in anhydrous tetrahydrofuran (60 mL) under nitrogen. The stirred mixture was heated at 65° C. (internal temperature) for 19 hours before it was cooled to RT. Methanol (30 mL) was added carefully to the cooled reaction mixture and the resulting solution stirred for 30 minutes at room temperature and then concentrated under vacuo. Purification by flash chromatography (Biotage Selekt, Sfar Silica HCD, 100 g, 0-20% ethyl acetate in hexane) afforded 5-chloro-3-fluoro-2-(4,4,5,5-tetramethyl-1,3,2-dioxaborolan-2-yl)aniline (6.60 g, 23 mmol, 80% Yield) an off-white solid. LC-MS m/z: 272.0 [M+H]+; purity: 96%. ¹H NMR (300 MHz, DMSO-d₆) δ 6.53-6.44 (m, 1H), 6.28 (dd, J=9.7, 1.9 Hz, 1H), 6.11 (s, 2H), 1.28 (s, 12H).

### Intermediate NN220: 2-(difluoromethyl)pyridin-4-amine NN220

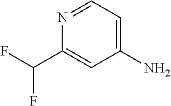

### Step 1: Synthesis of N-[2-(difluoromethyl)-4-pyridyl]-1,1-diphenyl-methanimine NN219

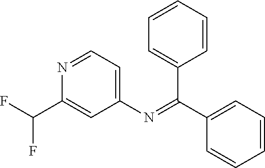 [SMILES: FC(F)c1cc(N=C(c2ccccc2)c2ccccc2)ccn1]

To a flame-dried 500 mL, 2-neck round-bottom flask attached to an air condenser were added tris(dibenzylideneacetone)dipalladium(0) (2.2 g, 2.4 mmol), tBuXPhos (2.1 g, 4.8 mmol) and sodium tert-butoxide (14 g, 140 mmol), The flask was degassed and back-filled with nitrogen three times. A solution of 4-bromo-2-(difluoromethyl)pyridine (10.1 g, 47 mmol) in anhydrous toluene (240 mL) under nitrogen was added followed by benzophenone imine (17 mL, 96 mmol). The stirred mixture was degassed and back-filled with nitrogen three times and the resulting reaction mixture was stirred at 100° C. for 2 hours before it was cooled to rt. The cooled reaction mixture was diluted with ethyl acetate (500 mL) and washed with water (400 mL). The separated organic layer was washed with brine (400 mL), dried over sodium sulfate and concentrated under vacuo. Purification by flash chromatography (Silica, gradient 0-20% ethyl acetate in iso-hexane) afforded N-[2-(difluoromethyl)-4-pyridyl]-1,1-diphenyl-methanimine (17.5 g, 45 mmol, 96% Yield) as an orange oil. LC-MS m/z: 309.2 [M+H]+; purity: 80%. ¹H NMR (300 MHz, DMSO-d₆) δ 8.36 (dd, J=5.3, 0.7 Hz, 1H), 7.85-7.06 (m, 10H), 7.01 (d, J=1.9 Hz, 1H), 6.98-6.53 (m, 2H).

### Step 2: Synthesis of Intermediate 2-(difluoromethyl)pyridin-4-amine NN220

To a stirred solution of N-[2-(difluoromethyl)-4-pyridyl]-1,1-diphenyl-methanimine NN219 (17.5 g, 45 mmol) in tetrahydrofuran (200 mL) was added concentrated HCl (37%, 20 mL, 240 mmol). The reaction mixture was stirred at rt for 1.5 hours before it was concentrated under vacuo to afford the crude product as a yellow crystallising residue. The crude was azeotropically co-evaporated with methanol (2×30 mL) prior to trituration with dichloromethane, which gave crude hydrochloride salt (8.57 g) as a pale yellow solid. The salt was free-based by chromatography (Isolute Flash SCX-2, 20 g×4) eluted with 50% methanol in dichloromethane followed by 100% methanol, followed by 7N ammonia in methanol. Fractions enriched with product were combined, evaporated down and the resulting oil azeotropically co-evaporated with ethyl acetate (2×50 mL) to give 2-(difluoromethyl)pyridin-4-amine (5.82 g, 39 mmol, 86% Yield) as a pale yellow oil. LC-MS m/z: 145.0 [M+H]+; purity: 100%. ¹H NMR (300 MHz, DMSO-d₆) δ 8.09-7.95 (m, 1H), 6.87-6.44 (m, 3H), 6.37 (s, 2H). ¹⁹F NMR (282 MHz, DMSO-d₆) δ−115.62 (d, J=55.4 Hz).

### Intermediate NN231: 2-(difluoromethyl)-5-(4,4,5,5-tetramethyl-1,3,2-dioxaborolan-2-yl)pyridin-4-amine

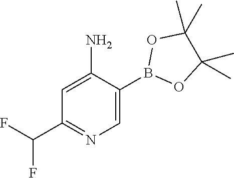 [SMILES: CC1(C)OB(c2cnc(C(F)F)cc2N)OC1(C)C]

To a flame-dried 500 mL, 2-neck round-bottom flask under nitrogen were added bis(pinacolato)diboron (20 g, 78 mmol), (1,5-cyclooctadiene)(methoxy)iridium(I) dimer (520 mg, 0.78 mmol) and 4,4′-di-tert-butyl-2,2′-dipyridyl (440 mg, 1.6 mmol). The flask was evacuated and back-filled with nitrogen 3 times prior to the addition of a solution of 2-(difluoromethyl)pyridin-4-amine NN220 (5.81 g, 39 mmol) in anhydrous 1,4-dioxane (100 mL) under nitrogen. The reaction mixture was stirred at 80° C. for 20 hours before it was cooled down to rt. The solution mixture was used as such in the next step with cautious care (gas evolution in the next step).

### Intermediate NN232: 2-methoxy-5-(4,4,5,5-tetramethyl-1,3,2-dioxaborolan-2-yl)pyridin-4-amine

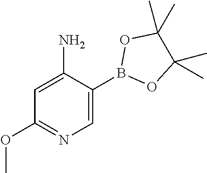 [SMILES: COc1cc(N)c(B2OC(C)(C)C(C)(C)O2)cn1]

To a flame-dried 100 mL, 3-neck round-bottom flask under nitrogen were added 4-amino-2-methoxypyridine (1.0 g, 7.8 mmol), bis(pinacolato)diboron (4.0 g, 16 mmol), (1,5-cyclooctadiene)(methoxy)iridium(I) dimer (100 mg, 0.15 mmol) and 4,4′-di-tert-butyl-2,2′-dipyridyl (90 mg, 0.32 mmol). The flask was evacuated and back-filled with nitrogen three times prior to the addition of anhydrous 1,4-dioxane (20 mL). The reaction mixture was stirred at 75° C. (internal temperature) for 17 hours before it was cooled down to RT. The solution mixture was used as such in the next step with cautious care (gas evolution in the next step).

### Intermediate NN233: 3-amino-5-fluoro-4-(4,4,5,5-tetramethyl-1,3,2-dioxaborolan-2-yl)benzonitrile

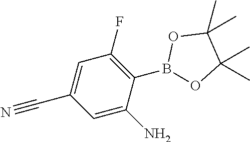 [SMILES: CC1(C)OB(c2c(N)cc(C#N)cc2F)OC1(C)C]

Under nitrogen, to a solution of 3-amino-5-fluoro-benzonitrile (1.0 g, 7.4 mmol) in THF (15 mL) was added 4,4,5,5-tetramethyl-2-(4,4,5,5-tetramethyl-1,3,2-dioxaborolan-2-yl)-1,3,2-dioxaborolane (2.8 g, 0.011 mol), (1,5-cyclooctadiene)(methoxy)iridium(I) dimer (0.097 g, 0.15 mmol) and 4,4′-di-tert-butyl-2,2′-bipyridine (79 mg, 0.30 mmol). The reaction mixture was heated at reflux overnight then cooled to RT. The reaction mixture was filtered through a pad of celite and washed with EtOAc (100 mL) then concentrated under reduced pressure. The crude was purified by column chromatography on silica gel (using a gradient of 0-60% EtOAc in isohexane as eluent) to afford the title compound as a white solid (1.0 g, yield: 51%). ¹H NMR (400 MHz, DMSO-d₆) δ 6.80 (d, J=1.4 Hz, 1H), 6.64 (dd, J=9.4, 1.3 Hz, 1H), 6.25 (s, 2H), 1.30 (s, 12H); purity: 95%.

### Intermediate NN225: 5-bromo-2-(4,4,5,5-tetramethyl-1,3,2-dioxaborolan-2-yl)aniline

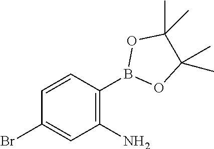 [SMILES: CC1(C)OB(c2ccc(Br)cc2N)OC1(C)C]

To a flame-dried 500 mL, 3-neck round-bottom flask under nitrogen were added bis(pinacolato)diboron (61 g, 240 mmol), (1,5-cyclooctadiene)(methoxy)iridium(I) dimer (820 mg, 1.2 mmol) and 3,4,7,8-tetramethyl-1,10-phenanthroline (580 mg, 2.4 mmol). The flask was evacuated and backfilled with nitrogen three times prior to the addition of a solution of 3-bromoaniline (21 g, 120 mmol) in anhydrous tetrahydrofuran (240 mL) under nitrogen. The stirred mixture was heated at 60° C. (internal temperature) for 15 hours before it was cooled down to rt. To the cooled reaction mixture was added dropwise methanol (100 mL) with extreme care. The resulting solution was stirred for 30 minutes at room temperature and then concentrated under vacuo to afford crude product as a dark residue. Purification by flash chromatography (Silica, gradient 0-10% ethyl acetate in iso-hexane) with a gradient of 0% to 10% ethyl acetate in iso-hexane) afforded 5-bromo-2-(4,4,5,5-tetramethyl-1,3,2-dioxaborolan-2-yl)aniline (11.6 g, 37 mmol, 31% Yield) as a pale tan oil which crystallised out on standing. LC-MS m/z: 298.2/300.2 [M+H]+; purity: 100%. ¹H NMR (300 MHz, CDCl₃) δ 7.44 (d, J=7.9 Hz, 1H), 6.82-6.73 (m, 2H), 4.75 (s, 2H), 1.33 (s, 12H).

### Intermediate NN246: 5-bromo-2-(difluoromethyl)pyridin-4-amine

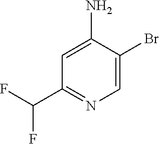 [SMILES: Nc1cc(C(F)F)ncc1Br]

### Synthesis of 5-bromo-2-(difluoromethyl)pyridin-4-amine

NBS (308 mg, 1.73 mmol) was added to a solution of 2-(difluoromethyl)pyridin-4-amine NN220 (250 mg, 1.65 mmol) in DCM (10.0 mL) at 0° C. The reaction mixture was stirred at RT overnight and water (30 mL) was added. The phases were separated and the aqueous layer was extracted with DCM (2×30 mL). The combined extracts were dried over Na₂SO₄, filtered and concentrated under vacuo. The crude was purified by column chromatography on silica gel (using a gradient of 0-100% EtOAc in isohexane as eluent) to afford the title compound as a colourless oil (95 mg, yield: 38%). ¹H-NMR (400 MHz, CDCl₃) δ 8.42 (s, 1H), 6.93 (s, 1H), 6.49 (t, J=55.6 Hz, 1H), 4.79 (s, 2H); purity: 95%.

### Intermediate NN247: 5-Amino-6-bromo-2-methoxy-3-pyridyl)methanol

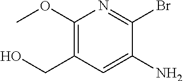

### Step 1: Synthesis of 2-methoxy-5-nitro-pyridine-3-carboxylic acid (NN248)

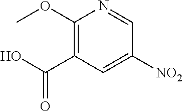

A solution of NaOMe, in MeOH (30%, 109 mL, 586 mmol) was added dropwise at 0° C. to a stirred solution of 2-chloro-5-nitro-pyridine-3-carboxylic acid (50 g, 235 mmol) in MeOH (500 mL) and cooled to 0° C. The reaction mixture was refluxed at 75° C. for 3 h. Solvent was removed under reduced pressure and the resulting residue was dissolved in 1M HCl aqueous solution (500 mL) and extracted with EtOAc (2×300 mL). Combined organic layers were dried over Na₂SO₄, filtered, and concentrated under vacuo to afford the title compound as pale yellow solid (41.6 g, 89%). LC-MS (Method 2) m/z [M+H]⁺: 199.2; rt: 1.01 min; purity: 99%. ¹H NMR (400 MHz, DMSO-d₆) δ 13.63 (brs, 1H), 9.22 (d, J=2.8 Hz, 1H), 8.74 (d, J=2.9 Hz, 1H), 4.07 (s, 3H).

### Step 2: Synthesis of ethyl 2-methoxy-5-nitro-pyridine-3-carboxylate (NN249)

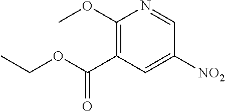 [SMILES: CCOC(=O)c1cc([N+](=O)[O-])cnc1OC]

To a stirred solution of 2-methoxy-5-nitro-pyridine-3-carboxylic acid NN248 (41.6 g, 199 mmol) in EtOH (500 mL) was added H₂SO₄ (11.7 g, 120 mmol) and the resulting solution was stirred at 90° C. overnight. Solvents were evaporated under reduced pressure and the resulting residue was dissolved in EtOAc (400 mL) and washed by saturated aqueous NaHCO₃ (2×250 mL). The organic phase was dried over Na₂SO₄, filtered and concentrated under reduced pressure to afford the title compound as a pale yellow solid (44 g, 91%). ¹H NMR (400 MHz, DMSO-d₆) δ 9.25 (d, J=2.8 Hz, 1H), 8.76 (d, J=2.8 Hz, 1H), 4.33 (q, J=7.1 Hz, 2H), 4.08 (s, 3H), 1.32 (t, J=7.1 Hz, 3H).

### Step 3: Synthesis of ethyl 5-amino-2-methoxy-pyridine-3-carboxylate (NN250)

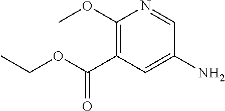

To a stirred solution of ethyl 2-methoxy-5-nitro-pyridine-3-carboxylate NN249 (44 g, 181 mmol) in EtOH (250 mL) was added Pd/C (5%, 19.3 g, 9.07 mmol) and the resulting suspension was stirred under hydrogen atmosphere (5 bar) for 16 h at 30° C. The reaction mixture was filtered through celite and the cake was washed with EtOH (250 mL). The filtrate was evaporated under reduced pressure to afford the title compound as pale red oil (30.2 g, 79%). ¹H NMR (400 MHz, DMSO-d₆) δ 7.71 (d, J=3.0 Hz, 1H), 7.42 (d, J=2.9 Hz, 1H), 5.01 (s, 2H), 4.23 (q, J=7.1 Hz, 2H), 3.77 (s, 3H), 1.27 (t, J=7.1 Hz, 3H).

### Step 4: Synthesis of ethyl 5-amino-6-bromo-2-methoxy-pyridine-3-carboxylate (NN251)

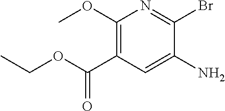

Ethyl 5-amino-2-methoxy-pyridine-3-carboxylate NN250 (30.2 g, 143 mmol) was dissolved in AcOH (200 mL) and NaOAc (11.8 g, 143 mmol) was added into this solution. The resulting reaction mixture was stirred at RT for 20 min. Br₂ (7.71 mL, 150 mmol) was added dropwise to the reaction mixture and the resulting dark brown solution was stirred at RT overnight. Solvents were evaporated and the residue was dissolved in EtOAc (700 mL) and washed with saturated aqueous NaHCO₃ (3×300 mL) followed by saturated aqueous Na₂S₂O₃ (2×300 mL). The organic layers were dried over Na₂SO₄, filtered and concentrated under reduced pressure. The resulting residue was purified by column chromatography on silica gel (using a gradient of 0-100% of EtOAc in isohexane as eluent) to afford the title compound as pale green solid (26.5 g, 64%). LC-MS m/z [M+H]⁺: 275.0/277.0; purity: 95%. ¹H NMR (400 MHz, DMSO-d₆) δ 7.62 (s, 1H), 5.19 (s, 2H), 4.25 (q, J=7.1 Hz, 2H), 3.79 (s, 3H), 1.27 (t, J=7.1 Hz, 3H).

### Step 5: Synthesis of (5-amino-6-bromo-2-methoxy-3-pyridyl)methanol (NN247)

To a stirred solution of ethyl 5-amino-6-bromo-2-methoxy-pyridine-3-carboxylate NN251 (26.5 g, 91.5 mmol) in THF (400 mL) was added LiBH₄ in THF (4.00 mol/L, 114 mL, 458 mmol) dropwise at 0° C. and the resulting solution was allowed to warm to RT and stirred for 16 h. The reaction mixture was diluted with brine (600 mL) and extracted with EtOAc (3×300 mL), the combined organic layers were dried over Na₂SO₄, filtered through an hydrophobic frit, and concentrated under reduced pressure to afford the title compound as a white solid (13.8 g, 63%). LC-MS m/z [M+H]⁺: 233.0, 235.0; purity: 100%. ¹H NMR (400 MHz, DMSO-d₆) δ 7.31-7.24 (m, 1H), 5.20 (t, J=5.0 Hz, 1H), 4.91 (s, 2H), 4.33 (d, J=4.4 Hz, 2H), 3.73 (s, 3H).

### Intermediate NN252: 4-Bromo-6-methoxy-5-methyl-pyridin-3-amine

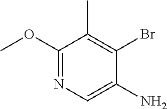

### Step 1: Synthesis of 4-bromo-2-methoxy-3-methyl-5-nitro-pyridine (N253)

To a solution of 4-bromo-2-methoxy-3-methyl-pyridine (1 g, 4.95 mmol) in conc. H₂SO₄ (7.14 mL) was added fuming HNO₃ (2.05 mL, 49.5 mmol) and the reaction mixture was stirred at RT overnight. The reaction mixture was poured into crushed ice, allowed to warm to RT then the precipitate was collected by filtration and dried under vacuum to afford the title compound as a beige solid (892 mg, 73%). LC-MS (Method 2) m/z [M+H]⁺: 247.0, 249.0; rt: 1.92 min; purity: 100%. ¹H NMR (400 MHz, DMSO-d₆) δ 8.73 (s, 1H), 4.01 (s, 3H), 2.33 (s, 3H).

### Step 2: Synthesis of 4-bromo-6-methoxy-5-methyl-pyridin-3-amine (NN252)

A suspension of 4-bromo-2-methoxy-3-methyl-5-nitro-pyridine NN253 (892 mg, 3.61 mmol) and Iron (1.61 g, 28.9 mmol) in AcOH (15 mL) was stirred at RT overnight. The reaction mixture was diluted with EtOAc (20 mL) then filtered through celite and washed with EtOAc (30 mL). The filtrate was collected then washed with 2.0 M aq. NaOH (3×50 mL). The organic layer was dried over Na₂SO₄ then the volatiles removed under vacuum to afford the title compound as a beige solid (635 mg, 80%). LC-MS m/z [M+H]⁺: 217.0, 219.0; purity: 99%. ¹H NMR (400 MHz, DMSO-d₆) δ 7.55 (s, 1H), 4.88 (s, 2H), 3.76 (s, 3H), 2.18 (s, 3H).

### Intermediate NN254: 4-Amino-3-bromo-6-(1-fluorocyclopropyl)pyran-2-one

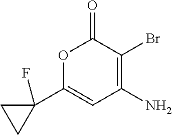 [SMILES: Nc1cc(C2(F)CC2)oc(=O)c1Br]

### Step 1: Synthesis of benzyl 1-fluorocyclopropanecarboxylate (NN255)

Triethylamine (13.4 mL, 96 mmol) and benzyl bromide (8.0 mL, 67 mmol) were added to a solution of 1-fluorocyclopropanecarboxylic acid (5 g, 48 mmol) in THF (50 mL), and the reaction mixture was stirred at RT overnight. DCM (50 mL) and aqueous HCl 1M (50 mL) were added to the reaction mixture. Phases were separated. The organic phase was washed with brine (10 mL) dried over Na₂SO₄, filtered and concentrated under reduced pressure. The resulting residue was purified by column chromatography on silica gel (using a gradient of 0-100% EtOAc in heptane as eluent) to afford the title compound as a colourless liquid (5.9 g, 63%). ¹H NMR (400 MHz, DMSO-de) δ 7.47-7.27 (m, 5H), 5.22 (s, 2H), 1.54-1.42 (m, 2H), 1.38-1.26 (m, 2H).

### Step 2: Synthesis of tert-butyl 5-(1-fluorocyclopropyl)-3,5-dioxo-pentanoate (NN256)

NaH (60%, 1.2 g, 30.3 mmol) was suspended in THF (50 mL) and cooled to 0° C. To this suspension was added tert-butyl 3-oxobutanoate NN255 (4.0 g, 25.3 mmol) slowly over 5 min under nitrogen and stirred further for 10 min at 0° C. To this mixture was added a solution n-BuLi in hexane (2.5 M, 11.2 mL, 28.1 mmol) over 5 minutes and allowed to stir for additional 10 min at 0° C., then methyl 1-fluorocyclopropanecarboxylate (5.8 g, 30 mmol) was added. The reaction mixture was allowed to warm up to RT and stirred for 2 h. The reaction mixture was cooled to 0° C. and neutralized with aqueous 1M HCl (30 mL). The solution was extracted with EtOAc (3×30 mL), the combined organic fractions were washed with brine (30 mL), dried over Na₂SO₄, and concentrated under reduced pressure to afford the title compound as a light orange liquid (5.2 g, 65%). LC-MS m/z [M+H-56]+: 189.1; purity: 77%.

### Step 3: Synthesis of 6-(1-fluorocyclopropyl)-4-hydroxy-pyran-2-one (NN257)

tert-butyl 5-(1-fluorocyclopropyl)-3,5-dioxo-pentanoate NN256 (5.2 g, 16.3 mmol) was slowly added to a solution of TFAA (12 mL) and TFA (4 mL) at 0° C. and then allowed to warm up to RT and stirred for 2 h. The reaction mixture was concentrated under reduced pressure and the resulting residue was purified by column chromatography on silica gel (using a gradient of 0-100% EtOAc in DCM as eluent) to afford the title compound as a pinkish solid (2.08 g, 67%). LC-MS m/z [M+H]⁺: 171.2; purity: 91%. ¹H NMR (400 MHz, DMSO-d₆) δ 11.93 (s, 1H), 6.22 (t, J=1.8 Hz, 1H), 5.28 (d, J=2.1 Hz, 1H), 1.58-1.44 (m, 2H), 1.38-1.25 (m, 2H).

### Step 4: Synthesis of 4-bromo-6-(1-fluorocyclopropyl)pyran-2-one (NN258)

6-(1-fluorocyclopropyl)-4-hydroxy-pyran-2-one NN257 (1.0 g, 5.3 mmol), tetrabutylammonium bromide (2.05 g, 6.35 mmol) and P₂O₅ (1.73 g, 6.1 mmol) were suspended in toluene (40 mL) and the reaction mixture was stirred at 95° C. for 1 h. The reaction mixture was cooled to RT and saturated aqueous NaHCO₃ (20 mL) was carefully added. The layers were separated, and the aqueous phase was extracted with EtOAc (20×3 mL). The organics were combined, washed with brine (10 mL), dried over Na₂SO₄, filtered, and concentrated under vacuo. The resulting residue was purified by column chromatography on silica gel (using a gradient of 0-100% EtOAc in isohexane as eluent) to afford the title compound as a light yellow crystallin solid (900 mg, 65%). LC-MS m/z [M+H]⁺: 233.0, 235.0; purity: 89%.

### Step 5: Synthesis of 4-azido-6-(1-fluorocyclopropyl)pyran-2-one (NN259)

4-bromo-6-(1-fluorocyclopropyl)pyran-2-one NN258 (900 mg, 3.43 mmol) was dissolved in DMF (10 mL) and NaN₃ (312 mg, 4.80 mmol) was added. The reaction mixture was stirred at RT overnight. The reaction mixture was poured into ice cold water (30 mL) and the aqueous phase was extracted with TBME (3×30 mL). The combined organic phases were washed with brine (2×20 mL), dried over Na₂SO₄, filtered, and concentrated under vacuo to afford the title compound as a light yellow solid (650 mg, 88%). LC-MS m/z [M+H]⁺: 196.1; purity: 91%.

### Step 6: Synthesis of 4-amino-6-(1-fluorocyclopropyl)pyran-2-one (NN260)

4-azido-6-(1-fluorocyclopropyl)pyran-2-one NN259 (650 mg, 3.02 mmol) was dissolved in EtOH (20 mL) and Pd/C (10%, 161 mg) was added. The reaction mixture was stirred at RT under hydrogen atmosphere (1 bar) for 1 h. The reaction mixture was filtered and concentrated under vacuo to afford the title compound as a yellowish sticky solid (592 mg, 98%). LC-MS m/z [M+H]⁺: 170.1; purity: 100%. ¹H NMR (400 MHz, DMSO-de) δ 7.01 (s, 2H), 6.16-6.07 (m, 1H), 4.85-4.79 (m, 1H), 1.54-1.37 (m, 2H), 1.30-1.17 (m, 2H).

### Step 7: Synthesis of 4-amino-3-bromo-6-(1-fluorocyclopropyl)pyran-2-one (NN254)

4-amino-6-(1-fluorocyclopropyl)pyran-2-one NN260 (630 mg, 3.17 mmol) was dissolved in DCM (15 mL) and MeCN (15 mL). NBS (563 mg, 3.17 mmol) was added and the reaction mixture was stirred at RT for 1 h. The reaction mixture was concentrated under vacuum and the crude was purified by column chromatography on silica gel (using a gradient of 0-100% of 3:1 EtOAc-EtOH in isohexane) to afford the title compound as a beige solid (790 mg, 98%). LC-MS m/z [M+H]⁺: 248.0, 250.0; purity: 98%.

### Intermediate NN261: 2-Bromo-5-(difluoromethyl)-6-methoxy-pyridin-3-amine

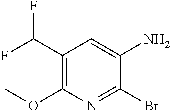

### Step 1: Synthesis of N-[5-(difluoromethyl)-6-methoxy-3-pyridyl]-1,1-diphenyl-methanimine (NN262)

To a solution of 5-bromo-3-(difluoromethyl)-2-methoxy-pyridine (900 mg, 3.78 mmol) in 1,4-dioxane (30.0 mL) were added Cs₂CO₃ (3.08 g, 9.45 mmol), BINAP (235 mg, 0.38 mmol) and Pd₂(dba)₃ (217 mg, 0.38 mmol) under nitrogen. Diphenylmethanimine (1.7 g, 5.67 mmol) was added and the mixture stirred at 100° C. for 8 h then cooled to RT. EtOAc (50 mL) was added to the reaction mixture followed by water (25 mL). The aqueous phase was separated, and the remaining organic phase was washed with brine (25 mL). The organic phase was dried over Na₂SO₄, filtered, and concentrated under vacuo. The crude was purified by column chromatography on silica gel (using a gradient of 0-50% EtOAc in isohexane as eluent) to afford the title compound as a light-yellow gum (1.52 g, 80%). LC-MS m/z [M+H]⁺: 339.1; purity: 67%.

### Step 2: Synthesis of 5-(difluoromethyl)-6-methoxy-pyridin-3-amine (NN263)

N-[5-(difluoromethyl)-6-methoxy-3-pyridyl]-1,1-diphenyl-methanimine NN262 (67%, 1.52 g, 3.03 mmol) was dissolved in a mixture of THF (15 mL) and water (3.0 mL) and treated with aqueous HCl 1 M (6 mL). The reaction mixture was stirred at RT for 2 h. The reaction was then concentrated and purified by SCX cartridge (using MeOH then MeOH+0.7 M NH₃ as eluent). The ammonia fraction was concentrated under vacuo to afford the title compound as a white powder (516 mg, 97%). LC-MS m/z [M+H]⁺: 175.1; purity: 97%.

### Step 3: Synthesis of 2-bromo-5-(difluoromethyl)-6-methoxy-pyridin-3-amine (NN261)

5-(difluoromethyl)-6-methoxy-pyridin-3-amine NN263 (516 mg, 2.87 mmol) was dissolved in DMF (10.0 mL) then NBS (563 mg, 3.16 mmol) was added. The reaction mixture was stirred at RT for 1 h. The reaction mixture was then treated with EtOAc (50 mL). The organic phase was separated and washed with aqueous LiCl 1M (2×20 mL) then with brine (2×20 mL). The organic phase was dried over Na₂SO₄, filtered, and concentrated under vacuo. The crude was purified by column chromatography on silica gel (using a gradient of 0-60% EtOAc in isohexane as eluent) to afford the title compound as a light brown solid (641 mg, 88%). LC-MS m/z [M+H]⁺: 252.9, 255.0; purity: 100%.

### Intermediate NN264: 3-Bromo-2-methoxy-6-[(4-methoxyphenyl)methoxy]pyridin-4-amine

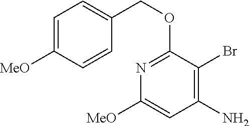 [SMILES: COc1ccc(COc2nc(OC)cc(N)c2Br)cc1]

### Step 1: Synthesis of 2-fluoro-6-[(4-methoxyphenyl)methoxy]pyridin-4-amine (NN265)

To a solution of 4-Methoxybenzyl alcohol (5.37 g, 38.9 mmol) in DMF (80 mL) was added portion wise NaH (60%, 933 mg, 38.9 mmol) and the reaction mixture was stirred at RT for 30 min. To this solution was added 2,6-difluoropyridin-4-amine (5.00 g, 38.4 mmol) and the reaction mixture was stirred for 1 h at 80° C. The reaction mixture was allowed to cool down to RT, poured onto water (200 mL) and the solid was collected by filtration and dried. The crude was purified by column chromatography on silica gel (using a gradient of 0-80% TBME in isohexane as eluent) to afford the title compound as an off white solid (5.3 g, 56%). LC-MS m/z [M+H]⁺: 249.2; purity: 92%. ¹H NMR (400 MHz, DMSO-d₆) δ 7.37-7.27 (m, 2H), 6.96-6.86 (m, 2H), 6.34 (s, 2H), 5.75 (s, 2H), 5.08 (s, 2H), 3.75 (s, 3H). ¹⁹F NMR (376 MHz, DMSO-d₆) δ−73.72.

### Step 2: Synthesis of 2-methoxy-6-[(4-methoxyphenyl)methoxy]pyridin-4-amine (NN266)

A mixture of 2-fluoro-6-[(4-methoxyphenyl)methoxy]pyridin-4-amine NN265 (1.0 g, 4.03 mmol) and a solution of NaOMe in MeOH (5.4 M, 2.97 mL, 16.1 mmol) in MeOH (15 mL) was heated at 120° C. under microwave for 5 h. The reaction mixture was allowed to cool down to RT and the solvent was removed under vacuo. The residue was triturated with water (40 mL). The solid was collected, washed with water (50 mL). The process was repeated 5 times. The solids were collected and combined to afford the title compound (4.73 g, 88%). LC-MS m/z [M+H]⁺: 261.2; purity: 98%.

### Step 3: Synthesis of 3-bromo-2-methoxy-6-[(4-methoxyphenyl)methoxy]pyridin-4-amine (NN264)

To a solution of 2-methoxy-6-[(4-methoxyphenyl)methoxy]pyridin-4-amine NN266 (4.50 g, 16.9 mmol) in DCM (300 mL) stirred at 0° C. was added portion wise NBS (3.3 g, 18.6 mmol) over 60 min and the reaxtion mixture was stirred for a further 30 min. The reaction mixture was evaporated to dryness and the crude was purified by column chromatography on silica gel (using a gradient of 0-30% TBME in isohexane as eluent) to afford the title compound as a white solid (2.46 g, 41%).

¹H NMR (400 MHz, DMSO-d₆) δ 7.36-7.28 (m, 2H), 6.93-6.89 (m, 2H), 6.10 (s, 2H), 5.75 (s, 1H), 5.16 (s, 2H), 3.82 (s, 3H), 3.74 (d, J=1.6 Hz, 3H).

### Intermediate NN267: 3,5-Difluoro-2-(4,4,5,5-tetramethyl-1,3,2-dioxaborolan-2-yl)amine

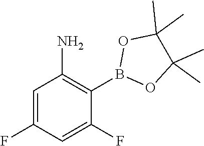 [SMILES: CC1(C)OB(c2c(N)cc(F)cc2F)OC1(C)C]

A suspension of 2-bromo-3,5-difluoro-aniline (CAS: 500357-40-4) (500 mg, 2.40 mmol), 4,4,5,5-tetramethyl-2-(4,4,5,5-tetramethyl-1,3,2-dioxaborolan-2-yl)-1,3,2-dioxaborolane (1.20 g, 4.73 mmol) and K₂CO₃ (1.5 g, 15.3 mmol) in 1,4-dioxane (5 mL) was degassed with nitrogen for 5 min. Pd(dppf)Cl₂ (176 mg, 0.24 mmol) was added and the reaction mixture was degassed for 5 min. The reaction mixture was stirred at reflux overnight. After cooling to RT, the reaction mixture was diluted with EtOAc (20 mL) and filtered through celite. The filter pad was washed with EtOAc (20 mL) and the solvent was removed under vacuo. The crude was purified by column chromatography on silica gel (using a gradient of 0-100% MTBE in isohexane as eluent) to afford the title compound as a pale yellow oil that solidified upon standing (945 mg, yield: 100%). ¹H NMR (400 MHz, DMSO-d₆) δ 6.51-6.37 (m, 1H), 6.21 (ddd, J=11.9, 2.3, 1.0 Hz, 1H), 6.15 (s, 2H), 1.28 (s, 12H).

### Intermediate NN268: 4-Amino-3-bromo-6-methyl-pyran-2-one

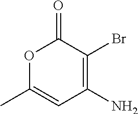

### Step 1: Synthesis of 4-bromo-6-methyl-pyran-2-one (NN269)

4-hydroxy-6-methyl-pyran-2-one (5 g, 39.6 mmol), tetrabutylammonium bromide (15.3 g, 47.6 mmol) and P₂O₅ (13 g, 45.6 mmol) were suspended in toluene (150 mL) and the reaction mixture was stirred at 95° C. for 1 h. The reaction mixture was cooled down to RT and saturated aqueous NaHCO₃ (500 mL) was carefully added. The layers were separated, and the aqueous phase was extracted with EtOAc (300 mL). The organics were combined, washed with saturated aqueous NaHCO₃ (300 mL), dried over Na₂SO₄, filtered, and concentrated under vacuo to afford the title compound as a brown solid (4.1 g, 54%). ¹H NMR (400 MHz, CDCl₃) δ 6.46 (dd, J=1.7, 0.8 Hz, 1H), 6.19 (dd, J=1.7, 0.9 Hz, 1H), 2.25 (t, J=0.8 Hz, 3H).

### Step 2: Synthesis of 4-azido-6-methyl-pyran-2-one (NN270)

4-bromo-6-methyl-pyran-2-one NN269 (4 g, 21.2 mmol) was dissolved in DMF (50 mL) and NaN₃ (2.06 g, 31.7 mmol) was added. The reaction mixture was stirred at RT for 1 h. The reaction mixture was poured into ice cold water (300 mL) and the aqueous phase was extracted with TBME (3×100 mL). The combined organics were washed with brine (2×100 mL), dried over Na₂SO₄, filtered, and concentrated under vacuo to afford the title compound as an orange solid (2.7 g, 78%). ¹H NMR (400 MHz, DMSO-d₆) δ 6.21 (dd, J=2.0, 1.0 Hz, 1H), 5.87-5.79 (m, 1H), 2.22 (t, J=0.8 Hz, 3H).

### Step 3: Synthesis of 4-amino-6-methyl-pyran-2-one (NN271)

4-azido-6-methyl-pyran-2-one NN270 (2.7 g, 16.5 mmol) was dissolved in EtOH (50 mL) and Pd/C (5%, 1.76 g, 0.83 mmol) was added. The reaction mixture was stirred at RT under hydrogen atmosphere (3 bar) for 3 h. The reaction mixture was filtered and concentrated under vacuo to afford the title compound as a beige solid (2.1 g, 96%). ¹H NMR (400 MHz, DMSO-d₆) δ 6.78 (s, 2H), 5.72 (dd, J=2.0, 1.0 Hz, 1H), 4.77 (d, J=1.9 Hz, 1H), 2.07 (d, J=0.9 Hz, 3H).

### Step 4: Synthesis of 4-amino-3-bromo-6-methyl-pyran-2-one (NN268)

4-amino-6-methyl-pyran-2-one NN271 (150 mg, 1.14 mmol) was suspended in DCM/MeCN (1:1, 10.0 mL) and Br₂ (0.064 mL, 1.3 mmol) was added. The reaction mixture was stirred at RT for 1 h. The reaction mixture was concentrated under vacuo and the crude was purified by column chromatography on silica gel (using a gradient of 0-5% MeOH in DCM as eluent) to afford the title compound as a light orange solid (161 mg, 69%). ¹H NMR (400 MHz, DMSO-d₆) δ 7.56 (s, 1H), 6.67 (s, 1H), 5.89 (d, J=1.0 Hz, 1H), 2.10 (d, J=0.9 Hz, 3H).

### Intermediate NN272: 3-Fluoro-5-methoxy-2-(4,4,5,5-tetramethyl-1,3,2-dioxaborolan-2-yl)aniline

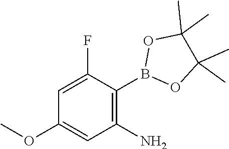

Under nitrogen, to a solution of 3-fluoro-5-methoxy-aniline (CAS 2339-58-4) (1.3 g, 9.2 mmol) in THE (19 mL) was added 4,4,5,5-tetramethyl-2-(4,4,5,5-tetramethyl-1,3,2-dioxaborolan-2-yl)-1,3,2-dioxaborolane (3.5 g, 0.014 mol), (1,5-cyclooctadiene)(methoxy)iridium(I) dimer (0.12 g, 0.18 mmol) and 4,4′-di-tert-butyl-2,2′-bipyridine (0.11 g, 0.4 mmol). The reaction mixture was heated at reflux overnight then cooled to RT. The reaction mixture was filtered through a pad of celite and washed with EtOAc (100 mL) then concentrated under reduced pressure. The filtrate was concentrated under vacuum. The crude was purified by column chromatography on silica gel (using a gradient of 0-50% EtOAc in isohexane as eluent) to afford the title compound as an ochre solid (440 mg, yield: 10%). LC-MS m/z [M-C₆H₁₀+H]⁺: 186.2; purity: 69%. ¹H NMR (400 MHz, DMSO-d₆) δ 5.97 (d, J=2.2 Hz, 1H), 5.88 (s, 2H), 5.82 (dd, J=12.1, 2.2 Hz, 1H), 3.67 (s, 3H), 1.26 (s, 12H). ¹⁹F NMR (376 MHz, DMSO-d₆) δ−99.19.

### Intermediate NN273: 4-Amino-3-bromo-1-methyl-pyridin-2-one

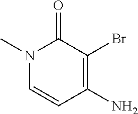

To a stirred solution of 1,4-dimethylpyridin-2-one (500 mg, 4.06 mmol) in MeCN (10.0 mL) was added NBS (723 mg, 4.06 mmol) portion wise and stirred at RT for 3 h. The volatiles were removed under reduced pressure, and the crude was purified by column chromatography on silica gel (using a gradient of 0-10% 0.7 M NH₃ MeOH in DCM as eluent) to afford the title compound (457 mg, yield: 56%) as an off white solid. LC-MS m/z [M+H]⁺: 203.0, 205.1; purity: 100%. ¹H NMR (400 MHz, DMSO-d₆) δ 7.35 (d, J=7.4 Hz, 1H), 6.24 (s, 2H), 5.83 (d, J=7.4 Hz, 1H), 3.31 (s, 3H).

### Intermediate NN274: 3-Bromo-2-methoxy-6-methyl-pyridin-4-amine

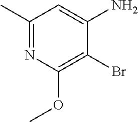

To a solution of 2-methoxy-6-methyl-pyridin-4-amine (5.0 g, 36.2 mmol) in DCM (120 mL) at 0° C. was added NBS (6.8 g, 38.0 mmol) portion wise over 30 minutes. The reaction mixture was stirred at 0° C. for 1 h and then water (150 mL) was added. The phases were separated, and the aqueous layer was extracted with DCM (2×80 mL). The combined extracts were dried over Na₂SO₄, filtered, and concentrated under vacuum. The crude was purified by column chromatography on silica gel (using a gradient of 0-2% 0.7 M NH₃ MeOH in DCM as eluent) to afford the title compound as a colourless oil (6.8 g, yield: 86%). LC-MS m/z [M+H]⁺: 217.1, 219.0; purity: 100%. ¹H NMR (400 MHz, CDCl₃) δ 6.14 (d, J=3.1 Hz, 1H), 4.47 (s, 2H), 3.95 (s, 3H), 2.29 (d, J=2.1 Hz, 3H).

### Intermediate NN275: 4-Amino-5-bromo-2-methyl-benzonitrile

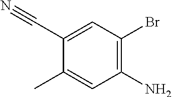

### Step 1: Synthesis of 4-amino-2-methyl-benzonitrile (NN276)

A mixture of 2-methyl-4-nitro-benzonitrile (3 g, 18.5 mmol), iron (2.07 g, 37.0 mmol) and acetic acid (150 mL) was heated and stirred at 90° C. for 30 minutes. The reaction mixture was allowed to cool to rt and then filtered through a plug of celite. The filtrate was concentrated to dryness and the residue taken up into EtOAc (100 mL) and the organics washed with saturated NaHCO₃ solution (100 mL). The organic phase was separated, dried over Na₂SO₄ and concentrated under vacuum. The crude was purified by column chromatography on silica gel (using a gradient of 0-70% EtOAc in isohexane as eluent) to afford the title compound as a pale yellow solid (2.35 g, yield: 94%). LC-MS m/z [M+H]⁺: 133.2; purity: 98%. ¹H NMR (400 MHz, DMSO-d₆) δ 7.30 (d, J=8.4 Hz, 1H), 6.49-6.39 (m, 2H), 6.03 (s, 2H), 2.27 (s, 3H).

### Step 2: Synthesis of 4-amino-5-bromo-2-methyl-benzonitrile (NN275)

NBS (673 mg, 3.78 mmol) was added portion wise over 15 minutes to a stirred solution of 4-amino-2-methyl-benzonitrile NN276 (500 mg, 3.78 mmol) in MeCN (10.0 mL). Upon completion of the addition the reaction mixture was stirred at RT for a further 1 h before being poured into water (50 mL). The crude product was extracted with EtOAc (2×20 mL). The combined organic extracts were washed with water (20 mL) and dried over Na₂SO₄, filtered, and concentrated under vacuum to afford the title compound as an off-white solid (380 mg, yield: 24%). LC-MS m/z [M+H]⁺: 211.1, 213.1; purity: 50%. ¹H NMR (400 MHz, DMSO-d₆) δ 7.72 (s, 1H), 6.31 (s, 1H), 6.24 (s, 2H), 2.27 (s, 3H).

### Intermediate NN235: 3-bromo-2-(difluoromethyl)-6-methyl-pyridin-4-amine

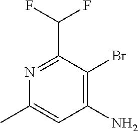

Under argon a solution of bis(difluoroacetoxy)iodo)benzene (3.0 g, 7.6 mmol) and tert-butyl N-(5-bromo-2-methyl-4-pyridyl)-N-tert-butoxycarbonyl-carbamate (1.0 g, 2.582 mmol) in dry dichloromethane (12.9 mL) and difluoroacetic acid (0.1625 mL, 2.582 mmol) was prepared. Using a Vapourtec E-series flow system equipped with a 10 mL UV-150 reactor (365 nm LED light source) and conditioned using dry dichloromethane, the solution was pumped at a flow rate of 0.5 mL/min and irradiated for 20 min. Exiting the reactor the reaction mixture was collected and the reactor was washed using dry dichloromethane. The collected layer was concentrated under vacuo to give an orange oil. The orange oil was diluted in dichloromethane (1.5 mL) and trifluoroacetic acid (1.5 mL, 20 mmol) was slowly added to the orange solution. After 5 h stirring at RT, the reaction mixture was treated with sat. NaHCO₃ solution (50 mL) and extracted with isopropylacetate (100 mL+2×50 mL). The combined organic extracts were washed with brine (100 mL), dried over sodium sulfate, filtered, and concentrated under vacuum, then purified by reverse phase chromatography (Method P_B) to give the title compound (631 mg, 1.28 mmol, yield: 50%) as an off-white solid. LC-MS m/z: [M+H]⁺: 237/239; purity: 96.6%. ¹H NMR (400 MHz, DMSO-d₆) δ 6.90 (t, J=54.0 Hz, 1H), 6.63 (s, 1H), 6.49 (s, 2H), 2.28 (s, 3H).

### Intermediate NN277: 5-Bromo-2-(1-fluorocyclopropyl)pyridin-4-amine

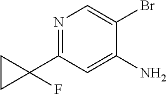 [SMILES: Nc1cc(C2(F)CC2)ncc1Br]

### Step 1: Synthesis of 1-fluorocyclopropanecarbonyl chloride (NN278)

Oxalyl chloride (3.86 mL, 45.0 mmol) was added to a solution of 1-fluorocyclopropanecarboxylic acid (4.68 g, 45.0 mmol) and DMF (0.100 mL, 1.30 mmol) in DCM (100 mL) at 0° C. and the reaction mixture was stirred at RT for 3 h. The solution was concentrated under reduced pressure to afford the title compound as a colourless oil (6.14 g). The product was used in the next step without further purification.

### Step 2: Synthesis of (1Z,4E)-1-(1-fluorocyclopropyl)-1-hydroxy-5-methoxy-penta-1,4-dien-3-one (NN279)

To a solution of (E)-4-methoxybut-3-en-2-one (10.0 g, 90.2 mmol) in THF (120 mL) at −70° C. was added dropwise a solution of LiHMDS 1 M in THF (90.2 mL, 90.2 mmol). The resulting red solution was stirred at −70° C. for 20 min then a solution of 1-fluorocyclopropanecarbonyl chloride NN278 (6.14 g, 45.1 mmol) in THF (80 mL) was added over 20 min. The resulting solution was stirred at −70° C. for 1 h then let warmed at RT overnight. A saturated aqueous solution of NH₄Cl (250 mL) was added and was extracted with EtOAc (3×200 mL). The combined organic extracts were washed with brine (250 mL), dried over Na₂SO₄, filtered and concentrated under reduced pressure to afford the title compound as a red oil (11.5 g, yield: quant.). LC-MS m/z [M+H]⁺: 187.1; purity: 73%.

### Step 3: Synthesis of 2-(1-fluorocyclopropyl)pyran-4-one (NN280)

TFA (10.3 g, 90.2 mmol) was added to a solution of (1Z,4E)-1-(1-fluorocyclopropyl)-1-hydroxy-5-methoxy-penta-1,4-dien-3-one NN279 (73%, 11.5 g, 45 mmol) in DCM (60 mL). The solution was stirred at RT for 2 h then concentrated under reduced pressure. The crude was purified by column chromatography on silica gel (using a gradient of 0-100% EtOAc in isohexane as eluent) to afford the title compound as a red solid (2.44 g, yield: 32%). LC-MS m/z [M+H]⁺: 155.0; purity: 98%. ¹H NMR (400 MHz, DMSO-d₆) δ 8.10 (d, J=5.8 Hz, 1H), 6.39 (dd, J=2.6, 1.5 Hz, 1H), 6.29 (dd, J=5.8, 2.5 Hz, 1H), 1.60-1.50 (m, 2H), 1.40-1.32 (m, 2H); purity: 90%.

### Step 4: Synthesis of 2-(1-fluorocyclopropyl)-1H-pyridin-4-one (NN281)

A solution of 2-(1-fluorocyclopropyl)pyran-4-one NN280 (2.44 g, 14.2 mmol) and aqueous NH₄OH (35%, 27 mL, 243 mmol) was heated at 50° C. for 1 h then concentrated under reduced pressure to afford the title compound as a red gum (2.35 g, yield: 100%). LC-MS m/z [M+H]⁺: 154.2; purity: 98%.

### Step 5: Synthesis of 4-bromo-2-(1-fluorocyclopropyl)pyridine (NN282)

A mixture of 2-(1-fluorocyclopropyl)pyridin-4-ol NN281 (2.35 g, 14.3 mmol) and POBr₃ (4.5 g, 15.7 mmol) was heated at 120° C. for 1 h then cooled to RT. A saturated aqueous solution of NaHCO₃ (200 mL) was added and was extracted with EtOAc (3×100 mL). The combined organic extracts were washed with brine (200 mL), dried over Na₂SO₄, filtered and concentrated under reduced pressure. The crude was purified by column chromatography on silica gel (using a gradient of 0-100% EtOAc in isohexane as eluent) to afford the title compound as a pale beige solid (1.97 g, yield: 64%). ¹H NMR (400 MHz, DMSO-d₆) δ 8.40 (dd, J=5.3, 1.2 Hz, 1H), 7.77 (t, J=1.8 Hz, 1H), 7.59 (dd, J=5.3, 1.9 Hz, 1H), 1.60-1.54 (m, 1H), 1.54-1.49 (m, 1H), 1.38-1.35 (m, 1H), 1.35-1.31 (m, 1H); purity: 100%.

### Step 6: Synthesis of tert-butyl N-[2-(1-fluorocyclopropyl)-4-pyridyl]carbamate (NN283)

A suspension of 4-bromo-2-(1-fluorocyclopropyl)pyridine NN282 (1.86 g, 8.61 mmol), tert-butyl carbamate (2.02 g, 17.2 mmol), Cs₂CO₃ (5.61 g, 17.2 mmol) and XPhos Pd(crotyl)Cl (290 mg, 0.430 mmol) in 1,4-dioxane (79 mL) was refluxed under N₂ overnight then cooled to rt. More tert-butyl carbamate (500 mg), Cs₂CO₃ (1.80 g) and XPhos Pd(crotyl)Cl (100 mg) were added and the reaction mixture was again refluxed overnight then cooled to RT. The suspension was filtered on a pad of celite and washed with EtOAc (150 mL). The filtrate was concentrated under reduced pressure, and the crude was purified by column chromatography on silica gel (using a gradient of 0-50% EtOAc in isohexane as eluent) to afford the title compound as an orange solid (2.07 g, yield: 65%). LC-MS m/z [M+H]⁺: 253.2; purity: 98%. ¹H NMR (400 MHz, DMSO-d₆) δ 9.91 (s, 1H), 8.25 (dd, J=5.6, 1.2 Hz, 1H), 7.74 (t, J=2.0 Hz, 1H), 7.30 (dd, J=5.6, 2.1 Hz, 1H), 1.49 (s, 9H), 1.47-1.44 (m, 1H), 1.42 (q, J=5.0 Hz, 1H), 1.32-1.29 (m, 1H), 1.29-1.25 (m, 1H); purity: 68%.

### Step 7: Synthesis of 2-(1-fluorocyclopropyl)pyridin-4-amine (NN284)

A suspension of tert-butyl N-[2-(1-fluorocyclopropyl)-4-pyridyl]carbamate NN283 (68%, 2.07 g, 5.58 mmol) in 4 M HCl in 1,4-dioxane (27.9 mL, 112 mmol) and 1,4-dioxane (27.7 mL) was stirred at RT overnight. The resulting suspension was filtered, and the precipitate was washed with TBME (100 mL) then dried under reduced pressure. The resulting solid was dissolved in MeOH (15 mL) and loaded on a SCX column (20 g). The column was eluted first with MeOH (100 mL) which was discarded then with 0.7 M NH₃ in MeOH (100 mL). The filtrate was concentrated to afford the title compound as a pale beige solid (730 mg, yield: 85%). LC-MS m/z [M+H]⁺: 153.2; purity: 99%. ¹H NMR (400 MHz, DMSO-d₆) δ 7.87 (d, J=5.6 Hz, 1H), 6.72 (t, J=2.2 Hz, 1H), 6.32 (ddd, J=5.5, 2.3, 0.9 Hz, 1H), 6.11 (s, 2H), 1.39-1.34 (m, 1H), 1.34-1.29 (m, 1H), 1.28-1.18 (m, 2H).

### Step 8: Synthesis of 5-bromo-2-(1-fluorocyclopropyl)pyridin-4-amine (NN277)

To a solution of 2-(1-fluorocyclopropyl)pyridin-4-amine NN284 (675 mg, 4.39 mmol) in MeCN (45.0 mL) at 0° C. was added NBS (782 mg, 4.4 mmol). The solution was stirred at 0° C. for 2 h then warmed to RT and stirred overnight. MeCN was removed under vacuum, water (25 mL) was then added and was extracted with EtOAc (3×25 mL). The combined organic extracts were washed with brine (50 mL), dried over Na₂SO₄, filtered and concentrated under vacuum. The crude was purified by column chromatography on silica gel (using a gradient of 0-70% EtOAc in isohexane as eluent) to afford the title compound as a beige solid (623 mg, yield: 49%). LC-MS m/z [M+H]⁺: 231.0, 233.1; purity: 93%. ¹H NMR (400 MHz, DMSO-d₆) δ 8.12 (d, J=1.3 Hz, 1H), 6.93 (d, J=2.0 Hz, 1H), 6.41 (s, 2H), 1.44-1.39 (m, 1H), 1.39-1.33 (m, 1H), 1.26-1.19 (m, 2H); purity: 85%.

### Intermediate NN285 2-bromo-6-methoxy-5-methyl-pyridin-3-amine

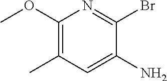

6-methoxy-5-methyl-pyridin-3-amine (10 g, 72.4 mmol) was dissolved in AcOH (100 mL) and CH₃COONa (5937 mg, 72.4 mmol) was added. The reaction mixture was stirred at RT for 20 minutes. A solution of Br₂ (3.89 mL, 76.0 mmol) in AcOH (50 mL) was slowly added dropwise to the reaction mixture and the resulting dark brown solution was stirred at RT for 2 h. The reaction mixture was slowly poured into a stirring solution of crushed ice (250 g) and 2 N NaOH (100 mL) and stirred at 0° C. for 30 minutes. This mixture was then neutralised using NaOH (aq, 6N). The solid thus formed was collected and dried under vacuo to afford a dark brown solid. The crude was purified by column chromatography on silica gel (using a gradient of 0-100% EtOAc in isohexane as eluent) to afford the title compound as a brown solid (8.85 g, yield: 51%). LC-MS m/z [M+H]⁺: 217.0, 219.1; purity: 91%. ¹H NMR (400 MHz, DMSO-d₆) δ 7.03 (d, J=1.0 Hz, 1H), 4.81 (s, 2H), 3.74 (s, 3H), 2.02 (d, J=0.9 Hz, 3H).

### Intermediate NN286: 5-Chloro-3-fluoro-2-(4,4,5,5-tetramethyl-1,3,2-dioxaborolan-2-yl)aniline

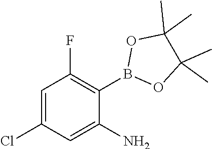 [SMILES: CC1(C)OB(c2c(N)cc(Cl)cc2F)OC1(C)C]

A solution of 3-chloro-5-fluoro-aniline (0.50 g, 3.43 mmol),4,4,5,5-tetramethyl-2-(4,4,5,5-tetramethyl-1,3,2-dioxaborolan-2-yl)-1,3,2-dioxaborolane (959 mg, 3.78 mmol), 4-tert-butyl-2-(4-tert-butyl-2-pyridyl)pyridine (87 mg, 0.32 mmol) and (1,5-cyclooctadiene)(methoxy)iridium(I) dimer (87 mg, 0.13 mmol) in dry THF (10.0 mL) was heated at 150° C. for 20 h. The volatiles were removed under vacuum and the crude product was purified by column chromatography on silica gel (using a gradient of 0-60% MTBE in isohexane as eluant) to afford the title compound as a colourless solid (346 mg, yield: 36%). LC-MS m/z [M-C₆H₁₀+H]⁺: 190.2, 192.2; purity: 95%. ¹H NMR (400 MHz, DMSO-d₆) δ 6.49 (d, J=1.8 Hz, 1H), 6.28 (dd, J=9.7, 1.8 Hz, 1H), 6.11 (s, 2H), 1.28 (s, 12H).

### Intermediate NN440: 2-bromo-5-cyclopropyl-6-methoxy-pyridin-3-amine

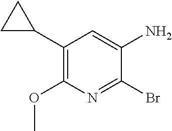

### Step-1: Synthesis of 3-cyclopropyl-2-methoxy-5-nitro-pyridine

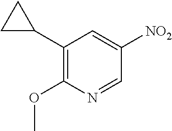

3-bromo-2-methoxy-5-nitro-pyridine (2.0 g, 8.58 mmol), Cs₂CO₃ (5.6 g, 17 mmol), cyclopropylboronic acid (885 mg, 10.3 mmol), Tetrakis(triphenylphosphine)palladium(0) (496 mg, 0.43 mmol), were suspended in 1,4-dioxane (30 ml). The reaction mixture was degassed then stirred at reflux under nitrogen overnight. The reaction was cool to room temperature then filtered and concentrated. The crude was purified by column chromatography on silica gel (using a gradient, 0-100% EtOAc in isohexane as eluant) to afford the title compound as a white powder (1.1 g, yield: 62%). ¹H NMR (400 MHz, DMSO-d₆) δ 8.89 (d, J=2.7 Hz, 1H), 7.94 (dd, J=2.7, 0.6 Hz, 1H), 4.05 (s, 3H), 2.10-2.02 (m, 1H), 1.03-0.97 (m, 2H), 0.85-0.80 (m, 2H); purity 95%

### Step-2: Synthesis of 5-cyclopropyl-6-methoxy-pyridin-3-amine

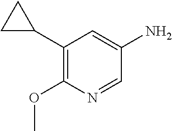

3-cyclopropyl-2-methoxy-5-nitro-pyridine (1.1 g, 5.5 mmol) was dissolved in EtOH (30 mL) and the mixture was purged with nitrogen. Pd/C 10% (603 mg) was added, the mixture was purged with H₂ then stirred under a H₂ atmosphere (1 bar) overnight. The catalyst was filtered off and the filtrate concentrated to afford the title compound as a dark purple oil (894 mg, yield: 98%). LC-MS (Method 5) m/z [M+H]⁺: 165.2; rt: 0.285 min; purity: 98%. ¹H NMR (400 MHz, DMSO-d₆) δ 7.35 (d, J=2.7 Hz, 1H), 6.61 (d, J=2.7 Hz, 1H), 5.27 (s, 2H), 3.77 (s, 3H), 1.94 (tt, J=8.4, 5.3 Hz, 1H), 0.92-0.83 (m, 2H), 0.59-0.51 (m, 2H).

### Step-3: Synthesis of 2-bromo-5-cyclopropyl-6-methoxy-pyridin-3-amine NN440

5-cyclopropyl-6-methoxy-pyridin-3-amine (894 mg, 5.4 mmol) was dissolved in AcOH (10 mL) and CH₃COONa (439 mg, 5.4 mmol) was added. The reaction mixture was stirred at RT for 20 minutes. Br₂ (0.287 mL, 5.6 mmol) in AcOH (5 mL) was slowly added dropwise to the reaction mixture and the resultant dark brown solution was stirred at RT for 3 h. The reaction mixture was slowly poured into a stirring solution of crushed ice (250 g) and stirred at 0° C. for 30 minutes. This mixture was then neutralised using NaOH (aq, 6N). and extracted with DCM (3×150 mL), the combined organics washed with brine (100 mL), dried using phase Sep cartridge and evaporated to dryness. The crude was purified by column chromatography on silica gel (using a gradient, 0-30% EtOAc in isohexane as eluant) to afford the title compound as a dark brown oil (526 mg, yield: 40%). LC-MS (Method 5) m/z [M+H]⁺: 243.0, 245.0; rt: 1.72 min; purity: 99%. ¹H NMR (400 MHz, DMSO-d₆) δ 6.75 (s, 1H), 4.75 (s, 2H), 3.76 (s, 3H), 1.89 (tt, J=8.4, 5.2 Hz, 1H), 0.95-0.85 (m, 2H), 0.62-0.51 (m, 2H).

### Intermediate NN134: Methyl 2-(2-bromo-5-fluoro-phenyl)-2-hydroxy-acetate

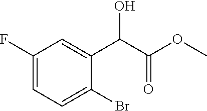

To a solution of (2-bromo-5-fluorophenyl)acetic acid methyl ester (490 mg, 1.88 mmol) in dry THE (9 mL) at −78° C. was added KHMDS (1 M in THF, 2.80 mL, 2.80 mmol). The reaction mixture was stirred at −78° C. for 30 min before the addition of 3-phenyl-2-(phenylsulfonyl)-1,2-oxaziridine (760 mg, 2.82 mmol). The reaction mixture was stirred at −78° C. for 1 h. The reaction mixture was warmed up to room temperature, quenched by addition of saturated aqueous NH₄Cl solution (15 mL) and extracted with EtOAc (3×20 mL). The combined organic extracts were washed with brine (30 mL), dried over Na₂SO₄, filtered, and concentrated under vacuum. The residue was purified by flash chromatography on silica gel (using a gradient of 0 to 50% EtOAc in Heptane as eluent) to afford the title compound (295 mg, yield: 60%) as a pale yellow oil. LC-MS m/z: no mass response; purity: 97%. ¹H NMR (400 MHz, DMSO-d₆) δ 7.67 (dd, J=8.9, 5.3 Hz, 1H), 7.31 (dd, J=8.9, 3.2 Hz, 1H), 7.18 (td, J=8.9, 3.2 Hz, 1H), 6.63 (d, J=5.8 Hz, 1H), 5.35 (d, J=5.8 Hz, 1H), 3.64 (s, 3H). ¹⁹F NMR (376 MHz, DMSO-d₆) δ−114.04 (td, J=8.9, 5.3 Hz).

### Intermediate NN135: Methyl 2-acetoxy-2-(2-bromo-5-fluoro-phenyl)acetate

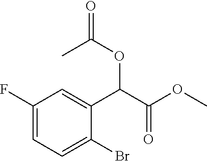 [SMILES: COC(=O)C(OC(C)=O)c1cc(F)ccc1Br]

To a solution of methyl 2-(2-bromo-5-fluoro-phenyl)-2-hydroxy-acetate NN134 (295 mg, 1.12 mmol) in dry DCM (5.6 mL) at room temperature were added Et₃N (0.80 mL, 6.00 mmol), DMAP (70.0 mg, 0.57 mmol) and Ac₂O (1.50 mL, 16.0 mmol). The reaction mixture was stirred at room temperature for 4 h. The reaction mixture was quenched by addition of saturated aqueous NaHCO₃ solution (10 mL) and extracted with EtOAc (3×15 mL). The combined organic extracts were washed with brine (20 mL), dried over Na₂SO₄, filtered, and concentrated under vacuum. The residue was purified by flash chromatography on silica gel (using a gradient of 0 to 30% EtOAc in Heptane as eluent) to afford the title compound (300 mg, yield: 82%) as a colorless oil. LC-MS m/z: no mass response; purity: 93%. ¹H NMR (400 MHz, DMSO-d₆) δ 7.78 (dd, J=8.6, 5.3 Hz, 1H), 7.36-7.26 (m, 2H), 6.30 (s, 1H), 3.71 (s, 3H), 2.17 (s, 3H).

### Intermediate NN213: ethyl 2-(3-bromo-5-fluoro-2-pyridyl)propanoate

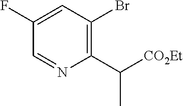

### Step 1: Synthesis of O1-tert-butyl O3-ethyl 2-(3-bromo-5-fluoro-2-pyridyl)propanedioate NN211

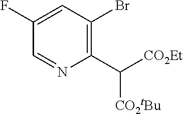 [SMILES: CCOC(=O)C(C(=O)OC(C)(C)C)c1ncc(F)cc1Br]

3-Bromo-2,5-difluoropyridine (163.3 g, 800 mmol) and dimethyl sulfoxide (3.2 L) were charged into a 5 L Process reactor. To the stirred solution was added tert-butyl ethyl malonate (300 mL, 1610 mmol) followed by Cs₂CO₃ (652 g, 2000 mmol) at rt. The resulting reaction mixture was stirred at 100° C. (internal temperature) for 21 hours before it was cooled down to rt, quenched with ice-cold water (1.7 L), and extracted with tert-butyl methyl ether (4×1.5 L). Combined organic extracts were washed with water (2×1.5 L), brine (2×1.2 L), dried over sodium sulfate, filtered and concentrated under vacuo to give the crude product (266 g) as a tan residue, which was then purified by flash chromatography (silica, gradient from 0 to 15% ethyl acetate in iso-hexane) to afford O1-tert-butyl O3-ethyl 2-(3-bromo-5-fluoro-2-pyridyl)propanedioate (120.6 g, 330 mmol, 42% Yield) as a white solid that spontaneously precipitated from chromatographed fractions. The precipitate was filtered off and the filtrates were concentrated and the resulting yellow residue (67 g) further purified by chromatography (silica, gradient from 0 to 10% ethyl acetate in hexane). The resulting pale yellow solid was slurried in ethyl acetate (20 mL) and iso-hexane (20 mL) and filtered off. The solid was washed with iso-hexane (2×20 mL) to afford O1-tert-butyl O3-ethyl 2-(3-bromo-5-fluoro-2-pyridyl)propanedioate (22.1 g, 61.0 mmol, 7.6% Yield). LC-MS (method B10) m/z: 306.0/308.0 (M-tBu+H), 262.0/264.0 (M-Boc+H); rt: 2.1 min, purity: 100%. ¹H NMR (300 MHz, CDCl₃) δ 8.41 (d, J=2.6 Hz, 1H), 7.66 (dd, J=7.5, 2.6 Hz, 1H), 5.10 (s, 1H), 4.29 (qd, J=7.2, 4.5 Hz, 2H), 1.49 (s, 9H), 1.29 (t, J=7.1 Hz, 3H). ¹⁹F NMR (282 MHz, CDCl₃) δ−125.48 (d, J=7.4 Hz). ¹³C NMR (101 MHz, CDCl₃) δ 166.98, 165.57, 157.80 (d, J=263.2 Hz), 149.25 (d, J=4.0 Hz), 136.19 (d, J=23.1 Hz), 127.43 (d, J=20.8 Hz), 121.16 (d, J=3.5 Hz), 82.90, 61.97, 60.01, 27.88, 14.06.

### Step 2: Synthesis of ethyl 2-(3-bromo-5-fluoro-2-pyridyl)acetate NN212

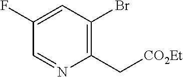 [SMILES: CCOC(=O)Cc1ncc(F)cc1Br]

To a 2 L Process Reactor were added dichloromethane (400 mL) and trifluoroacetic acid (300 mL). To the stirred solution was added a freshly prepared solution of O1-tert-butyl O3-ethyl 2-(3-bromo-5-fluoro-2-pyridyl)propanedioate NN211 (69.3 g, 191 mmol) in dichloromethane (400 mL) at rt. The reaction mixture was stirred at rt for 5 hours before it was quenched by pouring into two stirred beakers of ice-water (2×1.7 L). The stirred mixture was carefully neutralised with solid K₃PO₄ to pH 7. The mixtures were then partitioned and organic extracts combined. The separated aqueous layer was extracted with dichloromethane (2×1 L). The combined organic extracts were washed with saturated NaHCO₃ solution (1.5 L), dried over sodium sulfate and concentrated under vacuo to give ethyl 2-(3-bromo-5-fluoro-2-pyridyl)acetate (53.6 g, 200 mmol, 100% Yield) as a yellow oil. LC-MS (method B10) m/z: 262.0/264.0 [M+H]+; rt: 1.42 min, purity: 100%. ¹H NMR (300 MHz, CDCl₃) δ 8.39 (d, J=2.6 Hz, 1H), 7.66 (dd, J=7.5, 2.6 Hz, 1H), 4.20 (q, J=7.1 Hz, 2H), 4.02 (s, 2H), 1.27 (t, J=7.1 Hz, 3H). ¹⁹F NMR (282 MHz, CDCl₃) δ−126.48 (d, J=7.6 Hz). ¹³C NMR (101 MHz, CDCl₃) δ 169.43, 157.64 (d, J=262.2 Hz), 149.91, 136.18 (d, J=22.6 Hz), 127.39 (d, J=20.6 Hz), 121.11, 61.27, 43.00, 14.16.

### Step 3: Synthesis of ethyl 2-(3-bromo-5-fluoro-2-pyridyl)propanoate NN213

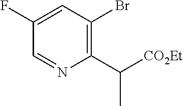

To a stirred solution of lithium diisopropylamide (2.0M in THF/heptane/ethylbenzene, 56 mL, 110 mmol) in anhydrous tetrahydrofuran (340 mL) under nitrogen at −74° C. was added a solution of ethyl 2-(3-bromo-5-fluoro-2-pyridyl)acetate NN212 (28.3 g, 100 mmol) in anhydrous tetrahydrofuran (100 mL) dropwise via a dropping funnel maintaining the internal temperature below −70° C. The resulting dark brown mixture was stirred for 30 minutes at −72° C. before iodomethane (9.6 mL, 150 mmol) was added dropwise. Stirring at −72° C. was continued for 30 minutes before the cooling bath was removed and the reaction mixture was allowed to warm up to rt. After 16 hours the reaction mixture was quenched with saturated NH₄Cl solution (450 mL) and extracted with ethyl acetate (2×450 mL). The combined organic extracts were washed with water (450 mL) and brine (300 mL), dried over sodium sulfate and concentrated under vacuo. Purification by chromatography (silica, gradient from 0 to 5% ethyl acetate in iso-hexane) afforded ethyl 2-(3-bromo-5-fluoro-2-pyridyl)propanoate (25.64 g, 92.86 mmol, 91% Yield) as a yellow oil. LC-MS (method B10) m/z: 276.0/278.0 [M+H]+; rt: 1.74 min, purity: 100%. ¹H NMR (300 MHz, CDCl₃) δ 8.40 (d, J=2.6 Hz, 1H), 7.64 (dd, J=7.6, 2.6 Hz, 1H), 4.33 (q, J=7.1 Hz, 1H), 4.16 (q, J=7.1 Hz, 2H), 1.53 (d, J=7.1 Hz, 3H), 1.20 (t, J=7.1 Hz, 3H). ¹⁹F NMR (282 MHz, CDCl₃) δ−126.94 (d, J=7.6 Hz).

### Intermediate NN287: Ethyl 2-(3-chloropyrazin-2-yl)propanoate

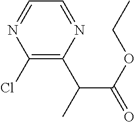

### Step 1: Synthesis of ethyl 2-(3-chloropyrazin-2-yl)acetate (NN288)

To a solution of 2,3-dichloropyrazine (2.50 g, 16.8 mmol) in THF (20 mL) was added a solution of LiHMDS in THF (1 M, 36.9 mL, 36.9 mmol) slowly at RT. The reaction mixture was stirred for 15 min at RT, then a solution of EtOAc (1.80 mL, 18.5 mmol) in toluene (15 mL) was added slowly at RT. The reaction mixture was stirred at RT for 18 hours, then diluted with EtOAc (50 mL) and washed with water (50 mL) and brine (2×50 mL). The organic phase was dried over Na₂SO₄, filtered and concentrated under vacuo. The crude was purified by column chromatography on silica gel (using a gradient of 0-100% EtOAc in isohexane as eluent) to afford the title compound as an orange oil (1.70 g, yield: 48%). LC-MS m/z [M+H]⁺: 201.2, 203.2; purity: 99%. ¹H NMR (400 MHz, DMSO-d₆) δ 8.64 (d, J=2.5 Hz, 1H), 8.48 (d, J=2.5 Hz, 1H), 4.13 (q, J=7.1 Hz, 2H), 4.05 (s, 2H), 1.19 (t, J=7.1 Hz, 3H).

### Step 2: Synthesis of ethyl 2-(3-chloropyrazin-2-yl)propanoate (NN287)

To a solution of ethyl 2-(3-chloropyrazin-2-yl)acetate NN288 (1.70 g, 8.47 mmol) in THF (20 mL) at 0° C. was added a solution of LiHMDS in THF (1.0 M, 10.2 mL, 10.2 mmol) slowly. The reaction mixture was stirred for 30 minutes at 0° C., then iodomethane (0.58 mL, 9.3 mmol) was added slowly and the reaction mixture was stirred at 0° C. for 2 hours. The reaction mixture was concentrated under vacuo and the crude was purified by column chromatography on silica gel (using a gradient of 0-100% EtOAc in isohexane as eluent) to afford the title compound as a light-yellow oil (1.61 g, yield: 82%). LC-MS m/z [M+H]⁺: 215.2, 217.2; purity: 93%. ¹H NMR (400 MHz, DMSO-d₆) δ 8.66 (d, J=2.5 Hz, 1H), 8.47 (d, J=2.5 Hz, 1H), 4.32 (q, J=7.1 Hz, 1H), 4.09 (q, J=7.1 Hz, 2H), 1.48 (d, J=7.1 Hz, 3H), 1.12 (t, J=7.1 Hz, 3H).

### Intermediate NN289: Methyl 2-(2-chloro-5-fluoro-3-pyridyl)acetate

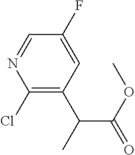

### Step 1: Synthesis of (2-chloro-5-fluoro-3-pyridyl)methanol (NN290)

To solution of 2-chloro-5-fluoro-pyridine-3-carbaldehyde (10.0 g, 62.7 mmol) in EtOH (50 mL) was added sodium borohydride (1.19 g, 31.3 mmol). The reaction mixture was stirred at RT for 2 hours, then carefully quenched with water (50 mL) and the suspension was diluted with brine (50 mL). The aqueous phase was extracted with EtOAc (3×100 mL) and the combined organic extracts were dried over Na₂SO₄, filtered and concentrated under vacuo to afford the title compound as a white solid (10.5 g, yield: 99%). LC-MS m/z [M+H]⁺: 162.2, 164.0; purity: 99%. ¹H NMR (400 MHz, DMSO-d₆) δ 8.35 (dd, J=3.0, 0.9 Hz, 1H), 7.80 (ddt, J=8.8, 3.0, 1.0 Hz, 1H), 5.73 (t, J=5.6 Hz, 1H), 4.53 (dt, J=5.5, 0.9 Hz, 2H).

### Step 2: Synthesis of 2-chloro-3-(chloromethyl)-5-fluoro-pyridine (NN291)

To a solution of (2-chloro-5-fluoro-3-pyridyl)methanol NN290 (10.5 g, 61.7 mmol) in DCM (100 mL) were added N,N-diisopropylethylamine (25.8 mL, 185 mmol) and methanesulfonyl chloride (6.2 mL, 80 mmol). The reaction mixture was stirred at RT for 18 hours then diluted with DCM (50 mL) and washed with an aqueous saturated solution of NaHCO₃ (100 mL) and brine (100 mL). The organic phase was filtered through a hydrophobic frit and concentrated under vacuo to afford the title compound as a yellow oil (10.2 g, yield: 88%). LC-MS m/z (no ionization); purity: 95%. ¹H NMR (400 MHz, CDCl₃) δ 8.24 (d, J=2.9 Hz, 1H), 7.65 (dd, J=8.0, 2.9 Hz, 1H), 4.66 (s, 2H).

### Step 3: Synthesis of 2-(2-chloro-5-fluoro-3-pyridyl)acetonitrile (NN292)

A solution of 2-chloro-3-(chloromethyl)-5-fluoro-pyridine NN291 (8.70 g, 48.3 mmol) and NaCN (2.60 g, 53.2 mmol) in DMF (100 mL) and water (30 mL) was stirred at 50° C. for 1 h then cooled to RT and diluted with an aqueous saturated solution of NaHCO₃ (150 mL). The aqueous phase was extracted with EtOAc (3×50 mL) and the combined organic extracts were washed with brine (2×100 mL), dried over Na₂SO₄, filtered and concentrated under vacuo. The residue was purified by column chromatography on silica gel (using a gradient of 0-60% EtOAc in isohexane) to afford the title compound as a yellow oil (6.80 g, yield: 57%). ¹H NMR (400 MHz, DMSO-d₆) δ 8.51 (d, J=2.9 Hz, 1H), 8.00 (dd, J=8.4, 3.0 Hz, 1H), 4.15 (s, 2H); purity 69%

### Step 4: Synthesis of methyl 2-(2-chloro-5-fluoro-3-pyridyl)acetate (NN289)

To a solution of 2-(2-chloro-5-fluoro-3-pyridyl)acetonitrile NN292 (69%, 6.80 g, 27.5 mmol) in MeOH (75 mL) was slowly added thionyl chloride (12 mL, 165 mmol). The reaction mixture was stirred at RT for 48 h, then concentrated under vacuo. The residue was partitioned between an aqueous saturated solution of NaHCO₃ (100 mL) and DCM (100 mL). The layers were separated and the aqueous phase was extracted with DCM (2×100 mL). The combined organic extracts were dried over Na₂SO₄, filtered and concentrated under vacuo. The product was purified by column chromatography on silica gel (using a gradient of 0-60% EtOAc in isohexane as eluent) to afford the title compound as a colourless oil (6.1 g, yield: 98%).LC-MS m/z [M+H]⁺: 204.2, 206.0; purity: 75%. ¹H NMR (400 MHz, DMSO-d₆) δ 8.42 (d, J=3.0 Hz, 1H), 7.95 (dd, J=8.8, 3.0 Hz, 1H), 3.88 (s, 2H), 3.66 (s, 3H).

### Intermediate NN293: Methyl 2-(2-chloro-5-fluoro-3-pyridyl)propanoate

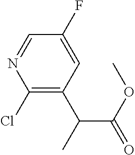

To a solution of methyl 2-(2-chloro-5-fluoro-3-pyridyl)acetate NN289 (36.2 mL, 13.3 mmol) in THF (33.8 mL) at 0° C. was added slowly a solution of LiHMDS in THF (1.0 M, 14.6 mL, 14.6 mmol). The reaction mixture was stirred at 0° C. for 30 min and then iodomethane (2.17 g, 15.3 mmol) was added. The reaction mixture was stirred at 0° C. for 2 h, then concentrated under vacuo. The crude was purified by column chromatography on silica gel (using a gradient of 0-60% EtOAc in isohexane) to afford the title compound as a yellow oil (2.05 g, yield: 63%). LC-MS m/z [M+H]⁺: 218.0, 220.1; purity: 88%. ¹H NMR (400 MHz, DMSO-d₆) δ 8.41 (dd, J=3.0, 1.1 Hz, 1H), 7.92 (dd, J=9.1, 3.0 Hz, 1H), 4.12 (q, J=7.2 Hz, 1H), 3.63 (s, 3H), 1.48 (d, J=7.2 Hz, 3H).

### Intermediate NN294: Ethyl 2-(2-bromo-4-fluoro-phenyl)propanoate

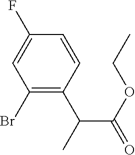

### Step 1: Synthesis of ethyl 2-(2-bromo-4-fluoro-phenyl)propanoate (NN295)

A solution of 2-(2-bromo-4-fluoro-phenyl)acetic acid (7.0 g, 30 mmol) and sulfuric acid (0.81 mL, 15.0 mmol) in EtOH (100 mL) was stirred at 80° C. for 2 h. The reaction mixture was cooled down to RT and concentrated under vacuo. The residue was partitioned between EtOAc (50 mL) and an aqueous saturated solution of NaHCO₃ (50 mL) and the layers separated. The organic phase was dried over Na₂SO₄, filtered and concentrated under vacuo to afford the title compound as a colourless oil (7.04 g, yield: 85%) as a colourless oil. ¹H NMR (400 MHz, DMSO-d₆) δ 7.58 (dd, J=8.6, 2.7 Hz, 1H), 7.47 (dd, J=8.5, 6.2 Hz, 1H), 7.25 (td, J=8.5, 2.7 Hz, 1H), 4.10 (q, J=7.1 Hz, 2H), 3.80 (s, 2H), 1.19 (t, J=7.1 Hz, 3H).

### Step 2: Synthesis of ethyl 2-(2-bromo-4-fluoro-phenyl)propanoate (NN294)

To a solution of ethyl 2-(2-bromo-4-fluoro-phenyl)acetate NN295 (7.00 g, 26.8 mmol) in THF (90 mL) was added LiHMDS in THF (1.00 mol/L, 34.9 mL, 34.9 mmol) dropwise at 0° C. The reaction mixture was stirred at 0° C. for 30 minutes and then iodomethane (1.84 mL, 29.5 mmol) was added slowly. The reaction mixture was stirred at 0° C. for 2 hours and then concentrated under reduced pressure. The crude was purified by column chromatography on silica gel (using a gradient of 0-100% EtOAc in isohexane) to afford the title compound as a yellow oil (6.0 g, yield: 77%). ¹H NMR (400 MHz, DMSO-d₆) δ 7.59 (dd, J=8.6, 2.7 Hz, 1H), 7.40 (dd, J=8.7, 6.1 Hz, 1H), 7.27 (td, J=8.5, 2.7 Hz, 1H), 4.08 (q, J=7.1 Hz, 3H), 1.40 (d, J=7.2 Hz, 3H), 1.13 (t, J=7.1 Hz, 3H).

### Intermediate NN296: Ethyl 2-(2-chloro-3-pyridyl)propanoate

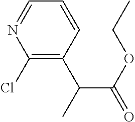

To a solution of ethyl 2-(2-chloro-3-pyridyl)acetate (CAS: 164464-60-2) (2.0 g, 10 mmol) in dry THE (30 mL) under nitrogen at −78° C. was added a solution of LiHMDS in THF (1.0 M, 11.5 mL, 11.5 mmol) dropwise. After stirring for 10 minutes, iodomethane (0.81 mL, 13 mmol) was added. After 1 h, the cooling bath was removed and the reaction mixture was allowed to warm up to RT. The reaction was stirred for 1 h then the reaction mixture was diluted with water (20 mL) and extracted with EtOAc (3×100 mL). The organic phase was dried over Na₂SO₄, filtered and concentrated under vacuum. The crude material was purified by column chromatography on silica gel (using a gradient of 0-100% EtOAc in heptane) to afford the title compound as a yellow liquid (1.88 g, 87%). LC-MS m/z [M+H]⁺: 214.2, 216.2; purity: 99%.

### Intermediate NN297: Methyl 2-(3-bromo-5-methoxy-2-pyridyl)propanoate

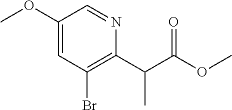

### Step 1: Synthesis of methyl 2-(3-bromo-5-methoxy-2-pyridyl)acetate (NN298)

To a solution of methyl 2-(3-bromo-5-fluoro-2-pyridyl)acetate (CAS: 1804408-40-9) (1.62 g, 5.78 mmol) in NMP (30 mL) was added MeONa (5.4 M in MeOH, 2.14 mL, 11.6 mmol) and the reaction mixture was heated at 120° C. for 30 min. The reaction mixture was cooled to RT and then water (100 mL) was added. The reaction mixture was extracted with EtOAc (3×100 mL). The combined organics were washed with brine (2×100 mL), dried over Na₂SO₄ and concentrated under vacuo to give the title product as a pale yellow oil (750 mg, 48%). LC-MS m/z [M+H]⁺: 260.2/262.0; purity: 100%. ¹H NMR (400 MHz, CDCl₃) δ 8.21 (d, J=2.6 Hz, 1H), 7.42 (d, J=2.6 Hz, 1H), 4.00 (s, 2H), 3.85 (s, 3H), 3.73 (s, 3H).

### Step 2: Synthesis of methyl 2-(3-bromo-5-methoxy-2-pyridyl)propanoate (NN297)

A solution of methyl 2-(3-bromo-5-methoxy-2-pyridyl)acetate NN298 (750 mg, 2.88 mmol) in THF (7 mL) was cooled to 0° C. LiHMDS (1 M in THF, 2.88 mL, 2.88 mmol) was added dropwise and the reaction mixture was stirred for 30 min. Iodomethane (0.21 mL, 3.46 mmol) was added and the reaction mixture stirred for 1 h at 0° C. The solvent was removed under vacuo and the residue was purified by column chromatography on silica gel (using a gradient of 0-25% EtOAc in isohexane as eluent) to afford methyl 2-(3-bromo-5-methoxy-2-pyridyl)propanoate (550 mg, yield: 70%) as a clear colorless oil. ¹H NMR (400 MHz, CDCl₃) δ 8.23 (d, J=2.6 Hz, 1H), 7.39 (d, J=2.6 Hz, 1H), 4.32 (q, J=7.1 Hz, 1H), 3.84 (s, 3H), 3.69 (s, 3H), 1.52 (d, J=7.1 Hz, 3H).

### Intermediate NN299: Ethyl 2-(3-bromo-2-pyridyl)-3-(2-trimethylsilylethoxy)propanoate

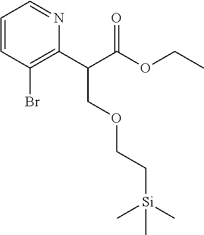 [SMILES: CCOC(=O)C(COCC[Si](C)(C)C)c1ncccc1Br]

To a solution of ethyl 2-(3-bromo-2-pyridyl)acetate (CAS: 197376-41-3) (8.7 g, 34 mmol) in THF (100 mL), cooled at −60° C., was added LDA (2M solution in THF-ethyl benzene-heptane, 18.6 mL, 37.2 mmol) over 20 minutes. Upon completion of the addition, the reaction mixture was stirred at −60° C. for a further 20 minutes before a dropwise addition of 2-(trimethylsilyl)ethoxymethyl chloride (17.8 mL, 102 mmol). The reaction mixture was then allowed to warm to 0° C. over 1 h. The reaction mixture was poured onto water (120 mL) and the crude product was extracted with EtOAc (2×100 mL). The combined organic extracts were dried over Na₂SO₄, filtered and concentrated under vacuo. The residue was purified by column chromatography on silica gel (using a gradient of 0-100% MTBE in isohexane as eluent) to afford the title compound as a colorless oil (12.1 g, yield: 90%). LC-MS m/z [M+H]⁺: 374.1, 376.0; purity: 100%. ¹H NMR (400 MHz, DMSO-d₆) δ 8.52 (dd, J=4.6, 1.5 Hz, 1H), 8.09 (dd, J=8.1, 1.5 Hz, 1H), 7.27 (dd, J=8.1, 4.6 Hz, 1H), 4.51 (t, J=7.1 Hz, 1H), 4.05 (qd, J=7.1, 2.5 Hz, 2H), 3.97 (dd, J=9.4, 6.9 Hz, 1H), 3.84 (dd, J=9.4, 7.3 Hz, 1H), 3.57-3.38 (m, 2H), 1.10 (t, J=7.1 Hz, 3H), 0.82-0.73 (m, 2H), −0.09 (s, 9H).

### Intermediate NN300: Ethyl 2-acetoxy-2-(3-bromo-5-fluoro-2-pyridyl)acetate

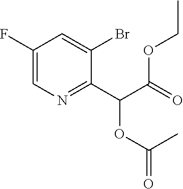 [SMILES: CCOC(=O)C(OC(C)=O)c1ncc(F)cc1Br]

### Step 1: Synthesis of ethyl 2-bromo-2-(3-bromo-5-fluoro-2-pyridyl)acetate (NN301)

To a solution of ethyl 2-(3-bromo-5-fluoro-2-pyridyl)acetate NN212 (1 g, 3.82 mmol) in THF (45 mL) was added LiHMDS (1 M in THF, 4.58 mL, 4.58 mmol) at 0° C. The reaction mixture was stirred at 0° C. for 45 min and then NBS (1.02 g, 5.72 mmol) was added. The reaction mixture was allowed to warm to RT and stirred for 18 h. The reaction mixture was quenched with water (100 mL) and extracted with EtOAc (3×100 mL). The combined organic extracts were dried over Na₂SO₄, filtered and concentrated under vacuo. The residue was purified by column chromatography on silica gel (using a gradient of 0-25% EtOAc in isohexane as eluent) to give the title compound as a colorless oil (758 mg, yield: 49%). LC-MS m/z [M+H]⁺: 339.9, 341.9, 343.9; purity: 76.6%. ¹H NMR (400 MHz, CDCl₃) δ 8.43 (d, J=2.6 Hz, 1H), 7.69 (dd, J=7.4, 2.5 Hz, 1H), 5.95 (s, 1H), 4.30 (q, J=7.1 Hz, 2H), 1.28 (t, J=7.2 Hz, 3H).

### Step 2: Synthesis of ethyl 2-acetoxy-2-(3-bromo-5-fluoro-2-pyridyl)acetate (NN300)

To a solution of ethyl 2-bromo-2-(3-bromo-5-fluoro-2-pyridyl)acetate NN301 (85% purity, 708 mg, 1.76 mmol) in DMF (35 mL) was added CH₃COOK (866 mg, 8.82 mmol) and the reaction mixture was heated at 80° C. for 18 h. The reaction mixture was cooled to RT. The reaction mixture was then diluted with EtOAc (150 mL) and washed with brine (3×100 mL). The organic layer was dried over Na₂SO₄ and the solvent was removed under vacuo. The residue was purified by column chromatography on silica gel (using a gradient of 0-25% EtOAc in isohexane as eluent) to afford the title compound as a colorless oil (496 mg, yield: 83%). LC-MS m/z [M+H]⁺: 320.0, 322.0; purity: 95%. ¹H NMR (400 MHz, CDCl₃) δ 8.44 (d, J=2.6 Hz, 1H), 7.71 (dd, J=7.5, 2.6 Hz, 1H), 6.55 (s, 1H), 4.28 (q, J=7.1 Hz, 2H), 2.20 (s, 3H), 1.25 (t, J=7.1 Hz, 3H).

### Intermediate NN302: Methyl 2-(2-bromo-3-thienyl)propanoate

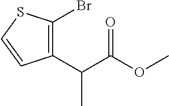

### Step 1: Synthesis of methyl 2-(2-bromo-3-thienyl)acetate (NN303)

To a solution of methyl 2-(3-thienyl)acetate (1.50 g, 9.60 mmol) in THF (15 mL) was added NBS (1.73 g, 9.70 mmol). The reaction mixture was refluxed for 60 hours. The volatiles were removed under reduced pressure and the crude was purified by column chromatography on silica gel (using a gradient of 0-20% EtOAc in isohexane as eluent) to afford the title compound as a colourless oil (1.97 g, yield: 70%). ¹H NMR (400 MHz, CDCl₃) δ 7.24 (d, J=5.7 Hz, 1H), 6.93 (d, J=5.6 Hz, 1H), 3.72 (s, 3H), 3.64 (s, 2H); purity: 80%.

### Step 2: Synthesis of methyl 2-(2-bromo-3-thienyl)propanoate (NN302)

To a solution of methyl 2-(2-bromo-3-thienyl)acetate NN303 (80% purity, 500 mg, 1.70 mmol) in THE (10 mL) cooled down to 0° C. was added slowly LiHMDS 1 M in THF (2.04 mL, 2.04 mmol) and the reaction mixture was stirred at 0° C. for 1 h. Iodomethane (362 mg, 2.55 mmol) was added, and the reaction mixture stirred at 0° C. for 1 h then at RT for a further 20 h. The reaction mixture was diluted with saturated aqueous NH₄Cl (5 mL) then extracted with EtOAc (3×10 mL). The combined organic extracts were washed with brine (10 mL) then dried over Na₂SO₄, filtered and concentrated under reduced pressure. The crude was purified by column chromatography on silica gel (using a gradient of 0-15% EtOAc in isohexane as eluent) to afford the title compound as a colourless oil (320 mg, yield: 64%). ¹H NMR (400 MHz, DMSO-d₆) δ 7.59 (d, J=5.7 Hz, 1H), 6.97 (d, J=5.7 Hz, 1H), 3.86 (q, J=7.1 Hz, 1H), 3.61 (s, 3H), 1.37 (d, J=7.2 Hz, 3H); purity: 85%.

### Intermediate NN304: Ethyl 2-[5-fluoro-3-(4,4,5,5-tetramethyl-1,3,2-dioxaborolan-2-yl)-2-pyridyl]propanoate

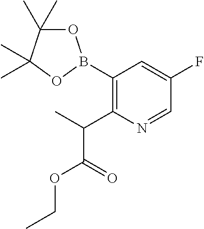 [SMILES: CCOC(=O)C(C)c1ncc(F)cc1B1OC(C)(C)C(C)(C)O1]

To a nitrogen sparged suspension of ethyl 2-(3-bromo-5-fluoro-2-pyridyl)propanoate NN213 (86% purity, 1.50 g, 4.67 mmol), 4,4,5,5-tetramethyl-2-(4,4,5,5-tetramethyl-1,3,2-dioxaborolan-2-yl)-1,3,2-dioxaborolane (1.4 g, 5.61 mmol) and CH₃COOK (1.8 g, 18.7 mmol) in 1,4-dioxane (13 mL) was added [1,1′-bis(diphenylphosphino)ferrocene]dichloropalladium(II) DCM complex (172 mg, 0.21 mmol) and the reaction mixture was sparged with nitrogen for a further 10 min. The suspension was heated at reflux for 4 h. The reaction mixture was allowed to cool to room temperature then filtered over celite and washed through with EtOAc (100 mL). The filtrate was concentrated under reduced pressure then purified by column chromatography on silica gel (using a gradient of 0-100% EtOAc in isohexane as eluent) to afford the title compound as a clear colourless oil which partially crystallised upon standing (950 mg, yield: 35%). LC-MS (Method A9) m/z [M+H]⁺: 324.4; rt: 1.27 min, purity: 55%. ¹H NMR (400 MHz, CDCl₃) δ 8.42 (d, J=3.1 Hz, 1H), 7.77 (dd, J=8.8, 3.1 Hz, 1H), 4.67 (q, J=7.0 Hz, 1H), 4.17-4.09 (m, 2H), 1.50 (d, J=7.1 Hz, 3H), 1.35 (s, 12H), 1.18 (t, J=7.2 Hz, 3H); purity: 55%.

### Intermediate NN305: Ethyl 2-(5-bromo-2-methyl-pyrimidin-4-yl)propanoate

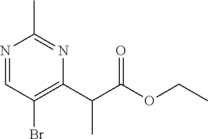

### Step 1: Synthesis of O1-tert-butyl O3-ethyl 2-(5-bromo-2-methyl-pyrimidin-4-yl)propanedioate (NN306)

NaH (60%, 37 mg, 0.92 mmol) was added portionwise to a stirring solution of 0-3-tert-butyl 0-1-ethyl propanedioate (10 mL, 52 mmol) in dry THF (57 mL) at 0 C under nitrogen. The reaction mixture was warmed to RT and stirred for 10 minutes under nitrogen and 5-bromo-4-chloro-2-methyl-pyrimidine (5.7 g, 27.5 mmol) was then added. The reaction mixture was heated at reflux for 4 h then cooled to RT. A saturated aq. solution of NH₄Cl (100 mL) was added and was extracted with EtOAc (3×60 mL). The combined organic extracts were washed with brine (10 mL), dried over Na₂SO₄, filtered and concentrated under reduced pressure. The residue was purified by column chromatography on silica gel (using a gradient of 0-50% MTBE in isohexane as eluant) to afford the title compound as a pale yellow oil (8.48 g, yield: 83%). LC-MS m/z [M+H]⁺: 359.1, 361.1; purity: 97%. ¹H NMR (400 MHz, CDCl₃) δ 8.67 (s, 1H), 4.98 (s, 1H), 4.39-4.24 (m, 2H), 2.67 (s, 3H), 1.50 (s, 9H), 1.30 (t, J=7.1 Hz, 3H).

### Step 2: Synthesis of ethyl 2-(5-bromo-2-methyl-pyrimidin-4-yl)acetate (NN307)

To a solution of O1-tert-butyl O3-ethyl 2-(5-bromo-2-methyl-pyrimidin-4-yl)propanedioate NN306 (8.48 g, 23.6 mmol) in DCM (25.0 mL) was added TFA (15.0 mL, 202 mmol). The solution was stirred at RT overnight then concentrated under vacuum. It was then azeotroped with toluene (150 mL). The residue was partitioned between a saturated aq. solution of NaHCO₃ (100 mL) and EtOAc (100 mL). The phases were extracted and separated. The aqueous layer was extracted with EtOAc (100 mL). The combined extracts were washed with brine (100 mL), dried over Na₂SO₄, filtered and concentrated to give the title compound as a yellow oil (6.35 g, yield: 93%). LC-MS m/z [M+H]⁺: 259.2, 261.2; purity: 95%. ¹H NMR (400 MHz, CDCl₃) δ 8.67 (s, 1H), 4.21 (q, J=7.1 Hz, 2H), 3.94 (s, 2H), 2.68 (s, 3H), 1.27 (t, J=7.1 Hz, 3H).

### Step 3: Synthesis of ethyl 2-(5-bromo-2-methyl-pyrimidin-4-yl)propanoate (NN305)

To a solution of ethyl 2-(5-bromo-2-methyl-pyrimidin-4-yl)acetate NN307 (6.35 g, 22.1 mmol) in THE (79 mL) at 0° C. was added LiHMDS 1 M in THF (26.5 mL, 26.5 mmol) dropwise. The resulting mixture was stirred at 0° C. for 1 h then iodomethane (4.7 g, 33.1 mmol) was added dropwise. The reaction mixture was stirred at 0° C. for 1 h then at RT for 3 days. The reaction mixture was quenched with saturated NH₄C1 solution (100 mL) and was extracted with EtOAc (3×100 mL). The combined organic extracts were washed with brine (150 mL), dried over Na₂SO₄, filtered and concentrated under reduced pressure. The residue was purified by column chromatography on silica gel (using a gradient of 0-50% MTBE in isohexane as eluant) to afford the title compound as a pale yellow oil (5.49 g, yield: 87%). LC-MS m/z [M+H]⁺: 273.0, 275.0; purity: 93%. ¹H NMR (400 MHz, CDCl₃) δ 8.64 (s, 1H), 4.25-4.10 (m, 3H), 2.66 (s, 3H), 1.53 (d, J=7.1 Hz, 3H), 1.21 (t, J=7.1 Hz, 3H)

### Intermediate NN308: -Ethyl 2-(3-chloropyridazin-4-yl)propanoate

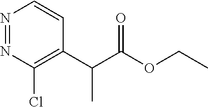

### Step 1: Synthesis of O1-tert-butyl O3-ethyl 2-(3-chloropyridazin-4-yl)propanedioate (NN309)

To a solution of 3,4-dichloropyridazine (90% purity, 3.00 g, 18.1 mmol) and O3-tert-butyl O1-ethyl propanedioate (3.4 g, 18 mmol) in DMSO (100 mL) was added Cs₂CO₃ (12 g, 36 mmol). After being heated with stirring at 100° C. for 18 h, the reaction mixture was cooled down to RT, diluted with DCM (200 mL), quenched with water (100 mL), and washed with brine (100 mL). The organic layer was dried over Na₂SO₄ and concentrated under vacuo and purified by column chromatography on silica gel (using a gradient of 0-100% EtOAc in isohexane as eluant) to afford the title compound as a yellow oil (3.1 g, yield: 55%). LC-MS m/z [M+H]⁺: 301.1, 303.1; purity: 97%

### Step 2: Synthesis of ethyl 2-(3-chloropyridazin-4-yl)acetate (NN310)

O1-tert-butyl O3-ethyl 2-(3-chloropyridazin-4-yl)propanedioate NN309 (1.0 g, 3.2 mmol) was dissolved in DCM (40.0 mL) and TFA (8.4 mL, 113 mmol) was slowly added. The reaction mixture was stirred at RT for 18 h. The reaction mixture was concentrated under vacuo and the residue partitioned between EtOAc (100 mL) and sat. NaHCO₃ (50 mL). The organic extract was dried over Na₂SO₄, filtered and concentrated under vacuo. The residue was purified by column chromatography on silica gel (using a gradient of 0-100% EtOAc in isohexane as eluant) to afford the title compound as a yellow oil (608 mg, yield: 89%). LC-MS m/z [M+H]⁺: 201.1, 203.1; purity: 100%. ¹H NMR (400 MHz, CDCl₃) δ 9.08 (d, J=4.9 Hz, 1H), 7.49 (d, J=4.9 Hz, 1H), 4.21 (q, J=7.1 Hz, 2H), 3.78 (s, 2H), 1.27 (t, J=7.1 Hz, 3H)

### Step 3: Synthesis of ethyl 2-(3-chloropyridazin-4-yl)propanoate (NN308)

Ethyl 2-(3-chloropyridazin-4-yl)acetate NN310 (500 mg, 2.37 mmol) was dissolved in dry THF (25.0 mL) under nitrogen. The reaction mixture was cooled to 0° C. and LiHMDS 1 M in THF (1.0 M, 2.7 mL, 2.7 mmol) was added dropwise. After stirring for 15 minutes, iodomethane (437 mg, 3.1 mmol) was added. After 1 h, the cooling bath was removed and the reaction mixture was allowed to warm to R. The reaction was stirred for 1 h then it was diluted with water (30 mL) and extracted with EtOAc (3×30 mL). The organic phase was dried over Na₂SO₄, filtered and concentrated under reduced pressure. The residue was purified by column chromatography on silica gel (using a gradient of 0-100% EtOAc in heptane as eluant) to afford the title compound as a yellow oil (333 mg, yield: 62%). ¹H NMR (400 MHz, CDCl₃) δ 9.09 (d, J=5.0 Hz, 1H), 7.50 (dd, J=5.2, 0.8 Hz, 1H), 4.18 (qd, J=7.1, 2.9 Hz, 2H), 4.13-4.05 (m, 1H), 1.56 (d, J=7.2 Hz, 3H), 1.24 (t, J=7.0 Hz, 3H).

### Intermediate NN311: Ethyl 2-(4-chloropyridazin-3-yl)propanoate

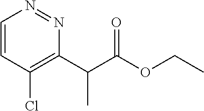

### Step 1: Synthesis of tert-butyl 2-(4-benzyloxypyridazin-3-yl)propanoate (NN312)

A solution of 4-benzyloxy-3-chloro-pyridazine N34_2 (2.79 g, 12.4 mmol) in dry THF (30 mL) was degassed with nitrogen for 5 minutes. XPhos Pd(crotyl)Cl (417 mg, 0.62 mmol) was added and then a solution of bromo-(2-tert-butoxy-1-methyl-2-oxo-ethyl)zinc (0.4 M in THF) (46.5 mL, 18.6 mmol) in THF was added slowly. The reaction mixture was allowed to stir at 45° C. for 30 minutes. The reaction mixture was cooled in ice/water and quenched with sat. NH₄Cl (150 mL), and extracted with EtOAc (2×100 mL). The organics were combined and dried over MgSO₄, filtered and concentrated under reduced pressure and the crude residue was purified by column chromatography on silica gel (using a gradient of 0-100% EtOAc in isohexane as eluant) to afford the title compound as a pale yellow foam (4.1 g, yield: 98%). LC-MS m/z [M+H]⁺: 315.2; purity: 98%. ¹H NMR (400 MHz, DMSO-d₆) δ 8.96 (d, J=5.9 Hz, 1H), 7.49-7.36 (m, 5H), 7.31 (d, J=6.0 Hz, 1H), 5.32-5.22 (m, 2H), 4.13 (q, J=7.2 Hz, 1H), 1.48 (d, J=7.2 Hz, 3H), 1.25 (s, 9H).

### Step 2: Synthesis of ethyl 2-(4-hydroxypyridazin-3-yl)propanoate (NN313)

A suspension of tert-butyl 2-(4-benzyloxypyridazin-3-yl)propanoate NN312 (4.1 g, 11.8 mmol) and 10% Pd/C (628 mg, 0.59 mmol) in EtOH (70 mL) was stirred at RT under 5 bar of hydrogen atmosphere for 18 h. The reaction mixture was filtered through a pad of celite and the filter cake was rinsed with EtOH (50 mL). The filtrate was concentrated under vacuo and the residue was dissolved in EtOH (80 mL). HCl (4 N in 1,4-dioxane) (2.95 mL, 11.8 mmol) was added and the resulting solution was stirred at 90° C. for 10 h. The reaction mixture was cooled down to RT and concentrated under vacuo to afford the title compound as a yellow foam (2.1 g, yield: 83%). LC-MS m/z [M+H]⁺: 197.1; purity: 96%. ¹H NMR (400 MHz, DMSO-d₆) δ 8.29 (d, J=7.4 Hz, 1H), 6.38 (d, J=7.3 Hz, 1H), 4.03 (qd, J=7.1, 0.8 Hz, 2H), 3.91 (q, J=7.2 Hz, 1H), 1.33 (d, J=7.2 Hz, 3H), 1.12 (t, J=7.1 Hz, 3H). The OH peak was not observed.

### Step 3: Synthesis of ethyl 2-(4-chloropyridazin-3-yl)propanoate (NN311)

To a solution of ethyl 2-(4-hydroxypyridazin-3-yl)propanoate NN313 (1.8 g, 8.3 mmol) in MeCN (12 mL), was added phosphorus oxychloride (1.9 mL, 21 mmol) at RT and the reaction was heated at 80° C. for 1 h. The reaction mixture was allowed to cool to RT, poured onto a solution of sat. NaHCO₃ solution (250 mL) and the crude product was extracted with EtOAc (3×100 mL). The organics were combined, dried over Na₂SO₄, filtered and concentrated under vacuo. The crude residue was purified by column chromatography on silica gel (using a gradient of 0-100% EtOAc in isohexane as eluant) to afford the title compound as a pale yellow oil (1.8 g, yield: 94%). LC-MS m/z [M+H]⁺: 215.1, 217.1; purity: 93%. ¹H NMR (400 MHz, DMSO-d₆) δ 9.14 (d, J=5.5 Hz, 1H), 7.97 (d, J=5.5 Hz, 1H), 4.45 (q, J=7.2 Hz, 1H), 4.10 (q, J=7.1 Hz, 2H), 1.55 (d, J=7.1 Hz, 3H), 1.12 (t, J=7.1 Hz, 3H).

### Intermediate NN314: Ethyl 2-(5-chloropyridazin-4-yl)propanoate

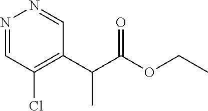

### Step 1: Synthesis of O1-tert-butyl O3-ethyl 2-(5-chloropyridazin-4-yl)propanedioate (NN315)

To a solution of 4,5-dichloropyridazine (500 mg, 3.4 mmol) and tert-butyl ethyl malonate (632 mg, 3.4 mmol) in DMSO (20 mL) was added Cs₂CO₃ (2.2 g, 6.7 mmol) and the reaction mixture was heated with stirring at 100° C. for 4 h. The reaction mixture was allowed to cooldown to RT and quenched with water (100 mL), then extracted with EtOAc (2×100 mL). The combined organics were washed with brine (2×100 mL). The organic layer was dried over Na₂SO₄ and concentrated under vacuo. The residue was purified by column chromatography on silica gel (using a gradient of 0-25% EtOAc in isohexane as eluant) to afford the title compound as an orange oil (662 mg, yield: 62%). LC-MS m/z [M+H]⁺: 301.2, 303.2; purity: 100%.

### Step 2: Synthesis of ethyl 2-(5-chloropyridazin-4-yl)acetate (NN316)

O1-tert-butyl O3-ethyl 2-(5-chloropyridazin-4-yl)propanedioate NN315 (662 mg, 2.1 mmol) was dissolved in DCM (27 mL) and TFA (5.4 mL, 73 mmol) was slowly added. The reaction mixture was stirred at RT for 18 h. A sat. aq. solution of NaHCO₃ was added and the reaction mixture stirred until the evolution of gas had ceased. The aqueous phase was extracted with DCM (3×100 mL). The organic phase was dried over Na₂SO₄, filtered and concentrated under vacuo. EtOAc (50 mL) was added resulting in the precipitation of a black solid. The flask was stoppered and left to stand overnight, after which time the solid has settled. The liquors were pipetted off and concentrated to afford the title compound as dark brown gum (414 mg, yield: 95%). ¹H NMR (400 MHz, CDCl₃) δ 9.17 (d, J=0.8 Hz, 1H), 9.08 (s, 1H), 4.21 (q, J=7.1 Hz, 2H), 3.80 (s, 2H), 1.27 (t, J=7.1 Hz, 3H).

### Step 3: Synthesis of ethyl 2-(5-chloropyridazin-4-yl)propanoate (NN314)

To a stirred solution of ethyl 2-(5-chloropyridazin-4-yl)acetate NN316 (414 mg, 1.98 mmol) in tetrahydrofuran (10 mL) was added LiHMDS 1 M in THF (1.0 M, 1.98 mL, 1.98 mmol) dropwise at 0 C. After 30 min, iodomethane (0.15 mL, 2.37 mmol) was added and the resulting reaction mixture was allowed to reach room temperature and stirred for further 1 h. The solvent was concentrated under vacuo and the residue was purified by column chromatography on silica gel (using a gradient of 0-70% EtOAc in isohexane as eluant) to afford the title compound as an orange/brown oil (97 mg, yield: 22%). LC-MS m/z [M+H]⁺: 215.2, 217.2; purity: 95%. ¹H NMR (400 MHz, CDCl₃) δ 9.13 (d, J=0.8 Hz, 1H), 9.10 (s, 1H), 4.19 (q, J=7.1 Hz, 2H), 4.13 (q, J=7.3 Hz, 1H), 1.59 (d, J=7.3 Hz, 3H), 1.23 (t, J=7.1 Hz, 3H).

### Intermediate NN439: ethyl 2-(4-chloropyrimidin-5-yl)propanoate

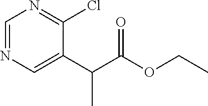

To a solution of ethyl 2-(4-chloropyrimidin-5-yl)acetate (CAS 6214-47-7) (250 mg, 1.25 mmol) in THE (5 mL) at 0° C. was added LiHMDS (1 M in THF, 1.48 mL, 1.48 mmol) via syringe slowly. The reaction mixture was stirred at 0° C. for 30 minutes and Mel (230 mg, 1.62 mmol) was added. The reaction mixture was stirred at 0° C. for 2 h. The reaction mixture was concentrated under reduced pressure to afford the title compound as a brown oil (265 mg, yield: 91%). LC-MS m/z [M+H]⁺: 215.1, 217.2; purity: 92%.

The following intermediate lactams have been prepared following similar procedures as the ones used for the preparation of related lactames, such as Intermediates N1-N4, N6, N8-23, N25-N37, N39-N41, N43-N45, N53-N58, N59a, N59b, N60-N61, N63-N64, N65_1, N66, N68_4, N69_2, N71_2. N72 6. N73_3, N75-N78, N79-N128 starting either from the corresponding bromo or boronate of the amino-aryl or heteroaryl and from the corresponding boronate or bromo aryl- or heteroaryl-esters, respectively (specific intermediates referenced in the table below).

| # | Structure | Chemical name | Method and Characterization |
|---|---|---|---|
| NN136 | 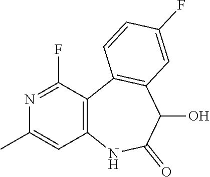 [SMILES: Cc1cc2c(c(F)n1)-c1ccc(F)cc1C(O)C(=O)N2] | 1,9-difluoro-7- hydroxy-3-methyl- 5,7- dihydropyrido[4,3- d][3]benzazepin-6- one | Suzuki reaction using methyl 2-acetoxy-2- (2-bromo-5-fluoro-phenyl)acetate (Intermediate NN135), 2-fluoro-6-methyl- 3-(4,4,5,5-tetramethyl-1,3,2-dioxaborolan- 2-yl)pyridin-4-amine (Intermediate N16_1), K₃PO₄, Pd₂dba₃/SPhos heated in toluene at 100° C. followed by cyclization by reaction with LiHMDS in THF at 0° C. (44% yield). LC-MS m/z: [M + H]⁺: 276.9; purity: 89%. ¹H NMR (400 MHz, DMSO-d₆) δ 10.88 (s, 1H), 7.69 (dt, J = 8.6, 5.1 Hz, |
| | | | 1H), 7.41 (dd, J = 10.0, 2.9 Hz, 1H), 7.28 |
| | | | (td, J = 8.6, 2.9 Hz, 1H), 6.97 (s, 1H), 6.12 |
| | | | (d, J = 5.9 Hz, 1H), 4.88 (d, J = 5.9 Hz, 1H), |
| | | | 2.44 (s, 3H). 19F NMR (376 MHz, DMSO- |
| | | | d₆) δ −69.85 (d, J = 5.1 Hz), −111.97-−112.09 |
| | | | (m). |
| NN142 | 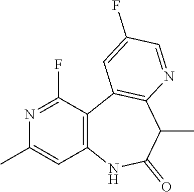 | 3,14-difluoro-5,10- dimethyl-4,8,12- triazatricyclo[9.4.0.0^{2,7}] pentadeca- 1(11),2(7),3,5,12,14- hexaen-9-one | Suzuki reaction using ethyl 2-(3-bromo-5- fluoro-2-pyridyl)propanoate (Intermediate NN213), 2-fluoro-6-methyl-3-(4,4,5,5- tetramethyl-1,3,2-dioxaborolan-2- yl)pyridin-4-amine (Intermediate N16_1), K₃PO₄, tris(dibenzylideneacetone)dipalladium(0)/ 2-dicyclohexylphosphino-2,6- dimethoxybiphenyl heated in toluene at 100° C. followed by cyclization with LiHMDS in THF at 0° C. (32% yield). LC- MS m/z [M + H]⁺: 276.0; purity: 100%. ¹H |
| | | | NMR (400 MHz, DMSO-d₆) δ 10.87 (bs., |
| | | | 1H), 8.69 (d, J = 2.8 Hz, 1H), 8.08 (ddd, J = |
| | | | 9.7, 4.0, 2.8 Hz, 1H), 7.01 (s, 1H), 3.64 |
| | | | (q, J = 6.6 Hz, 1H), 2.46 (s, 3H), 1.48 (d, J = |
| | | | 6.6 Hz, 3H). 19F NMR (376 MHz, |
| | | | DMSO-d₆) δ −70.19 (d, J = 4.0 Hz), −130.44 |
| | | | (d, J = 9.7 Hz). |
| NN162 | 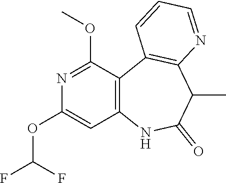 [SMILES: COc1nc(OC(F)F)cc2c1-c1cccnc1C(C)C(=O)N2] | 5- (difluoromethoxy)- 3-methoxy-10- methyl-4,8,12- triazatricyclo[9.4.0.0^{2,7}] pentadeca- 1(11),2(7),3,5,12,14- hexaen-9-one | Suzuki reaction using ethyl 2-[3-(4,4,5,5- tetramethyl-1,3,2-dioxaborolan-2-yl)-2- pyridyl]propanoate (Intermediate N31_1), 3-bromo-6-(difluoromethoxy)-2-fluoro- pyridin-4-amine (Intermediate NN161), K₂CO₃, Pd[(amphos)Cl]₂ heated in dioxane/water at 100° C. followed by cyclization with K₂CO₃ in MeOH at RT (48% yield). LC-MS (Method B2) m/z: [M + H]⁺: 322.0; rt: 3.89 & 3.98 min (isomers); purity: 96%. ¹H NMR (400 MHz, DMSO-d₆) δ 10.62 (s, 1H), 8.61-8.56 (m, 1H), 8.14- |
| | | | 8.09 (m, 1H), 7.82 (t, J = 72.8 Hz, 1H), 7.42- |
| | | | 7.36 (m, 1H), 6.44 (s, 1H), 3.93 (s, 3H), |
| | | | 3.55 (q, J = 6.8 Hz, 1H), 1.46 (d, J = 6.7 |
| | | | Hz, 3H). 19F NMR (376 MHz, DMSO-d₆) δ |
| | | | −86.88 (dd, J = 72.8, 5.7 Hz). |
| NN166 | 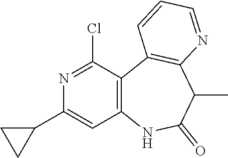 [SMILES: CC1C(=O)Nc2cc(C3CC3)nc(Cl)c2-c2cccnc21] | 3-chloro-5- cyclopropyl-10- methyl-4,8,12- triazatricyclo[9.4.0.0^{2,7}] pentadeca- 1(11),2(7),3,5,12,1 4-hexaen-9-one | Suzuki reaction using 3-bromo-2-chloro-6- cyclopropyl-pyridin-4-amine (Intermediate NN165), ethyl 2-[3-(4,4,5,5-tetramethyl- 1,3,2-dioxaborolan-2-yl)-2- pyridyl]propanoate (Intermediate N31_1), K₃PO₄, Pd₂dba₃/SPhos heated in toluene at 80° C. followed by cyclization with LiHMDS in toluene at RT (44% yield). LC- MS m/z: [M + H]⁺: 300.1/302.1; purity: 95%. LC-MS (Method B2) m/z: |
| | | | [M + H]⁺: 300.1/302.1; rt: 3.97 min; purity: |
| | | | 95%. |
| NN167 | 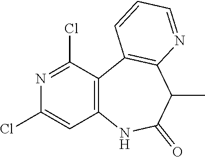 [SMILES: CC1C(=O)Nc2cc(Cl)nc(Cl)c2-c2cccnc21] | 3,5-dichloro-10- methyl-4,8,12- triazatricyclo[9.4.0.0^{2,7}] pentadeca- 1(11),2(7),3,5,12,14- hexaen-9-one | Suzuki reaction using 3-bromo-2,6- dichloropyridin-4-amine (500.0 mg, 1.9636 mmol), ethyl 2-[3-(4,4,5,5- tetramethyl-1,3,2-dioxaborolan-2-yl)-2- pyridyl]propanoate (Intermediate N31_1), K₂CO₃, bis(di-tert-butyl(4- dimethylaminophenyl)phosphine)dichloro- palladium(II) heated in dioxane/water at 100° C. followed by cyclization with LiHMDS in toluene at RT (15% yield). LC- |
| | | | MS m/z [M + H]⁺: 294.0/296.1/298.0; purity: |
| | | | 91%. ¹H NMR (400 MHz, DMSO-d₆) δ |
| | | | 10.84 (s, 1H), 8.70-8.66 (s, 1H), 8.25- |
| | | | 8.20 (m, 1H), 7.50-7.44 (m, 1H), 7.30- |
| | | | 7.27 (m, 1H), 3.75 (q, J = 6.5 Hz, 1H), 1.48 |
| | | | (d, J = 6.5 Hz, 3H). |
| NN168 | 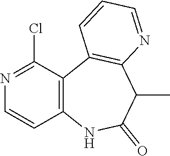 [SMILES: CC1C(=O)Nc2ccnc(Cl)c2-c2cccnc21] | 3-chloro-10- methyl-4,8,12- triazatricyclo[9.4.0.0^{2,7}] pentadeca- 1(11),2(7),3,5,12,14- hexaen-9-one | Suzuki reaction using 4-amino-3-bromo-2- chloropyridine (CAS: 215364-85-5) tert- butyl 2-[3-(4,4,5,5-tetramethyl-1,3,2- dioxaborolan-2-yl)-2-pyridyl]propanoate (Intermediate N31_1), K₂CO₃, bis(di-tert- butyl(4- dimethylaminophenyl)phosphine)dichloro- palladium(II) heated in dioxane/water at 100° C. followed by cyclization with LiHMDS in toluene at RT (80% yield). LC- |
| | | | MS m/z [M + H]⁺: 260.1/262.1; purity: 99%. |
| | | | ¹H NMR (400 MHz, DMSO-d₆) δ 10.77 (s, |
| | | | 1H), 8.67 (d, J = 4.6 Hz, 1H), 8.35 (d, J = |
| | | | 5.5 Hz, 1H), 8.23 (d, J = 8.0 Hz, 1H), 7.49- |
| | | | 7.42 (m, 1H), 7.23 (d, J = 5.5 Hz, 1H), |
| | | | 3.67 (q, J = 6.6 Hz, 1H), 1.48 (d, J = 6.6 |
| | | | Hz, 3H). |
| NN171 | 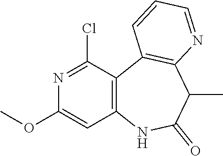 | 3-chloro-5- methoxy-10- methyl-4,8,12- triazatricyclo[9.4.0.0^{2,7}] pentadeca- 1(11),2(7),3,5,12,14- hexaen-9-one | Suzuki reaction using 3-bromo-2-chloro-6- methoxy-pyridin-4-amine (Intermediate NN170), ethyl 2-[3-(4,4,5,5-tetramethyl- 1,3,2-dioxaborolan-2-yl)-2- pyridyl]propanoate (Intermediate N31_1), K₂CO₃, bis(di-tert-butyl(4- dimethylaminophenyl)phosphine) dichloropalladium(II) heated in dioxane/ water at 100° C. followed by cyclization with LiHMDS in toluene at RT (18% yield). LC- |
| | | | MS m/z [M + H]⁺: 290.1/292.1; purity: 83%. |
| | | | ¹H NMR (400 MHz, DMSO-d₆) δ 10.60 (s, |
| | | | 1H), 8.62 (dd, J = 4.7, 1.7 Hz, 1H), 8.16 |
| | | | (dd, J = 7.9, 1.7 Hz, 1H), 7.43 (dd, J = 7.9, |
| | | | 4.7 Hz, 1H), 6.60 (s, 1H), 3.91 (s, 3H), 3.71 |
| | | | (q, J = 6.6 Hz, 1H), 1.46 (d, J = 6.6 Hz, 3H). |
| NN180 | 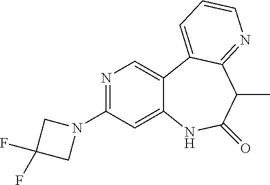 [SMILES: CC1C(=O)Nc2cc(N3CC(F)(F)C3)ncc2-c2cccnc21] | 5-(3,3- difluoroazetidin-1- yl)-10-methyl- 4,8,12- triazatricyclo[9.4.0.0^{2,7}] pentadeca- 1(11),2,4,6,12,14- hexaen-9-one | Suzuki reaction using 2-(3,3- difluoroazetidin-1-yl)-5-(4,4,5,5- tetramethyl-1,3,2-dioxaborolan-2- yl)pyridin-4-amine (Intermediate NN178), ethyl 2-(3-bromo-2-pyridyl)propanoate (Intermediate N68_2), K₃PO₄, Pd₂dba₃/ SPhos heated in dioxane/water at 90° C. followed by cyclization with LiHMDS in THF at 0° C. (33 % yield). LC-MS m/z [M + H]⁺: 317.0; purity: 99%. |
| NN196 | 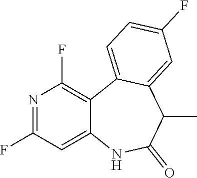 [SMILES: CC1C(=O)Nc2cc(F)nc(F)c2-c2ccc(F)cc21] | 1,3,9-trifluoro-7- methyl-5,7- dihydropyrido[4,3- d][3]benzazepin-6- one | Suzuki reaction using 2,6-difluoro-3- (4,4,5,5-tetramethyl-1,3,2-dioxaborolan-2- yl)pyridin-4-amine (Intermediate N132), methyl 2-(2-bromo-5-fluoro- phenyl)propanoate (Intermediate N70_2A) , K₃PO₄, Pd₂dba₃/SPhos heated in toluene at 100° C. followed by cyclization with LiHMDS in THE at RT (42% yield). LC-MS m/z [M − H]⁻: 277; purity: 99%. ¹H NMR (400 MHz, DMSO-d₆) δ 10.91 (s, 1H), 7.73 (dt, J = 8.4, 5.2 Hz, 1H), 7.34- |
| | | | 7.23 (m, 2H), 6.83 (s, 1H), 3.56 (q, J = 6.7 |
| | | | Hz, 1H), 1.45 (d, J = 6.7 Hz, 3H). |
| NN199 | 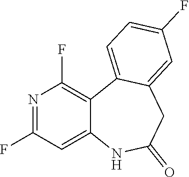 [SMILES: O=C1Cc2cc(F)ccc2-c2c(cc(F)nc2F)N1] | 1,3,9-trifluoro-5,7- dihydropyrido[4,3- d][3]benzazepin-6- one | Suzuki reaction using 2,6-difluoro-3- (4,4,5,5-tetramethyl-1,3,2-dioxaborolan-2- yl)pyridin-4-amine (Intermediate N132), 2- bromo-5-(fluorophenyl)acetic acid methyl ester, K₃PO₄, Pd₂dba₃/SPhos heated in toluene at 100° C. followed by cyclization with LiHMDS in THF at RT (73% yield). LC-MS m/z [M − H]⁻: 263; purity: 94%. ¹H NMR (400 MHz, DMSO) δ 10.88 (s, 1H), 7.73 (dt, J = 9.8, 5.3 Hz, 1H), 7.42 (dd, J = 9.4, 2.8 Hz, 1H), 7.30 (td, J = 8.7, 2.8 |
| | | | Hz, 1H), 6.85 (s, 1H), 3.67-3.53 (m, 2H). |
| NN202 | 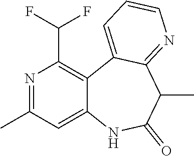 | 3-(difluoromethyl)- 5,10-dimethyl- 4,8,12- triazatricyclo[9.4.0.0^{2,7}] pentadeca- 1(11),2(7),3,5,12,14- hexaen-9-one | Suzuki reaction using 3-bromo-2- (difluoromethyl)-6-methyl-pyridin-4-amine (Intermediate NN235), tert-butyl 2-[3- (4,4,5,5-tetramethyl-1,3,2-dioxaborolan-2- yl)-2-pyridyl]propanoate (Intermediate N31_1), K₃PO₄, Pd₂dba₃/SPhos heated in toluene at 100° C. followed by cyclization with LiHMDS in THF at RT (55% yield). LC-MS m/z [M + H]⁺: 290; purity: 76%. ¹H NMR (400 MHz, CDCl₃) δ 8.89 (s, 1H), |
| | | | 8.67 (dd, J = 4.8, 1.6 Hz, 1H), 7.79-7.75 |
| | | | (m, 1H), 7.30 (dd, J = 7.9, 4.8 Hz, 1H), 7.20 |
| | | | (dd, J = 9.2, 5.9 Hz, 1H), 7.13 - 7.09 (m, |
| | | | 1H), 6.59 (t, J = 54.1 Hz, 1H), 3.55 (q, J = |
| | | | 6.7 Hz, 1H), 2.64 (s, 3H), 1.66 (d, J = 6.7 |
| | | | Hz, 3H). |
| NN204 | 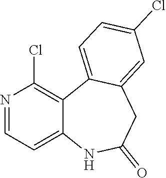 [SMILES: O=C1Cc2cc(Cl)ccc2-c2c(ccnc2Cl)N1] | 1,9-dichloro-5,7- dihydropyrido[4,3- d][3]benzazepin-6- one | Suzuki reaction using 4-amino-3-bromo-2- chloropyridine, 2-(5-chloro-2- (4,4,5,5-tetramethyl-1,3,2-dioxaborolan-2- yl)phenylacetate (CAS: 1419172-25-0), K₂CO₃, bis(di-tert-butyl(4- dimethylaminophenyl)phosphine)dichloro palladium(II) heated in dioxane/water at 100º C. followed by cyclization with LiHMDS in THF at 0° C. (40% yield). LC- MS m/z: [M + H]⁺: 279/281; purity: 93.2% |
| NN206 | 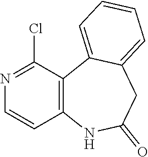 [SMILES: O=C1Cc2ccccc2-c2c(ccnc2Cl)N1] | 1-chloro-5,7- dihydropyrido[4,3- d][3]benzazepin-6- one | Suzuki reaction using 3-bromo-2-chloro- pyridin-4-amine, methyl 2-(2-(4,4,5,5- tetramethyl-1,3,2-dioxaborolan-2- yl)phenyl) acetate (CAS: 956229-86-0), K₂CO₃, bis(di-tert-butyl(4- dimethylaminophenyl)phosphine)dichloro- palladium(II) heated in dioxane/water at 100° C. followed by cyclization with LiHMDS in THF at 0° C. (42% yield). LC- MS m/z: [M + H]⁺: 243/245; purity: 98.5%. |
| NN217 | 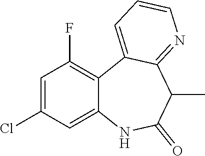 | 9-chloro-11-fluoro- 5-methyl-5,7- dihydropyrido[2,3- d][1]benzazepin-6- one | Suzuki reaction using 5-chloro-3-fluoro-2- (4,4,5,5-tetramethyl-1,3,2-dioxaborolan-2- yl)aniline (Intermediate NN215), ethyl 2- (3-bromo-2-pyridyl)propanoate (Intermediate N68_2), CsF, bis(di-tert- butyl(4- dimethylaminophenyl)phosphine)dichloro- palladium(II) heated in dioxane/water at 80° C. followed by cyclization with LiHMDS in THF at 0° C. (45% yield). LC-MS m/z: |
| | | | 277.0/279.0 [M + H]⁺; purity: 100%. ¹H |
| | | | NMR (300 MHz, DMSO-d₆) δ 10.47 (s, |
| | | | 1H), 8.66 (dd, J = 4.8, 1.7 Hz, 1H), 8.04 |
| | | | (ddd, J = 7.9, 4.6, 1.7 Hz, 1H), 7.52-7.38 |
| | | | (m, 2H), 7.16 (dd, J = 2.1, 1.5 Hz, 1H), 3.58 |
| | | | (q, J = 6.6 Hz, 1H), 1.47 (d, J = 6.7 Hz, 3H). |
| | | | 19F NMR (282 MHz, DMSO-d₆) δ −112.89 |
| | | | (ddd, J = 10.5, 4.6, 1.5 Hz |
| NN222 | 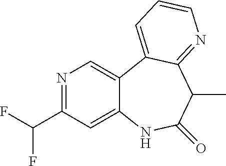 [SMILES: CC1C(=O)Nc2cc(C(F)F)ncc2-c2cccnc21] | 5-(difluoromethyl)- 10-methyl-4,8,12- triazatricyclo[9.4.0.0^{2,7}] pentadeca- 1(11),2(7),3,5,12,14- hexaen-9-one | Suzuki reaction using 2-(difluoromethyl)- 5-(4,4,5,5-tetramethyl-1,3,2-dioxaborolan- 2-yl)pyridin-4-amine (Intermediate NN231), ethyl 2-(3-bromo-2- pyridyl)propanoate (Intermediate N68_2), K₃PO₄, Pd₂dba₃/SPhos heated in dioxane/water at 80° C. followed by cyclization with LiHMDS in THF at 0° C. (77% yield). LC-MS m/z: 276.0 [M + H]⁺; purity: 100%. ¹H NMR (300 MHz, DMSO-d₆) δ 10.88 (s, 1H), 8.97 (s, 1H), 8.72 (dd, J = |
| | | | 4.8, 1.6 Hz, 1H), 8.19 (dd, J = 7.8, 1.7 Hz, |
| | | | 1H), 7.53 (dd, J = 7.8, 4.8 Hz, 1H), 7.47 (s, |
| | | | 1H), 7.28 - 6.80 (m, 1H), 3.57 (q, J = 6.5 |
| | | | Hz, 1H), 1.50 (d, J = 6.6 Hz, 3H). 19F NMR |
| | | | (282 MHz, DMSO-d₆) δ −116.03 (dd, J = |
| | | | 54.8, 21.4 Hz). |
| NN224 | 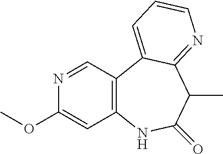 | 5-methoxy-10- methyl-4,8,12- triazatricyclo[9.4.0.0^{2,7}] pentadeca- 1(11),2(7),3,5,12,14- hexaen-9-one | Suzuki reaction using 2-methoxy-5- (4,4,5,5-tetramethyl-1,3,2-dioxaborolan-2- yl)pyridin-4-amine (Intermediate NN232), ethyl 2-(3-bromo-2-pyridyl)propanoate (Intermediate N68_2), K₃PO₄, Pd₂dba₃/ SPhos heated in dioxane/water at 80° C. followed by cyclization with LiHMDS in THF at 0° C. (41 % yield). LC-MS m/z: 256.0 [M + H]⁺; purity: 100%. ¹H NMR (300 MHz, DMSO-d₆) δ 10.56 (s, 1H), 8.62 (dd, |
| | | | J = 4.8, 1.6 Hz, 1H), 8.50 (s, 1H), 8.04 (dd, |
| | | | J = 7.8, 1.7 Hz, 1H), 7.46 (dd, J = 7.8, 4.8 |
| | | | Hz, 1H), 6.57 (s, 1H), 3.92 (s, 3H), 3.57 (q, |
| | | | J = 6.2 Hz, 1H), 1.47 (d, J = 6.7 Hz, 3H). |
| NN227 | 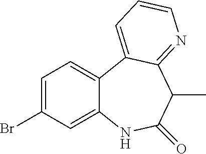 [SMILES: CC1C(=O)Nc2cc(Br)ccc2-c2cccnc21] | 9-bromo-5-methyl- 5,7- dihydropyrido[2,3- d][1]benzazepin-6- one | Suzuki reaction using 5-bromo-2-(4,4,5,5- tetramethyl-1,3,2-dioxaborolan-2- yl)aniline (Intermediate NN225) and ethyl 2-(3-bromo-2-pyridyl)propanoate (Intermediate N68_2), K₃PO₄, Pd(dppf)Cl₂ heated in toluene at 70° C. followed by cyclization with LiHMDS in THF at 0° C. (45% yield). LC-MS m/z: 303.0/305.0 [M + H]⁺; purity: 100%. ¹H NMR (300 MHz, DMSO- d₆) δ 10.35 (s, 1H), 8.65 (dd, J = 4.8, 1.7 |
| | | | Hz, 1H), 8.01 (dd, J = 7.8, 1.7 Hz, 1H), |
| | | | 7.65 (d, J = 8.4 Hz, 1H), 7.53-7.44 (m, |
| | | | 2H), 7.43 (d, J = 2.0 Hz, 1H), 3.47 (q, J = |
| | | | 6.6 Hz, 1H), 1.47 (d, J = 6.7 Hz, 3H). |
| NN229 | 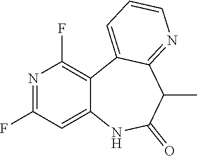 [SMILES: CC1C(=O)Nc2cc(F)nc(F)c2-c2cccnc21] | 3,5-difluoro-10- methyl-4,8,12- triazatricyclo[9.4.0.0^{2,7}] pentadeca- 1(11),2(7),3,5,12,14- hexaen-9-one | Suzuki reaction using 2,6-difluoro-3- (4,4,5,5-tetramethyl-1,3,2-dioxaborolan-2- yl)pyridin-4-amine (Intermediate N132), ethyl 2-(3-bromo-2-pyridyl)propanoate (Intermediate N68_2), K₃PO₄, Pd₂dba₃/ SPhos heated in dioxane/water at 80° C. followed by cyclization with LiHMDS in THF at RT (37 % yield, 2 steps). LC-MS m/z: 262.0 [M + H]⁺; purity: >99%. ¹H NMR (300 MHz, DMSO-d6) δ 11.04 (s, 1H), |
| | | | 8.68 (dd, J = 4.7, 1.7 Hz, 1H), 8.11 (ddd, J = |
| | | | 7.9, 4.6, 1.7 Hz, 1H), 7.47 (ddd, J = 7.9, |
| | | | 4.8, 0.5 Hz, 1H), 6.87 (d, J = 1.1 Hz, 1H), |
| | | | 3.72 (q, J = 6.6 Hz, 1H), 1.49 (d, J = 6.6 |
| | | | Hz, 3H) |
| NN317 | 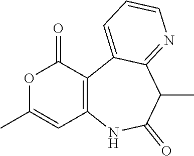 | 5,10-Dimethyl-4- oxa-8,12- diazatricyclo[9.4.0.0^{2,7}] pentadeca- 1(15),2(7),5,11,13- pentaene-3,9- dione | Suzuki reaction using 4-amino-3-bromo-6- methyl-pyran-2-one (Intermediate NN268), ethyl 2-[3-(4,4,5,5-tetramethyl- 1,3,2-dioxaborolan-2-yl)-2- pyridyl]propanoate (Intermediate N31_1), CsF, bis(di-tert-butyl(4- dimethylaminophenyl)phosphine)dichloro- palladium(II) heated in dioxane / water at 100° C. followed by cyclization with K₂CO₃ in EtOH at 80° C. (49% yield). LC-MS m/z |
| | | | [M + H]⁺: 257.2; purity: 96%. ¹H NMR (400 |
| | | | MHz, DMSO-d₆) δ 10.77 (s, 1H), 8.59 (d, J = |
| | | | 4.6 Hz, 1H), 8.14 (d, J = 8.1 Hz, 1H), |
| | | | 7.39 (t, J = 6.4 Hz, 1H), 6.20 (s, 1H), 3.54 |
| | | | (q, J = 6.6 Hz, 1H), 2.28 (s, 3H), 1.49 (d, J = |
| | | | 6.6 Hz, 3H). |
| NN318 | 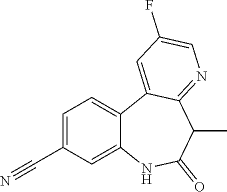 [SMILES: CC1C(=O)Nc2cc(C#N)ccc2-c2cc(F)cnc21] | 2-Fluoro-5-methyl- 6-oxo-5,7- dihydropyrido[2,3- d][1]benzazepine- 9-carbonitrile | Suzuki reaction using 3-amino-4-bromo- benzonitrile (CAS 72635-78-0), ethyl 2-[5- fluoro-3-(4,4,5,5-tetramethyl-1,3,2- dioxaborolan-2-yl)-2-pyridyl]propanoate (Intermediate NN304), CsF, SPhos Pd(crotyl)Cl heated in dioxane/water at 90° C. followed by cyclization with K₂CO₃ in EtOH at 80° C. (35% yield). LC-MS m/z [M + H]⁺: 268.0; purity: 99%. |
| NN319 | 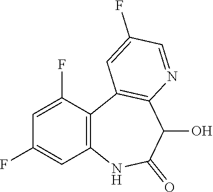 [SMILES: O=C1Nc2cc(F)cc(F)c2-c2cc(F)cnc2C1O] | 2,9,11-Trifluoro-5- hydroxy-5,7- dihydropyrido[2,3- d][1]benzazepin-6- one | Suzuki reaction using ethyl 2-acetoxy-2- (3-bromo-5-fluoro-2-pyridyl)acetate (Intermediate NN300), 3,5-difluoro-2- (4,4,5,5-tetramethyl-1,3,2-dioxaborolan-2- yl)aniline (Intermediate NN267), CsF, bis(di-tert-butyl(4- dimethylaminophenyl)phosphine)dichloro- palladium(II) heated in dioxane/water at 90° C. followed by cyclization with K₂CO₃ in EtOH at 80° C. (56% yield). LC-MS m/z [M + H]⁺: 281.1; purity: 93%. |
| NN320 | 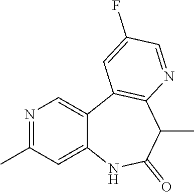 | 14-Fluoro-5,10- dimethyl-4,8,12- triazatricyclo[9.4.0.0^{2,7}] pentadeca- 1(11),2,4,6,12,14- hexaen-9-one | Suzuki reaction using 2-methyl-5-(4,4,5,5- tetramethyl-1,3,2-dioxaborolan-2- yl)pyridin-4-amine (CAS: 1668475-78-2), ethyl 2-(3-bromo-5-fluoro-2- pyridyl)propanoate (Intermediate NN213), K₂CO₃, XPHOS-Pd-G3 heated in dioxane/ water at 100° C. followed by cyclization with LiHMDS in dry THF at RT (90% yield). LC-MS m/z [M + H]⁺: 258.2; purity: 100%. ¹H NMR (400 MHz, DMSO-d₆) δ 10.66 (s, 1H), 8.76 (s, 1H), 8.67 (d, J = 2.8 Hz, 1H), 8.12 (dd, J = 9.7, 2.8 Hz, 1H), |
| | | | 7.03 (s, 1H), 3.51 (d, J = 6.6 Hz, 1H), 1.48 |
| | | | (d, J = 6.6 Hz, 3H). CH₃ protons under |
| | | | DMSO signal. |
| NN321 | 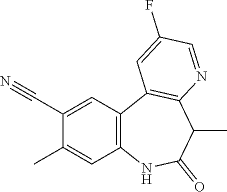 [SMILES: Cc1cc2c(cc1C#N)-c1cc(F)cnc1C(C)C(=O)N2] | 2-Fluoro-5,9- dimethyl-6-oxo- 5,7- dihydropyrido[2,3- d][1]benzazepine- 10-carbonitrile | Suzuki reaction using 4-amino-5-bromo-2- methyl-benzonitrile (Intermediate NN275), ethyl 2-[5-fluoro-3-(4,4,5,5-tetramethyl- 1,3,2-dioxaborolan-2-yl)-2- pyridyl]propanoate (Intermediate NN304), CsF, bis(di-tert-butyl(4- dimethylaminophenyl)phosphine)dichloro- palladium(II) heated in dioxane/water at 90° C. followed by cyclization with K₂CO₃ in EtOH at 80° C. (33% yield). LC-MS m/z [M + H]⁺: 282.1; purity: 98%. ¹H NMR (400 MHz, DMSO-d₆) δ 10.71 (s, 1H), 8.67 (d, J = |
| | | | 2.8 Hz, 1H), 8.22 (s, 1H), 8.12 (dd, J = |
| | | | 9.7, 2.8 Hz, 1H), 7.26 (s, 1H), 3.49 (q, J = |
| | | | 6.6 Hz, 1H), 2.53 (s, 3H), 1.47 (d, J = 6.7 |
| | | | Hz, 3H). |
| NN322 | 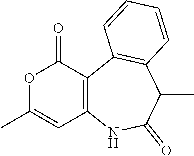 | 3,7-Dimethyl-5,7- dihydropyrano[4,3- d][3]benzazepine- 1,6-dione | Suzuki reaction using 4-amino-3-bromo-6- methyl-pyran-2-one (Intermediate NN268), ethyl 2-[2-(4,4,5,5-tetramethyl- 1,3,2-dioxaborolan-2- yl)phenyl]propanoate (Intermediate N64_2), CsF, bis(di-tert-butyl(4- dimethylaminophenyl)phosphine)dichloro- palladium(II) heated in dioxane/water at 90° C. followed by cyclization with K₂CO₃ in EtOH at 80° C. (48 % yield). LC-MS m/z |
| | | | [M + H]⁺: 256.2; purity: 99%. |
| NN323 | 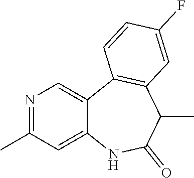 | 9-Fluoro-3,7- dimethyl-5,7- dihydropyrido[4,3- d][3]benzazepin-6- one | Suzuki reaction using ethyl 2-[5-fluoro-2- (4,4,5,5-tetramethyl-1,3,2-dioxaborolan-2- yl)phenyl]propanoate (Intermediate N70_3), 5-bromo-2-methyl-pyridin-4- amine (CAS 10460-50-1), CsF, bis(di-tert- butyl(4- dimethylaminophenyl)phosphine)dichloro- palladium(II) heated in dioxane/water at 90° C. followed by cyclization with LiHMDS in THF at 0° C. (55% yield). LC-MS m/z [M + H]⁺: 257.2; purity: 100%. |
| NN324 | 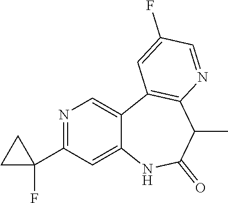 [SMILES: CC1C(=O)Nc2cc(C3(F)CC3)ncc2-c2cc(F)cnc21] | 14-Fluoro-5-(1- fluorocyclopropyl)- 10-methyl-4,8,12- triazatricyclo[9.4.0.0^{2,7}] pentadeca- 1(11),2,4,6,12,14- hexaen-9-one | Suzuki reaction using 5-bromo-2-(1- fluorocyclopropyl)pyridin-4-amine (Intermediate NN277), ethyl 2-[5-fluoro-3- (4,4,5,5-tetramethyl-1,3,2-dioxaborolan-2- yl)-2-pyridyl]propanoate (Intermediate NN304), CsF, bis(di-tert-butyl(4- dimethylaminophenyl)phosphine)dichloro- palladium(II) heated in dioxane/water at 95° C. followed by cyclization with K₂CO₃ in EtOH at 80 ° C. (60% yield). LC-MS (Method 1) m/z [M + H]⁺: 302.2; rt: 1.00 min; purity: 100%. ¹H NMR (400 MHz, DMSO- d₆) δ 11.95 (s, 1H), 8.81 (s, 1H), 8.69 (d, J = |
| | | | 2.8 Hz, 1H), 8.14 (dd, J = 9.7, 2.8 Hz, |
| | | | 1H), 7.42 (d, J = 1.7 Hz, 1H), 3.56 (d, J = |
| | | | 7.1 Hz, 1H), 1.58 (dd, J = 19.1, 3.0 Hz, |
| | | | 2H), 1.49 (d, J = 6.6 Hz, 3H), 1.41 (dd, J = |
| | | | 8.8, 4.1 Hz, 2H); purity: 80%. |
| NN325 | 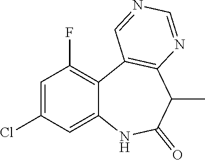 | 9-Chloro-11- fluoro-5-methyl- 5,7- dihydropyrimido[4,5- d][1]benzazepin- 6-one | Suzuki reaction using ethyl 2-(5- bromopyrimidin-4-yl)propanoate (Intermediate N36_3), 5-chloro-3-fluoro-2- (4,4,5,5-tetramethyl-1,3,2-dioxaborolan-2- yl)aniline (Intermediate NN286), K₂CO₃, bis(di-tert-butyl(4- dimethylaminophenyl)phosphine)dichloro- palladium(II) heated in dioxane/water at 100° C. followed by cyclization with K₂CO₃ in EtOH at 80 ° C. (57% yield). LC-MS m/z |
| | | | [M + H]⁺: 278.2, 280.0; purity: 96%. |
| NN326 | 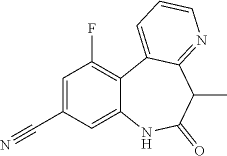 [SMILES: CC1C(=O)Nc2cc(C#N)cc(F)c2-c2cccnc21] | 11-Fluoro-5- methyl-6-oxo-5,7- dihydropyrido[2,3- d][1]benzazepine- 9-carbonitrile | Suzuki reaction using ethyl 2-(3-bromo-2- pyridyl)propanoate, 3-amino-5-fluoro-4- (4,4,5,5-tetramethyl-1,3,2-dioxaborolan-2- yl)benzonitrile (Intermediate NN233), CsF, bis(di-tert-butyl(4- dimethylaminophenyl)phosphine)dichloro- palladium(II) heated in dioxane/water at reflux followed by cyclization with K₂CO₃ in EtOH at 80 ° C. (4% yield). LC-MS m/z [M + H]⁺: 268.2; purity: 61%. |
| NN327 | 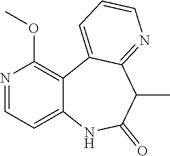 [SMILES: COc1nccc2c1-c1cccnc1C(C)C(=O)N2] | 3-Methoxy-10- methyl-4,8,12- triazatricyclo[9.4.0.0^{2,7}] pentadeca- 1(11),2(7),3,5,12,14- hexaen-9-one | Suzuki reaction using 3-bromo-2- methoxy-pyridin-4-amine (CAS 215364- 86-6), ethyl 2-[3-(4,4,5,5-tetramethyl- 1,3,2-dioxaborolan-2-yl)-2- pyridyl]propanoate (Intermediate N31_1), CsF, SPhos Pd(crotyl)Cl heated in dioxane/water at 90° C. followed by cyclization with K₂CO₃ in EtOH at 80° C. (38% yield). LC-MS m/z [M + H]⁺: 256.2; purity: 96%. ¹H NMR (400 MHz, DMSO-d₆) |
| | | | δ 10.53 (s, 1H), 8.59 (dd, J = 4.7, 1.7 Hz, |
| | | | 1H), 8.14 (dd, J = 8.0, 1.7 Hz, 1H), 8.11 (d, |
| | | | J = 5.6 Hz, 1H), 7.39 (dd, J = 7.9, 4.7 Hz, |
| | | | 1H), 6.86 (d, J = 5.6 Hz, 1H), 3.91 (s, 3H), |
| | | | 3.48 (q, J = 6.6 Hz, 1H), 1.47 (d, J = 6.7 |
| | | | Hz, 3H). |
| NN328 | 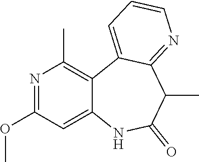 | 5-Methoxy-3,10- dimethyl-4,8,12- triazatricyclo[9.4.0.0^{2,7}] pentadeca- 1(15),2,4,6,11,13- hexaen-9-one | Suzuki reaction using 3-bromo-6- methoxy-2-methyl-pyridin-4-amine (Intermediate #355_2) ethyl 2-[3-(4,4,5,5- tetramethyl-1,3,2-dioxaborolan-2-yl)-2- pyridyl]propanoate (Intermediate N31_1), CsF, bis(di-tert-butyl(4- dimethylaminophenyl)phosphine)dichloro- palladium(II) heated in dioxane/water at reflux followed by cyclization with K₂CO₃ in EtOH at 80° C. (4% yield). LC-MS m/z |
| | | | [M + H]⁺: 270.2; purity: 49%. ¹H NMR (400 |
| | | | MHz, DMSO-d₆) δ 10.32 (s, 1H), 8.58 (dd, |
| | | | J = 4.7, 1.7 Hz, 1H), 7.98 (dd, J = 7.8, 1.7 |
| | | | Hz, 1H), 7.40 (dd, J = 7.8, 4.7 Hz, 1H), |
| | | | 6.44 (s, 1H), 3.89 (s, 3H), 3.62 (q, J = 6.6 |
| | | | Hz, 1H), 2.51 (s, 3H), 1.46 (d, J = 6.7 Hz, |
| | | | 3H). |
| NN329 | 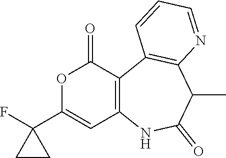 | 5-(1- fluorocyclopropyl)- 10-methyl-4-oxa- 8,12- diazatricyclo[9.4.0.0^{2,7}] pentadeca- 1(15),2(7),5,11,13- pentaene-3,9- dione | Suzuki reaction using 4-amino-3-bromo-6- (1-fluorocyclopropyl)pyran-2-one (Intermediate NN254), ethyl 2-[3-(4,4,5,5- tetramethyl-1,3,2-dioxaborolan-2-yl)-2- pyridyl]propanoate (Intermediate N31_1), CsF, bis(di-tert-butyl(4- dimethylaminophenyl)phosphine)dichloro- palladium(II) heated in dioxane/water at 90° C. followed by cyclization with K₂CO₃ in EtOH at 80° C. (18% yield). LC-MS m/z |
| | | | [M + H]⁺: 301.1; purity: 49%. |
| NN330 | 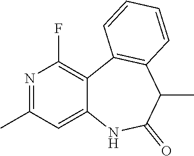 | 1-Fluoro-3,7- dimethyl-5,7- dihydropyrido[4,3- d][3]benzazepin-6- one | Suzuki reaction using ethyl 2-(2- bromophenyl)propanoate (Intermediate N64_1), 2-fluoro-6-methyl-3-(4,4,5,5- tetramethyl-1,3,2-dioxaborolan-2- yl)pyridin-4-amine (Intermediate N16_1), K₂CO₃, bis(di-tert-butyl(4- dimethylaminophenyl)phosphine)dichloro- palladium(II) heated in dioxane/water at 100° C. followed by cyclization with K2CO3 in EtOH at 80° C. (19% yield). LC-MS m/z |
| | | | [M + H]⁺: 257.2; purity: 99%. |
| NN331 | 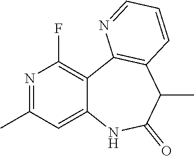 | 15-Fluoro-8,13- dimethyl-3,10,14- triazatricyclo[9.4.0.0^{2,7}] pentadeca- 1(11),2(7),3,5,12,14- hexaen-9-one | Suzuki reaction using 2-fluoro-6-methyl-3- (4,4,5,5-tetramethyl-1,3,2-dioxaborolan-2- yl)pyridin-4-amine (Intermediate N16_1), ethyl 2-(2-chloro-3-pyridyl)propanoate (Intermediate NN296), CsF, bis(di-tert- butyl(4- dimethylaminophenyl)phosphine)dichloro- palladium(II) heated in dioxane/water at 90° C. followed by cyclization with K₂CO₃ in EtOH at 80° C. (25% yield). LC-MS m/z |
| | | | [M + H]⁺: 258.2; purity: 97%. |
| NN332 | 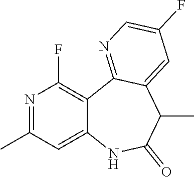 | 5,15-Difluoro-8,13- dimethyl-3,10,14- triazatricyclo[9.4.0.0^{2,7}] pentadeca- 1(11),2(7),3,5,12,14- hexaen-9-one | Suzuki reaction using methyl 2-(2-chloro- 5-fluoro-3-pyridyl)propanoate (Intermediate NN293), 2-fluoro-6-methyl- 3-(4,4,5,5-tetramethyl-1,3,2-dioxaborolan- 2-yl)pyridin-4-amine (Intermediate N16_1), CsF, bis(di-tert-butyl(4- dimethylaminophenyl)phosphine)dichloro- palladium(II) heated in dioxane/water at 90° C. followed by cyclization with K₂CO₃ in EtOH at 80° C. (63% yield). LC-MS m/z [M + H]⁺: 276.1; purity: 86%. ¹H NMR (400 |
| | | | MHz, DMSO-d₆) δ 10.77 (s, 1H), 8.65 (d, J = |
| | | | 2.7 Hz, 1H), 7.78 (dd, J = 9.7, 2.8 Hz, |
| | | | 1H), 6.95 (s, 1H), 3.58 (q, J = 6.7 Hz, 1H), |
| | | | 2.44 (s, 3H), 1.46 (d, J = 6.8 Hz, 3H). |
| NN333 | 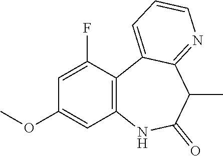 | 11-Fluoro-9- methoxy-5-methyl- 5,7- dihydropyrido[2,3- d][1]benzazepin-6- one | Suzuki reaction using ethyl 2-(3-bromo-2- pyridyl)propanoate (Intermediate N68_2), 3-fluoro-5-methoxy-2-(4,4,5,5- tetramethyl-1,3,2-dioxaborolan-2- yl)aniline (Intermediate NN272), CsF, bis(di-tert-butyl(4- dimethylaminophenyl)phosphine)dichloro- palladium(II) heated in dioxane/water at 90° C. followed by cyclization with K₂CO₃ in EtOH at 80° C. (43% yield). LC-MS m/z |
| | | | [M + H]⁺: 273.1; purity: 98%. |
| NN334 | 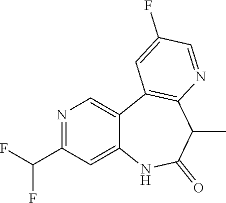 [SMILES: CC1C(=O)Nc2cc(C(F)F)ncc2-c2cc(F)cnc21] | 5-(Difluoromethyl)- 14-fluoro-10- methyl-4,8,12- triazatricyclo[9.4.0.0^{2,7}] pentadeca- 1(11),2,4,6,12,14- hexaen-9-one | Suzuki reaction using 5-bromo-2- (difluoromethyl)pyridin-4-amine (Intermediate NN246), ethyl 2-[5-fluoro-3- (4,4,5,5-tetramethyl-1,3,2-dioxaborolan-2- yl)-2-pyridyl]propanoate (Intermediate NN304), CsF, bis(di-tert-butyl(4- dimethylaminophenyl)phosphine)dichloro- palladium(II) heated in dioxane/water at 90° C. followed by cyclization with K₂CO₃ in EtOH at 80° C. (81% yield). LC-MS m/z [M + H]⁺: 294.1; purity: 98%. |
| NN335 | 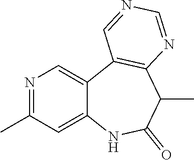 | 8,13-Dimethyl- 4,6,10,14- tetrazatricyclo[9.4.0.0^{2,7}] pentadeca- 1(15),2(7),3,5,11,13- hexaen-9-one | Suzuki reaction using ethyl 2-(5- bromopyrimidin-4-yl)propanoate (Intermediate N36_3), 2-methyl-5- (4,4,5,5-tetramethyl-1,3,2-dioxaborolan-2- yl)pyridin-4-amine (CAS 1668475-78-2), CsF, bis(di-tert-butyl(4- dimethylaminophenyl)phosphine)dichloro- palladium(II) heated in dioxane/water at 100° C. followed by cyclization with K₂CO₃ (13% yield). LC-MS m/z [M + H]⁺: 241.1; |
| | | | purity: 98%. ¹H NMR (400 MHz, DMSO-d₆) |
| | | | δ 10.79 (s, 1H), 9.25 (s, 1H), 9.10 (s, 1H), |
| | | | 8.84 (s, 1H), 7.06 (s, 1H), 3.64 (q, J = 6.6 |
| | | | Hz, 1H), 2.53 (s, 3H), 1.47 (d, J = 6.7 Hz, |
| | | | 3H). |
| NN152 | 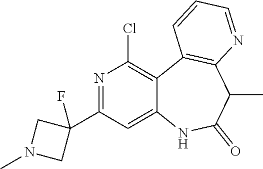 | 3-chloro-5-(3- fluoro-1-methyl- azetidin-3-yl)-10- methyl-4,8,12- triazatricyclo[9.4.0.0^{2,7}] pentadeca- 1(11),2,4,6,12,14- hexaen-9-one | Suzuki reaction using 3-bromo-2-chloro-6- (3-fluoro-1-methyl-azetidin-3-yl)pyridin-4- amine (Intermediate NN151), ethyl 2-[3- (4,4,5,5-tetramethyl-1,3,2-dioxaborolan-2- yl)-2-pyridyl]propanoate (Intermediate N31_1), CsF, bis(di-tert-butyl(4- dimethylaminophenyl)phosphine)dichloro- palladium(II) heated in dioxane/water at 100° C. followed by cyclization with LiHMDS (51% yield). LC-MS m/z [M + H]⁺: 347.0/348.7; purity: 90%. LC-MS m/z |
| | | | [M + H]⁺: 347.1/349.1; purity: 87%. ¹H NMR |
| | | | (400 MHz, DMSO-d₆) δ 10.82 (s, 1H), 8.68 |
| | | | (dd, J = 4.7, 1.7 Hz, 1H), 8.25 (dd, J = 8.0, |
| | | | 1.7 Hz, 1H), 7.47 (dd, J = 8.0, 4.7 Hz, 1H), |
| | | | 7.37 (s, 1H), 3.79-3.56 (m, 5H), 2.40 (s, |
| | | | 3H), 1.48 (d, J = 6.6 Hz, 3H). 19F NMR |
| | | | (376 MHz, DMSO-d₆) δ −152.98 (p, J = |
| | | | 18.8 Hz). Selective HOESY NMR (400 |
| | | | MHz, DMSO-d₆) δ 7.38 (s), 3.65 (d, J = 7.8 |
| | | | Hz), 3.60 (d, J = 10.0 Hz). |
| NN336 | 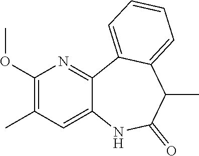 | 2-methoxy-3,7- dimethyl-5,7- dihydropyrido[2,3- a][3]benzazepin-6- one | Suzuki reaction using 2-bromo-6- methoxy-5-methyl-pyridin-3-amine, ethyl 2-[2-(4,4,5,5-tetramethyl-1,3,2- dioxaborolan-2-yl)phenyl]propanoate (Intermediate N64_2), CsF, bis(di-tert- butyl(4- dimethylaminophenyl)phosphine)dichloro- palladium(II) heated in dioxane/water at 90° C. followed by cyclization with K₂CO₃ in EtOH at 40° C. (43% yield). LC-MS m/z |
| | | | [M + H]⁺: 269.2; purity: 98%. ¹H NMR (400 |
| | | | MHz, DMSO-d₆) δ 9.96 (s, 1H), 7.97-7.90 |
| | | | (m, 1H), 7.52 (td, J = 7.8, 1.5 Hz, 1H), 7.47- |
| | | | 7.41 (m, 1H), 7.37-7.31 (m, 2H), 3.97 |
| | | | (s, 3H), 3.25 (t, J = 6.8 Hz, 1H), 2.24-2.18 |
| | | | (m, 3H), 1.46 (d, J = 6.9 Hz, 3H). |
| NN337 | 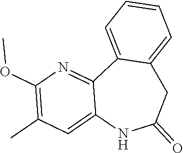 [SMILES: COc1nc2c(cc1C)NC(=O)Cc1ccccc1-2] | 2-methoxy-3- methyl-5,7- dihydropyrido[2,3- a][3]benzazepin-6- one | Suzuki reaction using 2-bromo-6- methoxy-5-methyl-pyridin-3-amine (Intermediate NN285), ethyl 2-[2-(4,4,5,5- tetramethyl-1,3,2-dioxaborolan-2- yl)phenyl]acetate (CAS 1228690-72-9), CsF, bis(di-tert-butyl(4- dimethylaminophenyl)phosphine)dichloro- palladium(II) heated in dioxane/water at reflux (17% yield). LC-MS m/z [M + H]⁺: 255.1; purity: 80%. ¹H NMR (400 MHz, |
| | | | DMSO-d₆) δ 9.92 (s, 1H), 8.01-7.91 (m, |
| | | | 1H), 7.51-7.42 (m, 2H), 7.42-7.31 (m, |
| | | | 2H), 3.97 (s, 3H), 3.78 (s, 2H), 2.20 (d, J = |
| | | | 0.9 Hz, 3H). |
| NN338 | 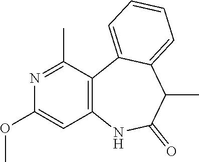 | 3-methoxy-1,7- dimethyl-5,7- dihydropyrido[3,4- a][3]benzazepin-6- one | Suzuki reaction using ethyl 2-[2-(4,4,5,5- tetramethyl-1,3,2-dioxaborolan-2- yl)phenyl]propanoate (Intermediate N64_2), 4-bromo-6-methoxy-5-methyl- pyridin-3-amine #355_2 , K₂CO₃, bis(di- tert-butyl(4- dimethylaminophenyl)phosphine)dichloro- palladium(II) heated in dioxane/water at 80° C. followed by cyclization with K₂CO₃ in EtOH at RT (24% yield). LC-MS m/z |
| | | | [M + H]⁺: 269.2; purity: 83%. |
| NN339 | 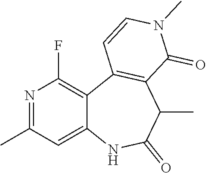 [SMILES: Cc1cc2c(c(F)n1)-c1ccn(C)c(=O)c1C(C)C(=O)N2] | 3-fluoro-5,10,13- trimethyl-4,8,13- triazatricyclo[9.4.0.0^{2,7}] pentadeca- 1(11),2,4,6,14- pentaene-9,12- dione | Suzuki reaction using ethyl 2-(4-chloro-1- methyl-2-oxo-3-pyridyl)propanoate (Intermediate #356_6), 2-fluoro-6-methyl- 3-(4,4,5,5-tetramethyl-1,3,2-dioxaborolan- 2-yl)pyridin-4-amine Intermediate N16_1, CsF, XPhos Pd(crotyl)Cl heated in dioxane/water at reflux followed by cyclization with K₂CO₃ in EtOH at RT (42% yield). LC-MS m/z [M + H]⁺: 288.2; purity: 95%. ¹H NMR (400 MHz, DMSO-d₆) δ 10.86 (bs, 1H), 7.64 (d, J = 7.3 Hz, 1H), |
| | | | 6.95 (s, 1H), 6.44-6.37 (m, 1H), 4.67 (q, |
| | | | J = 7.7 Hz, 1H), 3.48 (s, 3H), 2.42 (s, 3H), |
| | | | 0.89 (d, J = 7.7 Hz, 3H). |
| NN340 | 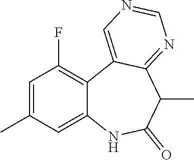 | 11-fluoro-5,9- dimethyl-5,7- dihydropyrimido[4,5- d][1]benzazepin- 6-one | Suzuki reaction using 3-fluoro-5-methyl-2- (4,4,5,5-tetramethyl-1,3,2-dioxaborolan-2- yl)aniline (Intermediate #370_1), ethyl 2- (5-bromopyrimidin-4-yl)propanoate (Intermediate N36_3), CsF, bis(di-tert- butyl(4- dimethylaminophenyl)phosphine)dichloro- palladium(II) heated in dioxane / water at 100° C. followed by cyclization with K₂CO₃ in EtOH at RT. LC-MS m/z [M + H]⁺: 258.2; |
| | | | purity: 98%. |
| NN341 | 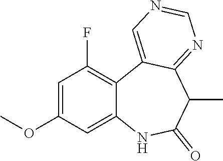 | 11-fluoro-9- methoxy-5-methyl- 5,7- dihydropyrimido[4,5- d][1]benzazepin- 6-one | Suzuki reaction using ethyl 2-(5- bromopyrimidin-4-yl)propanoate Intermediate N36_3, 3-fluoro-5-methoxy- 2-(4,4,5,5-tetramethyl-1,3,2-dioxaborolan- 2-yl)aniline Intermediate NN272, CsF, bis(di-tert-butyl(4- dimethylaminophenyl)phosphine)dichloro- palladium(II) heated in dioxane/water at 100° C. followed by cyclization with K₂CO₃ in EtOH at 80° C. (49% yield). LC-MS m/z |
| | | | [M + H]⁺: 274.2; purity: 100%. ¹H NMR (400 |
| | | | MHz, DMSO-d₆) δ 10.48 (s, 1H), 9.18 (s, |
| | | | 1H), 8.98 (d, J = 4.6 Hz, 1H), 6.96 (dd, J = |
| | | | 12.7, 2.5 Hz, 1H), 6.85 - 6.42 (m, 1H), |
| | | | 3.84 (s, 3H), 3.64 (q, J = 6.6 Hz, 1H), 1.46 |
| | | | (d, J = 6.6 Hz, 3H). |
| NN342 | 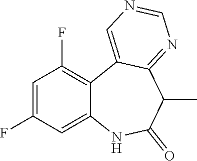 | 9,11-difluoro-5- methyl-5,7- dihydropyrimido[4,5- d][1]benzazepin- 6-one | Suzuki reaction using ethyl 2-(5- bromopyrimidin-4-yl)propanoate Intermediate N36_3, 3,5-difluoro-2- (4,4,5,5-tetramethyl-1,3,2-dioxaborolan-2- yl)aniline Intermediate NN267, CsF, bis(di-tert-butyl(4- dimethylaminophenyl)phosphine)dichloro- palladium(II) heated in dioxane/water at 100° C. followed by cyclization with K₂CO₃ in EtOH at 80° C. (85% yield). LC-MS m/z |
| | | | [M + H]⁺: 262.0; purity: 94%. |
| NN343 | 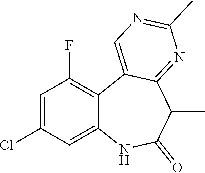 [SMILES: Cc1ncc2c(n1)C(C)C(=O)Nc1cc(Cl)cc(F)c1-2] | 9-chloro-11-fluoro- 3,5-dimethyl-5,7- dihydropyrimido[4,5- d][1]benzazepin- 6-one | Suzuki reaction using ethyl 2-(5-bromo-2- methyl-pyrimidin-4-yl)propanoate Intermediate NN305, 5-chloro-3-fluoro-2- (4,4,5,5-tetramethyl-1,3,2-dioxaborolan-2- yl)aniline Intermediate NN286, CsF, bis(di- tert-butyl(4- dimethylaminophenyl)phosphine)dichloro- palladium(II) heated in dioxane/water at 90° C. followed by cyclization with LiHMDS in THF at 0° C. (24 % yield). LC-MS m/z [M + H]⁺: 292.0, 294.2; purity: 95%. ¹H NMR |
| | | | (400 MHz, DMSO-d₆) δ 10.62 (s, 1H), 8.91 |
| | | | (d, J = 4.6 Hz, 1H), 7.49 (dd, J = 10.3, 1.9 |
| | | | Hz, 1H), 7.18 (t, J = 1.9 Hz, 1H), 3.64 (q, J = |
| | | | 6.6 Hz, 1H), 2.69 (s, 3H), 1.45 (d, J = 6.6 |
| | | | Hz, 3H). |
| NN345 | 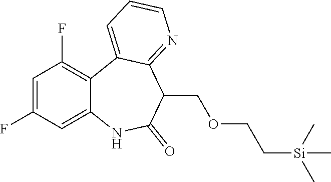 [SMILES: C[Si](C)(C)CCOCC1C(=O)Nc2cc(F)cc(F)c2-c2cccnc21] | 9,11-difluoro-5-(2- trimethylsilylethoxy- methyl)-5,7- dihydropyrido[2,3- d][1]benzazepin-6- one | Suzuki reaction using ethyl 2-(3-bromo-2- pyridyl)-3-(2- trimethylsilylethoxy)propanoate Intermediate NN299 and 3,5-difluoro-2- (4,4,5,5-tetramethyl-1,3,2-dioxaborolan-2- yl)aniline (Intermediate NN267), CsF, SPhos Pd(crotyl)Cl heated in dioxane/ water at 90° C. followed by cyclization with K₂CO₃ in EtOH at 80° C. LC-MS m/z [M + H]⁺: 377.1,; purity: 96%. ¹H NMR (400 MHz, DMSO-d₆) δ 10.56 (s, 1H), 8.62 (dd, J = 4.8, 1.7 Hz, 1H), 8.05 (ddd, J = 8.1, 4.6, |
| | | | 1.7 Hz, 1H), 7.45 (dd, J = 7.9, 4.7 Hz, 1H), |
| | | | 7.37-7.26 (m, 1H), 6.95 (d, J = 9.8 Hz, |
| | | | 1H), 4.27 - 4.14 (m, 2H), 3.57 - 3.49 (m, |
| | | | 3H), 0.81 (dd, J = 8.7, 7.0 Hz, 2H), −0.05 |
| | | | (s, 9H). |
| NN346 | 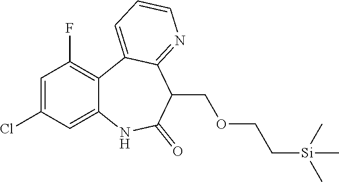 [SMILES: C[Si](C)(C)CCOCC1C(=O)Nc2cc(Cl)cc(F)c2-c2cccnc21] | 9-chloro-11-fluoro- 5-(2- trimethylsilylethoxy methyl)-5,7- dihydropyrido[2,3- d][1]benzazepin-6- one | Suzuki reaction using ethyl 2-(3-bromo-2- pyridyl)-3-(2- trimethylsilylethoxy)propanoate (Intermediate NN299), 5-chloro-3-fluoro- 2-(4,4,5,5-tetramethyl-1,3,2-dioxaborolan- 2-yl)aniline (Intermediate NN286), CsF, SPhos Pd(crotyl)Cl heated in dioxane/ water at 90° C. followed by cyclization with K₂CO₃ in EtOH at 80° C. (25% yield). LC- MS m/z [M + H]⁺: 393.3, 395.3; purity: 98%. ¹H NMR (400 MHz, DMSO- d6) δ 10.56 (s, 1H), 8.63 (dd, J = 4.8, 1.7 Hz, 1H), 8.07 |
| | | | (ddd, J = 8.0, 4.5, 1.7 Hz, 1H), 7.51-7.40 |
| | | | (m, 2H), 7.22-7.11 (m, 1H), 4.31-4.15 |
| | | | (m, 2H), 3.53 (ddd, J = 7.3, 6.3, 4.5 Hz, |
| | | | 3H), 0.84-0.78 (m, 2H), −0.05 (s, 9H). |
| NN347 | 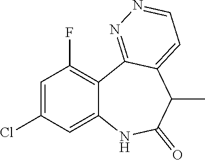 | 9-chloro-11-fluoro- 5-methyl-5,7- dihydropyridazino[4,3- d][1]benzazepin-6- one | Suzuki reaction using ethyl 2-(3- chloropyridazin-4-yl)propanoate (Intermediate NN308), 5-chloro-3-fluoro- 2-(4,4,5,5-tetramethyl-1,3,2-dioxaborolan- 2-yl)aniline (Intermediate NN286), CsF, bis(di-tert-butyl(4- dimethylaminophenyl)phosphine)dichloro- palladium(II) heated in dioxane/water at 90° C. followed by cyclization with K₂CO₃ in EtOH at 80° C. (48% yield). LC-MS m/z |
| | | | [M + H]⁺: 278.1, 280.0; purity: 93%. ¹H NMR |
| | | | (400 MHz, DMSO-d₆) δ 10.56 (s, 1H), 9.30 |
| | | | (d, J = 5.4 Hz, 1H), 7.74 (dd, J = 5.5, 1.0 |
| | | | Hz, 1H), 7.49 (dd, J = 10.2, 2.1 Hz, 1H), |
| | | | 7.18 (t, J = 1.7 Hz, 1H), 3.69-3.59 (m, |
| | | | 1H), 1.45 (d, J = 6.7 Hz, 3H). 19F NMR |
| | | | (376 MHz, DMSO-d₆) δ −111.00. |
| NN348 | 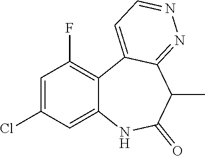 | 9-chloro-11-fluoro- 5-methyl-5,7- dihydropyridazino[3,4- d][1]benzazepin-6- one | Suzuki reaction using ethyl 2-(4- chloropyridazin-3-yl)propanoate (Intermediate NN311), 5-chloro-3-fluoro- 2-(4,4,5,5-tetramethyl-1,3,2-dioxaborolan- 2-yl)aniline (Intermediate NN286), CsF, bis(di-tert-butyl(4- dimethylaminophenyl)phosphine)dichloro- palladium(II) heated in dioxane/water at 90° C. followed by cyclization with K₂CO₃ in EtOH at 80° C. (20% yield). LC-MS m/z |
| | | | [M + H]⁺: 278.1, 280.0; purity: 92%. ¹H NMR |
| | | | (400 MHz, DMSO-d₆) δ 10.64 (s, 1H), 9.31 |
| | | | (d, J = 5.3 Hz, 1H), 8.02-7.91 (m, 1H), |
| | | | 7.52 (dd, J = 10.5, 2.1 Hz, 1H), 7.19 (t, J = |
| | | | 1.7 Hz, 1H), 3.87 (q, J = 6.6 Hz, 1H), 1.62 |
| | | | (d, J = 6.7 Hz, 3H). |
| NN349 | 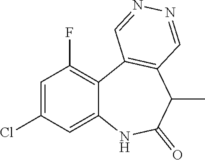 | 9-chloro-11-fluoro- 5-methyl-5,7- dihydropyridazino[4,5- d][1]benzazepin-6- one | Suzuki reaction using 5-chloro-3-fluoro-2- (4,4,5,5-tetramethyl-1,3,2-dioxaborolan-2- yl)aniline (Intermediate NN286), ethyl 2- (5-chloropyridazin-4-yl)propanoate (Intermediate NN314), CsF, bis(di-tert- butyl(4- dimethylaminophenyl)phosphine)dichloro- palladium(II) heated in dioxane/water at 90° C. followed by cyclization with K₂CO₃ in EtOH at 80° C. (34% yield). LC-MS m/z |
| | | | [M + H]⁺: 278.0, 280.2; purity: 96%. |
| NN350 | 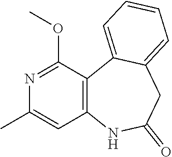 [SMILES: COc1nc(C)cc2c1-c1ccccc1CC(=O)N2] | 1-methoxy-3- methyl-5,7- dihydropyrido[4,3- d][3]benzazepin-6- one | Suzuki reaction using 3-bromo-2- methoxy-6-methyl-pyridin-4-amine (Intermediate NN274), methyl 2-[2- (4,4,5,5-tetramethyl-1,3,2-dioxaborolan-2- yl)phenyl]acetate (CAS: 956229-86-0), CsF, Palladium(II) acetate/SPhos heated in dioxane/water at reflux followed by cyclization with K₂CO₃ in EtOH at 80° C. (26% yield). LC-MS m/z [M + H]⁺: 255.1; purity: 90%. ¹H NMR (400 MHz, DMSO-d₆) δ |
| | | | 10.23 (s, 1H), 7.70 (dd, J = 6.8, 1.7 Hz, |
| | | | 1H), 7.44-7.27 (m, 3H), 6.66 (s, 1H), 3.87 |
| | | | (s, 3H), 3.44 - 3.36 (m, 2H), 2.40 (s, 3H). |
| NN351 | 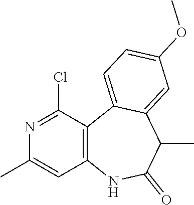 | 1-chloro-9- methoxy-3,7- dimethyl-5,7- dihydropyrido[3,4- a][3]benzazepin-6- one | Suzuki reaction using ethyl 2-[5-methoxy- 2-(4,4,5,5-tetramethyl-1,3,2-dioxaborolan- 2-yl)phenyl]propanoate (Intermediate #393_2), and 3-bromo-2-chloro-6-methyl- pyridin-4-amine (Intermediate N30_1), K₂CO₃, Pd(dppf)Cl2 heated in dioxane/ water at 90° C. followed by cyclization with LiHMDS in THF at 0° C. (39% yield). LC- MS m/z [M + H]⁺: 303.1, 305.1; purity: 85%. |
| NN352 | 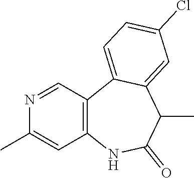 | 9-chloro-3,7- dimethyl-5,7- dihydropyrido[4,3- d][3]benzazepin-6- one | Suzuki reaction using methyl 2-[5-chloro- 2-(4,4,5,5-tetramethyl-1,3,2-dioxaborolan- 2-yl)phenyl] propanoate (Intermediate N73_2), 5-bromo-2-methyl-pyridin-4- amine (CAS 10460-50-1), CsF, bis(di-tert- butyl(4- dimethylaminophenyl)phosphine)dichloro palladium(II) heated in dioxane/water at 90° C. followed by cyclization with K₂CO₃ in EtOH at 80 (51% yield). LC-MS m/z [M + H]⁺: 273.2; purity: 77%. ¹H NMR (400 |
| | | | MHz, DMSO-d₆) δ 10.51 (s, 1H), 8.68 (s, |
| | | | 1H), 7.66 (d, J = 8.3 Hz, 1H), 7.51 (dd, J = |
| | | | 8.4, 2.1 Hz, 1H), 7.38 (d, J = 2.2 Hz, 1H), |
| | | | 6.97 (s, 1H), 3.34 (q, J = 6.8 Hz, 1H), 1.45 |
| | | | (d, J = 6.8 Hz, 3H). CH₃ obscured by |
| | | | solvent peak. |
| NN353 | 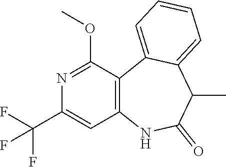 [SMILES: COc1nc(C(F)(F)F)cc2c1-c1ccccc1C(C)C(=O)N2] | 1-methoxy-7- methyl-3- (trifluoromethyl)- 5,7- dihydropyrido[4,3- d][3]benzazepin-6- one | Suzuki reaction using 3-bromo-2- methoxy-6-(trifluoromethyl)pyridin-4- amine (Intermediate #397_2), ethyl 2-[2- (4,4,5,5-tetramethyl-1,3,2-dioxaborolan-2- yl)phenyl]propanoate (Intermediate N64_2), CsF, bis(di-tert-butyl(4- dimethylaminophenyl)phosphine)dichloro- palladium(II) heated in dioxane/water at 90° C. followed by cyclization with K₂CO₃ in EtOH at 80° C. (56% yield). LC-MS m/z [M + H]⁺: 323.1; purity: 73%. |
| NN354 | 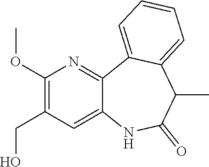 [SMILES: COc1nc2c(cc1CO)NC(=O)C(C)c1ccccc1-2] | 3-(hydroxymethyl)- 2-methoxy-7- methyl-5,7- dihydropyrido[3,2- d][3]benzazepin-6- one | Suzuki reaction using (5-amino-6-bromo- 2-methoxy-3-pyridyl)methanol (Intermediate NN247), ethyl 2-[2-(4,4,5,5- tetramethyl-1,3,2-dioxaborolan-2- yl)phenyl]propanoate (Intermediate N64_2), CsF, bis(di-tert-butyl(4- dimethylaminophenyl)phosphine)dichloro palladium(II) heated in dioxane/water at 100° C. (25% yield). LC-MS m/z [M + H]⁺: 285.2; purity: 97%. |
| NN355 | 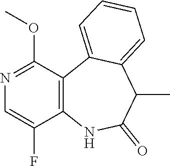 [SMILES: COc1ncc(F)c2c1-c1ccccc1C(C)C(=O)N2] | 4-fluoro-1- methoxy-7-methyl- 5,7- dihydropyrido[4,3- d][3]benzazepin-6- one | Suzuki reaction using 3-bromo-5-fluoro-2- methoxy-pyridin-4-amine (Intermediate #400_1), ethyl 2-[2-(4,4,5,5-tetramethyl- 1,3,2-dioxaborolan-2- yl)phenyl]propanoate Intermediate N64_2, K₂CO₃, bis(di-tert-butyl(4- dimethylaminophenyl)phosphine)dichloro palladium(II) heated in dioxane/water at 90° C. followed by cyclization with K₂CO₃ in EtOH at 80° C. (56% yield). LC-MS m/z [M + H]⁺: 273.2; purity: 97%. ¹H NMR (400 |
| | | | MHz, DMSO-d₆) δ 10.30 (s, 1H), 8.19 (d, J = |
| | | | 1.5 Hz, 1H), 7.75 (dd, J = 8.2, 1.4 Hz, |
| | | | 1H), 7.49 (td, J = 7.5, 1.4 Hz, 1H), 7.43- |
| | | | 7.32 (m, 2H), 3.88 (s, 3H), 3.39 (q, J = 6.7 |
| | | | Hz, 1H), 1.45 (d, J = 6.8 Hz, 3H). |
| NN356 | 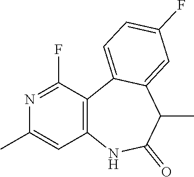 | 1,9-difluoro-3,7- dimethyl-5,7- dihydropyrido[4,3- d][3]benzazepin-6- one | Suzuki reaction using ethyl 2-(2-bromo-5- fluoro-phenyl)propanoate (Intermediate N70_2), 2-fluoro-6-methyl-3-(4,4,5,5- tetramethyl-1,3,2-dioxaborolan-2- yl)pyridin-4-amine (Intermediate N16_1), CsF, bis(di-tert-butyl(4- dimethylaminophenyl)phosphine)dichloro- palladium(II) heated in dioxane/water at 90° C. followed by cyclization with K₂CO₃ in EtOH at 80° C. (41% yield). LC-MS m/z [M + H]⁺: 275.2; purity: 95%. ¹H NMR (400 |
| | | | MHz, DMSO-d₆) δ 10.67 (s, 1H), 7.72- |
| | | | 7.65 (m, 1H), 7.30-7.18 (m, 2H), 6.95 (s, |
| | | | 1H), 3.44 (q, J = 6.8 Hz, 1H), 2.43 (s, 3H), |
| | | | 1.44 (d, J = 6.8 Hz, 3H). |
| NN357 | 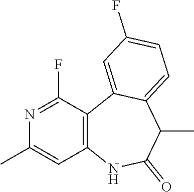 | 1,10-difluoro-3,7- dimethyl-5,7- dihydropyrido[3,4- a][3]benzazepin-6- one | Suzuki reaction using ethyl 2-(2-bromo-4- fluoro-phenyl)propanoate (Intermediate NN294), 2-fluoro-6-methyl-3-(4,4,5,5- tetramethyl-1,3,2-dioxaborolan-2- yl)pyridin-4-amine Intermediate N16_1, CsF, bis(di-tert-butyl(4- dimethylaminophenyl)phosphine)dichloro palladium(II) heated in dioxane/water at 80° C. followed by cyclization with K₂CO₃ in EtOH at 80° C. (50% yield). LC-MS m/z [M + H]⁺: 275.2; purity: 88%. ¹H NMR (400 MHz, DMSO-d₆) δ 10.67 (s, 1H), 7.48 |
| | | | (ddd, J = 10.2, 4.4, 2.7 Hz, 1H), 7.42 (dd, |
| | | | J = 8.8, 5.8 Hz, 1H), 7.36 (td, J = 8.5, 2.7 |
| | | | Hz, 1H), 6.96 (s, 1H), 3.40 (d, J = 6.8 Hz, |
| | | | 1H), 2.44 (s, 3H), 1.45 (d, J = 6.8 Hz, 3H). |
| NN358 | 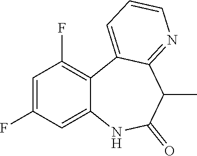 [SMILES: CC1C(=O)Nc2cc(F)cc(F)c2-c2cccnc21] | 9,11-difluoro-5- methyl-5,7- dihydropyrido[2,3- d][1]benzazepin-6- one | Suzuki reaction using ethyl 2-[3-(4,4,5,5- tetramethyl-1,3,2-dioxaborolan-2-yl)-2- pyridyl]propanoate (Intermediate N31_1), 2-bromo-3,5-difluoro-aniline (CAS 500357-40-4), CsF, bis(di-tert-butyl(4- dimethylaminophenyl)phosphine)dichloro- palladium(II) heated in dioxane/water at 90° C. followed by cyclization with K₂CO₃ in EtOH at 80° C. (63% yield). ¹H NMR (400 MHz, DMSO-d₆) δ 10.48 (s, 1H), 8.65 (dd, |
| | | | J = 4.7, 1.7 Hz, 1H), 8.03 (ddd, J = 7.9, 4.6, |
| | | | 1.7 Hz, 1H), 7.45 (dd, J = 7.9, 4.7 Hz, 1H), |
| | | | 7.29 (ddd, J = 11.4, 9.0, 2.6 Hz, 1H), 6.94 |
| | | | (ddd, J = 10.0, 2.7, 1.6 Hz, 1H), 3.57 (q, J = |
| | | | 6.6 Hz, 1H), 1.48 (d, J = 6.6 Hz, 3H); |
| | | | purity: 93%. |
| NN359 | 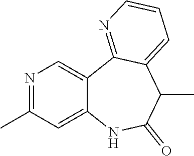 | 8,13-dimethyl- 3,10,14- triazatricyclo[9.4.0.0^{2,7}] pentadeca- 1(15),2,4,6,11,13- hexaen-9-one | Suzuki reaction using methyl 2-(2-chloro- 3-pyridyl)propanoate (CAS 1668475-78- 2), 2-methyl-5-(4,4,5,5-tetramethyl-1,3,2- dioxaborolan-2-yl)pyridin-4-amine (CAS 1668475-78-2), CsF, bis(di-tert-butyl(4- dimethylaminophenyl)phosphine)dichloro- palladium(II) heated in dioxane/water at 80° C. (30% yield). ¹H NMR (400 MHz, DMSO-d₆) δ 10.58 (s, 1H), 8.99 (s, 1H), 8.67 (dd, J = 4.7, 1.6 Hz, 1H), 7.81 (dt, J = |
| | | | 8.0, 1.2 Hz, 1H), 7.52 (dd, J = 7.9, 4.7 Hz, |
| | | | 1H), 6.97 (s, 1H), 3.40 (s, 1H), 2.50 (s, |
| | | | 3H), 1.46 (s, 3H). Purity: 96%. |
| NN360 | 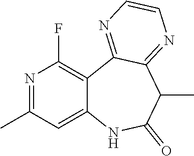 | 15-fluoro-8,13- dimethyl- 3,6,10,14- tetrazatricyclo[9.4.0.0^{2,7}] pentadeca- 1(15),2,4,6,11,13- hexaen-9-one | Suzuki reaction using ethyl 2-(3- chloropyrazin-2-yl)propanoate (Intermediate NN287), 2-fluoro-6-methyl- 3-(4,4,5,5-tetramethyl-1,3,2-dioxaborolan- 2-yl)pyridin-4-amine N16_1, CsF, bis(di- tert-butyl(4- dimethylaminophenyl)phosphine)dichloro- palladium(II) heated in dioxane/water at 80° C. followed by cyclization with K₂CO₃ in EtOH at 80° C. (31% yield). ¹H NMR (400 |
| | | | MHz, DMSO-d₆) δ 10.96 (s, 1H), 8.73 (s, |
| | | | 2H), 7.00 (s, 1H), 3.80 (d, J = 6.6 Hz, 1H), |
| | | | 2.46 (s, 3H), 1.49 (d, J = 6.7 Hz, 3H). |
| NN361 | 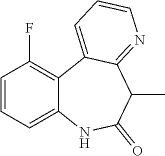 [SMILES: CC1C(=O)Nc2cccc(F)c2-c2cccnc21] | 11-fluoro-5- methyl-5,7- dihydropyrido[2,3- d][1]benzazepin-6- one | Suzuki reaction using 2-bromo-3-fluoro- aniline, ethyl 2-[3-(4,4,5,5-tetramethyl- 1,3,2-dioxaborolan-2-yl)-2- pyridyl]propanoate (Intermediate N31_1), CsF, bis(di-tert-butyl(4- dimethylaminophenyl)phosphine)dichloro- palladium(II) heated in dioxane/water at 90° C. followed by cyclization with K₂CO₃ in EtOH at 80° C. (41% yield). ¹H NMR (400 MHz, DMSO-d₆) δ 10.39 (s, 1H), 8.65 (dd, |
| | | | J = 4.7, 1.7 Hz, 1H), 8.04 (ddd, J = 7.9, 4.6, |
| | | | 1.7 Hz, 1H), 7.58-7.38 (m, 2H), 7.20 |
| | | | (ddd, J = 10.7, 8.3, 1.2 Hz, 1H), 7.11 (dt, J = |
| | | | 8.2, 1.0 Hz, 1H), 3.52 (q, J = 6.6 Hz, 1H), |
| | | | 1.48 (d, J = 6.7 Hz, 3H); purity: 82% |
| NN362 | 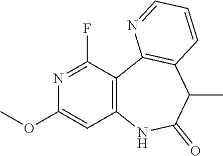 | 15-fluoro-13- methoxy-8-methyl- 3,10,14- triazatricyclo[9.4.0.0^{2,7}] pentadeca- 1(11),2(7),3,5,12,1 4-hexaen-9-one | Suzuki reaction using methyl 2-(2-chloro- 3-pyridyl)propanoate (CAS 2365492-24- 4), 2-fluoro-6-methoxy-3-(4,4,5,5- tetramethyl-1,3,2-dioxaborolan-2- yl)pyridin-4-amine (Intermediate N40_1), CsF, bis(di-tert-butyl(4- dimethylaminophenyl)phosphine)dichloro- palladium(II) heated in dioxane/water at 90° C. followed by cyclization with K₂CO₃ in EtOH at 80° C. (9% yield). LC-MS m/z |
| | | | [M + H]⁺: 274.2; purity: 53%. |
| NN363 | 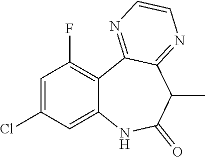 [SMILES: CC1C(=O)Nc2cc(Cl)cc(F)c2-c2nccnc21] | 9-chloro-11-fluoro- 5-methyl-5,7- dihydropyrazino[2,3- d][1]benzazepin- 6-one | Suzuki reaction using 5-chloro-3-fluoro-2- (4,4,5,5-tetramethyl-1,3,2-dioxaborolan-2- yl)aniline (Intermediate NN286), ethyl 2- (3-chloropyrazin-2-yl)propanoate (Intermediate NN287), CsF, SPhos Pd(crotyl)Cl heated in dioxane/water at 90° C. followed by cyclization with K₂CO₃ in EtOH at 80° C. (27% yield). LC-MS m/z [M + H]⁺: 278.0, 280.0; purity: 99%. |
| NN364 | 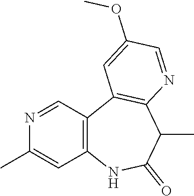 | 14-methoxy-5,10- dimethyl-4,8,12- triazatricyclo[9.4.0.0^{2,7}] pentadeca- 1(11),2(7),3,5,12,14- hexaen-9-one | Suzuki reaction using methyl 2-(3-bromo- 5-methoxy-2-pyridyl)propanoate (Intermediate NN297), 2-methyl-5- (4,4,5,5-tetramethyl-1,3,2-dioxaborolan-2- yl)pyridin-4-amine (CAS: 1668475-78-2), CsF, SPhos Pd(crotyl)Cl heated in dioxane/water at 90° C. followed by cyclization with K₂CO₃ in EtOH at 80° C. (27% yield). LC-MS m/z [M + H]⁺: 270.2; purity: 96%. ¹H NMR (400 MHz, DMSO-d₆) δ 8.62 (s, 1H), 8.31 (d, J = 2.7 Hz, 1H), 7.59 (d, J = 2.9 Hz, 1H), 6.81 (s, 1H), 3.89 (s, 3H), |
| | | | 3.59 (q, J = 7.0 Hz, 1H), 2.44 (s, 4H), 1.43 |
| | | | (d, J = 6.7 Hz, 3H). |
| NN365 | 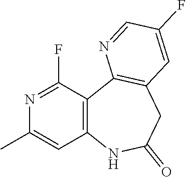 [SMILES: Cc1cc2c(c(F)n1)-c1ncc(F)cc1CC(=O)N2] | 5,15-difluoro-13- methyl-3,10,14- triazatricyclo[9.4.0.0^{2,7}] pentadeca- 1(11),2(7),3,5,12,14- hexaen-9-one | Suzuki reaction using methyl 2-(2-chloro- 5-fluoro-3-pyridyl)acetate (Intermediate NN289), 2-fluoro-6-methyl-3-(4,4,5,5- tetramethyl-1,3,2-dioxaborolan-2- yl)pyridin-4-amine (Intermediate N16_1), CsF, bis(di-tert-butyl(4- dimethylaminophenyl)phosphine)dichloro- palladium(II) heated in dioxane/water at 90° C. followed by cyclization with K₂CO₃ in EtOH at 80 (22% yield). LC-MS m/z [M + H]⁺: 262.1; purity: 46%. ¹H NMR (400 |
| | | | MHz, DMSO-d₆) δ 10.73 (s, 1H), 8.66 (d, J = |
| | | | 2.8 Hz, 1H), 7.95 (dd, J = 9.1, 2.8 Hz, |
| | | | 1H), 6.40 (s, 1H), 3.66-3.58 (m, 2H), 2.22 |
| | | | (s, 3H). |
| NN366 | 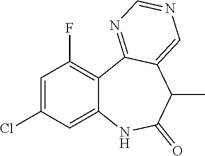 [SMILES: CC1C(=O)Nc2cc(Cl)cc(F)c2-c2ncncc21] | 9-chloro-11-fluoro- 5-methyl-5,7- dihydropyrimido[5,4- d][1]benzazepin- 6-one | Suzuki reaction using ethyl 2-(4- chloropyrimidin-5-yl)propanoate (Intermediate NN439), 5-chloro-3-fluoro- 2-(4,4,5,5-tetramethyl-1,3,2-dioxaborolan- 2-yl)aniline (Intermediate NN286), CsF, bis(di-tert-butyl(4- dimethylaminophenyl)phosphine)dichloro- palladium(II) heated in dioxane/water at 90° C. LC-MS [M + H]⁺: 278.1, 280.0; purity: 97%. ¹H NMR (400 MHz, DMSO-d₆) δ |
| | | | 10.57 (s, 1H), 9.23 (s, 1H), 8.88 (s, 1H), |
| | | | 7.45 (dd, J = 10.5, 2.0 Hz, 1H), 7.16 (t, J = |
| | | | 1.7 Hz, 1H), 3.57 (q, J = 6.8 Hz, 1H), 1.53 |
| | | | (d, J = 6.9 Hz, 3H). |
| NN367 | 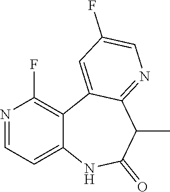 [SMILES: CC1C(=O)Nc2ccnc(F)c2-c2cc(F)cnc21] | 3,14-difluoro-10- methyl-4,8,12- triazatricyclo[9.4.0.0^{2,7}] pentadeca- 1(11),2,4,6,12,14- hexaen-9-one | Suzuki reaction using 3-bromo-2-fluoro- pyridin-4-amine (CAS 1364917-17-8), ethyl 2-[5-fluoro-3-(4,4,5,5-tetramethyl- 1,3,2-dioxaborolan-2-yl)-2- pyridyl]propanoate (Intermediate NN304), CsF, bis(di-tert-butyl(4- dimethylaminophenyl)phosphine)dichloro- palladium(II) heated in dioxane/water at 90° C. followed by cyclization with K₂CO₃ in EtOH at 80 (22% yield). LC-MS m/z [M + H]⁺: 262.1; purity: 75%. ¹H NMR (400 MHz, DMSO-d₆) δ 10.93 (s, 1H), 8.71 (d, J = |
| | | | 2.8 Hz, 1H), 8.21 (d, J = 5.5 Hz, 1H), |
| | | | 8.15-7.92 (m, 1H), 7.18 (d, J = 5.6 Hz, |
| | | | 1H), 3.69-3.62 (m, 1H), 1.49 (d, J = 6.6 |
| | | | Hz, 3H). |
| NN368 | 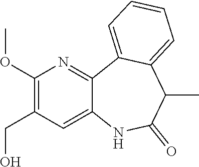 | 3-(hydroxymethyl)- 2-methoxy-7- methyl-5,7- dihydropyrido[3,2- d][3]benzazepin-6- one | Suzuki reaction using (5-amino-6-bromo- 2-methoxy-3-pyridyl)methanol (Intermediate NN247), ethyl 2-[2-(4,4,5,5- tetramethyl-1,3,2-dioxaborolan-2- yl)phenyl]propanoate (Intermediate N64_2), CsF, bis(di-tert-butyl(4- dimethylaminophenyl)phosphine)dichloro- palladium(II) heated in dioxane/water at 100° C. followed by cyclization with K₂CO₃ in EtOH at 90° C. (2% yield). LC-MS m/z |
| | | | [M + H]⁺: 285.2; purity: 86%. |
| NN369 | 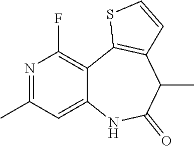 | 14-fluoro-7,12- dimethyl-3-thia- 9,13- diazatricyclo[8.4.0.0^{2,6}] tetradeca- 1(14),2(6),4,10,12- pentaen-8-one | Suzuki reaction using methyl 2-(2-bromo- 3-thienyl)propanoate (Intermediate NN302), 2-fluoro-6-methyl-3-(4,4,5,5- tetramethyl-1,3,2-dioxaborolan-2- yl)pyridin-4-amine (Intermediate N16_1), K₃PO₄, SPhos Pd(crotyl)Cl heated in toluene at 100° C. followed by cyclization with LiHMDS in THF at 0° C. (33% yield). LC-MS m/z [M + H]⁺: 263.2; purity: 80%. ¹H |
| | | | NMR (400 MHz, DMSO-d₆) δ 10.76 (s, |
| | | | 1H), 7.82 (d, J = 5.2 Hz, 1H), 7.09 (d, J = |
| | | | 5.3 Hz, 1H), 6.97 (s, 1H), 3.94 (q, J = 7.2 |
| | | | Hz, 1H), 2.42 (s, 3H), 1.46 (d, J = 7.0 Hz, |
| | | | 3H). |
| NN370 | 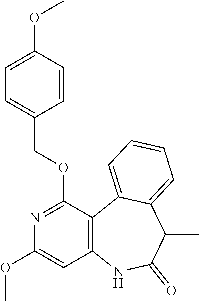 [SMILES: COc1ccc(COc2nc(OC)cc3c2-c2ccccc2C(C)C(=O)N3)cc1] | 3-methoxy-1-[(4- methoxyphenyl)meth- oxy]-7-methyl- 5,7- dihydropyrido[3,4- a][3]benzazepin-6- one | Suzuki reaction using 3-bromo-6- methoxy-2-[(4- methoxyphenyl)methoxy]pyridin-4-amine (Intermediate NN264), ethyl 2-[2-(4,4,5,5- tetramethyl-1,3,2-dioxaborolan-2- yl)phenyl]propanoate (Intermediate N64_2), CsF, bis(di-tert-butyl(4- dimethylaminophenyl)phosphine)dichloro- palladium(II) heated in dioxane/water at 90° C. followed by cyclization with K₂CO₃ in EtOH at 80° C. (58% yield). LC-MS m/z [M + H]⁺: 391.2; purity: 98%. ¹H NMR (400 MHz, DMSO-d₆) δ 10.22 (s, 1H), 7.69 (dd, J = 7.9, 1.5 Hz, 1H), 7.45-7.32 (m, 3H), 7.31-7.22 (m, 2H), 6.95-6.88 (m, 2H), 6.19 (s, 1H), 5.46 (d, J = 12.1 Hz, 1H), 5.26 (d, J = 12.0 Hz, 1H), 3.92 (s, 3H), 3.74 (s, 3H), 3.35-3.27 (m, 1H), 1.42 (d, J = 6.8 Hz, 3H). |
| NN371 | 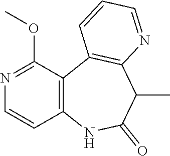 [SMILES: COc1nccc2c1-c1cccnc1C(C)C(=O)N2] | 3-methoxy-10- methyl-4,8,12- triazatricyclo[9.4.0.0^{2,7}] pentadeca- 1(11),2(7),3,5,12,14- hexaen-9-one | Suzuki reaction using 3-bromo-2- methoxy-pyridin-4-amine (CAS No: 215364-86-6), ethyl 2-[3-(4,4,5,5- tetramethyl-1,3,2-dioxaborolan-2-yl)-2- pyridyl]propanoate (Intermediate N31_1), CsF, SPhos Pd(crotyl)Cl heated in dioxane/water at 90° C. followed by cyclization with K₂CO₃ in EtOH at 90° C. (52% yield). LC-MS m/z [M + H]⁺: 256.2; purity: 100%. ¹H NMR (400 MHz, DMSO-d₆) δ |
| | | | 10.53 (s, 1H), 8.59 (dd, J = 4.7, 1.7 Hz, |
| | | | 1H), 8.14 (dd, J = 8.0, 1.7 Hz, 1H), 8.11 (d, |
| | | | J = 5.6 Hz, 1H), 7.39 (dd, J = 7.9, 4.7 Hz, |
| | | | 1H), 6.86 (d, J = 5.6 Hz, 1H), 3.91 (s, 3H), |
| | | | 3.48 (q, J = 6.6 Hz, 1H), 1.47 (d, J = 6.7 |
| | | | Hz, 3H). |
| NN372 | 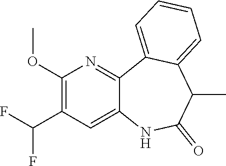 [SMILES: COc1nc2c(cc1C(F)F)NC(=O)C(C)c1ccccc1-2] | 3-(difluoromethyl)- 2-methoxy-7- methyl-5,7- dihydropyrido[3,2- d][3]benzazepin-6- one | Suzuki reaction using 2-bromo-5- (difluoromethyl)-6-methoxy-pyridin-3- amine (Intermediate NN261), ethyl 2-[2- (4,4,5,5-tetramethyl-1,3,2-dioxaborolan-2- yl)phenyl]propanoate (Intermediate N64_2), CsF, bis(di-tert-butyl(4- dimethylaminophenyl)phosphine)dichloro- palladium(II) heated in dioxane/water at 90° C. followed by cyclization with K₂CO₃ in EtOH at 80° C. (73% yield). LC-MS m/z [M + H]⁺: 305.1; purity: 97%. |
| NN373 | 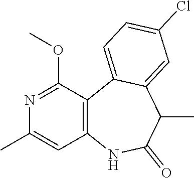 | 9-chloro-1- methoxy-3,7- dimethyl-5,7- dihydropyrido[3,4- a][3]benzazepin-6- one | Suzuki reaction using 3-bromo-2- methoxy-6-methyl-pyridin-4-amine (Intermediate NN274), methyl 2-[5-chloro- 2-(4,4,5,5-tetramethyl-1,3,2-dioxaborolan- 2-yl)phenyl]propanoate (Intermediate N73_2), CsF, bis(di-tert-butyl(4- dimethylaminophenyl)phosphine)dichloro- palladium(II) heated in dioxane/water at 90° C. followed by cyclization with K₂CO₃ in EtOH at 80° C. (12% yield). LC-MS m/z [M + H]⁺: 303.1, 305.1; purity: 72%. |
| NN374 | 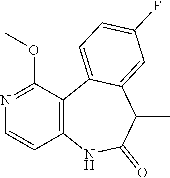 [SMILES: COc1nccc2c1-c1ccc(F)cc1C(C)C(=O)N2] | 9-fluoro-1- methoxy-7-methyl- 5,7- dihydropyrido[4,3- d][3]benzazepin-6- one | Suzuki reaction using ethyl 2-[5-fluoro-2- (4,4,5,5-tetramethyl-1,3,2-dioxaborolan-2- yl)phenyl]propanoate (Intermediate N70_3), 3-bromo-2-methoxy-pyridin-4- amine (CAS No: 215364-86-6), CsF, SPhos Pd(crotyl)Cl heated in dioxane/ water at 90° C. followed by cyclization with K₂CO₃ in EtOH at 80° C. (18% yield). LC- MS m/z [M + H]⁺: 273.2; purity: 100%. ¹H NMR (400 MHz, DMSO-d₆) δ 10.40 (s, 1H), 8.07 (d, J = 5.5 Hz, 1H), 7.76 (dd, J = |
| | | | 8.7, 6.0 Hz, 1H), 7.25-7.07 (m, 2H), 6.81 |
| | | | (d, J = 5.6 Hz, 1H), 3.89 (s, 3H), 3.35- |
| | | | 3.27 (m, 1H), 1.42 (d, J = 6.8 Hz, 3H). |
| NN375 | 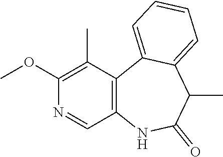 | 2-methoxy-1,7- dimethyl-5,7- dihydropyrido[4,3- a][3]benzazepin-6- one | Suzuki reaction using 4-bromo-6- methoxy-5-methyl-pyridin-3-amine (Intermediate NN252), ethyl 2-[2-(4,4,5,5- tetramethyl-1,3,2-dioxaborolan-2- yl)phenyl]propanoate (Intermediate N64_2), CsF, SPhos Pd(crotyl)Cl heated in dioxane/water at 100° C. followed by cyclization with K₂CO₃ in EtOH at 80° C. (14% yield). LC-MS m/z [M + H]⁺: 269.2; purity: 93%. ¹H NMR (400 MHz, DMSO-d₆) δ 9.84 |
| | | | (s, 1H), 7.92 (s, 1H), 7.52 (m, 2H), 7.41- |
| | | | 7.35 (m, 2H), 3.92 (s, 3H), 3.35 (q, J = 6.8 |
| | | | Hz, 1H), 2.19 (s, 3H), 1.41 (d, J = 6.8 Hz, |
| | | | 3H). |
| NN376 | 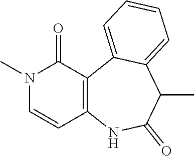 | 2,7-dimethyl-5,7- dihydro-1H- benzo[d]pyrido[4,3- b]azepine- 1,6(2H)-dione | Suzuki reaction using 4-amino-3-bromo-1- methylpyridin-2(1H)-one (Intermediate NN273), ethyl 2-[2-(4,4,5,5-tetramethyl- 1,3,2-dioxaborolan-2- yl)phenyl]propanoate (Intermediate N64_2), K₂CO₃, bis(di-tert-butyl(4- dimethylaminophenyl)phosphine)dichloro- palladium(II) heated in dioxane/water at 90° C. followed by cyclization with K₂CO₃ in EtOH at 90° C. (5 % yield). ¹H NMR (400 |
| | | | MHz, CDCl₃) δ 8.59 (s, 1H), 7.96 (dd, J = |
| | | | 8.2, 1.5 Hz, 1H), 7.43-7.35 (m, 1H), 7.34- |
| | | | 7.28 (m, 2H), 7.24 (d, J = 7.3 Hz, 1H), |
| | | | 6.05 (d, J = 7.3 Hz, 1H), 3.60 (s, 3H), 3.30 |
| | | | (q, J = 6.9 Hz, 1H), 1.58 (d, J = 6.9 Hz, 3H); |
| | | | purity: 75%. |
| NN377 | 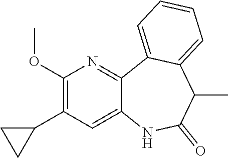 [SMILES: COc1nc2c(cc1C1CC1)NC(=O)C(C)c1ccccc1-2] | 3-cyclopropyl-2- methoxy-7-methyl- 5,7- dihydropyrido[2,3- a][3]benzazepin-6- one | Suzuki reaction using 2-bromo-5- cyclopropyl-6-methoxy-pyridin-3-amine NN440, ethyl 2-[2-(4,4,5,5-tetramethyl- 1,3,2-dioxaborolan-2- yl)phenyl]propanoate(Intermediate N64_2), CsF, bis(di-tert-butyl(4- dimethylaminophenyl)phosphine)dichloro- palladium(II) heated in dioxane/water at 90° C. (41% yield). LC-MS m/z [M + H]⁺: 295.2; purity: 97%. |
| NN378 | 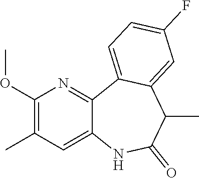 | 9-fluoro-2- methoxy-3,7- dimethyl-5,7- dihydropyrido[3,2- d][3]benzazepin-6- one | Suzuki reaction using 2-bromo-6- methoxy-5-methyl-pyridin-3-amine (Intermediate NN285), ethyl 2-[5-fluoro-2- (4,4,5,5-tetramethyl-1,3,2-dioxaborolan-2- yl)phenyl]propanoate(Intermediate N70_3), CsF, bis(di-tert-butyl(4- dimethylaminophenyl)phosphine)dichloro- palladium(II) heated in dioxane/water at 90° C. (89% yield). LC-MS m/z [M + H]⁺: 287.2; purity: 86%. ¹H NMR (400 MHz, DMSO-d₆) δ 10.02 (s, 1H), 8.02-7.91 (m, |
| | | | 1H), 7.34 (s, 1H), 7.29 (td, J = 8.6, 2.7 Hz, |
| | | | 1H), 7.15 (dd, J = 10.3, 2.6 Hz, 1H), 3.96 |
| | | | (s, 3H), 3.28 (q, J = 6.9 Hz, 1H), 2.20 (s, |
| | | | 3H), 1.44 (d, J = 6.9 Hz, 3H). |
| NN234 | 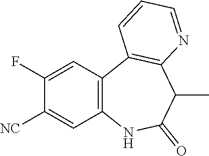 [SMILES: CC1C(=O)Nc2cc(C#N)c(F)cc2-c2cccnc21] | 10-fluoro-5- methyl-6-oxo-5,7- dihydropyrido[2,3- d][1]benzazepine- 9-carbonitrile | Suzuki reaction using 5-amino-4-bromo-2- fluorobenzonitrile, ethyl 2-[3-(4,4,5,5- tetramethyl-1,3,2-dioxaborolan-2-yl)-2- pyridyl]propanoate (Intermediate N31_1), K₂CO₃, Pd(dppf)Cl₂ heated in dioxane/ water at 90° C. followed by cyclization with LiHMDS in THF at 0° C. |
| NN444 | 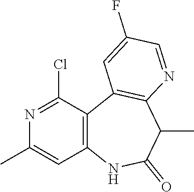 | 3-chloro-14-fluoro- 5,10-dimethyl- 4,8,12- triazatricyclo[9.4.0.0^{2,7}] pentadeca- 1(11),2(7),3,5,12,14- hexaen-9-one | From 3-bromo-2-chloro-6-methyl-pyridin- 4-amine N30_1 and ethyl 2-[5-fluoro-3- (4,4,5,5-tetramethyl-1,3,2-dioxaborolan-2- yl)-2-pyridyl]propanoate NN304, CsF, bis(di-tert-butyl(4- dimethylaminophenyl)phosphine)dichloro- palladium(II) heated in dioxane/water at 85° C. followed by cyclization with LiHMDS in toluene at RT (yield 30%). LC-MS m/z: [M + H]⁺: 292; purity: 76%. ¹H NMR (400 MHz, DMSO-d₆) δ 10.74 (s, 1H), 8.68 (d, J = 2.8 Hz, 1H), 8.19 (dd, J = 9.9, 2.8 Hz, |
| | | | 1H), 7.06 (s, 1H), 3.67 (q, J = 6.6 Hz, 1H), |
| | | | 2.49 (s, 3H), 1.46 (d, J = 6.6 Hz, 3H). ¹⁹F |
| | | | NMR (376 MHz, DMSO) δ −130.63 (d, J = |
| | | | 9.9 Hz). |

### Intermediate NN379: 14-methoxy-7,12-dimethyl-5,6,9,13-tetrazatricyclo[8.4.0.0^{2,7}]tetradeca-1(14),2,4,10,12-pentaen-8-one

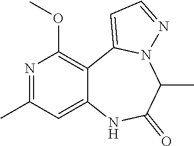

### Step 1: Synthesis of 2-methoxy-6-methyl-3-(2-tetrahydropyran-2-ylpyrazol-3-yl)pyridin-4-amine (NN380)

Nitrogen was sparged through a solution of 3-bromo-2-methoxy-6-methyl-pyridin-4-amine Intermediate NN274 (1.51 g, 6.96 mmol), 1-tetrahydropyran-2-yl-5-(4,4,5,5-tetramethyl-1,3,2-dioxaborolan-2-yl)pyrazole (CAS 903550-26-5) (2.52 g, 9.04 mmol), K₂CO₃ (2.88 g, 21 mmol) and bis(di-tert-butyl(4-dimethylaminophenyl)phosphine)dichloropalladium(II) (197 mg, 0.28 mmol) in 1,4-dioxane (59 mL) and water (1.7 mL). The reaction mixture was heated under reflux for 2.5 h, then cooled to RT. The solution was filtered on a pad of celite and washed with EtOAc (200 mL). The filtrate was concentrated, and the crude was purified by column chromatography on silica gel (using a gradient of 0-100% EtOAc in isohexane as eluent) to afford the title compound as a brown gum (1.9 g, yield: 83%). LC-MS m/z [M+H]⁺: 289.2; purity: 96%.

Step-2: Synthesis of 2-chloro-N-[2-methoxy-6-methyl-3-(2-tetrahydropyran-2-ylpyrazol-3-yl)-4-pyridyl]propanamide (NN381)

To a solution of 2-methoxy-6-methyl-3-(2-tetrahydropyran-2-ylpyrazol-3-yl)pyridin-4-amine NN380 (1.92 g, 5.99 mmol) in DMF (59 mL) at 0° C. was added NaH (60%, 288 mg, 7.2 mmol). The solution was stirred for 15 min at RT then 2-chloropropanoyl chloride (0.85 mL, 8.4 mmol) was added dropwise. The reaction mixture was stirred for 1 h at 0° C. then warmed to RT and stirred for 1 h. Water (120 mL) was added to the reaction mixture which was then extracted with EtOAc (3×80 mL). The combined organic extracts were washed with brine (150 mL), dried over Na₂SO₄, filtered and concentrated under reduced pressure. The crude was purified by column chromatography on silica gel (using a gradient of 0-100% EtOAc in isohexane as eluent) to afford the title compound as a brown wax (1.35 g, yield: 55%). LC-MS m/z [M+H]⁺: 379.2, 381.2; purity: 93% (as a mixture of diastereoisomers).

### Step-3: Synthesis of 2-chloro-N-[2-methoxy-6-methyl-3-(1H-pyrazol-5-yl)-4-pyridyl]propanamide (NN382)

To a suspension of 2-chloro-N-[2-methoxy-6-methyl-3-(2-tetrahydropyran-2-ylpyrazol-3-yl)-4-pyridyl]propanamide NN381 (1.35 g, 3.31 mmol) was added a solution of 4M HCl in 1,4-dioxane (8.28 mL, 33 mmol) in 1,4-dioxane (8.27 mL) was stirred at RT overnight. Volatiles were removed under reduced pressure and the residue was partitioned between EtOAc (80 mL) and a saturated aqueous solution of NaHCO₃ (100 mL), phases were separated. The aqueous layer was extracted with EtOAc-MeOH 95:5 (2×80 mL). The combined organic extracts were washed with water (50 mL), brine (100 mL), dried over Na₂SO₄, filtered and concentrated under reduced pressure. The solid was triturated with TBME and filtered, the filtrate was then concentrated to dryness. The crude was purified by column chromatography on silica gel (using a gradient of 0-80% EtOAc in isohexane as eluent) to afford the title compound as a yellow gum (550 mg, yield: 35%). LC-MS m/z [M+H]⁺: 295.2, 297.2; purity: 71%. ¹H NMR (400 MHz, DMSO-d₆) δ 13.35 (s, 1H), 12.66 (d, J=7.5 Hz, 1H), 8.03 (d, J=4.1 Hz, 1H), 7.91 (t, J=2.1 Hz, 1H), 6.87 (t, J=2.1 Hz, 1H), 4.80 (q, J=6.9 Hz, 1H), 3.94 (d, J=1.2 Hz, 3H), 2.40 (d, J=1.6 Hz, 3H), 1.69 (d, J=6.9 Hz, 3H); purity: 65%.

### Step 4 Synthesis of 14-methoxy-7,12-dimethyl-5,6,9,13-tetrazatricyclo[8.4.0.0^{2,6}]tetradeca-1(14),2,4,10,12-pentaen-8-one (NN379)

To a solution of 2-chloro-N-[2-methoxy-6-methyl-3-(1H-pyrazol-5-yl)-4-pyridyl]propanamide NN382 (65%, 550 mg, 1.21 mmol) in DMF (18.0 mL) was added NaH (60% purity, 102 mg, 2.55 mmol). The reaction mixture was stirred at rt for 3 h then water (40 mL) was added followed by AcOH (0.5 mL). The resulting suspension was extracted with EtOAc (3×30 mL). The combined organic extracts were washed with brine (50 mL), dried over Na₂SO₄, filtered and concentrated under reduced pressure. The crude was purified by column chromatography on silica gel (using a gradient of 0-10% MeOH in DCM as eluent) to afford the title compound as an off-white solid (306 mg, yield: 64%). LC-MS m/z [M+H]⁺: 259.2; purity: 65%.

### Intermediate NN181: 9-methoxy-5-methyl-5,7-dihydropyrido[2,3-d][1]benzazepin-6-one

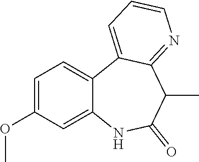 [SMILES: COc1ccc2c(c1)NC(=O)C(C)c1ncccc1-2]

Under inert atmosphere, 9-bromo-5-methyl-5,7-dihydropyrido[2,3-d][1]benzazepin-6-one NN227 (15.2 mg, 0,050 mmol), tBuBrettPhos Pd G₃ (4.27 mg, 0,005 mmol), tBuBrettPhos (2.42 mg, 0,005 mmol) and sodium tert-butoxide (14.4 mg, 0,150 mmol) were suspended in 1,4-dioxane (0.5 mL) and methanol (10 μL, 0.247 mmol). The reaction mixture was stirred at room temperature for 18 h and then diluted with water and extracted three times with EtOAc. The combined organic extracts were dried over Na₂SO₄, filtered and concentrated to dryness. The crude was purified by reverse phase HPLC (Purification Method P_B) to afford the title compound (2 mg, yield:15%). LC-MS m/z [M+H]⁺: 255.0; purity: 99%.

### Intermediate NN182: 5-methyl-6-oxo-5,7-dihydropyrido[2,3-d][1]benzazepine-9-carbonitrile

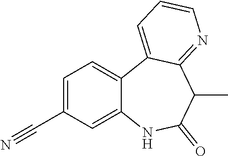 [SMILES: CC1C(=O)Nc2cc(C#N)ccc2-c2cccnc21]

A mixture of 9-bromo-5-methyl-5,7-dihydropyrido[2,3-d][1]benzazepin-6-one NN227 (0.20 mmol), tetrakis(triphenylphosphine)palladium(0) (23 mg, 0,020 mmol) and zinc cyanide (48 mg, 0,400 mmol) in degassed N,N-dimethylacetamide (1 mL) was stirred and heated at 150° C. for 20 min under inert atmosphere. The reaction mixture was neutralized with a saturated NaHCO₃ solution, diluted with AcOEt and filtered through a pad of Celite®. The filtrate was extracted three times with EtOAc. The combined organic extracts were washed with brine, dried over Na₂SO₄, filtered and concentrated to dryness. The crude was purified by reverse phase HPLC (Purification Method P_B) to afford the title compound (5 mg, yield: 10%). LC-MS m/z [M+H]⁺: 250.0; purity: 100%. The pad of Celite® was further washed with water (3 ×), the aqueous phase was discarded and then washed with N,N-dimethylacetamide (3 ×). The filtrate was concentrated under vacuum to afford the title compound (33 mg, yield: 66%). LC-MS m/z [M+H]⁺: 250.1; purity: 97%.

### Intermediate NN183: 2-(9-bromo-5-methyl-6-oxo-5H-pyrido[2,3-d][1]benzazepin-7-yl)-N-[4-(difluoromethyl)phenyl]acetamide

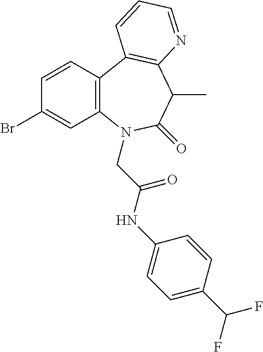 [SMILES: CC1C(=O)N(CC(=O)Nc2ccc(C(F)F)cc2)c2cc(Br)ccc2-c2cccnc21]

9-bromo-5-methyl-5,7-dihydropyrido[2,3-d][1]benzazepin-6-one NN227 (1 g, 3,298 mmol), 2-chloro-N-[4-(difluoromethyl)phenyl]acetamide (797 mg, 3.63 mmol), potassium iodide (274 mg, 1.65 mmol), and potassium carbonate (912 mg, 6.60 mmol) were suspended in N,N-dimethylacetamide (10 mL). The reaction mixture was stirred at 60° C. for 18h. To complete the reaction, another addition of 2-chloro-N-[4-(difluoromethyl)phenyl]acetamide (145 mg, 0.66 mmol) was added at room temperature and then heated at 60° C. for 5 days. The reaction mixture was quenched with water and the resulting precipitate was filtered, washed with water (3×) and dried under vacuo to afford the title compound as pale yellow solid (1.25 g, yield: 71%). LC-MS m/z [M+H]⁺: 486/488; purity: 91%.

### Intermediate NN187: 2-(9-bromo-5-methyl-6-oxo-5H-pyrido[2,3-d][1]benzazepin-7-yl)-N-(4-chloro-3-fluoro-phenyl)acetamide

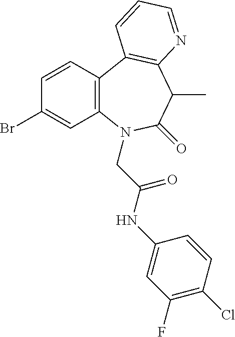 [SMILES: CC1C(=O)N(CC(=O)Nc2ccc(Cl)c(F)c2)c2cc(Br)ccc2-c2cccnc21]

9-bromo-5-methyl-5,7-dihydropyrido[2,3-d][1]benzazepin-6-one NN227 (1.5 g, 4.9 mmol), 2-chloro-N-(4-chloro-3-fluoro-phenyl)acetamide (1,2 g, 5.4 mmol), potassium iodide (410 mg, 2.5 mmol) and potassium carbonate (1,4 g, 10 mmol) were suspended in N,N-dimethylacetamide (15 mL). The reaction mixture was stirred at 50° C. for 2 days. The reaction mixture was quenched with water and the resulting precipitate was filtered, washed with water (3×) and dried under vacuo to afford the title compound as beige solid which was taken crude to the next step (1.866 g, yield: 68%). LC-MS m/z [M+H]⁺: 488/490; purity: 88%.

### Intermediate NN185: 5-methyl-6-oxo-5,7-dihydropyrido[2,3-d][1]benzazepine-9-carbaldehyde

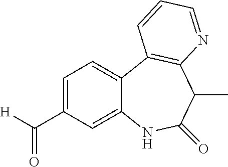 [SMILES: CC1C(=O)Nc2cc(C=O)ccc2-c2cccnc21]

To a solution of 9-bromo-5-methyl-5,7-dihydropyrido[2,3-d][1]benzazepin-6-one NN227 (0.5 g; 1.649 mmol) in toluene (15 mL) was added hexakis(μ-acetato)tripalladium(II) (111.1 mg; 0.165 mmol), butyldi-1-adamantylphosphine (186.7 mg; 0.495 mmol) and N,N,N′,N′-tetramethyl ethylenediamine (0.497 mL). The reaction mixture was placed in a stainless steel reactor at 100° C. and 5 Bars of syngas (mixture hydrogen and carbon monoxide 50/50) for 4 h. The reaction was not complete (50% conversion), so additional hexakis(p-acetato)tripalladium(II) (111.1 mg; 0.165 mmol) and butyldi-1-adamantylphosphine (186.7 mg; 0.495 mmol) were added and the reaction was pursued at 100° C. under 5 Bars of syngas for 16 h. Additional hexakis(p-acetato)tripalladium(II) (111.1 mg; 0.165 mmol) and butyldi-1-adamantylphosphine (186.7 mg; 0.495 mmol) were added again and the reaction was further pursued at 100° C. under 5 Bars of syngas for an additional 5 h. The reaction mixture was cooled down to RT and was passed through a pad of diatomaceous earth and the pad was thoroughly washed with toluene. The filtrate was concentrated under vacuum at 40° C. The crude obtained after evaporation is purified by flash chromatography (silica, gradient using dichloromethane and ethyl acetate as eluent) afforded the title product (94 mg, yield: 21%). LCMS: [M+H]⁺ m/z 253.

### Intermediate NN188: N-(4-chloro-3-fluoro-phenyl)-2-(9-formyl-5-methyl-6-oxo-5H-pyrido[2,3-d][1]benzazepin-7-yl)acetamide

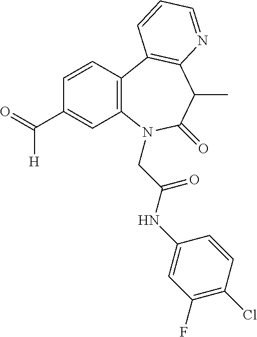 [SMILES: CC1C(=O)N(CC(=O)Nc2ccc(Cl)c(F)c2)c2cc(C=O)ccc2-c2cccnc21]

2-(9-bromo-5-methyl-6-oxo-5H-pyrido[2,3-d][1]benzazepin-7-yl)-N-(4-chloro-3-fluoro-phenyl)acetamide NN187 (500 mg, 0,900 mmol), Hexakis(p-acetato)tripalladium(II) (0,2 equiv., 0,180 mmol), butyldi-1-adamantylphosphine (0,6 equiv., 0,540 mmol) and N,N,N′,N′-tetramethylethylenediamine (2 equiv., 1,801 mmol,) was suspended in Toluene (10 mL). The reaction mixture was stirred under 5 bars of syngas at 100° C. for 16h. To complete the reaction, another addition of butyldi-1-adamantylphosphine (102 mg, 0,2703 mmol, 95 mass %) and Hexakis(p-acetato)tripalladium(II) (60 mg, 0,0891 mmol, 100 mass %) was added at room temperature and then stirred at 100° C. for 2 days. The reaction mixture was filtered through a pad of Celite® and rinsed with EtOAc (3×). The filtrate was concentrated to dryness and purified by flash chromatography on silica gel (using a gradient of 10% to 70% AcOEt in Heptane) to afford the title compound (52 mg, yield: 10%). LC-MS m/z [M+H]⁺: 438/440; purity: 90%. The pad of Celite® was further washed with DMF (3×). The filtrate was concentrated under vacuum to afford the title compound as white solid (232 mg, yield: 50%). LC-MS m/z [M+H]⁺: 438/440; purity: 97%.

### Intermediate NN184: 9-(3,3-difluoroazetidin-1-yl)-5-methyl-5,7-dihydropyrido[2,3-d][1]benzazepin-6-one

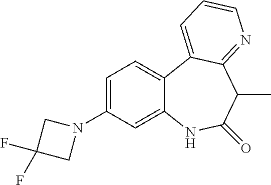 [SMILES: CC1C(=O)Nc2cc(N3CC(F)(F)C3)ccc2-c2cccnc21]

Under inert atmosphere, 9-bromo-5-methyl-5,7-dihydropyrido[2,3-d][1]benzazepin-6-one NN227 (303 mg, 1 mmol) and 3,3-difluoroazetidine hydrochloride (204 mg, 1.5 mmol) were suspended in dry 1,4-dioxane (5 mL). The solution was added into a vial containing Ruphos Pd G₃ (84 mg, 0.10 mmol) and Ruphos (47 mg, 0.10 mmol). The vial was sonicated a few seconds and transferred into a vial containing sodium tert-butoxyde (288 mg, 3.0 mmol). The reaction mixture was stirred and heated at 90° C. overnight under inert atmosphere. The reaction mixture was filtered through a pad of Celite® and washed with AcOEt (2×). The filtrate was diluted with water and extracted with EtOAc (2×). The combined organic extracts were washed with brine, dried over MgSO₄, filtered and concentrated to dryness. To the crude was added DCM and stirred for 2 min. The resulting precipitate was collected by filtration and dried under vacuum to afford the title compound as a white solid (205 mg, yield: 65%). LC-MS m/z [M+H]⁺: 316.0; purity: 92%.

### Intermediate NN186: 9-(difluoromethyl)-5-methyl-5,7-dihydropyrido[2,3-d][1]benzazepin-6-one

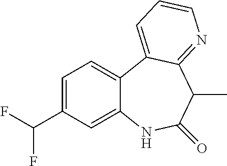 [SMILES: CC1C(=O)Nc2cc(C(F)F)ccc2-c2cccnc21]

At −78° C., to a suspension of 5-methyl-6-oxo-5,7-dihydropyrido[2,3-d][1]benzazepine-9-carbaldehyde NN185 (16 mg, 0,063 mmol) in dry DCM (1 mL) and under inert atmosphere was added diethylaminosulfur trifluoride (13 μL, 0.095 mmol). The reaction mixture was then allowed to warm to room temperature and stirred for 30 min. After this time, a second addition of diethylaminosulfur trifluoride (13 μL, 0.095 mmol) was added at 0° C. and stirred at room temperature overnight. To complete the reaction, a third addition of diethylaminosulfur trifluoride (26 μL, 0.19 mmol) was added at 0° C. and stirred at room temperature for 2 h. The reaction mixture was quenched with saturated NaHCO₃ solution and extracted with EtOAc (3×). The combined organic extracts were washed with brine, dried over Na₂SO₄, filtered and concentrated to dryness to afford the title compound which was taken crude to the next step (11.4 mg, yield: 45%). LC-MS m/z [M+H]⁺: 275.1; purity: 69%.

### Intermediate NN192: 3,5-dimethoxy-10-methyl-4,8,12-triazatricyclo[9.4.0.0^{2,7}]pentadeca-1(11),2(7),3,5,12,14-hexaen-9-one

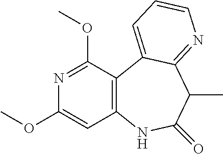

In a sealed tube, to a suspension of 3,5-difluoro-10-methyl-4,8,12-triazatricyclo[9.4.0.0^{2,7}]pentadeca-1(15),2(7),3,5,11,13-hexaen-9-one (Intermediate NN229, 1.7 g, 6.5 mmol) in MeOH (20 mL) was added K₂CO₃ (2.8 g, 20 mmol) and the reaction mixture heated at 70° C. for 24 h and at 75° C. for 24 h. The reaction mixture was quenched with aqueous HCl 1 M until the pH of the solution was adjusted to 6.5/7. MeOH was evaporated, and the solid residue was triturated with water (100 mL). The solid was filtered, washed with water (20 mL) and dried under vacuum to give the title compound (1.6 g, yield: 86%) as a white solid. LC-MS m/z [M+H]⁺: 285.8; purity: 100%. ¹H NMR (400 MHz, DMSO-d₆) δ 10.41 (s, 1H), 8.53 (d, J=2.1 Hz, 1H), 8.07 (d, J=1.9 Hz, 1H), 7.41-7.32 (m, 1H), 6.22 (s, 1H), 3.92 (d, J=1.6 Hz, 6H), 3.52 (q, J=6.5 Hz, 1H), 1.45 (d, J=1.6 Hz, 3H).

### Intermediate NN203: 1,9-dichloro-3-cyclopropyl-5,7-dihydropyrido[4,3-d][3]benzazepin-6-one

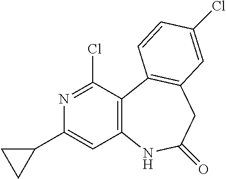 [SMILES: O=C1Cc2cc(Cl)ccc2-c2c(cc(C3CC3)nc2Cl)N1]

Under argon, a solution of 1,9-dichloro-5,7-dihydropyrido[4,3-d][3]benzazepin-6-one NN204 (10.8 mg, 0.0387 mmol), cyclopropanecarbonyl cyclopropanecarboperoxoate (21 mg, 0.1172 mmol), [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate (2 mg, 0.0018 mmol) in dry acetonitrile (0.2 mL) and trifluoroacetic acid (0.2 mL, 3 mmol) (previously sparge with argon for 5 min) was irradiated in the Pennoc for 3h30 at 450 nm (100% LED) (fan/stirring speed 400/4000). Then the reaction was concentrated under vacuo and purified by reverse phase chromatography (2 successive injections in basic mode) to give the title product (0.580 mg, 0.0018 mmol, yield: 4.7%) as a white solid. LC-MS m/z: [M+H]⁺: 319/321; purity: 98.75%. ¹H NMR (500 MHz, DMSO-d₆) δ 10.49 (s, 1H), 7.75 (d, J=8.5 Hz, 1H), 7.57 (d, J=2.3 Hz, 1H), 7.48-7.42 (m, 1H), 7.06 (s, 1H), 3.55-3.45 (m, 2H), 2.14-2.06 (m, 1H), 1.05-1.00 (m, 2H), 0.94 (m, 2H).

### Intermediate NN205: 1-chloro-3-(difluoromethyl)-5,7-dihydropyrido[4,3-d][3]benzazepin-6-one

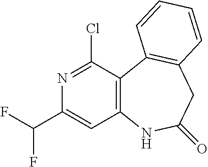 [SMILES: O=C1Cc2ccccc2-c2c(cc(C(F)F)nc2Cl)N1]

Under argon a solution 1-chloro-5,7-dihydropyrido[4,3-d][3]benzazepin-6-one NN206 (12 mg, 0.04904 mmol, difluoromethane sulfinic acid sodium salt (22 mg, 0.1514 mmol) and [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate (1 mg, 0.0009 mmol) in dry acetonitrile (0.25 mL) and trifluoroacetic acid (0.25 mL, 3.4 mmol) was irradiated in the Pennoc for 2 h at 450 nm (100% LED) (fan/stirring speed 400/4000). Then the reaction was concentrated under vacuo and purified by reverse phase chromatography (2 successive injections in basic mode) to give the title product (0.480 mg, 0.0016 mmol, yield: 3.3%) as a white solid. LC-MS m/z: [M+H]⁺: 295; purity: 99.9%. ¹H NMR (500 MHz, DMSO-d₆) δ 10.74 (s, 1H), 7.81 (d, J=7.8 Hz, 1H), 7.54-7.40 (m, 4H), 7.04 (t, J=54.6 Hz, 1H), 3.60 (d, J=12.9 Hz, 1H), 3.53-3.46 (d, J=12.9 Hz, 1H).

### Intermediate NN207: 3-chloro-5-(difluoromethyl)-10-methyl-4,8,12-triazatricyclo[9.4.0.0^{2,7}]pentadeca-1(11),2(7),3,5,12,14-hexaen-9-one

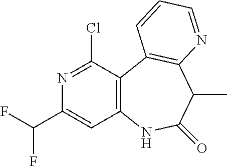 [SMILES: CC1C(=O)Nc2cc(C(F)F)nc(Cl)c2-c2cccnc21]

Under argon a solution 3-chloro-10-methyl-4,8,12-triazatricyclo[9.4.0.0²,7]pentadeca-1(11),2(7),3,5,12,14-hexaen-9-one NN168 (50 mg, 0.19 mmol) and bis(difluoroacetoxy)iodo)benzene (228 mg, 0.5786 mmol) in dry dichloromethane 1.0 mL and difluoroacetic acid (0.025 mL, 0.40 mmol) was irradiated in the Pennoc for 2h30 at 365 nm (100% LED) (fan/stirring speed 400/4000). Then the reaction was concentrated under vacuo and purified by reverse phase chromatography (1 acid purification followed by a second purification in basic mode) to give the title product (1.2 mg, 0.004 mmol, yield: 2%) as a white solid. LC-MS m/z: [M+H]⁺: 310/312; purity: 91.2%. ¹H NMR (400 MHz, DMSO-d₆) δ 10.97 (s, 1H), 8.70 (dd, J=4.7, 1.7 Hz, 1H), 8.27 (dd, J=8.0, 1.7 Hz, 1H), 7.49 (q, J=3.9 Hz, 2H), 7.06 (t, J=54.5 Hz, 1H), 3.74 (q, J=6.5 Hz, 1H), 1.49 (d, J=6.6 Hz, 3H).

The following intermediate esters have been prepared following similar procedures as the ones described for the preparation of related esters, such as Intermediates N68_5, N69_3, N70_6, N71_3 and N72_7 starting from the corresponding lactams intermediates (specific intermediates referenced in the table below).

| # | Structure | Chemical name | Method and Characterization |
|---|---|---|---|
| NN383 | 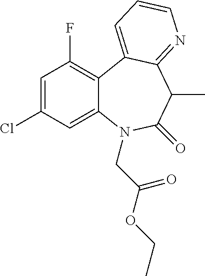 [SMILES: CCOC(=O)CN1C(=O)C(C)c2ncccc2-c2c(F)cc(Cl)cc21] | ethyl 2-(9-chloro- 11-fluoro-5- methyl-6-oxo-5H- pyrido[2,3- d][1]benzazepin-7- yl)acetate | Starting from 9-chloro-11-fluoro-5-methyl- 5,7-dihydropyrido[2,3-d][1]benzazepin-6- one NN217 at RT. LC-MS m/z [M + H]⁺: 363.2, 365.2; purity: 94%. ¹H NMR (400 MHz, CDCl₃) δ 8.69 (dd, J = 4.8, 1.7 Hz, 1H), 7.97 (ddd, J = 7.9, 4.5, 1.7 Hz, 1H), 7.34 (dd, J = 7.9, 4.8 Hz, 1H), 7.25 (t, J = 1.8 Hz, 1H), 7.17 (dd, J = 9.8, 2.0 Hz, 1H), 4.46 (d, J = 17.3 Hz, 1H), 4.30 (d, J = 17.3 Hz, 1H), 4.24-4.08 (m, 2H), 3.73-3.63 (m, 1H), 1.69 (d, J = 6.7 Hz, 3H), 1.20 (t, J = 7.1 Hz, 3H). |
| NN385 | 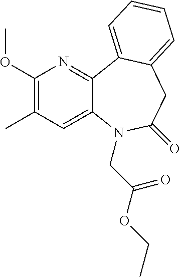 [SMILES: CCOC(=O)CN1C(=O)Cc2ccccc2-c2nc(OC)c(C)cc21] | ethyl 2-(2- methoxy-3-methyl- 6-oxo-7H- pyrido[3,2- d][3]benzazepin-5- yl)acetate | Starting from 2-methoxy-3-methyl-5,7- dihydropyrido[2,3-a][3]benzazepin-6-one NN337 at RT. LC-MS m/z [M − 14 + H]⁺: 341.2; purity: 89%. |
| NN386 | 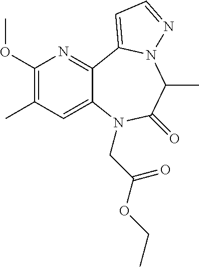 [SMILES: CCOC(=O)CN1C(=O)C(C)n2nccc2-c2nc(OC)c(C)cc21] | ethyl 2-(13- methoxy-7,12- dimethyl-8-oxo- 5,6,9,14- tetrazatricyclo[8.4. 0.0^{2,6}]tetradeca- 1(14),2,4,10,12- pentaen-9- yl)acetate | Starting from 13-methoxy-7,12-dimethyl- 5,6,9,14- tetrazatricyclo[8.4.0.0^{2,6}]tetradeca- 1(14),2,4,10,12-pentaen-8-one #353_4 at RT. LC-MS m/z [M + H]⁺: 345.2; purity: 96%. ¹H NMR (400 MHz, DMSO-d₆) δ 7.73 (s, 1H), 7.59 (d, J = 1.9 Hz, 1H), 6.77 (s, 1H), 4.82 (d, J = 6.4 Hz, 1H), 4.52 (d, J = 17.4 Hz, 1H), 4.43 (d, J = 17.4 Hz, 1H), 4.06-3.97 (m, 2H), 3.95 (s, 3H), 2.22 (s, 3H), 1.69 (d, J = 6.8 Hz, 3H), 1.07 (t, J = 7.1 Hz, 3H). |
| NN387 | 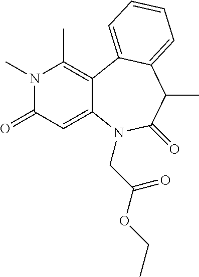 [SMILES: CCOC(=O)CN1C(=O)C(C)c2ccccc2-c2c1cc(=O)n(C)c2C] | ethyl 2-(1,2,7- trimethyl-3,6- dioxo-7H- pyrido[3,4- a][3]benzazepin-5- yl)acetate | Starting from 3-methoxy-1,7-dimethyl-5,7- dihydropyrido[4,3-d][3]benzazepin-6-one NN338 at RT. ¹H NMR (400 MHz, DMSO- d₆) δ 7.55 (dd, J = 7.6, 1.4 Hz, 1H), 7.44 (td, J = 7.5, 1.4 Hz, 1H), 7.40-7.31 (m, 2H), 6.65 (s, 1H), 4.54-4.34 (m, 2H), 4.04- 3.97 (m, 2H), 3.91 (s, 3H), 3.53 (q, J = 6.7 Hz, 1H), 2.48 (s, 3H), 1.43 (d, J = 6.8 Hz, 3H), 1.05 (t, J = 7.1 Hz, 3H). |
| NN388 | 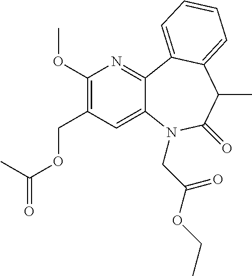 | ethyl 2-[3- (acetoxymethyl)-2- methoxy-7-methyl- 6-oxo-7H- pyrido[2,3- a][3]benzazepin-5- yl]acetate | Starting from 3-(hydroxymethyl)-2- methoxy-7-methyl-5,7-dihydropyrido[2,3- a][3]benzazepin-6-one NN368 in the presence of 0.1 eq. KI at 60° C. LC-MS m/z [M + H]⁺: 371.2; purity: 60%. |
| NN389 | 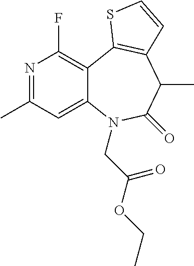 | ethyl 2-(14-fluoro- 7,12-dimethyl-8- oxo-3-thia-9,13- diazatricyclo[8.4.0. 0^{2,6}]tetradeca- 1(14),2(6),4,10,12- pentaen-9- yl)acetate | Starting from 14-fluoro-7,12-dimethyl-3- thia-9,13- diazatricyclo[8.4.0.0^{2,6}]tetradeca- 1(14),2(6),4,10,12-pentaen-8-one NN369 in the presence of 0.1 eq. KI at RT. LC-MS m/z [M + H]⁺: 349.2; purity: 89%. ¹H NMR (500 MHz, DMSO-d₆) δ 7.85 (d, J = 5.2 Hz, 1H), 7.30 (s, 1H), 7.12 (d, J = 5.3 Hz, 1H), 4.52 (s, 2H), 4.06 (qd, J = 7.2, 1.3 Hz, 2H), 3.28 (d, J = 6.9 Hz, 1H), 2.47 (s, 3H), 1.51 (d, J = 6.8 Hz, 3H), 1.10 (t, J = 7.1 Hz, 3H). |
| NN390 | 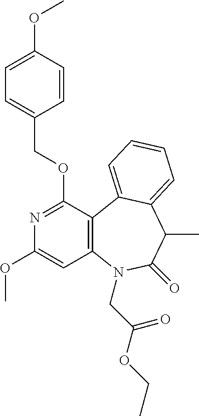 [SMILES: CCOC(=O)CN1C(=O)C(C)c2ccccc2-c2c1cc(OC)nc2OCc1ccc(OC)cc1] | ethyl 2-[3- methoxy-1-[(4- methoxyphenyl) methoxy]-7-methyl- 6-oxo-7H- pyrido[3,4- a][3]benzazepin-5- yl]acetate | Starting from 3-methoxy-1-[(4- methoxyphenyl)methoxy]-7-methyl-5,7- dihydropyrido[3,4-a][3]benzazepin-6-one NN370 at RT. LC-MS m/z [M + H]⁺: 477.2; purity: 95%. |
| NN391 | 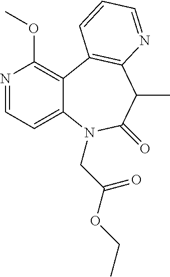 [SMILES: CCOC(=O)CN1C(=O)C(C)c2ncccc2-c2c1ccnc2OC] | ethyl 2-(3- methoxy-10- methyl-9-oxo- 4,8,12- triazatricyclo[9.4.0. 0^{2,7}]pentadeca- 1(11),2,4,6,12,14- hexaen-8- yl)acetate | Starting from 3-methoxy-10-methyl- 4,8, 12-triazatricyclo[9.4.0.0^{2,7}]pentadeca- 1(11),2,4,6,12,14-hexaen-9-one NN371 at RT. LC-MS m/z [M + H]⁺: 342.2; purity: 97%. ¹H NMR (400 MHz, DMSO-d₆) δ 8.60 (dd, J = 4.8, 1.7 Hz, 1H), 8.23 (d, J = 5.7 Hz, 1H), 8.16 (dd, J = 8.0, 1.7 Hz, 1H), 7.42 (dd, J = 7.9, 4.7 Hz, 1H), 7.11 (d, J = 5.8 Hz, 1H), 4.58-4.40 (m, 2H), 4.00 (q, J = 7.1 Hz, 2H), 3.91 (s, 3H), 3.64 (q, J = 6.6 Hz, 1H), 1.47 (d, J = 6.6 Hz, 3H), 1.04 (t, J = 7.1 Hz, 3H). |
| NN392 | 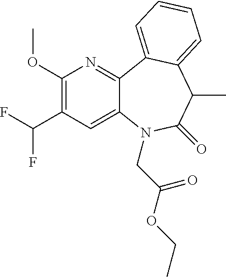 [SMILES: CCOC(=O)CN1C(=O)C(C)c2ccccc2-c2nc(OC)c(C(F)F)cc21] | ethyl 2-[3- (difluoromethyl)-2- methoxy-7-methyl- 6-oxo-7H- pyrido[3,2- d][3]benzazepin-5- yl]acetate | Starting from 3-(difluoromethyl)-2- methoxy-7-methyl-5,7-dihydropyrido[3,2- d][3]benzazepin-6-one NN372 at RT. LC- MS m/z [M + H]⁺: 391.0; purity: 100%. ¹H NMR (400 MHz, DMSO-d₆) δ 8.04-7.98 (m, 2H), 7.59 (td, J = 7.6, 1.5 Hz, 1H), 7.51 (td, J = 7.7, 1.2 Hz, 1H), 7.39 (d, J = 7.8 Hz, 1H), 7.14 (t, J = 54.4 Hz, 1H), 4.55- 4.34 (m, 2H), 4.04 (s, 3H), 4.01-3.92 (m, 2H), 3.53 (q, J = 6.7 Hz, 1H), 1.48 (d, J = 6.8 Hz, 3H), 1.00 (t, J = 7.1 Hz, 3H). |
| NN393 | 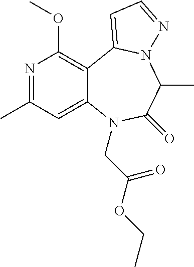 | ethyl 2-(14- methoxy-7,12- dimethyl-8-oxo- 5,6,9,13- tetrazatricyclo[8.4. 0.0^{2,6}]tetradeca- 1(14),2,4,10,12- pentaen-9- yl)acetate | Starting from 14-methoxy-7,12-dimethyl- 5,6,9,13- tetrazatricyclo[8.4.0.0^{2,6}]tetradeca- 1(14),2,4,10,12-pentaen-8-one NN379 at RT. LC-MS m/z [M + H]⁺: 345.2; purity: 96%. ¹H NMR (400 MHz, DMSO-d₆) δ 7.57 (d, J = 1.9 Hz, 1H), 6.98 (s, 1H), 6.66 (d, J = 2.0 Hz, 1H), 4.83 (q, J = 6.6 Hz, 1H), 4.51 (s, 1H), 4.50 (s, 1H), 4.04 (qd, J = 7.1, 1.7 Hz, 2H), 3.93 (s, 3H), 2.46 (s, 3H), 1.68 (d, J = 6.7 Hz, 3H), 1.10 (t, J = 7.1 Hz, 3H); purity: 95%. |
| NN394 | 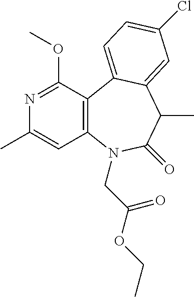 | ethyl 2-(9-chloro- 1-methoxy-3,7- dimethyl-6-oxo- 7H-pyrido[3,4- a][3]benzazepin-5- yl)acetate | Starting from 9-chloro-1-methoxy-3,7- dimethyl-5,7-dihydropyrido[3,4- a][3]benzazepin-6-one NN373 in the presence of 0.1 eq. KI at RT. LC-MS m/z [M + H]⁺: 389.2, 391.2; purity: 95%. ¹H NMR (400 MHz, CDCl₃) δ 7.68 (d, J = 8.3 Hz, 1H), 7.35-7.30 (m, 2H), 6.69 (d, J = 0.8 Hz, 1H), 4.47 (d, J = 17.3 Hz, 1H), 4.24-4.12 (m, 3H), 4.04 (s, 3H), 3.48 (q, J = 6.9 Hz, 1H), 2.57-2.52 (m, 3H), 1.58 (d, J = 6.8 Hz, 3H), 1.20 (t, J = 7.1 Hz, 3H). |
| NN395 | 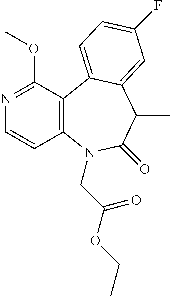 [SMILES: CCOC(=O)CN1C(=O)C(C)c2cc(F)ccc2-c2c1ccnc2OC] | ethyl 2-(9-fluoro-1- methoxy-7-methyl- 6-oxo-7H- pyrido[4,3- d][3]benzazepin-5- yl)acetate | Starting from 9-fluoro-1-methoxy-7- methyl-5,7-dihydropyrido[4,3- d][3]benzazepin-6-one NN374 at RT. LC- MS m/z [M + H]⁺: 359.2; purity: 96%. ¹H NMR (400 MHz, DMSO-d₆) δ 8.20 (d, J = 5.7 Hz, 1H), 7.78 (dd, J = 8.7, 5.9 Hz, 1H), 7.22 (td, J = 8.7, 2.7 Hz, 1H), 7.16 (dd, J = 10.1, 2.7 Hz, 1H), 7.06 (d, J = 5.8 Hz, 1H), 4.59-4.32 (m, 2H), 4.05-3.95 (m, 2H), 3.90 (s, 3H), 3.47 (q, J = 6.7 Hz, 1H), 1.43 (d, J = 6.7 Hz, 3H), 1.05 (t, J = 7.1 Hz, 3H). |
| NN396 | 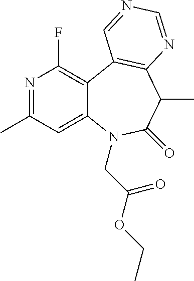 | ethyl 2-(15-fluoro- 8,13-dimethyl-9- oxo-4,6,10,14- tetrazatricyclo[9.4. 0.0^{2,7}]pentadeca- 1(15),2(7),3,5,11,1 3-hexaen-10- yl)acetate | Starting from 15-fluoro-8, 13-dimethyl- 4,6,10,14- tetrazatricyclo[9.4.0.0^{2,7}]pentadeca- 1(11),2,4,6,12,14-hexaen-9-one N36 at RT. LC-MS m/z [M + H]⁺: 345.2; purity: 82%. ¹H NMR (400 MHz, DMSO-d₆) δ 9.25 (s, 1H), 9.12 (d, J = 4.6 Hz, 1H), 7.41 (s, 1H), 4.60 (d, J = 17.5 Hz, 1H), 4.54 (d, J = 17.5 Hz, 1H), 4.05-3.95 (m, 2H), 3.92 (q, J = 6.5 Hz, 1H), 2.52 (s, 3H), 1.47 (d, J = 6.6 Hz, 3H), 1.03 (t, J = 7.1 Hz, 3H). |
| NN397 | 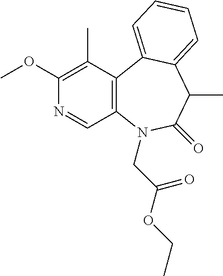 | ethyl 2-(2- methoxy-1,7- dimethyl-6-oxo- 7H-pyrido[4,3- a][3]benzazepin-5- yl)acetate | Starting from 2-methoxy-1,7-dimethyl-5,7- dihydropyrido[4,3-a][3]benzazepin-6-one NN375 at RT. LC-MS m/z [M + H]⁺: 355.2; purity: 95%. |
| NN398 | 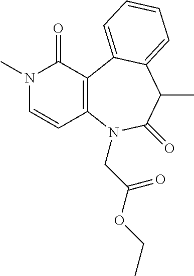 | ethyl 2-(2,7- dimethyl-1,6- dioxo-7H- pyrido[4,3- d][3]benzazepin-5- yl)acetate | Starting from 2,7-dimethyl-5,7- dihydropyrido[4,3-d][3]benzazepine-1,6- dione NN376 at RT. LC-MS m/z [M + H]⁺: 341.2; purity: 100%. ¹H NMR (400 MHz, DMSO-d₆) δ 7.83 (dd, J = 7.9, 1.4 Hz, 1H), 7.79 (d, J = 7.5 Hz, 1H), 7.41 (td, J = 7.5, 1.4 Hz, 1H), 7.31 (ddd, J = 8.7, 7.5, 3.9 Hz, 2H), 6.33 (d, J = 7.5 Hz, 1H), 4.45-4.26 (m, 2H), 4.01 (q, J = 7.1 Hz, 2H), 3.50 (s, 3H), 3.41 (q, J = 6.7 Hz, 1H), 1.46 (d, J = 6.8 Hz, 3H), 1.05 (t, J = 7.1 Hz, 3H). |
| NN399 | 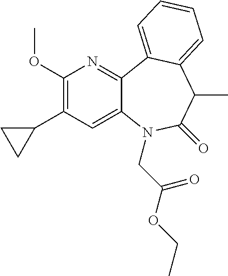 [SMILES: CCOC(=O)CN1C(=O)C(C)c2ccccc2-c2nc(OC)c(C3CC3)cc21] | ethyl 2-(3- cyclopropyl-2- methoxy-7-methyl- 6-oxo-7H- pyrido[2,3- a][3]benzazepin-5- yl)acetate | Starting from 3-cyclopropyl-2-methoxy-7- methyl-5,7-dihydropyrido[2,3- a][3]benzazepin-6-one NN377 in the presence of 0.1 eq. KI at RT. LC-MS m/z [M + H]⁺: 381.2; purity: 98%. ¹H NMR (400 MHz, DMSO-d₆) δ 7.95 (dd, J = 7.7, 1.6 Hz, 1H), 7.52 (td, J = 7.5, 1.6 Hz, 1H), 7.46 (t, J = 7.6 Hz, 1H), 7.33 (d, J = 7.7 Hz, 1H), 7.29 (s, 1H), 4.48 (d, J = 17.3 Hz, 1H), 4.34 (d, J = 17.3 Hz, 1H), 4.00 (s, 3H), 3.97- 3.87 (m, 2H), 3.42 (q, J = 6.7 Hz, 1H), 2.11- 2.02 (m, 1H), 1.46 (d, J = 6.8 Hz, 3H), 1.01-0.92 (m, 5H), 0.88-0.73 (m, 2H). |
| NN400 | 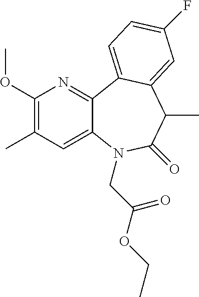 [SMILES: CCOC(=O)CN1C(=O)C(C)c2cc(F)ccc2-c2nc(OC)c(C)cc21] | ethyl 2-(9-fluoro-2- methoxy-3,7- dimethyl-6-oxo- 7H-pyrido[2,3- a][3]benzazepin-5- yl)acetate | Starting from 9-fluoro-2-methoxy-3,7- dimethyl-5,7-dihydropyrido[2,3- a][3]benzazepin-6-one NN378 at RT. LC- MS m/z [M + H]⁺: 373.2; purity: 100%. ¹H NMR (400 MHz, DMSO-d₆) δ 7.99 (dd, J = 8.7, 6.1 Hz, 1H), 7.67 (d, J = 1.1 Hz, 1H), 7.31 (td, J = 8.6, 2.6 Hz, 1H), 7.15 (dd, J = 10.3, 2.6 Hz, 1H), 4.53-4.26 (m, 2H), 4.16 (q, J = 7.1 Hz, 2H), 3.97 (s, 3H), 3.47 (q, J = 6.8 Hz, 1H), 2.22 (s, 3H), 1.45 (d, J = 6.8 Hz, 3H), 1.00 (t, J = 7.1 Hz, 3H). |
| NN153 | 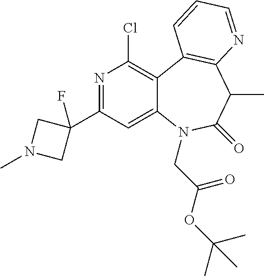 | tert-butyl 2-[3- chloro-5-(3-fluoro- 1-methyl-azetidin- 3-yl)-10-methyl-9- oxo-4,8,12- triazatricyclo[9.4.0. 0^{2,7}]pentadeca- 1(11),2,4,6,12,14- hexaen-8- yl]acetate | Starting form 3-chloro-5-(3-fluoro-1- methyl-azetidin-3-yl)-10-methyl-4,8,12- triazatricyclo[9.4.0.0^{2,7}]pentadeca- 1(11),2,4,6,12,14-hexaen-9-one NN152, tert-butyl bromoacetate, silver nitrate and using NaH as a base at RT, LC-MS m/z [M + H]⁺: 461.1/462.9; purity: 98%. TOF MS-MS m/z [M − C₅H₁₂N]⁺: 362.07/364.07 for fragment C₁₈H₁₄N₃O₃ClF |
| NN154 | 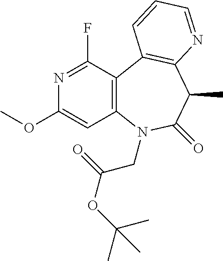 | Enantiomer (10S) or (10R) of tert- butyl 2-(3-fluoro-5- methoxy-10- methyl-9-oxo- 4,8,12- triazatricyclo[9.4.0. 0^{2,7}]pentadeca- 1(11),2(7),3,5,12,14- hexaen-8- yl)acetate | Starting from enantiomer (10S) or (10R) of 3-fluoro-5-methoxy-10-methyl-4,8,12- triazatricyclo[9.4.0.0^{2,7}]pentadeca- 1(11),2(7),3,5,12,14-hexaen-9-one (Intermediate N58_A), tert-butyl bromoacetate, K₂CO₃, KI in DMF at RT. LC-MS m/z: [M + H]⁺: 388.2; purity: 97%. ¹H NMR (400 MHz, DMSO-d₆) δ 8.63 (dd, J = 4.8, 1.7 Hz, 1H), 8.08 (ddd, J = 7.9, 4.5, 1.7 Hz, 1H), 7.47 (dd, J = 7.9, 4.8 Hz, 1H), 6.80 (s, 1H), 4.52-4.39 (m, 2H), 3.93 (s, 3H), 3.81 (q, J = 6.6 Hz, 1H), 1.48 (d, J = 6.6 Hz, 3H), 1.25 (s, 9H). ¹⁹F NMR (376 MHz, DMSO-d₆) δ −71.73 (d, J = 4.5 Hz). Chiral purity: 100%; rt = 4.22 min (second eluting enantiomer). For information, first eluting enantiomer rt = 3.22 min. Both measured by HPLC (Chiralpak IG-u from Daicel, EtOH 50% - heptane 50% - DEA 0.1%). |
| NN154a | 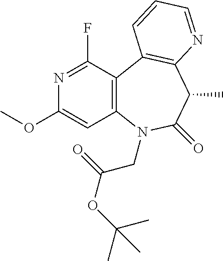 | Enantiomer (10S) or (10R) of tert- butyl 2-[3-chloro- 5,10-dimethyl-9- oxo-4,8,12- triazatricyclo[9.4.0. 0^{2,7}]pentadeca- 1(11),2(7),3,5,12, 14-hexaen-8- yl acetate | Starting from enantiomer (10S) or (10R) of 3-chloro-5,10-dimethyl-4,8,12- triazatricyclo[9.4.0.0^{2,7}]pentadeca- 1(11),2(7),3,5,12,14-hexaen-9-one (Intermediate N31_A), tert-butyl bromoacetate, KI at RT. LC-MS m/z: [M + H]⁺: 388.1; purity: 98.9%. |

The following intermediates NN191, NN197, NN200, NN230, NN209a have been prepared following the General Procedure 1 starting from 2-chloro-N-[4-(difluoromethyl)phenyl]acetamide (CAS 872533-93-2) and the corresponding lactams intermediates (specific intermediates referenced in the table below), unless stated or shown otherwise.

| # | Structure | Chemical name | Method and Characterization |
|---|---|---|---|
| NN191 | 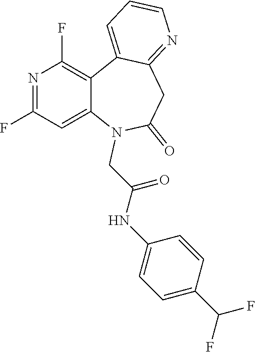 [SMILES: O=C(CN1C(=O)Cc2ncccc2-c2c1cc(F)nc2F)Nc1ccc(C(F)F)cc1] | N-[4- (difluoromethyl) phenyl]-2-(3,5- difluoro-9-oxo- 4,8,12- triazatricyclo[9.4.0. 0^{2,7}]pentadeca- 1(11),2(7),3,5,12,1 4-hexaen-8- yl)acetamide | Starting from 3,5-difluoro-4,8,12- triazatricyclo[9.4.0.0^{2,7}]pentadeca- 1(11),2(7),3,5,12,14-hexaen-9-one N117 and KI. LC-MS m/z [M + H]⁺: 430.9; purity: 99% |
| NN197 | 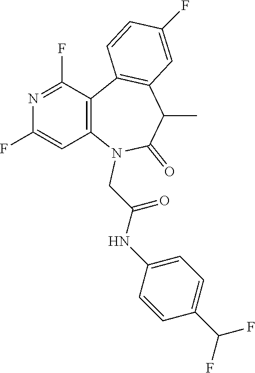 [SMILES: CC1C(=O)N(CC(=O)Nc2ccc(C(F)F)cc2)c2cc(F)nc(F)c2-c2ccc(F)cc21] | N-[4- (difluoromethyl) phenyl]-2-(1,3,9- trifluoro-7-methyl- 6-oxo-7H- pyrido[4,3- d][3]benzazepin-5- yl)acetamide | Starting from 1,3,9-trifluoro-7-methyl-5,7- dihydropyrido[4,3-d][3]benzazepin-6-one NN196. LC-MS m/z: [M − H] ⁻: 460; purity: 98.5%. ¹H NMR (400 MHz, DMSO-d₆) δ 10.50 (s, 1H), 7.78 (dd, J = 8.8, 5.3 Hz, 1H), 7.69 (d, J = 8.2 Hz, 2H), 7.52 (d, J = 8.2 Hz, 2H), 7.36-7.25 (m, 3H), 6.97 (t, J = 56.3 Hz, 1H), 4.62 (s, 2H), 3.72 (d, J = 6.6 Hz, 1H), 1.46 (d, J = 6.7 Hz, 3H). |
| NN200 | 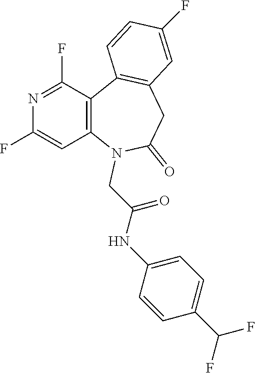 [SMILES: O=C(CN1C(=O)Cc2cc(F)ccc2-c2c1cc(F)nc2F)Nc1ccc(C(F)F)cc1] | N-[4- (difluoromethyl) phenyl]-2-(1,3,9- trifluoro-6-oxo-7H- pyrido[4,3- d][3]benzazepin-5- yl)acetamide | Starting form 1,3,9-trifluoro-5,7- dihydropyrido[4,3-d][3]benzazepin-6-one NN199. LC-MS m/z: [M − H]⁻: 446; purity: 98.5%. ¹H NMR (400 MHz, DMSO-d₆) δ 10.51 (s, 1H), 7.78 (dt, J = 9.9, 5.3 Hz, 1H), 7.70 (d, J = 8.2 Hz, 2H), 7.52 (d, J = 8.2 Hz, 2H), 7.46 (dd, J = 9.1, 2.8 Hz, 1H), 7.37-7.30 (m, 1H), 7.28 (s, 1H), 6.97 (t, J = 56.1 Hz, 1H), 4.61 (s, 2H), 3.77-3.62 (m, 2H). |
| NN230 | 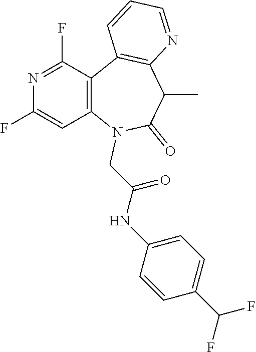 [SMILES: CC1C(=O)N(CC(=O)Nc2ccc(C(F)F)cc2)c2cc(F)nc(F)c2-c2cccnc21] | 2-(3,5-difluoro-10- methyl-9-oxo- 4,8,12- triazatricyclo[9.4.0. 0^{2,7}]pentadeca- 1(11),2(7),3,5,12,14- hexaen-8-yl)-N-[4- (difluoromethyl) phenyl]acetamide | Starting from 3,5-difluoro-10-methyl- 4,8, 12-triazatricyclo[9.4.0.0^{2,7}]pentadeca- 1(15),2(7),3,5,11,13-hexaen-9-one NN229. LC-MS m/z: [M + H]⁺: 445; purity: 100%. ¹H NMR (400 MHz, DMSO-d₆) δ 10.51 (s, 1H), 8.70 (dd, J = 4.8, 1.7 Hz, 1H), 8.17 (ddd, J = 8.0, 4.6, 1.7 Hz, 1H), 7.73-7.60 (m, 2H), 7.52 (dd, J = 8.0, 5.3 Hz, 3H), 7.34 (s, 1H), 7.13-6.80 (m, 1H), 4.11 (q, J = 5.2 Hz, 2H), 3.91 (q, J = 6.5 Hz, 1H), 1.50 (d, J = 6.7 Hz, 3H). |
| NN209a | 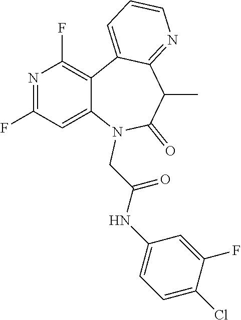 [SMILES: CC1C(=O)N(CC(=O)Nc2ccc(Cl)c(F)c2)c2cc(F)nc(F)c2-c2cccnc21] | N-(4-chloro-3- fluoro-phenyl)-2- (3,5-difluoro-10- methyl-9-oxo- 4,8,12- triazatricyclo[9.4.0. 0^{2,7}]pentadeca- 1(11),2(7),3,5,12, 14-hexaen-8- yl)acetamide | Starting from 3,5-difluoro-10-methyl- 4,8,12-triazatricyclo[9.4.0.0^{2,7}]pentadeca- 1(11),2(7),3,5,12,14-hexaen-9-one NN229, 2-chloro-N-(4-chloro-3-fluoro- phenyl)acetamide (CAS 895641-02-8). and KI. |
| NN209b | 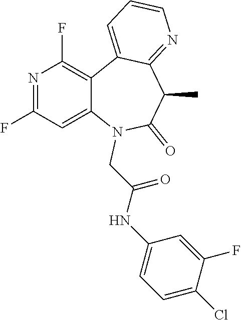 [SMILES: C[C@H]1C(=O)N(CC(=O)Nc2ccc(Cl)c(F)c2)c2cc(F)nc(F)c2-c2cccnc21] | N-(4-chloro-3- fluoro-phenyl)-2- [(10R)-3,5- difluoro-10-methyl- 9-oxo-4,8,12- triazatricyclo[9.4.0. 0^{2,7}]pentadeca- 1(11),2(7),3,5,12, 14-hexaen-8- yl]acetamide or N-(4-chloro-3- fluoro-phenyl)-2- [(10S)-3,5-difluoro- 10-methyl-9-oxo- 4,8,12- triazatricyclo[9.4.0. 0^{2,7}]pentadeca- 1(11),2(7),3,5,12, 14-hexaen-8- yl]acetamide | The above racemate NN209a was separated by chiral chromatography (SFC, Chiralpak IA from Daicel, CO₂ + ethanol 20%), chiral purity 100%, rt = 2.09 min. (second eluting enantiomer NN209b). For information, first eluting enantiomer NN209c rt = 1.60 min. Both measured by HPLC (HPLC-Chiralpak IA from Daicel, eluent EtOH:n-heptane 1:1 + 0.1% DEA, at 1.5 mL/min and 30° C.). LC-MS m/z [M + H]⁺: 447.09; purity: 99%. ¹H NMR (400 MHz, DMSO-d6) o 10.63 (s, 1H), 8.70 (dd, J = 4.8, 1.7 Hz, 1H), 8.17 (ddd, J = 8.0, 4.6, 1.7 Hz, 1H), 7.73 (dd, J = 11.9, 2.4 Hz, 1H), 7.52 (ddd, J = 8.7, 7.1, 2.3 Hz, 2H), 7.35-7.27 (m, 2H), 4.70-4.56 (m, 2H), 3.91 (q, J = 6.5 Hz, 1H), 1.49 (d, J = 6.6 Hz, 3H). |
| | or | | |
| | 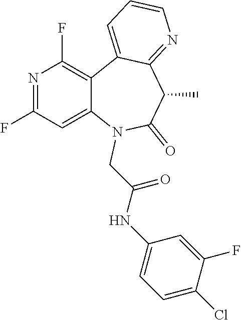 [SMILES: C[C@@H]1C(=O)N(CC(=O)Nc2ccc(Cl)c(F)c2)c2cc(F)nc(F)c2-c2cccnc21] | | |
| NN230a | 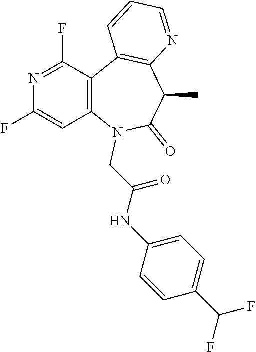 [SMILES: C[C@H]1C(=O)N(CC(=O)Nc2ccc(C(F)F)cc2)c2cc(F)nc(F)c2-c2cccnc21] | 2-[(10R)-3,5- difluoro-10-methyl- 9-oxo-4,8,12- triazatricyclo[9.4.0. 0^{2,7}]pentadeca- 1(11),2(7),3,5,12, 14-hexaen-8-yl]-N- [4- (difluoromethyl) phenyl]acetamide or 2-[(10S)-3,5- difluoro-10-methyl- 9-oxo-4,8,12- triazatricyclo[9.4.0. 0^{2,7}]pentadeca- 1(11),2(7),3,5,12, 14-hexaen-8-yl]-N- [4- (difluoromethyl) phenyl]acetamide | The above racemate NN230 was separated by chiral chromatography (SFC, ReproSil-Chiral-NR-R, CO₂ + isopropanol 20%), chiral purity 99.4%, rt = 2.01 min. (first eluting enantiomer NN230a). For information, second eluting enantiomer NN230b rt = 2.30 min. Both measured by HPLC (ReproSil-Chiral-NR-R, eluent EtOH:n-heptane 1:1 + 0.1% DEA, at 1.5 mL/min and 30° C.). |
| | or | | |
| | 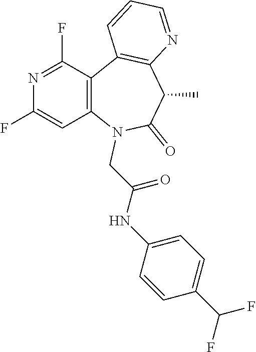 [SMILES: C[C@@H]1C(=O)N(CC(=O)Nc2ccc(C(F)F)cc2)c2cc(F)nc(F)c2-c2cccnc21] | | |

The following intermediates acids or lithium salt of acids have been prepared following similar procedures as the ones used for the preparation of related acids or lithium salt of acids, such as Intermediates N68-N72, N73_5, and N109-N111 by saponification of the corresponding ethyl esters intermediates using lithium hydroxide or by acidic hydrolysis of the corresponding tert-butyl ester intermediates using hydrochloric acid (specific intermediates referenced in the table below).

| # | Structure | Chemical name | Method and Characterization |
|---|---|---|---|
| NN403 | 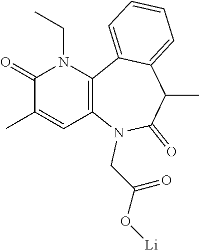 | [2-(1-ethyl-3,7- dimethyl-2,6- dioxo-7H- pyrido[3,2- d][3]benzazepin-5- yl)acetyl]oxylithium | Starting from ethyl 2-(1-ethyl-3,7-dimethyl- 2,6-dioxo-7H-pyrido[3,2-d][3]benzazepin- 5-yl)acetate #349_2. LC-MS m/z [M − Li + 2H]⁺: 341.2; purity: 98%. ¹H NMR (400 MHz, DMSO-d₆) δ 7.77 (s, 1H), 7.62-7.48 (m, 2H), 7.44-7.30 (m, 2H), 4.16 (dd, J = 13.9, 7.1 Hz, 1H), 3.89 (dd, J = 15.1, 9.7 Hz, 2H), 3.51 (q, J = 6.7 Hz, 1H), 3.22 (d, J = 16.3 Hz, 1H), 2.06 (s, 3H), 1.46 (d, J = 6.8 Hz, 3H), 0.96 (t, J = 6.9 Hz, 3H). |
| NN404 | 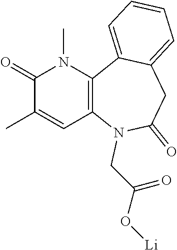 | [2-(1,3-dimethyl- 2,6-dioxo-7H- pyrido[3,2- d][3]benzazepin-5- yl)acetyl]oxylithium | Starting from ethyl 2-(1,3-dimethyl-2,6- dioxo-7H-pyrido[3,2-d][3]benzazepin-5- yl)acetate NN443. LC-MS m/z [M + 2H − Li]⁺: 313.2; purity: 79%. |
| NN405 | 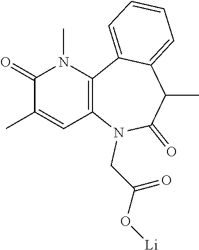 | [2-(7,12,14- trimethyl-8,13- dioxo-5,6,9,14- tetrazatricyclo[8.4. 0.0^{2,6}]tetradeca- 1(10),2,4,11- tetraen-9- yl)acetyl]oxylithium | Starting from ethyl 2-(7,12,14-trimethyl- 8,13-dioxo-5,6,9,14- tetrazatricyclo[8.4.0.0^{2,6}]tetradeca- 1(10),2,4, 11-tetraen-9-yl)acetate #353_6. LC-MS m/z [M − Li + 2H]⁺: 317.1; purity: 40%. ¹H NMR (400 MHz, DMSO-d₆) δ 7.51 (d, J = 1.8 Hz, 1H), 7.15 (s, 1H), 6.18 (d, J = 1.8 Hz, 1H), 4.21 (q, J = 7.2 Hz, 1H), 3.22 (d, J = 14.5 Hz, 1H), 3.10 (d, J = 14.5 Hz, 1H), 2.94 (s, 3H), 2.04 (d, J = 1.0 Hz, 3H), 1.56 (d, J = 7.2 Hz, 3H). |
| NN406 | 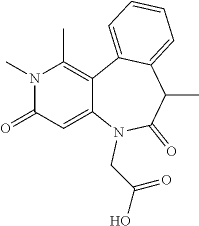 [SMILES: Cc1c2c(cc(=O)n1C)N(CC(=O)O)C(=O)C(C)c1ccccc1-2] | 2-(1,2,7-trimethyl- 3,6-dioxo-7H- pyrido[3,4- a][3]benzazepin-5- yl)acetic acid | Starting from ethyl 2-(1,2,7-trimethyl-3,6- dioxo-7H-pyrido[3,4-a][3]benzazepin-5- yl)acetate #355_4. LC-MS m/z [M + H]⁺: 327.1; purity: 85%. |
| NN407 | 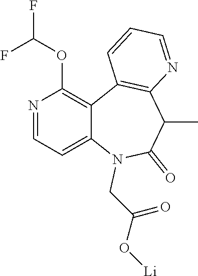 | [2-[3- (difluoromethoxy)- 10-methyl-9-oxo- 4,8,12- triazatricyclo[9.4.0. 0^{2,7}]pentadeca- 1(11),2,4,6,12,14- hexaen-8- yl]acetyl]oxylithium | Starting from ethyl 2-[3-(difluoromethoxy)- 10-methyl-9-oxo-4,8,12- triazatricyclo[9.4.0.0^{2,7}]pentadeca- 1(11),2,4,6,12,14-hexaen-8-yl]acetate #412_1. LC-MS m/z [M − Li + 2H]⁺: 350.2; purity: 78%. |
| NN155 | 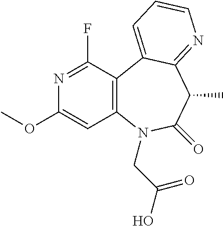 | Enantiomer (10R) or (10S) of 2-[3- fluoro-5-methoxy- 10-methyl-9-oxo- 4,8,12- triazatricyclo[9.4.0. 0^{2,7}]pentadeca- 1(11),2(7),3,5,12, 14-hexaen-8- yl]acetic acid | Starting from enantiomer (10S) or (10R) of tert-butyl 2-(3-fluoro-5-methoxy-10- methyl-9-oxo-4,8,12- triazatricyclo[9.4.0.0^{2,7}]pentadeca- 1(11),2(7),3,5, 12,14-hexaen-8-yl)acetate (Intermediate NN154). LC-MS m/z: [M + H]⁺: 332.1; purity: 94%. ¹H NMR (400 MHz, DMSO-d₆) δ 8.63 (dd, J = 4.8, 1.7 Hz, 1H), 8.08 (ddd, J = 7.9, 4.5, 1.7 Hz, 1H), 7.48 (dd, J = 7.9, 4.8 Hz, 1H), 6.82 (s, 1H), 4.45 (s, 2H), 3.92 (s, 3H), 3.84 (q, J = 6.6 Hz, 1H), 1.48 (d, J = 6.6 Hz, 3H). Proton for COOH not observed. ¹⁹F NMR (376 MHz, DMSO-d₆) δ -71.90 (d, J = 4.5 Hz). Chiral purity: 100%; rt = 2.74 min (first eluting enantiomer). For information, second eluting enantiomer rt = 3.72 min. Both measured by HPLC (Lux Cellulose-2 from Phenomenex, EtOH 50%-heptane 50%-DEA 0.1%). |
| | or | | |
| | 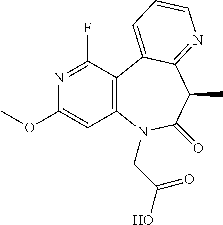 | | |
| NN155a | 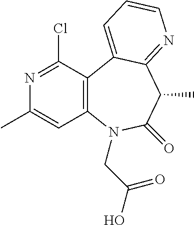 | Enantiomer (10R) or (10S) of 2-[3- chloro-5,10- dimethyl-9-oxo- 4,8,12- triazatricyclo[9.4.0. 0^{2,7}]pentadeca- 1(11),2(7),3,5,12, 14-hexaen-8- yl]acetic acid | Starting from enantiomer (10S) or (10R) of tert-butyl 2-[3-chloro-5,10-dimethyl-9-oxo- 4,8,12-triazatricyclo[9.4.0.0^{2,7}]pentadeca- 1(11),2(7),3,5,12,14-hexaen-8-yl]acetate NN154a. LC-MS m/z [M + H]⁺: 332; purity: 99.7%. |
| | or | | |
| | 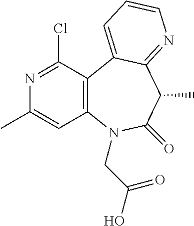 | | |
| NN408 | 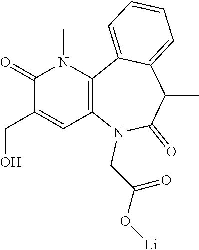 | [2-[3- (Hydroxymethyl)- 1,7-dimethyl-2,6- dioxo-7H- pyrido[3,2- d][3]benzazepin-5- yl]acetyl]oxylithium | Starting from ethyl 2-[3-(acetoxymethyl)- 1,7-dimethyl-2,6-dioxo-7H-pyrido[3,2- d][3]benzazepin-5-yl]acetate NN388. LC- MS m/z [M − Li + 2H]⁺: 343.2; purity: 100%. |
| NN409 | 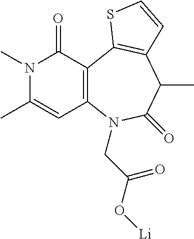 | [2-(7,12,13- Trimethyl-8,14- dioxo-3-thia-9,13- diazatricyclo[8.4.0. 0^{2,6}]tetradeca- 1(10),2(6),4,11- tetraen-9- yl)acetyl]oxylithium | Starting from ethyl 2-(7,12,13-trimethyl- 8,14-dioxo-3-thia-9,13- diazatricyclo[8.4.0.0^{2,6}]tetradeca- 1(10),2(6),4,11-tetraen-9-yl)acetate NN422. LC-MS m/z [M − Li + 2H]⁺: 333.2; purity: 99%. |
| NN410 | 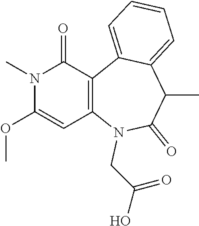 [SMILES: COc1cc2c(c(=O)n1C)-c1ccccc1C(C)C(=O)N2CC(=O)O] | 2-(3-Methoxy-2,7- dimethyl-1,6- dioxo-7H- pyrido[4,3- d][3]benzazepin-5- yl)acetic acid | Starting from ethyl 2-(3-methoxy-2,7- dimethyl-1,6-dioxo-7H-pyrido[4,3- d][3]benzazepin-5-yl)acetate NN424 LC- MS m/z [M + H]⁺: 343.2; purity: 95%. |
| NN411 | 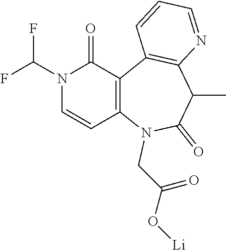 | [2-[4- (Difluoromethyl)- 10-methyl-3,9- dioxo-4,8,12- triazatricyclo[9.4.0. 0^{2,7}]pentadeca- 1(11),2(7),5,12,14- pentaen-8- yl]acetyl]oxylithium | Starting from ethyl 2-[4-(difluoromethyl)- 10-methyl-3,9-dioxo-4,8,12- triazatricyclo[9.4.0.0^{2,7}]pentadeca- 1(11),2(7),5,12,14-pentaen-8-yl]acetate #412_1. LC-MS m/z [M − Li + 2H]⁺: 350.2; purity: 100%. |
| NN412 | 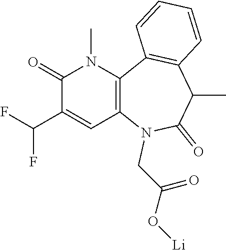 | [2-[3- (Difluoromethyl)- 1,7-dimethyl-2,6- dioxo-7H- pyrido[3,2- d][3]benzazepin-5- yl]acetyl]oxylithium | Starting from ethyl 2-[3-(difluoromethyl)- 1,7-dimethyl-2,6-dioxo-7H-pyrido[3,2- d][3]benzazepin-5-yl]acetate NN426. LC- MS m/z (M − Li + 2H)⁺: 363.0; purity: 100%. |
| NN413 | 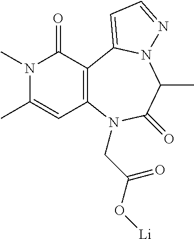 | [2-(7,12,13- trimethyl-8,14- dioxo-5,6,9,13- tetrazatricyclo[8.4. 0.0^{2,6}]tetradeca- 1(10),2,4,11- tetraen-9- yl)acetyl]oxylithium | Starting from ethyl 2-(7,12,13-trimethyl- 8,14-dioxo-5,6,9,13- tetrazatricyclo[8.4.0.0^{2,6}]tetradeca- 1(10),2,4, 11-tetraen-9-yl)acetate NN428. LC-MS m/z [M − Li + 2H]⁺: 317.2; purity: 52%. |
| NN414 | 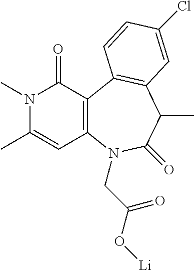 | [2-(9-Chloro-2,3,7- trimethyl-1,6- dioxo-7H- pyrido[4,3- d][3]benzazepin-5- yl)acetyl]oxylithium | Starting from ethyl 2-(9-chloro-2,3,7- trimethyl-1,6-dioxo-7H-pyrido[4,3- d][3]benzazepin-5-yl)acetate NN430. LC- MS m/z [M − Li + 2H]⁺: 361.1, 363.1; purity: 99%. |
| NN415 | 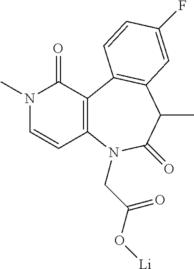 | [2-(9-Fluoro-2,7- dimethyl-1,6- dioxo-7H- pyrido[4,3- d][3]benzazepin-5- yl)acetyl]oxylithium | Starting from 2-(9-fluoro-2,7- dimethyl-1,6-dioxo-7H-pyrido[4,3- d][3]benzazepin-5-yl)acetate NN431. LC- MS m/z [M − Li + 2H]⁺: 331.2; purity: 95%. |
| NN416 | 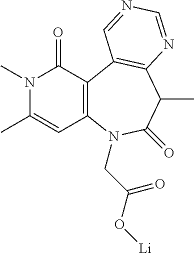 | [2-(8,13,14- Trimethyl-9,15- dioxo-4,6,10,14- tetrazatricyclo[9.4. 0.0^{2,7}]pentadeca- 1(11),2(7),3,5,12- pentaen-10- yl)acetyl]oxylithium | Starting from ethyl 2-(8,13,14-trimethyl- 9,15-dioxo-4,6,10,14- tetrazatricyclo[9.4.0.0^{2,7}]pentadeca- 1(11),2(7),3,5, 12-pentaen-10-yl)acetate NN433. LC-MS m/z [M − Li + 2H]⁺: 329.2; purity: 42%. |
| NN417 | 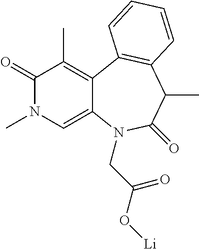 | [2-(1,3,7-trimethyl- 2,6-dioxo-7H- pyrido[3,4- d][3]benzazepin-5- yl)acetyl]oxylithium | Starting from ethyl 2-(1,3,7-trimethyl-2,6- dioxo-7H-pyrido[3,4-d][3]benzazepin-5- yl)acetate NN435. LC-MS m/z [M − Li + 2H]⁺: 327.2; purity: 97%. |
| NN418 | 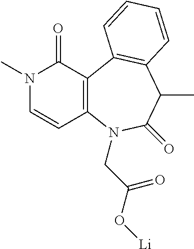 | [2-(2,7-Dimethyl- 1,6-dioxo-7H- pyrido[4,3- d][3]benzazepin-5- yl)acetyl]oxylithium | Starting from ethyl 2-(2,7-dimethyl-1,6- dioxo-7H-pyrido[4,3-d] [3]benzazepin-5- yl)acetate NN398. LC-MS m/z [M − Li + 2H]⁺: 313.2; purity: 100%. |
| NN419 | 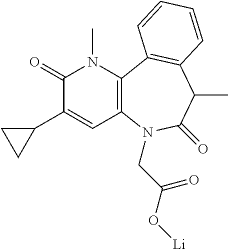 | [2-(3-Cyclopropyl- 1,7-dimethyl-2,6- dioxo-7H- pyrido[3,2- d][3]benzazepin-5- yl)acetyl]oxylithium | Starting from ethyl 2-(3-cyclopropyl-1,7- dimethyl-2,6-dioxo-7H-pyrido[3,2- d][3]benzazepin-5-yl)acetate NN436. LC- MS m/z [M − Li + 2H]⁺: 353.2; purity: 98%. ¹H NMR (400 MHz, DMSO-d₆) δ 7.62 (d, J = 7.7 Hz, 1H), 7.58 (s, 1H), 7.51 (t, J = 7.5 Hz, 1H), 7.40-7.31 (m, 2H), 3.89 (d, J = 16.1 Hz, 1H), 3.51 (d, J = 6.8 Hz, 1H), 3.15 (d, J = 16.0 Hz, 1H), 2.11-2.02 (m, 1H), 1.44 (d, J = 6.8 Hz, 3H), 0.92-0.86 (m, 2H), 0.65-0.57 (m, 2H). N—CH3 obscured by the water peak. |
| NN420 | 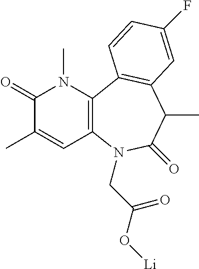 | [2-(9-Fluoro-1,3,7- trimethyl-2,6- dioxo-7H- pyrido[3,2- d][3]benzazepin-5- yl)acetyl]oxylithium | Starting from ethyl 2-(9-fluoro-1,3,7- trimethyl-2,6-dioxo-7H-pyrido[3,2- d][3]benzazepin-5-yl)acetate NN438. LC- MS m/z [M − Li + 2H]⁺: 345.2; purity: 100%. |

The following intermediate lactams have been prepared following similar procedures as the ones used for the preparation of related lactames, such as Intermediates N67_4, N69_4, N70_7, N71_4, N72_8, N73_5, #100_1, #184_2, #185_2, #355_3 or #397 starting from specific intermediates referenced in the table below. Intermediates NN421, and NN432 have been prepared by hydrolysis using HCl in dioxane at 10000 followed by H₂SO₄ in EtOH at 8000. Intermediate NN423 has been prepared using TEA in DCM at RT.

| # | Structure | Chemical name | Method and Characterization |
|---|---|---|---|
| NN421 | 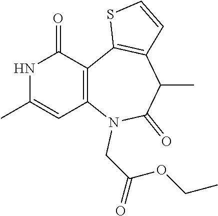 [SMILES: CCOC(=O)CN1C(=O)C(C)c2ccsc2-c2c1cc(C)[nH]c2=O] | Ethyl 2-(7,12- dimethyl-8,14- dioxo-3-thia-9,13- diazatricyclo[8.4.0. 0^{2,6}]tetradeca- 1(10),2(6),4,11- tetraen-9- yl)acetate | Starting from ethyl 2-(14-fluoro-7,12- dimethyl-8-oxo-3-thia-9,13- diazatricyclo[8.4.0.0^{2,6}]tetradeca- 1(14),2(6),4, 10,12-pentaen-9-yl)acetate NN389. LC-MS m/z [M + H]⁺: 347.2; purity: 75%. ¹H NMR (500 MHz, DMSO-d₆) δ 11.94 (s, 1H), 7.67 (d, J = 5.2 Hz, 1H), 6.97 (d, J = 5.2 Hz, 1H), 6.12 (s, 1H), 4.37 (s, 2H), 4.04 (t, J = 7.1 Hz, 2H), 3.05 (q, J = 6.8 Hz, 1H), 2.23 (s, 3H), 1.51 (d, J = 6.8 Hz, 3H), 1.10 (t, J = 7.1 Hz, 3H). |
| NN423 | 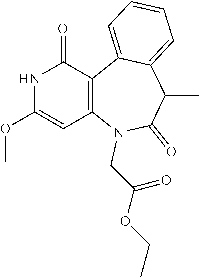 [SMILES: CCOC(=O)CN1C(=O)C(C)c2ccccc2-c2c1cc(OC)[nH]c2=O] | Ethyl 2-(3- methoxy-7-methyl- 1,6-dioxo-2,7- dihydropyrido[4,3- d][3]benzazepin-5- yl)acetate | Starting from ethyl 2-[3-methoxy-1-[(4- methoxyphenyl)methoxy]-7-methyl-6-oxo- 7H-pyrido[3,4-a][3]benzazepin-5- yl]acetate NN390. LC-MS m/z [M + H]⁺: 357.2; purity: 95%. ¹H NMR (400 MHz, DMSO-d₆) δ 11.58 (s, 1H), 7.74 (dd, J = 7.7, 1.5 Hz, 1H), 7.46-7.23 (m, 3H), 6.06 (s, 1H), 4.55-4.31 (m, 2H), 4.00 (q, J = 7.1 Hz, 2H), 3.97(3H, s), 3.45 (q, J = 6.7 Hz, 1H), 1.44 (d, J = 6.8 Hz, 3H), 1.04 (t, J = 7.1 Hz, 3H). |
| NN425 | 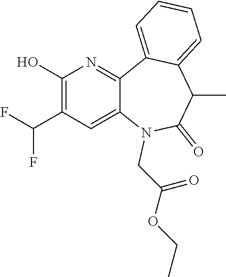 [SMILES: CCOC(=O)CN1C(=O)C(C)c2ccccc2-c2nc(O)c(C(F)F)cc21] | Ethyl 2-[3- (difluoromethyl)-2- hydroxy-7-methyl- 6-oxo-7H- pyrido[3,2- d][3]benzazepin-5- ylacetate | Starting from ethyl 2-[3-(difluoromethyl)-2- methoxy-7-methyl-6-oxo-7H-pyrido[3,2- d][3]benzazepin-5-yl]acetate NN392. LC- MS m/z [M + H]⁺: 377.2; purity: 94%. |
| NN427 | 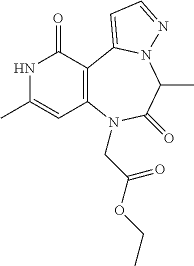 | Ethyl 2-(7,12- dimethyl-8,14- dioxo-5,6,9,13- tetrazatricyclo[8.4. 0.0^{2,6}]tetradeca- 1(10),2,4,11- tetraen-9- yl)acetate | Starting from ethyl 2-(14-methoxy-7,12- dimethyl-8-oxo-5,6,9,13- tetrazatricyclo[8.4.0.0^{2,6}]tetradeca- 1(14),2,4,10,12-pentaen-9-yl)acetate NN393. LC-MS m/z [M + H]⁺: 331.2; purity: 100%. |
| NN429 | 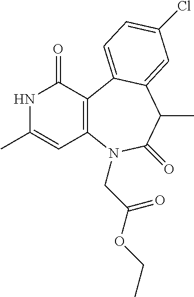 | Ethyl 2-(9-chloro- 3,7-dimethyl-1,6- dioxo-2,7- dihydropyrido[4,3- d][3]benzazepin-5- yl)acetate | Starting from ethyl 2-(9-chloro-1-methoxy- 3,7-dimethyl-6-oxo-7H-pyrido[3,4- a][3]benzazepin-5-yl)acetate NN394. LC- MS m/z [M + H]⁺: 375.1, 377.1; purity: 99%. ¹H NMR (400 MHz, DMSO-d₆) δ 11.91 (s, 1H), 7.85 (d, J = 8.5 Hz, 1H), 7.37 (dd, J = 8.5, 2.2 Hz, 1H), 7.27 (d, J = 2.2 Hz, 1H), 6.13 (s, 1H), 4.43-4.26 (m, 2H), 4.06- 4.00 (m, 2H), 3.43 (q, J = 6.7 Hz, 1H), 2.22 (s, 3H), 1.44 (d, J = 6.8 Hz, 3H), 1.04 (t, J = 7.1 Hz, 3H). |
| NN401 | 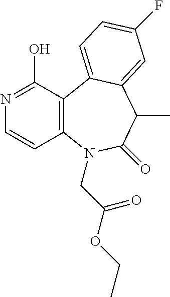 [SMILES: CCOC(=O)CN1C(=O)C(C)c2cc(F)ccc2-c2c1ccnc2O] | Ethyl 2-(9-fluoro-1- hydroxy-7-methyl- 6-oxo-7H- pyrido[4,3- d][3]benzazepin-5- yl)acetate | Starting from ethyl 2-(9-fluoro-1-methoxy- 7-methyl-6-oxo-7H-pyrido[4,3- d][3]benzazepin-5-yl)acetate NN395. LC- MS m/z [M + H]⁺: 345.2; purity: 97%. ¹H NMR (400 MHz, DMSO-d₆) δ 11.86 (s, 1H), 7.89 (dd, J = 8.8, 6.1 Hz, 1H), 7.46 (d, J = 7.2 Hz, 1H), 7.17 (td, J = 8.6, 2.7 Hz, 1H), 7.09 (dd, J = 10.2, 2.7 Hz, 1H), 6.26 (d, J = 7.3 Hz, 1H), 4.40 (d, J = 17.5 Hz, 1H), 4.32 (d, J = 17.5 Hz, 1H), 4.01 (q, J = 7.1 Hz, 2H), 3.44 (q, J = 6.7 Hz, 1H), 1.44 (d, J = 6.8 Hz, 3H), 1.05 (t, J = 7.1 Hz, 3H) purity: 83%. |
| NN432 | 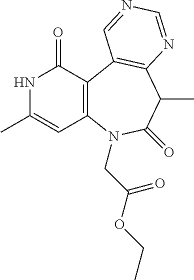 | Ethyl 2-(8,13- dimethyl-9,15- dioxo-4,6,10,14- tetrazatricyclo [9.4. 0.0^{2,7}]pentadeca- 1(11),2(7),3,5,12- pentaen-10- yl)acetate | Starting from ethyl 2-(15-fluoro-8,13- dimethyl-9-oxo-4,6,10,14- tetrazatricyclo[9.4.0.0^{2,7}]pentadeca- 1(15),2(7),3,5,11,13-hexaen-10-yl)acetate NN396. LC-MS m/z [M + H]⁺: 343.2; purity: 82%. |
| NN434 | 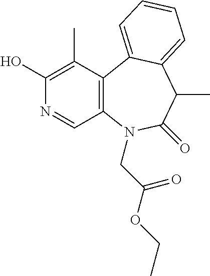 [SMILES: CCOC(=O)CN1C(=O)C(C)c2ccccc2-c2c1cnc(O)c2C] | Ethyl 2-(2- hydroxy-1,7- dimethyl-6-oxo- 7H-pyrido[3,4- d][3]benzazepin-5- yl)acetate | Starting from ethyl 2-(2-methoxy-1,7- dimethyl-6-oxo-7H-pyrido[3,4- d][3]benzazepin-5-yl)acetate NN397. LC- MS m/z [M + H]⁺: 341.2; purity: 95%. |
| NN436 | 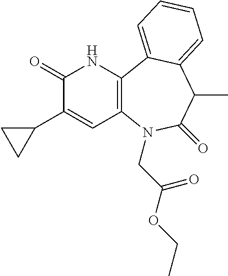 [SMILES: CCOC(=O)CN1C(=O)C(C)c2ccccc2-c2[nH]c(=O)c(C3CC3)cc21] | Ethyl 2-(3- cyclopropyl-7- methyl-2,6-dioxo- 1,7- dihydropyrido[3,2- d][3]benzazepin-5- yl)acetate | Starting from ethyl 2-(3-cyclopropyl-2- methoxy-7-methyl-6-oxo-7H-pyrido[3,2- d][3]benzazepin-5-yl)acetate NN399. LC- MS m/z [M + H]⁺: 367.2; purity: 97%. ¹H NMR (400 MHz, DMSO-d₆) δ 11.77 (s, 1H), 7.67 (d, J = 7.8 Hz, 1H), 7.54 (td, J = 7.6, 1.3 Hz, 1H), 7.43 (t, J = 7.3 Hz, 1H), 7.33 (d, J = 7.8 Hz, 1H), 7.08 (s, 1H), 4.41 (d, J = 17.2 Hz, 1H), 4.23 (d, J = 17.3 Hz, 1H), 3.89 (qd, J = 7.1, 5.7 Hz, 2H), 3.50 (q, J = 6.8 Hz, 1H), 2.09-2.01 (m, 1H), 1.45 (d, J = 6.8 Hz, 3H), 0.97-0.88 (m, 5H), 0.78 (tt, J = 13.1, 6.1 Hz, 2H). |
| NN437 | 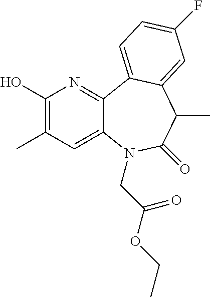 [SMILES: CCOC(=O)CN1C(=O)C(C)c2cc(F)ccc2-c2nc(O)c(C)cc21] | Ethyl 2-(9-fluoro-2- hydroxy-3,7- dimethyl-6-oxo- 7H-pyrido[2,3- a][3]benzazepin-5- yl)acetate | Starting from ethyl 2-(9-fluoro-2-methoxy- 3,7-dimethyl-6-oxo-7H-pyrido[2,3- a][3]benzazepin-5-yl)acetate NN400. LC- MS m/z [M + H]⁺: 359.2; purity: 99%. ¹H NMR (400 MHz, DMSO-d₆) δ 11.75 (brs, 1H), 7.73 (dd, J = 8.7, 5.8 Hz, 1H), 7.51 (s, 1H), 7.29 (td, J = 8.6, 2.6 Hz, 1H), 7.16 (dd, J = 10.2, 2.6 Hz, 1H), 4.45-4.18 (m, 2H), 3.93 (qd, J = 7.1, 4.1 Hz, 2H), 3.56 (q, J = 6.7 Hz, 1H), 2.08 (s, 3H), 1.44 (d, J = 6.8 Hz, 3H), 0.98 (t, J = 7.1 Hz, 3H). |
| NN441 | 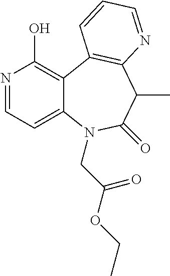 [SMILES: CCOC(=O)CN1C(=O)C(C)c2ncccc2-c2c1ccnc2O] | Ethyl 2-(3- hydroxy-10- methyl-9-oxo- 4,8,12- triazatricyclo[9.4.0. 0^{2,7}]pentadeca- 1(11),2,4,6,12,14- hexaen-8- yl)acetate | Starting from ethyl 2-(3-methoxy-10- methyl-9-oxo-4,8,12- triazatricyclo[9.4.0.0^{2,7}]pentadeca- 1(11),2,4,6,12,14-hexaen-8-yl)acetate NN391. LC-MS m/z [M + H]⁺: 328.2; purity: 97%. ¹H NMR (400 MHz, DMSO-d₆) δ 11.94 (s, 1H), 8.56 (dd, J = 4.7, 1.8 Hz, 1H), 8.25 (dd, J = 8.0, 1.7 Hz, 1H), 7.50 (d, J = 7.3 Hz, 1H), 7.37 (dd, J = 7.9, 4.7 Hz, 1H), 6.31 (d, J = 7.3 Hz, 1H), 4.49-4.30 (m, 2H), 4.01 (q, J = 7.1 Hz, 2H), 3.60 (q, J = 6.6 Hz, 1H), 1.49 (d, J = 6.6 Hz, 3H), 1.05 (t, J = 7.1 Hz, 3H). |
| NN442 | 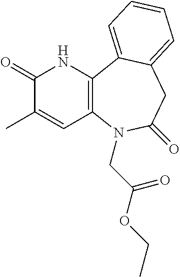 [SMILES: CCOC(=O)CN1C(=O)Cc2ccccc2-c2[nH]c(=O)c(C)cc21] | Ethyl 2-(3-methyl- 2,6-dioxo-1,7- dihydropyrido[3,2- d][3]benzazepin-5- yl)acetate | Starting from ethyl 2-(2-methoxy-3-methyl- 6-oxo-7H-pyrido[3,2-d][3]benzazepin-5- yl)acetate NN385. LC-MS m/z [M + H]⁺: 327.2; purity: 96%. ¹H NMR (400 MHz, DMSO-d₆) δ 11.70 (s, 1H), 7.70 (d, J = 7.4 Hz, 1H), 7.55-7.32 (m, 4H), 4.37 (d, J = 17.4 Hz, 1H), 4.23 (d, J = 17.4 Hz, 1H), 4.06-3.91 (m, 2H), 3.56 (d, J = 12.6 Hz, 1H), 3.52 (d, J = 12.5 Hz, 1H), 2.12-2.03 (m, 3H), 1.01 (t, J = 7.1 Hz, 3H). |

The following intermediate lactams have been prepared following similar procedures as the ones used for the preparation of related lactames, such as Intermediates #1843, #185 3, #353_6, #355 4 and #356_5 starting from specific intermediates referenced in the table below.

| # | Structure | Chemical name | Method and Characterization |
|---|---|---|---|
| NN422 | 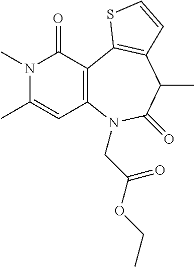 | Ethyl 2-(7,12,13- trimethyl-8,14- dioxo-3-thia-9,13- diazatricyclo[8.4.0. 0^{2,6}]tetradeca- 1(10),2(6),4,11- tetraen-9- yl)acetate | Starting from ethyl 2-(14-hydroxy-7,12- dimethyl-8-oxo-3-thia-9,13- diazatricyclo[8.4.0.0^{2,6}]tetradeca- 1(10),2(6),4,11,13-pentaen-9-yl)acetate NN421. LC-MS m/z [M + H]⁺: 361.2; rt: 0.93 min; purity: 99%. ¹H NMR (400 MHz, DMSO-d₆) δ 7.69 (d, J = 5.3 Hz, 1H), 6.98 (d, J = 5.3 Hz, 1H), 6.30 (s, 1H), 4.39 (s, 2H), 4.15-3.98 (m, 2H), 3.51 (s, 3H), 3.08- 3.01 (m, 1H), 2.42 (s, 3H), 1.51 (d, J = 6.8 Hz, 3H), 1.08 (d, J = 7.1 Hz, 3H); purity: 85%. |
| NN424 | 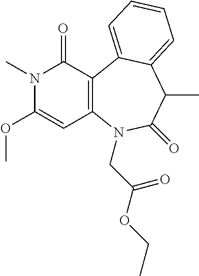 | Ethyl 2-(3- methoxy-2,7- dimethyl-1,6- dioxo-7H- pyrido[4,3- d][3]benzazepin-5- yl)acetate | Starting from ethyl 2-(1-hydroxy-3- methoxy-7-methyl-6-oxo-7H-pyrido[4,3- d][3]benzazepin-5-yl)acetate NN423. LC- MS m/z [M + H]⁺: 371.1; purity: 98%. ¹H NMR (400 MHz, DMSO-d₆) δ 7.77 (dt, J = 7.9, 1.5 Hz, 1H), 7.47-7.21 (m, 3H), 5.82 (d, J = 1.3 Hz, 1H), 4.52-4.31 (m, 2H), 4.05-3.91 (m, 5H), 3.47-3.37 (m, 4H), 1.45 (d, J = 6.6 Hz, 3H), 1.01 (td, J = 7.1, 1.3 Hz, 3H). |
| NN426 | 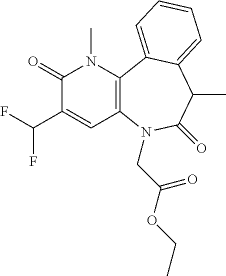 [SMILES: CCOC(=O)CN1C(=O)C(C)c2ccccc2-c2c1cc(C(F)F)c(=O)n2C] | Ethyl 2-[3- (difluoromethyl)- 1,7-dimethyl-2,6- dioxo-7H- pyrido[3,2- d][3]benzazepin-5- yl]acetate | Starting from ethyl 2-[3-(difluoromethyl)-2- hydroxy-7-methyl-6-oxo-7H-pyrido[3,2- d][3]benzazepin-5-yl]acetate NN425. LC- MS m/z [M + H]⁺: 391.0; purity: 83%. ¹H NMR (400 MHz, DMSO-d₆) δ 7.89 (s, 1H), 7.76 (d, J = 7.9, 1.3 Hz, 1H), 7.60 (t, J = 7.6, 1.3 Hz, 1H), 7.50-7.38 (m, 2H), 6.96 (t, J = 54.8 Hz, 1H), 4.49-4.14 (m, 2H), 3.98-3.88 (m, 2H), 3.81 (q, J = 6.7 Hz, 1H), 3.38 (s, 3H), 1.46 (d, J = 6.7 Hz, 3H), 1.01 (t, J = 7.1 Hz, 3H). |
| NN428 | 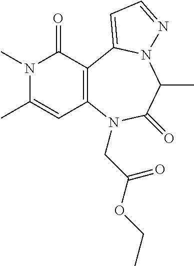 | Ethyl 2-(7,12,13- trimethyl-8,14- dioxo-5,6,9,13- tetrazatricyclo[8.4. 0.0^{2,6}]tetradeca- 1(10),2,4,11- tetraen-9- yl)acetate | Starting from ethyl 2-(14-hydroxy-7,12- dimethyl-8-oxo-5,6,9,13- tetrazatricyclo[8.4.0.0^{2,6}]tetradeca- 1(14),2,4,10,12-pentaen-9-yl)acetate NN427. LC-MS m/z [M + H]⁺: 345.2; purity: 95%. ¹H NMR (400 MHz, DMSO-d₆) δ 7.54 (d, J = 1.9 Hz, 1H), 6.72 (d, J = 1.9 Hz, 1H), 6.33 (s, 1H), 4.78 (q, J = 6.7 Hz, 1H), 4.48 (d, J = 17.5 Hz, 1H), 4.41 (d, J = 17.5 Hz, 1H), 4.04 (qd, J = 7.1, 2.0 Hz, 2H), 3.50 (s, 3H), 2.43 (s, 3H), 1.69 (d, J = 6.7 Hz, 3H), 1.09 (t, J = 7.1 Hz, 3H); |
| NN430 | 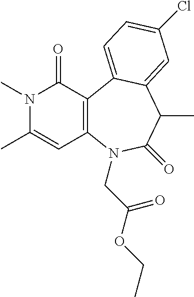 | Ethyl 2-(9-chloro- 2,3,7-trimethyl-1,6- dioxo-7H- pyrido[4,3- d][3]benzazepin-5- yl)acetate | Starting from ethyl 2-(9-chloro-3,7- dimethyl-1,6-dioxo-2,7-dihydropyrido[4,3- d][3]benzazepin-5-yl)acetate NN429. LC- MS m/z [M + H]⁺: 389.2, 391.2; purity: 99%. ¹H NMR (400 MHz, DMSO-d₆) δ 7.83 (d, J = 8.5 Hz, 1H), 7.38 (dd, J = 8.5, 2.2 Hz, 1H), 7.27 (d, J = 2.2 Hz, 1H), 6.35-6.25 (m, 1H), 4.47-4.28 (m, 2H), 3.98 (qt, J = 7.1, 3.6 Hz, 2H), 3.49 (s, 3H), 3.44 (d, J = 6.8 Hz, 1H), 2.42 (s, 3H), 1.44 (d, J = 6.8 Hz, 3H), 1.03 (t, J = 7.1 Hz, 3H). |
| NN431 | 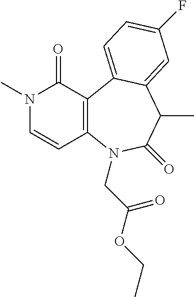 | Ethyl 2-(9-fluoro- 2,7-dimethyl-1,6- dioxo-7H- pyrido[4,3- d][3]benzazepin-5- yl)acetate | Starting from ethyl 2-(1-hydroxy-7-methyl- 6-oxo-7H-pyrido[4,3-d][3]benzazepin-5- yl)acetate NN401. LC-MS m/z [M + H]⁺: 359.2; purity: 100%. ¹H NMR (400 MHz, DMSO-d₆) δ 7.87 (dd, J = 8.8, 6.1 Hz, 1H), 7.80 (d, J = 7.6 Hz, 1H), 7.17 (td, J = 8.6, 2.7 Hz, 1H), 7.09 (dd, J = 10.2, 2.7 Hz, 1H), 6.34 (d, J = 7.5 Hz, 1H), 4.42 (d, J = 17.5 Hz, 1H), 4.33 (d, J = 17.5 Hz, 1H), 4.05-3.94 (m, 2H), 3.50 (s, 3H), 3.44 (q, J = 6.7 Hz, 1H), 1.44 (d, J = 6.8 Hz, 3H), 1.04 (t, J = 7.1 Hz, 3H). |
| NN433 | 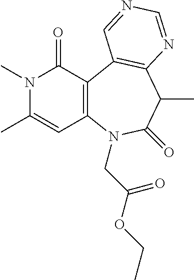 | Ethyl 2-(8,13,14- trimethyl-9,15- dioxo-4,6,10,14- tetrazatricyclo[9.4. 0.0^{2,7}]pentadeca- 1(11),2(7),3,5,12- pentaen-10- yl)acetate | Starting from ethyl 2-(8,13-dimethyl-9,15- dioxo-4,6,10,14- tetrazatricyclo[9.4.0.0^{2,7}]pentadeca- 1(11),2(7),3,5, 12-pentaen-10-yl)acetate NN432. LC-MS m/z [M + H]⁺: 357.2; purity: 92%. ¹H NMR (400 MHz, DMSO-d₆) δ 9.19 (s, 1H), 9.11 (s, 1H), 6.42 (d, J = 0.9 Hz, 1H), 4.51 (d, J = 17.5 Hz, 1H), 4.40 (d, J = 17.4 Hz, 1H), 3.97 (dd, J = 7.2, 3.3 Hz, 2H), 3.74-3.66 (m, 1H), 3.52 (s, 3H), 2.45 (s, 3H), 1.47 (d, J = 6.6 Hz, 3H), 1.00 (t, J = 7.1 Hz, 3H). |
| NN435 | 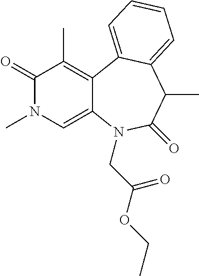 | Ethyl 2-(1,3,7- trimethyl-2,6- dioxo-7H- pyrido[3,4- d][3]benzazepin-5- yl)acetate | Starting from ethyl 2-(2-hydroxy-1,7- dimethyl-6-oxo-7H-pyrido[3,4- d][3]benzazepin-5-yl)acetate NN434. LC- MS m/z [M + H]⁺: 355.2; purity: 95%. ¹H NMR (400 MHz, DMSO-d₆) δ 7.81 (s, 1H), 7.51-7.44 (m, 2H), 7.42-7.38 (m, 1H), 7.33 (d, J = 7.8 Hz, 1H), 4.40 (d, J = 17.3 Hz, 1H), 4.21 (d, J = 17.2 Hz, 1H), 3.99- 3.86 (m, 2H), 3.76 (q, J = 6.7 Hz, 1H), 3.51 (s, 3H), 2.06 (s, 3H), 1.40 (d, J = 6.8 Hz, 3H), 1.01 (t, J = 7.1 Hz, 3H). |
| NN438 | 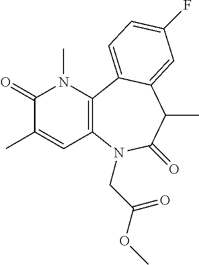 | Methyl 2-(9-fluoro- 1,3,7-trimethyl-2,6- dioxo-7H- pyrido[3,2- d][3]benzazepin-5- yl)acetate | Starting from ethyl 2-(9-fluoro-3,7- dimethyl-2,6-dioxo-1,7-dihydropyrido[3,2- d][3]benzazepin-5-yl)acetate NN437. LC- MS m/z [M + H]⁺: 359.2; purity: 83%. ¹H NMR (400 MHz, DMSO-d₆) δ 7.76 (dd, J = 8.7, 5.7 Hz, 1H), 7.51 (d, J = 1.4 Hz, 1H), 7.27 (td, J = 8.6, 2.6 Hz, 1H), 7.19 (dd, J = 10.2, 2.7 Hz, 1H), 4.41 (d, J = 17.4 Hz, 1H), 4.19 (d, J = 17.3 Hz, 1H), 3.74 (q, J = 6.5 Hz, 1H), 3.47 (s, 3H), 3.33 (s, 3H), 2.10 (s, 3H), 1.43 (d, J = 6.8 Hz, 3H). |
| NN443 | 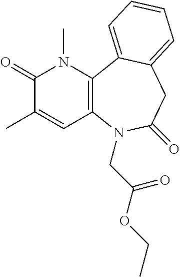 [SMILES: CCOC(=O)CN1C(=O)Cc2ccccc2-c2c1cc(C)c(=O)n2C] | Ethyl 2-(1,3- dimethyl-2,6- dioxo-7H- pyrido[3,2- d][3]benzazepin-5- yl)acetate | Starting from NN442. LC-MS (Method 5) m/z [M + H]⁺: 341.2; rt: 1.54 min; purity: 100%. |

### Intermediate NN445: 2-fluoro-6-methyl-pyridin-4-amine

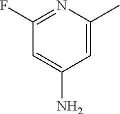

### Step 1: Synthesis of dimethyl 2-(4-amino-3,5-dichloro-6-fluoro-2-pyridyl)propanedioate

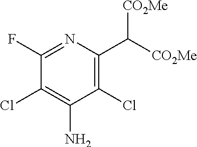 [SMILES: COC(=O)C(C(=O)OC)c1nc(F)c(Cl)c(N)c1Cl]

To a mixture of 4-amino-3,5-dichloro-2,6-difluoropyridine (402.2 g, 2021 mmol), dimethyl malonate (470 mL, 4030 mmol) and N,N-dimethylformamide (2.4 L) (colourless solution) was added potassium carbonate (830 g, 6005 mmol) (white suspension). The mixture was heated to 65° C. (internal temperature) for 19 hours. The mixture was cooled to ambient temperature and poured into mechanically stirred water (6 L). The solid was recovered on a sinter, washed with water (2 L) and dried trituration with TBME (2 L) and washing with isohexane (1.5 L) gave the title product (529 g, 84%) as a white crystalline solid. ¹H NMR (300 MHz, DMSO) δ 7.24 (s, 2H), 5.20 (s, 1H), 3.71 (s, 6H). ¹⁹F NMR (282 MHz, DMSO) δ−75.10. LC/MS m/z (method B9): 310.8, 312.8, 314.8 (M+H).

### Step 2: Synthesis of 3,5-dichloro-2-fluoro-6-methyl-pyridin-4-amine

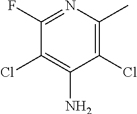

To dimethyl 2-(4-amino-3,5-dichloro-6-fluoro-2-pyridyl)propanedioate (370 g, 1190 mmol) and calcium chloride hexahydrate (780 g, 3560 mmol) was added 1-methyl-2-pyrrolidinone (1.5 L). The suspension was heated to 100° C. to give a solution. After 20 hours, analysis by LCMS showed 87% conversion to mono-ester. The temperature was increased to 125° C. After 72 hours, analysis by LCMS showed complete conversion to picoline. The mixture was cooled to 25° C. and poured into mechanically stirred water (2.5 L). The precipitate was recovered on a sinter, washed with water (1 L) and partially dried. The wet solid was suspended in ethanol (3 L) and heated to 65° C. to give a cloudy solution. Activated charcoal (30 g) was added and heating continued. After 15 minutes, the warm mixture was filtered through Celite. To the filtrate was added water (3 L, to give 1:1 ethanol:water). The mixture was then cooled to 5° C. for 20 hours. The crystals were recovered on a sinter, washed with water (600 mL) and dried (Note: solid is slightly volatile if dried at 50° C. at ~2 mbar) to give the title product as an off-white solid (179 g, 77%). ¹H NMR (300 MHz, DMSO) δ 6.94 (s, 2H), 2.35 (d, J=0.6 Hz, 3H). ¹⁹F NMR (282 MHz, DMSO) δ−76.01. LC/MS m/z (method B9): 194.8, 196.8, 198.8 (M+H), rt: 1.38 min, 100% purity.

### Step 3: Preparation of 2-fluoro-6-methyl-pyridin-4-amine

3,5-dichloro-2-fluoro-6-methyl-pyridin-4-amine (150 g, 769 mmol) and 5% palladium on charcoal (49 g, 23 mmol) were solubilised in methanol (2.6 L) and pyridine (125 mL). The mixture was placed under a 1 bar hydrogen atmosphere and heated to 50° C. for 18 hours. The mixture was then cooled, filtered through Celite and concentrated under reduced pressure. Water (750 mL) was added to the residue and the solution was extracted with ethyl acetate (3×750 mL). The organics were washed with brine (500 mL), dried and concentrated under reduced pressure to give the title product as a white solid (93 g, 91%). ¹H NMR (400 MHz, DMSO) δ 6.30 (s, 2H), 6.24 (s, 1H), 5.85 (s, 1H), 2.17 (t, J=1.5 Hz, 3H). ¹⁹F NMR (376 MHz, DMSO) δ−71.85. LC/MS (method B2) m/z (ES): 127 (M+H), rt: 2.1 min, 98.6% purity.

### IV. Examples

### General Procedure 1

To a solution of the corresponding intermediate of general formula (2) (1.0 eq.) in dry DMF (0.14 mol·L⁻¹) was added a 2-chloro-acetamide, such as 2-chloro-N-(4-trifluoromethylphenyl)acetamide (CAS 2707-23-5) (1.2 eq.), followed by K₂CO₃ (2.0 eq.) and potassium iodide (0.1 eq.) and the reaction mixture was stirred at room temperature for 16 h. The reaction mixture was diluted with EtOAc and washed with H₂O. The organic layer was separated, washed with brine, dried over Na₂SO₄ and concentrated under vacuum. The residue was purified by column chromatography on silica gel (using a gradient EtOAc in hexanes as eluent) and/or by reverse phase chromatography to afford the corresponding compound of the following examples.

### Example #1: N-(4-chloro-3-fluoro-phenyl)-2-(9,10-dimethyl-6-oxo-5-H-pyrido[3,2-d][1]benzazepin-7-yl)acetamide

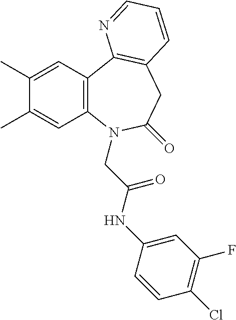 [SMILES: Cc1cc2c(cc1C)N(CC(=O)Nc1ccc(Cl)c(F)c1)C(=O)Cc1cccnc1-2]

The title product was prepared according to general procedure 1, starting from Intermediate N1 and 2-chloro-N-(4-chloro-3-fluoro-phenyl)acetamide (CAS 895641-02-8). Purification by preparative HPLC (basic elution) afforded the title compound as a white solid (6 mg, yield: 6%). LC-MS (Method B2) m/z [M+H]⁺: 424.1; rt: 4.57 min; purity: 98%. LC-MS (Method A2) m/z [M+H]⁺: 424.0; rt: 4.05 min; purity: 98%.

### Example #2: N-(4-chloro-3-fluoro-phenyl)-2-(9,10-dimethyl-6-oxo-5H-pyrido[4,3-d][1]benzazepin-7-yl)acetamide

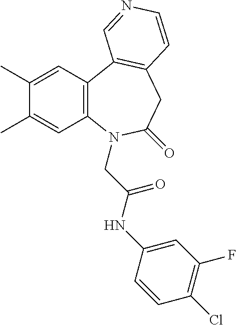 [SMILES: Cc1cc2c(cc1C)N(CC(=O)Nc1ccc(Cl)c(F)c1)C(=O)Cc1ccncc1-2]

The title product was prepared according to general procedure 1, starting from Intermediate N2 and 2-chloro-N-(4-chloro-3-fluoro-phenyl)acetamide (CAS 895641-02-8). Purification by preparative HPLC (basic elution) afforded the title compound as a white solid (7 mg, yield: 6%). LC-MS (Method B2) m/z [M+H]⁺: 424.1; rt: 4.47 min; purity: 98%. LC-MS (Method A2) m/z [M+H]⁺: 424.1; rt: 3.73 min; purity: 98%.

### Example #3: N-(4-chloro-3-fluoro-phenyl)-2-(9,10-dimethyl-6-oxo-5-H-pyrido[2,3-d][1]benzazepin-7-yl)acetamide

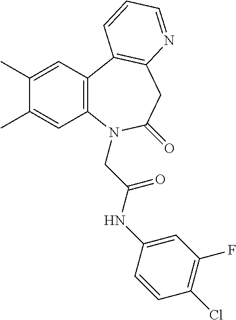 [SMILES: Cc1cc2c(cc1C)N(CC(=O)Nc1ccc(Cl)c(F)c1)C(=O)Cc1ncccc1-2]

The title product was prepared according to general procedure 1, starting from Intermediate N3 and 2-chloro-N-(4-chloro-3-fluoro-phenyl)acetamide (CAS 895641-02-8). Purification by preparative HPLC (basic elution) afforded the title compound as a white solid (4 mg, yield: 4%). LC-MS (Method B2) m/z [M+H]⁺: 424.1; rt: 4.57 min; purity: 98%. LC-MS (Method A2) m/z [M+H]⁺: 424.1; rt: 4.22 min; purity: 98%.

### Example #4: N-(4-chloro-3-fluoro-phenyl)-2-(3,9,10-trimethyl-6-oxo-5-H-pyrido[2,3-d][1]benzazepin-7-yl)acetamide

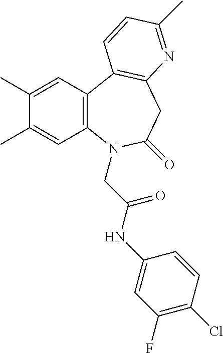 [SMILES: Cc1ccc2c(n1)CC(=O)N(CC(=O)Nc1ccc(Cl)c(F)c1)c1cc(C)c(C)cc1-2]

The title product was prepared according to general procedure 1, starting from Intermediate N4 and 2-chloro-N-(4-chloro-3-fluoro-phenyl)acetamide (CAS 895641-02-8). The residue was triturated in DCM to afford the title compound as a white solid (33 mg, yield: 38%). LC-MS (Method B2) m/z [M+H]⁺: 438.13; rt: 4.75 min; purity: 99%. LC-MS (Method A2) m/z [M+H]⁺: 438.12; rt: 3.97 min; purity: 98%. ¹H NMR (400 MHz, DMSO-d₆) δ 10.48 (s, 1H), 7.95 (d, J=7.9 Hz, 1H), 7.80-7.71 (m, 1H), 7.51 (t, J=8.7 Hz, 1H), 7.40-7.29 (m, 4H), 4.49 (d, J=16.6 Hz, 1H), 4.40 (d, J=16.6 Hz, 1H), 3.62 (d, J=12.2 Hz, 1H), 3.57 (d, J=12.2 Hz, 1H), 2.52 (s, 3H), 2.30 (s, 3H), 2.29 (s, 3H).

### Example #5: 2-(8,9-dimethyl-5-oxo-4H-pyrazolo[5,1-d][1,5]benzodiazepin-6-yl)-N-[4-(trifluoromethyl)phenyl]acetamide

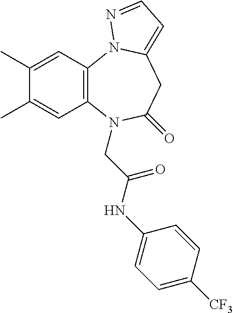 [SMILES: Cc1cc2c(cc1C)-n1nccc1CC(=O)N2CC(=O)Nc1ccc(C(F)(F)F)cc1]

The title product was prepared according to general procedure 1, starting from Intermediate N5 (50 mg, 0.22 mmol) and 2-chloro-N-(4-trifluoromethylphenyl)acetamide (CAS 2707-23-5). A white solid was isolated (57 mg, yield: 61%). LC-MS (Method B2) m/z [M+H]⁺: 429.1; rt: 4.55 min; purity>99%. LC-MS (Method A2) m/z [M+H]⁺: 429.1; rt: 4.75 min; purity: >99%. ¹H NMR (400 MHz, DMSO-d₆) δ 10.58 (s, 1H), 7.81 (d, J=8.5 Hz, 2H), 7.76 (d, J=1.8 Hz, 1H), 7.69 (d, J=8.5 Hz, 2H), 7.54 (s, 1H), 7.36 (s, 1H), 6.42 (d, J=1.8 Hz, 1H), 4.58 (d, J=16.7 Hz, 1H), 4.44 (d, J=16.7 Hz, 1H), 3.79 (d, J=14.2 Hz, 1H), 3.54 (d, J=14.2 Hz, 1H), 2.30 (s, 3H), 2.29 (s, 3H).

### Example #6: 2-(5-methyl-9-oxo-4,8,14-triazatricyclo[9.4.0.0^{2,7}]pentadeca-1(11),2(7),3,5,12,14-hexaen-8-yl)-N-[4-(trifluoromethyl)phenyl]acetamide

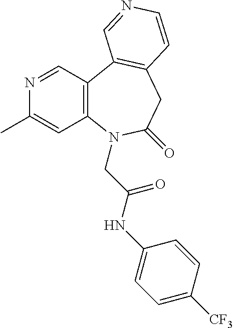 [SMILES: Cc1cc2c(cn1)-c1cnccc1CC(=O)N2CC(=O)Nc1ccc(C(F)(F)F)cc1]

Under inert atmosphere, to a solution of example #11 (105 mg, 0.235 mmol) in dry THF (2.4 mL) were added a 2 M solution of methylzinc chloride in THF (0.47 mL, 0.94 mmol), cuprous iodide (5 mg, 0.026 mmol) and 1,1′-bis(diphenylphosphino)ferrocene-palladium(II)dichloride dichloromethane complex (97 mg, 0.118 mmol) and the reaction mixture was stirred at 70° C. for 4 h. After cooling to room temperature, the reaction mixture was partitioned between EtOAc and water. The phases were separated and the aqueous layer was extracted twice with EtOAc. The organic layer was dried over MgSO₄, filtered off and concentrated under vacuum. The residue was purified by column chromatography on silica gel (using a gradient of 0% to 5% MeOH in DCM as eluent) and triturated in DCM to afford the title compound (38 mg, yield: 38%) as a white solid. LC-MS (Method B2) m/z [M+H]⁺: 427.1; rt: 3.80 min; purity>99%. LC-MS (Method A2) m/z [M+H]⁺: 427.1; rt: 3.29 min; purity: 98%. ¹H NMR (400 MHz, DMSO-d₆) δ 10.62 (s, 1H), 8.93 (s, 1H), 8.78 (s, 1H), 8.65 (d, J=5.0 Hz, 1H), 7.79 (d, J=8.5 Hz, 2H), 7.68 (d, J=8.5 Hz, 2H), 7.54 (d, J=5.0 Hz, 1H), 7.37 (s, 1H), 4.70-4.47 (m, 2H), 3.76-3.52 (m, 2H), 2.56 (s, 3H).

### Example #7: 2-(13-chloro-9-oxo-3,10,14-triazatricyclo[9.4.0.0^{2,7}]pentadeca-1(11),2(7),3,5,12,14-hexaen-10-yl)-N-[4-(trifluoromethyl)phenyl]acetamide

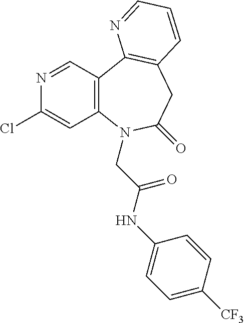 [SMILES: O=C(CN1C(=O)Cc2cccnc2-c2cnc(Cl)cc21)Nc1ccc(C(F)(F)F)cc1]

The title product was prepared according to general procedure 1, starting from Intermediate N6 (150 mg, 0.61 mmol) and 2-chloro-N-(4-trifluoromethylphenyl)acetamide (CAS 2707-23-5). The reaction mixture was stirred at 60° C. overnight. A white solid was isolated (160 mg, yield: 59%). LC-MS (Method B2) m/z [M+H]⁺: 447.0; rt: 4.10 min; purity>99%. LC-MS (Method A2) m/z [M+H]⁺: 447.0; rt: 4.42 min; purity>99%. ¹H NMR (400 MHz, DMSO-d₆) δ 10.68 (s, 1H), 8.89 (s, 1H), 8.74 (dd, J=4.8, 1.6 Hz, 1H), 7.96 (dd, J=7.8, 1.6 Hz, 1H), 7.80 (d, J=8.6 Hz, 2H), 7.69 (d, J=8.6 Hz, 2H), 7.61 (s, 1H), 7.56 (dd, J=7.8, 4.8 Hz, 1H), 4.66 (s, 2H), 3.81-3.60 (m, 2H).

### Example #8: 2-(8,9-dimethyl-5-oxo-4H-imidazo[1,2-a][1,5]benzodiazepin-6-yl)-N-[4-(trifluoromethyl)phenyl]acetamide

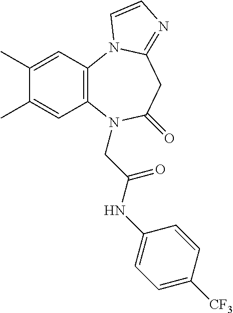 [SMILES: Cc1cc2c(cc1C)-n1ccnc1CC(=O)N2CC(=O)Nc1ccc(C(F)(F)F)cc1]

The title product was prepared according to general procedure 1, starting from Intermediate N7 (50 mg, 0.22 mmol) and 2-chloro-N-(4-trifluoromethylphenyl)acetamide (CAS 2707-23-5). A white solid was isolated (35 mg, yield: 37%). LC-MS (Method B2) m/z [M+H]⁺: 429.1; rt: 4.17 min; purity>99%. LC-MS (Method A2) m/z [M+H]⁺: 429.1; rt: 3.65 min; purity: 99%. ¹H NMR (400 MHz, DMSO-d₆) δ 10.57 (s, 1H), 7.79 (d, J=8.5 Hz, 2H), 7.74-7.64 (m, 3H), 7.41-7.39 (m, 2H), 7.06 (d, J=1.4 Hz, 1H), 4.63-4.45 (m, 2H), 3.71-3.55 (m, 2H), 2.30 (s, 3H), 2.27 (s, 3H).

### Example #9: 2-(9,10-dimethyl-6-oxo-5H-pyrimido[5,4-d][1]benzazepin-7-yl)-N-[4-(trifluoromethyl)phenyl]acetamide

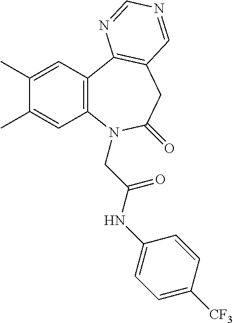 [SMILES: Cc1cc2c(cc1C)N(CC(=O)Nc1ccc(C(F)(F)F)cc1)C(=O)Cc1cncnc1-2]

The title product was prepared according to general procedure 1, starting from Intermediate N8 (50 mg, 0.21 mmol) and 2-chloro-N-(4-trifluoromethylphenyl)acetamide (CAS 2707-23-5). The reaction mixture was stirred at 60° C. overnight. A white solid was isolated (29 mg, yield: 32%). LC-MS (Method B2) m/z [M+H]⁺: 441.1; rt: 4.42 min; purity>99%. LC-MS (Method A2) m/z [M+H]⁺: 441.0; rt: 4.58 min; purity: 99%. ¹H NMR (400 MHz, DMSO-d₆) δ 10.53 (s, 1H), 9.25 (s, 1H), 8.86 (s, 1H), 7.80-7.71 (m, 3H), 7.67 (d, J=8.7 Hz, 2H), 7.37 (s, 1H), 4.53 (s, 2H), 3.69 (d, J=13.2 Hz, 1H), 3.48 (d, J=13.2 Hz, 1H), 2.32 (s, 6H).

### Example #10: 2-(2-chloro-9,10-dimethyl-6-oxo-5H-pyrido[3,2-d][1]benzazepin-7-yl)-N-[4-(trifluoromethyl)phenyl]acetamide

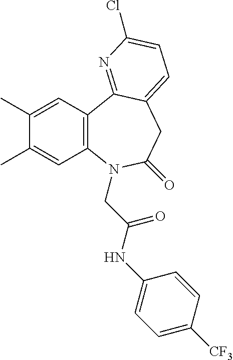 [SMILES: Cc1cc2c(cc1C)N(CC(=O)Nc1ccc(C(F)(F)F)cc1)C(=O)Cc1ccc(Cl)nc1-2]

The title product was prepared according to general procedure 1, starting from Intermediate N9 (118 mg, 0.43 mmol) and 2-chloro-N-(4-trifluoromethylphenyl)acetamide (CAS 2707-23-5). The reaction mixture was stirred at 60° C. overnight. A white solid was isolated (93 mg, yield: 45%). LC-MS (Method B2) m/z [M+H]⁺: 474.0; rt: 5.11 min; purity>99%. LC-MS (Method A2) m/z [M+H]⁺: 474.0; rt: 5.27 min; purity: 97%. ¹H NMR (400 MHz, DMSO-d₆) δ 10.70 (s, 1H), 7.94 (d, J=8.0 Hz, 1H), 7.78 (d, J=8.5 Hz, 2H), 7.67 (d, J=8.5 Hz, 2H), 7.61 (s, 1H), 7.56 (d, J=8.0 Hz, 1H), 7.33 (s, 1H), 4.55 (d, J=16.7 Hz, 1H), 4.47 (d, J=16.7 Hz, 1H), 3.66 (d, J=12.9 Hz, 1H), 3.41 (d, J=12.9 Hz, 1H), 2.32 (s, 3H), 2.31 (s, 3H).

### Example #11: 2-(5-chloro-9-oxo-4,8,14-triazatricyclo[9.4.0.0^{2,7}]pentadeca-1(11),2(7),3,5,12,14-hexaen-8-yl)-N-[4-(trifluoromethyl)phenyl]acetamide

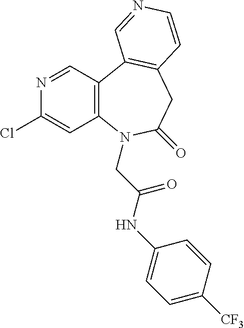 [SMILES: O=C(CN1C(=O)Cc2ccncc2-c2cnc(Cl)cc21)Nc1ccc(C(F)(F)F)cc1]

The title product was prepared according to general procedure 1, starting from Intermediate N10 (150 mg, 0.61 mmol) and 2-chloro-N-(4-trifluoromethylphenyl)acetamide (CAS 2707-23-5). The residue was triturated in a 1:1 mixture of DCM/Et₂O to give the title product (126 mg, yield: 46%) as a white solid. LC-MS (Method B2) m/z [M+H]⁺: 447.0; rt: 4.13 min; purity: 95%. LC-MS (Method A2) m/z [M+H]⁺: 447.0; rt: 3.82 min; purity: 95%. ¹H NMR (400 MHz, DMSO-d₆) δ 10.67 (s, 1H), 8.96 (s, 1H), 8.79 (s, 1H), 8.69 (d, J=5.0 Hz, 1H), 7.79 (d, J=8.5 Hz, 2H), 7.69 (d, J=8.5 Hz, 2H), 7.57 (d, J=5.0 Hz, 1H), 4.65 (s, 2H), 3.76-3.68 (m, 2H).

### Example #12: 2-(13-methyl-9-oxo-3,10,14-triazatricyclo[9.4.0.0^{2,7}]pentadeca-1(11),2(7),3,5,12,14-hexaen-10-yl)-N-[4-(trifluoromethyl)phenyl]acetamide

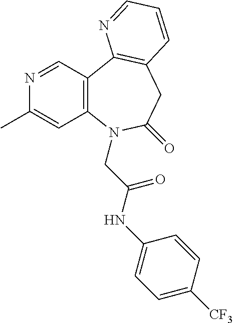 [SMILES: Cc1cc2c(cn1)-c1ncccc1CC(=O)N2CC(=O)Nc1ccc(C(F)(F)F)cc1]

The title product was obtained following the same procedure as the one described for the preparation of Example #6 but starting from Example #7. The reaction mixture was heated at 60° C. for 1 h. Purification by preparative HPLC (acidic elution) afforded the title product as a white solid (71 mg, yield: 50%). LC-MS (Method B2) m/z [M+H]⁺: 427.0; rt: 3.90 min; purity>99%. LC-MS (Method A2) m/z [M+H]⁺: 447.0; rt: 3.46 min; purity: 98%. ¹H NMR (400 MHz, DMSO-d₆) δ 10.55 (s, 1H), 9.01 (s, 1H), 8.72 (d, J=4.8 Hz, 1H), 7.92 (d, J=7.6 Hz, 1H), 7.80 (d, J=8.5 Hz, 2H), 7.69 (d, J=8.5 Hz, 2H), 7.51 (dd, J=7.6, 4.8 Hz, 1H), 7.32 (s, 1H), 4.65 (d, J=16.8 Hz, 2H), 4.63 (d, J=16.8 Hz, 2H), 3.71 (d, J=13.1 Hz, 2H), 3.56 (d, J=13.1 Hz, 2H), 2.56 (s, 3H).

### Example #13: 2-(3-chloro-6-oxo-7H-pyrido[4,3-d][3]benzazepin-5-yl)-N-[4-(trifluoromethyl)phenyl]acetamide

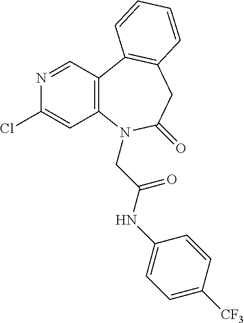 [SMILES: O=C(CN1C(=O)Cc2ccccc2-c2cnc(Cl)cc21)Nc1ccc(C(F)(F)F)cc1]

The title product was prepared according to general procedure 1, starting from Intermediate N11 (30 mg, 0.12 mmol) and 2-chloro-N-(4-trifluoromethylphenyl)acetamide (CAS 2707-23-5). A beige solid was isolated (18 mg, yield: 33%). LC-MS (Method B2) m/z [M+H]⁺: 446.05; rt: 4.63 min; purity: 95%. ¹H NMR (400 MHz, DMSO-d₆) δ 10.65 (s, 1H), 8.67 (s, 1H), 7.80 (d, J=8.3 Hz, 2H), 7.77-7.76 (m, 1H), 7.69 (d, J=8.3 Hz, 2H), 7.59 (s, 1H), 7.52-7.48 (m, 3H), 4.63 (s, 2H), 3.66 (d, J=12.8 Hz, 1H), 3.56 (d, J=12.8 Hz, 1H).

The following Examples #14-17 have been prepared according to general procedure 1, from commercially available 2-chloro-N-(4-trifluoromethylphenyl)acetamide and other specific intermediates referenced in the table below.

| Ex | Structure Example | Example Name | Protocolumn and characterization |
|---|---|---|---|
| #14 | 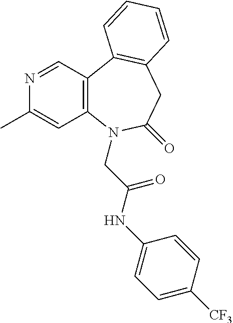 [SMILES: Cc1cc2c(cn1)-c1ccccc1CC(=O)N2CC(=O)Nc1ccc(C(F)(F)F)cc1] | 2-(3-methyl-6-oxo- 7H-pyrido[4,3-d][3] benzazepin-5-yl)-N- [4-(trifluoromethyl)- phenyl]acetamide | Starting from Intermediate N79 LC-MS (Method B2) m/z [M + H]⁺: 426.06; rt: 4.31 min; purity: 97%. LC-MS (Method A2) m/z [M + H]⁺: 426.01; rt: 3.65 min; purity: 98%. ¹H NMR (400 MHz, DMSO- d₆) δ 10.62 (s, 1H), 8.68 (s, 1H), 7.81 (d, J = 8.3 Hz, 2H), 7.74-7.68 (m, 3H), 7.50- 7.48 (m, 3H), 7.31 (s, 1H), 4.62 (d, J = 16.9 Hz, 1H), 4.49 (d, J = 16.9 Hz, 1H), 3.63 (d, J = 12.7 Hz, 1H), 3.47 (d, J = 12.7 Hz, 1H), 2.54 (s, 3H). |
| #15 | 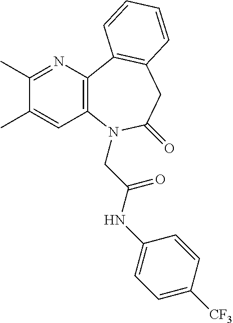 [SMILES: Cc1cc2c(nc1C)-c1ccccc1CC(=O)N2CC(=O)Nc1ccc(C(F)(F)F)cc1] | 2-(2,3-dimethyl-6- oxo-7H-pyrido[3,2- d][3]benzazepin-5- yl)-N-[4-(trifluoro- methyl)phenyl] acetamide | Starting from Intermediate N80. LC-MS (Method B1) m/z [M + H]⁺: 440.04; rt: 2.81 min; purity: 96%. ¹H NMR (400 MHz, DMSO-d₆) δ 10.75 (s, 1H), 7.95 (s, 1H), 7.81 (d, J = 8.3 Hz, 2H), 7.69-7.67 (m, 3H), 7.47-7.41 (m, 3H), 4.55 (d, J = 16.8 Hz, 1H), 4.40 (d, J = 16.8 Hz, 1H), 3.58 (d, J = 12.8 Hz, 1H), 3.44 (d, J = 12.8 Hz, 1H), 2.50 (s, 3H), 2.33 (s, 3H). |
| #16 | 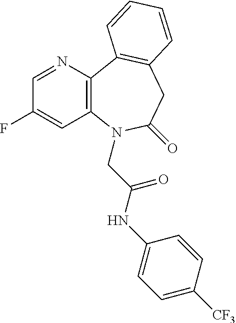 [SMILES: O=C(CN1C(=O)Cc2ccccc2-c2ncc(F)cc21)Nc1ccc(C(F)(F)F)cc1] | 2-(3-fluoro-6-oxo- 7H-pyrido[3,2-d] [3]benzazepin-5- yl)-N-[4-(trifluoro- methyl)phenyl] acetamide | Starting from Intermediate N81. LC-MS (Method B2) m/z [M + H]⁺: 430; rt: 4.56 min; purity: 98%. LC-MS (Method A2) m/z [M + H]⁺: 429.99; rt: 4.75 min; purity: 98%. ¹H NMR (400 MHz, DMSO-d₆) δ 10.65 (s, 1H), 8.66 (s, 1H), 7.94-7.90 (m, 2H), 7.80 (d, J = 8.3 Hz, 2H), 7.69 (d, J = 8.3 Hz, 2H) 7.52-7.44 (m, 3H), 4.58 (d, J = 16.8 Hz, 1H), 4.54 (d, J = 16.8 Hz, 1H), 3.64 (d, J = 12.8 Hz, 1H), 3.56 (d, J = 12.8 Hz, 1H). |
| #17 | 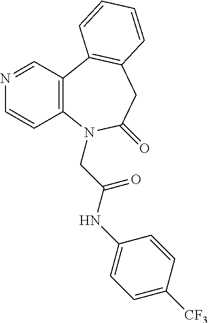 [SMILES: O=C(CN1C(=O)Cc2ccccc2-c2cnccc21)Nc1ccc(C(F)(F)F)cc1] | 2-(6-oxo-7H-pyrido [4,3-d][3]benzazepin- 5-yl)-N-[4-(trifluoro- methyl)phenyl] acetamide | Starting from Intermediate N82 . LC-MS (Method B2) m/z [M + H]⁺: 412.02; rt: 4.16 min; purity: 99%. LC-MS (Method A2) m/z [M + H]⁺: 412.00; rt: 3.71 min; purity: 99%. ¹H NMR (400 MHz, DMSO- d₆) δ 10.64 (s, 1H), 8.83 (s, 1H), 8.61 (d, J = 5.5 Hz, 1H), 7.81-7.76 (m, 3H), 7.69 (d, J = 8.4 Hz, 2H), 7.52-7.43 (m, 4H), 4.67 (d, J = 16.8 Hz, 1H), 4.50 (d, J = 16.8 Hz, 1H), 3.66 (d, J = 12.8 Hz, 1H), 3.49 (d, J = 12.8 Hz, 1H). |

### Example #18: N-(3-chloro-4-methyl-phenyl)-2-(13-chloro-9-oxo-3,10,14-triazatricyclo[9.4.0.0^{2,7}]pentadeca-1(11),2(7),3,5,12,14-hexaen-10-yl)acetamide

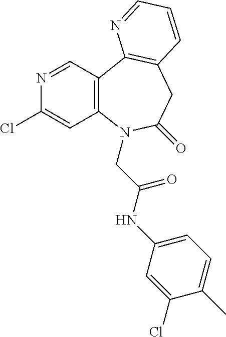 [SMILES: Cc1ccc(NC(=O)CN2C(=O)Cc3cccnc3-c3cnc(Cl)cc32)cc1Cl]

The title product was prepared according to general procedure 1, starting from Intermediate N6 and 2-chloro-N-(3-chloro-4-methylphenyl)acetamide (CAS 99585-97-4). LC-MS (Method B2) m/z [M+H]⁺: 427.13; rt: 4.27 min; purity: 96%. LC-MS (Method A2) m/z [M+H]⁺: 427.08; rt: 4.45 min; purity: 96%. ¹H NMR (400 MHz, DMSO-d₆) δ 10.41 (s, 1H), 8.88 (s, 1H), 8.74 (d, J=4.6 Hz, 1H), 7.95 (d, J=7.7 Hz, 1H), 7.79 (s, 1H), 7.60-7.53 (m, 2H), 7.34 (d, J=8.3 Hz, 1H), 7.29 (d, J=8.3 Hz, 1H), 4.62 (s, 2H), 3.75 (d, J=12.8 Hz, 1H), 3.65 (d, J=12.8 Hz, 1H), 2.28 (s, 3H).

### Example #19: N-(4-bromo-3-chloro-phenyl)-2-(13-chloro-9-oxo-3,10,14-triazatricyclo[9.4.0.0^{2,7}]pentadeca-1(11),2(7),3,5,12,14-hexaen-10-yl)acetamide

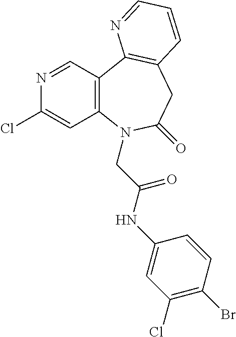 [SMILES: O=C(CN1C(=O)Cc2cccnc2-c2cnc(Cl)cc21)Nc1ccc(Br)c(Cl)c1]

The title product was prepared according to general procedure 1, starting from Intermediate N6 and 2-chloro-N-(4-bromo-3-chlorophenyl)acetamide (CAS: 22521-43-3). LC-MS (Method A1) m/z [M+H]⁺: 490.96; rt: 2.6 min; purity>99%.

### Example #20: N-(4-chloro-3-fluoro-phenyl)-2-(13-chloro-9-oxo-3,10,14-triazatricyclo[9.4.0.0^{2,7}]pentadeca-1(11),2(7),3,5,12,14-hexaen-10-yl)acetamide

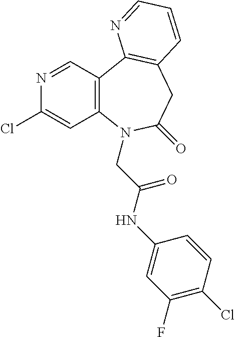 [SMILES: O=C(CN1C(=O)Cc2cccnc2-c2cnc(Cl)cc21)Nc1ccc(Cl)c(F)c1]

The title product was prepared according to general procedure 1, starting from Intermediate N6 and 2-chloro-N-(4-chloro-3-fluorophenyl)acetamide (CAS 895641-02-8). LC-MS (Method A1) m/z [M+H]⁺: 431.04; rt: 2.45 min; purity>99%.

### Example #21: N-[4-(trifluoromethyl)phenyl]-2-(3,9,10-trimethyl-6-oxo-5H-pyrido[4,3-d][1]benzazepin-7-yl)acetamide

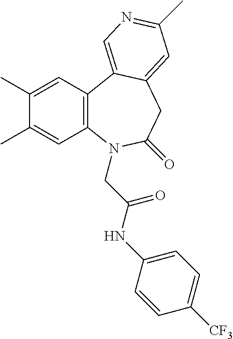 [SMILES: Cc1cc2c(cn1)-c1cc(C)c(C)cc1N(CC(=O)Nc1ccc(C(F)(F)F)cc1)C(=O)C2]

The title product was prepared according to general procedure 1 starting from Intermediate N130 and 2-chloro-N-(4-trifluoromethylphenyl)acetamide (CAS 2707-23-5). LC-MS (Method B2) m/z [M+H]⁺: 454.20; rt: 4.62 min; purity>99%. LC-MS (Method A2) m/z [M+H]⁺: 454.14; rt: 3.76 min; purity>99%. ¹H NMR (400 MHz, DMSO-d₆) δ 10.51 (s, 1H), 8.69 (s, 1H), 7.78 (d, J=8.3 Hz, 2H), 7.67 (d, J=8.3 Hz, 2H), 7.45 (s, 1H), 7.32 (s, 1H), 4.50 (d, J=16.7 Hz, 1H), 4.41 (d, J=16.7 Hz, 1H), 3.51 (d, J=12.3 Hz, 1H), 3.43 (d, J=12.3 Hz, 1H), 2.52 (s, 3H), 2.31 (s, 3H), 2.29 (s, 3H).

### Example #22: 2-(9,10-dimethyl-6-oxo-5H-pyrido[3,2-d][1]benzazepin-7-yl)-N-[4-(trifluoromethyl)phenyl]acetamide

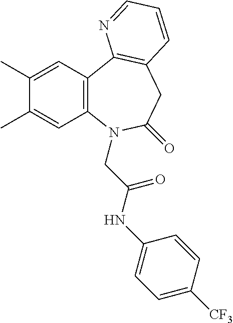 [SMILES: Cc1cc2c(cc1C)N(CC(=O)Nc1ccc(C(F)(F)F)cc1)C(=O)Cc1cccnc1-2]

The title product was prepared according to general procedure 1, starting from Intermediate N1 and 2-chloro-N-(4-trifluoromethylphenyl)acetamide (CAS 2707-23-5). LC-MS (Method B2) m/z [M+H]⁺: 440.04; rt: 4.64 min; purity>99%. LC-MS (Method A2) m/z [M+H]⁺: 440.06; rt: 4.12 min; purity: 98%. ¹H NMR (400 MHz, DMSO-d₆) δ 10.65 (s, 1H), 8.67 (d, J=4.7 Hz, 1H), 7.84 (d, J=6.8 Hz, 1H), 7.80 (d, J=8.3 Hz, 2H), 7.69-7.66 (m, 3H), 7.43 (dd, J=6.8, 4.7 Hz, 1H), 7.30 (s, 1H), 4.53 (d, J=16.7 Hz, 1H), 4.41 (d, J=16.7 Hz, 1H), 3.59 (d, J=12.8 Hz, 1H), 3.44 (d, J=12.8 Hz, 1H), 2.31 (s, 3H), 2.30 (s, 3H).

### Example #23: 2-(9,10-dimethyl-6-oxo-5H-pyrido[2,3-d][1]benzazepin-7-yl)-N-[4-(trifluoromethyl)phenyl]acetamide

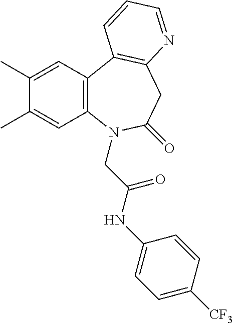 [SMILES: Cc1cc2c(cc1C)N(CC(=O)Nc1ccc(C(F)(F)F)cc1)C(=O)Cc1ncccc1-2]

The title product was prepared according to general procedure 1, starting from Intermediate N3 and 2-chloro-N-(4-trifluoromethylphenyl)acetamide (CAS 2707-23-5). LC-MS (Method B2) m/z [M+H]⁺: 440.08; rt: 4.64 min; purity>99%. LC-MS (Method A2) m/z [M+H]⁺: 440.02; rt: 4.27 min; purity>99%. ¹H NMR (400 MHz, DMSO-d₆) δ 10.63 (s, 1H), 8.55 (d, J=6.2 Hz, 1H), 8.08 (d, J=7.8 Hz, 1H), 7.78 (d, J=8.4 Hz, 2H), 7.67 (d, J=8.4 Hz, 2H), 7.50 (dd, J=7.8, 6.2 Hz, 1H), 7.42 (s, 1H), 7.35 (s, 1H), 4.54 (d, J=16.6 Hz, 1H), 4.46 (d, J=16.6 Hz, 1H), 3.69 (d, J=12.8 Hz, 1H), 3.64 (d, J=12.8 Hz, 1H), 2.31 (s, 3H), 2.29 (s, 3H).

### Example #24: 2-(9,10-dimethyl-6-oxo-5H-pyrido[4,3-d][1]benzazepin-7-yl)-N-[4-(trifluoromethyl)phenyl]acetamide

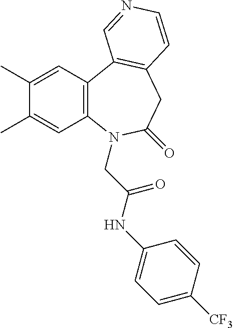 [SMILES: Cc1cc2c(cc1C)N(CC(=O)Nc1ccc(C(F)(F)F)cc1)C(=O)Cc1ccncc1-2]

The title product was prepared according to general procedure 1, starting from Intermediate N₂ and 2-chloro-N-(4-trifluoromethylphenyl)acetamide (CAS 2707-23-5). LC-MS (Method B4) m/z [M+H]⁺: 440.16; rt: 4.17 min; purity: 94%. LC-MS (Method A3) m/z [M+H]⁺: 440.16; rt: 4.46 min; purity: 94%. ¹H NMR (400 MHz, DMSO-d₆) δ 10.53 (s, 1H), 8.84 (s, 1H), 8.58 (d, J=4.9 Hz, 1H), 7.78 (d, J=8.6 Hz, 2H), 7.67 (d, J=8.6 Hz, 2H), 7.49-7.47 (m, 2H), 7.35 (s, 1H), 4.51 (d, J=16.7 Hz, 1H), 4.43 (d, J=16.7 Hz, 1H), 3.60 (d, J=12.5 Hz, 1H), 3.46 (d, J=12.5 Hz, 1H), 2.32 (s, 3H), 2.30 (s, 3H).

### Example #25: 2-(5-chloro-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

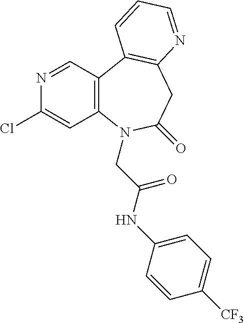 [SMILES: O=C(CN1C(=O)Cc2ncccc2-c2cnc(Cl)cc21)Nc1ccc(C(F)(F)F)cc1]

The title product was prepared according to general procedure 1, starting from Intermediate N12 (8 mg, 0.03 mmol) and 2-chloro-N-(4-trifluoromethylphenyl)acetamide (CAS 2707-23-5). Purification by preparative HPLC (basic elution) afforded the title product (2 mg, yield: 16%) as a white solid. LC-MS (Method B2) m/z [M+H]⁺: 447.08; rt: 3.91 min; purity: 93%. LC-MS (Method A2) m/z [M+H]⁺: 447.08; rt: 4.29 min; purity: 92%. ¹H NMR (400 MHz, DMSO-d₆) δ 10.67 (s, 1H), 8.68 (s, 1H), 8.66 (d, J=4.8 Hz, 1H), 8.23 (s, J=7.9 Hz, 1H), 7.78 (d, J=8.3 Hz, 2H), 7.70-7.65 (m, 3H), 7.58 (dd, 7.9, 4.8 Hz, 1H), 4.67 (d, J=16.8 Hz, 1H), 4.66 (d, J=16.8 Hz, 1H), 3.88 (d, J=12.7 Hz, 1H), 3.77 (d, J=12.7 Hz, 1H).

### Example #26: 2-(12-chloro-8-oxo-5-thia-9,13-diazatricyclo[8.4.0.0^{2,6}]tetradeca-1(10),2(6),3,11,13-pentaen-9-yl)-N-[4-(trifluoromethyl)phenyl]acetamide

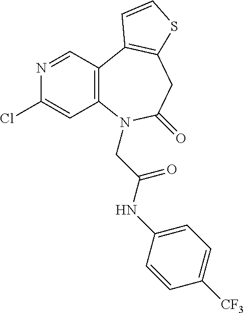 [SMILES: O=C(CN1C(=O)Cc2sccc2-c2cnc(Cl)cc21)Nc1ccc(C(F)(F)F)cc1]

The title product was prepared according to general procedure 1, starting from Intermediate N13 (20 mg, 0.08 mmol) and 2-chloro-N-(4-trifluoromethylphenyl)acetamide (CAS 2707-23-5). The residue was triturated in a mixture of DCM/Et₂O 50/50 to give the title product (23 mg, yield: 60%) as a white solid. LC-MS (Method B2) m/z [M+H]⁺: 451.91; rt: 4.63 min; purity: 96%. LC-MS (Method A2) m/z [M+H]⁺: 451.87; rt: 4.83 min; purity: 94%. ¹H NMR (400 MHz, DMSO-d₆) δ 10.69 (s, 1H), 8.74 (s, 1H), 7.82 (d, J=8.3 Hz, 2H), 7.70 (d, J=8.3 Hz, 2H), 7.64-7.57 (m, 3H), 4.64 (bs, 2H), 3.89 (bs, 1H), 3.58 (bs, 1H).

### Example #27: 2-(1,3-dimethyl-6-oxo-7H-pyrido[4,3-d][3]benzazepin-5-yl)-N-[4-(trifluoromethyl)phenyl]acetamide

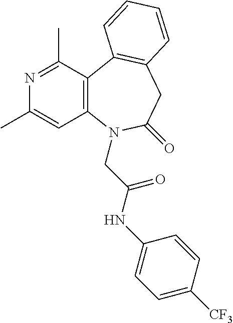 [SMILES: Cc1cc2c(c(C)n1)-c1ccccc1CC(=O)N2CC(=O)Nc1ccc(C(F)(F)F)cc1]

The title product was prepared according to general procedure 1, starting from Intermediate N14 (8 mg, 0.03 mmol) and 2-chloro-N-(4-trifluoromethylphenyl)acetamide (CAS 2707-23-5). A white solid was isolated (2 mg, yield: 16%). LC-MS (Method B2) m/z [M+H]⁺: 440.04; rt: 4.39 min; purity: 95%. LC-MS (Method A2) m/z [M+H]⁺: 440.21; rt: 3.64 min; purity: 90%. The following Examples #28-35 have been prepared according to general procedure 1, starting from commercially available 2-chloro-N-(4-trifluoromethylphenyl)acetamide (CAS 2707-23-5) and other specific intermediates referenced in the table below.

| Ex | Structure Example | Example Name | Protocolumn and characterization |
|---|---|---|---|
| #28 | 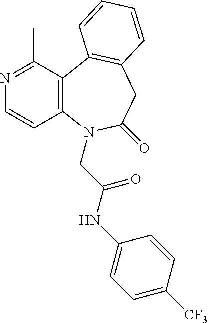 [SMILES: Cc1nccc2c1-c1ccccc1CC(=O)N2CC(=O)Nc1ccc(C(F)(F)F)cc1] | 2-(1-methyl-6-oxo- 7H-pyrido[4,3-d] [3]benzazepin-5- yl)-N-[4-(trifluoro- methyl)phenyl] acetamide | Starting from Intermediate N83. LC-MS (Method B2) m/z [M + H]⁺: 426.03; rt: 4.26 min; purity: 83%. LC-MS (Method A2) m/z [M + H]⁺: 426.01; rt: 3.61 min; purity: 81%. |
| #29 | 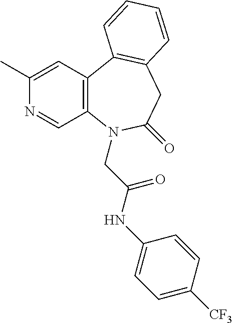 [SMILES: Cc1cc2c(cn1)N(CC(=O)Nc1ccc(C(F)(F)F)cc1)C(=O)Cc1ccccc1-2] | 2-(2-methyl-6-oxo- 7H-pyrido[3,4-d] [3]benzazepin-5- yl)-N-[4-(trifluoro- methyl)phenyl] acetamide | Starting from Intermediate N84. LC-MS (Method B2) m/z [M + H]⁺: 426.06; rt: 4.34 min; purity: 97%. LC-MS (Method A2) m/z [M + H]⁺: 426.04; rt: 3.75 min; purity: 97%. ¹H NMR (400 MHz, DMSO-d₆) δ 10.58 (s, 1H), 8.64 (s, 1H), 7.80-7.77 (m, 3H), 7.69 (d, J = 8.4 Hz, 2H), 7.56-7.46 (m, 4H), 4.61 (d, J = 16.8 Hz, 1H), 4.54 (d, J = 16.8 Hz, 1H), 3.60 (d, J = 12.8 Hz, 1H), 3.47 (d, J = 12.8 Hz, 1H), 2.56 (s, 3H). |
| #30 | 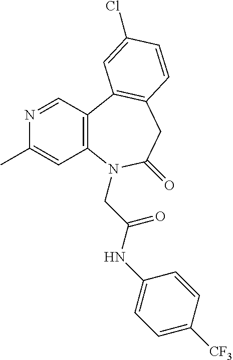 [SMILES: Cc1cc2c(cn1)-c1cc(Cl)ccc1CC(=O)N2CC(=O)Nc1ccc(C(F)(F)F)cc1] | 2-(10-chloro-3- methyl-6-oxo-7H- pyrido[4,3-d][3] benzazepin-5-yl)- N-[4-(trifluoro- methyl)phenyl] acetamide | Starting from Intermediate N85. LC-MS (Method B2) m/z [M + H]⁺: 459.97; rt: 4.58 min; purity >99%. LC-MS (Method A2) m/z [M + H]⁺: 459.96; rt: 3.89 min; purity: 98%. ¹H NMR (400 MHz, DMSO-d₆) δ 10.69 (s, 1H), 8.71 (s, 1H), 7.81-7.79 (m, 3H), 7.69 (d, J = 8.7 Hz, 2H), 7.55-7.49 (m, 2H), 7.34 (s, 1H), 4.61 (d, J = 16.9 Hz, 1H), 4.53 (d, J = 16.9 Hz, 1H), 3.67 (d, J = 12.8 Hz, 1H), 3.45 (d, J = 12.8 Hz, 1H), 2.54 (s, 3H). |
| #31 | 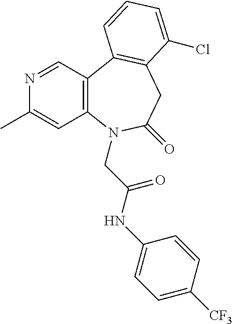 [SMILES: Cc1cc2c(cn1)-c1cccc(Cl)c1CC(=O)N2CC(=O)Nc1ccc(C(F)(F)F)cc1] | 2-(8-chloro-3-meth- yl-6-oxo-7H-pyrido [4,3-d][3]benzaze- pin-5-yl)-N-[4- (trifluoromethyl)- phenyl]acetamide | Starting from Intermediate N86. LC-MS (Method B2) m/z [M + H]⁺: 460.02; rt: 4.50 min; purity: 92%. LC-MS (Method A2) m/z [M + H]⁺: 459.99; rt: 3.69 min; purity: 90%. ¹H NMR (400 MHz, DMSO-d₆) δ 10.62 (s, 1H), 8.68 (s, 1H), 7.80-7.78 (m, 2H), 7.70-7.64 (m, 4H), 7.49 (t, J = 7.9 Hz, 1H), 7.34 (s, 1H), 4.64 (d, J = 16.9 Hz, 1H), 4.55 (d, J = 16.9 Hz, 1H), 4.13 (d, J = 12.8 Hz, 1H), 3.40 (d, J = 12.8 Hz, 1H), 2.55 (s, 3H) |
| #32 | 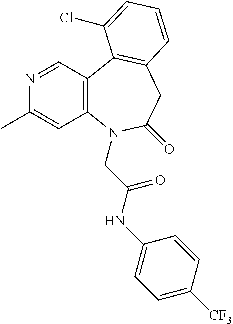 [SMILES: Cc1cc2c(cn1)-c1c(Cl)cccc1CC(=O)N2CC(=O)Nc1ccc(C(F)(F)F)cc1] | 2-(11-chloro-3- methyl-6-oxo-7H- pyrido[4,3-d][3] benzazepin-5-yl)- N-[4-(trifluoro- methyl)phenyl] acetamide | Starting from Intermediate N87. LC-MS (Method B3) m/z [M + H]⁺: 460.10; rt: 4.58 min; purity: 97%. |
| #33 | 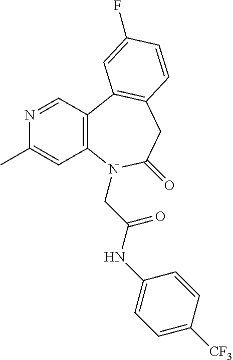 [SMILES: Cc1cc2c(cn1)-c1cc(F)ccc1CC(=O)N2CC(=O)Nc1ccc(C(F)(F)F)cc1] | 2-(10-fluoro-3- methyl-6-oxo-7H- pyrido[4,3-d][3] benzazepin-5-yl)- N-[4-(trifluoro- methyl)phenyl] acetamide | Starting from Intermediate N88. LC-MS (Method B2) m/z [M + H]⁺: 444.0; rt: 4.37 min; purity: 99%. LC-MS (Method A2) m/z [M + H]⁺: 444.0; rt: 3.73 min; purity: 98%. ¹H NMR (400 MHz, DMSO-d₆) δ 10.62 (bs, 1H), 8.70 (s, 1H), 7.79 (d, J = 8.4 Hz, 2H), 7.67 (d, J = 8.4 Hz, 2H), 7.61 (dd, J = 9.9, 2.7 Hz, 1H), 7.51 (dd, J = 8.5, 5.7 Hz, 1H), 7.37-7.26 (m, 2H), 4.62 (d, J = 16.8 Hz, 1H), 4.49 (d, J = 16.8 Hz, 1H), 3.66 (d, J = 12.9 Hz, 1H), 3.41 (d, J = 12.9 Hz, 1H), 2.54 (s, 3H) |
| #34 | 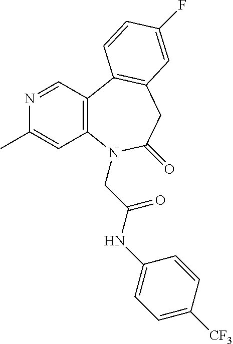 [SMILES: Cc1cc2c(cn1)-c1ccc(F)cc1CC(=O)N2CC(=O)Nc1ccc(C(F)(F)F)cc1] | 2-(9-fluoro-3- methyl-6-oxo-7H- pyrido[4,3-d][3] benzazepin-5-yl)- N-[4-(trifluoro- methyl)phenyl] acetamide | Starting from Intermediate N89. LC-MS (Method B2) m/z [M + H]⁺: 444.0; rt: 4.36 min; purity: 95%. LC-MS (Method A2) m/z [M + H]⁺: 444.0; rt: 3.71 min; purity: 90%. ¹H NMR (400 MHz, DMSO-d₆) δ 10.61 (s, 1H), 8.67 (s, 1H), 7.87-7.60 (m, 5H), 7.40-7.33 (m, 3H), 4.62 (d, J = 16.8 Hz, 1H), 4.50 (d, J = 16.8 Hz, 1H), 3.66 (d, J = 12.5 Hz, 1H), 3.48 (d, J = 12.5 Hz, 1H), 2.66 (s, 3H). |
| #35 | 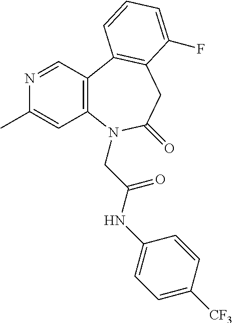 [SMILES: Cc1cc2c(cn1)-c1cccc(F)c1CC(=O)N2CC(=O)Nc1ccc(C(F)(F)F)cc1] | 2-(8-fluoro-3- methyl-6-oxo-7H- pyrido[4,3-d][3] benzazepin-5-yl)- N-[4-(trifluoro- methyl)phenyl] acetamide | Starting from Intermediate N90. LC-MS (Method B2) m/z [M + H]⁺: 444.0; rt: 4.41 min; purity: 97%. LC-MS (Method A2) m/z [M + H]⁺: 444.0; rt: 3.76 min; purity: 97%. ¹H NMR (400 MHz, DMSO-d₆) δ 10.65 (s, 1H), 8.70 (s, 1H), 7.78 (d, J = 8.7 Hz, 2H), 7.67 (d, J = 8.7 Hz, 2H), 7.58 (d, J = 7.8 Hz, 1H), 7.52 (t, J = 7.2 Hz, 1H), 7.39 (t, J = 8.9 Hz, 1H), 7.34 (s, 1H), 4.63 (d, J = 16.8 Hz, 1H), 4.52 (d, J = 16.8 Hz, 1H), 3.89 (d, J = 13.2 Hz, 1H), 3.28 (d, J = 13.2 Hz, 1H), 2.54 (s, 3H) |

### Example #36: 2-(5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

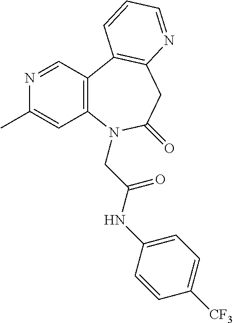 [SMILES: Cc1cc2c(cn1)-c1cccnc1CC(=O)N2CC(=O)Nc1ccc(C(F)(F)F)cc1]

A mixture of example #25 (195 mg, 0.41 mmol), potassium carbonate (148 mg, 1.06 mmol) and trimethylboroxine (270 μL, 1.9 mmol) in dioxane (4 mL) and water (0.5 mL) was degassed with argon for 1 min before the addition of PdCl₂dppf(II) (31 mg, 0.04 mmol). The reaction mixture was sealed under argon and heated at 90° C. for 8 h. After cooling to room temperature, the resulting suspension was filtered through a pad of Celite®, rinsed with EtOAc, and the filtrate was concentrated under vacuum. The crude dark oil was purified by preparative HPLC (acidic elution) to afford the title compound (10 mg, yield: 6%) as a white solid. LC-MS (Method B3) m/z [M+H]⁺: 427.14; rt: 4.13 min; purity>99%. LC-MS (Method A3) m/z [M+H]⁺: 427.14; rt: 3.72 min; purity>99%. ¹H NMR (400 MHz, DMSO-d₆) δ 10.64 (s, 1H), 8.73 (s, 1H), 8.63 (d, J=4.6 Hz, 1H), 8.20 (s, J=7.7 Hz, 1H), 7.80 (d, J=8.3 Hz, 2H), 7.69 (d, J=8.3 Hz, 2H), 7.55 (dd, 7.7, 4.6 Hz, 1H), 7.38 (s, 1H), 4.65 (d, J=16.8 Hz, 1H), 4.52 (d, J=16.8 Hz, 1H), 3.77 (s, 2H), 2.56 (s, 3H).

### Example #37: 2-(12-chloro-8-oxo-5,6,9,13-tetrazatricyclo[8.4.0.0^{2,6}]tetradeca-1(10),2,4,11,13-pentaen-9-yl)-N-[4-(trifluoromethyl)phenyl]acetamide

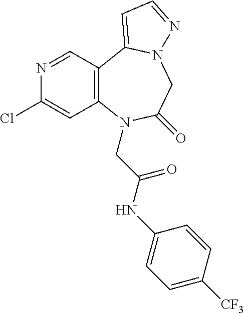 [SMILES: O=C(CN1C(=O)Cn2nccc2-c2cnc(Cl)cc21)Nc1ccc(C(F)(F)F)cc1]

The title product was prepared according to general procedure 1, starting from Intermediate N15 (25 mg, 0.106 mmol) and 2-chloro-N-(4-trifluoromethylphenyl)acetamide (CAS 2707-23-5). Purification by preparative HPLC (with basic elution) afforded the title product as a white solid (23 mg, yield: 50%). LC-MS (Method B2) m/z [M+H]⁺: 436.00; rt: 4.18 min; purity>99%. LC-MS (Method A2) m/z [M+H]⁺: 436.00; rt: 2.55 min; purity>99%. ¹H NMR (400 MHz, DMSO-d₆) δ 10.73 (s, 1H), 8.73 (s, 1H), 7.80 (d, J=8.3 Hz, 2H), 7.70 (d, J=8.3 Hz, 2H), 7.67-7.65 (m, 2H), 6.91 (s, 1H), 5.02 (s, 2H), 4.71 (s, 2H).

### Example #38: 2-(15-fluoro-13-methyl-9-oxo-3,10,14-triazatricyclo[9.4.0.0^{2,7}]pentadeca-1(11),2(7),3,5,12,14-hexaen-10-yl)-N-[4-(trifluoromethyl)phenyl]acetamide

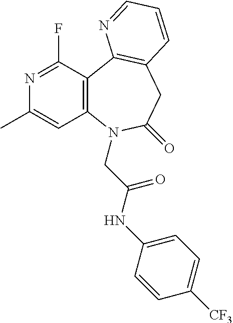 [SMILES: Cc1cc2c(c(F)n1)-c1ncccc1CC(=O)N2CC(=O)Nc1ccc(C(F)(F)F)cc1]

The title product was prepared according to general procedure 1, starting from Intermediate N16 (60 mg, 0.247 mmol) and 2-chloro-N-(4-trifluoromethylphenyl)acetamide (CAS 2707-23-5). It was obtained as a white solid (31 mg, yield: 28%). LC-MS (Method B3) m/z [M+H]⁺: 445.13; rt: 4.44 min; purity>99%. LC-MS (Method A3) m/z [M+H]⁺: 445.14; rt: 4.48 min; purity>99%. ¹H NMR (400 MHz, DMSO-d₆) δ 10.62 (s, 1H), 8.68 (d, J=4.6 Hz, 1H), 7.93 (d, J=7.7 Hz, 1H), 7.79 (d, J=8.4 Hz, 2H), 7.69 (d, J=8.4 Hz, 2H), 7.50 (dd, J=7.7, 4.6 Hz, 1H), 7.31 (s, 1H), 4.58 (s, 2H), 3.68 (s, 2H). 3H (CH₃) protons merged in solvent peak.

The following Examples #39-43 have been prepared according to general procedure 1 from commercially available 2-chloro-N-(4-trifluoromethylphenyl)acetamide (CAS 2707-23-5) and other specific intermediates referenced in the table below.

| Ex | Structure Example | Example Name | Protocolumn and characterization |
|---|---|---|---|
| #39 | 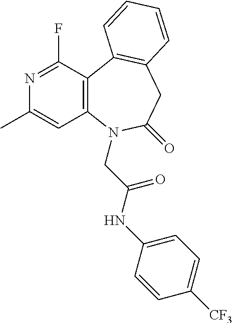 [SMILES: Cc1cc2c(c(F)n1)-c1ccccc1CC(=O)N2CC(=O)Nc1ccc(C(F)(F)F)cc1] | 2-(1-fluoro-3-methyl- 6-oxo-7H-pyrido[4,3- d][3]benzazepin-5-yl)- N-[4-(trifluorometh- yl)phenyl]acetamide | Starting Intermediate N113. LC-MS (Method B3) m/z [M + H]⁺: 444.13; rt: 5.06 min; purity >99%. LC-MS (Method A3) m/z [M + H]⁺: 444.13; rt: 5.27 min; purity: 99%. ¹H NMR (400 MHz, DMSO-d₆) δ 10.61 (s, 1H), 7.80 (d, J = 8.4 Hz, 2H), 7.70-7.68 (m, 3H), 7.49- 7.45 (m, 3H), 7.32 (s, 1H), 4.57 (d, J = 16.8 Hz, 1H), 4.52 (d, J = 16.8 Hz, 1H), 3.61 (d, J = 12.7 Hz, 1H), 3.57 (d, J = 12.7 Hz, 1H). 3H (CH3) protons merged in solvent peak. |
| #40 | 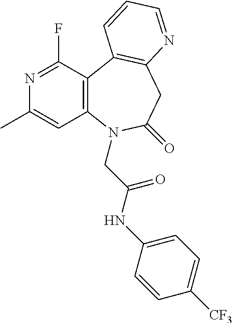 [SMILES: Cc1cc2c(c(F)n1)-c1cccnc1CC(=O)N2CC(=O)Nc1ccc(C(F)(F)F)cc1] | 2-(3-fluoro-5-methyl- 9-oxo-4,8,12-triaza- tricyclo[9.4.0.0^{2,7}] pentadeca-1(11),2(7), 3,5,12,14-hexaen-8- yl)-N-[4-(trifluoro- methyl)phenyl]acet- amide | Starting from Intermediate N114. LC- MS (Method B3) m/z [M + H]⁺: 445.13; rt: 4.22 min; purity: 97%. LC-MS (Method A3) m/z [M + H]⁺: 445.13; rt: 4.59 min; purity: 96%. ¹H NMR (400 MHz, DMSO-d₆) δ 10.63 (s, 1H), 8.62 (dd, J = 4.8, 1.7 Hz, 1H), 8.16 (ddd, J = 7.9, 4.8, 1.7 Hz, 1H), 7.79 (d, J = 8.5 Hz, 2H), 7.69 (d, J = 8.5 Hz, 2H), 7.53 (dd, J = 7.9, 4.8 Hz, 1H), 7.38 (s, 1H), 4.61 (s, 2H), 3.89 (d, J = 12.5 Hz, 1H), 3.74 (d, J = 12.5 Hz, 1H). 3H (CH3) protons merged in solvent peak. |
| #41 | 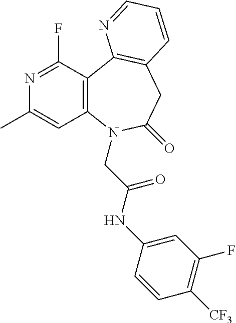 | N-(4-chloro-3-fluoro- phenyl)-2-(15-fluoro- 13-methyl-9-oxo- 3,10,14-triazatricyclo [9.4.0.0^{2,7}]pentadeca- 1(11),2(7),3,5,12,14- hexaen-10-yl)acetamide | Starting from Intermediate N115. LC- MS (Method B3) m/z [M + H]⁺: 429.0; rt: 3.99 min; purity >99%. LC-MS (Method A3) m/z [M + H]⁺: 429.0; rt: 4.55 min; purity >99%. ¹H NMR (400 MHz, DMSO-d₆) δ 10.58 (s, 1H), 8.68 (dd, J = 4.9, 1.8 Hz, 1H), 7.92 (d, J = 8.0 Hz, 1H), 7.75 (dd, J = 11.9, 2.5 Hz, 1H), 7.57-7.45 (m, 2H), 7.32 (dd, J = 8.9, 2.5 Hz, 1H), 7.29 (s, 1H), 4.55 (s, 2H), 3.67 (s, 2H), 2.49 (s, 3H). |
| #42 | 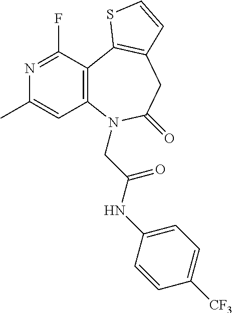 [SMILES: Cc1cc2c(c(F)n1)-c1sccc1CC(=O)N2CC(=O)Nc1ccc(C(F)(F)F)cc1] | 2-(14-fluoro-12-meth- yl-8-oxo-3-thia-9,13- diazatricyclo[8.4.0.0^{2,6}] tetradeca-1(10),2(6), 4,11,13-pentaen-9-yl)- N-[4-(trifluorometh- yl)phenyl]acetamide | Starting from Intermediate N116. LC- MS (Method B4) m/z [M + H]⁺: 448.0; rt: 4.32 min; purity: 98%. LC-MS (Method A4) m/z [M + H]⁺: 448.0; rt: 4.81 min; purity: 98%. ¹H NMR (400 MHz, DMSO-d₆) δ 10.63 (s, 1H), 7.85 (d, J = 5.1 Hz, 1H), 7.82 (d, J = 8.8 Hz, 2H), 7.70 (d, J = 8.8 Hz, 2H), 7.33 (s, 1H), 7.20 (d, J = 5.1 Hz, 1H), 4.64 (d, J = 15.9 Hz, 1H), 4.50 (d, J = 15.9 Hz, 1H), 3.85 (d, J = 13.4 Hz, 1H), 3.33 (d, J = 13.4 Hz, 1H), 2.47 (s, 3H) |
| #43 | 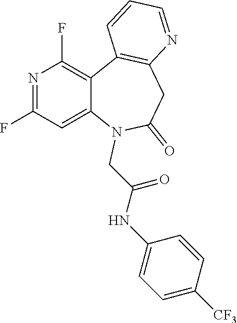 [SMILES: O=C(CN1C(=O)Cc2ncccc2-c2c1cc(F)nc2F)Nc1ccc(C(F)(F)F)cc1] | 2-(3,5-difluoro-9-oxo- 4,8,12-triazatricyclo [9.4.0.0^{2,7}]pentadeca- 1(11),2,4,6,12,14- hexaen-8-yl)-N-[4- (trifluoromethyl)phen- yl]acetamide | Starting from Intermediate N117. LC- MS (Method B5): m/z [M + H]⁺: 449.2; rt: 2.19 min; purity: 98%. ¹H NMR (400 MHz, DMSO-d₆) δ 10.69 (s, 1H), 8.64 (dd, J = 4.8, 1.6 Hz, 1H), 8.19 (ddd, J = 8.0, 4.7, 1.7 Hz, 1H), 7.77 (d, J = 8.5 Hz, 2H), 7.68 (d, J = 8.5 Hz, 2H), 7.54 (dd, J = 8.0, 4.8 Hz, 1H), 7.34 (s, 1H), 4.67 (s, 1H), 4.66 (s, 1H), 4.02 (d, J = 12.6 Hz, 1H), 3.75 (d, J = 12.6 Hz, 1H). |

### Example #44: 2-(9-chloro-3-methyl-6-oxo-7H-pyrido[4,3-d][3]benzazepin-5-yl)-N-[4-(trifluoromethyl)phenyl]acetamide

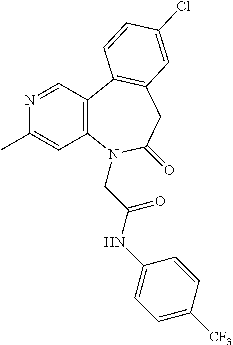 [SMILES: Cc1cc2c(cn1)-c1ccc(Cl)cc1CC(=O)N2CC(=O)Nc1ccc(C(F)(F)F)cc1]

The title product was prepared according to general procedure 1, starting from Intermediate N17 (92 mg, 0.36 mmol) and 2-chloro-N-(4-trifluoromethylphenyl)acetamide (CAS 2707-23-5). Purification by trituration in DCM afforded the title product as a white solid (36 mg, yield: 22%). LC-MS (Method B2) m/z [M+H]⁺: 459.94; rt: 4.55 min; purity: 96%. LC-MS (Method A2) m/z [M+H]⁺: 459.98; rt: 3.90 min; purity: 93%. ¹H NMR (400 MHz, DMSO-d₆) δ 10.63 (s, 1H), 8.68 (s, 1H), 7.80 (d, J=8.3 Hz, 2H), 7.75 (d, J=8.3 Hz, 1H), 7.69 (d, J=8.3 Hz, 2H), 7.64 (s, 1H), 7.56 (d, J=8.3 Hz, 1H), 7.34 (s, 1H), 4.63 (d, J=16.8 Hz, 1H), 4.52 (d, J=16.8 Hz, 1H), 3.69 (d, J=12.8 Hz, 1H), 3.50 (d, J=12.8 Hz, 1H), 2.54 (s, 3H).

### Example #45: 2-(1-methyl-6-oxo-7H-pyrido[3,4-d][3]benzazepin-5-yl)-N-[4-(trifluoromethyl)phenyl]acetamide

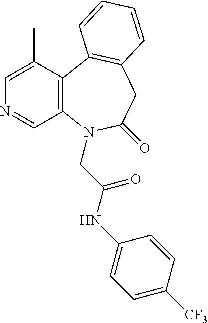 [SMILES: Cc1cncc2c1-c1ccccc1CC(=O)N2CC(=O)Nc1ccc(C(F)(F)F)cc1]

The title product was prepared according to general procedure 1 starting from Intermediate N91 and 2-chloro-N-(4-trifluoromethylphenyl)acetamide (CAS 2707-23-5). LC-MS (Method B2) m/z [M+H]⁺: 426.01; rt: 3.93 min; purity: 97%. LC-MS (Method A2) m/z [M+H]⁺: 426.02; rt: 4.29 min; purity: 98%.

### Example #46: 2-[3-(4-methyl-3-oxo-piperazin-1-yl)-6-oxo-7H-pyrido[4,3-d][3]benzazepin-5-yl]-N-[4-(trifluoromethyl)phenyl]acetamide

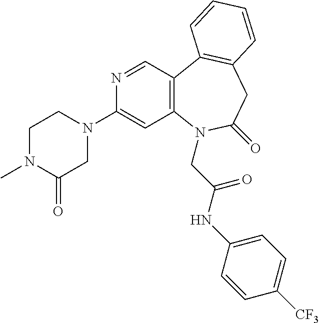 [SMILES: CN1CCN(c2cc3c(cn2)-c2ccccc2CC(=O)N3CC(=O)Nc2ccc(C(F)(F)F)cc2)CC1=O]

To a solution of example #13 (20 mg, 0.04 mmol) in 1-methyl-2-pyrrolidinone (0.5 mL) were added N,N-diisopropylethylamine (30 μL, 0.18 mmol) and 1-methylpiperazin-2-one (90 mg, 0.69 mmol) and the reaction mixture was stirred at 150° C. for 40 h. After cooling to room temperature, the crude solution was purified by preparative HPLC (with acidic elution) to afford the title compound (7 mg, yield: 28%) as a brown solid. LC-MS (Method B4) m/z [M+H]⁺: 524.19; rt: 3.91 min; purity: 96%. LC-MS (Method A4) m/z [M+H]⁺: 524.19; rt: 4.27 min; purity: 98%. ¹H NMR (400 MHz, DMSO-d₆) δ 10.55 (s, 1H), 8.39 (s, 1H), 7.80 (d, J=8.5 Hz, 2H), 7.68 (d, J=8.5 Hz, 2H), 7.63 (d, J=7.4 Hz, 1H), 7.46-7.40 (m, 3H), 6.77 (s, 1H), 4.61 (s, 2H), 4.19 (d, J=17.4 Hz, 1H), 4.03 (d, J=17.4 Hz, 1H), 4.00-3.93 (m, 1H), 3.83-3.76 (m, 1H), 3.57 (d, J=12.8 Hz, 1H), 3.49-3.42 (m, 3H), 2.90 (s, 3H).

### Example #47: 2-[3-(3,3-difluoroazetidin-1-v)-6-oxo-7H-pyrido[4,3-d][3]benzazepin-5-yl]-N-[4-(trifluoromethyl)phenyl]acetamide

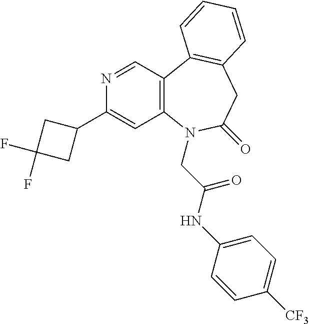 [SMILES: O=C(CN1C(=O)Cc2ccccc2-c2cnc(C3CC(F)(F)C3)cc21)Nc1ccc(C(F)(F)F)cc1]

The title product was prepared according to a similar procedure as the one depicted for example #46, using 3,3-difluoroazetidine instead of 1-methylpiperazin-2-one. LC-MS (Method B3) m/z [M+H]⁺: 503.15; rt: 5.26 min; purity>99%. LC-MS (Method A3) m/z [M+H]⁺: 503.15; rt: 5.35 min; purity: 99%. ¹H NMR (400 MHz, DMSO-d₆) δ 10.58 (s, 1H), 8.40 (s, 1H), 7.78 (d, J=8.8 Hz, 2H), 7.68 (d, J=8.8 Hz, 2H), 7.64 (d, J=7.5 Hz, 1H), 7.48-7.40 (m, 3H), 6.56 (s, 1H), 4.62-4.38 (m, 6H), 3.58 (d, J=12.8 Hz, 1H), 3.48 (d, J=12.8 Hz, 1H).

### Example #48: 2-(12-methyl-8-oxo-5,6,9,13-tetrazatricyclo[8.4.0.0^{2,6}]tetradeca-1(10),2,4,11,13-pentaen-9-yl)-N-[4-(trifluoromethyl)phenyl]acetamide

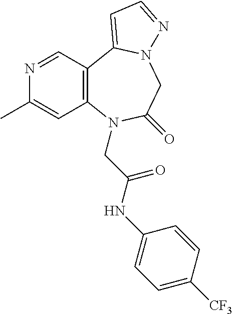 [SMILES: Cc1cc2c(cn1)-c1ccnn1CC(=O)N2CC(=O)Nc1ccc(C(F)(F)F)cc1]

The title product was prepared according to general procedure 1, starting from Intermediate N18 (23 mg, 0.107 mmol) and 2-chloro-N-(4-trifluoromethylphenyl)acetamide (CAS 2707-23-5). Purification by preparative HPLC (with basic elution) afforded the title product as a white solid (25 mg, yield: 56%). LC-MS (Method B2) m/z [M+H]⁺: 416.09; rt: 3.86 min; purity>99%. LC-MS (Method A2) m/z [M+H]⁺: 416.08; rt: 3.58 min; purity>99%. ¹H NMR (400 MHz, DMSO-d₆) δ 10.69 (s, 1H), 8.73 (s, 1H), 7.80 (d, J=8.3 Hz, 2H), 7.70 (d, J=8.3 Hz, 2H), 7.63 (s, 1H), 7.38 (s, 1H), 6.84 (s, 1H), 4.95 (s, 2H), 4.64 (s, 2H), 2.52 (s, 3H).

### Example #49: 2-(1-chloro-3-methyl-6-oxo-7H-pyrido[4,3-d][3]benzazepin-5-yl)-N-[4-(trifluoromethyl)phenyl]acetamide

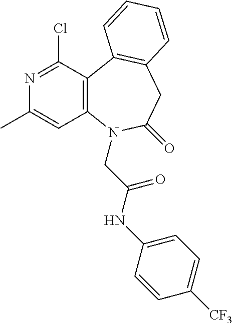 [SMILES: Cc1cc2c(c(Cl)n1)-c1ccccc1CC(=O)N2CC(=O)Nc1ccc(C(F)(F)F)cc1]

The title product was prepared according to general procedure 1, starting from Intermediate N19 (23 mg, 0.107 mmol) and 2-chloro-N-(4-trifluoromethylphenyl)acetamide (CAS 2707-23-5) as a white solid (85 mg, yield: 80%). LC-MS (Method B3) m/z [M+H]⁺: 460.06; rt: 4.78 min; purity: 97%. LC-MS (Method A3) m/z [M+H]⁺: 460.06; rt: 4.83 min; purity: 96%. ¹H NMR (400 MHz, DMSO-d₆) δ 10.59 (s, 1H), 7.81-7.78 (m, 3H), 7.69 (d, J=8.5 Hz, 2H), 7.49-7.44 (m, 3H), 7.39 (s, 1H), 4.51 (s, 2H), 3.56 (s, 2H). 3H (CH₃) protons merged in solvent peak.

The following Examples #50-54 have been prepared according to general procedure 1 starting respectively from chloro-N-(3-fluoro-4-chloro-phenyl)acetamide (CAS 895641-02-8) and chloro-N-(4-trifluoromethylphenyl)acetamide (CAS 2707-23-5), and other specific intermediates referenced in the table below.

| Ex | Structure Example | Example Name | Protocolumn and characterization |
|---|---|---|---|
| #50 | 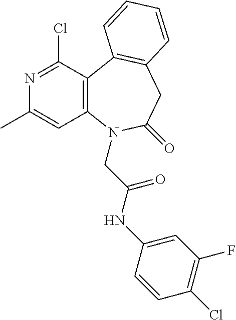 [SMILES: Cc1cc2c(c(Cl)n1)-c1ccccc1CC(=O)N2CC(=O)Nc1ccc(Cl)c(F)c1] | N-(4-chloro-3-fluoro- phenyl)-2-(1-chloro- 3-methyl-6-oxo-7H- pyrido[4,3-d][3]benz- azepin-5-yl)acetamide | Starting from Intermediate N19. LC- MS (Method B2) m/z [M + H]⁺: 443.98; rt: 4.71 min; purity >99%. LC-MS (Method A2′) m/z [M + H]⁺: 443.93; rt: 4.78 min; purity >99%. ¹H NMR (400 MHz, DMSO-d₆) δ 10.63 (s, 1H), 7.80-7.74 (m, 2H), 7.53 (t, J = 8.7 Hz, 1H), 7.48-7.41 (m, 3H), 7.38 (s, 1H), 7.33-7.30 (m, 1H), 4.48 (s, 2H), 3.51 (s, 2H), 2.51 (s, 3H). |
| #51 | 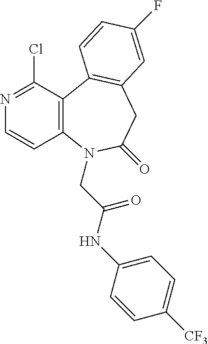 [SMILES: O=C(CN1C(=O)Cc2cc(F)ccc2-c2c1ccnc2Cl)Nc1ccc(C(F)(F)F)cc1] | 2-(1-chloro-9-fluoro- 6-oxo-7H-pyrido[4,3- d][3]benzazepin-5-yl)- N-[4-(trifluoromethyl)- phenyl]acetamide | Starting from Intermediate N92. LC- MS (Method B2) m/z [M − H]⁻: 462.1; rt: 4.61 min; purity: 97%. LC- MS (Method A2) m/z [M + H]⁺: 463.9; rt: 4.75 min; purity: 94%. ¹H NMR (400 MHz, DMSO-d₆) δ 10.60 (s, 1H), 8.43 (d, J = 5.5 Hz, 1H), 7.88 (dd, J = 8.8, 5.5 Hz, 1H), 7.77 (d, J = 8.8 Hz, 2H), 7.68 (d, J = 8.9 Hz, 2H), 7.52 (d, J = 5.6 Hz, 1H), 7.41 (dd, J = 9.3, 2.8 Hz, 1H), 7.30 (td, J = 8.7, 2.8 Hz, 1H), 4.56 (s, 2H), 3.61 (s, 2H). |
| #52 | 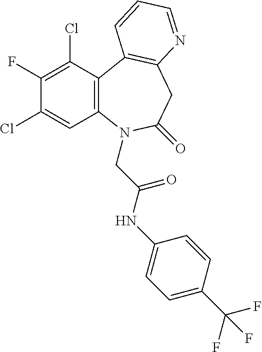 [SMILES: O=C(CN1C(=O)Cc2ncccc2-c2c1cc(Cl)c(F)c2Cl)Nc1ccc(C(F)(F)F)cc1] | 2-(9,11-dichloro-10- fluoro-6-oxo-5H- pyrido[2,3-d][1]benz- azepin-7-yl)-N-[4- (trifluoromethyl)phen- yl]acetamide | Starting from Intermediate N93. LC- MS (Method B4) m/z [M + H]⁺: 498.0; rt: 4.58 min; purity: 98%. LC- MS (Method A4) m/z [M + H]⁺: 498.0; rt: 5.07 min; purity: 97%. |
| #53 | 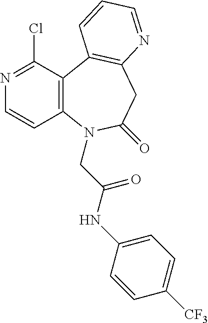 [SMILES: O=C(CN1C(=O)Cc2ncccc2-c2c1ccnc2Cl)Nc1ccc(C(F)(F)F)cc1] | 2-(3-chloro-9-oxo- 4,8,12-triazatricyclo [9.4.0.0^{2,7}]pentadeca- 1(11),2(7),3,5,12,14- hexaen-8-yl)-N-[4- (trifluoromethyl)phen- yl]acetamide | Starting from Intermediate N94. LC- MS (Method B4) m/z [M + H]⁺: 447.0; rt: 3.76 min; purity: 99%. LC- MS (Method A4) m/z [M + H]⁺: 447.0; rt: 4.18 min; purity: 97%. ¹H NMR (400 MHz, DMSO-d₆) δ 10.61 (s, 1H), 8.63 (d, J = 4.8 Hz, 1H), 8.47 (d, J = 5.6 Hz, 1H), 8.29 (d, J = 8.0 Hz, 1H), 7.76 (d, J = 8.8 Hz, 2H), 7.68 (d, J = 8.8 Hz, 2H), 7.57 (d, J = 5.6 Hz, 1H), 7.52 (dd, J = 8.0, 4.8 Hz, 1H), 4.60 (d, J = 17.1 Hz, 1H), 4.57 (d, J = 17.1 Hz, 1H), 3.92 (d, J = 12.7 Hz, 1H), 3.70 (d, J = 12.7 Hz, 1H). |
| #54 | 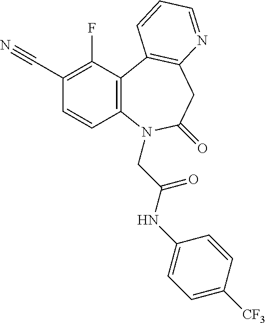 [SMILES: N#Cc1ccc2c(c1F)-c1cccnc1CC(=O)N2CC(=O)Nc1ccc(C(F)(F)F)cc1] | 2-(10-cyano-11- fluoro-6-oxo-5H- pyrido[2,3-d][1] benzazepin-7-yl)-N- [4-(trifluoromethyl)- phenyl]acetamide | Starting from Intermediate N95. LC- MS (Method B2) m/z [M + H]⁺: 455.1; rt: 4.27 min; purity: 98%. LC- MS (Method A2) m/z [M + H]⁺: 455.0; rt: 4.25 min; purity: 97%. ¹H NMR (400 MHz, DMSO-d₆) δ 10.62 (s, 1H), 8.66 (dd, J = 4.8, 1.8 Hz, 1H), 8.25-8.17 (m, 1H), 8.12-8.03 (m, 1H), 7.76 (d, J = 8.9 Hz, 2H), 7.68 (d, J = 8.9 Hz, 2H), 7.60 (d, J = 8.7 Hz, 1H), 7.55 (dd, J = 7.9, 4.8 Hz, 1H), 4.60 (s, 2H), 3.90 (d, J = 12.5 Hz, 1H), 3.72 (d, J = 12.5 Hz, 1H). |

### Example #55: 2-(1-hydroxy-3-methyl-6-oxo-7H-pyrido[4,3-d][3]benzazepin-5-yl)-N-[4-(trifluoromethyl)phenyl]acetamide

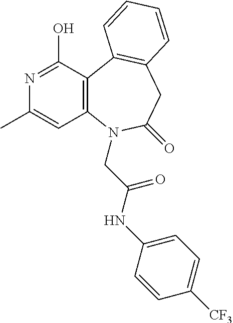 [SMILES: Cc1cc2c(c(O)n1)-c1ccccc1CC(=O)N2CC(=O)Nc1ccc(C(F)(F)F)cc1]

A suspension of example #39 (20 mg, 0.04 mmol) in acetic acid (0.5 mL) and water (50 μL) was heated at 140° C. for 1.5 h using microwave irradiation. The reaction mixture was concentrated to dryness and the crude residue was partitioned between EtOAc and saturated aqueous NaHCO₃. The organic phase was dried with brine and concentrated under vacuum. The resulting solid was purified by preparative HPLC (with basic elution) to afford the title compound (8 mg, yield: 40%) as a white solid. LC-MS (Method B3) m/z [M+H]⁺: 442.14; rt: 4.22 min; purity: 99%. LC-MS (Method A3) m/z [M+H]⁺: 442.14; rt: 4.22 min; purity>99%. ¹H NMR (400 MHz, DMSO-d₆) δ 11.90 (bs, 1H), 10.67 (s, 1H), 7.90-7.88 (m, 1H), 7.79 (d, J=8.7 Hz, 2H), 7.68 (d, J=8.7 Hz, 2H), 7.34-7.30 (m, 3H), 6.17 (s, 1H), 4.39 (d, J=16.7 Hz, 1H), 4.33 (d, J=16.7 Hz, 1H), 3.54 (d, J=12.3 Hz, 1H), 3.38 (d, J=12.3 Hz, 1H), 2.17 (s, 3H).

### Example #56: 2-[1-(difluoromethyl)-3-methyl-6-oxo-7H-pyrido[4,3-d][3]benzazepin-5-yl]-N-[4-(trifluoromethyl)phenyl]acetamide

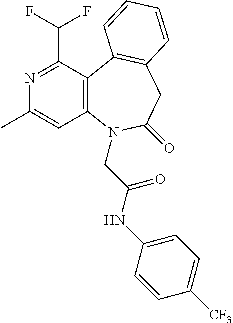 [SMILES: Cc1cc2c(c(C(F)F)n1)-c1ccccc1CC(=O)N2CC(=O)Nc1ccc(C(F)(F)F)cc1]

A suspension of example #14 (10 mg, 0.02 mmol) and (bis(difluoroacetoxy)iodo)benzene (30 mg, 0.08 mmol) in DCM (0.5 mL) was degassed under argon and sonicated. The resulting mixture was irradiated in a Pennoc reactor (365 nm; Fan=4000; Stirr=400; LED=100%) for 2 h. The solvent was evaporated to dryness and the crude solid was purified by preparative HPLC (with basic elution) to afford the title compound (470 μg, yield: 4%) as a white solid. LC-MS (Method B3) m/z [M+H]⁺: 476.14; rt: 5.02 min; purity: 99%. LC-MS (Method A3) m/z [M+H]⁺: 476.14; rt: 5.15 min; purity: 97%. ¹H NMR (400 MHz, DMSO-d₆) δ 10.59 (s, 1H), 7.79 (d, J=8.9 Hz, 2H), 7.69 (d, J=8.9 Hz, 2H), 7.58-7.47 (m, 4H), 7.44 (d, J=7.2 Hz, 1H), 6.75 (t, J=53.6 Hz, 1H), 4.55 (s, 2H), 3.60 (d, J=12.8 Hz, 1H), 3.56 (d, J=12.8 Hz, 1H), 2.60 (s, 3H).

### Example #57: 2-(3,5-dimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

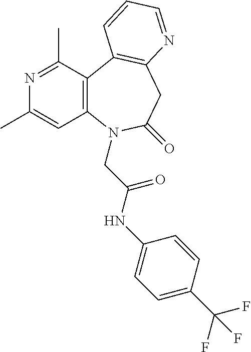 [SMILES: Cc1cc2c(c(C)n1)-c1cccnc1CC(=O)N2CC(=O)Nc1ccc(C(F)(F)F)cc1]

The title product was prepared according to general procedure 1, starting from Intermediate N20 (114 mg, 0.476 mmol) and 2-chloro-N-(4-trifluoromethylphenyl)acetamide (CAS 2707-23-5). Purification by preparative HPLC (with basic elution) afforded the title product as a white solid (24 mg, yield: 9%). LC-MS (Method B2) m/z [M+H]⁺: 441.15; rt: 4.31 min; purity>99%. LC-MS (Method A2) m/z [M+H]⁺: 441.12; rt: 3.25 min; purity>99%. ¹H NMR (400 MHz, DMSO-d₆) δ 10.59 (s, 1H), 8.57 (d, J=4.8 Hz, 1H), 8.11 (d, J=7.8 Hz, 1H), 7.77 (d, J=8.5 Hz, 2H), 7.67 (d, J=8.5 Hz, 2H), 7.47 (dd, J=7.8, 4.8 Hz, 1H), 7.23 (s, 1H), 4.52 (s, 2H), 3.77 (d, J=12.3 Hz, 1H), 3.65 (d, J=12.3 Hz, 1H). 6H (2 CH₃) protons merged in solvent peak.

### Example #58: N-(4-cyclopropylphenyl)-2-(3,5-dimethyl-9-oxo-4,8,12-triazatricyclo[9.4.0.0^{2,7}]pentadeca-1(11),2(7),3,5,12,14-hexaen-8-yl)acetamide

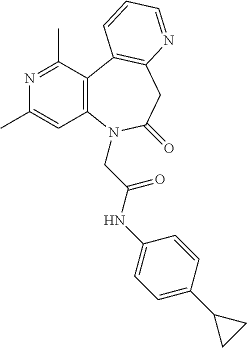 [SMILES: Cc1cc2c(c(C)n1)-c1cccnc1CC(=O)N2CC(=O)Nc1ccc(C2CC2)cc1]

The title product was prepared according to general procedure 1 starting from intermediate N20 and 2-chloro-N-(4-cyclopropylphenyl)acetamide (Intermediate N129). LC-MS (Method B2) m/z [M+H]⁺: 413.1; rt: 3.78 min; purity>99%. LC-MS (Method A2) m/z [M+H]⁺: 413.1; rt: 3.09 min; purity>99%. ¹H NMR (400 MHz, DMSO-d₆) δ 10.06 (s, 1H), 8.56 (dd, J=4.8, 1.8 Hz, 1H), 8.10 (dd, J=7.9, 1.8 Hz, 1H), 7.46 (dd, J=7.9, 4.8 Hz, 1H), 7.42 (d, J=8.7 Hz, 2H), 7.22 (s, 1H), 7.00 (d, J=8.7 Hz, 2H), 4.45 (s, 2H), 3.75 (d, J=12.3 Hz, 1H), 3.64 (d, J=12.3 Hz, 1H), 2.51 (s, 3H), 2.49 (s, 3H), 1.91-1.80 (m, 1H), 0.94-0.83 (m, 2H), 0.64-0.56 (m, 2H).

### Example #59: [6-oxo-5-[2-oxo-2-[4-(trifluoromethyl)anilino]ethyl]-7H-pyrido[4,3-d][3]benzazepin-3-yl]methyl acetate

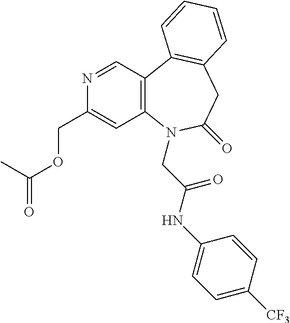 [SMILES: CC(=O)OCc1cc2c(cn1)-c1ccccc1CC(=O)N2CC(=O)Nc1ccc(C(F)(F)F)cc1]

A suspension of example #14 (50 mg, 0.10 mmol) in acetic anhydride (1.5 mL) was heated at 100° C. for 1 h using microwaves with a power output ranging from 0 to 100 W. The reaction mixture was evaporated to dryness. Purification by column chromatography on silica gel (using a gradient of 20% to 100% EtOAc in heptane as eluent) afforded the title compound as a light brown solid (24 mg, yield: 39%). A fraction of the desired compound (8 mg) was further purified by preparative HPLC (with acidic elution) to afford the title compound (4 mg, yield: 8%) as a white solid. LC-MS (Method B2) m/z [M+H]⁺: 484.16; rt: 4.30 min; purity>99%. LC-MS (Method A2) m/z [M+H]⁺: 484.16; rt: 4.40 min; purity>99%. ¹H NMR (400 MHz, DMSO-d₆) δ 10.65 (s, 1H), 8.80 (s, 1H), 7.80 (d, J=8.5 Hz, 2H), 7.78-7.75 (m, 1H), 7.70 (d, J=8.5 Hz, 2H), 7.53-7.47 (m, 3H), 7.45 (s, 1H), 5.23 (d, J=13.6 Hz, 1H), 5.17 (d, J=13.6 Hz, 1H), 4.68 (d, J=17.1 Hz, 1H), 4.47 (d, J=17.1 Hz, 1H), 3.67 (d, J=12.8 Hz, 1H), 3.50 (d, J=12.8 Hz, 1H), 2.06 (s, 3H).

### Example #60: 2-[3-(hydroxymethyl)-6-oxo-7H-pyrido[4,3-d][3]benzazepin-5-yl]-N-[4-(trifluoromethyl)phenyl]acetamide

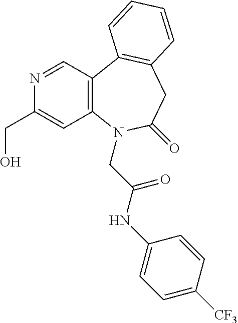 [SMILES: O=C(CN1C(=O)Cc2ccccc2-c2cnc(CO)cc21)Nc1ccc(C(F)(F)F)cc1]

A suspension of example #59 (49 mg, 0.08 mmol) in a 4 M solution of HCl in 1,4-dioxane (0.8 mL, 1.6 mmol) was stirred at 60° C. for 50 h. The reaction mixture was evaporated to dryness to give a dark brown solid. Purification by preparative HPLC (with basic elution) afforded the title compound (7 mg, yield: 19%) as an off-white solid. LC-MS (Method B3) m/z [M+H]⁺: 442.14; rt: 4.32 min; purity: 95%. LC-MS (Method A3) m/z [M+H]⁺: 442.14; rt: 4.03 min; purity: 94%. ¹H NMR (400 MHz, DMSO-d₆) δ 10.66 (s, 1H), 8.73 (s, 1H), 7.81 (d, J=8.7 Hz, 2H), 7.78-7.73 (m, 1H), 7.70 (d, J=8.7 Hz, 2H), 7.52-7.48 (m, 4H), 5.53 (t, J=5.8 Hz, 1H), 4.68-4.62 (m, 3H), 4.42 (d, J=17.2 Hz, 1H), 3.66 (d, J=12.7 Hz, 1H), 3.48 (d, J=12.7 Hz, 1H).

### Example #61: 2-(1-cyano-3-methyl-6-oxo-7H-pyrido[4,3-d][3]benzazepin-5-yl)-N-[4-(trifluoromethyl)phenyl]acetamide

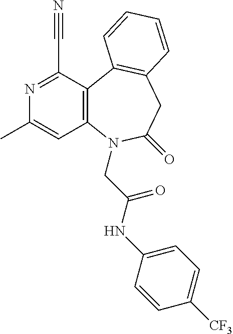 [SMILES: Cc1cc2c(c(C#N)n1)-c1ccccc1CC(=O)N2CC(=O)Nc1ccc(C(F)(F)F)cc1]

To a solution of example #39 (20 mg, 0.045 mmol) in dry DMA (0.5 mL) was added potassium cyanide (14 mg, 0.20 mmol) and the resulting mixture was stirred at 120° C. for 54 h. After cooling to room temperature, saturated aqueous NaHCO₃ was added and the reaction mixture was extracted 3 times with EtOAc. The combined organic layers were washed with water, dried with brine and over Na₂SO₄, filtered off and concentrated under vacuum. The crude solid was purified by preparative HPLC to afford the title compound (2 mg, yield: 11%) as a white solid. LC-MS (Method B3) m/z [M+H]: 451.14; rt: 4.82 min; purity>99%. LC-MS ((Method A3) m/z [M+H]⁺: 451.04; rt: 5.08 min; purity: 99%. ¹H NMR (400 MHz, DMSO-d₆) δ 10.68 (s, 1H), 7.90 (d, J=8.3 Hz, 1H), 7.79 (d, J=8.6 Hz, 2H), 7.69 (d, J=8.6 Hz, 2H), 7.66 (s, 1H), 7.60-7.52 (m, 3H), 4.60 (d, J=16.8 Hz, 1H), 4.54 (d, J=16.8 Hz, 1H), 3.67 (d, J=12.8 Hz, 1H), 3.62 (d, J=12.8 Hz, 1H), 2.59 (s, 3H).

### Example #62: 2-(9-chloro-1,3-dimethyl-6-oxo-7H-pyrido[4,3-d][3]benzazepin-5-yl)-N-[4-(trifluoromethyl)phenyl]acetamide

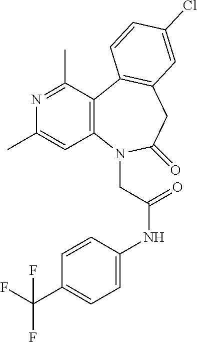 [SMILES: Cc1cc2c(c(C)n1)-c1ccc(Cl)cc1CC(=O)N2CC(=O)Nc1ccc(C(F)(F)F)cc1]

The title product was prepared according to general procedure 1, starting from Intermediate N21 (107 mg, 0.392 mmol) and 2-chloro-N-(4-trifluoromethylphenyl)acetamide (CAS 2707-23-5). Purification by trituration in DCM afforded the title product as a beige solid (146 mg, yield: 63%). LC-MS (Method B2) m/z [M+H]⁺: 473.9; rt: 4.67 min; purity: 97%. LC-MS (Method A2) m/z [M+H]⁺: 474.0; rt: 3.80 min; purity: 96%. ¹H NMR (400 MHz, CDCl₃) δ 8.35 (bs, 1H), 7.53 (d, J=8.8 Hz, 2H), 7.48-7.44 (m, 3H), 7.39 (d, J=8.3 Hz, 1H), 7.28 (s, 1H), 7.26 (s, 1H), 4.61 (d, J=15.6 Hz, 1H), 4.23 (d, J=15.6 Hz, 1H), 3.59 (d, J=12.8 Hz, 1H), 3.51 (d, J=12.8 Hz, 1H), 2.61 (s, 6H).

The following Examples #63-69 have been prepared according to general procedure 1 starting from commercially available 2-chloro-N-(4-trifluoromethylphenyl)acetamide (CAS 2707-23-5) and other specific intermediates referenced in the table below.

| Ex | Structure Example | Example Name | Protocolumn and characterization |
|---|---|---|---|
| #63 | 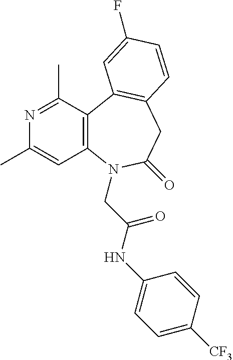 [SMILES: Cc1cc2c(c(C)n1)-c1cc(F)ccc1CC(=O)N2CC(=O)Nc1ccc(C(F)(F)F)cc1] | 2-(10-fluoro-1,3-dimeth- yl-6-oxo-7H-pyrido[4,3- d][3]benzazepin-5-yl)- N-[4-(trifluoromethyl)- phenyl]acetamide | Starting from Intermediate N96. LC-MS (Method B4) m/z [M + H]⁺: 458.0; rt: 4.16 min; purity >99%. LC-MS (Method A4) m/z [M + H]⁺: 458.0; rt: 3.56 min; purity >99%. ¹H NMR (400 MHz, CDCl₃) δ 8.34 (bs, 1H), 7.56-7.45 (m, 4H), 7.40 (dd, J = 8.5, 5.5 Hz, 1H), 7.29 (s, 1H), 7.24 (dd, J = 9.4, 2.6 Hz, 1H), 7.16 (td, J = 8.5, 2.6 Hz, 1H), 4.62 (d, J = 15.4 Hz, 1H), 4.22 (d, J = 15.4 Hz, 1H), 3.61 (d, J = 12.9 Hz, 1H), 3.47 (d, J = 12.9 Hz, 1H), 2.64 (s, 3H), 2.62 (s, 3H) |
| #64 | 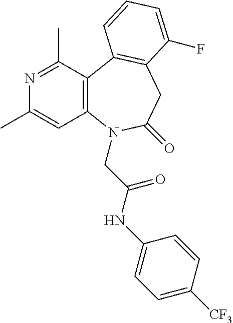 [SMILES: Cc1cc2c(c(C)n1)-c1cccc(F)c1CC(=O)N2CC(=O)Nc1ccc(C(F)(F)F)cc1] | 2-(8-fluoro-1,3-dimeth- yl-6-oxo-7H-pyrido [4,3-d][3]benzazepin-5- yl)-N-[4-(trifluorometh- yl)phenyl]acetamide | Starting from Intermediate N97. LC-MS (Method B4) m/z [M + H]⁺: 458.0; rt: 4.21 min; purity >99%. LC-MS (Method A4) m/z [M + H]⁺: 458.0; rt: 3.61 min; purity: 98%. |
| #65 | 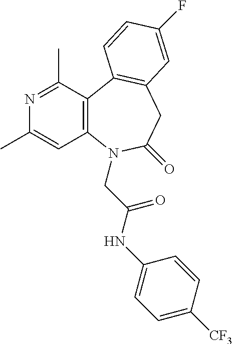 [SMILES: Cc1cc2c(c(C)n1)-c1ccc(F)cc1CC(=O)N2CC(=O)Nc1ccc(C(F)(F)F)cc1] | 2-(9-fluoro-1,3-dimeth- yl-6-oxo-7H-pyrido[4,3- d][3]benzazepin-5-yl)- N-[4-(trifluorometh- yl)phenyl]acetamide | Starting from Intermediate N98. LC-MS (Method B4) m/z [M + H]⁺: 458.0; rt: 4.17 min; purity >99%. LC-MS (Method A4) m/z [M + H]⁺: 458.0; rt: 3.55 min; purity: 97%. ¹H NMR (400 MHz, DMSO-d₆) δ 10.55 (s, 1H), 7.78 (d, J = 8.7 Hz, 2H), 7.72-7.64 (m, 3H), 7.37 (dd, J = 9.3, 2.8 Hz, 1H), 7.25 (td, J = 8.7, 2.8 Hz, 1H), 7.18 (s, 1H), 4.51 (d, J = 16.7 Hz, 1H), 4.46 (d, J = 16.7 Hz, 1H), 3.55 (d, J = 12.5 Hz, 1H), 3.46 (d, J = 12.5 Hz, 1H), 2.48 (s, 6H) |
| #66 | 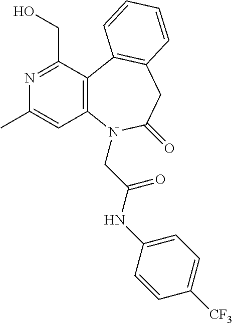 [SMILES: Cc1cc2c(c(CO)n1)-c1ccccc1CC(=O)N2CC(=O)Nc1ccc(C(F)(F)F)cc1] | 2-[1-(hydroxymethyl)- 3-methyl-6-oxo-7H- pyrido[4,3-d][3]benz- azepin-5-yl]-N-[4- (trifluoromethyl)phen- yl]acetamide | Starting from Intermediate N99; purity: 95%. LC-MS (Method A2) m/z [M + H]⁺: 456.4; rt: 3.50 min; purity: 95%. ¹H NMR (400 MHz, DMSO-d₆) δ 10.57 (s, 1H), 7.81-7.76 (m, 3H), 7.69 (d, J = 9.2 Hz, 2H), 7.48-7.41 (s, 3H), 7.29 (s, 1H), 5.25 (s, 1H), 4.75-4.31 (m, 4H), 3.53 (d, J = 12.4 Hz, 1H), 3.46 (d, J = 12.4 Hz, 1H), 2.55 (s, 3H) |
| #67 | 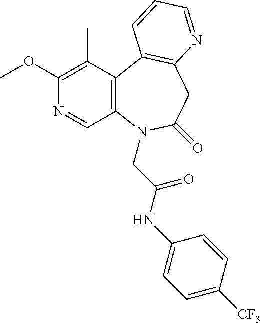 [SMILES: COc1ncc2c(c1C)-c1cccnc1CC(=O)N2CC(=O)Nc1ccc(C(F)(F)F)cc1] | 2-(4-methoxy-3-meth- yl-9-oxo-5,8,12-triaza- tricyclo[9.4.0.0^{2,7}] pentadeca-1(11),2(7), 3,5,12,14-hexaen-8-yl)- N-[4-(trifluoromethyl)- phenyl]acetamide | Starting from Intermediate N100. LC- MS (Method B2) m/z [M + H]⁺: 457.0; rt: 4.36 min; purity: 95%. LC-MS (Method A2) m/z [M + H]⁺: 457.0; rt: 4.22 min; purity: 96%. ¹H NMR (400 MHz, DMSO-d₆) δ 10.54 (s, 1H), 8.61 (d, J = 5.0 Hz, 1H), 8.32 (s, 1H), 8.06 (d, J = 7.2 Hz, 1H), 7.73 (d, J = 8.7 Hz, 2H), 7.65 (d, J = 8.7 Hz, 2H), 7.51 (dd, J = 7.2, 5.0 Hz, 1H), 4.60 (d, J = 16.6 Hz, 1H), 4.49 (d, J = 16.6 Hz, 1H), 3.95 (s, 3H), 3.80 (d, J = 12.3 Hz, 1H), 3.59 (d, J = 12.3 Hz, 1H), 2.18 (s, 3H). |
| #68 | 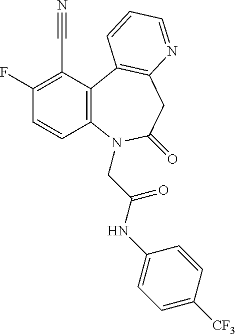 [SMILES: N#Cc1c(F)ccc2c1-c1cccnc1CC(=O)N2CC(=O)Nc1ccc(C(F)(F)F)cc1] | 2-(11-cyano-10-fluoro- 6-oxo-5H-pyrido[2,3- d][1]benzazepin-7-yl)- N-[4-(trifluoromethyl)- phenyl]acetamide | Starting from Intermediate N101. LC- MS (Method B2) m/z [M + H]⁺: 455.0; rt: 3.90 min; purity >99%. LC-MS (Method A2) m/z [M + H]⁺: 455.0; rt: 4.39 min; purity >99%. ¹H NMR (400 MHz, DMSO-d₆) δ 10.57 (s, 1H), 8.70 (dd, J = 4.9, 1.6 Hz, 1H), 8.35 (dd, J = 7.9, 1.8 Hz, 1H), 7.99 (dd, J = 9.3, 5.3 Hz, 1H), 7.76-7.72 (m, 3H), 7.67 (d, J = 8.9 Hz, 2H), 7.61 (dd, J = 7.9, 4.9 Hz, 1H), 4.56 (d, J = 16.8 Hz, 1H), 4.49 (d, J = 16.8 Hz, 1H), 3.92 (d, J = 12.7 Hz, 1H), 3.68 (d, J = 12.7 Hz, 1H). |
| #69 | 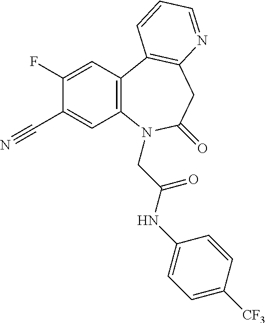 [SMILES: N#Cc1cc2c(cc1F)-c1cccnc1CC(=O)N2CC(=O)Nc1ccc(C(F)(F)F)cc1] | 2-(9-cyano-10-fluoro- 6-oxo-5H-pyrido[2,3- d][1]benzazepin-7-yl)- N-[4-(trifluoromethyl)- phenyl]acetamide | Starting from Intermediate N102. 5- amino-4-bromo-2-fluoro-benzonitrile and methyl 2-[3-(4,4,5,5-tetramethyl- 1,3,2-dioxaborolan-2-yl)-2-pyridyl] acetate. LC-MS (Method B2) m/z [M + H]⁺: 455.0; rt: 3.95 min; purity >99%. LC-MS (Method A2) m/z [M + H]⁺: 455.0; rt: 4.39 min; purity >9%. ¹H NMR (400 MHz, DMSO-d₆) δ 10.58 (s, 1H), 8.67 (dd, J = 4.8, 1.6 Hz, 1H), 8.22 (dd, J = 7.9, 1.6 Hz, 1H), 8.16 (d, J = 5.9 Hz, 1H), 7.92 (d, J = 10.2 Hz, 1H), 7.77 (d, J = 8.8 Hz, 2H), 7.67 (d, J = 8.8 Hz, 2H), 7.59 (dd, J = 7.9, 4.8 Hz, 1H), 4.63 (d, J = 16.8 Hz, 1H), 4.53 (d, J = 16.8 Hz, 1H), 3.82 (d, J = 12.7 Hz, 1H), 3.72 (d, J = 12.7 Hz, 1H). |

### Example #70: N-[4-(trifluoromethyl)phenyl]-2-(3,5,13-trimethyl-9-oxo-4,8,12-triazatricyclo[9.4.0.0^{2,7}]pentadeca-1(11),2(7),3,5,12,14-hexaen-8-yl)acetamide

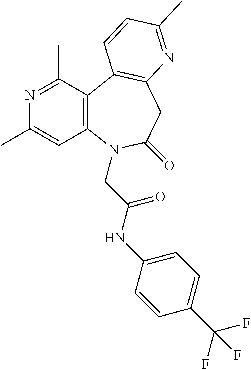 [SMILES: Cc1cc2c(c(C)n1)-c1ccc(C)nc1CC(=O)N2CC(=O)Nc1ccc(C(F)(F)F)cc1]

Under inert atmosphere, a mixture of example #36 (9 mg, 0.02 mmol), tertbutylperoxyacetate (20 μL, 0.06 mmol), trifluoroacetic acid (0.2 mL, 2.7 mmol) and (4,4′-di-t-butyl-2,2′-bipyridine)bis[3,5-difluoro-2-[5-trifluoromethyl-2-pyridinyl-kN)phenyl-kC]iridium(III) hexafluorophosphate (2 mg, 0.002 mmol) in dry acetonitrile (0.2 mL) was irradiated in a Pennoc reactor (450 nm; Fan=4000; Stirr=400; LED=100%) for 3 h. The crude mixture was purified by reverse phase chromatography (with basic elution) to afford the title product (0.2 mg, yield: 2%) as a white solid. LCMS (Method B3): [M+H]⁺ m/z: 455.17, rt: 4.06 min, purity: 92%. ¹H NMR (500 MHz, DMSO-d₆) δ 10.59 (s, 1H), 7.99 (d, J=7.0 Hz, 1H), 7.78 (d, J=7.5 Hz, 2H), 7.68 (d, J=7.5 Hz, 2H), 7.33 (d, J=7.0 Hz, 1H), 7.21 (s, 1H), 4.52 (s, 2H), 3.70 (d, J=12.5 Hz, 1H), 3.58 (d, J=12.5 Hz, 1H), 2.53 (s, 3H). 6H (2 CH₃) protons are merged in solvent peak.

### Example #71: 2-(5,13-dimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

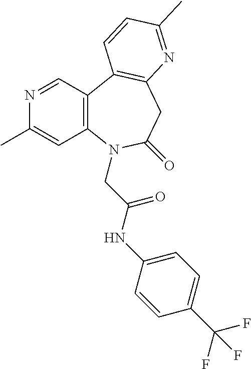 [SMILES: Cc1cc2c(cn1)-c1ccc(C)nc1CC(=O)N2CC(=O)Nc1ccc(C(F)(F)F)cc1]

The title compound (0.5 mg, yield: 5%) was obtained as a second product during the preparation of #70. LCMS (Method B4): [M+H]⁺ m/z: 441.15, rt: 3.66 min, purity: 92%, LCMS (Method A4): [M+H]⁺ m/z: 441.15, rt: 3.50 min, purity:>99%. ¹H NMR (500 MHz, DMSO-d₆) δ 10.66 (s, 1H), 8.69 (s, 1H), 8.07 (d, J=7.2 Hz, 1H), 7.80 (d, J=7.5 Hz, 2H), 7.69 (d, J=7.5 Hz, 2H), 7.40 (d, J=7.2 Hz, 1H), 7.36 (s, 1H), 4.63 (d, J=16.8 Hz, 1H), 4.55 (d, J=16.8 Hz, 1H), 3.70 (s, 2H), 2.54 (s, 6H).

### Example #72: 2-(1-ethyl-3-methyl-6-oxo-7H-pyrido[4,3-d][3]benzazepin-5-yl)-N-[4-(trifluoromethyl)phenyl]acetamide

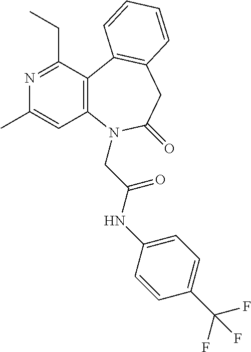 [SMILES: CCc1nc(C)cc2c1-c1ccccc1CC(=O)N2CC(=O)Nc1ccc(C(F)(F)F)cc1]

To a solution of propionic acid (3 μL, 0.04 mmol), N-hydroxyphthalimide (10 mg, 0.06 mmol) and 4-dimethylaminopyridine (0.25 mg, 0.002 mmol) in dry DMSO (0.2 mL) was added N,N′-diisopropylcarbodiimide (9 μL, 0.06 mmol) and the reaction mixture was irradiated in a Pennoc reactor (450 nm; Fan=2800; Stirr=400; LED=100%) at room temperature for 17 h to give a solution of (1,3-dioxoisoindolin-2-yl) propanoate (0.04 mmol). Then, a solution of example #14 (12 mg, 0.025 mmol) and TFA (4 μL, 0.050 mmol) in DMSO (0.2 mL) was transferred to the solution containing the (1,3-dioxoisoindolin-2-yl) propanoate reactant. 2,4,5,6-Tetra(9H-carbazol-9-yl)isophthalonitrile (0.2 mg, 0.0003 mmol) was added, the solution was degassed with Argon gas for 30 s, sealed, and irradiated in a Pennoc reactor (450 nm; Fan=1500; Stirr=400; LED=100%) for 3 h. The crude solution was purified by reverse phase chromatography (with basic elution) to afford the title compound (0.7 mg, yield: 6%). LCMS (Method B3): [M+H]⁺ m/z: 454.1, rt: 4.87 min, purity: 95%, LCMS (Method B3): [M+H]⁺ m/z: 454.1, rt: 3.73 min, purity: 95%. ¹H NMR (500 MHz, DMSO-d₆) δ 10.57 (s, 1H), 7.78 (d, J=8.5 Hz, 2H), 7.69 (d, J=8.5 Hz, 2H), 7.56 (d, J=6.6 Hz, 1H), 7.44-7.41 (m, 3H), 7.16 (s, 1H), 4.49 (d, J=16.8 Hz, 1H), 4.44 (d, J=16.8 Hz, 1H), 3.51 (d, J=11.9 Hz, 1H), 3.45 (d, J=11.9 Hz, 1H), 2.87-2.77 (m, 2H), 1.14 (t, J=7.5 Hz, 3H).

### Example #73: 2-(1-cyclopropyl-3-methyl-6-oxo-7H-pyrido[4,3-d][3]benzazepin-5-yl)-N-[4-(trifluoromethyl)phenyl]acetamide

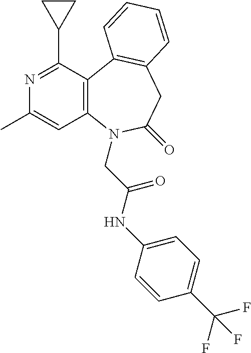 [SMILES: Cc1cc2c(c(C3CC3)n1)-c1ccccc1CC(=O)N2CC(=O)Nc1ccc(C(F)(F)F)cc1]

To a solution of example #14 (12 mg, 0.025 mmol), cyclopropanecarbonyl cyclopropanecarboperoxoate (13 mg, 0.073 mmol) and (4,4′-di-t-butyl-2,2′-bipyridine)bis[3,5-difluoro-2-[5-trifluoromethyl-2-pyridinyl-kN)phenyl-kC]iridium(III) hexafluorophosphate (2.3 mg, 0.002 mmol) in dry acetonitrile (0.13 mL) was added TFA (0.13 mL, 1.8 mmol). The resulting mixture was degassed with Argon and irradiated in a Pennoc reactor (450 nm; Fan=4000; Stirr=400; LED=100%) for 3 h. The crude solution was purified by reverse phase chromatography (with basic elution) to afford the title product (0.7 mg, yield: 6%). LCMS (Method B4): [M+H]⁺ m/z: 466.1, rt: 4.89 min, purity: 92%, LCMS (Method A4): [M+H]⁺ m/z: 466.1, rt: 4.64 min, purity: 95%. ¹H NMR (500 MHz, DMSO-d₆) δ 10.57 (s, 1H), 7.79 (d, J=8.4 Hz, 2H), 7.72-7.68 (m, 3H), 7.46-7.43 (m, 3H), 7.08 (s, 1H), 4.50 (d, J=16.8 Hz, 1H), 4.42 (d, J=16.8 Hz, 1H), 3.54 (d, J=12.4 Hz, 1H), 3.51 (d, J=12.4 Hz, 1H), 2.43 (s, 3H), 2.08-2.04 (m, 1H), 1.26-1.22 (m, 1H), 1.05-1.00 (m, 1H), 0.97-0.93 (m, 1H), 0.79-0.76 (m, 1H).

### Example #74: 2-(14-methoxy-12-methyl-8-oxo-4,5,6,9,13-pentazatricyclo[8.4.0.0^{2,6}]tetradeca-1(1),2,4,11,13-pentaen-9-yl)-N-[4-(trifluoromethyl)phenyl]acetamide

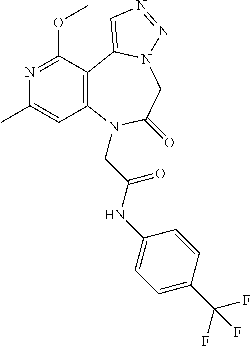 [SMILES: COc1nc(C)cc2c1-c1cnnn1CC(=O)N2CC(=O)Nc1ccc(C(F)(F)F)cc1]

The title product was prepared according to general procedure 1, starting from Intermediate N24 and 2-chloro-N-(4-trifluoromethyl-phenyl)acetamide. The title compound was obtained as a white solid (12 mg, yield: 30%). LC-MS (Method B5) m/z [M+H]⁺: 447.2; rt: 2.10 min, purity: 99%. ¹H NMR (400 MHz, DMSO-d₆) δ 10.63 (s, 1H), 8.13 (s, 1H), 7.78 (d, J=8.5 Hz, 2H), 7.68 (d, J=8.6 Hz, 2H), 7.07 (s, 1H), 4.60 (s, 2H), 3.98 (s, 3H), 3.30 (s, 2H), 2.48 (s, 3H).

### Example #75: 2-(3-methoxy-5-methyl-9-oxo-4,8,12-triazatricyclo[9.4.0.0^{2,7}]pentadeca-1(11),2,4,6,12,14-hexaen-8-yl)-N-[4-(trifluoromethyl)phenyl]acetamide

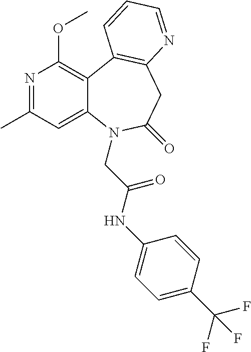 [SMILES: COc1nc(C)cc2c1-c1cccnc1CC(=O)N2CC(=O)Nc1ccc(C(F)(F)F)cc1]

The title product was prepared according to general procedure 1, starting from Intermediate N25 and 2-chloro-N-(4-chloro-3-fluoro-phenyl)acetamide (CAS 895641-02-8). Purification by flash chromatography on silica gel (using 0 to 10% MeOH in DCM as eluent) afforded the title compound as a white solid (260 mg, yield: 78%). LC-MS (Method B5) m/z [M+H]⁺: 438.1; rt: 4.75 min, purity: 99%. ¹H NMR (400 MHz, DMSO-d₆) δ 10.57 (s, 1H), 8.53 (dd, J=4.8, 1.7 Hz, 1H), 8.17 (dd, J=7.9, 1.7 Hz, 1H), 7.78 (d, J=8.5 Hz, 2H), 7.68 (d, J=8.6 Hz, 2H), 7.45 (dd, J=8.0, 4.8 Hz, 1H), 7.03 (s, 1H), 4.53 (s, 2H), 3.90 (s, 3H), 3.74 (d, J=12.4 Hz, 1H), 3.68 (d, J=12.4 Hz, 1H), 2.46 (s, 3H).

### Example #76: 2-(3-hydroxy-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

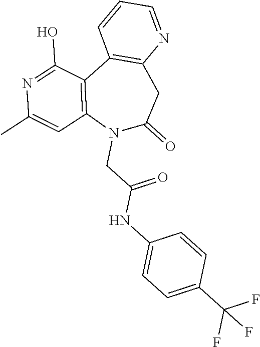 [SMILES: Cc1cc2c(c(O)n1)-c1cccnc1CC(=O)N2CC(=O)Nc1ccc(C(F)(F)F)cc1]

Example #75 (20.0 mg, 0.044 mmol) and potassium iodide (22 mg, 0.131 mmol) were suspended in dry acetonitrile (2 mL) and trimethylsilyl chloride (0.02 mL, 0.131 mmol) was then added. The reaction mixture was stirred at 80° C. for 2 h and then cooled to room temperature. The reaction mixture was concentrated under vacuum and the residue was partitioned between ethyl acetate (20 mL) and water (20 mL). The layers were separated, and the organic phase was washed with saturated aqueous sodium thiosulfate (20 mL). The organic phase was filtered through a hydrophobic frit and concentrated under vacuum. The residue was purified by flash chromatography on silica gel (using 0 to 10% MeOH in DCM as eluent) to give the title compound (15 mg, yield: 77%) as a white solid. LC-MS (Method B5) m/z: [M+H]⁺: 443.2; rt: 1.66 min; purity: 99%. ¹H NMR (400 MHz, DMSO-d₆) δ 11.95 (s, 1H), 10.57 (s, 1H), 8.49 (dd, J=4.8, 1.7 Hz, 1H), 8.30 (dd, J=8.0, 1.7 Hz, 1H), 7.78 (d, J=8.5 Hz, 2H), 7.68 (d, J=8.5 Hz, 2H), 7.40 (dd, J=8.0, 4.8 Hz, 1H), 6.22 (s, 1H), 4.44 (s, 2H), 3.70 (s, 2H), 2.24 (s, 3H).

The following Examples #77-83 have been prepared according to general procedure 1, starting respectively from commercially available 2-chloro-N-(4-trifluoro methylphenyl)acetamide (CAS 2707-23-5), 2-chloro-N-(4-cyclopropylphenyl)acetamide (Intermediate N129), 2-chloro-N-(4-cyanophenyl)acetamide (CAS 114807-80-6), 2-chloro-N-[6-(trifluoromethyl)-3-pyridyl]acetamide (CAS 1443679-86-4) and 2-chloro-N-(4-difluromethylphenyl)acetamide (CAS 872533-93-2) and other specific intermediates referenced in the table below.

| Ex | Structure Example | Example Name | Protocolumn and characterization |
|---|---|---|---|
| #77 | 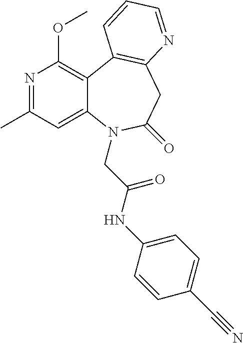 [SMILES: COc1nc(C)cc2c1-c1cccnc1CC(=O)N2CC(=O)Nc1ccc(C#N)cc1] | N-(4-cyanophenyl)-2-(3- methoxy-5-methyl-9- oxo-4,8,12-triazatricyclo [9.4.0.0^{2,7}]pentadeca- 1(11),2,4,6,12,14- hexaen-8-yl)acetamide | Starting from 3-methoxy-5-methyl-4,8, 12-triazatricyclo[9.4.0.0^{2,7}]pentadeca- 1(11),2(7),3,5,12,14-hexaen-9-one (Intermediate N25). LC-MS (Method B5′): m/z [M + H]⁺ 414.2; rt: 1.88 min; purity: 99%. ¹H NMR (400 MHz, DMSO-d₆) δ 10.65 (s, 1H), 8.53 (dd, J = 4.8, 1.7 Hz, 1H), 8.17 (dd, J = 8.0, 1.7 Hz, 1H), 7.82-7.71 (m, 4H), 7.44 (dd, J = 8.0, 4.8 Hz, 1H), 7.02 (s, 1H), 4.53 (s, 2H), 3.90 (s, 3H), 3.77-3.66 (m, 2H), 2.46 (s, 3H). |
| #78 | 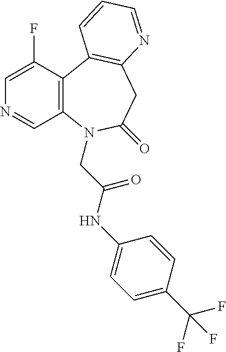 [SMILES: O=C(CN1C(=O)Cc2ncccc2-c2c(F)cncc21)Nc1ccc(C(F)(F)F)cc1] | 2-(3-fluoro-9-oxo-5,8,12- triazatricyclo[9.4.0.0^{2,7}] pentadeca-1(11),2(7),3, 5,12,14-hexaen-8-yl)-N- [4-(trifluoromethyl)- phenyl]acetamide | Starting from Intermediate N103. LC- MS (Method B4) m/z [M + H]⁺: 431.0; rt: 3.93 min; purity: 96%. LC-MS (Method A4) m/z [M + H]⁺: 431.0; rt: 4.02 min; purity: 96%. ¹H NMR (400 MHz, DMSO-d₆) δ 10.60 (s, 1H), 8.75- 8.64 (m, 3H), 8.23 (s, 1H), 7.75 (d, J = 8.5 Hz, 2H), 7.67 (d, J = 8.5 Hz, 2H), 7.58 (dd, J = 8.0, 4.8 Hz, 1H), 4.72 (d, J = 16.8 Hz, 1H), 4.63 (d, J = 16.8 Hz, 1H), 3.91 (d, J = 12.7 Hz, 1H), 3.73 (d, J = 12.7 Hz, 1H). |
| #79 | 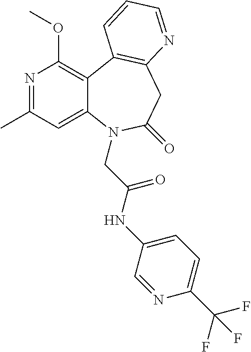 [SMILES: COc1nc(C)cc2c1-c1cccnc1CC(=O)N2CC(=O)Nc1ccc(C(F)(F)F)nc1] | 2-(3-methoxy-5-methyl- 9-oxo-4,8,12-triazatri- cyclo[9.4.0.0^{2,7}]penta- deca-1(11),2,4,6,12,14- hexaen-8-yl)-N-[6-(tri- fluoromethyl)-3-pyridyl] acetamide | Starting from 3-methoxy-5-methyl- 4,8,12-triazatricyclo[9.4.0.0^{2,7}]penta- deca-1(11),2(7),3,5,12,14-hexaen-9- one (Intermediate N25). LC-MS (Method B5'): m/z [M + H]⁺: 458.2; rt: 1.97 min; purity: 96%. ¹H NMR (400 MHz, DMSO-d₆) δ 10.85 (s, 1H), 8.85 (d, J = 2.4 Hz, 1H), 8.53 (dd, J = 4.8, 1.7 Hz, 1H), 8.30 (dd, J = 8.6, 2.4 Hz, 1H), 8.17 (dd, J = 8.0, 1.7 Hz, 1H), 7.87 (d, J = 8.6 Hz, 1H), 7.44 (dd, J = 8.0, 4.8 Hz, 1H), 7.02 (s, 1H), 4.55 (s, 2H), 3.90 (s, 3H), 3.72 (d, J = 12.4 Hz, 1H), 3.70 (d, J = 12.4 Hz, 1H) 2.46 (s, 3H). |
| #80 | 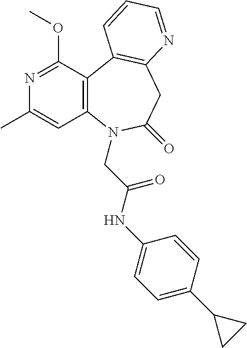 [SMILES: COc1nc(C)cc2c1-c1cccnc1CC(=O)N2CC(=O)Nc1ccc(C2CC2)cc1] | N-(4-cyclopropylphen- yl)-2-(3-methoxy-5- methyl-9-oxo-4,8,12- triazatricyclo[9.4.0.0^{2.7}] pentadeca-1(11),2(7),3, 5,12,14-hexaen-8-yl)- acetamide | Starting from 3-methoxy-5-methyl- 4,8,12-triazatricyclo[9.4.0.0^{2,7}]penta- deca-1(11),2(7),3,5,12,14-hexaen-9- one (Intermediate N25). LC-MS (Method A7): m/z [M + H]⁺: 429.2; rt: 2.08 min; purity: 99%. ¹H NMR (400 MHz, DMSO-d₆) δ 10.08 (s, 1H), 8.52 (dd, J = 4.8, 1.7 Hz, 1H), 8.16 (dd, J = 8.0, 1.7 Hz, 1H), 7.48- 7.38 (m, 3H), 7.00 (d, J = 8.8 Hz, 3H), 4.56-4.36 (m, 2H), 3.89 (s, 3H), 3.78- 3.61 (m, 2H), 2.45 (s, 3H), 1.86 (tt, J = 8.4, 5.1 Hz, 1H), 0.94-0.86 (m, 2H), 0.65-0.56 (m, 2H). |
| #81 | 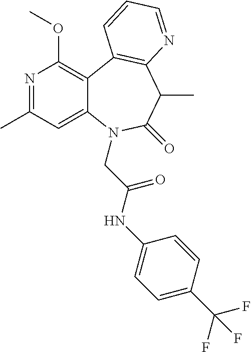 [SMILES: COc1nc(C)cc2c1-c1cccnc1C(C)C(=O)N2CC(=O)Nc1ccc(C(F)(F)F)cc1] | 2-(3-methoxy-5,10- dimethyl-9-oxo-4,8,12- triazatricyclo[9.4.0.0^{2,7}] pentadeca-1(11),2,4,6, 12,14-hexaen-8-yl)-N- [4-(trifluoromethyl)- phenyl]acetamide | Starting from Intermediate N104. LC- MS (Method A7): m/z [M + H]⁺: 471.2; rt: 2.38 min; purity: 98%. ¹H NMR (400 MHz, DMSO-d₆) δ 10.56 (s, 1H), 8.59 (dd, J = 4.7, 1.7 Hz, 1H), 8.15 (dd, J = 8.0, 1.7 Hz, 1H), 7.77 (d, J = 8.5 Hz, 2H), 7.68 (d, J = 8.7 Hz, 2H), 7.43 (dd, J = 7.9, 4.7 Hz, 1H), 7.04 (s, 1H), 4.52 (s, 2H), 3.91 (s, 3H), 3.65 (q, J = 6.6 Hz, 1H), 2.47 (s, 3H), 1.48 (d, J = 6.6 Hz, 3H). |
| #82 | 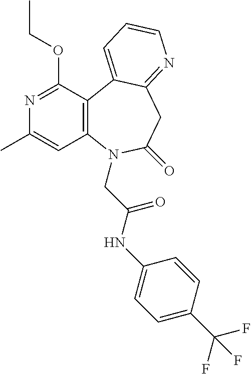 [SMILES: CCOc1nc(C)cc2c1-c1cccnc1CC(=O)N2CC(=O)Nc1ccc(C(F)(F)F)cc1] | 2-(3-ethoxy-5-methyl-9- oxo-4,8,12-triazatri- cyclo[9.4.0.0^{2,7}]penta- deca-1(15),2(7),3,5,11, 13-hexaen-8-yl)-N-[4- (trifluoromethyl)phenyl] acetamide | Starting from Intermediate N105. LC- MS (Method A7): m/z [M + H]: 471.2; rt: 2.33 min, purity: 99%. ¹H NMR (400 MHz, DMSO-d₆) δ 10.56 (s, 1H), 8.52 (dd, J = 4.8, 1.7 Hz, 1H), 8.19 (dd, J = 8.0, 1.7 Hz, 1H), 7.77 (d, J = 8.5 Hz, 2H), 7.68 (d, J = 8.5 Hz, 2H), 7.44 (dd, J = 8.0, 4.8 Hz, 1H), 7.00 (s, 1H), 4.51 (s, 2H), 4.50-4.43 (m, 1H), 4.27 (dq, J = 10.7, 7.0 Hz, 1H), 3.70 (q, J = 12.3 Hz, 2H), 2.44 (s, 3H), 1.30 (t, J = 7.0 Hz, 3H). |
| #83 | 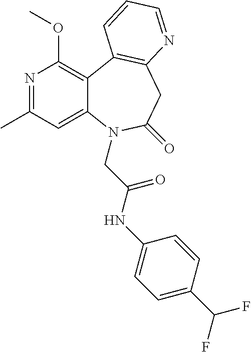 [SMILES: COc1nc(C)cc2c1-c1cccnc1CC(=O)N2CC(=O)Nc1ccc(C(F)F)cc1] | N-[4-(difluoromethyl)- phenyl]-2-(3-methoxy- 5-methyl-9-oxo-4,8,12- triazatricyclo[9.4.0.0^{2,7}] pentadeca-1(11),2,4,6, 12,14-hexaen-8-yl)acet- amide | Starting from 3-methoxy-5-methyl- 4,8,12-triazatricyclo[9.4.0.0^{2,7}]penta- deca-1(11),2(7),3,5,12,14-hexaen-9- one (Intermediate N25). LC-MS (Method B5′): m/z [M + H]⁺: 439.2; rt: 2.01 min; purity: >99%. ¹H NMR (400 MHz, DMSO-d₆) δ 10.42 (s, 1H), 8.52 (dd, J = 4.8, 1.7 Hz, 1H), 8.16 (dd, J = 8.0, 1.7 Hz, 1H), 7.69 (d, J = 8.4 Hz, 2H), 7.51 (d, J = 8.4 Hz, 2H), 7.44 (dd, J = 7.9, 4.8 Hz, 1H), 7.13- 6.80 (m, 2H), 4.51 (s, 2H), 3.89 (s, 3H), 3.73 (d, J = 12.3 Hz, 1H), 3.67 (d, J = 12.3 Hz, 1H), 2.45 (s, 3H). |

### Example #84: 2-(3-fluoro-5,10-dimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

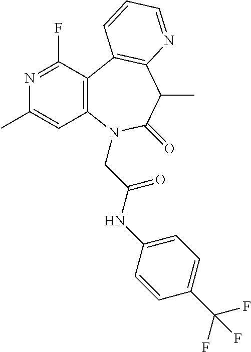 [SMILES: Cc1cc2c(c(F)n1)-c1cccnc1C(C)C(=O)N2CC(=O)Nc1ccc(C(F)(F)F)cc1]

The title product was prepared according to general procedure 1, starting from Intermediate N23 (39 mg, 0.14 mmol) and 2-chloro-N-(4-trifluoromethylphenyl)acetamide (CAS 2707-23-5). Purification by preparative HPLC (acidic elution) afforded the title product as a white solid (37 mg, yield: 55%). LC-MS (Method B3) m/z [M+H]⁺: 459.0; rt: 4.62 min; purity: 99%. LC-MS (Method A3) m/z [M+H]⁺: 459.0; rt: 5.00 min; purity: 98%. ¹H NMR (400 MHz, DMSO-d₆) δ 10.60 (s, 1H), 8.67 (dd, J=4.8, 1.8 Hz, 1H), 8.13 (ddd, J=7.9, 4.8, 1.8 Hz, 1H), 7.76 (d, J=8.8 Hz, 2H), 7.68 (d, J=8.8 Hz, 2H), 7.50 (dd, J=7.9, 4.8 Hz, 1H), 7.38 (s, 1H), 4.60 (s, 2H), 3.79 (q, J=6.6 Hz, 1H), 2.50 (s, 3H), 1.49 (d, J=6.6 Hz, 3H).

The following Examples #85-93 have been prepared general procedure 1, starting respectively from commercially available 2-chloro-N-(4-trifluoromethylphenyl)acetamide (CAS 2707-23-5), 2-chloro-N-(3-fluoro-4-chlorophenyl)acetamide (CAS 895641-02-8), and 2-chloro-N-(4-cyanophenyl)acetamide (CAS 114807-80-6), and other specific intermediates referenced in the table below.

| Ex | Structure Example | Example Name | Protocolumn and characterization |
|---|---|---|---|
| #85 | 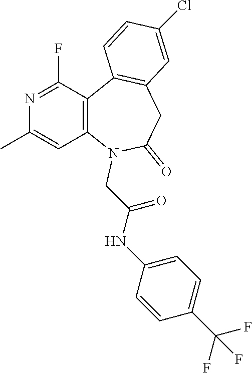 [SMILES: Cc1cc2c(c(F)n1)-c1ccc(Cl)cc1CC(=O)N2CC(=O)Nc1ccc(C(F)(F)F)cc1] | 2-(9-chloro-1-fluoro- 3-methyl-6-oxo-7H- pyrido[4,3-d][3] benzazepin-5-yl)-N- [4-(trifluoromethyl)- phenyl]acetamide | Starting from Intermediate N118. LC-MS (Method B3) m/z [M + H]⁺: 478.0; rt: 5.21 min; purity >99%. LC- MS (Method A3) m/z [M + H]⁺: 478.0; rt: 5.77 min; purity: 99%. ¹H NMR (400 MHz, DMSO-d₆) δ 10.60 (s, 1H), 7.78 (d, J = 8.7 Hz, 2H), 7.74-7.67 (m, 3H), 7.64 (d, J = 2.4 Hz, 1H), 7.52 (dd, J = 8.4, 2.4 Hz, 1H), 7.33 (s, 1H), 4.57 (d, J = 16.8 Hz, 1H), 4.54 (d, J = 16.8 Hz, 1H), 3.67 (d, J = 12.8 Hz, 1H), 3.59 (d, J = 12.8 Hz, 1H), 2.48 (s, 3H). |
| #86 | 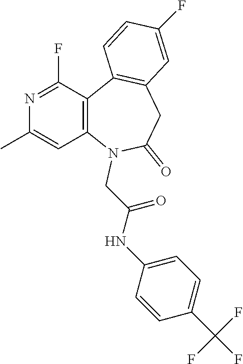 [SMILES: Cc1cc2c(c(F)n1)-c1ccc(F)cc1CC(=O)N2CC(=O)Nc1ccc(C(F)(F)F)cc1] | 2-(1,9-difluoro-3- methyl-6-oxo-7H- pyrido[4,3-d][3] benzazepin-5-yl)-N- [4-(trifluorometh- yl)phenyl]acetamide | Starting from Intermediate N119. LC- MS (Method B3) m/z [M + H]⁺: 462.1; rt: 5.05 min; purity >99%. LC-MS (Method A3) m/z [M + H]⁺: 462.1; rt: 5.50 min; purity: 99%. ¹H NMR (400 MHz, DMSO-d₆) δ 10.59 (s, 1H), 7.85- 7.71 (m, 3H), 7.68 (d, J = 9.2 Hz, 2H), 7.42 (dd, J = 9.1, 2.8 Hz, 1H), 7.35- 7.26 (m, 2H), 4.58 (d, J = 16.8 Hz, 1H), 4.53 (d, J = 16.8 Hz, 1H), 3.64 (d, J = 12.8 Hz, 1H), 3.58 (d, J = 12.8 Hz, 1H), 2.47 (s, 3H). |
| #87 | 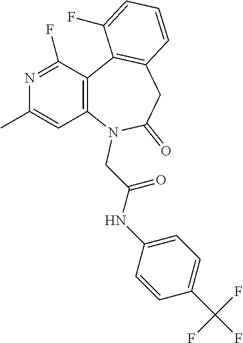 [SMILES: Cc1cc2c(c(F)n1)-c1c(F)cccc1CC(=O)N2CC(=O)Nc1ccc(C(F)(F)F)cc1] | 2-(1,11-difluoro-3- methyl-6-oxo-7H- pyrido[4,3-d][3] benzazepin-5-yl)-N- [4-(trifluoromethyl)- phenyl]acetamide | Starting from Intermediate N120. LC- MS (Method B3) m/z [M + H]⁺: 462.1; rt: 4.81 min; purity >99%. LC-MS (Method A3) m/z [M + H]⁺: 462.1; rt: 5.45 min; purity >99%. ¹H NMR (400 MHz, DMSO-d₆) δ 10.61 (s, 1H), 7.78 (d, J = 8.8 Hz, 2H), 7.68 (d, J = 8.8 Hz, 2H), 7.55 (td, J = 8.0, 5.5 Hz, 1H), 7.38-7.29 (m, 3H), 4.59 (s, 2H), 3.68 (d, J = 12.8 Hz, 1H), 3.64 (d, J = 12.8 Hz, 1H), 2.49 (s, 3H). |
| #88 | 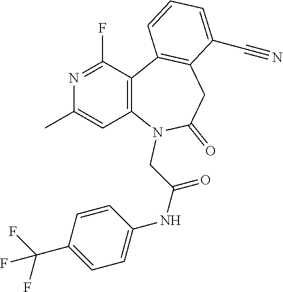 [SMILES: Cc1cc2c(c(F)n1)-c1cccc(C#N)c1CC(=O)N2CC(=O)Nc1ccc(C(F)(F)F)cc1] | 2-(8-cyano-1-fluoro- 3-methyl-6-oxo-7H- pyrido[4,3-d][3] benzazepin-5-yl)-N- [4-(trifluoromethyl)- phenyl]acetamide | Starting from Intermediate N121. LC- MS (Method A4) m/z [M + H]⁺: 469.0; rt: 4.71 min; purity: 96%. ¹H NMR (400 MHz, DMSO-d₆) δ 10.60 (s, 1H), 8.09-8.00 (m, 2H), 7.82 (d, J = 8.8 Hz, 1H), 7.76 (d, J = 8.7 Hz, 2H), 7.72-7.62 (m, 2H), 7.38 (s, 1H), 4.61 (s, 2H), 3.89 (d, J = 13.1 Hz, 1H), 3.81 (d, J = 13.1 Hz, 1H), 2.50 (s, 3H). |
| #89 | 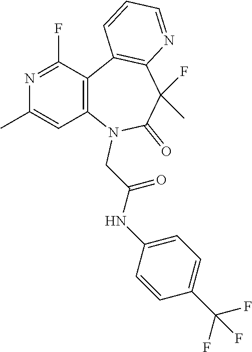 [SMILES: Cc1cc2c(c(F)n1)-c1cccnc1C(C)(F)C(=O)N2CC(=O)Nc1ccc(C(F)(F)F)cc1] | 2-(3,10-difluoro-5, 10-dimethyl-9-oxo- 4,8,12-triazatricyclo [9.4.0.0^{2,7}]pentadeca- 1(11),2(7),3,5,12,14- hexaen-8-yl)-N-[4- (trifluoromethyl)- phenyl]acetamide | Starting from Intermediate N122. LC- MS (Method A2) m/z [M + H]⁺: 477.0; rt: 4.87 min; purity: 95%. ¹H NMR (400 MHz, DMSO-d₆) δ 10.66 (s, 1H), 8.75 (d, J = 3.8 Hz, 1H), 8.32 (d, J = 11.8 Hz, 1H), 7.76 (d, J = 8.8 Hz, 2H), 7.74-7.64 (m, 3H), 7.41 (s, 1H), 4.73 (d, J = 16.8 Hz, 1H), 4.69 (d, J = 16.8 Hz, 1H), 2.50 (s, 3H), 2.03 (d, J = 24.5 Hz, 3H). |
| #90 | 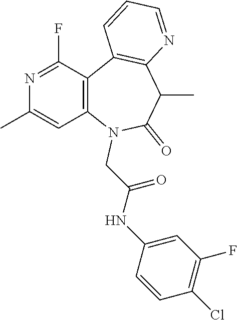 [SMILES: Cc1cc2c(c(F)n1)-c1cccnc1C(C)C(=O)N2CC(=O)Nc1ccc(Cl)c(F)c1] | N-(4-chloro-3-fluoro- phenyl)-2-(3-fluoro- 5,10-dimethyl-9-oxo- 4,8,12-triazatricyclo [9.4.0.0^{2,7}]pentadeca- 1(11),2(7),3,5,12,14- hexaen-8-yl)acet- amide | Starting from 3-fluoro-5,10-dimethyl- 4,8,12-triazatricyclo[9.4.0.0^{2,7}]penta- deca-1(11),2(7),3,5,12,14-hexaen-9-one (Intermediate N23). LC-MS (Method B2) m/z [M + H]⁺: 443.0; rt: 4.44 min; purity: 97%. LC-MS (Method A2) m/z [M + H]⁺: 443.0; rt: 4.47 min; purity: 95%. ¹H NMR (400 MHz, DMSO-d₆) δ 10.58 (s, 1H), 8.67 (dd, J = 4.8, 1.8 Hz, 1H), 8.12 (ddd, J = 7.9, 4.6, 1.6 Hz, 1H), 7.73 (dd, J = 11.9, 2.4 Hz, 1H), 7.57-7.46 (m, 2H), 7.36 (s, 1H), 7.31 (dd, J = 8.2, 1.9 Hz, 1H), 4.56 (s, 2H), 3.78 (q, J = 6.7 Hz, 1H), 1.49 (d, J = 6.7 Hz, 3H). |
| #91 | 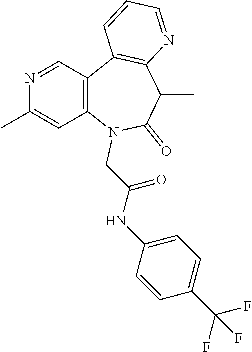 [SMILES: Cc1cc2c(cn1)-c1cccnc1C(C)C(=O)N2CC(=O)Nc1ccc(C(F)(F)F)cc1] | 2-(5,10-dimethyl-9- oxo-4,8,12-triazatri- cyclo[9.4.0.0^{2,7}]penta- deca-1(11),2(7),3,5, 12,14-hexaen-8-yl)- N-[4-(trifluorometh- yl)phenyl]acetamide | Starting from Intermediate N123. LC- MS (Method B2) m/z [M + H]⁺: 441.0; rt: 4.06 min; purity >99%. LC-MS (Method A2) m/z [M + H]⁺: 441.0; rt: 3.42 min; purity: 97%. ¹H NMR (400 MHz, DMSO-d₆) δ 10.53 (s, 1H), 8.66 (s, 1H), 8.61 (dd, J = 4.8, 1.8 Hz, 1H), 8.09 (dd, J = 7.8, 1.8 Hz, 1H), 7.71 (d, J = 8.9 Hz, 2H), 7.61 (d, J = 8.9 Hz, 2H), 7.46 (dd, J = 7.8, 4.8 Hz, 1H), 7.31 (s, 1H), 4.57 (d, J = 16.9 Hz, 1H), 4.50 (d, J = 16.9 Hz, 1H), 3.58 (q, J = 6.7 Hz, 1H), 2.49 (s, 3H), 1.43 (d, J = 6.7 Hz, 3H). |
| #92 | 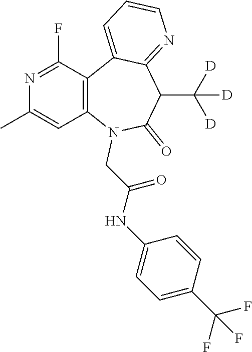 [SMILES: [2H]C([2H])([2H])C1C(=O)N(CC(=O)Nc2ccc(C(F)(F)F)cc2)c2cc(C)nc(F)c2-c2cccnc21] | 2-[3-fluoro-5-methyl- 9-oxo-10-(trideuterio- methyl)-4,8,12-triaza- tricyclo[9.4.0.0^{2,7}] pentadeca-1(15),2(7), 3,5,11,13-hexaen-8- yl]-N-[4-(trifluoro- methyl)phenyl]acet- amide | Starting from Intermediate N124. LC- MS (Method A2) m/z [M + H]⁺: 462.4; rt: 4.53 min; purity: 97%. ¹H NMR (400 MHz, DMSO-d₆) δ 10.60 (s, 1H), 8.67 (dd, J = 4.8, 1.8 Hz, 1H), 8.13 (ddd, J = 7.9, 4.8, 1.8 Hz, 1H), 7.77 (d, J = 8.8 Hz, 2H), 7.68 (d, J = 8.8 Hz, 2H), 7.50 (dd, J = 7.9, 4.8 Hz, 1H), 7.38 (s, 1H), 4.60 (s, 2H), 3.77 (s, 1H), 2.52 (s, 3H). |
| #93 | 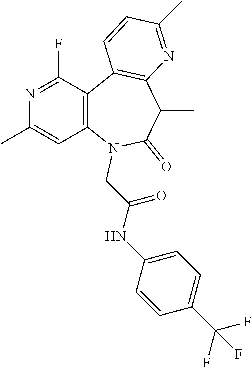 [SMILES: Cc1cc2c(c(F)n1)-c1ccc(C)nc1C(C)C(=O)N2CC(=O)Nc1ccc(C(F)(F)F)cc1] | 2-(3-fluoro-5,10,13- trimethyl-9-oxo-4,8, 12-triazatricyclo [9.4.0.0^{2,7}]pentadeca- 1(11),2(7),3,5,12,14- hexaen-8-yl)-N-[4- (trifluoromethyl)- phenyl]acetamide | Starting from Intermediate N125. LC- MS (Method A2) m/z [M + H]⁺: 473.1; rt: 4.91 min; purity: 96%. ¹H NMR (400 MHz, DMSO-d₆) δ 10.59 (s, 1H), 7.99 (dd, J = 8.0, 4.6 Hz, 1H), 7.77 (d, J = 8.8 Hz, 2H), 7.68 (d, J = 8.8 Hz, 2H), 7.40-7.31 (m, 2H), 4.59 (s, 2H), 3.70 (q, J = 6.6 Hz, 1H), 2.55 (s, 3H), 2.50 (s, 3H), 1.47 (d, J = 6.6 Hz, 3H). |

### Example #94: 2-(3-fluoro-10-hydroxy-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

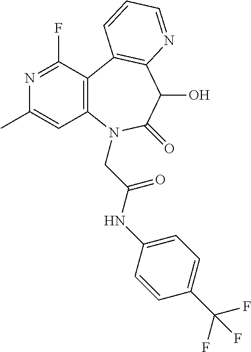 [SMILES: Cc1cc2c(c(F)n1)-c1cccnc1C(O)C(=O)N2CC(=O)Nc1ccc(C(F)(F)F)cc1]

The title product was prepared according to general procedure 1, starting from Intermediate N22 (108 mg, 0.37 mmol) and 2-chloro-N-(4-trifluoromethylphenyl)acetamide (CAS 2707-23-5). Purification by preparative HPLC (with acidic elution) afforded the title product as a beige solid (45 mg, yield: 25%). LC-MS (Method B3) m/z [M+H]⁺: 461.0; rt: 4.15 min; purity: 97%. LC-MS (Method A3) m/z [M+H]⁺: 461.0; rt: 4.40 min; purity: 96%. ¹H NMR (400 MHz, DMSO-d₆) δ 10.61 (s, 1H), 8.71 (dd, J=4.8, 1.6 Hz, 1H), 8.17 (ddd, J=7.9, 4.8, 1.6 Hz, 1H), 7.77 (d, J=8.9 Hz, 2H), 7.68 (d, J=8.9 Hz, 2H), 7.57 (dd, J=7.9, 4.8 Hz, 1H), 7.39 (s, 1H), 5.69 (d, J=8.7 Hz, 1H), 5.18 (d, J=8.7 Hz, 1H), 4.62 (s, 2H). Proton for OH not observed.

### Example #95: 2-(1-fluoro-7-hydroxy-3-methyl-6-oxo-7H-pyrido[4,3-d][3]benzazepin-5-yl)-N-[4-(trifluoromethyl)phenyl]acetamide

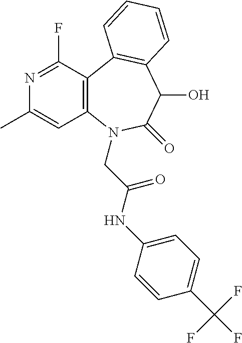 [SMILES: Cc1cc2c(c(F)n1)-c1ccccc1C(O)C(=O)N2CC(=O)Nc1ccc(C(F)(F)F)cc1]

The title product was prepared according to general procedure 1 starting from Intermediate N126 and 2-chloro-N-(4-trifluoromethylphenyl)acetamide (CAS 2707-23-5). LC-MS (Method B1) m/z [M+H]⁺: 460.1; rt: 2.66 min; purity: 96%. LC-MS (Method A1) m/z [M+H]⁺: 460.2; rt: 2.67 min; purity: 95%. ¹H NMR (400 MHz, DMSO-d₆) δ 10.62 (s, 1H), 7.80 (d, J=8.7 Hz, 2H), 7.72-7.62 (m, 4H), 7.55 (t, J=7.6 Hz, 1H), 7.45 (t, J=7.6 Hz, 1H), 7.33 (s, 1H), 6.12 (d, J=5.9 Hz, 1H), 4.98 (d, J=5.9 Hz, 1H), 4.62 (d, J=16.9 Hz, 1H), 4.56 (d, J=16.9 Hz, 1H), 2.50 (s, 3H).

### Example #96: 2-(10-hydroxy-3-methoxy-5-methyl-9-oxo-4,8,12-triazatricyclo[9.4.0.0^{2,7}]pentadeca-1(11),2,4,6,12,14-hexaen-8-yl)-N-[4-(trifluoromethyl)phenyl]acetamide

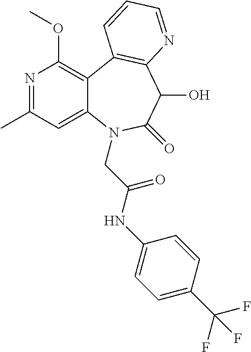 [SMILES: COc1nc(C)cc2c1-c1cccnc1C(O)C(=O)N2CC(=O)Nc1ccc(C(F)(F)F)cc1]

The title product was prepared according to general procedure 1 starting from Intermediate N127 and 2-chloro-N-(4-trifluoromethylphenyl)acetamide (CAS 2707-23-5). LC-MS (Method A7): m/z [M+H]⁺: 473.2; rt: 2.38 min; purity: 95%. ¹H NMR (400 MHz, DMSO-d₆) δ 10.57 (s, 1H), 8.63 (dd, J=4.8, 1.6 Hz, 1H), 8.20 (dd, J=7.9, 1.6 Hz, 1H), 7.76 (d, J=8.6 Hz, 2H), 7.67 (d, J=8.7 Hz, 2H), 7.50 (dd, J=8.0, 4.6 Hz, 1H), 7.04 (s, 1H), 5.56 (d, J=8.5 Hz, 1H), 5.01 (d, J=8.4 Hz, 1H), 4.54 (s, 2H), 3.91 (s, 3H), 2.46 (s, 3H).

### Example #97: 2-(3-chloro-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

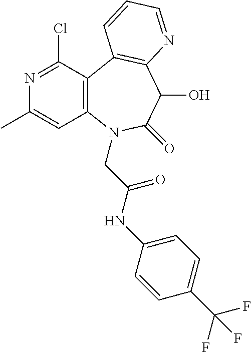 [SMILES: Cc1cc2c(c(Cl)n1)-c1cccnc1C(O)C(=O)N2CC(=O)Nc1ccc(C(F)(F)F)cc1]

A solution of example #76 (106 mg, 0.228 mmol) and POCl₃ (3.0 mL, 32.8 mmol) was heated at reflux overnight. The reaction mixture was cooled to room temperature and POCl₃ was removed under vacuum. A 2 M aqueous solution of NaOH (50 mL) was added and the mixture was extracted with EtOAc (50 mL). The organic phase was washed with water (50 mL), brine (50 mL), dried over Na₂SO₄, filtered and concentrated under vacuum. The residue was purified by flash chromatography on silica gel (using 0 to 5% MeOH in DCM as eluent) to give the title compound as a beige solid (5 mg, yield: 4%). LC-MS (Method B5) m/z: [M+H]⁺: 461.2/463.1; rt: 2.06 min; purity: 93%. ¹H NMR (400 MHz, DMSO-d₆) δ 10.59 (s, 1H), 8.61 (dd, J=4.8, 1.7 Hz, 1H), 8.26 (dd, J=8.0, 1.7 Hz, 1H), 7.77 (d, J=8.6 Hz, 2H), 7.68 (d, J=8.6 Hz, 2H), 7.51 (dd, J=8.0, 4.8 Hz, 1H), 7.46 (s, 1H), 4.61 (d, J=16.9 Hz, 1H), 4.54 (d, J=16.9 Hz, 1H), 3.91 (d, J=12.6 Hz, 1H), 3.68 (d, J=12.6 Hz, 1H), 2.53 (s, 3H).

### Example #98: 2-(15-methoxy-13-methyl-9-oxo-4,6,10,14-tetrazatricyclo[9.4.0.0^{2,7}]pentadeca-1(15),2(7),3,5,11,13-hexaen-10-yl)-N-[4-(trifluoromethyl)phenyl]acetamide

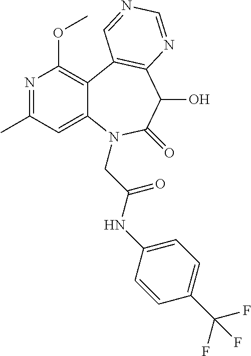 [SMILES: COc1nc(C)cc2c1-c1cncnc1C(O)C(=O)N2CC(=O)Nc1ccc(C(F)(F)F)cc1]

The title product was prepared according to general procedure 1, starting from Intermediate N26 and 2-chloro-N-(4-trifluoromethyl-phenyl)acetamide. Purification by flash chromatography on silica gel (using 0 to 100% ethyl acetate in iso-hexane as eluent) afforded the title compound (20 mg, yield: 61%) as a light yellow solid. LC-MS (Method B5) m/z [M+H]⁺: 458.2; rt 2.13 min; purity 96%. ¹H NMR (400 MHz, DMSO-d₆) δ 10.58 (s, 1H), 9.19 (s, 1H), 9.12 (s, 1H), 7.76 (d, J=8.5 Hz, 2H), 7.68 (d, J=8.5 Hz, 2H), 7.07 (s, 1H), 4.56 (d, J=1.9 Hz, 2H), 3.93 (s, 3H), 3.90 (d, J=12.3 Hz, 1H), 3.65 (d, J=12.3 Hz, 1H), 2.48 (s, 3H).

### Example #99: 2-(14-methoxy-12-methyl-8-oxo-5,6,9,13-tetrazatricyclo[8.4.0.0^{2,6}]tetradeca-1(14),2,4,10,12-pentaen-9-yl)-N-[4-(trifluoromethyl)phenyl]acetamide

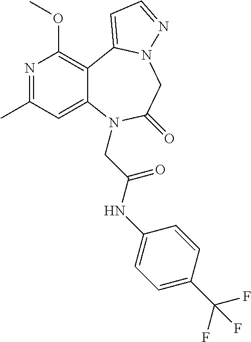 [SMILES: COc1nc(C)cc2c1-c1ccnn1CC(=O)N2CC(=O)Nc1ccc(C(F)(F)F)cc1]

The title product was prepared according to general procedure 1, starting from Intermediate N27 and 2-chloro-N-(4-trifluoromethyl-phenyl)acetamide. Purification by preparative HPLC (with acidic elution) afforded the title compound (70 mg, yield: 64%) as a white solid. LC-MS (Method B5) m/z [M+H]⁺: 446.2; rt: 2.20 min; purity: 99%. ¹H NMR (400 MHz, DMSO-d₆) δ 10.63 (s, 1H), 7.80 (d, J=8.5 Hz, 2H), 7.69 (d, J=8.5 Hz, 2H), 7.59 (d, J=2.0 Hz, 1H), 7.02 (s, 1H), 6.71 (d, J=2.0 Hz, 1H), 4.90 (s, 2H), 4.56 (d, J=7.5 Hz, 2H), 3.95 (s, 3H), 2.46 (s, 3H).

### Example #100: 2-(14-chloro-12-methyl-8-oxo-5,6,9,13-tetrazatricyclo[8.4.0.0^{2,6}]tetradeca-1(14),2,4,10,12-pentaen-9-yl)-N-[4-(trifluoromethyl)phenyl]acetamide

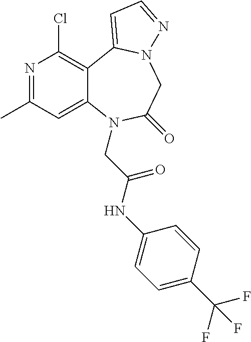 [SMILES: Cc1cc2c(c(Cl)n1)-c1ccnn1CC(=O)N2CC(=O)Nc1ccc(C(F)(F)F)cc1]

### Step 1: Synthesis of 2-(14-hydroxy-12-methyl-8-oxo-5,6,9,13-tetrazatricyclo[8.4.0.0^{2,6}]tetradeca-1(14),2,4,10,12-pentaen-9-yl)-N-[4-(trifluoromethyl)phenyl]acetamide #100_1

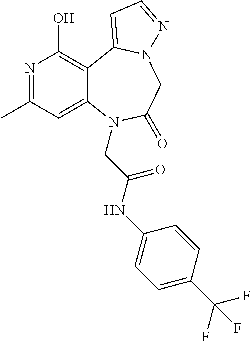 [SMILES: Cc1cc2c(c(O)n1)-c1ccnn1CC(=O)N2CC(=O)Nc1ccc(C(F)(F)F)cc1]

Example #99 (245 mg, 0.539 mmol) and KI (268 mg, 1.62 mmol) were suspended in dry MeCN (30 mL) and TMSCl (0.205 mL, 1.62 mmol) was then added. The reaction mixture was stirred at 80° C. for 4 h and then concentrated under vacuum. The residue was partitioned between EtOAc (50 mL) and water (30 mL). The layers were separated and the organic phase was washed with saturated sodium thiosulphate solution (10 mL). The organic phase was filtered through a hydrophobic frit and concentrated under vacuum. The residue was triturated in MTBE (20 mL) and the solid was collected, washed with MTBE (2×10 mL) and dried to afford the title intermediate #100_1 (238 mg, quantitative yield) as a beige solid. LC-MS (Method B5) m/z [M+H]⁺: 432.2, rt: 1.71 min, purity: 99%. ¹H NMR (400 MHz, DMSO-d₆) δ 12.01 (s, 1H), 10.73 (s, 1H), 7.81 (d, J=8.4 Hz, 2H), 7.69 (d, J=8.6 Hz, 2H), 7.54 (d, J=2.0 Hz, 1H), 6.76 (d, J=2.0 Hz, 1H), 6.20 (s, 1H), 4.93-4.82 (m, 2H), 4.54-4.44 (m, 2H), 2.23 (s, 3H).

### Step 2: Synthesis of 2-(14-chloro-12-methyl-8-oxo-5,6,9,13-tetrazatricyclo[8.4.0.0^{2,6}]tetradeca-1(14),2,4,10,12-pentaen-9-yl)-N-[4-(trifluoromethyl)phenyl]acetamide #100

2-(14-hydroxy-12-methyl-8-oxo-5,6,9,13-tetrazatricyclo[8.4.0.0^{2,6}]tetradeca-1(14),2,4,10,12-pentaen-9-yl)-N-[4-(trifluoromethyl)phenyl]acetamide #100_1 (25 mg, 0.0580 mmol) was dissolved in POCl₃ (5 mL) and stirred at 100° C. for 3 h. The reaction mixture was cooled to RT and slowly poured into water (50 mL). After 1 h, to the resulting solution was added solid Na₂CO₃ (~1 g) portion wise to reach pH~7-8. The aqueous layer was extracted with EtOAc (2×25 mL) and the combined organic extracts were filtered through a hydrophobic frit and concentrated under vacuum. The residue was purified by flash chromatography on silica gel (using a gradient of 0 to 50% MeCN in DCM as eluent) to afford the title compound as a white solid (5 mg, yield: 19%). LC-MS (Method A7) m/z [M+H]⁺: 450.1/452.1, rt: 2.12 min, purity: 99%. ¹H NMR (400 MHz, DMSO-d₆) δ 10.64 (s, 1H), 7.79 (d, J=8.5 Hz, 2H), 7.73-7.63 (m, 3H), 7.46 (s, 1H), 6.90 (d, J=2.1 Hz, 1H), 5.10-4.89 (m, 2H), 4.65-4.55 (m, 2H), 2.53 (s, 3H).

### Example #101: 2-(3,10-difluoro-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

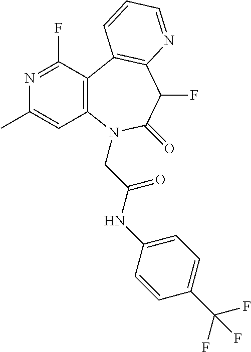 [SMILES: Cc1cc2c(c(F)n1)-c1cccnc1C(F)C(=O)N2CC(=O)Nc1ccc(C(F)(F)F)cc1]

The title product was prepared according to general procedure 1, starting from Intermediate N28 and 2-chloro-N-(4-trifluoromethyl-phenyl)acetamide. Purification by preparative HPLC (with basic elution) afforded the title compound (6 mg, yield: 8%) as a white solid. LC-MS (Method B3) m/z [M+H]⁺: 463.0; rt: 4.17 min; purity: 96%. LC-MS (Method A3) m/z [M+H]⁺: 463.0; rt: 4.73 min; purity: 95%. ¹H NMR (400 MHz, DMSO-d₆) δ 10.64 (s, 1H), 8.73 (dd, J=4.8, 1.7 Hz, 1H), 8.20 (dd, J=7.9, 1.7 Hz, 1H), 7.76 (d, J=8.8 Hz, 2H), 7.68 (d, J=8.8 Hz, 2H), 7.61 (dd, J=7.9, 4.8 Hz, 1H), 7.40 (s, 1H), 6.29 (d, J=45.3 Hz, 1H), 4.67 (d, J=16.9 Hz, 1H), 4.64 (d, J=16.9 Hz, 1H) 2.49 (s, 3H).

### Example #102: 2-(3-fluoro-5-methyl-9,12-dioxo-4,8,11-triazatricyclo[9.4.0.0^{2,7}]pentadeca-1(15),2(7),3,5,13-pentaen-8-yl)-N-[4-(trifluoromethyl)phenyl]acetamide

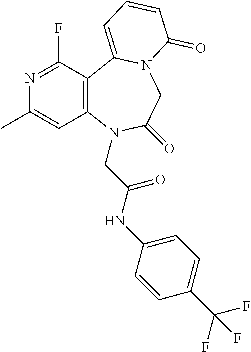 [SMILES: Cc1cc2c(c(F)n1)-c1cccc(=O)n1CC(=O)N2CC(=O)Nc1ccc(C(F)(F)F)cc1]

The title product was prepared according to general procedure 1, starting from Intermediate N29 and 2-chloro-N-(4-trifluoromethyl-phenyl)acetamide. Purification by SFC (using 75% CO₂ and 25% CH₃CN as eluent) afforded the title compound (6 mg, yield: 7%) as a white solid. LC-MS (Method B1) m/z [M+H]⁺: 461.0; rt: 3.71 min; purity: 89%. LC-MS (Method A1) m/z [M+H]⁺: 461.0; rt: 3.99 min; purity: 97%. ¹H NMR (400 MHz, DMSO-d₆) δ 10.66 (s, 1H), 7.77 (d, J=8.5 Hz, 2H), 7.68 (d, J=8.5 Hz, 2H), 7.55 (dd, J=9.2, 7.1 Hz, 1H), 7.34 (s, 1H), 6.64-6.48 (m, 2H), 5.57 (d, J=13.8 Hz, 1H), 4.67 (s, 2H), 4.38 (d, J=13.8 Hz, 1H), 2.49 (s, 3H).

### Example #103: 2-(3-amino-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

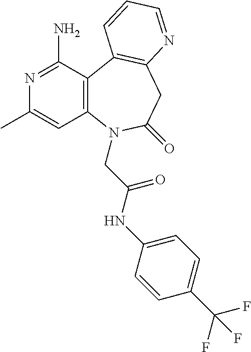 [SMILES: Cc1cc2c(c(N)n1)-c1cccnc1CC(=O)N2CC(=O)Nc1ccc(C(F)(F)F)cc1]

### Step 1: Synthesis of 2-(3-azido-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide #103_1

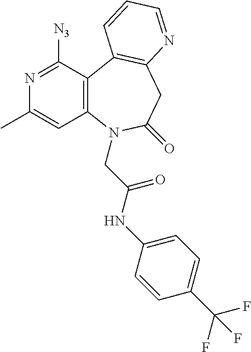 [SMILES: Cc1cc2c(c(N=[N+]=[N-])n1)-c1cccnc1CC(=O)N2CC(=O)Nc1ccc(C(F)(F)F)cc1]

To a solution of example #97 (35 mg, 0.073 mmol) in dry DMSO (0.8 mL) was added sodium azide (47 mg, 0.728 mmol) and acetic acid (40 μL, 0.728 mmol) and the reaction mixture was heated at 100° C. overnight. After cooling to room temperature, a saturated aqueous solution of NaHCO₃ (15 mL) was added and the mixture was extracted with EtOAc (3×20 mL). The organic layers were combined, washed with brine (40 mL), dried over Na₂SO₄, filtered and concentrated under vacuum to give the title compound (35 mg, yield: 90%) as a beige solid. LC-MS (Method A7) m/z: [M+H]⁺: 468; rt: 1.96 min; purity: 87%. ¹H NMR (400 MHz, DMSO-d₆) δ 10.67 (s, 1H), 8.72 (dd, J=4.8, 1.6 Hz, 1H), 8.59 (dd, J=8.0, 1.6 Hz, 1H), 7.80 (d, J=8.5 Hz, 2H), 7.69 (d, J=8.4 Hz, 2H), 7.66 (dd, J=8.0, 4.8 Hz, 1H), 7.53 (d, J=1.2 Hz, 1H), 4.70 (d, J=16.9 Hz, 1H), 4.64 (d, J=17.0 Hz, 1H), 3.92 (d, J=12.5 Hz, 1H), 3.85 (d, J=12.5 Hz, 1H), 2.93 (s, 3H).

### Step 2: Synthesis of 2-(3-amino-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide #103

A solution of 2-(3-azido-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide (35 mg, 0.066 mmol) and tri-n-butylphosphine (80 μL, 0.32 mmol) in 1,4-dioxane (1.8 mL), MeOH (1.2 mL) and water (1.2 mL) was heated at 80° C. overnight. Water (10 mL) and brine (10 mL) were added and the mixture was extracted with EtOAc (3×15 mL). The combined organic extracts were washed with brine (30 mL), dried over Na₂SO₄, filtered and concentrated under vacuum. The residue was purified by flash chromatography on silica gel (using a gradient of 0 to 10% MeOH in DCM as eluent) and trituration in MeOH to give the title compound (9 mg, yield: 30%) as a white solid. LC-MS (Method A7) m/z: [M+H]⁺: 442; rt: 1.38 min; purity: 97%. ¹H NMR (400 MHz, DMSO-d₆) δ 10.52 (s, 1H), 8.50 (dd, J=4.9, 1.6 Hz, 1H), 8.17 (dd, J=7.9, 1.7 Hz, 1H), 7.78 (d, J=8.5 Hz, 2H), 7.68 (d, J=8.6 Hz, 2H), 7.42 (dd, J=7.9, 4.8 Hz, 1H), 6.56 (s, 1H), 5.93 (s, 2H), 4.46 (d, J=16.8 Hz, 1H), 4.40 (d, J=16.8 Hz, 1H), 3.73 (d, J=12.1 Hz, 1H), 3.65 (d, J=12.1 Hz, 1H), 2.30 (s, 3H).

### Example #104: 2-(14-methoxy-4,12-dimethyl-8-oxo-5,6,9,13-tetrazatricyclo[8.4.0.0^{2,6}]tetradeca-1(14),2,4,10,12-pentaen-9-yl)-N-[4-(trifluoromethyl)phenyl]acetamide

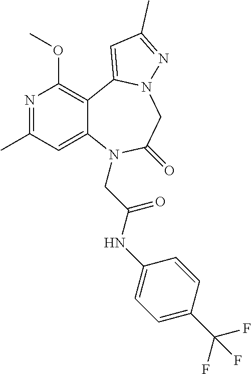 [SMILES: COc1nc(C)cc2c1-c1cc(C)nn1CC(=O)N2CC(=O)Nc1ccc(C(F)(F)F)cc1]

The title product was prepared according to general procedure 1 starting from Intermediate N30 and 2-chloro-N-(4-trifluoromethyl-phenyl)acetamide. Purification by flash chromatography on silica gel (using a gradient of 0-50% MeCN/DCM as eluent) and trituration in a 1:1 mixture of MTBE/iso-hexane (5 mL) afforded the title compound (22.0 mg, yield: 38%) as a white solid. LC-MS (Method A7): m/z [M+H]⁺: 460.2; rt: 2.32 min; purity: 99%. ¹H NMR (400 MHz, DMSO-d₆) δ 10.62 (s, 1H), 7.80 (d, J=8.5 Hz, 2H), 7.69 (d, J=8.5 Hz, 2H), 7.00 (s, 1H), 6.49 (s, 1H), 4.79 (s, 2H), 4.55 (d, J=9.6 Hz, 2H), 3.94 (s, 3H), 2.45 (s, 3H), 2.23 (s, 3H).

### Example #105: 2-(14-chloro-4,12-dimethyl-8-oxo-5,6,9,13-tetrazatricyclo[8.4.0.0^{2,6}]tetradeca-1(14),2,4,10,12-pentaen-9-yl)-N-[4-(trifluoromethyl)phenyl]acetamide

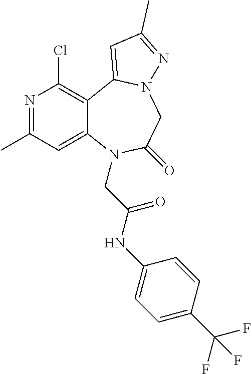 [SMILES: Cc1cc2c(c(Cl)n1)-c1cc(C)nn1CC(=O)N2CC(=O)Nc1ccc(C(F)(F)F)cc1]

The title product was prepared according to general procedure 1, starting from intermediate N30 and 2-chloro-N-(4-trifluoromethyl-phenyl)acetamide. Purification by flash chromatography on silica gel (using a gradient of 0-50% MeCN/DCM as eluent) afforded the title compound (38 mg, yield: 58%) as a white solid. LC-MS (Method A7): m/z [M+H]⁺: 464.1/466.2; rt: 2.21 min; purity: 98%. ¹H NMR (400 MHz, DMSO-d₆) δ 10.64 (s, 1H), 7.79 (d, J=8.5 Hz, 2H), 7.70 (d, J=8.5 Hz, 2H), 7.44 (s, 1H), 6.68 (s, 1H), 5.01-4.76 (m, 2H), 4.58 (d, J=2.5 Hz, 2H), 2.52 (s, 3H), 2.25 (s, 3H).

### Example #106: 2-(14-fluoro-4,12-dimethyl-8-oxo-5,6,9,13-tetrazatricyclo[8.4.0.0^{2,6}]tetradeca-1(10),2,4,11,13-pentaen-9-yl)-N-[4-(trifluoromethyl)phenyl]acetamide

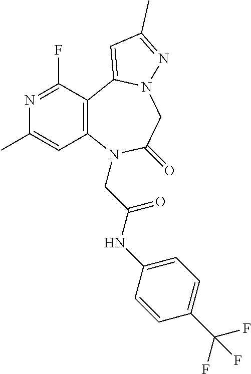 [SMILES: Cc1cc2c(c(F)n1)-c1cc(C)nn1CC(=O)N2CC(=O)Nc1ccc(C(F)(F)F)cc1]

The title product was prepared according to general procedure 1 starting from Intermediate N107 and 2-chloro-N-(4-trifluoromethylphenyl)acetamide (CAS 2707-23-5). LC-MS (Method B4) m/z [M+H]⁺: 448.5; rt: 3.98 min; purity: 91%. LC-MS (Method A4) m/z [M−H]⁻: 446.4; rt: 4.43 min; purity: 90%.

### Example #107: 2-(1-chloro-7-methoxy-3-methyl-6-oxo-7H-pyrido[3,4-a][3]benzazepin-5-yl)-N-[4-(trifluoromethyl)phenyl]acetamide

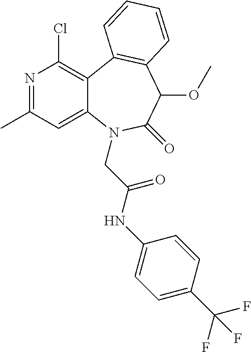 [SMILES: COC1C(=O)N(CC(=O)Nc2ccc(C(F)(F)F)cc2)c2cc(C)nc(Cl)c2-c2ccccc21]

The title product was prepared according to general procedure 1, starting from Intermediate N32 and 2-chloro-N-[4-(trifluoromethyl)phenyl]acetamide (CAS 2707-23-5). Purification by flash chromatography on silica gel (using a gradient of 0-50% EtOAc/iso-hexane as eluent) afforded the title compound (107 mg, yield: 72%) as a white solid. LC-MS (Method A7): m/z [M+H]⁺: 490.2/492.1: rt: 2.38 min; purity: 99%. ¹H NMR (400 MHz, DMSO-d₆) δ 10.61 (s, 1H), 7.80-7.78 (m, 3H), 7.70 (d, J=8.8 Hz, 2H), 7.57-7.50 (m, 2H), 7.46 (td, J=7.2, 2.1 Hz, 1H), 7.39 (s, 1H), 4.83 (s, 1H), 4.56 (d, J=1.6 Hz, 2H), 3.43 (s, 3H), 2.53 (s, 3H).

### Example #108: 2-(3,5-dimethoxy-9-oxo-4,8,12-triazatricyclo[9.4.0.0^{2,7}]pentadeca-1(11),2,4,6,12,14-hexaen-8-yl)-N-[4-(trifluoromethyl)phenyl]acetamide

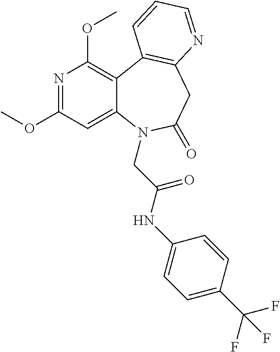 [SMILES: COc1cc2c(c(OC)n1)-c1cccnc1CC(=O)N2CC(=O)Nc1ccc(C(F)(F)F)cc1]

The title product was prepared according to general procedure 1, starting from Intermediate N33 and 2-chloro-N-(4-trifluoromethyl-phenyl)acetamide. Purification by flash chromatography on silica gel (using a gradient of 0-5% MeOH/DCM as eluent) and trituration with MTBE gave the title compound (76 mg, yield: 72%) as a white solid. LC-MS (Method B5): m/z [M+H]⁺: 473.2; rt: 2.24 min; purity: 98%. ¹H NMR (400 MHz, DMSO-d₆) δ 10.57 (s, 1H), 8.53 (dd, J=4.8, 1.7 Hz, 1H), 8.17 (dd, J=8.0, 1.7 Hz, 1H), 7.78 (d, J=8.5 Hz, 2H), 7.68 (d, J=8.5 Hz, 2H), 7.45 (dd, J=8.0, 4.8 Hz, 1H), 7.03 (s, 1H), 4.53 (s, 2H), 3.90 (s, 3H), 3.74 (d, J=12.3 Hz, 1H), 3.68 (d, J=12.3 Hz, 1H), 2.46 (s, 3H).

### Example #109: 2-(4-methoxy-5,10-dimethyl-9-oxo-3,8,12-triazatricyclo[9.4.0.0^{2,7}]pentadeca-1(11),2,4,6,12,14-hexaen-8-yl)-N-[4-(trifluoromethyl)phenyl]acetamide

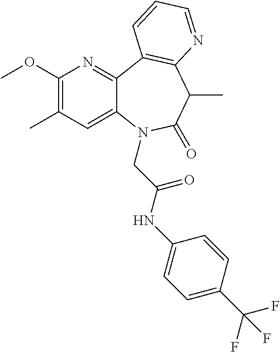 [SMILES: COc1nc2c(cc1C)N(CC(=O)Nc1ccc(C(F)(F)F)cc1)C(=O)C(C)c1ncccc1-2]

The title product was prepared according to general procedure 1, starting from 4-methoxy-5,10-dimethyl-3,8,12-triazatricyclo[9.4.0.0^{2,7}]pentadeca-1(11),2(7),3,5,12,14-hexaen-9-one N68_4 and 2-chloro-N-(4-trifluromethyl-phenyl)acetamide. Purification by flash chromatography on silica gel (using a gradient of 0-50% MeCN/DCM as eluent) afforded the title compound (22 mg, yield: 63%) as a beige solid. LC-MS (Method A7): m/z [M+H]⁺: 471.2; rt: 2.21 min; purity: 97%. ¹H NMR (400 MHz, DMSO-d₆) δ 10.54 (s, 1H), 8.69 (dd, J=4.8, 1.8 Hz, 1H), 8.36 (dd, J=7.8, 1.8 Hz, 1H), 7.81-7.73 (m, 3H), 7.67 (d, J=8.6 Hz, 2H), 7.54 (dd, J=7.8, 4.7 Hz, 1H), 4.49 (s, 2H), 4.00 (s, 3H), 3.63 (d, J=6.7 Hz, 1H), 2.25 (s, 3H), 1.51 (d, J=6.7 Hz, 3H).

### Example #110: 2-(3-fluoro-5-methyl-9-oxo-4,8,12,13-tetrazatricyclo[9.4.0.0^{2,7}]pentadeca-1(11),2,4,6,12,14-hexaen-8-yl)-N-[4-(trifluoromethyl)phenyl]acetamide

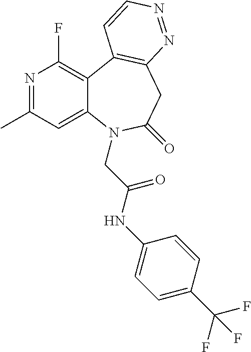 [SMILES: Cc1cc2c(c(F)n1)-c1ccnnc1CC(=O)N2CC(=O)Nc1ccc(C(F)(F)F)cc1]

The title product was prepared according to general procedure 1, starting from Intermediate N34 and 2-chloro-N-(4-trifluoromethyl-phenyl)acetamide. Purification by flash chromatography on silica gel (using a gradient of 0-100% MeCN/DCM, followed by 10% MeOH/DCM as eluent) afforded the title compound as an off-white solid (16 mg, yield: 40%). LC-MS (Method A7): m/z [M+H]⁺: 446.2; rt: 1.92 min, purity: 96%. ¹H NMR (400 MHz, DMSO-d₆) δ 10.63 (s, 1H), 9.37 (d, J=5.3 Hz, 1H), 8.07 (t, J=4.9 Hz, 1H), 7.77 (d, J=8.6 Hz, 2H), 7.68 (d, J=8.6 Hz, 2H), 7.44 (s, 1H), 4.63 (d, J=1.5 Hz, 2H), 4.20-4.02 (m, 2H), 2.52 (s, 3H).

### Example #111: 2-(14-fluoro-7,12-dimethyl-8-oxo-5,6,9,13-tetrazatricyclo[8.4.0.0^{2,6}]tetradeca-1(10),2,4,11,13-pentaen-9-yl)-N-[4-(trifluoromethyl)phenyl]acetamide

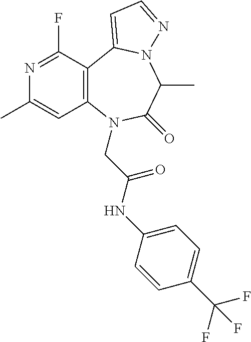 [SMILES: Cc1cc2c(c(F)n1)-c1ccnn1C(C)C(=O)N2CC(=O)Nc1ccc(C(F)(F)F)cc1]

The title product was prepared according to procedure 1, starting from Intermediate N35 and 2-chloro-N-(4-trifluoromethylphenyl)acetamide (CAS 2707-23-5). Purification by preparative HPLC (with basic elution) afforded the title compound as a white solid (4 mg, yield: 11%). LC-MS (Method B1) m/z [M+H]⁺: 448.1; rt: 2.64 min; purity: 98%. LC-MS (Method A1) m/z [M+H]⁺: 448.1; rt: 2.66 min; purity>99%.

### Example #112: 2-(15-fluoro-8,13-dimethyl-9-oxo-4,6,10,14-tetrazatricyclo[9.4.0.0^{2,7}]pentadeca-1(15),2(7),3,5,11,13-hexaen-10-yl)-N-[4-(trifluoromethyl)phenyl]acetamide

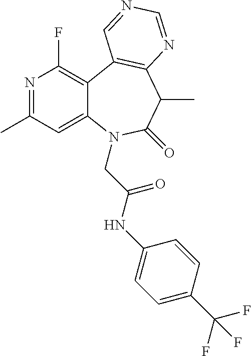 [SMILES: Cc1cc2c(c(F)n1)-c1cncnc1C(C)C(=O)N2CC(=O)Nc1ccc(C(F)(F)F)cc1]

The title product was prepared according to general procedure 1, starting from Intermediate N36 and 2-chloro-N-(4-trifluoromethyl-phenyl)acetamide. Purification by flash chromatography on silica gel (using a gradient of 0-5% MeOH/DCM as eluent) and flash reverse phase chromatography on C18 silica gel (Neutral, using a gradient of 20-100% MeCN/H₂O as eluent) afforded the title compound (27 mg, yield: 56%) as a white solid. LC-MS (Method A7): m/z [M+H]⁺: 460.2; rt 2.13: min; purity: 98%. ¹H NMR (400 MHz, DMSO-d₆) δ 10.65 (s, 1H), 9.27 (s, 1H), 9.15 (d, J=4.6 Hz, 1H), 7.77 (d, J=8.5 Hz, 2H), 7.68 (d, J=8.5 Hz, 2H), 7.43 (s, 1H), 4.63 (s, 2H), 3.93 (q, J=6.6 Hz, 1H), 2.53 (s, 3H), 1.49 (d, J=6.6 Hz, 3H).

### Example #113: 2-(5-fluoro-3-methoxy-9-oxo-4,8,12-triazatricyclo[9.4.0.0^{2,7}]pentadeca-1(11),2,4,6,12,14-hexaen-8-yl)-N-[4-(trifluoromethyl)phenyl]acetamide

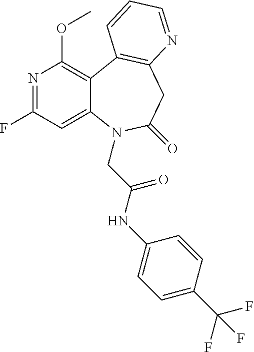 [SMILES: COc1nc(F)cc2c1-c1cccnc1CC(=O)N2CC(=O)Nc1ccc(C(F)(F)F)cc1]

The title product was prepared according to general procedure 1, starting from Intermediate N37 and 2-chloro-N-(4-trifluoromethyl-phenyl)acetamide. Purification by flash chromatography on silica gel (using a gradient of 0-5% MeOH/DCM as eluent) and reverse phase preparative HPLC (BEH column C18, using a gradient of 0.1% NH₃ in H₂O/MeCN as eluent) gave the title compound (30 mg, yield: 53%) as a white solid. LC-MS (Method B5): m/z [M+H]⁺: 461.2; rt: 2.22 min; purity>99%. ¹H NMR (400 MHz, DMSO-d₆) δ 10.61 (s, 1H), 8.56 (dd, J=4.8, 1.7 Hz, 1H), 8.20 (dd, J=7.9, 1.7 Hz, 1H), 7.77 (d, J=8.5 Hz, 2H), 7.68 (d, J=8.5 Hz, 2H), 7.47 (dd, J=8.0, 4.8 Hz, 1H), 6.93 (s, 1H), 4.63 (d, J=16.9 Hz, 1H), 4.56 (d, J=16.9 Hz, 1H), 3.92 (s, 3H), 3.85 (d, J=12.4 Hz, 1H), 3.70 (d, J=12.4 Hz, 1H).

### Example #114: 2-(3-chloro-5,10-dimethyl-9-oxo-4,8,12-triazatricyclo[9.4.0.0^{2,7}]pentadeca-1(11),2,4,6,12,14-hexaen-8-yl)-N-[4-(trifluoromethyl)phenyl]acetamide

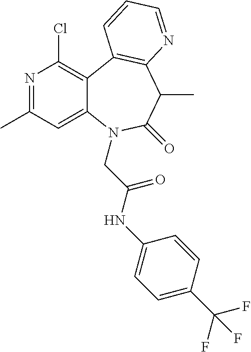 [SMILES: Cc1cc2c(c(Cl)n1)-c1cccnc1C(C)C(=O)N2CC(=O)Nc1ccc(C(F)(F)F)cc1]

The title product was prepared according to general procedure 1, starting from Intermediate N31 and 2-chloro-N-[4-(trifluoromethyl)phenyl]acetamide (CAS 2707-23-5). Purification by column chromatography on silica gel (using a gradient of 0-6% MeOH/DCM as eluent) followed by reverse phase chromatography (using a gradient of 0.1% Formic Acid in H₂O/MeCN as eluent) afforded the title compound as a white solid (139 mg, yield: 45%). LC-MS (Method A7): m/z [M+H]⁺: 475.1/477.1; rt: 2.25 min; purity: 99%. ¹H NMR (400 MHz, DMSO-d₆) δ 10.59 (s, 1H), 8.66 (dd, J=4.8, 1.7 Hz, 1H), 8.24 (dd, J=7.9, 1.7 Hz, 1H), 7.85-7.60 (m, 4H), 7.55-7.39 (m, 2H), 4.72-4.38 (m, 2H), 3.82 (q, J=6.6 Hz, 1H), 2.54 (s, 3H), 1.49 (d, J=6.6 Hz, 3H).

The following Examples 115-117 have been prepared according to general procedure 1, starting from Intermediate N31 with 2-chloro-N-(4-cyanophenyl)acetamide (CAS 114807-80-6), 2-chloro-N-[4-(difluoromethyl)phenyl]acetamide (CAS 872533-93-2) and 2-chloro-N-(4-chloro-3-fluoro-phenyl)acetamide (CAS 895641-02-8), respectively. Example #116 was obtained by separation of the racemate by chiral chromatography (SFC-Chiralpak OD-I, CO₂+EtOH 20%). Example #117 was obtained by separation of the racemate by chiral chromatography (SFC-Chiralpak IC, CO₂+MeOH 20%).

| Ex | Structure Example | Example Name | Protocolumn and characterization |
|---|---|---|---|
| #115 | 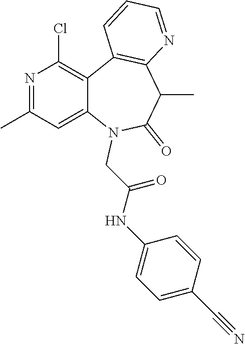 [SMILES: Cc1cc2c(c(Cl)n1)-c1cccnc1C(C)C(=O)N2CC(=O)Nc1ccc(C#N)cc1] | 2-(3-chloro-5,10-dimethyl- 9-oxo-4,8,12-triazatricyclo [9.4.0.0^{2,7}]pentadeca-1(11), 2,4,6,12,14-hexaen-8-yl)- N-(4-cyanophenyl)acet- amide | LC-MS (Method A7) m/z [M + H]⁺: 432/434; rt: 1.90 min; purity: 99%. ¹H NMR (400 MHz, DMSO-d₆) δ 10.66 (s, 1H), 8.69-8.59 (m, 1H), 8.27-8.16 (m, 1H), 7.83-7.69 (m, 4H), 7.54-7.41 (m, 2H), 4.64-4.49 (m, 2H), 3.84-3.76 (m, 1H), 2.53 (s, 3H), 1.53-1.43 (m, 3H). |
| #116 | 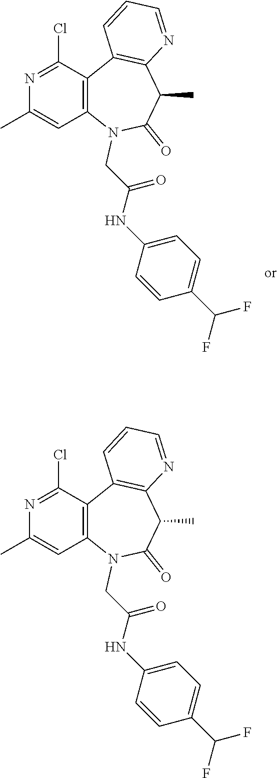 [SMILES: Cc1cc2c(c(Cl)n1)-c1cccnc1[C@@H](C)C(=O)N2CC(=O)Nc1ccc(C(F)F)cc1 ; Cc1cc2c(c(Cl)n1)-c1cccnc1[C@H](C)C(=O)N2CC(=O)Nc1ccc(C(F)F)cc1] | Enantiomer (10R) or (10S) of 2-[3-chloro-5,10- dimethyl-9-oxo-4,8,12- triazatricyclo[9.4.0.0^{2,7}] pentadeca-1(11),2(7),3,5, 12,14-hexaen-8-yl]-N-[4- (difluoromethyl)phenyl] acetamide | LCMS (Method A7): m/z [M + H]⁺: 457.1/459.2; rt: 2.05 min; purity: >99%. ¹H NMR (400 MHz, DMSO- d₆) δ 10.44 (s, 1H), 8.65 (dd, J = 4.8, 1.7 Hz, 1H), 8.23 (dd, J = 7.9, 1.7 Hz, 1H), 7.68 (d, J = 8.4 Hz, 2H), 7.54-7.46 (m, 3H), 7.45 (s, 1H), 6.96 (t, J = 56.1 Hz, 1H), 4.59 (d, J = 16.9 Hz, 1H), 4.52 (d, J = 16.9 Hz, 1H), 3.80 (q, J = 6.5 Hz, 1H), 2.53 (s, 3H), 1.48 (d, J = 6.5 Hz, 3H). Chiral purity: 99%; rt = 2.14 min (second eluting enantio- mer). For information, first eluting enantiomer rt = 1.56 min. Both measured by HPLC, Chiralpak IB from Daicel, MeOH 100 % DEA 0.1 %) |
| #117 | 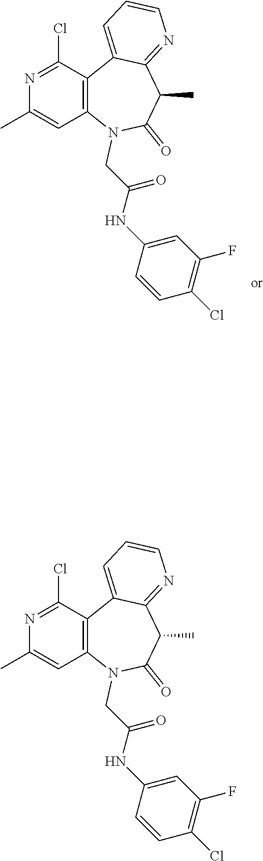 [SMILES: Cc1cc2c(c(Cl)n1)-c1cccnc1[C@@H](C)C(=O)N2CC(=O)Nc1ccc(Cl)c(F)c1 ; Cc1cc2c(c(Cl)n1)-c1cccnc1[C@H](C)C(=O)N2CC(=O)Nc1ccc(Cl)c(F)c1] | Enantiomer (10R) or (10S) of 2-[3-chloro-5,10-dimeth- yl-9-oxo-4,8,12-triazatri- cyclo[9.4.0.0^{2,7}]pentadeca- 1(15),2(7),3,5,11,13- hexaen-8-yl]-N-(4-chloro- 3-fluorophenyl)acetamide | LCMS (Method A7): m/z [M + H]⁺ 459.1/461.1; rt: 2.20 min; purity: >99%. ¹H NMR (400 MHz, DMSO- d₆) δ 10.55 (s, 1H), 8.65 (dd, J = 4.8, 1.7 Hz, 1H), 8.23 (dd, J = 7.9, 1.7 Hz, 1H), 7.72 (dd, J = 11.9, 2.4 Hz, 1H), 7.55-7.46 (m, 2H), 7.44 (s, 1H), 7.30 (ddd, J = 8.8, 2.5, 1.0 Hz, 1H), 4.57 (d, J = 16.8 Hz, 1H), 4.49 (d, J = 16.8 Hz, 1H), 3.80 (q, J = 6.5 Hz, 1H), 2.53 (s, 3H), 1.47 (d, J = 6.5 Hz, 3H). Chiral purity: >99%; rt = 1.86 min (second eluting enantiomer). For information, first eluting enantiomer rt = 1.51 min. Both measured by HPLC, Chiralpak IB from Daicel, ACN 100%-DEA 0.1 %): |

### Example #118: 2-[2-[acetyl(methyl)amino]-5-oxo-6H-thiazolo[4,5-d][3]benzazepin-4-yl]-N-[4-(trifluoromethyl)phenyl]acetamide

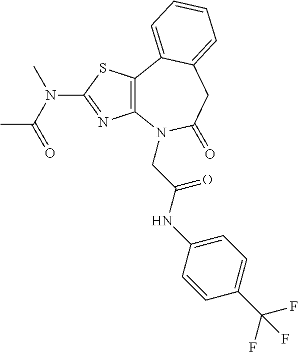 [SMILES: CC(=O)N(C)c1nc2c(s1)-c1ccccc1CC(=O)N2CC(=O)Nc1ccc(C(F)(F)F)cc1]

A suspension of 2-[2-(methylamino)-5-oxo-6H-thiazolo[4,5-d][3]benzazepin-4-yl]-N-[4-(trifluoromethyl)phenyl]acetamide (Intermediate N66, 80 mg, 0.16 mmol) in acetic anhydride (1.6 mL, 17 mmol) was heated at 70° C. for 6 h. The reaction was stopped and was kept at room temperature for 16 h. The reaction mixture was neutralized by addition of saturated NaHCO₃ and extracted with EtOAc (3×20 mL). The combined organic layers were washed with brine (30 mL), dried over MgSO₄, filtered off and concentrated under vacuum. The resulting yellow solid was triturated with Et₂O to give the title product (60 mg, yield: 75%) as of an off-white solid. LC-MS (Method-A1) m/z: [M+H]⁺: 489.0, rt: 1.46 min, purity: 99%. ¹H NMR (400 MHz, DMSO-d₆) δ 10.52 (s, 1H), 7.75 (d, J=8.5 Hz, 2H), 7.65 (d, J=8.5 Hz, 2H), 7.53-7.37 (m, 4H), 4.72 (s, 2H), 3.66 (s, 2H), 3.56 (s, 3H), 2.39 (s, 3H). LC-MS (Method B2) m/z [M+H]⁺: 489.4; rt: 4.60 min; purity: 98%. LC-MS (Method A2) m/z [M+H]⁺: 489.4; rt: 4.75 min; purity: 98%. ¹H NMR (400 MHz, DMSO-d₆) δ 10.52 (s, 1H), 7.75 (d, J=8.6 Hz, 2H), 7.65 (d, J=8.6 Hz, 2H), 7.56-7.34 (m, 4H), 4.72 (s, 2H), 3.66 (s, 2H), 3.56 (s, 3H), 2.39 (s, 3H).

### Example #119: N-[4-(trifluoromethyl)phenyl]-2-(3,5,10-trimethyl-4,9-dioxo-3,8,12-triazatricyclo[9.4.0.0^{2,7}]pentadeca-1(11),2(7),5,12,14-pentaen-8-yl)acetamide

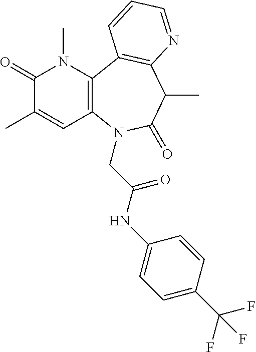 [SMILES: Cc1cc2c(n(C)c1=O)-c1cccnc1C(C)C(=O)N2CC(=O)Nc1ccc(C(F)(F)F)cc1]

[2-(3,5,10-Trimethyl-4,9-dioxo-3,8,12-triazatricyclo[9.4.0.0^{2,7}]pentadeca-1 (11),2(7),5,12,14-pentaen-8-yl)acetyl]oxylithium (Intermediate N68, 200 mg, 0.570 mmol) was dissolved in dry DMF (20 mL) then 4-(trifluoromethyl)aniline (110 mg, 0.684 mmol), N,N-diisopropylethylamine (0.296 mL, 1.71 mmol) and HATU (325 mg, 0.855 mmol) were sequentially added and the reaction mixture was stirred at room temperature for 18 hours. The reaction mixture was diluted with EtOAc (35 mL) and washed with water (2×20 mL) and brine (3×40 mL). The organic phase was dried over sodium sulfate, filtered and concentrated under vacuum. The residue was purified by flash chromatography on silica gel (using a gradient of 0 to 6% MeOH in DCM as eluent) to afford the title compound (106 mg, yield: 37%) as a light-yellow solid. ¹⁹F NMR (376 MHz, DMSO-d₆) δ−60.29. LC-MS (Method A7): m/z [M+H]⁺: 471.2; rt: 1.88 min; purity: 97%. ¹H NMR (400 MHz, DMSO-d₆) δ 10.45 (s, 1H), 8.70 (dd, J=4.7, 1.7 Hz, 1H), 8.20 (dd, J=8.0, 1.7 Hz, 1H), 7.73 (d, J=8.6 Hz, 2H), 7.69-7.61 (m, 3H), 7.49 (dd, J=8.0, 4.7 Hz, 1H), 4.43 (d, J=16.6 Hz, 1H), 4.26 (d, J=16.6 Hz, 1H), 3.92 (d, J=6.6 Hz, 1H), 3.37 (s, 3H), 2.12 (d, J=1.1 Hz, 3H), 1.50 (d, J=6.6 Hz, 3H).

### Example #120: N-(3-fluoro-4-methyl-phenyl)-2-(1,3,7-trimethyl-2,6-dioxo-7H-pyrido[3,2-d][3]benzazepin-5-yl)acetamide

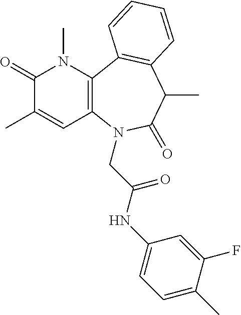 [SMILES: Cc1ccc(NC(=O)CN2C(=O)C(C)c3ccccc3-c3c2cc(C)c(=O)n3C)cc1F]

[2-(1,3,7-trimethyl-2,6-dioxo-7H-pyrido[3,2-d][3]benzazepin-5-yl)acetyl]oxylithium (Intermediate N69, 50.0 mg, 0.147 mmol) was dissolved in dry DMF (1.00 mL) and 3-fluoro-4-methyl-aniline (22.1 mg, 0.177 mmol), N,N-diisopropylethylamine (0.0765 mL, 0.441 mmol) and HATU (83.9 mg, 0.221 mmol) were sequentially added. The reaction mixture was stirred at RT for 18 hours. The reaction mixture was quenched with water (20 mL). The resulting solid was sonicated, filtered, washed with water (5 mL) and dried under vacuum at 40° C. The crude product was purified by Preparative HPLC (Waters, (0.1% Formic acid), Acidic, Waters X-Select Prep-C18, 5 μm, 19×50 mm column, 5-95% MeCN in Water) to afford the title compound (21.8 mg, 33%) as an off white solid. LCMS (Method A7): m/z [M+H]⁺: 434.2; rt: 2.01 min; purity: 71%. ¹H NMR (400 MHz, DMSO-d₆) δ 10.17 (s, 1H), 7.69 (dd, J=7.9, 1.3 Hz, 1H), 7.60-7.53 (m, 2H), 7.51-7.36 (m, 3H), 7.23-7.12 (m, 2H), 4.27-4.16 (m, 2H), 3.70 (q, J=6.7 Hz, 1H), 3.35 (s, 3H), 2.17 (d, J=1.8 Hz, 3H), 2.10 (d, J=1.1 Hz, 3H), 1.46 (d, J=6.7 Hz, 3H).

### Example #121: Enantiomer (7S) or (7R) of N-[4-(trifluoromethyl)phenyl]-2-[1,3,7-trimethyl-2,6-dioxo-7H-pyrido[3,2-d][3]benzazepin-5-yl]acetamide

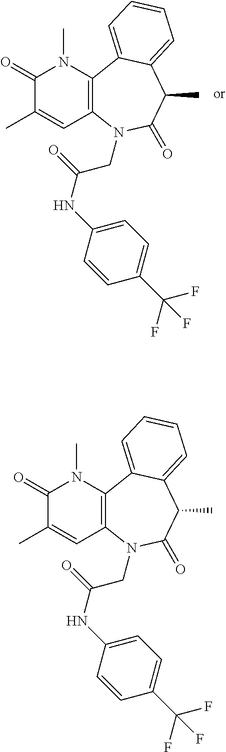 [SMILES: Cc1cc2c(n(C)c1=O)-c1ccccc1[C@@H](C)C(=O)N2CC(=O)Nc1ccc(C(F)(F)F)cc1 ; Cc1cc2c(n(C)c1=O)-c1ccccc1[C@H](C)C(=O)N2CC(=O)Nc1ccc(C(F)(F)F)cc1]

The title product was prepared according to a similar procedure as the one depicted for example #120 but starting from 4-(trifluoromethyl)aniline followed by chiral separation (SFC-Chiralpak IF, CO₂+MeOH 20%). LCMS (Method A7): m/z [M+H]⁺: 470.2; rt: 2.12 min; purity: >99%. ¹H NMR (400 MHz, DMSO-d₆) δ 10.43 (s, 1H), 7.75 (d, J=8.6 Hz, 2H), 7.71-7.65 (m, 3H), 7.60-7.53 (m, 2H), 7.44 (td, J=7.5, 1.2 Hz, 1H), 7.39 (d, J=7.9 Hz, 1H), 4.31 (d, J=16.6 Hz, 1H), 4.24 (d, J=16.6 Hz, 1H), 3.71 (q, J=6.7 Hz, 1H), 3.35 (s, 3H), 2.11 (d, J=1.2 Hz, 3H), 1.46 (d, J=6.7 Hz, 3H). Chiral purity: >99%; rt=3.27 min (second eluting enantiomer). For information, first eluting enantiomer rt=1.82 min. Both measured by HPLC, Chiralpak IF from Daicel, EtOH 50%—heptane 50%—DEA 0.1%)

### Example #122: 2-[1-chloro-7-(methoxymethyl)-3-methyl-6-oxo-7H-pyrido[3,4-a][3]benzazepin-5-yl]-N-[4-(trifluoromethyl)phenyl]acetamide

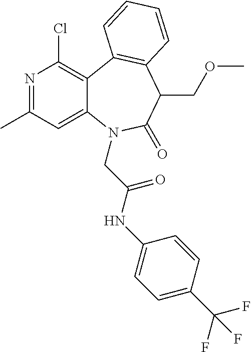 [SMILES: COCC1C(=O)N(CC(=O)Nc2ccc(C(F)(F)F)cc2)c2cc(C)nc(Cl)c2-c2ccccc21]

The title product was prepared according to general procedure 1, starting from Intermediate N39 and 2-chloro-N-[4-(trifluoromethyl)phenyl]acetamide (CAS 2707-23-5). Purification by flash chromatography on silica gel (using a gradient of 0-50% EtOAc/Iso-hexane as eluent) afforded the title compound (134 mg, yield: 73%) as a white solid. LC-MS (Method A7): m/z [M+H]⁺: 504.1/506.2; rt: 2.44 min; purity: 98% purity. ¹H NMR (400 MHz, DMSO-d₆) δ 10.57 (s, 1H), 7.81-7.74 (m, 3H), 7.68 (d, J=8.8 Hz, 2H), 7.51 (td, J=7.6, 1.5 Hz, 1H), 7.46-7.41 (m, 2H), 7.29 (d, J=7.7 Hz, 1H), 4.61-4.49 (m, 2H), 4.11-4.05 (m, 2H), 3.60 (t, J=7.0 Hz, 1H), 3.30 (s, 3H), 2.53 (s, 3H).

### Example #123: 2-(14-fluoro-12-methyl-8-oxo-4,5,9,13-tetrazatricyclo[8.4.0.0^{2,6}]tetradeca-1(10),2,5,11,13-pentaen-9-yl)-N-[4-(trifluoromethyl)phenyl]acetamide

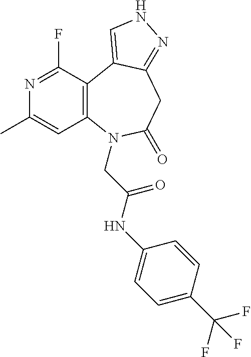 [SMILES: Cc1cc2c(c(F)n1)-c1c[nH]nc1CC(=O)N2CC(=O)Nc1ccc(C(F)(F)F)cc1]

### Step 1: Synthesis of ethyl 2-(1H-pyrazol-3-yl)acetate #123_1

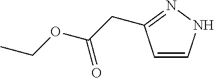

To a suspension of 2-(1H-pyrazol-3-yl)acetic acid (9.56 g, 42.5 mmol) in ethanol (150 mL) was added concentrated sulfuric acid (2.0 mL, 35.6 mmol) and the reaction mixture was stirred at reflux overnight. The reaction mixture was cooled to room temperature, filtered through Celite®, the filter cake was washed with EtOAc (50 mL) and the filtrate was evaporated to dryness. The residue was taken in saturated aqueous NaHCO₃ solution (300 mL) and EtOAc (150 mL). The layers were separated and the aqueous layer was extracted with EtOAc (2×100 mL). The combined organic layers were washed with saturated aqueous NaHCO₃ solution (50 mL), brine (2×50 mL) then dried over sodium sulfate, filtered and concentrated under vacuum to give the title compound (2.52 g, yield: 36%) as a brown oil. LC-MS (Method A7) m/z: [M+H]⁺: 155; rt: 1.01 min; purity: 95%. ¹H NMR (400 MHz, DMSO-d₆) δ 12.61 (br. s, 1H), 7.62 (br. s, 1H), 6.14 (d, J=2.1 Hz, 1H), 4.08 (q, J=7.1 Hz, 2H), 3.62 (s, 2H), 1.18 (t, J=7.1 Hz, 3H).

### Step 2: Synthesis of ethyl 2-(4-bromo-1H-pyrazol-3-yl)acetate #123_2

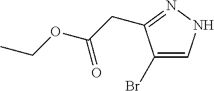

At 0° C., to a solution of ethyl 2-(1H-pyrazol-3-yl)acetate (#123_1, 2.52 g, 15.5 mmol) in dry DMF (20.0 mL) was added N-bromosuccinimide (3.02 g, 17.0 mmol) in three portions and the reaction mixture was stirred at 0° C. for 5 min then at room temperature for 1 h. The reaction mixture was neutralized with saturated aqueous Na₂S₂O₃ (200 mL) and extracted with EtOAc (3×50 mL). The combined organic extracts were washed with saturated aqueous Na₂S₂O₃ (50 mL), aqueous LiCl solution (20% w/w, 2×50 mL), brine (50 mL), dried over sodium sulfate, filtered and concentrated under vacuum. Purification by flash chromatography on silica gel (using a gradient of 0 to 100% Acetone in iso-hexane as eluent) gave the title compound (2.59 g, yield: 69%). LC-MS (Method A7) m/z: [M+H]⁺: 233/235; rt: 1.47 min; purity>99%,

### Step 3: Synthesis of ethyl 2-(4-bromo-1-tetrahydropyran-2-yl-pyrazol-3-yl)acetate #123_3

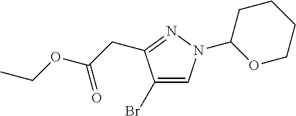 [SMILES: CCOC(=O)Cc1nn(C2CCCCO2)cc1Br]

To a stirred solution of ethyl 2-(4-bromo-1H-pyrazol-3-yl)acetate (#123_2, 2.52 g, 10.5 mmol) in dry toluene (15.0 mL) was added 2,3-dihydro-4H-pyran (1.20 mL, 13.2 mmol) and TFA (50 μL, 0.673 mmol) and the reaction mixture was stirred at 110° C. for 3 h then allowed to cool to room temperature. The reaction mixture was diluted with saturated aqueous NaHCO₃ (150 mL) and EtOAc (30 mL). The layers were separated, and the aqueous layer was extracted with EtOAc (2×30 mL). The combined organic extracts were washed with brine (50 mL), dried over sodium sulfate, filtered and concentrated under vacuum. Purification by flash chromatography on silica gel (using a gradient of 0 to 100% MTBE in iso-hexane as eluent) gave the title compound (2.81 g, yield: 80%) as a yellow oil. LC-MS (Method B5′) m/z: [M+H]⁺: 317/319; rt: 2.01 min; purity>99%. ¹H NMR (500 MHz, DMSO-d₆) δ 8.12 (s, 1H), 5.33 (dd, J=10.1, 2.4 Hz, 1H), 4.10 (q, J=7.1 Hz, 2H), 3.95-3.87 (m, 1H), 3.66-3.56 (m, 3H), 2.13-1.99 (m, 1H), 1.96-1.83 (m, 2H), 1.71-1.58 (m, 1H), 1.57-1.46 (m, 2H), 1.19 (t, J=7.1 Hz, 3H).

### Step 4: Synthesis of 14-fluoro-12-methyl-4-tetrahydropyran-2-yl-4,5,9,13-tetrazatricyclo[8.4.0.0^{2,6}]tetradeca-1(10),2,5,11,13-pentaen-8-one #123_4

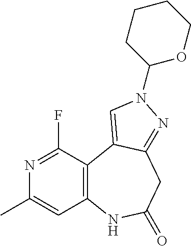 [SMILES: Cc1cc2c(c(F)n1)-c1cn(C3CCCCO3)nc1CC(=O)N2]

To a microwave vial containing ethyl 2-(4-bromo-2-tetrahydropyran-2-yl-pyrazol-3-yl)acetate (#123_3, 157 mg, 0.470 mmol) were added 1,4-dioxane (10.0 mL), water (0.75 mL), 2-fluoro-6-methyl-3-(4,4,5,5-tetramethyl-1,3,2-dioxaborolan-2-yl)pyridin-4-amine (Intermediate N16_1, 272 mg, 0.971 mmol) and cesium fluoride (219 mg, 1.44 mmol) and the resulting mixture was degassed with nitrogen for 5 min. Bis(di-tert-butyl(4-dimethylaminophenyl)phosphine)dichloropalladium(II) (37 mg, 0.052 mmol) was added and the reaction mixture was degassed again with nitrogen for 5 min then irradiated under microwaves at 120° C. for 2 h. After cooling to room temperature, the reaction mixture was diluted with EtOAc (20 mL), filtered through Celite® and the filter cake was washed with EtOAc (5×30 mL). The filtrate was concentrated under vacuum and the residue was dissolved in toluene (20 mL), cooled to 0° C. and a 1 M solution of lithium bis(trimethylsilyl)amide in THF (1.50 mL, 1.50 mmol) was added dropwise. The reaction mixture was stirred at room temperature overnight before being diluted with saturated aqueous NH₄Cl (200 mL) and EtOAc (50 mL). The layers were separated and the aqueous layer was extracted with EtOAc (2×50 mL). The combined organic extracts were washed with saturated aqueous NaHCO₃ solution (50 mL), brine (50 mL), dried over sodium sulfate, filtered and concentrated under vacuum. Purification by flash chromatography on silica gel (using a gradient of 0 to 100% EtOAc in iso-hexane as eluent) gave the title compound (57 mg, yield: 37%) as a white solid. LC-MS (Method B5′) m/z: [M+H]⁺: 317; rt: 1.49 min; purity: 98%. ¹H NMR (400 MHz, DMSO-d₆) δ 10.56 (s, 1H), 8.20 (d, J=3.9 Hz, 1H), 6.92 (s, 1H), 5.46 (dd, J=10.1, 2.3 Hz, 1H), 3.94 (d, J=11.8 Hz, 1H), 3.68-3.58 (m, 3H), 2.38 (s, 3H), 2.25-2.05 (m, 1H), 1.93 (d, J=11.3 Hz, 2H), 1.74-1.59 (m, 1H), 1.55 (dd, J=8.0, 3.9 Hz, 2H).

### Step 5: Synthesis of 2-(14-fluoro-12-methyl-8-oxo-4-tetrahydropyran-2-yl-4,5,9,13-tetrazatricyclo[8.4.0.0^{2,6}]tetradeca-1(10),2,5,11,13-pentaen-9-yl)-N-[4-(trifluoromethyl)phenyl]acetamide #123_5

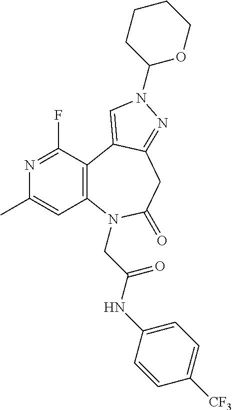 [SMILES: Cc1cc2c(c(F)n1)-c1cn(C3CCCCO3)nc1CC(=O)N2CC(=O)Nc1ccc(C(F)(F)F)cc1]

The title product was prepared according to general procedure 1, starting from 14-fluoro-12-methyl-4-tetrahydropyran-2-yl-4,5,9,13-tetrazatricyclo[8.4.0.0^{2,6}]tetradeca-1(10),2,5,11,13-pentaen-8-one (#123_4, 57 mg, 0.177 mmol) and 2-chloro-N-(4-trifluoromethyl-phenyl)acetamide (47 mg, 0.200 mmol). Purification by flash chromatography on silica gel (using a gradient of 0 to 100% EtOAc in iso-hexane as eluent) afforded the title compound (85 mg, yield: 88%) as a yellow solid. LC-MS (Method B5′) m/z: [M+Na]⁺: 540, [M+H]⁺: 518; rt: 2.23 min; purity: 98%. ¹H NMR (400 MHz, DMSO-d₆) δ 10.61 (s, 1H), 8.28 (d, J=3.5 Hz, 1H), 7.81 (d, J=8.6 Hz, 2H), 7.69 (d, J=8.6 Hz, 2H), 7.29 (s, 1H), 5.48 (dd, J=10.4, 2.1 Hz, 1H), 4.55 (s, 2H), 3.96 (d, J=11.6 Hz, 1H), 3.70-3.57 (m, 3H), 2.44 (s, 3H), 2.24-2.08 (m, 1H), 1.96 (app. d, J=11.2 Hz, 2H), 1.74-1.63 (m, 1H), 1.59-1.51 (m, 2H). ¹⁹F NMR (376 MHz, DMSO-d₆) δ−60.29, −70.35.

### Step 6: Synthesis of 2-(14-fluoro-12-methyl-8-oxo-4,5,9,13-tetrazatricyclo[8.4.0.0^{2,6}]tetradeca-1(10),2,5,11,13-pentaen-9-yl)-N-[4-(trifluoromethyl)phenyl]acetamide #123

To a solution of 2-(14-fluoro-12-methyl-8-oxo-4-tetrahydropyran-2-yl-4,5,9,13-tetrazatricyclo[8.4.0.0^{2,6}]tetradeca-1(10),2,5,11,13-pentaen-9-yl)-N-[4-(trifluoromethyl)phenyl]acetamide (#123_5, 85 mg, 0.148 mmol) in DCM (2.0 mL) was added trifluoroacetic acid (1.0 mL, 13.5 mmol). The reaction mixture was stirred at room temperature for 1 h, then neutralized carefully with saturated aqueous NaHCO₃ (50 mL), diluted with DCM (30 mL) and the layers were separated. The aqueous layer was extracted with DCM (2×30 mL). The combined organic extracts were washed with brine (20 mL), dried over sodium sulfate, filtered and concentrated under vacuum. Purification by flash chromatography on silica gel (using a gradient of 0 to 100% EtOAc in iso-hexane as eluent) gave the title compound (50 mg, yield: 77%) as a white solid. ¹⁹F NMR (376 MHz, DMSO-d₆) δ−60.29, −70.43. LC-MS (Method A7): m/z [M+H]⁺: 434.1; rt: 1.95 min; purity: 99%. ¹H NMR (400 MHz, DMSO-d₆) δ 13.26 (s, 1H), 10.60 (s, 1H), 8.15 (s, 1H), 7.81 (d, J=8.5 Hz, 2H), 7.69 (d, J=8.6 Hz, 2H), 7.28 (s, 1H), 4.54 (s, 2H), 3.61 (s, 2H), 2.44 (s, 3H).

### Example #124: 2-(3-fluoro-5-methoxy-9-oxo-4,8,12-triazatricyclo[9.4.0.0^{2,7}]pentadeca-1(11),2,4,6,12,14-hexaen-8-yl)-N-[4-(trifluoromethyl)phenyl]acetamide

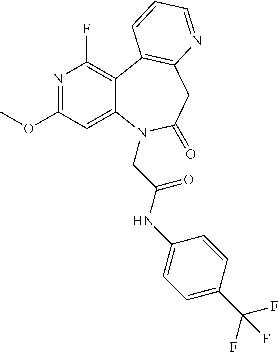 [SMILES: COc1cc2c(c(F)n1)-c1cccnc1CC(=O)N2CC(=O)Nc1ccc(C(F)(F)F)cc1]

The title product was prepared according to general procedure 1, starting from Intermediate N40 and 2-chloro-N-(4-trifluoromethyl-phenyl)acetamide. Purification by flash chromatography on silica gel (using a gradient of 0-5% MeOH/DCM as eluent) and reverse phase preparative HPLC (HSS PFP column C18, using a gradient of 0.1% Formic Acid in H₂O/MeCN as eluent) gave the title compound (33 mg, yield: 38%) as a white solid. LC-MS (Method B5): m/z [M+H]⁺: 461.2; rt: 2.20 min; purity: >99%. ¹H NMR (400 MHz, DMSO-d₆) δ 10.62 (s, 1H), 8.58 (dd, J=4.8, 1.7 Hz, 1H), 8.11 (ddd, J=8.0, 4.7, 1.7 Hz, 1H), 7.77 (d, J=8.5 Hz, 2H), 7.68 (d, J=8.5 Hz, 2H), 7.50 (dd, J=7.9, 4.8 Hz, 1H), 6.90 (s, 1H), 4.61 (s, 2H), 3.95 (d, J=12.6 Hz, 1H), 3.92 (s, 3H), 3.70 (d, J=12.6 Hz, 1H).

### Example #125: 2-(4-ethyl-5-methyl-3,9-dioxo-4,8,12-triazatricyclo[9.4.0.0^{2,7}]pentadeca-1(15),2(7),5,11,13-pentaen-8-yl)-N-[4-(trifluoromethyl)phenyl]acetamide

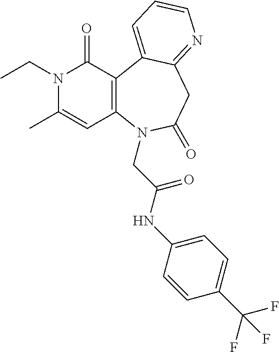 [SMILES: CCn1c(C)cc2c(c1=O)-c1cccnc1CC(=O)N2CC(=O)Nc1ccc(C(F)(F)F)cc1]

The title product was prepared according to general procedure 1, starting from Intermediate N41 and 2-chloro-N-(4-trifluoromethyl-phenyl)acetamide. Purification by column chromatography on silica gel (using a gradient of 0-10% MeOH/DCM as eluent) afforded the title compound (1 mg, yield: 32%) as a white solid. LC-MS (Method A7): m/z [M+H]⁺: 471.1; rt: 1.78 min; purity: 96%. ¹H NMR (400 MHz, CD30D) δ 8.52 (dd, J=5.0, 1.7 Hz, 1H), 8.42 (dd, J=7.9, 1.7 Hz, 1H), 7.79 (d, J=8.6 Hz, 2H), 7.64 (d, J=8.5 Hz, 2H), 7.49 (dd, J=8.1, 4.9 Hz, 1H), 6.54 (s, 1H), 4.59-4.46 (m, 2H), 4.25 (p, J=6.8 Hz, 2H), 3.91-3.79 (m, 2H), 2.57 (s, 3H), 1.39 (d, J=7.1 Hz, 3H). Proton for NH not observed.

### Example #126: 2-(3-chloro-5,10-dimethyl-9-oxo-4,8,10,12-tetrazatricyclo[9.4.0.0^{2,7}]pentadeca-1(11),2,4,6,12,14-hexaen-8-yl)-N-[4-(trifluoromethyl)phenyl]acetamide

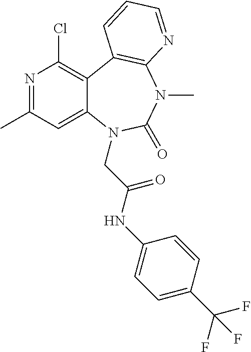 [SMILES: Cc1cc2c(c(Cl)n1)-c1cccnc1N(C)C(=O)N2CC(=O)Nc1ccc(C(F)(F)F)cc1]

The title product was prepared according to general procedure 1, starting from Intermediate N42 and 2-chloro-N-(4-trifluoromethyl-phenyl)acetamide and heating at 100° C. for 16 hours. Purification by preparative reverse phase HPLC on C18 silica gel (CSH column C18, using 0.1% Formic Acid in H₂O/MeCN as eluent) afforded the title compound (56 mg, yield: 23%) as a white solid. LC-MS(Method A7): m/z [M+H]⁺: 476.1/478.1; rt: 1.58 min, purity>99%. ¹H NMR (400 MHz, CD30D) δ 8.52 (dd, J=4.8, 1.8 Hz, 1H), 8.25 (dd, J=7.8, 1.8 Hz, 1H), 7.74 (d, J=8.5 Hz, 2H), 7.62 (d, J=8.6 Hz, 2H), 7.43-7.35 (m, 2H), 4.65 (d, J=16.3 Hz, 1H), 4.40 (d, J=16.3 Hz, 1H), 3.28 (s, 3H), 2.58 (s, 3H). Proton for NH not observed.

### Example #127: 2-(1-cyano-10-fluoro-3-methyl-6-oxo-7H-pyrido[4,3-d][3]benzazepin-5-yl)-N-[4-(trifluoromethyl)phenyl]acetamide

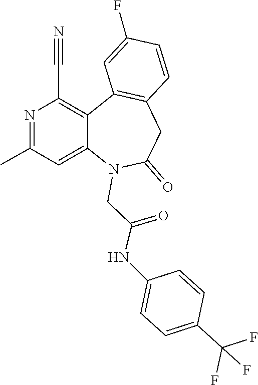 [SMILES: Cc1cc2c(c(C#N)n1)-c1cc(F)ccc1CC(=O)N2CC(=O)Nc1ccc(C(F)(F)F)cc1]

The title product was prepared according to general procedure 1 starting from Intermediate N108 and 2-chloro-N-(4-trifluoromethylphenyl)acetamide (CAS 2707-23-5). LC-MS (Method A7): m/z [M+H]⁺: 469.1; rt: 2.32 min; purity>99%. ¹H NMR (400 MHz, CD30D) δ 7.82-7.77 (m, 2H), 7.75-7.70 (m, 2H), 7.68-7.62 (m, 2H), 7.56-7.50 (m, 1H), 7.40-7.32 (m, 1H), 4.73-4.47 (m, 2H), 3.75-3.57 (m, 2H), 2.68 (s, 3H). Proton for NH not observed.

### Example #128: 2-(3-fluoro-5,10-dimethyl-9-oxo-4,8,12-triazatricyclo[9.4.0.0^{2,7}]pentadeca-1(11),2(7),3,5,12,14-hexaen-8-yl)-N-(3-fluoro-4-methyl-phenyl)acetamide

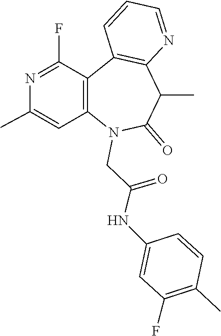

Step 1: Synthesis of tert-butyl 2-(3-fluoro-5,10-dimethyl-9-oxo-4,8,12-triazatricyclo[9.4.0.0^{2,7}]pentadeca-1(11),2(7),3,5,12,14-hexaen-8-yl)acetate #128_1

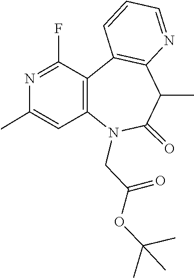

To a solution of Intermediate N23 (1.05 g, 3.47 mmol) in dry DMF (17 mL) were added tert-butyl bromoacetate (627 μL, 4.16 mmol), potassium carbonate (970 mg, 6.90 mmol) and potassium iodide (58 mg, 0.35 mmol) and the resulting mixture was stirred at room temperature for 20 h. Water was added and the reaction mixture was extracted with EtOAc. The combined organic layers were washed with brine, dried over Na₂SO₄, filtered off and concentrated to dryness. The crude orange oil was purified by flash chromatography on silica gel (using a gradient of heptane/EtOAc from 100/0 to 0/100) to afford the title compound (1.22 g, yield: 92%) as a yellow solid. LC-MS (Method-A1) m/z: [M+H]⁺: 372.4, rt: 1.33 min, purity: 97%. ¹H NMR (400 MHz, DMSO-d₆) δ 8.66 (dd, J=4.8, 1.6 Hz, 1H), 8.11 (ddd, J=7.9, 4.8, 1.6 Hz, 1H), 7.48 (dd, J=7.9, 4.8 Hz, 1H), 7.29 (s, 1H), 4.65-4.30 (m, 2H), 3.74 (d, J=6.6 Hz, 1H), 2.52 (s, 3H), 1.49 (d, J=6.6 Hz, 3H), 1.24 (s, 9H).

### Step 2: Synthesis of 2-(3-fluoro-5,10-dimethyl-9-oxo-4,8,12-triazatricyclo[9.4.0.0^{2,7}]pentadeca-1(11),2(7),3,5,12,14-hexaen-6-yl)acetic acid #128_2

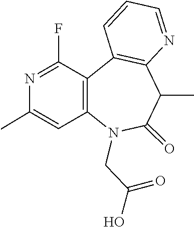

To a solution of tert-butyl 2-(1-fluoro-3,7-dimethyl-6-oxo-7H-dipyrido[2,3-b:3′,4′-d]azepin-5-yl)acetate (#128_1, 1.06 g, 2.80 mmol) in dry dichloromethane (14 mL) was added a 4 M solution of HCl in 1,4-dioxane (7 mL, 28 mmol) and the reaction mixture was stirred at room temperature for 20 h. The reaction mixture was concentrated to dryness and dried under vacuum to afford the title compound (1.13 g, quantitative yield) as a beige solid. LC-MS (Method-A1) m/z: [M+H]⁺: 316.2, rt: 0.93 min, purity: 97%. ¹H NMR (400 MHz, DMSO-de) δ 8.66 (dd, J=4.8, 1.7 Hz, 1H), 8.11 (ddd, J=7.9, 4.8, 1.7 Hz, 1H), 7.49 (dd, J=7.9, 4.8 Hz, 1H), 7.32 (s, 1H), 4.44 (s, 2H), 3.77 (q, J=6.6 Hz, 1H), 2.51 (s, 3H), 1.49 (d, J=6.6 Hz, 3H).

### Step 3: Synthesis of 2-(3-fluoro-5,10-dimethyl-9-oxo-4,8,12-triazatricyclo[9.4.0.02,]pentadeca-1(11),2(7),3,5,12,14-hexaen-8-yl)-N-(3-fluoro-4-methyl-phenyl)acetamide #128

To a solution of 2-(1-fluoro-3,7-dimethyl-6-oxo-7H-dipyrido[2,3-b:3′,4′-d]azepin-5-yl)acetic acid (#128_2, 15 mg, 0.047 mmol) and HATU (36 mg, 0.095 mmol) in dry DMF (0.25 mL) was added 3-fluoro-4-methyl-aniline (12 ma. 0.095 mmol), followed by diisopropylethylamine (47 μL. 0.285 mmol) and the reaction mixture was stirred at room temperature for 2 h. The reaction mixture was directly purified by reverse phase chromatography (with acidic elution) to afford the title compound (13 mg, yield: 65%) as a white solid. LC-MS (Method A4′) m/z [M+H]⁺: 423.2; rt: 2.51 min; purity>99%.

The following Examples #1294133 and #135-#136 have been prepared according to a similar procedure as the one depicted for example #128, starting from 2-(3-fluoro-5,1-dimethyl-9-oxo-4,8,12-triazatricyclo[9.4.0.0^{2,7}]pentadeca-1(11)2(7)3,5,12,14-hexaen-8-yl)acetic acid #128_2 and different anilines, as referenced hereunder. Example #134 has been prepared according to a similar procedure as the one depicted for example #128, starting from 2-(1-fluoro-3-methyl-6-oxo-71H-pyrido[4,3-d][3]benzazepin-5-yl)acetic acid (prepared following the same procedure as the one depicted for Intermediate #128_2 but from Intermediate N113 instead of Intermediate N23).

| | Structure | | |
|---|---|---|---|
| Ex. | Example | Example Name | Protocolumn and characterization |
| #129 | 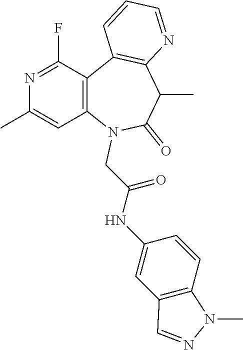 | 2-(3-fluoro-5,10- dimethyl-9-oxo-4,8,12- triazatricyclo[9.4.0.0^{2,7}]pen- tadeca- 1(11),2(7),3,5,12,14- hexaen-8-yl)-N-(1- methylindazol-5- yl)acetamide | Starting from 1-methylindazol-5-amine LC-MS (Method A4′) m/z [M + H]⁺: 445.2; rt: 2.05 min; purity = 97%. |
| #130 | 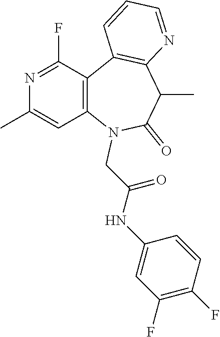 [SMILES: Cc1cc2c(c(F)n1)-c1cccnc1C(C)C(=O)N2CC(=O)Nc1ccc(F)c(F)c1] | N-(3,4-difluorophenyl)-2- (3-fluoro-5,10-dimethyl- 9-oxo-4,8,12- triazatricyclo[9.4.0.0^{2,7}]pen- tadeca- 1(11),2(7),3,5,12,14- hexaen-8-yl)acetamide | Starting from 3,4-difluoroaniline LC-MS (Method A4′) m/z [M + H]⁺: 427.1; rt: 2.41 min; purity >99% |
| #131 | 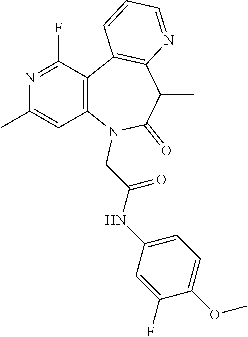 | 2-(3-fluoro-5,10- dimethyl-9-oxo-4,8,12- triazatricyclo[9.4.0.0^{2.7}]pen- tadeca- 1(11),2(7),3,5,12,14- hexaen-8-yl)-N-(3- fluoro-4-methoxy- phenyl)acetamide | Starting from 3-fluoro-4-methoxy-aniline LC-MS (Method A4′) m/z [M + H]⁺: 439.2; rt: 2.29 min; purity >99% |
| #132 | 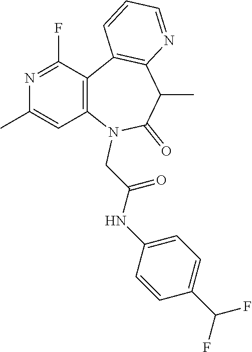 [SMILES: Cc1cc2c(c(F)n1)-c1cccnc1C(C)C(=O)N2CC(=O)Nc1ccc(C(F)F)cc1] | N-[4- (difluoromethyl)phenyl]- 2-(3-fluoro-5,10- dimethyl-9-oxo-4,8,12- triazatricyclo[9.4.0.0^{2,7}]pen- tadeca- 1(11),2(7),3,5,12,14- hexaen-8-yl)acetamide | Starting from 4-(difluoromethyl)aniline LC-MS (Method A4′) m/z [M + H]⁺: 441.2; rt: 2.40 min; purity = 96% |
| #133 | 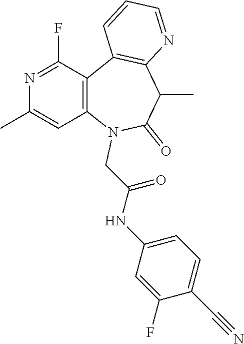 [SMILES: Cc1cc2c(c(F)n1)-c1cccnc1C(C)C(=O)N2CC(=O)Nc1ccc(C#N)c(F)c1] | N-(4-cyano-3-fluoro- phenyl)-2-(3-fluoro-5,10- dimethyl-9-oxo-4,8,12- triazatricyclo[9.4.0.0^{2,7}]pen- tadeca- 1(11),2(7),3,5,12,14- hexaen-8-yl)acetamide | Starting from 4-amino-2-fluoro-benzonitrile LC-MS (Method A4′) m/z [M + H]⁺: 434.1; rt: 2.37 min; purity = 98% |
| #134 | 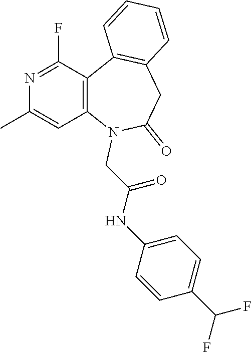 [SMILES: Cc1cc2c(c(F)n1)-c1ccccc1CC(=O)N2CC(=O)Nc1ccc(C(F)F)cc1] | N-[4- (difluoromethyl)phenyl]- 2-(1-fluoro-3-methyl-6- oxo-7H-pyrido[4,3- d][3]benzazepin-5- yl)acetamide | Starting from 4-(difluoromethyl)aniline LC-MS (Method A4′) m/z [M + H]⁺: 426.1; rt: 2.55 min; purity >99% |
| #135 | 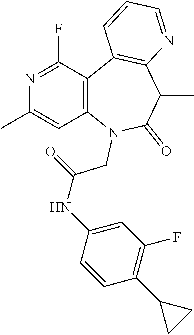 | N-(4-cyclopropyl-3- fluoro-phenyl)-2-(3- fluoro-5,10-dimethyl-9- oxo-4,8,12- triazatricyclo[9.4.0.0^{2,7}]pen- tadeca- 1(11),2(7),3,5,12,14- hexaen-8-yl)acetamide | Starting from 4-cyclopropyl-3-fluoro-aniline LC-MS (Method B4) m/z [M + H]⁺: 449.6; rt: 4.14 min; purity: 95%. LC-MS (Method A4) m/z [M + H]⁺: 449.6; rt: 4.62 min; purity: 95%. ¹H NMR (400 MHz, DMSO-d₆) δ 10.33 (s, 1H), 8.67 (dd, J = 4.8, 1.7 Hz, 1H), 8.12 (ddd, J = 7.9, 4.8, 1.7 Hz, 1H), 7.54-7.43 (m, 2H), 7.36 (s, 1H), 7.16 (dd, J = 8.5, 2.2 Hz, 1H), 6.93 (t, J = 8.7 Hz, 1H), 4.58-4.49 (m, 2H), 3.77 (q, J = 6.6 Hz, 1H), 2.52 (s, 3H), 1.96 (td, J = 8.5, 4.3 Hz, 1H), 1.49 (d, J = 6.6 Hz, 3H), 0.97-0.83 (m, 2H), 0.70-0.62 (m, 2H). |
| #136 | 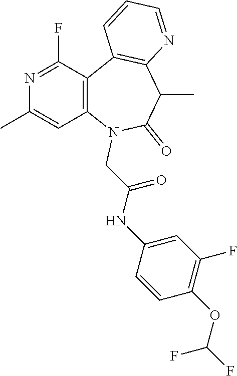 [SMILES: Cc1cc2c(c(F)n1)-c1cccnc1C(C)C(=O)N2CC(=O)Nc1ccc(OC(F)F)c(F)c1] | N-[4-(difluoromethoxy)- 3-fluoro-phenyl]-2-(3- fluoro-5,10-dimethyl-9- oxo-4,8,12- triazatricyclo[9.4.0.0^{2,7}]pen- tadeca- 1(11),2(7),3,5,12,14- hexaen-8-yl)acetamide | Starting from 4-(difluoromethoxy)-3-fluoro- aniline LC-MS (Method A4′) m/z [M + H]⁺: 475.1; rt: 2.50 min; purity >99% |

The following Examples #137-#151 have been prepared according to a similar procedure as the one depicted for example #128, but starting from 2-[(10R)-3-fluoro-5,10-dimethyl-9-oxo-4,8,12-triazatricyclo[9.4.0.0^{2,7}]pentadeca-1 (11),2(7),3,5,12,14-hexaen-8-yl]acetic acid (intermediate N62) and different anilines, as referenced here under.

| Ex. | Structure Example | Example Name | Protocolumn and characterization |
|---|---|---|---|
| #137 | 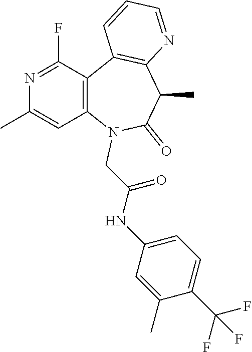 [SMILES: Cc1cc2c(c(F)n1)-c1cccnc1[C@@H](C)C(=O)N2CC(=O)Nc1ccc(C(F)(F)F)c(C)c1] | 2-[(10R)-3-fluoro- 5,10-dimethyl-9-oxo- 4,8,12- triazatricyclo[9.4.0.0^{2,7}] pentadeca- 1(11),2(7),3,5,12,14- hexaen-8-yl]-N-[3- methyl-4- (trifluoromethyl)phenyl] acetamide | Starting from 3-methyl-4- (trifluoromethyl)aniline LC-MS (Method A4′) m/z [M + H]⁺: 473.1; rt: 2.71 min; purity: 97% |
| #138 | 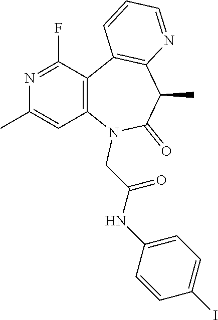 [SMILES: Cc1cc2c(c(F)n1)-c1cccnc1[C@@H](C)C(=O)N2CC(=O)Nc1ccc(I)cc1] | 2-[(10R)-3-fluoro- 5,10-dimethyl-9-oxo- 4,8,12- triazatricyclo[9.4.0^{2,7}] pentadeca- 1(11),2(7),3,5,12,14- hexaen-8-yl]-N-(4- iodophenyl)acetamide | Starting from 4-iodoaniline LC-MS (Method A4′) m/z [M + H]⁺: 517.0; rt: 2.59 min; purity: 94% |
| #139 | 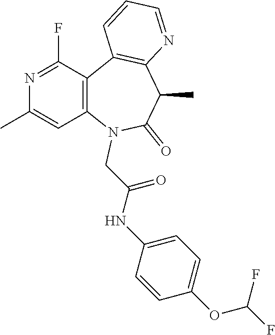 [SMILES: Cc1cc2c(c(F)n1)-c1cccnc1[C@@H](C)C(=O)N2CC(=O)Nc1ccc(OC(F)F)cc1] | N-[4- (difluoromethoxy)phe- nyl]-2-[(10R)-3- fluoro-5,10-dimethyl- 9-oxo-4,8,12- triazatricyclo[9.4.0.0^{2,7}] pentadeca- 1(11),2(7),3,5,12,14- hexaen-8- yl]acetamide | Starting from 4-(difluoromethoxy)aniline LC-MS (Method A4′) m/z [M + H]⁺: 457.1; rt: 2.40 min; purity: 94% |
| #140 | 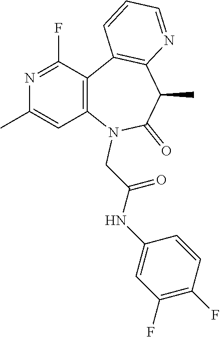 [SMILES: Cc1cc2c(c(F)n1)-c1cccnc1[C@@H](C)C(=O)N2CC(=O)Nc1ccc(F)c(F)c1] | N-(3,4- difluorophenyl)-2- [(10R)-3-fluoro-5,10- dimethyl-9-oxo- 4,8,12- triazatricyclo[9.4.0.0^{2,7}] pentadeca- 1(11),2(7),3,5,12,14- hexaen-8- yl]acetamide | Starting from 3,4-difluoroaniline LC-MS (Method A4′) m/z [M + H]⁺: 427.1; rt: 2.41 min; purity 98% |
| #141 | 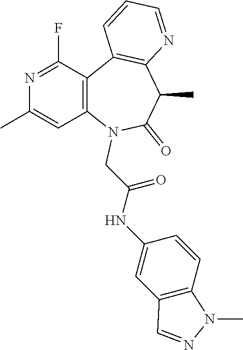 | 2-[(10R)-3-fluoro- 5,10-dimethyl-9- oxo-4,8,12- triazatricyclo[9.4.0.0^{2,7}] pentadeca- 1(11),2(7),3,5,12,14- hexaen-8-yl]-N-(1- methylindazol-5- yl)acetamide | Starting from 1-methylindazol-5-amine LC-MS (Method A4′) m/z [M + H]⁺: 445.2; rt: 2.09 min; purity = 93%. |
| #142 | 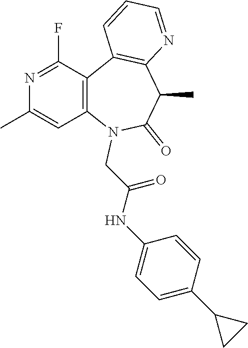 [SMILES: Cc1cc2c(c(F)n1)-c1cccnc1[C@@H](C)C(=O)N2CC(=O)Nc1ccc(C2CC2)cc1] | N-(4- cyclopropylphenyl)- 2-[(10R)-3-fluoro- 5,10-dimethyl-9- oxo-4,8,12- triazatricyclo[9.4.0.0^{2,7}] pentadeca- 1(11),2(7),3,5,12,14- hexaen-8- yl]acetamide | Starting from 4-cyclopropylaniline LC-MS (Method A4′) m/z [M + H]⁺: 431.2; rt: 2.52 min; purity: 92% |
| #143 | 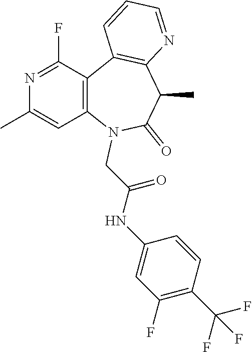 [SMILES: Cc1cc2c(c(F)n1)-c1cccnc1[C@@H](C)C(=O)N2CC(=O)Nc1ccc(C(F)(F)F)c(F)c1] | 2-[(10R)-3-fluoro- 5,10-dimethyl-9- oxo-4,8,12- triazatricyclo[9.4.0.0^{2,7}] pentadeca- 1(11),2(7),3,5,12,14- hexaen-8-yl]-N-[3- fluoro-4- (trifluoromethyl)phe- nyl]acetamide | Starting from 3-fluoro-4- (trifluoromethyl)aniline LC-MS (Method A4′) m/z [M + H]⁺: 477.1; rt: 2.67 min; purity: 93% |
| #144 | 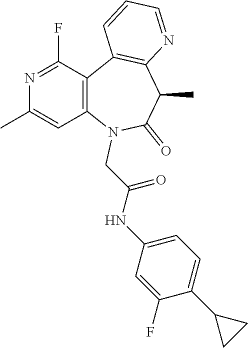 [SMILES: Cc1cc2c(c(F)n1)-c1cccnc1[C@@H](C)C(=O)N2CC(=O)Nc1ccc(C2CC2)c(F)c1] | N-(4-cyclopropyl-3- fluoro-phenyl)-2- [(10R)-3-fluoro- 5,10-dimethyl-9- oxo-4,8,12- triazatricyclo[9.4.0.0^{2,7}] pentadeca- 1(11),2(7),3,5,12,14- hexaen-8- yl]acetamide | Starting from 4-cyclopropyl-3-fluoro- aniline LC-MS (Method A4) m/z [M + H]⁺: 449.0; rt: 4.68 min; purity: 96%, LC-MS (Method B4) m/z [M + H]⁺: 449.0; rt: 4.48 min; purity: 99%. ¹H NMR (400 MHz, DMSO-d₆) δ 10.34 (s, 1H), 8.67 (dd, J = 4.8, 1.8 Hz, 1H), 8.13 (ddd, J = 7.9, 4.8, 1.8 Hz, 1H), 7.54-7.45 (m, 2H), 7.36 (s, 1H), 7.16 (dd, J = 8.5, 2.3 Hz, 1H), 6.94 (t, J = 8.6 Hz, 1H), 4.55 (d, J = 16.9 Hz, 1H), 4.54 (d, J = 16.9 Hz, 1H), δ 3.78 (q, J = 6.5 Hz, 1H), 2.51 (s, 3H), 2.03-1.91 (m, 1H), 1.49 (d, J = 6.5 Hz, 3H), 1.16-0.76 (m, 2H), 0.74-0.55 (m, 2H). |
| #145 | 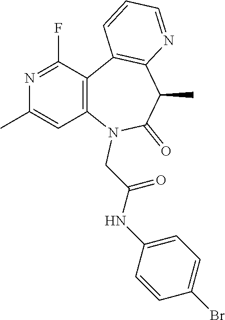 [SMILES: Cc1cc2c(c(F)n1)-c1cccnc1[C@@H](C)C(=O)N2CC(=O)Nc1ccc(Br)cc1] | N-(4-bromophenyl)- 2-[(10R)-3-fluoro- 5,10-dimethyl-9- oxo-4,8,12- triazatricyclo[9.4.0.0^{2,7}] pentadeca- 1(11),2(7),3,5,12,14- hexaen-8- yl]acetamide | Starting from 4-bromoaniline LC-MS (Method A4′) m/z [M + H]⁺: 469.0; rt: 2.52 min; purity: 94% |
| #146 | 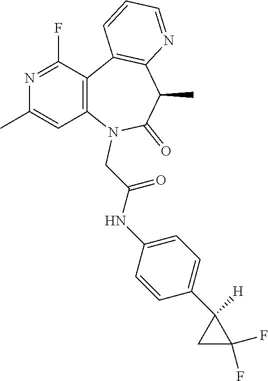 [SMILES: Cc1cc2c(c(F)n1)-c1cccnc1[C@@H](C)C(=O)N2CC(=O)Nc1ccc([C@@H]2CC2(F)F)cc1] | Enantiomer (1S) or (1R) of N-[4-(2,2- difluorocyclopropyl) phenyl]-2-[(10R)-3- fluoro-5,10- dimethyl-9-oxo- 4,8,12- triazatricyclo[9.4.0.0^{2,7}] pentadeca- 1(11),2(7),3,5,12,14- hexaen-8- yl]acetamide | From 4-(2,2-difluorocyclopropyl)aniline (Intermediate N74). The reaction mixture of the 2 diastereoisomers was separated by chiral chromatography (HPLC Chirlapak IB, Ethanol/n-heptane 20/80 (v/v)) Chiral purity: 93%; rt = 3.04 min (second eluting diastereoisomer). For information, first eluting diastereoisomer rt = 2.72 min. Both measured by HPLC, Chiralpak IB from Daicel, EtOH 30% heptane 70% DEA 0.1%). LC-MS (Method A2′) m/z ([M + H]⁺: 467.1; rt: 4.43 min; purity: 99%) |
| | or | | |
| | 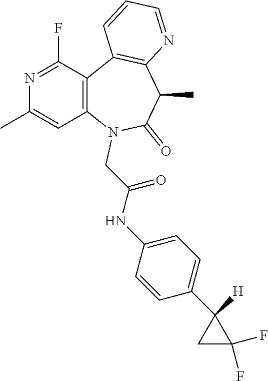 [SMILES: Cc1cc2c(c(F)n1)-c1cccnc1[C@@H](C)C(=O)N2CC(=O)Nc1ccc([C@H]2CC2(F)F)cc1] | | |
| #147 | 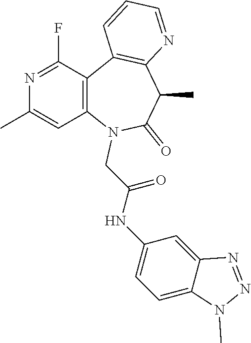 [SMILES: Cc1cc2c(c(F)n1)-c1cccnc1[C@@H](C)C(=O)N2CC(=O)Nc1ccc2c(c1)nnn2C] | 2-[(10R)-3-fluoro- 5,10-dimethyl-9- oxo-4,8,12- triazatricyclo[9.4.0.0^{2,7}] pentadeca- 1(11),2(7),3,5,12,14- hexaen-8-yl]-N-(1- methylbenzotriazol- 5-yl)acetamide | Starting from 1-methylbenzotriazol-5- amine. LC-MS (Method A4′) m/z [M + H]⁺: 446.1; rt: 1.92 min; purity: 93% |
| #148 | 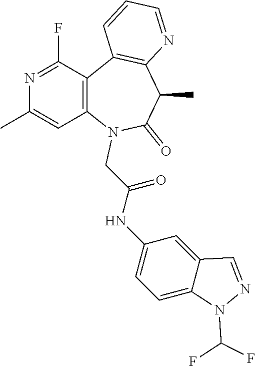 [SMILES: Cc1cc2c(c(F)n1)-c1cccnc1[C@@H](C)C(=O)N2CC(=O)Nc1ccc2c(cnn2C(F)F)c1] | N-[1- (difluoromethyl)inda- zol-5-yl]-2-[(10R)-3- fluoro-5,10- dimethyl-9-oxo- 4,8,12- triazatricyclo[9.4.0.0^{2,7}] pentadeca- 1(11),2(7),3,5,12,14- hexaen-8- yl]acetamide | Starting from 1-(difluoromethyl)indazol- 5-amine. LC-MS (Method A4′) m/z [M + H]⁺: 481.1; rt: 2.31 min; purity: 96% |
| #149 | 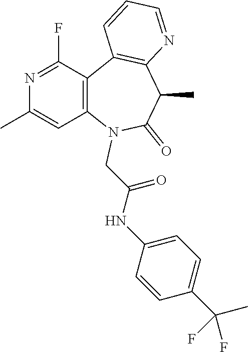 [SMILES: Cc1cc2c(c(F)n1)-c1cccnc1[C@@H](C)C(=O)N2CC(=O)Nc1ccc(C(C)(F)F)cc1] | N-[4-(1,1- difluoroethyl)phenyl]- 2-[(10R)-3-fluoro- 5,10-dimethyl-9- oxo-4,8,12- triazatricyclo[9.4.0.0^{2,7}] pentadeca- 1(11),2(7),3,5,12,14- hexaen-8- yl]acetamide | Starting from 4-(1,1-difluoroethyl)aniline LC-MS (Method A4') m/z [M + H]⁺: 455.1; rt: 2.50 min; purity: 97% |
| #150 | 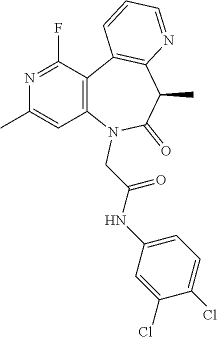 [SMILES: Cc1cc2c(c(F)n1)-c1cccnc1[C@@H](C)C(=O)N2CC(=O)Nc1ccc(Cl)c(Cl)c1] | N-(3,4- dichlorophenyl)-2- [(10R)-3-fluoro- 5,10-dimethyl-9- oxo-4,8,12- triazatricyclo[9.4.0.0^{2,7}] pentadeca- 1(11),2(7),3,5,12,14- hexaen-8- yl]acetamide | Starting from 3,4-dichloroaniline. LC-MS (Method A4) m/z [M + H]⁺: 459.1; rt: 4.77 min; purity: 99%, LC-MS (Method B4) m/z [M + H]⁺: 459.1; rt: 4.55 min; purity: 99%. ¹H NMR (400 MHz, DMSO-d₆) δ 10.55 (s, 1H), 8.67 (dd, J = 4.8, 1.7 Hz, 1H), 8.17-8.08 (m, 1H), 7.97 (d, J = 2.4 Hz, 1H), 7.57 (d, J = 8.8 Hz, 1H), 7.50 (dd, J = 7.9, 4.8 Hz, 1H), 7.44 (dd, J = 8.8, 2.4 Hz, 1H), 7.36 (s, 1H), 4.56 (s, 2H), 3.78 (q, J = 6.6 Hz, 1H), 2.51 (s, 3H), 1.49 (d, J = 6.6 Hz, 3H) |
| #151 | 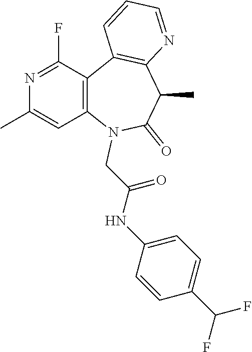 [SMILES: Cc1cc2c(c(F)n1)-c1cccnc1[C@@H](C)C(=O)N2CC(=O)Nc1ccc(C(F)F)cc1] | N-[4- (difluoromethyl)phe- nyl]-2-[(10R)-3- fluoro-5,10- dimethyl-9-oxo- 4,8,12- triazatricyclo[9.4.0.0^{2,7}] pentadeca- 1(11),2(7),3,5,12,14- hexaen-8- yl]acetamide | Starting from 4-(difluoromethyl)aniline. LC-MS (Method A4) m/z [M + H]⁺: 441.1; rt: 4.25 min; purity: 99%, LC-MS (Method B4) m/z [M + H]⁺: 441.1; rt: 4.20 min; purity: 99%. ¹H NMR (400 MHz, DMSO-d6) δ 10.46 (s, 1H), 8.67 (dd, J = 4.8, 1.8 Hz, 1H), 8.13 (ddd, J = 7.9, 4.8, 1.8 Hz, 1H), 7.69 (d, J = 8.7 Hz, 2H), 7.55-7.46 (m, 3H), 7.38 (s, 1H), 6.96 (t, J = 56.2 Hz, 1H), 4.59 (d, J = 16.8 Hz, 1H), 4.58 (d, J = 16.8 Hz, 1H), 3.78 (q, J = 6.6 Hz, 1H), 2.52 (s, 3H), 1.49 (d, J = 6.6 Hz, 3H). |

### Example #152: 2-[3-fluoro-10-(hydroxymethyl)-5-methyl-9-oxo-4,8,12-triazatricyclo[9.4.0.0^{2,7}]pentadeca-1(11),2(7),3,5,12,14-hexaen-8-yl]-N-[4-(trifluoromethyl)phenyl]acetamide

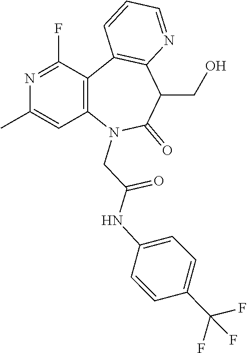 [SMILES: Cc1cc2c(c(F)n1)-c1cccnc1C(CO)C(=O)N2CC(=O)Nc1ccc(C(F)(F)F)cc1]

### Step 1: Synthesis of 2-[3-fluoro-5-methyl-9-oxo-10-(2-trimethylsilylethoxymethyl)-4,8,12-triazatricyclo[9.4.0.0²⁷]pentadeca-1(11),2,4,6,12,14-hexaen-8-yl]-N-[4-(trifluoromethyl)phenyl]acetamide #152_1

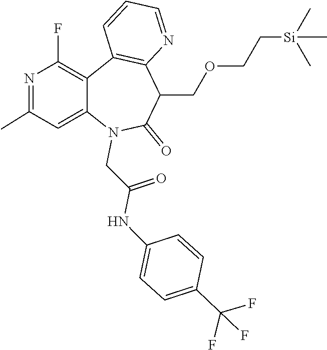

The title product was prepared according to general procedure 1, starting from 3-fluoro-5-methyl-10-(2-trimethylsilylethoxymethyl)-4,8,12-triazatricyclo[9.4.0.0^{2,7}]pentadeca-1 (15),2,4,6,11,13-hexaen-9-one Intermediate N75, 80 mg, 0.214 mmol) and 2-chloro-N-[4-(trifluoromethyl)phenyl]acetamide (CAS 2707-23-5, 61 mg, 0.257 mmol). Upon completion of reaction, water (20 mL) was added to the reaction mixture and the resulting precipitate was collected by filtration, washed with water (10 mL) and dried under vacuum to afford the title compound (90 mg, yield: 69%) as a white solid. LC-MS (Method A7) m/z: [M+H]⁺: 575; rt: 2.84 min; purity: 95%.

### Step 2: Synthesis of 2-[3-fluoro-10-(hydroxymethyl)-5-methyl-9-oxo-4,8,12-triazatricyclo[9.4.0.0^{2,7}]pentadeca-1(11),2(7),3,5,12,14-hexaen-8-yl]-N-[4-(trifluoromethyl)phenyl]acetamide #152

To a solution of 2-[3-fluoro-5-methyl-9-oxo-10-(2-trimethylsilylethoxymethyl)-4,8,12-triazatricyclo[9.4.0.0^{2,7}]pentadeca-1(11),2,4,6,12,14-hexaen-8-yl]-N-[4-(trifluoromethyl)phenyl]acetamide (#152_1, 80 mg, 0.139 mmol) in dichloromethane (2.0 mL) was added TFA (0.52 mL, 6.96 mmol) and the reaction mixture was stirred at room temperature for 2 hours. The reaction mixture was evaporated to dryness and the residue was redissolved in DCM (10 mL), which was washed with saturated aqueous sodium bicarbonate solution (10 mL). The organic layer was separated and dried by passing through a phase separation cartridge. The filtrate was concentrated to dryness and the residue was triturated with acetonitrile (10 mL) to give the title compound (63 mg, yield: 94%) as a white solid. LC-MS (Method A7) m/z: [M+H]⁺: 475.2; rt: 1.92 min; purity: 98%. ¹H NMR (400 MHz, DMSO-d₆) δ 10.60 (s, 1H), 8.65 (dd, J=4.8, 1.7 Hz, 1H), 8.15 (ddd, J=7.9, 4.6, 1.7 Hz, 1H), 7.75 (d, J=8.5 Hz, 2H), 7.67 (d, J=8.7 Hz, 2H), 7.50 (dd, J=8.0, 4.8 Hz, 1H), 7.44 (s, 1H), 4.69-4.58 (m, 2H), 4.51 (s, 1H), 4.28 (qd, J=11.2, 7.4 Hz, 2H), 3.64 (dd, J=7.4, 5.5 Hz, 1H), 2.51 (s, 3H).

### Example #153: 2-(3-cyano-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

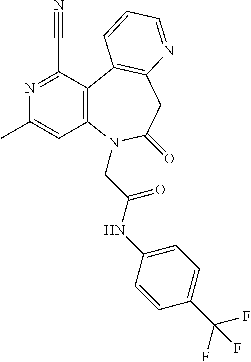 [SMILES: Cc1cc2c(c(C#N)n1)-c1cccnc1CC(=O)N2CC(=O)Nc1ccc(C(F)(F)F)cc1]

The title product was prepared according to general procedure 1 starting from Intermediate N128 and 2-chloro-N-(4-trifluoromethylphenyl)acetamide (CAS 2707-23-5) LC-MS (Method B2) m/z [M+H]⁺: 452.4; rt: 4.20 min; purity>99%. LC-MS (Method A2) m/z [M+H]⁺: 452.4; rt: 4.26 min; purity>99%. ¹H NMR (400 MHz, DMSO-d₆) δ 10.62 (s, 1H), 8.70 (dd, J=4.9, 1.6 Hz, 1H), 8.39 (dd, J=7.9, 1.6 Hz, 1H), 7.77 (d, J=8.8 Hz, 2H), 7.72 (s, 1H), 7.68 (d, J=8.8 Hz, 2H), 7.60 (dd, J=7.9, 4.9 Hz, 1H), 4.62 (s, 2H), 4.01 (d, J=12.7 Hz, 1H), 3.73 (d, J=12.7 Hz, 1H), 2.61 (s, 3H).

### Example #154: 2-(3-cyano-5,10-dimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

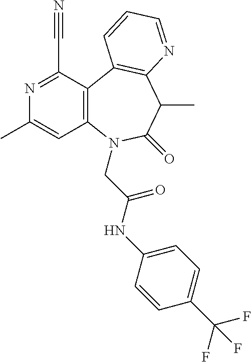 [SMILES: Cc1cc2c(c(C#N)n1)-c1cccnc1C(C)C(=O)N2CC(=O)Nc1ccc(C(F)(F)F)cc1]

The title product was prepared according to according to general procedure 1 starting from Intermediate N63 and 2-chloro-N-(4-trifluoromethylphenyl)acetamide (CAS 2707-23-5). LC-MS (Method A7) m/z [M+H]⁺: 466; rt: 2.21 min; purity>99%. ¹H NMR (400 MHz, DMSO-d₆) δ 10.62 (s, 1H), 8.74 (dd, J=4.8, 1.7 Hz, 1H), 8.35 (dd, J=7.9, 1.7 Hz, 1H), 7.80-7.64 (m, 5H), 7.58 (dd, J=7.9, 4.8 Hz, 1H), 4.70-4.49 (m, 2H), 3.89 (q, J=6.6 Hz, 1H), 2.61 (s, 3H), 1.49 (d, J=6.6 Hz, 3H).

### Example #155: Enantiomer (10R) or (10S) of N-(4-chloro-3-fluoro-phenyl)-2-[3-cyano-5,10-dimethyl-9-oxo-4,8,12-triazatricyclo[9.4.0.0^{2,7}]pentadeca-1(11),2(7),3,5,12,14-hexaen-8-yl]acetamide

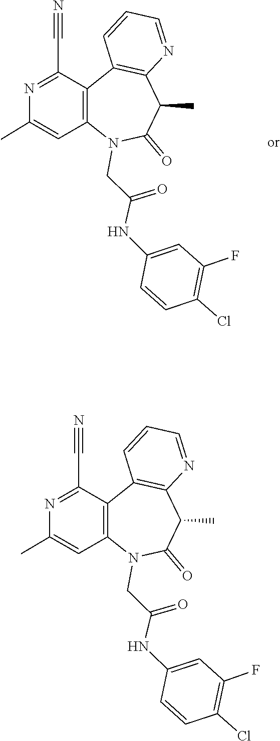 [SMILES: Cc1cc2c(c(C#N)n1)-c1cccnc1[C@@H](C)C(=O)N2CC(=O)Nc1ccc(Cl)c(F)c1 ; Cc1cc2c(c(C#N)n1)-c1cccnc1[C@H](C)C(=O)N2CC(=O)Nc1ccc(Cl)c(F)c1]

The title product was prepared according to general procedure 1 starting from intermediate N63 and 2-chloro-N-(4-chloro-3-fluoro-phenyl)acetamide (CAS 895641-02-8), followed by chiral separation (SFC-Chiralpak IG, CO₂+EtOH 20%). LC-MS (Method A4) m/z [M+H]⁺: 450.1; rt: 4.48 min; purity: 99%, LC-MS (Method B4) m/z [M+H]⁺: 450.1; rt: 4.32 min; purity: 99%. ¹H NMR (400 MHz, DMSO-d₆) δ 10.59 (s, 1H), 8.74 (dd, J=4.9, 1.8 Hz, 1H), 8.35 (dd, J=7.8, 1.8 Hz, 1H), 7.74 (d, J=2.4 Hz, 1H), 7.71 (s, 1H), 7.58 (dd, J=7.9, 4.8 Hz, 1H), 7.52 (t, J=8.8 Hz, 1H), 7.31 (dd, J=8.8, 2.4 Hz, 1H), 4.58 (s, 2H), 3.89 (q, J=6.6 Hz, 1H), 2.61 (s, 3H), 1.49 (d, J=6.6 Hz, 3H). Chiral purity: 98%; rt=2.21 min (second eluting enantiomer). For information, first eluting enantiomer rt=1.79 min. Both measured by HPLC, Chiralpak IG-u from Daicel, EtOH 50%-heptane 50%—DEA 0.1%).

### Example #156: 2-(3,7-dimethyl-1,6-dioxo-2,7-dihydropyrido[4,3-d][3]benzazepin-5-yl)-N-[4-(trifluoromethyl)phenyl]acetamide

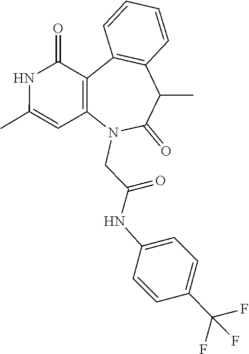 [SMILES: Cc1cc2c(c(=O)[nH]1)-c1ccccc1C(C)C(=O)N2CC(=O)Nc1ccc(C(F)(F)F)cc1]

### Step 1: Synthesis of 2-(1-methoxy-3,7-dimethyl-6-oxo-7H-pyrido[4,3-d][3]benzazepin-5-yl)-N-[4-(trifluoromethyl)phenyl]acetamide #156_1

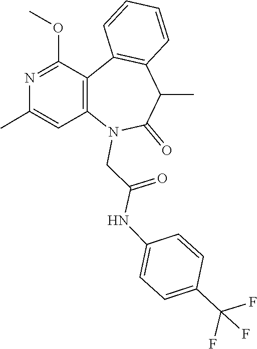 [SMILES: COc1nc(C)cc2c1-c1ccccc1C(C)C(=O)N2CC(=O)Nc1ccc(C(F)(F)F)cc1]

The title product was prepared according to general procedure 1, starting from 1-methoxy-3,7-dimethyl-5,7-dihydropyrido[4,3-d][3]benzazepin-6-one (Intermediate N64) and 2-chloro-N-(4-trifluoromethyl-phenyl)acetamide. Purification by flash chromatography on silica gel (using a gradient of 0 to 100% EtOAc in iso-hexane as eluent) afforded the title compound (170 mg, yield: 60%) as a pale-yellow solid. LC-MS (Method B5′) m/z: [M+H]⁺: 470; rt: 2.63 min; purity: 91%.

### Step 2: Synthesis of 2-(3,7-dimethyl-1,6-dioxo-2,7-dihydropyrido[4,3-d][3]benzazepin-5-yl)-N-[4-(trifluoromethyl)phenyl]acetamide #156_2

2-(1-Methoxy-3,7-dimethyl-6-oxo-7H-pyrido[4,3-d][3]benzazepin-5-yl)-N-[4-(trifluoromethyl)phenyl]acetamide (#156_1, 170 mg, 0.330 mmol) and potassium iodide (274 mg, 1.65 mmol) were suspended in dry acetonitrile (15 mL) then trimethylsilyl chloride (0.21 mL, 1.65 mmol) was added. The reaction mixture was stirred at 80° C. for 2 hours. The reaction mixture was concentrated under vacuum and the residue was partitioned between a solution of 10% MeOH in DCM (30 mL) and water (30 mL). The layers were separated and upon standing a precipitate formed in the aqueous phase which was collected by filtration and dried under vacuum to afford the title compound (92 mg, yield: 61%) as pale-yellow solid. LC-MS (Method B5′) m/z: [M+H]⁺: 456.2; rt: 1.95 min; purity>99%. ¹H NMR (400 MHz, DMSO-d₆) δ 11.82 (s, 1H), 10.52 (s, 1H), 7.86 (dd, J=7.8, 1.4 Hz, 1H), 7.78 (d, J=8.5 Hz, 2H), 7.68 (d, J=8.5 Hz, 2H), 7.40 (td, J=7.6, 1.5 Hz, 1H), 7.34-7.25 (m, 2H), 6.17 (s, 1H), 4.45-4.27 (m, 2H), 3.41 (q, J=6.7 Hz, 1H), 2.22 (s, 3H), 1.46 (d, J=6.7 Hz, 3H).

### Example #157: 2-(15-methoxy-8,13-dimethyl-9-oxo-4,6,10,14-tetrazatricyclo[9.4.0.0^{2,7}]pentadeca-1(11),2(7),3,5,12,14-hexaen-10-yl)-N-[4-(trifluoromethyl)phenyl]acetamide

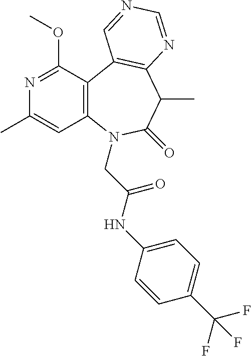 [SMILES: COc1nc(C)cc2c1-c1cncnc1C(C)C(=O)N2CC(=O)Nc1ccc(C(F)(F)F)cc1]

The title product was prepared according to general procedure 1, starting from Intermediate N43 and 2-chloro-N-(4-trifluoromethyl-phenyl)acetamide. Purification by flash chromatography on silica gel (using a gradient of 0-100% EtOAc/iso-hexane as eluent) afforded the title compound (83 mg, yield: 86%) as a white solid. LC-MS (Method A7): m/z [M+H]⁺: 472.2; rt: 2.28 min; purity: 95%. ¹H NMR (400 MHz, DMSO-d₆) δ 10.56 (s, 1H), 9.17 (s, 1H), 9.15 (s, 1H), 7.75 (d, J=8.5 Hz, 2H), 7.66 (d, J=8.5 Hz, 2H), 7.07 (s, 1H), 4.61-4.48 (m, 2H), 3.93 (s, 3H), 3.76 (q, J=6.6 Hz, 1H), 2.48 (s, 3H), 1.47 (d, J=6.6 Hz, 3H).

### Example #158: N-(4-bromo-3-fluoro-phenyl)-2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetamide

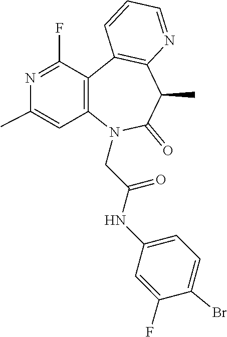 [SMILES: Cc1cc2c(c(F)n1)-c1cccnc1[C@@H](C)C(=O)N2CC(=O)Nc1ccc(Br)c(F)c1]

The title product was prepared according to general procedure 1, starting from Intermediate N61 and N-(4-bromo-3-fluoro-phenyl)-2-chloro-acetamide (CAS 1521715-10-5). LC-MS (Method A4) m/z [M+H]⁺: 487/489; rt: 4.06 min; purity: 99%, LC-MS (Method B4) m/z [M+H]⁺: 487/489; rt: 4.58 min; purity: 99%. ¹H NMR (400 MHz, DMSO-d₆) δ 10.58 (s, 1H), 8.67 (d, J=6.0 Hz, 1H), 8.16-8.09 (m, 1H), 7.71 (d, J=11.5 Hz, 1H), 7.63 (t, J=8.3 Hz, 1H), 7.54-7.46 (m, 1H), 7.37 (s, 1H), 7.26 (d, J=8.9 Hz, 1H), 4.56 (s, 2H), 3.78 (q, J=6.7 Hz, 1H), 2.50 (s, 3H), 1.49 (d, J=6.7 Hz, 3H).

### Example #159: 2-(3-chloro-10-methoxy-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

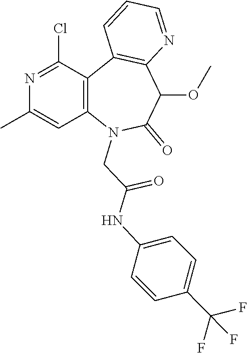 [SMILES: COC1C(=O)N(CC(=O)Nc2ccc(C(F)(F)F)cc2)c2cc(C)nc(Cl)c2-c2cccnc21]

### Step 1: Synthesis of 2-(3-fluoro-10-methoxy-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide #159_1

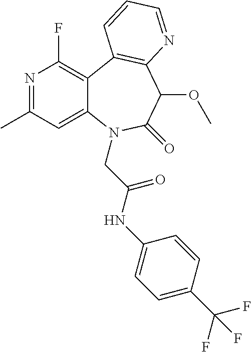 [SMILES: COC1C(=O)N(CC(=O)Nc2ccc(C(F)(F)F)cc2)c2cc(C)nc(F)c2-c2cccnc21]

The title product was prepared according to general procedure 1, starting from 3-fluoro-10-methoxy-5-methyl-4,8,12-triazatricyclo[9.4.0.0^{2,7}]pentadeca-1(15),2,4,6,11,13-hexaen-9-one (Intermediate N76, 190 mg, 0.695 mmol) and 2-chloro-N-[4-(trifluoromethyl)phenyl]acetamide (CAS 2707-23-5, 198 mg, 0.834 mmol). Upon completion of the reaction, water (20 mL) was added, and the resulting precipitate was collected by filtration. The precipitate was washed with water (10 mL) and dried under vacuum to afford the title compound (251 mg, yield: 71%) as a white solid. LC-MS (Method A7) m/z: [M+H]⁺: 475; rt: 1.97 and 2.19 min; purity: 98%.

### Step 2: Synthesis of 2-(3-chloro-10-methoxy-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide #159

A mixture of 2-(3-fluoro-10-methoxy-5-methyl-9-oxo-4,8,12-triazatricyclo[9.4.0.0^{2,7}]pentadeca-1(11),2,4,6,12,14-hexaen-8-yl)-N-[4-(trifluoromethyl)phenyl]acetamide (#159_1, 250 mg, 0.511 mmol) and a 4 M solution of HCl in 1,4-dioxane (5.0 mL, 20.0 mmol) in a sealed vial was heated at 100° C. for 1 hour. The reaction mixture was cooled to room temperature and carefully poured onto saturated sodium bicarbonate solution (~50 mL). The reaction mixture was extracted with EtOAc (2×30 mL), the organic extracts were combined and dried order sodium sulfate. Filtration and evaporation gave a solid which was purified by flash chromatography on silica gel (using EtOAc as eluent) to afford the title compound (83 mg, yield: 32%) as a white solid. LC-MS (Method A7) m/z: [M+H]⁺: 491/493; rt: 2.00 min; purity: 98%. ¹H NMR (400 MHz, DMSO-d₆) δ 10.60 (s, 1H), 8.67 (dd, J=4.8, 1.6 Hz, 1H), 8.23 (dd, J=7.9, 1.7 Hz, 1H), 7.81-7.61 (m, 4H), 7.51 (dd, J=8.0, 4.7 Hz, 1H), 7.44 (s, 1H), 5.04 (s, 1H), 4.65-4.52 (m, 2H), 3.39 (s, 3H), 2.53 (s, 3H).

### Example #160: 2-(3-chloro-12-fluoro-5-methyl-9-oxo-4,8,14-triazatricyclo[9.4.0.0^{2,7}]pentadeca-1(11),2(7),3,5,12,14-hexaen-8-yl)-N-[4-(trifluoromethyl)phenyl]acetamide

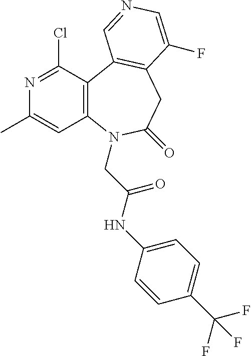 [SMILES: Cc1cc2c(c(Cl)n1)-c1cncc(F)c1CC(=O)N2CC(=O)Nc1ccc(C(F)(F)F)cc1]

A solution of 2-(3,12-difluoro-5-methyl-9-oxo-4-triazatricyclo[9.4.0.0^{2,7}]pentadeca-1(11),2(7),3,5,12,14-hexaen-8-yl)-N-[4-(trifluoromethyl)phenyl]acetamide (Example #212, 57 mg, 0.120 mmol) in a 4 M solution of HCl in 1,4-dioxane (1.0 mL, 4.00 mmol) was heated at 100° C. overnight. HCl in 1,4-dioxane (4 N, 1.0 mL, 4.00 mmol) was added again and the reaction mixture was heated for further 4 h at 100° C. The reaction mixture was cooled to room temperature, neutralized by addition of saturated aqueous NaHCO₃ (5 mL) and extracted with EtOAc (3×10 mL). The combined organic extracts were washed with brine (20 mL), dried over Na₂SO₄, filtered and concentrated under vacuum. The residue was purified by flash chromatography on silica gel (using a gradient of 0 to 10% MeOH in DCM as eluent) followed preparative HPLC (using 0.3% NH₃ in water/MeCN (30 to 100%) as eluent) to give the title compound (9 mg, yield: 14%) as a white solid. LC-MS (Method A7) m/z: [M+H]⁺: 479/481; rt: 2.18 min; purity: 90%. ¹H NMR (400 MHz, DMSO-d₆) δ 10.59 (s, 1H), 8.88 (s, 1H), 8.69 (s, 1H), 7.75 (d, J=8.5 Hz, 2H), 7.67 (d, J=8.5 Hz, 2H), 7.47 (s, 1H), 4.61 (d, J=16.9 Hz, 1H), 4.53 (d, J=16.9 Hz, 1H), 3.86 (d, J=13.1 Hz, 1H), 3.61 (d, J=13.1 Hz, 1H), 2.54 (s, 3H).

### Example #161: enantiomer (10R) or (10S) of 2-(3-hydroxy-5,10-dimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

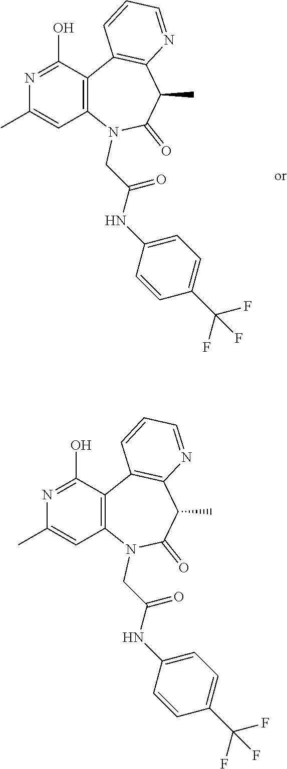 [SMILES: Cc1cc2c(c(O)n1)-c1cccnc1[C@@H](C)C(=O)N2CC(=O)Nc1ccc(C(F)(F)F)cc1 ; Cc1cc2c(c(O)n1)-c1cccnc1[C@H](C)C(=O)N2CC(=O)Nc1ccc(C(F)(F)F)cc1]

To a suspension of Example #192 (100 mg, 0.218 mmol) in 2-methoxyethanol (1.0 mL), was added a 4 M solution of hydrochloric acid in dioxane (0.1 mL, 0.393 mmol) and the resulting heterogeneous white mixture turned homogenous and was heated at 75° C. for 16 h. After cooling to room temperature, the reaction mixture was partitioned between EtOAc (10 mL) and a saturated solution of NaHCO₃ (25 mL) and was decanted. The organic layer was washed with water, dried with brine and over MgSO₄, filtered off and concentrated under vacuum. Purification by reverse phase chromatography (with basic elution) afforded the title compound (32 mg, yield: 32%) as a white solid. LC-MS (Method B4) m/z [M+H]⁺: 457.6; rt: 3.36 min; purity: 94%. LC-MS (Method A4) m/z [M+H]⁺: 457.6; rt: 3.59 min; purity: 91%. ¹H NMR (400 MHz, DMSO-d₆) δ 11.93 (s, 1H), 10.55 (s, 1H), 8.55 (dd, J=4.6, 1.9 Hz, 1H), 8.27 (dd, J=8.0, 1.9 Hz, 1H), 7.77 (d, J=8.8 Hz, 2H), 7.68 (d, J=8.8 Hz, 2H), 7.38 (dd, J=8.0, 4.6 Hz, 1H), 6.24 (s, 1H), 4.44 (s, 2H), 3.60 (q, J=6.7 Hz, 1H), 2.25 (s, 3H), 1.49 (d, J=6.7 Hz, 3H).

### Example #162: enantiomer (10R) or (10S) of 2-[3-(2-methoxyethoxy)-5,10-dimethyl-9-oxo-4,8,12-triazatricyclo[9.4.0.0^{2,7}]pentadeca-1(11),2(7),3,5,12,14-hexaen-8-yl]-N-[4-(trifluoromethyl)phenyl]acetamide

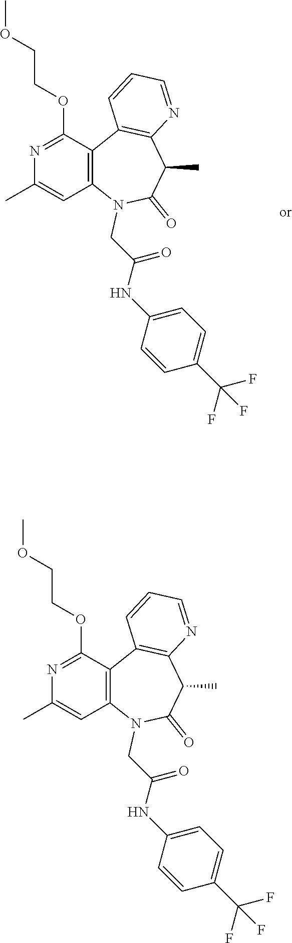 [SMILES: COCCOc1nc(C)cc2c1-c1cccnc1[C@@H](C)C(=O)N2CC(=O)Nc1ccc(C(F)(F)F)cc1 ; COCCOc1nc(C)cc2c1-c1cccnc1[C@H](C)C(=O)N2CC(=O)Nc1ccc(C(F)(F)F)cc1]

Example #162 was obtained during the preparation of Example #161. Purification by reverse phase chromatography (with basic elution) afforded the title product (8 mg, yield: 7%) as a white solid. LC-MS (Method B4) m/z [M+H]⁺: 515.2; rt: 4.43 min; purity>99%. LC-MS (Method A4) m/z [M+H]⁺: 515.2; rt: 4.37 min; purity: 99%. ¹H NMR (400 MHz, DMSO-d₆) δ 10.56 (s, 1H), 8.58 (dd, J=4.8, 1.8 Hz, 1H), 8.20 (dd, J=8.0, 1.8 Hz, 1H), 7.77 (d, J=8.8 Hz, 2H), 7.67 (d, J=8.8 Hz, 2H), 7.42 (dd, J=8.0, 4.8 Hz, 1H), 7.02 (s, 1H), 4.62 (ddd, J=11.9, 6.3, 3.3 Hz, 1H), 4.52 (s, 2H), 4.30 (ddd, J=11.9, 5.9, 3.3 Hz, 1H), 3.73-3.59 (m, 1H), 3.28 (s, 3H), 2.45 (s, 3H), 1.48 (d, J=6.7 Hz, 3H).

### Example #163: 2-(15-chloro-8,13-dimethyl-9-oxo-4,6,10,14-tetrazatricyclo[9.4.0.0^{2,7}]pentadeca-1(11),2(7),3,5,12,14-hexaen-10-yl)-N-[4-(trifluoromethyl)phenyl]acetamide

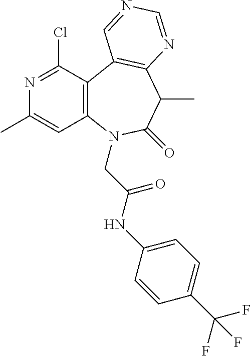 [SMILES: Cc1cc2c(c(Cl)n1)-c1cncnc1C(C)C(=O)N2CC(=O)Nc1ccc(C(F)(F)F)cc1]

A solution of Example #112 (166 mg, 0.347 mmol) in a 4 M solution of HCl in 1,4-dioxane (3.0 mL) was heated at 100° C. in a sealed vial for 1.5 h. The reaction mixture was cooled to room temperature then poured into saturated aqueous sodium bicarbonate (50 mL) and extracted with ethyl acetate (3×20 mL). The organic layers were combined, dried over sodium sulfate and concentrated under vacuum. The residue was purified by flash chromatography on silica (using a gradient of 0-100% EtOAc/iso-hexane as eluent) to afford the title compound (91 mg, yield: 53%) as a colorless solid. LC-MS (Method A7) m/z [M+H]⁺: 476/478; rt: 2.16 min; purity: 97%. ¹H NMR (400 MHz, DMSO-d₆) δ 10.58 (s, 1H), 9.24 (s, 1H), 9.23 (s, 1H), 7.74 (d, J=8.6 Hz, 2H), 7.67 (d, J=8.6 Hz, 2H), 7.50 (s, 1H), 4.64 (d, J=16.9 Hz, 1H), 4.55 (d, J=16.9 Hz, 1H), 3.96 (q, J=6.6 Hz, 1H), 2.55 (s, 3H), 1.47 (d, J=6.6 Hz, 3H).

### Example #164: 2-(1-chloro-10-methoxy-3,7-dimethyl-6-oxo-7H-pyrido[4,3-d][3]benzazepin-5-yl)-N-[4-(trifluoromethyl)phenyl]acetamide

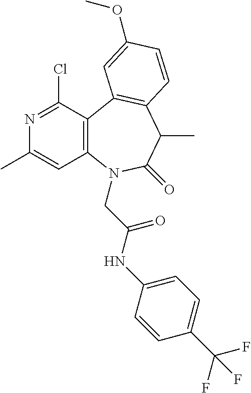 [SMILES: COc1ccc2c(c1)-c1c(cc(C)nc1Cl)N(CC(=O)Nc1ccc(C(F)(F)F)cc1)C(=O)C2C]

The title product was prepared according to general procedure 1, starting from Intermediate N44 and 2-chloro-N-(4-trifluoromethyl-phenyl)acetamide. Purification by column chromatography on silica gel (using a gradient of 0-100% EtOAc/iso-hexane as eluent) afforded the title compound (23 mg, yield: 54%) as a white solid. LC-MS (Method A7) m/z [M+H]⁺: 504/506; rt: 2.50 min; purity 98%. ¹H NMR (400 MHz, DMSO-d₆) δ 10.57 (s, 1H), 7.78 (d, J=8.6 Hz, 2H), 7.68 (d, J=8.6 Hz, 2H), 7.38 (s, 1H), 7.33-7.22 (m, 2H), 7.10 (dd, J=8.7, 2.7 Hz, 1H), 4.50 (s, 2H), 3.80 (s, 3H), 3.48 (q, J=6.7 Hz, 1H), 2.51 (s, 3H), 1.42 (d, J=6.7 Hz, 3H).

### Example #165: 2-[1-fluoro-8-(hydroxymethyl)-3-methyl-6-oxo-7H-pyrido[4,3-d][3]benzazepin-5-yl]-N-[4-(trifluoromethyl)phenyl]acetamide

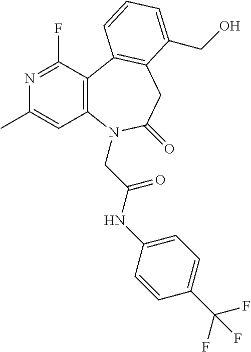 [SMILES: Cc1cc2c(c(F)n1)-c1cccc(CO)c1CC(=O)N2CC(=O)Nc1ccc(C(F)(F)F)cc1]

The title product was prepared according to general procedure 1, starting from Intermediate N45 and 2-chloro-N-(4-trifluoromethyl-phenyl)acetamide. Purification by flash chromatography on silica gel (using a gradient of 0-10% methanolic ammonia (0.7 M)/DCM as eluent) afforded the title compound (21 mg, yield: 23%) as an off white solid. LC-MS (Method A7) m/z: [M+H]⁺: 474; rt: 2.11 min; purity: 97%. ¹H NMR (400 MHz, DMSO-d₆) δ 10.60 (s, 1H), 7.78 (d, J=8.5 Hz, 2H), 7.68 (d, J=8.5 Hz, 2H), 7.61-7.50 (m, 2H), 7.40 (t, J=7.7 Hz, 1H), 7.31 (s, 1H), 5.32 (dd, J=5.9, 4.7 Hz, 1H), 4.78 (dd, J=13.3, 5.9 Hz, 1H), 4.63-4.57 (m, 1H), 4.55 (d, J=6.6 Hz, 2H), 3.90 (d, J=12.9 Hz, 1H), 3.29 (s, 1H), 2.48 (s, 3H).

### Example #166: N-(4-chloro-3-fluoro-phenyl)-2-(1,3,7-trimethyl-2,6-dioxo-pyrido[3,2-d][1,3]benzodiazepin-5-yl)acetamide

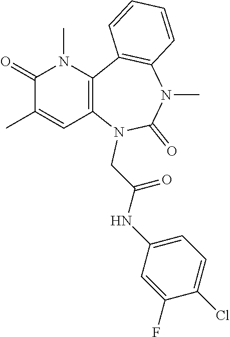 [SMILES: Cc1cc2c(n(C)c1=O)-c1ccccc1N(C)C(=O)N2CC(=O)Nc1ccc(Cl)c(F)c1]

To a solution of 2-(1,3,7-trimethyl-2,6-dioxo-pyrido[3,2-d][1,3]benzodiazepin-5-yl)acetic acid (Intermediate N67, 80 mg, 0.232 mmol) in dry DMF (3.0 mL), was added HATU (185 mg, 0.488 mmol), N,N-diisopropylethylamine (0.12 mL, 0.697 mmol) and 4-chloro-3-fluoro-aniline (43 mg, 0.293 mmol) and the reaction mixture was stirred at room temperature for 16 h. The reaction mixture was diluted with water (15 mL) and extracted with EtOAc (3×20 mL). The combined organic extracts were washed with aq. LiCl (1 M, 20 mL) and brine (20 mL), dried over MgSO₄, filtered and concentrated under vacuum. The residue was purified by preparative HPLC (Waters, Acidic (0.1% Formic acid), Acidic, Waters X-Select Prep-C18, 5 μm, 19×50 mm column, 5-100% MeCN in Water) to afford the title compound (20 mg, yield: 18%) as a white solid. LC-MS (Method A7) m/z [M+H]⁺: 455.2; rt: 2.03 min; purity: 100%. ¹H NMR (400 MHz, DMSO-d₆) δ 10.32 (s, 1H), 7.69-7.58 (m, 2H), 7.58-7.51 (m, 2H), 7.48 (t, J=8.7 Hz, 1H), 7.41 (dd, J=8.4, 1.1 Hz, 1H), 7.36-7.30 (m, 1H), 7.24 (dt, J=9.0, 1.6 Hz, 1H), 4.40 (d, J=16.5 Hz, 1H), 4.11 (d, J=16.5 Hz, 1H), 3.36 (s, 3H), 3.08 (s, 3H), 2.08 (d, J=1.1 Hz, 3H).

### Example #167: 2-(5-cyano-3-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

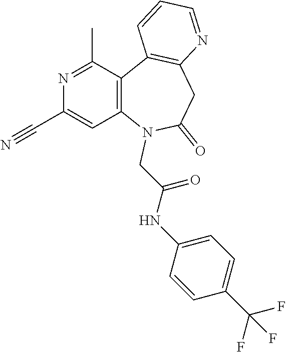 [SMILES: Cc1nc(C#N)cc2c1-c1cccnc1CC(=O)N2CC(=O)Nc1ccc(C(F)(F)F)cc1]

The title product was prepared according to general procedure 1, starting from Intermediate N53 and 2-chloro-N-[4-(trifluoromethyl)phenyl]acetamide (CAS 2707-23-5). Purification by trituration with MTBE afforded the title compound (2.26 mg, yield: 7%) as a white solid. LC-MS (Method A7) m/z [M+H]⁺: 452.2; rt: 2.05 min, purity: >99%. ¹H NMR (400 MHz, DMSO-d₆) δ 10.61 (s, 1H), 8.65 (dd, J=4.9, 1.7 Hz, 1H), 8.23 (dd, J=8.0, 1.7 Hz, 1H), 8.07 (s, 1H), 7.76 (d, J=8.6 Hz, 2H), 7.68 (d, J=8.6 Hz, 2H), 7.52 (dd, J=7.9, 4.8 Hz, 1H), 4.69 (d, J=17.0 Hz, 1H), 4.57 (d, J=17.0 Hz, 1H), 3.89 (d, J=12.6 Hz, 1H), 3.69 (d, J=12.6 Hz, 1H), 2.60 (s, 3H).

### Example #168: 2-(3,5-dichloro-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

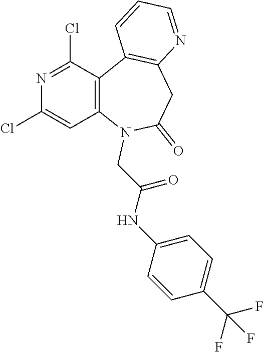 [SMILES: O=C(CN1C(=O)Cc2ncccc2-c2c1cc(Cl)nc2Cl)Nc1ccc(C(F)(F)F)cc1]

The title product was prepared according to general procedure 1, starting from Intermediate N54 and 2-chloro-N-[4-(trifluoromethyl)phenyl]acetamide (CAS 2707-23-5). Purification by flash chromatography on silica gel (using a gradient of 40 to 100% EtOAc in iso-hexane as eluent) afforded the title compound (96 mg, yield: 83%) as a white solid. LC-MS (Method A7) m/z [M+H]⁺: 481.2/483.2; rt: 2.25 min, purity: 97%. ¹H NMR (400 MHz, DMSO-d₆) δ 10.62 (s, 1H), 8.64 (dd, J=4.9, 1.7 Hz, 1H), 8.29 (dd, J=8.0, 1.7 Hz, 1H), 7.84-7.63 (m, 5H), 7.53 (dd, J=8.0, 4.9 Hz, 1H), 4.69 (d, J=16.9 Hz, 1H), 4.56 (d, J=16.9 Hz, 1H), 4.05 (d, J=12.7 Hz, 1H), 3.68 (d, J=12.7 Hz, 1H).

### Example #169: 2-[3-chloro-5-(methylamino)-9-oxo-4,8,12-triazatricyclo[9.4.0.0^{2,7}]pentadeca-1(11),2(7),3,5,12,14-hexaen-8-yl]-N-[4-(trifluoromethyl)phenyl]acetamide

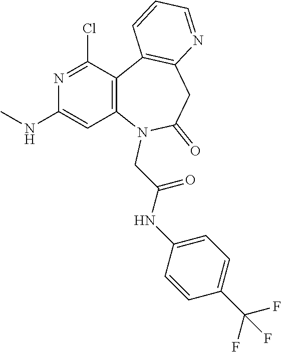 [SMILES: CNc1cc2c(c(Cl)n1)-c1cccnc1CC(=O)N2CC(=O)Nc1ccc(C(F)(F)F)cc1]

A solution of Example #168 (32 mg, 0.066 mmol) and a 2M solution of methylamine in THF (0.2 mL, 0.395 mmol) in DMF (0.75 mL) was heated to 80° C. for 18 h. Water (25 mL) was added and the aqueous phase was extracted with EtOAc (3×25 mL). The combined organics were washed with brine (3×25 mL), dried over magnesium sulfate, filtered and concentrated under vacuum. The residue was purified by preparative HPLC (Waters, (0.1% Formic acid), Acidic, Waters X-Select Prep-C18, 5 μm, 19×50 mm column, 5-95% MeCN in Water) to afford the title compound (10 mg, yield: 19%) as an off-white solid. LC-MS (Method A7) m/z [M+H]⁺: 476.2/478.2; rt: 2.08 min, purity: 100%. ¹H NMR (400 MHz, CD30D) δ 8.46 (dd, J=4.9, 1.6 Hz, 1H), 8.23 (dd, J=8.0, 1.6 Hz, 1H), 7.76 (d, J=8.6 Hz, 2H), 7.61 (d, J=8.6 Hz, 2H), 7.44 (dd, J=8.0, 4.9 Hz, 1H), 6.57 (s, 1H), 4.58 (d, J=16.7 Hz, 1H), 4.35 (d, J=16.7 Hz, 1H), 3.94 (d, J=12.5 Hz, 1H), 3.76 (d, J=12.5 Hz, 1H), 2.90 (s, 3H). 2 NHs not observed.

### Example #170: 2-[3-chloro-5-(1-methylazetidin-3-yl)-9-oxo-4,8,12-triazatricyclo[9.4.0.0^{2,7}]pentadeca-1(11),2(7),3,5,12,14-hexaen-8-yl]-N-[4-(trifluoromethyl)phenyl]acetamide

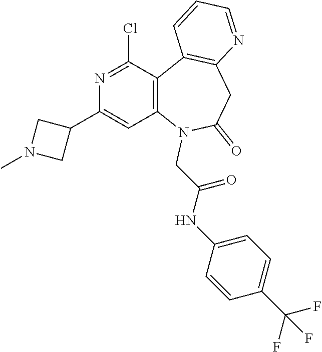 [SMILES: CN1CC(c2cc3c(c(Cl)n2)-c2cccnc2CC(=O)N3CC(=O)Nc2ccc(C(F)(F)F)cc2)C1]

### Step 1: Synthesis of tert-butyl 3-[3-chloro-9-oxo-8-[2-oxo-2-[4-(trifluoromethyl)anilino]ethyl]-4,8,12-triazatricyclo[9.4.0.0^{2,7}]pentadeca-1(11),2,4,6,12,14-hexaen-5-yl]azetidine-1-carboxylate #170_1

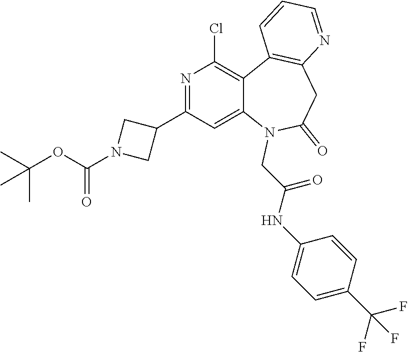 [SMILES: CC(C)(C)OC(=O)N1CC(c2cc3c(c(Cl)n2)-c2cccnc2CC(=O)N3CC(=O)Nc2ccc(C(F)(F)F)cc2)C1]

A solution of Example #168 (40 mg, 0.08 mmol), cuprous iodide (0.6 mg, 0.003 mmol) and [1,1′-bis(diphenylphosphino)ferrocene]dichloropalladium(II) (5 mg, 0.006 mmol) in dry DMA (0.5 mL) was degassed with nitrogen for 5 min. The reaction mixture was then heated at 85° C. under inert atmosphere. (1-tert-Butoxycarbonylazetidin-3-yl)-iodo-zinc (450 mmol/L, 0.8 mL, 0.361 mmol) was added and the reaction mixture was stirred for 30 min, always at the same temperature. Additional (1-tert-butoxycarbonylazetidin-3-yl)-iodo-zinc (450 mmol/L, 0.16 mL, 0.071 mmol) was added and the reaction mixture was stirred for a further 30 min at 85° C. The reaction mixture was treated with saturated aq. NH₄Cl and extracted with EtOAc (3×50 mL). The combined organic extracts were dried over magnesium sulfate, filtered and concentrated under vacuum. The residue was dry loaded onto silica and purified by flash chromatography on silica gel (using a gradient of 50 to 100% EtOAc in iso-hexane as eluent) to afford the title compound (20 mg, yield: 37%) as a beige solid. LC-MS (Method A7) m/z [M+H]⁺: 602.2/604.2; rt: 2.44 min, purity: 90%.

### Step 2: Synthesis of 2-[5-(azetidin-3-yl)-3-chloro-9-oxo-4,8,12-triazatricyclo[9.4.0.0^{2,7}]pentadeca-1(11),2,4,6,12,14-hexaen-8-yl]-N-[4-(trifluoromethyl)phenyl]acetamide #170_2

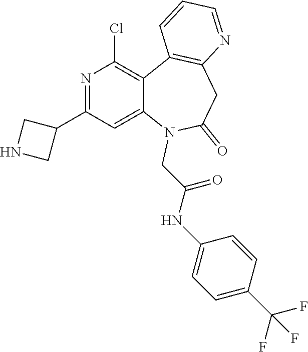 [SMILES: O=C(CN1C(=O)Cc2ncccc2-c2c1cc(C1CNC1)nc2Cl)Nc1ccc(C(F)(F)F)cc1]

Tert-Butyl 3-[3-chloro-9-oxo-8-[2-oxo-2-[4-(trifluoromethyl)anilino]ethyl]-4,8,12-triazatricyclo[9.4.0.0^{2,7}]pentadeca-1(11),2,4,6,12,14-hexaen-5-yl]azetidine-1-carboxylate (#170_1, 51 mg, 0.077 mmol) was dissolved in DCM (1.3 mL) and a 4N solution of HCl in 1,4-dioxane (1.3 mL, 5.20 mmol) was added and the reaction mixture was stirred for 1 h at room temperature. The reaction mixture was concentrated under vacuum, dissolved in MeOH (5 mL) and loaded onto an SCX plug (0.5 g). The plug was washed with MeOH (3×15 mL) and the product was eluted with NH₃ in MeOH (0.7 M; 3×15 mL). The filtrate was concentrated under vacuum to afford the title compound (38 mg, yield: 83%) as an off-white solid. LC-MS (Method A7) m/z [M+H]⁺: 502.2/504.2; rt: 1.41 min, purity: 85%.

### Step 3: Synthesis of 2-[3-chloro-5-(1-methylazetidin-3-yl)-9-oxo-4,8,12-triazatricyclo[9.4.0.0^{2,7}]pentadeca-1(11),2(7),3,5,12,14-hexaen-8-yl]-N-[4-(trifluoromethyl)phenyl]acetamide #170

A stirred solution of 2-[5-(azetidin-3-yl)-3-chloro-9-oxo-4,8,12-triazatricyclo[9.4.0.0^{2,7}]pentadeca-1(11),2,4,6,12,14-hexaen-8-yl]-N-[4-(trifluoromethyl)phenyl]acetamide (#170_2, 38 mg, 0.064 mmol) in DCE (3 mL) and THF (1 mL) was treated with a 37% aqueous solution of formaldehyde (0.05 mL, 0.637 mmol) and stirred for 10 min. Sodium triacetoxyborohydride (54 mg, 0.255 mmol) was added and the reaction mixture was stirred for 16 h at room temperature. The volatiles were removed under vacuum and the residue was partitioned between EtOAc (30 mL) and water (30 mL). The layers were separated and the organic layer discarded. The aqueous layer was basified (~pH 8) with 2M aq. NaOH. The basic aqueous layer was extracted with EtOAc (3×30 mL). The combined organic layers were dried over sodium sulfate, filtered and concentrated under vacuum. The compound was purified by reversed phase preparative HPLC (Waters XBridge BEH C18 ODB prep column, eluting 30-60% acetonitrile in water with 0.3% ammonia) to afford the title compound (3 mg, yield: 9%) as a white solid. LC-MS (Method A7) m/z [M+H]⁺: 516.2/518.2; rt: 1.45 min, purity: 96%. ¹H NMR (400 MHz, DMSO-d₆) δ 10.59 (s, 1H), 8.61 (dd, J=4.8, 1.7 Hz, 1H), 8.27 (dd, J=8.0, 1.7 Hz, 1H), 7.80-7.61 (m, 4H), 7.55-7.45 (m, 2H), 4.71-4.48 (m, 2H), 3.93 (d, J=12.6 Hz, 1H), 3.76 (p, J=7.4 Hz, 1H), 3.67 (d, J=12.6 Hz, 1H), 3.63-3.54 (m, 2H), 3.24 (m, 2H), 2.24 (s, 3H).

### Example #171: 2-(3,12-difluoro-5,10-dimethyl-9-oxo-4,8,14-triazatricyclo[9.4.0.0^{2,7}]pentadeca-1(11),2(7),3,5,12,14-hexaen-8-yl)-N-[4-(trifluoromethyl)phenyl]acetamide

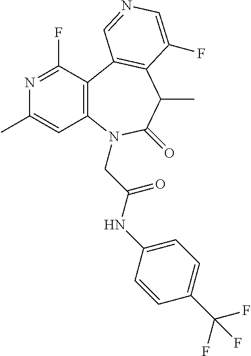 [SMILES: Cc1cc2c(c(F)n1)-c1cncc(F)c1C(C)C(=O)N2CC(=O)Nc1ccc(C(F)(F)F)cc1]

The title product was prepared according to general procedure 1, starting from Intermediate N55 and 2-chloro-N-[4-(trifluoromethyl)phenyl]acetamide (CAS 2707-23-5). Purification by flash chromatography on silica gel (using a gradient of 0 to 10% MeOH in DCM) afforded the title compound (56 mg, yield: 65%) as a white solid. LC-MS (Method A7) m/z [M+H]⁺: 477.2; rt: 2.28 min; purity: 98%. ¹H NMR (400 MHz, DMSO-d₆) δ 10.66 (s, 0.2H), δ 10.61 (s, 0.8H), 8.80-8.78 (m, 0.2H) 8.74-8.69 (m, 1H), 8.64-8.62 (m, 0.8H), 7.78 (m, 2H), 7.67 (m, 2H), 7.38 (s, 0.8H), 7.35 (s, 0.2H), 4.71-4.54 (m, 2H), 4.51 (m, 0.2H) 3.82 (q, J=6.8 Hz, 0.8H), 1.61 (t, J=6.4 Hz, 2.2H), 1.06 (d, J=7.5 Hz, 0.8H). One CH₃ obscured by solvent peak. Presence of diastereoisomers in a ratio of −1:4. ¹⁹F NMR (376 MHz, DMSO-d₆) δ−60.32 (d, J=7.7 Hz), −70.00, −70.09, −133.97, −136.26

### Example #172: 2-(3-fluoro-5,10-dimethyl-9-oxo-4,8,12,13-tetrazatricyclo[9.4.0.0^{2,7}]pentadeca-1(11),2(7),3,5,12,14-hexaen-8-yl)-N-[4-(trifluoromethyl)phenyl]acetamide

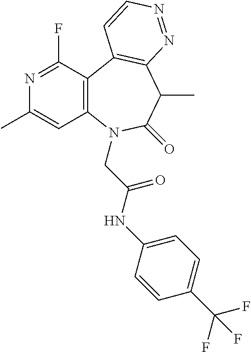 [SMILES: Cc1cc2c(c(F)n1)-c1ccnnc1C(C)C(=O)N2CC(=O)Nc1ccc(C(F)(F)F)cc1]

The title product was prepared according to general procedure 1, starting from Intermediate N56 and 2-chloro-N-[4-(trifluoromethyl)phenyl]acetamide (CAS 2707-23-5). Purification by flash chromatography on silica gel (using a gradient of 0 to 10% MeOH in DCM as eluent) afforded the title compound (272 mg, yield: 56%) as a white solid. LC-MS (Method A7) m/z [M+H]⁺: 460.2; rt: 2.04 min; purity: 98%. ¹H NMR (400 MHz, DMSO-d₆) δ 10.61 (s, 1H), 9.36 (d, J=5.3 Hz, 1H), 8.03 (dd, J=5.3, 4.2 Hz, 1H), 7.78-7.63 (m, 4H), 7.43 (s, 1H), 4.69-4.57 (m, 2H), 4.09 (q, J=6.6 Hz, 1H), 2.52 (s, 3H), 1.63 (d, J=6.6 Hz, 3H).

### Example #173: 2-(3-chloro-5,10-dimethyl-9-oxo-4,8,12,13-tetrazatricyclo[9.4.0.0^{2,7}]pentadeca-1(11),2(7),3,5,12,14-hexaen-8-yl)-N-[4-(trifluoromethyl)phenyl]acetamide

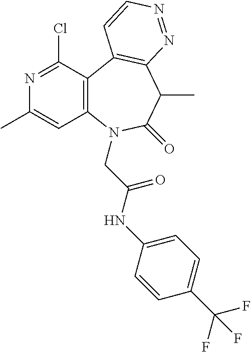 [SMILES: Cc1cc2c(c(Cl)n1)-c1ccnnc1C(C)C(=O)N2CC(=O)Nc1ccc(C(F)(F)F)cc1]

A mixture of Example #172 (180 mg, 0.392 mmol) in a 4N solution of HCl in 1,4-dioxane (5.0 mL, 20.0 mmol) was heated at 100° C. in a sealed microwave vial for 1 h. The reaction mixture was allowed to cool to room temperature and poured onto saturated aqueous sodium bicarbonate solution (100 mL). The product was extracted with EtOAc (2×40 mL) and the combined organic extracts were dried over sodium sulfate, filtered and concentrated under vacuum. The residue was purified by flash chromatography on silica gel (using a gradient of 0 to 10% MeOH in DCM as eluent) to give the title compound (102 mg, yield: 54%) as a white solid. LC-MS (Method A7) m/z [M+H]⁺: 476.1/478.2; rt: 2.06 min; purity: 98%. ¹H NMR (400 MHz, DMSO-d₆) δ 10.58 (s, 1H), 9.37 (d, J=5.3 Hz, 1H), 8.16 (d, J=5.3 Hz, 1H), 7.73 (d, J=8.6 Hz, 2H), 7.66 (d, J=8.6 Hz, 2H), 7.51 (s, 1H), 4.66 (d, J=16.8 Hz, 1H), 4.54 (d, J=16.8 Hz, 1H), 4.12 (q, J=6.6 Hz, 1H), 2.54 (d, J=2.6 Hz, 3H), 1.62 (d, J=6.6 Hz, 3H).

### Example #174: Enantiomer (7R) or (7S) of N-(4-chloro-3-fluoro-phenyl)-2-(9-fluoro-2,3,7-trimethyl-1,6-dioxo-7H-pyrido[4,3-d][3]benzazepin-5-yl)acetamide

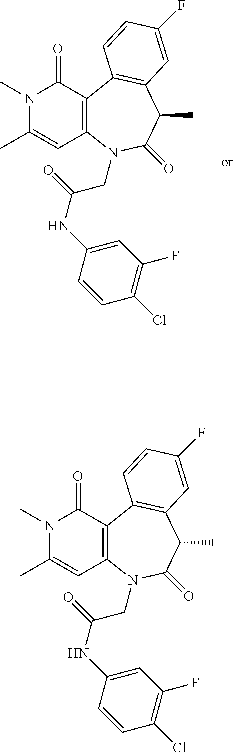

[2-(9-fluoro-2,3,7-trimethyl-1,6-dioxo-7H-pyrido[4,3-d][3]benzazepin-5-yl)acetyl]oxylithium (Intermediate N70, 125 mg, 0.339 mmol) was dissolved in dry DMF (15.0 mL) and 4-chloro-3-fluoro-aniline (99 mg, 0.678 mmol), N,N-diisopropylethylamine (0.25 mL, 1.38 mmol) and HATU (387 mg, 1.02 mmol) were sequentially added. The reaction mixture was stirred at room temperature for 16 h. The reaction mixture was diluted with EtOAc (130 mL) and washed with water (2×80 mL) and brine (2×80 mL). The organic phase was dried over sodium sulfate, filtered and concentrated under vacuum. The residue was purified by flash chromatography on silica gel (using a gradient of 0 to 100% EtOH:EtOAc (1:3) in iso-hexane as eluent) to afford the title compound (131 mg, yield: 78%) as an off-white solid. The racemate was separated by chiral chromatography (SFC Chiralcel OD, CO₂+Ethanol 20%). Chiral purity: 99%; rt=1.92 min (second eluting enantiomer). For information, first eluting enantiomer rt=1.55 min. Both measured by HPLC, Chiralcel OD from Daicel, Solvent: ACN 100%—DEA 0.1%). LC-MS (Method A7) m/z [M+H]⁺: 472.2/474.2; rt: 2.17 min; purity: 99%. ¹H NMR (400 MHz, DMSO-d₆) δ 10.51 (s, 1H), 7.88 (dd, J=8.8, 6.1 Hz, 1H), 7.74 (dd, J=11.8, 2.3 Hz, 1H), 7.52 (t, J=8.8 Hz, 1H), 7.34-7.27 (m, 1H), 7.17 (td, J=8.6, 2.7 Hz, 1H), 7.08 (dd, J=10.2, 2.7 Hz, 1H), 6.33 (s, 1H), 4.38 (s, 2H), 3.50 (s, 3H), 3.43 (d, J=6.8 Hz, 1H), 2.42 (s, 3H), 1.43 (d, J=6.8 Hz, 3H).

### Example #175: Enantiomer (7R) or (7S) of N-[4-(difluoromethyl)phenyl]-2-(2,3,7-trimethyl-1,6-dioxo-7H-pyrido[4,3-d][3]benzazepin-5-yl)acetamide

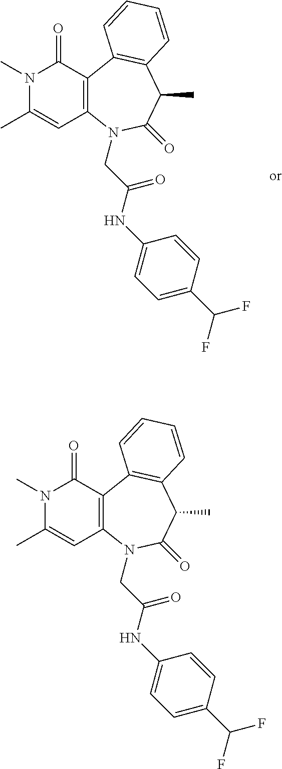 [SMILES: Cc1cc2c(c(=O)n1C)-c1ccccc1[C@@H](C)C(=O)N2CC(=O)Nc1ccc(C(F)F)cc1 ; Cc1cc2c(c(=O)n1C)-c1ccccc1[C@H](C)C(=O)N2CC(=O)Nc1ccc(C(F)F)cc1]

The title product was prepared according to a similar procedure as the one depicted for example #174 but starting from Intermediate N109 and 4-(difluoromethyl)aniline. The racemate was purified by chiral chromatography (SFC Chiralpak OD-I, CO₂+MeOH 20%). Chiral purity: 99%; rt=2.29 min (second eluting enantiomer). For information, first eluting enantiomer rt=1.79 min. Both measured by HPLC, Chiralpak IB from Daicel, ACN 100%—DEA 0.1%). LCMS (Method A7): m/z [M+H]⁺: 452.2; rt: 1.95 min; purity: 100%. ¹H NMR (400 MHz, DMSO-d₆) δ 10.38 (s, 1H), 7.84 (dd, J=7.9, 1.4 Hz, 1H), 7.70 (d, J=8.4 Hz, 2H), 7.52 (d, J=8.4 Hz, 2H), 7.40 (td, J=7.6, 1.4 Hz, 1H), 7.35-7.24 (m, 2H), 6.97 (t, J=56.2 Hz, 1H), 6.34 (s, 1H), 4.43-4.33 (m, 2H), 3.50 (s, 3H), 3.41 (q, J=6.7 Hz, 1H), 2.42 (s, 3H), 1.45 (d, J=6.7 Hz, 3H).

Examples #176, #177 and #178 were obtained by chiral separation of the corresponding racemate by chiral chromatography, (SFC Chiralpak IA, CO₂+MeOH 25%, SFC Chiralpak OD-I, CO₂+MeOH 20% and HPLC Whelk O1 (R,R)—Ethanol 100%, respectively).

| Ex. | Structure Example | Example Name | Protocolumn and characterization |
|---|---|---|---|
| #176 | 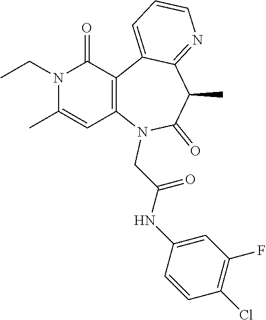 [SMILES: CCn1c(C)cc2c(c1=O)-c1cccnc1[C@@H](C)C(=O)N2CC(=O)Nc1ccc(Cl)c(F)c1] or | Enantiomer (10R) or (10S) of N-(4-chloro-3- fluoro-phenyl)-2-[4- ethyl-5,10-dimethyl- 3,9-dioxo-4,8,12- triazatricyclo[9.4.0.0^{2,7}] pentadeca- 1(15),2(7),5,11,13- pentaen-8- yl]acetamide | Prepared from Intermediate N110 and 4-chloro-3-fluoro-aniline. LCMS (Method A7: m/z [M + H]⁺: 469.2/471.2; rt: 1.91 min; purity: 99%. ¹H NMR (400 MHz, DMSO-d₆) δ 10.52 (s, 1H), 8.54 (dd, J = 4.7, 1.8 Hz, 1H), 8.25 (dd, J = 8.0, 1.8 Hz, 1H), 7.74 (dd, J = 11.9, 2.4 Hz, 1H), 7.52 (t, J = 8.7 Hz, 1H), 7.38 (dd, J = 8.0, 4.7 Hz, 1H), 7.30 (ddd, J = 8.9, 2.5, 0.9 Hz, 1H), 6.36 (s, 1H), 4.48- 4.34 (m, 2H), 4.07 (q, J = 7.0 Hz, 2H), 3.59 (q, J = 6.6 Hz, 1H), 2.47 (s, 3H), 1.47 (d, J = 6.6 Hz, 3H), 1.24 (t, J = 7.0 Hz, 3H). Chiral purity: >99%; rt = 3.87 min (second eluting enantiomer). For information, first eluting enantiomer rt = 1.91 min. Both measured by HPLC, Chiralpak IB from Daicel, ACN 100 % - DEA 0.1%) |
| | 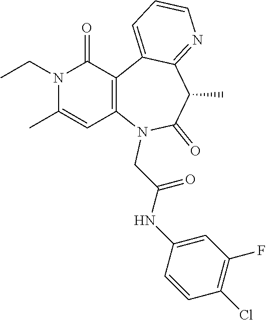 [SMILES: CCn1c(C)cc2c(c1=O)-c1cccnc1[C@H](C)C(=O)N2CC(=O)Nc1ccc(Cl)c(F)c1] | | |
| #177 | 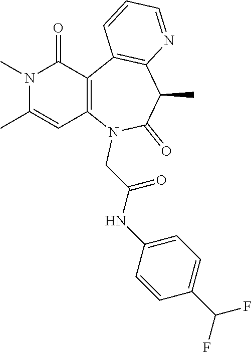 [SMILES: Cc1cc2c(c(=O)n1C)-c1cccnc1[C@@H](C)C(=O)N2CC(=O)Nc1ccc(C(F)F)cc1] | Enantiomer (10R) or (10S) of N-[4- (difluoromethyl)phenyl]- 2-[4,5,10-trimethyl- 3,9-dioxo-4,8,12- triazatricyclo[9.4.0.0^{2,7}] pentadeca- 1(11),2(7),5,12,14- pentaen-8- yl]acetamide | Prepared from Intermediate N111 and 4-(difluoromethyl)aniline. LCMS (Method A7: m/z [M + H]⁺ 453.2; rt: 1.58 min; purity: 96%. ¹H NMR (400 MHz, DMSO-d₆) δ 10.40 (s, 1H), 8.55 (dd, J = 4.7, 1.7 Hz, 1H), 8.24 (dd, J = 7.9, 1.7 Hz, 1H), 7.69 (d, J = 8.4 Hz, 2H), 7.54-7.47 (m, 2H), 7.38 (dd, J = 7.9, 4.7 Hz, 1H), 6.96 (t, J = 56.1 Hz, 1H), 6.40 (d, J = 0.9 Hz, 1H), 4.44 (s, 2H), 3.59 (d, J = 6.7 Hz, 1H), 3.51 (s, 3H), 2.43 (s, 3H), 1.48 (d, J = 6.7 Hz, 3H). Chiral purity: 98%; rt = 1.81 min (first eluting enantiomer). For information, second eluting enantiomer rt = 3.66 min. Both measured by HPLC, Whelk O-1 (R,R) from Regis Technology (5μ) ACN 100% - DEA 0.1%) |
| | or | | |
| | 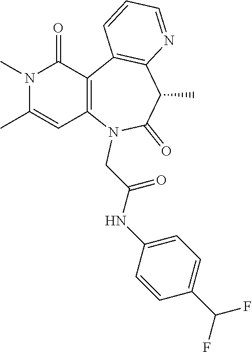 [SMILES: Cc1cc2c(c(=O)n1C)-c1cccnc1[C@H](C)C(=O)N2CC(=O)Nc1ccc(C(F)F)cc1] | | |
| #178 | 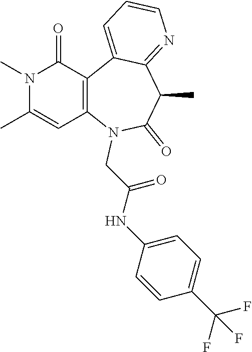 [SMILES: Cc1cc2c(c(=O)n1C)-c1cccnc1[C@@H](C)C(=O)N2CC(=O)Nc1ccc(C(F)(F)F)cc1] | Enantiomer (10R) or (10S) of N-[4- (trifluoromethyl)phenyl]- 2-[4,5,10-trimethyl- 3,9-dioxo-4,8,12- triazatricyclo[9.4.0.0^{2,7}] pentadeca- 1(15),2(7),5,11,13- pentaen-8- yl]acetamide | Prepared from Intermediate N111 and 4-(trifluoromethyl)aniline. LCMS (Method B5): m/z [M + H]⁺: 471.2; rt: 1.85 min; purity >99%. ¹H NMR (400 MHz, DMSO-d₆) δ 10.55 (s, 1H), 8.55 (dd, J = 4.7, 1.8 Hz, 1H), 8.24 (dd, J = 7.9, 1.8 Hz, 1H), 7.76 (d, J = 8.5 Hz, 2H), 7.67 (d, J = 8.5 Hz, 2H), 7.38 (dd, J = 7.9, 4.7 Hz, 1H), 6.40 (s, 1H), 4.52-4.40 (m, 2H), 3.59 (q, J = 6.6 Hz, 1H), 3.51 (s, 3H), 2.43 (s, 3H), 1.48 (d, J = 6.6 Hz, 3H). Chiral purity: >99%; rt = 4.28 min (second eluting enantiomer). For information, first eluting enantiomer, rt = 2.18 min. Both measured by HPLC, Whelk O-1 (R,R) from Regis Technology (3μ): Solvent: EtOH 100% = DEA 0.1%) |
| | or | | |
| | 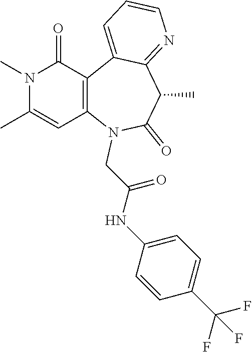 [SMILES: Cc1cc2c(c(=O)n1C)-c1cccnc1[C@H](C)C(=O)N2CC(=O)Nc1ccc(C(F)(F)F)cc1] | | |

### Example #179: N-(4-chloro-3-fluoro-phenyl)-2-(4-cyclopropyl-5,10-dimethyl-3,9-dioxo-4,8,12-triazatricyclo[9.4.0.0^{2,7}]pentadeca-1(11),2(7),5,12,14-pentaen-8-yl)acetamide

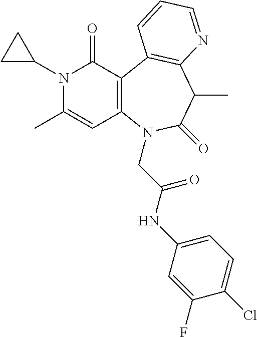 [SMILES: Cc1cc2c(c(=O)n1C1CC1)-c1cccnc1C(C)C(=O)N2CC(=O)Nc1ccc(Cl)c(F)c1]

[2-(4-Cyclopropyl-5,10-dimethyl-3,9-dioxo-4,8,12-triazatricyclo[9.4.0.0^{2,7}]pentadeca-1(11),2(7),5,12,14-pentaen-8-yl)acetyl]oxylithium (Intermediate N71, 92.0 mg, 0.256 mmol) was dissolved in dry DMF (15.0 mL) and 4-chloro-3-fluoro-aniline (74 mg, 0.510 mmol), N,N-diisopropylethylamine (0.17 mL, 1.02 mmol) and HATU (292 mg, 0.768 mmol) were sequentially added. The reaction mixture was stirred at room temperature for 24 h. The reaction mixture was diluted with EtOAc (130 mL) and washed with water (2×80 mL) and brine (2×80 mL). The organic phase was dried over sodium sulfate, filtered, and concentrated under vacuum. The residue was purified by flash chromatography on silica gel (using a gradient of 0 to 100% EtOH:EtOAc (1:3) in iso-hexane as eluent) to afford the title compound (63 mg, yield: 50%) as an off-white solid. LC-MS (Method A7) m/z [M+H]⁺: 481.2/483.2; rt: 1.90 min; purity: 99%. ¹H NMR (400 MHz, DMSO-d₆) δ 10.52 (s, 1H), 8.54 (dd, J=4.7, 1.8 Hz, 1H), 8.21 (dd, J=8.0, 1.8 Hz, 1H), 7.73 (dd, J=11.9, 2.4 Hz, 1H), 7.52 (t, J=8.7 Hz, 1H), 7.37 (dd, J=7.9, 4.7 Hz, 1H), 7.33-7.27 (m, 1H), 6.31 (s, 1H), 4.39 (s, 2H), 3.57 (q, J=6.7 Hz, 1H), 2.99-2.90 (m, 1H), 2.49 (s, 3H, DMSO overlap), 1.47 (d, J=6.7 Hz, 3H), 1.14 (t, J=6.7 Hz, 2H), 0.94-0.88 (m, 1H), 0.82-0.76 (m, 1H). ¹⁹F NMR (376 MHz, DMSO-d₆) δ−114.69.

### Example #180: 2-(3-fluoro-5,10-dimethyl-9-oxo-4,8,14-triazatricyclo[9.4.0.0^{2,7}]pentadeca-1(11),2(7),3,5,12,14-hexaen-8-yl)-N-[4-(trifluoromethyl)phenyl]acetamide

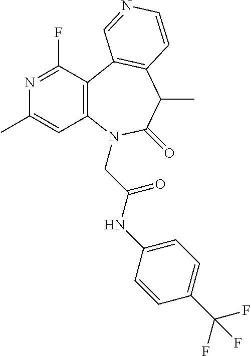 [SMILES: Cc1cc2c(c(F)n1)-c1cnccc1C(C)C(=O)N2CC(=O)Nc1ccc(C(F)(F)F)cc1]

The title product was prepared according to general procedure 1, starting from Intermediate N57 and 2-chloro-N-[4-(trifluoromethyl)phenyl]acetamide (CAS 2707-23-5). Purification by flash chromatography on silica gel (using a gradient of 0 to 10% MeOH in DCM as eluent) followed by reverse phase flash chromatography on C18 silica gel (using a gradient of 0 to 100% water in acetonitrile) afforded the title compound (13 mg, yield: 5%) as a white solid. LC-MS (Method A7) m/z [M+H]⁺: 459.2; rt: 1.97 min; purity: 95%. ¹H NMR (400 MHz, DMSO-d₆) δ 10.63 (s, 1H), 8.88-8.84 (m, 1H), 8.69-8.63 (m, 1H), 7.79-7.75 (m, 2H), 7.70-7.65 (m, 2H), 7.48-7.44 (m, 1H), 7.38 (s, 1H), 4.65-4.51 (m, 2H), 3.68 (q, J=6.7 Hz, 1H), 1.47 (d, J=6.7 Hz, 3H). 3H under DMSO peak.

### Example #181: N-(4-chloro-3-fluoro-phenyl)-2-(9-chloro-1,3,7-trimethyl-2,6-dioxo-7H-pyrido[3,2-d][3]benzazepin-5-yl)acetamide

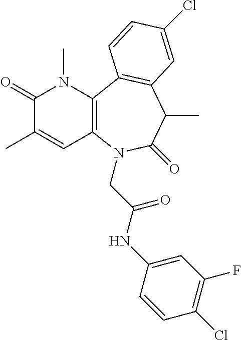 [SMILES: Cc1cc2c(n(C)c1=O)-c1ccc(Cl)cc1C(C)C(=O)N2CC(=O)Nc1ccc(Cl)c(F)c1]

To a solution of [2-(9-chloro-1,3,7-trimethyl-2,6-dioxo-7H-pyrido[3,2-d][3]benzazepin-5-yl)acetyl]oxylithium (Intermediate N73, 8.0 mg, 0.021 mmol) in dry DMF (0.5 mL), HATU (12 mg, 0.031 mmol) was added, followed by 4-chloro-3-fluoro-aniline (4.0 mg, 0.025 mmol) and then N,N-diisopropylethylamine (0.01 mL, 0.062 mmol). The reaction mixture was stirred at room temperature for 16 h. Additional 4-chloro-3-fluoro-aniline (2.0 mg, 0.012 mmol) was added and the reaction mixture was stirred at room temperature for a further 3 h. The reaction mixture was treated with water (20 mL) and extracted with EtOAc (3×50 mL). The combined organic extracts were washed with brine (3×50 mL), dried over magnesium sulfate, filtered and concentrated under vacuum. The residue was purified by reverse phase flash chromatography on C18 silica gel (using a gradient of 10 to 65% water in acetonitrile as eluent) to afford the title compound as a pale yellow powder (2.0 mg, yield: 18%). LC-MS (Method A7) m/z [M+H]⁺: 488.1/490.2/492.1; rt: 2.24 min; purity: 96%. ¹H NMR (400 MHz, CD30D) δ 7.73-7.61 (m, 3H), 7.50 (dd, J=8.4, 2.1 Hz, 1H), 7.45 (d, J=2.1 Hz, 1H), 7.40 (t, J=8.4 Hz, 1H), 7.24 (ddd, J=8.8, 2.4, 1.1 Hz, 1H), 4.38-4.25 (m, 2H), 3.79 (q, J=6.9 Hz, 1H), 3.52 (s, 3H), 2.25-2.21 (m, 3H), 1.59 (d, J=6.9 Hz, 3H). NH not observed.

### Example #182: 2-(3-fluoro-5-methoxy-10-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide

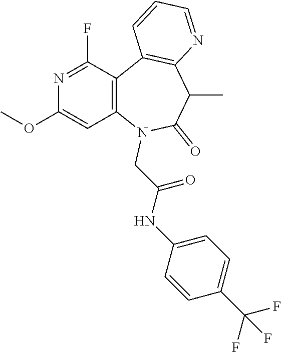 [SMILES: COc1cc2c(c(F)n1)-c1cccnc1C(C)C(=O)N2CC(=O)Nc1ccc(C(F)(F)F)cc1]

The title product was prepared according to general procedure 1, starting from Intermediate N58 and 2-chloro-N-[4-(trifluoromethyl)phenyl]acetamide (CAS 2707-23-5). Purification by flash chromatography on silica gel (using a gradient of 0 to 50% EtOAc in iso-hexane as eluent) afforded the title compound as a white solid (58 mg, yield: 43%). LC-MS (Method A7) m/z [M+H]⁺: 475.2; rt: 2.36 min; purity: 98%. ¹H NMR (400 MHz, DMSO-d₆) δ 10.60 (s, 1H), 8.64 (dd, J=4.8, 1.7 Hz, 1H), 8.09 (ddd, J=7.9, 4.7, 1.8 Hz, 1H), 7.76 (d, J=8.5 Hz, 2H), 7.67 (d, J=8.5 Hz, 2H), 7.48 (dd, J=7.9, 4.7 Hz, 1H), 6.91 (s, 1H), 4.62 (s, 2H), 3.92 (s, 3H), 3.85 (q, J=6.6 Hz, 1H), 1.48 (d, J=6.6 Hz, 3H).

### Example #183: 2-(3-fluoro-5-methyl-9,12-dioxo-4,8,13-triazatricyclo[9.4.0.0^{2,7}]pentadeca-1(11),2(7),3,5,14-pentaen-8-yl)-N-[4-(trifluoromethyl)phenyl]acetamide

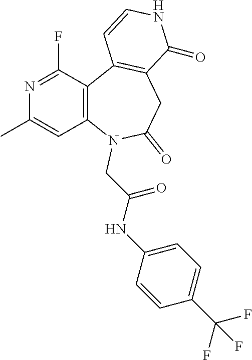 [SMILES: Cc1cc2c(c(F)n1)-c1cc[nH]c(=O)c1CC(=O)N2CC(=O)Nc1ccc(C(F)(F)F)cc1]

[2-(3-Fluoro-5-methyl-9,12-dioxo-4,8,13-triazatricyclo[9.4.0.0^{2,7}]pentadeca-1(11),2(7),3,5,14-pentaen-8-yl)acetyl]oxylithium (Intermediate N72, 31 mg, 0.070 mmol) was dissolved in dry DMF (3.0 mL) and 4-(trifluoromethyl)aniline (14 mg, 0.084 mmol), N,N-diisopropylethylamine (0.035 mL, 0.211 mmol) and HATU (40 mg, 0.105 mmol) were sequentially added and the reaction mixture was stirred at room temperature for 18 h. The reaction mixture was diluted with EtOAc (5 mL) and washed with water (2×5 mL) and brine (3×10 mL). The organic phase was dried over sodium sulfate, filtered and concentrated under vacuum. The residue was purified by flash chromatography on silica gel (using a gradient of 0 to 10% MeOH in DCM as eluent) to afford the title compound (1 mg, yield: 2%) as a white solid. LC-MS (Method A7) m/z [M+H]⁺: 461.2; rt: 1.9 min; purity: 99%. ¹H NMR (400 MHz, CD30D) δ 7.80 (d, J=8.5 Hz, 2H), 7.65 (d, J=8.5 Hz, 2H), 7.41 (d, J=6.9 Hz, 1H), 7.38 (s, 1H), 6.69 (dd, J=6.9, 5.3 Hz, 1H), 4.74 (d, J=16.7 Hz, 1H), 4.59 (d, J=16.7 Hz, 1H), 4.29 (d, J=13.1 Hz, 1H), 3.03 (d, J=13.1 Hz, 1H), 2.57 (s, 3H). 2 NH not observed.

### Example #184: enantiomer (10R) or (10S) of 2-(3-fluoro-5,10-dimethyl-4,9-dioxo-5,8,12-triazatricyclo[9.4.0.0^{2,7}]pentadeca-1(11),2,6,12,14-pentaen-8-yl)-N-[4-(trifluoromethyl)phenyl]acetamide

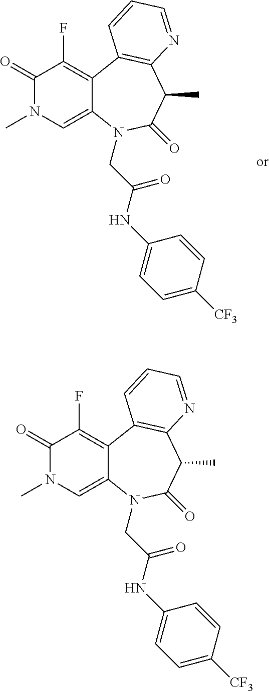 [SMILES: C[C@H]1C(=O)N(CC(=O)Nc2ccc(C(F)(F)F)cc2)c2cn(C)c(=O)c(F)c2-c2cccnc21 ; C[C@@H]1C(=O)N(CC(=O)Nc2ccc(C(F)(F)F)cc2)c2cn(C)c(=O)c(F)c2-c2cccnc21]

### Step 1: Preparation of 2-(3-fluoro-4-methoxy-10-methyl-9-oxo-5,8,12-triazatricyclo[9.4.0.0^{2,7}]-pentadeca-1(11),2,4,6,12,14-hexaen-8-yl)-N-[4-(trifluoromethyl)phenyl]acetamide #184_1

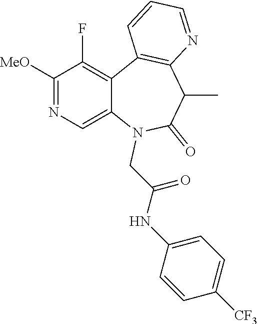 [SMILES: COc1ncc2c(c1F)-c1cccnc1C(C)C(=O)N2CC(=O)Nc1ccc(C(F)(F)F)cc1]

The title product was prepared according to general procedure 1, starting from 3-fluoro-4-methoxy-10-methyl-5,8,12-triazatricyclo[9.4.0.0^{2,7}]pentadeca-1(11),2,4,6,12,14-hexaen-9-one (Intermediate N59a, 285 mg, 1.02 mmol) and 2-chloro-N-[4-(trifluoromethyl)phenyl]acetamide (CAS 2707-23-5, 292 mg, 1.23 mmol, 1.20 eq.). Trituration in Et₂O afforded the title compound as a light pink solid (327 mg, yield: 65%). LC-MS (Method A1) m/z [M+H]⁺: 475.1; rt: 1.45 min; purity: 97%. ¹H NMR (400 MHz, DMSO-d₆) δ 10.52 (s, 1H), 8.71 (dd, J=4.8, 1.7 Hz, 1H), 8.32 (s, 1H), 8.18 (ddd, J=7.9, 4.0, 1.7 Hz, 1H), 7.72 (d, J=8.7 Hz, 2H), 7.66 (d, J=8.7 Hz, 2H), 7.54 (dd, J=7.9, 4.8 Hz, 1H), 4.65 (d, J=16.7 Hz, 1H), 4.54 (d, J=16.7 Hz, 1H), 4.02 (s, 3H), 3.85 (q, J=6.6 Hz, 1H), 1.47 (d, J=6.6 Hz, 3H). ¹⁹F NMR (376 MHz, DMSO-d₆) δ−60.32, −145.04 (d, J=4.0 Hz).

### Step 2: Preparation of 2-(3-fluoro-10-methyl-4,9-dioxo-5,8,12-triazatricyclo[9.4.0.0^{2,7}]pentadeca-1(11),2,6,12,14-pentaen-8-yl)-N-[4-(trifluoromethyl)phenyl]acetamide #184_2

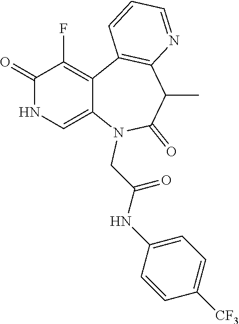 [SMILES: CC1C(=O)N(CC(=O)Nc2ccc(C(F)(F)F)cc2)c2c[nH]c(=O)c(F)c2-c2cccnc21]

To a suspension of 2-(3-fluoro-4-methoxy-10-methyl-9-oxo-5,8,12-triazatricyclo[9.4.0.0^{2,7}]-pentadeca-1 (11),2,4,6,12,14-hexaen-8-yl)-N-[4-(trifluoromethyl)phenyl]acetamide (Example #184_1, 303 mg, 0.62 mmol) in dry MeCN (3 mL) were added TMSCl (790 μL, 6.16 mmol) and KI (1.02 g, 6.16 mmol) and the reaction mixture was stirred at 80° C. for 16 h. After cooling down to room temperature, a saturated aqueous NaHCO₃ solution was added and the reaction mixture was extracted with EtOAc (2×). The combined organic layers were washed with brine, dried over Na₂SO₄, filtered and concentrated to dryness. The residue was purified by column chromatography on silica gel (using a gradient of 0% to 10% MeOH in EtOAc as eluent) to afford the title compound as a yellow solid (288 mg, yield: 92%). LC-MS (Method A1) m/z [M+H]⁺: 461; rt: 1.11 min; purity: 91%. ¹H NMR (400 MHz, DMSO-d₆) δ 12.54 (s, 1H), 10.50 (s, 1H), 8.66 (dd, J=4.8, 1.7 Hz, 1H), 8.11 (ddd, J=7.9, 4.0, 1.7 Hz, 1H), 7.74 (d, J=8.7 Hz, 2H), 7.70-7.64 (m, 3H), 7.51 (dd, J=7.9, 4.8 Hz, 1H), 4.46 (d, J=16.7 Hz, 1H), 4.41 (d, J=16.7 Hz, 1H), 4.09 (q, J=6.6 Hz, 1H), 1.44 (d, J=6.6 Hz, 3H).

### Step 3: Preparation of 2-(3-fluoro-5,10-dimethyl-4,9-dioxo-5,8,12-triazatricyclo[9.4.0.0^{2,7}]pentadeca-1(11),2,6,12,14-pentaen-8-yl)-N-[4-(trifluoromethyl)phenyl]acetamide #184_3

A suspension of 2-(3-fluoro-10-methyl-4,9-dioxo-5,8,12-triazatricyclo[9.4.0.0^{2,7}]pentadeca-1(11),2,6,12,14-pentaen-8-yl)-N-[4-(trifluoromethyl)phenyl]acetamide (#184_2, 270 mg, 0.53 mmol) and Cs₂CO₃ (530 mg, 1.61 mmol) in DMF (5 mL) was cooled to 0° C. before addition of iodomethane (34 μL, 0.53 mmol) and the reaction mixture was stirred at 0° C. for 2 h. A saturated aqueous NH₄Cl solution was added and the reaction mixture was extracted with EtOAc. The organic layer was washed with water (3×), brine, dried over Na₂SO₄, filtered and concentrated to dryness. The crude solid was purified by column chromatography on silica gel (using a gradient of 0% to 10% MeOH in EtOAc as eluent). After evaporation, the collected product was triturated in Et₂O, filtered and vacuum-dried to afford the racemic compound as an off-white solid (160 mg, yield: 61%). The racemic compound was purified by chiral SFC (column chiralpak AS from Daicel, using CO₂:EtOH 80:20) and then by basic preparative HPLC to yield the title compound as a white solid. LC-MS (Method B4) m/z [M+H]⁺: 475.2; rt: 3.45 min; purity: 96%. LC-MS (Method A4) m/z [M+H]⁺: 475.1; rt: 3.85 min; purity: 97%. ¹H NMR (400 MHz, DMSO-d₆) δ 10.48 (s, 1H), 8.66 (dd, J=4.8, 1.7 Hz, 1H), 8.10 (ddd, J=7.8, 3.9, 1.7 Hz, 1H), 7.94 (d, J=1.7 Hz, 1H), 7.73 (d, J=8.6 Hz, 2H), 7.66 (d, J=8.6 Hz, 2H), 7.51 (dd, J=7.8, 4.8 Hz, 1H), 4.49 (d, J=16.6 Hz, 1H), 4.39 (d, J=16.6 Hz, 1H), 4.11 (q, J=6.6 Hz, 1H), 3.59 (s, 3H), 1.44 (d, J=6.6 Hz, 3H). ¹⁹F NMR (376 MHz, DMSO-d₆) δ−60.31, −137.47 (d, J=3.9 Hz). Chiral purity: >99%; rt=1.67 min (first eluting enantiomer). For information, second eluting enantiomer rt=3.72 min. Both measured by HPLC, chiralpak AS from Daicel, EtOH 50%—heptane 50%—DEA 0.1%, Temp: 30° C.).

### Example #185: 2-(5-fluoro-3,10-dimethyl-4,9-dioxo-3,8,12-triazatricyclo[9.4.0.0^{2,7}]pentadeca-1(11),2(7),5,12,14-pentaen-8-yl)-N-[4-(trifluoromethyl)phenyl]acetamide

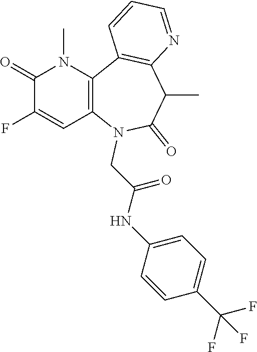 [SMILES: CC1C(=O)N(CC(=O)Nc2ccc(C(F)(F)F)cc2)c2cc(F)c(=O)n(C)c2-c2cccnc21]

### Step 1: Preparation of 2-(5-fluoro-4-methoxy-10-methyl-9-oxo-3,8,12-triazatricyclo[9.4.0.0^{2,7}]-pentadeca-1(11),2,4,6,12,14-hexaen-8-yl)-N-[4-(trifluoromethyl)phenyl]acetamide #185_1

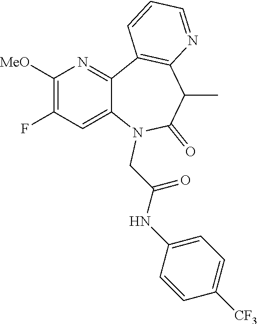 [SMILES: COc1nc2c(cc1F)N(CC(=O)Nc1ccc(C(F)(F)F)cc1)C(=O)C(C)c1ncccc1-2]

The title product was prepared following the general procedure 1, starting from 5-fluoro-4-methoxy-10-methyl-3,8,12-triazatricyclo[9.4.0.0^{2,7}]pentadeca-1(11),2,4,6,12,14-hexaen-9-one (Intermediate N59b, 270 mg, 0.89 mmol) and 2-chloro-N-[4-(trifluoromethyl)phenyl]acetamide (CAS 2707-23-5, 253 mg, 1.06 mmol). Trituration in Et₂O afforded the title compound as a white solid (237 mg, yield: 52%). LC-MS (Method A1) m/z [M+H]⁺: 475; rt: 1.47 min; purity: 93%. ¹H NMR (400 MHz, DMSO-d₆) δ 10.56 (s, 1H), 8.70 (dd, J=4.8, 1.7 Hz, 1H), 8.34 (dd, J=7.8, 1.7 Hz, 1H), 7.97 (d, J=11.3 Hz, 1H), 7.74 (d, J=8.6 Hz, 2H), 7.66 (d, J=8.6 Hz, 2H), 7.55 (dd, J=7.8, 4.8 Hz, 1H), 4.57 (d, J=16.7 Hz, 1H), 4.50 (d, J=16.7 Hz, 1H), 4.07 (s, 3H), 3.69 (q, J=6.6 Hz, 1H), 1.50 (d, J=6.6 Hz, 3H). ¹⁹F NMR (376 MHz, DMSO-d₆) δ−60.32, −138.64 (d, J=11.3 Hz).

### Step 2: Preparation of 2-(5-fluoro-10-methyl-4,9-dioxo-3,8,12-triazatricyclo[9.4.0.0^{2,7}]pentadeca-1(11),2(7),5,12,14-pentaen-8-yl)-N-[4-(trifluoromethyl)phenyl]acetamide #185_2

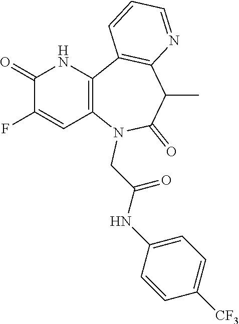 [SMILES: CC1C(=O)N(CC(=O)Nc2ccc(C(F)(F)F)cc2)c2cc(F)c(=O)[nH]c2-c2cccnc21]

To a suspension of 2-(5-fluoro-4-methoxy-10-methyl-9-oxo-3,8,12-triazatricyclo[9.4.0.0^{2,7}]-pentadeca-1(11),2,4,6,12,14-hexaen-8-yl)-N-[4-(trifluoromethyl)phenyl]acetamide (#185_1, 213 mg, 0.42 mmol) in dry MeCN (2 mL) were added TMSCl (530 μL, 4.16 mmol) and KI (685 mg, 4.16 mmol) and the resulting mixture was stirred at 80° C. for 16 h. Additional TMSCl (530 μL, 4.16 mmol) and KI (685 mg, 4.16 mmol) were added at RT and the reaction mixture was further stirred at 80° C. for 7 h. A saturated aqueous NaHCO₃ solution was added and the reaction mixture was extracted with EtOAc (2×). The combined organic layers were washed with brine, dried over Na₂SO₄, filtered and concentrated to dryness. The crude was purified by column chromatography on silica gel (using a gradient of 0% to 10% MeOH in EtOAc as eluent) to afford the title compound as a yellow solid (195 mg, quantitative yield). LC-MS (Method A1) m/z [M+H]⁺: 461.0; rt: 1.13 min; purity: 97%. ¹H NMR (400 MHz, DMSO-d₆) δ 12.53 (s, 1H), 10.52 (s, 1H), 8.70 (dd, J=4.8, 1.7 Hz, 1H), 8.13 (dd, J=7.9, 1.7 Hz, 1H), 7.78 (d, J=11.7 Hz, 1H), 7.74 (d, J=8.6 Hz, 2H), 7.66 (d, J=8.6 Hz, 2H), 7.52 (dd, J=7.9, 4.8 Hz, 1H), 4.49 (d, J=16.7 Hz, 1H), 4.38 (d, J=16.7 Hz, 1H), 3.80 (q, J=6.6 Hz, 1H), 1.49 (d, J=6.6 Hz, 3H).

### Step 3: Preparation of 2-(5-fluoro-3,10-dimethyl-4,9-dioxo-3,8,12-triazatricyclo[9.4.0.0^{2,7}]pentadeca-1(11),2(7),5,12,14-pentaen-8-yl)-N-[4-(trifluoromethyl)phenyl]acetamide #185_3

A suspension of 2-(5-fluoro-10-methyl-4,9-dioxo-3,8,12-triazatricyclo[9.4.0.0^{2,7}]pentadeca-1(11),2(7),5,12,14-pentaen-8-yl)-N-[4-(trifluoromethyl)phenyl]acetamide (#185_2, 193 mg, 0.41 mmol) and Cs₂CO₃ (402 mg, 1.22 mmol) in DMF (4 mL) was cooled to 0° C. before addition of iodomethane (26 μL, 0.41 mmol) and the reaction mixture was stirred at 0° C. for 2 h. A saturated aqueous NH₄Cl solution was added and the reaction mixture was extracted with EtOAc. The organic layer was washed with water (3×), brine, dried over Na₂SO₄, filtered and concentrated to dryness. The crude was purified by column chromatography on silica gel (using a gradient of 0% to 10% MeOH in EtOAc as eluent) and then by preparative HPLC (basic elution) to afford the title compound as a white solid (11 mg, yield: 6%). LC-MS (Method B4) m/z [M+H]⁺: 475.6; rt: 3.52 min; purity: 99%. LC-MS (Method A4) m/z [M+H]⁺: 475.8; rt: 3.89 min; purity: 99%. ¹H NMR (400 MHz, DMSO-d₆) δ 10.48 (s, 1H), 8.71 (dd, J=4.8, 1.7 Hz, 1H), 8.22 (dd, J=7.9, 1.7 Hz, 1H), 7.76 (d, J=11.2 Hz, 1H), 7.72 (d, J=8.7 Hz, 2H), 7.66 (d, J=8.7 Hz, 2H), 7.50 (dd, J=7.9, 4.8 Hz, 1H), 4.47 (d, J=16.7 Hz, 1H), 4.27 (d, J=16.7 Hz, 1H), 4.00 (q, J=6.6 Hz, 1H), 3.41 (s, 3H), 1.49 (d, J=6.6 Hz, 3H). ¹⁹F NMR (376 MHz, DMSO-d₆) δ−60.31, −130.44 (d, J=11.2 Hz).

### Example #186: enantiomer (7S) or (7R) of N-(4-chloro-3-fluoro-phenyl)-2-[7,12-dimethyl-8-oxo-5,6,9,13-tetrazatricyclo[8.4.0.0^{2,6}]tetradeca-1(10),2,4,11,13-pentaen-9-yl]acetamide

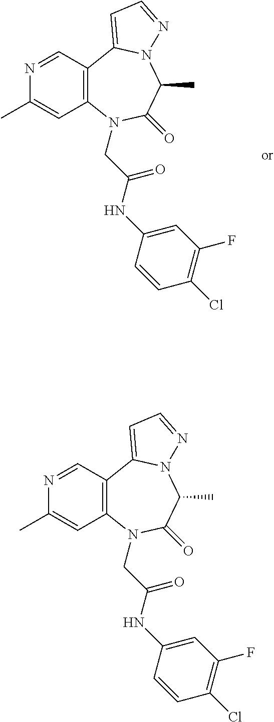 [SMILES: Cc1cc2c(cn1)-c1ccnn1[C@@H](C)C(=O)N2CC(=O)Nc1ccc(Cl)c(F)c1 ; Cc1cc2c(cn1)-c1ccnn1[C@H](C)C(=O)N2CC(=O)Nc1ccc(Cl)c(F)c1]

The title product was prepared according to the general procedure 1, starting from 7,12-dimethyl-5,6,9,13-tetrazatricyclo[8.4.0.0^{2,6}]tetradeca-1 (10),2,4,11,13-pentaen-8-one (Intermediate N77, 200 mg, 0.83 mmol) and 2-chloro-N-(4-chloro-3-fluoro-phenyl)acetamide (CAS 895641-02-8, 203 mg, 0.91 mmol). Purification by column chromatography on silica gel (using a gradient of 0% to 100% EtOAc in heptane as eluent) afforded the racemic compound as a white solid (292 mg, yield: 85%). The racemic compound was purified by chiral SFC (column chiralpak IA from Daicel, using CO₂:i-PrOH 80:20) to yield the title compound as a white solid. LC-MS (Method B2) m/z [M+H]⁺: 414.1; rt: 3.92 min; purity: 99%. LC-MS (Method A2) m/z [M+H]⁺: 414.1; rt: 3.85 min; purity>99%. ¹H NMR (400 MHz, DMSO-d₆) δ 10.63 (s, 1H), 8.72 (s, 1H), 7.76 (dd, J=11.8, 2.2 Hz, 1H), 7.63 (d, J=1.9 Hz, 1H), 7.54 (t, J=8.7 Hz, 1H), 7.38 (s, 1H), 7.33 (dd, J=8.7, 2.2 Hz, 1H), 6.83 (d, J=1.9 Hz, 1H), 4.87 (br s, 1H), 4.66 (d, J=16.6 Hz, 1H), 4.57 (d, J=16.6 Hz, 1H), 2.56 (s, 3H), 1.70 (br s, 3H). Chiral purity: 99%; rt=2.0 min (second eluting enantiomer). For information, first eluting enantiomer rt=1.69 min. Both measured by HPLC, Chiralpak IA from Daicel, EtOH 50%—heptane 50%—DEA 0.1%).

### Example #187: enantiomer (10R) or (10S) of 2-[3-fluoro-10-methyl-9-oxo-4,8,12-triazatricyclo[9.4.0.0^{2,7}]pentadeca-1(11),2(7),3,5,12,14-hexaen-8-yl]-N-[4-(trifluoromethyl)phenyl]acetamide

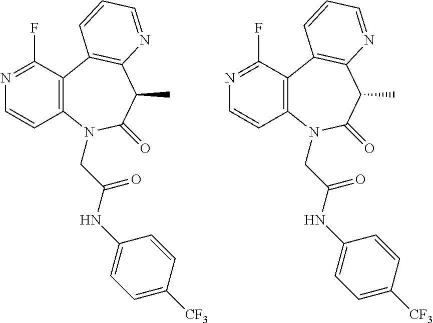 [SMILES: C[C@H]1C(=O)N(CC(=O)Nc2ccc(C(F)(F)F)cc2)c2ccnc(F)c2-c2cccnc21 ; C[C@@H]1C(=O)N(CC(=O)Nc2ccc(C(F)(F)F)cc2)c2ccnc(F)c2-c2cccnc21]

The title product was prepared according to the general procedure 1, starting from 3-fluoro-10-methyl-4,8,12-triazatricyclo[9.4.0.0^{2,7}]pentadeca-1(11),2(7),3,5,12,14-hexaen-9-one (Intermediate N78, 45 mg, 0.18 mmol) and 2-chloro-N-[4-(trifluoromethyl)phenyl]acetamide (CAS 2707-23-5, 42 mg, 0.18 mmol). Upon treatment with water, the formed precipitate was filtered, triturated in Et₂O, filtered and vacuum-dried to afford the racemic compound as a white solid (60 mg, yield: 76%). The racemic compound was purified by chiral SFC (column chiralpak IA from Daicel, using CO₂:EtOH 80:20) to yield the title compound as a white solid. LC-MS (Method B2) m/z [M+H]⁺: 445.1; rt: 4.26 min; purity: 99%. LC-MS (Method A2) m/z [M+H]⁺: 445.1; rt: 4.49 min; purity: 98%. ¹H NMR (400 MHz, DMSO-d₆) δ 10.64 (s, 1H), 8.70 (dd, J=4.8, 1.7 Hz, 1H), 8.30 (d, J=5.6 Hz, 1H), 8.17 (ddd, J=7.9, 4.7, 1.7 Hz, 1H), 7.76 (d, J=8.6 Hz, 2H), 7.68 (d, J=8.6 Hz, 2H), 7.52 (dd, J=7.9, 4.8 Hz, 1H), 7.50 (d, J=5.6 Hz, 1H), 4.66 (d, J=16.9 Hz, 1H), 4.61 (d, J=16.9 Hz, 1H), 3.81 (q, J=6.6 Hz, 1H), 1.51 (d, J=6.6 Hz, 3H). Chiral purity: >99%; rt=2.43 min (second eluting enantiomer). For information, first eluting enantiomer rt=1.83 min, both measured by HPLC, Chiralpak IA from Daicel, EtOH 50%—heptane 50%—DEA 0.1%).

### Example #188: enantiomer (10R) or (10S) of 2-[3-chloro-10-fluoro-5-methyl-9-oxo-4,8,12-triazatricyclo[9.4.0.0^{2,7}]pentadeca-1(11),2(7),3,5,12,14-hexaen-8-yl]-N-[4-(trifluoromethyl)phenyl]acetamide

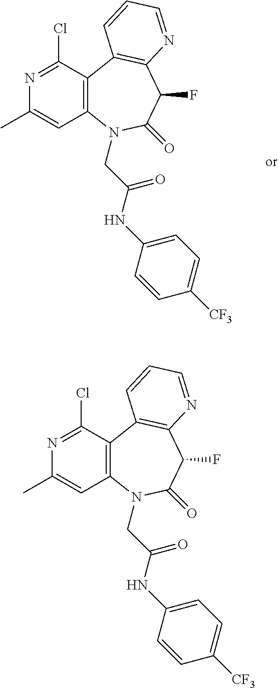 [SMILES: Cc1cc2c(c(Cl)n1)-c1cccnc1[C@@H](F)C(=O)N2CC(=O)Nc1ccc(C(F)(F)F)cc1 ; Cc1cc2c(c(Cl)n1)-c1cccnc1[C@H](F)C(=O)N2CC(=O)Nc1ccc(C(F)(F)F)cc1]

The title product was prepared according to the general procedure 1, starting from intermediate N60 (75 mg, 0.22 mmol) and 2-chloro-N-[4-(trifluoromethyl)phenyl]acetamide (CAS 2707-23-5). After complete conversion, water was added to the reaction mixture and the resulting precipitate was collected by filtration and dried under vacuum. After purification by reverse phase HPLC (basic elution), the racemate was separated by Chiral HPLC (column CHIRALPAK IG from Daicel, eluant EtOH-Heptane 1:1+0.1% DEA) to yield the pure enantiomer as a white solid (2 mg, yield: 3%). LC-MS (Method B3) m/z [M+H]⁺: 479.4; rt: 3.81 min; purity: 93%. LC-MS (Method A3) m/z [M+H]⁺: 479.4; rt: 4.29 min; purity: 92%. ¹H NMR (400 MHz, DMSO-d₆) δ 10.63 (s, 1H), 8.71 (dd, J=4.8, 1.6 Hz, 1H), 8.29 (dt, J=7.9, 1.6 Hz, 1H), 7.74 (d, J=8.7 Hz, 2H), 7.67 (d, J=8.7 Hz, 2H), 7.59 (dd, J=7.9, 4.8 Hz, 1H), 7.48 (s, 1H), 6.45-6.23 (d, J=110 Hz, 1H), 4.82-4.47 (m, 2H), 2.54 (s, 3H). Chiral purity: 97%; rt=3.88 min (second eluting enantiomer). For information, first eluting enantiomer rt=3.09 min. Both measured by HPLC, Chiralpak IG-u from Daicel, EtOH 30%—heptane 70%—DEA 0.1%)

The following Examples #189-211 have been prepared by chiral purification of corresponding racemates identified in the table hereafter.

| | Structure | | |
|---|---|---|---|
| Ex. | Example | Example Name | Protocolumn and characterization |
| #189 | 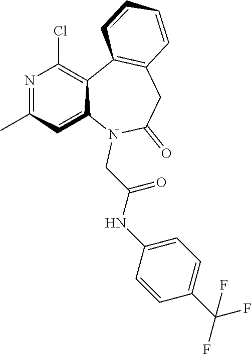 | Atropisomer aR or aS of 2-(1-chloro-3-methyl- 6-oxo-7H-pyrido[4,3- d][3]benzazepin-5-yl)- N-[4- (trifluoromethyl)phenyl] acetamide | Example #49 was purified by chiral HPLC (column Whelk-O1 (R,R) from Regis Technology) using hep- tane:isopropanol (1:1) and DEA (0.1%) to yield compound #189. LC-MS (Method B2) m/z [M + H]⁺; 460.0; rt: 4.66 min; purity >99%. LC-MS (Method A2) m/z [M + H]⁺: 460.0; rt: 4.73 min; purity >99%. ¹H NMR (400 MHz, DMSO-d₆) δ 10.59 (s, 1H), 7.81-7.78 (m, 3H), 7.69 (d, J = 8.5 Hz, 2H), 7.49-7.44 (m, 3H), 7.39 (s, 1H), 4.51 (s, 2H), 3.56 (s, 2H). CH₃ under solvent peak. Chiral purity: 95%; rt = 2.68 min (first eluting atropisomer). For information, second eluting atropisomer rt = 2.94 min. Both measured by HPLC, Whelk O-1 (R,R) from Regis Technology (3μ); i-PrOH 50% - heptane 50% - DEA 0.1%) |
| | or | | |
| | 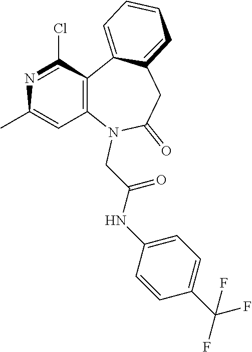 | | |
| #190 | 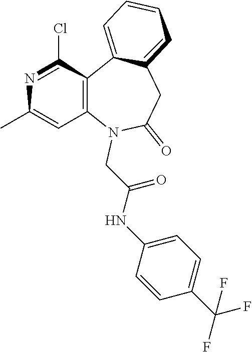 | Atropisomer aS or aR of 2-(1-chloro-3-methyl- 6-oxo-7H-pyrido[4,3- d][3]benzazepin-5-yl)- N-[4- (trifluoromethyl)phenyl] acetamide | Example #49 was purified by chiral HPLC (column Whelk-O1 (R,R) from Regis Technology) using heptane:iso- propanol (1:1) and DEA (0.1%) to yield compound #190. LC-MS (Method B2) m/z [M + H]⁺: 460.0; rt: 4.66 min; purity >99%. LC-MS (Method A2) m/z [M + H]⁺: 460.0; rt: 4.73 min; purity >99%. ¹H NMR (400 MHz, DMSO-d₆) δ 10.59 (s, 1H), 7.81-7.78 (m, 3H), 7.69 (d, J = 8.5 Hz, 2H), 7.49-7.44 (m, 3H), 7.39 (s, 1H), 4.51 (s, 2H), 3.56 (s, 2H). Chiral purity: 96%; rt = 2.94 min (second eluting atropisomer). For information, first eluting atropisomer rt = 2.68 min. Both measured by HPLC, Whelk O-1 (R,R) from Regis Technology (3μ); i-PrOH 50% - heptane 50% - DEA 0.1%) |
| | or | | |
| | 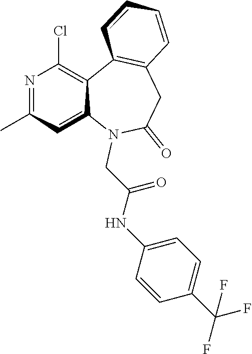 | | |
| #191 | 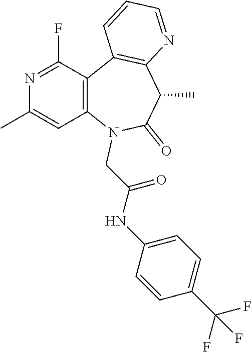 [SMILES: Cc1cc2c(c(F)n1)-c1cccnc1[C@H](C)C(=O)N2CC(=O)Nc1ccc(C(F)(F)F)cc1] | 2-[(10S)-3-fluoro-5,10- dimethyl-9-oxo-4,8,12- triazatricyclo[9.4.0.0^{2,7}] pentadeca- 1(11),2(7),3,5,12,14- hexaen-8-yl]-N-[4- (trifluoromethyl)phenyl] acetamide | Example #84 was purified by chiral SFC (column chiralpak AD from Daicel, using CO₂: isopropanol 80:20) to yield compound #191. LC-MS (Method B2′) m/z [M + H]⁺: 459.0; rt: 4.56 min; purity: 98%. LC-MS (Method Method A2′) m/z [M + H]⁺: 459.0; rt: 4.60 min; purity: 98%. ¹H NMR (400 MHz, DMSO-d₆) δ 10.60 (s, 1H), 8.67 (dd, J = 4.8, 1.8 Hz, 1H), 8.13 (ddd, J = 7.9, 4.8, 1.8 Hz, 1H), 7.76 (d, J = 8.8 Hz, 2H), 7.68 (d, J = 8.8 Hz, 2H), 7.50 (dd, J = 7.9, 4.8 Hz, 1H), 7.38 (s, 1H), 4.60 (s, 2H), 3.79 (q, J = 6.6 Hz, 1H), 2.50 (s, 3H), 1.49 (d, J = 6.6 Hz, 3H). Chiral purity: 97%; rt = 1.63 min (first eluting enantiomer). For information, second eluting enantiomer rt = 2.09 min. Both measured by HPLC, Chiralpak IA from Daicel, EtOH 50% - heptane 50% - DEA 0.1%) |
| #192 | 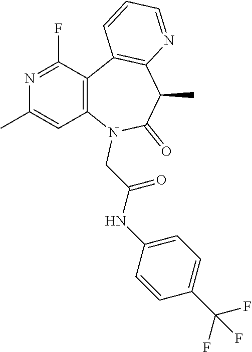 [SMILES: Cc1cc2c(c(F)n1)-c1cccnc1[C@@H](C)C(=O)N2CC(=O)Nc1ccc(C(F)(F)F)cc1] | 2-[(10R)-3-fluoro-5,10- dimethyl-9-oxo-4,8,12- triazatricyclo[9.4.0.0^{2,7}] pentadeca- 1(11),2(7),3,5,12,14- hexaen-8-yl]-N-[4- (trifluoromethyl)phenyl] acetamide | Example #84 was purified by chiral SFC (column chiralpak AD from Daicel, using CO₂:isopropanol 80:20) to yield compound #192. LC-MS (Method B2′) m/z [M + H]⁺: 459.0; rt: 4.56 min; purity: 98%. LC-MS (Method A2′) m/z [M + H]⁺: 459.0; rt: 4.60 min; purity: 98%. ¹H NMR (400 MHz, DMSO-d₆) δ 10.60 (s, 1H), 8.67 (dd, J = 4.8, 1.8 Hz, 1H), 8.13 (ddd, J = 7.9, 4.8, 1.8 Hz, 1H), 7.76 (d, J = 8.8 Hz, 2H), 7.68 (d, J = 8.8 Hz, 2H), 7.50 (dd, J = 7.9, 4.8 Hz, 1H), 7.38 (s, 1H), 4.60 (s, 2H), 3.79 (q, J = 6.6 Hz, 1H), 2.50 (s, 3H), 1.49 (d, J = 6.6 Hz, 3H). Chiral purity: 98%; rt = 2.09 min (second eluting enantiomer). For information, first eluting enantiomer rt = 1.63 min. Both measured by HPLC, Chiralpak IA from Daicel, EtOH 50% - heptane 50% - DEA 0.1%) |
| #193 | 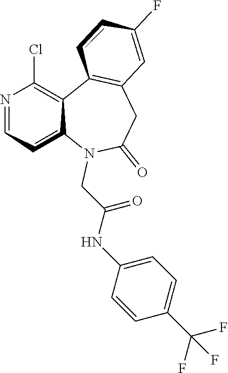 | Atropisomer aR or aS of 2-(1-chloro-9-fluoro- 6-oxo-7H-pyrido[4,3- d][3]benzazepin-5-yl)- N-[4- (trifluoromethyl)phenyl] acetamide | Example #51 was purified by chiral HPLC (column chiralpak IG from Daicel) using heptane:ethanol (1:1) and DEA (0.1%) to yield compound #193. LC-MS (Method B2) m/z [M − H]⁻: 462.1; rt: 4.61 min; purity: 97%. LC- MS (Method A2) m/z [M + H]⁺: 463.9; rt: 4.75 min; purity: 94%. ¹H NMR (400 MHz, DMSO-d₆) δ 10.60 (s, 1H), 8.43 (d, J = 5.5 Hz, 1H), 7.88 (dd, J = 8.8, 5.5 Hz, 1H), 7.77 (d, J = 8.8 Hz, 2H), 7.68 (d, J = 8.9 Hz, 2H), 7.52 (d, J = 5.6 Hz, 1H), 7.41 (dd, J = 9.3, 2.8 Hz, 1H), 7.30 (td, J = 8.7, 2.8 Hz, 1H), 4.56 (s, 2H), 3.61 (s, 2H). Chiral purity: >99%; rt = 2.9 min (second eluting atropisomer). For information, first eluting atropisomer rt = 1.83 min. Both measured by HPLC, Chiralpak IG from Daicel, EtOH 50% - heptane 50% - DEA 0.1%) |
| | or | | |
| | 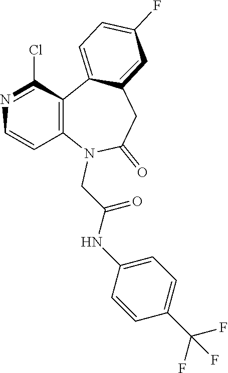 | | |
| #194 | 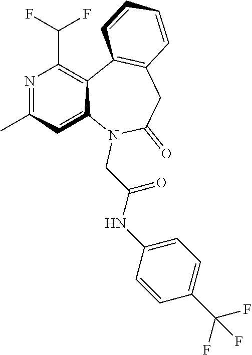 | Atropisomer aR or aS of 2-[1-(difluoromethyl)- 3-methyl-6-oxo-7H- pyrido[4,3- d][3]benzazepin-5-yl]- N-[4- (trifluoromethyl)phenyl] acetamide | Example #56 was purified by chiral HPLC (column chiralpak IA from Daicel) using heptane:ethanol (1:1) and DEA (0.1%) to yield compound #194. LC-MS (Method B3) u/z [M + H]⁺: 476.14; rt: 5.02 min; purity: 99%. LC-MS (Method A3) m/z [M + H]⁺: 476.4; rt: 4.61 min; purity: 98%. ¹H NMR (400 MHz, DMSO-d₆) δ 10.59 (s, 1H), 7.79 (d, J = 8.9 Hz, 2H), 7.69 (d, J = 8.9 Hz, 2H), 7.58-7.47 (m, 4H), 7.44 (d, J = 7.2 Hz, 1h), 6.75 (t, J = 53.6 Hz, 1H), 4.55 (s, 2H), 3.60 (d, J = 12.8 Hz, 1H), 3.56 (d, J = 12.8 Hz, 1H), 2.60 (s, 3H). Chiral purity measured just after chiral separation: 92%; rt = 2.73 min (second eluting atropisomer). For information, first eluting atropisomer rt = 1.51 min. Both measured by HPLC, Chiralpak IA from Daicel, EtOH 50% - heptane 50% - DEA 0.1%). Chiral purity of #194 |
| | or | | dropped over time upon standing at |
| | 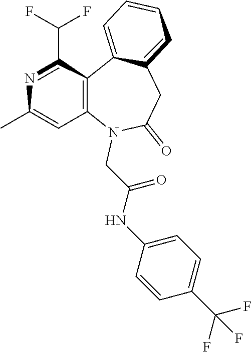 | | RT, indicating interconversion of atropisomers. |
| #195 | 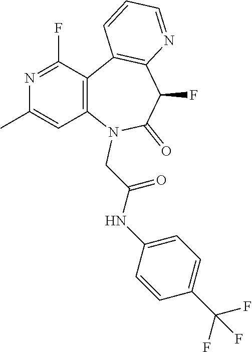 [SMILES: Cc1cc2c(c(F)n1)-c1cccnc1[C@@H](F)C(=O)N2CC(=O)Nc1ccc(C(F)(F)F)cc1] | Enantiomer (10R) or (10S) of 2-(3,10- difluoro-5-methyl-9- oxo-4,8,12- triazatricyclo[9.4.0.0^{2,7}] pentadeca- 1(11),2(7),3,5,12,14- hexaen-8-yl)-N-[4- (trifluoromethyl)phenyl] acetamide | Example #101 was purified by chiral SFC (column Whelk-O1 (R,R) from Regis Technology, using CO₂:eth- anol 90:10) to yield compound #195. LC-MS (Method B3) m/z [M + H]⁺: 463.0; rt: 4.17 min; purity: 96%. LC-MS (Method A3) m/z [M + H]⁺: 463.0; rt: 4.73 min; purity: 95%. ¹H NMR (400 MHz, DMSO-d₆) δ 10.64 (s, 1H), 8.73 (dd, J = 4.8, 1.7 Hz, 1H), 8.20 (dd, J = 7.9, 1.7 Hz, 1H), 7.76 (d, J = 8.8 Hz, 2H), 7.68 (d, J = 8.8 Hz, 2H), 7.61 (dd, J = 7.9, 4.8 Hz, 1H), 7.40 (s, 1H), 6.29 (d, J = 45.3 Hz, 1H), 4.67 (d, J = 16.9 Hz, 1H), 4.64 (d, J = 16.9 Hz, 1H) 2.49 (s, 3H). Chiral purity: 96%; rt = 6.06 min (first eluting enantiomer). For information, second eluting enantiomer rt = 7.84 min. Both measured by HPLC, Whelk O-1 (R,R) from Regis Technology (3μ); i-PrOH 50% - heptane 50% - DEA 0.1%) |
| | or | | |
| | 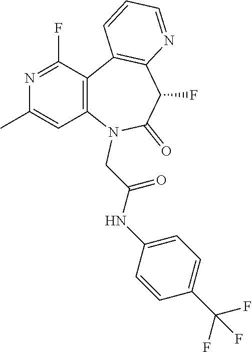 [SMILES: Cc1cc2c(c(F)n1)-c1cccnc1[C@H](F)C(=O)N2CC(=O)Nc1ccc(C(F)(F)F)cc1] | | |
| #196 | 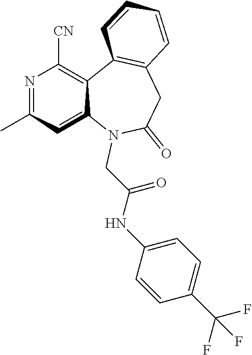 | Atropisomer aR or aS of 2-(1-cyano-3-methyl- 6-oxo-7H-pyrido[4,3- d][3]benzazepin-5-yl)- N-[4- (trifluoromethyl)phenyl] acetamide | Example #61 was purified by chiral HPLC (column chiralpak IA from Daicel, using heptane:ethanol 1:1 and 0.1% DEA) to yield compound #196. LC-MS (Method B3) m/z [M + H]⁺: 451.14; rt: 4.82 min; purity >99%. LC-MS ((Method A3) m/z [M + H]⁺: 451.04; rt: 508 min; purity: 99%. ¹H NMR (400 MHz, DMSO-d₆) δ 10.68 (s, 1H), 7.90 (d, J = 8.3 Hz, 1H), 7.79 (d, J = 8.6 Hz, 2H), 7.69 (d, J = 8.6 Hz, 2H), 7.66 (s, 1H), 7.60-7.52 (m, 3H), 4.60 (d, J = 16.8 Hz, 1H), 4.54 (d, J = 16.8 Hz, 1H), 3.67 (d, J = 12.8 Hz, 1H), 3.62 (d, J = 12.8 Hz, 1H), 2.59 (s, 3H). Chiral purity: 87%; rt = 3 min (second eluting atropisomer). For information, first eluting atropisomer rt = 1.71 min. Both measured by HPLC, Chiralpak IA from Daicel, EtOH 50% - heptane 50% - DEA 0.1%) |
| | or | | |
| | 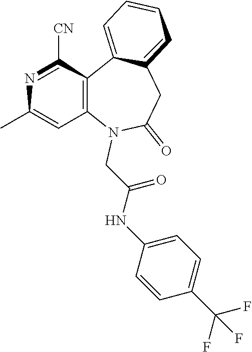 | | |
| #197 | 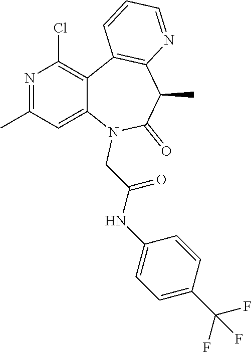 [SMILES: Cc1cc2c(c(Cl)n1)-c1cccnc1[C@@H](C)C(=O)N2CC(=O)Nc1ccc(C(F)(F)F)cc1] | Enantiomer (10R) or (10S) of 2-[3-chloro- 5,10-dimethyl-9-oxo- 4,8,12- triazatricyclo[9.4.0.0^{2,7}] pentadeca- 1(11),2,4,6,12,14- hexaen-8-yl]-N-[4- (trifluoromethyl)phenyl] acetamide | Example #114 was purified by chiral SFC (column chiralpak AD from Daicel, using CO₂:isopropanol 80:20) to yield compound #197. LC-MS (Method B2) m/z [M + H]⁺: 475.0; rt: 4.52 min; purity: 97%. LC-MS (Method A2) m/z [M + H]⁺: 475.0; rt: 4.50 min; purity: 96%. ¹H NMR (400 MHz, DMSO-d₆) δ 10.59 (s, 1H), 8.66 (dd, J = 4.8, 1.7 Hz, 1H), 8.24 (dd, J = 7.9, 1.7 Hz, 1H), 7.85-7.60 (m, 4H), 7.55- 7.39 (m, 2H), 4.72-4.38 (m, 2H), 3.82 (q, J = 6.6 Hz, 1H), 2.54 (s, 3H), 1.49 (d, J = 6.6 Hz, 3H). Chiral purity: 96%; rt = 2.0 min (second eluting enantiomer). For information, first eluting enantiomer rt = 1.52 min. Both measured by HPLC, Chiralpak AD from Daicel, i-PrOH 50% - heptane 50% - DEA 0.1%) |
| | or | | |
| | 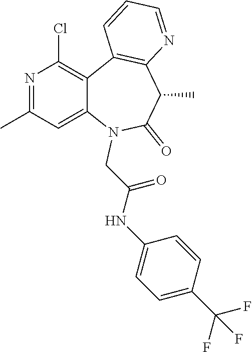 [SMILES: Cc1cc2c(c(Cl)n1)-c1cccnc1[C@H](C)C(=O)N2CC(=O)Nc1ccc(C(F)(F)F)cc1] | | |
| #198 | 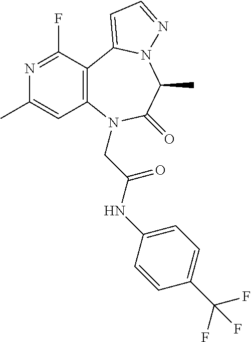 [SMILES: Cc1cc2c(c(F)n1)-c1ccnn1[C@@H](C)C(=O)N2CC(=O)Nc1ccc(C(F)(F)F)cc1] | Enantiomer (7R) or (7S) of 2-[14-fluoro- 7,12-dimethyl-8-oxo- 5,6,9,13- tetrazatricyclo[8.4.0.0^{2,6}] tetradeca- 1(10),2,4,11,13- pentaen-9-yl]-N-[4- (trifluoromethyl)phenyl] acetamide | Example #111 was purified by chiral HPLC (column chiralpak IG from Daicel) using heptane:ethanol (8:2) and DEA (0.1%) to yield compound #198. LC-MS (Method B4) m/z [M + H]⁺: 448.5; rt: 4.03 min; purity >99%. LC-MS (Method A4) m/z [M + H]⁺: 448.4; rt: 4.51 min; purity >99%. Chiral purity: 99%; rt = 1.91 min (second eluting enantiomer). For information, first eluting enantiomer rt = 1.61 min. Both measured by HPLC, Chiralpak IG-u from Daicel, EtOH 50% - heptane 50% - DEA 0.1%) |
| | or | | |
| | 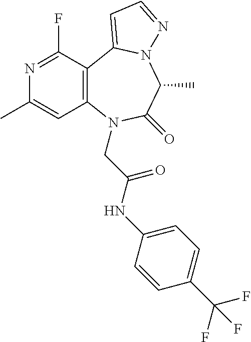 [SMILES: Cc1cc2c(c(F)n1)-c1ccnn1[C@H](C)C(=O)N2CC(=O)Nc1ccc(C(F)(F)F)cc1] | | |
| #199 | 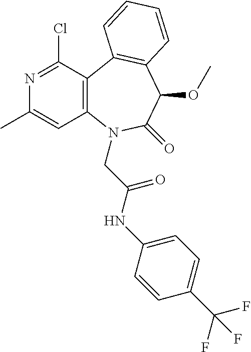 [SMILES: CO[C@H]1C(=O)N(CC(=O)Nc2ccc(C(F)(F)F)cc2)c2cc(C)nc(Cl)c2-c2ccccc21] | Entnaiomer (7R) or (7S) of 2-[1-chloro-7- methoxy-3-methyl-6- oxo-7H-pyrido[4,3- d][3]benzazepin-5-yl]- N-[4- (trifluoromethyl)phenyl] acetamide | Example #107 was purified by chiral SFC (column chirallpak IA from Daicel, using CO₂:ethanol 80:20) to yield compound #199. LC-MS (Method B2) m/z [M + H]⁺: 490.0; rt: 4.66 min; purity: 98%. LC-MS (Method A2) m/z [M + H]⁺: 490.3; rt: 4.80 min; purity: 98%. ¹H NMR (400 MHz, DMSO-d₆) δ 10.61 (s, 1H), 7.79 (d, J = 8.7 Hz, 3H), 7.70 (d, J = 8.8 Hz, 2H), 7.57-7.50 (m, 2H), 7.46 (td, J = 7.2, 2.1 Hz, 1H), 7.39 (s, 1H), 4.83 (s, 1H), 4.56 (d, J = 1.6 Hz, 2H), 3.43 (s, 3H), 2.53 (s, 3H). Chiral purity: >99%; rt = 2.2 min (second eluting enantiomer). For information, first eluting enantiomer rt = 1.37 min. Both measured by HPLC, Chiralpak IG-u from Daicel, EtOH 50% - heptane 50% - DEA 0.1%) |
| | or | | |
| | 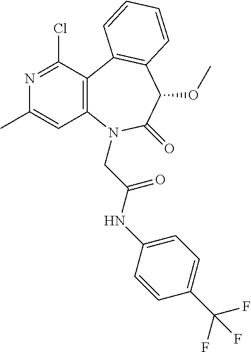 [SMILES: CO[C@@H]1C(=O)N(CC(=O)Nc2ccc(C(F)(F)F)cc2)c2cc(C)nc(Cl)c2-c2ccccc21] | | |
| #200 | 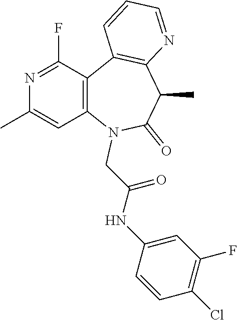 [SMILES: Cc1cc2c(c(F)n1)-c1cccnc1[C@@H](C)C(=O)N2CC(=O)Nc1ccc(Cl)c(F)c1] | Enantiomer (10R) or (10S) of N-(4-chloro-3- fluoro-phenyl)-2-[3- fluoro-5,10-dimethyl-9- oxo-4,8,12- triazatricyclo[9.4.0.0^{2,7}] pentadeca- 1(11),2(7),3,5,12,14- hexaen-8-yl]acetamide | Example #90 was purified by chiral HPLC (column chiralpak IG from Daicel, using methanol and 0.1% DEA) to yield compound #200. LC-MS (Method B2) m/z [M + H]⁺: 443.0; rt: 4.44 min; purity: 97%. LC-MS (Method A2) m/z [M + H]⁺: 443.0; rt: 4.47 min; purity: 95%. ¹H NMR (400 MHz, DMSO-d₆) δ 10.58 (s, 1H), 8.67 (dd, J = 4.8, 1.8 Hz, 1H), 8.12 (ddd, J = 7.9, 4.6, 1.6 Hz, 1H), 7.73 (dd, J = 11.9, 2.4 Hz, 1H), 7.57-7.46 (m, 2H), 7.36 (s, 1H), 7.31 (dd, J = 8.2, 1.9 Hz, 1H), 4.56 (s, 2H), 3.78 (q, J = 6.7 Hz, 1H), 1.49 (d, J = 6.7 Hz, 3H). Chiral purity: 98%; rt = 3.36 min (second eluting enantiomer). For information, first eluting enantiomer: rt = 1.90 min. Both measured by HPLC, Chiralpak IG from Daicel, MeOH 100% - DEA 0.1%) |
| | or | | |
| | 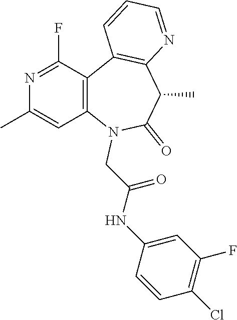 [SMILES: Cc1cc2c(c(F)n1)-c1cccnc1[C@H](C)C(=O)N2CC(=O)Nc1ccc(Cl)c(F)c1] | | |
| #201 | 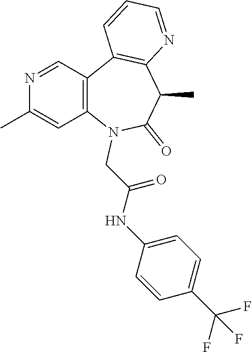 [SMILES: Cc1cc2c(cn1)-c1cccnc1[C@@H](C)C(=O)N2CC(=O)Nc1ccc(C(F)(F)F)cc1] | Enantiomer (10R) or (10S) of 2-[5,10- dimethyl-9-oxo-4,8,12- triazatricyclo[9.4.0.0^{2,7}] pentadeca- 1(11),2(7),3,5,12,14- hexaen-8-yl]-N-[4- (trifluoromethyl)phenyl] acetamide | Example #91 was purified by chiral SFC (column chiralpak IA from Daicel, using CO₂:ethanol 80:20) to yield compound #201. LC-MS (Method B2) m/z [M + H]⁺: 441.0; rt: 4.06 min; purity >99%. LC-MS (Method A2) m/z [M + H]⁺: 441.0; rt: 3.42 min; purity: 97%. ¹H NMR (400 MHz, DMSO-d₆) δ 10.53 (s, 1H), 8.66 (s, 1H), 8.61 (dd, J = 4.8, 1.8 Hz, 1H), 8.09 (dd, J = 7.8, 1.8 Hz, 1H), 7.71 (d, J = 8.9 Hz, 2H), 7.61 (d, J = 8.9 Hz, 2H), 7.46 (dd, J = 7.8, 4.8 Hz, 1H), 7.31 (s, 1H), 4.57 (d, J = 16.9 Hz, 1H), 4.50 (d, J = 16.9 Hz, 1H), 3.58 (q, J = 6.7 Hz, 1H), 2.49 (s, 3H), 1.43 (d, J = 6.7 Hz, 3H). Chiral purity: >99%; rt = 2.78 min (second eluting enantiomer). For information, first eluting enantiomer rt = 1.81 min. Both measured by HPLC, Chiralpak IG from Daicel, EtOH 50% - heptane 50% - DEA 0.1%) |
| | or | | |
| | 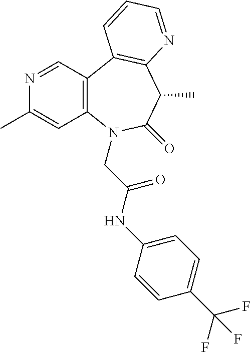 [SMILES: Cc1cc2c(cn1)-c1cccnc1[C@H](C)C(=O)N2CC(=O)Nc1ccc(C(F)(F)F)cc1] | | |
| #202 | 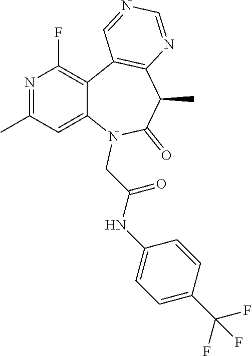 [SMILES: Cc1cc2c(c(F)n1)-c1cncnc1[C@@H](C)C(=O)N2CC(=O)Nc1ccc(C(F)(F)F)cc1] | Enantiomer (8R) or (8S) of 2-[15-fluoro- 8,13-dimethyl-9-oxo- 4,6,10,14- tetrazatricyclo[9.4.0.0^{2,7}] pentadeca- 1(15),2(7),3,5,11,13- hexaen-10-yl]-N-[4- (trifluoromethyl)phenyl] acetamide | Example #112 was purified by chiral HPLC (column chiralpak AD from Daicel, using heptane:ethanol 1:1 and 0.1% DEA) to yield compound #202. LC-MS (Method B4) m/z [M + H]⁺: 460.1; rt: 4.24 min; purity >99%. LC-MS (Method A4) m/z [M + H]⁺: 460.1; rt: 4.43 min; purity: 98%. ¹H NMR (400 MHz, DMSO-d₆) δ 10.65 (s, 1H), 9.27 (s, 1H), 9.15 (d, J = 4.6 Hz, 1H), 7.77 (d, J = 8.5 Hz, 2H), 7.68 (d, J = 8.7 Hz, 2H), 7.43 (s, 1H), 4.63 (s, 2H), 3.93 (d, J = 6.5 Hz, 1H), 2.53 (s, 3H), 1.49 (d, J = 6.6 Hz, 3H). Chiral purity: 92%; rt = 3.5 min (second eluting enantiomer). For information, first eluting enantiomer rt = 1.73 min. Both measured by HPLC, Chiralpak AD form Daicel, EtOH 50% - heptane 50% - DEA 0.1%) |
| | or | | |
| | 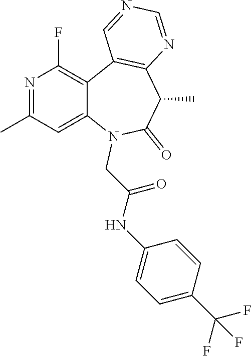 [SMILES: Cc1cc2c(c(F)n1)-c1cncnc1[C@H](C)C(=O)N2CC(=O)Nc1ccc(C(F)(F)F)cc1] | | |
| #203 | 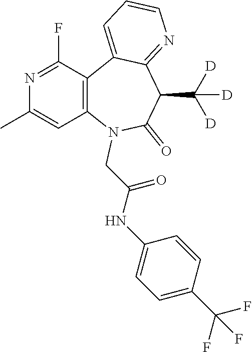 [SMILES: [2H]C([2H])([2H])[C@H]1C(=O)N(CC(=O)Nc2ccc(C(F)(F)F)cc2)c2cc(C)nc(F)c2-c2cccnc21] | Enantiomer (10R) or (10S) of 2-[3-fluoro-5- methyl-9-oxo-10- (trideuteriomethyl)- 4,8,12- triazatricyclo[9.4.0.0^{2,7}] pentadeca- 1(15),2(7),3,5,11,13- hexaen-8-yl]-N-[4- (trifluoromethyl)phenyl] acetamide | Example #92 was purified by chiral HPLC (column Whelk-O1 (R,R) from Regis Technology, using heptane:iso- propanol 1:1 and 0.1% DEA) to yield compound #203. LC-MS (Method B2) m/z [M + H]⁺: 462.4; rt: 4.49 min; purity: 97%. LC-MS (Method A2) m/z [M + H]⁺: 462.4; rt: 4.53 min; purity: 97%. ¹H NMR (400 MHz, DMSO-d₆) δ 10.60 (s, 1H), 8.67 (dd, J = 4.8, 1.8 Hz, 1H), 8.13 (ddd, J = 7.9, 4.8, 1.8 Hz, 1H), 7.77 (d, J = 8.8 Hz, 2H), 7.68 (d, J = 8.8 Hz, 2H), 7.50 (dd, J = 7.9, 4.8 Hz, 1H), 7.38 (s, 1H), 4.60 (s, 2H), 3.77 (s, 1H), 2.52 (s, 3H). Chiral purity: >99%; rt = 3.03 min (first eluting enantiomer). For information, second eluting enantiomer rt = 4.00 min. Both measured by HPLC, Whelk O-1 (R,R) from Regis Technology (3μ); i-PrOH 50% = heptane 50% - DEA 0.1%) |
| | or | | |
| | 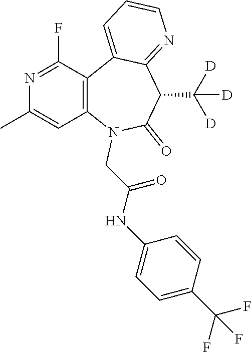 [SMILES: [2H]C([2H])([2H])[C@@H]1C(=O)N(CC(=O)Nc2ccc(C(F)(F)F)cc2)c2cc(C)nc(F)c2-c2cccnc21] | | |
| #204 | 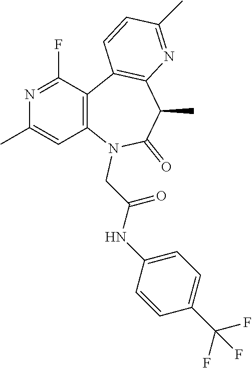 [SMILES: Cc1cc2c(c(F)n1)-c1ccc(C)nc1[C@@H](C)C(=O)N2CC(=O)Nc1ccc(C(F)(F)F)cc1] | Enantiomer (10R) or (10S) of 2-[3-fluoro- 5,10,13-trimethyl-9- oxo-4,8,12- triazatricyclo[9.4.0.0^{2,7}] pentadeca- 1(11),2(7),3,5,12,14- hexaen-8-yl]-N-[4- (trifluoromethyl)phenyl] acetamide | Example #93 was purified by chiral HPLC (column Whelk-O1 (R,R) from Regis Technology, using heptane:iso- propanol (1:1) and 0.1% DEA) to yield compound #204. LC-MS (Method B2) m/z [M + H]⁺: 473.1; rt: 4.90 min; purity: 97%. LC-MS (Method A2) m/z [M + H]⁺: 473.1; rt: 4.91 min; purity: 96%. ¹H NMR (400 MHz, DMSO-d₆) δ 10.59 (s, 1H), 7.99 (dd, J = 8.0, 4.6 Hz, 1H), 7.77 (d, J = 8.8 Hz, 2H), 7.68 (d, J = 8.8 Hz, 2H), 7.40-7.31 (m, 2H), 4.59 (s, 2H), 3.70 (q, J = 6.6 Hz, 1H), 2.55 (s, 3H), 2.50 (s, 3H), 1.47 (d, J = 6.6 Hz, 3H). Chiral purity: 97%; rt = 2.58 min (first eluting enantiomer). For information, second eluting enantiomer rt = 3.75 min. Both measured by HPLC, Whelk O-1 (R,R) from Regis Technology (3μ); i-PrOH 50% - heptane 50% - DEA 0.1%) |
| | or | | |
| | 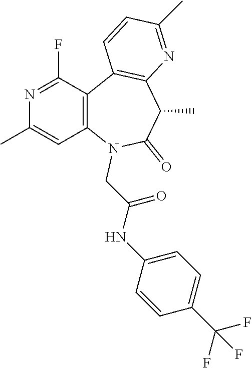 [SMILES: Cc1cc2c(c(F)n1)-c1ccc(C)nc1[C@H](C)C(=O)N2CC(=O)Nc1ccc(C(F)(F)F)cc1] | | |
| #205 | 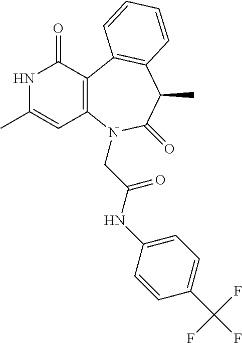 [SMILES: Cc1cc2c(c(=O)[nH]1)-c1ccccc1[C@@H](C)C(=O)N2CC(=O)Nc1ccc(C(F)(F)F)cc1] | Entnaiomer (7R) or (7S) of 2-[3,7-dimethyl- 1,6-dioxo-2,7- dihydropyrido[4,3- d][3]benzazepin-5-yl]- N-[4- (trifluoromethyl)phenyl] acetamide | Example #156 was purified by chiral HPLC (column Whelk-O1 (R,R) from Regis Technology, using heptane:eth- anol (1:1) and 0.1% DEA) to yield compound #205. LC-MS (Method B4) m/z [M + H]⁺: 456.1; rt: 3.78 min; purity: 98%. LC-MS (Method A4) m/z [M + H]⁺: 456.1; rt: 4.16 min; purity: 97%. ¹H NMR (400 MHz, DMSO-d₆) δ 11.82 (s, 1H), 10.57 (s, 1H), 7.86 (dd, J = 7.8, 1.5 Hz, 1H), 7.79 (d, J = 8.5 Hz, 2H), 7.68 (d, J = 8.6 Hz, 2H), 7.40 (td, J = 7.6, 1.5 Hz, 1H), 7.35-7.24 (m, 2H), 6.18 (s, 1H), 4.45-4.31 (m, 2H), 3.41 (q, J = 6.8 Hz, 1H), 2.22 (s, 3H), 1.46 (d, J = 6.8 Hz, 3H). Chiral purity: 94%; rt = 2.17 min (first eluting enantiomer). For information, second eluting enantiomer rt = 4.26 min, measured by HPLC, Whelk O-1 (R,R) from Regis Technology (3μ); |
| | or | | EtOH 50% - heptane 50% - DEA 0.1%). |
| | 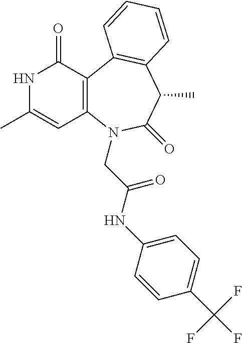 [SMILES: Cc1cc2c(c(=O)[nH]1)-c1ccccc1[C@H](C)C(=O)N2CC(=O)Nc1ccc(C(F)(F)F)cc1] | | |
| #206 | 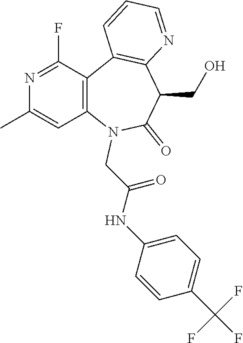 [SMILES: Cc1cc2c(c(F)n1)-c1cccnc1[C@@H](CO)C(=O)N2CC(=O)Nc1ccc(C(F)(F)F)cc1] | Enantiomer (10S) or (10R) of 2-[(3-fluoro- 10-(hydroxymethyl)-5- methyl-9-oxo-4,8,12- triazatricyclo[9.4.0.0^{2,7}] pentadeca- 1(11),2(7),3,5,12,14- hexaen-8-yl]-N-[4- (trifluoromethyl)phenyl] acetamide | Example #152 was purified by chiral HPLC (column chiralpak IC from Daicel, using heptane:isopropanol (1:1) and 0.1% DEA) to yield compound #206. LC-MS (Method B4) m/z [M + H]⁺: 475.1; rt: 3.75 min; purity: 98%. LC-MS (Method A4) m/z [M + H]⁺: 475.1; rt: 4.11 min; purity: 98%. ¹H NMR (400 MHz, DMSO-d₆) δ 10.60 (s, 1H), 8.65 (dd, J = 4.8, 1.7 Hz, 1H), 8.15 (ddd, J = 7.9, 4.6, 1.7 Hz, 1H), 7.75 (d, J = 8.5 Hz, 2H), 7.67 (d, J = 8.7 Hz, 2H), 7.50 (dd, J = 8.0, 4.8 Hz, 1H), 7.44 (s, 1H), 4.69-4.58 (m, 2H), 4.51 (s, 1H), 4.28 (qd, J = 11.2, 7.4 Hz, 2H), 3.64 (dd, J = 7.3, 5.5 Hz, 1H), 2.51 (s, 3H). Chiral purity: ND %; rt = 2.05 min (first eluting enantiomer). For information, second eluting enantiomer rt = 3.69 min. Both measured by HPLC, Chiralpak IC from |
| | or | | Daicel, -i-PrOH 50% - heptane 50% - |
| | 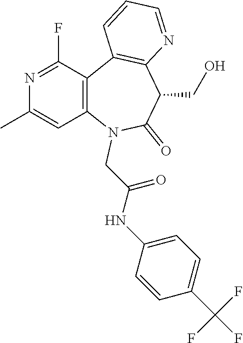 [SMILES: Cc1cc2c(c(F)n1)-c1cccnc1[C@H](CO)C(=O)N2CC(=O)Nc1ccc(C(F)(F)F)cc1] | | DEA 0.1%) |
| #207 | 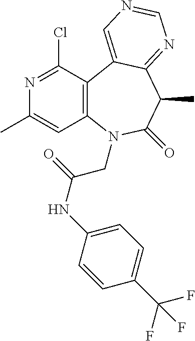 | Enantiomer (8S) or (8R) of 2-[15-chloro- 8,13-dimethyl-9-oxo- 4,6,10,14- tetrazatricyclo[9.4.0.0^{2,7}] pentadeca- 1(11),2,4,6,12,14- hexaen-10-yl]-N-[4- (trifluoromethyl)phenyl] acetamide | Example #163 was purified by chiral SFC (column Chiralpak IA, CO₂ + EtOH 20%) to yield compound #207. LCMS (Method A7): m/z [M + H]⁺: 476.1/478.1; rt: 2.16 min; purity: 95%. ¹H NMR (400 MHz, DMSO-d₆) δ 10.58 (s, 1H), 9.24 (s, 1H), 9.23 (s, 1H), 7.74 (d, J = 8.6 Hz, 2H), 7.67 (d, J = 8.6 Hz, 2H), 7.50 (s, 1H), 4.71-4.48 (m, 2H), 3.96 (q, J = 6.5 Hz, 1H), 2.55 (s, 3H), 1.47 (d, J = 6.6 Hz, 3H). Chiral purity: 99%; rt = 2.77 min (second eluting enantiomer). For information, first eluting enantiomer rt = 1.6 min. Both measured by HPLC, Chiralpak IA from Daicel, EtOH 50% - heptane 50% - DEA 0.1%) |
| | or | | |
| | 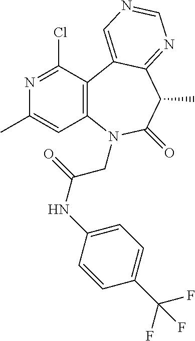 | | |
| #208 | 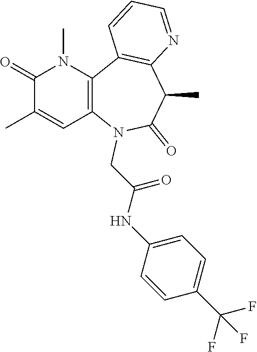 [SMILES: Cc1cc2c(n(C)c1=O)-c1cccnc1[C@@H](C)C(=O)N2CC(=O)Nc1ccc(C(F)(F)F)cc1] | Enantiomer (10S) or (10R) of N-[4- (trifluoromethyl)phenyl]- 2-[3,5,10-trimethyl- 4,9-dioxo-3,8,12- triazatricyclo[9.4.0.0^{2,7}] pentadeca- 1(11),2(7),5,12,14- pentaen-8- yl]acetamide | Example #119 was purified by chiral HPLC (column Chiralpak IF, Ethanol/n-heptane 50/50 (v/v)) to yield compound #208. LC-MS (Method A2′) m/z [M + H]⁺: 471.5; rt: 3.92 min; purity: 99%. Chiral purity: >99%; rt = 3.03 min (second eluting enantiomer). For information, first eluting enantiomer rt = 1.77 min. Both measured by HPLC, Chiralpak IF from Daicel, EtOH 50% - heptane 50% - DEA 0.1%) |
| | or | | |
| | 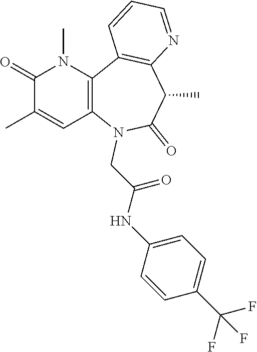 [SMILES: Cc1cc2c(n(C)c1=O)-c1cccnc1[C@H](C)C(=O)N2CC(=O)Nc1ccc(C(F)(F)F)cc1] | | |
| #209 | 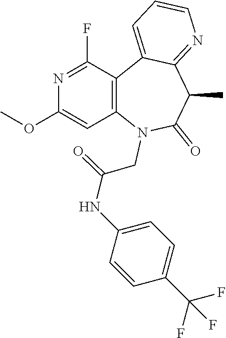 | Enantiomer (10S) or (10R) of 2-[3-fluoro-5- methoxy-10-methyl-9- oxo-4,8,12- triazatricyclo[9.4.0.0^{2,7}] pentadeca- 1(15),2(7),3,5,11,13- hexaen-8-yl]-N-[4- (trifluoromethyl)phenyl] acetamide | Example #182 was purified by chiral SFC (column Chiralcel OD, CO₂ + EtOH 20%) to yield compound #209. LC-MS (Method A2′) m/z [M + H]⁺: 475; rt: 4.87 min; purity: 98% Chiral purity: 99%; rt = 2.78 min (second eluting enantiomer). For information, first eluting enantiomer rt = 1.89 min. Both measured by HPLC, Chiralpak IG-u from Daicel, EtOH 50% - heptane 50% - DEA 0.1%) |
| | or | | |
| | 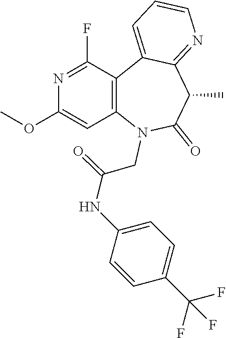 | | |
| #210 | 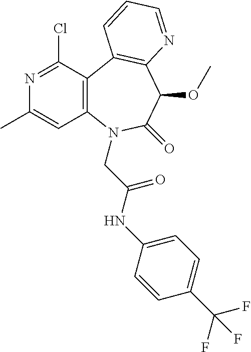 [SMILES: CO[C@H]1C(=O)N(CC(=O)Nc2ccc(C(F)(F)F)cc2)c2cc(C)nc(Cl)c2-c2cccnc21] | Enantiomer (10S) or (10R) of 2-[3-chloro- 10-methoxy-5-methyl- 9-oxo-4,8,12- triazatricyclo[9.4.0.0^{2,7}] pentadeca- 1(11),2(7),3,5,12,14- hexaen-8-yl]-N-[4- (trifluoromethyl)phenyl] acetamide | Example #159 was purified by chiral SFC (column Chiralpak IG-CO2 + EtOH 20%) to yield compound #210. LC-MS (Method A2′) [M + H]⁺: 491, rt; 4.20 min; purity: 98%. Chiral purity: 99%; rt = 1.75 min (second eluting enantiomer). For information, first eluting enantiomer rt = 1.47 min. Both measured by HPLC, Chiralpak IB from Daicel, ACN 100% - DEA 0.1%) |
| | or | | |
| | 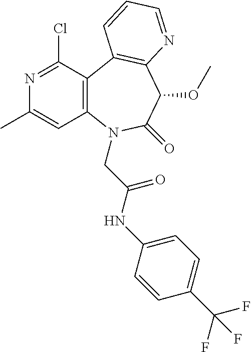 [SMILES: CO[C@@H]1C(=O)N(CC(=O)Nc2ccc(C(F)(F)F)cc2)c2cc(C)nc(Cl)c2-c2cccnc21] | | |
| #211 | 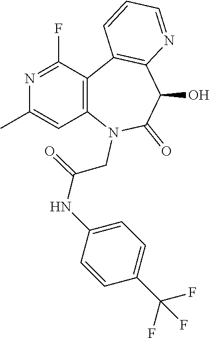 [SMILES: Cc1cc2c(c(F)n1)-c1cccnc1[C@@H](O)C(=O)N2CC(=O)Nc1ccc(C(F)(F)F)cc1] | Enantiomer (10S) or (10R) of 2-[3-fluoro-10- hydroxy-5-methyl-9- oxo-4,8,12- triazatricyclo[9.4.0.0^{2,7}] pentadeca- 1(11),2(7),3,5,12,14- hexaen-8-yl]-N-[4- (trifluoromethyl)phenyl] acetamide | Example #94 was purified by chiral SFC (column Chiralpak AD-CO₂ + EtOH 30%) to yield compound #211. LC-MS (Method A2′) as a mixture of two conformer (ratio 95.7/3.4), m/z [M + H]⁺: 461, rt: 3.91 min (purity: 95.7%); 4.06 min (purity: 3.4%). Chiral purity: 95%; rt = 1.92 min (first eluting enantiomer). For information, second eluting enantiomer rt = 3.17 min. Both measured by HPLC, Chiralpak IB from Daicel, EtOH 100% - DEA 0.1%) |
| | or | | |
| | 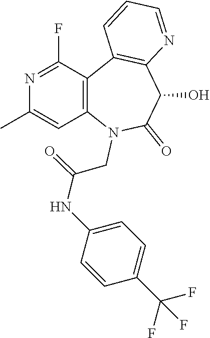 [SMILES: Cc1cc2c(c(F)n1)-c1cccnc1[C@H](O)C(=O)N2CC(=O)Nc1ccc(C(F)(F)F)cc1] | | |

### Example #212: 2-(3,12-difluoro-5-methyl-9-oxo-4,8,14-triazatricyclo[9.4.0.0^{2,7}]pentadeca-1(11),2(7),3,5,12,14-hexaen-8-yl)-N-[4-(trifluoromethyl)phenyl]acetamide

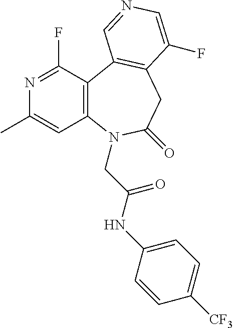 [SMILES: Cc1cc2c(c(F)n1)-c1cncc(F)c1CC(=O)N2CC(=O)Nc1ccc(C(F)(F)F)cc1]

The title product was prepared according to general procedure 1, starting from 3,12-difluoro-5-methyl-4,8,14-triazatricyclo[9.4.0.0^{2,7}]pentadeca-1(11),2(7),3,5,12,14-hexaen-9-one (Intermediate N65) and 2-chloro-N-(4-trifluoromethyl-phenyl)acetamide. Purification by flash chromatography on silica gel (using a gradient of 0 to 10% MeOH in DCM as eluent) and trituration in MTBE gave the title compound (61 mg, yield: 56%) as a beige solid. LC-MS (Method A7) m/z: [M+H]⁺: 463; rt: 2.14 min; purity: 99%. ¹H NMR (400 MHz, DMSO-d₆) δ 10.62 (s, 1H), 8.79 (d, J=5.3 Hz, 1H), 8.71 (s, 1H), 7.77 (d, J=8.5 Hz, 2H), 7.68 (d, J=8.7 Hz, 2H), 7.40 (s, 1H), 4.60 (s, 2H), 3.91 (d, J=13.1 Hz, 1H), 3.62-3.56 (m, 1H) 2.51 (s, 3H).

### Example #213: Enantiomer (10S) or (10R) of N-(4-bromo-3-chloro-phenyl)-2-[(10S)-3-fluoro-10-hydroxy-5-methyl-9-oxo-4,8,12-triazatricyclo[9.4.0.0^{2,7}]pentadeca-1(11),2(7),3,5,12,14-hexaen-8-yl]acetamide

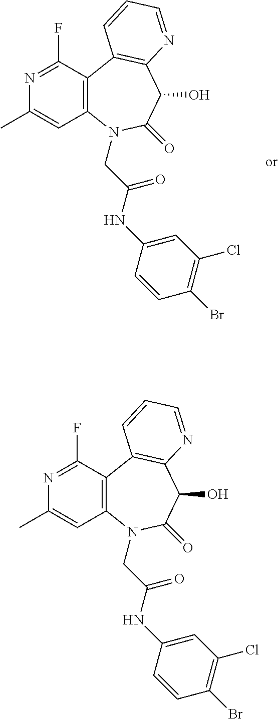 [SMILES: Cc1cc2c(c(F)n1)-c1cccnc1[C@H](O)C(=O)N2CC(=O)Nc1ccc(Br)c(Cl)c1 ; Cc1cc2c(c(F)n1)-c1cccnc1[C@@H](O)C(=O)N2CC(=O)Nc1ccc(Br)c(Cl)c1]

The title product was prepared according to the General Procedure 1 starting from 3-fluoro-10-hydroxy-5-methyl-4,8,12-triazatricyclo[9.4.0.0^{2,7}]pentadeca-1(11),2(7),3,5,12,14-hexaen-9-one N22 (327.7 mg, 1.138 mmol), and N-(4-bromo-3-chloro-phenyl)-2-chloro-acetamide (CAS: 22521-43-3, 431 mg, 1.478 mmol). After purification by SFC (Kromasil SFC-5-Diol from Kromasil, eluant: CO₂+EtOH 25%), the racemate was separated by chiral HPLC (Chiralcel OD from Daicel, eluant: EtOH 100%—DEA 0.1%). The second eluting enantiomer was repurified by SFC (SFC 2-Ethylpyridine 60A 5p from Princeton, eluent: CO₂+EtOH 20%) to afford the title compound as a white solid (17.6 mg, yield 3%). Chiral purity: 97%; rt=2.41 min. For information, first eluting enantiomer rt=1.58 min. Both measured by HPLC (Chiralcel OD from Daicel, eluant: EtOH 100%—DEA 0.1%). LC-MS (Method B2) m/z [M+H]⁺: 504.8/506.9/508.8; rt: 4.13 min; purity: 91%. LC-MS m/z [M+H]⁺: 504.9/506.9/508.9; purity: 87%. ¹H NMR (400 MHz, DMSO-d₆) δ 10.56 (s, 1H), 8.71 (dd, J=4.8, 1.6 Hz, 1H), 8.21-8.14 (m, 1H), 7.96 (d, J=2.4 Hz, 1H), 7.70 (d, J=8.8 Hz, 1H), 7.57 (dd, J=7.9, 4.8 Hz, 1H), 7.40-7.34 (m, 2H), 5.69 (d, J=8.6 Hz, 1H), 5.16 (d, J=8.6 Hz, 1H), 4.58 (s, 2H) (+one CH₃ present under the signal of the DMSO). ¹⁹F NMR (376 MHz, DMSO-d₆) δ−70.55 (d, J=4.4 Hz).

### Example #214: Enantiomer (10S) or (10R) of N-[4-(difluoromethyl)phenyl]-2-[3-fluoro-5-methoxy-10-methyl-9-oxo-4,8,12-triazatricyclo[9.4.0.0^{2,7}]pentadeca-1(15),2(7),3,5,11,13-hexaen-8-yl]acetamide

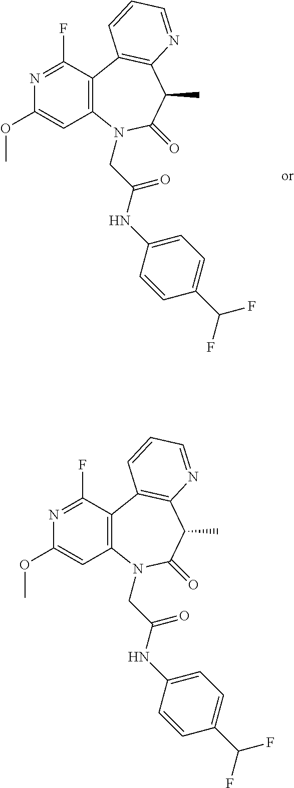 [SMILES: COc1cc2c(c(F)n1)-c1cccnc1[C@@H](C)C(=O)N2CC(=O)Nc1ccc(C(F)F)cc1 ; COc1cc2c(c(F)n1)-c1cccnc1[C@H](C)C(=O)N2CC(=O)Nc1ccc(C(F)F)cc1]

The title product was prepared according to the General Procedure 1, starting from intermediate (10R)-3-fluoro-5-methoxy-10-methyl-4,8,12-triazatricyclo[9.4.0.0^{2,7}]pentadeca-1(11),2(7),3,5,12,14-hexaen-9-one N58-A (750 mg, 2.7 mmol) and 2-chloro-N-[4-(difluoromethyl)phenyl]acetamide (660 mg, 3 mmol). The obtained solid was taken up in EtOAc, filtered off and rinsed with EtOAc to afford 759 mg of white solid (61% yield). LC-MS m/z [M+H]⁺: 457.1; purity: 99%. ¹H NMR (500 MHz, DMSO-d₆) δ 10.48 (s, 1H), 8.64 (dd, J=4.7, 1.6 Hz, 1H), 8.09 (ddd, J=8.0, 4.4, 1.6 Hz, 1H), 7.68 (d, J=8.3 Hz, 2H), 7.54-7.46 (m, 3H), 6.97 (t, 56.4 Hz, 1H), 6.91 (s, 1H), 4.60 (s, 2H), 3.92 (s, 3H), 3.85 (q, J=6.5 Hz, 1H), 1.48 (d, J=6.6 Hz, 3H). Chiral purity: 99%; rt=2.3 min (second eluting enantiomer). For information, first eluting enantiomer rt=1.6 min. Both measured by chiral HPLC with column CHIRALCEL OD-3 (4.6×150 mm) from Daicel, eluent MeOH 100%+0.1% DEA, at 1.5 mL/min and 30° C.

### Example #215: Enantiomer (10S) or (10R) of N-(4-chloro-3-fluoro-phenyl)-2-[3-fluoro-5-methoxy-10-methyl-9-oxo-4,8,12-triazatricyclo[9.4.0.0^{2,7}]pentadeca-1(15),2(7),3,5,11,13-hexaen-8-yl]acetamide

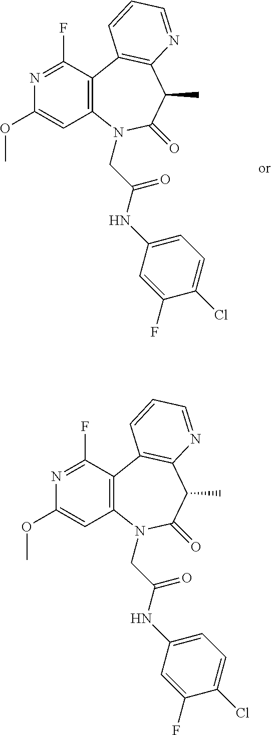 [SMILES: COc1cc2c(c(F)n1)-c1cccnc1[C@@H](C)C(=O)N2CC(=O)Nc1ccc(Cl)c(F)c1 ; COc1cc2c(c(F)n1)-c1cccnc1[C@H](C)C(=O)N2CC(=O)Nc1ccc(Cl)c(F)c1]

The title product was prepared according to the general procedure (1), starting from intermediate (10R)-3-fluoro-5-methoxy-10-methyl-4,8,12-triazatricyclo[9.4.0.0^{2,7}]pentadeca-1(11),2(7),3,5,12,14-hexaen-9-one N58_A (500 mg, 2 mmol) and 2-chloro-N-(4-chloro-3-fluoro-phenyl)acetamide (447 mg, 2.01 mmol). The obtained solid was taken up in EtOAc, filtered off and rinsed with EtOAc to afford 300 mg of a white solid (36% yield). LC-MS m/z [M+H]⁺: 459.1; rt: 4.73 min; purity: 98%. ¹H NMR (500 MHz, DMSO-d₆) δ 10.60 (s, 1H), 8.64 (dd, J=4.7, 1.6 Hz, 1H), 8.09 (ddd, J=7.9, 4.4, 1.6 Hz, 1H), 7.72 (dd, J=11.8, 2.4 Hz, 1H), 7.56-7.45 (m, 2H), 7.33-7.27 (m, 1H), 6.90 (s, 1H), 4.58 (s, 2H), 3.92 (s, 3H), 3.85 (q, J=6.6 Hz, 1H), 1.48 (d, J=6.6 Hz, 3H).

### Example #216: Enantiomer (10S) or (10R) of N-(3-chloro-4-fluoro-phenyl)-2-(3-fluoro-5-methoxy-10-methyl-9-oxo-4,8,12-triazatricyclo[9.4.0.0^{2,7}]pentadeca-1(11),2(7),3,5,12,14-hexaen-8-yl)acetamide

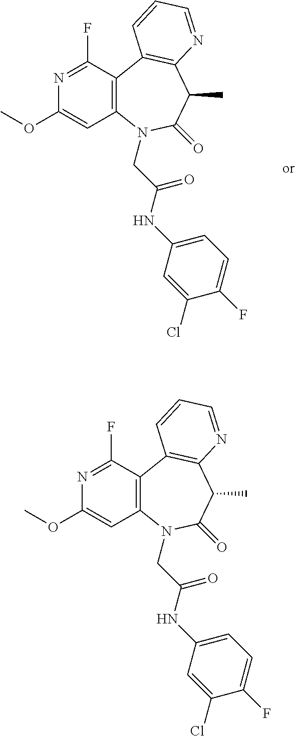 [SMILES: COc1cc2c(c(F)n1)-c1cccnc1[C@@H](C)C(=O)N2CC(=O)Nc1ccc(F)c(Cl)c1 ; COc1cc2c(c(F)n1)-c1cccnc1[C@H](C)C(=O)N2CC(=O)Nc1ccc(F)c(Cl)c1]

To a solution of Enantiomer (10S) or (10R) of 2-(3-fluoro-5-methoxy-10-methyl-9-oxo-4,8,12-triazatricyclo[9.4.0.0^{2,7}]pentadeca-1(11),2(7),3,5,12,14-hexaen-8-yl)acetic acid (Intermediate NN155, 60.0 mg, 0.15 mmol) and 3-chloro-4-fluoroaniline (42.2 mg, 0.29 mmol) in MeCN (1.5 mL) were added TCFH (83.0 mg, 0.29 mmol) and 1-methylimidazole (46.7 μL, 0.58 mmol) under N₂ atmosphere. The resulting mixture was stirred at room temperature for 1 h. After complete conversion, water (30 mL) was added and the reaction mixture was extracted with EtOAc (3×30 mL). The combined organic extracts were dried over MgSO₄, filtered, and concentrated under vacuum. The residue was purified by reverse phase flash chromatography to afford the title compound (59 mg, yield: 87%) as a white solid. LC-MS m/z: [M+H]⁺: 459.1/460.9; rt: 4.70 min; purity: 98%. ¹H NMR (400 MHz, DMSO-d₆) δ 10.49 (s, 1H), 8.64 (dd, J=4.8, 1.7 Hz, 1H), 8.09 (ddd, J=7.9, 4.5, 1.7 Hz, 1H), 7.88 (dd, J=6.8, 2.4 Hz, 1H), 7.49 (dd, J=7.9, 4.8 Hz, 1H), 7.45-7.33 (m, 2H), 6.89 (s, 1H), 4.56 (s, 2H), 3.92 (s, 3H), 3.84 (q, J=6.6 Hz, 1H), 1.48 (d, J=6.6 Hz, 3H). ¹⁹F NMR (376 MHz, DMSO-d₆) δ−72.08 (d, J=4.5 Hz), −122.63 (ddd, J=8.9, 6.8, 4.8 Hz).

### Example #217: Enantiomer (8S) or (8R) of N-(4-chloro-3-fluoro-phenyl)-2-[15-fluoro-8,13-dimethyl-9-oxo-4,6,10,14-tetrazatricyclo[9.4.0.0^{2,7}]pentadeca-1(15),2,4,6,11,13-hexaen-10-yl]acetamide

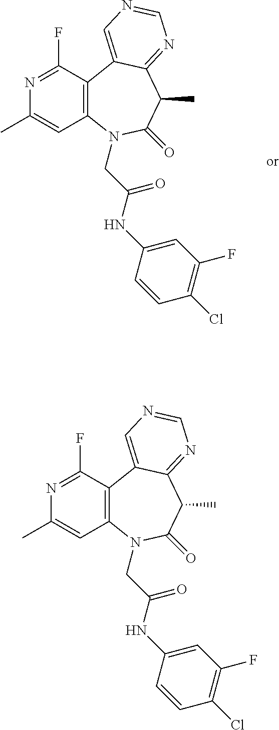 [SMILES: Cc1cc2c(c(F)n1)-c1cncnc1[C@@H](C)C(=O)N2CC(=O)Nc1ccc(Cl)c(F)c1 ; Cc1cc2c(c(F)n1)-c1cncnc1[C@H](C)C(=O)N2CC(=O)Nc1ccc(Cl)c(F)c1]

The title product was prepared according to the general procedure (1), starting from intermediate 15-fluoro-8,13-dimethyl-4,6,10,14-tetrazatricyclo[9.4.0.0^{2,7}]pentadeca-1(15),2(7),3,5,11,13-hexaen-9-one N36 and 2-chloro-N-(4-chloro-3-fluoro-phenyl)acetamide. The solid (324 mg) was separated by chiral SFC with column Chiralpak IA 20 μm (50×266 mm) from Daicel, eluent CO₂+25% EtOH, at 360 mL/min, 30° C. and back pressure 150 bar to yield the pure enantiomer as a white solid (105 mg, yield 32%). Chiral purity: 100%; rt=2.25 min (second eluting enantiomer). For information, first eluting enantiomer rt=1.66 min. Both measured by chiral HPLC with column CHIRALPAK IA-3 (4.6×150 mm) from Daicel, eluent EtOH:n-heptane 1:1+0.1% DEA, at 1.5 mL/min and 30° C. LC-MS m/z [M+H]⁺: 444.0; purity: 97%. ¹H NMR (400 MHz, DMSO-d₆) δ 10.58 (s, 1H), 9.26 (s, 1H), 9.13 (d, J=4.6 Hz, 1H), 7.72 (dd, J=11.8, 2.4 Hz, 1H), 7.52 (t, J=8.7 Hz, 1H), 7.41 (s, 1H), 7.37-7.27 (m, 1H), 4.59 (s, 2H), 3.92 (q, J=6.5 Hz, 1H), 2.5 (s, 3H, hidden by DMSO-d₆), 1.48 (d, J=6.6 Hz, 3H).

### Example #218: Enantiomer (8S) or (8S) of (N-[4-(difluoromethyl)phenyl]-2-[15-fluoro-8,13-dimethyl-9-oxo-4,6,10,14-tetrazatricyclo[9.4.0.0^{2,7}]pentadeca-1(15),2,4,6,11,13-hexaen-10-yl]acetamide

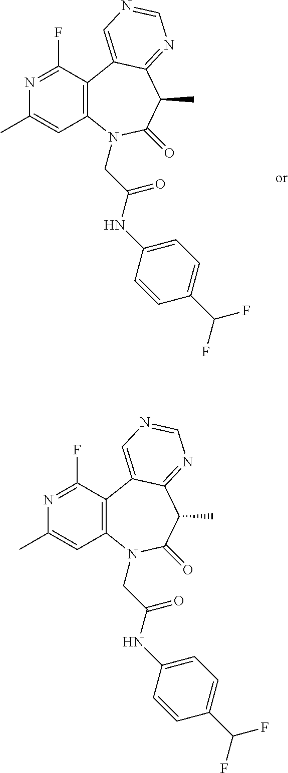 [SMILES: Cc1cc2c(c(F)n1)-c1cncnc1[C@@H](C)C(=O)N2CC(=O)Nc1ccc(C(F)F)cc1 ; Cc1cc2c(c(F)n1)-c1cncnc1[C@H](C)C(=O)N2CC(=O)Nc1ccc(C(F)F)cc1]

The title product was prepared according to the general procedure 1, starting from 15-fluoro-8,13-dimethyl-4,6,10,14-tetrazatricyclo[9.4.0.0^{2,7}]pentadeca-1(15),2(7),3,5,11,13-hexaen-9-one N36 (165 mg, 0.51 mmol) and 2-chloro-N-[4-(difluoromethyl)phenyl]acetamide (CAS 872533-93-2). After purification by column chromatography, the racemate was separated by Chiral SFC (column CHIRALPAK IA from Daicel, eluant 35% MeOH+0.1% NH₃, 65% CO₂) to yield the pure enantiomer as a white solid (23 mg, yield: 10%). LC-MS (Method 2) m/z [M+H]⁺: 442.0; rt: 1.73 min; purity: 100%. ¹H NMR (400 MHz, DMSO-d₆) δ 10.47 (s, 1H), 9.26 (s, 1H), 9.14 (d, J=4.6 Hz, 1H), 7.68 (d, J=8.4 Hz, 2H), 7.51 (d, J=8.3 Hz, 2H), 7.42 (s, 1H), 6.96 (t, J=56.1 Hz, 1H), 4.61 (s, 2H), 3.92 (q, J=6.5 Hz, 1H), 2.52 (s, 3H), 1.48 (d, J=6.6 Hz, 3H). ¹⁹F NMR (376 MHz, DMSO-d₆) δ−71.17, −108.12. Chiral purity: 99%; rt=2.56 min (second eluting enantiomer). For information, first eluting enantiomer rt=1.70 min. Both measured by SFC, CHIRALPAK IA from Daicel, 30% MeOH+0.1% NH₃, 70% CO₂.

### Example #219: Enantiomer (7S) or (7R) of N-(4-chloro-3-fluoro-phenyl)-2-[2,3,7-trimethyl-1,6-dioxo-7H-pyrido[4,3-d][3]benzazepin-5-yl]acetamide

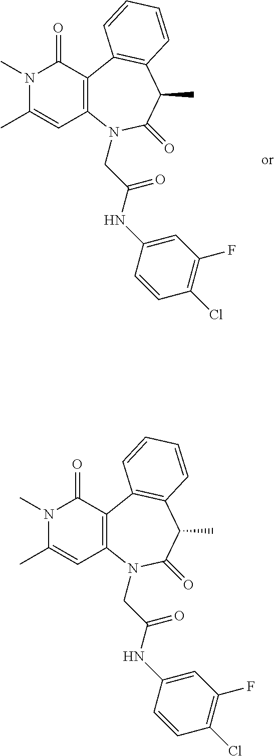 [SMILES: Cc1cc2c(c(=O)n1C)-c1ccccc1[C@@H](C)C(=O)N2CC(=O)Nc1ccc(Cl)c(F)c1 ; Cc1cc2c(c(=O)n1C)-c1ccccc1[C@H](C)C(=O)N2CC(=O)Nc1ccc(Cl)c(F)c1]

The title compound was prepared according to the procedure described for example #128, starting from [2-(2,3,7-trimethyl-1,6-dioxo-7H-pyrido[4,3-d][3]benzazepin-5-yl)acetyl]oxylithium N109 and 4-chloro-3-fluoro-aniline. The solid (racemate, 121 mg) was separated by chiral SFC with column Chiralpak AD 20 μm (50×279 mm) from Daicel, eluent CO₂₊₂₅% EtOH, at 360 mL/min, 30° C. and back pressure 150 bar to yield the pure enantiomer as a white solid (38 mg, yield: 31%). Chiral purity: 99%; rt=3.38 min (second eluting enantiomer). For information, first eluting enantiomer rt=2.33 min. Both measured by chiral HPLC with column CHIRALPAK AD-3 (4.6×150 mm) from Daicel, eluent EtOH 100%+0.1% DEA, at 1.5 mL/min and 30° C. LC-MS m/z [M+H]⁺: 454.0; purity: 98%. ¹H NMR (400 MHz, DMSO-d₆) δ 10.50 (s, 1H), 7.83 (dd, J=7.8, 1.4 Hz, 1H), 7.75 (dd, J=11.9, 2.4 Hz, 1H), 7.52 (t, J=8.7 Hz, 1H), 7.40 (td, J=7.6, 1.4 Hz, 1H), 7.34-7.24 (m, 3H), 6.33 (s, 1H), 4.43-4.29 (m, 2H), 3.50 (s, 3H), 3.45-3.38 (m, 1H), 2.42 (s, 3H), 1.45 (d, J=6.8 Hz, 3H).

### Example #220: Enantiomer (8S) or (8R) of 2-[15-chloro-8,13-dimethyl-9-oxo-4,6,10,14-tetrazatricyclo[9.4.0.0^{2,7}]pentadeca-1(15),2,4,6,11,13-hexaen-10-yl]-N-(4-chloro-3-fluoro-phenyl)acetamide

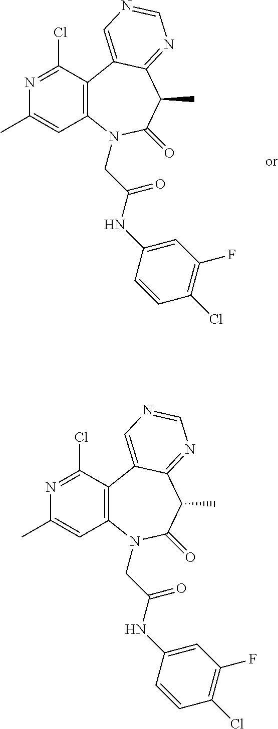 [SMILES: Cc1cc2c(c(Cl)n1)-c1cncnc1[C@@H](C)C(=O)N2CC(=O)Nc1ccc(Cl)c(F)c1 ; Cc1cc2c(c(Cl)n1)-c1cncnc1[C@H](C)C(=O)N2CC(=O)Nc1ccc(Cl)c(F)c1]

A mixture of N-(4-chloro-3-fluoro-phenyl)-2-(15-fluoro-8,13-dimethyl-9-oxo-4,6,10,14-tetrazatricyclo[9.4.0.02,7]pentadeca-1(15),2(7),3,5,11,13-hexaen-10-yl)acetamide #217 (racemic mixture, 0.45 g, 0.97 mmol) and HCl (4N in 1,4-dioxane) (5 mL, 20.0 mmol) was heated at 100° C. in a sealed microwave vial for 90 minutes. The reaction mixture was allowed to cool to RT and then the crude reaction mixture carefully poured onto saturated NaHCO₃ solution (30 mL). The product was extracted into EtOAc (3×20 mL) and the organics were combined, dried over Na₂SO₄, filtered and concentrated under vacuo. The crude product was purified by column chromatography on silica gel. The solid (racemate, 245 mg) was separated by chiral SFC with column Chiralpak IF 20 μm (50×279 mm) from Daicel, eluent CO₂+20% EtOH, at 360 mL/min, 30° C. and BPR 150 bar to yield the pure enantiomer as a white solid (97 mg, yield: 40%). Chiral purity: 100%; rt=4.55 min (second eluting enantiomer). For information, first eluting enantiomer rt=1.66 min. Both measured by chiral HPLC with column CHIRALPAK IF-3 (4.6×150 mm) from Daicel, eluent EtOH:n-heptane 1:1+0.1% DEA, at 1.5 mL/min and 30° C. LC-MS m/z [M+H]⁺: 460.0; purity: 99%. ¹H NMR (400 MHz, DMSO-d₆) δ 10.55 (s, 1H), 9.24 (s, 1H), 9.23 (s, 1H), 7.70 (d, J=11.9 Hz, 1H), 7.56-7.46 (m, 2H), 7.28 (d, J=9.1 Hz, 1H), 4.56 (q, J=16.8 Hz, 2H), 4.00-3.90 (m, 1H), 2.55 (s, 3H), 1.47 (d, J=6.5 Hz, 3H).

### Example #221: Enantiomer (7S) or (7R) of N-[4-(difluoromethyl)phenyl]-2-[14-fluoro-7,12-dimethyl-8-oxo-5,6,9,13-tetrazatricyclo[8.4.0.0^{2,6}]tetradeca-1(14),2,4,10,12-pentaen-9-yl]acetamide

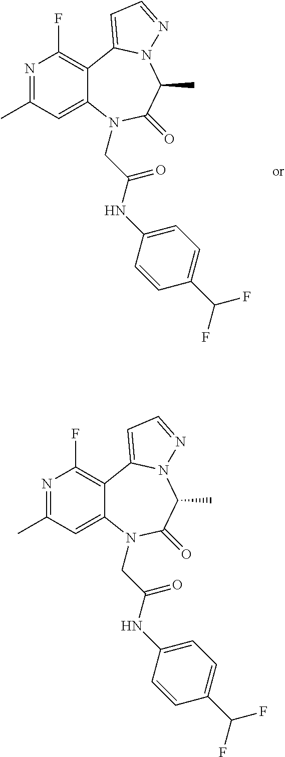 [SMILES: Cc1cc2c(c(F)n1)-c1ccnn1[C@@H](C)C(=O)N2CC(=O)Nc1ccc(C(F)F)cc1 ; Cc1cc2c(c(F)n1)-c1ccnn1[C@H](C)C(=O)N2CC(=O)Nc1ccc(C(F)F)cc1]

The title product was prepared according to the general procedure 1, starting from 14-fluoro-7,12-dimethyl-5,6,9,13-tetrazatricyclo[8.4.0.0^{2,6}]tetradeca-1(10),2,4,11,13-pentaen-8-one N35 (145 mg, 0.42 mmol) and 2-chloro-N-[4-(difluoromethyl)phenyl]acetamide (CAS 872533-93-2). The crude product was purified by reversed phase HPLC (acidic elution). The racemate was separated by Chiral SFC (column Lux A1, from Phenomenex, eluant 55% MeOH+0.1% NH₃ 45% CO₂) to yield the pure enantiomer as a white solid (33 mg, yield: 18%). LC-MS m/z [M+H]⁺: 430.0; purity: 100%. ¹H NMR (400 MHz, DMSO-d₆) δ 10.52 (s, 1H), 7.78-7.58 (m, 3H), 7.52 (d, J=8.3 Hz, 2H), 7.38 (s, 1H), 6.97 (t, J=56.1 Hz, 1H), 6.75 (dd, J=3.6, 2.0 Hz, 1H), 5.00 (q, J=6.6 Hz, 1H), 4.72-4.49 (m, 2H), 2.49 (s, 3H), 1.71 (d, J=6.7 Hz, 3H)¹⁹F NMR (376 MHz, DMSO-d₆) δ−68.77, −108.13. Chiral purity: 100%; rt=4.26 min (second eluting enantiomer). For information, first eluting enantiomer rt=1.17 min. Both measured by SFC, Lux A1 from Phenomenex, 55% CO₂ and 45% MeOH+0.1% NH₃.

### Example #222: Enantiomer (10S) or (10R) of N-[4-(difluoromethyl)phenyl]-2-[3-fluoro-10-(hydroxymethyl)-5-methyl-9-oxo-4,8,12-triazatricyclo[9.4.0.0^{2,7}]pentadeca-1(15),2(7),3,5,11,13-hexaen-8-yl]acetamide

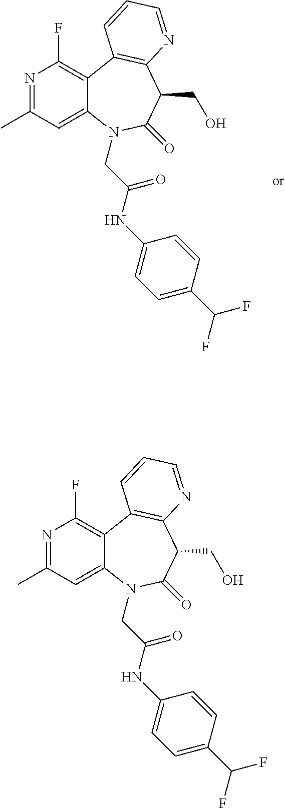 [SMILES: Cc1cc2c(c(F)n1)-c1cccnc1[C@@H](CO)C(=O)N2CC(=O)Nc1ccc(C(F)F)cc1 ; Cc1cc2c(c(F)n1)-c1cccnc1[C@H](CO)C(=O)N2CC(=O)Nc1ccc(C(F)F)cc1]

To a stirred solution of N-[4-(difluoromethyl)phenyl]-2-[3-fluoro-5-methyl-9-oxo-10-(2-trimethylsilylethoxymethyl)-4,8,12-triazatricyclo[9.4.0.0^{2,7}]pentadeca-1(11),2,4,6,12,14-hexaen-8-yl]acetamide N75, prepared according to a similar procedure as the one described for example #152 starting from N75 and 2-chloro-N-(4-chloro-3-fluoro-phenyl)acetamide, (130 mg, 0.23 mmol) in DCM (7 mL) was added TFA (0.85 mL, 11.4 mmol) and the resulting solution was stirred at RT for 30 hours. The reaction mixture was diluted with DCM (40 mL) and transferred to separation a funnel and washed with sat. aqueous NaHCO₃ solution (2×60 mL). The resulting DCM solution was dried over anhydrous Na₂SO₄ and filtered and concentrated under reduced pressure. After purification by column chromatography, the racemate was separated by Chiral SFC (column CHIRALPAK IA from Daicel, eluant 25% MeOH+0.03% NH₃, 75% CO₂) to yield the pure enantiomer as a white solid (27 mg, yield: 43%). LC-MS m/z [M+H]⁺: 457.1; purity: 91%. ¹H NMR (400 MHz, DMSO-d₆) δ 10.46 (s, 1H), 8.64 (dd, J=4.8, 1.7 Hz, 1H), 8.23-8.06 (m, 1H), 7.67 (d, J=8.4 Hz, 2H), 7.59-7.47 (m, 3H), 7.44 (s, 1H), 7.03 (t, J=56.1 Hz, 1H), 4.63 (d, J=2.7 Hz, 2H), 4.51 (s, 1H), 4.37-4.19 (m, 2H), 3.64 (dd, J=7.2, 5.6 Hz, 1H). CH₃ proton signal merged with DMSO solvent residual signal. ¹⁹F NMR (376 MHz, DMSO-d₆) δ−70.81, −108.11. Chiral purity: 98%; rt=4.16 min (second eluting enantiomer). For information, first eluting enantiomer rt=2.99 min. Both measured by SFC, A1 from Phenomenex, 35% MeOH+0.1% NH₃, 65% CO₂.

### Example #223: Enantiomer (10R) or (10S) of 2-[3-chloro-10-fluoro-5-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide

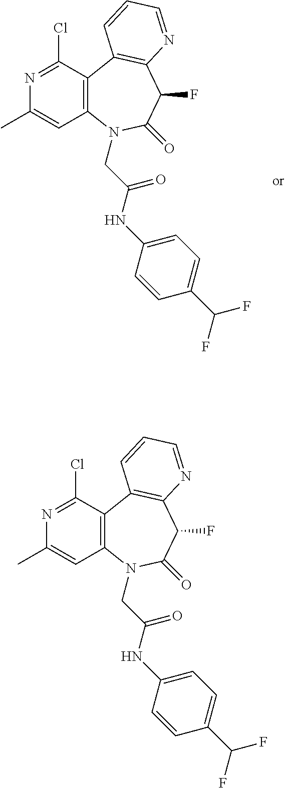 [SMILES: Cc1cc2c(c(Cl)n1)-c1cccnc1[C@@H](F)C(=O)N2CC(=O)Nc1ccc(C(F)F)cc1 ; Cc1cc2c(c(Cl)n1)-c1cccnc1[C@H](F)C(=O)N2CC(=O)Nc1ccc(C(F)F)cc1]

The title product was prepared according to the general procedure 1, starting from intermediate N60 (350 mg, 0.63 mmol) and 2-chloro-N-[4-(difluoromethyl)phenyl]acetamide. Purification by column chromatography on silica gel (using a gradient of 20% to 100% EtOAc in heptane as eluent), followed by trituration in diethyl ether afforded the title racemic compound as a white solid (230 mg, yield: 40%). The racemate was separated by SFC (column Chiralpak OD-I from Daicel 25% Ethanol+75% CO₂ at 35° C.) and repurified by reverse phase chromatography (basic elution) to yield the pure enantiomer as a white solid (67 mg, yield: 19%). LC-MS (Method B3) m/z [M+H]⁺: 461.1; rt: 3.50 min; purity: 97%. LC-MS m/z [M+H]⁺: 461.0; purity: 97%. ¹H NMR (400 MHz, DMSO-d₆) δ 10.48 (s, 1H), 8.71 (s, 1H), 8.30 (d, J=7.9 Hz, 1H), 7.67 (d, J=8.4 Hz, 2H), 7.63-7.55 (m, 1H), 7.55-7.46 (m, 3H), 6.96 (t, J=56.2 Hz, 1H), 6.35 (d, J=45.6 Hz, 1H), 4.67 (d, J=16.8 Hz, 1H), 4.57 (d, J=16.8 Hz, 1H), 2.54 (s, 3H). Chiral purity>99%; rt=2.35 min (second eluting enantiomer). For information, first eluting enantiomer rt=1.86 min. Both measured by HPLC, Chiralpak IB-3 from Daicel, EtOH 50%—heptane 50%—DEA 0.1%.

### Example #224: Enantiomer (10S) or (10R) of N-(4-bromo-3-fluoro-phenyl)-2-[3-chloro-5,10-dimethyl-9-oxo-4,8,12-triazatricyclo[9.4.0.0^{2,7}]pentadeca-1(15),2,4,6,11,13-hexaen-8-yl]acetamide

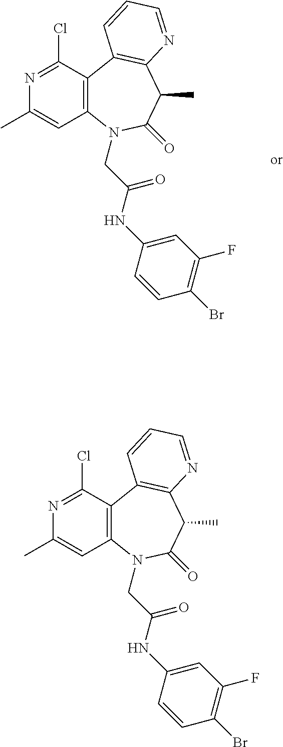 [SMILES: Cc1cc2c(c(Cl)n1)-c1cccnc1[C@@H](C)C(=O)N2CC(=O)Nc1ccc(Br)c(F)c1 ; Cc1cc2c(c(Cl)n1)-c1cccnc1[C@H](C)C(=O)N2CC(=O)Nc1ccc(Br)c(F)c1]

The title product was prepared according to the general procedure 1, starting from (7R) or (7S)-1-chloro-3,7-dimethyl-5,7-dihydrodipyrido[2,3-c:3′,4′-f]azepin-6-one N31_A (100 mg, 0,3654 mmol) and N-(4-bromo-3-fluoro-phenyl)-2-chloro-acetamide (CAS: 1521715-10-5, 116.8 mg, 0,4384 mmol). The residue was purified by preparative HPLC (SFAR_C18_30 g cartridge using NH₄HCO₃ 0.01M and NH₄OH 0.001M NH₄OH in water/MeCN (30 to 100%) as eluent) to give the title compound (118 mg, yield: 64%) as a white solid. LC-MS m/z: [M+H]⁺: 503/505; purity: 94.5%. ¹H NMR (400 MHz, DMSO-d₆) δ 10.55 (s, 1H), 8.65 (dd, J=4.8, 1.7 Hz, 1H), 8.23 (dd, J=7.9, 1.7 Hz, 1H), 7.69 (dd, J=11.4, 2.4 Hz, 1H), 7.63 (t, J=8.4 Hz, 1H), 7.48 (dd, J=7.9, 4.8 Hz, 1H), 7.44 (s, 1H), 7.25 (dd, J=8.8, 2.4 Hz, 1H), 4.60-4.46 (m, 2H), 3.80 (q, J=6.5 Hz, 1H), 2.53 (s, 3H), 1.48 (d, J=6.6 Hz, 3H). The chirality was assessed by chiral HPLC (Chiralpak IG-u from Daicel, EtOH 50%-Heptane 50%-DEA 0.1%; rt 3.48 min; purity: 98.5%)

### Example #225: Enantiomer (10S) or (10R) of N-[2-fluoro-4-(trifluoromethyl)phenyl]-2-[3-chloro-5,10-dimethyl-9-oxo-4,8,12-triazatricyclo[9.4.0.0^{2,7}]pentadeca-1(11),2(7),3,5,12,14-hexaen-8-yl]acetamide

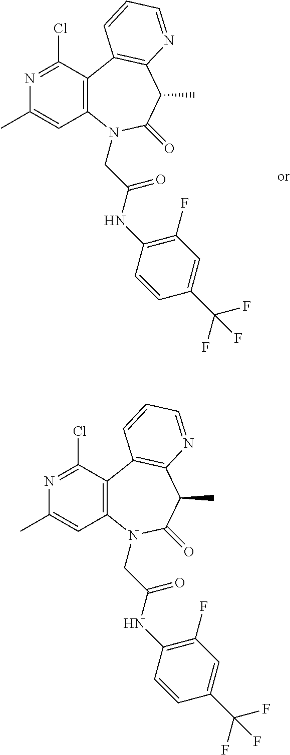 [SMILES: Cc1cc2c(c(Cl)n1)-c1cccnc1[C@H](C)C(=O)N2CC(=O)Nc1ccc(C(F)(F)F)cc1F ; Cc1cc2c(c(Cl)n1)-c1cccnc1[C@@H](C)C(=O)N2CC(=O)Nc1ccc(C(F)(F)F)cc1F]

To a solution of (10S) or (10R)-2-[3-chloro-5,10-dimethyl-9-oxo-4,8,12-triazatricyclo[9.4.0.0^{2,7}]pentadeca-1(11),2(7),3,5,12,14-hexaen-8-yl]acetic acid hydrochloride NN155a (60.0 mg, 0.163 mmol) and 4-amino-3-fluorobenzotrifluoride (61.5 mg, 0.326 mmol) in MeCN (1.6 mL) were added, at room temperature, NMI (53.0 μL, 0.658 mmol) and TCFH (93.3 mg, 0.326 mmol). The resulting mixture was stirred at room temperature for 2 h. A saturated aqueous solution of NaHCO₃ (20 mL) and EtOAc (5 mL) were added to the reaction mixture. The aqueous layer was extracted with EtOAc (3×5 mL). The combined organics were dried over MgSO₄, filtered and concentrated to dryness. The residue was purified by flash chromatography on silica gel (using a gradient of 10 to 100% EtOAc:Heptane as eluent) to afford the title compound as a white solid (57 mg, yield: 71%). Chiral purity: 97%; rt=3.05 min (second eluting enantiomer). For information, first eluting enantiomer rt=2.65 min. Both measured by HPLC (CHIRALPAK IG-u from Daicel, eluant: iPrOH:Heptane 50%—DEA 0.1%). LC-MS m/z [M+H]⁺: 493.1/494.9; purity: 99%. ¹H NMR (400 MHz, DMSO-d₆) δ 10.38 (s, 1H), 8.66 (dd, J=4.8, 1.7 Hz, 1H), 8.29-8.20 (m, 2H), 7.75 (m, 1H), 7.56 (m, 1H), 7.48 (dd, J=8.0, 4.8 Hz, 1H), 7.43 (s, 1H), 4.73-4.57 (m, 2H), 3.81 (q, J=6.6 Hz, 1H), 2.53 (s, 3H), 1.48 (d, J=6.6 Hz, 3H). ¹⁹F NMR (376 MHz, DMSO-d₆) δ−60.51, −123.40 (t, J=9.5 Hz).

### Example #226: Enantiomer (10S) or (10R) of N-(4-chloro-2,5-difluoro-phenyl)-2-[3-chloro-5,10-dimethyl-9-oxo-4,8,12-triazatricyclo[9.4.0.0^{2,7}]pentadeca-1(11),2(7),3,5,12,14-hexaen-8-yl]acetamide

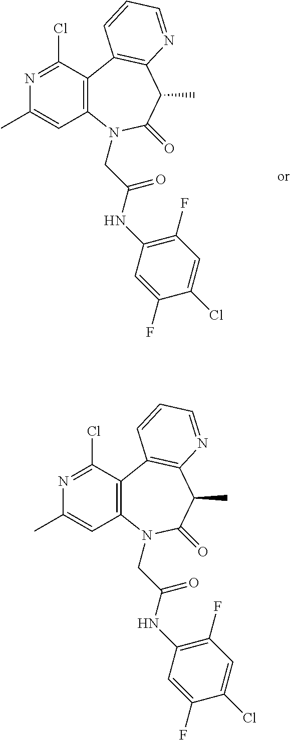 [SMILES: Cc1cc2c(c(Cl)n1)-c1cccnc1[C@H](C)C(=O)N2CC(=O)Nc1cc(F)c(Cl)cc1F ; Cc1cc2c(c(Cl)n1)-c1cccnc1[C@@H](C)C(=O)N2CC(=O)Nc1cc(F)c(Cl)cc1F]

The title product was prepared following the same procedure as for example #225 starting from (10S) or (10R)-2-[3-chloro-5,10-dimethyl-9-oxo-4,8,12-triazatricyclo[9.4.0.0^{2,7}]pentadeca-1(11),2(7),3,5,12,14-hexaen-8-yl]acetic acid hydrochloride NN155a (50.0 mg, 0.136 mmol) and 4-chloro-2,5-difluoroaniline (46.8 mg, 0.272 mmol) to afford the title compound as a white solid (41.2 mg, yield: 60%). LC-MS m/z [M+H]⁺: 477.0/479.0; purity: 96%. ¹H NMR (400 MHz, DMSO-d₆) δ 10.38 (s, 1H), 8.65 (dd, J=4.8, 1.6 Hz, 1H), 8.22 (dd, J=7.9, 1.6 Hz, 1H), 8.10 (dd, J=11.3, 6.9 Hz, 1H), 7.73 (dd, J=10.4, 6.8 Hz, 1H), 7.48 (dd, J=7.9, 4.8 Hz, 1H), 7.42 (s, 1H), 4.71-4.52 (m, 2H), 3.80 (q, J=6.5 Hz, 1H), 2.53 (s, 3H), 1.47 (d, J=6.6 Hz, 3H). ¹⁹F NMR (376 MHz, DMSO-d₆) δ−119.67-−119.81 (m), −128.14-−128.32 (m).

### Example #227: Enantiomer (10S) or (10R) of N-(4-bromo-2-fluoro-phenyl)-2-[3-chloro-5,10-dimethyl-9-oxo-4,8,12-triazatricyclo[9.4.0.0^{2,7}]pentadeca-1(11),2(7),3,5,12,14-hexaen-8-yl]acetamide

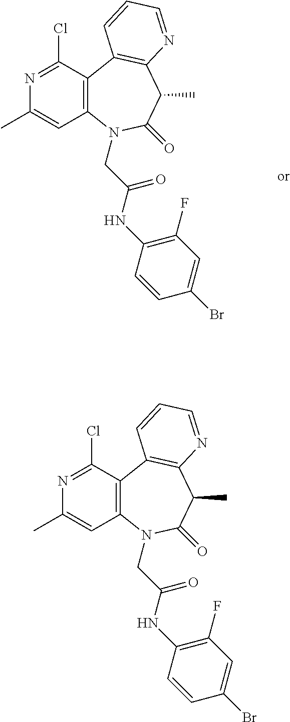 [SMILES: Cc1cc2c(c(Cl)n1)-c1cccnc1[C@H](C)C(=O)N2CC(=O)Nc1ccc(Br)cc1F ; Cc1cc2c(c(Cl)n1)-c1cccnc1[C@@H](C)C(=O)N2CC(=O)Nc1ccc(Br)cc1F]

The title product was prepared following the same procedure as for example #225 starting from (10S) or (10R)-2-[3-chloro-5,10-dimethyl-9-oxo-4,8,12-triazatricyclo[9.4.0.0^{2,7}]pentadeca-1(11),2(7),3,5,12,14-hexaen-8-yl]acetic acid hydrochloride NN155a (25.0 mg, 0.068 mmol) and 4-bromo-2-fluoroaniline (25.8 mg, 0.136 mmol) to afford the title compound as a white solid (30.9 mg, yield: 89%). LC-MS m/z [M+H]⁺: 503.0/505.0/506.9; purity: 98%. ¹H NMR (400 MHz, DMSO-d₆) δ 10.15 (s, 1H), 8.65 (dd, J=4.8, 1.6 Hz, 1H), 8.22 (dd, J=8.0, 1.6 Hz, 1H), 7.91 (t, J=8.6 Hz, 1H), 7.61 (dd, J=10.4, 2.2 Hz, 1H), 7.48 (dd, J=8.0, 4.8 Hz, 1H), 7.42 (s, 1H), 7.39-7.34 (m, 1H), 4.68-4.52 (m, 2H), 3.80 (q, J=6.6 Hz, 1H), 2.52 (s, 3H), 1.47 (d, J=6.6 Hz, 3H). ¹⁹F NMR (376 MHz, DMSO-d₆) δ−122.09 (t, J=9.5 Hz).

### Example #228: Enantiomer (10S) or (10R) of 2-[3-chloro-5,10-dimethyl-9-oxo-4,8,12-triazatricyclo[9.4.0.0^{2,7}]pentadeca-1(15),2,4,6,11,13-hexaen-8-yl]-N-[6-(trifluoromethyl)-3-pyridyl]acetamide

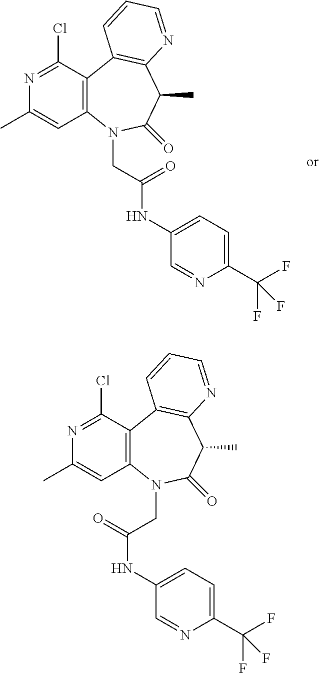 [SMILES: Cc1cc2c(c(Cl)n1)-c1cccnc1[C@@H](C)C(=O)N2CC(=O)Nc1ccc(C(F)(F)F)nc1 ; Cc1cc2c(c(Cl)n1)-c1cccnc1[C@H](C)C(=O)N2CC(=O)Nc1ccc(C(F)(F)F)nc1]

(10S) or (10R)-2-[3-chloro-5,10-dimethyl-9-oxo-4,8,12-triazatricyclo[9.4.0.0^{2,7}]pentadeca-1(11),2(7),3,5,12,14-hexaen-8-yl]acetic acid hydrochloride NN155a (50 mg, 0.1358 mmol, 100 mass %) (50 mg, 0.13 mmol) was dissolved in dry DMF (1.0 mL) and 5-amino-2-(trifluoromethyl)pyridine (29 mg, 0.18 mmol) and HATU (79 mg, 0.17 mmol) were sequentially added. The reaction mixture was stirred at room temperature for 4 h. The reaction mixture was diluted with EtOAc (40 mL) and washed with water (2×30 mL) and brine (50 mL). The organic phase was dried over sodium sulfate, filtered and concentrated under vacuum. The residue was purified by reverse phase HPLC to afford the title compound (15 mg, yield: 23%) as an off-white solid. LC-MS m/z [M+H]⁺: 476.4; purity: 100%. ¹H NMR (400 MHz, DMSO-d₆) δ 8.80 (s, 1H), 8.63 (s, 1H), 8.24 (d, J 22.7 Hz, 1H), 7.86 (d, J 8.7 Hz, 2H), 7.50-7.46 (m, 3H), 7.41 (s, 1H), 4.62-4.51 (m, 2H), 2.54 (s, 3H (under DMSO signal), 1.46 (d, J 6.7 Hz, 3H).

### Example #229: Enantiomer (10S) or (10R) of 2-[3-chloro-5,10-dimethyl-9-oxo-4,8,12-triazatricyclo[9.4.0.0^{2,7}]pentadeca-1(15),2,4,6,11,13-hexaen-8-yl]-N-[5-(trifluoromethyl)-2-pyridyl]acetamide

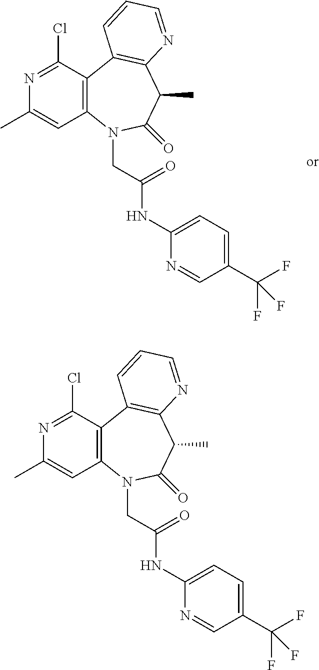 [SMILES: Cc1cc2c(c(Cl)n1)-c1cccnc1[C@@H](C)C(=O)N2CC(=O)Nc1ccc(C(F)(F)F)cn1 ; Cc1cc2c(c(Cl)n1)-c1cccnc1[C@H](C)C(=O)N2CC(=O)Nc1ccc(C(F)(F)F)cn1]

The title product was prepared according to the general procedure 1, starting from (10S) or (10R)-3-chloro-5,10-dimethyl-4,8,12-triazatricyclo[9.4.0.0^{2,7}]pentadeca-1(11),2(7),3,5,12,14-hexaen-9-one N31_A (100 mg, 0.36 mmol) and 2-chloro-N-[5-(trifluoromethyl)-2-pyridyl]acetamide (111.3 mg, 0.46 mmol,). The residue was purified by reverse phase HPLC to afford the title compound (5.4 mg, yield: 4%) as an off-white solid. LC-MS m/z [M+H]⁺: 476.2; purity: 98%.

### Example #230: Enantiomer (10R) or (10S) of 2-[3-chloro-5,10-dimethyl-9-oxo-4,8,12-triazatricyclo[9.4.0.0^{2,7}]pentadeca-1(11),2(7),3,5,12,14-hexaen-8-yl]-N-[6-(difluoromethyl)-3-pyridyl]acetamide

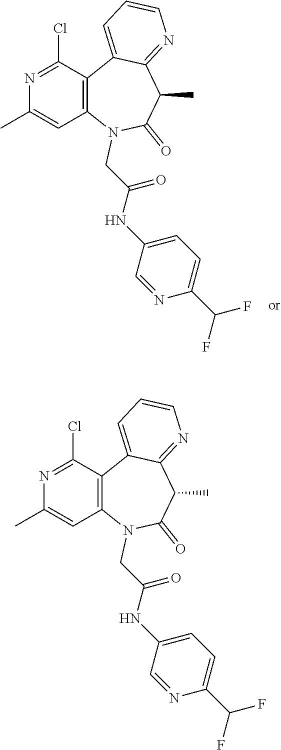 [SMILES: Cc1cc2c(c(Cl)n1)-c1cccnc1[C@@H](C)C(=O)N2CC(=O)Nc1ccc(C(F)F)nc1 ; Cc1cc2c(c(Cl)n1)-c1cccnc1[C@H](C)C(=O)N2CC(=O)Nc1ccc(C(F)F)nc1]

The title product was prepared following the same procedure as for example #128 starting from (10S) or (10R)-2-[3-chloro-5,10-dimethyl-9-oxo-4,8,12-triazatricyclo[9.4.0.0^{2,7}]pentadeca-1(11),2(7),3,5,12,14-hexaen-8-yl]acetic acid hydrochloride NN155a (161 mg, 0.53 mmol) and 6-(difluoromethyl)pyridin-3-amine dihydrochloride (100 mg, 0.44 mmol) using HBTU (1.2 eq.) as the coupling agent in the presence of diisopropylethylamine (2 eq) in DMF at room temperature. After complete conversion, the reaction mixture was diluted with EtOAc (30 mL) and the mixture was washed with HCl 1 M (2×30 mL). The combined acidic aqueous phase from above was basified with sat. NaHCO₃(until pH 7) and extracted with EtOAc (100 mL), the organic layer was dried over MgSO₄, filtered and concentrated under vacuo to afford 87 mg of a brown oil. The crude was purified by reverse phase chromatography (Purification Method P_B) to afford the title compound as a white solid (27 mg, yield: 13%). LC-MS m/z [M+H]⁺: 458.0; purity: 96%. ¹H NMR (400 MHz, DMSO-d₆) δ 10.72 (s, 1H), 8.78 (d, J=2.4 Hz, 1H), 8.66 (dd, J=4.8, 1.6 Hz, 1H), 8.22 (ddd, J=13.3, 8.1, 2.0 Hz, 2H), 7.67 (d, J=8.6 Hz, 1H), 7.49 (dd, J=8.0, 4.8 Hz, 1H), 7.46 (s, 1H), 6.91 (t, 56.1 Hz, 1H), 4.66-4.50 (m, 2H), 3.82 (q, J=6.5 Hz, 1H), 2.53 (s, 3H), 1.48 (d, J=6.5 Hz, 3H).

### Example #233: 2-[(10R)-3-fluoro-5,10-dimethyl-9-oxo-4,8,12-triazatricyclo[9.4.0.0^{2,7}]pentadeca-1(15),2,4,6,11,13-hexaen-8-yl]-N-(4-fluorophenyl)acetamide

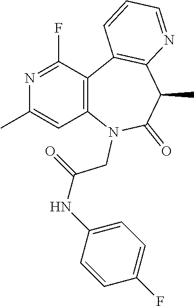

The title compound was prepared according to the procedure described for example #128 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid N62 (20.4 mg, 0.06 mmol) and 4-fluoroaniline (12.2 mg, 0.11 mmol). After complete conversion, the reaction mixture was purified by preparative HPLC (Purification Method P_B) to afford the title compound (16.7 mg, yield: 74%) as a white solid. LC-MS m/z: [M+H]⁺: 409.1; purity: 94%.

### Example #234: N-(4-bromo-3-fluoro-phenyl)-2-[(10R)-3-fluoro-5,10-dimethyl-9-oxo-4,8,12-triazatricyclo[9.4.0.0^{2,7}]pentadeca-1(15),2,4,6,11,13-hexaen-8-yl]acetamide

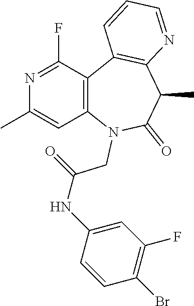

The title compound was prepared according to the procedure described for example #128 starting from 22-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid N62 (17.6 mg, 0.05 mmol) and 4-bromo-3-fluoroaniline (14.7 mg, 0.08 mmol). After complete conversion, the reaction mixture was purified by preparative HPLC (Purification Method P_B) to afford the title compound (13.4 mg, yield: 55%) as a white solid. LC-MS m/z: [M+H]⁺: 487.1; purity: 94%. ¹H NMR (400 MHz, DMSO-d₆) δ 10.58 (s, 1H), 8.67 (d, J=4.7 Hz, 1H), 8.12 (t, J=6.3 Hz, 1H), 7.71 (d, J=11.3 Hz, 1H), 7.63 (t, J=8.6 Hz, 1H), 7.50 (t, J=6.3 Hz, 1H), 7.37 (s, 1H), 7.26 (d, J=8.6 Hz, 1H), 4.56 (s, 2H), 3.78 (q, J=6.5 Hz, 1H), 1.49 (d, J=6.5 Hz, 3H). 3H merged in solvent peak.

### Example #235: N-[4-(difluoromethyl)-3-fluoro-phenyl]-2-[(10R)-3-fluoro-5,10-dimethyl-9-oxo-4,8,12-triazatricyclo[9.4.0.0^{2,7}]pentadeca-1(15),2,4,6,11,13-hexaen-8-yl]acetamide

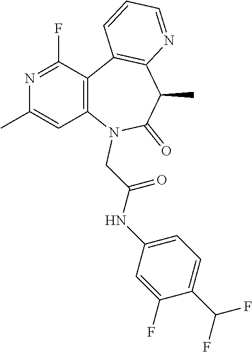 [SMILES: Cc1cc2c(c(F)n1)-c1cccnc1[C@@H](C)C(=O)N2CC(=O)Nc1ccc(C(F)F)c(F)c1]

The title compound was prepared according to the general procedure 1 starting from (3-fluoro-5,10-dimethyl-4,8,12-triazatricyclo[9.4.0.0^{2,7}]pentadeca-1(11),2(7),3,5,12,14-hexaen-9-one N23 (200 mg, 0.78 mmol) and 2-chloro-N-[4-(difluoromethyl)-3-fluoro-phenyl]acetamide NN240 (203 mg, 0.854 mmol). The racemate was separated by chiral chromatography (SFC Whelko Regis Technology, CO₂+Methanol 20%) to give the title compound (244 mg, yield: 68%) as a white solid. ¹H NMR (400 MHz, DMSO-d₆) δ 10.71 (s, 1H), 8.67 (d, J=4.7 Hz, 1H), 8.17-8.09 (m, 1H), 7.69 (d, J=13.0 Hz, 1H), 7.59 (dd, J=8.3, 8.3 Hz, 1H), 7.50 (dd, J=8.0, 4.8 Hz, 1H), 7.40 (d, J=8.6 Hz, 1H), 7.38 (s, 1H), 7.14 (t, J=54.5 Hz, 1H), 4.60 (s, 2H), 3.79 (q, J=6.5 Hz, 1H), 1.49 (d, J=6.6 Hz, 3H), 3H merged in solvent peak. Chiral purity: 99%; rt=3.00 min (first eluting enantiomer). For information, second eluting enantiomer (not recovered) rt=4.22 min. Measured by HPLC, Whelko Regis Technology, Solvent: i-PrOH 50%—Heptane 50%—DEA 0.1%). LC-MS m/z [M+H]⁺: 458.9/460.1; purity: 99.42%

### Example #236: N-[4-(1,1-difluoroethyl)-3-fluoro-phenyl]-2-[(10R)-3-fluoro-5,10-dimethyl-9-oxo-4,8,12-triazatricyclo[9.4.0.0^{2,7}]pentadeca-1(15),2,4,6,11,13-hexaen-8-yl]acetamide

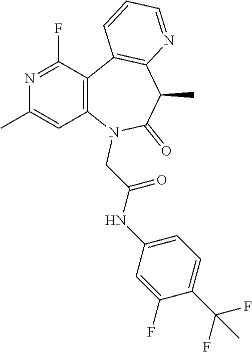 [SMILES: Cc1cc2c(c(F)n1)-c1cccnc1[C@@H](C)C(=O)N2CC(=O)Nc1ccc(C(C)(F)F)c(F)c1]

The title product was prepared according to the general procedure 1, starting from (3-fluoro-5,10-dimethyl-4,8,12-triazatricyclo[9.4.0.0^{2,7}]pentadeca-1(11),2(7),3,5,12,14-hexaen-9-one N23 (129 mg, 0.50 mmol) and 2-chloro-N-(4-(1,1-difluoroethyl)-3-fluorophenyl)acetamide NN241 (133 mg, 0.53 mmol). The residue was purified by flash chromatography on silica gel (using a gradient of 10 to 100% EtOAc in Hexane as eluent). The racemate was separated by chiral chromatography (SFC Chiralpak IA from Daicel, CO₂+Ethanol 30%) to give the title compound (137 mg, yield: 58%) as a white solid. Chiral purity: 99.3%; rt=2.31 min (second eluting enantiomer). For information, first eluting enantiomer rt=1.74 min. Both measured by HPLC, Chiralpak IA from Daicel, Solvent: Ethanol 50%—Heptane 50%—DEA 0.1%). LC-MS m/z [M+H]⁺: 472.9/474.0; purity: 92%.

### Example #237: N-(2,2-difluoroindan-5-yl)-2-[(10R)-3-fluoro-5,10-dimethyl-9-oxo-4,8,12-triazatricyclo[9.4.0.0^{2,7}]pentadeca-1(15),2,4,6,11,13-hexaen-8-yl]acetamide

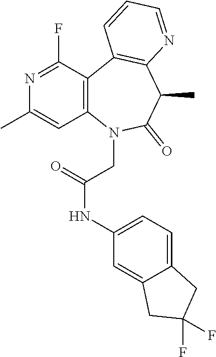 [SMILES: Cc1cc2c(c(F)n1)-c1cccnc1[C@@H](C)C(=O)N2CC(=O)Nc1ccc2c(c1)CC(F)(F)C2]

The title compound was prepared according to the procedure described for example #128 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid N62 (30 mg, 0.058 mmol) and 2,2-difluoro-2,3-dihydro-1H-inden-5-amine NN244 (20 mg, 0.118 mmol) to afford the title compound (12 mg, yield: 44%) as a white solid. LC-MS m/z: [M+H]⁺: 467.5; purity: 98%. ¹H NMR (400 MHz, DMSO-d₆) δ 10.24 (s, 1H), 8.67 (dd, J=4.8, 1.6 Hz, 1H), 8.16-8.09 (m, 1H), 7.60 (s, 1H), 7.50 (dd, J=7.9, 4.7 Hz, 1H), 7.37 (s, 1H), 7.32 (dd, J=8.2, 2.0 Hz, 1H), 7.20 (d, J=8.2 Hz, 1H), 4.61-4.50 (m, 2H), 3.77 (q, J=6.6 Hz, 1H), 3.41 (q, J=15.3 Hz, 4H), 1.49 (d, J=6.6 Hz, 3H). CH₃ protons under DMSO signal. ¹⁹F NMR (376 MHz, DMSO-d₆) δ−71.01 (d, J=4.7 Hz), −92.94 (p, J=14.8 Hz).

### Example #238: N-(2,2-difluoro-1,3-benzodioxol-5-yl)-2-[(10R)-3-fluoro-5,10-dimethyl-9-oxo-4,8,12-triazatricyclo[9.4.0.0^{2,7}]pentadeca-1(15),2,4,6,11,13-hexaen-8-yl]acetamide

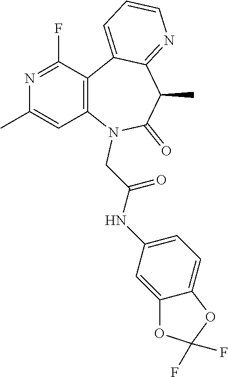 [SMILES: Cc1cc2c(c(F)n1)-c1cccnc1[C@@H](C)C(=O)N2CC(=O)Nc1ccc2c(c1)OC(F)(F)O2]

The title compound was prepared according to the procedure described for example #230 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid N62 (121 mg, 0.28 mmol) and 2,2-difluoro-5-aminobenzodioxole (50 mg, 0, 28 mmol). The crude was purified by trituration in EtOAc, the solid was filtered off and dried under vacuum to afford the title compound as a white solid (31 mg, yield: 20%). LC-MS m/z [M+H]⁺: 471.1; purity: 97%. ¹H NMR (400 MHz, DMSO-d₆) δ 10.50 (s, 1H), 8.68 (dd, J=4.8, 1.6 Hz, 1H), 8.13 (ddd, J=8.1, 4.6, 1.6 Hz, 1H), 7.74 (d, J=2.0 Hz, 1H), 7.50 (dd, J=7.9, 4.8 Hz, 1H), 7.37 (d, J=7.2 Hz, 2H), 7.25 (dd, J=8.8, 2.0 Hz, 1H), 4.56 (d, J=1.9 Hz, 2H), 3.79 (q, J=6.5 Hz, 1H), hidden methyl under DMSO peak, 1.49 (d, J=6.6 Hz, 3H).

### Example #239: 2-[(10R)-3-fluoro-5,10-dimethyl-9-oxo-4,8,12-triazatricyclo[9.4.0.0^{2,7}]pentadeca-1(15),2,4,6,11,13-hexaen-8-yl]-N-(3-fluoro-6-isoquinolyl)acetamide

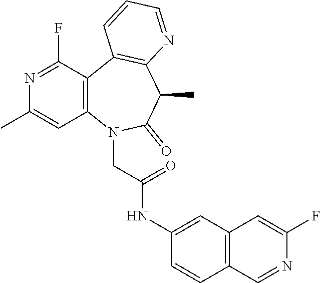 [SMILES: Cc1cc2c(c(F)n1)-c1cccnc1[C@@H](C)C(=O)N2CC(=O)Nc1ccc2cnc(F)cc2c1]

### Step 1: Synthesis of 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetamide #239_1

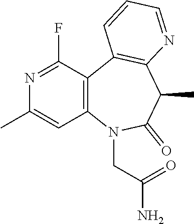

To a solution of (10R)-3-fluoro-5,10-dimethyl-4,8,12-triazatricyclo[9.4.0.0^{2,7}]pentadeca-1(11),2(7),3,5,12,14-hexaen-9-one N61 (1.00 g, 3.89 mmol) in DMF (20 mL) were added K₂CO₃ (650 mg, 4.66 mmol) and Iodoacetamide (800 mg, 4.28 mmol). The reaction mixture was stirred at room temperature for 6 h. After complete conversion, water (20 mL) was added and the reaction mixture was extracted with EtOAc (3×40 mL). The combined organic extracts were washed with brine (50 mL), dried over Na₂SO₄, filtered and concentrated under vacuum. The residue was purified by flash chromatography on silica gel (using a gradient of 0 to 20% MeOH in DCM as eluent) to afford the title compound (738 mg, yield: 60%) as a white solid. LC-MS m/z: [M+H]⁺: 315.0; purity: 99%. ¹H NMR (400 MHz, DMSO-d₆) δ 8.66 (dd, J=4.8, 1.6 Hz, 1H), 8.13-8.05 (m, 1H), 7.58 (s, 1H), 7.47 (dd, J=7.9, 4.8 Hz, 1H), 7.26 (s, 1H), 7.14 (s, 1H), 4.43-4.15 (m, 2H), 3.73 (q, J=6.6 Hz, 1H), 1.48 (d, J=6.6 Hz, 3H). CH₃ protons under DMSO signal. Chiral purity: 99%; rt=3.03 min (second eluting enantiomer). For information, first eluting enantiomer rt=1.87 min. Both measured by HPLC (Chiralpak AD from Daicel, EtOH 50%—heptane 50%—DEA 0.1%).

### Step 2: Synthesis of 2-[(10R)-3-fluoro-5,10-dimethyl-9-oxo-4,8,12-triazatricyclo[9.4.0.0^{2,7}]pentadeca-1(15),2,4,6,11,13-hexaen-8-yl]-N-(3-fluoro-6-isoquinolyl)acetamide #239

Under N₂ atmosphere, to a solution of 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetamide #239_1 (50 mg, 0.16 mmol) and 6-bromo-3-fluoroisoquinoline (45 mg, 0.19 mmol) in dry 1,4-Dioxane (1.6 mL) were added Cs₂CO₃ (156 mg, 0.48 mmol), BrettPhos Pd G3 (15 mg, 0.02 mmol) and BrettPhos (9 mg, 0.02 mmol). The reaction mixture was sonicated a few seconds then heated at 100° C. for 3 h. After complete conversion, water (40 mL) was added and the reaction mixture was extracted with DCM (3×40 mL). The combined organic extracts were dried over MgSO₄, filtered, and concentrated under vacuum. The residue was purified by preparative HPLC (Purification Method P_B) and then by Chiral SFC (Reprosil Chiral NR-R from Dr Maisch, CO₂+EtOH 30%) to afford the title compound (16.4 mg, yield: 22%) as a white solid. LC-MS m/z: [M+H]⁺: 460.0; purity: 99%. ¹H NMR (400 MHz, DMSO-d₆) δ 10.75 (s, 1H), 8.96 (s, 1H), 8.68 (dd, J=4.8, 1.7 Hz, 1H), 8.40 (d, J=2.0 Hz, 1H), 8.17-8.10 (m, 2H), 7.60 (dd, J=8.9, 2.0 Hz, 1H), 7.54-7.49 (m, 2H), 7.40 (s, 1H), 4.74-4.58 (m, 2H), 3.80 (q, J=6.6 Hz, 1H), 1.50 (d, J=6.6 Hz, 3H). CH₃ protons under DMSO signal. ¹⁹F NMR (376 MHz, DMSO-d₆) δ−70.94 (d, J=5.3 Hz), −78.06-−78.11 (m). Chiral purity: 99%; rt=3.31 min (first eluting enantiomer). For information, second eluting enantiomer rt=4.60 min. Both measured by HPLC (Reprosil Chiral NR-R from Dr Maisch, EtOH 50%—heptane 50%—DEA 0.1%).

### Example #240: N-(1-chloro-6-isoquinolyl)-2-[(10R)-3-fluoro-5,10-dimethyl-9-oxo-4,8,12-triazatricyclo[9.4.0.0^{2,7}]pentadeca-1(15),2,4,6,11,13-hexaen-8-yl]acetamide

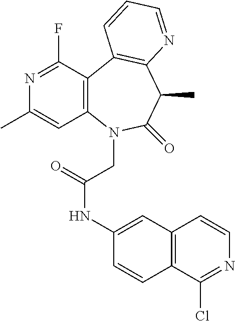 [SMILES: Cc1cc2c(c(F)n1)-c1cccnc1[C@@H](C)C(=O)N2CC(=O)Nc1ccc2c(Cl)nccc2c1]

The title compound was prepared according to the same procedure as for example #239 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetamide #239_1 (50 mg, 0.16 mmol), 6-bromo-1-chloroisoquinoline (48 mg, 0.19 mmol), XantPhos Pd G3 (15 mg, 0.02 mmol) and XantPhos (9.5 mg, 0.02 mmol). The residue was purified by preparative HPLC (Purification Method P_A by Purification Method P_B) and then by Chiral SFC (Reprosil Chiral NR-R from Dr Maisch, CO₂+MeOH 40%) to afford the title compound (11.6 mg, yield: 15%) as a white solid. LC-MS (Method A2) m/z: [M+H]⁺: 476.0; rt: 4.38 min; purity: 99%. LC-MS m/z: [M+H]⁺: 476.0; purity: 99%. ¹H NMR (400 MHz, DMSO-d₆) δ 10.87 (s, 1H), 8.68 (dd, J=4.8, 1.7 Hz, 1H), 8.46 (d, J=2.1 Hz, 1H), 8.27-8.19 (m, 2H), 8.14 (ddd, J=7.9, 4.6, 1.7 Hz, 1H), 7.86-7.73 (m, 2H), 7.51 (dd, J=7.9, 4.8 Hz, 1H), 7.41 (s, 1H), 4.75-4.58 (m, 2H), 3.80 (q, J=6.6 Hz, 1H), 1.50 (d, J=6.6 Hz, 3H). CH₃ protons under DMSO signal. ¹⁹F NMR (376 MHz, DMSO-d₆) δ−70.92 (d, J=4.6 Hz). Chiral purity: 100%; rt=3.90 min (second eluting enantiomer). For information, first eluting enantiomer rt=2.27 min. Both measured by HPLC (Chiralpak IB from Daicel, MeOH 100%—DEA 0.1%).

### Example #241: N-(3-chloro-4-fluoro-phenyl)-2-[(10R)-3-fluoro-5,10-dimethyl-9-oxo-4,8,12-triazatricyclo[9.4.0.0^{2,7}]pentadeca-1(15),2,4,6,11,13-hexaen-8-yl]acetamide

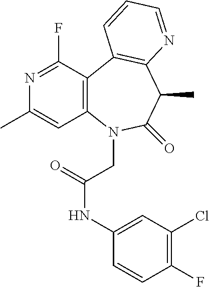 [SMILES: Cc1cc2c(c(F)n1)-c1cccnc1[C@@H](C)C(=O)N2CC(=O)Nc1ccc(F)c(Cl)c1]

The title compound was prepared according to the procedure described for example #128 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid (trifluoroacetic acid salt) N62 (60 mg, 0.116 mmol) and 3-chloro-4-fluoroaniline (34 mg, 0.234 mmol). Purification by preparative HPLC (Purification Method P_B) afforded the title compound (32 mg, yield: 62%) as a white solid. LC-MS m/z: [M+H]⁺: 443.1/445.0; purity: 98%. ¹H NMR (400 MHz, DMSO-d₆) δ 10.48 (s, 1H), 8.67 (dd, J=4.8, 1.7 Hz, 1H), 8.13 (ddd, J=8.0, 4.6, 1.7 Hz, 1H), 7.91 (dd, J=6.8, 2.5 Hz, 1H), 7.50 (dd, J=8.0, 4.8 Hz, 1H), 7.45-7.40 (m, 1H), 7.38 (t, J=9.0 Hz, 1H), 7.37 (s, 1H), 4.55 (s, 2H), 3.78 (q, J=6.6 Hz, 1H), 1.49 (d, J=6.6 Hz, 3H). CH₃ protons under DMSO signal. ¹⁹F NMR (376 MHz, DMSO-d₆) δ−70.97 (d, J=4.6 Hz), −122.62-−122.77 (m).

### Example #242: N-(4-chloro-2-fluoro-phenyl)-2-[(10R)-3-fluoro-5,10-dimethyl-9-oxo-4,8,12-triazatricyclo[9.4.0.0^{2,7}]pentadeca-1(15),2,4,6,11,13-hexaen-8-yl]acetamide

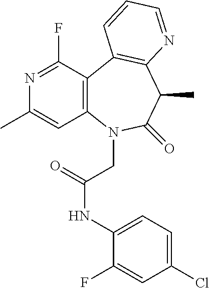 [SMILES: Cc1cc2c(c(F)n1)-c1cccnc1[C@@H](C)C(=O)N2CC(=O)Nc1ccc(Cl)cc1F]

The title compound was prepared according to the procedure described for example #128 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid (trifluoroacetic acid salt) N62 (60 mg, 0.116 mmol) and 4-chloro-2-fluoroaniline (26 μL, 0.230 mmol). Purification by preparative HPLC (Purification Method P_B) afforded the title compound (11 mg, yield: 21%) as a white solid. LC-MS m/z: [M+H]⁺: 443.1/444.9; purity: 96%. ¹H NMR (400 MHz, DMSO-d₆) δ 10.18 (s, 1H), 8.67 (dd, J=4.8, 1.7 Hz, 1H), 8.15-8.07 (m, 1H), 7.97 (t, J=8.7 Hz, 1H), 7.55-7.45 (m, 2H), 7.35 (s, 1H), 7.25 (dd, J=8.7, 2.0 Hz, 1H), 4.74-4.53 (m, 2H), 3.77 (q, J=6.6 Hz, 1H), 1.49 (d, J=6.6 Hz, 3H). CH₃ protons under DMSO signal. ¹⁹F NMR (376 MHz, DMSO-d₆) δ−70.97 (d, J=4.7 Hz), −122.07 (t, J=8.7 Hz).

### Example #243: N-(5-chloro-3-thienyl)-2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetamide

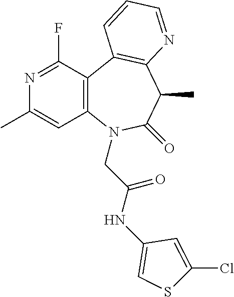 [SMILES: Cc1cc2c(c(F)n1)-c1cccnc1[C@@H](C)C(=O)N2CC(=O)Nc1csc(Cl)c1]

The title compound was prepared according to the procedure described for example #128 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid (trifluoroacetic acid salt N62) (60.0 mg, 0.116 mmol) and 5-chlorothiophen-3-amine hydrochloride (39.5 mg, 0.232 mmol) The reaction mixture was directly purified by preparative HPLC (Purification Method P_B) to afford the title compound as a white-grey solid (38.6 mg, yield: 76%). LC-MS m/z [M+H]⁺: 431.0/432.8; purity: 95%. ¹H NMR (400 MHz, DMSO-d₆) δ 10.66 (s, 1H), 8.67 (dd, J=4.8, 1.7 Hz, 1H), 8.15-8.08 (m, 1H), 7.49 (dd, J=7.9, 4.8 Hz, 1H), 7.35 (s, 1H), 7.33 (d, J=1.8 Hz, 1H), 7.07 (d, J=1.8 Hz, 1H), 4.51 (s, 2H), 3.77 (q, J=6.6 Hz, 1H), 1.48 (d, J=6.6 Hz, 3H) (+one CH₃ present under the signal of the DMSO-d₆). ¹⁹F NMR (376 MHz, DMSO-d₆) δ−70.97 (d, J=5.3 Hz).

### Example #244: 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]-N-(3-fluoro-4-isopropyl-phenyl)acetamide

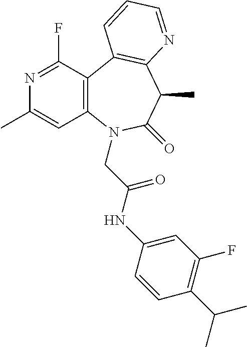 [SMILES: Cc1cc2c(c(F)n1)-c1cccnc1[C@@H](C)C(=O)N2CC(=O)Nc1ccc(C(C)C)c(F)c1]

The title compound was prepared according to the procedure described for example #128 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid (trifluoroacetic acid salt) N62 (60.0 mg, 0.116 mmol) and 3-fluoro-4-(propan-2-yl)aniline (35.5 mg, 0.232 mmol). The reaction mixture was directly purified by preparative HPLC (Purification Method P_A followed by Purification Method P_B) to afford the title compound as a white-grey solid (31.6 mg, yield: 59%). LC-MS m/z [M+H]⁺: 451.3; purity: 98%. ¹H NMR (400 MHz, DMSO-d₆) δ 10.36 (s, 1H), 8.67 (dd, J=4.8, 1.6 Hz, 1H), 8.16-8.09 (m, 1H), 7.53-7.44 (m, 2H), 7.36 (s, 1H), 7.30-7.18 (m, 2H), 4.61-4.48 (m, 2H), 3.78 (q, J=6.6 Hz, 1H), 3.10 (p, J=6.9 Hz, 1H), 1.49 (d, J=6.6 Hz, 3H), 1.19 (d, J=6.9 Hz, 6H) (+one CH₃ present under the signal of the DMSO-d₆). ¹⁹F NMR (376 MHz, DMSO-d₆) δ−70.99 (d, J=5.1 Hz), −118.25 (dd, J=13.3, 8.3 Hz).

### Example #245: N-(4-chloro-2,5-difluoro-phenyl)-2-[(10R)-3-fluoro-5,10-dimethyl-9-oxo-4,8,12-triazatricyclo[9.4.0.0^{2,7}]pentadeca-1(15),2,4,6,11,13-hexaen-8-yl]acetamide

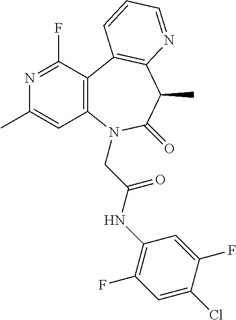 [SMILES: Cc1cc2c(c(F)n1)-c1cccnc1[C@@H](C)C(=O)N2CC(=O)Nc1cc(F)c(Cl)cc1F]

The title compound was prepared according to the procedure described for example #128 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid (trifluoroacetic acid salt) N62 (45 mg, 0.14 mmol) and 4-chloro-2,5-difluoro-aniline. The crude was purified by column chromatography and by reverse phase HPLC (acidic elution) to yield the title compound as an off white solid (1 mg, yield: 1.5%). LC-MS m/z [M+H]⁺: 461.2, 463.2; purity: 100%. ¹H NMR (400 MHz, DMSO-d₆) δ 10.39 (s, 1H), 8.67 (dd, J=4.8, 1.7 Hz, 1H), 8.16-8.05 (m, 2H), 7.72 (dd, J=10.5, 6.8 Hz, 1H), 7.49 (dd, J=7.9, 4.8 Hz, 1H), 7.34 (s, 1H), 4.73 (d, J=17.2 Hz, 1H), 4.56 (d, J=17.1 Hz, 1H), 3.77 (q, J=6.7 Hz, 1H), 1.49 (d, J=6.6 Hz, 3H), three protons signal merged with DMSO solvent residual signal.

### Example #246: N-[4-(1-fluorocyclopropyl)phenyl]-2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetamide

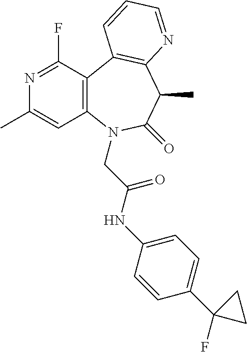 [SMILES: Cc1cc2c(c(F)n1)-c1cccnc1[C@@H](C)C(=O)N2CC(=O)Nc1ccc(C2(F)CC2)cc1]

The title compound was prepared according to the procedure described for example #128 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid (trifluoroacetic acid salt) N62 (117.0 mg, 0.232 mmol) and 4-(1-fluorocyclopropyl)aniline hydrochloride NN141 (CAS: 1785095-13-7, 38.8 mg, 0.203 mmol) The residue was purified by flash chromatography on silica gel (using a gradient of 20 to 100% EtOAc:Heptane as eluent) followed by preparative HPLC (Purification Method P_B) to afford the title compound as a yellowish solid (58.0 mg, yield: 63%). LC-MS m/z [M+H]⁺: 449.1; purity: 97%. ¹H NMR (400 MHz, DMSO-d₆) δ 10.31 (s, 1H), 8.67 (dd, J=4.8, 1.7 Hz, 1H), 8.13 (ddd, J=7.9, 4.9, 1.7 Hz, 1H), 7.58 (d, J=8.5 Hz, 2H), 7.50 (dd, J=7.9, 4.8 Hz, 1H), 7.37 (s, 1H), 7.25 (d, J=8.5 Hz, 2H), 4.62-4.49 (m, 2H), 3.78 (q, J=6.5 Hz, 1H), 1.49 (d, J=6.5 Hz, 3H), 1.47-1.36 (m, 2H), 1.12-1.04 (m, 2H). (+one CH₃ present under the signal of the DMSO-d₆). ¹⁹F NMR (376 MHz, DMSO-d₆) δ−71.01 (d, J=4.9 Hz), −175.89 (p, J=9.5 Hz).

### Example #247: 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl)-N-[2-fluoro-4-(trifluoromethyl)phenyl]acetamide

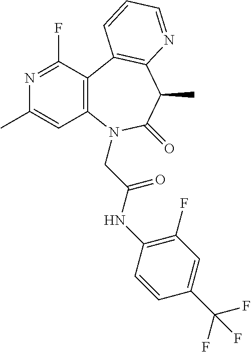 [SMILES: Cc1cc2c(c(F)n1)-c1cccnc1[C@@H](C)C(=O)N2CC(=O)Nc1ccc(C(F)(F)F)cc1F]

The title compound was prepared according to the procedure described for example #225 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid (trifluoroacetic acid salt) N62 (50.0 mg, 0.099 mmol) and 4-amino-3-fluorobenzotrifluoride (37.3 mg, 0.198 mmol) in MeCN (1.0 mL). The residue was purified by flash chromatography on silica gel (using a gradient of 20 to 80% EtOAc:Heptane as eluent) to afford the title compound as a yellowish solid (20.6 mg, yield: 44%). LC-MS m/z [M+H]⁺: 477.2; purity: 96%. ¹H NMR (400 MHz, DMSO-d₆) δ 10.42 (bs., 1H), 8.67 (dd, J=4.7, 1.7 Hz, 1H), 8.27 (, J=8.9 Hz, 1H), 8.12 (ddd, J=7.9, 4.7, 1.7 Hz, 1H), 7.76 (dd, J=11.2, 1.8 Hz, 1H), 7.56 (dd, J=8.9, 1.8 Hz, 1H), 7.49 (dd, J=7.9, 4.7 Hz, 1H), 7.36 (s, 1H), 4.76 (d, J=17.2 Hz, 1H), 4.79-4.67 (m, 1H), 3.79 (q, J=6.6 Hz, 1H), 1.49 (d, J=6.6 Hz, 3H) (+one CH₃ present under the signal of the DMSO-d₆). ¹⁹F NMR (376 MHz, DMSO-d₆) δ−60.50, −70.95 (d, J=4.7 Hz), −123.40 (m).

### Example #248: 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]-N-[3-fluoro-4-(1-fluorocyclopropyl)phenyl]acetamide

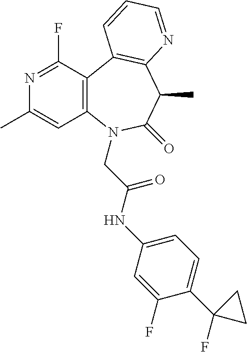 [SMILES: Cc1cc2c(c(F)n1)-c1cccnc1[C@@H](C)C(=O)N2CC(=O)Nc1ccc(C2(F)CC2)c(F)c1]

The title compound was prepared according to the procedure described for example #128 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid (trifluoroacetic acid salt) N62 (38.0 mg, 0.075 mmol) and 3-fluoro-4-(1-fluorocyclopropyl)aniline hydrochloride (15.0 mg, 0.069 mmol). The residue was purified by preparative HPLC (Purification Method P_B) to afford the title compound as a white solid (12.8 mg, yield: 39%). LC-MS m/z [M+H]⁺: 467.2; purity: 98%. ¹H NMR (400 MHz, DMSO-d₆) δ 10.58 (s, 1H), 8.67 (dd, J=4.8, 1.7 Hz, 1H), 8.13 (ddd, J=8.0, 4.7, 1.7 Hz, 1H), 7.59 (dd, J=13.1, 2.0 Hz, 1H), 7.53-7.44 (m, 2H), 7.36 (s, 1H), 7.30 (dd, J=8.6, 2.0 Hz, 1H), 4.57 (s, 2H), 3.78 (q, J=6.6 Hz, 1H), 1.49 (d, J=6.6 Hz, 3H), 1.36 (dd, J=18.3, 6.9 Hz, 2H), 1.05 (td, J=8.2, 6.3 Hz, 2H). (+one CH₃ present under the signal of the DMSO-d₆). ¹⁹F NMR (376 MHz, DMSO-d₆) δ−70.98 (d, J=4.7 Hz), −114.42 (ddd, J=13.1, 8.6, 4.7 Hz), −164.89-−165.09 (m).

### Example #249: N-(4-chloro-2,3-difluoro-phenyl)-2-[(10R)-3-fluoro-5,10-dimethyl-9-oxo-4,8,12-triazatricyclo[9.4.0.0^{2,7}]pentadeca-1(15),2,4,6,11,13-hexaen-8-yl]acetamide

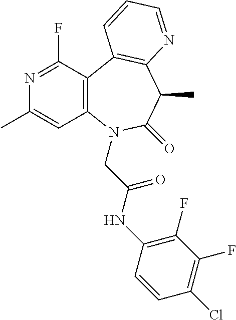 [SMILES: Cc1cc2c(c(F)n1)-c1cccnc1[C@@H](C)C(=O)N2CC(=O)Nc1ccc(Cl)c(F)c1F]

The title compound was prepared according to the procedure described for example #128 starting from 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid (trifluoroacetic acid salt) N62 (45 mg, 0.14 mmol) and 4-chloro-2,3-difluoro-aniline. The crude was purified by column chromatography and by reverse phase HPLC (acidic elution) to yield the title compound as an off white solid (2.3 mg, yield: 3%). LC-MS m/z [M+H]⁺: 461.1, 463.1; purity: 96%. ¹H NMR (400 MHz, CD30D) δ 8.66 (dd, J=4.8, 1.7 Hz, 1H), 8.17 (ddd, J=7.9, 4.6, 1.7 Hz, 1H), 7.88-7.78 (m, 1H), 7.50 (dd, J=8.0, 4.9 Hz, 1H), 7.38 (s, 1H), 7.34-7.23 (m, 1H), 4.78 (d, J=16.9 Hz, 1H), 4.58 (d, J=16.8 Hz, 1H), 3.81 (q, J=6.7 Hz, 1H), 2.59 (s, 3H), 1.67 (d, J=6.6 Hz, 3H). One exchangeable proton not observed,

### Example #250: Enantiomer (10S) or (10R) of N-[4-(difluoromethyl)phenyl]-2-[5,10-dimethyl-9-oxo-4,8,12-triazatricyclo[9.4.0.0²,l]pentadeca-1(15),2,4,6,11,13-hexaen-8-yl]acetamide

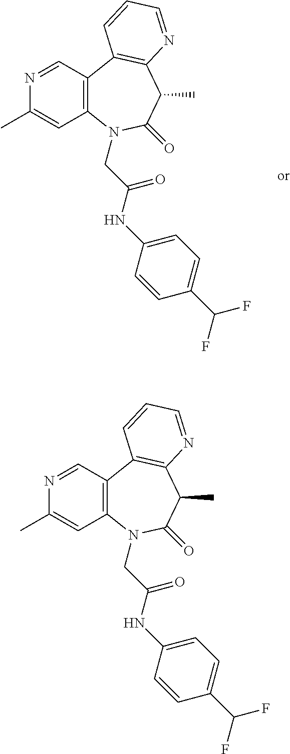 [SMILES: Cc1cc2c(cn1)-c1cccnc1[C@H](C)C(=O)N2CC(=O)Nc1ccc(C(F)F)cc1 ; Cc1cc2c(cn1)-c1cccnc1[C@@H](C)C(=O)N2CC(=O)Nc1ccc(C(F)F)cc1]

The title compound was prepared according to the general procedure 1 starting from 5,10-dimethyl-4,8,12-triazatricyclo[9.4.0.0^{2,7}]pentadeca-1(11),2(7),3,5,12,14-hexaen-9-one N123 (320 mg, 1.34 mmol) and 2-chloro-N-[4-(difluoromethyl)phenyl]acetamide (CAS 872533-93-2, 350 mg, 1.59 mmol). The racemate was separated by Chiral SFC (Chiralpak IG from Daicel, CO₂+EtOH 35%) to afford the title compound (163.5 mg, yield: 29%) as a white solid. LC-MS m/z: [M+H]⁺: 423; purity: 99%. ¹H NMR (400 MHz, DMSO-d₆) δ 10.46 (s, 1H), 8.73 (s, 1H), 8.67 (dd, J=4.8, 1.7 Hz, 1H), 8.15 (dd, J=7.8, 1.7 Hz, 1H), 7.70 (d, J=8.4 Hz, 2H), 7.56-7.46 (m, 3H), 7.38 (s, 1H), 6.97 (t, J=56.1 Hz, 1H), 4.67-4.52 (m, 2H), 3.65 (q, J=6.7 Hz, 1H), 2.56 (s, 3H), 1.50 (d, J=6.7 Hz, 3H). Chiral purity: 100%; rt=4.23 min (second eluting enantiomer). For information, first eluting enantiomer rt=2.67 min. Both measured by HPLC (Chiralpak IG-u from Daicel, EtOH 50%—heptane 50%—DEA 0.1%).

### Example #251: Enantiomer (10S) or (10R) of N-(4-chloro-3-fluoro-phenyl)-2-[5,10-dimethyl-9-oxo-4,8,12-triazatricyclo[9.4.0.0^{2,7}]pentadeca-1(15),2,4,6,11,13-hexaen-8-yl]acetamide

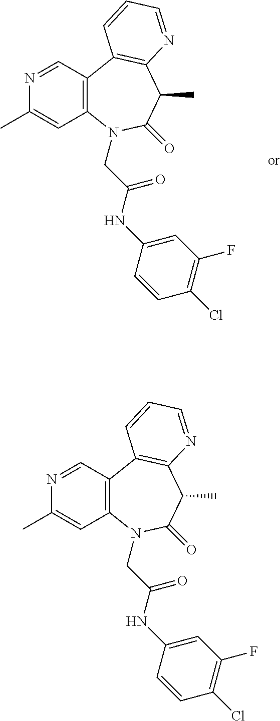 [SMILES: Cc1cc2c(cn1)-c1cccnc1[C@@H](C)C(=O)N2CC(=O)Nc1ccc(Cl)c(F)c1 ; Cc1cc2c(cn1)-c1cccnc1[C@H](C)C(=O)N2CC(=O)Nc1ccc(Cl)c(F)c1]

The title product was prepared according to the general procedure 1, starting from intermediate N123 (5.0 g, 20.9 mmol) and 2-chloro-N-(4-chloro-3-fluoro-phenyl)acetamide (5.1 g, 23.0 mmol). The racemate was separated by SFC (column Chiralpak IG from Daicel 40% Ethanol+60% CO₂ at 40° C.) to yield the pure enantiomer as a white solid (2.28 g, yield: 25%). LC-MS (Method B3) m/z [M+H]⁺: 425.0; rt: 3.99 min; purity>99%. LC-MS m/z [M+H]⁺: 425.0; purity>99%. ¹H NMR (400 MHz, DMSO-d₆) δ 10.60 (s, 1H), 8.73 (s, 1H), 8.67 (s, 1H), 8.16 (d, J=7.9 Hz, 1H), 7.75 (d, J=12.2 Hz, 1H), 7.57-7.47 (m, 2H), 7.37 (s, 1H), 7.32 (d, J=8.7 Hz, 1H), 4.61 (d, J=16.6 Hz, 1H), 4.53 (d, J=16.6 Hz, 1H), 3.74-3.56 (q, J=6.5 Hz, 1H), 2.56 (s, 3H), 1.49 (d, J=6.5 Hz, 3H). Chiral purity>99%; rt=2.89 min (second eluting enantiomer). For information, first eluting enantiomer rt=1.97 min. Both measured by HPLC, Chiralpak IG-u from Daicel, EtOH 50%—heptane 50%—DEA 0.1%)

### Example #252: Enantiomer (10S) or (10R) of N-[4-(difluoromethyl)-3-fluoro-phenyl]-2-[5,10-dimethyl-9-oxo-4,8,12-triazatricyclo[9.4.0.0^{2,7}]pentadeca-1(15),2,4,6,11,13-hexaen-8-yl]acetamide

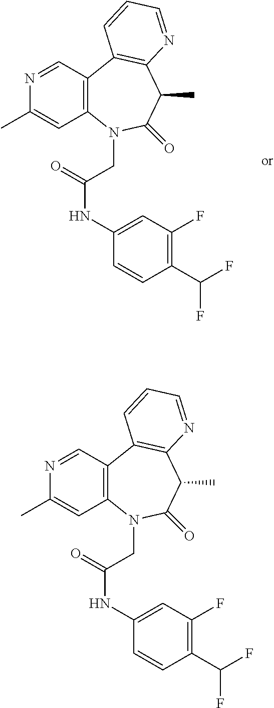 [SMILES: Cc1cc2c(cn1)-c1cccnc1[C@@H](C)C(=O)N2CC(=O)Nc1ccc(C(F)F)c(F)c1 ; Cc1cc2c(cn1)-c1cccnc1[C@H](C)C(=O)N2CC(=O)Nc1ccc(C(F)F)c(F)c1]

The title product was prepared according to the general procedure 1, starting from intermediate N123 (240 mg, 1.003 mmol) and 2-chloro-N-[4-(difluoromethyl)-3-fluoro-phenyl]acetamide NN240 (262 mg, 1.10 mmol) The racemate was separated by chiral chromatography (SFC Chiralpak IG from Daicel, CO₂+Ethanol 20%) to give the title compound (167 mg, yield: 38%) as a solid. Chiral purity: 93.6%; rt=1.81 min (second eluting enantiomer). For information, first eluting enantiomer rt=2.15 min. Both measured by HPLC, Chiralpak IG from Daicel, Solvent: i-PrOH 50%—Heptane 50%—DEA 0.1%). LC-MS m/z [M+H]⁺: 441.2; purity: 98.11%. ¹H NMR (400 MHz, DMSO-d₆) δ 10.71 (s, 1H), 8.73 (s, 1H), 8.68 (d, J=4.8 Hz, 1H), 8.16 (d, J=1.9 Hz, 1H), 7.69 (d, J=13.0 Hz, 1H), 7.59 (t, J=8.3 Hz, 1H), 7.55-7.49 (m, 1H), 7.41 (d, J=8.5 Hz, 1H), 7.38 (s, 1H), 7.14 (t, J=54.6 Hz, 1H), 4.60 (q, J=16.9 Hz, 2H), 3.65 (q, J=6.5 Hz, 1H), 2.56 (s, 3H), 1.50 (d, J=6.6 Hz, 3H).

### Example #253: Enantiomer (10R) or (10S) of N-(3-chloro-4-fluoro-phenyl)-2-(5,10-dimethyl-9-oxo-4,8,12-triazatricyclo[9.4.0.0^{2,7}]pentadeca-1(11),2(7).3,5,12,14-hexaen-8-yl)acetamide

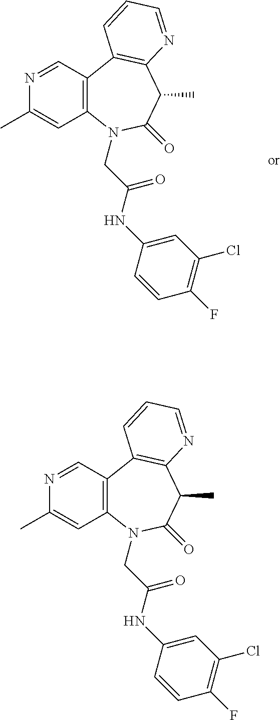 [SMILES: Cc1cc2c(cn1)-c1cccnc1[C@H](C)C(=O)N2CC(=O)Nc1ccc(F)c(Cl)c1 ; Cc1cc2c(cn1)-c1cccnc1[C@@H](C)C(=O)N2CC(=O)Nc1ccc(F)c(Cl)c1]

The title product was prepared according to the general procedure 1, starting from intermediate N123 (80.0 mg, 0.334 mmol) and 2-chloro-N-(3-chloro-4-fluoro-phenyl)acetamide (CAS: 96980-64-2, 89.0 mg, 0.401 mmol). The racemate was separated by chiral chromatography (SFC CHIRALPAK IA from Daicel, eluant: CO₂+ethanol 20%) to afford the title compound as a white-orange solid (39.8 mg, yield: 28%). Chiral purity: 100%; rt=1.87 min (second eluting enantiomer). For information, first eluting enantiomer rt=1.59 min. Both measured by HPLC (CHIRALPAK IA from Daicel, eluant: EtOH:Heptane 50%—DEA 0.1%). LC-MS m/z [M+H]⁺: 425.0/427.0; purity: ¹H NMR (400 MHz, DMSO-d₆) δ 10.48 (s, 1H), 8.73 (s, 1H), 8.67 (dd, J=4.8, 1.6 Hz, 1H), 8.16 (dd, J=7.8, 1.6 Hz, 1H), 7.92 (dd, J=6.9, 2.5 Hz, 1H), 7.52 (dd, J=7.8, 4.8 Hz, 1H), 7.46-7.41 (m, 1H), 7.41-7.34 (m, 2H), 4.63-4.47 (m, 2H), 3.64 (q, J=6.6 Hz, 1H), 2.56 (s, 3H), 1.49 (d, J=6.6 Hz, 3H). ¹⁹F NMR (376 MHz, DMSO-d₆) δ−122.69-−122.78 (m).

### Example #254: N-(4-chloro-3-fluoro-phenyl)-2-(4,5,10-trimethyl-3,9-dioxo-4,8,12-triazatricyclo[9.4.0.0^{2,7}]pentadeca-1(15),2(7),5,11,13-pentaen-8-yl)acetamide

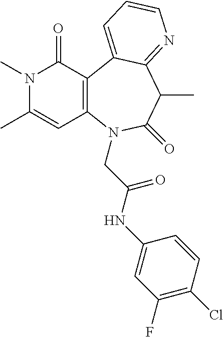 [SMILES: Cc1cc2c(c(=O)n1C)-c1cccnc1C(C)C(=O)N2CC(=O)Nc1ccc(Cl)c(F)c1]

The title compound was prepared according to the procedure described for example #174 starting from Intermediate N111 (200 mg, 0.48 mmol) and 4-chloro-3-fluoroaniline. Purification by column chromatography on silica gel (using a gradient of 0-100% EtOAc-EtOH 3:1 in isohexane as eluent) afforded the title compound as a beige solid (127 mg, yield: 56%). LC-MS: m/z [M+H]⁺: 455.2, 457.0; purity: 96%. ¹H NMR (400 MHz, DMSO-d₆) δ 10.52 (s, 1H), 8.54 (dd, J=4.7, 1.8 Hz, 1H), 8.24 (dd, J=8.0, 1.8 Hz, 1H), 7.73 (dd, J=11.9, 2.4 Hz, 1H), 7.52 (t, J=8.7 Hz, 1H), 7.38 (dd, J=8.0, 4.7 Hz, 1H), 7.30 (dt, J=8.8, 1.5 Hz, 1H), 6.39 (s, 1H), 4.49-4.36 (m, 2H), 3.58 (d, J=6.7 Hz, 1H), 3.51 (s, 3H), 2.43 (s, 3H), 1.48 (d, J=6.7 Hz, 3H). ¹⁹F NMR (376 MHz, DMSO-d₆) δ−114.69.

### Example #256: N-[4-(difluoromethyl)phenyl]-2-(9-fluoro-2,3,7-trimethyl-1,6-dioxo-7H-pyrido[4,3-d][3]benzazepin-5-yl)acetamide

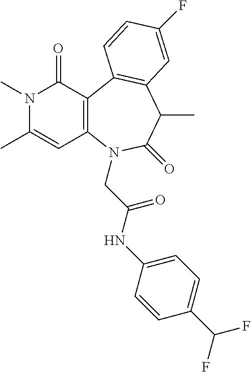 [SMILES: Cc1cc2c(c(=O)n1C)-c1ccc(F)cc1C(C)C(=O)N2CC(=O)Nc1ccc(C(F)F)cc1]

The title product was prepared according to the procedure described for example #128, starting from Intermediate N70 (125 mg, 0.34 mmol) and 4-(difluoromethyl)aniline. Purification by column chromatography on silica gel (using a gradient of 0-100% EtOAc-EtOH 3:1 in isohexane as eluent) afforded the title compound (106 mg, yield: 65%) as off white solid. LC-MS: m/z [M+H]⁺: 470.2; purity: 100%. ¹H NMR (400 MHz, DMSO-d₆) δ 10.39 (s, 1H), 7.88 (dd, J=8.8, 6.1 Hz, 1H), 7.69 (d, J=8.4 Hz, 2H), 7.51 (d, J=8.4 Hz, 2H), 7.17 (td, J=8.7, 2.7 Hz, 1H), 7.08 (dd, J=10.2, 2.4 Hz, 1H), 6.89 (t, J=56.1 Hz, 1H), 6.35 (s, 1H), 4.40 (d, J=2.0 Hz, 2H), 3.50 (s, 3H), 3.43 (q, J=6.8 Hz, 1H), 2.42 (s, 3H), 1.43 (d, J=6.8 Hz, 3H).

### Example #257: Enantiomer (10S) or (10R) of N-[4-(difluoromethyl)phenyl]-2-[3-fluoro-5,10-dimethyl-9-oxo-4,8,12,13-tetrazatricyclo[9.4.0.0^{2,7}]pentadeca-1(15),2,4,6,11,13-hexaen-8-yl]acetamide

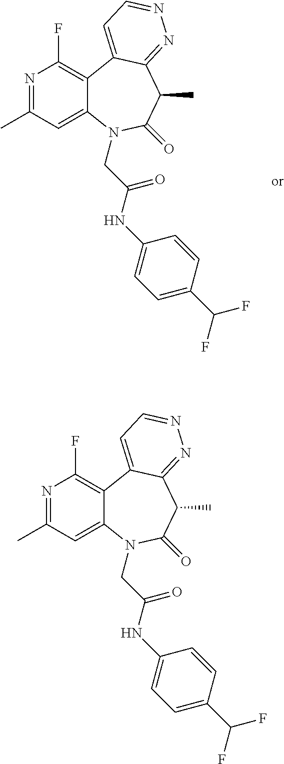 [SMILES: Cc1cc2c(c(F)n1)-c1ccnnc1[C@@H](C)C(=O)N2CC(=O)Nc1ccc(C(F)F)cc1 ; Cc1cc2c(c(F)n1)-c1ccnnc1[C@H](C)C(=O)N2CC(=O)Nc1ccc(C(F)F)cc1]

The title product was prepared according to the general procedure 1, starting from Intermediate N56 (140 mg, 0.45 mmol) and 2-chloro-N-[4-(difluoromethyl)phenyl]acetamide (CAS 872533-93-2). The racemate (49 mg) was separated by chiral HPLC with column CHIRALPAK AD 5 μm (20×250 mm) from Daicel, eluent EtOH:n-heptane 1:1+0.1% DEA, at 20 mL/min and RT to yield the enriched enantiomer as a white solid (12 mg, yield: 24%). Chiral purity: 74%; rt=3.82 min (second eluting enantiomer). For information, first eluting enantiomer rt=2.20 min. Both measured by chiral HPLC with column CHIRALPAK AD-3 (4.6×150 mm) from Daicel, eluent EtOH:n-heptane 1:1+0.1% DEA, at 1.5 mL/min and 30° C. LC-MS m/z [M+H]⁺: 442.0; purity: 96%. ¹H NMR (400 MHz, DMSO-d₆) δ 10.47 (s, 1H), 9.36 (d, J=5.3 Hz, 1H), 8.03 (t, J=4.8 Hz, 1H), 7.67 (d, J=8.4 Hz, 2H), 7.51 (d, J=8.3 Hz, 2H), 7.43 (s, 1H), 6.96 (t, J=55.7 Hz, 1H), 4.68-4.55 (m, 2H), 4.08 (q, J=6.5 Hz, 1H), 2.5 (s, 3H, hidden by DMSO-d₆), 1.63 (d, J=6.6 Hz, 3H).

### Example #258: Enantiomer (10S) or (10R) of 2-[3-chloro-5,10-dimethyl-9-oxo-4,8,12,13-tetrazatricyclo[9.4.0.0^{2,7}]pentadeca-1(15),2,4,6,11,13-hexaen-8-yl]-N-(4-chloro-3-fluoro-phenyl)acetamide

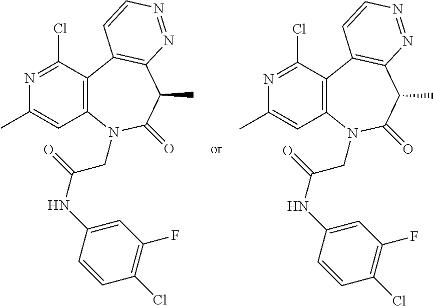

### Step 1: Synthesis of N-(4-chloro-3-fluoro-phenyl)-2-(3-fluoro-5,10-dimethyl-9-oxo-4,8,12,13-tetrazatricyclo[9.4.0.0^{2,7}]pentadeca-1(11),2,4,6,12,14-hexaen-8-yl)acetamide #258_1

The title product was prepared according to the general procedure 1, starting from Intermediate N56 (206 mg, 0.60 mmol), and 2-chloro-N-(4-chloro-3-fluoro-phenyl)acetamide (CAS 895641-02-8). The crude product was purified by column chromatography on silica gel (using a gradient of 0-100% EtOAc-EtOH 3:1 in isohexane as eluant) to afford the title compound as a beige solid (215 mg, yield: 77%). LC-MS m/z [M+H]⁺: 444.2, 446.2; purity: 95%.

### Step 2: Synthesis of 2-(3-chloro-5,10-dimethyl-9-oxo-4,8,12,13-tetrazatricyclo[9.4.0.0^{2,7}]pentadeca-1(11),2,4,6,12,14-hexaen-8-yl)-N-(4-chloro-3-fluoro-phenyl)acetamide #258

A mixture of N-(4-chloro-3-fluoro-phenyl)-2-(3-fluoro-5,10-dimethyl-9-oxo-4,8,12,13-tetrazatricyclo[9.4.0.0^{2,7}]pentadeca-1(11),2,4,6,12,14-hexaen-8-yl)acetamide #258_1 (0.20 g, 0.43 mmol) and HCl (4N in 1,4-dioxane) (3.75 mL, 15.0 mmol) was heated at 100° C. in a sealed microwave vial for 60 minutes. The reaction mixture was allowed to cool to RT and then the crude reaction mixture carefully poured onto saturated NaHCO₃ solution (100 mL). The product was extracted into EtOAc (2×40 mL) and the organics were combined, dried over Na₂SO₄, filtered and concentrated under vacuo. After purification by column chromatography, the solid (racemate, 101 mg) was separated by chiral SFC with column Chiralpak AD 20 μm (50×279 mm) from Daicel, eluent CO₂+30% EtOH, at 360 mL/min, 35° C. and BPR 150 bar to yield the pure enantiomer as a white solid (46 mg, yield: 46%). Chiral purity: 98%; rt=2.69 min (second eluting enantiomer). For information, first eluting enantiomer rt=1.90 min. Both measured by chiral HPLC with column Chiralpak IB-3 (4.6×150 mm) from Daicel, eluent Acetonitrile 100%+0.1% DEA, at 1.5 mL/min and 30° C. LC-MS m/z [M+H]⁺: 459.9; purity: 100%. ¹H NMR (400 MHz, DMSO-d₆) δ 10.55 (s, 1H), 9.37 (d, J=5.4 Hz, 1H), 8.16 (d, J=5.3 Hz, 1H), 7.69 (dd, J=11.8, 2.4 Hz, 1H), 7.55-7.46 (m, 2H), 7.28 (ddd, J=8.8, 2.5, 1.0 Hz, 1H), 4.66-4.46 (m, 2H), 4.12 (q, J=6.6 Hz, 1H), 2.55 (s, 3H), 1.62 (d, J=6.6 Hz, 3H).

### Example #259: Enantiomer (10S) or (10R) of N-[4-(difluoromethyl)phenyl]-2-[3-fluoro-5,10-dimethyl-9-oxo-4,8,14-triazatricyclo[9.4.0.0^{2,7}]pentadeca-1(15),2,4,6,11,13-hexaen-8-yl]acetamide

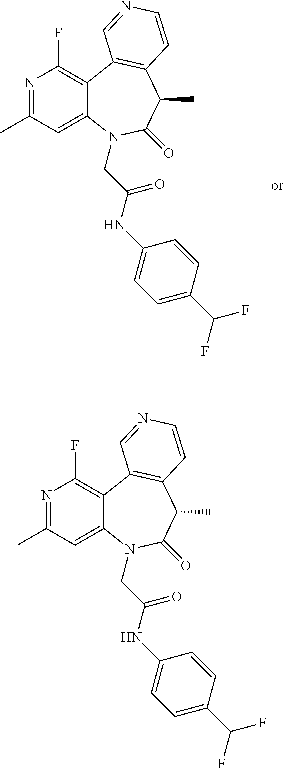 [SMILES: Cc1cc2c(c(F)n1)-c1cnccc1[C@@H](C)C(=O)N2CC(=O)Nc1ccc(C(F)F)cc1 ; Cc1cc2c(c(F)n1)-c1cnccc1[C@H](C)C(=O)N2CC(=O)Nc1ccc(C(F)F)cc1]

The title product was prepared according to the general procedure 1, starting from Intermediate N57 (74.0 mg, 0.27 mmol) and 2-chloro-N-[4-(difluoromethyl)phenyl]acetamide (CAS 872533-93-2). The racemate was separated by Chiral SFC (column CHIRALPAK IA from Daicel, eluant 45% MeOH, 55% CO₂) to yield the pure enantiomer as a pale yellow solid (30 mg, yield: 37%). LC-MS m/z [M+H]⁺: 441.2; purity: 100%. ¹H NMR (400 MHz, DMSO-d₆) δ 10.46 (s, 1H), 8.87 (d, J=5.2 Hz, 1H), 8.67 (d, J=5.2 Hz, 1H), 7.69 (d, J=8.3 Hz, 2H), 7.51 (d, J=8.3 Hz, 2H), 7.45 (d, J=5.3 Hz, 1H), 7.37 (s, 1H), 6.96 (t, J=56.1 Hz, 1H), 4.59 (d, J=16.9 Hz, 1H), 4.54 (d, J=16.7 Hz, 1H), 3.68 (q, J=6.6 Hz, 1H), 1.47 (d, J=6.7 Hz, 3H), three protons obscured under the solvent peak. ¹⁹F NMR (376 MHz, DMSO-d₆) δ−70.91, −108.11. Chiral purity: 100%; rt=4.28 min (second eluting enantiomer). For information, first eluting enantiomer rt=1.24 min. Both measured by SFC, CHIRALPAK IA from Daicel, 55% CO₂ and 45% MeOH+0.1% NH₃.

### Example #260: N-(4-chloro-3-fluoro-phenyl)-2-[(10S)-3,14-difluoro-5,10-dimethyl-9-oxo-4,8,12-triazatricyclo[9.4.0.0^{2,7}]pentadeca-1(11),2(7),3,5,12,14-hexaen-8-yl]acetamide

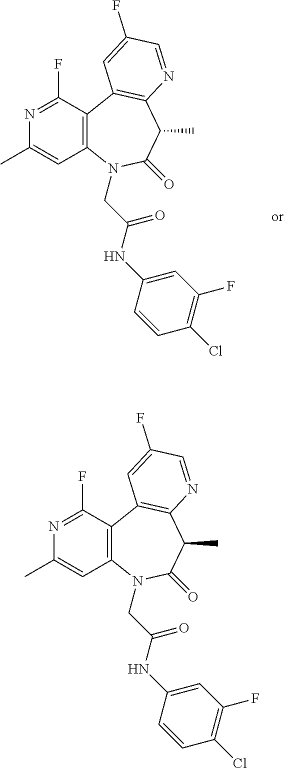 [SMILES: Cc1cc2c(c(F)n1)-c1cc(F)cnc1[C@H](C)C(=O)N2CC(=O)Nc1ccc(Cl)c(F)c1 ; Cc1cc2c(c(F)n1)-c1cc(F)cnc1[C@@H](C)C(=O)N2CC(=O)Nc1ccc(Cl)c(F)c1]

The title product was prepared according to the general procedure 1, starting from 3,14-difluoro-5,10-dimethyl-4,8,12-triazatricyclo[9.4.0.0^{2,7}]pentadeca-1(11),2(7),3,5,12,14-hexaen-9-one NN142 (1.50 g, 5.45 mmol) and 2-chloro-N-(4-chloro-3-fluoro-phenyl)acetamide (1.57 g, 7.07 mmol). The racemate residue was separated by chiral chromatography (SFC CHIRALPAK IG from Daicel, eluant: CO₂+iPrOH 20%) to afford the title compound as a white solid (942.9 mg, yield: 38%). Chiral purity: 100%; rt=1.80 min (second eluting enantiomer). For information, first eluting enantiomer rt=1.46 min. Both measured by HPLC (CHIRALPAK IA from Daicel, eluant: EtOH:Heptane 50%—DEA 0.1%). LC-MS m/z [M+H]⁺: 461.1/463.1; purity: 99%. ¹H NMR (400 MHz, DMSO-d₆) δ 10.60 (s, 1H), 8.71 (d, J=2.8 Hz, 1H), 8.18-8.10 (m, 1H), 7.73 (dd, J=11.8, 2.4 Hz, 1H), 7.53 (t, J=8.7 Hz, 1H), 7.38 (s, 1H), 7.31 (dd, J=8.7, 2.4 Hz, 1H), 4.57 (s, 2H), 3.81 (q, J=6.5 Hz, 1H), 1.48 (d, J=6.5 Hz, 3H) (+one CH₃ present under the signal of the DMSO-d₆). ¹⁹F NMR (376 MHz, DMSO-d₆) δ−70.61 (d, J=4.0 Hz), −114.63 (dd, J=11.8, 8.7 Hz), −130.19 (d, J=9.6 Hz).

### Example #261: Enantiomer (10S) or (10R) of N-[4-(difluoromethyl)phenyl]-2-[3,14-difluoro-5,10-dimethyl-9-oxo-4,8,12-triazatricyclo[9.4.0.0^{2,7}]pentadeca-1(11),2(7),3,5,12,14-hexaen-8-yl]acetamide

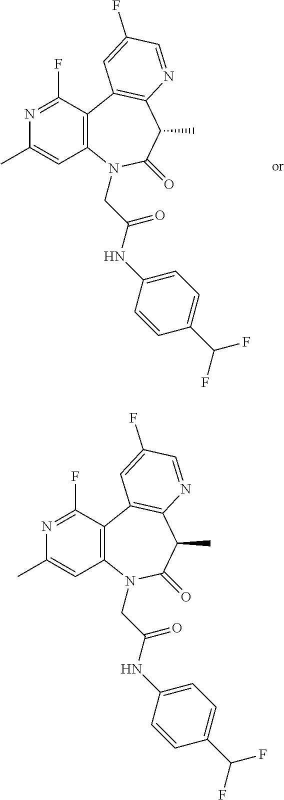 [SMILES: Cc1cc2c(c(F)n1)-c1cc(F)cnc1[C@H](C)C(=O)N2CC(=O)Nc1ccc(C(F)F)cc1 ; Cc1cc2c(c(F)n1)-c1cc(F)cnc1[C@@H](C)C(=O)N2CC(=O)Nc1ccc(C(F)F)cc1]

The title product was prepared according to the general procedure 1, starting from 3,14-difluoro-5,10-dimethyl-4,8,12-triazatricyclo[9.4.0.0^{2,7}]pentadeca-1(11),2(7),3,5,12,14-hexaen-9-one NN142 (500.0 mg, 1.817 mmol) and 2-chloro-N-[4-(difluoromethyl)phenyl]acetamide (479.0 mg, 2.181 mmol). The racemate residue was separated by chiral chromatography (SFC Reprosil Chiral NR-R from Dr Maisch, eluant: CO₂+iPrOH 25%) to afford the title compound as a white solid (260.1 mg, yield: 39%). Chiral purity: 100%; rt=2.96 min (second eluting enantiomer). For information, first eluting enantiomer rt=1.51 min. Both measured by HPLC (Reprosil Chiral NR-R from Dr Maisch, eluant: EtOH:Heptane 50%—DEA 0.1%). LC-MS m/z [M+H]⁺: 459; purity: 99%. ¹H NMR (400 MHz, DMSO-d₆) δ 10.48 (s, 1H), 8.71 (d, J=2.7 Hz, 1H), 8.19-8.10 (m, 1H), 7.69 (d, J=8.3 Hz, 2H), 7.52 (d, J=8.3 Hz, 2H), 7.40 (s, 1H), 6.97 (t, J=56.1 Hz, 1H), 4.59 (s, 2H), 3.81 (q, J=6.6 Hz, 1H), 1.48 (d, J=6.6 Hz, 3H) (+one CH₃ present under the signal of the DMSO-d₆). ¹⁹F NMR (376 MHz, DMSO-d₆) δ−70.63 (d, J=4.8 Hz), −108.08 (d, J=56.1 Hz), −130.21 (d, J=9.8 Hz).

### Example #262: Enantiomer (10S) or (10R) of 2-[3,14-difluoro-5,10-dimethyl-9-oxo-4,8,12-triazatricyclo[9.4.0.0^{2,7}]pentadeca-1(11),2(7),3,5,12,14-hexaen-8-yl]-N-[6-(trifluoromethyl)-3-pyridyl]acetamide

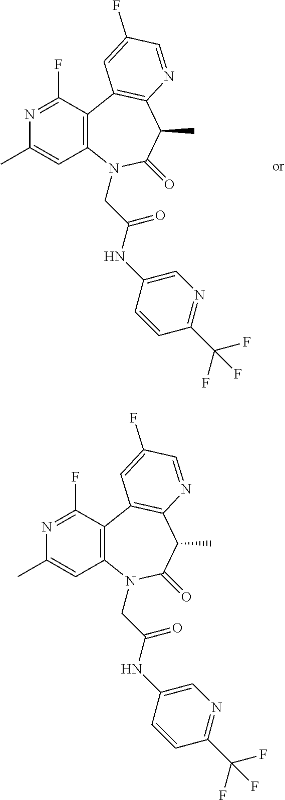 [SMILES: Cc1cc2c(c(F)n1)-c1cc(F)cnc1[C@@H](C)C(=O)N2CC(=O)Nc1ccc(C(F)(F)F)nc1 ; Cc1cc2c(c(F)n1)-c1cc(F)cnc1[C@H](C)C(=O)N2CC(=O)Nc1ccc(C(F)(F)F)nc1]

The title product was prepared according to the general procedure 1, starting from 3,14-difluoro-5,10-dimethyl-4,8,12-triazatricyclo[9.4.0.0^{2,7}]pentadeca-1(11),2(7),3,5,12,14-hexaen-9-one NN142 (130.0 mg, 0.4724 mmol) and 2-chloro-N-[6-(trifluoromethyl)-3-pyridyl]acetamide (154.0 mg, 0.613 mmol). The racemate residue was separated by chiral chromatography (SFC CHIRALPAK IA from Daicel, eluant: CO₂+EtOH 20%) to afford the title compound as a white solid (51.2 mg, yield: 23%). Chiral purity: 99%; rt=2.09 min (second eluting enantiomer). For information, first eluting enantiomer rt=1.51 min. Both measured by HPLC (CHIRALPAK IA from Daicel, eluant: EtOH:Heptane 50%—DEA 0.1%). LC-MS m/z [M+H]⁺: 478.1; purity: 99%. ¹H NMR (500 MHz, DMSO-d₆) δ 10.88 (s, 1H), 8.83 (d, J=2.4 Hz, 1H), 8.71 (d, J=2.8 Hz, 1H), 8.29 (dd, J=8.6, 2.4 Hz, 1H), 8.17-8.12 (m, 1H), 7.87 (d, J=8.6 Hz, 1H), 7.39 (s, 1H), 4.63 (s, 2H), 3.82 (q, J=6.6 Hz, 1H), 1.48 (d, J=6.6 Hz, 3H) (+one CH₃ present under the signal of the DMSO-d₆). ¹⁹F NMR (471 MHz, DMSO-d₆) δ−65.67, −70.55 (d, J=4.3 Hz), −130.20 (d, J=8.8 Hz).

### Example #263: Enantiomer (10S) or (10R) of 2-[3,14-difluoro-5,10-dimethyl-9-oxo-4,8,12-triazatricyclo[9.4.0.0^{2,7}]pentadeca-1(15),2,4,6,11,13-hexaen-8-yl]-N-[4-(1,1-difluoroethyl)phenyl]acetamide

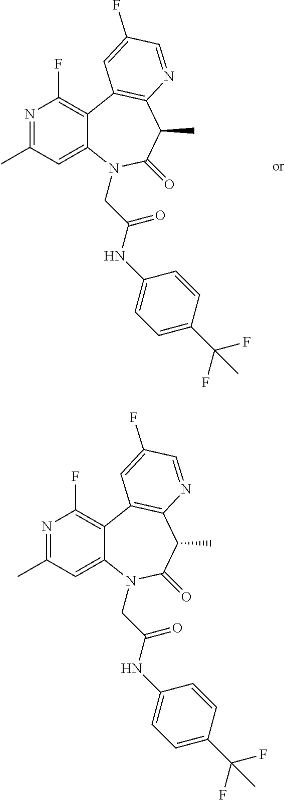 [SMILES: Cc1cc2c(c(F)n1)-c1cc(F)cnc1[C@@H](C)C(=O)N2CC(=O)Nc1ccc(C(C)(F)F)cc1 ; Cc1cc2c(c(F)n1)-c1cc(F)cnc1[C@H](C)C(=O)N2CC(=O)Nc1ccc(C(C)(F)F)cc1]

The title product was prepared according to the general procedure 1, starting from 3,14-difluoro-5,10-dimethyl-4,8,12-triazatricyclo[9.4.0.0^{2,7}]pentadeca-1(11),2(7),3,5,12,14-hexaen-9-one NN142 (165 mg, 0.510 mmol) and 2-chloro-N-[4-(1,1-difluoroethyl)phenyl]acetamide (CAS 872533-93-2). The racemate was separated by chiral SFC (CHIRALPAK IA from Daicel, eluent 35% MeOH, 65% CO₂) to yield the pure enantiomer (29 mg, yield: 12%). LC-MS m/z [M+H]⁺: 473.2; purity: 99%. ¹H NMR (400 MHz, DMSO-d₆) δ 10.42 (s, 1H), 8.70 (d, J=2.8 Hz, 1H), 8.14 (dq, J=9.7, 3.0 Hz, 1H), 7.65 (d, J=8.5 Hz, 2H), 7.50 (d, J=8.5 Hz, 2H), 7.38 (s, 1H), 4.58 (s, 2H), 3.80 (q, J=6.6 Hz, 1H), 2.49 (s, 3H), 1.94 (t, J=18.8 Hz, 3H), 1.48 (d, J=6.6 Hz, 3H). ¹⁹F NMR (376 MHz, DMSO-d₆) δ−70.61, −83.12, −130.23. Chiral purity: 99.4%; rt=2.12 min (second eluting enantiomer). For information, first eluting enantiomer rt=0.95 min. Both measured by chiral SFC column CHIRALPAK IA from Daicel, eluent 50% MeOH+0.1% NH₃, 50% CO₂.

### Example #264: N-(4-chloro-3-fluoro-phenyl)-2-[3-fluoro-5-[2-methoxyethyl(methyl)amino]-10-methyl-9-oxo-4,8,12-triazatricyclo[9.4.0.0^{2,7}]pentadeca-1(15),2(7),3,5,11,13-hexaen-8-yl]acetamide

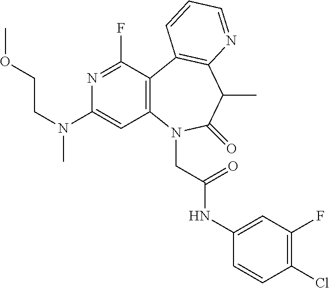 [SMILES: COCCN(C)c1cc2c(c(F)n1)-c1cccnc1C(C)C(=O)N2CC(=O)Nc1ccc(Cl)c(F)c1]

### General Procedure SNAr_A for Aromatic Nucleophilic Substitutions

Sodium hydride (20 mg, 0.25 mmol) was added at room temperature to a solution of N-(4-chloro-3-fluoro-phenyl)-2-(3,5-difluoro-10-methyl-9-oxo-4,8,12-triazatricyclo[9.4.0.0^{2,7}]pentadeca-1(11),2(7),3,5,12,14-hexaen-8-yl)acetamide NN209a (100 mg, 0.22 mmol) and N-(2-methoxyethyl)methylamine (22 mg, 0.32 mmol) in THF (1 mL). The reaction mixture was stirred at room temperature for 16 h. The reaction mixture was diluted with EtOAc (25 mL) and the resulting mixture was washed with Brine (25 mL), dried over MgSO₄, filtered and concentrated under vacuo to afford 116 mg of white solid. The crude was purified by reverse phase chromatography (Method G_B, 140 mL/min, gradient from 5% ACN to 90% ACN and 10% basic modifier) to afford the title compound as a white solid (12 mg, yield: 10%). LC-MS m/z [M+H]⁺: 516.2; purity: 98%.

Similar general procedures related to SNAr_A will be used in the subsequent examples. General procedure SNAr_B (in ACN and in the presence of 3 eq. of DIPEA at RT), General procedure SNAr_C (EtOH, 90° C.), General procedure SNAr_D (5 eq. of K₂CO₃ in DMSO at 70° C.) and General procedure SNAr_E (NaH in THF at 70° C.).

### Example #265: Enantiomer (10S) or (10R) of N-(4-chloro-3-fluoro-phenyl)-2-[3-fluoro-5-[2-methoxyethyl(methyl)amino]-10-methyl-9-oxo-4,8,12-triazatricyclo[9.4.0.0^{2,7}]pentadeca-1(15),2(7),3,5,11,13-hexaen-8-yl]acetamide

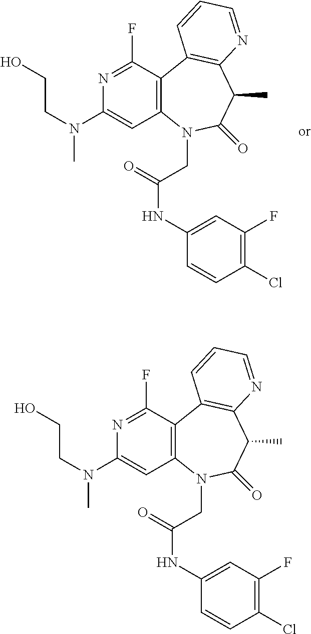 [SMILES: C[C@H]1C(=O)N(CC(=O)Nc2ccc(Cl)c(F)c2)c2cc(N(C)CCO)nc(F)c2-c2cccnc21 ; C[C@@H]1C(=O)N(CC(=O)Nc2ccc(Cl)c(F)c2)c2cc(N(C)CCO)nc(F)c2-c2cccnc21]

The title product was prepared according to general procedure SnAr_C using 2-(methylamino)ethanol and N-(4-chloro-3-fluoro-phenyl)-2-(3,5-difluoro-10-methyl-9-oxo-4,8,12-triazatricyclo[9.4.0.0^{2,7}]pentadeca-1(11),2(7),3,5,12,14-hexaen-8-yl)acetamide NN209a. The white solid (racemate) was separated by chiral HPLC with column Reprosil NR-R chiral 5 μm (20×250 mm) from Dr Maisch, eluent iPrOH:n-heptane 1:1+0.1% DEA, at 20 mL/min and RT to yield the pure enantiomer as a white solid (6 mg, yield: 5%). Chiral purity: 100%; rt=2.51 min (first eluting enantiomer). For information, second eluting enantiomer rt=3.16 min. Both measured by chiral HPLC with column Reprosil NR-R chiral 3 μm (4.6×150 mm) from Dr Maisch, eluent iPrOH:n-heptane 1:1+0.1% DEA, at 1.5 mL/min and 30° C. LC-MS m/z [M+H]⁺: 502.0; purity: 99%. ¹H NMR (400 MHz, DMSO-d₆) δ 10.55 (s, 1H), 8.55 (dd, J=4.9, 1.7 Hz, 1H), 7.98 (td, J=5.1, 2.6 Hz, 1H), 7.70 (dd, J=11.8, 2.4 Hz, 1H), 7.51 (t, J=8.7 Hz, 1H), 7.43 (dd, J=7.9, 4.8 Hz, 1H), 7.30 (dd, J=8.7, 2.3 Hz, 1H), 6.54 (s, 1H), 4.75 (s, 1H), 4.63-4.49 (m, 2H), 3.79 (q, J=6.6 Hz, 1H), 3.59 (s, 4H), 3.07 (s, 3H), 1.46 (d, J=6.7 Hz, 3H).

### Example #266: Enantiomer (10S) or (10R) of N-(4-chloro-3-fluoro-phenyl)-2-[5-[2-(dimethylamino)ethyl-methyl-amino]-3-fluoro-10-methyl-9-oxo-4,8,12-triazatricyclo[9.4.0.0^{2,7}]pentadeca-1(15),2,4,6,11,13-hexaen-8-yl]acetamide

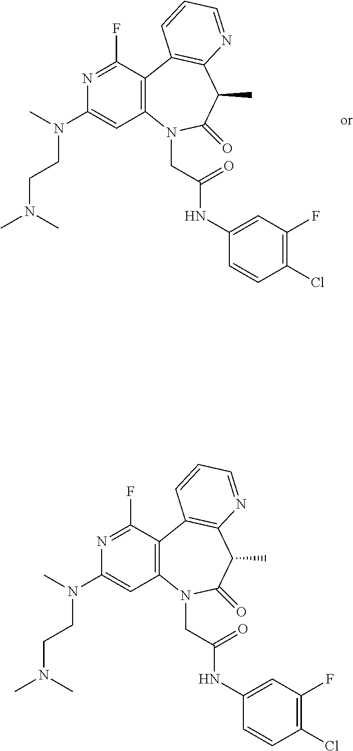 [SMILES: C[C@H]1C(=O)N(CC(=O)Nc2ccc(Cl)c(F)c2)c2cc(N(C)CCN(C)C)nc(F)c2-c2cccnc21 ; C[C@@H]1C(=O)N(CC(=O)Nc2ccc(Cl)c(F)c2)c2cc(N(C)CCN(C)C)nc(F)c2-c2cccnc21]

The title product was prepared according to general procedure SnAr_C using N-(4-chloro-3-fluoro-phenyl)-2-(3,5-difluoro-10-methyl-9-oxo-4,8,12-triazatricyclo[9.4.0.0^{2,7}]pentadeca-1(11),2(7),3,5,12,14-hexaen-8-yl)acetamide NN209a and N,N,N′-trimethyletylenediamine. The withe solid (racemate) was separated by chiral HPLC with column Reprosil NR-R chiral 5 μm (20×250 mm) from Dr Maisch, eluent iPrOH:n-heptane 1:1+0.1% DEA, at 20 mL/min and RT to yield the pure enantiomer as a white solid (5 mg, yield: 4%). Chiral purity: 100%; rt=3.04 min (first eluting enantiomer). For information, second eluting enantiomer rt=4.10 min. Both measured by by chiral HPLC with column Reprosil NR-R chiral 3 μm (4.6×150 mm) from Dr Maisch, eluent iPrOH:n-heptane 1:1+0.1% DEA, at 1.5 mL/min and 30° C. In order to remove small impurities, an additional purification of the enantiomer was needed (Purification Method P_B) to yield the title compounds as a white solid (1.8 mg). LC-MS m/z [M+H]⁺: 529.24; purity: 95%.

### Example #267: Enantiomer (10S) or (10R) of 2-[5-(5-azaspiro[2.3]hexan-5-yl)-3-fluoro-10-methyl-9-oxo-4,8,12-triazatricyclo[9.4.0.0^{2,7}]pentadeca-1(15),2,4,6,11,13-hexaen-8-yl]-N-(4-chloro-3-fluoro-phenyl)acetamide

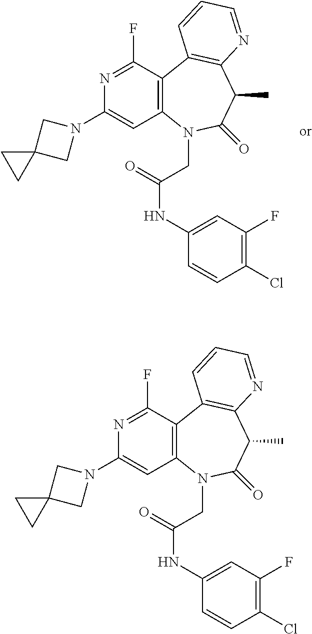 [SMILES: C[C@H]1C(=O)N(CC(=O)Nc2ccc(Cl)c(F)c2)c2cc(N3CC4(CC4)C3)nc(F)c2-c2cccnc21 ; C[C@@H]1C(=O)N(CC(=O)Nc2ccc(Cl)c(F)c2)c2cc(N3CC4(CC4)C3)nc(F)c2-c2cccnc21]

The title product was prepared according to general procedure SnAr_B using 5-azaspiro[2.3]hexane ethanedioate and N-(4-chloro-3-fluoro-phenyl)-2-[(10R)-3,5-difluoro-10-methyl-9-oxo-4,8,12-triazatricyclo[9.4.0.0^{2,7}]pentadeca-1(11),2(7).3,5,12,14-hexaen-8-yl]acetamide NN209b. The crude mixture was purified by reverse phase chromatography (Purification Method P_A) to afford the title compound as a white solid (73 mg, yield: 64%). LC-MS m/z [M+H]⁺: 510.06; purity: 97%. ¹H NMR (400 MHz, DMSO-d₆) δ 10.55 (s, 1H), 8.58-8.55 (dd, J=4.8, 1.7 Hz, 1H), 8.03-7.96 (m, 1H), 7.71 (dd, J=7.4, 2.3 Hz, 1H), 7.52 (t, J=8.7 Hz, 1H), 7.43 (dd, J=7.9, 4.8 Hz, 1H), 7.33-7.26 (m, 1H), 6.33 (s, 1H), 5.76 (s, 1H), 4.61-4.47 (m, 2H), 4.16-4.05 (m, 4H), 3.81 (q, J=6.5 Hz, 1H), 1.47 (d, J=6.7 Hz, 3H), 0.75-0.62 (m, 3H).

### Example #268: Enantiomer (10S) or (10R) of N-(4-chloro-3-fluoro-phenyl)-2-[5-(3,3-difluoroazetidin-1-yl)-3-fluoro-10-methyl-9-oxo-4,8,12-triazatricyclo[9.4.0.0^{2,7}]pentadeca-1(15),2,4,6,11,13-hexaen-8-yl]acetamide

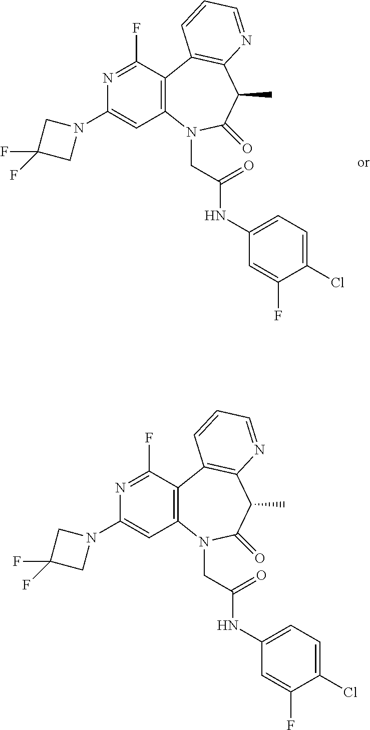 [SMILES: C[C@H]1C(=O)N(CC(=O)Nc2ccc(Cl)c(F)c2)c2cc(N3CC(F)(F)C3)nc(F)c2-c2cccnc21 ; C[C@@H]1C(=O)N(CC(=O)Nc2ccc(Cl)c(F)c2)c2cc(N3CC(F)(F)C3)nc(F)c2-c2cccnc21]

The title product was prepared according to general procedure SnAr_D using N-(4-chloro-3-fluoro-phenyl)-2-(3,5-difluoro-10-methyl-9-oxo-4,8,12-triazatricyclo[9.4.0.0^{2,7}]pentadeca-1(11),2(7),3,5,12,14-hexaen-8-yl)acetamide NN209a and 3,3-difluoroazetidine hydrochloride. The racemic residue was purified by SFC chromatography (column GreenSep Nitro 5p 120A, using MeOH as solvent from 5% to 50%) followed by chiral chromatography (Reprosil Chiral NR-R, using Heptane-Ethanol 1:1 as solvents) to give the compound #268 as a white solid (69 mg, Yield: 20%) and the other enantiomer also as white solid (64.6 mg, Yield=18.5%). LC-MS m/z [M+H]⁺: 520; purity: 99%. LC-MS (acid, method A2) m/z [M+H]⁺: 520; rt: 4.79 min; purity: 90%. ¹H NMR (400 MHz, DMSO-d₆) δ 10.56 (s, 1H), 8.60 (dd, J=4.7, 1.6 Hz, 1H), 8.02 (ddd, J=7.8, 4.6, 1.7 Hz, 1H), 7.71 (dd, J=11.9, 2.4 Hz, 1H), 7.52 (t, J=8.7 Hz, 1H), 7.45 (dd, J=7.9, 4.8 Hz, 1H), 7.31-7.27 (m, 1H), 6.53 (s, 1H), 4.67-4.40 (m, 6H), 3.80 (q, J=6.5 Hz, 1H), 1.47 (d, J=6.6 Hz, 3H). Chiral purity: 100%; rt=2.19 min (second eluting enantiomer) column CHIRAL NR-R, using Heptane-Ethanol 1:1 as solvents with 0.5% DEA. For information, first eluting enantiomer rt 1.92 min. Both measured by HPLC, Chiral NR-R, EtOH 50%—heptane 50%—DEA 0.5%.

### Example #269: Enantiomer (10S) or (10R) of 2-[5-(6-azabicyclo[3.1.1]heptan-6-yl)-3-fluoro-10-methyl-9-oxo-4,8,12-triazatricyclo[9.4.0.0^{2,7}]pentadeca-1(11),2(7),3,5,12,14-hexaen-8-yl]-N-(4-chloro-3-fluoro-phenyl)acetamide

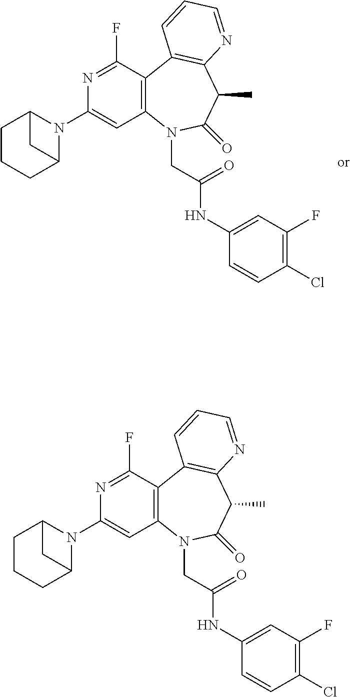 [SMILES: C[C@H]1C(=O)N(CC(=O)Nc2ccc(Cl)c(F)c2)c2cc(N3C4CCCC3C4)nc(F)c2-c2cccnc21 ; C[C@@H]1C(=O)N(CC(=O)Nc2ccc(Cl)c(F)c2)c2cc(N3C4CCCC3C4)nc(F)c2-c2cccnc21]

The title product was prepared according to general procedure SnAr_B using 6-azabicyclo[3.1.1]heptane-hydrochloride and N-(4-chloro-3-fluoro-phenyl)-2-[(10R)-3,5-difluoro-10-methyl-9-oxo-4,8,12-triazatricyclo[9.4.0.0^{2,7}]pentadeca-1(11),2(7),3,5,12,14-hexaen-8-yl]acetamide NN209b. The crude mixture was purified by reverse phase chromatography (Purification Method P_A) to afford the title compound as a white solid (43 mg, yield: 37%). LC-MS m/z [M+H]⁺: 524.16; purity: 93%.

### Example #270: Enantiomer (10S) or (10R) of N-(4-chloro-3-fluoro-phenyl)-2-[5-(2,2-difluoro-5-azaspiro[2.3]hexan-5-yl)-3-fluoro-10-methyl-9-oxo-4,8,12-triazatricyclo[9.4.0.0^{2,7}]pentadeca-1(15),2,4,6,11,13-hexaen-8-yl]acetamide

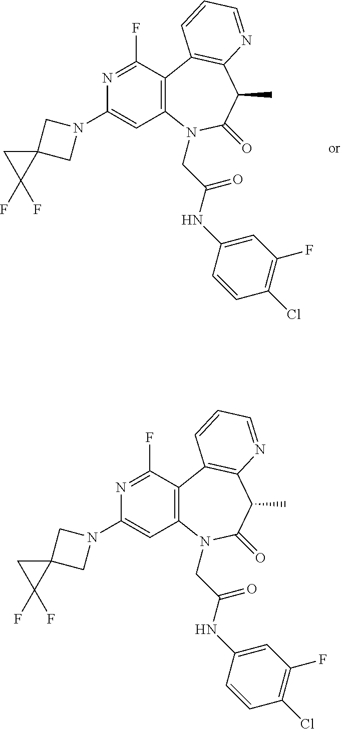 [SMILES: C[C@H]1C(=O)N(CC(=O)Nc2ccc(Cl)c(F)c2)c2cc(N3CC4(C3)CC4(F)F)nc(F)c2-c2cccnc21 ; C[C@@H]1C(=O)N(CC(=O)Nc2ccc(Cl)c(F)c2)c2cc(N3CC4(C3)CC4(F)F)nc(F)c2-c2cccnc21]

The title product was prepared according to general procedure SnAr_B using N-(4-chloro-3-fluoro-phenyl)-2-(3,5-difluoro-10-methyl-9-oxo-4,8,12-triazatricyclo[9.4.0.0^{2,7}]pentadeca-1(11),2(7),3,5,12,14-hexaen-8-yl)acetamide NN209a and 1,1-difluoro-5-azaspiro[2.3]hexane. The racemic residue was purified by SFC chromatography (DC4 P4VP from Daicel, using EtOH as solvent from 5% to 50%) followed by chiral chromatography (Reprosil CHIRAL NR-R from Dr Maisch, using Heptane-IPA 1:1 as solvents) to give the compound as a white solid (14 mg, Yield: 14.3%) and the other enantiomer as white solid (17 mg, Yield: 17.4%). LC-MS m/z [M+H]⁺: 546; purity: 96%. Chiral purity: 100%; rt=3.16 min (first eluting enantiomer) (column CHIRAL NR-R, using Heptane-IPA 1:1 as solvents with 0.1% DEA). ¹H NMR (500 MHz, DMSO-d₆) δ 10.54 (s, 1H), 8.60-8.57 (m, 1H), 8.01 (dd, J=8.4, 4.1 Hz, 1H), 7.71 (d, J=11.5 Hz, 1H), 7.51 (t, J=8.6 Hz, 1H), 7.46-7.42 (m, 1H), 7.30 (d, J=8.9 Hz, 1H), 6.44 (s, 1H), 4.56 (q, J=16.6 Hz, 2H), 4.26-4.12 (m, 4H), 3.79 (dq, J=14.2, 6.0 Hz, 1H), 1.80 (p, J=8.9 Hz, 2H), 1.47 (d, J=6.6 Hz, 3H). For information, second eluting enantiomer rt=4.02 min (94%).

### Example #271: N-[4-(difluoromethyl)phenyl]-2-(3-fluoro-10-methyl-5-morpholino-9-oxo-4.8,12-triazatricyclo[9.4.0.0^{2,7}]pentadeca-1(15),2,4,6,11,13-hexaen-8-yl)acetamide

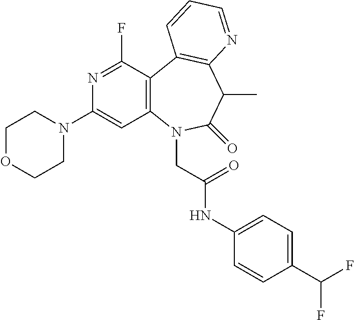 [SMILES: CC1C(=O)N(CC(=O)Nc2ccc(C(F)F)cc2)c2cc(N3CCOCC3)nc(F)c2-c2cccnc21]

The title product was prepared according to general procedure SnAr_E using 2-(3,5-difluoro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide NN230 and morpholine hydrochloride. The crude mixture was purified by two consecutive reverse phase chromatography (Purification Method P_B followed by Purification Method P_A) to afford the title compound as a white solid (6.5 mg, yield: 5%). LC-MS m/z [M+H]⁺: 512.28; purity: 97%.

### Example #272: N-[4-(difluoromethyl)phenyl]-2-[3-fluoro-5-[(2S)-2-(hydroxymethyl)azetidin-1-yl]-10-methyl-9-oxo-4,8,12-triazatricyclo[9.4.0.0^{2,7}]pentadeca-1(15),2,4,6,11,13-hexaen-8-yl]acetamide

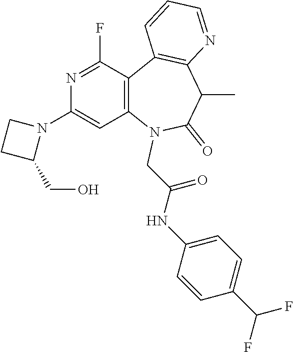 [SMILES: CC1C(=O)N(CC(=O)Nc2ccc(C(F)F)cc2)c2cc(N3CC[C@H]3CO)nc(F)c2-c2cccnc21]

The title product was prepared according to general procedure SnAr_B using 2-(3,5-difluoro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide NN230 and (S)-azetidin-2-ylmethanol. The residue was purified by SFC (Phenomenex Luna 5 μm Silica (2) 100A, CO₂+MeOH) to afford the title compound (27.1 mg, yield: 47%) as a white solid. LC-MS m/z: [M+H]⁺: 512.0; purity: 99%. ¹H NMR (400 MHz, DMSO-d₆) δ 10.41 (s, 1H), 8.56 (dd, J=4.8, 1.6 Hz, 1H), 8.03-7.92 (m, 1H), 7.66 (d, J=8.3 Hz, 2H), 7.51 (d, J=8.3 Hz, 2H), 7.43 (dd, J=7.9, 4.8 Hz, 1H), 6.96 (t, J=56.1 Hz, 1H), 6.46 (s, 1H), 4.94 (t, J=5.3 Hz, 1H), 4.58-4.48 (m, 2H), 4.40-4.26 (m, 1H), 3.95-3.83 (m, 2H), 3.83-3.75 (m, 1H), 3.75-3.60 (m, 2H), 2.37-2.27 (m, 1H), 2.26-2.15 (m, 1H), 1.47 (d, J=6.6 Hz, 3H). ¹⁹F NMR (376 MHz, DMSO-d₆) δ−72.22 (dd, J=17.5, 3.9 Hz), −108.06 (d, J=56.1 Hz).

### Example #273: N-[4-(difluoromethyl)phenyl]-2-[3-fluoro-5-[(2R)-2-(hydroxymethyl)azetidin-1-yl]-10-methyl-9-oxo-4,8,12-triazatricyclo[9.4.0.0^{2,7}]pentadeca-1(15),2,4,6,11,13-hexaen-8-yl]acetamide

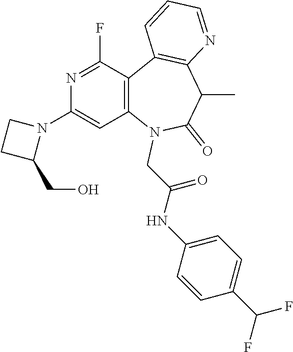 [SMILES: CC1C(=O)N(CC(=O)Nc2ccc(C(F)F)cc2)c2cc(N3CC[C@@H]3CO)nc(F)c2-c2cccnc21]

The title product was prepared according to general procedure SnAr_B using 2-(3,5-difluoro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide NN230 and (R)-azetidin-2-ylmethanol. The residue was purified by SFC (Phenomenex Luna 5 μm Silica (2) 100A, CO₂+MeOH) to afford the title compound (16.4 mg, yield: 30%) as a white solid. LC-MS m/z: [M+H]⁺: 512.0; purity: 100%. LC-MS (Method B2) m/z: [M+H]⁺: 512.1; rt: 3.96 min; purity: 100%. ¹H NMR (400 MHz, DMSO-d₆) δ 10.42 (s, 1H), 8.56 (dd, J=4.8, 1.6 Hz, 1H), 8.01-7.95 (m, 1H), 7.66 (d, J=8.3 Hz, 2H), 7.51 (d, J=8.3 Hz, 2H), 7.43 (dd, J=7.9, 4.8 Hz, 1H), 6.96 (t, J=56.1 Hz, 1H), 6.46 (s, 1H), 4.95 (s, 1H), 4.58-4.49 (m, 2H), 4.39-4.26 (m, 1H), 3.96-3.81 (m, 2H), 3.81-3.75 (m, 1H), 3.75-3.58 (m, 2H), 2.36-2.28 (m, 1H), 2.27-2.15 (m, 1H), 1.47 (d, J=6.6 Hz, 3H). ¹⁹F NMR (376 MHz, DMSO-d₆) δ−72.22 (dd, J=18.4, 4.0 Hz), −108.06 (d, J=56.1 Hz).

### Example #274: Enantiomer (10S) or (10R) of N-[4-(difluoromethyl)phenyl]-2-[3-fluoro-5-(3-fluoroazetidin-1-yl)-10-methyl-9-oxo-4,8,12-triazatricyclo[9.4.0.0^{2,7}]pentadeca-1(15),2,4,6,11,13-hexaen-8-yl]acetamide

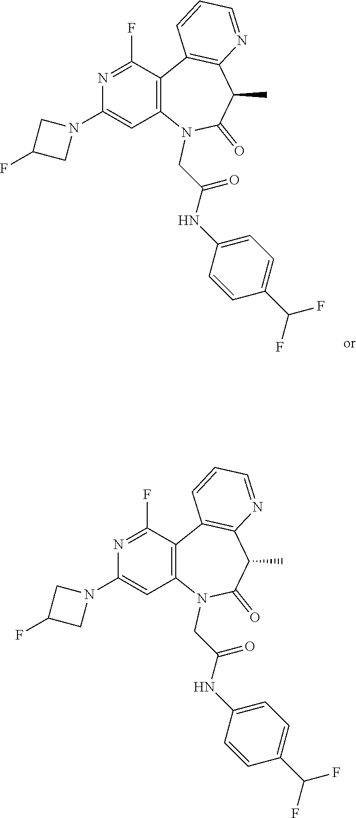 [SMILES: C[C@H]1C(=O)N(CC(=O)Nc2ccc(C(F)F)cc2)c2cc(N3CC(F)C3)nc(F)c2-c2cccnc21 ; C[C@@H]1C(=O)N(CC(=O)Nc2ccc(C(F)F)cc2)c2cc(N3CC(F)C3)nc(F)c2-c2cccnc21]

The title product was prepared according to general procedure SnAr_B using 2-(3,5-difluoro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide NN230 and 3-fluoroazetidine hydrochloride. The residue was purified by preparative HPLC (Waters XBridge OBD MS C18 column using NH₄HCO₃ 10 mM+50 μL/L NH₄OH in water/MeCN (0 to 100%) as eluent) then SFC chiral chromatography (Reprosil Chiral NR-R, CO₂₊₂₀% IPA) to give the title compound (16 mg, yield: 14%) as a white solid and the other enantiomer as white solid (15 mg, Yield=13%). LC-MS m/z [M+H]⁺: 500; purity: 99%. ¹H NMR (400 MHz, DMSO-d₆) δ 10.43 (s, 1H), 8.58 (dd, J=4.8, 1.6 Hz, 1H), 8.03-7.97 (m, 1H), 7.67 (d, J=8.3 Hz, 2H), 7.51 (d, J=8.3 Hz, 2H), 7.44 (dd, J=7.9, 4.7 Hz, 1H), 6.96 (t, J=56.1 Hz, 1H), 6.41 (s, 1H), 5.62-5.40 (m, 1H), 4.65-4.49 (m, 2H), 4.36 (tdd, J=21.6, 10.7, 5.8 Hz, 2H), 4.18-4.00 (m, 2H), 3.79 (q, J=6.5 Hz, 1H), 1.47 (d, J=6.6 Hz, 3H). Chiral purity: 100%; rt=3.99 min (first eluting enantiomer) column SFC ReproSil-Chiral-NR-R, CO₂; 20% IPA+0.1% DEA.

### Example #275: Enantiomer (10S) or (10R) of N-[4-(difluoromethyl)phenyl]-2-[3-fluoro-5-(3-hydroxy-3-methyl-azetidin-1-yl)-10-methyl-9-oxo-4,8,12-triazatricyclo[9.4.0.0^{2,7}]pentadeca-1(15),2,4,6,11,13-hexaen-8-yl]acetamide

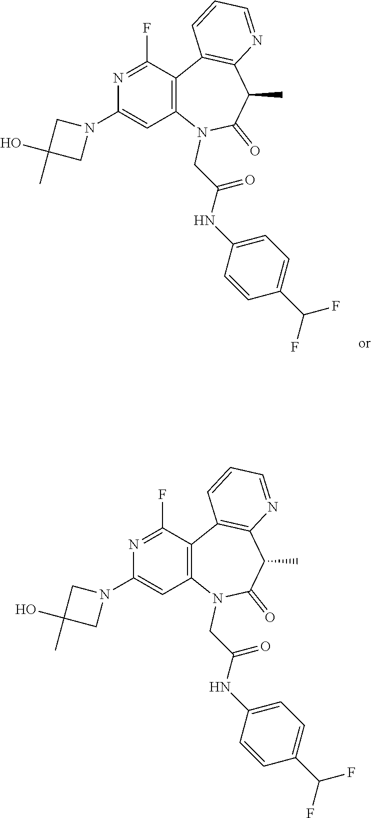 [SMILES: C[C@H]1C(=O)N(CC(=O)Nc2ccc(C(F)F)cc2)c2cc(N3CC(C)(O)C3)nc(F)c2-c2cccnc21 ; C[C@@H]1C(=O)N(CC(=O)Nc2ccc(C(F)F)cc2)c2cc(N3CC(C)(O)C3)nc(F)c2-c2cccnc21]

The title product was prepared according to general procedure SnAr_B using 2-(3,5-difluoro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide NN230 and 3-methylazetidin-3-ol hydrochloride. The residue was purified by preparative HPLC (Waters XBridge OBD MS C18 column using NH₄HCO₃ 10 mM+50 μl/L NH₄OH in water/MeCN (0 to 100%) as eluent) then SFC chiral chromatography (Reprosil Chiral NR-R, CO₂₊₂₀% EtOH) to give the title compound (15 mg, yield: 13%) as a white solid and the other enantiomer as white solid (31 mg, Yield=27%). LC-MS m/z [M+H]⁺: 512; purity: 99%. ¹H NMR (400 MHz, DMSO-d₆) δ 10.43 (s, 1H), 8.58 (dd, J=4.8, 1.5 Hz, 1H), 8.11-7.90 (m, 1H), 7.67 (d, J=8.3 Hz, 2H), 7.51 (d, J=8.3 Hz, 2H), 7.44 (dd, J=7.9, 4.7 Hz, 1H), 6.96 (t, J=56.1 Hz, 1H), 6.41 (s, 1H), 5.69-5.35 (m, 1H), 4.71-4.47 (m, 3H), 4.36 (tdd, J=21.6, 10.6, 5.9 Hz, 2H), 4.22-3.95 (m, 2H), 3.79 (q, J=6.5 Hz, 1H), 1.47 (d, J=6.6 Hz, 3H). Two hydrogens are under solvent peaks. Chiral purity: 100%; rt=2.62 min (first eluting enantiomer) column SFC ReproSil-Chiral-NR-R, CO₂₊₂₀% EtOH+0.1% DEA. For information, second eluting enantiomer rt=5.14 min (100%).

### Example #276 and #277: Enantiomer (10S,3S) or (10S,3R) or (10R,3S) or (10R,3R)) of N-[4-(difluoromethyl)phenyl]-2-[3-fluoro-5-[3-fluoropyrrolidin-1-yl]-10-methyl-9-oxo-4,8,12-triazatricyclo[9.4.0.0^{2,7}]pentadeca-1(15),2,4,6,11,13-hexaen-8-yl]acetamide

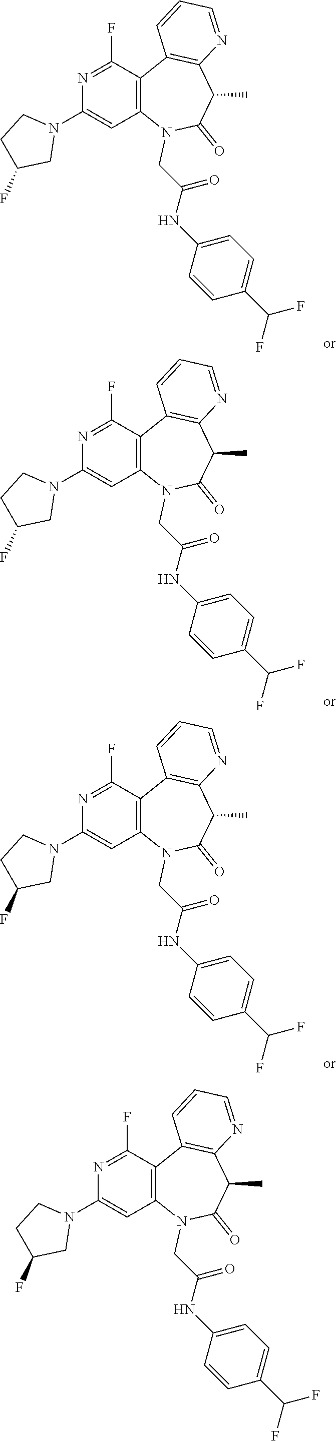

The title product was prepared according to general procedure SnAr_D using 2-(3,5-difluoro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide NN230 and 3-fluoropyrrolidine hydrochloride (211.9 mg, 1.69 mmol). The residue was purified by preparative HPLC (SFAR_C18 cartridge cartridge using NH₄HCO₃ 0.01M and NH₄OH 0.001M in water/MeCN (5 to 95%) as eluent). The chiral separation of the four diastereoisomers was done by a first SFC chiral chromatography (Reprosil Chiral NR-R, CO₂₊₂₀% EtOH) that gave two fractions from which the first eluting fraction (F1) was further separated by a second SFC chiral chromatography (AD, CO₂₊₃₀% EtOH) to afford the first diastereoisomer #276 (40 mg, yield: 14%) as a white solid. LC-MS (basic method B2) m/z [M+H]⁺: 514; rt: 4.37 min; purity: 99%. LC-MS (method A2) m/z [M+H]⁺: 514; rt: 4.61 min; purity: 99%. ¹H NMR (400 MHz, DMSO-d₆) δ 10.44 (s, 1H), 8.57 (dd, J=4.8, 1.7 Hz, 1H), 8.00 (ddd, J=8.0, 4.4, 1.7 Hz, 1H), 7.68 (d, J=8.4 Hz, 2H), 7.51 (d, J=8.3 Hz, 2H), 7.44 (dd, J=7.9, 4.7 Hz, 1H), 6.96 (t, J=56.1 Hz, 1H), 6.49 (s, 1H), 5.46 (d, J=53.1 Hz, 1H), 4.82-4.45 (m, 2H), 3.81 (q, J=6.6 Hz, 1H), 3.73 (s, 1H), 3.68-3.56 (m, 2H), 3.50 (q, J=10.2 Hz, 1H), 2.36-2.21 (m, 2H), 1.47 (d, J=6.6 Hz, 3H). Chiral purity: 100%; rt=2.71 min (first eluting diastereoisomer) column SFC Chiral-NR-R, CO₂₊₃₀% EtOH+0.1% DEA. The second diastereoisomer #277 was also obtained from F1, 35 mg, Yield=12%): LC-MS m/z [M+H]⁺: 514; rt: 4.37 min; purity: 99%. LC-MS m/z [M+H]⁺: 514; rt: 4.61 min; purity: 99% (second eluting enantiomer) rt=2.79 min (73%)

### Example #278: Enantiomer (10S) or (10R) of N-[4-(difluoromethyl)phenyl]-2-[5-(3,3-difluoropyrrolidin-1-yl)-3-fluoro-10-methyl-9-oxo-4,8,12-triazatricyclo[9.4.0.0^{2,7}]pentadeca-1(15),2,4,6,11,13-hexaen-8-yl]acetamide

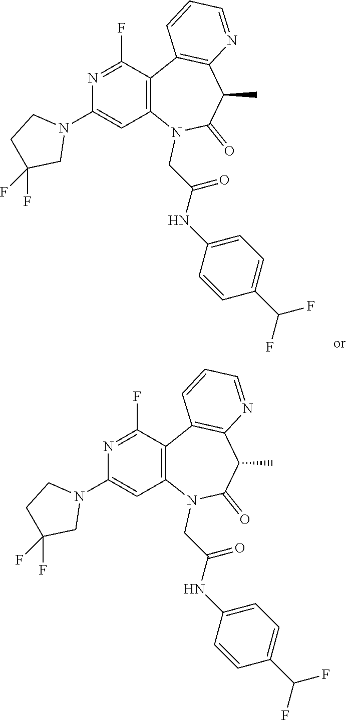

The title product was prepared according to general procedure SnAr_D using 2-(3,5-difluoro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide NN230 and 3,3-difluoropiperidine hydrochloride. The residue was purified by preparative HPLC (SFAR_C18 60 g cartridge using NH₄HCO₃ 0.01M and NH₄OH 0.001M NH₄OH in water/MeCN (5 to 95%) as eluent) then SFC chiral chromatography (Reprosil Chiral NR-R, CO₂₊₂₀% MeOH) to give the title compound (58 mg, yield: 19%) as a white solid and the other enantiomer as white solid (60 mg, Yield=20%). LC-MS m/z [M+H]⁺: 532; purity: 100%. ¹H NMR (400 MHz, DMSO-d₆) δ 10.43 (s, 1H), 8.58 (d, J=4.5 Hz, 1H), 8.01 (s, 1H), 7.67 (d, J=8.2 Hz, 2H), 7.51 (d, J=8.3 Hz, 2H), 7.44 (dd, J=8.4, 4.4 Hz, 1H), 7.15-6.79 (m, 1H), 6.52 (s, 1H), 4.62 (q, J=16.6 Hz, 2H), 4.04-3.57 (m, 6H), 2.58 (d, J=7.4 Hz, 1H), 1.47 (d, J=6.5 Hz, 3H). Chiral purity: 100%; rt=2.4 min (first eluting enantiomer) column HPLC ReproSil-Chiral-NR-R with EtOH/Heptane 1:1+0.1% DEA as eluent. For information, second eluting enantiomer rt=2.98 min (100%).

### Example #279 and #285: Enantiomer (10S,3S) or (10S,3R) or (10R,3S) or (10R,3R) of N-[4-(difluoromethyl)phenyl]-2-[3-fluoro-5-[3-hydroxypyrrolidin-1-yl]-10-methyl-9-oxo-4,8,12-triazatricyclo[9.4.0.0^{2,7}]pentadeca-1(15),2,4,6,11,13-hexaen-8-yl]acetamide

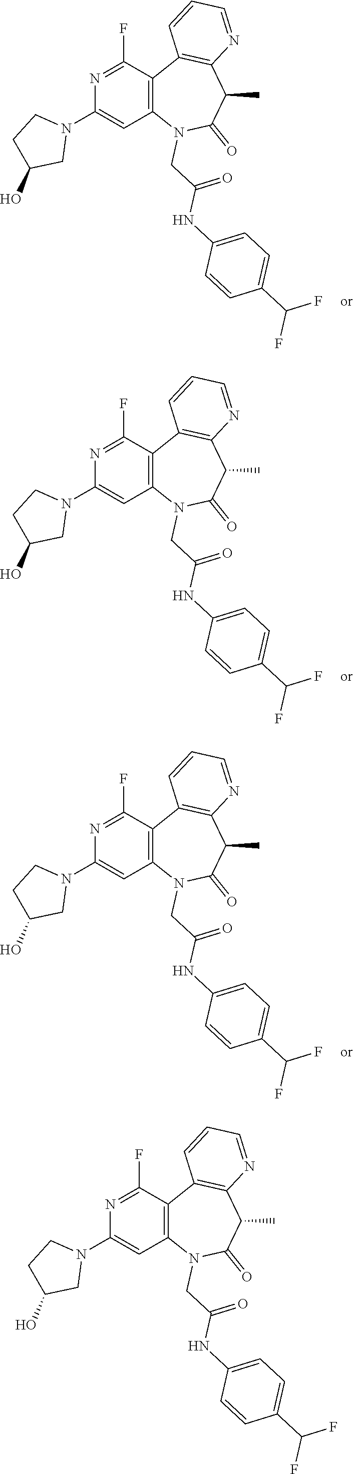 [SMILES: C[C@H]1C(=O)N(CC(=O)Nc2ccc(C(F)F)cc2)c2cc(N3CC[C@H](O)C3)nc(F)c2-c2cccnc21 ; C[C@@H]1C(=O)N(CC(=O)Nc2ccc(C(F)F)cc2)c2cc(N3CC[C@H](O)C3)nc(F)c2-c2cccnc21 ; C[C@H]1C(=O)N(CC(=O)Nc2ccc(C(F)F)cc2)c2cc(N3CC[C@@H](O)C3)nc(F)c2-c2cccnc21 ; C[C@@H]1C(=O)N(CC(=O)Nc2ccc(C(F)F)cc2)c2cc(N3CC[C@@H](O)C3)nc(F)c2-c2cccnc21]

The title product was prepared according to general procedure SnAr_D using 2-(3,5-difluoro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide NN230 and 3-pyrrolidinol. The residue was purified first by preparative HPLC (SFAR_C18 60 g cartridge using NH₄HCO₃ 0.01M and NH₄OH 0.001M NH₄OH in water/MeCN (5 to 95%) as eluent) to afford the racemic compound (99 mg, Yield 27%) as a white solid. LC-MS m/z [M+H]⁺: 512; purity: 95.5%. The chiral separation of the four diastereoisomers was done by a first SFC chiral chromatography (Reprosil Chiral NR-R, CO₂₊₂₀% MeOH) that gave two fractions from which the first eluting fraction F1 was further separated by liquid chiral chromatography (Chiralpak IG from Daicel using Heptane-IPA 1:1 as solvents) to give the title compound (the first diastereoisomer #279, first eluting) (8.3 mg, yield: 2.4%) as a white solid and the second diastereoisomer #285 (second eluting) as a white solid (10.3 mg, Yield=3%). LC-MS (method B2) m/z [M+H]⁺: 512; rt: 3.84 min; purity: 99%. LC-MS (method A2) m/z [M+H]⁺: 512; rt: 3.75 min; purity: 87%. ¹H NMR (400 MHz, DMSO-d₆) δ 10.44 (s, 1H), 8.55 (dd, J=4.7, 1.7 Hz, 1H), 8.05-7.91 (m, 1H), 7.68 (d, J=8.3 Hz, 2H), 7.51 (d, J=8.4 Hz, 2H), 7.43 (dd, J=7.9, 4.8 Hz, 1H), 6.96 (t, J=56.1 Hz, 1H), 6.41 (s, 1H), 5.04 (d, J=3.4 Hz, 1H), 4.58 (s, 2H), 4.39 (s, 1H), 3.80 (q, J=6.5 Hz, 1H), 3.52 (dt, J=11.2, 5.8 Hz, 1H), 2.09-1.97 (m, 1H), 1.91 (s, 1H), 1.47 (d, J=6.6 Hz, 3H), 1.23 (s, 1H). Two hydrogens are under solvent peaks. Chiral purity: 99.8%; rt=2.17 min (first eluting enantiomer) column HPLC ChiralPak IG-u with EtOH/Heptane 1:1+0.1% DEA as eluent. #285: LC-MS (basic) m/z [M+H]⁺: 512; rt: 3.83 min; purity: 98%. LC-MS (acid) m/z [M+H]⁺: 512; rt: 3.74 min; purity: 88%, second eluting enantiomer rt=2.74 min (100%).

### Example #280: Enantiomer (10S) or (10R) of 2-[5-(3,3-difluoroazetidin-1-yl)-3-fluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide

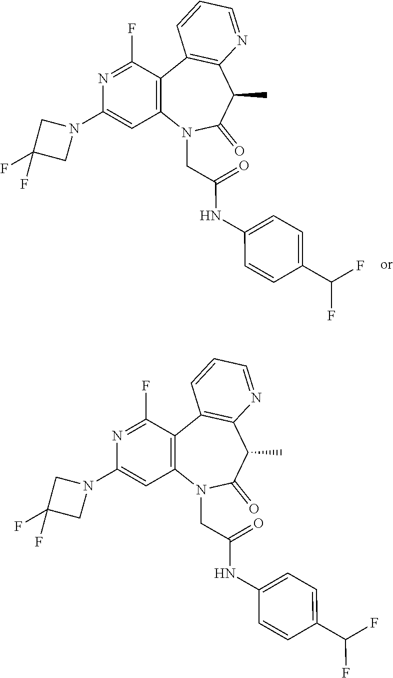 [SMILES: C[C@H]1C(=O)N(CC(=O)Nc2ccc(C(F)F)cc2)c2cc(N3CC(F)(F)C3)nc(F)c2-c2cccnc21 ; C[C@@H]1C(=O)N(CC(=O)Nc2ccc(C(F)F)cc2)c2cc(N3CC(F)(F)C3)nc(F)c2-c2cccnc21]

The title product was prepared according to general procedure SnAr_B using 2-[(10R)-3,5-difluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide NN230a and 3,3-difluoroazetidine hydrochloride. The residue was purified by preparative HPLC (Waters XBridge OBD MS C18 column using NH₄HCO₃ 10 mM+50 μl/L NH₄OH in water/MeCN (0 to 100%) as eluent) then SFC chromatography (column Silica, CO₂ using EtOH as solvent from 5% to 50%) to give the title compound (36.4 mg, yield: 22%) as a white solid. LC-MS m/z [M+H]⁺: 518; purity: 100%. ¹H NMR (400 MHz, DMSO-d₆) δ 10.44 (s, 1H), 8.60 (dd, J=4.7, 2.0 Hz, 1H), 8.07-7.99 (m, 1H), 7.67 (d, J=8.2 Hz, 2H), 7.51 (d, J=8.3 Hz, 2H), 7.47-7.41 (m, 1H), 6.96 (t, J=56.1 Hz, 1H), 6.55 (s, 1H), 4.67-4.38 (m, 6H), 3.81 (q, J=6.5 Hz, 1H), 1.48 (d, J=6.6 Hz, 3H). The chirality was measured by chiral UHPLC (Reprosil CHIRAL NR-R, EtOH 50%-Heptane 50%-DEA 0.1%, T 30° c.; RT 2.7 min; purity: 100%).

### Example #281: Enantiomer (10S) or (10R) of 2-[5-(6,6-difluoro-2-azaspiro[3.3]heptan-2-yl)-3-fluoro-10-methyl-9-oxo-4,8,12-triazatricyclo[9.4.0.0^{2,7}]pentadeca-1(15),2,4,6,11,13-hexaen-8-yl]-N-[4-(difluoromethyl)phenyl]acetamide

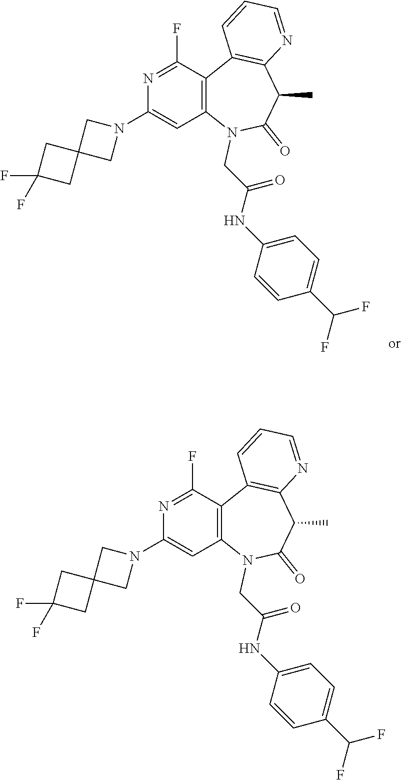 [SMILES: C[C@H]1C(=O)N(CC(=O)Nc2ccc(C(F)F)cc2)c2cc(N3CC4(C3)CC(F)(F)C4)nc(F)c2-c2cccnc21 ; C[C@@H]1C(=O)N(CC(=O)Nc2ccc(C(F)F)cc2)c2cc(N3CC4(C3)CC(F)(F)C4)nc(F)c2-c2cccnc21]

The title product was prepared according to general procedure SnAr_B using 2-[(10R)-3,5-difluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide NN230a and 6,6-difluoro-2-azaspiro[3.3]heptane trifluoroacetate. The crude product was purified by preparative HPLC (Purification Method P_A) to afford the title compound (9.7 mg, yield: 26%) as a white solid. LC-MS m/z: [M+H]⁺: 558.2; purity: 97%.

### Example #282: Enantiomer (10S) or (10R) of N-[4-(difluoromethyl)phenyl]-2-[3-fluoro-10-methyl-5-(6-oxa-2-azaspiro[3.4]octan-2-yl)-9-oxo-4,8,12-triazatricyclo[9.4.0.0^{2,7}]pentadeca-1(15),2,4,6,11,13-hexaen-8-yl]acetamide

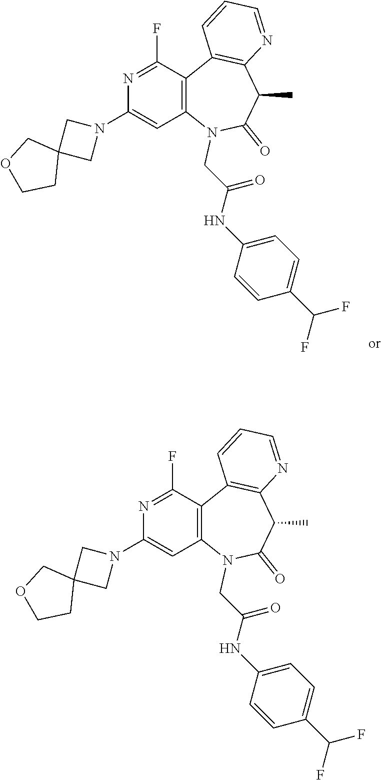 [SMILES: C[C@H]1C(=O)N(CC(=O)Nc2ccc(C(F)F)cc2)c2cc(N3CC4(CCOC4)C3)nc(F)c2-c2cccnc21 ; C[C@@H]1C(=O)N(CC(=O)Nc2ccc(C(F)F)cc2)c2cc(N3CC4(CCOC4)C3)nc(F)c2-c2cccnc21]

The title product was prepared according to general procedure SnAr_B using 2-[(10R)-3,5-difluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide NN230a and 6-oxa-2-aza-spiro[3.4]octane oxalate (28.0 mg, 0.14 mmol). The crude product was purified by preparative HPLC (Purification Method P_A) to afford the title compound (22.7 mg, yield: 63%) as a white solid. LC-MS m/z: [M+H]⁺: 538.2; purity: 98%.

### Example #283: Enantiomer (10S) or (10R) of 2-[5-(5-azaspiro[2.3]hexan-5-yl)-3-fluoro-10-methyl-9-oxo-4,8,12-triazatricyclo[9.4.0.0^{2,7}]pentadeca-1(15),2,4,6,11,13-hexaen-8-yl]-N-[4-(difluoromethyl)phenyl]acetamide

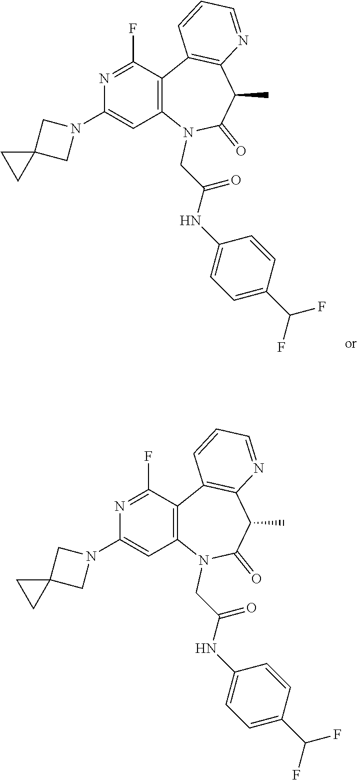 [SMILES: C[C@H]1C(=O)N(CC(=O)Nc2ccc(C(F)F)cc2)c2cc(N3CC4(CC4)C3)nc(F)c2-c2cccnc21 ; C[C@@H]1C(=O)N(CC(=O)Nc2ccc(C(F)F)cc2)c2cc(N3CC4(CC4)C3)nc(F)c2-c2cccnc21]

The title product was prepared according to general procedure SnAr_B using 2-[(10R)-3,5-difluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide NN230a and 5-azaspiro[2.3]hexane ethanedioate. The residue was purified by SFC chromatography (GreenSep Nitro 5p 120A, CO₂ using MeOH as solvent from 5% to 50%) to give the title compound (74 mg, yield: 43%) as a white solid. LC-MS m/z [M+H]⁺: 508; purity: 100%. ¹H NMR (400 MHz, DMSO-d₆) δ 10.45 (s, 1H), 8.57 (d, J=4.7 Hz, 1H), 7.99 (t, J=6.2 Hz, 1H), 7.68 (d, J=8.2 Hz, 2H), 7.51 (d, J=8.3 Hz, 2H), 7.43 (dd, J=7.9, 4.7 Hz, 1H), 6.96 (t, J=56.1 Hz, 1H), 6.35 (s, 1H), 4.56 (s, 2H), 4.18-4.01 (m, 4H), 3.81 (q, J=6.6 Hz, 1H), 1.47 (d, J=6.6 Hz, 3H), 0.76-0.60 (m, 4H).

### Example #284: Enantiomer (10S) or (10R) of N-[4-(difluoromethyl)phenyl]-2-[3-fluoro-10-methyl-5-(2-oxa-6-azaspiro[3.3]heptan-6-yl)-9-oxo-4,8,12-triazatricyclo[9.4.0.0^{2,7}]pentadeca-1(15),2,4,6,11,13-hexaen-8-yl]acetamide

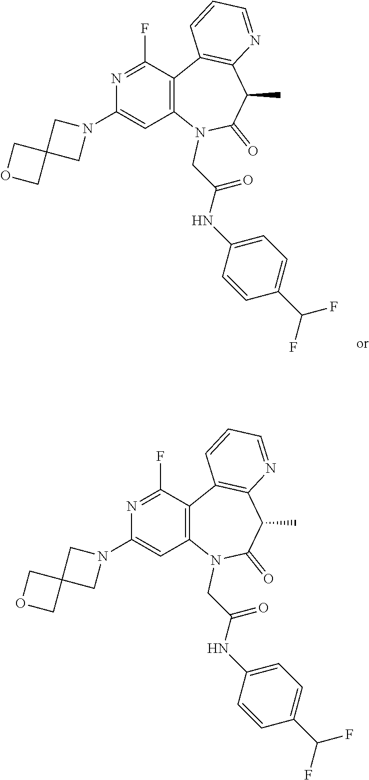 [SMILES: C[C@H]1C(=O)N(CC(=O)Nc2ccc(C(F)F)cc2)c2cc(N3CC4(COC4)C3)nc(F)c2-c2cccnc21 ; C[C@@H]1C(=O)N(CC(=O)Nc2ccc(C(F)F)cc2)c2cc(N3CC4(COC4)C3)nc(F)c2-c2cccnc21]

The title product was prepared according to general procedure SnAr_B using 2-[(10R)-3,5-difluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide NN230a and 2-oxa-6-azaspiro[3.3]heptane hemioxalate (38.9 mg, 0.14 mmol). The residue was purified by preparative HPLC (Purification Method P_A) to afford the title compound (18.8 mg, yield: 53%) as a white solid. LC-MS m/z: [M+H]⁺: 524.2; purity: 98%.

### Example #286: Enantiomer (10S) or (10R) of 2-[5-(2,2-difluoro-5-azaspiro[2.3]hexan-5-yl)-3-fluoro-10-methyl-9-oxo-4,8,12-triazatricyclo[9.4.0.0^{2,7}]pentadeca-1(15),2,4,6,11,13-hexaen-8-yl]-N-[4-(difluoromethyl)phenyl]acetamide

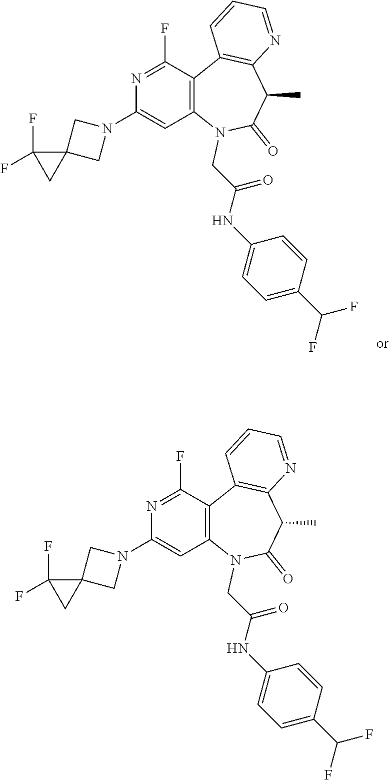 [SMILES: C[C@H]1C(=O)N(CC(=O)Nc2ccc(C(F)F)cc2)c2cc(N3CC4(C3)CC4(F)F)nc(F)c2-c2cccnc21 ; C[C@@H]1C(=O)N(CC(=O)Nc2ccc(C(F)F)cc2)c2cc(N3CC4(C3)CC4(F)F)nc(F)c2-c2cccnc21]

The title product was prepared according to general procedure SnAr_B using 2-[(10R)-3,5-difluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide NN230a and 1,1-difluoro-5-azaspiro[2.3]hexane. The residue was purified by SFC chromatography (GreenSep Nitro 5μ 120A, CO₂ using EtOH as solvent from 5% to 50%) to give the title compound (114 mg, yield: 47%) as a white solid. LC-MS m/z [M+H]⁺: 544; purity: 100%. ¹H NMR (400 MHz, DMSO-d₆) δ 10.43 (s, 1H), 8.58 (dd, J=4.7, 1.7 Hz, 1H), 8.01 (ddd, J=7.9, 4.5, 1.7 Hz, 1H), 7.67 (d, J=8.4 Hz, 2H), 7.54-7.48 (m, 2H), 7.45 (dd, J=7.9, 4.8 Hz, 1H), 6.96 (t, J=56.1 Hz, 1H), 6.46 (s, 1H), 4.67-4.49 (m, 2H), 4.27-4.09 (m, 4H), 3.81 (q, J=6.6 Hz, 1H), 1.80 (ft, J=13.1, 6.5 Hz, 2H), 1.47 (d, J=6.6 Hz, 3H). The chirality was measured by chiral UHPLC (Reprosil CHIRAL NR-R, EtOH 50%-Heptane 50%-DEA 0.1%, T 30° c.; RT 2.4 min; purity: 100%).

### Example #287: Enantiomer (10S) or (10R) of 2-[5-(6-azabicyclo[3.1.1]heptan-6-yl)-3-fluoro-10-methyl-9-oxo-4,8,12-triazatricyclo[9.4.0.0^{2,7}]pentadeca-1(15),2,4,6,11,13-hexaen-8-yl]-N-[4-(difluoromethyl)phenyl]acetamide

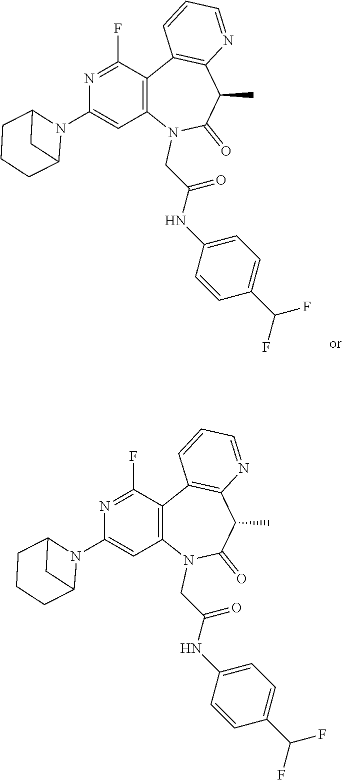 [SMILES: C[C@H]1C(=O)N(CC(=O)Nc2ccc(C(F)F)cc2)c2cc(N3C4CCCC3C4)nc(F)c2-c2cccnc21 ; C[C@@H]1C(=O)N(CC(=O)Nc2ccc(C(F)F)cc2)c2cc(N3C4CCCC3C4)nc(F)c2-c2cccnc21]

The title product was prepared according to general procedure SnAr_B using 2-[(10R)-3,5-difluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide NN230a and azabicyclo[3.1.1]heptane hydrochloride. The residue was purified by preparative HPLC (Waters XBridge OBD MS C18 column using NH₄HCO₃ 10 mM+50 μl/L NH₄OH in water/MeCN (0 to 100%) as eluent) to give the title compound (39 mg, yield: 66%) as a white solid. LC-MS m/z [M+H]⁺: 522; purity: 99%. ¹H NMR (400 MHz, DMSO-d₆) δ 10.42 (s, 1H), 8.57 (dd, J=4.8, 1.7 Hz, 1H), 8.00 (ddd, J=7.9, 4.6, 1.7 Hz, 1H), 7.65 (d, J=8.4 Hz, 2H), 7.50 (d, J=8.1 Hz, 2H), 7.43 (dd, J=7.9, 4.7 Hz, 1H), 6.96 (t, J=56.1 Hz, 1H), 6.36 (s, 1H), 4.66-4.51 (m, 2H), 4.31 (td, J=5.5, 2.7 Hz, 2H), 3.81 (q, J=6.6 Hz, 1H), 2.23 (dq, J=14.3, 7.3 Hz, 2H), 1.85 (dq, J=15.1, 7.7 Hz, 1H), 1.73-1.61 (m, 3H), 1.48 (d, J=6.6 Hz, 3H), 1.37 (s, 1H). One hydrogen under solvent peak.

### Example #288: Enantiomer (10S) or (10R) of N-[4-(difluoromethyl)phenyl]-2-[3-fluoro-5-(3-fluoro-3-methyl-azetidin-1-yl)-10-methyl-9-oxo-4,8,12-triazatricyclo[9.4.0.0^{2,7}]pentadeca-1(15),2,4,6,11,13-hexaen-8-yl]acetamide

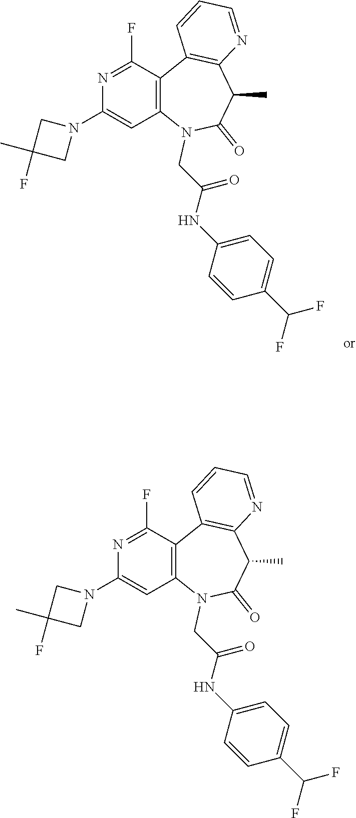 [SMILES: C[C@H]1C(=O)N(CC(=O)Nc2ccc(C(F)F)cc2)c2cc(N3CC(C)(F)C3)nc(F)c2-c2cccnc21 ; C[C@@H]1C(=O)N(CC(=O)Nc2ccc(C(F)F)cc2)c2cc(N3CC(C)(F)C3)nc(F)c2-c2cccnc21]

The title product was prepared according to general procedure SnAr_B using 2-[(10R)-3,5-difluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide NN230a and 3-fluoro-3-methylazetidine hydrochloride. The residue was purified first by preparative HPLC (Waters XBridge OBD MS C18 column using NH₄HCO₃ 10 mM+50 μl/L NH₄OH in water/MeCN (0 to 100%) as eluent) and then by SFC chromatography (GreenSep Nitro 5p 120A, CO₂ using MeOH as solvent from 5% to 50%) to give the title compound (6 mg, yield: 10%) as a white solid. LC-MS m/z [M+H]⁺: 514; purity: 97%.

### Example #289: Enantiomer (10S) or (10R) of 2-[5-(6,6-difluoro-3-azabicyclo[3.1.0]hexan-3-yl)-3-fluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide

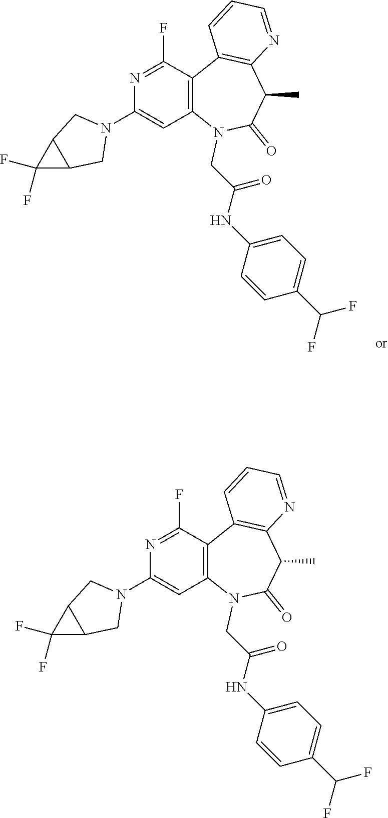 [SMILES: C[C@H]1C(=O)N(CC(=O)Nc2ccc(C(F)F)cc2)c2cc(N3CC4C(C3)C4(F)F)nc(F)c2-c2cccnc21 ; C[C@@H]1C(=O)N(CC(=O)Nc2ccc(C(F)F)cc2)c2cc(N3CC4C(C3)C4(F)F)nc(F)c2-c2cccnc21]

The title product was prepared according to general procedure SnAr_B using 2-[(10R)-3,5-difluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide NN230a and 6,6-difluoro-3-azabicyclo[3.1.0]hexane hydrochloride. The residue was purified first by preparative HPLC (Waters XBridge OBD MS C18 column using NH₄HCO₃ 10 mM+50 μl/L NH₄OH in water/MeCN (0 to 100%) as eluent) to give the title compound (40 mg, yield: 65%) as a white solid. LC-MS m/z [M+H]⁺: 544; purity: 99%. ¹H NMR (400 MHz, DMSO-d₆) δ 10.43 (s, 1H), 8.57 (dd, J=4.8, 1.7 Hz, 1H), 7.99 (ddd, J=7.9, 4.5, 1.7 Hz, 1H), 7.68 (d, J=8.4 Hz, 2H), 7.54-7.49 (m, 2H), 7.43 (dd, J=7.9, 4.8 Hz, 1H), 6.97 (t, J=56.1 Hz, 1H), 6.45 (s, 1H), 4.67-4.53 (m, 2H), 3.89-3.67 (m, 5H), 2.78-2.69 (m, 2H), 1.46 (d, J=6.6 Hz, 3H). The chirality was measured by chiral UHPLC (Whelk 0-1 (R,R) from Regis Technology, EtOH 50%-Heptane 50%-DEA 0.1%, T 30° c.; RT 3.36 min; purity: 95%).

### Example #290: Enantiomer (10S) or (10R) of 2-[5-(6,6-difluoro-3-azabicyclo[3.1.1]heptan-3-yl)-3-fluoro-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide

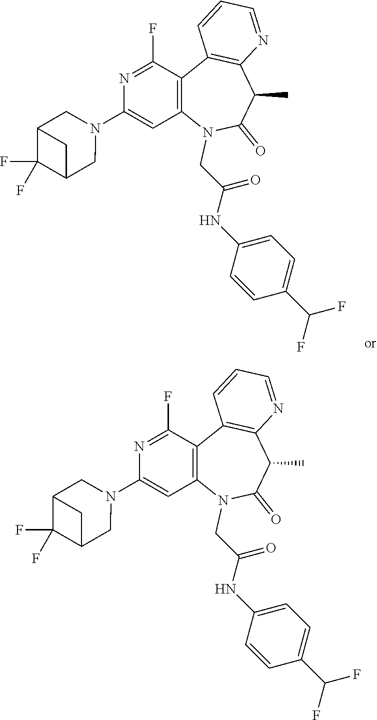

The title product was prepared according to general procedure SnAr_B using 2-[(10R)-3,5-difluoro-10-methyl-9-oxo-4,8,12-triazatricyclo[9.4.0.0^{2,7}]pentadeca-1(11),2(7).3,5,12,14-hexaen-8-yl]-N-[4-(difluoromethyl)phenyl]acetamide NN230a and 6,6-difluoro-3-azabicyclo[3.1.1]heptane hydrochloride. The residue was purified first by preparative HPLC (Waters XBridge OBD MS C18 column using NH₄HCO₃ 10 mM+50 μL/L NH₄OH in water/MeCN (0 to 100%) as eluent) to give the title compound (35 mg, yield: 56%) as a white solid. LC-MS m/z [M+H]⁺: 558; purity: 99%. ¹H NMR (400 MHz, DMSO-d₆) δ 10.44 (s, 1H), 8.58 (dd, J=4.7, 1.7 Hz, 1H), 8.01 (ddd, J=7.9, 4.4, 1.7 Hz, 1H), 7.68 (d, J=8.4 Hz, 2H), 7.51 (d, J=8.4 Hz, 2H), 7.45 (dd, J=7.9, 4.8 Hz, 1H), 6.96 (t, J=56.1 Hz, 1H), 6.61 (s, 1H), 4.69-4.55 (m, 2H), 4.00 (d, J=11.7 Hz, 1H), 3.91 (s, 1H), 3.82 (q, J=6.6 Hz, 1H), 3.71 (d, J=11.7 Hz, 1H), 3.63 (d, J=11.7 Hz, 1H), 3.08 (t, J=7.8 Hz, 2H), 2.06 (d, J=14.3 Hz, 1H), 1.58 (dd, J=17.6, 9.8 Hz, 1H), 1.47 (d, J=6.6 Hz, 3H). The chirality was measured by chiral UHPLC (Chiralpak IB from Daicel, EtOH 50%-Heptane 50%-DEA 0.1%, T 30° c.; RT 2.11 min; purity: 98%).

### Example #291: Enantiomer (7R) or (7S) of 2-[3-(3,3-difluoroazetidin-1-yl)-1,9-difluoro-7-methyl-6-oxo-7H-pyrido[4,3-d][3]benzazepin-5-yl]-N-[4-(difluoromethyl)phenyl]acetamide

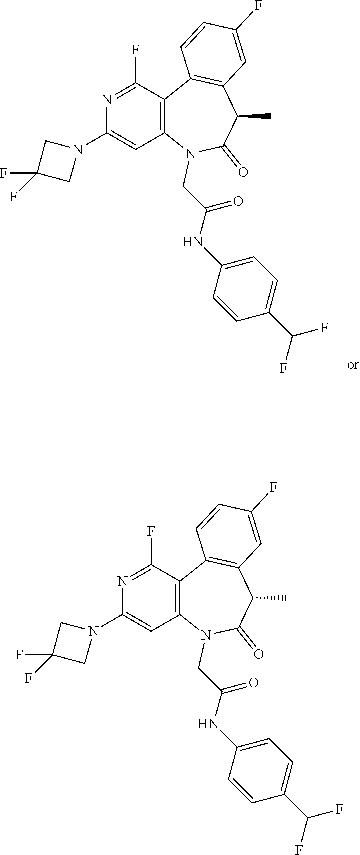 [SMILES: C[C@H]1C(=O)N(CC(=O)Nc2ccc(C(F)F)cc2)c2cc(N3CC(F)(F)C3)nc(F)c2-c2ccc(F)cc21 ; C[C@@H]1C(=O)N(CC(=O)Nc2ccc(C(F)F)cc2)c2cc(N3CC(F)(F)C3)nc(F)c2-c2ccc(F)cc21]

The title product was prepared according to general procedure SnAr_B using N-[4-(difluoromethyl)phenyl]-2-(1,3,9-trifluoro-7-methyl-6-oxo-7H-pyrido[4,3-d][3]benzazepin-5-yl)acetamide NN197 and 3,3-difluoroazetidine hydrochloride. The residue was purified first by preparative SFC chromatography (Daicel DCpak P4VP, CO₂ with iPrOH from 5% to 50% as eluent) and then liquid chiral chromatography (Chiralpak IA from Daicel using heptane/IPA 1:1 as solvent) to give the title compound (8 mg, yield: 8%) and the second enantiomer (9.5 mg, Yield: 9%) both as white solids. LC-MS m/z [M+H]⁺: 535; purity: 100%. Chiral purity: 100%; rt=1.56 min (first eluting enantiomer) column HPLC ChiralPak IA with Heptane/IPA 1:1+0.1% DEA as eluent. For information, second eluting enantiomer rt=2.26 min (100%). LC-MS (basic) m/z [M+H]⁺: 535; rt: 4.88 min; purity: 100%. LC-MS (acid) m/z [M+H]⁺: 535; rt: 5.14 min; purity: 100%, both measured by HPLC.

### Example #292: 2-[3-chloro-5-(3,3-difluoroazetidin-1-yl)-10-methyl-9-oxo-4,8,12-triazatricyclo[9.4.0.0^{2,7}]pentadeca-1(15),2,4,6,11,13-hexaen-8-yl]-N-[4-(difluoromethyl)phenyl]acetamide

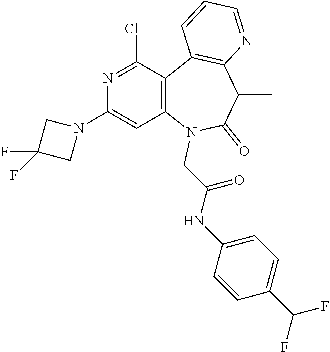 [SMILES: CC1C(=O)N(CC(=O)Nc2ccc(C(F)F)cc2)c2cc(N3CC(F)(F)C3)nc(Cl)c2-c2cccnc21]

The title product was prepared according to general procedure SnAr_D using 2-(3,5-dichloro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide (example #395) and 3,3-difluoroazetidine hydrochloride. The residue was purified by preparative HPLC (Waters XBridge OBD MS C18 column using NH₄HCO₃ 10 mM+50 μL/L NH₄OH in water/MeCN (0 to 100%) as eluent to afford a white solid (1.1 mg, Yield=4%). LC-MS m/z [M+H]⁺: 534; purity: 97%.

### Example #293: Enantiomer (10S) or (10R) of N-(4-chloro-3-fluoro-phenyl)-2-[5-(3,3-difluoroazetidin-1-yl)-10-methyl-9-oxo-4,8,12-triazatricyclo[9.4.0.0^{2,7}]pentadeca-1(15),2,4,6,11,13-hexaen-8-yl]acetamide

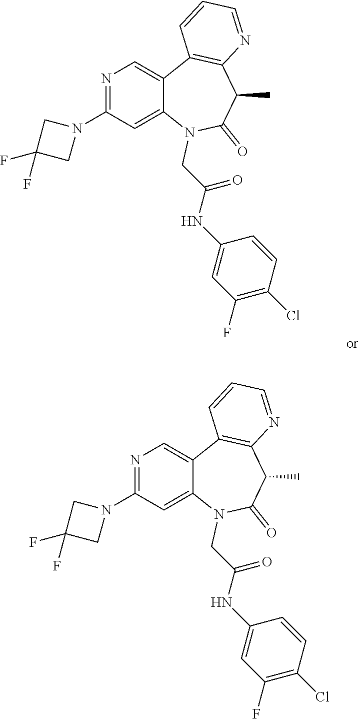 [SMILES: C[C@H]1C(=O)N(CC(=O)Nc2ccc(Cl)c(F)c2)c2cc(N3CC(F)(F)C3)ncc2-c2cccnc21 ; C[C@@H]1C(=O)N(CC(=O)Nc2ccc(Cl)c(F)c2)c2cc(N3CC(F)(F)C3)ncc2-c2cccnc21]

The title compound was prepared according to the general procedure 1 starting from 5-(3,3-difluoroazetidin-1-yl)-10-methyl-4,8,12-triazatricyclo[9.4.0.0^{2,7}]pentadeca-1 (11),2,4,6,12,14-hexaen-9-one NN180 (0.42 mmol) and 2-chloro-N-(4-chloro-3-fluoro-phenyl)acetamide (101.8 mg, 0.46 mmol). After purification by reverse phase HPLC (Purification Method P_B), the racemate was separated by Chiral HPLC (column CHIRALPAK IG, eluant EtOH-Heptane 1:1) to yield the pure enantiomer (36.2 mg, yield: 20%). LC-MS m/z [M+H]⁺: 502.1; purity: 100%. Chiral purity: 100%; rt=2.74 min (second eluting enantiomer). For information, first eluting enantiomer rt=1.80 min. Both measured by HPLC, Chiralpak IG, EtOH 50%—Heptane 50%—DEA 0.1% Example #294: 2-[5-(5-azaspiro[2.3]hexan-5-yl)-3-fluoro-9-oxo-4,8,12-triazatricyclo[9.4.0.0^{2,7}]pentadeca-1(15),2,4,6,11,13-hexaen-8-yl]-N-[4-(difluoromethyl)phenyl]acetamide

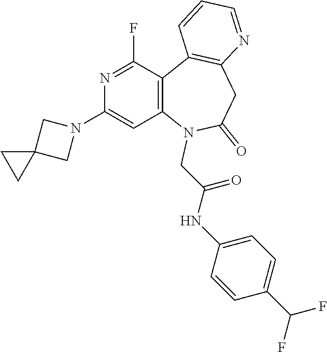 [SMILES: O=C(CN1C(=O)Cc2ncccc2-c2c1cc(N1CC3(CC3)C1)nc2F)Nc1ccc(C(F)F)cc1]

The title product was prepared according to general procedure SnAr_B using N-[4-(difluoromethyl)phenyl]-2-(3,5-difluoro-9-oxo-4,8,12-triazatricyclo[9.4.0.0^{2,7}]pentadeca-1(11),2(7),3,5,12,14-hexaen-8-yl)acetamide NN191 and 5-azaspiro[2.3]hexane oxalate. The reaction mixture was purified by preparative HPLC (using 0.3% NH₃ in water/MeCN 30 to 100% as eluent) to give the title compound (6.2 mg, yield: 44%) as a solid. LC-MS m/z [M+H]⁺: 494.2; purity: 90%.

### Example #295: 2-[5-(3,3-difluoroazetidin-1-yl)-3-fluoro-9-oxo-4,8,12-triazatricyclo[9.4.0.0^{2,7}]pentadeca-1(15),2,4,6,11,13-hexaen-8-yl]-N-[4-(difluoromethyl)phenyl]acetamide

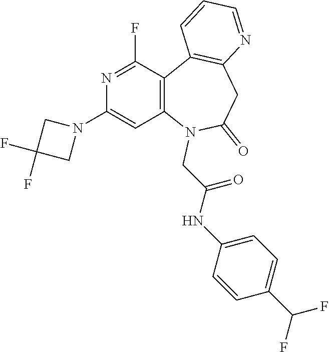 [SMILES: O=C(CN1C(=O)Cc2ncccc2-c2c1cc(N1CC(F)(F)C1)nc2F)Nc1ccc(C(F)F)cc1]

The title product was prepared according to general procedure SnAr_B using N-[4-(difluoromethyl)phenyl]-2-(3,5-difluoro-9-oxo-4,8,12-triazatricyclo[9.4.0.0^{2,7}]pentadeca-1(11),2(7),3,5,12,14-hexaen-8-yl)acetamide NN191 and 3,3-difluoroazetidine hydrochloride (43 mg, 0.315 mmol). The reaction mixture was purified by preparative HPLC (using 0.3% NH₃ in water/MeCN 30 to 100% as eluent) to give the title compound (3.4 mg, yield: 26%) as a white solid. LC-MS m/z [M+H]⁺: 504.1; purity: 99%

### Example #296: 2-[3-(3,3-difluoroazetidin-1-yl)-1,9-difluoro-6-oxo-7H-pyrido[4,3-d][3]benzazepin-5-yl]-N-[4-(difluoromethyl)phenyl]acetamide

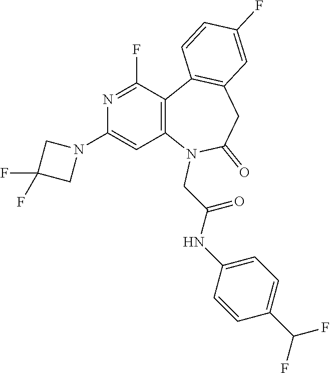 [SMILES: O=C(CN1C(=O)Cc2cc(F)ccc2-c2c1cc(N1CC(F)(F)C1)nc2F)Nc1ccc(C(F)F)cc1]

The title product was prepared according to general procedure SnAr_B using N-[4-(difluoromethyl)phenyl]-2-(3,5-difluoro-9-oxo-4,8,12-triazatricyclo[9.4.0.0^{2,7}]pentadeca-1(11),2(7),3,5,12,14-hexaen-8-yl)acetamide NN200 and 3,3-difluoroazetidine hydrochloride. The residue was purified by preparative SFC chromatography (Daicel DCpak P4VP, CO₂ with iPrOH from 5% to 50% as eluent) to give the title compound (40 mg, Yield: 35%) as a white solid. LC-MS (basic method B2) m/z [M+H]⁺: 521; rt: 4.738 min; purity: 100%. LC-MS m/z [M+H]⁺: 521; purity: 100%. ¹H NMR (400 MHz, DMSO-d₆) δ 10.44 (s, 1H), 7.69 (d, J=8.4 Hz, 2H), 7.66-7.62 (m, 1H), 7.52 (d, J=8.3 Hz, 2H), 7.37 (dd, J=9.3, 2.8 Hz, 1H), 7.26 (td, J=8.7, 2.8 Hz, 1H), 6.97 (t, J=56.1 Hz, 1H), 6.50 (s, 1H), 4.63-4.35 (m, 6H), 3.59 (s, 2H).

### Example #297: Enantiomer (10S) or (10R) of N-(4-chloro-3-fluoro-phenyl)-2-[3-methoxy-10-methyl-9-oxo-4,8,12-triazatricyclo[9.4.0.0^{2,7}]pentadeca-1(15),2(7),3,5,11,13-hexaen-8-yl]acetamide

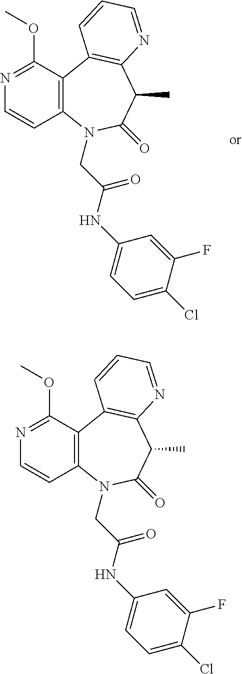 [SMILES: COc1nccc2c1-c1cccnc1[C@@H](C)C(=O)N2CC(=O)Nc1ccc(Cl)c(F)c1 ; COc1nccc2c1-c1cccnc1[C@H](C)C(=O)N2CC(=O)Nc1ccc(Cl)c(F)c1]

The title product was prepared according to the general procedure 1, starting from 3-methoxy-10-methyl-4,8,12-triazatricyclo[9.4.0.0^{2,7}]pentadeca-1(11),2(7),3,5,12,14-hexaen-9-one NN327 (100 mg, 0.38 mmol) and 2-chloro-N-(4-chloro-3-fluoro-phenyl)acetamide (CAS 895641-02-8). After purification by column chromatography on silica gel (using a gradient of 0-100% EtOAc in isohexane as eluent), the racemate was separated by chiral SFC (column Lux Amylose-1 from Phenomenex, eluent 50% MeOH, 50% CO₂) to yield the pure enantiomer as a white solid (11.4 mg, yield: 2%). LC-MS m/z [M+H]⁺: 441.2; purity: 96%. ¹H NMR (400 MHz, DMSO-d₆) δ 10.56 (s, 1H), 8.61 (dd, J=4.8, 1.7 Hz, 1H), 8.22 (d, J=5.8 Hz, 1H), 8.18 (dd, J=7.9, 1.8 Hz, 1H), 7.72 (dd, J=11.9, 2.4 Hz, 1H), 7.50 (t, J=8.7 Hz, 1H), 7.43 (dd, J=8.0, 4.7 Hz, 1H), 7.29 (dd, J=8.8, 2.4 Hz, 1H), 7.15 (d, J=5.8 Hz, 1H), 4.53 (dd, 2H), 3.92 (s, 3H), 3.65 (q, J=6.6 Hz, 1H), 1.48 (d, J=6.6 Hz, 3H). Chiral purity: 100%; rt=3.85 min (second eluting enantiomer). For information, first eluting enantiomer rt=1.1 min. Both measured by chiral SFC (column Lux 5 μM Amylose-1 from Phenomenex, eluent 50% MeOH+0.1% NH₃, 50% CO₂.

### Example #298: Enantiomer (10S) or (10R) of N-[4-(difluoromethyl)phenyl]-2-[3-methoxy-10-methyl-9-oxo-4,8,12-triazatricyclo[9.4.0.0^{2,7}]pentadeca-1(15),2(7),3,5,11,13-hexaen-8-yl]acetamide

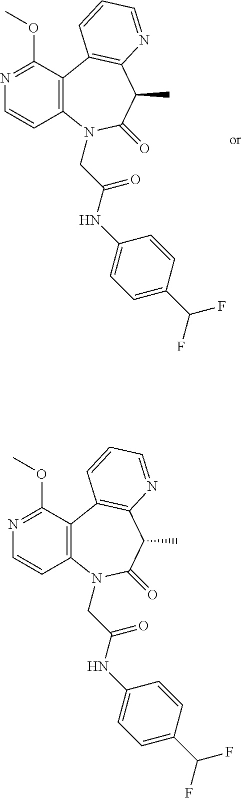 [SMILES: COc1nccc2c1-c1cccnc1[C@@H](C)C(=O)N2CC(=O)Nc1ccc(C(F)F)cc1 ; COc1nccc2c1-c1cccnc1[C@H](C)C(=O)N2CC(=O)Nc1ccc(C(F)F)cc1]

The title product was prepared according to the general procedure 1, starting from NN327 (100 mg, 0.38 mmol) and 2-chloro-N-[4-(difluoromethyl)phenyl]acetamide (CAS 872533-93-2). The racemate was separated by Chiral SFC (column Lux i-Cellulose-5, from Phenomenex, eluant 25% MeOH+0.03% NH₃ 75% CO₂) to yield the pure enantiomer as an off white solid (21 mg, yield: 15%). LC-MS m/z [M+H]⁺: 439.2; purity: 98%. ¹H NMR (400 MHz, DMSO-d₆) δ 10.46 (s, 1H), 8.61 (dd, J=4.7, 1.7 Hz, 1H), 8.23 (d, J=5.8 Hz, 1H), 8.18 (dd, J=8.0, 1.7 Hz, 1H), 7.68 (d, J=8.4 Hz, 2H), 7.51 (d, J=8.1 Hz, 2H), 7.44 (dd, J=7.9, 4.7 Hz, 1H), 7.16 (d, J=5.8 Hz, 1H), 6.96 (t, J=56.1 Hz, 1H), 4.55 (dd, 2H), 3.92 (s, 3H), 3.65 (q, J=6.6 Hz, 1H), 1.49 (d, J=6.6 Hz, 3H). Chiral purity: 98%; rt=2.31 min (second eluting enantiomer). For information, first eluting enantiomer rt=2.00 min. Both measured by SFC, CHIRALPAK IC from Daicel, 70% CO₂ and 30% MeOH+0.03% NH₃.

### Example #299: Enantiomer (10S) or (10R) of N-[4-(difluoromethyl)phenyl]-2-[5-methoxy-3,10-dimethyl-9-oxo-4,8,12-triazatricyclo[9.4.0.0^{2,7}]pentadeca-1(15),2(7),3,5,11,13-hexaen-8-yl]acetamide

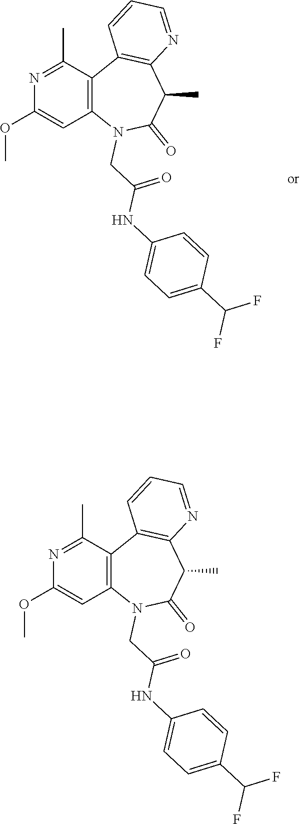 [SMILES: COc1cc2c(c(C)n1)-c1cccnc1[C@@H](C)C(=O)N2CC(=O)Nc1ccc(C(F)F)cc1 ; COc1cc2c(c(C)n1)-c1cccnc1[C@H](C)C(=O)N2CC(=O)Nc1ccc(C(F)F)cc1]

The title product was prepared according to the general procedure 1, starting from 5-methoxy-3,10-dimethyl-4,8,12-triazatricyclo[9.4.0.0²⁷]pentadeca-1(15),2(7).3,5,11,13-hexaen-9-one Intermediate NN328 (60%, 107 mg, 0.238 mmol) and 2-chloro-N-[4-(difluoromethyl)phenyl]acetamide (CAS 895641-02-8). After purification by column chromatography on silica gel (using a gradient of 0-100% EtOAc in isohexane as eluent), the racemate was separated by chiral SFC (column Lux i-Cellulose-5 from Phenomenex, eluent 35% MeOH, 65% CO₂) to yield the pure enantiomer (7.9 mg, yield: 4%). LC-MS m/z [M+H]⁺: 453.1; purity: 100%. ¹H NMR (400 MHz, DMSO-d₆) δ 10.39 (s, 1H), 8.59 (dd, J=4.8, 1.7 Hz, 1H), 8.04 (dd, J=7.9, 1.7 Hz, 1H), 7.67 (d, J=8.4 Hz, 2H), 7.51 (d, J=8.4 Hz, 2H), 7.44 (dd, J=7.8, 4.8 Hz, 1H), 6.96 (t, 1H, J=56 Hz), 6.78 (s, 1H), 4.52 (s, 2H), 3.91 (s, 3H), 3.76 (q, J=6.6 Hz, 1H), 2.47 (s, 3H), 1.47 (d, J=6.6 Hz, 3H). Chiral purity: 100%; rt=2.36 min (second eluting enantiomer). For information, first eluting enantiomer rt=1.79 min. Both measured by chiral SFC (column Lux i-Cellulose-5 from Phenomenex, eluent 35% MeOH+0.1% NH₃, 65% CO₂.

### Example #300: Enantiomer (10S) or (10R) of N-(4-chloro-3-fluoro-phenyl)-2-[5-methoxy-3,10-dimethyl-9-oxo-4,8,12-triazatricyclo[9.4.0.0^{2,7}]pentadeca-1(15),2(7),3,5,11,13-hexaen-8-yl]acetamide

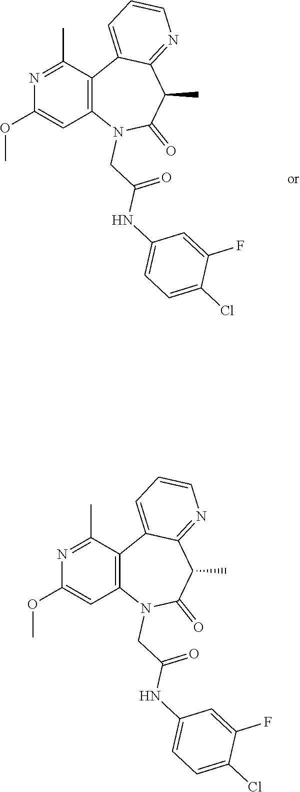 [SMILES: COc1cc2c(c(C)n1)-c1cccnc1[C@@H](C)C(=O)N2CC(=O)Nc1ccc(Cl)c(F)c1 ; COc1cc2c(c(C)n1)-c1cccnc1[C@H](C)C(=O)N2CC(=O)Nc1ccc(Cl)c(F)c1]

The title product was prepared according to the general procedure 1, starting from Intermediate NN328 (140 mg, 0.51 mmol) and 2-chloro-N-(4-chloro-3-fluoro-phenyl)acetamide (CAS 895641-02-8). The racemate was separated by Chiral SFC (column CHIRALPAK IC from Daicel, eluant 35% MeOH+0.1% NH₃, 65% CO₂) to yield the pure enantiomer as an off white solid (10 mg, yield: 6%). LC-MS m/z [M+H]⁺: 455.10, 457.10; purity: 98%. ¹H NMR (400 MHz, DMSO-d₆) δ 10.53 (s, 1H), 8.59 (dd, J=4.8, 1.6 Hz, 1H), 8.04 (dd, J=7.8, 1.7 Hz, 1H), 7.71 (dd, J=11.9, 2.4 Hz, 1H), 7.51 (t, J=8.7 Hz, 1H), 7.43 (dd, J=7.9, 4.7 Hz, 1H), 7.29 (ddd, J=8.8, 2.4, 1.0 Hz, 1H), 6.77 (s, 1H), 4.56-4.44 (m, 2H), 3.91 (s, 3H), 3.76 (q, J=6.6 Hz, 1H), 2.54 (s, 3H), 1.46 (d, J=6.6 Hz, 3H). Chiral purity: 99%; rt=2.46 min (First eluting enantiomer). For information, second eluting enantiomer rt=3.90 min. Both measured by SFC, CHIRALPAK IC from Daicel, 70% CO₂ and 30% MeOH+0.1% NH₃.

### Example #301: Enantiomer (10S) or (10R) of 2-[3-(difluoromethyl)-5,10-dimethyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide

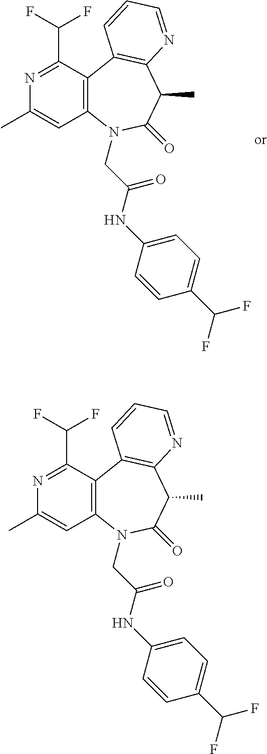 [SMILES: Cc1cc2c(c(C(F)F)n1)-c1cccnc1[C@@H](C)C(=O)N2CC(=O)Nc1ccc(C(F)F)cc1 ; Cc1cc2c(c(C(F)F)n1)-c1cccnc1[C@H](C)C(=O)N2CC(=O)Nc1ccc(C(F)F)cc1]

The title compound was prepared according to the general procedure 1 starting from 3-(difluoromethyl)-5,10-dimethyl-4,8,12-triazatricyclo[9.4.0.0^{2,7}]pentadeca-1(11),2(7),3,5,12,14-hexaen-9-one NN202 (273 mg, 0.94 mmol) and 2-chloro-N-[4-(difluoromethyl)phenyl]acetamide (248.7 mg, 1,132 mmol). The residue was purified by preparative basic reverse phase chromatography (YMC Triart—500 g−10 μm—76,5×200 mm using NH₄OH 0.13M in water/MeCN (5 to 85%) as eluent) and then by SFC chiral chromatography (Chiralpak IG from Daicel, CO₂ and 30% EtOH as eluent) to give the title compound (113.4 mg, yield: 25%) and the second enantiomer (113.7 mg, Yield: 25%) both as white solids. LC-MS (basic) m/z [M+H]⁺: 473; rt: 3.78 min; purity: 98%. LC-MS (acid) m/z [M+H]⁺: 473; rt: 4.21 min; purity: 98%. ¹H NMR (400 MHz, DMSO-d₆) δ 10.44 (s, 1H), 8.69 (d, J=4.7 Hz, 1H), 7.84 (d, J=7.8 Hz, 1H), 7.68 (d, J=8.3 Hz, 2H), 7.58 (s, 1H), 7.51 (d, J=7.4 Hz, 3H), 7.13-6.66 (m, 2H), 4.66-4.53 (m, 2H), 3.81 (q, J=6.6 Hz, 1H), 2.62 (s, 3H), 1.49 (d, J=6.6 Hz, 3H). Chiral purity: 100%; rt=2.93 min (second eluting enantiomer) column HPLC ChiralPak IG-u with Heptane/EtOH 1:1+0.1% DEA as eluent. For information, first eluting enantiomer rt=1.62 min (99%). LC-MS m/z [M+H]⁺: 473; purity: 97%.

### Example #302: Enantiomer (10S) or (10R) of N-(4-chloro-3-fluoro-phenyl)-2-[3-(difluoromethyl)-5,10-dimethyl-9-oxo-4,8,12-triazatricyclo[9.4.0.0^{2,7}]pentadeca-1(15),2,4,6,11,13-hexaen-8-yl]acetamide

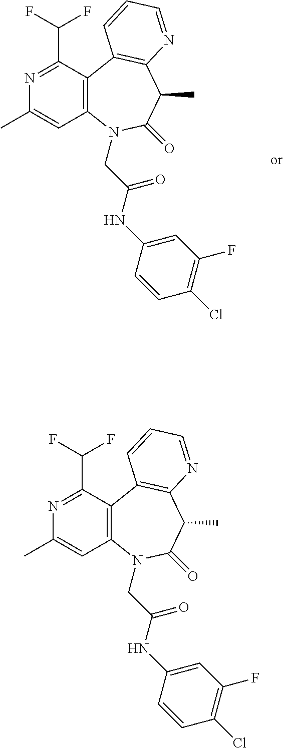 [SMILES: Cc1cc2c(c(C(F)F)n1)-c1cccnc1[C@@H](C)C(=O)N2CC(=O)Nc1ccc(Cl)c(F)c1 ; Cc1cc2c(c(C(F)F)n1)-c1cccnc1[C@H](C)C(=O)N2CC(=O)Nc1ccc(Cl)c(F)c1]

The title compound was prepared according to the general procedure 1 starting from 3-(difluoromethyl)-5,10-dimethyl-4,8,12-triazatricyclo[9.4.0.0^{2,7}]pentadeca-1(11),2(7),3,5,12,14-hexaen-9-one NN202 (273 mg, 0.94 mmol, 100 mass %) and 2-chloro-N-(4-chloro-3-fluoro-phenyl)acetamide (251.4 mg, 1.13 mmol). The residue was purified by preparative basic reverse phase chromatography (YMC Triart-500 g-10 μm-76.5×200 mm using NH₄OH 0.13M in water/MeCN (5 to 85%) as eluent) and then by SFC chiral chromatography (Chiralpak IG from Daicel, CO₂ and 25% EtOH as eluent) to give the title compound (68 mg, yield: 15%) and the second enantiomer (63 mg, Yield: 14%) both as white solids. LC-MS m/z [M+H]⁺: 475; purity: 99%. ¹H NMR (400 MHz, DMSO-d₆) δ 10.56 (s, 1H), 8.69 (d, J=4.8 Hz, 1H), 7.84 (d, J=7.8 Hz, 1H), 7.73 (d, J=11.8 Hz, 1H), 7.53 (dd, J=19.0, 9.8 Hz, 3H), 7.30 (d, J=8.8 Hz, 1H), 6.81 (t, J=53.4 Hz, 1H), 4.66-4.49 (m, 2H), 3.81 (d, J=6.9 Hz, 1H), 2.62 (s, 3H), 1.48 (d, J=6.6 Hz, 3H). Chiral purity: 99%; rt=2.04 min (second eluting enantiomer) column HPLC ChiralPak IG-u with Heptane/EtOH 1:1+0.1% DEA as eluent. For information, first eluting enantiomer rt=1.41 min (99%).

### Example #303: Enantiomer (8S) or (8R) of N-(4-chloro-3-fluoro-phenyl)-2-[5,15-difluoro-8,13-dimethyl-9-oxo-3,10,14-triazatricyclo[9.4.0.0^{2,7}]pentadeca-1(15),2,4,6,11,13-hexaen-10-yl]acetamide

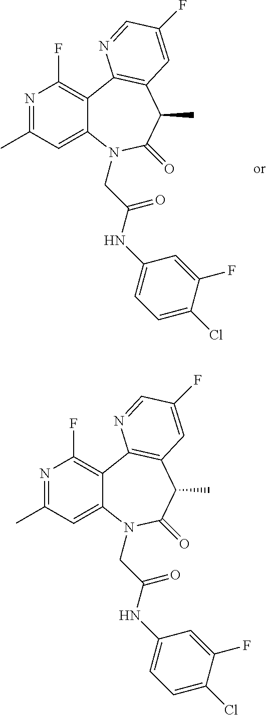 [SMILES: Cc1cc2c(c(F)n1)-c1ncc(F)cc1[C@@H](C)C(=O)N2CC(=O)Nc1ccc(Cl)c(F)c1 ; Cc1cc2c(c(F)n1)-c1ncc(F)cc1[C@H](C)C(=O)N2CC(=O)Nc1ccc(Cl)c(F)c1]

The title product was prepared according to the general procedure 1, starting from 5,15-difluoro-8,13-dimethyl-3,10,14-triazatricyclo[9.4.0.0²⁷]pentadeca-1(11),2(7),3,5,12,14-hexaen-9-one NN332 (78%, 300 mg, 0.85 mmol) and 2-chloro-N-(4-chloro-3-fluoro-phenyl)acetamide (CAS 895641-02-8). After purification by column chromatography on silica gel (using a gradient of 0-100% EtOAc-EtOH 3:1 in isohexane as eluent), the racemate was separated by chiral SFC (column CHIRALPAK IC from Diacel, eluent 25% MeOH, 75% CO₂) to yield the pure enantiomer (283 mg, yield: 21%). LC-MS m/z [M+H]⁺: 461.1, 463.1; purity: 99%. ¹H NMR (400 MHz, DMSO-d₆) δ 10.59 (s, 1H), 8.69 (d, J=2.7 Hz, 1H), 7.81 (dd, J=9.5, 2.8 Hz, 1H), 7.73 (dd, J=11.9, 2.4 Hz, 1H), 7.52 (t, J=8.7 Hz, 1H), 7.34-7.27 (m, 2H), 4.56 (s, 2H), 3.74 (q, J=6.6 Hz, 1H), 2.50 (s, 3H), 1.46 (d, J=6.7 Hz, 3H). ¹⁹F NMR (376 MHz, DMSO-d₆) δ−68.79, −114.66, −126.33. Chiral purity: 99.8%; rt=2.89 min (second eluting enantiomer). For information, first eluting enantiomer rt=2.32 min. Both measured by chiral SFC (column CHIRALPAK IC from Daicel, eluent 25% MeOH+0.1% NH₃, 75% CO₂).

### Example #304: Enantiomer (8S) or (8R) of 2-[5,15-difluoro-8,13-dimethyl-9-oxo-3,10,14-triazatricyclo[9.4.0.0^{2,7}]pentadeca-1(15),2,4,6,11,13-hexaen-10-yl]-N-[4-(difluoromethyl)phenyl]acetamide

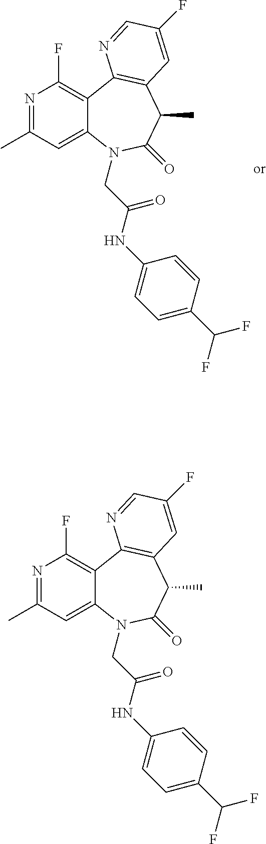 [SMILES: Cc1cc2c(c(F)n1)-c1ncc(F)cc1[C@@H](C)C(=O)N2CC(=O)Nc1ccc(C(F)F)cc1 ; Cc1cc2c(c(F)n1)-c1ncc(F)cc1[C@H](C)C(=O)N2CC(=O)Nc1ccc(C(F)F)cc1]

The title product was prepared according to the general procedure 1, starting from Intermediate NN332 (90 mg, 0.26 mmol) and 2-chloro-N-[4-(difluoromethyl)phenyl]acetamide (CAS 872533-93-2). After purification by column chromatography, the racemate was separated by Chiral SFC (column CHIRALPAK IA from Daicel, eluant 50% MeOH, 50% CO₂) to yield the pure enantiomer as a white solid (17 mg, yield: 34%). LC-MS m/z [M+H]⁺: 459.2; purity: 97%. ¹H NMR (400 MHz, DMSO-d₆) δ 10.47 (s, 1H), 8.70 (d, J=2.7 Hz, 1H), 7.81 (dd, J=9.3, 2.7 Hz, 1H), 7.69 (d, J=8.4 Hz, 2H), 7.52 (d, 2H), 7.33 (s, 1H), 6.97 (t, J=56.1 Hz, 1H), 4.58 (s, 2H), 3.74 (q, J=6.7 Hz, 1H), 1.47 (d, J=6.8 Hz, 3H), 3 protons under solvent signal. Chiral purity: 99%; rt=2.94 min (second eluting enantiomer). For information, first eluting enantiomer rt=0.95 min. Both measured by SFC, CHIRALPAK IA from Daicel, 50% MeOH+0.1% NH₃, 50% CO₂.

### Example #305: N-[4-(difluoromethyl)phenyl]-2-(9-methoxy-5-methyl-6-oxo-5H-pyrido[2,3-d][1]benzazepin-7-yl)acetamide

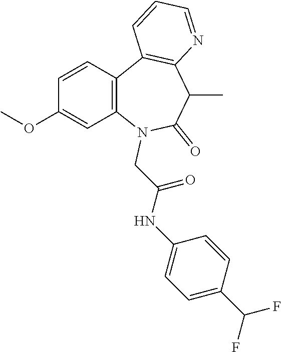 [SMILES: COc1ccc2c(c1)N(CC(=O)Nc1ccc(C(F)F)cc1)C(=O)C(C)c1ncccc1-2]

The title product was prepared according to the general procedure 1, starting from 9-methoxy-5-methyl-5,7-dihydropyrido[2,3-d][1]benzazepin-6-one NN181 (3.9 mg, 0,015 mmol) and 2-chloro-N-[4-(difluoromethyl)phenyl]acetamide (3.7 mg, 0,017 mmol). The reaction mixture was purified by reverse phase HPLC (Purification Method P_B) to afford the title compound as a white solid (0.25 mg, yield: 3%). LC-MS m/z [M+H]⁺: 438.2; purity: 98%.

### Example #306: N-(4-chloro-3-fluoro-phenyl)-2-(9-methoxy-5-methyl-6-oxo-5H-pyrido[2,3-d][1]benzazepin-7-yl)acetamide

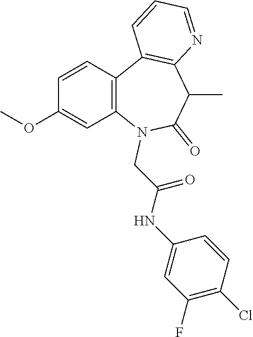 [SMILES: COc1ccc2c(c1)N(CC(=O)Nc1ccc(Cl)c(F)c1)C(=O)C(C)c1ncccc1-2]

The title product was prepared according to the general procedure 1, starting from 9-methoxy-5-methyl-5,7-dihydropyrido[2,3-d][1]benzazepin-6-one NN181 (5 mg, 0.018 mmol) and 2-chloro-N-(4-chloro-3-fluoro-phenyl)acetamide (4.5 mg, 0.020 mmol). The reaction mixture purified by reverse phase HPLC (Purification Method P_B) to afford the title compound (3.9 mg, yield: 48%). LC-MS m/z [M+H]⁺: 440/442; purity: 95%.

### Example #307: Enantiomer (5S) or (5R) of N-(4-chloro-3-fluoro-phenyl)-2-[9-cyano-2-fluoro-5-methyl-6-oxo-5H-pyrido[2,3-d][1]benzazepin-7-yl]acetamid

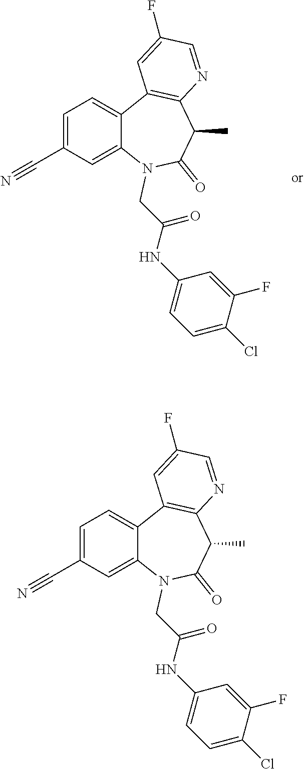 [SMILES: C[C@H]1C(=O)N(CC(=O)Nc2ccc(Cl)c(F)c2)c2cc(C#N)ccc2-c2cc(F)cnc21 ; C[C@@H]1C(=O)N(CC(=O)Nc2ccc(Cl)c(F)c2)c2cc(C#N)ccc2-c2cc(F)cnc21]

The title product was prepared according to the general procedure 1, starting from 2-fluoro-5-methyl-6-oxo-5,7-dihydropyrido[2,3-d][1]benzazepine-9-carbonitrile NN318 (41 mg, 0.15 mmol) and 2-chloro-N-(4-chloro-3-fluoro-phenyl)acetamide (CAS 895641-02-8). After purification by trituration with MeOH, the racemate was separated by chiral SFC (column CHIRALPAK IA from Daicel, eluent 35% MeOH, 65% CO₂) to yield the pure enantiomer (8 mg, yield: 4%) LC-MS m/z [M+H]⁺: 453.1, 455.1; purity: 99%. ¹H NMR (400 MHz, DMSO-d₆) δ 10.55 (s, 1H), 8.72 (d, J=2.8 Hz, 1H), 8.19 (dd, J=9.4, 2.8 Hz, 1H), 8.12-8.04 (m, 1H), 7.98-7.83 (m, 2H), 7.72 (dd, J=11.9, 2.4 Hz, 1H), 7.51 (t, J=8.7 Hz, 1H), 7.30 (dd, J=8.9, 2.4 Hz, 1H), 4.62 (d, J=16.8 Hz, 1H), 4.52 (d, J=16.7 Hz, 1H), 3.65 (q, J=6.5 Hz, 1H), 1.47 (d, J=6.6 Hz, 3H). Chiral purity: 97%; rt=2.51 min (second eluting enantiomer). For information, first eluting enantiomer rt=1.80 min. Both measured by chiral SFC (column CHIRALPAK IA from Daicel, eluent 35% MeOH+0.1% NH₃, 65% CO₂.

### Example #308: Enantiomer (5S) or (5R) of 2-[9-cyano-2-fluoro-5-methyl-6-oxo-5H-pyrido[2,3-d][1]benzazepin-7-yl]-N-[4-(difluoromethyl)phenyl]acetamide

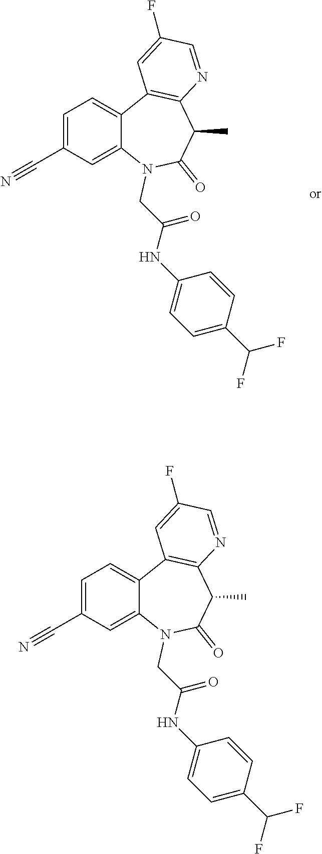 [SMILES: C[C@H]1C(=O)N(CC(=O)Nc2ccc(C(F)F)cc2)c2cc(C#N)ccc2-c2cc(F)cnc21 ; C[C@@H]1C(=O)N(CC(=O)Nc2ccc(C(F)F)cc2)c2cc(C#N)ccc2-c2cc(F)cnc21]

The title product was prepared according to the general procedure 1, starting from Intermediate NN318 (102 mg, 0.38 mmol) and 2-chloro-N-[4-(difluoromethyl)phenyl]acetamide (CAS 872533-93-2). The racemate was separated by Chiral SFC (column CHIRALPAK IG from Daicel, eluant 50% MeOH 50% CO₂) to yield the pure enantiomer as an off white solid (10 mg, yield: 6%). LC-MS m/z [M+H]⁺: 451.1; purity: 99%. ¹H NMR (400 MHz, DMSO-d₆) δ 10.44 (s, 1H), 8.72 (d, J=2.8 Hz, 1H), 8.19 (dd, J=9.4, 2.8 Hz, 1H), 8.11 (d, J=1.5 Hz, 1H), 7.96-7.85 (m, 2H), 7.68 (d, J=8.4 Hz, 2H), 7.51 (d, J=8.4 Hz, 2H), 6.96 (t, J=56.1 Hz, 1H), 4.64 (d, J=16.7 Hz, 1H), 4.55 (d, J=16.7 Hz, 1H), 3.66 (q, J=6.6 Hz, 1H), 1.48 (d, J=6.6 Hz, 3H). Chiral purity: 100%; rt=4.90 min (second eluting enantiomer). For information, first eluting enantiomer rt=2.07 min. Both measured by SFC, CHIRALPAK IG from Daicel, 60% CO₂ and 40% MeOH+0.1% NH₃.

### Example #309: Enantiomer (5S) or (5R) of 2-[9-cyano-11-fluoro-5-methyl-6-oxo-5H-pyrido[2,3-d][1]benzazepin-7-yl]-N-[4-(difluoromethyl)phenyl]acetamide

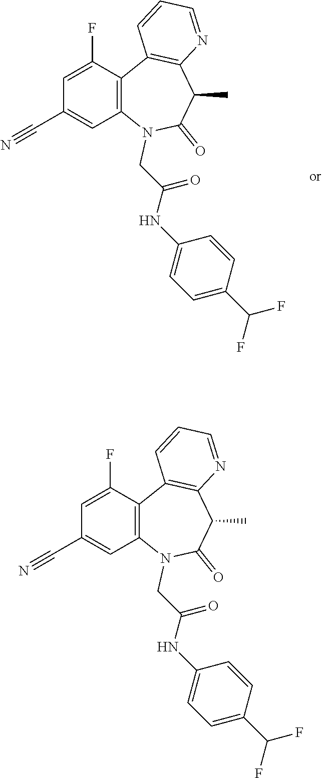 [SMILES: C[C@H]1C(=O)N(CC(=O)Nc2ccc(C(F)F)cc2)c2cc(C#N)cc(F)c2-c2cccnc21 ; C[C@@H]1C(=O)N(CC(=O)Nc2ccc(C(F)F)cc2)c2cc(C#N)cc(F)c2-c2cccnc21]

The title product was prepared according to the general procedure 1, starting from 11-fluoro-5-methyl-6-oxo-5,7-dihydropyrido[2,3-d][1]benzazepine-9-carbonitrile NN326 (58.0 mg, 0.21 mmol) and 2-chloro-N-[4-(difluoromethyl)phenyl]acetamide (CAS 872533-93-2). Following purification by column chromatography on silica gel (using a gradient of 0-8% MeOH in DCM as eluant), the racemate was separated by chiral SFC (column CHIRALPAK IG from Daicel, eluant 35% MeOH+0.03% NH₃, 65% CO₂) to afford the pure enantiomer as an off-white solid (81.0 mg, yield: 83%). LC-MS m/z [M+H]⁺: 451.2; purity: 100%. ¹H NMR (400 MHz, DMSO-d₆) δ 10.44 (s, 1H), 8.71 (dd, J=4.8, 1.7 Hz, 1H), 8.15 (ddd, J=7.9, 4.7, 1.7 Hz, 1H), 7.97 (m, 2H), 7.68 (d, J=8.4 Hz, 2H), 7.56-7.47 (m, 3H), 6.96 (t, J=56.1 Hz, 1H), 4.64 (d, J=16.7 Hz, 1H), 4.53 (d, J=16.8 Hz, 1H), 3.78 (q, J=6.5 Hz, 1H), 1.48 (d, J=6.6 Hz, 3H). Chiral purity: 99.7%; rt=3.47 min (second eluting enantiomer). For information, first eluting enantiomer rt=2.08 min. Both measured by SFC CHIRALPAK IG from Daicel, 40% MeOH+0.03% NH₃, 60% CO₂.

### Example #310: Enantiomer (5S) or (5R) of N-(4-chloro-3-fluoro-phenyl)-2-[9-cyano-11-fluoro-5-methyl-6-oxo-5H-pyrido[2,3-d][1]benzazepin-7-yl]acetamide

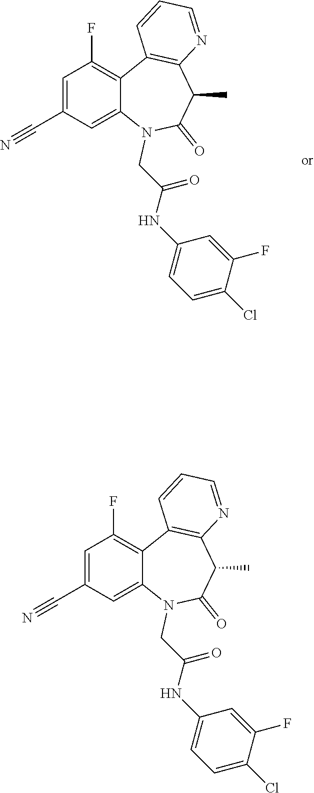 [SMILES: C[C@H]1C(=O)N(CC(=O)Nc2ccc(Cl)c(F)c2)c2cc(C#N)cc(F)c2-c2cccnc21 ; C[C@@H]1C(=O)N(CC(=O)Nc2ccc(Cl)c(F)c2)c2cc(C#N)cc(F)c2-c2cccnc21]

The title product was prepared according to the general procedure 1, starting from Intermediate NN326 (35.0 mg, 0.13 mmol) and 2-chloro-N-(4-chloro-3-fluoro-phenyl)acetamide (CAS 895641-02-8). The crude mixture was purified by reversed phase HPLC ((Purification Method P_B)). The racemate was separated by Chiral SFC (column Lux i-Cellulose 5,from Phenomenex, eluant 30% MeOH 70% CO₂) to yield the pure enantiomer as a white solid (12 mg, yield: 28%). LC-MS m/z [M+H]⁺: 453.1, 455.1; purity: 99%. ¹H NMR (400 MHz, DMSO-d₆) δ 10.56 (s, 1H), 8.71 (dd, J=4.8, 1.6 Hz, 1H), 8.19-8.12 (m, 1H), 8.00-7.93 (m, 2H), 7.72 (dd, J=11.8, 2.3 Hz, 1H), 7.56-7.47 (m, 2H), 7.30 (d, J=9.2 Hz, 1H), 4.62 (d, J=16.8 Hz, 1H), 4.51 (d, J=16.8 Hz, 1H), 3.78 (d, J=6.6 Hz, 1H), 1.48 (d, J=6.6 Hz, 3H). ¹⁹F NMR (376 MHz, DMSO-d₆) δ−112.20, −114.64. Chiral purity: 100%; rt=3.01 min (second eluting enantiomer). For information, first eluting enantiomer rt=2.28 min. Both measured by SFC, CHIRALPAK IC from Daicel, 70% CO₂ and 30% MeOH+0.1% NH₃.

### Example #311: N-(4-chloro-3-fluoro-phenyl)-2-(9-cyano-5-methyl-6-oxo-5H-pyrido[2,3-d][1]benzazepin-7-yl)acetamide

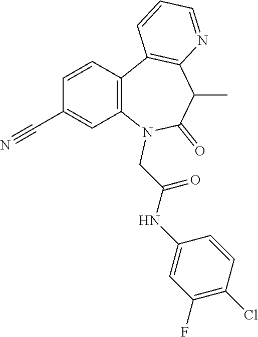 [SMILES: CC1C(=O)N(CC(=O)Nc2ccc(Cl)c(F)c2)c2cc(C#N)ccc2-c2cccnc21]

The title product was prepared according to the general procedure 1, starting from 5-methyl-6-oxo-5,7-dihydropyrido[2,3-d][1]benzazepine-9-carbonitrile NN182 (33 mg, 0,128 mmol) and 2-chloro-N-(4-chloro-3-fluoro-phenyl)acetamide (31 mg, 0,139 mmol) in N,N-dimethylacetamide (0.5 mL). The reaction mixture was purified by reverse phase HPLC (Purification Method P_B) to afford the title compound (10.4 mg, yield: 19%). LC-MS m/z [M+H]⁺: 435/437; purity: 97%. ¹H NMR (400 MHz, DMSO-d₆) δ 10.57 (s, 1H), 8.70 (dd, J=4.8, 1.6 Hz, 1H), 8.16 (dd, J=7.9, 1.7 Hz, 1H), 8.07 (d, J=1.2 Hz, 1H), 7.88 (d, J=2.0 Hz, 2H), 7.74 (dd, J=11.9, 2.4 Hz, 1H), 7.58-7.48 (m, 2H), 7.31 (dd, J=8.8, 2.3 Hz, 1H), 4.56 (q, J=16.7 Hz, 2H), 3.65 (q, J=6.6 Hz, 1H), 1.48 (d, J=6.6 Hz, 3H).

### Example #312: Enantiomer (5S) or (5R) of 2-[9-cyano-5-methyl-6-oxo-5H-pyrido[2,3-d][1]benzazepin-7-yl]-N-[4-(difluoromethyl)phenyl]acetamide

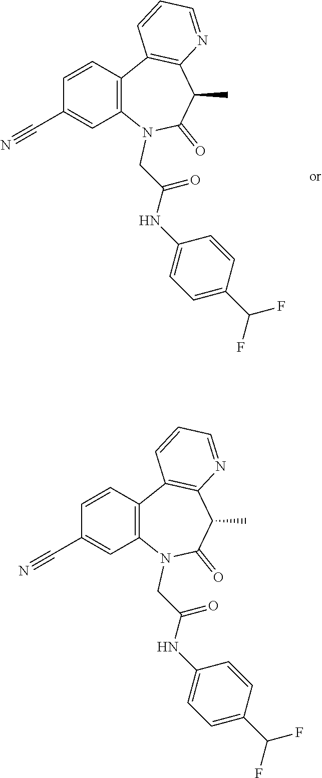 [SMILES: C[C@H]1C(=O)N(CC(=O)Nc2ccc(C(F)F)cc2)c2cc(C#N)ccc2-c2cccnc21 ; C[C@@H]1C(=O)N(CC(=O)Nc2ccc(C(F)F)cc2)c2cc(C#N)ccc2-c2cccnc21]

A mixture of 2-(9-bromo-5-methyl-6-oxo-5H-pyrido[2,3-d][1]benzazepin-7-yl)-N-[4-(difluoromethyl)phenyl]acetamide NN183 (300 mg, 0.6 mmol), tetrakis(triphenylphosphine)palladium(0), zinc cyanide (100 mg, 0.834 mmol) in degassed N,N-dimethylacetamide (3 mL) was stirred and heated at 150° C. under microwave irradiation for 20 min under inert atmosphere. The reaction mixture was neutralized with a saturated NaHCO₃ solution, diluted with AcOEt and filtered through a pad of Celite®. The filtrate was extracted with EtOAc (3×). The combined organic extracts were washed with brine, dried over Na₂SO₄, filtered and concentrated to dryness. After purification by column chromatography on silica gel (using a gradient of 30% to 100% EtOAc in heptane as eluent), the racemate was purified by Chiral HPLC (column CHIRAL NR-R, eluant EtOH-Heptane 1:1) to yield the pure enantiomer. LC-MS m/z [M+H]⁺: 433.0; purity: 100%. Chiral purity: 100%; rt=2.72 min (first eluting enantiomer). For information, second eluting enantiomer rt=3.59 min. Both measured by HPLC, Chiral NR-R, EtOH 50%—Heptane 50%—DEA 0.1%

### Example #313: Enantiomer (5S) or (5R) of 2-[9-chloro-11-fluoro-5-methyl-6-oxo-5H-pyrimido[4,5-d][1]benzazepin-7-yl]-N-[4-(difluoromethyl)phenyl]acetamide

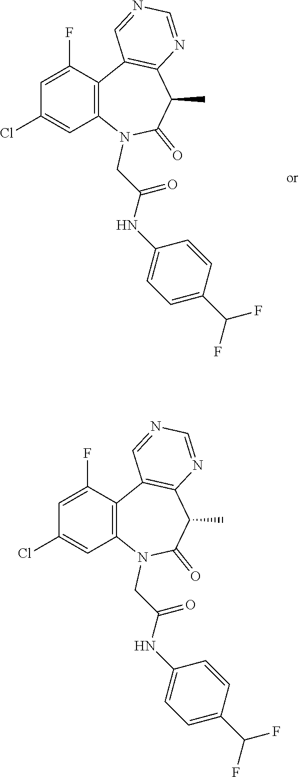 [SMILES: C[C@H]1C(=O)N(CC(=O)Nc2ccc(C(F)F)cc2)c2cc(Cl)cc(F)c2-c2cncnc21 ; C[C@@H]1C(=O)N(CC(=O)Nc2ccc(C(F)F)cc2)c2cc(Cl)cc(F)c2-c2cncnc21]

The title product was prepared according to the general procedure 1, starting from 9-chloro-11-fluoro-5-methyl-5,7-dihydropyrimido[4,5-d][1]benzazepin-6-one NN325 (15 mg, 0.05 mmol) and 2-chloro-N-[4-(difluoromethyl)phenyl]acetamide (CAS 872533-93-2). After complete conversion the reaction mixture was filtered and purified by flash reverse phase chromatography on C18 silica gel (Acidic elution). The racemate was separated by Chiral SFC (column CHIRALPAK IA from Daicel, eluant 35% MeOH+0.1% NH₃, 65% CO₂) to yield the pure enantiomer as a white solid (6 mg, yield: 40%). LC-MS m/z [M+H]⁺: 461.0/463.0; purity: 99%. ¹H NMR (400 MHz, DMSO-d₆) δ 10.44 (s, 1H), 9.26 (s, 1H), 9.10 (d, J=4.6 Hz, 1H), 7.69-7.62 (m, 4H), 7.50 (d, J=8.4 Hz, 2H), 6.96 (t, J=56.1 Hz, 1H), 4.63 (d, J=16.7 Hz, 1H), 4.52 (d, J=16.7 Hz, 1H), 3.89 (q, J=6.4 Hz, 1H), 1.47 (d, J=6.6 Hz, 3H), ¹⁹F NMR (376 MHz, DMSO-d₆) δ−108.13, −113.33. Chiral purity: 100%; rt=2.47 min (second eluting enantiomer). For information, first eluting enantiomer rt=1.57 min. Both measured by SFC, CHIRALPAK IA from Daicel, 35% MeOH+0.1% NH₃, 65% CO₂.

### Example #314: Enantiomer (5S) or (5R) of 2-[9-chloro-11-fluoro-5-methyl-6-oxo-5H-pyrimido[4,5-d][1]benzazepin-7-yl]-N-(4-chloro-3-fluoro-phenyl)acetamide

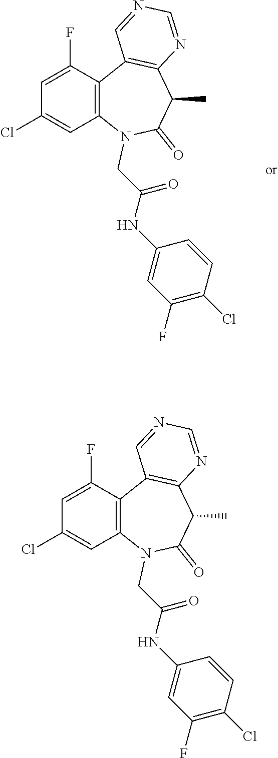 [SMILES: C[C@H]1C(=O)N(CC(=O)Nc2ccc(Cl)c(F)c2)c2cc(Cl)cc(F)c2-c2cncnc21 ; C[C@@H]1C(=O)N(CC(=O)Nc2ccc(Cl)c(F)c2)c2cc(Cl)cc(F)c2-c2cncnc21]

The title product was prepared according to the general procedure 1, starting from 9-chloro-11-fluoro-5-methyl-5,7-dihydropyrimido[4,5-d][1]benzazepin-6-one NN325 (15 mg, 0.05 mmol) and 2-chloro-N-(4-chloro-3-fluoro-phenyl)acetamide (CAS 895641-02-8). After complete conversion the reaction mixture was filtered and purified by flash reverse phase chromatography on C18 silica gel (Acidic elution). The racemate was separated by Chiral SFC (column CHIRALPAK IA from Daicel, eluant 35% MeOH+0.1% NH₃, 65% CO₂) to yield the pure enantiomer as a white solid (7 mg, yield: 38%). LC-MS m/z [M+H]⁺: 463.3, 464.9; rt; purity: 100%. ¹H NMR (400 MHz, DMSO-d₆) δ 10.55 (s, 1H), 9.26 (s, 1H), 9.10 (d, J=4.5 Hz, 1H), 7.73-7.63 (m, 2H), 7.62 (d, J=1.6 Hz, 1H), 7.51 (t, J=8.7 Hz, 1H), 7.29 (dd, J=9.0, 2.3 Hz, 1H), 4.62 (d, J=16.7 Hz, 1H), 4.49 (d, J=16.8 Hz, 1H), 3.89 (q, J=6.5 Hz, 1H), 1.47 (d, J=6.6 Hz, 3H), Chiral purity: 100%; rt=2.70 min (second eluting enantiomer). For information, first eluting enantiomer rt=1.89 min. Both measured by SFC, CHIRALPAK IA from Daicel, 35% MeOH+0.1% NH₃, 65% CO₂.

### Example #315: Enantiomer (5S) or (5R) of N-[4-(difluoromethyl)phenyl]-2-[11-fluoro-9-methoxy-5-methyl-6-oxo-5H-pyrido[2,3-d][1]benzazepin-7-yl]acetamide

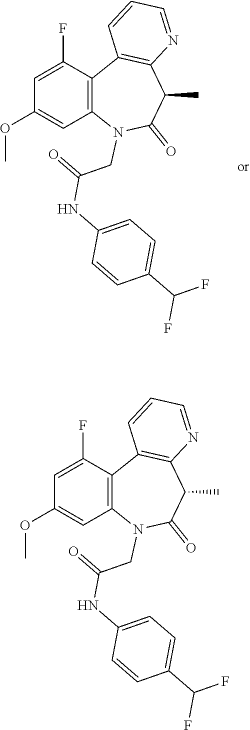 [SMILES: COc1cc(F)c2c(c1)N(CC(=O)Nc1ccc(C(F)F)cc1)C(=O)[C@H](C)c1ncccc1-2 ; COc1cc(F)c2c(c1)N(CC(=O)Nc1ccc(C(F)F)cc1)C(=O)[C@@H](C)c1ncccc1-2]

The title product was prepared according to the general procedure 1, starting from 11-Fluoro-9-methoxy-5-methyl-5,7-dihydropyrido[2,3-d][1]benzazepin-6-one NN333 (40 mg, 0.14 mmol) and 2-chloro-N-[4-(difluoromethyl)phenyl]acetamide (CAS 872533-93-2). The racemate was separated by chiral HPLC with column CHIRALPAK AD 5 μm (10×250 mm) from Daicel, eluent EtOH 100%, at 4.7 mL/min and RT, to yield the pure enantiomer as a white solid (8.5 mg, yield: 28%). Chiral purity: 97%; rt=1.92 min (first eluting enantiomer). For information, second eluting enantiomer rt=3.51 min. Both measured by chiral HPLC with column CHIRALPAK AD-3 (4.6×150 mm) from Daicel, eluent EtOH 100%+0.1% DEA, at 1.5 mL/min and 30° C. LC-MS m/z [M+H]⁺: 456.0; purity: 100%.

### Example #316: Enantiomer (5S) or (5R) of N-(4-chloro-3-fluoro-phenyl)-2-[11-fluoro-9-methoxy-5-methyl-6-oxo-5H-pyrido[2,3-d][1]benzazepin-7-yl]acetamide

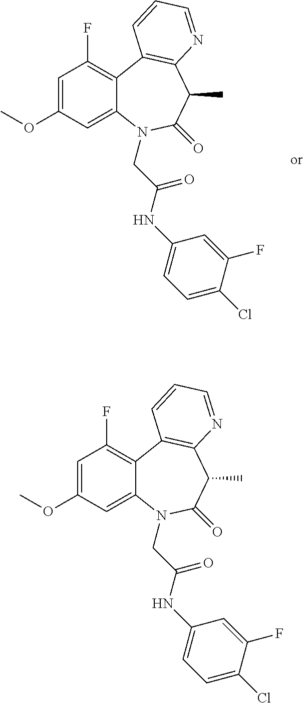 [SMILES: COc1cc(F)c2c(c1)N(CC(=O)Nc1ccc(Cl)c(F)c1)C(=O)[C@H](C)c1ncccc1-2 ; COc1cc(F)c2c(c1)N(CC(=O)Nc1ccc(Cl)c(F)c1)C(=O)[C@@H](C)c1ncccc1-2]

The title product was prepared according to the general procedure 1, starting from 11-Fluoro-9-methoxy-5-methyl-5,7-dihydropyrido[2,3-d][1]benzazepin-6-one NN333 (142 mg, 0.52 mmol) and 2-chloro-N-(4-chloro-3-fluoro-phenyl)acetamide (CAS 895641-02-8). The racemate was separated by Chiral SFC (column CHIRALPAK IG from Daicel, eluant 30% MeOH, 70% CO₂) to yield the pure enantiomer as a white solid (33 mg, yield: 32%). LC-MS m/z [M+H]⁺: 458.1, 460.0; purity: 100%. ¹H NMR (400 MHz, DMSO-d₆) δ 10.52 (s, 1H), 8.61 (dd, J=4.8, 1.7 Hz, 1H), 8.12-7.93 (m, 1H), 7.72 (dd, J=11.9, 2.4 Hz, 1H), 7.51 (t, J=8.7 Hz, 1H), 7.45 (dd, J=7.8, 4.8 Hz, 1H), 7.34-7.25 (m, 1H), 7.10-6.95 (m, 2H), 4.53 (d, J=16.7 Hz, 1H), 4.47 (d, J=16.6 Hz, 1H), 3.85 (s, 3H), 3.71 (q, J=6.6 Hz, 1H), 1.47 (d, J=6.6 Hz, 3H). Chiral purity: 100%; rt=2.96 min (second eluting enantiomer). For information, first eluting enantiomer rt=2.20 min. Both measured by SFC, CHIRALPAK IG from Daicel, 40% MeOH+0.1% NH₃, 60% CO₂.

### Example #317: Enantiomer (5S) or (5R) of N-(4-chloro-3-fluoro-phenyl)-2-[2,9,11-trifluoro-5-hydroxy-6-oxo-5H-pyrido[2,3-d][1]benzazepin-7-yl]acetamide

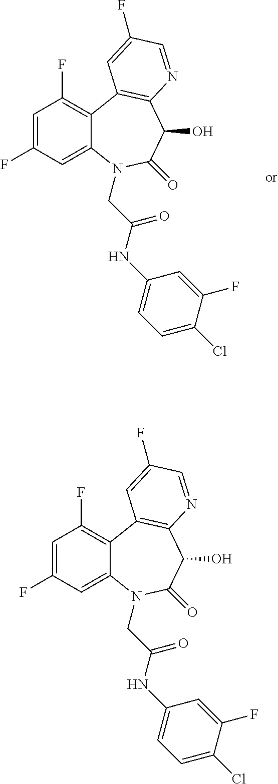 [SMILES: O=C(CN1C(=O)[C@H](O)c2ncc(F)cc2-c2c(F)cc(F)cc21)Nc1ccc(Cl)c(F)c1 ; O=C(CN1C(=O)[C@@H](O)c2ncc(F)cc2-c2c(F)cc(F)cc21)Nc1ccc(Cl)c(F)c1]

The title product was prepared according to the general procedure 1, starting from 2,9,11-Trifluoro-5-hydroxy-5,7-dihydropyrido[2,3-d][1]benzazepin-6-one NN319 (95 mg, 0.369 mmol) and 2-chloro-N-(4-chloro-3-fluoro-phenyl)acetamide (CAS 895641-02-8). After purification by reverse phase HPLC (acidic elution), the racemate was separated by Chiral SFC (column CHIRALPAK IC from Daicel, eluant 50% MeOH, 50% CO₂) to yield the pure enantiomer as an off-white solid (39 mg, yield: 25%). LC-MS m/z [M+H]⁺: 466.1, 468.1; purity: 100%. ¹H NMR (400 MHz, CD30D) δ 8.61 (d, J=2.7 Hz, 1H), 7.99 (ddd, J=9.2, 4.1, 2.7 Hz, 1H), 7.64 (dd, J=11.4, 2.4 Hz, 1H), 7.45-7.30 (m, 2H), 7.28-7.14 (m, 2H), 5.23-5.15 (m, 1H), 4.56 (s, 2H). 2 exchangeable protons not observed. Chiral purity: 100%; rt=3.45 min (second eluting enantiomer). For information, first eluting enantiomer rt=2.07 min. Both measured by HPLC, CHIRALPAK IC from Daicel, 35% MeOH+0.1% NH₃, 65% CO₂.

### Example #318: Enantiomer (5S) or (5R) of N-[4-(difluoromethyl)phenyl]-2-[2,9,11-trifluoro-5-hydroxy-6-oxo-5H-pyrido[2,3-d][1]benzazepin-7-yl]acetamide

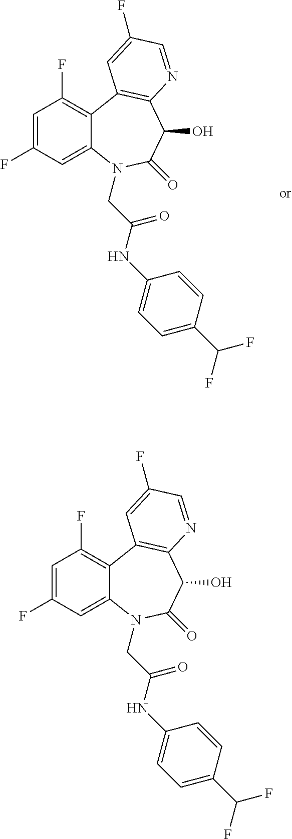 [SMILES: O=C(CN1C(=O)[C@H](O)c2ncc(F)cc2-c2c(F)cc(F)cc21)Nc1ccc(C(F)F)cc1 ; O=C(CN1C(=O)[C@@H](O)c2ncc(F)cc2-c2c(F)cc(F)cc21)Nc1ccc(C(F)F)cc1]

The title product was prepared according to the general procedure 1, starting from 2,9,11-Trifluoro-5-hydroxy-5,7-dihydropyrido[2,3-d][1]benzazepin-6-one NN319 (94 mg, 0.31 mmol) and 2-chloro-N-[4-(difluoromethyl)phenyl]acetamide (CAS 872533-93-2). After purification by column chromatography, the racemate was separated by Chiral SFC (column CHIRALPAK IC from Daicel, eluant 40% MeOH, 60% CO₂) to yield the pure enantiomer as off white solid (1 mg, yield: 1%). LC-MS m/z [M+H]⁺: 464.1; purity: 100%. ¹H NMR (400 MHz, DMSO-d₆) δ 10.45 (s, 1H), 8.73 (d, J=2.7 Hz, 1H), 8.22-8.04 (m, 1H), 7.66 (d, J=8.4 Hz, 2H), 7.56-7.32 (m, 4H), 6.96 (t, J=56.1 Hz, 1H), 5.71 (s, 1H), 5.16 (s, 1H), 4.62 (d, J=16.7 Hz, 1H), 4.53 (d, J=16.7 Hz, 1H). Chiral purity: 91%; rt=2.97 min (second eluting enantiomer). For information, first eluting enantiomer rt=1.70 min. Both measured by SFC, CHIRALPAK IC from Daicel, 35% MeOH+0.1% NH₃, 65% CO₂.

### Example #319: Enantiomer (10S) or (10R) of N-[4-(difluoromethyl)phenyl]-2-[5-methoxy-10-methyl-9-oxo-4,8,12-triazatricyclo[9.4.0.0^{2,7}]pentadeca-1(15),2(7),3,5,11,13-hexaen-8-yl]acetamide

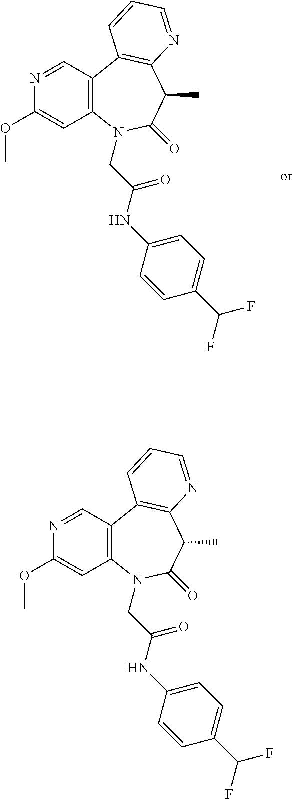 [SMILES: COc1cc2c(cn1)-c1cccnc1[C@@H](C)C(=O)N2CC(=O)Nc1ccc(C(F)F)cc1 ; COc1cc2c(cn1)-c1cccnc1[C@H](C)C(=O)N2CC(=O)Nc1ccc(C(F)F)cc1]

The title product was prepared according to the general procedure 1, starting from 5-methoxy-10-methyl-4,8,12-triazatricyclo[9.4.0.0^{2,7}]pentadeca-1(11),2(7),3,5,12,14-hexaen-9-one NN224 (300 mg, 1.175 mmol) and 2-chloro-N-[4-(difluoromethyl)phenyl]acetamide (271 mg, 1.23 mmol). The racemate was separated by chiral chromatography (SFC Chiralpak IA from Daicel, CO₂+Methanol 30%). Chiral purity: 100%; rt=6.09 min (second eluting enantiomer). For information, first eluting enantiomer rt=2.05 min. Both measured by HPLC, Chiralpak AD from Daicel, Solvent: Ethanol 100%—DEA 0.1%). LC-MS m/z [M+H]⁺: 439.2; purity: 100%.

### Example #320: Enantiomer (10S) or (10R) of N-(4-chloro-3-fluoro-phenyl)-2-[5-methoxy-10-methyl-9-oxo-4,8,12-triazatricyclo[9.4.0.0^{2,7}]pentadeca-1(15),2(7),3,5,11,13-hexaen-8-yl]acetamide

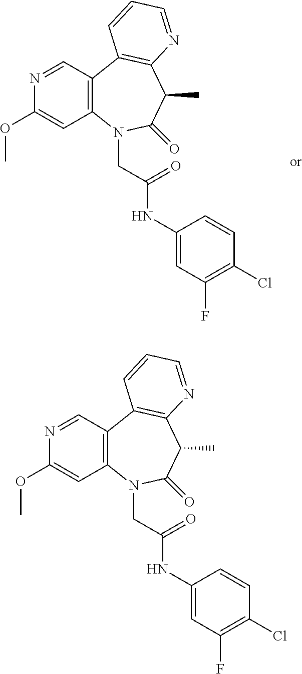 [SMILES: COc1cc2c(cn1)-c1cccnc1[C@@H](C)C(=O)N2CC(=O)Nc1ccc(Cl)c(F)c1 ; COc1cc2c(cn1)-c1cccnc1[C@H](C)C(=O)N2CC(=O)Nc1ccc(Cl)c(F)c1]

The title product was prepared according to the general procedure 1, starting from 5-methoxy-10-methyl-4,8,12-triazatricyclo[9.4.0.0^{2,7}]pentadeca-1(11),2(7),3,5,12,14-hexaen-9-one NN224 and 2-chloro-N-(4-chloro-3-fluoro-phenyl)acetamide (274 mg, 1.234 mmol). The racemate was separated by chiral chromatography (SFC Chiralpak IA from Daicel, CO₂+Ethanol 20%). Chiral purity: 100%; rt=3.18 min (second eluting enantiomer). For information, first eluting enantiomer rt=1.84 min. Both measured by HPLC, Chiralpak IA from Daicel, Solvent: EtOH 100%—DEA 0.1%). LC-MS m/z [M+H]⁺: 441.1/443.1; purity: 100%.

### Example #321: N-(4-chloro-3-fluoro-phenyl)-2-(3-cyclopropyl-1,7-dimethyl-2,6-dioxo-7H-pyrido[3,2-d][3]benzazepin-5-yl)acetamide

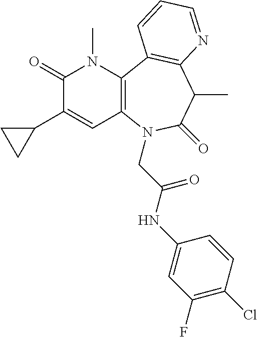 [SMILES: CC1C(=O)N(CC(=O)Nc2ccc(Cl)c(F)c2)c2cc(C3CC3)c(=O)n(C)c2-c2cccnc21]

The title compound was prepared according to the procedure described for example #128 starting from [2-(3-cyclopropyl-1,7-dimethyl-2,6-dioxo-7H-pyrido[3,2-d][3]benzazepin-5-yl)acetyl]oxylithium NN419 (119 mg, 0.33 mmol) and 4-chloro-3-fluoro-aniline (52.1 mg, 0.36 mmol). The crude was purified by column chromatography on silica gel (using a gradient of 0% to 100% EtOAc in isohexane as eluent) to afford the title compound as a light brown powder (31 mg, yield: 19%). LC-MS m/z [M+H]⁺: 480.1, 482.1; purity: 98%. ¹H NMR (400 MHz, DMSO-d₆) δ 10.40 (s, 1H), 7.71-7.68 (m, 1H), 7.67 (d, J=1.8 Hz, 1H), 7.53 (dt, J=17.1, 8.0 Hz, 2H), 7.43 (t, J=7.5 Hz, 1H), 7.38 (d, J=7.9 Hz, 1H), 7.28 (dd, J=8.8, 2.4 Hz, 1H), 7.19 (s, 1H), 4.31 (d, J=16.4 Hz, 1H), 4.18 (d, J=16.4 Hz, 1H), 3.68 (q, J=6.7 Hz, 1H), 3.35 (s, 3H), 2.12 (td, J=8.5, 4.2 Hz, 1H), 1.45 (d, J=6.7 Hz, 3H), 0.93-0.87 (m, 2H), 0.76-0.69 (m, 2H).

### Example #322: 2-(3-cyclopropyl-1,7-dimethyl-2,6-dioxo-7H-pyrido[3,2-d][3]benzazepin-5-yl)-N-[4-(difluoromethyl)phenyl]acetamide

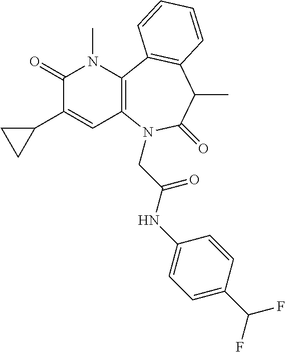 [SMILES: CC1C(=O)N(CC(=O)Nc2ccc(C(F)F)cc2)c2cc(C3CC3)c(=O)n(C)c2-c2ccccc21]

The title compound was prepared according to the procedure described for example #128 starting from [2-(3-cyclopropyl-1,7-dimethyl-2,6-dioxo-7H-pyrido[3,2-d][3]benzazepin-5-yl)acetyl]oxylithium NN419 (77 mg, 0.21 mmol) and 4-(difluoromethyl)aniline hydrochloride (41 mg, 0.23 mmol). The crude product was purified by column chromatography on silica gel (using a gradient of 0-100% EtOAc in isohexane as eluant) to afford the title compound as a light brown solid (45 mg, yield: 44%). LC-MS m/z [M+H]⁺: 478.2; purity: 99%. ¹H NMR (400 MHz, DMSO-d₆) δ 10.27 (s, 1H), 7.67 (dd, J=12.3, 8.3 Hz, 3H), 7.58-7.48 (m, 3H), 7.43 (t, J=7.5 Hz, 1H), 7.38 (d, J=7.9 Hz, 1H), 7.21 (s, 1H), 6.96 (t, J=56 Hz, 1H), 4.34-4.19 (m, 2H), 3.68 (q, J=6.8 Hz, 1H), 3.36 (s, 3H), 2.16-2.07 (m, 1H), 1.45 (d, J=6.8 Hz, 3H), 0.94-0.86 (m, 2H), 0.75-0.68 (m, 2H).

### Example #323: Enantiomer (7S) or (7R) of N-[4-(difluoromethyl)phenyl]-2-[9-fluoro-3,7-dimethyl-6-oxo-7H-pyrido[3,4-a][3]benzazepin-5-yl]acetamide

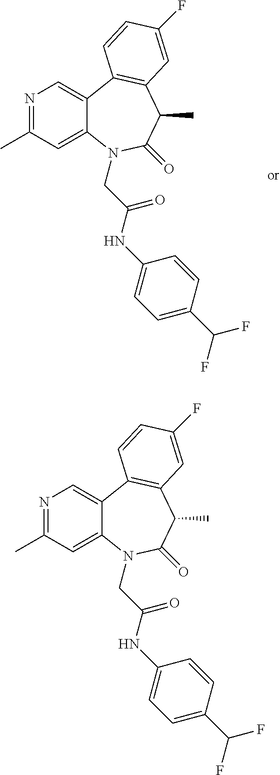

The title product was prepared according to the general procedure 1, starting from 9-Fluoro-3,7-dimethyl-5,7-dihydropyrido[4,3-d][3]benzazepin-6-one NN323 (120 mg, 0.47 mmol) and 2-chloro-N-[4-(difluoromethyl)phenyl]acetamide (CAS 872533-93-2). After purification by column chromatography, the racemate was separated by Chiral SFC (column CHIRALPAK IH from Daicel, eluant 20% MeOH+0.03% NH₃, 80% CO₂, and further purified by reverse phase HPLC (basic elution), to yield the pure enantiomer as white solid (26 mg, yield: 12%). LC-MS m/z [M+H]⁺: 440.1; purity: 100%. ¹H NMR (400 MHz, DMSO-d₆) δ 10.45 (s, 1H), 8.67 (s, 1H), 7.75 (dd, J=8.6, 5.7 Hz, 1H), 7.70 (d, J=8.3 Hz, 2H), 7.52 (d, J=8.2 Hz, 2H), 7.37-7.28 (m, 2H), 7.23 (dd, J=10.1, 2.6 Hz, 1H), 6.97 (t, J=56.1 Hz, 1H), 4.61 (d, J=16.7 Hz, 1H), 4.49 (d, J=16.7 Hz, 1H), 3.47 (q, J=6.7 Hz, 1H), 2.54 (s, 3H), 1.46 (d, J=6.8 Hz, 3H). Chiral purity: 99%; rt=1.90 min (first eluting enantiomer). For information, second eluting enantiomer rt=2.89 min. Both measured by SFC, CHIRALPAK IH from Daicel, 20% MeOH+0.1% NH₃, 80% CO₂.

### Example #324: Enantiomer (7S) or (7R) of N-(4-chloro-3-fluoro-phenyl)-2-[9-fluoro-3,7-dimethyl-6-oxo-7H-pyrido[3,4-a][3]benzazepin-5-yl]acetamide

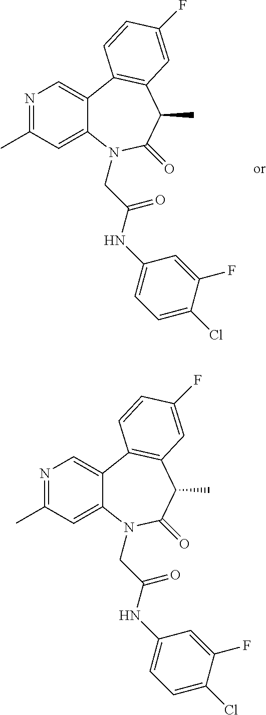 [SMILES: Cc1cc2c(cn1)-c1ccc(F)cc1[C@@H](C)C(=O)N2CC(=O)Nc1ccc(Cl)c(F)c1 ; Cc1cc2c(cn1)-c1ccc(F)cc1[C@H](C)C(=O)N2CC(=O)Nc1ccc(Cl)c(F)c1]

The title product was prepared according to the general procedure 1, starting from 9-Fluoro-3,7-dimethyl-5,7-dihydropyrido[4,3-d][3]benzazepin-6-one NN323 (90 mg, 0.35 mmol) and 2-chloro-N-(4-chloro-3-fluoro-phenyl)acetamide (CAS 895641-02-8). After purification by column chromatography, the racemate was separated by Chiral SFC (column CHIRALPAK IA from Daicel, eluant 30% MeOH, 70% CO₂) to yield the pure enantiomer as colorless solid (42 mg, yield: 35%). LC-MS m/z [M+H]⁺: 442.1, 444.0; purity: 100%. ¹H NMR (400 MHz, DMSO-d₆) δ 10.57 (s, 1H), 8.67 (s, 1H), 7.79-7.71 (m, 2H), 7.52 (t, J=8.7 Hz, 1H), 7.37-7.29 (m, 3H), 7.22 (dd, J=10.3, 2.6 Hz, 1H), 4.58 (d, J=16.8 Hz, 1H), 4.48 (d, J=16.8 Hz, 1H), 3.47 (q, J=6.8 Hz, 1H), 2.54 (s, 3H), 1.45 (d, J=6.8 Hz, 3H). Chiral purity: 99%; rt=2.56 min (second eluting enantiomer). For information, first eluting enantiomer rt=1.56 min. Both measured by SFC, CHIRALPAK IA from Daicel, 35% MeOH+0.1% NH₃, 65% CO₂.

### Example #325: Enantiomer (10S) or (10R) of N-[4-(difluoromethyl)phenyl]-2-[14-fluoro-5-(1-fluorocyclopropyl)-10-methyl-9-oxo-4,8,12-triazatricyclo[9.4.0.0^{2,7}]pentadeca-1(11),2,4,6,12,14-hexaen-8-yl]acetamide

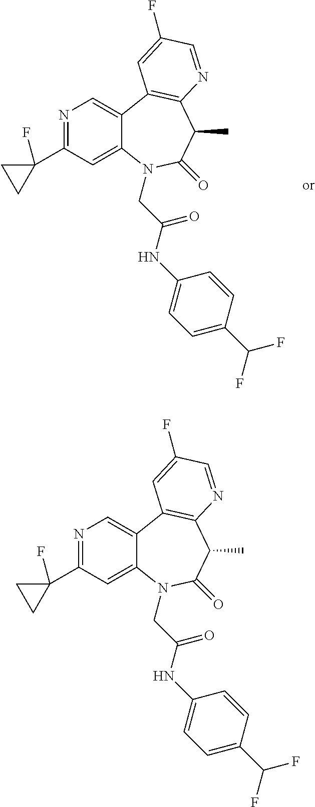 [SMILES: C[C@H]1C(=O)N(CC(=O)Nc2ccc(C(F)F)cc2)c2cc(C3(F)CC3)ncc2-c2cc(F)cnc21 ; C[C@@H]1C(=O)N(CC(=O)Nc2ccc(C(F)F)cc2)c2cc(C3(F)CC3)ncc2-c2cc(F)cnc21]

The title product was prepared according to the general procedure 1, starting from 14-fluoro-5-(1-fluorocyclopropyl)-10-methyl-4,8,12-triazatricyclo[9.4.0.0^{2,7}]pentadeca-1(11),2(7),3,5,12,14-hexaen-9-one NN324 (80%, 60 mg, 0.159 mmol) and 2-chloro-N-[4-(difluoromethyl)phenyl]acetamide (CAS 872533-93-2) (39 mg, 0.17 mmol). After purification by reverse phase HPLC (Purification Method P_B), the racemate was separated by Chiral SFC (column CHIRALPAK IG from Daicel, eluant 50% MeOH+0.1% NH₃, 50% CO₂) to yield the pure enantiomer as a white solid (15.8 mg, yield: 45%). LC-MS m/z [M+H]⁺: 485.1; purity: 99%. ¹H NMR (400 MHz, DMSO-d₆) δ 10.52 (s, 1H), 8.82 (d, J=1.1 Hz, 1H), 8.71 (d, J=2.7 Hz, 1H), 8.22 (dd, J=9.5, 2.8 Hz, 1H), 7.69 (dd, J=4.9, 3.6 Hz, 3H), 7.52 (d, J=8.4 Hz, 2H), 6.97 (t, J=56.1 Hz, 1H), 4.69 (d, J=16.8 Hz, 1H), 4.63 (d, J=16.9 Hz, 1H), 3.71 (q, J=6.5 Hz, 1H), 1.65-1.56 (m, 2H), 1.50 (d, J=6.6 Hz, 3H), 1.44 (dd, J=12.4, 8.9 Hz, 2H)¹⁹F NMR (376 MHz, DMSO-d₆) δ−108.14, −129.63, −191.82. Chiral purity: 99.83%; rt=4.13 min (second eluting enantiomer). For information, first eluting enantiomer rt=1.23 min. Both measured by chiral SFC, CHIRALPAK AY-H from Daicel, 45% MeOH+0.1% NH₃, 55% CO₂.

### Example #326: Enantiomer (10S) or (10R) of N-(4-chloro-3-fluoro-phenyl)-2-[14-fluoro-5-(1-fluorocyclopropyl)-10-methyl-9-oxo-4,8,12-triazatricyclo[9.4.0.0^{2,7}]pentadeca-1(15),2,4,6,11,13-hexaen-8-yl]acetamide

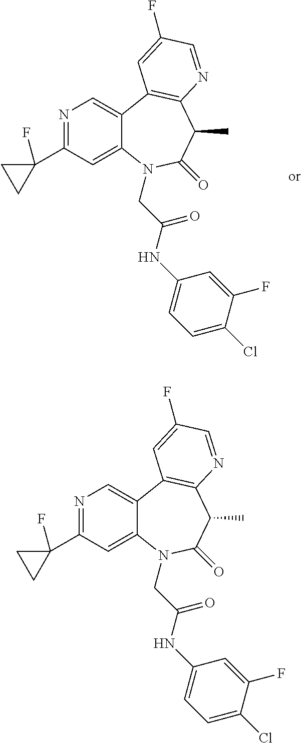 [SMILES: C[C@H]1C(=O)N(CC(=O)Nc2ccc(Cl)c(F)c2)c2cc(C3(F)CC3)ncc2-c2cc(F)cnc21 ; C[C@@H]1C(=O)N(CC(=O)Nc2ccc(Cl)c(F)c2)c2cc(C3(F)CC3)ncc2-c2cc(F)cnc21]

The title product was prepared according to the general procedure 1, starting from 14-fluoro-5-(1-fluorocyclopropyl)-10-methyl-4,8,12-triazatricyclo[9.4.0.0^{2,7}]pentadeca-1(11),2(7),3,5,12,14-hexaen-9-one NN324 (60 mg, 0.16 mmol) and 2-chloro-N-(4-chloro-3-fluoro-phenyl)acetamide (CAS 895641-02-8). The residue was purified by reverse phase chromatography (Purification Method P_B). The racemate was separated by Chiral SFC (column CHIRALPAK IG from Daicel, eluant 50% MeOH 50% CO₂) to yield the pure enantiomer as a white solid (15 mg, yield: 44%). LC-MS m/z [M+H]⁺: 487.1, 489.0; purity: 99%. ¹H NMR (400 MHz, DMSO-d₆) δ 10.63 (s, 1H), 8.82 (d, J=1.2 Hz, 1H), 8.71 (d, J=2.8 Hz, 1H), 8.22 (dd, J=9.5, 2.8 Hz, 1H), 7.72 (dd, J=11.8, 2.4 Hz, 1H), 7.68 (d, J=1.4 Hz, 1H), 7.52 (t, J=8.7 Hz, 1H), 7.36-7.27 (m, 1H), 4.65 (s, 2H), 3.71 (q, J=6.6 Hz, 1H), 1.60 (d, J=19.0 Hz, 2H), 1.49 (d, J=6.6 Hz, 3H), 1.44 (dd, J=12.1, 9.0 Hz, 2H). ¹⁹F NMR (376 MHz, DMSO-d₆) δ−114.57, −129.61, −191.80. Chiral purity: 100%; rt=3.69 min (second eluting enantiomer). For information, first eluting enantiomer rt=1.32 min. Both measured by SFC, CHIRALPAK IG from Daicel, 55% CO₂ and 45% MeOH+0.1% NH₃.

### Example #327: Enantiomer (10S) or (10R) of N-(4-chloro-3-fluoro-phenyl)-2-[14-fluoro-5,10-dimethyl-9-oxo-4,8,12-triazatricyclo[9.4.0.0^{2,7}]pentadeca-1(15),2,4,6,11,13-hexaen-8-yl]acetamide

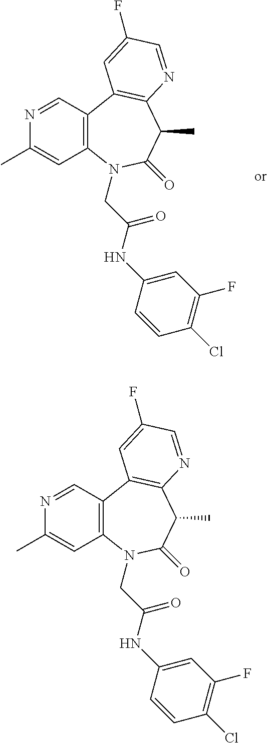 [SMILES: Cc1cc2c(cn1)-c1cc(F)cnc1[C@@H](C)C(=O)N2CC(=O)Nc1ccc(Cl)c(F)c1 ; Cc1cc2c(cn1)-c1cc(F)cnc1[C@H](C)C(=O)N2CC(=O)Nc1ccc(Cl)c(F)c1]

The title product was prepared according to the general procedure 1, starting from 14-Fluoro-5,10-dimethyl-4,8,12-triazatricyclo[9.4.0.0^{2,7}]pentadeca-1(11),2,4,6,12,14-hexaen-9-one NN320 (59 mg, 0.23 mmol) and 2-chloro-N-(4-chloro-3-fluoro-phenyl)acetamide (72 mg, 0.32 mmol). The residue was purified by basic reverse phase chromatography (YMC Triart-500 g-10 μm-76.5×200 mm using NH₄OH 0.13M in water/MeCN (5 to 85%) as eluent) and preparative HPLC (Waters XBridge OBD MS C18 column using NH₄HCO₃ 10 mM+50 μl/L NH OH in water/MeCN (0 to 100%) as eluent) and then by liquid chiral chromatography (Chiralpak AD from Daicel, using Heptane-EtOH 1:1 as solvents) to give the compound as a white solid (13 mg, Yield: 13%) and the other enantiomer as white solid (13 mg, Yield=13%). LC-MS m/z [M+H]⁺: 443; purity: 99%. ¹H NMR (400 MHz, DMSO-d₆) δ 10.59 (s, 1H), 8.76 (s, 1H), 8.69 (d, J=2.7 Hz, 1H), 8.20 (dd, J=9.4, 2.8 Hz, 1H), 7.74 (dd, J=11.8, 2.4 Hz, 1H), 7.52 (t, J=8.6 Hz, 1H), 7.39 (s, 1H), 7.34-7.29 (m, 1H), 4.64-4.50 (m, 2H), 3.65 (q, J=6.6 Hz, 1H), 2.56 (s, 3H), 1.48 (d, J=6.6 Hz, 3H).Chiral purity: 100%; rt=2.79 min (second eluting enantiomer) column UHPLC ChiralPak AD with Heptane/EtOH 1:1+0.1% DEA as eluent. For information, first eluting enantiomer rt=1.65 min (100%).

### Example #328: Enantiomer (10S) or (10R) of N-[4-(difluoromethyl)phenyl]-2-[14-fluoro-5,10-dimethyl-9-oxo-4,8,12-triazatricyclo[9.4.0.0^{2,7}]pentadeca-1(15),2,4,6,11,13-hexaen-8-yl]acetamide

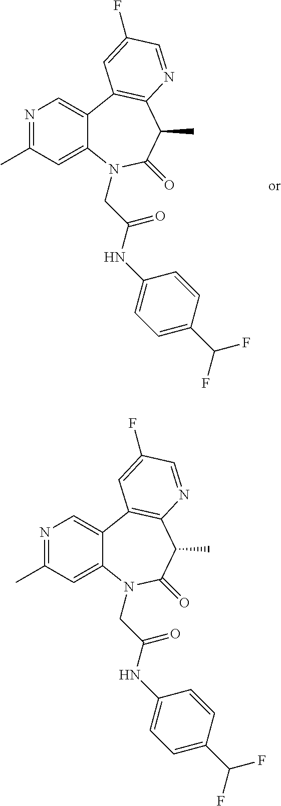 [SMILES: Cc1cc2c(cn1)-c1cc(F)cnc1[C@@H](C)C(=O)N2CC(=O)Nc1ccc(C(F)F)cc1 ; Cc1cc2c(cn1)-c1cc(F)cnc1[C@H](C)C(=O)N2CC(=O)Nc1ccc(C(F)F)cc1]

The title product was prepared according to the general procedure 1, starting from 14-Fluoro-5,10-dimethyl-4,8,12-triazatricyclo[9.4.0.0^{2,7}]pentadeca-1(11),2,4,6,12,14-hexaen-9-one NN320 (95 mg, 0.369 mmol) and 2-chloro-N-[4-(difluoromethyl)phenyl]acetamide (CAS 872533-93-2). After purification by reverse phase HPLC (Purification Method P_A), the racemate was separated by Chiral SFC (column CHIRALPAK IA from Daicel, eluant 40% MeOH, 60% CO₂) to yield the pure enantiomer as a colorless solid (50 mg, yield: 37%). LC-MS m/z [M+H]⁺: 441.2; purity: 100%. ¹H NMR (400 MHz, DMSO-d₆) δ 10.46 (s, 1H), 8.76 (s, 1H), 8.68 (d, J=2.8 Hz, 1H), 8.20 (dd, J=9.6, 2.8 Hz, 1H), 7.69 (d, J=8.4 Hz, 2H), 7.51 (d, J=8.4 Hz, 2H), 7.40 (s, 1H), 6.96 (t, J=56.1 Hz, 1H), 4.63 (d, J=16.8 Hz, 1H), 4.56 (d, J=16.7 Hz, 1H), 3.65 (q, J=6.5 Hz, 1H), 2.56 (s, 3H), 1.49 (d, J=6.6 Hz, 3H). Chiral purity: 99%; rt=3.01 min (second eluting enantiomer). For information, first eluting enantiomer rt=1.40 min. Both measured by HPLC, CHIRALPAK IA from Daicel, 30% MeOH+0.1% NH₃, 70% CO₂.

### Example #329: Atropisomer aS or aR of enantiomer (10S) or (10R) of 2-[5-(difluoromethoxy)-3-methoxy-10-methyl-9-oxo-4,8,12-triazatricyclo[9.4.0.0^{2,7}]pentadeca-1(15),2(7),3,5,11,13-hexaen-8-yl]-N-[4-(difluoromethyl)phenyl]acetamide

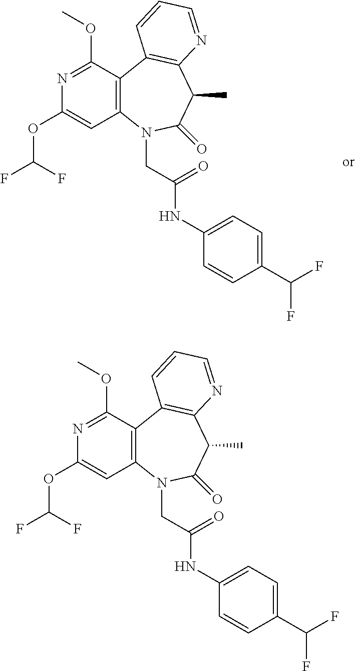 [SMILES: COc1nc(OC(F)F)cc2c1-c1cccnc1[C@@H](C)C(=O)N2CC(=O)Nc1ccc(C(F)F)cc1 ; COc1nc(OC(F)F)cc2c1-c1cccnc1[C@H](C)C(=O)N2CC(=O)Nc1ccc(C(F)F)cc1]

The title compound was prepared according to the general procedure 1, starting from 5-(difluoromethoxy)-3-methoxy-10-methyl-4,8,12-triazatricyclo[9.4.0.0^{2,7}]pentadeca-1(11),2(7),3,5,12,14-hexaen-9-one NN162 (67.2 mg, 0.21 mmol) and 2-chloro-N-[4-(difluoromethyl)phenyl]acetamide (55.0 mg, 0.25 mmol). The reaction mixture of atropisomers obtained (one minor and one major) was separated by SFC (Daicel DCpak P4VP, CO₂+EtOH 5% to 50%). The racemate of the minor atropisomer was separated by Chiral HPLC (Reprosil Chiral NR-R from Dr Maisch, iPrOH 50%—heptane 50%) to afford the title compound (2.9 mg, yield: 3%) as a white solid. LC-MS m/z: [M+H]⁺: 505.1; purity: 100%. ¹H NMR (400 MHz, DMSO-d₆) δ 10.46 (s, 1H), 8.61 (dd, J=4.8, 1.7 Hz, 1H), 8.07 (dd, J=7.9, 1.7 Hz, 1H), 7.82 (t, J=72.3 Hz, 1H), 7.67 (d, J=8.4 Hz, 2H), 7.54-7.46 (m, 3H), 6.96 (t, J=56.0 Hz, 1H), 6.82 (s, 1H), 4.63-4.52 (m, 2H), 3.94 (s, 3H), 3.80 (q, J=6.6 Hz, 1H), 1.48 (d, J=6.6 Hz, 3H). ¹⁹F NMR (376 MHz, DMSO-d₆) δ−86.23-−88.47 (m), −108.09 (d, J=56.0 Hz). Chiral purity: 100%; rt=2.97 min (first eluting enantiomer). For information, second eluting enantiomer rt=4.43 min. Both measured by HPLC (Reprosil Chiral NR-R from Dr Maisch, iPrOH 50%—heptane 50%—DEA 0.1%).

### Example #330: Atropisomer aR or aS of enantiomer (10S) or (10R) of 2-[5-(difluoromethoxy)-3-methoxy-10-methyl-9-oxo-4,8,12-triazatricyclo[9.4.0.0^{2,7}]pentadeca-1(15),2(7),3,5,11,13-hexaen-8-yl]-N-[4-(difluoromethyl)phenyl]acetamide

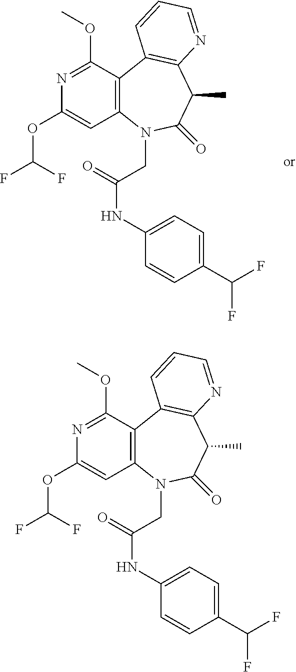 [SMILES: COc1nc(OC(F)F)cc2c1-c1cccnc1[C@@H](C)C(=O)N2CC(=O)Nc1ccc(C(F)F)cc1 ; COc1nc(OC(F)F)cc2c1-c1cccnc1[C@H](C)C(=O)N2CC(=O)Nc1ccc(C(F)F)cc1]

The racemate of the major atropisomer from the previous example was separated by Chiral HPLC (Reprosil Chiral NR-R from Dr Maisch, iPrOH 50%—heptane 50%) to afford the title compound (15.2 mg, yield: 14%) as a white solid. LC-MS m/z: [M+H]⁺: 505.1; purity: 100%. ¹H NMR (400 MHz, DMSO-d₆) δ 10.46 (s, 1H), 8.60 (dd, J=4.8, 1.7 Hz, 1H), 8.15 (dd, J=8.0, 1.7 Hz, 1H), 7.96 (t, J=72.7 Hz, 1H), 7.68 (d, J=8.3 Hz, 2H), 7.51 (d, J=8.3 Hz, 2H), 7.44 (dd, J=8.0, 4.8 Hz, 1H), 6.97 (t, J=56.2 Hz, 1H), 6.82 (s, 1H), 4.64-4.48 (m, 2H), 3.94 (s, 3H), 3.73 (q, J=6.6 Hz, 1H), 1.47 (d, J=6.6 Hz, 3H). ¹⁹F NMR (376 MHz, DMSO-d₆) δ−86.23-−87.85 (m), −108.08 (d, J=56.0 Hz). Chiral purity: 99%; rt=3.17 min (first eluting enantiomer). For information, second eluting enantiomer rt=4.60 min. Both measured by HPLC (Reprosil Chiral NR-R from Dr Maisch, iPrOH 50%—heptane 50%—DEA 0.1%).

### Example #331: Enantiomer (5S) or (5R) of 2-[9-chloro-11-fluoro-5-methyl-6-oxo-5H-pyrido[2,3-d][1]benzazepin-7-yl]-N-[6-(trifluoromethyl)-3-pyridyl]acetamide

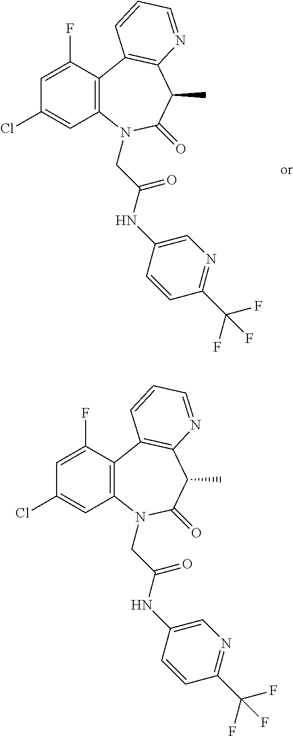 [SMILES: C[C@H]1C(=O)N(CC(=O)Nc2ccc(C(F)(F)F)nc2)c2cc(Cl)cc(F)c2-c2cccnc21 ; C[C@@H]1C(=O)N(CC(=O)Nc2ccc(C(F)(F)F)nc2)c2cc(Cl)cc(F)c2-c2cccnc21]

The title compound was prepared according to the general procedure 1, starting from 9-chloro-11-fluoro-5-methyl-5,7-dihydropyrido[2,3-d][1]benzazepin-6-one NN217 (138 mg, 0.5 mmol) and 2-chloro-N-[6-(trifluoromethyl)-3-pyridyl]acetamide NN193 (125 mg, 0.52 mmol). The racemate was separated by chiral chromatography (SFC Chiralpak IC from Daicel, CO₂+Methanol 20%). Chiral purity: 100%; rt=1.54 min (first eluting enantiomer). For information, second eluting enantiomer rt=1.86 min. Both measured by HPLC, Chiralpak IC from Daicel, Solvent: ACN 100%—DEA 0.1%). LC-MS m/z [M+H]⁺: 479.2/481.2; purity: 97.8% to give the title compound (91.7 mg) as a solid. ¹H NMR (400 MHz, DMSO-d₆) δ 10.86 (s, 1H), 8.83 (s, 1H), 8.67 (d, J=4.8 Hz, 1H), 8.30 (d, J=9.1 Hz, 1H), 8.13-8.06 (m, 1H), 7.86 (d, J=8.6 Hz, 1H), 7.63-7.56 (massif, 2H), 7.50 (dd, J=8.0, 4.9 Hz, 1H), 4.70-4.43 (m, 2H), 3.82-3.73 (m, 1H), 1.48 (d, J=6.6 Hz, 3H).

### Example #332: Enantiomer (5S) or (5R) of 2-[9-chloro-11-fluoro-5-methyl-6-oxo-5H-pyrido[2,3-d][1]benzazepin-7-yl]-N-[4-(difluoromethyl)phenyl]acetamide

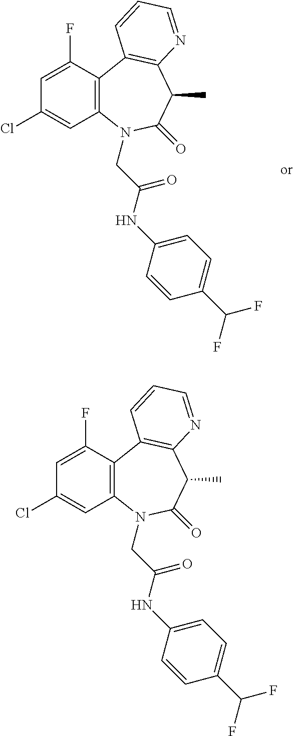 [SMILES: C[C@H]1C(=O)N(CC(=O)Nc2ccc(C(F)F)cc2)c2cc(Cl)cc(F)c2-c2cccnc21 ; C[C@@H]1C(=O)N(CC(=O)Nc2ccc(C(F)F)cc2)c2cc(Cl)cc(F)c2-c2cccnc21]

The title compound was prepared according to the general procedure 1, starting from 9-chloro-11-fluoro-5-methyl-5,7-dihydropyrido[2,3-d][1]benzazepin-6-one NN217 (138 mg, 0.5 mmol) and 2-chloro-N-[4-(difluoromethyl)phenyl]acetamide (115 mg, 0.52 mmol). The racemate was separated by chiral chromatography (SFC Reprosil Chiral NR-R from Dr Maisch, CO₂+Methanol 20%). Chiral purity: 100%; rt=2.058 min (first eluting enantiomer). For information, second eluting enantiomer rt=2.721 min. Both measured by HPLC, Reprosil Chiral NR-R from Dr Maisch, Solvent: Heptane 50%—EtOH 50%—DEA 0.1%). LC-MS m/z [M+H]⁺: 460.2/462.2; purity: 99.4% to give the title compound (90 mg) as a solid. ¹H NMR (400 MHz, DMSO-d₆) δ 10.46 (s, 1H), 8.67 (dd, J=4.8, 1.7 Hz, 1H), 8.14-8.06 (m, 1H), 7.70 (s, 1H), 7.68 (s, 1H), 7.62-7.56 (m, 2H), 7.55-7.47 (m, 3H), 6.97 (t, J=56.1 Hz, 1H), 4.55 (dd, 2H), 3.76 (q, J=6.5 Hz, 1H), 1.48 (d, J=6.6 Hz, 3H).

### Example #333: Enantiomer (5S) or (5R) of 2-[9-(3,3-difluoroazetidin-1-yl)-5-methyl-6-oxo-5H-pyrido[2,3-d][1]benzazepin-7-yl]-N-[6-(trifluoromethyl)-3-pyridyl]acetamide

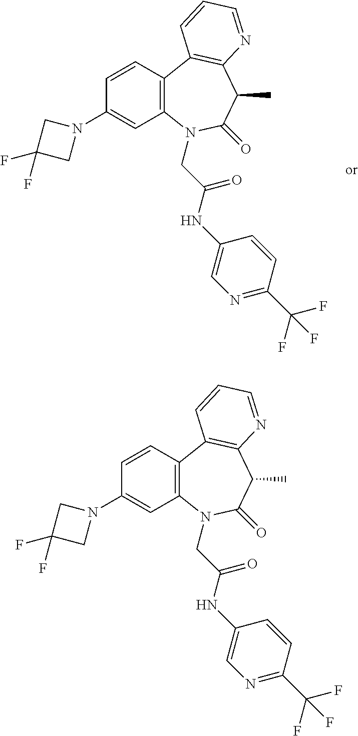 [SMILES: C[C@H]1C(=O)N(CC(=O)Nc2ccc(C(F)(F)F)nc2)c2cc(N3CC(F)(F)C3)ccc2-c2cccnc21 ; C[C@@H]1C(=O)N(CC(=O)Nc2ccc(C(F)(F)F)nc2)c2cc(N3CC(F)(F)C3)ccc2-c2cccnc21]

The title compound was prepared according to the general procedure 1, starting from 9-(3,3-difluoroazetidin-1-yl)-5-methyl-5,7-dihydropyrido[2,3-d][1]benzazepin-6-one NN184 (100 mg, 0.32 mmol) and 2-chloro-N-[6-(trifluoromethyl)-3-pyridyl]acetamide NN193 (90.8 mg, 0.38 mmol). The crude mixture was purified by reverse phase HPLC (Purification Method P_B) to afford the racemate (45.1 mg, yield: 25%). ¹H NMR (500 MHz, DMSO-d₆) δ 10.78 (s, 1H), 8.82 (d, J=2.4 Hz, 1H), 8.57 (dd, J=4.7, 1.7 Hz, 1H), 8.28 (dd, J=8.6, 2.5 Hz, 1H), 7.99 (dd, J=7.8, 1.7 Hz, 1H), 7.86 (d, J=8.6 Hz, 1H), 7.53 (d, J=8.5 Hz, 1H), 7.45 (dd, J=7.8, 4.8 Hz, 1H), 6.70 (d, J=2.4 Hz, 1H), 6.66 (dd, J=8.5, 2.3 Hz, 1H), 4.63-4.51 (m, 2H), 4.37 (dtd, J=41.4, 12.2, 10.0 Hz, 4H), 3.60 (q, J=6.7 Hz, 1H), 1.47 (d, J=6.7 Hz, 3H). The racemate was separated by Chiral SFC (column CHIRALPAK IC, CO₂+Methanol 30%) to yield the pure enantiomer (13.5 mg, yield: 8.2%). LC-MS m/z [M+H]⁺: 518; purity: 98%. Chiral purity: 100%; rt=2.03 min (first eluting enantiomer). For information, second eluting enantiomer rt=2.43 min. Both measured by HPLC, Chiralpak IA, iso-Propanol 50%—Heptane 50%—DEA 0.1%

### Example #334: Enantiomer (5S) or (5R) of 2-[9-(3,3-difluoroazetidin-1-yl)-5-methyl-6-oxo-5H-pyrido[2,3-d][1]benzazepin-7-yl]-N-[4-(difluoromethyl)phenyl]acetamide

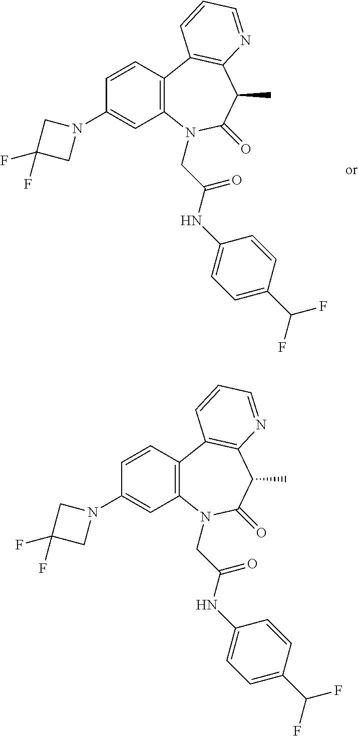 [SMILES: C[C@H]1C(=O)N(CC(=O)Nc2ccc(C(F)F)cc2)c2cc(N3CC(F)(F)C3)ccc2-c2cccnc21 ; C[C@@H]1C(=O)N(CC(=O)Nc2ccc(C(F)F)cc2)c2cc(N3CC(F)(F)C3)ccc2-c2cccnc21]

The title compound was prepared according to the general procedure 1, starting from 9-(3,3-difluoroazetidin-1-yl)-5-methyl-5,7-dihydropyrido[2,3-d][1]benzazepin-6-one NN184 (100 mg, 0.32 mmol) and 2-chloro-N-[4-(difluoromethyl)phenyl]acetamide (84 mg, 0.38 mmol). The crude was purified by reverse phase HPLC (Method P_B) to afford the racemate title compound (65 mg, yield: 41%). ¹H NMR (500 MHz, DMSO-d₆) δ 10.35 (s, 1H), 8.57 (dd, J=4.8, 1.7 Hz, 1H), 7.98 (dd, J=7.8, 1.7 Hz, 1H), 7.67 (d, J=8.4 Hz, 2H), 7.51 (t, J=8.7 Hz, 3H), 7.45 (dd, J=7.8, 4.8 Hz, 1H), 6.96 (t, J=56.1 Hz, 1H), 6.72 (d, J=2.4 Hz, 1H), 6.65 (dd, J=8.5, 2.4 Hz, 1H), 4.57-4.48 (m, 2H), 4.37 (dtd, J=40.0, 12.2, 10.0 Hz, 4H), 3.59 (d, J=6.8 Hz, 1H), 1.47 (d, J=6.7 Hz, 3H). The racemate was separated by Chiral HPLC (column CHIRAL NR-R, eluant: 100% EtOH) to yield the pure enantiomer (19.3 mg, yield: 12%). LC-MS m/z [M+H]⁺: 499.1; purity: 100%. Chiral purity: 100%; rt=1.88 min (first eluting enantiomer). For information, second eluting enantiomer rt=2.22 min. Both measured by HPLC, Chiral NR-R, EtOH 100%—DEA 0.1%

### Example #335: Enantiomer (10S) or (10R) of N-[4-(difluoromethyl)phenyl]-2-[3,5-dimethoxy-10-methyl-9-oxo-4,8,12-triazatricyclo[9.4.0.0^{2,7}]pentadeca-1(15),2(7),3,5,11,13-hexaen-8-yl]acetamide

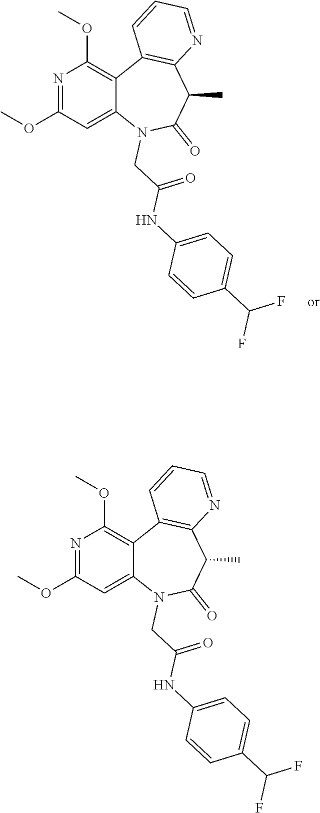 [SMILES: COc1cc2c(c(OC)n1)-c1cccnc1[C@@H](C)C(=O)N2CC(=O)Nc1ccc(C(F)F)cc1 ; COc1cc2c(c(OC)n1)-c1cccnc1[C@H](C)C(=O)N2CC(=O)Nc1ccc(C(F)F)cc1]

The title product was prepared according to the General Procedure 1, starting from 3,5-dimethoxy-10-methyl-4,8,12-triazatricyclo[9.4.0.0^{2,7}]pentadeca-1 (11),2(7),3,5,12,14-hexaen-9-one NN192 (1.7 g, 6 mmol) and 2-chloro-N-[4-(difluoromethyl)phenyl]acetamide (1.44 g, 6.56 mmol). The yellow solid (racemate, 1.6 g) was separated by chiral SFC with column Chiralpak OD-I 20 μm (50×290 mm) from Daicel, eluent CO₂₊₂₀% EtOH, at 360 mL/min, 30° C. and BPR 150 bar to yield the pure enantiomer as a white solid (580 mg, yield: 21%). Chiral purity: 98%; rt=2.46 min (second eluting enantiomer). For information, first eluting enantiomer rt=1.82 min. Both measured by chiral HPLC with column Chiralpak IB-3(4.6×150 mm) from Daicel, eluent iPrOH:n-heptane 1:1+0.1% DEA, at 1.5 mL/min and 30° C. LC-MS m/z [M+H]⁺: 469.10; purity: 90%. ¹H NMR (400 MHz, DMSO-d₆) δ 10.43 (s, 1H), 8.55 (dd, J=4.8, 1.8 Hz, 1H), 8.11 (dd, J=7.9, 1.7 Hz, 1H), 7.68 (d, J=8.4 Hz, 2H), 7.51 (d, J=8.4 Hz, 2H), 7.41 (dd, J=7.9, 4.7 Hz, 1H), 6.97 (7, J=55.4 Hz, 1H), 6.54 (s, 1H), 4.58-4.45 (m, 2H), 3.94 (s, 6H), 3.69 (q, J=6.6 Hz, 1H), 1.46 (d, J=6.7 Hz, 3H).

### Example #336: Enantiomer (10S) or (10R) of 2-[3,5-dimethoxy-10-methyl-9-oxo-4,8,12-triazatricyclo[9.4.0.0^{2,7}]pentadeca-1(15),2(7),3,5,11,13-hexaen-8-yl]-N-[5-(trifluoromethyl)-2-pyridyl]acetamide

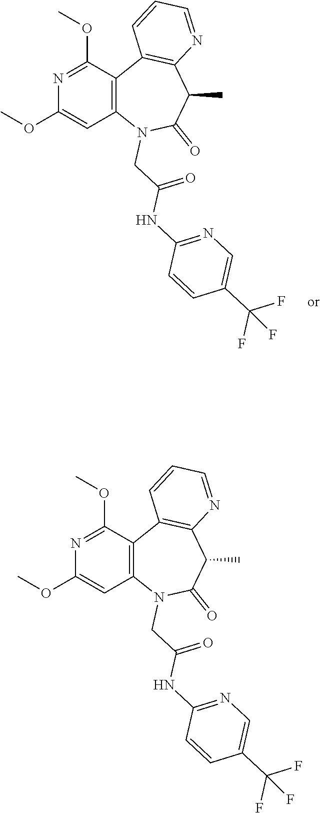 [SMILES: COc1cc2c(c(OC)n1)-c1cccnc1[C@@H](C)C(=O)N2CC(=O)Nc1ccc(C(F)(F)F)cn1 ; COc1cc2c(c(OC)n1)-c1cccnc1[C@H](C)C(=O)N2CC(=O)Nc1ccc(C(F)(F)F)cn1]

The title product was prepared according to the General Procedure 1, starting from 3,5-dimethoxy-10-methyl-4,8,12-triazatricyclo[9.4.0.0^{2,7}]pentadeca-1(11),2(7),3,5,12,14-hexaen-9-one NN192 (143 mg, 0.501 mmol) and 2-chloro-N-[5-(trifluoromethyl)-2-pyridyl]acetamide NN194 (125 mg, 0.524 mmol). The racemate was separated by chiral chromatography (SFC Chiralpak IG from Daicel, CO₂+Methanol 40%). Chiral purity: 99.9%; rt=2.28 min (second eluting enantiomer). For information, first eluting enantiomer rt=1.69 min. Both measured by HPLC, Chiralpak IG from Daicel, Solvent: Methanol 100%—DEA 0.1%). LC-MS m/z [M+H]⁺: 488.1; purity: 98% to give the title compound (20.5 mg) as a solid.

### Example #337: Enantiomer (10S) or (10R) of 2-[3,5-dimethoxy-10-methyl-9-oxo-4,8,12-triazatricyclo[9.4.0.0^{2,7}]pentadeca-1(15),2(7),3,5,11,13-hexaen-8-yl]-N-[6-(trifluoromethyl)-3-pyridyl]acetamide

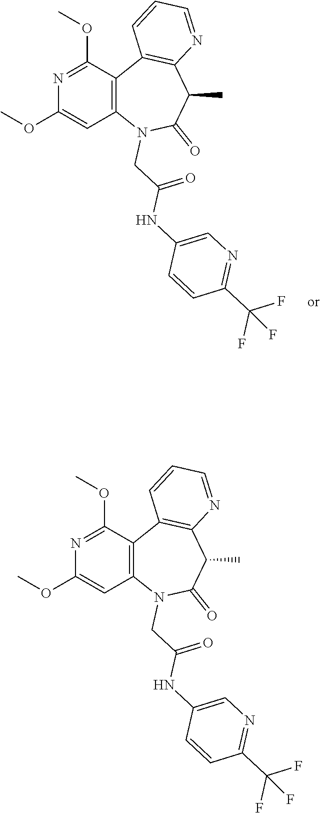 [SMILES: COc1cc2c(c(OC)n1)-c1cccnc1[C@@H](C)C(=O)N2CC(=O)Nc1ccc(C(F)(F)F)nc1 ; COc1cc2c(c(OC)n1)-c1cccnc1[C@H](C)C(=O)N2CC(=O)Nc1ccc(C(F)(F)F)nc1]

The title product was prepared according to the General Procedure 1, starting from 3,5-dimethoxy-10-methyl-4,8,12-triazatricyclo[9.4.0.0^{2,7}]pentadeca-1(11),2(7),3,5,12,14-hexaen-9-one NN192 (143 mg, 0.5 mmol) and 2-chloro-N-[6-(trifluoromethyl)-3-pyridyl]acetamide NN193 (125 mg, 0.52 mmol). The racemate was separated by chiral chromatography (SFC Chiralpak IA, CO₂+Ethanol 20%). Chiral purity: 100%; rt=2.60 min (second eluting enantiomer). For information, first eluting enantiomer rt=1.86 min. Both measured by HPLC, Chiralpak IA from Daicel, Solvent: EtOH 50%—Heptane 50%—DEA 0.1%). LC-MS m/z [M+H]⁺: 488.02; purity: 100% to give the title compound (15.8 mg) as a solid.

### Example #338: 2-[3-chloro-5-(difluoromethyl)-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide

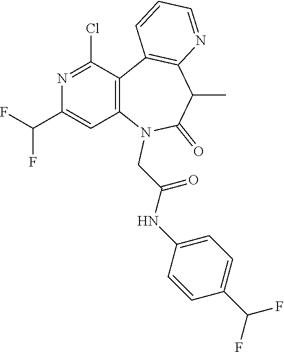 [SMILES: CC1C(=O)N(CC(=O)Nc2ccc(C(F)F)cc2)c2cc(C(F)F)nc(Cl)c2-c2cccnc21]

The title product was prepared according to general procedure 1 starting from 3-chloro-5-(difluoromethyl)-10-methyl-4,8,12-triazatricyclo[9.4.0.0^{2,7}]pentadeca-1(11),2(7),3,5,12,14-hexaen-9-one NN207 (1.25 mg, 0.004 mmol) and 2-chloro-N-[4-(difluoromethyl)phenyl]acetamide (1.06 mg, 0.007 mmol). The reaction mixture was concentrated under vacuo and purified by reverse phase chromatography (Purification Method P_B) to give the title compound (0.64 mg, yield: 82%) as a white solid. LC-MS m/z: [M+H]⁺: 493/495; purity: 95.5%.

### Example #339: 2-[1-chloro-3-(difluoromethyl)-6-oxo-7H-pyrido[4,3-d][3]benzazepin-5-yl]-N-[4-(difluoromethyl)phenyl]acetamide

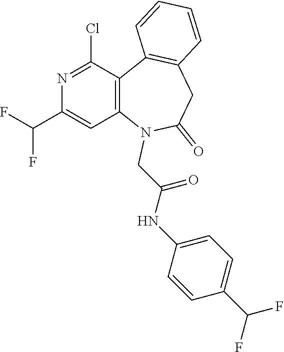 [SMILES: O=C(CN1C(=O)Cc2ccccc2-c2c1cc(C(F)F)nc2Cl)Nc1ccc(C(F)F)cc1]

The title product was prepared according to general procedure 1 from 1-chloro-3-(difluoromethyl)-5,7-dihydropyrido[4,3-d][3]benzazepin-6-one NN205 (0.48 mg 0.0016 mmol) and 2-chloro-N-[4-(difluoromethyl)phenyl]acetamide (0.5 mg, 0.002 mmol). The reaction mixture was concentrated under vacuo and purified by reverse phase chromatography basic to give the title compound (0.64 mg, yield: 82%) as a white solid. LC-MS m/z: [M+H]⁺: 478; purity: 99.5%.

### Example #340: 2-(1,9-dichloro-3-cyclopropyl-6-oxo-7H-pyrido[4,3-d][3]benzazepin-5-yl)-N-[4-(difluoromethyl)phenyl]acetamide

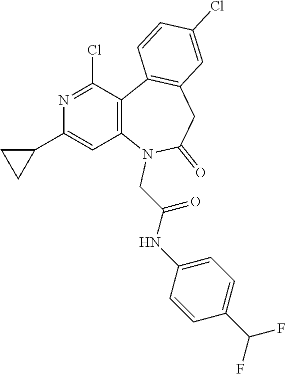 [SMILES: O=C(CN1C(=O)Cc2cc(Cl)ccc2-c2c1cc(C1CC1)nc2Cl)Nc1ccc(C(F)F)cc1]

The title product was prepared according to general procedure 1 starting from 1,9-dichloro-3-cyclopropyl-5,7-dihydropyrido[4,3-d][3]benzazepin-6-one NN203 (0.58 mg 0.0018 mmol) and 2-chloro-N-[4-(difluoromethyl)phenyl]acetamide (0.5 mg, 0.002 mmol). The reaction mixture was concentrated under vacuo and purified by reverse phase chromatography (Method P_B) to give the title compound (0.17 mg, yield: 19%) as a white solid. LC-MS m/z: [M+H]⁺: 502/504; purity: 100%.

### Example #341: N-(4-chloro-3-fluoro-phenyl)-2-[9-(difluoromethyl)-5-methyl-6-oxo-5H-pyrido[2,3-d][1]benzazepin-7-yl]acetamide

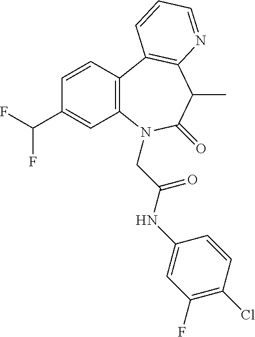 [SMILES: CC1C(=O)N(CC(=O)Nc2ccc(Cl)c(F)c2)c2cc(C(F)F)ccc2-c2cccnc21]

The title product was prepared according to general procedure 1 starting from 9-(difluoromethyl)-5-methyl-5,7-dihydropyrido[2,3-d][1]benzazepin-6-one NN186 (11.4 mg, 0.029 mmol) and 2-chloro-N-(4-chloro-3-fluoro-phenyl)acetamide (7 mg, 0.031 mmol. The reaction mixture was purified by reverse phase HPLC (Purification Method P_B) to afford the title compound (10 mg, yield: 76%). LC-MS m/z [M+H]⁺: 460/462; purity: 91%. ¹H NMR (400 MHz, DMSO-d₆) δ 10.56 (s, 1H), 8.68 (dd, J=4.8, 1.6 Hz, 1H), 8.13 (dd, J=7.8, 1.7 Hz, 1H), 7.83 (d, J=8.1 Hz, 1H), 7.79 (s, 1H), 7.74 (dd, J=11.9, 2.4 Hz, 1H), 7.61 (d, J=8.1 Hz, 1H), 7.57-7.48 (m, 2H), 7.32 (dd, J=8.8, 2.4 Hz, 1H), 7.15 (t, J=55.6 Hz, 1H), 4.60-4.42 (m, 2H), 3.64 (q, J=6.6 Hz, 1H), 1.49 (d, J=6.6 Hz, 3H).

### Example #342: Enantiomer (10S) or (10R) of N-(4-chloro-3-fluoro-phenyl)-2-[5-(difluoromethyl)-10-methyl-9-oxo-4,8,12-triazatricyclo[9.4.0.0^{2,7}]pentadeca-1(15),2,4,6,11,13-hexaen-8-yl]acetamide

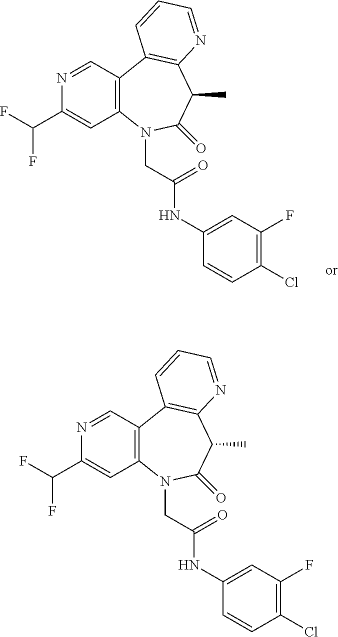 [SMILES: C[C@H]1C(=O)N(CC(=O)Nc2ccc(Cl)c(F)c2)c2cc(C(F)F)ncc2-c2cccnc21 ; C[C@@H]1C(=O)N(CC(=O)Nc2ccc(Cl)c(F)c2)c2cc(C(F)F)ncc2-c2cccnc21]

The title product was prepared according to general procedure 1 starting from 5-(difluoromethyl)-10-methyl-4,8,12-triazatricyclo[9.4.0.0^{2,7}]pentadeca-1(11),2(7),3,5,12,14-hexaen-9-one NN222 (275 mg, 0,9993 mmol) and 2-chloro-N-(4-chloro-3-fluoro-phenyl)acetamide (233 mg, 1.05 mmol). The racemate was separated by chiral chromatography (SFC Chiralpak AD from Daicel, Heptane 50%—Ethanol 50%—DEA 0.1%). Chiral purity: 100%; rt=3.45 min (second eluting enantiomer). For information, first eluting enantiomer rt=1.96 min. Both measured by HPLC, Chiralpak AD from Daicel, Solvent: Heptane 50%—Ethanol 50%—DEA 0.1%). LC-MS m/z [M+H]⁺: 460.9/462.9; purity: 100% to give the title compound (72 mg) as a solid. ¹H NMR (400 MHz, DMSO-d₆) δ 10.65 (s, 1H), 8.99 (s, 1H), 8.73 (dd, J=4.9, 1.6 Hz, 1H), 8.26 (dd, J=7.9, 1.6 Hz, 1H), 7.80 (s, 1H), 7.73 (dd, J=11.8, 2.4 Hz, 1H), 7.57 (dd, J=7.8, 4.8 Hz, 1H), 7.53 (dd, J=8.7, 8.7 Hz, 1H), 7.32 (dd, J=8.8, 2.3 Hz, 1H), 7.08 (t, J=54.7 Hz, 1H), 4.64 (s, 2H), 3.71 (q, J=6.6 Hz, 1H), 1.50 (d, J=6.6 Hz, 3H).

### Example #343: N-(4-chloro-3-fluoro-phenyl)-2-[9-[(3-hydroxy-3-methyl-azetidin-1-yl)methyl]-5-methyl-6-oxo-5H-pyrido[2,3d][1]benzazepin-7-yl]acetamide

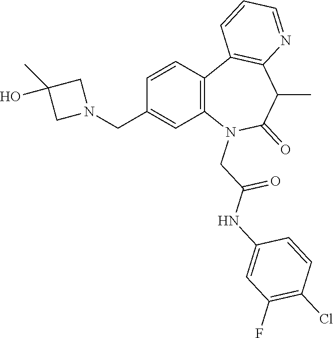 [SMILES: CC1C(=O)N(CC(=O)Nc2ccc(Cl)c(F)c2)c2cc(CN3CC(C)(O)C3)ccc2-c2cccnc21]

To a suspension of N-(4-chloro-3-fluoro-phenyl)-2-(9-formyl-5-methyl-6-oxo-5H-pyrido[2,3-d][1]benzazepin-7-yl)acetamide NN188 (15 mg, 0,034 mmol) and 3-methylazetidin-3-ol hydrochloride (5.4 mg, 0.042 mmol) in THF (0.25 mL) and EtOH (0.25 mL) was added acetic acid (25 μL, 0.436 mmol) followed by MP-Cyanoborohydride resin (50.6 mg, 0,103 mmol, 2.03 mmol/g) at room temperature. The reaction mixture was agitated and heated at 60° C. for 18 h. After cooling to room temperature, the reaction mixture was filtered, and the polymer rinsed with THF (3×). The filtrate was concentrated to dryness. The crude was purified by reverse phase HPLC (Purification Method P_B) to afford the title compound as a white solid (4.1 mg, yield: 24%). LC-MS m/z [M+H]⁺: 509/511; purity: 95%.

### Example #344: N-(4-chloro-3-fluoro-phenyl)-2-[9-[(3,3-difluoroazetidin-1-yl)methyl]-5-methyl-6-oxo-5H-pyrido[2,3-d][1]benzazepin-7-yl]acetamide

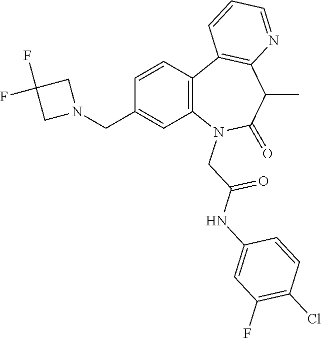 [SMILES: CC1C(=O)N(CC(=O)Nc2ccc(Cl)c(F)c2)c2cc(CN3CC(F)(F)C3)ccc2-c2cccnc21]

The title compound was prepared according to the same procedure as for example #343 with 3,3-difluoroazetidine. The crude mixture was purified by reverse phase HPLC (Purification Method P_B) to afford the title compound (4.8 mg, yield: 27%). LC-MS m/z [M+H]⁺: 515/517; purity: 93%.

### Example #345: N-(4-chloro-3-fluoro-phenyl)-2-[9-[1-(2,2-difluoroethyl)azetidin-3-yl]-11-fluoro-5-methyl-6-oxo-5H-pyrido[2,3-d][1]benzazepin-7-yl]acetamide

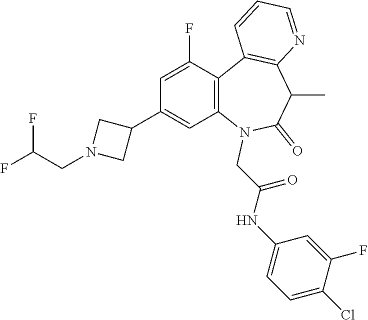 [SMILES: CC1C(=O)N(CC(=O)Nc2ccc(Cl)c(F)c2)c2cc(C3CN(CC(F)F)C3)cc(F)c2-c2cccnc21]

Step 1: Synthesis of tert-butyl 3-[7-(2-ethoxy-2-oxo-ethyl)-11-fluoro-5-methyl-6-oxo-5H-pyrido[2,3-d][1]benzazepin-9-yl]azetidine-1-carboxylate #345_1

A solution of ethyl 2-(9-chloro-11-fluoro-5-methyl-6-oxo-5H-pyrido[2,3-d][1]benzazepin-7-yl)acetate NN383 (0.50 g, 1.31 mmol), copper iodide (10 mg, 0.052 mmol) and [1,1′-bis(diphenylphosphino)ferrocene]dichloropalladium(II) (77 mg, 0.11 mmol) in anhydrous DMA (5 mL) was degassed with nitrogen. The reaction mixture was then heated to 85° C. under an atmosphere of nitrogen. (1-tert-butoxycarbonylazetidin-3-yl)-iodo-zinc (0.29 M in THF, 20.6 mL, 5.89 mmol) was added and the reaction mixture stirred for 1 h at 85° C. The reaction was quenched with sat. aq. NH₄Cl (10 mL) and diluted with EtOAc (50 mL). The organic phase was separated and washed with LiCl 1M (2×20 mL) then with brine (2×20 mL). The organic phase was dried over Na₂SO₄, filtered and concentrated under reduced pressure. The crude was purified by column chromatography on silica gel (using a gradient of 0-100% EtOAc in isohexane as eluent) to afford the title compound (335 mg, yield: 50%). LC-MS m/z [M+H]⁺: 483.9; purity: 94%.

### Step 2: Synthesis of ethyl 2-[9-(azetidin-3-yl)-11-fluoro-5-methyl-6-oxo-5H-pyrido[2,3-d][1]benzazepin-7-yl]acetate #345_2

Tert-butyl 3-[7-(2-ethoxy-2-oxo-ethyl)-11-fluoro-5-methyl-6-oxo-5H-pyrido[2,3-d][1]benzazepin-9-yl]azetidine-1-carboxylate #345_1 (305 mg, 0.631 mmol) was dissolved in DCM (5 mL) and 4 M HCl in 1,4-dioxane (4 M, 3.15 mL, 12.6 mmol) was added. The reaction mixture was stirred for 1 h then sat. aq. NaHCO₃ was added until pH 8 was reached. The aqueous phase was then extracted with DCM (2×25 mL). The combined organics were dried over Na₂SO₄ then concentrated under vacuo to afford the title compound as a beige gum (164 mg, yield: 37%). LC-MS (Method 3) m/z [M+H]⁺: 384.4; rt: 0.47 min; purity: 54%.

### Step 3: Synthesis of ethyl 2-[9-[1-(2,2-difluoroethyl)azetidin-3-yl]-11-fluoro-5-methyl-6-oxo-5H-pyrido[2,3-d][1]benzazepin-7-yl]acetate #345_3

Ethyl 2-[9-(azetidin-3-yl)-11-fluoro-5-methyl-6-oxo-5H-pyrido[2,3-d][1]benzazepin-7-yl]acetate #345_2 (54% purity, 164 mg, 0.23 mmol) was dissolved in DMF (3 mL) and K2CO₃ (120 mg, 0.868 mmol) and 1,1-difluoro-2-iodo-ethane (41.2 μL, 0.468 mmol) were added. The reaction mixture was stirred at 50° C. for 18 h. Additional K2CO₃ (60 mg, 0.434 mmol) and 1,1-difluoro-2-iodo-ethane (41.2 μL, 0.468 mmol) were added and the reaction mixture was stirred at 50° C. for 1 h. Additional 1,1-difluoro-2-iodo-ethane (41.2 μL, 0.468 mmol) was added and the reaction mixture was stirred for a further 1 h. Water (25 mL) was added and the aqueous phase was extracted with EtOAc (3×25 mL). The combined organics were washed with brine (2×50 mL), dried over Na₂SO₄, filtered and concentrated under vacuum. The crude was purified by column chromatography on silica gel (using a gradient of 0-100% EtOAc in isohexane as eluent) to afford the title compound (43.9 mg, yield: 40%). LC-MS m/z [M+H]⁺: 447.8; ¹H NMR (400 MHz, CD30D) δ 8.62 (dd, J=4.8, 1.7 Hz, 1H), 8.12 (ddd, J=7.9, 4.4, 1.7 Hz, 1H), 7.47 (dd, J=7.9, 4.8 Hz, 1H), 7.40-7.27 (m, 2H), 5.90 (tt, J=55.8, 4.2 Hz, 1H), 4.59 (d, J=17.3 Hz, 1H), 4.48 (d, J=17.3 Hz, 1H), 4.17-4.01 (m, 2H), 3.96-3.83 (m, 3H), 3.77 (q, J=6.7 Hz, 1H), 3.48 (q, J=5.0 Hz, 2H), 2.97 (td, J=15.7, 4.2 Hz, 2H), 1.63 (d, J=6.7 Hz, 3H), 1.13 (t, J=7.1 Hz, 3H); purity: 95%.

### Step 4: Synthesis of N-(4-chloro-3-fluoro-phenyl)-2-[9-[1-(2,2-difluoroethyl)azetidin-3-yl]-11-fluoro-5-methyl-6-oxo-5H-pyrido[2,3-d][1]benzazepin-7-yl]acetamide #345

To a solution of ethyl 2-[9-[1-(2,2-difluoroethyl)azetidin-3-yl]-11-fluoro-5-methyl-6-oxo-5H-pyrido[2,3-d][1]benzazepin-7-yl]acetate #345_3 (44 mg, 0.09 mmol) in THF-MeOH-water 2:2:1, (1.25 mL) was added LiOH·H₂O (8.2 mg, 0.196 mmol) and the reaction mixture was stirred for 2 h at RT. The reaction mixture was concentrated under vacuo and co-evaporated with MeCN affording a light beige solid. The solid was re-dissolved in DMF (2 mL) and 4-chloro-3-fluoro-aniline (17 mg, 0.12 mmol), N,N-diisopropylethylamine (0.051 mL, 0.29 mmol) and HATU (56 mg, 0.15 mmol) were sequentially added. The reaction mixture was stirred at RT for 18 h. Water (25 mL) was added and the aqueous phase was extracted with EtOAc (2×25 mL). The combined organics were washed with brine (3×25 mL), then dried over Na₂SO₄, filtered and concentrated under vacuo. The crude was purified by flash chromatography on silica gel (using a gradient of 0-100% EtOAc in isohexane as eluent) and then by preparative HPLC (Purification Method P_B) to afford the title compound as off-white solid (2.0 mg, yield: 4%). LC-MS (Method 3) m/z [M+H]⁺: 547.1, 549.2; rt: 2.03 min; purity: 100%. ¹H NMR (400 MHz, CD30D) δ 8.63 (dd, J=4.9, 1.7 Hz, 1H), 8.14 (ddd, J=7.9, 4.4, 1.7 Hz, 1H), 7.71 (dd, J=11.5, 2.4 Hz, 1H), 7.52-7.23 (m, 5H), 5.86 (tt, J=55.8, 4.2 Hz, 1H), 4.60 (d, J=16.5 Hz, 1H), 4.46 (d, J=16.5 Hz, 1H), 3.95-3.73 (m, 4H), 3.50-3.41 (m, 2H), 2.94 (td, J=15.6, 4.2 Hz, 2H), 1.65 (d, J=6.7 Hz, 3H). One exchangeable proton not observed. ¹⁹F NMR (376 MHz, CD30D) δ−116.07-−116.28 (m), −116.71-−116.96 (m), −122.90 (dt, J=55.8, 15.6 Hz).

### Example #346: N-[4-(difluoromethyl)phenyl]-2-[11-fluoro-5-methyl-6-oxo-9-[2-(trifluoromethyl)cyclopropyl]-5H-pyrido[2,3-d][1]benzazepin-7-yl]acetamide

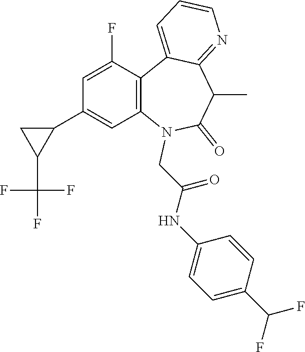 [SMILES: CC1C(=O)N(CC(=O)Nc2ccc(C(F)F)cc2)c2cc(C3CC3C(F)(F)F)cc(F)c2-c2cccnc21]

To a solution of 9-chloro-11-fluoro-5-methyl-5,7-dihydropyrido[2,3-d][1]benzazepin-6-one (20 mg, 0.072 mmol) NN217 and racemic 6-Methyl-2-[(1R,2R)-2-(trifluoromethyl)cyclopropyl]-1,3,6,2-dioxazaborocane-4,8-dione (CAS: 2735700-22-6, 30 mg, 0.11 mmol) in toluene (2.5 mL) were added Pd(OAc)2 (6 mg, 0.026 mmol), Ruphos (25 mg, 0.051 mmol) and finally a solution of K2CO₃ (63 mg, 0.456 mmol) in water (0.5 mL). The reaction mixture was flushed 3 times with argon and was heated in a sealed tube overnight at 120° C. The reaction mixture was filtered (SPE Filter) and evaporated under vacuum to give the crude compound as a dark oil. To the crude dark oil were added a solution of 2-chloro-N-[4-(difluoromethyl)phenyl]acetamide (7 mg, 0.032 mmol) in DMF (2 mL) followed by KI (5 mg, 0.03 mmol) and K2CO₃ (8 mg, 0.057 mmol) and the reaction mixture was stirred at room temperature overnight. The reaction mixture was filtered and directly purified by preparative HPLC (using 0.3% NH₃ in water/MeCN 30 to 100% as eluent) to give the title compound (5.1 mg, yield: 33%) as a solid. LC-MS m/z [M+H]⁺: 534.1; purity: 95.6%

### Example #347: 2-(3-chloro-5-cyclopropyl-10-methyl-9-oxo-4,8,12-triazatricyclo[9.4.0.0^{2,7}]pentadeca-1(15),2,4,6,11,13-hexaen-8-yl)-N-[4-(difluoromethyl)phenyl]acetamide

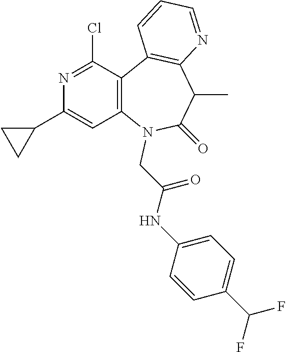 [SMILES: CC1C(=O)N(CC(=O)Nc2ccc(C(F)F)cc2)c2cc(C3CC3)nc(Cl)c2-c2cccnc21]

The title compound was prepared according to the general procedure 1 starting from 3-chloro-5-cyclopropyl-10-methyl-4,8,12-triazatricyclo[9.4.0.0^{2,7}]pentadeca-1(11),2(7), 3,5,12,14-hexaen-9-one NN166 (10 mg, 0.03 mmol) and 2-chloro-N-[4-(difluoromethyl)phenyl]acetamide (8.5 mg, 0.04 mmol). After complete conversion, water (10 mL) was added and the reaction mixture was extracted with EtOAc (3×10 mL). The combined organic extracts were dried over MgSO₄, filtered, and concentrated under vacuum. The residue was purified by preparative HPLC (Purification Method P_B) to afford the title compound (6.5 mg, yield: 40%) as a white solid. LC-MS m/z: [M+H]⁺: 483.6/485.2; purity: 95%. ¹H NMR (400 MHz, DMSO-d₆) δ 10.45 (s, 1H), 8.64 (dd, J=4.8, 1.7 Hz, 1H), 8.20 (dd, J=7.9, 1.7 Hz, 1H), 7.68 (d, J=8.4 Hz, 2H), 7.51 (d, J=8.4 Hz, 2H), 7.48 (s, 1H), 7.48-7.45 (m, 1H), 6.96 (t, J=56.1 Hz, 1H), 4.67-4.50 (m, 2H), 3.81 (q, J=6.6 Hz, 1H), 2.23-2.15 (m, 1H), 1.47 (d, J=6.6 Hz, 3H), 1.09-0.95 (m, 4H). ¹⁹F NMR (376 MHz, DMSO-d₆) δ−108.06 (d, J=56.1 Hz).

### Example #348: Enantiomer (10S) or (10R) of N-(4-chloro-3-fluoro-phenyl)-2-[5-(difluoromethyl)-14-fluoro-10-methyl-9-oxo-4,8,12-triazatricyclo[9.4.0.0^{2,7}]pentadeca-1(15),2,4,6,11,13-hexaen-8-yl]acetamide

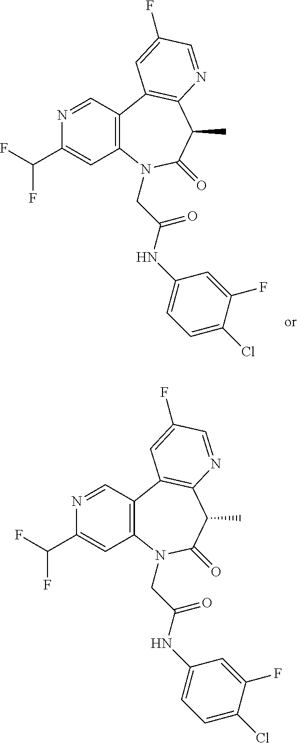 [SMILES: C[C@H]1C(=O)N(CC(=O)Nc2ccc(Cl)c(F)c2)c2cc(C(F)F)ncc2-c2cc(F)cnc21 ; C[C@@H]1C(=O)N(CC(=O)Nc2ccc(Cl)c(F)c2)c2cc(C(F)F)ncc2-c2cc(F)cnc21]

The title product was prepared according to the general procedure 1, starting from 5-(difluoromethyl)-14-fluoro-10-methyl-4,8,12-triazatricyclo[9.4.0.0^{2,7}]pentadeca-1(11),2,4,6,12,14-hexaen-9-one NN334 (51 mg, 0.165 mmol) and 2-chloro-N-(4-chloro-3-fluoro-phenyl)acetamide. After purification by reverse phase HPLC (acidic elution), the racemate was separated by Chiral SFC (column CHIRALPAK IG from Daicel, eluant 50% MeOH, 50% CO₂) to yield the pure enantiomer (19.8 mg, yield: 42%). LC-MS m/z [M+H]⁺: 479.1, 481.1; purity: 100%. ¹H NMR (400 MHz, DMSO-d₆) δ 10.64 (s, 1H), 9.02 (s, 1H), 8.75 (d, J=2.8 Hz, 1H), 8.31 (dd, J=9.4, 2.8 Hz, 1H), 7.81 (s, 1H), 7.72 (dd, J=11.9, 2.4 Hz, 1H), 7.52 (t, J=8.7 Hz, 1H), 7.32 (d, J=9.2 Hz, 1H), 7.08 (t, J=54.6 Hz, 1H), 4.65 (s, 2H), 3.72 (q, J=6.6 Hz, 1H), 1.49 (d, J=6.6 Hz, 3H). ¹⁹F NMR (376 MHz, DMSO-d₆) δ−114.59, −114.75, −115.55, −116.11, −116.91, −129.50. Chiral purity: 100%; rt=3.02 min (second eluting enantiomer). For information, first eluting enantiomer rt=1.29 min. Both measured by SFC, (CHIRALPAK IG from Daicel, 35% MeOH+0.1% NH₃, 65% CO₂).

### Example #349: N-(4-chloro-3-fluoro-phenyl)-2-(1-ethyl-3,7-dimethyl-2,6-dioxo-7H-pyrido[3,2-d][3]benzazepin-5-yl)acetamide

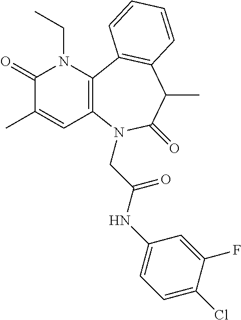 [SMILES: CCn1c2c(cc(C)c1=O)N(CC(=O)Nc1ccc(Cl)c(F)c1)C(=O)C(C)c1ccccc1-2]

### Step 1: Synthesis of ethyl 2-(3,7-dimethyl-2,6-dioxo-1-vinyl-7H-pyrido[3,2-d][3]benzazepin-5-yl)acetate #349_1

A stirring mixture of ethyl 2-(3,7-dimethyl-2,6-dioxo-1,7-dihydropyrido[3,2-d][3]benzazepin-5-yl)acetate N69_4 (100 mg, 0.279 mmol), copper(II) acetate (51 mg, 0.28 mmol), Na₂CO₃ (89 mg, 0.84 mmol), 2,2′-dipyridyl (44 mg, 0.28 mmol) and 4A MS (~0.5 g) in dry DCE (10 mL) was stirred at RT for 10 minutes. Potassium trifluoro(vinyl)boranide (94 mg, 0.70 mmol) was then added and the reaction mixture was heated at 60° C. for 18 h under air. The reaction mixture was cooled down to RT and filtered through a small pad of celite. The filter cake was rinsed with EtOAc (20 mL) and the filtrate was concentrated under vacuo. The crude was purified by column chromatography on silica gel (using a gradient of 0-10% MeOH in DCM as eluent) to afford the title compound as a light brown powder (76 mg, yield: 70%). LC-MS (Method 5) m/z [M+H]⁺: 367.2; rt: 1.77 min; purity: 100%. ¹H NMR (400 MHz, DMSO-d₆) δ 7.53 (d, J=1.3 Hz, 1H), 7.48-7.41 (m, 2H), 7.32 (t, J=7.7 Hz, 2H), 6.72 (dd, J=15.8, 8.5 Hz, 1H), 5.10 (d, J=8.4 Hz, 1H), 4.90 (d, J=15.8 Hz, 1H), 4.37 (d, J=17.3 Hz, 1H), 4.19 (d, J=17.3 Hz, 1H), 3.94 (qd, J=7.1, 5.0 Hz, 2H), 3.82 (q, J=6.7 Hz, 1H), 2.09 (d, J=1.1 Hz, 3H), 1.47 (d, J=6.8 Hz, 3H), 1.00 (t, J=7.1 Hz, 3H).

### Step 2: Synthesis of ethyl 2-(1-ethyl-3,7-dimethyl-2,6-dioxo-7H-pyrido[3,2-d][3]benzazepin-5-yl)acetate #349_2

Ethyl 2-(3,7-dimethyl-2,6-dioxo-1-vinyl-7H-pyrido[3,2-d][3]benzazepin-5-yl)acetate #349_1 (76 mg, 0.2 mmol) was dissolved in EtOH-MeOH 1:1 (4 mL) and Pd/C 5% (15 mg) was added. The resulting suspension was stirred at RT under 1 bar of hydrogen atmosphere for 1 h. The reaction mixture was filtered through a small pad of celite and the filter cake rinsed with MeOH (20 mL). The filtrate was concentrated under vacuo to afford the title compound as a cream coloured foam (65 mg, yield: 88%). LC-MS m/z [M+H]⁺: 369.2; purity: 99%. ¹H NMR (400 MHz, DMSO-d₆) δ 7.62 (dd, J=7.9, 1.4 Hz, 1H), 7.55 (td, J=7.6, 1.4 Hz, 1H), 7.49 (d, J=1.3 Hz, 1H), 7.44 (td, J=7.6, 1.3 Hz, 1H), 7.38 (d, J=7.9 Hz, 1H), 4.36 (d, J=17.3 Hz, 1H), 4.19-4.09 (m, 2H), 3.97-3.85 (m, 3H), 3.66 (q, J=6.7 Hz, 1H), 2.10 (d, J=1.1 Hz, 3H), 1.46 (d, J=6.8 Hz, 3H), 0.99 (td, J=7.0, 4.9 Hz, 6H).

### Step 3: Synthesis of N-(4-chloro-3-fluoro-phenyl)-2-(1-ethyl-3,7-dimethyl-2,6-dioxo-7H-pyrido[3,2-d][3]benzazepin-5-yl)acetamide #349

[2-(1,3,7-trimethyl-2,6-dioxo-7H-pyrido[3,2-d][3]benzazepin-5-yl)acetyl]oxylithium NN403 (66 mg, 0.2 mmol) prepared by saponification of #349_2 according to the procedure described for examples #68-#72 was dissolved in dry DMF (1.0 mL) to which HATU (97 mg, 0.26 mmol) was added followed by 4-bromo-3-chloro-aniline (48 mg, 0.23 mmol) and then DIPEA (0.10 mL, 0.58 mmol). The resulting mixture was stirred at RT over the weekend. The reaction mixture was quenched with water (20 mL) and extracted with EtOAc (3×50 mL). The combined organic extracts were washed with brine (3×50 mL), dried over MgSO₄, filtered and evaporated to give a brown gummy solid. The crude was purified by column chromatography on silica gel (using a gradient of 0-100% EtOAc in isohexane as eluent) to afford the title compound (24 mg, yield: 29%). LC-MS m/z [M+H]⁺: 468.2, 470.2; purity: 99%. ¹H NMR (400 MHz, DMSO-d₆) δ 10.40 (s, 1H), 7.72 (dd, J=11.9, 2.4 Hz, 1H), 7.64 (dd, J=7.6, 1.4 Hz, 1H), 7.59-7.54 (m, 2H), 7.51 (t, J=8.7 Hz, 1H), 7.45 (t, J=7.5 Hz, 1H), 7.39 (d, J=7.9 Hz, 1H), 7.30-7.26 (m, 1H), 4.30-4.11 (m, 3H), 3.90 (dq, J=13.7, 6.9 Hz, 1H), 3.67 (q, J=6.6 Hz, 1H), 2.10 (d, J=1.1 Hz, 3H), 1.46 (d, J=6.8 Hz, 3H), 1.00 (t, J=7.0 Hz, 3H).

### Example #350: N-(4-chloro-3-fluoro-phenyl)-2-(1,3-dimethyl-2,6-dioxo-7H-pyrido[3,2-d][3]benzazepin-5-yl)acetamide

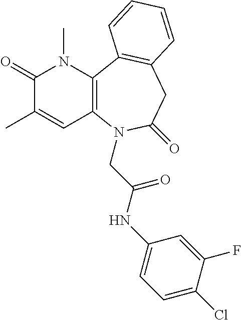 [SMILES: Cc1cc2c(n(C)c1=O)-c1ccccc1CC(=O)N2CC(=O)Nc1ccc(Cl)c(F)c1]

[2-(1,3-dimethyl-2,6-dioxo-7H-pyrido[3,2-d][3]benzazepin-5-yl)acetyl]oxylithium NN404 and 4-chloro-3-fluoro-aniline (45 mg, 0.31 mmol), N,N-diisopropylethylamine (0.13 mL, 0.78 mmol) and HATU (147 mg, 0.39 mmol) were sequentially added. The reaction mixture was stirred at RT for 18 h. The reaction mixture was diluted with EtOAc (50 mL) and washed with water (2×20 mL) and brine (3×50 mL). The organic phase was dried over Na₂SO₄, filtered and concentrated under vacuo. The crude was purified by column chromatography on silica gel (using a gradient of 0-10% MeOH in DCM as eluent) then by reverse phase column chromatography on C18 silica gel (using a gradient 30-70% acetonitrile in water with 0.1% formic acid as eluent) to afford the title compound as off-white solid (9.4 mg, yield: 8%). LC-MS m/z [M+H]⁺: 440.2, 442.2; purity: 99%. ¹H NMR (400 MHz, DMSO-d₆) δ 10.44 (s, 1H), 7.85-7.65 (m, 2H), 7.65-7.38 (m, 5H), 7.38-7.18 (m, 1H), 4.28 (d, J=16.6 Hz, 1H), 4.21 (d, J=16.6 Hz, 1H), 3.66 (d, J=12.3 Hz, 1H), 3.52 (d, J=12.4 Hz, 1H), 3.34 (s, 3H), 2.10 (d, J=1.1 Hz, 3H).

### Example #351: N-(4-chloro-3-fluoro-phenyl)-2-(2,7-dimethyl-1,6-dioxo-7H-pyrido[4,3-d][3]benzazepin-5-yl)acetamide

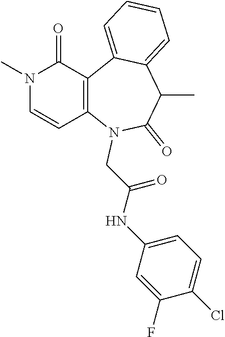 [SMILES: CC1C(=O)N(CC(=O)Nc2ccc(Cl)c(F)c2)c2ccn(C)c(=O)c2-c2ccccc21]

The title product was prepared according to the same procedure as for example #128, starting from [2-(2,7-dimethyl-1,6-dioxo-7H-pyrido[4,3-d][3]benzazepin-5-yl)acetyl]oxylithium NN418 (37 mg, 0.12 mmol) and 4-chloro-3-fluoro-aniline (CAS No: 367-22-6) (34 mg, 0.23 mmol). The crude was purified by column chromatography on silica gel (using a gradient of 0-100%+0.7 M NH₃ in MeOH in DCM as eluent) to afford the title compound as an off white solid (11 mg, yield: 22%). LC-MS m/z [M+H]⁺: 440.2, 442.2; purity: 98%. ¹H NMR (400 MHz, DMSO-d₆) δ 10.54 (s, 1H), 7.86 (dd, J=7.9, 1.4 Hz, 1H), 7.80-7.72 (m, 2H), 7.52 (t, J=8.7 Hz, 1H), 7.42 (td, J=7.5, 1.4 Hz, 1H), 7.32 (ddd, J=13.8, 7.3, 2.3 Hz, 3H), 6.38 (d, J=7.5 Hz, 1H), 4.45-4.29 (m, 2H), 3.50 (s, 3H), 3.42 (q, J=6.7 Hz, 1H), 1.46 (d, J=6.8 Hz, 3H). ¹⁹F NMR (376 MHz, DMSO-d₆) δ−114.65.

### Example #352: N-(4-chloro-3-fluoro-phenyl)-2-(9-chloro-2,3,7-trimethyl-1,6-dioxo-7H-pyrido[4,3-d][3]benzazepin-5-yl)acetamide

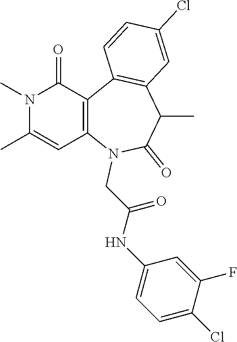 [SMILES: Cc1cc2c(c(=O)n1C)-c1ccc(Cl)cc1C(C)C(=O)N2CC(=O)Nc1ccc(Cl)c(F)c1]

The title product was prepared according to the same procedure as for example #128, starting from [2-(9-chloro-2,3,7-trimethyl-1,6-dioxo-7H-pyrido[4,3-d][3]benzazepin-5-yl)acetyl]oxylithium NN414 (9 mg, 0.025 mmol) and 4-chloro-3-fluoro-aniline (CAS No: 367-22-6) (4 mg, 0.027 mmol). The crude was purified by reverse phase chromatography on C18 silica gel (no buffer) to afford the title compound as an off white solid (2 mg, yield: 16%). LC-MS m/z [M+H]⁺: 488.1, 490.1; purity: 95%. ¹H NMR (400 MHz, CD30D) δ 7.88 (d, J=8.5 Hz, 1H), 7.71 (dd, J=11.5, 2.4 Hz, 1H), 7.44-7.35 (m, 2H), 7.34-7.31 (m, 1H), 7.29-7.25 (m, 1H), 6.50 (s, 1H), 4.49 (d, J=16.6 Hz, 1H), 4.34 (d, J=16.7 Hz, 1H), 3.64 (s, 3H), 3.55 (q, J=6.7 Hz, 1H), 2.52 (s, 3H), 1.57 (d, J=6.8 Hz, 3H). NH proton was not observed.

### Example #353: N-(4-chloro-3-fluoro-phenyl)-2-(7,12,14-trimethyl-8,13-dioxo-5,6,9,14-tetrazatricyclo[8.4.0.0^{2,6}]tetradeca-1(10),2,4,11-tetraen-9-yl)acetamide

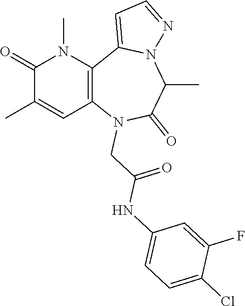 [SMILES: Cc1cc2c(n(C)c1=O)-c1ccnn1C(C)C(=O)N2CC(=O)Nc1ccc(Cl)c(F)c1]

### Step 1: Synthesis of 6-methoxy-5-methyl-2-(2-tetrahydropyran-2-ylpyrazol-3-yl)pyridin-3-amine #3531

Nitrogen was sparged through a solution of 2-bromo-6-methoxy-5-methyl-pyridin-3-amine NN285 (1.9 g, 8.3 mmol), 1-tetrahydropyran-2-yl-5-(4,4,5,5-tetramethyl-1,3,2-dioxaborolan-2-yl)pyrazole (3.2 mg, 11.6 mmol), K2CO₃ (3.4 mg, 25 mmol) and bis(di-tert-butyl(4-dimethylaminophenyl)phosphine)dichloropalladium(II) (236 mg, 0.33 mmol) in 1,4-dioxane (70 mL) and water (14 mL). The reaction mixture was heated at reflux for 3 h then cooled to RT. The solution was filtered on a pad of celite and washed with EtOAc (200 mL). The filtrate was concentrated under vacuum and the crude was purified by column chromatography on silica gel (using a gradient of 0-100% EtOAc in isohexane as eluent) to afford the title compound as a brown wax (2.3 g, yield: 85%). LC-MS m/z [M+H]⁺: 289.2; purity: 97%. ¹H NMR (400 MHz, DMSO-d₆) δ 7.57 (d, J=1.8 Hz, 1H), 7.08 (d, J=1.0 Hz, 1H), 6.53 (d, J=1.8 Hz, 1H), 5.86 (dd, J=10.0, 2.4 Hz, 1H), 4.67 (s, 2H), 3.89-3.81 (m, 1H), 3.79 (s, 3H), 3.44-3.34 (m, 1H), 2.38-2.24 (m, 1H), 2.10 (d, J=0.9 Hz, 3H), 1.97 (d, J=13.5 Hz, 1H), 1.88 (dd, J=13.3, 3.1 Hz, 1H), 1.66-1.41 (m, 3H).

### Step 2: Synthesis of 2-chloro-N-[6-methoxy-5-methyl-2-(2-tetrahydropyran-2-ylpyrazol-3-yl)-3-pyridyl]propenamide #353_2

To a suspension of 6-methoxy-5-methyl-2-(2-tetrahydropyran-2-ylpyrazol-3-yl)pyridin-3-amine #353_1 (2.3 g, 7.3 mmol) and K2CO₃ (5.0 g, 37 mmol) in dry DMF (70 mL) was added 2-chloropropanoyl chloride (1.4 mL, 14.6 mmol) dropwise at RT and the reaction mixture was stirred at RT overnight. A solution of 0.5 M LiCl (100 mL) was added and the mixture was extracted with EtOAc (3×70 mL). The combined extracts were washed with water (100 mL) and brine (100 mL), dried over Na₂SO₄, filtered and concentrated under vacuum. The crude was purified by column chromatography on silica gel (using a gradient of 0-80% EtOAc in isohexane as eluent) to afford the title compound as an orange solid (2.4 g, yield: 82%). LC-MS m/z [M+H]⁺: 379.0, 381.0; purity: 95%. ¹H NMR (400 MHz, DMSO-d₆) δ 9.79 (s, 1H), 7.68 (m, 1H), 7.56 (m, 1H), 6.44 (m, 1H), 5.87-5.71 (m, 1H), 4.64 (p, J=6.8 Hz, 1H), 3.92 (s, 3H), 3.84 (m, 1H), 3.49-3.37 (m, 1H), 2.28 (m, 1H), 2.20 (m, 3H), 1.91 (m, 2H), 1.64-1.44 (m, 6H). 1.3:1 mixture of diastereoisomers.

### Step 3: Synthesis of 2-chloro-N-[6-methoxy-5-methyl-2-(1H-pyrazol-5-yl)-3-pyridyl]propanamide hydrochloride #353_3

A suspension of 2-chloro-N-[6-methoxy-5-methyl-2-(2-tetrahydropyran-2-ylpyrazol-3-yl)-3-pyridyl]propanamide #353_2 (2.29 g, 5.74 mmol) and HCl (4 M in 1,4-dioxane, 14.4 mL, 57.4 mmol) in 1,4-dioxane (14 mL) was stirred at RT overnight. The precipitate was washed with TBME (100 mL) and dried under vacuo to afford the title compound as a white solid (1.4 g, yield: 70%). LC-MS m/z [M+H-HCl]⁺: 295.1, 297.1; purity: 80%. ¹H NMR (400 MHz, DMSO-d₆) δ 11.73 (s, 1H), 8.55 (s, 1H), 7.89 (d, J=2.3 Hz, 1H), 6.93 (d, J=2.3 Hz, 1H), 4.80 (q, J=6.8 Hz, 1H), 3.95 (s, 3H), 2.18 (d, J=0.8 Hz, 3H), 1.71 (d, J=6.8 Hz, 3H), two protons not visible.

### Step 4: Synthesis of 13-methoxy-7,12-dimethyl-5,6,9,14-tetrazatricyclo[8.4.0.0^{2,6}]tetradeca-1(14),2,4,10,12-pentaen-8-one #353_4

To a solution of 2-chloro-N-[6-methoxy-5-methyl-2-(1H-pyrazol-5-yl)-3-pyridyl]propenamide hydrochloride #353_3 (50 mg, 0.15 mmol) in DMF (2 mL) was added NaH (60%, 24 mg, 0.6 mmol). The reaction mixture was stirred at RT for 2.5 h then water (100 mL) was added followed by AcOH (3 mL). The resulting precipitate was collected by filtration, washed with water and dried under vacuo to afford the title compound as a white solid (1.1 g, yield: 91%). LC-MS m/z [M+H]⁺: 259.1; purity: 80%. ¹H NMR (400 MHz, DMSO-d₆) δ 10.41 (s, 1H), 7.59 (d, J=2.0 Hz, 1H), 7.39 (s, 1H), 6.75 (d, J=1.9 Hz, 1H), 4.85 (q, J=7.0 Hz, 1H), 3.93 (s, 3H), 2.20 (s, 3H), 1.56 (d, J=6.9 Hz, 3H).

### Step 5: Synthesis of ethyl 2-(7,12-dimethyl-8,13-dioxo-5,6,9,14-tetrazatricyclo[8.4.0.0^{2,6}]tetradeca-1(10),2,4,11-tetraen-9-yl)acetate #353_5

To a solution of ethyl 2-(13-methoxy-7,12-dimethyl-8-oxo-5,6,9,14-tetrazatricyclo[8.4.0.0^{2,6}]tetradeca-1(14),2,4,10,12-pentaen-9-yl)acetate NN386 prepared from #353_4 by the same procedure as the one described previously for examples N68_5 or N69_3 (1.15 g, 3.0 mmol) in MeCN (30 mL) was added KI (1.5 g, 9.0 mmol) and TMSCl (1.1 mL, 9.0 mmol). The reaction mixture was heated at 80° C. overnight then cooled to RT. The reaction mixture was concentrated under vacuo and the residue was partitioned between EtOAc (100 mL), saturated NaHCO₃ (50 mL) and water (50 mL). The layers were separated and the aqueous phase was extracted with EtOAc (2×100 mL). The combined extracts were washed with brine (100 mL), dried over Na₂SO₄, filtered and concentrated under vacuo. The crude was purified by column chromatography on silica gel (using a gradient of 0-20% MeOH in DCM as eluent) to afford the title compound as a beige solid (960 mg, yield: 95%). LC-MS m/z [M+H]⁺: 331.2; purity: 100%. ¹H NMR (400 MHz, DMSO-d₆) δ 11.95 (s, 1H), 7.59 (d, J=2.0 Hz, 1H), 7.55 (s, 1H), 6.77 (d, J=2.0 Hz, 1H), 4.91 (q, J=6.7 Hz, 1H), 4.47 (d, J=17.4 Hz, 1H), 4.32 (d, J=17.4 Hz, 1H), 3.99 (qd, J=7.2, 2.4 Hz, 2H), 2.08 (s, 3H), 1.68 (d, J=6.7 Hz, 3H), 1.05 (t, J=7.1 Hz, 3H).

### Step 6: Synthesis of ethyl 2-(7,12,14-trimethyl-8,13-dioxo-5,6,9,14-tetrazatricyclo[8.4.0.0^{2,6}]tetradeca-1(10),2,4,11-tetraen-9-yl)acetate #353_6

To a suspension of ethyl 2-(13-hydroxy-7,12-dimethyl-8-oxo-5,6,9,14-tetrazatricyclo[8.4.0.0^{2,6}]tetradeca-1(14),2,4,10,12-pentaen-9-yl)acetate #353_5 (0.96 g, 2.9 mmol) in MeCN (42 mL) was added Cs₂CO₃ (928 mg, 2.9 mmol). The reaction mixture was stirred for 10 minutes. Iodomethane (404 mg, 2.9 mmol) was added and the reaction mixture was stirred at RT overnight. The reaction mixture was concentrated under vacuo. The crude was purified by column chromatography on silica gel (using a gradient of 0-15% MeOH in DCM as eluent) to afford the title compound as a yellow wax (1.2 g, yield: 87%). LC-MS m/z [M+H]⁺: 345.2; purity: 74%. ¹H NMR (400 MHz, DMSO-d₆) δ 7.66 (d, J=2.0 Hz, 1H), 7.53 (d, J=1.3 Hz, 1H), 6.93 (d, J=2.0 Hz, 1H), 5.11-5.03 (m, 1H), 4.53-4.38 (m, 1H), 4.23 (d, J=17.4, 1H), 4.07-3.86 (m, 2H), 3.52 (s, 3H), 2.10 (d, J=1.1 Hz, 3H), 1.68 (d, J=6.8 Hz, 3H), 1.05 (t, J=7.1 Hz, 3H).

### Step 7: Synthesis of N-(4-chloro-3-fluoro-phenyl)-2-(7,12,14-trimethyl-8,13-dioxo-5,6,9,14-tetrazatricyclo[8.4.0.0²⁶]tetradeca-1(10),2,4,11-tetraen-9-yl)acetamide #353

To a suspension of lithium 2-(7,12,14-trimethyl-8,13-dioxo-5,6,9,14-tetrazatricyclo[8.4.0.0^{2,6}]tetradeca-1(10),2,4,11-tetraen-9-yl)acetate #353_6 NN405 prepared prepared by saponification of #353_5 according to the procedure described for examples #68-#72 (72.0%, 400 mg, 0.89 mmol) in DMF (4.4 mL) were added 4-chloro-3-fluoro-aniline (195 mg, 1.34 mmol), N,N-diisopropylethylamine (0.47 mL, 2.7 mmol) and HATU (680 mg, 1.8 mmol). The reaction mixture was stirred at RT for 3 days. A solution of 1 M LiCl (50 mL) was added and the suspension was extracted with EtOAc (3×40 mL). The combined organic extracts were washed with brine (100 mL), dried over Na₂SO₄, filtered and concentrated under reduced pressure. The crude was purified by column chromatography on silica gel (using a gradient of 0-10% MeOH in DCM as eluent) then by reverse phase column chromatography on C18 silica gel (using a gradient 5-80% MeCN in water+0.1% NH₄OH as eluent) to afford the title compound as an off-white solid (124 mg, yield: 31%). LC-MS m/z [M+H]⁺: 444.2, 446.2; purity: 100%. ¹H NMR (400 MHz, DMSO-d₆) δ 10.48 (s, 1H), 7.71 (dd, J=11.9, 2.4 Hz, 1H), 7.67 (d, J=2.0 Hz, 1H), 7.59 (s, 1H), 7.51 (t, J=8.7 Hz, 1H), 7.28 (d, J=9.5 Hz, 1H), 6.95 (d, J=2.0 Hz, 1H), 5.08 (q, J=6.6 Hz, 1H), 4.39 (d, J=16.6 Hz, 1H), 4.25 (d, J=16.6 Hz, 1H), 3.53 (s, 3H), 2.11 (s, 3H), 1.68 (d, J=6.7 Hz, 3H).

### Example #354: N-(4-chloro-3-fluoro-phenyl)-2-(9-fluoro-1,3,7-trimethyl-2,6-dioxo-7H-pyrido[3,2-d][3]benzazepin-5-yl)acetamide

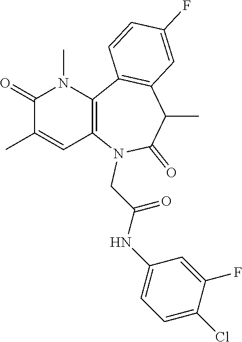 [SMILES: Cc1cc2c(n(C)c1=O)-c1ccc(F)cc1C(C)C(=O)N2CC(=O)Nc1ccc(Cl)c(F)c1]

The title product was prepared according to a procedure similar to the one described for example #128, starting from Intermediate NN420 (50 mg, 0.14 mmol) and 4-chloro-3-fluoroaniline. The crude was purified by column chromatography on silica gel (using a gradient of 0-100% EtOAc in isohexane as eluent) to afford the title compound as off white solid (40 mg, yield: 59%). LC-MS m/z [M+H]⁺: 472.1, 474.2; purity: 100%. ¹H NMR (400 MHz, DMSO-d₆) δ 10.42 (brs, 1H), 7.77 (dd, J=8.7, 5.7 Hz, 1H), 7.71 (dd, J=11.9, 2.4 Hz, 1H), 7.57 (d, J=1.3 Hz, 1H), 7.51 (t, J=8.7 Hz, 1H), 7.34-7.24 (m, 2H), 7.20 (dd, J=10.2, 2.6 Hz, 1H), 4.38-4.13 (m, 2H), 3.73 (q, J=6.7 Hz, 1H), 3.34 (s, 3H), 2.10 (d, J=1.1 Hz, 3H), 1.44 (d, J=6.7 Hz, 3H).

### Example #355: N-(4-chloro-3-fluoro-phenyl)-2-(1,2,7-trimethyl-3,6-dioxo-7H-pyrido[3,4-a][3]benzazepin-5-yl)acetamide

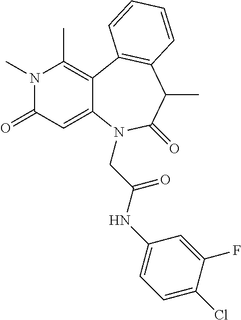 [SMILES: Cc1c2c(cc(=O)n1C)N(CC(=O)Nc1ccc(Cl)c(F)c1)C(=O)C(C)c1ccccc1-2]

### Step 1: Synthesis of 3,5-dibromo-2-methoxy-6-methyl-pyridin-4-amine #355_1

To a solution of 3-bromo-2-methoxy-6-methyl-pyridin-4-amine NN274 (500 mg, 2.30 mmol) in AcOH (3.0 mL) were added NaOAc (189 mg, 2.3 mmol) and bromine (0.21 mL, 4.0 mmol) at RT. The reaction mixture was then stirred at 80° C. for 3 h, then cooled to RT and stirred for 16 h. The reaction mixture was then diluted with water (20 mL) and extracted with EtOAc (3×20 mL). The combined organic extracts were washed with a saturated aqueous solution of Na₂CO₃ (30 mL), a saturated aqueous solution of Na₂S₂O₃ (30 mL) and brine (20 mL). The organic layer was then dried over MgSO₄, filtered and concentrated under vacuo. The crude was purified by column chromatography on silica gel (using a gradient of 0-100% EtOAc in isohexane as eluent) to afford the title compound as a yellow solid (501 mg, yield: 70%). LC-MS m/z [M+H]⁺: 294.9, 297.0, 298.9; purity: 98%. ¹H NMR (400 MHz, DMSO-d₆) δ 6.15 (bs, 2H), 3.82 (s, 3H), 2.39 (s, 3H).

### Step 2: Synthesis of 3-bromo-6-methoxy-2-methyl-pyridin-4-amine #355_2

To a solution of 3,5-dibromo-2-methoxy-6-methyl-pyridin-4-amine #355_1 (0.325 g, 1.03 mmol) in dry THF (15.0 mL) at −78° C. was added dropwise a solution of n-BuLi in hexanes (2.5 M, 0.434 mL, 1.08 mmol). The reaction mixture was stirred at −78° C. for 1 h and then a solution of n-BuLi in hexanes (2.5 M, 0.434 mL, 1.08 mmol) was added. The reaction mixture was stirred for 1 h and then warmed up to 0oC. The reaction mixture was quenched with quick addition of water (10 mL) and diluted with a saturated aqueous solution of NH₄Cl (25 mL). The aqueous phase was extracted with EtOAc (3×20 mL) and the combined organic layers were dried over Na₂SO₄, filtered and concentrated under vacuo. The residue was purified by column chromatography on silica gel (using a gradient of 0-100% EtOAc in isohexane as eluent) to afford the title compound as a brown oil (100 mg, yield: 43%). ¹H NMR (400 MHz, DMSO-de) δ 6.05 (s, 2H), 5.90 (s, 1H), 3.71 (s, 3H), 2.36 (s, 3H).

### Step 3: Synthesis of ethyl 2-(1,7-dimethyl-3,6-dioxo-2,7-dihydropyrido[3,4-a][3]benzazepin-5-yl)acetate #355_3

To a solution of ethyl 2-(3-methoxy-1,7-dimethyl-6-oxo-7H-pyrido[4,3-d][3]benzazepin-5-yl)acetate NN387 (prepared from NN338 according to the described alkylation procedure) (85%, 100 mg, 0.24 mmol) in MeCN (5 mL) was added KI (199 mg, 1.20 mmol) and TMSCl (0.152 mL, 1.20 mmol) at RT. NN338 was prepared by a Suzuki reaction according to described procedures starting from N64_2 and #355_2 described hereabove. The reaction mixture was then stirred at 80° C. for 16 h. The reaction mixture was then cooled to RT and concentrated under vacuo. The residue was then taken up in EtOAc (20 mL) and washed with water (20 mL) and a saturated aqueous solution of sodium thiosulphate (20 mL). The organic layer was then dried over MgSO₄, filtered and concentrated under vacuo to afford the title compound as a yellow oil (75 mg, yield: 89%). LC-MS m/z [M+H]⁺: 341.1; purity: 97%.

### Step 4: Synthesis of ethyl 2-(1,2,7-trimethyl-3,6-dioxo-7H-pyrido[3,4-a][3]benzazepin-5-yl)acetate #355_4

To a solution of ethyl 2-(1,7-dimethyl-3,6-dioxo-2,7-dihydropyrido[3,4-a][3]benzazepin-5-yl)acetate #355_3 (75.0 mg, 0.21 mmol) in MeCN (6 mL) were added Cs₂CO₃ (146 mg, 0.45 mmol) and a solution of iodomethane in MeCN (1 M, 0.43 mL, 0.43 mmol) at RT. The reaction mixture was then stirred at 80° C. for 16 hours. The reaction mixture was cooled to RT and concentrated under vacuo. The residue was then purified by column chromatography on silica gel (using a gradient of 0-10% MeOH in DCM as eluent) to afford the title compound as a yellow oil (79 mg, yield: 89%). LC-MS m/z [M+H]⁺: 355.2; purity: 93%. ¹H NMR (400 MHz, DMSO-d₆) δ 7.41-7.31 (m, 4H), 6.25 (s, 1H), 4.36 (s, 1H), 4.04-3.97 (m, 2H), 3.77 (q, J=6.7 Hz, 1H), 3.56 (s, 3H), 3.17 (d, J=5.3 Hz, 1H), 2.42 (s, 3H), 1.42 (d, J=6.8 Hz, 3H), 1.08 (t, J=7.1 Hz, 3H).

### Step 5: Synthesis of N-(4-chloro-3-fluoro-phenyl)-2-(1,2,7-trimethyl-3,6-dioxo-7H-pyrido[3,4-a][3]benzazepin-5-yl)acetamide #355

To a solution of 2-(1,2,7-trimethyl-3,6-dioxo-7H-pyrido[3,4-a][3]benzazepin-5-yl)acetic acid NN406 prepared by saponification of #355_4 according to procedures described for examples #68-#72 (85%, 70 mg, 0.18 mmol) in DMF (3 mL) were added 4-chloro-3-fluoro-aniline (0.023 mL, 0.21 mmol), HATU (139 mg, 0.37 mmol) and N,N-diisopropylethylamine (0.08 mL, 0.46 mmol) at RT. The reaction mixture was then stirred at RT for 16 h then diluted with EtOAc (20 mL), washed with water (10 mL), a solution of 1 M aqueous LiCl (10 mL) and brine (20 mL). The organic layer was then dried over MgSO₄, filtered and concentrated under vacuo. The crude was purified by column chromatography on silica gel (using a gradient of 0-100% EtOAc in isohexane as eluent) to afford the title compound as a white solid (53 mg, yield: 58%). LC-MS m/z [M+H]⁺: 454.1, 456.0; purity: 95%. ¹H NMR (400 MHz, DMSO-d₆) δ 10.49 (s, 1H), 7.73 (dd, J=11.9, 2.4 Hz, 1H), 7.51 (t, J=8.6 Hz, 1H), 7.45-7.23 (m, 5H), 6.30 (s, 1H), 4.51-4.28 (m, 2H), 3.79 (d, J=6.8 Hz, 1H), 3.56 (s, 3H), 2.43 (s, 3H), 1.43 (d, J=6.8 Hz, 3H).

### Example #356: N-(4-chloro-3-fluoro-phenyl)-2-(3-fluoro-5,10,13-trimethyl-9,12-dioxo-4,8,13-triazatricyclo[9.4.0.0^{2,7}]pentadeca-1(11),2,4,6,14-pentaen-8-yl)acetamide

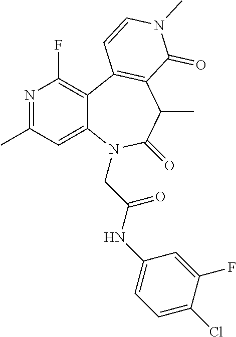 [SMILES: Cc1cc2c(c(F)n1)-c1ccn(C)c(=O)c1C(C)C(=O)N2CC(=O)Nc1ccc(Cl)c(F)c1]

### Step 1: Synthesis of 4-chloro-2-methoxy-3-[2-methoxyvinyl]pyridine #356_1

To a stirred suspension of (methoxymethyl)(triphenyl)phosphonium chloride (9.1 g, 27 mmol) in toluene (105 mL) at −10° C. was added a solution of KHMDS in THF (1.00 mol/L, 26.6 mL, 26.6 mmol) dropwise. The reaction mixture was stirred for 30 min at −10° C. then a solution of 4-chloro-2-methoxy-pyridine-3-carbaldehyde (CAS: 1008451-58-8) (60 g, 8.86 mmol) in toluene (17 mL) was added dropwise. The reaction mixture was warmed to RT and stirred for 2 h. The reaction mixture was quenched with a saturated aqueous solution of NH₄Cl (30 mL) and the layers were separated. The organic layer was dried over MgSO₄, filtered and concentrated under vacuo. The crude was purified by column chromatography on silica gel (using a gradient of 0-20% EtOAc in isohexane) to afford the title compound as a yellow oil that solidified upon standing (3.0 g, yield: 85%). LC-MS m/z [M+H]⁺: 200.0, 202.2; rt: 1.07 min and 1.16 min (mixture E/Z alkenes); purity: 50%.

### Step 2: Synthesis of 2-(4-chloro-2-methoxy-3-pyridyl)acetaldehyde #356_2

To a solution of 4-chloro-2-methoxy-3-[(E)-2-methoxyvinyl]pyridine #356_1 (50% purity, 3.00 g, 7.51 mmol) in MeCN (21.0 mL) was added an aqueous solution of HCl (6.00 mol/L, 21.3 mL, 128 mmol). The solution was stirred at RT for 16 hours. The reaction mixture was then neutralized by the addition of solid NaHCO₃, then diluted with water (20 mL). The resulting solution was then extracted with EtOAc (3×20 mL). The combined organic extracts were then passed through a hydrophobic frit and the solvent was removed under vacuo to afford the title compound as a colourless oil (2.5 g, yield: 81%). LC-MS m/z [M+H]⁺: 186.2, 188.0; rt: 0.93 min; purity: 45%.

### Step 3: Synthesis of 2-(4-chloro-2-methoxy-3-pyridyl)acetic acid #356_3

To a solution of 2-(4-chloro-2-methoxy-3-pyridyl)acetaldehyde #356_2 (45% purity, 2.50 g, 6.06 mmol) in tBuOH (20.0 mL) at 0° C. was added an aqueous solution of NaH₂PO₄ (2.00 mol/L, 9.09 mL, 18.2 mmol) and an aqueous solution of NaClO₂ (2.00 mol/L, 10.8 mL, 21.5 mmol). The reaction mixture was then stirred for 30 min at 0° C., then warmed to RT and stirred for 16 hours. An additional amount of aqueous NaH₂PO₄ (2.0 M, 9.09 mL, 18.2 mmol) and aqueous NaClO₂ (2.0 M, 10.8 mL, 21.5 mmol) were then added and the reaction mixture was stirred at RT for a further 16 h. The reaction mixture was then diluted with an aqueous solution of HCl 1 M (30 mL). The resulting solution was extracted with EtOAc (3×30 mL). The combined organic layers were washed with brine (10 mL), dried over Na₂SO₄, filtered and the solvent was removed under vacuo to afford the title compound as a yellow oil (2.79 g, yield: 91%). LC-MS m/z [M+H]⁺: 202.0, 204.0; purity: 40%.

### Step 4: Synthesis of ethyl 2-(4-chloro-2-hydroxy-3-pyridyl)acetate #356_4

To a solution of 2-(4-chloro-2-methoxy-3-pyridyl)acetic acid #356_3 (40%, 2.8 g, 5.5 mmol) in EtOH (40 mL) was added H₂SO₄ (271 mg, 2.8 mmol) at RT. The reaction mixture was then stirred at 80° C. for 16 hours. The reaction mixture was cooled to RT and concentrated under vacuo. The residue was partitioned between a saturated aqueous solution of NaHCO₃ (20 mL) and EtOAc (20 mL) and the layers were separated. The organic phase was filtered through a hydrophobic frit and concentrated under vacuo. The crude was purified by column chromatography on silica gel (using a gradient of 0-50% EtOAc in isohexane) to afford the title compound as a colourless oil (230 mg, yield: 18%). LC-MS m/z [M+H]⁺: 216.2; purity: 94%. ¹H NMR (400 MHz, DMSO-d₆) δ 11.93 (bs, 1H), 7.38 (d, J=7.1 Hz, 1H), 6.32 (d, J=7.1 Hz, 1H), 4.07 (q, J=7.1 Hz, 2H), 3.56 (s, 2H), 1.17 (t, J=7.1 Hz, 3H).

### Step 5: Synthesis of ethyl 2-(4-chloro-1-methyl-2-oxo-3-pyridyl)acetate #356_5

To a solution of ethyl 2-(4-chloro-2-hydroxy-3-pyridyl)acetate #356_4 (230 mg, 0.99 mmol) in MeCN (6.00 mL) was added Cs₂CO₃ (808 mg, 2.48 mmol) and a solution of iodomethane in MeCN (1 M, 2.98 mL, 2.98 mmol) at RT. The reaction mixture was then stirred at 80° C. for 4 h. An additional amount of iodomethane in MeCN (1.00 mol/L, 1.00 mL, 1.00 mmol) was added and the reaction mixture was stirred at 80° C. for a further 16 hours. The reaction mixture was cooled to RT and concentrated under vacuo. The crude was then purified by column chromatography on silica gel (using a gradient of 0-10% MeOH in DCM as eluent) to afford the title product as a yellow oil (150 mg, yield: 43%).LC-MS m/z [M+H]⁺: 230.2, 232.2; purity: 65%.

### Step 6: Synthesis of ethyl 2-(4-chloro-1-methyl-2-oxo-3-pyridyl)propanoate #356_6

To a solution of ethyl 2-(4-chloro-1-methyl-2-oxo-3-pyridyl)acetate #356_5 (60% purity, 200 mg, 0.523 mmol) in THF (10.0 mL) at 0° C. was added a solution of LiHMDS in THF (1 M, 0.60 mL, 0.60 mmol) dropwise and the reaction mixture was stirred for 30 minutes at 0° C. This was followed by the dropwise addition of Iodomethane (96.4 mg, 0.68 mmol) at 0° C. The reaction mixture was then warmed to RT and stirred for 16 h. The reaction mixture was then concentrated under vacuo. The crude was purified by column chromatography on silica gel (using a gradient of 0-100% EtOAc in isohexane as eluent) to afford the title compound as a light yellow oil (123 mg, yield: 68%). LC-MS m/z [M+H]⁺: 244.0, 246.2; purity: 70%.

### Step 7: Synthesis of N-(4-chloro-3-fluoro-phenyl)-2-(3-fluoro-5,10,13-trimethyl-9,12-dioxo-4,8,13-triazatricyclo[9.4.0.0²⁷]pentadeca-1(11),2,4,6,14-pentaen-8-yl)acetamide #356

The title product was prepared according to general procedure 1, starting from 3-fluoro-5,10,13-trimethyl-4,8,13-triazatricyclo[9.4.0.0^{2,7}]pentadeca-1(11),2,4,6,14-pentaene-9,12-dione NN339 prepared starting from #356_6 and N16_1 according to procedures described for the synthesis of lactams and 2-chloro-N-(4-chloro-3-fluoro-phenyl)acetamide (CAS 895641-02-8). Purification by column chromatography on silica gel (using a gradient of 0-10% MeOH in DCM as eluent) afforded the title compound as an off-white solid (68 mg, yield: 42%). LC-MS m/z [M+H]⁺: 473.0, 475.0; purity: 100%. ¹H NMR (400 MHz, DMSO-d₆) δ 10.63 (s, 1H), 7.80-7.73 (m, 1H), 7.67 (dd, J=7.2, 3.9 Hz, 1H), 7.55-7.51 (m, 1H), 7.36-7.31 (m, 1H), 7.26 (s, 1H), 6.48-6.45 (dd, J=7.2, 5.9 Hz, 1H), 4.80 (q, J=7.7 Hz, 1H), 4.70-4.46 (m, 2H), 3.49 (s, 3H), 2.47 (s, 3H), 0.84 (s, 3H).

### Example #357: Enantiomer (7S) or (7R) of 2-[3-(difluoromethyl)-1,7-dimethyl-2,6-dioxo-7H-pyrido[3,2-d][3]benzazepin-5-yl]-N-[4-(difluoromethyl)phenyl]acetamide

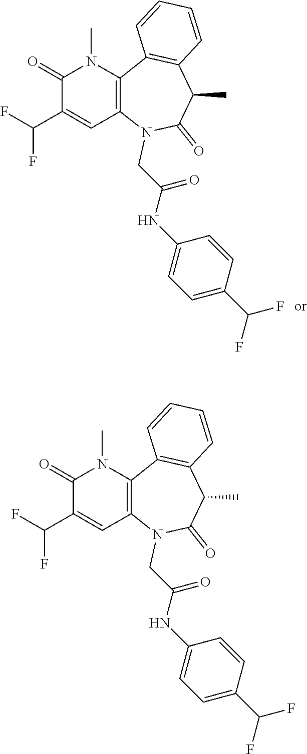 [SMILES: C[C@H]1C(=O)N(CC(=O)Nc2ccc(C(F)F)cc2)c2cc(C(F)F)c(=O)n(C)c2-c2ccccc21 ; C[C@@H]1C(=O)N(CC(=O)Nc2ccc(C(F)F)cc2)c2cc(C(F)F)c(=O)n(C)c2-c2ccccc21]

The title product was prepared according to a procedure similar to the one described for example #128 starting from [2-[3-(difluoromethyl)-1,7-dimethyl-2,6-dioxo-7H-pyrido[3,2-d][3]benzazepin-5-yl]acetyl]oxylithium NN412 (106 mg, 0.29 mmol) and 4-(difluoromethyl)aniline (49 mg, 0.35 mmol). After purification by reverse phase HPLC (acidic elution), the racemate was separated by Chiral SFC (Phenomenex Lux cellulose-4, eluent 50% MeCN-MeOH+0.1% NH₃, 50% CO₂) to yield the pure enantiomer as a light yellow solid (21.5 mg, 42%). LC-MS m/z [M+H]⁺: 488.2; purity: 96%. ¹H NMR (400 MHz, DMSO-d₆) δ 10.37 (s, 1H), 8.02 (s, 1H), 7.78 (d, J=7.9, 1.3 Hz, 1H), 7.68 (d, J=8.4 Hz, 2H), 7.62 (t, J=7.7, 1.4 Hz, 1H), 7.54-7.40 (m, 4H), 6.97 (t, 2H), 4.28 (dd, 2H), 3.82 (q, J=6.7 Hz, 1H), 3.39 (s, 3H), 1.46 (d, J=6.8 Hz, 3H). Chiral purity: 100%; rt=4.82 min (second eluting enantiomer). For information, first eluting enantiomer rt=1.18 min. (The column was a Phenomenex Lux cellulose-4, 40% MeCN-MeOH+0.1% NH₃, 60% CO₂)

### Example #358: Enantiomer (7S) or (7R) of N-[4-(difluoromethyl)phenyl]-2-[7,12,13-trimethyl-8,14-dioxo-5,6,9,13-tetrazatricyclo[8.4.0.0^{2,6}]tetradeca-1(10),2,4,11-tetraen-9-yl]acetamide

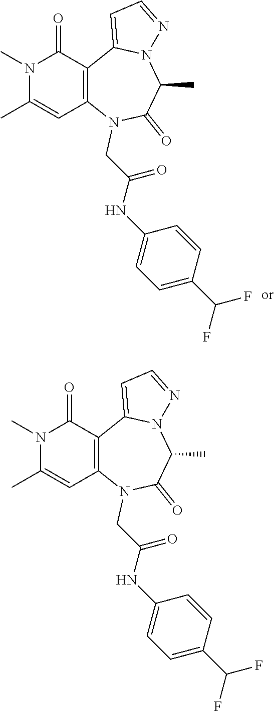 [SMILES: Cc1cc2c(c(=O)n1C)-c1ccnn1[C@@H](C)C(=O)N2CC(=O)Nc1ccc(C(F)F)cc1 ; Cc1cc2c(c(=O)n1C)-c1ccnn1[C@H](C)C(=O)N2CC(=O)Nc1ccc(C(F)F)cc1]

The title product was prepared according to a procedure similar to the one described for example #128 starting from lithium; 2-(7,12,13-trimethyl-8,14-dioxo-5,6,9,13-tetrazatricyclo[8.4.0.0^{2,6}]tetradeca-1(10),2,4,11-tetraen-9-yl)acetate NN413 (52% purity, 200 mg, 0.32 mmol) and 4-(difluoromethyl)aniline hydrochloride (CAS 1980063-54-4) (87 mg, 0.48 mmol). The crude was purified by column chromatography on silica gel (using a gradient of 0-20% MeOH in DCM as eluent) and then by reverse phase chromatography on C18 silica gel (basic elution). The racemate was separated by Chiral SFC (column CHIRALPAK IG from Daicel, eluant 45% MeOH+0.1% NH₃, 55% CO₂) to yield the pure enantiomer as a white solid (24 mg, yield: 27%). LC-MS m/z [M+H]⁺: 442.2; purity: 98%. ¹H NMR (400 MHz, DMSO-d₆) δ 10.46 (s, 1H), 7.70 (d, J=8.4 Hz, 2H), 7.58-7.48 (m, 3H), 6.97 (t, J=56.1 Hz, 1H), 6.75 (d, J=2.0 Hz, 1H), 6.37 (s, 1H), 4.79 (q, J=6.7 Hz, 1H), 4.51 (d, J=16.8 Hz, 1H), 4.45 (d, J=16.8 Hz, 1H), 3.50 (s, 3H), 2.43 (s, 3H), 1.70 (d, J=6.7 Hz, 3H). Chiral purity: 97.4%; rt=4.04 min (second eluting enantiomer). For information, first eluting enantiomer rt=1.99 min. Both measured by chiral SFC, column CHIRALPAK AY-H, from Daicel (35% MeOH+0.1% NH₃, CO₂ 65%).

### Example #359: Enantiomer (7S) or (7R) of N-[4-(difluoromethyl)phenyl]-2-[3,7-dimethyl-1,6-dioxo-7H-pyrano[4,3-d][3]benzazepin-5-yl]acetamide

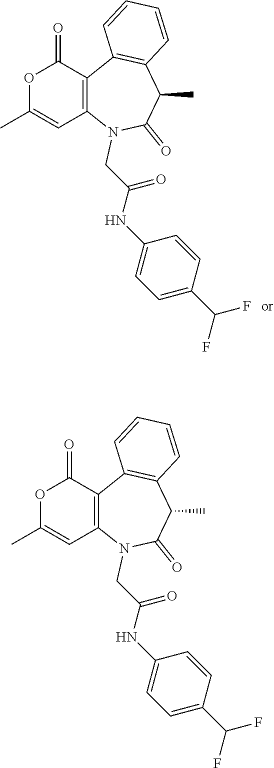 [SMILES: Cc1cc2c(c(=O)o1)-c1ccccc1[C@@H](C)C(=O)N2CC(=O)Nc1ccc(C(F)F)cc1 ; Cc1cc2c(c(=O)o1)-c1ccccc1[C@H](C)C(=O)N2CC(=O)Nc1ccc(C(F)F)cc1]

The title product was prepare according to the genera procedure 1, starting from 3,7-dimethyl-5,7-dihydropyrano[4,3-d][3]benzazepine-1,6-dione NN322 (100 mg, 0.384 mmol) and 2-chloro-N-[4-(difluoromethyl)phenyl]acetamide (CAS 872533-93-2). After purification by reverse phase HPLC (neutral elution), the racemate was separated by Chiral SFC (column Phenomenex A2, eluent 40% MeOH+0.1% NH₃, 60% CO₂) to yield the pure enantiomer as off white solid (23 mg, 30%). LC-MS m/z [M+H]⁺: 439.2; purity: 99%. ¹H NMR (400 MHz, DMSO-d₆) δ 10.47 (s, 1H), 7.78-7.66 (m, 3H), 7.56-7.44 (m, 3H), 7.40-7.27 (m, 2H), 6.97 (t, J=56.1 Hz, 1H), 6.48 (d, J=1.1 Hz, 1H), 4.43 (s, 2H), 3.55 (d, J=6.8 Hz, 1H), 2.30 (s, 3H), 1.48 (d, J=6.8 Hz, 3H). ¹⁹F NMR (376 MHz, DMSO-d₆) δ−108.09. Chiral purity: 100%; rt=4.09 min (second eluting enantiomer). For information, first eluting enantiomer rt=1.37 min. Both measured by Chiral SFC, Phenomenex A2, 40% MeOH+0.1% NH₃, 60% CO₂.

### Example #360: Enantiomer (10S) or (10R) of N-[4-(difluoromethyl)phenyl]-2-[5,10-dimethyl-3,9-dioxo-4-oxa-8,12-diazatricyclo[9.4.0.0^{2,7}]pentadeca-1(15),2(7),5,11,13-pentaen-8-yl]acetamide

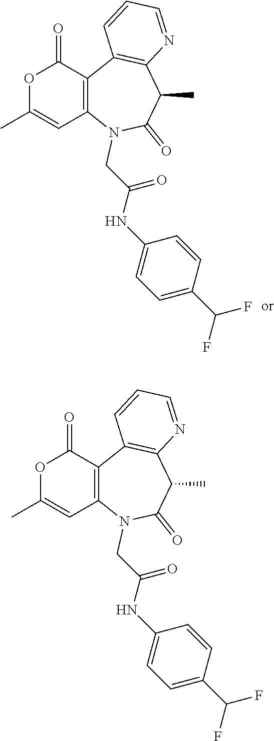 [SMILES: Cc1cc2c(c(=O)o1)-c1cccnc1[C@@H](C)C(=O)N2CC(=O)Nc1ccc(C(F)F)cc1 ; Cc1cc2c(c(=O)o1)-c1cccnc1[C@H](C)C(=O)N2CC(=O)Nc1ccc(C(F)F)cc1]

The title product was prepared according to the general procedure 1, starting from 5,10-dimethyl-4-oxa-8,12-diazatricyclo[9.4.0.0^{2,7}]pentadeca-1(11),2(7),5,12,14-pentaene-3,9-dione NN317 (92 mg, 0.36 mmol) and 2-chloro-N-[4-(difluoromethyl)phenyl]acetamide (95 mg, 0.41 mmol). After purification by reverse phase HPLC (acidic elution), the racemate was separated by Chiral HPLC (Column CHIRALPAK IH from Daicel, eluent 30% MeOH+0.03% NH₃, 70% CO₂) to yield the pure enantiomer as pale yellow solid (47 mg, 46%). LC-MS m/z [M+H]⁺: 440.2; purity: 99%. ¹H NMR (400 MHz, DMSO-d₆) δ 10.48 (s, 1H), 8.61 (dd, J=4.8, 1.7 Hz, 1H), 8.16 (dd, J=8.0, 1.7 Hz, 1H), 7.69 (d, J=8.4 Hz, 2H), 7.52 (d, J=8.3 Hz, 2H), 7.44 (dd, J=8.0, 4.7 Hz, 1H), 6.97 (t, J=56.1 Hz, 1H), 6.54 (s, 1H), 4.50 (d, J=18 Hz, 1H), 4.46 (d, J=18 Hz, 1H), 3.75 (q, J=6.6 Hz, 1H), 2.31 (s, 3H), 1.50 (d, J=6.6 Hz, 3H). Chiral purity: 98.4%; rt=1.49 min (first eluting enantiomer). For information, second eluting enantiomer rt=1.79 min. Both measured by Chiral SFC, CHIRALPAK IH from Daicel, 35% MeOH+0.03% NH₃, 65% CO₂.

### Example #361: N-[4-(difluoromethyl)phenyl]-2-(7,12,13-trimethyl-8,14-dioxo-3-thia-9,13-diazatricyclo[8.4.0.0^{2,6}]tetradeca-1(10),2(6),4,11-tetraen-9-yl)acetamide

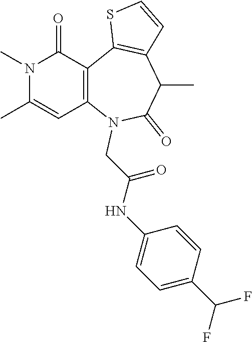 [SMILES: Cc1cc2c(c(=O)n1C)-c1sccc1C(C)C(=O)N2CC(=O)Nc1ccc(C(F)F)cc1]

The title product was prepared according to a procedure similar to the one described for example #128 starting from [2-(7,12,13-trimethyl-8,14-dioxo-3-thia-9,13-diazatricyclo[8.4.0.0^{2,6}]tetradeca-1(10),2(6),4,11-tetraen-9-yl)acetyl]oxylithium NN409 (28 mg, 0.082 mmol) and 4-(difluoromethyl)aniline hydrochloride. The crude was purified by column chromatography on silica gel (using a gradient of 0-100% EtOAc in isohexane as eluent) then by preparative HPLC (using a gradient 20-100% MeCN+0.1% NH₃ in water) to afford the title compound as a white solid (6 mg, yield: 14%). LC-MS m/z [M+H]⁺: 458.1; purity: 99%. ¹H NMR (400 MHz, DMSO-d₆) δ 10.43 (s, 1H), 7.76-7.66 (m, 3H), 7.52 (d, J=8.3 Hz, 2H), 7.11-6.83 (m, 2H), 6.36 (d, J=0.9 Hz, 1H), 4.51 (d, J=16.8 Hz, 1H), 4.35 (d, J=16.8 Hz, 1H), 3.52 (s, 3H), 3.06 (q, J=6.8 Hz, 1H), 2.42 (s, 3H), 1.52 (d, J=6.9 Hz, 3H).

### Example #362: N-[4-(difluoromethyl)phenyl]-2-(3-methoxy-2,7-dimethyl-1,6-dioxo-7H-pyrido[4,3-d][3]benzazepin-5-yl)acetamide

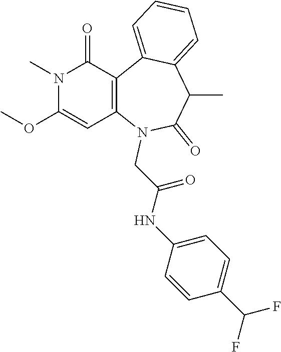 [SMILES: COc1cc2c(c(=O)n1C)-c1ccccc1C(C)C(=O)N2CC(=O)Nc1ccc(C(F)F)cc1]

The title product was prepared according to a procedure similar to the one described for example #128 starting from 2-(3-methoxy-2,7-dimethyl-1,6-dioxo-7H-pyrido[4,3-d][3]benzazepin-5-yl)acetic acid (83%, 60 mg, 0.15 mmol) NN410, 4-(difluoromethyl)aniline hydrochloride. The crude was purified by column chromatography on silica gel (using a gradient of 0-100% EtOAc in isohexane as eluent) to afford the title compound as an off-white solid (42 mg, 60%). LC-MS m/z [M+H]⁺: 468.0; purity: 97%. ¹H NMR (400 MHz, DMSO-d₆) δ 10.39 (s, 1H), 7.81 (dd, J=7.7, 1.5 Hz, 1H), 7.69 (d, J=8.4 Hz, 2H), 7.52 (d, J=8.3 Hz, 2H), 7.41-7.26 (m, 3H), 6.96 (s, 1H), 5.97 (s, 1H), 4.49-4.39 (m, 2H), 3.95 (s, 3H), 3.45 (d, J=6.9 Hz, 1H), 3.40 (s, 3H), 1.45 (d, J=6.8 Hz, 3H).

### Example #363: N-(4-chloro-3-fluoro-phenyl)-2-[3-(hydroxymethyl)-1,7-dimethyl-2,6-dioxo-7H-pyrido[3,2-d][3]benzazepin-5-yl]acetamide

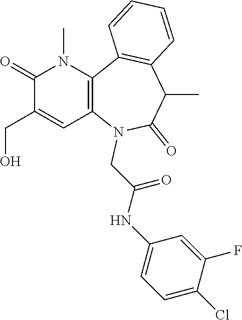 [SMILES: CC1C(=O)N(CC(=O)Nc2ccc(Cl)c(F)c2)c2cc(CO)c(=O)n(C)c2-c2ccccc21]

The title product was prepared according to a procedure similar to the one described for example #128 starting from [2-[3-(hydroxymethyl)-1,7-dimethyl-2,6-dioxo-7H-pyrido[3,2-d][3]benzazepin-5-yl]acetyl]oxylithium (23 mg, 0.0627 mmol) NN408, and 4-chloro-3-fluoro-aniline (18.3 mg, 0.125 mmol). The reaction mixture was diluted with water (80 mL) and extracted with EtOAc (3×10 mL). The combined organic extracts were washed with brine (20 mL), dried over anhydrous Na₂SO₄, filtered, and concentrated under vacuo. The crude was purified by column chromatography on silica gel (using a gradient of 0-10% MeOH in DCM as eluent) to afford the title compound as a pale yellow solid (2.7 mg, 9%). LC-MS m/z [M+H]⁺: 470.1; purity: 97%. ¹H NMR (400 MHz, DMSO-d₆) δ 10.45 (s, 1H), 7.77-7.66 (m, 3H), 7.62-7.49 (m, 2H), 7.47-7.37 (m, 2H), 7.30 (dd, J=9.0, 2.3 Hz, 1H), 5.22 (t, J=5.4 Hz, 1H), 4.40 (d, J=5.4 Hz, 2H), 4.22 (d, J=7.2 Hz, 2H), 3.71 (q, J=6.6 Hz, 1H), 3.35 (s, 3H), 1.47 (d, J=6.8 Hz, 3H). ¹⁹F NMR (376 MHz, DMSO-d₆) δ−114.64.

### Example #364: Enantiomer (7S) or (7R) of N-(4-chloro-3-fluoro-phenyl)-2-[-9-fluoro-2,7-dimethyl-1,6-dioxo-7H-pyrido[4,3-d][3]benzazepin-5-yl]acetamide

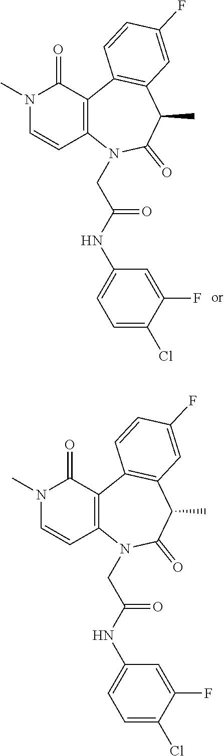 [SMILES: C[C@H]1C(=O)N(CC(=O)Nc2ccc(Cl)c(F)c2)c2ccn(C)c(=O)c2-c2ccc(F)cc21 ; C[C@@H]1C(=O)N(CC(=O)Nc2ccc(Cl)c(F)c2)c2ccn(C)c(=O)c2-c2ccc(F)cc21]

The title product was prepared according to a procedure similar to the one described for example #128 starting from [2-(9-fluoro-2,7-dimethyl-1,6-dioxo-7H-pyrido[4,3-d][3]benzazepin-5-yl)acetyl]oxylithium NN415 (85%, 111 mg, 0.28 mmol) and 4-chloro-3-fluoro-aniline (CAS No: 367-22-6). After purification by column chromatography on silica gel (using a gradient of 0-100% EtOH-EtOAc 3:1 in isohexane as eluent), the racemate was separated by Chiral SFC (column CHIRALPAK IG from Daicel, eluant 45% MeOH+0.1% NH₃, 55% CO₂) to yield the pure enantiomer as a white solid (6 mg, yield: 39%). LC-MS m/z [M+H]⁺: 458.1, 460.2; purity: 100%. ¹H NMR (400 MHz, DMSO-d₆) δ 10.50 (s, 1H), 8.41 (s, 1H), 7.87 (dd, J=7.7, 1.5 Hz, 1H), 7.73 (dd, J=11.8, 2.4 Hz, 1H), 7.62 (td, J=7.6, 1.5 Hz, 1H), 7.52 (d, J=8.9 Hz, 1H), 7.48 (d, J=7.4 Hz, 1H), 7.37 (d, J=7.8 Hz, 1H), 7.31-7.27 (m, 1H), 4.37 (s, 2H), 3.87 (q, J=6.7 Hz, 1H), 3.53 (s, 3H), 1.44 (d, J=6.8 Hz, 3H). Chiral purity: 98.5%; rt=2.86 min (second eluting enantiomer). For information, first eluting enantiomer rt=1.87 min. Both measured by chiral SFC, CHIRALPAK AY-H from Daicel, 45% MeOH+0.1% NH₃, 55% CO₂.

### Example #365: Enantiomer (10S) or (10R) of N-[4-(difluoromethyl)phenyl]-2-[5-(1-fluorocyclopropyl)-10-methyl-3,9-dioxo-4-oxa-8,12-diazatricyclo[9.4.0.0^{2,7}]pentadeca-1(15),2(7),5,11,13-pentaen-8-yl]acetamide

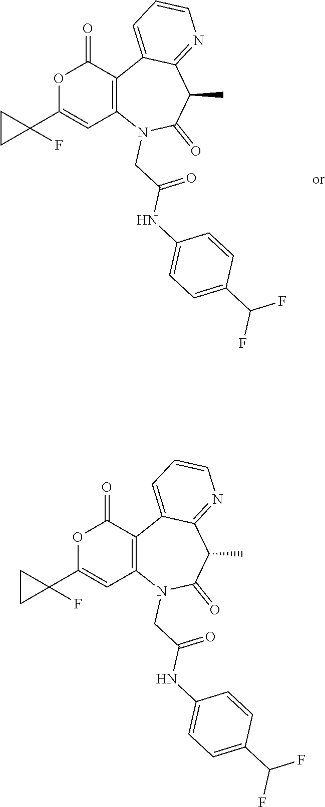 [SMILES: C[C@H]1C(=O)N(CC(=O)Nc2ccc(C(F)F)cc2)c2cc(C3(F)CC3)oc(=O)c2-c2cccnc21 ; C[C@@H]1C(=O)N(CC(=O)Nc2ccc(C(F)F)cc2)c2cc(C3(F)CC3)oc(=O)c2-c2cccnc21]

The title product was prepared according to the general procedure 1, starting from 5-(1-fluorocyclopropyl)-10-methyl-4-oxa-8,12-diazatricyclo[9.4.0.0^{2,7}]pentadeca-1(15),2(7),5,11,13-pentaene-3,9-dione NN329 (120 mg, 0.19 mmol) and 2-chloro-N-[4-(difluoromethyl)phenyl]acetamide (CAS 872533-93-2). After complete conversion, water was added to the reaction mixture and the resulting precipitate was collected by filtration and dried under vacuum. After purification by reverse phase HPLC (Method P_B), the racemate was separated by Chiral SFC (Column Phenomenex Lux 5 μM i-Cellulose-5, eluent 35% MeOH+0.03% NH₃, 65% CO₂) to yield the pure enantiomer as an off-white solid (2.3 mg, 21%). LC-MS m/z [M+H]⁺: 484.2; purity: 98%. ¹H NMR (400 MHz, DMSO-d₆) δ 10.55 (s, 1H), 8.63 (dd, J=4.7, 1.7 Hz, 1H), 8.17 (dd, J=8.0, 1.7 Hz, 1H), 7.69 (d, J=8.4 Hz, 2H), 7.52 (d, J=8.2 Hz, 2H), 7.45 (dd, J=8.0, 4.7 Hz, 1H), 6.97 (t, J=56.1 Hz, 1H), 6.78 (s, 1H), 4.56 (dd, 2H), 3.84 (q, J=6.5 Hz, 1H), 1.64-1.60 (m, 1H), 1.60-1.56 (m, 1H), 1.51 (d, J=6.7 Hz, 3H), 1.48-1.41 (m, 1H), 1.41-1.31 (m, 1H). Chiral purity: 97.8%; rt=2.072 min (second eluting enantiomer). For information, first eluting enantiomer rt=1.43 min. Both measured by Chiral SFC, Lux i-Cellulose-5 from Phenomenex, 35% MeOH+0.03% NH₃, 65% CO₂.

### Example #366: N-(4-chloro-3-fluoro-phenyl)-2-[4-(difluoromethyl)-10-methyl-3,9-dioxo-4,8,12-triazatricyclo[9.4.0.0^{2,7}]pentadeca-1(15),2(7),5,11,13-pentaen-8-yl]acetamide

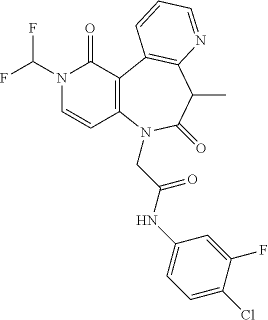 [SMILES: CC1C(=O)N(CC(=O)Nc2ccc(Cl)c(F)c2)c2ccn(C(F)F)c(=O)c2-c2cccnc21]

The title product was prepared according to a procedure similar to the one described for example #128 starting from [2-[4-(difluoromethyl)-10-methyl-3,9-dioxo-4,8,12-triazatricyclo[9.4.0.0^{2,7}]pentadeca-1(11),2(7),5,12,14-pentaen-8-yl]acetyl]oxylithium NN411 (30.0 mg, 0.085 mmol) and 4-chloro-3-fluoro-aniline. The resulting residue was purified by column chromatography on silica gel (using a gradient of 0-100% EtOAc in isohexane) to afford the title compound as an off-white solid (12 mg, 29%). LC-MS m/z [M+H]⁺: 477.2, 479.1; purity: 98%. ¹H NMR (400 MHz, DMSO-d₆) δ 10.61 (s, 1H), 8.62 (dd, J=4.8, 1.8 Hz, 1H), 8.22 (dd, J=8.0, 1.7 Hz, 1H), 7.94 (d, J=8.0 Hz, 1H), 7.92 (t, J=59.7 Hz, 1H), 7.73 (dd, J=11.8, 2.4 Hz, 1H), 7.52 (t, J=8.7 Hz, 1H), 7.43 (dd, J=8.0, 4.7 Hz, 1H), 7.31 (ddd, J=8.8, 2.4, 1.0 Hz, 1H), 6.63 (d, J=8.0 Hz, 1H), 4.55-4.41 (m, 2H), 3.73 (q, J=6.5 Hz, 1H), 1.50 (d, J=6.7 Hz, 3H). ¹⁹F NMR (376 MHz, DMSO-d₆) δ−102.06 (d, J=230.0 Hz), −103.51 (d, J=230.0 Hz), −114.59.

### Example #367: Enantiomer (8S) or (8R) of N-[4-(difluoromethyl)phenyl]-2-[8,13,14-trimethyl-9,15-dioxo-4,6,10,14-tetrazatricyclo[9.4.0.0^{2,7}]pentadeca-1(11),2(7),3,5,12-pentaen-10-yl]acetamide

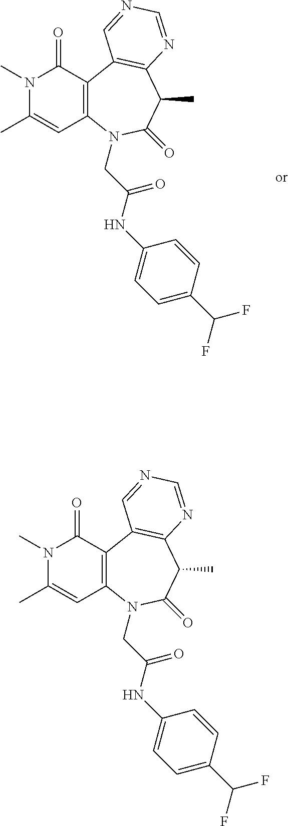 [SMILES: Cc1cc2c(c(=O)n1C)-c1cncnc1[C@@H](C)C(=O)N2CC(=O)Nc1ccc(C(F)F)cc1 ; Cc1cc2c(c(=O)n1C)-c1cncnc1[C@H](C)C(=O)N2CC(=O)Nc1ccc(C(F)F)cc1]

The title product was prepared according to a procedure similar to the one described for example #128 starting from [2-(8,13,14-trimethyl-9,15-dioxo-4,6,10,14-tetrazatricyclo[9.4.0.0^{2,7}]pentadeca-1(11),2(7),3,5,12-pentaen-10-yl)acetyl]oxylithium NN416 (42% purity, 352 mg, 0.44 mmol) and 4-(difluoromethyl)aniline hydrochloride. The crude was purified by column chromatography on silica gel (using a gradient EtOAc-EtOH 3:1 in heptane as eluent), the racemate was separated by Chiral SFC (column CHIRALPAK IG from Daicel, eluant 30% MeOH+0.1% NH₃, 70% CO₂) to yield the pure enantiomer as a white solid (21 mg, yield: 28%). LC-MS m/z [M+H]⁺: 454.2; purity: 99%. ¹H NMR (400 MHz, DMSO-d₆) δ 10.44 (s, 1H), 9.21 (s, 1H), 9.12 (s, 1H), 7.67 (d, J=8.4 Hz, 2H), 7.50 (d, J=8.4 Hz, 2H), 6.96 (t, J=56.1 Hz, 1H), 6.45 (s, 1H), 4.57-4.39 (m, 2H), 3.69 (d, J=6.7 Hz, 1H), 3.53 (s, 3H), 2.45 (s, 3H), 1.48 (d, J=6.6 Hz, 3H). ¹⁹F NMR (376 MHz, DMSO-d₆) δ−108.09. Chiral purity: 100%; rt=4.754 min (second eluting enantiomer). For information, first eluting enantiomer rt=2.383 min. Both measured by SFC, CHIRALPAK AY-H from Daicel, 30% MeOH+0.1% NH₃, CO₂ 70%.

### Example #368: Enantiomer (7S) or (7R) of N-[4-(difluoromethyl)phenyl]-2-[1,3,7-trimethyl-2,6-dioxo-7H-pyrido[4,3-a][3]benzazepin-5-yl]acetamide

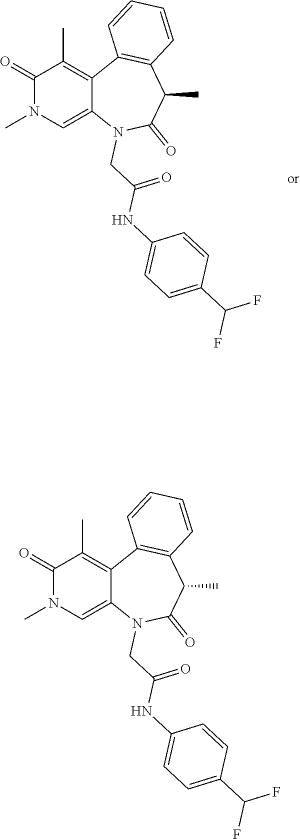 [SMILES: Cc1c2c(cn(C)c1=O)N(CC(=O)Nc1ccc(C(F)F)cc1)C(=O)[C@H](C)c1ccccc1-2 ; Cc1c2c(cn(C)c1=O)N(CC(=O)Nc1ccc(C(F)F)cc1)C(=O)[C@@H](C)c1ccccc1-2]

The title product was prepared according to a procedure similar to the one described for example #128 starting from lithium 2-(1,3,7-trimethyl-2,6-dioxo-7H-pyrido[3,4-d][3]benzazepin-5-yl)acetate NN417 (89% purity, 77 mg, 0.21 mmol) and 4-(difluoromethyl)aniline hydrochloride. The crude was purified by column chromatography. The racemate was separated by Chiral HPLC by (Column CHIRALPAK IH from Daicel, 30% MeOH+0.03% NH₃, 70% CO₂ to yield the pure enantiomer as a white solid (14 mg, 29%). LC-MS m/z [M+H]⁺: 452.2; purity: 97%. ¹H NMR (400 MHz, DMSO-d₆) δ 10.28 (s, 1H), 7.86 (s, 1H), 7.68 (d, J=8.4 Hz, 2H), 7.55-7.46 (m, 4H), 7.41 (td, J=7.4, 1.3 Hz, 1H), 7.35 (d, J=8.0 Hz, 1H), 6.96 (t, J=56.1 Hz, 1H), 4.33 (d, J=16.5 Hz, 1H), 4.27 (d, J=16.5 Hz, 1H), 3.78 (q, J=6.7 Hz, 1H), 3.52 (s, 3H), 2.07 (s, 3H), 1.40 (d, J=6.7 Hz, 3H). Chiral purity: 100%; rt=1.62 min (first eluting enantiomer). For information, second eluting enantiomer rt=3.33 min. Both measured by Chiral HPLC, CHIRALPAK IH from Daicel, 30% MeOH+0.03% NH₃, 70% CO₂.

### Example #369: Enantiomer (8S) or (8R) of N-(4-chloro-3-fluoro-phenyl)-2-[8,13-dimethyl-9-oxo-4,6,10,14-tetrazatricyclo[9.4.0.0^{2,7}]pentadeca-1(15),2,4,6,11,13-hexaen-10-yl]acetamide

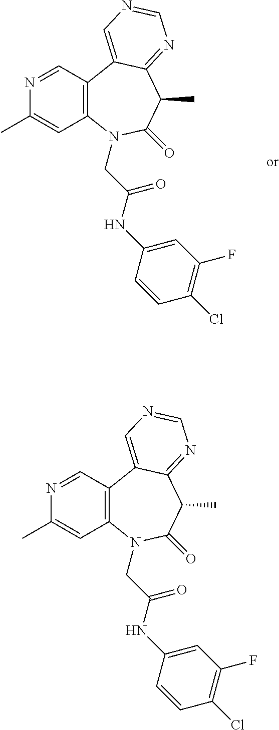 [SMILES: Cc1cc2c(cn1)-c1cncnc1[C@@H](C)C(=O)N2CC(=O)Nc1ccc(Cl)c(F)c1 ; Cc1cc2c(cn1)-c1cncnc1[C@H](C)C(=O)N2CC(=O)Nc1ccc(Cl)c(F)c1]

The title product was prepared according to the general procedure 1, starting from intermediate NN335 (38 mg, 0.16 mmol) and 2-chloro-N-(4-chloro-3-fluoro-phenyl)acetamide (CAS 895641-02-8). After complete conversion, the insoluble material was filtered off and the filtrate was concentrated under vacuum. After purification by reverse phase HPLC, the racemate was separated by chiral SFC with column Chiralcel OD 20 μm (50×266 mm) from Daicel, eluent CO₂+20% EtOH, at 360 mL/min, 300C and BPR 150 bar to yield the pure enantiomer as a white solid (49 mg, yield: 43%). Chiral purity: 95%; rt=3.86 min (second eluting enantiomer). For information, first eluting enantiomer rt=2.65 min. Both measured by chiral HPLC with column Chiralpak IGu (3×100 mm) from Daicel, eluent EtOH:n-heptane 1:1+0.1% DEA, at 1.5 mL/min and 300C. LC-MS m/z [M+H]⁺: 426.0; purity: 98%. ¹H NMR (400 MHz, DMSO-d₆) δ 10.58 (s, 1H), 9.27 (s, 1H), 9.17 (s, 1H), 8.84 (s, 1H), 7.72 (dd, J=11.8, 2.4 Hz, 1H), 7.51 (t, J=8.6 Hz, 1H), 7.42 (s, 1H), 7.34-7.27 (m, 1H), 4.66-4.52 (m, 2H), 3.77 (q, J=6.6 Hz, 1H), 2.58 (s, 3H), 1.48 (d, J=6.6 Hz, 3H).

### Example #370: N-[4-(difluoromethyl)phenyl]-2-(11-fluoro-5,9-dimethyl-6-oxo-5H-pyrimido[4,5-d][1]benzazepin-7-yl)acetamide

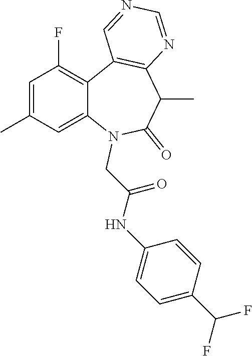 [SMILES: Cc1cc(F)c2c(c1)N(CC(=O)Nc1ccc(C(F)F)cc1)C(=O)C(C)c1ncncc1-2]

### Step 1: Synthesis of 3-fluoro-5-methyl-2-(4,4,5,5-tetramethyl-1,3,2-dioxaborolan-2-yl)aniline #370_1

To a solution of 3-fluoro-5-methyl-aniline (1 g, 7.99 mmol) in THF (8.00 mL) was added pinacolborane (1.02 g, 7.99 mol) and the reaction mixture was stirred at RT for 1 h. (1,5-cyclooctadiene)(methoxy)iridium(I) dimer (132 mg, 0.200 mmol) and 4,4′-di-tert-butyl-2,2′-bipyridine (94 mg, 0.40 mmol) were then added followed by additional pinacolborane (2.05 g, 16 mmol). The reaction mixture was heated at 80° C. overnight then cooled to RT. The reaction mixture was filtered through a pad of celite and washed with EtOAc (100 mL). The filtrate was concentrated under vacuum and the residue was purified by column chromatography on silica gel (using a gradient of 0-80% EtOAc in isohexane as eluent) to afford the title compound as an off-white solid (1.07 g, yield: 52%). ¹H NMR (400 MHz, DMSO-d₆) δ 6.21 (t, J=1.1 Hz, 1H), 6.01 (dt, J=11.0, 0.9 Hz, 1H), 5.75 (s, 2H), 2.13 (s, 3H), 1.27 (s, 12H); ¹⁹F NMR (376 MHz, DMSO-d₆) δ−101.86. Purity: 95%.

### Step 2: Synthesis of N-[4-(difluoromethyl)phenyl]-2-(11-fluoro-5,9-dimethyl-6-oxo-5H-pyrimido[4,5-d][1]benzazepin-7-yl)acetamide #370

The title product was prepared according to the general procedure 1, starting from 11-fluoro-5,9-dimethyl-5,7-dihydropyrimido[4,5-d][1]benzazepin-6-one NN340, prepared starting from #370_1 and N36_3 according to previously described procedures, (15.7 mg, 0.0598 mmol) and 2-chloro-N-[4-(difluoromethyl)phenyl]acetamide (CAS 872533-93-2). After purification by column chromatography on silica gel, the residue was triturated in MTBE to afford the title compound as off-white solid. LC-MS m/z [M+H]⁺: 441.2; purity: 100%. ¹H NMR (400 MHz, CD30D) δ 9.19 (s, 1H), 9.06 (d, J=4.4 Hz, 1H), 7.69 (d, J=8.3 Hz, 2H), 7.51 (d, J=8.4 Hz, 2H), 7.38 (s, 1H), 7.24-7.11 (m, 1H), 6.73 (t, J=56.4 Hz, 1H), 4.61 (d, J=16.5 Hz, 1H), 4.54 (d, J=16.6 Hz, 1H), 3.87 (q, J=6.6 Hz, 1H), 2.51 (s, 3H), 1.65 (d, J=6.7 Hz, 3H), NH not visible.

### Example #371: Enantiomer (5S) or (5R) of N-(4-chloro-3-fluoro-phenyl)-2-[11-fluoro-9-methoxy-5-methyl-6-oxo-5H-pyrimido[4,5-d][1]benzazepin-7-yl]acetamide

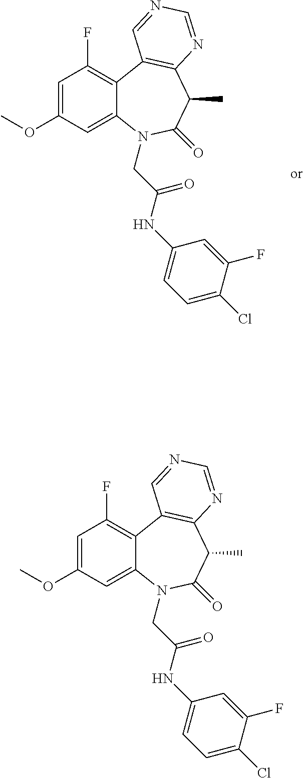 [SMILES: COc1cc(F)c2c(c1)N(CC(=O)Nc1ccc(Cl)c(F)c1)C(=O)[C@H](C)c1ncncc1-2 ; COc1cc(F)c2c(c1)N(CC(=O)Nc1ccc(Cl)c(F)c1)C(=O)[C@@H](C)c1ncncc1-2]

The title product was prepared according to the general procedure 1, starting from 11-fluoro-9-methoxy-5-methyl-5,7-dihydropyrimido[4,5-d][1]benzazepin-6-one NN341 (161 mg, 0.59 mmol) and 2-chloro-N-(4-chloro-3-fluoro-phenyl)acetamide (CAS 895641-02-8). After purification by column chromatography on silica gel, the racemate was separated by Chiral SFC (column CHIRALPAK IG from Daicel, eluant 35% MeOH, 65% CO₂) to yield the pure enantiomer (67 mg, yield: 35%). LC-MS m/z [M+H]⁺: 458.7, 460.9; purity: 100%. ¹H NMR (400 MHz, DMSO-d₆) δ 10.52 (s, 1H), 9.20 (s, 1H), 9.03 (d, J=4.4 Hz, 1H), 7.68 (dd, J=11.9, 2.4 Hz, 1H), 7.50 (t, J=8.7 Hz, 1H), 7.32-7.24 (m, 1H), 7.08 (d, J=10.5 Hz, 2H), 4.59 (d, J=16.6 Hz, 1H), 4.49 (d, J=16.6 Hz, 1H), 3.87 (s, 3H), 3.82 (q, J=6.5 Hz, 1H), 1.47 (d, J=6.6 Hz, 3H). Chiral purity: 100%; rt=3.02 min (second eluting enantiomer). For information, first eluting enantiomer rt=1.79 min. Both measured by HPLC, CHIRALPAK IG from Daicel, 45% MeOH+0.1% NH₃, 55% CO₂.

### Example #372: Enantiomer (5S) or (5R) of N-(4-chloro-3-fluoro-phenyl)-2-[9,11-difluoro-5-methyl-6-oxo-5H-pyrimido[4,5-d][1]benzazepin-7-yl]acetamide

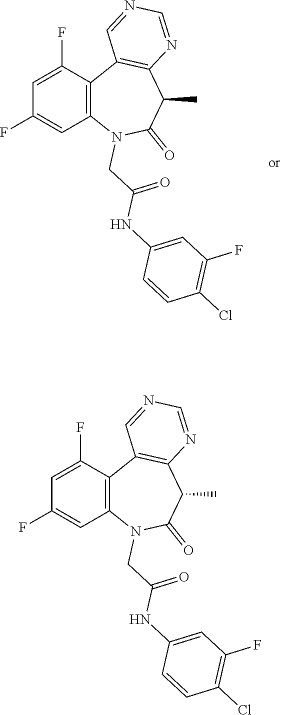 [SMILES: C[C@H]1C(=O)N(CC(=O)Nc2ccc(Cl)c(F)c2)c2cc(F)cc(F)c2-c2cncnc21 ; C[C@@H]1C(=O)N(CC(=O)Nc2ccc(Cl)c(F)c2)c2cc(F)cc(F)c2-c2cncnc21]

The title product was prepared according to the general procedure 1, starting from 9,11-difluoro-5-methyl-5,7-dihydropyrimido[4,5-d][1]benzazepin-6-one NN342 (160 mg, 0.52 mmol) and 2-chloro-N-(4-chloro-3-fluoro-phenyl)acetamide (CAS 895641-02-8). After purification by column chromatography on silica gel, the racemate was separated by Chiral SFC (column CHIRALPAK IG from Daicel, eluant 40% MeOH, 60% CO₂) to yield the pure enantiomer (16 mg, yield: 40%). LC-MS m/z [M+H]⁺: 447.1, 449.1; purity: 100%. ¹H NMR (400 MHz, DMSO-d₆) δ 10.60 (s, 1H), 9.25 (s, 1H), 9.09 (d, J=4.6 Hz, 1H), 7.69 (dd, J=11.9, 2.4 Hz, 1H), 7.55-7.46 (m, 2H), 7.43 (d, J=2.0 Hz, 1H), 7.29 (dd, 1H), 4.56 (dd, 2H), 3.88 (q, J=6.5 Hz, 1H), 1.47 (d, J=6.6 Hz, 3H).Chiral purity: 99.3%; rt=4.45 min (second eluting enantiomer). For information, first eluting enantiomer rt=1.54 min. Both measured by HPLC, A1 from Phenomenex, 35% MeOH+0.1% NH₃, 65% CO₂.

### Example #373: Enantiomer (5S) or (5R) of 2-[9-chloro-11-fluoro-3,5-dimethyl-6-oxo-5H-pyrimido[4,5-d][1]benzazepin-7-yl]-N-(4-chloro-3-fluoro-phenyl)acetamide

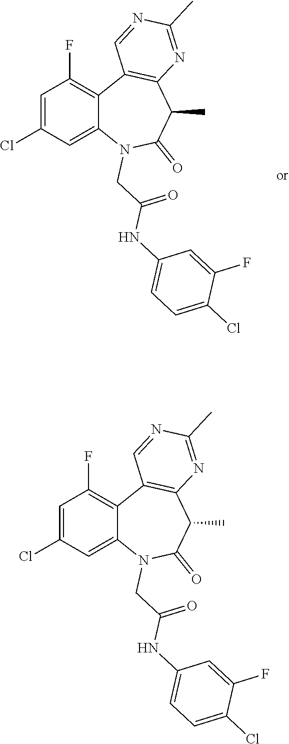 [SMILES: Cc1ncc2c(n1)[C@@H](C)C(=O)N(CC(=O)Nc1ccc(Cl)c(F)c1)c1cc(Cl)cc(F)c1-2 ; Cc1ncc2c(n1)[C@H](C)C(=O)N(CC(=O)Nc1ccc(Cl)c(F)c1)c1cc(Cl)cc(F)c1-2]

The title product was prepared according to the general procedure 1, starting from 9-chloro-11-fluoro-5-methyl-5,7-dihydropyridazino[4,3-d][1]benzazepin-6-one NN343 (91.0 mg, 0.295 mmol) and 2-chloro-N-(4-chloro-3-fluoro-phenyl)acetamide (CAS 895641-02-8). The racemate was separated by Chiral SFC (column CHIRALPAK IA from Daicel, eluant 35% MeOH+0.1% NH₃, 65% CO₂) to yield the pure enantiomer as a white solid (50 mg, yield: 36%). LC-MS m/z [M+H]⁺: 477.1, 479.1; purity: 96%. ¹H NMR (400 MHz, DMSO-d₆) δ 10.55 (s, 1H), 8.96 (d, J=4.5 Hz, 1H), 7.70 (dd, J=11.8, 2.4 Hz, 1H), 7.63 (dd, J=10.0, 2.0 Hz, 1H), 7.59 (t, J=1.7 Hz, 1H), 7.51 (t, J=8.7 Hz, 1H), 7.30 (dt, J=8.8, 1.6 Hz, 1H), 4.60 (d, J=16.8 Hz, 1H), 4.49 (d, J=16.8 Hz, 1H), 3.82 (q, J=6.6 Hz, 1H), 2.70 (s, 3H), 1.44 (d, J=6.6 Hz, 3H). ¹⁹F NMR (376 MHz, DMSO-d₆) δ−113.31, −114.63. Chiral purity: 99.7%; rt=2.63 min (second eluting enantiomer). For information, first eluting enantiomer rt=2.05 min. Both measured by SFC, CHIRALPAK IC from Daicel, 75% CO₂ and 25% MeOH+0.1% NH₃.

### Example #374: N-[4-(difluoromethyl)phenyl]-2-[(10R)-3-(2-hydroxyethoxy)-5,10-dimethyl-9-oxo-4,8,12-triazatricyclo[9.4.0.0^{2,7}]pentadeca-1(11),2(7),3,5,12,14-hexaen-8-yl]acetamide

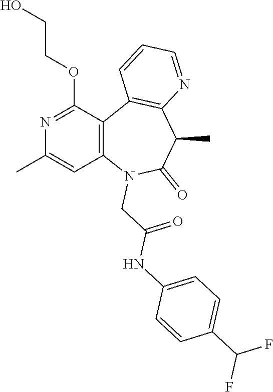 [SMILES: Cc1cc2c(c(OCCO)n1)-c1cccnc1[C@@H](C)C(=O)N2CC(=O)Nc1ccc(C(F)F)cc1]

N-[4-(difluoromethyl)phenyl]-2-[(10R)-(3-fluoro-5,10-dimethyl-9-oxo-4,8,12-triazatricyclo[9.4.0.0^{2,7}]pentadeca-1(11),2(7),3,5,12,14-hexaen-8-yl)acetamide #151 (100 mg, 0.23 mmol), was suspended in 2-methoxyethanol (60 equiv., 13.62 mmol) and HCl 4 N in dioxane (0.41 mmol, 0.1 mL) was added at rt. The heterogeneous white mixture turned homogenous and was heated at 90° C. for 2 h. The reaction mixture was diluted with EtOAc (40 mL) and washed with water (2×30 mL) and brine (50 mL). The organic phase was dried over sodium sulfate, filtered and concentrated under vacuum. The residue was purified by reverse phase HPLC to afford the title compound (20 mg, yield: 21%) as an off-white solid. LC-MS m/z [M+H]⁺: 483.4; purity: 99% ¹H NMR (400 MHz, DMSO-d₆) δ 10.43 (s, 1H), 8.58 (m, 1H), 8.28 (m, 1H), 7.69 (d, J=8.3 Hz, 2H), 7.51 (d, J 8.4 Hz, 2H), 7.41 (dd, J 7.9, 4.7 Hz, 1H), 7.02 (s, 1H), 6.97 (t, J 56.1 Hz, 1H), 4.81 (m, 1H), 4.62-4.38 (m, 3H), 4.21 (dt, J=10.7, 5.0 Hz, 1H), 3.71 (s, 2H), 3.63 (q, J 6.5 Hz, 1H), 2.44 (s, 3H), 1.48 (d, J 6.6 Hz, 3H).

### Example #375: N-(4-chloro-3-fluoro-phenyl)-2-[3-fluoro-5-(2-hydroxyethoxy)-10-methyl-9-oxo-4,8,12-triazatricyclo[9.4.0.0^{2,7}]pentadeca-1(15),2,4,6,11,13-hexaen-8-yl]acetamide

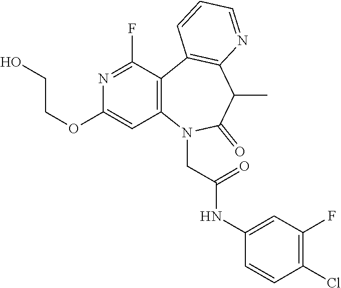 [SMILES: CC1C(=O)N(CC(=O)Nc2ccc(Cl)c(F)c2)c2cc(OCCO)nc(F)c2-c2cccnc21]

Sodium hydride (20 mg, 0.50 mmol) was added at room temperature to a solution of N-(4-chloro-3-fluoro-phenyl)-2-(3,5-difluoro-10-methyl-9-oxo-4,8,12-triazatricyclo[9.4.0.0^{2,7}]pentadeca-1(11),2(7),3,5,12,14-hexaen-8-yl)acetamide NN209a (100 mg, 0.22 mmol) and ethylene glycolumn (16 mg, 0.46 mmol) in THF (1 mL). The reaction mixture was stirred at 70° C. for 15 hours. The reaction mixture was diluted with EtOAc (25 mL) and the resulting mixture was washed with brine (25 mL), dried over MgSO₄, filtered and concentrated under vacuo to afford 108 mg of white solid. The crude was purified by reverse phase chromatography (Method G_B, 140 mLmin) to afford the title compound as a white solid (7.5 mg, yield: 7%), LC-MS m/z [M+H]⁺: 489.11; rt: 3.77 min; purity: 96%, LC-MS m/z [M+H]⁺: 489.11; rt: 4.14 min; purity: 94% and its isomer Example #376 as a white solid (17 mg, 16%), LC-MS m/z [M+H]⁺: 489.1; rt: 3.86 min; purity: 89%.

### Example #376: N-(4-chloro-3-fluoro-phenyl)-2-[5-fluoro-3-(2-hydroxyethoxy)-10-methyl-9-oxo-4.8,12-triazatricyclo[9.4.0.0^{2,7}]pentadeca-1(15),2,4,6,11,13-hexaen-8-yl]acetamide

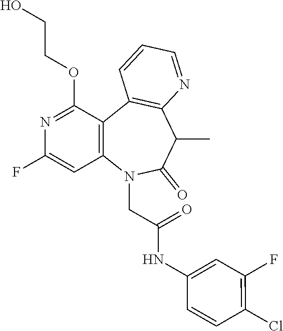 [SMILES: CC1C(=O)N(CC(=O)Nc2ccc(Cl)c(F)c2)c2cc(F)nc(OCCO)c2-c2cccnc21]

The title compound has been prepared following the procedure for Example #375 hereabove Example #378: N-[4-(difluoromethyl)phenyl]-2-[3-fluoro-10-methyl-5-(1-methylazetidin-3-yl)oxy-9-oxo-4,8,12-triazatricyclo[9.4.0.0^{2,7}]pentadeca-1(15),2,4,6,11,13-hexaen-8-yl]acetamide

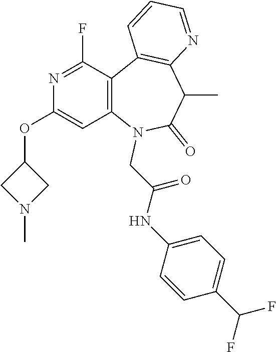 [SMILES: CC1C(=O)N(CC(=O)Nc2ccc(C(F)F)cc2)c2cc(OC3CN(C)C3)nc(F)c2-c2cccnc21]

To a solution of 1-methylazetidin-3-ol hydrochloride (172.0 mg, 1.35 mmol) in THF (4 mL) was added NaH (67.5 mg, 1.69 mmol) at RT. The reaction mixture was stirred for 10 min following by the addition of 2-(3,5-difluoro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide NN230 (300 mg, 0.68 mmol). The solution was stirred for 2 h 30 and then diluted with EtOAc (80 mL) and washed with water (120 mL). The aqueous phase was washed with EtOAc (50 mL). The combined organic extracts were washed three times with brine (100 mL), dried over Na₂SO₄, filtered and concentrated under vacuum. The residue was purified first by preparative HPLC (SFAR_C18 60 g cartridge) (0.1% TFA in water/MeCN (5 to 95%) as eluent) followed by a by preparative HPLC (Waters XBridge OBD MS C18 column using NH₄HCO₃ 10 mM+50 μl/L NH₄OH in water/MeCN (0 to 100%) as eluent) and then a preparative SFC chromatography (Daicel DCpak P4VP, CO₂ with MeOH from 5% to 50% as eluent) to give the titled compound (8 mg, Yield: 2.5%). LC-MS m/z [M+H]⁺: 512; purity: 98%.

### Example #379: 2-(3-chloro-10-hydroxy-5-methyl-9-oxo-4,8,12-triazatricyclo[9.4.0.0^{2,7}]pentadeca-1(15),2(7),3,5,11,13-hexaen-8-yl)-N-[4-(trifluoromethyl)phenyl]acetamide

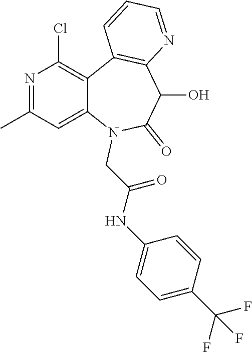 [SMILES: Cc1cc2c(c(Cl)n1)-c1cccnc1C(O)C(=O)N2CC(=O)Nc1ccc(C(F)(F)F)cc1]

A solution of 2-(3-fluoro-10-hydroxy-5-methyl-9-oxo-4,8,12-triazatricyclo[9.4.0.0^{2,7}]pentadeca-1(15),2(7),3,5,11,13-hexaen-8-yl)-N-[4-(trifluoromethyl)phenyl]acetamide #94 (140 mg, 0.30 mmol) in HCl (4 M in 1,4-dioxane, 48 mL, 30 mmol) was sealed and heated at 100° C. for 1.5 h. The reaction mixture was then allowed to cool to RT, poured into a saturated aqueous solution of NaHCO₃ (15 mL), and the resulting solution was stirred at RT for 10 minutes. The aqueous phase was extracted with EtOAc (3×10 mL), and then the combined organic extracts were washed with brine (2×10 mL), dried over MgSO₄, filtered and concentrated under vacuum. The crude product was then purified by preparative HPLC (acidic elution) to afford the title compound as an off-white solid (12 mg, yield: 8%). LC-MS m/z [M+H]⁺: 477.1, 479.1; purity: 100%. ¹H NMR (400 MHz, DMSO-d₆) δ 10.59 (s, 1H), 8.70 (dd, J=4.8, 1.6 Hz, 1H), 8.28 (dd, J=7.9, 1.6 Hz, 1H), 7.75 (d, J=8.6 Hz, 2H), 7.67 (d, J=8.8 Hz, 2H), 7.55 (dd, J=7.9, 4.8 Hz, 1H), 7.46 (s, 1H), 5.68 (d, J=8.7 Hz, 1H), 5.20 (d, J=8.7 Hz, 1H), 4.66-4.51 (m, 2H), 2.53 (s, 3H).

### Example #380: Enantiomer (7S) or (7R) of 2-[1,9-difluoro-7-hydroxy-3-methyl-6-oxo-7H-pyrido[4,3-d][3]benzazepin-5-yl]-N-[4-(difluoromethyl)phenyl]acetamide

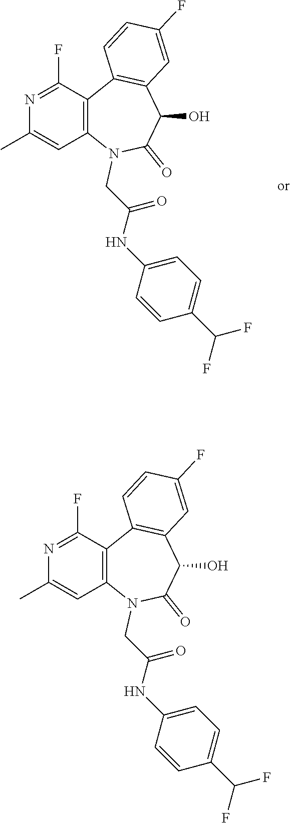 [SMILES: Cc1cc2c(c(F)n1)-c1ccc(F)cc1[C@@H](O)C(=O)N2CC(=O)Nc1ccc(C(F)F)cc1 ; Cc1cc2c(c(F)n1)-c1ccc(F)cc1[C@H](O)C(=O)N2CC(=O)Nc1ccc(C(F)F)cc1]

The title compound was prepared according to the general procedure 1, starting from 1,9-difluoro-7-hydroxy-3-methyl-5,7-dihydropyrido[4,3-d][3]benzazepin-6-one NN136 (133 mg, 0.43 mmol) and 2-chloro-N-[4-(difluoromethyl)phenyl]acetamide (141 mg, 0.64 mmol). After purification by preparative HPLC (Purification Method P_B), the racemate was separated by Chiral SFC (Chiralcel ODI from Daicel, CO₂+EtOH 20%) to afford the title compound as a white solid (26.7 mg, yield: 14%). LC-MS m/z: [M+H]⁺: 460.0; purity: 100%. LC-MS (Method B2) m/z: [M+H]⁺: 460.1; rt: 4.17 min; purity: 100%. ¹H NMR (400 MHz, DMSO-d₆) δ 10.52 (s, 1H), 7.77-7.67 (m, 3H), 7.53 (d, J=8.3 Hz, 2H), 7.41 (dd, J=9.7, 2.8 Hz, 1H), 7.35-7.28 (m, 2H), 6.98 (t, J=56.1 Hz, 1H), 6.31 (d, J=6.0 Hz, 1H), 5.01 (d, J=6.0 Hz, 1H), 4.65-4.50 (m, 2H), 2.49 (s, 3H). ¹⁹F NMR (376 MHz, DMSO-d₆) δ−70.22 (d, J=4.5 Hz), −108.09 (d, J=56.1 Hz), −111.52-−111.73 (m). Chiral purity: 100%; rt=1.95 min (first eluting enantiomer). For information, second eluting enantiomer rt=2.62 min. Both measured by HPLC (Chiralpak AD from Daicel, iPrOH 50%—heptane 50%—DEA 0.1%).

### Example #381: Enantiomer (5S) or (5R) of N-(4-chloro-3-fluoro-phenyl)-2-[9,11-difluoro-5-(hydroxymethyl)-6-oxo-5H-pyrido[2,3-d][1]benzazepin-7-yl]acetamide

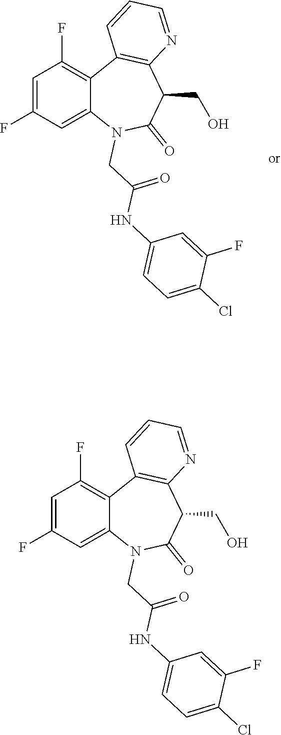 [SMILES: O=C(CN1C(=O)[C@H](CO)c2ncccc2-c2c(F)cc(F)cc21)Nc1ccc(Cl)c(F)c1 ; O=C(CN1C(=O)[C@@H](CO)c2ncccc2-c2c(F)cc(F)cc21)Nc1ccc(Cl)c(F)c1]

### Step 1: Synthesis of N-(4-chloro-3-fluoro-phenyl)-2-[9,11-difluoro-6-oxo-5-(2-trimethylsilylethoxymethyl)-5H-pyrido[2,3-d][1]benzazepin-7-yl]acetamide #381_1

The title product was prepared according to the general procedure 1, starting from 9,11-difluoro-5-(2-trimethylsilylethoxymethyl)-5,7-dihydropyrido[2,3-d][1]benzazepin-6-one NN345 (70 mg, 0.177 mmol) and 2-chloro-N-(4-chloro-3-fluoro-phenyl)acetamide (CAS 895641-02-8). The residue was then purified by column chromatography on silica gel to afford the title compound as a light yellow foam (53 mg, yield: 50%). LC-MS m/z [M+H]⁺: 562.2, 564.2; purity: 94%. ¹H NMR (400 MHz, DMSO-d₆) δ 10.52 (s, 1H), 8.62 (dd, J=4.8, 1.7 Hz, 1H), 8.10 (ddd, J=8.0, 4.5, 1.7 Hz, 1H), 7.69 (dd, J=11.9, 2.4 Hz, 1H), 7.52-7.40 (m, 4H), 7.26 (ddd, J=8.9, 2.5, 1.0 Hz, 1H), 4.62 (d, J=16.8 Hz, 1H), 4.49 (d, J=16.8 Hz, 1H), 4.27-4.15 (m, 2H), 3.70 (dd, J=7.4, 5.1 Hz, 1H), 3.52 (dd, J=8.7, 7.4 Hz, 2H), 0.81 (td, J=7.7, 1.5 Hz, 2H), −0.07 (s, 9H).

### Step 2: Synthesis of N-(4-chloro-3-fluoro-phenyl)-2-[rel-(5R)-9,11-difluoro-5-(hydroxymethyl)-6-oxo-5H-pyrido[2,3-d][1]benzazepin-7-yl]acetamide #381

A solution of N-(4-chloro-3-fluoro-phenyl)-2-[9,11-difluoro-6-oxo-5-(2-trimethylsilylethoxymethyl)-5H-pyrido[2,3-d][1]benzazepin-7-yl]acetamide #381_1 (53 mg, 0.088 mmol) in DCM (5 mL) and TFA (0.33 mL, 4.4 mmol) was stirred at RT for 3 h. The reaction mixture was then diluted with DCM (20 mL) and washed with a saturated aqueous solution of NaHCO₃ (2×20 mL). The organic phase was separated, dried over Na₂SO₄, filtered and concentrated under vacuo. After purification by column chromatography on silica gel (using a gradient of 0-100% EtOAc in isohexane as eluent), the racemate was separated by Chiral SFC (column Lux Cellulose-4 from Phenomenex, eluant 40% MeOH, 60% CO₂) to yield the pure enantiomer as a colorless solid (6 mg, yield: 29%). LC-MS m/z [M+H]⁺: 462.1, 464.1; 1.84 min; purity: 99%. ¹H NMR (400 MHz, DMSO-d₆) δ 10.54 (s, 1H), 8.63 (dd, J=4.8, 1.7 Hz, 1H), 8.14-8.06 (m, 1H), 7.69 (dd, J=11.9, 2.4 Hz, 1H), 7.53-7.39 (m, 4H), 7.31-7.25 (m, 1H), 4.61 (d, J=16.8 Hz, 1H), 4.53-4.46 (m, 2H), 4.32-4.21 (m, 2H), 3.65-3.59 (m, 1H). Chiral purity: 99.9%; rt=3.62 min (second eluting enantiomer). For information, first eluting enantiomer rt=1.93 min. Both measured by SFC, Lux Cellulose-4 from Phenomenex, 40% MeOH+0.1% NH₃, 60% CO₂.

### Example #382: Enantiomer (5S) or (5R) of 2-[9-chloro-11-fluoro-5-(hydroxymethyl)-6-oxo-5H-pyrido[2,3-d][1]benzazepin-7-yl]-N-(4-chloro-3-fluoro-phenyl)acetamide

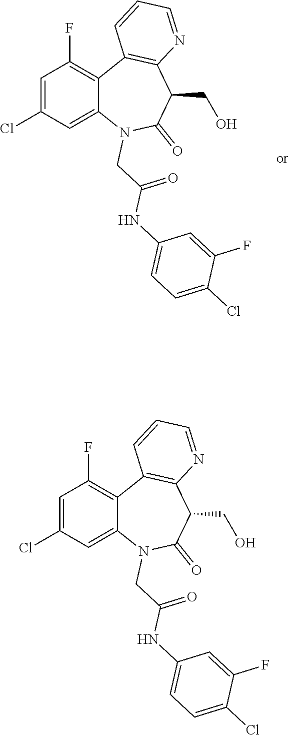 [SMILES: O=C(CN1C(=O)[C@H](CO)c2ncccc2-c2c(F)cc(Cl)cc21)Nc1ccc(Cl)c(F)c1 ; O=C(CN1C(=O)[C@@H](CO)c2ncccc2-c2c(F)cc(Cl)cc21)Nc1ccc(Cl)c(F)c1]

### Step 1: Synthesis of 2-[9-Chloro-11-fluoro-6-oxo-5-(2-trimethylsilylethoxymethyl)-5H-pyrido[2,3-d][1]benzazepin-7-yl]-N-(4-chloro-3-fluoro-phenyl)acetamide #382_1

The title product was prepared according to the general procedure 1, starting from 9-chloro-11-fluoro-5-(2-trimethylsilylethoxymethyl)-5,7-dihydropyrido[2,3-d][1]benzazepin-6-one NN346 (100 mg, 0.22 mmol) and 2-chloro-N-(4-chloro-3-fluoro-phenyl)acetamide (CAS 895641-02-8). After complete conversion the reaction mixture was quenched with water (10 mL), the solid was then collected by filtration, washed with water and dried to afford the title compound as a brown solid (122 mg, yield: 90%). LC-MS m/z [M+H]⁺: 578.2, 580.2; purity: 95%. ¹H NMR (400 MHz, DMSO-d₆) δ 10.54 (s, 1H), 8.63 (dd, J=4.8, 1.7 Hz, 1H), 8.11 (ddd, J=8.0, 4.5, 1.7 Hz, 1H), 7.70 (dd, J=11.9, 2.4 Hz, 1H), 7.64-7.58 (m, 2H), 7.54-7.47 (m, 2H), 7.27 (ddd, J=8.8, 2.4, 1.0 Hz, 1H), 4.62 (d, J=16.7 Hz, 1H), 4.49 (d, J=16.7 Hz, 1H), 4.26-4.13 (m, 2H), 3.70 (dd, J=7.5, 5.1 Hz, 1H), 3.52 (dd, J=8.6, 7.4 Hz, 2H), 0.81 (td, J=7.7, 1.6 Hz, 2H), −0.07 (s, 9H).

### Step 2: Synthesis of 2-[9-chloro-11-fluoro-5-(hydroxymethyl)-6-oxo-5H-pyrido[2,3-d][1]benzazepin-7-yl]-N-(4-chloro-3-fluoro-phenyl)acetamide #382

To a stirred solution of 2-[9-chloro-11-fluoro-6-oxo-5-(2-trimethylsilylethoxymethyl)-5H-pyrido[2,3-d][1]benzazepin-7-yl]-N-(4-chloro-3-fluoro-phenyl)acetamide #382_1 (100 mg, 0.16 mmol) in dichloromethane (10 mL) was added TFA (0.61 mL) and the resulting solution was stirred at ambient temperature for 2.5 h. The reaction mixture was diluted with DCM (20 mL) washed with sat. aqueous NaHCO₃ solution (2×20 mL) and dried over anhydrous Na₂SO₄ followed by purification by column chromatography on silica gel. The racemate was separated by Chiral SFC (column CHIRALPAK IA from Daicel, eluant 45% MeOH, 55% CO₂) to yield the pure enantiomer as a white solid (23 mg, yield: 34%). LC-MS m/z [M+H]⁺: 477.8, 479.9; purity: 99%. ¹H NMR (400 MHz, DMSO-d₆) δ 10.55 (s, 1H), 8.64 (dd, J=4.8, 1.7 Hz, 1H), 8.11 (ddd, J=7.9, 4.5, 1.7 Hz, 1H), 7.69 (dd, J=11.9, 2.4 Hz, 1H), 7.66-7.57 (m, 2H), 7.56-7.46 (m, 2H), 7.33-7.24 (m, 1H), 4.66-4.45 (m, 3H), 4.26 (qt, J=10.8, 6.0 Hz, 2H), 3.62 (dd, J=7.3, 5.5 Hz, 1H). Chiral purity: 100%; rt=3.51 min (second eluting enantiomer). For information, first eluting enantiomer rt=1.66 min.

Both measured by SFC, CHIRALPAK IA from Daicel, 50% MeOH+0.1% NH₃, 50% CO₂.

### Example #383: Enantiomer (5S) or (5R) of 2-[9-chloro-11-fluoro-5-methyl-6-oxo-5H-pyridazino[4,3-d][1]benzazepin-7-yl]-N-(4-chloro-3-fluoro-phenyl)acetamide

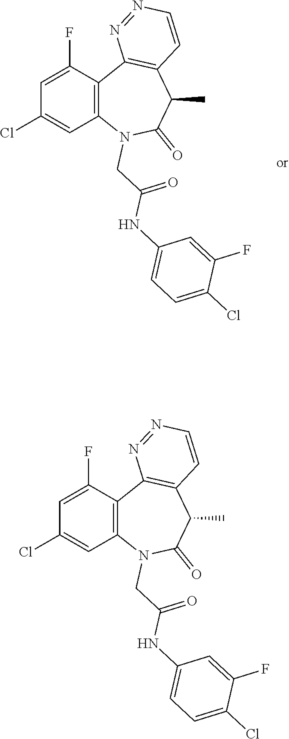 [SMILES: C[C@H]1C(=O)N(CC(=O)Nc2ccc(Cl)c(F)c2)c2cc(Cl)cc(F)c2-c2nnccc21 ; C[C@@H]1C(=O)N(CC(=O)Nc2ccc(Cl)c(F)c2)c2cc(Cl)cc(F)c2-c2nnccc21]

The title product was prepared according to the general procedure 1, starting from 9-chloro-11-fluoro-5-methyl-5,7-dihydropyridazino[4,3-d][1]benzazepin-6-one NN347 (91 mg, 0.29 mmol) and 2-chloro-N-(4-chloro-3-fluoro-phenyl)acetamide (CAS 895641-02-8). The racemate was separated by Chiral SFC (column CHIRALPAK IA from Daicel, eluant 45% MeOH 55% CO₂) to yield the pure enantiomer as a white solid (23 mg, yield: 34%). LC-MS m/z [M+H]⁺: 463.0, 465.1; purity: 99%. ¹H NMR (400 MHz, DMSO-d₆) δ 10.56 (s, 1H), 9.32 (d, J=5.4 Hz, 1H), 7.74 (dd, J=5.5, 1.0 Hz, 1H), 7.68 (dd, J=11.8, 2.4 Hz, 1H), 7.64 (dd, J=9.9, 2.0 Hz, 1H), 7.58 (t, J=1.7 Hz, 1H), 7.50 (t, J=8.7 Hz, 1H), 7.32-7.25 (m, 1H), 4.62 (d, J=16.8 Hz, 1H), 4.47 (d, J=16.7 Hz, 1H), 3.86-3.77 (m, 1H), 1.45 (d, J=6.6 Hz, 3H). ¹⁹F NMR (376 MHz, DMSO-d₆) δ−111.63, −114.64. Chiral purity: 100%; rt=3.51 min (second eluting enantiomer). For information, first eluting enantiomer rt=1.49 min. Both measured by SFC, CHIRALPAK IC from Daicel, 55% CO₂ and 45% MeOH+0.1% NH₃.

### Example #384: Enantiomer (5S) or (5R) of 2-[9-chloro-11-fluoro-5-methyl-6-oxo-5H-pyridazino[3,4-d][1]benzazepin-7-yl]-N-(4-chloro-3-fluoro-phenyl)acetamide

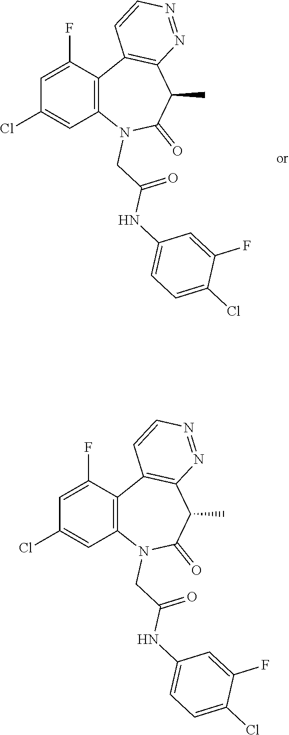 [SMILES: C[C@H]1C(=O)N(CC(=O)Nc2ccc(Cl)c(F)c2)c2cc(Cl)cc(F)c2-c2ccnnc21 ; C[C@@H]1C(=O)N(CC(=O)Nc2ccc(Cl)c(F)c2)c2cc(Cl)cc(F)c2-c2ccnnc21]

The title product was prepared according to the general procedure 1, starting from 9-chloro-11-fluoro-5-methyl-5,7-dihydropyridazino[3,4-d][1]benzazepin-6-one NN348 (130 mg, 0.431 mmol) and 2-chloro-N-(4-chloro-3-fluoro-phenyl)acetamide (CAS 895641-02-8). The racemate was separated by Chiral SFC (column CHIRALPAK IA from Daicel, eluant 50% MeOH 50% CO₂) to yield the pure enantiomer as an off white solid (37 mg, yield: 31%). LC-MS m/z [M+H]⁺: 463.0, 465.0; purity: 99%. ¹H NMR (400 MHz, DMSO-d₆) δ 10.55 (s, 1H), 9.36 (d, J=5.3 Hz, 1H), 8.00 (dd, J=5.3, 4.1 Hz, 1H), 7.72-7.60 (m, 3H), 7.50 (t, J=8.7 Hz, 1H), 7.32-7.25 (m, 1H), 4.64 (d, J=16.8 Hz, 1H), 4.49 (d, J=16.7 Hz, 1H), 4.05 (q, J=6.5 Hz, 1H), 1.61 (d, J=6.6 Hz, 3H). ¹⁹F NMR (376 MHz, DMSO-d₆) δ−112.82, −114.62. Chiral purity: 99.81%; rt=4.36 min (second eluting enantiomer). For information, first eluting enantiomer rt=2.53 min. Both measured by SFC, CHIRALPAK IC from Daicel, 45% MeOH+0.1% NH₃, 55% CO₂.

### Example #385: Enantiomer (5S) or (5R) of 2-[9-chloro-11-fluoro-5-methyl-6-oxo-5H-pyridazino[4,5-d][1]benzazepin-7-yl]-N-(4-chloro-3-fluoro-phenyl)acetamide

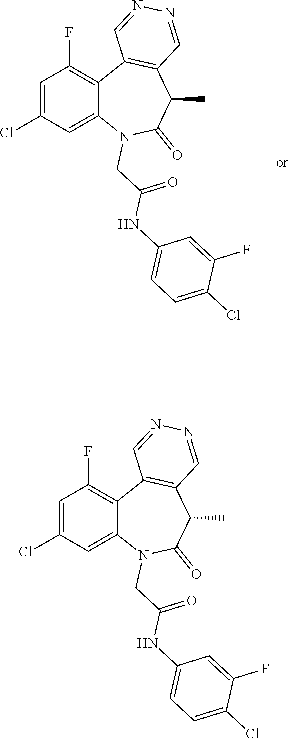 [SMILES: C[C@H]1C(=O)N(CC(=O)Nc2ccc(Cl)c(F)c2)c2cc(Cl)cc(F)c2-c2cnncc21 ; C[C@@H]1C(=O)N(CC(=O)Nc2ccc(Cl)c(F)c2)c2cc(Cl)cc(F)c2-c2cnncc21]

The title product was prepared according to the general procedure 1, starting from 9-chloro-11-fluoro-5-methyl-5,7-dihydropyridazino[4,5-d][1]benzazepin-6-one NN349 (361 mg, 0.12 mmol) and 2-chloro-N-(4-chloro-3-fluoro-phenyl)acetamide (CAS 895641-02-8). The racemate was separated by Chiral SFC (column CHIRALPAK IC from Daicel, eluant 50% MeOH 50% CO₂) to yield the pure enantiomer as a white solid (12 mg, yield: 21%). LC-MS m/z [M+H]⁺: 463.1, 465.0; purity: 100%. ¹H NMR (400 MHz, CD30D) δ 9.46 (d, J=4.6 Hz, 1H), 9.30 (s, 1H), 7.69-7.57 (m, 2H), 7.46 (dd, J=10.1, 1.9 Hz, 1H), 7.37 (t, J=8.5 Hz, 1H), 7.28-7.17 (m, 1H), 4.54 (s, 2H), 3.75 (q, J=6.8 Hz, 1H), 1.69 (d, J=6.8 Hz, 3H), one exchangeable proton not observed. Chiral purity: 99.1%; rt=4.26 min (second eluting enantiomer). For information, first eluting enantiomer rt=3.33 min. Both measured by SFC, CHIRALPAK IC from Daicel, 55% CO₂ and 45% MeOH+0.1% NH₃.

### Example #386: Enantiomer (7S) or (7R) of 2-[14-chloro-7,12-dimethyl-8-oxo-5,6,9,13-tetrazatricyclo[8.4.0.0^{2,6}]tetradeca-1(14),2,4,10,12-pentaen-9-yl]-N-[4-(difluoromethyl)phenyl]acetamide

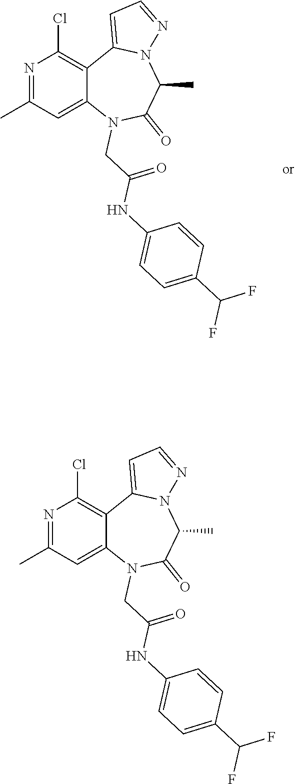 [SMILES: Cc1cc2c(c(Cl)n1)-c1ccnn1[C@@H](C)C(=O)N2CC(=O)Nc1ccc(C(F)F)cc1 ; Cc1cc2c(c(Cl)n1)-c1ccnn1[C@H](C)C(=O)N2CC(=O)Nc1ccc(C(F)F)cc1]

### Step 1: Synthesis of 2-chloro-6-methyl-3-(2-tetrahydropyran-2-ylpyrazol-3-yl)pyridin-4-amine #386_1

To a stirring solution of 3-bromo-2-chloro-6-methyl-pyridin-4-amine N30_1 (1.2 g, 5.4 mmol) in 1,4-dioxane (60 mL) was added 1-tetrahydropyran-2-yl-5-(4,4,5,5-tetramethyl-1,3,2-dioxaborolan-2-yl)pyrazole (CAS 903550-26-5) (2.4 g, 8.7 mmol), bis(di-tert-butyl(4-dimethylaminophenyl)phosphine)dichloropalladium(II) (307 mg, 0.43 mmol) and aqueous K2CO₃ (1.50 M, 8.67 mL, 13.0 mmol) at RT. The reaction mixture was purged with nitrogen for 5 minutes, then heated at 90° C. and stirred for 16 h. The reaction mixture was then cooled to RT and concentrated under vacuum. The crude was purified by column chromatography on silica gel (using a gradient of 0-100% EtOAc in isohexane as eluant) to afford the title compound as a yellow solid (860 mg, yield: 64%). ¹H NMR (400 MHz, DMSO-d₆) δ 7.64 (d, J=1.8 Hz, 1H), 6.51 (d, J=0.7 Hz, 1H), 6.30 (d, J=1.7 Hz, 1H), 5.78 (s, 2H), 4.95 (dd, J=9.7, 2.6 Hz, 1H), 3.81 (dt, J=13.5, 2.2 Hz, 1H), 3.40-3.34 (m, 1H), 2.38-2.29 (m, 1H), 2.27 (s, 3H), 1.98-1.91 (m, 1H), 1.83 (dd, J=13.3, 3.1 Hz, 1H), 1.63-1.41 (m, 3H). Only the major diastereoisomer assigned.

### Step 2: Synthesis of 13-chloro-7-(1-chloroethyl)-11-methyl-5,6,8,12-tetrazatricyclo[7.4.0.0^{2,6}]trideca-1(13),2,4,7,9,11-hexaene #386_2

To a stirring solution of 2-chloro-6-methyl-3-(2-tetrahydropyran-2-ylpyrazol-3-yl)pyridin-4-amine #386_1 (860 mg, 2.88 mmol) in DMF (20 mL) was added DIPEA (1.47 mL, 8.64 mmol) and 2-chloropropionyl chloride (0.84 mL, 8.64 mmol) sequentially. The reaction mixture was stirred at 100° C. for 18 h. The reaction mixture was allowed to cool to RT and then poured into brine (100 mL). The aqueous phase was extracted with EtOAc (3×50 mL). The combined organics were washed with aqueous LiCl (1 M, 50 mL) and brine (50 mL), dried over Na₂SO₄, filtered, and concentrated under vacuum onto silica gel. The crude was purified by column chromatography on silica gel (using a gradient of 0-100% EtOAc in isohexane as eluant) to afford the title compound as an off-white solid (530 mg, yield: 61%). ¹H NMR (400 MHz, DMSO-d₆) δ 8.47 (d, J=2.1 Hz, 1H), 7.82 (d, J=0.7 Hz, 1H), 7.64 (d, J=2.1 Hz, 1H), 6.14 (d, J=6.7 Hz, 1H), 2.63 (d, J=0.7 Hz, 3H), 2.01 (d, J=6.7 Hz, 3H).

### Step 3: Synthesis of 14-chloro-7,12-dimethyl-5,6,9,13-tetrazatricyclo[8.4.0.0^{2,6}]tetradeca-1(14),2,4,10,12-pentaen-8-one #386_3

To a stirring solution of 13-chloro-7-(1-chloroethyl)-11-methyl-5,6,8,12-tetrazatricyclo[7.4.0.0^{2,6}]trideca-1(13),2,4,7,9,11-hexaene #386_2 (0.53 g, 1.79 mmol) in 1,4-dioxane (20 mL) was added water (5.0 mL) and aqueous NaOH (2.0 M, 1.34 mL, 2.69 mmol). The resulting mixture was stirred at RT for 1 h. The reaction mixture was poured into saturated aqueous NH₄Cl solution (50 mL) and pH adjusted to −3-4 with concentrated HCl (~1 mL). The aqueous was extracted with EtOAc (3×50 mL) and the combined organics were dried with Na₂SO₄, filtered, and concentrated onto silica gel. The crude was purified by column chromatography on silica gel (using a gradient of 0-100% EtOAc in isohexane as eluant) to afford the title compound as a white solid (231 mg, yield: 47%). ¹H NMR (400 MHz, DMSO-d₆) δ 10.94 (s, 1H), 7.63 (d, J=2.0 Hz, 1H), 7.05 (s, 1H), 6.83 (d, J=2.1 Hz, 1H), 4.90 (s, 1H), 2.48 (s, 3H), 1.68 (s, 3H).

### Step 4: Synthesis of enantiomer (7S) or (7R) of 2-(14-chloro-7,12-dimethyl-8-oxo-5,6,9,13-tetrazatricyclo[8.4.0.0^{2,6}]tetradeca-1(14),2,4,10,12-pentaen-9-yl)-N-[4-(difluoromethyl)phenyl]acetamide #386

The title product was prepared according to the general procedure 1, starting from 14-chloro-7,12-dimethyl-5,6,9,13-tetrazatricyclo[8.4.0.0^{2,6}]tetradeca-1(14),2,4,10,12-pentaen-8-one #386_3 (230 mg, 0.630 mmol) and 2-chloro-N-[4-(difluoromethyl)phenyl]acetamide (CAS 872533-93-2). The crude product was purified by column chromatography on silica gel (using a gradient of 0-100% EtOAc in isohexane as eluant), followed by preparative HPLC (acidic elution). The racemate was then separated by chiral SFC (column CHIRALPAK IA from Daicel, eluant 60% MeOH+0.03% NH₃, 40% CO₂) affording the enantiomers, which were then further purified by preparative HPLC (acidic elution) to afford the pure enantiomer as an off-white solid (33 mg, yield: 12%). LC-MS m/z [M+H]⁺: 446.0, 448.0; purity: 99%. ¹H NMR (400 MHz, DMSO-d₆) δ 10.50 (s, 1H), 7.76-7.61 (m, 3H), 7.52 (d, J=8.3 Hz, 2H), 7.46 (s, 1H), 7.17-6.75 (m, 2H), 5.01 (d, J=6.7 Hz, 1H), 4.57 (s, 2H), 2.52 (s, 3H), 1.69 (d, J=6.6 Hz, 3H). Chiral purity: 100%; rt=3.27 min (second eluting enantiomer). For information, first eluting enantiomer rt=0.98 min. Both measured by SFC CHIRALPAK IA from Daicel, 55% MeOH+0.1% NH₃, 45% CO₂.

### Example #387: Enantiomer (7S) or (7R) of N-(4-chloro-3-fluoro-phenyl)-2-[14-chloro-12-methoxy-7-methyl-8-oxo-5,6,9,13-tetrazatricyclo[8.4.0.0^{2,6}]tetradeca-1(10),2,4,11,13-pentaen-9-yl]acetamide

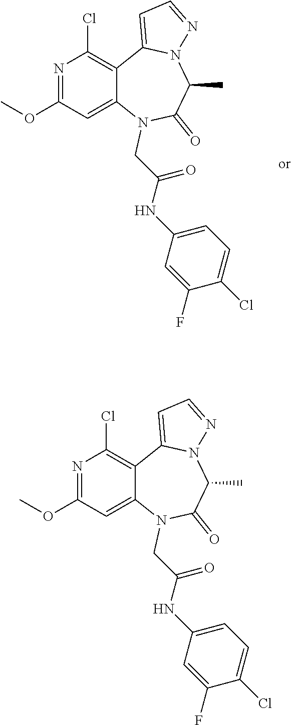 [SMILES: COc1cc2c(c(Cl)n1)-c1ccnn1[C@@H](C)C(=O)N2CC(=O)Nc1ccc(Cl)c(F)c1 ; COc1cc2c(c(Cl)n1)-c1ccnn1[C@H](C)C(=O)N2CC(=O)Nc1ccc(Cl)c(F)c1]

### Step 1: 3-bromo-2-chloro-5-iodo-6-methoxy-pyridin-4-amine #387_1

To a solution of 2-chloro-6-methoxy-pyridin-4-amine (2.16 g, 13.6 mmol) (CAS 1008304-85-5) in MeCN (40 mL) at 0° C. was added NIS (3.67 g, 16.3 mmol). The resulting mixture was stirred at RT for 3 h then NBS (2.91 g, 16.3 mmol) was added. The reaction mixture was stirred at RT overnight, then concentrated under reduced pressure. The crude was purified by column chromatography on silica gel (using a gradient of 0-50% EtOAc in isohexane as eluant) to afford the title compound as a pink solid (4.44 g, yield: 72%). LC-MS m/z [M+H]⁺: 363.0, 364.8; purity: 72%. ¹H NMR (400 MHz, DMSO-d₆) δ 6.40 (s, 2H), 3.80 (d, J=2.0 Hz, 3H).

### Step 2: Synthesis of 3-bromo-2-chloro-6-methoxy-pyridin-4-amine #387_2

To a solution of 3-bromo-2-chloro-5-iodo-6-methoxy-pyridin-4-amine #387_1 (4.44 g, 9.77 mmol) in THF (84 mL) at −40° C. (MeCN/dry ice bath) was added a solution of isopropylmagnesium chloride lithium chloride complex in THF (1.3 M, 6.6 mL, 11.4 mmol). The reaction mixture was stirred at −40° C. for 30 min and a solution of isopropylmagnesium chloride lithium chloride complex (1.3 M in THF) (6.6 mL, 11.4 mmol) was slowly added. The reaction mixture was stirred at −40° C. for 30 min. Three subsequent additions of isopropylmagnesium chloride lithium chloride complex (1.3 M in THF) (6.6 mL, 11.4 mmol) spaced 30 minutes apart were required to achieve full conversion. The reaction mixture was then stirred at −40° C. for 1 h. Saturated aqueous NH₄Cl solution (100 mL) was added and the aqueous phase was extracted with EtOAc (3×80 mL). The combined organic extracts were washed with brine (150 mL), dried over Na₂SO₄, filtered and concentrated under reduced pressure. The crude was purified by column chromatography on silica gel (using a gradient of 0% to 100% TBME in isohexane as eluent) to afford the title compound as an off-white solid (2.1 g, yield: 89%). LC-MS m/z [M+H]⁺: 237.0, 239.0; purity: 91%. ¹H NMR (400 MHz, DMSO-d₆) δ 6.52 (s, 2H), 6.00 (s, 1H), 3.72 (s, 3H).

### Step 3: Synthesis of 2-chloro-6-methoxy-3-(2-tetrahydropyran-2-ylpyrazol-3-yl)pyridin-4-amine #387_3

A suspension of 3-bromo-2-chloro-6-methoxy-pyridin-4-amine #387_2 (1.0 g, 4.1 mmol), 1-tetrahydropyran-2-yl-5-(4,4,5,5-tetramethyl-1,3,2-dioxaborolan-2-yl)pyrazole (CAS 903550-26-5) (1.4 g, 5.2 mmol), K2CO₃ (1.4 g, 10.3 mmol) and bis(di-tert-butyl(4-dimethylaminophenyl)phosphine)dichloropalladium(II) (146 mg, 0.21 mmol) in 1,4-dioxane (42 mL) and water (4.2 mL) was sparged with nitrogen for 5 minutes. The reaction mixture was then heated at 90° C. for 5 h then cooled to RT. The reaction mixture was filtered over a pad of celite and washed though with EtOAc (150 mL) then concentrated under reduced pressure. The crude was purified by column chromatography on silica gel (using a gradient of 0% to 10% MeOH in DCM as eluent) to afford the title compound as an orange wax (1.3 mg, yield: 75%). LC-MS m/z [M+H]⁺: 309.2; rt: 1.52 min and 1.63 min (as a mixture of diastereoisomers); purity: 72%.

### Step 4: Synthesis of 2-chloro-N-[2-chloro-6-methoxy-3-(2-tetrahydropyran-2-ylpyrazol-3-yl)-4-pyridyl]propenamide #387_4

To a solution of 2-chloro-6-methoxy-3-(2-tetrahydropyran-2-ylpyrazol-3-yl)pyridin-4-amine #387_3 (1.32 g, 3.12 mmol) in DMF (28 mL) at 0° C. was added NaH (60%, 150 mg, 3.75 mmol). The solution was stirred for 30 minutes then 2-chloropropanoyl chloride (0.42 mL, 4.4 mmol) was added dropwise. The reaction mixture was stirred for 1 h at 0° C. then allowed to warm to RT and stirred for 1 h. Water (100 mL) was added and the aqueous phase was extracted with EtOAc (3×50 mL). The combined organic extracts were washed with brine (100 mL), dried over Na₂SO₄, filtered and concentrated under reduced pressure. The crude was purified by column chromatography on silica gel (using a gradient of 0-100% EtOAc in isohexane as eluant) to afford the title compound as a yellow wax (695 mg, yield: 47%). LC-MS m/z [M+H]⁺: 399.0, 401.0; rt: 1.92 min and 1.99 (as a mixture of diastereoisomers); purity: 85%.

### Step 5: Synthesis of 13-chloro-7-(1-chloroethyl)-11-methoxy-5,6,8,12-tetrazatricyclo[7.4.0.0^{2,6}]trideca-1(13),2,4,7,9,11-hexaene #387_5

A solution of 2-chloro-N-[2-chloro-6-methoxy-3-(2-tetrahydropyran-2-ylpyrazol-3-yl)-4-pyridylpropenamide #387_4 (695 mg, 1.48 mmol) and HCl (4 M in 1,4-dioxane, 3.70 mL, 14.8 mmol) was stirred at RT overnight. The suspension was concentrated to dryness and the residue was purified by column chromatography on silica gel (using a gradient of 0-50% EtOAc in isohexane) to afford the title compound as an off-white solid (200 mg, yield: 45%). LC-MS m/z [M+H]⁺: 279.0, 299.0; purity: 99%. ¹H NMR (400 MHz, DMSO-d₆) δ 8.41 (d, J=2.1 Hz, 1H), 7.54 (d, J=2.0 Hz, 1H), 7.30 (s, 1H), 6.11 (q, J=6.7 Hz, 1H), 4.00 (s, 3H), 1.99 (d, J=6.7 Hz, 3H).

### Step 6: Synthesis of 14-chloro-12-methoxy-7-methyl-5,6,9,13-tetrazatricyclo[8.4.0.0²⁶]tetradeca-1(14),2,4,10,12-pentaen-8-one #387_6

To a suspension of 13-chloro-7-(1-chloroethyl)-11-methoxy-5,6,8,12-tetrazatricyclo[7.4.0.0^{2,6}]trideca-1(13),2,4,7,9,11-hexaene #387_5 (200 mg, 0.66 mmol) in DMSO (2.3 mL) was added aqueous NaOH (2 M, 0.66 mL, 1.3 mmol). The resulting orange solution was stirred at RT for 1 h, then water (20 mL) was added followed by AcOH (0.5 mL). The resulting suspension was filtered and the precipitate was washed with water and dried under vacuum to afford the title compound as an off-white solid (145 mg, yield: 76%). LC-MS m/z [M+H]⁺: 279.1; purity: 96%. ¹H NMR (400 MHz, DMSO-d₆) δ 10.87 (s, 1H), 7.61 (d, J=2.0 Hz, 1H), 6.78 (d, J=2.0 Hz, 1H), 6.60 (s, 1H), 4.93 (br s, 1H), 3.90 (s, 3H), 1.67 (br. S, 3H).

### Step 7: Synthesis of enantiomer (7S) or (7R) of N-(4-chloro-3-fluoro-phenyl)-2-(14-chloro-12-methoxy-7-methyl-8-oxo-5,6,9,13-tetrazatricyclo[8.4.0.0²⁶]tetradeca-1(14),2,4,10,12-pentaen-9-yl)acetamide #387

The title product was prepared according to the general procedure 1, starting from 14-chloro-12-methoxy-7-methyl-5,6,9,13-tetrazatricyclo[8.4.0.0^{2,6}]tetradeca-1(10),2,4,11,13-pentaen-8-one #387_6 (100 mg, 0.344 mmol) and 2-chloro-N-(4-chloro-3-fluoro-phenyl)acetamide (CAS 895641-02-8). Following complete conversion, water (20 mL) was added and the aqueous phase was extracted with EtOAc (3×20 mL). The combined organic extracts were washed with brine (2×20 mL), dried over with Na₂SO₄, filtered and concentrated under reduced pressure. Following purification by column chromatography on silica gel (using a gradient of 0-100% EtOAc in isohexane as eluant) the racemate was separated by chiral SFC (column Phenomenex IC, eluant 25% MeOH, 75% CO₂) affording the title compound as an off-white solid (45.0 mg, yield: 36%). LC-MS m/z [M+H]⁺: 463.9, 466.0; purity: 96%. ¹H NMR (400 MHz, DMSO-d₆) δ 10.59 (s, 1H), 7.78 (dd, J=11.9, 2.4 Hz, 1H), 7.53 (t, J=8.7 Hz, 1H), 7.37 (s, 1H), 7.34 (dd, J=9.1, 6.5 Hz, 2H), 7.27 (dd, J=7.8, 4.7 Hz, 1H), 6.92 (d, J=7.9 Hz, 1H), 5.26 (dd, J=9.2, 3.6 Hz, 1H), 4.66 (t, J=9.3 Hz, 1H), 4.60 (d, J=16.8 Hz, 1H), 4.50 (d, J=16.8 Hz, 1H), 4.19 (dd, J=9.3, 3.5 Hz, 1H), 2.48 (s, 3H). Chiral purity: 100%; rt=3.32 min (second eluting enantiomer). For information, first eluting enantiomer rt=1.905 min. Both measured by SFC CHIRALPAK IC from Daicel, 25% MeOH+0.1% NH₃, 75% CO₂.

### Example #388: 2-(11-chloro-9-cyano-5-methyl-6-oxo-5H-pyrazolo[1,5-d][1,4]benzodiazepin-7-yl)-N-[4-(difluoromethyl)phenyl]acetamide

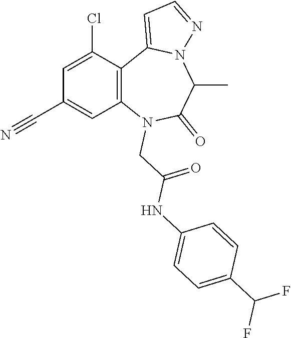 [SMILES: CC1C(=O)N(CC(=O)Nc2ccc(C(F)F)cc2)c2cc(C#N)cc(Cl)c2-c2ccnn21]

### Step 1: Synthesis of 3-amino-5-chloro-4-(2-tetrahydropyran-2-ylpyrazol-3-yl)benzonitrile #388_1

To a degassed mixture of 3-amino-4-bromo-5-chloro-benzonitrile (CAS 2383354-03-6) (500 mg, 2.16 mmol), 1-tetrahydropyran-2-yl-5-(4,4,5,5-tetramethyl-1,3,2-dioxaborolan-2-yl)pyrazole (CAS 903550-26-5) (0.93 g, 3.0 mmol) and CsF (984 mg, 6.5 mmol) in 1,4-dioxane (30 mL) and water (5 mL) was added bis(di-tert-butyl(4-dimethylaminophenyl)phosphine)dichloropalladium(II) (76.5 mg, 0.11 mmol) and the reaction mixture was heated and stirred at 90° C. for 2 h. The reaction mixture was allowed to cool to RT, poured onto water (40 mL) and the aqueous phase extracted with EtOAc (2×30 mL). The combined organic extracts were dried over Na₂SO₄ and concentrated under vacuum. The crude product was purified by column chromatography on silica gel (using a gradient of 0-100% EtOAc in isohexane as eluant) to afford the title compound as colourless solid (485 mg, yield: 68%). LC-MS m/z [M+H]⁺: 303.1, 305.1; purity: 92%. ¹H NMR (400 MHz, DMSO-d₆) δ 7.69 (d, J=1.8 Hz, 1H), 7.21 (d, J=1.6 Hz, 1H), 7.07 (d, J=1.6 Hz, 1H), 6.36 (d, J=1.8 Hz, 1H), 5.43 (s, 2H), 4.91 (dd, J=9.5, 2.7 Hz, 1H), 3.86-3.76 (m, 1H), 3.35 (ddd, J=10.0, 7.7, 5.2 Hz, 1H), 2.36-2.27 (m, 1H), 1.95 (dt, J=13.0, 4.1 Hz, 1H), 1.86-1.81 (m, 1H), 1.59-1.44 (m, 3H).

### Step 2: Synthesis of 2-chloro-N-[3-chloro-5-cyano-2-(2-tetrahydropyran-2-ylpyrazol-3-yl)phenyl]propenamide #388_2

To a solution of 3-amino-5-chloro-4-(2-tetrahydropyran-2-ylpyrazol-3-yl)benzonitrile #388_1 (150 mg, 0.451 mmol) in DMF (3.0 mL) at 0° C. was added NaH (60.0%, 25 mg, 0.63 mmol) and this mixture was allowed to stir at 0° C. for a further 30 minutes before the dropwise addition of 2-chloropropanoyl chloride (0.061 mL, 0.63 mmol). Upon completion of addition, the reaction mixture was allowed to attain RT and stirred for a further 30 minutes before being carefully quenched by the addition of water (10 mL). The crude product was collected by filtration, washed with water (5 mL) and dried to afford the title compound as a colourless solid (121 mg, yield: 62%). LC-MS m/z [M−H]⁺: 391.1, 393.0; purity: 91%.

### Step 3: Synthesis of 10-chloro-5-(1-chloroethyl)pyrazolo[1,5-c]quinazoline-8-carbonitrile #388_3

To a solution of 2-chloro-N-[3-chloro-5-cyano-2-(2-tetrahydropyran-2-ylpyrazol-3-yl)phenyl]propanamide #388_2 (230 mg, 0.532 mmol) in DCM (8.00 mL) was added TFA (2.0 mL, 27 mmol) and the reaction mixture allowed to stand at RT for 2 h before concentrating under vacuum. The residue was triturated with TBME (3 mL) and the solid collected and dried to give the title compound as an off-white solid (145 mg, yield: 92%). LC-MS m/z [M+H]⁺: 291.1, 293.1; purity: 98%.

### Step 4: Synthesis of 11-chloro-5-methyl-6-oxo-5,7-dihydropyrazolo[1,5-d][1,4]benzodiazepine-9-carbonitrile #388_4

To a suspension of 10-chloro-5-(1-chloroethyl)pyrazolo[1,5-c]quinazoline-8-carbonitrile #388_3 (445 mg, 1.38 mmol) in DMSO (4.0 mL) was added aqueous NaOH (2.0 M, 1.38 mL, 2.75 mmol). The resulting orange solution was stirred at RT for 1 h then water (20 mL) was added followed by AcOH (1.0 mL). The resulting suspension was filtered and the precipitate was washed with water and dried under vacuum. The crude product was purified by column chromatography on silica gel (using a gradient of 0-60% EtOAc in isohexane as eluant) to give the title compound as a colourless solid (350 mg, yield: 91%). LC-MS m/z [M+H]⁺: 273.1, 275.1; purity: 100%. ¹H NMR (400 MHz, DMSO-d₆) δ 10.79 (s, 1H), 8.07 (d, J=1.6 Hz, 1H), 7.67 (d, J=2.0 Hz, 1H), 7.62 (d, J=1.7 Hz, 1H), 6.91 (d, J=2.0 Hz, 1H), 4.88 (q, J=6.6 Hz, 1H), 1.69 (d, J=6.7 Hz, 3H).

### Step 5: Synthesis of 2-(11-chloro-9-cyano-5-methyl-6-oxo-5H-pyrazolo[1,5-d][1,4]benzodiazepin-7-yl)-N-[4-(difluoromethyl)phenyl]acetamide #388

The title product was prepared according to the general procedure 1, starting from 11-chloro-5-methyl-6-oxo-5,7-dihydropyrazolo[1,5-d][1,4]benzodiazepine-9-carbonitrile #388_4 (120 mg, 0.440 mmol) and 2-chloro-N-[4-(difluoromethyl)phenyl]acetamide (CAS 872533-93-2). Following complete conversion, the reaction mixture was quenched by the addition of water (15 mL). The solid was collected by filtration and washed further with water (10 mL) and dried under vacuum. The crude solid was purified by flash reverse phase chromatography on C18 silica gel (using a gradient 5-60% acetonitrile in water+1% HCOOH as eluent) to afford the title product as an off-white solid (160 mg, yield: 77%). LC-MS m/z [M+H]⁺: 456.1, 458.2; purity: 99%. ¹H NMR (400 MHz, DMSO-d₆) δ 10.47 (s, 1H), 8.21 (d, J=1.5 Hz, 1H), 8.11 (d, J=1.6 Hz, 1H), 7.72-7.66 (m, 3H), 7.52 (d, J=8.3 Hz, 2H), 7.12-6.83 (m, 2H), 5.01 (q, J=6.6 Hz, 1H), 4.66-4.48 (m, 2H), 1.69 (d, J=6.7 Hz, 3H).

### Example #389: Enantiomer (7S) or (7R) of 2-[14-chloro-4-fluoro-7,12-dimethyl-8-oxo-5,6,9,13-tetrazatricyclo[8.4.0.0^{2,6}]tetradeca-1(14),2,4,10,12-pentaen-9-yl]-N-[4-(difluoromethyl)phenyl]acetamide

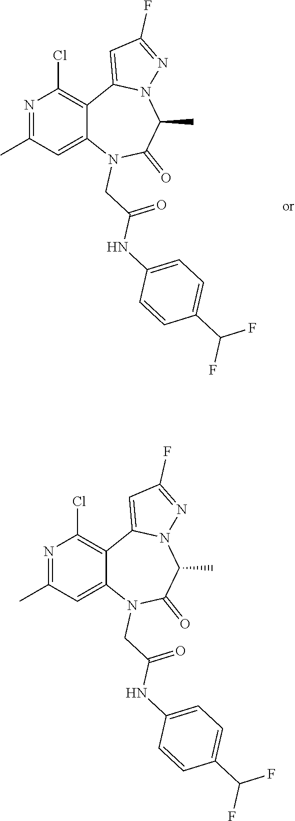 [SMILES: Cc1cc2c(c(Cl)n1)-c1cc(F)nn1[C@@H](C)C(=O)N2CC(=O)Nc1ccc(C(F)F)cc1 ; Cc1cc2c(c(Cl)n1)-c1cc(F)nn1[C@H](C)C(=O)N2CC(=O)Nc1ccc(C(F)F)cc1]

### Step 1: Synthesis of 3-fluoro-1-tetrahydropyran-2-yl-5-(4,4,5,5-tetramethyl-1,3,2-dioxaborolan-2-yl)pyrazole #389_1

To a solution of 3-fluoro-1-tetrahydropyran-2-yl-pyrazole (4.61 g, 26.5 mmol) (CAS 2200261-28-3) in THF (100 mL) at −70° C. was added a solution of n-BuLi in hexanes (2.5 M, 12.7 mL, 31.9 mmol) dropwise. The reaction mixture was stirred at −70° C. for 1 h. Triisopropyl borate (7.4 mL, 31.9 mmol) was added at −78° C. and the reaction mixture was stirred at this temperature for 30 minutes, then allowed to warm to RT and stirred for 1 h. Pinacol (3.76 g, 31.9 mmol) and AcOH (3.1 mL, 54.5 mmol) were added and the reaction mixture was stirred overnight at RT. Water (100 mL) was added and the aqueous phase was extracted with EtOAc (3×75 mL). The combined organics were washed with brine (100 mL), dried over Na₂SO₄, filtered, and concentrated under vacuum. The crude was purified by column chromatography on silica gel (using a gradient of 0-100% TBME in isohexane as eluant) to give the title compound as an off-white solid (7.3 g, yield: 84%). LC-MS m/z [M-166+H]⁺: 131.2; purity: 98% (215 nm). ¹H NMR (400 MHz, CDCl₃) δ 6.22 (d, J=6.0 Hz, 1H), 5.70 (dt, J=10.0, 2.6 Hz, 1H), 4.08-4.01 (m, 1H), 3.64 (td, J=11.3, 2.8 Hz, 1H), 2.40-2.25 (m, 1H), 2.08 (d, J=14.5 Hz, 1H), 1.91 (dd, J=13.5, 3.5 Hz, 1H), 1.68 (qt, J=11.8, 7.9 Hz, 2H), 1.62-1.49 (m, 1H), 1.34 (s, 12H).

### Step 2: Synthesis of 2-chloro-3-(5-fluoro-2-tetrahydropyran-2-yl-pyrazol-3-yl)-6-methyl-pyridin-4-amine #389_2

Nitrogen was sparged through a solution of 3-fluoro-1-tetrahydropyran-2-yl-5-(4,4,5,5-tetramethyl-1,3,2-dioxaborolan-2-yl)pyrazole #389_1 (1.0 g, 3.1 mmol), 3-bromo-2-chloro-6-methyl-pyridin-4-amine N30_1 (1.0 g, 4.7 mmol), CsF (951 mg, 6.26 mmol) and bis(di-tert-butyl(4-dimethylaminophenyl)phosphine)dichloropalladium(II) (332 mg, 0.470 mmol) in 1,4-dioxane (35 mL) and water (7 mL). The reaction mixture was heated at reflux overnight then cooled to RT. The solution was filtered over a pad of celite and washed through with EtOAc (150 mL). The filtrate was concentrated under vacuum. The crude was purified by column chromatography on silica gel (using a gradient of 0-100% EtOAc in isohexane as eluant) to afford the title compound as a yellow wax (950 mg, yield: 59%). LC-MS m/z [M+H]⁺: 311.2, 313.2; rt: 0.79 and 0.92 min (as a mixture of diastereoisomers); purity: 96%.

### Step 3: Synthesis of 2-chloro-N-[2-chloro-3-(5-fluoro-2-tetrahydropyran-2-yl-pyrazol-3-yl)-6-methyl-4-pyridyl]propenamide #389_3

To a solution of 2-chloro-3-(5-fluoro-2-tetrahydropyran-2-yl-pyrazol-3-yl)-6-methyl-pyridin-4-amine #389_2 (1.05 g, 2.0 mmol) in DMF (18 mL) at 0° C. was added NaH (60% purity, 97.3 mg, 2.4 mmol). The solution was stirred for 30 minutes then 2-chloropropanoyl chloride (0.28 mL, 2.84 mmol) was added dropwise. The reaction mixture was stirred for 1 h at 0° C., then allowed to RT and stirred for 1 h. Water (50 mL) was added and the aqueous phase was extracted with EtOAc (3×50 mL). The combined organic extracts were washed with brine (100 mL), dried over Na₂SO₄, filtered and concentrated under reduced pressure. The crude was purified by column chromatography on silica gel (using a gradient of 0-100% EtOAc in isohexane as eluant) to afford the title compound as a yellow oil (183 mg, yield: 21%). LC-MS m/z [M+H]⁺: 401.0, 403.0; rt: 1.16 and 2.00 min (as a mixture of diastereoisomers); purity: 94%.

### Step 4: Synthesis of 13-chloro-7-(1-chloroethyl)-4-fluoro-11-methyl-5,6,8,12-tetrazatricyclo[7.4.0.0^{2,6}]trideca-1(13),2,4,7,9,11-hexaene #389_4

A solution of 2-chloro-N-[2-chloro-3-(5-fluoro-2-tetrahydropyran-2-yl-pyrazol-3-yl)-6-methyl-4-pyridyl]propanamide #389_3 (288 mg, 0.703 mmol) and HCl (4 M in 1,4-dioxane, 2.0 mL, 8.0 mmol) was stirred at RT overnight. The reaction mixture was evaporated to dryness to afford 13-chloro-7-(1-chloroethyl)-4-fluoro-11-methyl-5,6,8,12-tetrazatricyclo[7.4.0.0^{2,6}]trideca-1(13),2,4,7,9,11-hexaene as an off-white solid (155 mg, yield: 64%). LC-MS m/z [M+H]⁺: 299.1, 301.1; purity: 96%. ¹H NMR (400 MHz, DMSO-d₆) δ 7.82 (s, 1H), 7.48 (d, J=5.2 Hz, 1H), 5.93 (q, J=6.7 Hz, 1H), 2.64 (s, 3H), 1.97 (d, J=6.7 Hz, 3H).

### Step 5: Synthesis of 14-chloro-4-fluoro-7,12-dimethyl-5,6,9,13-tetrazatricyclo[8.4.0.0^{2,6}]tetradeca-1(14),2,4,10,12-pentaen-8-one #389_5

To a suspension of 13-chloro-7-(1-chloroethyl)-4-fluoro-11-methyl-5,6,8,12-tetrazatricyclo[7.4.0.0^{2,6}]trideca-1(13),2,4,7,9,11-hexaene #389_4 (155 mg, 0.508 mmol) in DMSO (2.00 mL) was added aqueous NaOH (2 M, 0.51 mL, 1.0 mmol). The resulting orange solution was stirred at RT for 1 h, then water (20 mL) was added followed by AcOH (0.5 mL). The resulting suspension was filtered and the solid was washed with water and dried under vacuum to afford the title compound as an off-white solid (120 mg, yield: 83%). LC-MS m/z [M+H]⁺: 281.1, 283.1; purity: 100%. ¹H NMR (400 MHz, DMSO-d₆) δ 11.06 (s, 1H), 7.06 (s, 1H), 6.58 (d, J=5.8 Hz, 1H), 4.87 (d, J=8.1 Hz, 1H), 2.54 (s, 3H), 1.58 (s, 3H).

### Step 6: Synthesis of 2-(14-chloro-4-fluoro-7,12-dimethyl-8-oxo-5,6,9,13-tetrazatricyclo[8.4.0.0²⁶]tetradeca-1(14),2,4,10,12-pentaen-9-yl)-N-[4-(difluoromethyl)phenyl]acetamide #389

The title product was prepared according to the general procedure 1, starting from 14-chloro-4-fluoro-7,12-dimethyl-5,6,9,13-tetrazatricyclo[8.4.0.0²⁶]tetradeca-1(10),2,4,11,13-pentaen-8-one (60.0 mg, 0.209 mmol) and 2-chloro-N-[4-(difluoromethyl)phenyl]acetamide (CAS 872533-93-2). Following purification by column chromatography on silica gel (using a gradient of 0-80% EtOAc in isohexane) the racemate was separated by chiral SFC (column CHIRALPAK IG from Daicel, eluant 55% MeOH, 45% CO₂) affording the pure enantiomer as an off-white solid (14.2 mg, yield: 56%). LC-MS m/z [M−H]⁻: 462.1, 464.1; purity: 99%. ¹H NMR (400 MHz, DMSO-d₆) δ 10.49 (s, 1H), 7.68 (d, J=8.3 Hz, 2H), 7.55-7.47 (m, 3H), 6.97 (t, J=56.1 Hz, 1H), 6.64 (d, J=5.8 Hz, 1H), 5.00 (qd, J=6.6, 2.2 Hz, 1H), 4.67-4.55 (m, 2H), 2.53 (s, 3H), 1.60 (d, J=6.7 Hz, 3H). Chiral purity: 100%; rt=4.27 min (second eluting enantiomer). For information, first eluting enantiomer rt=1.01 min. Both measured by SFC, CHIRALPAK IG from Daicel, 55% MeOH+0.1% NH₃, 45% CO₂.

### Example #390: 2-(1-methoxy-3-methyl-6-oxo-7H-pyrido[4,3-d][3]benzazepin-5-yl)-N-[4-(trifluoromethyl)phenyl]acetamide

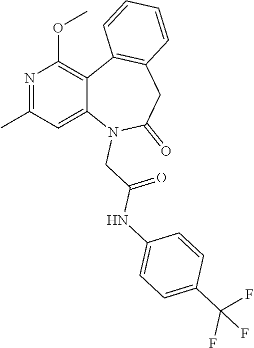 [SMILES: COc1nc(C)cc2c1-c1ccccc1CC(=O)N2CC(=O)Nc1ccc(C(F)(F)F)cc1]

The title product was prepared according to the general procedure 1, starting from 1-methoxy-3-methyl-5,7-dihydropyrido[4,3-d][3]benzazepin-6-one NN350 (20 mg, 0.079 mmol) and 2-chloro-N-[4-(trifluoromethyl)phenyl]acetamide (CAS 2707-23-5). The crude was purified by column chromatography on silica gel (using a gradient of 0-5% MeOH in DCM as eluent) to afford the title compound as a white solid (25 mg, yield: 65%). LC-MS m/z [M+H]⁺: 456.2; purity: 96%. ¹H NMR (400 MHz, DMSO-d₆) δ 10.56 (s, 1H), 7.80 (d, J=8.5 Hz, 2H), 7.74 (q, J=4.0, 3.2 Hz, 1H), 7.69 (d, J=8.6 Hz, 2H), 7.42-7.34 (m, 3H), 6.98 (s, 1H), 4.51 (d, J=16.8 Hz, 1H), 4.42 (d, J=16.8 Hz, 1H), 3.89 (s, 3H), 3.53 (d, J=12.5 Hz, 1H), 3.42 (d, J=12.4 Hz, 1H), 2.45 (s, 3H).

### Example #391: 2-(3,14-difluoro-5-methyl-9-oxo-4,8,12-triazatricyclo[9.4.0.0^{2,7}]pentadeca-1(15),2(7),3,5,11,13-hexaen-8-yl)-N-[4-(trifluoromethyl)phenyl]acetamide

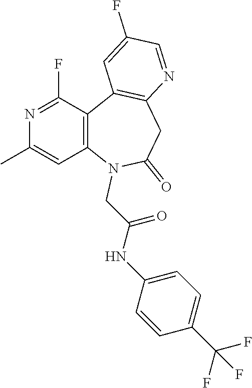 [SMILES: Cc1cc2c(c(F)n1)-c1cc(F)cnc1CC(=O)N2CC(=O)Nc1ccc(C(F)(F)F)cc1]

A solution of 2-fluoro-6-methyl-3-(4,4,5,5-tetramethyl-1,3,2-dioxaborolan-2-yl)pyridin-4-amine N16_1 (30 mg, 0.12 mmol), methyl 2-(3-bromo-5-fluoro-2-pyridyl)acetate (30 mg, 0.12 mmol) and K₃PO₄ (52 mg, 0.24 mmol) in dry Toluene (0.6 mL) was degassed with N2. Pd₂dba₃ (11 mg, 0.012 mmol) and SPhos (5 mg, 0.012 mmol) were added. The reaction mixture was stirred at 100° C. for 18 h. The reaction mixture was diluted with EtOAc, filtered over a pad of celite and concentrated under vacuum. The residue was dissolved in DMF (1 mL). 2-chloro-N-[4-(trifluoromethyl)phenyl]acetamide (21 mg, 0.09 mmol), K2CO₃ (20 mg, 0.14 mmol) and KI (1.5 mg, 0.01 mmol) were added. The reaction mixture was stirred at room temperature for 4 h. Water (3 mL) was added and the reaction mixture was extracted with EtOAc (10 mL). The organic layer was washed with water (4×5 mL), dried over Na₂SO₄, filtered and concentrated under vacuum. The residue was purified by flash chromatography on silica gel (using a gradient of 0 to 100% EtOAc in Heptane as eluent) to afford the title compound (7.4 mg, yield: 13%) as a purple solid. LC-MS m/z: [M+H]⁺: 463.4; purity: 94%. LC-MS (Method B2) m/z: [M+H]⁺: 463.4; rt: 4.43 min; purity: 94%. ¹H NMR (400 MHz, DMSO-d₆) δ 10.61 (s, 1H), 8.66 (d, J=2.8 Hz, 1H), 8.16 (ddd, J=9.7, 4.2, 2.8 Hz, 1H), 7.77 (d, J=8.7 Hz, 2H), 7.68 (d, J=8.7 Hz, 2H), 7.39 (s, 1H), 4.60 (s, 2H), 3.91 (d, J=12.7 Hz, 1H), 3.76 (d, J=12.7 Hz, 1H), 2.54 (s, 3H). ¹⁹F NMR (376 MHz, DMSO-d₆) δ−60.31, −70.32 (d, J=4.2 Hz), −129.68 (d, J=9.7 Hz).

### Example #392: 2-(15-cyano-13-methyl-9-oxo-3,10,14-triazatricyclo[9.4.0.0^{2,7}]pentadeca-1(11),2,4,6,12,14-hexaen-10-yl)-N-[4-(trifluoromethyl)phenyl]acetamide

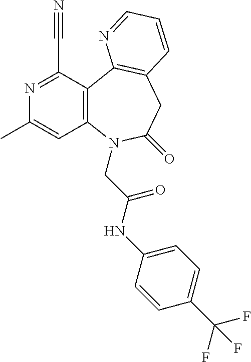 [SMILES: Cc1cc2c(c(C#N)n1)-c1ncccc1CC(=O)N2CC(=O)Nc1ccc(C(F)(F)F)cc1]

### Step 1: Synthesis of ethyl 2-[2-(4-amino-2-cyano-6-methyl-3-pyridyl)-3-pyridyl]acetate NN137

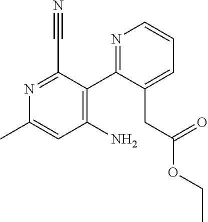 [SMILES: CCOC(=O)Cc1cccnc1-c1c(N)cc(C)nc1C#N]

A solution of 4-amino-3-bromo-6-methyl-pyridine-2-carbonitrile N52 (100 mg, 0.47 mmol), B₂pin₂ (240 mg, 0.95 mmol) and KOAc (140 mg, 1.41 mmol) in dry 1,4-Dioxane (2.3 mL) was degassed with N₂. PCy3 Pd G2 (29 mg, 0.05 mmol) was added at room temperature. The reaction mixture was stirred at 100° C. for 3 h. The reaction mixture was cooled down to room temperature before addition of ethyl 2-(2-bromo-3-pyridyl)acetate (140 mg, 0.57 mmol), K2CO₃ (135 mg, 0.97 mmol) and water (240 μL). The reaction mixture was degassed with N₂ and Pd[(amphos)CI]2 (17 mg, 0.02 mmol) was added. The reaction mixture was then stirred at 100° C. for 16 h. The reaction mixture was quenched by addition of water (10 mL) and extracted with EtOAc (3×15 mL). The combined organic extracts were washed with brine (20 mL), dried over Na₂SO₄, filtered, and concentrated under vacuum. The residue was purified by flash chromatography on silica gel (using a gradient of 0 to 100% EtOAc in Heptane as eluent) to afford the title compound (22 mg, yield: 12%) as an orange solid. LC-MS m/z: [M+H]⁺: 297.0; purity: 77%. ¹H NMR (400 MHz, DMSO-d₆) δ 8.64 (dd, J=4.8, 1.7 Hz, 1H), 7.89 (dd, J=7.8, 1.7 Hz, 1H), 7.49 (dd, J=7.8, 4.8 Hz, 1H), 6.71 (s, 1H), 6.03 (s, 2H), 3.96-3.87 (m, 2H), 3.62 (d, J=16.1 Hz, 1H), 3.49 (d, J=16.1 Hz, 1H), 2.33 (s, 3H), 1.04 (t, J=7.1 Hz, 3H).

### Step 2: Synthesis of 2-(15-cyano-13-methyl-9-oxo-3,10,14-triazatricyclo[9.4.0.0^{2,7}]pentadeca-1(11),2,4,6,12,14-hexaen-10-yl)-N-[4-(trifluoromethyl)phenyl]acetamid #392

To a solution of ethyl 2-[2-(4-amino-2-cyano-6-methyl-3-pyridyl)-3-pyridyl]acetate NN137 (20 mg, 0.05 mmol) in dry toluene (0.3 mL) was added at room temperature LiHMDS (1.5 M in THF, 100 μL, 0.15 mmol). The solution was stirred at room temperature for 1 h. The reaction mixture was quenched by addition of saturated aqueous NH₄Cl solution (5 mL) and extracted with EtOAc (3×10 mL). The combined organic extracts were washed with brine (10 mL), dried over Na₂SO₄, filtered, and concentrated under vacuum. The residue was dissolved in DMF (0.8 mL). 2-chloro-N-[4-(trifluoromethyl)phenyl]acetamide (15 mg, 0.06 mmol), K2CO₃ (14 mg, 0.10 mmol) and KI (2 mg, 0.01 mmol) were added. The reaction mixture was stirred at room temperature for 5 h. Water (3 mL) was added and the reaction mixture was extracted with EtOAc (10 mL). The organic layer was washed with water (3×5 mL), dried over Na₂SO₄, filtered and concentrated under vacuum. The residue was purified by preparative HPLC Purification Method P_A) then by SFC (Kromasil SFC-5-Diol, CO₂+MeOH) to afford the title compound (1 mg, yield: 4%) as a white solid. LC-MS m/z: [M+H]⁺: 452; purity: 99%.

### Example #393: 2-(1-chloro-9-methoxy-3,7-dimethyl-6-oxo-7H-pyrido[3,4-a][3]benzazepin-5-yl)-N-[4-(trifluoromethyl)phenyl]acetamide

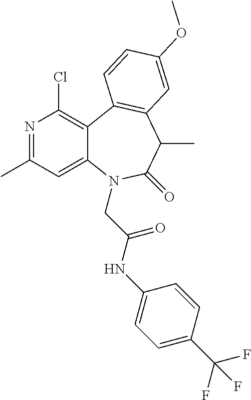 [SMILES: COc1ccc2c(c1)C(C)C(=O)N(CC(=O)Nc1ccc(C(F)(F)F)cc1)c1cc(C)nc(Cl)c1-2]

### Step 1: Synthesis of ethyl 2-(2-bromo-5-methoxy-phenyl)propanoate #393_1

To a solution of ethyl 2-(2-bromo-5-methoxy-phenyl)acetate (324 mg, 1.19 mmol) (CAS 30414-82-5) in THF (3 mL) at 0° C. was added LiHMDS in THF (1 M, 1.30 mL, 1.30 mmol) dropwise over 10 minutes, and the reaction mixture was stirred at 0° C. for 45 min. Mel (219 mg, 1.54 mmol) was added dropwise, then the reaction mixture stirred at 0° C. for 1 h and then at RT for a further 1 h. Saturated aqueous NH₄Cl (10 mL) was added and the aqueous phase was extracted with EtOAc (3×10 mL). The organic layers were combined, washed with brine (10 mL) then dried over Na₂SO₄. The volatiles were removed under vacuum and the residue purified by column chromatography on silica gel (using a gradient of 0-10% EtOAc in isohexane as eluant) to give the title compound as a colourless oil (264 mg, yield: 74%). ¹H NMR (400 MHz, DMSO-d₆) δ 7.51 (d, J=8.6 Hz, 1H), 7.01-6.69 (m, 2H), 4.07 (dq, J=14.4, 7.1 Hz, 3H), 3.74 (s, 3H), 1.39 (d, J=7.2 Hz, 3H), 1.14 (t, J=7.1 Hz, 3H).

### Step 2: Synthesis of ethyl 2-[5-methoxy-2-(4,4,5,5-tetramethyl-1,3,2-dioxaborolan-2-yl)phenyl]propanoate #393_2

To a suspension of ethyl 2-(2-bromo-5-methoxy-phenyl)propanoate #393_1 (110 mg, 0.383 mmol), bis(pinacolato)diboron (117 mg, 0.460 mmol), CH₃COOK (150 mg, 1.53 mmol), 3 Å molecular sieves (200 mg) in 1,4-dioxane (3 mL) under a nitrogen atmosphere was added Pd(dppf)Cl₂ (14.0 mg, 0.0192 mmol). The suspension was heated at reflux overnight, then it was filtered over a pad of celite and washed with through with EtOAc (5 mL). The filtrate was concentrated to give the title compound as a brown oil (230 mg, yield: 99%). LC-MS m/z [M+H]⁺: 335.2; purity: 70%.

### Step 3: Synthesis of 2-(1-chloro-9-methoxy-3,7-dimethyl-6-oxo-7H-pyrido[3,4-a][3]benzazepin-5-yl)-N-[4-(trifluoromethyl)phenyl]acetamide #393

The title product was prepared according to the general procedure 1, starting from 1-chloro-9-methoxy-3,7-dimethyl-5,7-dihydropyrido[3,4-a][3]benzazepin-6-one NN351 prepared starting from #393_2 and N30_1 according to already described procedures (43.0 mg, 0.121 mmol) and 2-chloro-N-[4-(trifluoromethyl)phenyl]acetamide (CAS 2707-23-5). The crude product was purified by column chromatography on silica gel (using a gradient of 0-100% EtOAc in isohexane as eluant) to obtain the title compound as an off-white solid (4.4 mg, yield: 7%). LC-MS m/z [M+H]⁺: 504.2, 506.2; purity: 95%. ¹H NMR (400 MHz, DMSO-d₆) δ 10.57 (s, 1H), 7.78 (d, J=8.6 Hz, 2H), 7.69 (dd, J=8.7, 5.2 Hz, 3H), 7.36 (s, 1H), 7.00 (dd, J=8.7, 2.6 Hz, 1H), 6.84 (d, J=2.6 Hz, 1H), 4.51 (s, 2H), 3.84 (s, 3H), 3.52 (q, J=6.6 Hz, 1H), 2.50 (s, 3H), 1.43 (d, J=6.7 Hz, 3H).

### Example #394: Enantiomer (5S) or (5R) of N-(4-chloro-3-fluoro-phenyl)-2-[9-cyano-10-fluoro-5-methyl-6-oxo-5H-pyrido[2,3-d][1]benzazepin-7-yl]acetamide

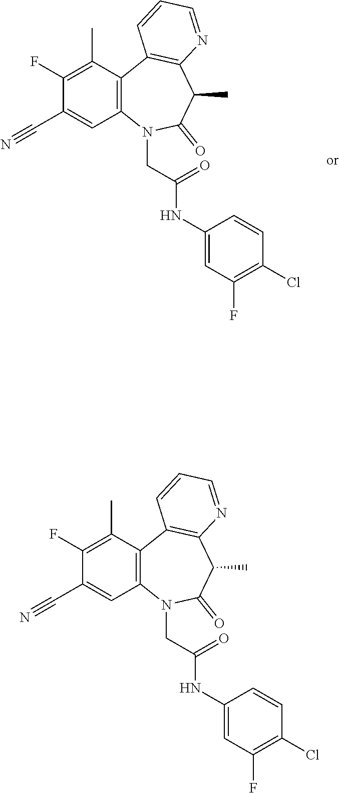 [SMILES: Cc1c(F)c(C#N)cc2c1-c1cccnc1[C@@H](C)C(=O)N2CC(=O)Nc1ccc(Cl)c(F)c1 ; Cc1c(F)c(C#N)cc2c1-c1cccnc1[C@H](C)C(=O)N2CC(=O)Nc1ccc(Cl)c(F)c1]

The title compound was prepared according to the general procedure 1 starting from 10-fluoro-5-methyl-6-oxo-5,7-dihydropyrido[2,3-d][1]benzazepine-9-carbonitrile NN234 and 2-chloro-N-(4-chloro-3-fluoro-phenyl)acetamide (118 mg, 0.531 mmol). The residue was taken in EtOAc, sonicated and the precipitate was filtered and dried under vacuum to give the title compound (146 mg, yield: 73%) as a white solid. The racemate (127 mg) was separated by chiral chromatography (SFC Chiralpak AD, CO₂+Ethanol 25%) to yield the pure enantiomer (48.5 mg, yield: 38.2%) as a white solid. Chiral purity: 99.2%; rt=2.82 min (second eluting enantiomer). For information, first eluting enantiomer rt=1.82. Both measured by HPLC, Chiralpack IA, solvent: EtOH 50%—Heptane 50%—DEA 0.1%). LC-MS m/z [M+H]⁺: 453.2/455.2; purity 100%. ¹H NMR (400 MHz, DMSO-d₆) δ 10.56 (s, 1H), 8.72 (d, J=4.8 Hz, 1H), 8.19 (d, J=7.9 Hz, 1H), 8.15 (d, J=5.9 Hz, 1H), 7.93 (d, J=10.1 Hz, 1H), 7.72 (d, J=11.8 Hz, 1H), 7.59-7.54 (m, 1H), 7.51 (d, J=8.5 Hz, 1H), 7.30 (d, J=8.8 Hz, 1H), 4.66-4.43 (m, 2H), 3.70 (q, J=6.6 Hz, 1H), 1.48 (d, J=6.6 Hz, 3H).

### Example #395: 2-(3,5-dichloro-10-methyl-9-oxo-4,8,12-triazatricyclo[9.4.0.0^{2,7}]pentadeca-1(11),2(7),3,5,12,14-hexaen-8-yl)-N-[4-(difluoromethyl)phenyl]acetamide

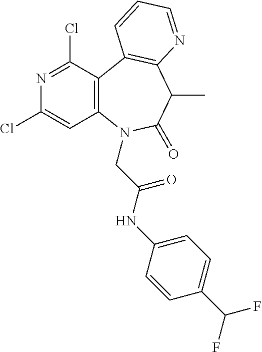 [SMILES: CC1C(=O)N(CC(=O)Nc2ccc(C(F)F)cc2)c2cc(Cl)nc(Cl)c2-c2cccnc21]

The title compound was prepared according to the general procedure 1 starting from 3,5-dichloro-10-methyl-4,8,12-triazatricyclo[9.4.0.0^{2,7}]pentadeca-1(11),2(7),3,5,12,14-hexaen-9-one NN167 (63.0 mg, 0.167 mmol) and 2-chloro-N-[4-(difluoromethyl)phenyl]acetamide (44.0 mg, 0.201 mmol). The residue was purified by preparative HPLC (Purification Method P_A). The residue was solubilized in DCM (50 mL) and washed with a saturated aqueous solution of Na₂CO₃ (2×20 mL). The organic layer was dried with MgSO₄, filtered and concentrated to dryness to afford the title compound as a white solid (45.3 mg, yield: 55%). LC-MS (Method A2) m/z [M+H]+: 477.1/479.0/481.1; rt: 4.65 min; purity: 97%. LC-MS m/z [M+H]⁺: 477.1/479.1/481.1; purity: 96%. ¹H NMR (400 MHz, DMSO-d₆) δ 10.49 (s, 1H), 8.71-8.66 (m, 1H), 8.27 (d, J=7.9 Hz, 1H), 7.75 (s, 1H), 7.70-7.64 (m, 2H), 7.55-7.47 (m, 3H), 6.97 (t, J=56.1 Hz, 1H), 4.72-4.50 (m, 2H), 3.94 (q, J=6.6 Hz, 1H), 1.48 (d, J=6.6 Hz, 3H). ¹⁹F NMR (376 MHz, DMSO-d₆) δ−108.11 (d, J=56.1 Hz).

### Example #396: Enantiomer (7S) or (7R) of 2-[9-chloro-3,7-dimethyl-6-oxo-7H-pyrido[3,4-a][3]benzazenin-5-yl]-N-[4-(difluoromethyl)phenyl]acetamide

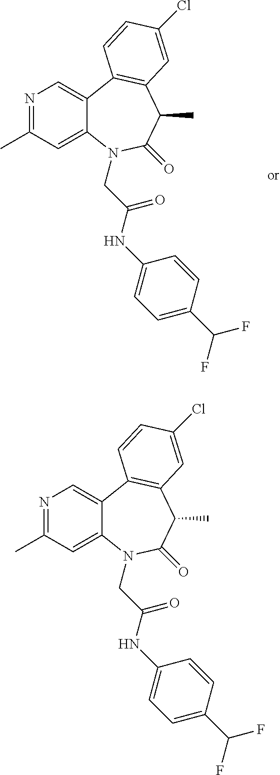

The title product was prepared according to the general procedure 1, starting from 9-chloro-3,7-dimethyl-5,7-dihydropyrido[4,3-d][3]benzazepin-6-one NN352 (66 mg, 0.23 mmol) and 2-chloro-N-[4-(difluoromethyl)phenyl]acetamide (CAS 872533-93-2). The residue was purified by column chromatography on silica gel (using a gradient of 0-100% EtOAc in isohexane as eluent). The racemate was the separated by Chiral SFC (column CHIRALPAK IH from Daicel, eluant 40% MeOH+0.1% NH₃, 60% CO₂) to yield the pure enantiomer as a white solid (24 mg, yield: 30%). LC-MS m/z [M+H]⁺: 456.2; purity: 95%. ¹H NMR (400 MHz, DMSO-d₆) δ 10.45 (s, 1H), 8.68 (s, 1H), 7.73 (d, J=8.3 Hz, 1H), 7.70 (d, J=8.4 Hz, 2H), 7.55 (dd, J=8.3, 2.2 Hz, 1H), 7.51 (d, J=8.4 Hz, 2H), 7.40 (d, J=2.2 Hz, 1H), 7.34 (s, 1H), 6.97 (t, J=56.2 Hz, 1H), 4.61 (d, J=16.8 Hz, 1H), 4.51 (d, J=16.7 Hz, 1H), 3.48 (q, J=6.6 Hz, 1H), 2.54 (s, 3H), 1.46 (d, J=6.8 Hz, 3H). Chiral purity: 98%; rt=1.84 min (First eluting enantiomer). For information, second eluting enantiomer rt=2.53 min. Both measured by HPLC, CHIRALPAK IH from Daicel, 25% MeOH+0.1% NH₃, 75% CO₂.

### Example #397: Enantiomer (7S) or (7R) of N-(4-chloro-3-fluoro-phenyl)-2-[1-hydroxy-7-methyl-6-oxo-3-(trifluoromethyl)-7H-pyrido[3,4-a][3]benzazepin-5-yl]acetamide

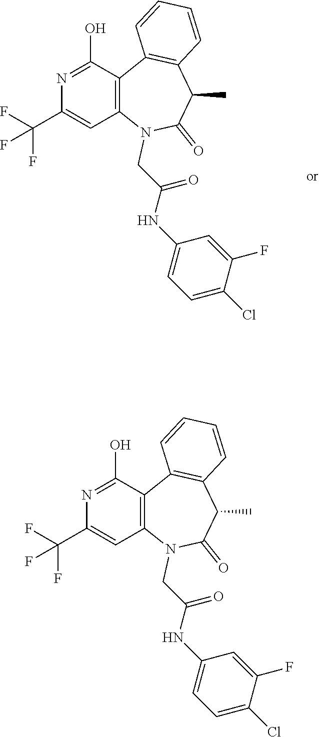 [SMILES: C[C@H]1C(=O)N(CC(=O)Nc2ccc(Cl)c(F)c2)c2cc(C(F)(F)F)nc(O)c2-c2ccccc21 ; C[C@@H]1C(=O)N(CC(=O)Nc2ccc(Cl)c(F)c2)c2cc(C(F)(F)F)nc(O)c2-c2ccccc21]

### Step 1: Synthesis of 3-bromo-2-chloro-6-(trifluoromethyl)pyridin-4-amine #397_1

2-chloro-6-(trifluoromethyl)pyridin-4-amine (CAS 34486-22-1) (100 mg, 0.51 mmol) was dissolved in DCM (5.0 mL). NBS (81.5 mg, 0.46 mmol) was dissolved in DCM (5.0 mL) and added dropwise to the reaction mixture which was then stirred overnight at room temperature. The reaction mixture was concentrated under vacuum and the crude was purified by column chromatography on silica gel (using a gradient of 0-100% EtOAc in heptane as eluent) to afford the title compound as a white flaky solid (109 mg, yield: 75%). LC-MS m/z [M+H]⁺: 275.0, 277.0; purity: 98%. ¹H NMR (400 MHz, DMSO-d₆) δ 7.19 (s, 2H), 7.06 (s, 1H).

### Step 2: Synthesis of 3-bromo-2-methoxy-6-(trifluoromethyl)pyridin-4-amine #397_2

To a solution of 3-bromo-2-chloro-6-(trifluoromethyl)pyridin-4-amine #397_1 (1.18 g, 3.93 mmol) in NMP (20.0 mL) was added NaOMe in MeOH (5.40 mol/L, 1.1 mL, 5.9 mmol) dropwise. The resulting mixture was stirred at 100° C. for 6 h. NaOMe in MeOH (5.40 mol/L, 1.1 mL, 5.9 mmol) was added and the reaction mixture was stirred at 100° C. for 6 h. The reaction was then cooled to RT and treated with EtOAc (50 mL). The organic phase was separated and washed with LiCl 1M (2×20 mL) then with brine (2×20 mL). The organic phase was dried over Na₂SO₄, filtered and concentrated under reduced pressure. The crude was purified by column chromatography on silica gel (using a gradient of 0-100% EtOAc in heptane as eluent) to afford the title compound as a pink solid

### Step 3: Synthesis of N-(4-chloro-3-fluoro-phenyl)-2-[1-methoxy-7-methyl-6-oxo-3-(trifluoromethyl)-7H-pyrido[4,3-d][3]benzazepin-5-yl]acetamide #397_3

A solution of 1-methoxy-7-methyl-3-(trifluoromethyl)-5,7-dihydropyrido[4,3-d][3]benzazepin-6-one NN353 prepared from #397_2 and N64_2 according to previously described procedures (150 mg, 0.45 mmol), K2CO₃ (124 mg, 0.89 mmol), KI (15 mg, 0.1 mmol) and 2-chloro-N-(4-chloro-3-fluoro-phenyl)acetamide (122 mg, 0.54 mmol) in DMF (4.0 mL) was stirred at room temperature overnight. The reaction was treated with EtOAc (30 mL). The organic phase was separated and washed with LiCl 1M (2×10 mL) then with brine (2×10 mL). The organic phase was dried over Na₂SO₄, filtered and concentrated under reduced pressure. The crude was purified by column chromatography on silica gel (using a gradient of 0-100% EtOAc in isohexane as eluent) to afford the title compound as a light yellow gum (85 mg, yield: 32%). LC-MS m/z [M+H]⁺: 508.2; purity: 87%.

### Step 4: Synthesis of N-(4-chloro-3-fluoro-phenyl)-2-[1-hydroxy-7-methyl-6-oxo-3-(trifluoromethyl)-7H-pyrido[4,3-d][3]benzazepin-5-yl]acetamide #397

N-(4-chloro-3-fluoro-phenyl)-2-[1-methoxy-7-methyl-6-oxo-3-(trifluoromethyl)-7H-pyrido[4,3-d][3]benzazepin-5-yl]acetamide #397_3 (85 mg, 0.15 mmol) and KI (120 mg, 0.72 mmol) were suspended in dry MeCN (5 mL) and TMSCl (0.091 mL, 0.72 mmol) was then added. The reaction mixture was stirred at 80° C. overnight. The reaction was let to cool to room temperature then treated with a 1/1 mixture of brine and a saturated NaHCO₃ solution (20 mL) and extracted with DCM-MeOH 9:1 (5×20 mL). The organic phase was dried over Na₂SO₄, filtered and concentrated under reduced pressure. After purification by column chromatography on silica gel (using a gradient of 0-100% EtOAc in isohexane as eluent), the racemate was separated by Chiral SFC (column CHIRALPAK IG from Daicel, eluant 25% MeOH+0.03% NH₃, 75% CO₂) to yield the pure enantiomer as a white solid (10.9 mg, yield: 21%). LC-MS m/z [M+H]⁺: 494.1, 496.0; purity: 100%. ¹H NMR (400 MHz, DMSO-d₆) δ 12.35 (s, 1H), 10.58 (s, 1H), 7.86 (d, J=7.7 Hz, 1H), 7.73 (dd, J=11.8, 2.4 Hz, 1H), 7.63-7.19 (m, 6H), 4.64-4.31 (m, 2H), 3.59-3.40 (m, 1H), 1.45 (d, J=6.8 Hz, 3H). Chiral purity: 99%; rt=2.70 min (second eluting enantiomer). For information, first eluting enantiomer rt=1.62 min. Both measured by SFC, (CHIRALPAK IG from Daicel, 75% CO₂ and 25% MeOH+0.1% NH₃).

### Example #398: Enantiomer (10S) or (10R) of N-[4-(difluoromethyl)phenyl]-2-[3-chloro-10-methyl-9-oxo-4,8,12-triazatricyclo[9.4.0.0^{2,7}]pentadeca-1(11),2(7),3,5,12,14-hexaen-8-yl]acetamide

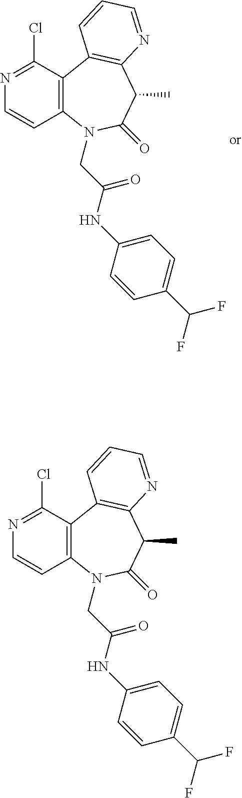 [SMILES: C[C@@H]1C(=O)N(CC(=O)Nc2ccc(C(F)F)cc2)c2ccnc(Cl)c2-c2cccnc21 ; C[C@H]1C(=O)N(CC(=O)Nc2ccc(C(F)F)cc2)c2ccnc(Cl)c2-c2cccnc21]

The title product was prepared according to the general procedure 1, starting from 3-chloro-10-methyl-4,8,12-triazatricyclo[9.4.0.0^{2,7}]pentadeca-1(11),2(7),3,5,12,14-hexaen-9-one NN168 (500.0 mg, 1.906 mmol) and 2-chloro-N-[4-(difluoromethyl)phenyl]acetamide (502.0 mg, 2.286 mmol. The residue was purified by flash chromatography on silica gel (using a gradient of 0 to 100% EtOAc:Heptane then 100% EtOAc as eluent) to afford the title compound as a white solid (784 mg, yield: 87%). A part of the racemate (100 mg) was separated by chiral chromatography (SFC CHIRALPAK AD from Daicel, eluant: CO₂+iPrOH 20%) to afford the title compound as a white solid (41.9 mg, yield: 44%). Chiral purity: 100%; rt=4.23 min (second eluting enantiomer). For information, first eluting enantiomer rt=1.63 min. Both measured by HPLC (CHIRALPAK IA from Daicel, eluant: EtOH 100%—DEA 0.1%). LC-MS m/z [M+H]⁺: 443/445; purity: 99%. ¹H NMR (400 MHz, DMSO-d₆) δ 10.47 (s, 1H), 8.68 (dd, J=4.8, 1.7 Hz, 1H), 8.48 (d, J=5.7 Hz, 1H), 8.27 (dd, J=7.9, 1.7 Hz, 1H), 7.70-7.64 (m, 2H), 7.57 (d, J=5.7 Hz, 1H), 7.54-7.47 (m, 3H), 6.96 (t, J=56.1 Hz, 1H), 4.65-4.52 (m, 2H), 3.83 (q, J=6.6 Hz, 1H), 1.49 (d, J=6.6 Hz, 3H). ¹⁹F NMR (376 MHz, DMSO-d₆) δ−108.10 (d, J=56.1 Hz).

### Example #399: N-(4-chloro-3-fluoro-phenyl)-2-[3-(hydroxymethyl)-2-methoxy-7-methyl-6-oxo-7H-pyrido[3,2-d][3]benzazepin-5-yl]acetamide

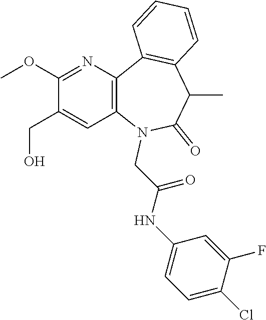 [SMILES: COc1nc2c(cc1CO)N(CC(=O)Nc1ccc(Cl)c(F)c1)C(=O)C(C)c1ccccc1-2]

The title product was prepared according to the general procedure 1, starting from 3-(hydroxymethyl)-2-methoxy-7-methyl-5,7-dihydropyrido[3,2-d][3]benzazepin-6-one NN354 (20 mg, 0.067 mmol) prepared from intermediates N64_2 and NN247 according to already described procedures and 2-chloro-N-(4-chloro-3-fluoro-phenyl)acetamide (CAS 895641-02-8). The residue was purified by column chromatography on silica gel (using a gradient of 0-70% EtOH-EtOAc 1:3 in isohexane as eluant) to afford the title compound as an off-white solid (4.6 mg, yield: 25%). LC-MS m/z [M+H]⁺: 470.1, 472.1; purity: 97%. ¹H NMR (400 MHz, DMSO-d₆) δ 10.54 (s, 1H), 7.99 (dd, J=7.8, 1.6 Hz, 1H), 7.88 (s, 1H), 7.76 (dd, J=11.9, 2.4 Hz, 1H), 7.57-7.46 (m, 3H), 7.39-7.30 (m, 2H), 5.33 (t, J=5.5 Hz, 1H), 4.56-4.43 (m, 3H), 4.25 (d, J=16.7 Hz, 1H), 3.98 (s, 3H), 3.46 (q, J=6.7 Hz, 1H), 1.48 (d, J=6.8 Hz, 3H). ¹⁹F NMR (376 MHz, DMSO-d₆) δ−114.63.

### Example #400: N-(4-chloro-3-fluoro-phenyl)-2-(4-fluoro-1-methoxy-7-methyl-6-oxo-7H-pyrido[4,3-d][3]benzazepin-5-yl)acetamide

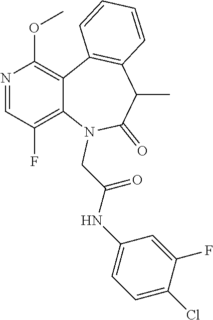 [SMILES: COc1ncc(F)c2c1-c1ccccc1C(C)C(=O)N2CC(=O)Nc1ccc(Cl)c(F)c1]

### Step 1: Synthesis of 3-bromo-5-fluoro-2-methoxy-pyridin-4-amine #400_1

A solution of 5-fluoro-2-methoxy-pyridin-4-amine (CAS 58381-05-8) (867 mg, 6.1 mmol) and NBS (1.3 g, 7.36 mmol) in DCM (30 mL) was stirred at rt for 2 h then washed with water (20 mL) and dried by filtration through a hydrophobic frit. The volatiles were removed under vacuum to afford the title compound as a brown oil (1.3 g, yield: 96%). LC-MS m/z [M+H]⁺: 221.0, 223.2; purity: 100%. ¹H NMR (400 MHz, DMSO-d₆) δ 7.76 (d, J=2.4 Hz, 1H), 6.41 (s, 2H), 3.80 (s, 3H).

### Step 2: Synthesis of N-(4-chloro-3-fluoro-phenyl)-2-(4-fluoro-1-methoxy-7-methyl-6-oxo-7H-pyrido[4,3-d][3]benzazepin-5-yl)acetamide #400

The title product was prepared according to the general procedure 1, starting from 4-fluoro-1-methoxy-7-methyl-5,7-dihydropyrido[4,3-d][3]benzazepin-6-one NN355 (30 mg, 0.11 mmol) prepared starting from intermediates #400_1 and N64_2 according to already described procedures and 2-chloro-N-(4-chloro-3-fluoro-phenyl)acetamide (CAS 895641-02-8). The residue was purified by column chromatography on silica gel (using a gradient of 0-80% EtOAc in isohexane as eluant) to afford the title compound as a colourless solid (27 mg, yield: 54%). LC-MS m/z [M+Na]⁺: 480.2, 482.2; purity: 100%. ¹H NMR (400 MHz, DMSO-d₆) δ 10.28 (s, 1H), 8.30 (d, J=2.8 Hz, 1H), 7.79 (dd, J=7.9, 1.4 Hz, 1H), 7.59-7.31 (m, 5H), 7.18 (ddd, J=8.8, 2.4, 1.0 Hz, 1H), 4.62 (d, J=16.7 Hz, 1H), 4.26 (dd, J=16.6, 2.2 Hz, 1H), 3.88 (s, 3H), 3.58 (q, J=6.7 Hz, 1H), 1.47 (d, J=6.8 Hz, 3H).

### Example #401: Enantiomer (8S) or (8R) of N-(4-chloro-3-fluoro-phenyl)-2-[15-fluoro-8,13-dimethyl-9-oxo-3,10,14-triazatricyclo[9.4.0.0^{2,7}]pentadeca-1(15),2,4,6,11,13-hexaen-10-yl]acetamide

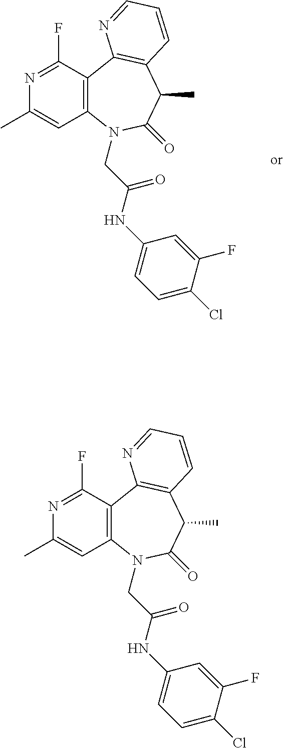 [SMILES: Cc1cc2c(c(F)n1)-c1ncccc1[C@@H](C)C(=O)N2CC(=O)Nc1ccc(Cl)c(F)c1 ; Cc1cc2c(c(F)n1)-c1ncccc1[C@H](C)C(=O)N2CC(=O)Nc1ccc(Cl)c(F)c1]

The title product was prepared according to the general procedure 1, starting from Intermediate NN331 (150 mg, 0.57 mmol) and 2-chloro-N-(4-chloro-3-fluoro-phenyl)acetamide (CAS 895641-02-8). After purification by column chromatography on silica gel, the racemate was separated by chiral SFC (column Lux i-Cellulose-5 from Phenomenex, eluent 30% MeOH+0.03% NH₃, 70% CO₂) to yield the pure enantiomer as off-white solid (39 mg, yield: 16%) LC-MS m/z [M+H]⁺: 443.1; purity: 100%. ¹H NMR (400 MHz, DMSO-d₆) δ 10.57 (s, 1H), 8.67 (dd, J=4.7, 1.5 Hz, 1H), 7.83 (d, 1H), 7.74 (dd, J=11.9, 2.4 Hz, 1H), 7.58-7.46 (m, 2H), 7.37-7.27 (m, 2H), 4.55 (s, 2H), 3.69 (dd, J=6.8, 6.7 Hz, 1H), 1.47 (d, J=6.8 Hz, 3H), three protons obscured by the solvent peak. Chiral purity: 100%; rt=2.43 min (second eluting enantiomer). For information, first eluting enantiomer rt=1.69 min. Both measured by chiral SFC, Lux i-Cellulose-5 from Phenomenex, eluent 30% MeOH+0.03% NH₃, 70% CO₂.

### Example #402: N-(4-chloro-3-fluoro-phenyl)-2-(1-fluoro-3,7-dimethyl-6-oxo-7H-pyrido[3,4-a][3]benzazepin-5-yl)acetamide

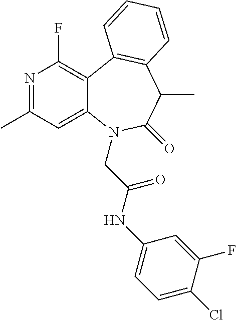 [SMILES: Cc1cc2c(c(F)n1)-c1ccccc1C(C)C(=O)N2CC(=O)Nc1ccc(Cl)c(F)c1]

The title product was prepared according to general procedure 1, starting from 1-fluoro-3,7-dimethyl-5,7-dihydropyrido[4,3-d][3]benzazepin-6-one NN330 and 2-chloro-N-(4-chloro-3-fluoro-phenyl)acetamide (CAS 895641-02-8). Purification by column chromatography on silica gel (using a gradient of 0-100% EtOAc in isohexane as eluent) and reverse phase HPLC (acidic elution) afforded the title compound as a colourless solid (3.0 mg, yield: 4%). LC-MS m/z [M+H]⁺: 442.1; purity: 96%. ¹H NMR (400 MHz, DMSO-d₆) δ 10.58 (s, 1H), 7.80-7.72 (m, 1H), 7.72-7.64 (m, 1H), 7.53 (t, J=8.8 Hz, 2H), 7.48-7.38 (m, 2H), 7.32 (d, J=10.2 Hz, 2H), 4.59-4.44 (m, 2H), 3.57 (t, J=6.8 Hz, 1H), 3.29 (s, 3H), 1.47 (d, J=6.8 Hz, 3H).

### Example #403: Enantiomer (10S) or (10R) of N-(4-chloro-3-fluoro-phenyl)-2-[3,14-difluoro-5-methoxy-10-methyl-9-oxo-4,8,12-triazatricyclo[9.4.0.0^{2,7}]pentadeca-1(11),2(7),3,5,12,14-hexaen-8-yl]acetamide

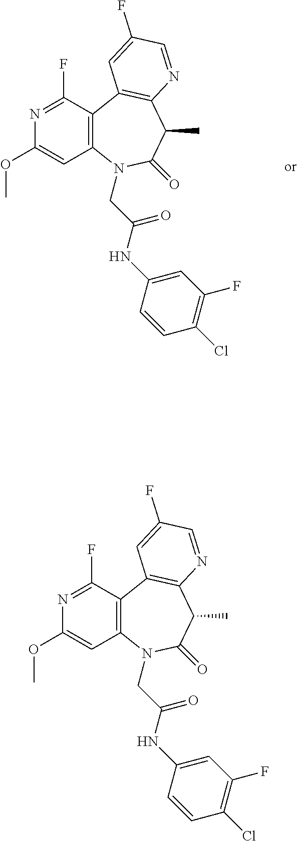 [SMILES: COc1cc2c(c(F)n1)-c1cc(F)cnc1[C@@H](C)C(=O)N2CC(=O)Nc1ccc(Cl)c(F)c1 ; COc1cc2c(c(F)n1)-c1cc(F)cnc1[C@H](C)C(=O)N2CC(=O)Nc1ccc(Cl)c(F)c1]

The title product was prepared according to general procedure 1, starting from 3,14-difluoro-5-methoxy-10-methyl-4,8,12-triazatricyclo[9.4.0.0^{2,7}]pentadeca-1(11),2(7),3,5,12,14-hexaen-9-one N40A (47 mg, 0.16 mmol) and 2-chloro-N-(4-chloro-3-fluoro-phenyl)acetamide (52 mg, 0.24 mmol). The residue was purified by preparative HPLC (Waters XBridge OBD MS C18 column using NH₄HCO₃ 10 mM+50 μl/L NH₄OH in water/MeCN (0 to 100%) as eluent) and then by liquid chiral chromatography (Chiralpak IA from Daicel, using Heptane-EtOH 1:1 as solvents) to give the compound as a white solid (15 mg, Yield: 19.5%) and the other enantiomer as white solid (14 mg, Yield=18%). LC-MS m/z [M+H]⁺: 477; purity: 99%. ¹H NMR (400 MHz, DMSO-d₆) δ 10.59 (s, 1H), 8.67 (d, J=2.8 Hz, 1H), 8.10 (dt, J=9.7, 3.4 Hz, 1H), 7.71 (dd, J=11.8, 2.4 Hz, 1H), 7.52 (t, J=8.6 Hz, 1H), 7.30 (dd, J=9.0, 2.4 Hz, 1H), 6.91 (s, 1H), 4.59 (d, J=2.8 Hz, 2H), 3.93 (s, 3H), 3.87 (q, J=6.6 Hz, 1H), 1.47 (d, J=6.6 Hz, 3H). Chiral purity: 100%; rt=2.18 min (second eluting enantiomer) column HPLC ChiralPak IA with Heptane/EtOH 1:1+0.1% DEA as eluent. For information, first eluting enantiomer rt=1.56 min (100%).

### Example #404: N-(4-chloro-3-fluoro-phenyl)-2-(1,9-difluoro-3,7-dimethyl-6-oxo-7H-pyrido[3,4-a][3]benzazepin-5-yl)acetamide

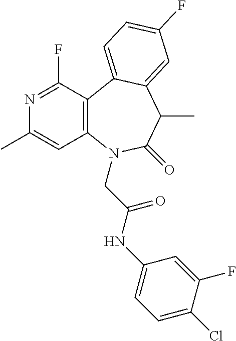 [SMILES: Cc1cc2c(c(F)n1)-c1ccc(F)cc1C(C)C(=O)N2CC(=O)Nc1ccc(Cl)c(F)c1]

The title product was prepared according to general procedure 1, starting from 1,9-difluoro-3,7-dimethyl-5,7-dihydropyrido[4,3-d][3]benzazepin-6-one NN356 and 2-chloro-N-(4-chloro-3-fluoro-phenyl)acetamide (CAS 895641-02-8). Purification by column chromatography on silica gel (using a gradient of 0-100% EtOAc in isohexane as eluent) afforded the title compound as a cream coloured solid (68 mg, yield: 42%). LC-MS m/z [M+H]⁺: 460.1, 462.1; purity: 99.5%. ¹H NMR (400 MHz, DMSO-d₆) δ 10.57 (s, 1H), 7.79-7.68 (m, 2H), 7.52 (t, J=8.7 Hz, 1H), 7.35-7.20 (m, 4H), 4.58-4.47 (m, 2H), 3.59 (q, J=6.6 Hz, 1H), 2.48 (s, 3H), 1.45 (d, J=6.7 Hz, 3H).

### Example #405: Enantiomer (7S) or (7R) of N-(4-chloro-3-fluoro-phenyl)-2-[1,10-difluoro-3,7-dimethyl-6-oxo-7H-pyrido[3,4-a][3]benzazepin-5-yl]acetamide

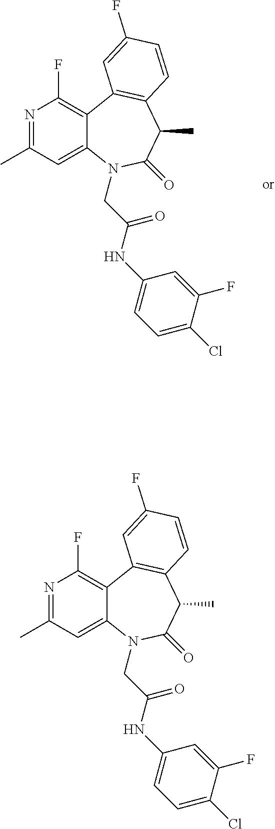 [SMILES: Cc1cc2c(c(F)n1)-c1cc(F)ccc1[C@@H](C)C(=O)N2CC(=O)Nc1ccc(Cl)c(F)c1 ; Cc1cc2c(c(F)n1)-c1cc(F)ccc1[C@H](C)C(=O)N2CC(=O)Nc1ccc(Cl)c(F)c1]

The title product was prepared according to the general procedure 1, starting from 1,10-difluoro-3,7-dimethyl-5,7-dihydropyrido[3,4-a][3]benzazepin-6-one NN357 (200 mg, 0.656 mmol) and 2-chloro-N-(4-chloro-3-fluoro-phenyl)acetamide (CAS 895641-02-8). After purification by column chromatography on silica gel then by reverse phase HPLC (acidic elution), the racemate was separated by chiral SFC (column Lux Cellulose-4 from Phenomenex, eluent 30% MeOH+0.03% NH₃, 70% CO₂) to yield the pure enantiomer as off-white solid (11 mg, yield: 4%) LC-MS (Method 2) m/z [M+H]⁺: 460.1, 462.1; rt: 2.13 min; purity: 99%. ¹H NMR (400 MHz, DMSO-d₆) δ 10.62 (s, 1H), 7.75 (dd, J=11.9, 2.4 Hz, 1H), 7.57-7.48 (m, 2H), 7.47-7.35 (m, 2H), 7.34-7.27 (m, 2H), 4.62-4.44 (m, 2H), 3.55 (q, J=6.7 Hz, 1H), 2.49 (s, 3H), 1.46 (d, J=6.8 Hz, 3H). ¹⁹F NMR (376 MHz, DMSO-d₆) δ−70.35, −114.71, −115.96. Chiral purity: 98.4%; rt=2.45 min (second eluting enantiomer). For information, first eluting enantiomer rt=2.15 min. Both measured by chiral SFC, Lux Cellulose-4 from Phenomenex, eluent 18% MeOH+0.1% NH₃, 82% CO₂.

### Example #406: Enantiomer (5S) or (5R) of N-(4-chloro-3-fluoro-phenyl)-2-[9,11-difluoro-5-methyl-6-oxo-5H-pyrido[2,3-d][1]benzazepin-7-yl]acetamide

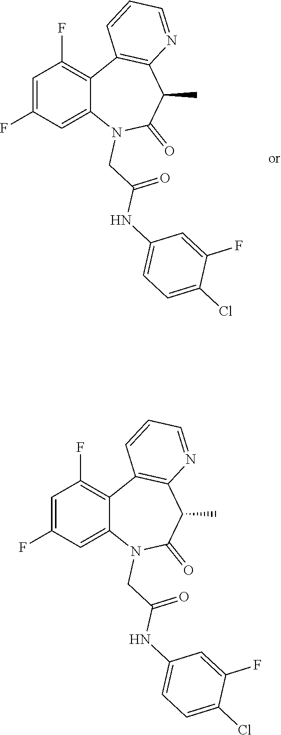 [SMILES: C[C@H]1C(=O)N(CC(=O)Nc2ccc(Cl)c(F)c2)c2cc(F)cc(F)c2-c2cccnc21 ; C[C@@H]1C(=O)N(CC(=O)Nc2ccc(Cl)c(F)c2)c2cc(F)cc(F)c2-c2cccnc21]

The title product was prepared according to the general procedure 1, starting from 9,11-difluoro-5-methyl-5,7-dihydropyrido[2,3-d][1]benzazepin-6-one NN358 (142 mg, 0.51 mmol) and 2-chloro-N-(4-chloro-3-fluoro-phenyl)acetamide (CAS 895641-02-8). After purification by column chromatography on silica gel, the racemate was separated by Chiral SFC (column CHIRALPAK IG from Daicel, eluant 35% MeOH, 65% CO₂) to yield the pure enantiomer (55 mg, yield: 39%). LC-MS m/z [M+H]⁺: 446.1, 448.1; purity: 99%. ¹H NMR (400 MHz, DMSO-d₆) δ 10.55 (s, 1H), 8.66 (dd, J=4.8, 1.7 Hz, 1H), 8.08 (ddd, J=7.9, 4.6, 1.7 Hz, 1H), 7.72 (dd, J=11.8, 2.4 Hz, 1H), 7.55-7.46 (m, 2H), 7.46-7.34 (m, 2H), 7.32-7.25 (m, 1H), 4.71-4.39 (m, 2H), 3.75 (q, J=6.6 Hz, 1H), 1.48 (d, J=6.6 Hz, 3H). ¹⁹F NMR (376 MHz, DMSO-d₆) δ−108.77 (d, J=8.1 Hz), −111.53 (d, J=8.1 Hz), −114.66 (s). Chiral purity: 99.6%; rt=2.80 min (second eluting enantiomer). For information, first eluting enantiomer rt=2.03 min. Both measured by HPLC, CHIRALPAK IG from Daicel, 35% MeOH+0.1% NH₃, 65% CO₂.

### Example #407: Enantiomer (8S) or (8R) of N-(4-chloro-3-fluoro-phenyl)-2-[8,13-dimethyl-9-oxo-3,10,14-triazatricyclo[9.4.0.0^{2,7}]pentadeca-1(15),2,4,6,11,13-hexaen-10-yl]acetamide

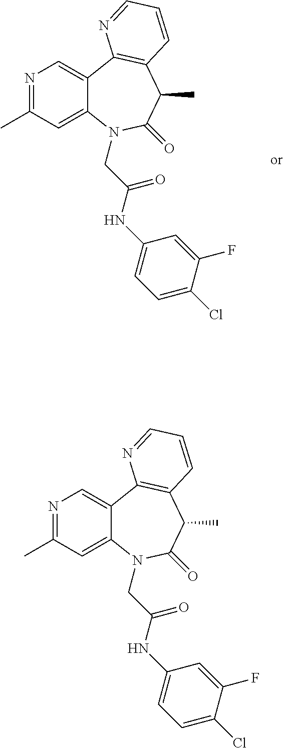 [SMILES: Cc1cc2c(cn1)-c1ncccc1[C@@H](C)C(=O)N2CC(=O)Nc1ccc(Cl)c(F)c1 ; Cc1cc2c(cn1)-c1ncccc1[C@H](C)C(=O)N2CC(=O)Nc1ccc(Cl)c(F)c1]

The title product was prepared according to the general procedure 1, starting from 8,13-dimethyl-3,10,14-triazatricyclo[9.4.0.0^{2,7}]pentadeca-1(11),2(7),3,5,12,14-hexaen-9-one NN359 (34.2 mg, 0.14 mmol) and 2-chloro-N-(4-chloro-3-fluoro-phenyl)acetamide (CAS 895641-02-8). The residue was purified by column chromatography on silica gel (using a gradient of 0-100% EtOAc-EtOH 3:1 in isohexane as eluent). The racemate was then separated by Chiral SFC (column Lux Cellulose-4 from Phenomenex, eluant 30% MeOH, 70% CO₂) to yield the pure enantiomer (13 mg, yield: 31%). LC-MS m/z [M+H]⁺: 425.1, 427.1; purity: 99%. ¹H NMR (400 MHz, DMSO-d₆) δ 10.58 (s, 1H), 8.94 (s, 1H), 8.70 (dd, J=4.8, 1.5 Hz, 1H), 7.85-7.80 (m, 1H), 7.75 (dd, J=11.9, 2.4 Hz, 1H), 7.58-7.53 (m, 1H), 7.51 (d, J=8.7 Hz, 1H), 7.34-7.28 (m, 2H), 4.68-4.43 (m, 2H), 3.52 (q, J=6.8 Hz, 1H), 2.56 (s, 3H), 1.48 (d, J=6.8 Hz, 3H). ¹⁹F NMR (376 MHz, DMSO-d₆) δ−114.67. Chiral purity: 99.4%; rt=3.26 min (Second eluting enantiomer). For information, first eluting enantiomer rt=2.67 min. Both measured by chiral SFC, Cellulose-4 Lux from Phenomenex, 30% MeOH+0.1% NH₃, 70% CO₂.

### Example #408: Enantiomer (8S) or (8R) of N-(4-chloro-3-fluoro-phenyl)-2-[15-fluoro-8,13-dimethyl-9-oxo-3,6,10,14-tetrazatricyclo[9.4.0.0^{2,7}]pentadeca-1(15),2,4,6,11,13-hexaen-10-yl]acetamide

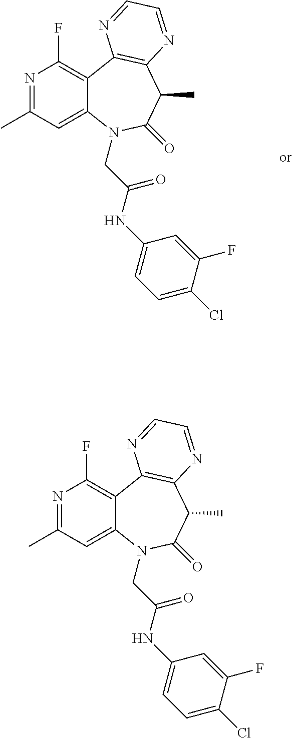 [SMILES: Cc1cc2c(c(F)n1)-c1nccnc1[C@@H](C)C(=O)N2CC(=O)Nc1ccc(Cl)c(F)c1 ; Cc1cc2c(c(F)n1)-c1nccnc1[C@H](C)C(=O)N2CC(=O)Nc1ccc(Cl)c(F)c1]

The title product was prepared according to the general procedure 1, starting from 15-fluoro-8,13-dimethyl-3,6,10,14-tetrazatricyclo[9.4.0.0^{2,7}]pentadeca-1(15),2,4,6,11,13-hexaen-9-one NN360 (65%, 270 mg, 0.68 mmol) and 2-chloro-N-(4-chloro-3-fluoro-phenyl)acetamide (CAS 895641-02-8). After purification by trituration with TBME/isohexane then by reverse phase HPLC (basic elution), the racemate was separated by chiral SFC (CHIRALPAK IG, eluent 45% MeOH+0.1% NH₃, 55% CO₂) to yield the pure enantiomer (29 mg, yield: 9%) LC-MS m/z [M+H]⁺: 444.0, 446.0; purity: 99%. H NMR (400 MHz, DMSO-d₆) δ 10.59 (s, 1H), 8.79-8.72 (m, 2H), 7.71 (dd, J=11.9, 2.4 Hz, 1H), 7.51 (t, J=8.7 Hz, 1H), 7.38 (s, 1H), 7.30 (ddd, J=8.8, 2.4, 1.0 Hz, 1H), 4.66-4.52 (m, 2H), 3.97 (q, J=6.5 Hz, 1H), 2.52 (s, 3H, DMSO overlap), 1.49 (d, J=6.6 Hz, 3H). ¹⁹F NMR (376 MHz, DMSO-d₆) δ−69.26, −114.66. Chiral purity: 100%; rt=3.51 min (second eluting enantiomer). For information, first eluting enantiomer rt=1.41 min. Both measured by chiral SFC, CHIRALPAK IG from Daicel, eluent 45% MeOH+0.1% NH₃, 55% CO₂.

### Example #409: Enantiomer (5S) or (5R) of N-(4-chloro-3-fluoro-phenyl)-2-[11-fluoro-5-methyl-6-oxo-5H-pyrido[2,3-d][1]benzazepin-7-yl]acetamide

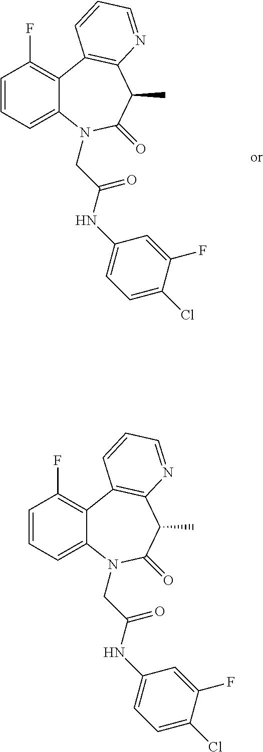 [SMILES: C[C@H]1C(=O)N(CC(=O)Nc2ccc(Cl)c(F)c2)c2cccc(F)c2-c2cccnc21 ; C[C@@H]1C(=O)N(CC(=O)Nc2ccc(Cl)c(F)c2)c2cccc(F)c2-c2cccnc21]

The title product was prepared according to the general procedure 1, starting from 11-fluoro-5-methyl-5,7-dihydropyrido[2,3-d][1]benzazepin-6-one NN361 (82%, 210 mg, 0.71 mmol) and 2-chloro-N-(4-chloro-3-fluoro-phenyl)acetamide (CAS 895641-02-8). The racemate was separated by Chiral SFC (column CHIRALPAK IC from Daicel, eluant 25% MeOH, 75% CO₂), followed by a further purification of both enantiomers by reverse phase HPLC (Basic) to yield the pure enantiomer as an off white solid (21 mg, yield: 21%). LC-MS m/z [M+H]⁺: 428.1/430.1; purity: 99%. ¹H NMR (400 MHz, DMSO-d₆) δ 10.52 (s, 1H), 8.66 (dd, J=4.8, 1.7 Hz, 1H), 8.09 (ddd, J=7.8, 4.6, 1.7 Hz, 1H), 7.72 (dd, J=11.9, 2.4 Hz, 1H), 7.58 (td, J=8.3, 6.3 Hz, 1H), 7.54-7.46 (m, 2H), 7.44 (dd, J=8.4, 1.2 Hz, 1H), 7.37-7.26 (m, 2H), 4.49 (d, J=2.1 Hz, 2H), 3.70 (q, J=6.6 Hz, 1H), 1.48 (d, J=6.7 Hz, 3H); ¹⁹F NMR (376 MHz, DMSO-d₆) δ−114.69, −115.73. Chiral purity: 99%; rt=3.05 min (second eluting enantiomer). For information, first eluting enantiomer rt=2.47 min. Both measured by SFC, CHIRALPAK IC from Daicel, 75% CO₂ and 25% MeOH+0.1% NH₃.

### Example #410: Enantiomer (10S) or (10R) of N-(4-chloro-3-fluoro-phenyl)-2-[14-fluoro-5-methoxy-10-methyl-9-oxo-4,8,12-triazatricyclo[9.4.0.0^{2,7}]pentadeca-1(15),2(7),3,5,11,13-hexaen-8-yl]acetamide

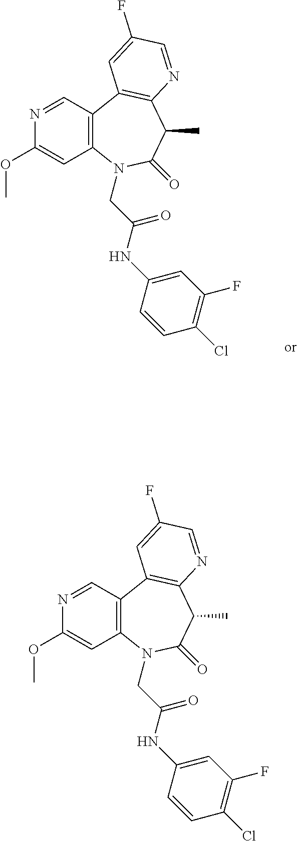 [SMILES: COc1cc2c(cn1)-c1cc(F)cnc1[C@@H](C)C(=O)N2CC(=O)Nc1ccc(Cl)c(F)c1 ; COc1cc2c(cn1)-c1cc(F)cnc1[C@H](C)C(=O)N2CC(=O)Nc1ccc(Cl)c(F)c1]

The title compound was prepared according to general procedure 1 starting from 14-fluoro-5-methoxy-10-methyl-4,8,12-triazatricyclo[9.4.0.0^{2,7}]pentadeca-1(11),2(7),3,5,12,14-hexaen-9-one N40B (80 mg, 0.30 mmol) and 2-chloro-N-(4-chloro-3-fluoro-phenyl)acetamide (85 mg, 0.38 mmol). The residue was purified by acidic basic preparative HPLC (Waters XBridge OBD MS C18 column using 0.5% TFA in water/MeCN (0 to 100%) as eluent) and basic preparative HPLC (Waters XBridge OBD MS C18 column using NH₄HCO₃ 10 mM+50 μl/L NH₄OH in water/MeCN (0 to 100%) as eluent) and then by liquid chiral chromatography (Chiralpak IG from Daicel, using Heptane-EtOH 1:1 as solvents) to give the compound as a white solid (9.2 mg, Yield: 7%) and the other enantiomer as white solid (8.7 mg, Yield=6.4%). LC-MS m/z [M+H]⁺: 459; purity: 99%. ¹H NMR (400 MHz, DMSO-d₆) δ 10.59 (s, 1H), 8.65 (d, J=2.7 Hz, 1H), 8.53 (s, 1H), 8.14 (dd, J=9.5, 2.8 Hz, 1H), 7.72 (dd, J=11.8, 2.4 Hz, 1H), 7.52 (t, J=8.7 Hz, 1H), 7.30 (dd, J=8.7, 2.3 Hz, 1H), 6.92 (s, 1H), 4.59 (d, J=3.4 Hz, 2H), 3.95 (s, 3H), 3.72 (q, J=6.5 Hz, 1H), 1.47 (d, J=6.6 Hz, 3H). Chiral purity: 100%; rt=2.66 min (second eluting enantiomer) column UHPLC ChiralPak IG with Heptane/EtOH 1:1+0.1% DEA as eluent. For information, first eluting enantiomer rt=1.75 min (100%). LC-MS (basic) m/z [M+H]⁺: 459; rt: 4.56 min; purity: 100%. LC-MS (acid) m/z [M+H]⁺: 459; rt: 4.86 min; purity: 100%.

### Example #411: Enantiomer (10S) or (10R) of 2-(3-chloro-5-methoxy-10-methyl-9-oxo-4,8,12-triazatricyclo[9.4.0.0^{2,7}]pentadeca-1(11),2(7),3,5,12,14-hexaen-8-yl]-N-[4-(difluoromethyl)phenyl)acetamide

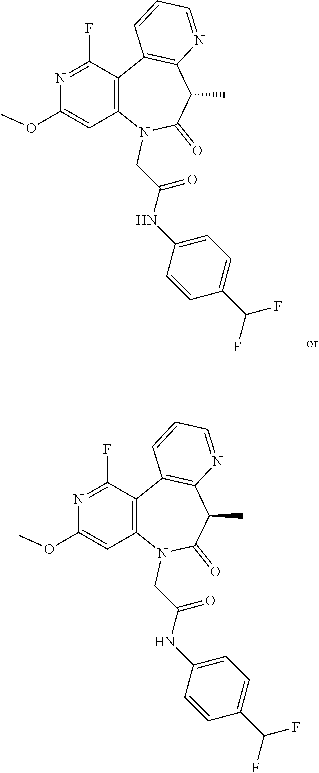 [SMILES: COc1cc2c(c(F)n1)-c1cccnc1[C@H](C)C(=O)N2CC(=O)Nc1ccc(C(F)F)cc1 ; COc1cc2c(c(F)n1)-c1cccnc1[C@@H](C)C(=O)N2CC(=O)Nc1ccc(C(F)F)cc1]

To a solution of 3-chloro-5-methoxy-10-methyl-4,8,12-triazatricyclo[9.4.0.0^{2,7}]pentadeca-1(11),2(7),3,5,12,14-hexaen-9-one NN171 (40.0 mg, 0.115 mmol), KI (10.0 mg, 0.060 mmol) and K2CO₃ (32.0 mg, 0.229 mmol) in DMF (0.6 mL) was added 2-chloro-N-[4-(difluoromethyl)phenyl]acetamide (30.0 mg, 0.137 mmol). The resulting mixture was stirred at room temperature for 3 h. Cold water (10 mL) was added to the reaction mixture and the precipitate formed was filtered-off, washed with cold water and dried to dryness. After purification by preparative HPLC (Purification Method P_B), the racemate residue was separated by chiral HPLC (Reprosil Chiral NR-R from Dr Maisch, eluant: iPrOH:Heptane 50%) to afford the title compound as a white solid (5.0 mg, yield: 28%). Chiral purity: 100%; rt=3.09 min (first eluting enantiomer). For information, second eluting enantiomer rt=4.88 min. Both measured by HPLC (Reprosil Chiral NR-R from Dr Maisch, eluant: iPrOH:Heptane 50%—DEA 0.1%). LC-MS m/z [M+H]⁺: 473.0/474.9; purity: 99%. ¹H NMR (400 MHz, DMSO-d₆) δ 10.45 (s, 1H), 8.63 (dd, J=4.8, 1.7 Hz, 1H), 8.20 (dd, J=8.0, 1.7 Hz, 1H), 7.67 (d, J=8.3 Hz, 2H), 7.53-7.44 (m, 3H), 7.00 (s, 1H), 6.96 (t, J=56.2 Hz, 1H), 4.64-4.48 (m, 2H), 3.94 (s, 3H), 3.87 (q, J=6.6 Hz, 1H), 1.47 (d, J=6.6 Hz, 3H).

### Example #412: N-(4-chloro-3-fluoro-phenyl)-2-[3-(difluoromethoxy)-10-methyl-9-oxo-4,8,12-triazatricyclo[9.4.0.0^{2,7}]pentadeca-1(15),2,4,6,11,13-hexaen-8-yl]acetamide

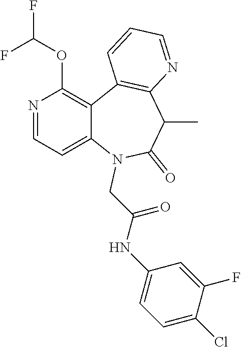 [SMILES: CC1C(=O)N(CC(=O)Nc2ccc(Cl)c(F)c2)c2ccnc(OC(F)F)c2-c2cccnc21]

### Step 1: Synthesis of ethyl 2-[3-(difluoromethoxy)-10-methyl-9-oxo-4,8,12-triazatricyclo[9.4.0.0^{2,7}]pentadeca-1(11),2,4,6,12,14-hexaen-8-yl]acetate #412_1

To a stirred solution of ethyl 2-(3-hydroxy-10-methyl-9-oxo-4,8,12-triazatricyclo[9.4.0.0^{2,7}]pentadeca-1(11),2,4,6,12,14-hexaen-8-yl)acetate NN441 (170 mg, 0.50 mmol) in DMF (7.00 mL) was added Cs₂CO₃ (164 mg, 0.50 mmol) and the reaction mixture was stirred for 30 minutes at 60° C. Then difluoro(iodo)methane (10% solution in THF, 2.52 mL, 1.26 mmol) was added and the reaction mixture was stirred at 60° C. for 2 h. The reaction mixture was diluted with water (180 mL) and extracted with EtOAc (3×30 mL). The combined organic extracts were washed with brine (60 mL), filtered through a hydrophobic frit and concentrated under reduced pressure. The crude residue was purified by column chromatography on silica gel (using a gradient, from 0 to 6% MeOH in DCM as eluant) to afford the title compound as an off white solid (75 mg, yield: 40%). LC-MS m/z [M+H]⁺: 378.1; purity: 97%. ¹H NMR (400 MHz, DMSO-d₆) δ 8.66 (dd, J=4.8, 1.7 Hz, 1H), 8.32 (d, J=5.8 Hz, 1H), 8.14 (dd, J=7.9, 1.7 Hz, 1H), 7.75 (t, J=144.6 Hz, 1H), 7.49 (dd, J=7.9, 4.7 Hz, 1H), 7.38 (d, J=5.8 Hz, 1H), 4.63-4.47 (m, 2H), 4.00 (q, J=7.1 Hz, 2H), 3.75 (q, J=6.6 Hz, 1H), 1.49 (d, J=6.6 Hz, 3H), 1.05 (t, J=7.1 Hz, 3H). ¹⁹F NMR (376 MHz, DMSO-d₆) δ−86.40 (d, J=172.8 Hz), −88.26 (d, J=172.7 Hz).

### Step 2: Synthesis of [N-(4-chloro-3-fluoro-phenyl)-2-[3-(difluoromethoxy)-10-methyl-9-oxo-4,8,12-triazatricyclo[9.4.0.0^{2,7}]pentadeca-1(11),2,4,6,12,14-hexaen-8-yl]acetamide #412

2-[3-(difluoromethoxy)-10-methyl-9-oxo-4,8,12-triazatricyclo[9.4.0.0^{2,7}]pentadeca-1(11),2,4,6,12,14-hexaen-8-yl]acetyl]oxylithium NN407 (68.5 mg, 0.19 mmol) prepared by saponification of #412_1 according to previously described procedures such as for intermediates N68-N72 was dissolved in DMF (5.0 mL) and 4-chloro-3-fluoro-aniline (56 mg, 0.39 mmol), N,N-diisopropylethylamine (0.13 mL, 0.77 mmol) and HATU (220 mg, 0.58 mmol) were sequentially added. The reaction mixture was stirred at RT for 4 h. Water (80 mL) was added and the aqueous phase extracted with EtOAc (2×20 mL). The combined organics were washed with brine (50 mL), then dried over anhydrous Na₂SO₄, filtered and concentrated under vacuo and the crude residue was purified by column chromatography on silica gel (using a gradient, 0-100% EtOAc in isohexanes as eluant) to afford the title compound as an off white solid (33 mg, yield: 46%). LC-MS m/z [M+H]⁺: 477.0, 479.0; purity: 97%. ¹H NMR (400 MHz, DMSO-d₆) δ 10.60 (s, 1H), 8.66 (dd, J=4.8, 1.7 Hz, 1H), 8.31 (d, J=5.7 Hz, 1H), 8.16 (dd, J=7.9, 1.7 Hz, 1H), 7.75 (t, J=72.3 Hz, 1H), 7.72 (dd, J=11.9, 2.3 Hz, 1H), 7.56-7.47 (m, 2H), 7.41 (d, J=5.8 Hz, 1H), 7.30 (ddd, J=8.8, 2.4, 1.0 Hz, 1H), 4.58 (s, 2H), 3.76 (q, J=6.5 Hz, 1H), 1.50 (d, J=6.6 Hz, 3H). ¹⁹F NMR (376 MHz, DMSO-d₆) δ−86.50 (d, J=172.8 Hz), −88.10 (d, J=172.6 Hz), −114.65.

### Example #413: Enantiomer (8S) or (8R) of N-(4-chloro-3-fluoro-phenyl)-2-[15-fluoro-13-methoxy-8-methyl-9-oxo-3,10,14-triazatricyclo[9.4.0.0^{2,7}]pentadeca-1(11),2,4,6,12,14-hexaen-10-yl]acetamide

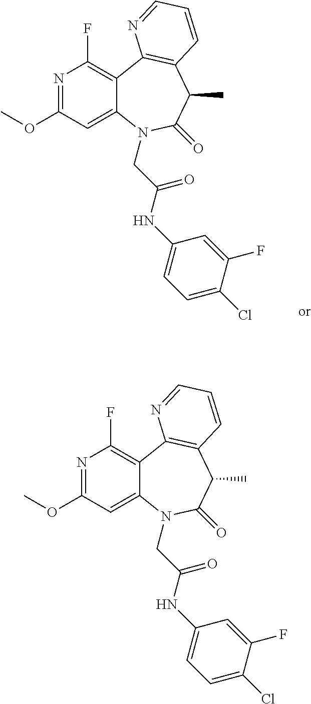 [SMILES: COc1cc2c(c(F)n1)-c1ncccc1[C@@H](C)C(=O)N2CC(=O)Nc1ccc(Cl)c(F)c1 ; COc1cc2c(c(F)n1)-c1ncccc1[C@H](C)C(=O)N2CC(=O)Nc1ccc(Cl)c(F)c1]

The title product was prepared according to the general procedure 1, starting from 15-fluoro-13-methoxy-8-methyl-3,10,14-triazatricyclo[9.4.0.0^{2,7}]pentadeca-1(11),2(7),3,5,12,14-hexaen-9-one NN362 (55%, 49 mg, 0.1 mmol) and 2-chloro-N-(4-chloro-3-fluoro-phenyl)acetamide (CAS 895641-02-8). After purification by column chromatography on silica gel (using a gradient of 0-100% EtOAc-EtOH 3:1 in isohexane as eluent) then by reverse phase HPLC (acidic elution), the racemate was separated by chiral SFC (column Lux Cellulose-4 from Phenomenex, eluent 30% MeOH, 70% CO₂) to yield the pure enantiomer (9 mg, yield: 14%) LC-MS m/z [M+H]⁺: 459.1, 461.1; purity: 100%. ¹H NMR (400 MHz, DMSO-d₆) δ 10.59 (s, 1H), 8.64 (dd, J=4.8, 1.5 Hz, 1H), 7.84-7.77 (m, 1H), 7.72 (dd, J=11.9, 2.4 Hz, 1H), 7.56-7.47 (m, 2H), 7.30 (ddd, J=8.8, 2.5, 1.0 Hz, 1H), 6.82 (s, 1H), 4.56 (s, 2H), 3.92 (s, 3H), 3.73 (q, J=6.8 Hz, 1H), 1.46 (d, J=6.8 Hz, 3H). ¹⁹F NMR (376 MHz, DMSO-d₆) δ−69.35, −114.63. Chiral purity: 100%; rt=2.21 min (second eluting enantiomer). For information, first eluting enantiomer rt=1.38 min. Both measured by chiral SFC, Lux Cellulose-4 from Phenomenex, eluent 40% MeOH+0.1% NH₃, 60% CO₂.

### Example #414: Enantiomer (5S) or (5R) of 2-[9-chloro-11-fluoro-5-methyl-6-oxo-5H-pyrazino[2,3-d][1]benzazepin-7-yl]-N-(4-chloro-3-fluoro-phenyl)acetamide

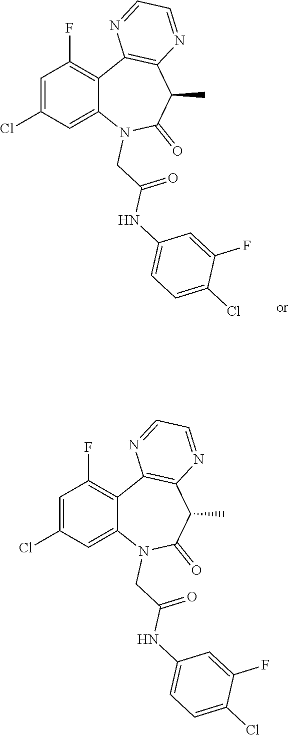 [SMILES: C[C@H]1C(=O)N(CC(=O)Nc2ccc(Cl)c(F)c2)c2cc(Cl)cc(F)c2-c2nccnc21 ; C[C@@H]1C(=O)N(CC(=O)Nc2ccc(Cl)c(F)c2)c2cc(Cl)cc(F)c2-c2nccnc21]

The title product was prepared according to the general procedure 1, starting from 9-chloro-11-fluoro-5-methyl-5,7-dihydropyrazino[2,3-d][1]benzazepin-6-one NN363 (143 mg, 0.510 mmol) and 2-chloro-N-(4-chloro-3-fluoro-phenyl)acetamide (CAS 895641-02-8). After purification by column chromatography on silica gel (using a gradient of 0-60% EtOAc in isohexane as eluent) the racemate was separated by chiral SFC (column CHIRALPAK A1 from Daicel, eluant 50% MeOH, 50% CO₂) affording the pure enantiomer as an off-white solid (90 mg, yield: 51%). LC-MS m/z [M+H]⁺: 463.1, 464.9; purity: 100%. ¹H NMR (400 MHz, DMSO-d₆) δ 10.55 (s, 1H), 8.79-8.72 (m, 2H), 7.69 (dd, J=11.9, 2.4 Hz, 1H), 7.59 (d, J=8.7 Hz, 2H), 7.51 (t, J=8.7 Hz, 1H), 7.32-7.25 (m, 1H), 4.64 (d, J=16.7 Hz, 1H), 4.48 (d, J=16.8 Hz, 1H), 3.94 (q, J=6.5 Hz, 1H), 1.48 (d, J=6.6 Hz, 3H). Chiral purity: 99%; rt=3.93 min (second eluting enantiomer). For information, first eluting enantiomer rt=1.10 min. Both measured by SFC, CHIRALPAK A1 from Daicel, MeOH 35%+0.1% NH₃, 65% CO₂.

### Example #415: Enantiomer (10S) or (10R) of N-(4-chloro-3-fluoro-phenyl)-2-[14-methoxy-5,10-dimethyl-9-oxo-4,8,12-triazatricyclo[9.4.0.0^{2,7}]pentadeca-1(15),2,4,6,11,13-hexaen-8-yl]acetamide

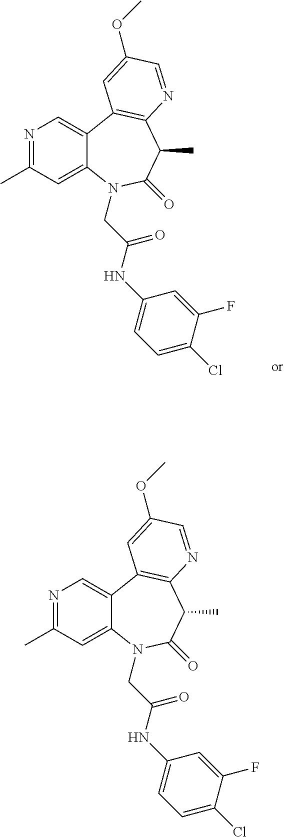 [SMILES: COc1cnc2c(c1)-c1cnc(C)cc1N(CC(=O)Nc1ccc(Cl)c(F)c1)C(=O)[C@@H]2C ; COc1cnc2c(c1)-c1cnc(C)cc1N(CC(=O)Nc1ccc(Cl)c(F)c1)C(=O)[C@H]2C]

The title product was prepared according to the general procedure 1, starting from 14-methoxy-5,10-dimethyl-4,8,12-triazatricyclo[9.4.0.0^{2,7}]pentadeca-1(11),2(7),3,5,12,14-hexaen-9-one NN364 (131 mg, 0.49 mmol) and 2-chloro-N-(4-chloro-3-fluoro-phenyl)acetamide (CAS 895641-02-8). The residue was purified by column chromatography on silica gel (using a gradient of 0-25% EtOH in EtOAc as eluent). The racemate was the separated by Chiral SFC (column A1 from Phenomenex, eluant 50% MeOH, 50% CO₂) to yield the pure enantiomer (50 mg, yield: 35%). LC-MS m/z [M+H]⁺: 455.2/457.1; purity: 100%. ¹H NMR (400 MHz, DMSO-d₆) δ 10.58 (s, 1H), 8.79 (s, 1H), 8.38 (d, J=2.8 Hz, 1H), 7.80-7.68 (m, 2H), 7.52 (t, J=8.7 Hz, 1H), 7.38-7.27 (m, 2H), 4.59 (d, J=16.8 Hz, 1H), 4.50 (d, J=16.8 Hz, 1H), 3.93 (s, 3H), 3.54 (q, J=6.6 Hz, 1H), 2.56 (s, 3H), 1.46 (d, J=6.7 Hz, 3H). Chiral purity: 99.3%; rt=4.27 min (Second eluting enantiomer). For information, first eluting enantiomer rt=1.24 min. Both measured by SFC, A1 from Phenomenex, 45% MeOH+0.1% NH₃, 55% CO₂.

### Example #416: Enantiomer (10S) or (10R) of 2-[3-chloro-5-(difluoromethoxy)-10-methyl-9-oxo-4,8,12-triazatricyclo[9.4.0.0^{2,7}]pentadeca-1(15),2,4,6,11,13-hexaen-8-yl]-N-[4-(difluoromethyl)phenyl]acetamide

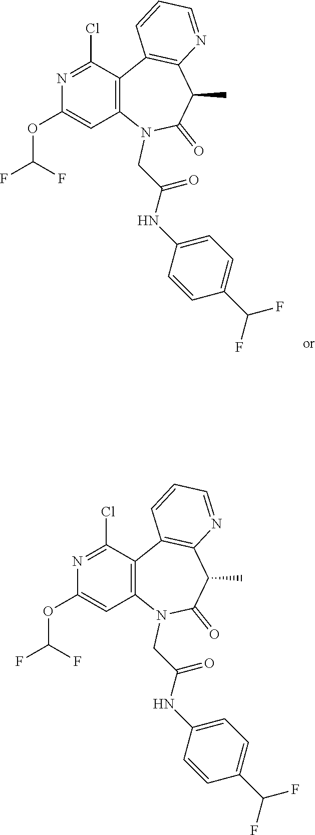 [SMILES: C[C@H]1C(=O)N(CC(=O)Nc2ccc(C(F)F)cc2)c2cc(OC(F)F)nc(Cl)c2-c2cccnc21 ; C[C@@H]1C(=O)N(CC(=O)Nc2ccc(C(F)F)cc2)c2cc(OC(F)F)nc(Cl)c2-c2cccnc21]

Under N₂ atmosphere, to a solution of 3-bromo-2-chloro-6-(difluoromethoxy)pyridin-4-amine NN175 (145 mg, 0.53 mmol), Ethyl 2-[3-(4,4,5,5-tetramethyl-1,3,2-dioxaborolan-2-yl)-2-pyridyl]propanoate N31_1 (211 mg, 0.69 mmol) and Pd[(amphos)CI]2 (38.5 mg, 0.05 mmol) in 1,4-Dioxane (4.3 mL) was added K2CO₃ (223 mg, 1.60 mmol) followed by water (1.1 mL). The reaction mixture was stirred at 100° C. for 3 h. After complete conversion, water (50 mL) was added and the reaction mixture was extracted with DCM (3×50 mL). The combined organic extracts were dried over MgSO₄, filtered, and concentrated under vacuum. The residue was dissolved in dry Toluene (2.7 mL) before addition of LiHMDS (1.5 M in THF, 1.06 mL, 1.57 mmol). The reaction mixture was stirred at room temperature for 3 h. After complete conversion, the reaction mixture was concentrated under vacuum. The residue was purified by flash chromatography on silica gel (using a gradient of 0 to 100% EtOAc in Heptane as eluent). The residue was dissolved in DMF (0.61 mL) before addition of K2CO₃ (34 mg, 0.24 mmol), KI (10 mg, 0.06 mmol) and 2-chloro-N-[4-(difluoromethyl)phenyl]acetamide (32 mg, 0.15 mmol). The reaction mixture was stirred at room temperature for 2 h. Water (10 mL) was added and the reaction mixture was extracted with DCM (3×10 mL). The combined organic extracts were dried over MgSO₄, filtered and concentrated under vacuum. After purification by preparative HPLC (Purification Method P_B), the racemate was separated by Chiral HPLC (Chiralpak IA from Daicel, EtOH 50%—heptane 50%) to afford the title compound (10.4 mg, yield: 4%) as a white solid. LC-MS m/z: [M+H]⁺: 509.1/511.1; purity: 99%. ¹H NMR (400 MHz, DMSO-d₆) δ 10.48 (s, 1H), 8.67 (dd, J=4.8, 1.7 Hz, 1H), 8.24 (dd, J=7.9, 1.7 Hz, 1H), 7.76 (t, J=72.0 Hz, 1H), 7.66 (d, J=8.4 Hz, 2H), 7.53-7.47 (m, 3H), 7.29 (s, 1H), 6.96 (t, J=56.1 Hz, 1H), 4.71-4.52 (m, 2H), 3.91 (q, J=6.5 Hz, 1H), 1.48 (d, J=6.5 Hz, 3H). ¹⁹F NMR (376 MHz, DMSO-d₆) δ−86.67-−88.06 (m), −108.08 (d, J=56.1 Hz). Chiral purity: 99%; rt=2.58 min (second eluting enantiomer). For information, first eluting enantiomer rt=1.60 min. Both measured by HPLC (Chiralpak IA from Daicel, EtOH 50%—heptane 50%—DEA 0.1%).

### Example #417: N-(4-chloro-3-fluoro-phenyl)-2-(5,15-difluoro-13-methyl-9-oxo-3,10,14-triazatricyclo[9.4.0.0^{2,7}]pentadeca-1(11),2,4,6,12,14-hexaen-10-yl)acetamide

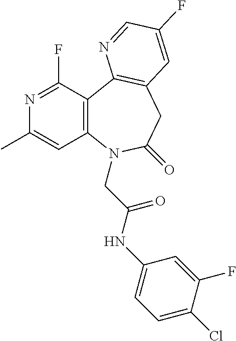 [SMILES: Cc1cc2c(c(F)n1)-c1ncc(F)cc1CC(=O)N2CC(=O)Nc1ccc(Cl)c(F)c1]

The title product was prepared according to general procedure 1, starting from 5,15-difluoro-13-methyl-3,10,14-triazatricyclo[9.4.0.0^{2,7}]pentadeca-1(11),2(7),3,5,12,14-hexaen-9-one NN365 (46%, 60.0 mg, 0.106 mmol) and 2-chloro-N-(4-chloro-3-fluoro-phenyl)acetamide (CAS 895641-02-8). Purification by column chromatography on silica gel (using a gradient of 0-100% EtOAc in isohexane as eluent) and reverse phase HPLC (acidic elution) afforded the title compound as a white solid (12 mg, yield: 25%). LC-MS m/z [M+H]⁺: 447.1, 449.1; purity: 99%. ¹H NMR (400 MHz, DMSO-d₆) δ 10.60 (s, 1H), 8.70 (d, J=2.8 Hz, 1H), 7.99 (dd, J=9.0, 2.8 Hz, 1H), 7.75 (dd, J=11.9, 2.4 Hz, 1H), 7.52 (t, J=8.7 Hz, 1H), 7.35-7.29 (m, 2H), 4.55 (s, 2H), 3.78-3.66 (m, 2H), 2.49 (d, J=1.2 Hz, 3H). ¹⁹F NMR (376 MHz, DMSO-d₆) δ−68.66, −114.63, −126.85.

### Example #418: Enantiomer (5S) or (5R) of 2-[9-chloro-11-fluoro-5-methyl-6-oxo-5H-pyrimido[5,4-d][1]benzazepin-7-yl]-N-(4-chloro-3-fluoro-phenyl)acetamide

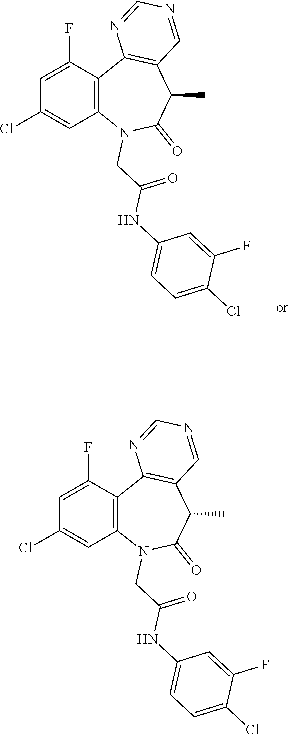 [SMILES: C[C@H]1C(=O)N(CC(=O)Nc2ccc(Cl)c(F)c2)c2cc(Cl)cc(F)c2-c2ncncc21 ; C[C@@H]1C(=O)N(CC(=O)Nc2ccc(Cl)c(F)c2)c2cc(Cl)cc(F)c2-c2ncncc21]

The title product was prepared according to the general procedure 1, starting from 9-chloro-11-fluoro-5-methyl-5,7-dihydropyrimido[5,4-d][1]benzazepin-6-one NN366 (121 mg, 0.087 mmol) and 2-chloro-N-(4-chloro-3-fluoro-phenyl)acetamide (CAS 895641-02-8). The crude was purified by column chromatography on silica gel (using a gradient of 0-100% EtOAc-EtOH 3:1 in isohexane as eluant) then by flash reverse phase chromatography on C18 silica gel (using a gradient of 0-100% MeCN in water as eluant). The racemate was then separated by chiral SFC (column CHIRALPAK IC from Daicel, eluant 30% MeOH+0.1% NH₃, 70% CO₂) affording the pure enantiomer as an off-white solid (29 mg, yield: 37%). LC-MS m/z [M+H]⁺: 463.1, 465.0; purity: 97%. ¹H NMR (400 MHz, DMSO-d₆) δ 10.54 (s, 1H), 9.28 (s, 1H), 8.88 (s, 1H), 7.68 (dd, J=11.8, 2.4 Hz, 1H), 7.60 (dd, J=10.1, 2.0 Hz, 1H), 7.57 (t, J=1.7 Hz, 1H), 7.51 (t, J=8.6 Hz, 1H), 7.29 (dt, J=9.0, 1.5 Hz, 1H), 4.63 (d, J=16.8 Hz, 1H), 4.47 (d, J=16.7 Hz, 1H), 3.75 (q, J=6.8 Hz, 1H), 1.53 (d, J=6.8 Hz, 3H). Chiral purity: 100%; rt=2.49 min (second eluting enantiomer). For information, first eluting enantiomer rt=1.93 min. Both measured by SFC, CHIRALPAK IC from Daicel, MeOH 30%+0.1% NH₃, 70% CO₂.

### Example #419: Enantiomer (10S) or (10R) of N-(4-chloro-3-fluoro-phenyl)-2-[3,14-difluoro-10-methyl-9-oxo-4,8,12-triazatricyclo[9.4.0.0^{2,7}]pentadeca-1(15),2,4,6,11,13-hexaen-8-yl]acetamide

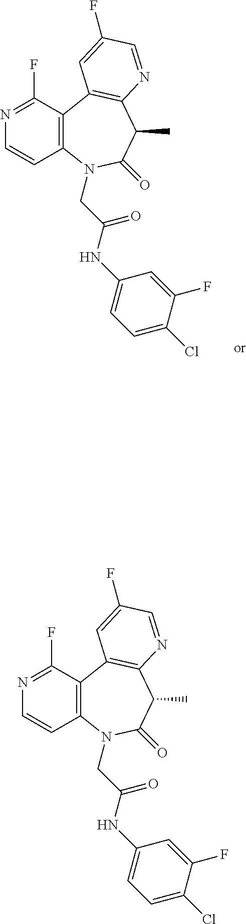 [SMILES: C[C@H]1C(=O)N(CC(=O)Nc2ccc(Cl)c(F)c2)c2ccnc(F)c2-c2cc(F)cnc21 ; C[C@@H]1C(=O)N(CC(=O)Nc2ccc(Cl)c(F)c2)c2ccnc(F)c2-c2cc(F)cnc21]

The title product was prepared according to the general procedure 1, starting from 3,14-difluoro-10-methyl-4,8,12-triazatricyclo[9.4.0.0^{2,7}]pentadeca-1(15),2(7),3,5,11,13-hexaen-9-one NN367 (100 mg, 0.38 mmol) and 2-chloro-N-(4-chloro-3-fluoro-phenyl)acetamide (CAS 895641-02-8). After purification by column chromatography on silica gel (using a gradient of 0-100% EtOAc in isohexane as eluent) then by reverse phase HPLC (acidic elution), the racemate was separated by chiral SFC (column CHIRALPAK IG from Daicel, eluent 40% MeOH, 60% CO₂) to yield the pure enantiomer (10 mg, yield: 9%) LC-MS m/z [M+H]⁺: 447.1, 449.1; purity: 100%. ¹H NMR (400 MHz, DMSO-de) δ 10.64 (s, 1H), 8.73 (d, J=2.8 Hz, 1H), 8.33 (d, J=5.7 Hz, 1H), 8.20 (dt, J=9.5, 3.5 Hz, 1H), 7.72 (dd, J=11.9, 2.4 Hz, 1H), 7.55-7.47 (m, 2H), 7.30 (dt, J=8.9, 1.6 Hz, 1H), 4.60 (d, J=3.4 Hz, 2H), 3.82 (q, J=6.5 Hz, 1H), 1.49 (d, J=6.6 Hz, 3H). ¹⁹F NMR (376 MHz, DMSO-d₆) δ−69.73, −114.69, −130.13. Chiral purity: 99.4%; rt=4.46 min (second eluting enantiomer). For information, first eluting enantiomer rt=1.60 min. Both measured by chiral SFC, CHIRALPAK IG from Daicel, eluent 30% MeOH+0.1% NH₃, 70% CO₂.

### Example #420: Enantiomer (5S) or (5R) of 2-[10-cyano-2-fluoro-5,9-dimethyl-6-oxo-5H-pyrido[2,3-d][1]benzazepin-7-yl]-N-[4-(difluoromethyl)phenyl]acetamide

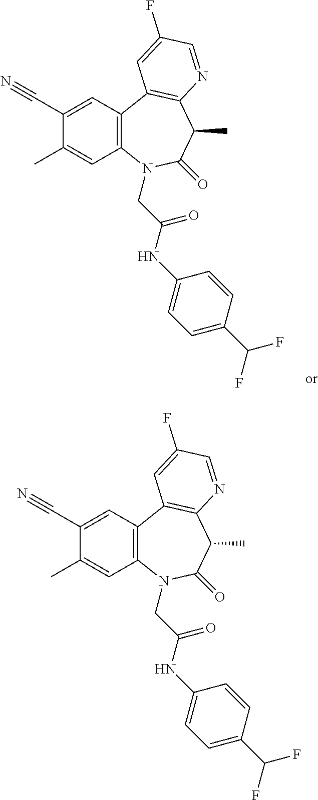 [SMILES: Cc1cc2c(cc1C#N)-c1cc(F)cnc1[C@@H](C)C(=O)N2CC(=O)Nc1ccc(C(F)F)cc1 ; Cc1cc2c(cc1C#N)-c1cc(F)cnc1[C@H](C)C(=O)N2CC(=O)Nc1ccc(C(F)F)cc1]

The title product was prepared according to the general procedure 1, starting from 2-fluoro-5,9-dimethyl-6-oxo-5,7-dihydropyrido[2,3-d][1]benzazepine-10-carbonitrile Intermediate NN321 (64 mg, 0.228 mmol) and 2-chloro-N-[4-(difluoromethyl)phenyl]acetamide (CAS 872533-93-2) (55 mg, 0.24 mmol). After purification by reverse phase HPLC (acidic elution), the racemate was separated by Chiral SFC (column CHIRALPAK IG from Daicel, eluant 45% MeOH+0.1% NH₃, 55% CO₂) to yield the pure enantiomer as a white solid (14.0 mg, yield: 40%). LC-MS m/z [M+H]⁺: 465.2; purity: 98%. ¹H NMR (400 MHz, DMSO-d₆) δ 10.43 (s, 1H), 8.68 (d, J=2.7 Hz, 1H), 8.24-8.18 (m, 2H), 7.72-7.64 (m, 3H), 7.56-7.48 (m, 2H), 7.03 (d, J=56.2 Hz, 1H), 4.58 (d, J=2.3 Hz, 2H), 3.70-3.61 (m, 1H), 2.57 (s, 3H), 1.47 (d, J=6.7 Hz, 3H). ¹⁹F NMR (376 MHz, DMSO-d₆) δ−108.10, −129.80 Chiral purity: 99.48%; rt=1.51 min (second eluting enantiomer). For information, first eluting enantiomer rt=1.13 min. Both measured by SFC, CHIRALPAK AY-H from Daicel, 45% MeOH+0.1% NH₃, 55% CO₂.

### Example #421: 2-[5-(2,2-difluoro-5-azaspiro[2.3]hexan-5-yl)-3-fluoro-9-oxo-4,8,12-triazatricyclo[9.4.0.0^{2,7}]pentadeca-1(11),2(7),3,5,12,14-hexaen-8-yl]-N-[4-(difluoromethyl)phenyl]acetamide

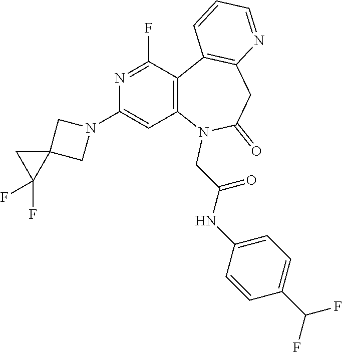 [SMILES: O=C(CN1C(=O)Cc2ncccc2-c2c1cc(N1CC3(C1)CC3(F)F)nc2F)Nc1ccc(C(F)F)cc1]

To a solution of N-[4-(difluoromethyl)phenyl]-2-(3,5-difluoro-9-oxo-4,8,12-triazatricyclo[9.4.0.0^{2,7}]pentadeca-1(11),2(7),3,5,12,14-hexaen-8-yl)acetamide NN191 (11 mg, 0.026 mmol) and 1,1-difluoro-5-azaspiro[2.3]hexane (4.6 mg, 0.035 mmol) in MeCN (0.2 mL) was added N,N-diisopropylethylamine (0.02 mL, 0.1 mmol) at room temperature and the reaction mixture was stirred at room temperature for 72 h. The reaction mixture was purified by preparative HPLC (using 0.3% NH₃ in water/MeCN 30 to 100% as eluent) to give the title compound (8 mg, yield: 53%) as a solid. LC-MS m/z [M+H]⁺: 530.1; purity: 90%.

### Example #422: 2-[3-chloro-5-(3-fluoro-1-methyl-azetidin-3-yl)-10-methyl-9-oxo-4,8,12-triazatricyclo[9.4.0.0^{2,7}]pentadeca-1(11),2,4,6,12,14-hexaen-8-yl]-N-(4-chloro-3-fluoro-phenyl)acetamide

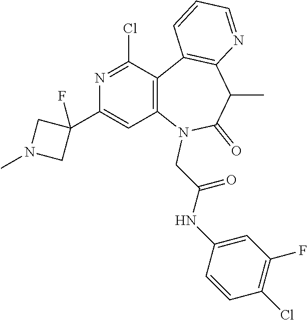 [SMILES: CC1C(=O)N(CC(=O)Nc2ccc(Cl)c(F)c2)c2cc(C3(F)CN(C)C3)nc(Cl)c2-c2cccnc21]

To a solution of tert-butyl 2-[3-chloro-5-(3-fluoro-1-methyl-azetidin-3-yl)-10-methyl-9-oxo-4,8,12-triazatricyclo[9.4.0.0^{2,7}]pentadeca-1(11),2,4,6,12,14-hexaen-8-yl]acetate NN153 (3.0 mg, 0.00637 mmol), in DCM (50.0 μL) was added, at room temperature, TFA (50 μL). The reaction mixture was stirred at room temperature for 1 h. After complete conversion, the reaction mixture was concentrated under reduced pressure. The residue was dissolved in DMF (100 μL) and 4-chloro-3-fluoroaniline (2.3 mg, 0.015 mmol), DIPEA (3.2 μL, 0.019 mmol) and HATU (4.8 mg, 0.013 mmol) were successively added at room temperature. The resulting mixture was stirred at RT for 2 h then purified by preparative HPLC (Purification Method P_B) to afford the title compound as a white solid (840 μg, yield: 25%). LC-MS m/z [M+H]⁺: 532.0/533.9; purity: 99%. ¹H NMR (600 MHz, DMSO-d₆) δ 10.59 (s, 1H), 8.69 (dd, J=4.7, 1.7 Hz, 1H), 8.29 (dd, J=7.9, 1.8 Hz, 1H), 7.73-7.67 (m, 2H), 7.55-7.50 (m, 2H), 7.30 (dd, J=8.9, 2.4 Hz, 1H), 4.69-4.49 (m, 2H), 3.87 (q, J=6.6 Hz, 1H), 3.81 (d, J=21.7 Hz, 2H), 3.68 (d, J=21.7 Hz, 2H), 2.43 (s, 3H), 1.48 (d, J=6.6 Hz, 3H).

### Example #423: Enantiomer (10S) or (10R) of 2-(3-chloro-14-fluoro-5,10-dimethyl-9-oxo-4,8,12-triazatricyclo[9.4.0.0^{2,7}]pentadeca-1(11),2,4,6,12,14-hexaen-8-yl)-N-[4-(1,1-difluoroethyl)phenyl]acetamide

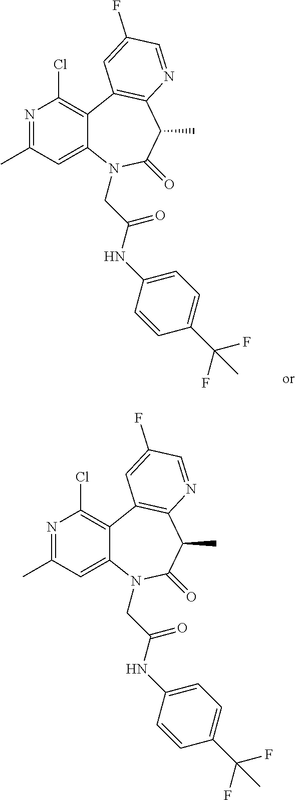 [SMILES: Cc1cc2c(c(Cl)n1)-c1cc(F)cnc1[C@H](C)C(=O)N2CC(=O)Nc1ccc(C(C)(F)F)cc1 ; Cc1cc2c(c(Cl)n1)-c1cc(F)cnc1[C@@H](C)C(=O)N2CC(=O)Nc1ccc(C(C)(F)F)cc1]

The title compound was prepared according to the general procedure 1, starting from 3-chloro-14-fluoro-5,10-dimethyl-4,8,12-triazatricyclo[9.4.0.0^{2,7}]pentadeca-1(11),2(7),3,5,12,14-hexaen-9-one (Intermediate NN444, 100 mg, 0.26 mmol) and 2-chloro-N-[4-(1,1-difluoroethyl)phenyl]acetamide (CAS 872533-93-2, 74.5 mg, 0.32 mmol). After purification by preparative HPLC (Method P_B), the racemate was separated by Chiral HPLC (Chiralpak AD from Daicel, iPrOH 50%—heptane 50%) to afford the title compound as a white solid (27.8 mg, yield: 21%). LC-MS m/z: [M+H]⁺: 489.1/491.1; purity: 100%. ¹H NMR (400 MHz, DMSO-d₆) δ 10.42 (s, 1H), 8.70 (d, J=2.8 Hz, 1H), 8.24 (dd, J=9.7, 2.8 Hz, 1H), 7.64 (d, J=8.4 Hz, 2H), 7.50 (d, J=8.4 Hz, 2H), 7.47 (s, 1H), 4.63-4.47 (m, 2H), 3.83 (q, J=6.6 Hz, 1H), 2.53 (s, 3H), 1.94 (t, J=18.7 Hz, 3H), 1.47 (d, J=6.6 Hz, 3H). ¹⁹F NMR (376 MHz, DMSO) δ−83.10 (q, J=18.7 Hz), −130.39 (d, J=9.7 Hz). Chiral purity: 100%; rt=2.14 min (second eluting enantiomer). For information, first eluting enantiomer rt=1.46 min. Both measured by HPLC (Chiralpak AD from Daicel, iPrOH 50%—heptane 50%—DEA 0.1%).

### Example #424: Enantiomer (10S) or (10R) of 2-(3-chloro-14-fluoro-5,10-dimethyl-9-oxo-4,8,12-triazatricyclo[9.4.0.0^{2,7}]pentadeca-1(11),2,4,6,12,14-hexaen-8-yl)-N-[5-(trifluoromethyl)-2-pyridyl]acetamide

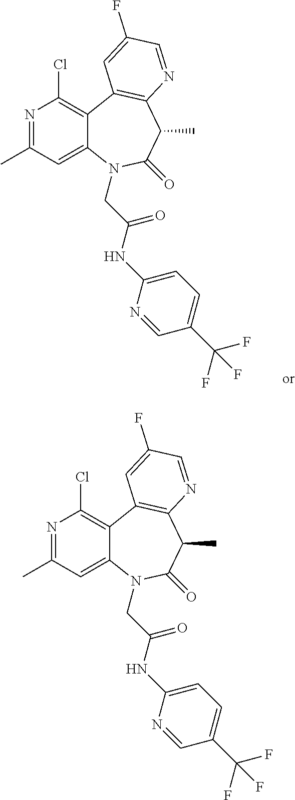 [SMILES: Cc1cc2c(c(Cl)n1)-c1cc(F)cnc1[C@H](C)C(=O)N2CC(=O)Nc1ccc(C(F)(F)F)cn1 ; Cc1cc2c(c(Cl)n1)-c1cc(F)cnc1[C@@H](C)C(=O)N2CC(=O)Nc1ccc(C(F)(F)F)cn1]

The title compound was prepared according to the general procedure 1, starting from 3-chloro-14-fluoro-5,10-dimethyl-4,8,12-triazatricyclo[9.4.0.0^{2,7}]pentadeca-1(11),2(7),3,5,12,14-hexaen-9-one (Intermediate NN444, 100 mg, 0.26 mmol) and 2-chloro-N-[5-(trifluoromethyl)-2-pyridyl]acetamide (Intermediate NN194, 82.9 mg, 0.31 mmol). After purification by preparative HPLC (Method P_B), the racemate was separated by Chiral HPLC (Whelk 0-1 (R,R) from Regis Technology, iPrOH 50%—heptane 50%) to afford the title compound as a white solid (32.1 mg, yield: 25%). LC-MS m/z: [M+H]⁺: 494.1/496.1; purity: 99%. ¹H NMR (400 MHz, DMSO-d₆) δ 11.24 (s, 1H), 8.73 (s, 1H), 8.70 (d, J=2.8 Hz, 1H), 8.23 (dd, J=9.7, 2.8 Hz, 1H), 8.19-8.16 (m, 2H), 7.46 (s, 1H), 4.63 (s, 2H), 3.84 (q, J=6.6 Hz, 1H), 2.53 (s, 3H), 1.46 (d, J=6.6 Hz, 3H). ¹⁹F NMR (376 MHz, DMSO) δ−60.20, −130.34 (d, J=9.7 Hz). Chiral purity: 100%; rt=2.38 min (first eluting enantiomer). For information, second eluting enantiomer rt=3.21 min. Both measured by HPLC (Whelk 0-1 (R,R) from Regis Technology, iPrOH 50%—heptane 50%—DEA 0.1%).

### V. Biological Assays

The effect of compounds of Formula (I) on the system Xc⁻ functionality was examined in two independent cell assays: (i) measurement of cystine-induced glutamate release; and (ii) measurement of [¹⁴C]L-Cystine uptake.

As described above, System Xc⁻, also known as the cystine/glutamate antiporter, is an amino acid transporter that mediates the extrusion of intracellular L-glutamate outside the cell and the uptake of extracellular L-cystine into the cell.

By measuring respectively, the concentration of glutamate released outside the cell and the L-cystine uptaken in the cell, with increasing concentrations of compounds of formula (I) according to the present invention, the efficacy of said compounds to inhibit the System Xc⁻ function can be evaluated.

The efficacy of the compounds to inhibit system Xc⁻ is represented by measuring the IC₅₀ which corresponds to the concentration of compound necessary to inhibit 50% of the signals from the two control groups; DMSO 1% and Erastin 50 μM in the respective assays. pIC₅₀ values correspond to −log of the IC₅₀ in Molar.

The lower the value of the IC₅₀ (the higher the value of the pIC₅₀ is), the less compound is needed to perform the same amount of inhibition and therefore the higher is the inhibition potency.

Generally, inhibitors of system Xc⁻ function will display values of IC₅₀ of 500 nM or lower respectively in each of the cystine-induced glutamate release and [¹⁴C]L-Cystine uptake assays.

When tested in each of the cystine-induced glutamate release and [¹⁴C]L-Cystine uptake assays, compounds of formula (I) according to the present invention display values of pIC₅₀ generally greater than or equal to about 6.3, suitably greater than about 7.0, ideally greater than about 7.4, appositely greater than about 7.8.

### V.1. Cystine-Induced Glutamate Release Assay

Measurement of cystine-induced glutamate release indicates levels of glutamate (outside of the cell) that are dependent on system Xc⁻ antiporter function. Upon the addition of L-Cystine to H4 cells, intracellular glutamate is counter-transported and exported in the supernatant.

In this assay, the medium is devoid of sodium to prevent the transport of glutamate by sodium-dependent excitatory amino acid transporters (EAATs). H4 cells were plated on 384-well culture plate at 1×10⁴ cells/well in Dulbecco's Modified Eagle Medium (DMEM) with L-glutamine, penicillin/streptomycin and 10% fetal bovine serum (all from ThermoFisher Scientific) and incubated in a humidified CO₂ incubator at 37° C. After 2 days, cells were washed 4 times with prewarmed sodium-free HEPES buffer (contents in mM: HEPES 10, KCl 5.4, CaCl₂ 2.5, MgCl₂ 1, KH₂PO₄ 0.4, D-Glucose 5, Choline chloride 140), pH 7.4. Glutamate release was induced by addition of 50 μM of L-Cystine (Sigma-Aldrich), together with a test compound of Formula (I) at 1% final DMSO concentration. After 2 hours of incubation in the culture incubator, supernatants were transferred to a low-binding polypropylene 384-well plate to determine L-glutamate concentration using Amplex™ Red Glutamic Acid Assay Kit (ThermoFisher Scientific). Briefly, 10 μL of Amplex™ Red working solution was added to 10 μL of the supernatant, then the mixture was incubated for 30 min at 37° C. Fluorescence intensity was measured by EnVision microplate reader (PerkinElmer).

When tested, compounds of formula (I) according to the present invention have displayed a PlC50 of about 6.3 or greater in the cystine-induced glutamate release assay.

### V.2. [¹⁴C]L-Cystine Uptake Assay

In this assay, the medium is devoid of sodium to prevent the transport of glutamate by sodium-dependent excitatory amino acid transporters (EAATs). H4 cells were plated on a 96-well CytoStar-T scintillating microplate (PerkinElmer) at 5×10⁴ cells/well in the medium described above. After 1 day, cells were washed one time with the sodium-free HEPES buffer and preincubated with test compounds of Formula (I) (1% final DMSO) for 15 min. Uptake was initiated by adding 0.5 μM L-[1,2, 1′,2′-¹⁴C]Cystine (0.02 mCi/mL, PerkinElmer, Waltham, US) and 4.5 μM L-Cystine (Sigma-Aldrich). After 1 hour at 37° C., plates were counted in a MicroBeta² microplate counter (PerkinElmer).

TABLE I
exhibits the ranges of pIC₅₀ of the compounds of
formula (I) according to the present invention when
tested in the Cystine-induced glutamate release assay
and/or [14C] L-Cystine uptake assay.

| | Cystine- | [¹⁴C] L- |
|---|---|---|
| | induced | Cystine |
| | glutamate | uptake |
| Example | release assay | assay |
| #1 | A | B |
| #2 | A | C |
| #3 | A | B |
| #4 | A | A |
| #5 | A | B |
| #6 | A | A |
| #7 | A | B |
| #8 | A | B |
| #9 | A | B |
| #10 | A | A |
| #11 | A | A |
| #12 | A | A |
| #13 | A | B |
| #14 | A | D |
| #15 | A | C |
| #16 | A | A |
| #17 | A | A |
| #18 | A | A |
| #19 | A | A |
| #21 | A | B |
| #22 | A | B |
| #23 | B | C |
| #24 | A | C |
| #25 | A | A |
| #26 | A | B |
| #27 | C | D |
| #28 | B | B |
| #29 | A | A |
| #30 | A | D |
| #31 | A | C |
| #32 | A | A |
| #33 | B | C |
| #34 | A | B |
| #35 | B | C |
| #36 | B | A |
| #37 | A | A |
| #38 | A | A |
| #39 | C | C |
| #40 | C | D |
| #41 | A | A |
| #42 | B | B |
| #43 | B | A |
| #44 | B | B |
| #45 | B | A |
| #46 | B | C |
| #47 | B | C |
| #48 | A | A |
| #49 | C | D |
| #50 | B | D |
| #51 | B | D |
| #52 | A | A |
| #53 | A | B |
| #54 | A | B |
| #55 | B | A |
| #56 | B | B |
| #57 | C | C |
| #58 | B | A |
| #59 | A | A |
| #60 | A | A |
| #61 | C | D |
| #62 | C | D |
| #63 | C | D |
| #64 | C | D |
| #65 | C | D |
| #66 | A | B |
| #67 | B | A |
| #68 | B | B |
| #69 | B | C |
| #70 | B | n.t. |
| #71 | A | A |
| #72 | B | C |
| #73 | A | A |
| #74 | A | A |
| #75 | C | C |
| #76 | A | A |
| #77 | B | A |
| #78 | A | A |
| #79 | A | A |
| #80 | C | C |
| #81 | C | D |
| #82 | A | B |
| #83 | D | D |
| #84 | C | D |
| #85 | B | A |
| #86 | B | C |
| #87 | A | B |
| #88 | B | B |
| #89 | B | A |
| #90 | C | D |
| #91 | B | C |
| #92 | B | D |
| #93 | B | C |
| #94 | B | B |
| #95 | C | B |
| #96 | C | C |
| #97 | B | D |
| #98 | B | C |
| #99 | B | A |
| #100 | B | B |
| #101 | C | D |
| #102 | A | B |
| #103 | A | A |
| #104 | B | B |
| #105 | B | B |
| #106 | B | A |
| #107 | C | D |
| #108 | B | D |
| #109 | B | C |
| #110 | A | B |
| #111 | A | B |
| #112 | B | D |
| #113 | B | D |
| #114 | C | D |
| #115 | A | B |
| #116 | D | D |
| #117 | D | D |
| #118 | A | B |
| #119 | C | C |
| #120 | C | D |
| #121 | D | D |
| #122 | A | B |
| #123 | A | B |
| #124 | B | C |
| #125 | B | B |
| #126 | C | D |
| #127 | B | D |
| #128 | C | D |
| #129 | B | B |
| #130 | B | B |
| #131 | B | C |
| #132 | B | D |
| #133 | B | B |
| #134 | B | D |
| #135 | C | D |
| #136 | A | C |
| #137 | C | C |
| #138 | D | D |
| #139 | C | C |
| #140 | C | C |
| #141 | C | C |
| #142 | C | D |
| #143 | C | D |
| #144 | C | D |
| #145 | D | D |
| #146 | B | A |
| #147 | B | A |
| #148 | B | C |
| #149 | C | D |
| #150 | B | C |
| #151 | D | D |
| #152 | B | C |
| #153 | C | C |
| #154 | C | D |
| #155 | D | D |
| #156 | C | B |
| #157 | C | D |
| #158 | C | D |
| #159 | B | C |
| #160 | A | C |
| #161 | C | C |
| #162 | C | C |
| #163 | C | C |
| #164 | A | B |
| #165 | A | A |
| #166 | C | C |
| #167 | B | C |
| #168 | A | C |
| #169 | A | C |
| #170 | B | B |
| #171 | B | C |
| #172 | C | C |
| #173 | C | D |
| #174 | D | D |
| #175 | D | D |
| #176 | D | D |
| #177 | D | D |
| #178 | D | D |
| #179 | B | C |
| #180 | B | C |
| #181 | C | D |
| #182 | C | C |
| #183 | A | B |
| #184 | C | D |
| #185 | B | B |
| #186 | C | B |
| #187 | B | C |
| #188 | D | D |
| #189 | D | D |
| #190 | A | B |
| #191 | A | B |
| #192 | D | D |
| #193 | C | D |
| #194 | C | D |
| #195 | D | D |
| #196 | C | D |
| #197 | D | D |
| #198 | B | B |
| #199 | C | D |
| #200 | C | D |
| #201 | C | D |
| #202 | B | C |
| #203 | C | D |
| #204 | B | D |
| #205 | C | D |
| #206 | B | D |
| #207 | C | D |
| #208 | D | D |
| #209 | C | C |
| #210 | C | D |
| #211 | D | C |
| #212 | A | C |
| #213 | C | C |
| #214 | D | C |
| #215 | C | C |
| #216 | B | C |
| #217 | D | D |
| #218 | C | A |
| #219 | D | D |
| #220 | D | D |
| #221 | B | A |
| #222 | C | B |
| #223 | D | C |
| #224 | C | D |
| #225 | D | D |
| #226 | D | C |
| #227 | D | C |
| #228 | D | D |
| #229 | C | B |
| #230 | C | C |
| #233 | B | C |
| #234 | C | D |
| #235 | D | D |
| #236 | C | C |
| #237 | C | B |
| #238 | B | B |
| #239 | C | C |
| #240 | C | B |
| #241 | D | C |
| #242 | C | B |
| #243 | C | B |
| #244 | C | C |
| #245 | C | n.t. |
| #246 | D | D |
| #247 | B | B |
| #248 | D | D |
| #249 | B | A |
| #250 | C | B |
| #251 | D | D |
| #252 | D | C |
| #253 | B | B |
| #254 | D | D |
| #256 | D | D |
| #257 | B | B |
| #258 | D | D |
| #259 | D | D |
| #260 | D | D |
| #261 | D | D |
| #262 | C | C |
| #263 | D | D |
| #264 | C | D |
| #265 | D | D |
| #266 | C | C |
| #267 | C | B |
| #268 | D | D |
| #269 | B | B |
| #270 | B | B |
| #271 | C | D |
| #272 | C | C |
| #273 | D | D |
| #274 | D | D |
| #275 | D | D |
| #276 | D | D |
| #277 | C | C |
| #278 | B | C |
| #279 | D | D |
| #280 | D | D |
| #281 | B | B |
| #282 | C | D |
| #283 | D | C |
| #284 | D | D |
| #285 | D | D |
| #286 | D | D |
| #287 | C | D |
| #288 | D | D |
| #289 | C | D |
| #290 | B | C |
| #291 | D | C |
| #292 | D | n.t. |
| #293 | B | C |
| #294 | C | B |
| #295 | D | D |
| #296 | B | C |
| #297 | D | D |
| #298 | D | D |
| #299 | D | D |
| #300 | D | D |
| #301 | B | B |
| #302 | B | C |
| #303 | D | C |
| #304 | C | C |
| #305 | B | n.t. |
| #306 | C | B |
| #307 | D | C |
| #308 | D | C |
| #309 | C | C |
| #310 | D | D |
| #311 | D | B |
| #312 | D | B |
| #313 | D | C |
| #314 | C | D |
| #315 | D | D |
| #316 | C | C |
| #317 | D | C |
| #318 | D | n.t. |
| #319 | C | B |
| #320 | C | C |
| #321 | C | C |
| #322 | C | B |
| #323 | D | D |
| #324 | D | D |
| #325 | C | D |
| #326 | D | D |
| #327 | D | D |
| #328 | D | C |
| #329 | C | C |
| #330 | C | D |
| #331 | B | C |
| #332 | C | D |
| #333 | B | C |
| #334 | D | D |
| #335 | D | D |
| #336 | C | B |
| #337 | C | C |
| #338 | D | n.t. |
| #339 | D | D |
| #340 | B | n.t. |
| #341 | C | B |
| #342 | D | D |
| #343 | B | A |
| #344 | B | A |
| #345 | C | C |
| #346 | B | B |
| #347 | B | C |
| #348 | C | D |
| #349 | C | D |
| #350 | C | C |
| #351 | B | B |
| #352 | C | D |
| #353 | C | B |
| #354 | C | D |
| #355 | C | B |
| #356 | B | B |
| #357 | C | C |
| #358 | C | A |
| #359 | C | C |
| #360 | C | B |
| #361 | C | D |
| #362 | C | D |
| #363 | C | B |
| #364 | C | D |
| #365 | D | C |
| #366 | B | B |
| #367 | C | C |
| #368 | D | D |
| #369 | C | C |
| #370 | B | B |
| #371 | D | D |
| #372 | D | C |
| #373 | C | C |
| #374 | D | D |
| #375 | D | D |
| #376 | C | B |
| #378 | C | B |
| #379 | C | D |
| #380 | D | D |
| #381 | B | A |
| #382 | C | C |
| #383 | B | C |
| #384 | D | D |
| #385 | C | D |
| #386 | D | D |
| #387 | C | C |
| #388 | C | C |
| #389 | D | D |
| #390 | C | C |
| #391 | A | C |
| #392 | B | C |
| #393 | B | C |
| #394 | C | B |
| #395 | C | B |
| #396 | C | C |
| #397 | B | B |
| #398 | C | B |
| #399 | B | C |
| #400 | B | A |
| #401 | D | D |
| #402 | D | D |
| #403 | D | D |
| #404 | C | B |
| #405 | C | D |
| #406 | C | C |
| #407 | B | B |
| #408 | B | B |
| #409 | C | C |
| #410 | C | C |
| #411 | D | D |
| #412 | B | B |
| #413 | B | C |
| #414 | B | C |
| #415 | D | D |
| #416 | C | D |
| #417 | B | C |
| #418 | B | B |
| #419 | C | D |
| #420 | D | D |
| #421 | C | C |
| #422 | C | n.t. |
| #423 | D | D |
| #424 | C | C |

Category A: about 6.3 ≤ pIC₅₀ ≤ about 7.00;
Category B: about 7.00 < pIC₅₀ ≤ about 7.40;
Category C: about 7.40 < pIC₅₀ ≤ about 7.80;
Category D: plC₅₀ > about 7.80.
n.t.: not tested.

As shown in this Table I, compounds of formula (I) according to the present invention are potent inhibitors of the System Xc⁻ function.

1. A compound of formula (I) or a pharmaceutically acceptable salt thereof, 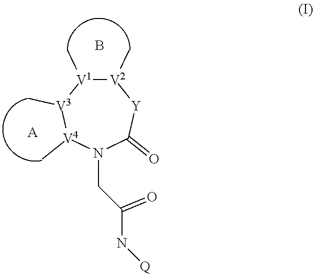
Wherein
Y represents N—R^{a} or CR^{1a}R^{1b};
Ra represents hydrogen or C₁₋₄ alkyl;
R^{1a} and R^{1b} represent independently hydrogen, hydroxy, halogen; or C₁₋₄ alkoxy or C₁₋₄ alkyl, either of which groups may be optionally substituted with one or more substituents; and
A, together with the points of attachment to the remainder of the molecule, V³ and V⁴, represents an optionally substituted aryl or heteroaryl selected from the groups represented by A¹, A², A³, A⁴, A⁵, A⁶ and A⁷: 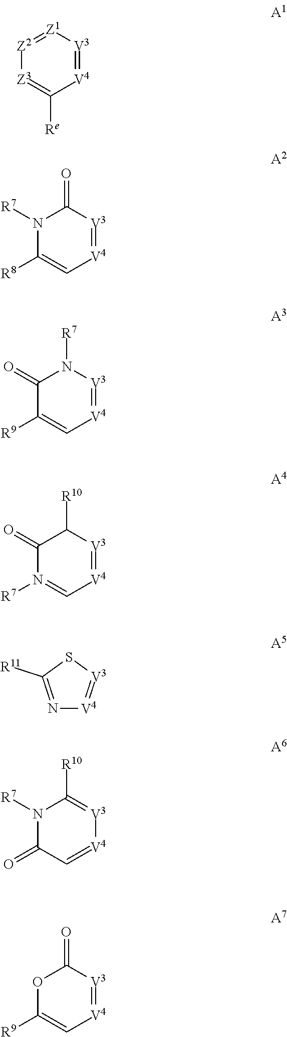
Wherein
V³ and V⁴ represent independently C;
Z¹ represents N or C—R⁴;
Z² represents N or C—R⁵;
Z³ represents N or C—R⁶;
R^{e} represents hydrogen or halogen;
R⁴ represents hydrogen, halogen, hydroxy, cyano or amino; or C₁₋₄ alkyl, C₃₋₇ cycloalkyl or C₁₋₄ alkoxy, any of which groups may be optionally substituted by one or more substituents;
R⁵ represents hydrogen, halogen or cyano; or C₁₋₄ alkyl or C₁₋₄ alkoxy, either of which groups may be optionally substituted by one or more substituents;
R⁶ represents hydrogen, halogen, or cyano; or C₁₋₄ alkoxy, C₁₋₄ alkylamino, C₁₋₄ alkyl, C₃₋₇ heterocycloalkyl or —O—(C₃₋₇ heterocycloalkyl), any of which groups may be optionally substituted by one or more substituents;
R⁷ represents hydrogen; or C₁₋₄ alkyl or C₃₋₇ cycloalkyl, either of which groups may be optionally substituted by one or more substituents;
R⁸, R⁹ and R¹⁰ independently represent hydrogen or halogen; or C₁₋₄ alkyl, C₃₋₇ cycloalkyl or C₁₋₄ alkoxy, any of which groups may be optionally substituted by one or more substituents;
R¹¹ represents —NR^{c}—(CO)—R^{b};
R^{b} and R^{c} represent independently C₁₋₄ alkyl; and
B, together with the points of attachment to the remainder of the molecule, V¹ and V², represents an optionally substituted aryl or heteroaryl selected from the groups represented by B¹, B², B³, B⁴, B⁵, B⁶, B⁷ and B⁸: 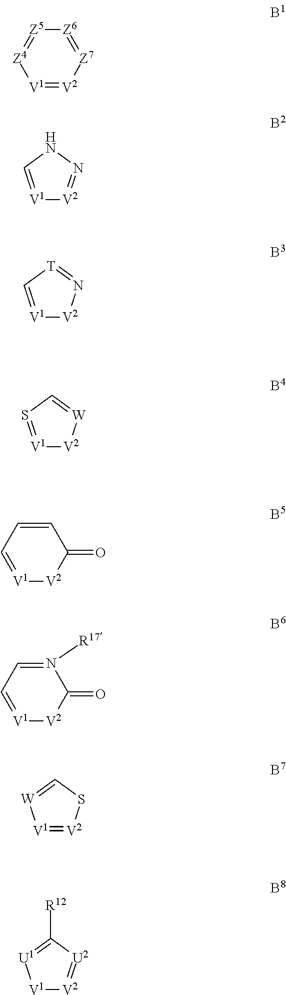
Wherein
V¹ represents C for B¹, B², B³, B⁴, B⁵, B⁶ and B⁷ and represents N for B⁸;
V² represents C for B¹, B², B⁴, B⁶, B⁷, and B⁸ and represents N for B³ and B⁵;
W, U¹ and U² represent independently N or C—H;
Z⁴ represents N or C—R¹³;
Z⁵ represents N or C—R¹⁴;
Z⁶ represents N or C—R¹⁵;
Z⁷ represents N or C—R¹⁶;
T represents N or C—R¹⁷;
R¹² represents hydrogen;
R¹³, R¹⁴, R¹⁵, R¹⁶ represent independently hydrogen, halogen, or cyano; or C₁₋₄ alkyl or C₁₋₄ alkoxy, either of these groups which may be optionally substituted by one or more substituents;
R¹⁷ represents hydrogen, halogen or C₁₋₄ alkyl;
R¹⁷′ represents hydrogen or C₁₋₄ alkyl; and
Q represents a ring selected from the groups represented by Q¹ and Q²: 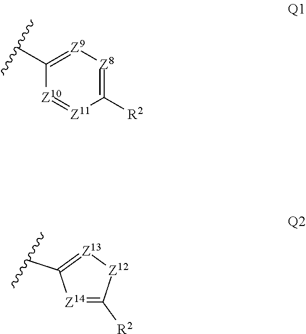
Wherein
Z⁸ represents N or C—R³;
Z⁹ represents C—R¹⁸;
Z¹⁰ represents N or C—R¹⁹;
Z¹¹ represents C—R²⁰;
Z¹² represents S, O, N—H or CR²¹R²²;
Z¹³ represents N or C—R²³;
Z¹⁴ represents N or C—R²⁴;
R² represents halogen, or cyano; or C₁₋₄ alkyl, C₃₋₇ cycloalkyl or C₁₋₄ alkoxy, any of which groups may be optionally substituted by one or more substituents;
R³ represents hydrogen, halogen or cyano; or C₁₋₄ alkyl, which group may be optionally substituted by one or more substituents; or
R² and R³ together with the group to which they are attached form a cycloalkyl, heterocyclyl, aryl or heteroaryl, which group is optionally substituted with one or more substituents;
R¹⁸ represents hydrogen or halogen; or C₁₋₄ alkyl which may be optionally substituted by one or more substituents; and
R¹⁹, R²⁰, R²¹, R²², R²³ and R²⁴ represent independently hydrogen or halogen; or C₁₋₄ alkyl which may be optionally substituted by one or more substituents.

2. A compound according to claim 1, wherein Q represents an optionally substituted ring selected from the groups represented by
Q³, Q⁴, Q⁵ and Q⁸: 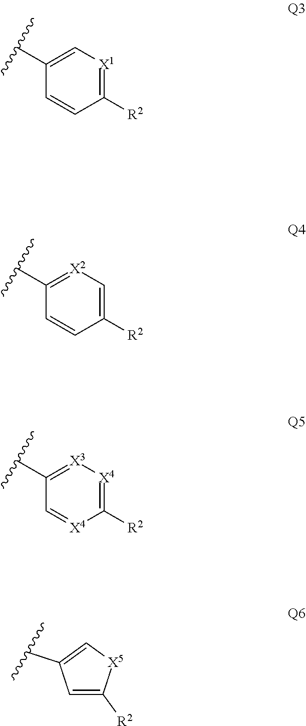
Wherein
X¹ represents N or C—R³;
X² represents N or C—R¹⁹;
X³ represents C—R¹⁹;
one of the two X⁴ represents C—R²⁰ and the other X⁴ represents C—H;
X⁵ represents S; and
R³, R¹⁹ and R²⁰ are as defined in claim 1.

3. A compound of formula (I) according to claim 1, or a pharmaceutically acceptable salt thereof, represented by formula (IA), 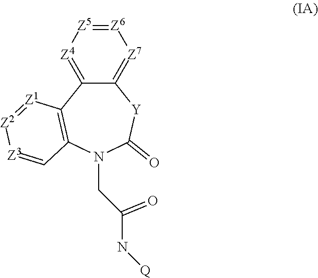
Wherein Q, Y, Z¹, Z², Z³, Z⁴, Z⁵, Z⁶ and Z⁷ are as defined in claim 1.

4. A compound of formula (I) according to claim 1, or a pharmaceutically acceptable salt thereof, represented by formula (IB), 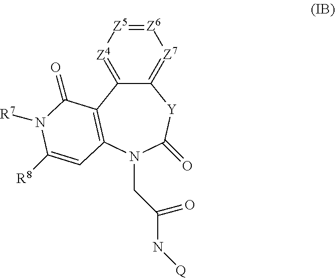
Wherein Q, Y, Z⁴, Z⁵, Z⁶, Z⁷, R⁷ and R⁸ are as defined in claim 1.

5. A compound of formula (I) according to claim 1, or a pharmaceutically acceptable salt thereof, represented by formula (IC), 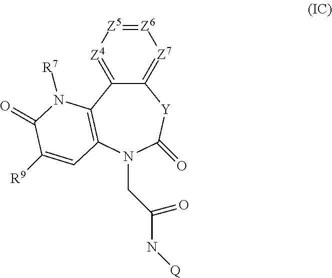
Wherein Q, Y, Z⁴, Z⁵, Z⁶, Z⁷, R⁷ and R⁹ are as defined in claim 1.

6. A compound of formula (I) according to claim 1, or a pharmaceutically acceptable salt thereof, represented by formula (ID), 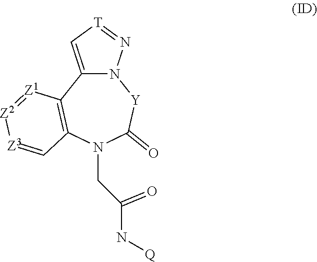
Wherein Q, Y, Z¹, Z², Z³, and T are as defined in claim 1.

7. A compound of formula (I) according to claim 1, or a pharmaceutically acceptable salt thereof, represented by formula (IE), 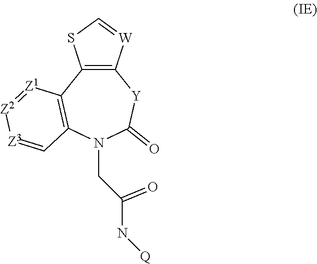
Wherein Q, Y, Zi, Z², Z³ and W are as defined in claim 1.

8. A compound of formula (I) according to claim 1 wherein
Y represents N—R¹ or CR^{1a}R^{1b};
R^{a} represents methyl;
R^{1a} represents hydrogen, deuterium, fluoro, hydroxyl, methyl, deuterated methyl (—CD₃), hydroxymethyl, methoxymethyl, or methoxy;
R^{1b} represents hydrogen or methyl;
A, together with the points of attachment to the remainder of the molecule, V³ and V⁴, represents an optionally substituted aryl or heteroaryl selected from the groups represented by A¹, A², A³, A⁴, A⁵, A⁶ and A⁷;
Z¹ represents N or C—R⁴, Z² represents N or C—R⁵′, and Z³ represents N or C—R⁶, wherein one or none of Z¹, Z² and Z³ represents N;
R^{e}represents hydrogen or fluoro;
R⁴ represents hydrogen, chloro, fluoro, cyano, hydroxyl, amino, methyl, ethyl, difluoromethyl, hydroxymethyl, cyclopropyl, methoxy, ethoxy, or (methoxy)ethoxy;
R⁵ represents hydrogen, fluoro, cyano, methyl, or methoxy;
R⁶ represents hydrogen, chloro, fluoro, cyano, methoxy, methylamino, methyl, (hydroxy)methyl, (methylcarboxy)methyl, (oxo)(methyl)piperazinyl, (difluoro)azetidinyl, azaspirohexanyl, azabicycloheptanyl, (difluoro)azaspirohexanyl, morpholino, (hydroxymethyl)azetidinyl, (fluoroazetidinyl), (methyl)(hydroxyl)azetidinyl, (fluoro)pyrrolidinyl, (difluoro)pyrrolidinyl, (hydroxyl)pyrrolidinyl, (difluoro)azaspiroheptanyl, oxa-azaspirooctanyl, oxa-azasprioheptanyl, (fluoro)(methyl)azetidinyl, (difluoro)azabicyclohexanyl, (diflouro)azabicycloheptanyl, (methyl)azetidinyl, or (azetidinyl)oxy;
R⁷ represents hydrogen, methyl, ethyl, or cyclopropyl;
R⁸ represents hydrogen, methyl or methoxy;
R⁹ represents hydrogen, fluoro, methyl or cyclopropyl;
R¹⁰ represents hydrogen or fluoro; R¹¹ represents —NR^{c}—(CO)—R^{b}; R^{b} represents methyl; R^{c} represents methyl;
B, together with the points of attachment to the remainder of the molecule, V¹ and V², represents an optionally substituted aryl or heteroaryl selected from the groups represented by B¹, B², B³, B4, B5, B⁶, B⁷ and B;
V¹ represents C for B¹, B², B³, B⁴, B⁵, B⁶ and B⁷ and represents N for B;
V² represents C for B¹, B², B⁴, B⁶, B⁷, and B⁸ and represents N for B³ and B⁵;
W, U¹ and U² represent independently N or C—H;
Z⁴ represents N or C—R¹³, Z⁵ represents N or C—R¹⁴, Z⁶ represents N or C—R¹⁵, and Z⁷ represents N or C—R¹⁶, wherein none, one or two of Z⁴, Z⁵, Z⁶ and Z⁷ represents N;
T represents N or C—R¹⁷; R¹² represents hydrogen;
R¹³ represents hydrogen, fluoro or chloro;
R¹⁴ represents hydrogen, fluoro, chloro or methoxy;
R¹⁵ represents hydrogen, fluoro, chloro or methyl;
R¹⁶ represents hydrogen, fluoro, chloro, cyano or (hydroxy)methyl;
R¹⁷ represents hydrogen, methyl or fluoro; R^{17′} represents hydrogen or methyl;
Q represents an optionally substituted ring selected from the groups represented by Q³, Q⁴, Q⁵ and Q⁶;
X¹ represents N or C—R³; X² represents N or C—R¹⁹; X³ represents C—R¹⁹;
one of the two X⁴ represents C—R²⁰ and the other X⁴ represents C—H;
X⁵ represents S;
R² represents chloro, bromo, iodo, fluoro, cyano, methyl, difluoromethyl, trifluoromethyl, difluoroethyl, cyclopropyl, difluorocyclopropyl, methoxy, or difluoromethoxy;
R³ represents hydrogen, chloro, fluoro or methyl;
or R² and R³ together with the group to which they are attached form (methyl)indazolyl, (difluoromethyl)indazolyl, (methyl)triazolyl, (difluoro)indanyl, (difluoro)benzodioxolyl, (fluoro)isoquinolyl and (chloro)isoquinolyl;
R¹⁹ represents hydrogen or fluoro; and
R²⁰ represents hydrogen or fluoro.

9. A compound according to claim 1 wherein Q represents Q³.

10. A compound according to claim 1, wherein Y represents CR^{1a}R^{1b}.

11. A compound according to claim 1, wherein Z¹ represents C—R⁴, Z² represents N, and Z³ represents C—R⁶.

12. A compound according claim 1, wherein Z⁴ represents C—R¹³, Z⁵ represents C—R¹⁴, Z⁶ represents N and Z⁷ represents C—R¹⁶.

13. A compound of formula (I) according to claim 1 represented by compound of formula (IG), 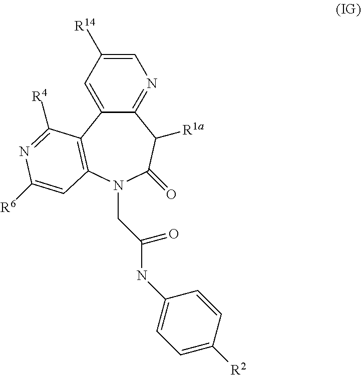
Wherein
R^{1a} is methyl or hydroxyl;
R² is difluoromethyl or difluoroethyl;
R⁴ is hydrogen, chloro or fluoro;
R⁶ is methyl; and
R¹⁴ is hydrogen or fluoro.

14. A compound as claimed in claim 1 which is
N-(4-chloro-3-fluoro-phenyl)-2-(9,10-dimethyl-6-oxo-5-H-pyrido[3,2-d][1]benzazepin-7-yl)acetamide;
N-(4-chloro-3-fluoro-phenyl)-2-(9,10-dimethyl-6-oxo-5H-pyrido[4,3-d][1]benzazepin-7-yl)acetamide;
N-(4-chloro-3-fluoro-phenyl)-2-(9,10-dimethyl-6-oxo-5-H-pyrido[2,3-d][1]benzazepin-7-yl)acetamide;
N-(4-chloro-3-fluoro-phenyl)-2-(3,9,10-trimethyl-6-oxo-5-H-pyrido[2,3-d][1]benzazepin-7-yl)acetamide;
2-(8,9-dimethyl-5-oxo-4H-pyrazolo[5,1-d][1,5]benzodiazepin-6-yl)-N-[4-(trifluoromethyl)phenyl]acetamide;
2-(5-methyl-9-oxo-4,8,14-triazatricyclo[9.4.0.02,7]pentadeca-1(11),2(7),3,5,12,14-hexaen-8-yl)-N-[4-(trifluoromethyl)phenyl]acetamide;
2-(13-chloro-9-oxo-3,10,14-triazatricyclo[9.4.0.02,7]pentadeca-1(11),2(7),3,5,12,14-hexaen-10-yl)-N-[4-(trifluoromethyl)phenyl]acetamide;
2-(8,9-dimethyl-5-oxo-4H-imidazo[1,2-a][1,5]benzodiazepin-6-yl)-N-[4-(trifluoromethyl)phenyl]acetamide;
2-(9,10-dimethyl-6-oxo-5H-pyrimido[5,4-d][1]benzazepin-7-yl)-N-[4-(trifluoromethyl)phenyl]acetamide;
2-(2-chloro-9,10-dimethyl-6-oxo-5H-pyrido[3,2-d][1]benzazepin-7-yl)-N-[4-(trifluoromethyl)phenyl]acetamide;
2-(5-chloro-9-oxo-4,8,14-triazatricyclo[9.4.0.02,7]pentadeca-1(11),2(7),3,5,12,14-hexaen-8-yl)-N-[4-(trifluoromethyl)phenyl]acetamide;
2-(13-methyl-9-oxo-3,10,14-triazatricyclo[9.4.0.02,7]pentadeca-1(11),2(7),3,5,12,14-hexaen-10-yl)-N-[4-(trifluoromethyl)phenyl]acetamide;
2-(3-chloro-6-oxo-7H-pyrido[4,3-d][3]benzazepin-5-yl)-N-[4-(trifluoromethyl)phenyl]acetamide;
2-(3-methyl-6-oxo-7H-pyrido[4,3-d][3]benzazepin-5-yl)-N-[4-(trifluoromethyl)phenyl]acetamide;
2-(2,3-dimethyl-6-oxo-7H-pyrido[3,2-d][3]benzazepin-5-yl)-N-[4-(trifluoromethyl)phenyl]acetamide;
2-(3-fluoro-6-oxo-7H-pyrido[3,2-d][3]benzazepin-5-yl)-N-[4-(trifluoromethyl)phenyl]acetamide;
2-(6-oxo-7H-pyrido[4,3-d][3]benzazepin-5-yl)-N-[4-(trifluoromethyl)phenyl]acetamide;
N-(3-chloro-4-methyl-phenyl)-2-(13-chloro-9-oxo-3,10,14-triazatricyclo[9.4.0.02,7]pentadeca-1(11),2(7),3,5,12,14-hexaen-10-yl)acetamide;
N-(4-bromo-3-chloro-phenyl)-2-(13-chloro-9-oxo-3,10,14-triazatricyclo[9.4.0.02,7]pentadeca-1(11),2(7),3,5,12,14-hexaen-10-yl)acetamide;
N-(4-chloro-3-fluoro-phenyl)-2-(13-chloro-9-oxo-3,10,14-triazatricyclo[9.4.0.02,7]pentadeca-1(11),2(7),3,5,12,14-hexaen-10-yl)acetamide;
N-[4-(trifluoromethyl)phenyl]-2-(3,9,10-trimethyl-6-oxo-5H-pyrido[4,3-d][1]benzazepin-7-yl)acetamide;
2-(9,10-dimethyl-6-oxo-5H-pyrido[3,2-d][1]benzazepin-7-yl)-N-[4-(trifluoromethyl)phenyl]acetamide;
2-(9,10-dimethyl-6-oxo-5H-pyrido[2,3-d][1]benzazepin-7-yl)-N-[4-(trifluoromethyl)phenyl]acetamide;
2-(9,10-dimethyl-6-oxo-5H-pyrido[4,3-d][1]benzazepin-7-yl)-N-[4-(trifluoromethyl)phenyl]acetamide;
2-(5-chloro-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(12-chloro-8-oxo-5-thia-9,13-diazatricyclo[8.4.0.02,6]tetradeca-1(10),2(6),3,11,13-pentaen-9-yl)-N-[4-(trifluoromethyl)phenyl]acetamide;
2-(1,3-dimethyl-6-oxo-7H-pyrido[4,3-d][3]benzazepin-5-yl)-N-[4-(trifluoromethyl)phenyl]acetamide;
2-(1-methyl-6-oxo-7H-pyrido[4,3-d][3]benzazepin-5-yl)-N-[4-(trifluoromethyl)phenyl]acetamide;
2-(2-methyl-6-oxo-7H-pyrido[3,4-d][3]benzazepin-5-yl)-N-[4-(trifluoromethyl)phenyl]acetamide;
2-(10-chloro-3-methyl-6-oxo-7H-pyrido[4,3-d][3]benzazepin-5-yl)-N-[4-(trifluoromethyl)phenyl]acetamide;
2-(8-chloro-3-methyl-6-oxo-7H-pyrido[4,3-d][3]benzazepin-5-yl)-N-[4-(trifluoromethyl)phenyl]acetamide;
2-(11-chloro-3-methyl-6-oxo-7H-pyrido[4,3-d][3]benzazepin-5-yl)-N-[4-(trifluoromethyl)phenyl]acetamide;
2-(10-fluoro-3-methyl-6-oxo-7H-pyrido[4,3-d][3]benzazepin-5-yl)-N-[4-(trifluoromethyl)phenyl]acetamide;
2-(9-fluoro-3-methyl-6-oxo-7H-pyrido[4,3-d][3]benzazepin-5-yl)-N-[4-(trifluoromethyl)phenyl]acetamide;
2-(8-fluoro-3-methyl-6-oxo-7H-pyrido[4,3-d][3]benzazepin-5-yl)-N-[4-(trifluoromethyl)phenyl]acetamide;
2-(5-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(12-chloro-8-oxo-5,6,9,13-tetrazatricyclo[8.4.0.02,6]tetradeca-1(10),2,4,11,13-pentaen-9-yl)-N-[4-(trifluoromethyl)phenyl]acetamide;
2-(15-fluoro-13-methyl-9-oxo-3,10,14-triazatricyclo[9.4.0.02,7]pentadeca-1(11),2(7),3,5,12,14-hexaen-10-yl)-N-[4-(trifluoromethyl)phenyl]acetamide;
2-(1-fluoro-3-methyl-6-oxo-7H-pyrido[4,3-d][3]benzazepin-5-yl)-N-[4-(trifluoromethyl)phenyl]acetamide;
2-(3-fluoro-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
N-(4-chloro-3-fluoro-phenyl)-2-(15-fluoro-13-methyl-9-oxo-3,10,14-triazatricyclo[9.4.0.0^{2,7}]pentadeca-1(11),2(7),3,5,12,14-hexaen-10-yl)acetamide;
2-(14-fluoro-12-methyl-8-oxo-3-thia-9,13-diazatricyclo[8.4.0.0^{2,6}]tetradeca-1(10),2(6),4,11,13-pentaen-9-yl)-N-[4-(trifluoromethyl)phenyl]acetamide;
2-(3,5-difluoro-9-oxo-4,8,12-triazatricyclo[9.4.0.0^{2,7}]pentadeca-1(11),2,4,6,12,14-hexaen-8-yl)-N-[4-(trifluoromethyl)phenyl]acetamide;
2-(9-chloro-3-methyl-6-oxo-7H-pyrido[4,3-d][3]benzazepin-5-yl)-N-[4-(trifluoromethyl)phenyl]acetamide;
2-(1-methyl-6-oxo-7H-pyrido[3,4-d][3]benzazepin-5-yl)-N-[4-(trifluoromethyl)phenyl]acetamide;
2-[3-(4-methyl-3-oxo-piperazin-1-yl)-6-oxo-7H-pyrido[4,3-d][3]benzazepin-5-yl]-N-[4-(trifluoromethyl)phenyl]acetamide;
2-[3-(3,3-difluoroazetidin-1-yl)-6-oxo-7H-pyrido[4,3-d][3]benzazepin-5-yl]-N-[4-(trifluoromethyl)phenyl]acetamide;
2-(12-methyl-8-oxo-5,6,9,13-tetrazatricyclo[8.4.0.02,6]tetradeca-1(10),2,4,11,13-pentaen-9-yl)-N-[4-(trifluoromethyl)phenyl]acetamide;
2-(1-chloro-3-methyl-6-oxo-7H-pyrido[4,3-d][3]benzazepin-5-yl)-N-[4-(trifluoromethyl)phenyl]acetamide;
N-(4-chloro-3-fluoro-phenyl)-2-(1-chloro-3-methyl-6-oxo-7H-pyrido[4,3-d][3]benzazepin-5-yl)acetamide;
2-(1-chloro-9-fluoro-6-oxo-7H-pyrido[4,3-d][3]benzazepin-5-yl)-N-[4-(trifluoromethyl)phenyl]acetamide;
2-(9,11-dichloro-10-fluoro-6-oxo-5H-pyrido[2,3-d][1]benzazepin-7-yl)-N-[4-(trifluoromethyl)phenyl]acetamide;
2-(3-chloro-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
2-(10-cyano-11-fluoro-6-oxo-5H-pyrido[2,3-d][1]benzazepin-7-yl)-N-[4-(trifluoromethyl)phenyl]acetamide;
2-(1-hydroxy-3-methyl-6-oxo-7H-pyrido[4,3-d][3]benzazepin-5-yl)-N-[4-(trifluoromethyl)phenyl]acetamide;
2-[1-(difluoromethyl)-3-methyl-6-oxo-7H-pyrido[4,3-d][3]benzazepin-5-yl]-N-[4-(trifluoromethyl)phenyl]acetamide;
2-(3,5-dimethyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
N-(4-cyclopropylphenyl)-2-(3,5-dimethyl-9-oxo-4.8,12-triazatricyclo[9.4.0.02.7]pentadeca-1(11),2(7),3,5,12,14-hexaen-8-yl)acetamide;
[6-oxo-5-[2-oxo-2-[4-(trifluoromethyl)anilino]ethyl]-7H-pyrido[4,3-d][3]benzazepin-3-yl]methyl acetate 2-[3-(hydroxymethyl)-6-oxo-7H-pyrido[4,3-d][3]benzazepin-5-yl]-N-[4-(trifluoromethyl)phenyl]acetamide;
2-(1-cyano-3-methyl-6-oxo-7H-pyrido[4,3-d][3]benzazepin-5-yl)-N-[4-(trifluoromethyl)phenyl]acetamide;
2-(9-chloro-1,3-dimethyl-6-oxo-7H-pyrido[4,3-d][3]benzazepin-5-yl)-N-[4-(trifluoromethyl)phenyl]acetamide;
2-(10-fluoro-1,3-dimethyl-6-oxo-7H-pyrido[4,3-d][3]benzazepin-5-yl)-N-[4-(trifluoromethyl)phenyl]acetamide;
2-(8-fluoro-1,3-dimethyl-6-oxo-7H-pyrido[4,3-d][3]benzazepin-5-yl)-N-[4-(trifluoromethyl)phenyl]acetamide;
2-(9-fluoro-1,3-dimethyl-6-oxo-7H-pyrido[4,3-d][3]benzazepin-5-yl)-N-[4-(trifluoromethyl)phenyl]acetamide;
2-[1-(hydroxymethyl)-3-methyl-6-oxo-7H-pyrido[4,3-d][3]benzazepin-5-yl]-N-[4-(trifluoromethyl)phenyl]acetamide;
2-(4-methoxy-3-methyl-9-oxo-5,8,12-triazatricyclo[9.4.0.0^{2,7}]pentadeca-1(11),2(7),3,5,12,14-hexaen-8-yl)-N-[4-(trifluoromethyl)phenyl]acetamide;
2-(11-cyano-10-fluoro-6-oxo-5H-pyrido[2,3-d][1]benzazepin-7-yl)-N-[4-(trifluoromethyl)phenyl]acetamide;
2-(9-cyano-10-fluoro-6-oxo-5H-pyrido[2,3-d][1]benzazepin-7-yl)-N-[4-(trifluoromethyl)phenyl]acetamide;
N-[4-(trifluoromethyl)phenyl]-2-(3,5,13-trimethyl-9-oxo-4,8,12-triazatricyclo[9.4.0.02,7]pentadeca-1(11),2(7),3,5,12,14-hexaen-8-yl)acetamide;
2-(5,13-dimethyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(1-ethyl-3-methyl-6-oxo-7H-pyrido[4,3-d][3]benzazepin-5-yl)-N-[4-(trifluoromethyl)phenyl]acetamide;
2-(1-cyclopropyl-3-methyl-6-oxo-7H-pyrido[4,3-d][3]benzazepin-5-yl)-N-[4-(trifluoromethyl)phenyl]acetamide;
2-(14-methoxy-12-methyl-8-oxo-4,5,6,9,13-pentazatricyclo[8.4.0.02,6]tetradeca-1(10),2,4,11,13-pentaen-9-yl)-N-[4-(trifluoromethyl)phenyl]acetamide;
2-(3-methoxy-5-methyl-9-oxo-4,8,12-triazatricyclo[9.4.0.02,7]pentadeca-1(11),2,4,6,12,14-hexaen-8-yl)-N-[4-(trifluoromethyl)phenyl]acetamide;
2-(3-hydroxy-5-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
N-(4-cyanophenyl)-2-(3-methoxy-5-methyl-9-oxo-4,8,12-triazatricyclo[9.4.0.0^{2,7}]pentadeca-1(11),2,4,6,12,14-hexaen-8-yl)acetamide;
2-(3-fluoro-9-oxo-5,8,12-triazatricyclo[9.4.0.0^{2,7}]pentadeca-1(11),2(7),3,5,12,14-hexaen-8-yl)-N-[4-(trifluoromethyl)phenyl]acetamide;
2-(3-methoxy-5-methyl-9-oxo-4,8,12-triazatricyclo[9.4.0.0^{2,7}]pentadeca-1(11),2,4,6,12,14-hexaen-8-yl)-N-[6-(trifluoromethyl)-3-pyridyl]acetamide;
N-(4-cyclopropylphenyl)-2-(3-methoxy-5-methyl-9-oxo-4,8,12-triazatricyclo[9.4.0.0^{2,7}]pentadeca-1(11),2(7),3,5,12,14-hexaen-8-yl)acetamide;
2-(3-methoxy-5,10-dimethyl-9-oxo-4,8,12-triazatricyclo[9.4.0.0^{2,7}]pentadeca-1(11),2,4,6,12,14-hexaen-8-yl)-N-[4-(trifluoromethyl)phenyl]acetamide;
2-(3-ethoxy-5-methyl-9-oxo-4,8,12-triazatricyclo[9.4.0.0^{2,7}]pentadeca-1(15),2(7),3,5,11,13-hexaen-8-yl)-N-[4-(trifluoromethyl)phenyl]acetamide;
N-[4-(difluoromethyl)phenyl]-2-(3-methoxy-5-methyl-9-oxo-4,8,12-triazatricyclo[9.4.0.0^{2,7}]pentadeca-1(11),2,4,6,12,14-hexaen-8-yl)acetamide;
2-(3-fluoro-5,10-dimethyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(9-chloro-1-fluoro-3-methyl-6-oxo-7H-pyrido[4,3-d][3]benzazepin-5-yl)-N-[4-(trifluoromethyl)phenyl]acetamide;
2-(1,9-difluoro-3-methyl-6-oxo-7H-pyrido[4,3-d][3]benzazepin-5-yl)-N-[4-(trifluoromethyl)phenyl]acetamide;
2-(1,11-difluoro-3-methyl-6-oxo-7H-pyrido[4,3-d][3]benzazepin-5-yl)-N-[4-(trifluoromethyl)phenyl]acetamide;
2-(8-cyano-1-fluoro-3-methyl-6-oxo-7H-pyrido[4,3-d][3]benzazepin-5-yl)-N-[4-(trifluoromethyl)phenyl]acetamide;
2-(3,10-difluoro-5,10-dimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
N-(4-chloro-3-fluoro-phenyl)-2-(3-fluoro-5,10-dimethyl-9-oxo-4,8,12-triazatricyclo[9.4.0.0^{2,7}]pentadeca-1(11),2(7),3,5,12,14-hexaen-8-yl)acetamide;
2-(5,10-dimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
2-(3-fluoro-5-methyl-9-oxo-10-(trideuteriomethyl)-4,8,12-triazatricyclo[9.4.0.0^{2,7}]pentadeca-1(15),2(7),3,5,11,13-hexaen-8-yl)-N-[4-(trifluoromethyl)phenyl]acetamide;
2-(3-fluoro-5,10,13-trimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
2-(3-fluoro-10-hydroxy-5-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(1-fluoro-7-hydroxy-3-methyl-6-oxo-7H-pyrido[4,3-d][3]benzazepin-5-yl)-N-[4-(trifluoromethyl)phenyl]acetamide;
2-(10-hydroxy-3-methoxy-5-methyl-9-oxo-4,8,12-triazatricyclo[9.4.0.02,7]pentadeca-1(11),2,4,6,12,14-hexaen-8-yl)-N-[4-(trifluoromethyl)phenyl]acetamide;
2-(3-chloro-5-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(15-methoxy-13-methyl-9-oxo-4,6,10,14-tetrazatricyclo[9.4.0.02,7]pentadeca-1(15),2(7),3,5,11,13-hexaen-10-yl)-N-[4-(trifluoromethyl)phenyl]acetamide;
2-(14-methoxy-12-methyl-8-oxo-5,6,9,13-tetrazatricyclo[8.4.0.02,6]tetradeca-1(14),2,4,10,12-pentaen-9-yl)-N-[4-(trifluoromethyl)phenyl]acetamide;
2-(14-chloro-12-methyl-8-oxo-5,6,9,13-tetrazatricyclo[8.4.0.02,6]tetradeca-1(14),2,4,10,12-pentaen-9-yl)-N-[4-(trifluoromethyl)phenyl]acetamide;
2-(3,10-difluoro-5-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(3-fluoro-5-methyl-9,12-dioxo-4,8,11-triazatricyclo[9.4.0.02,7]pentadeca-1(15),2(7),3,5,13-pentaen-8-yl)-N-[4-(trifluoromethyl)phenyl]acetamide;
2-(3-amino-5-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(14-methoxy-4,12-dimethyl-8-oxo-5,6,9,13-tetrazatricyclo[8.4.0.02,6]tetradeca-1(14),2,4,10,12-pentaen-9-yl)-N-[4-(trifluoromethyl)phenyl]acetamide;
2-(14-chloro-4,12-dimethyl-8-oxo-5,6,9,13-tetrazatricyclo[8.4.0.02,6]tetradeca-1(14),2,4,10,12-pentaen-9-yl)-N-[4-(trifluoromethyl)phenyl]acetamide;
2-(14-fluoro-4,12-dimethyl-8-oxo-5,6,9,13-tetrazatricyclo[8.4.0.02,6]tetradeca-1(10),2,4,11,13-pentaen-9-yl)-N-[4-(trifluoromethyl)phenyl]acetamide;
2-(1-chloro-7-methoxy-3-methyl-6-oxo-7H-pyrido[3,4-a][3]benzazepin-5-yl)-N-[4-(trifluoromethyl)phenyl]acetamide;
2-(3,5-dimethoxy-9-oxo-4,8,12-triazatricyclo[9.4.0.02,7]pentadeca-1(11),2,4,6,12,14-hexaen-8-yl)-N-[4-(trifluoromethyl)phenyl]acetamide;
2-(4-methoxy-5,10-dimethyl-9-oxo-3,8,12-triazatricyclo[9.4.0.02,7]pentadeca-1(11),2,4,6,12,14-hexaen-8-yl)-N-[4-(trifluoromethyl)phenyl]acetamide;
2-(3-fluoro-5-methyl-9-oxo-4,8,12,13-tetrazatricyclo[9.4.0.02,7]pentadeca-1(11),2,4,6,12,14-hexaen-8-yl)-N-[4-(trifluoromethyl)phenyl]acetamide;
2-(14-fluoro-7,12-dimethyl-8-oxo-5,6,9,13-tetrazatricyclo[8.4.0.02,6]tetradeca-1(10),2,4,11,13-pentaen-9-yl)-N-[4-(trifluoromethyl)phenyl]acetamide;
2-(15-fluoro-8,13-dimethyl-9-oxo-4,6,10,14-tetrazatricyclo[9.4.0.02,7]pentadeca-1(15),2(7),3,5,11,13-hexaen-10-yl)-N-[4-(trifluoromethyl)phenyl]acetamide;
2-(5-fluoro-3-methoxy-9-oxo-4,8,12-triazatricyclo[9.4.0.02,7]pentadeca-1(11),2,4,6,12,14-hexaen-8-yl)-N-[4-(trifluoromethyl)phenyl]acetamide;
2-(3-chloro-5,10-dimethyl-9-oxo-4,8,12-triazatricyclo[9.4.0.02,7]pentadeca-1(11),2,4,6,12,14-hexaen-8-yl)-N-[4-(trifluoromethyl)phenyl]acetamide;
2-(3-chloro-5,10-dimethyl-9-oxo-4,8,12-triazatricyclo[9.4.0.02,7]pentadeca-1(11),2,4,6,12,14-hexaen-8-yl)-N-(4-cyanophenyl)acetamide;
Enantiomer (10R) of 2-[3-chloro-5,10-dimethyl-9-oxo-4,8,12-triazatricyclo[9.4.0.02,7]pentadeca-1(11),2(7),3,5,12,14-hexaen-8-yl]-N-[4-(difluoromethyl)phenyl]acetamide;
Enantiomer (10S) of 2-[3-chloro-5,10-dimethyl-9-oxo-4,8,12-triazatricyclo[9.4.0.02,7]pentadeca-1(11),2(7),3,5,12,14-hexaen-8-yl]-N-[4-(difluoromethyl)phenyl]acetamide;
Enantiomer (10R) of 2-[3-chloro-5,10-dimethyl-9-oxo-4,8,12-triazatricyclo[9.4.0.02,7]pentadeca-1(15),2(7),3,5,11,13-hexaen-8-yl]-N-(4-chloro-3-fluoro-phenyl)acetamide;
Enantiomer (10S) of 2-[3-chloro-5,10-dimethyl-9-oxo-4,8,12-triazatricyclo[9.4.0.02,7]pentadeca-1(15),2(7),3,5,11,13-hexaen-8-yl]-N-(4-chloro-3-fluoro-phenyl)acetamide;
2-[2-[acetyl(methyl)amino]-5-oxo-6H-thiazolo[4,5-d][3]benzazepin-4-yl]-N-[4-(trifluoromethyl)phenyl]acetamide;
N-[4-(trifluoromethyl)phenyl]-2-(3,5,10-trimethyl-4,9-dioxo-3,8,12-triazatricyclo[9.4.0.02,7]pentadeca-1(11),2(7),5,12,14-pentaen-8-yl)acetamide;
N-(3-fluoro-4-methyl-phenyl)-2-(1,3,7-trimethyl-2,6-dioxo-7H-pyrido[3,2-d][3]benzazepin-5-yl)acetamide;
Enantiomer (7S) of N-[4-(trifluoromethyl)phenyl]-2-[1,3,7-trimethyl-2,6-dioxo-7H-pyrido[3,2-d][3]benzazepin-5-yl]acetamide;
Enantiomer (7R) of N-[4-(trifluoromethyl)phenyl]-2-[1,3,7-trimethyl-2,6-dioxo-7H-pyrido[3,2-d][3]benzazepin-5-yl]acetamide;
2-[1-chloro-7-(methoxymethyl)-3-methyl-6-oxo-7H-pyrido[3,4-a][3]benzazepin-5-yl]-N-[4-(trifluoromethyl)phenyl]acetamide;
2-(14-fluoro-12-methyl-8-oxo-4,5,9,13-tetrazatricyclo[8.4.0.02,6]tetradeca-1(10),2,5,11,13-pentaen-9-yl)-N-[4-(trifluoromethyl)phenyl]acetamide;
2-(3-fluoro-5-methoxy-9-oxo-4,8,12-triazatricyclo[9.4.0.02,7]pentadeca-1(11),2,4,6,12,14-hexaen-8-yl)-N-[4-(trifluoromethyl)phenyl]acetamide;
2-(4-ethyl-5-methyl-3,9-dioxo-4,8,12-triazatricyclo[9.4.0.02,7]pentadeca-1(15),2(7),5,11,13-pentaen-8-yl)-N-[4-(trifluoromethyl)phenyl]acetamide;
2-(3-chloro-5,10-dimethyl-9-oxo-4,8,10,12-tetrazatricyclo[9.4.0.02,7]pentadeca-1(11),2,4,6,12,14-hexaen-8-yl)-N-[4-(trifluoromethyl)phenyl]acetamide;
2-(1-cyano-10-fluoro-3-methyl-6-oxo-7H-pyrido[4,3-d][3]benzazepin-5-yl)-N-[4-(trifluoromethyl)phenyl]acetamide;
2-(3-fluoro-5,10-dimethyl-9-oxo-4,8,12-triazatricyclo[9.4.0.02,7]pentadeca-1(11),2(7),3,5,12,14-hexaen-8-yl)-N-(3-fluoro-4-methyl-phenyl)acetamide;
2-(3-fluoro-5,10-dimethyl-9-oxo-4,8,12-triazatricyclo[9.4.0.0^{2,7}]pentadeca-1(11),2(7),3,5,12,14-hexaen-8-yl)-N-(1-methylindazol-5-yl)acetamide;
N-(3,4-difluorophenyl)-2-(3-fluoro-5,10-dimethyl-9-oxo-4,8,12-triazatricyclo[9.4.0.0^{2,7}]pentadeca-1(11),2(7),3,5,12,14-hexaen-8-yl)acetamide;
2-(3-fluoro-5,10-dimethyl-9-oxo-4,8,12-triazatricyclo[9.4.0.0^{2,7}]pentadeca-1(11),2(7),3,5,12,14-hexaen-8-yl)-N-(3-fluoro-4-methoxy-phenyl)acetamide;
N-[4-(difluoromethyl)phenyl]-2-(3-fluoro-5,10-dimethyl-9-oxo-4,8,12-triazatricyclo[9.4.0.0^{2,7}]pentadeca-1(11),2(7),3,5,12,14-hexaen-8-yl)acetamide;
N-(4-cyano-3-fluoro-phenyl)-2-(3-fluoro-5,10-dimethyl-9-oxo-4,8,12-triazatricyclo[9.4.0.0^{2,7}]pentadeca-1(11),2(7),3,5,12,14-hexaen-8-yl)acetamide;
N-[4-(difluoromethyl)phenyl]-2-(1-fluoro-3-methyl-6-oxo-7H-pyrido[4,3-d][3]benzazepin-5-yl)acetamide;
N-(4-cyclopropyl-3-fluoro-phenyl)-2-(3-fluoro-5,10-dimethyl-9-oxo-4,8,12-triazatricyclo[9.4.0.0^{2,7}]pentadeca-1(11),2(7),3,5,12,14-hexaen-8-yl)acetamide;
N-[4-(difluoromethoxy)-3-fluoro-phenyl]-2-(3-fluoro-5,10-dimethyl-9-oxo-4,8,12-triazatricyclo[9.4.0.0^{2,7}]pentadeca-1(11),2(7),3,5,12,14-hexaen-8-yl)acetamide;
2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]-N-[3-methyl-4-(trifluoromethyl)phenyl]acetamide;
2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]-N-(4-iodophenyl)acetamide;
N-[4-(difluoromethoxy)phenyl]-2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetamide;
N-(3,4-difluorophenyl)-2-[(10R)-3-fluoro-5,10-dimethyl-9-oxo-4,8,12-triazatricyclo[9.4.0.0²,7pentadeca-1(11),2(7),3,5,12,14-hexaen-8-yl]acetamide;
2-[(10R)-3-fluoro-5,10-dimethyl-9-oxo-4,8,12-triazatricyclo[9.4.0.0²,7pentadeca-1(11),2(7),3,5,12,14-hexaen-8-yl]-N-(1-methylindazol-5-yl)acetamide;
N-(4-cyclopropylphenyl)-2-[(10R)-3-fluoro-5,10-dimethyl-9-oxo-4,8,12-triazatricyclo[9.4.0.0²,7pentadeca-1(11),2(7),3,5,12,14-hexaen-8-yl]acetamide;
2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl)-N-[3-fluoro-4-(trifluoromethyl)phenyl]acetamide;
N-(4-cyclopropyl-3-fluoro-phenyl)-2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetamide;
N-(4-bromophenyl)-2-[(10R)-3-fluoro-5,10-dimethyl-9-oxo-4,8,12-triazatricyclo[9.4.0.0²,7pentadeca-1(11),2(7),3,5,12,14-hexaen-8-yl]acetamide;
Enantiomer (1S) of N-[4-(2,2-difluorocyclopropyl)phenyl]-2-[(10R)-3-fluoro-5,10-dimethyl-9-oxo-4,8,12-triazatricyclo[9.4.0.0²,71pentadeca-1(11),2(7),3,5,12,14-hexaen-8-yl]acetamide;
Enantiomer (1R) of N-[4-(2,2-difluorocyclopropyl)phenyl]-2-[(10R)-3-fluoro-5,10-dimethyl-9-oxo-4,8,12-triazatricyclo[9.4.0.0²,71pentadeca-1(11),2(7),3,5,12,14-hexaen-8-yl]acetamide;
2-[(10R)-3-fluoro-5,10-dimethyl-9-oxo-4,8,12-triazatricyclo[9.4.0.0²,7pentadeca-1(11),2(7),3,5,12,14-hexaen-8-yl]-N-(1-methylbenzotriazol-5-yl)acetamide;
N-[1-(difluoromethyl)indazol-5-yl]-2-[(10R)-3-fluoro-5,10-dimethyl-9-oxo-4,8,12-triazatricyclo[9.4.0.0²,71pentadeca-1(11),2(7),3,5,12,14-hexaen-8-yl]acetamide;
N-[4-(1,1-difluoroethyl)phenyl]-2-[(10R)-3-fluoro-5,10-dimethyl-9-oxo-4,8,12-triazatricyclo[9.4.0.0²,7pentadeca-1(11),2(7),3,5,12,14-hexaen-8-yl]acetamide;
N-(3,4-dichlorophenyl)-2-[(10R)-3-fluoro-5,10-dimethyl-9-oxo-4,8,12-triazatricyclo[9.4.0.0²,7pentadeca-1(11),2(7),3,5,12,14-hexaen-8-yl]acetamide;
N-[4-(difluoromethyl)phenyl]-2-[(10R)-3-fluoro-5,10-dimethyl-9-oxo-4,8,12-triazatricyclo[9.4.0.0²,7pentadeca-1(11),2(7),3,5,12,14-hexaen-8-yl]acetamide;
2-[3-fluoro-10-(hydroxymethyl)-5-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(3-cyano-5-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(3-cyano-5,10-dimethyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
Enantiomer (10R) of N-(4-chloro-3-fluoro-phenyl)-2-[3-cyano-5,10-dimethyl-9-oxo-4,8,12-triazatricyclo[9.4.0.02,7]pentadeca-1(11),2(7),3,5,12,14-hexaen-8-yl]acetamide;
Enantiomer (10S) of N-(4-chloro-3-fluoro-phenyl)-2-[3-cyano-5,10-dimethyl-9-oxo-4,8,12-triazatricyclo[9.4.0.02,7]pentadeca-1(11),2(7),3,5,12,14-hexaen-8-yl]acetamide;
2-(3,7-dimethyl-1,6-dioxo-2,7-dihydropyrido[4,3-d][3]benzazepin-5-yl)-N-[4-(trifluoromethyl)phenyl]acetamide;
2-(15-methoxy-8,13-dimethyl-9-oxo-4,6,10,14-tetrazatricyclo[9.4.0.02,7]pentadeca-1(11),2(7),3,5,12,14-hexaen-10-yl)-N-[4-(trifluoromethyl)phenyl]acetamide;
N-(4-bromo-3-fluoro-phenyl)-2-[(10R)-3-fluoro-5,10-dimethyl-9-oxo-4,8,12-triazatricyclo[9.4.0.02,7]pentadeca-1(11),2(7),3,5,12,14-hexaen-8-yl]acetamide;
2-(3-chloro-10-methoxy-5-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(3-chloro-12-fluoro-5-methyl-9-oxo-4,8,14-triazatricyclo[9.4.0.02,7]pentadeca-1(11),2(7),3,5,12,14-hexaen-8-yl)-N-[4-(trifluoromethyl)phenyl]acetamide;
Enantiomer (10R) of 2-(3-hydroxy-5,10-dimethyl-9-oxo-4,8,12-triazatricyclo[9.4.0.02,7pentadeca-1(11),2(7),3,5,12,14-hexaen-8-yl)-N-[4-(trifluoromethyl)phenyl]acetamide;
Enantiomer (10S) of 2-(3-hydroxy-5,10-dimethyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
Enantiomer (10R) of 2-[3-(2-methoxyethoxy)-5,10-dimethyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
Enantiomer (10S) of 2-[3-(2-methoxyethoxy)-5,10-dimethyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(15-chloro-8,13-dimethyl-9-oxo-4,6,10,14-tetrazatricyclo[9.4.0.02,7]pentadeca-1(11),2(7),3,5,12,14-hexaen-10-yl)-N-[4-(trifluoromethyl)phenyl]acetamide;
2-(1-chloro-10-methoxy-3,7-dimethyl-6-oxo-7H-pyrido[4,3-d][3]benzazepin-5-yl)-N-[4-(trifluoromethyl)phenyl]acetamide;
2-[1-fluoro-8-(hydroxymethyl)-3-methyl-6-oxo-7H-pyrido[4,3-d][3]benzazepin-5-yl]-N-[4-(trifluoromethyl)phenyl]acetamide;
N-(4-chloro-3-fluoro-phenyl)-2-(1,3,7-trimethyl-2,6-dioxo-pyrido[3,2-d][1,31benzodiazepin-5-yl)acetamide;
2-(5-cyano-3-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(3,5-dichloro-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-[3-chloro-5-(methylamino)-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-[3-chloro-5-(1-methylazetidin-3-yl)-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(3,12-difluoro-5,10-dimethyl-9-oxo-4,8,14-triazatricyclo[9.4.0.02,7]pentadeca-1(11),2(7),3,5,12,14-hexaen-8-yl)-N-[4-(trifluoromethyl)phenyl]acetamide;
2-(3-fluoro-5,10-dimethyl-9-oxo-4,8,12,13-tetrazatricyclo[9.4.0.02,7]pentadeca-1(11),2(7),3,5,12,14-hexaen-8-yl)-N-[4-(trifluoromethyl)phenyl]acetamide;
2-(3-chloro-5,10-dimethyl-9-oxo-4,8,12,13-tetrazatricyclo[9.4.0.02,7]pentadeca-1(11),2(7),3,5,12,14-hexaen-8-yl)-N-[4-(trifluoromethyl)phenyl]acetamide;
Enantiomer (7R) of N-(4-chloro-3-fluoro-phenyl)-2-(9-fluoro-2,3,7-trimethyl-1,6-dioxo-7H-pyrido[4,3-d][3]benzazepin-5-yl)acetamide;
Enantiomer (7S) of N-(4-chloro-3-fluoro-phenyl)-2-(9-fluoro-2,3,7-trimethyl-1,6-dioxo-7H-pyrido[4,3-d][3]benzazepin-5-yl)acetamide;
Enantiomer (7R) of N-[4-(difluoromethyl)phenyl]-2-(2,3,7-trimethyl-1,6-dioxo-7H-pyrido[4,3-d][3]benzazepin-5-yl)acetamide;
Enantiomer (7S) of N-[4-(difluoromethyl)phenyl]-2-(2,3,7-trimethyl-1,6-dioxo-7H-pyrido[4,3-d][3]benzazepin-5-yl)acetamide;
Enantiomer (10R) or (10S) of N-(4-chloro-3-fluoro-phenyl)-2-[4-ethyl-5,10-dimethyl-3,9-dioxo-4,8,12-triazatricyclo[9.4.0.0^{2,7}]pentadeca-1(15),2(7),5,11,13-pentaen-8-yl]acetamide;
Enantiomer (10S) of N-(4-chloro-3-fluoro-phenyl)-2-[4-ethyl-5,10-dimethyl-3,9-dioxo-4,8,12-triazatricyclo[9.4.0.0^{2,7}]pentadeca-1(15),2(7),5,11,13-pentaen-8-yl]acetamide;
Enantiomer (10R) of N-[4-(difluoromethyl)phenyl]-2-[4,5,10-trimethyl-3,9-dioxo-4,8,12-triazatricyclo[9.4.0.0^{2,7}]pentadeca-1(11),2(7),5,12,14-pentaen-8-yl]acetamide;
Enantiomer (10S) of N-[4-(difluoromethyl)phenyl]-2-[4,5,10-trimethyl-3,9-dioxo-4,8,12-triazatricyclo[9.4.0.0²,71pentadeca-1(11),2(7),5,12,14-pentaen-8-yl]acetamide;
Enantiomer (10R) of N-[4-(trifluoromethyl)phenyl]-2-[4,5,10-trimethyl-3,9-dioxo-4,8,12-triazatricyclo[9.4.0.0²,71pentadeca-1(15),2(7),5,11,13-pentaen-8-yl]acetamide;
Enantiomer (10S) of N-[4-(trifluoromethyl)phenyl]-2-[4,5,10-trimethyl-3,9-dioxo-4,8,12-triazatricyclo[9.4.0.0^{2,7}]pentadeca-1(15),2(7),5,11,13-pentaen-8-yl]acetamide;
N-(4-chloro-3-fluoro-phenyl)-2-(4-cyclopropyl-5,10-dimethyl-3,9-dioxo-4,8,12-triazatricyclo[9.4.0.02,7]pentadeca-1(11),2(7),5,12,14-pentaen-8-yl)acetamide;
2-(3-fluoro-5,10-dimethyl-9-oxo-4,8,14-triazatricyclo[9.4.0.02,7]pentadeca-1(11),2(7),3,5,12,14-hexaen-8-yl)-N-[4-(trifluoromethyl)phenyl]acetamide;
N-(4-chloro-3-fluoro-phenyl)-2-(9-chloro-1,3,7-trimethyl-2,6-dioxo-7H-pyrido[3,2-d][3]benzazepin-5-yl)acetamide;
2-(3-fluoro-5-methoxy-10-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
2-(3-fluoro-5-methyl-9,12-dioxo-4,8,13-triazatricyclo[9.4.0.02,7]pentadeca-1(11),2(7),3,5,14-pentaen-8-yl)-N-[4-(trifluoromethyl)phenyl]acetamide;
Enantiomer (10R) of 2-(3-fluoro-5,10-dimethyl-4,9-dioxo-5,8,12-triazatricyclo[9.4.0.02,7]pentadeca-1(11),2,6,12,14-pentaen-8-yl)-N-[4-(trifluoromethyl)phenyl]acetamide;
Enantiomer (10S) of 2-(3-fluoro-5,10-dimethyl-4,9-dioxo-5,8,12-triazatricyclo[9.4.0.02,7]pentadeca-1(11),2,6,12,14-pentaen-8-yl)-N-[4-(trifluoromethyl)phenyl]acetamide;
2-(5-fluoro-3,10-dimethyl-4,9-dioxo-3,8,12-triazatricyclo[9.4.0.02,7]pentadeca-1(11),2(7),5,12,14-pentaen-8-yl)-N-[4-(trifluoromethyl)phenyl]acetamide;
Enantiomer (7S) of N-(4-chloro-3-fluoro-phenyl)-2-[7,12-dimethyl-8-oxo-5,6,9,13-tetrazatricyclo[8.4.0.02,6]tetradeca-1(10),2,4,11,13-pentaen-9-yl]acetamide;
Enantiomer (7R) of N-(4-chloro-3-fluoro-phenyl)-2-[7,12-dimethyl-8-oxo-5,6,9,13-tetrazatricyclo[8.4.0.02,6]tetradeca-1(10),2,4,11,13-pentaen-9-yl]acetamide;
Enantiomer (10R) of 2-[3-fluoro-10-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
Enantiomer (10S) of 2-[3-fluoro-10-methyl-9-oxo-4,8,12-triazatricyclo[9.4.0.02,7pentadeca-1(11),2(7),3,5,12,14-hexaen-8-yl]-N-[4-(trifluoromethyl)phenyl]acetamide;
Enantiomer (10R) of 2-[3-chloro-10-fluoro-5-methyl-9-oxo-4,8,12-triazatricyclo[9.4.0.02,7]pentadeca-1(11),2(7),3,5,12,14-hexaen-8-yl]-N-[4-(trifluoromethyl)phenyl]acetamide;
Enantiomer (10S) of 2-[3-chloro-10-fluoro-5-methyl-9-oxo-4,8,12-triazatricyclo[9.4.0.02,7]pentadeca-1(11),2(7),3,5,12,14-hexaen-8-yl)-N-[4-(trifluoromethyl)phenyl]acetamide;
Atropisomer aR of 2-(1-chloro-3-methyl-6-oxo-7H-pyrido[4,3-d][3]benzazepin-5-yl)-N-[4-(trifluoromethyl)phenyl]acetamide; or aS of 2-(1-chloro-3-methyl-6-oxo-7H-pyrido[4,3-d][3]benzazepin-5-yl)-N-[4-(trifluoromethyl)phenyl]acetamide;
Atropisomer aS of 2-(1-chloro-3-methyl-6-oxo-7H-pyrido[4,3-d][3]benzazepin-5-yl)-N-[4-(trifluoromethyl)phenyl]acetamide: or aR of 2-(1-chloro-3-methyl-6-oxo-7H-pyrido[4,3-d][3]benzazepin-5-yl)-N-[4-(trifluoromethyl)phenyl]acetamide;
2-[(10S)-3-fluoro-5,10-dimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
Atropisomer aR of 2-(1-chloro-9-fluoro-6-oxo-7H-pyrido[4,3-d][3]benzazepin-5-yl)-N-[4-(trifluoromethyl)phenyl]acetamide;
Atropisomer aS of 2-(1-chloro-9-fluoro-6-oxo-7H-pyrido[4,3-d][3]benzazepin-5-yl)-N-[4-(trifluoromethyl)phenyl]acetamide;
Atropisomer aR of 2-[1-(difluoromethyl)-3-methyl-6-oxo-7H-pyrido[4,3-d][3]benzazepin-5-yl]-N-[4-(trifluoromethyl)phenyl]acetamide;
Atropisomer aS of 2-[1-(difluoromethyl)-3-methyl-6-oxo-7H-pyrido[4,3-d][3]benzazepin-5-yl]-N-[4-(trifluoromethyl)phenyl]acetamide;
Enantiomer (10R) of 2-(3,10-difluoro-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
Enantiomer (10S) of 2-(3,10-difluoro-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
Atropisomer aR of 2-(1-cyano-3-methyl-6-oxo-7H-pyrido[4,3-d][3]benzazepin-5-yl)-N-[4-(trifluoromethyl)phenyl]acetamide;
Atropisomer aS of 2-(1-cyano-3-methyl-6-oxo-7H-pyrido[4,3-d][3]benzazepin-5-yl)-N-[4-(trifluoromethyl)phenyl]acetamide;
Enantiomer (10R) of 2-[3-chloro-5,10-dimethyl-9-oxo-4,8,12-triazatricyclo[9.4.0.0^{2,7}]pentadeca-1(11),2,4,6,12,14-hexaen-8-yl]-N-[4-(trifluoromethyl)phenyl]acetamide;
Enantiomer (10S) of 2-[3-chloro-5,10-dimethyl-9-oxo-4,8,12-triazatricyclo[9.4.0.0^{2,7}]pentadeca-1(11),2,4,6,12,14-hexaen-8-yl]-N-[4-(trifluoromethyl)phenyl]acetamide;
Enantiomer (7R) of 2-[14-fluoro-7,12-dimethyl-8-oxo-5,6,9,13-tetrazatricyclo[8.4.0.0^{2,7}]tetradeca-1(10),2,4,11,13-pentaen-9-yl]-N-[4-(trifluoromethyl)phenyl]acetamide;
Enantiomer (7S) of 2-[14-fluoro-7,12-dimethyl-8-oxo-5,6,9,13-tetrazatricyclo[8.4.0.0^{2,7}]tetradeca-1(10),2,4,11,13-pentaen-9-yl]-N-[4-(trifluoromethyl)phenyl]acetamide;
Enantiomer (7R) of 2-[1-chloro-7-methoxy-3-methyl-6-oxo-7H-pyrido[4,3-d][3]benzazepin-5-yl]-N-[4-(trifluoromethyl)phenyl]acetamide;
Enantiomer (7S) of 2-[1-chloro-7-methoxy-3-methyl-6-oxo-7H-pyrido[4,3-d][3]benzazepin-5-yl]-N-[4-(trifluoromethyl)phenyl]acetamide;
Enantiomer (10R) of N-(4-chloro-3-fluoro-phenyl)-2-[3-fluoro-5,10-dimethyl-9-oxo-4,8,12-triazatricyclo[9.4.0.0^{2,7}]pentadeca-1(11),2(7),3,5,12,14-hexaen-8-yl]acetamide;
Enantiomer (10S) of N-(4-chloro-3-fluoro-phenyl)-2-[3-fluoro-5,10-dimethyl-9-oxo-4,8,12-triazatricyclo[9.4.0.0^{2,7}]pentadeca-1(11),2(7),3,5,12,14-hexaen-8-yl]acetamide;
Enantiomer (10R) of 2-[5,10-dimethyl-9-oxo-4,8,12-triazatricyclo[9.4.0.0^{2,7}]pentadeca-1(11),2(7),3,5,12,14-hexaen-8-yl]-N-[4-(trifluoromethyl)phenyl]acetamide;
Enantiomer (10S) of 2-[5,10-dimethyl-9-oxo-4,8,12-triazatricyclo[9.4.0.0^{2,7}]pentadeca-1(11),2(7),3,5,12,14-hexaen-8-yl]-N-[4-(trifluoromethyl)phenyl]acetamide;
Enantiomer (8R) of 2-[15-fluoro-8,13-dimethyl-9-oxo-4,6,10,14-tetrazatricyclo[9.4.0.0^{2,7}]pentadeca-1(15),2(7),3,5,11,13-hexaen-10-yl]-N-[4-(trifluoromethyl)phenyl]acetamide;
Enantiomer (8S) of 2-[15-fluoro-8,13-dimethyl-9-oxo-4,6,10,14-tetrazatricyclo[9.4.0.0^{2,7}]pentadeca-1(15),2(7),3,5,11,13-hexaen-10-yl]-N-[4-(trifluoromethyl)phenyl]acetamide;
Enantiomer (10R) of 2-[3-fluoro-5-methyl-9-oxo-10-(trideuteriomethyl)-4,8,12-triazatricyclo[9.4.0.0^{2,7}]pentadeca-1(15),2(7),3,5,11,13-hexaen-8-yl]-N-[4-(trifluoromethyl)phenyl]acetamide;
Enantiomer (10S) of 2-[3-fluoro-5-methyl-9-oxo-10-(trideuteriomethyl)-4,8,12-triazatricyclo[9.4.0.0^{2,7}]pentadeca-1(15),2(7),3,5,11,13-hexaen-8-yl]-N-[4-(trifluoromethyl)phenyl]acetamide;
Enantiomer (10R) of 2-[3-fluoro-5,10,13-trimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
Enantiomer (10S) of 2-[3-fluoro-5,10,13-trimethyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
Enantiomer (7R) of 2-[3,7-dimethyl-1,6-dioxo-2,7-dihydropyrido[4,3-d][3]benzazepin-5-yl]-N-[4-(trifluoromethyl)phenyl]acetamide;
Enantiomer (7S) of 2-[3,7-dimethyl-1,6-dioxo-2,7-dihydropyrido[4,3-d][3]benzazepin-5-yl]-N-[4-(trifluoromethyl)phenyl]acetamide;
Enantiomer (10S) of 2-[(3-fluoro-10-(hydroxymethyl)-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
Enantiomer (10R) of 2-(3-fluoro-10-(hydroxymethyl)-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
Enantiomer (8S) of 2-[15-chloro-8,13-dimethyl-9-oxo-4,6,10,14-tetrazatricyclo[9.4.0.0^{2,7}]pentadeca-1(11),2,4,6,12,14-hexaen-10-yl]-N-[4-(trifluoromethyl)phenyl]acetamide;
Enantiomer (8R) of 2-[15-chloro-8,13-dimethyl-9-oxo-4,6,10,14-tetrazatricyclo[9.4.0.0^{2,7}]pentadeca-1(11),2,4,6,12,14-hexaen-10-yl]-N-[4-(trifluoromethyl)phenyl]acetamide;
Enantiomer (10S) of N-[4-(trifluoromethyl)phenyl]-2-[3,5,10-trimethyl-4,9-dioxo-3,8,12-triazatricyclo[9.4.0.0^{2,7}]pentadeca-1(11),2(7),5,12,14-pentaen-8-yl]acetamide;
Enantiomer (10R) of N-[4-(trifluoromethyl)phenyl]-2-[3,5,10-trimethyl-4,9-dioxo-3,8,12-triazatricyclo[9.4.0.0²,71pentadeca-1(11),2(7),5,12,14-pentaen-8-yl]acetamide;
Enantiomer (10S) of 2-[3-fluoro-5-methoxy-10-methyl-9-oxo-4,8,12-triazatricyclo[9.4.0.0^{2,7}]pentadeca-1(15),2(7),3,5,11,13-hexaen-8-yl)-N-[4-(trifluoromethyl)phenyl]acetamide;
Enantiomer (10R) of 2-[3-fluoro-5-methoxy-10-methyl-9-oxo-4,8,12-triazatricyclo[9.4.0.0^{2,7}]pentadeca-1(15),2(7),3,5,11,13-hexaen-8-yl)-N-[4-(trifluoromethyl)phenyl]acetamide;
Enantiomer (10S) of 2-[3-chloro-10-methoxy-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
Enantiomer (10R) of 2-[3-chloro-10-methoxy-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
Enantiomer (10S) of 2-[3-fluoro-10-hydroxy-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
Enantiomer (10R) of 2-[3-fluoro-10-hydroxy-5-methyl-9-oxo-4,8,12-triazatricyclo[9.4.0.0^{2,7}]pentadeca-1(11),2(7),3,5,12,14-hexaen-8-yl)-N-[4-(trifluoromethyl)phenyl]acetamide;
2-(3,12-difluoro-5-methyl-9-oxo-4,8,14-triazatricyclo[9.4.0.02,7]pentadeca-1(11),2(7),3,5,12,14-hexaen-8-yl)-N-[4-(trifluoromethyl)phenyl]acetamide;
Enantiomer (10S) of N-(4-bromo-3-chloro-phenyl)-2-[(10S)-3-fluoro-10-hydroxy-5-methyl-9-oxo-4,8,12-triazatricyclo[9.4.0.02,7]pentadeca-1(11),2(7),3,5,12,14-hexaen-8-yl]acetamide;
Enantiomer (10R) of N-(4-bromo-3-chloro-phenyl)-2-[(10S)-3-fluoro-10-hydroxy-5-methyl-9-oxo-4,8,12-triazatricyclo[9.4.0.02,7]pentadeca-1(11),2(7),3,5,12,14-hexaen-8-yl]acetamide;
Enantiomer (10S) of N-[4-(difluoromethyl)phenyl]-2-[3-fluoro-5-methoxy-10-methyl-9-oxo-4,8,12-triazatricyclo[9.4.0.02,7]pentadeca-1(15),2(7),3,5,11,13-hexaen-8-yl]acetamide;
Enantiomer (10R) of N-[4-(difluoromethyl)phenyl]-2-[3-fluoro-5-methoxy-10-methyl-9-oxo-4,8,12-triazatricyclo[9.4.0.02,7]pentadeca-1(15),2(7),3,5,11,13-hexaen-8-yl]acetamide;
Enantiomer (10S) of N-(4-chloro-3-fluoro-phenyl)-2-[3-fluoro-5-methoxy-10-methyl-9-oxo-4,8,12-triazatricyclo[9.4.0.02,7]pentadeca-1(15),2(7),3,5,11,13-hexaen-8-yl]acetamide;
Enantiomer (10R) of N-(4-chloro-3-fluoro-phenyl)-2-[3-fluoro-5-methoxy-10-methyl-9-oxo-4,8,12-triazatricyclo[9.4.0.02,7]pentadeca-1(15),2(7),3,5,11,13-hexaen-8-yl]acetamide;
Enantiomer (10S) of N-(3-chloro-4-fluoro-phenyl)-2-(3-fluoro-5-methoxy-10-methyl-9-oxo-4,8,12-triazatricyclo[9.4.0.02,7]pentadeca-1(11),2(7),3,5,12,14-hexaen-8-yl)acetamide;
Enantiomer (10R) of N-(3-chloro-4-fluoro-phenyl)-2-(3-fluoro-5-methoxy-10-methyl-9-oxo-4,8,12-triazatricyclo[9.4.0.02,7]pentadeca-1(11),2(7),3,5,12,14-hexaen-8-yl)acetamide;
Enantiomer (8S) of N-(4-chloro-3-fluoro-phenyl)-2-[15-fluoro-8,13-dimethyl-9-oxo-4,6,10,14-tetrazatricyclo[9.4.0.02,7]pentadeca-1(15),2,4,6,11,13-hexaen-10-yl]acetamide;
Enantiomer (8R) of N-(4-chloro-3-fluoro-phenyl)-2-[15-fluoro-8,13-dimethyl-9-oxo-4,6,10,14-tetrazatricyclo[9.4.0.02,7]pentadeca-1(15),2,4,6,11,13-hexaen-10-yl]acetamide;
Enantiomer (8S) of (N-[4-(difluoromethyl)phenyl]-2-[15-fluoro-8,13-dimethyl-9-oxo-4,6,10,14-tetrazatricyclo[9.4.0.02,7]pentadeca-1(15),2,4,6,11,13-hexaen-10-yl]acetamide;
Enantiomer (8S) of (N-[4-(difluoromethyl)phenyl]-2-[15-fluoro-8,13-dimethyl-9-oxo-4,6,10,14-tetrazatricyclo[9.4.0.02,7]pentadeca-1(15),2,4,6,11,13-hexaen-10-yl]acetamide;
Enantiomer (7S) of N-(4-chloro-3-fluoro-phenyl)-2-[2,3,7-trimethyl-1,6-dioxo-7H-pyrido[4,3-d][3]benzazepin-5-yl]acetamide;
Enantiomer (7R) of N-(4-chloro-3-fluoro-phenyl)-2-[2,3,7-trimethyl-1,6-dioxo-7H-pyrido[4,3-d][3]benzazepin-5-yl]acetamide;
Enantiomer (8S) of 2-[15-chloro-8,13-dimethyl-9-oxo-4,6,10,14-tetrazatricyclo[9.4.0.02,7]pentadeca-1(15),2,4,6,11,13-hexaen-10-yl]-N-(4-chloro-3-fluoro-phenyl)acetamide;
Enantiomer (8R) of 2-[15-chloro-8,13-dimethyl-9-oxo-4,6,10,14-tetrazatricyclo[9.4.0.02,7]pentadeca-1(15),2,4,6,11,13-hexaen-10-yl]-N-(4-chloro-3-fluoro-phenyl)acetamide;
Enantiomer (7S) of N-[4-(difluoromethyl)phenyl]-2-[14-fluoro-7,12-dimethyl-8-oxo-5,6,9,13-tetrazatricyclo[8.4.0.02,6]tetradeca-1(14),2,4,10,12-pentaen-9-yl]acetamide;
Enantiomer (7R) of N-[4-(difluoromethyl)phenyl]-2-[14-fluoro-7,12-dimethyl-8-oxo-5,6,9,13-tetrazatricyclo[8.4.0.02,6]tetradeca-1(14),2,4,10,12-pentaen-9-yl]acetamide;
Enantiomer (10S) of N-[4-(difluoromethyl)phenyl]-2-[3-fluoro-10-(hydroxymethyl)-5-methyl-9-oxo-4,8,12-triazatricyclo[9.4.0.02,7]pentadeca-1(15),2(7),3,5,11,13-hexaen-8-yl]acetamide;
Enantiomer (10R) of N-[4-(difluoromethyl)phenyl]-2-[3-fluoro-10-(hydroxymethyl)-5-methyl-9-oxo-4,8,12-triazatricyclo[9.4.0.02,7]pentadeca-1(15),2(7),3,5,11,13-hexaen-8-yl]acetamide;
Enantiomer (10R) of 2-[3-chloro-10-fluoro-5-methyl-9-oxo-4,8,12-triazatricyclo[9.4.0.02,7]pentadeca-1(11),2(7),3,5,12,14-hexaen-8-yl)-N-[4-(difluoromethyl)phenyl]acetamide;
Enantiomer (10S) of 2-[3-chloro-10-fluoro-5-methyl-9-oxo-4,8,12-triazatricyclo[9.4.0.02,7]pentadeca-1(11),2(7),3,5,12,14-hexaen-8-yl)-N-[4-(difluoromethyl)phenyl]acetamide;
Enantiomer (10S) of N-(4-bromo-3-fluoro-phenyl)-2-[3-chloro-5,10-dimethyl-9-oxo-4,8,12-triazatricyclo[9.4.0.02,7]pentadeca-1(15),2,4,6,11,13-hexaen-8-yl]acetamide;
Enantiomer (10R) of N-(4-bromo-3-fluoro-phenyl)-2-[3-chloro-5,10-dimethyl-9-oxo-4,8,12-triazatricyclo[9.4.0.02,7]pentadeca-1(15),2,4,6,11,13-hexaen-8-yl]acetamide;
Enantiomer (10S) of N-[2-fluoro-4-(trifluoromethyl)phenyl]-2-[3-chloro-5,10-dimethyl-9-oxo-4,8,12-triazatricyclo[9.4.0.02,7]pentadeca-1(11),2(7),3,5,12,14-hexaen-8-yl]acetamide;
Enantiomer (10R) of N-[2-fluoro-4-(trifluoromethyl)phenyl]-2-[3-chloro-5,10-dimethyl-9-oxo-4,8,12-triazatricyclo[9.4.0.02,7]pentadeca-1(11),2(7),3,5,12,14-hexaen-8-yl]acetamide;
Enantiomer (10S) of N-(4-chloro-2,5-difluoro-phenyl)-2-[3-chloro-5,10-dimethyl-9-oxo-4,8,12-triazatricyclo[9.4.0.02,7]pentadeca-1(11),2(7),3,5,12,14-hexaen-8-yl]acetamide;
Enantiomer (10R) of N-(4-chloro-2,5-difluoro-phenyl)-2-[3-chloro-5,10-dimethyl-9-oxo-4,8,12-triazatricyclo[9.4.0.02,7]pentadeca-1(11),2(7),3,5,12,14-hexaen-8-yl]acetamide;
Enantiomer (10S) of N-(4-bromo-2-fluoro-phenyl)-2-[3-chloro-5,10-dimethyl-9-oxo-4,8,12-triazatricyclo[9.4.0.02,7]pentadeca-1(11),2(7),3,5,12,14-hexaen-8-yl]acetamide;
Enantiomer (10R) of N-(4-bromo-2-fluoro-phenyl)-2-[3-chloro-5,10-dimethyl-9-oxo-4,8,12-triazatricyclo[9.4.0.02,7]pentadeca-1(11),2(7),3,5,12,14-hexaen-8-yl]acetamide;
Enantiomer (10S) of 2-[3-chloro-5,10-dimethyl-9-oxo-4,8,12-triazatricyclo[9.4.0.02,7]pentadeca-1(15),2,4,6,11,13-hexaen-8-yl]-N-[6-(trifluoromethyl)-3-pyridyl]acetamide;
Enantiomer (10R) of 2-[3-chloro-5,10-dimethyl-9-oxo-4,8,12-triazatricyclo[9.4.0.02,7]pentadeca-1(15),2,4,6,11,13-hexaen-8-yl]-N-[6-(trifluoromethyl)-3-pyridyl]acetamide;
Enantiomer (10S) of 2-[3-chloro-5,10-dimethyl-9-oxo-4,8,12-triazatricyclo[9.4.0.02,7]pentadeca-1(15),2,4,6,11,13-hexaen-8-yl]-N-[5-(trifluoromethyl)-2-pyridyl]acetamide;
Enantiomer (10R) of 2-[3-chloro-5,10-dimethyl-9-oxo-4,8,12-triazatricyclo[9.4.0.02,7]pentadeca-1(15),2,4,6,11,13-hexaen-8-yl]-N-[5-(trifluoromethyl)-2-pyridyl]acetamide;
Enantiomer (10R) of 2-[3-chloro-5,10-dimethyl-9-oxo-4,8,12-triazatricyclo[9.4.0.02,7]pentadeca-1(11),2(7),3,5,12,14-hexaen-8-yl)-N-[6-(difluoromethyl)-3-pyridyl]acetamide;
Enantiomer (10S) of 2-[3-chloro-5,10-dimethyl-9-oxo-4,8,12-triazatricyclo[9.4.0.02,7]pentadeca-1(11),2(7),3,5,12,14-hexaen-8-yl]-N-[6-(difluoromethyl)-3-pyridyl]acetamide;
2-[(10R)-3-fluoro-5,10-dimethyl-9-oxo-4,8,12-triazatricyclo[9.4.0.02,7]pentadeca-1(15),2,4,6,11,13-hexaen-8-yl]-N-(4-fluorophenyl)acetamide;
N-(4-bromo-3-fluoro-phenyl)-2-[(10R)-3-fluoro-5,10-dimethyl-9-oxo-4,8,12-triazatricyclo[9.4.0.02,7]pentadeca-1(15),2,4,6,11,13-hexaen-8-yl]acetamide;
N-[4-(difluoromethyl)-3-fluoro-phenyl]-2-[(10R)-3-fluoro-5,10-dimethyl-9-oxo-4,8,12-triazatricyclo[9.4.0.02,7]pentadeca-1(15),2,4,6,11,13-hexaen-8-yl]acetamide;
N-[4-(1,1-difluoroethyl)-3-fluoro-phenyl]-2-[(10R)-3-fluoro-5,10-dimethyl-9-oxo-4,8,12-triazatricyclo[9.4.0.02,7]pentadeca-1(15),2,4,6,11,13-hexaen-8-yl]acetamide;
N-(2,2-difluoroindan-5-yl)-2-[(10R)-3-fluoro-5,10-dimethyl-9-oxo-4,8,12-triazatricyclo[9.4.0.02,7]pentadeca-1(15),2,4,6,11,13-hexaen-8-yl]acetamide;
N-(2,2-difluoro-1,3-benzodioxol-5-yl)-2-[(10R)-3-fluoro-5,10-dimethyl-9-oxo-4,8,12-triazatricyclo[9.4.0.02,7]pentadeca-1(15),2,4,6,11,13-hexaen-8-yl]acetamide;
2-[(10R)-3-fluoro-5,10-dimethyl-9-oxo-4,8,12-triazatricyclo[9.4.0.02,7]pentadeca-1(15),2,4,6,11,13-hexaen-8-yl]-N-(3-fluoro-6-isoquinolyl)acetamide;
N-(1-chloro-6-isoquinolyl)-2-[(10R)-3-fluoro-5,10-dimethyl-9-oxo-4,8,12-triazatricyclo[9.4.0.02,7]pentadeca-1(15),2,4,6,11,13-hexaen-8-yl]acetamide;
N-(3-chloro-4-fluoro-phenyl)-2-[(10R)-3-fluoro-5,10-dimethyl-9-oxo-4,8,12-triazatricyclo[9.4.0.02,7]pentadeca-1(15),2,4,6,11,13-hexaen-8-yl]acetamide;
N-(4-chloro-2-fluoro-phenyl)-2-[(10R)-3-fluoro-5,10-dimethyl-9-oxo-4,8,12-triazatricyclo[9.4.0.02,7]pentadeca-1(15),2,4,6,11,13-hexaen-8-yl]acetamide;
N-(5-chloro-3-thienyl)-2-[(10R)-3-fluoro-5,10-dimethyl-9-oxo-4,8,12-triazatricyclo[9.4.0.02,7]pentadeca-1(11),2(7),3,5,12,14-hexaen-8-yl]acetamide;
2-[(10R)-3-fluoro-5,10-dimethyl-9-oxo-4,8,12-triazatricyclo[9.4.0.02,7]pentadeca-1(11),2(7),3,5,12,14-hexaen-8-yl]-N-(3-fluoro-4-isopropyl-phenyl)acetamide;
N-(4-chloro-2,5-difluoro-phenyl)-2-[(10R)-3-fluoro-5,10-dimethyl-9-oxo-4,8,12-triazatricyclo[9.4.0.02,7]pentadeca-1(15),2,4,6,11,13-hexaen-8-yl]acetamide;
N-[4-(1-fluorocyclopropyl)phenyl]-2-[(10R)-3-fluoro-5,10-dimethyl-9-oxo-4,8,12-triazatricyclo[9.4.0.02,7]pentadeca-1(11),2(7),3,5,12,14-hexaen-8-yl]acetamide;
2-[(10R)-3-fluoro-5,10-dimethyl-9-oxo-4,8,12-triazatricyclo[9.4.0.02,7]pentadeca-1(11),2(7),3,5,12,14-hexaen-8-yl]-N-[2-fluoro-4-(trifluoromethyl)phenyl]acetamide;
2-[(10R)-3-fluoro-5,10-dimethyl-9-oxo-4,8,12-triazatricyclo[9.4.0.02,7]pentadeca-1(11),2(7),3,5,12,14-hexaen-8-yl)-N-[3-fluoro-4-(1-fluorocyclopropyl)phenyl]acetamide;
N-(4-chloro-2,3-difluoro-phenyl)-2-[(10R)-3-fluoro-5,10-dimethyl-9-oxo-4,8,12-triazatricyclo[9.4.0.02,7]pentadeca-1(15),2,4,6,11,13-hexaen-8-yl]acetamide;
Enantiomer (10S) of N-[4-(difluoromethyl)phenyl]-2-[5,10-dimethyl-9-oxo-4,8,12-triazatricyclo[9.4.0.02,7]pentadeca-1(15),2,4,6,11,13-hexaen-8-yl]acetamide;
Enantiomer (10R) of N-[4-(difluoromethyl)phenyl]-2-[5,10-dimethyl-9-oxo-4,8,12-triazatricyclo[9.4.0.02,7]pentadeca-1(15),2,4,6,11,13-hexaen-8-yl]acetamide;
Enantiomer (10S) of N-(4-chloro-3-fluoro-phenyl)-2-[5,10-dimethyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[5,10-dimethyl-9-oxo-4,8,12-triazatricyclo[9.4.0.02,7]pentadeca-1(15),2,4,6,11,13-hexaen-8-yl]acetamide;
Enantiomer (10S) of N-[4-(difluoromethyl)-3-fluoro-phenyl]-2-[5,10-dimethyl-9-oxo-4,8,12-triazatricyclo[9.4.0.02,7]pentadeca-1(15),2,4,6,11,13-hexaen-8-yl]acetamide;
Enantiomer (10R) of N-[4-(difluoromethyl)-3-fluoro-phenyl]-2-[5,10-dimethyl-9-oxo-4,8,12-triazatricyclo[9.4.0.02,7]pentadeca-1(15),2,4,6,11,13-hexaen-8-yl]acetamide;
Enantiomer (10R) of N-(3-chloro-4-fluoro-phenyl)-2-(5,10-dimethyl-9-oxo-4,8,12-triazatricyclo[9.4.0.02,7]pentadeca-1(11),2(7),3,5,12,14-hexaen-8-yl)acetamide;
Enantiomer (10S) of N-(3-chloro-4-fluoro-phenyl)-2-(5,10-dimethyl-9-oxo-4,8,12-triazatricyclo[9.4.0.02,7]pentadeca-1(11),2(7),3,5,12,14-hexaen-8-yl)acetamide;
N-(4-chloro-3-fluoro-phenyl)-2-(4,5,10-trimethyl-3,9-dioxo-4,8,12-triazatricyclo[9.4.0.02,7]pentadeca-1(15),2(7),5,11,13-pentaen-8-yl)acetamide;
N-[4-(difluoromethyl)phenyl]-2-(9-fluoro-2,3,7-trimethyl-1,6-dioxo-7H-pyrido[4,3-d][3]benzazepin-5-yl)acetamide;
Enantiomer (10S) of N-[4-(difluoromethyl)phenyl]-2-[3-fluoro-5,10-dimethyl-9-oxo-4,8,12,13-tetrazatricyclo[9.4.0.02,7]pentadeca-1(15),2,4,6,11,13-hexaen-8-yl]acetamide;
Enantiomer (10R) of N-[4-(difluoromethyl)phenyl]-2-[3-fluoro-5,10-dimethyl-9-oxo-4,8,12,13-tetrazatricyclo[9.4.0.02,7]pentadeca-1(15),2,4,6,11,13-hexaen-8-yl]acetamide;
Enantiomer (10S) of 2-[3-chloro-5,10-dimethyl-9-oxo-4,8,12,13-tetrazatricyclo[9.4.0.02,7]pentadeca-1(15),2,4,6,11,13-hexaen-8-yl]-N-(4-chloro-3-fluoro-phenyl)acetamide;
Enantiomer (10R) of 2-[3-chloro-5,10-dimethyl-9-oxo-4,8,12,13-tetrazatricyclo[9.4.0.02,7]pentadeca-1(15),2,4,6,11,13-hexaen-8-yl]-N-(4-chloro-3-fluoro-phenyl)acetamide;
Enantiomer (10S) of N-[4-(difluoromethyl)phenyl]-2-[3-fluoro-5,10-dimethyl-9-oxo-4,8,14-triazatricyclo[9.4.0.02,7]pentadeca-1(15),2,4,6,11,13-hexaen-8-yl]acetamide;
Enantiomer (10R) of N-[4-(difluoromethyl)phenyl]-2-[3-fluoro-5,10-dimethyl-9-oxo-4,8,14-triazatricyclo[9.4.0.02,7]pentadeca-1(15),2,4,6,11,13-hexaen-8-yl]acetamide;
N-(4-chloro-3-fluoro-phenyl)-2-[(10S)-3,14-difluoro-5,10-dimethyl-9-oxo-4,8,12-triazatricyclo[9.4.0.02,7]pentadeca-1(11),2(7),3,5,12,14-hexaen-8-yl]acetamide;
N-(4-chloro-3-fluoro-phenyl)-2-[(10R)-3,14-difluoro-5,10-dimethyl-9-oxo-4,8,12-triazatricyclo[9.4.0.02,7]pentadeca-1(11),2(7),3,5,12,14-hexaen-8-yl]acetamide;
Enantiomer (10S) of N-[4-(difluoromethyl)phenyl]-2-[3,14-difluoro-5,10-dimethyl-9-oxo-4,8,12-triazatricyclo[9.4.0.02,7]pentadeca-1(11),2(7),3,5,12,14-hexaen-8-yl]acetamide;
Enantiomer (10R) of N-[4-(difluoromethyl)phenyl]-2-[3,14-difluoro-5,10-dimethyl-9-oxo-4,8,12-triazatricyclo[9.4.0.02,7]pentadeca-1(11),2(7),3,5,12,14-hexaen-8-yl]acetamide;
Enantiomer (10S) of 2-[3,14-difluoro-5,10-dimethyl-9-oxo-4,8,12-triazatricyclo[9.4.0.02,7]pentadeca-1(11),2(7),3,5,12,14-hexaen-8-yl)-N-[6-(trifluoromethyl)-3-pyridyl]acetamide;
Enantiomer (10R) of 2-[3,14-difluoro-5,10-dimethyl-9-oxo-4,8,12-triazatricyclo[9.4.0.02,7]pentadeca-1(11),2(7),3,5,12,14-hexaen-8-yl)-N-[6-(trifluoromethyl)-3-pyridyl]acetamide;
Enantiomer (10S) of 2-[3,14-difluoro-5,10-dimethyl-9-oxo-4,8,12-triazatricyclo[9.4.0.02,7]pentadeca-1(15),2,4,6,11,13-hexaen-8-yl]-N-[4-(1,1-difluoroethyl)phenyl]acetamide;
Enantiomer (10R) of 2-[3,14-difluoro-5,10-dimethyl-9-oxo-4,8,12-triazatricyclo[9.4.0.02,7]pentadeca-1(15),2,4,6,11,13-hexaen-8-yl]-N-[4-(1,1-difluoroethyl)phenyl]acetamide;
N-(4-chloro-3-fluoro-phenyl)-2-[3-fluoro-5-[2-methoxyethyl(methyl)aminol-10-methyl-9-oxo-4,8,12-triazatricyclo[9.4.0.02,7]pentadeca-1(15),2(7),3,5,11,13-hexaen-8-yl]acetamide;
Enantiomer (10S) of N-(4-chloro-3-fluoro-phenyl)-2-[3-fluoro-5-[2-methoxyethyl(methyl)aminol-10-methyl-9-oxo-4,8,12-triazatricyclo[9.4.0.02,7]pentadeca-1(15),2(7),3,5,11,13-hexaen-8-yl]acetamide;
Enantiomer (10R) of N-(4-chloro-3-fluoro-phenyl)-2-[3-fluoro-5-[2-methoxyethyl(methyl)aminol-10-methyl-9-oxo-4,8,12-triazatricyclo[9.4.0.02,7]pentadeca-1(15),2(7),3,5,11,13-hexaen-8-yl]acetamide;
Enantiomer (10S) of N-(4-chloro-3-fluoro-phenyl)-2-[5-[2-(dimethylamino)ethyl-methyl-aminol-3-fluoro-10-methyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[5-[2-(dimethylamino)ethyl-methyl-aminol-3-fluoro-10-methyl-9-oxo-4,8,12-triazatricyclo[9.4.0.02,7]pentadeca-1(15),2,4,6,11,13-hexaen-8-yl]acetamide;
Enantiomer (10S) of 2-[5-(5-azaspiro[2.31hexan-5-yl)-3-fluoro-10-methyl-9-oxo-4,8,12-triazatricyclo[9.4.0.02,7]pentadeca-1(15),2,4,6,11,13-hexaen-8-yl]-N-(4-chloro-3-fluoro-phenyl)acetamide;
Enantiomer (10R) of 2-[5-(5-azaspiro[2.31hexan-5-yl)-3-fluoro-10-methyl-9-oxo-4,8,12-triazatricyclo[9.4.0.02,7]pentadeca-1(15),2,4,6,11,13-hexaen-8-yl]-N-(4-chloro-3-fluoro-phenyl)acetamide;
Enantiomer (10S) of N-(4-chloro-3-fluoro-phenyl)-2-[5-(3,3-difluoroazetidin-1-yl)-3-fluoro-10-methyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[5-(3,3-difluoroazetidin-1-yl)-3-fluoro-10-methyl-9-oxo-4,8,12-triazatricyclo[9.4.0.02,7]pentadeca-1(15),2,4,6,11,13-hexaen-8-yl]acetamide;
Enantiomer (10S) of 2-[5-(6-azabicyclo[3.1.1]heptan-6-yl)-3-fluoro-10-methyl-9-oxo-4,8,12-triazatricyclo[9.4.0.02,7]pentadeca-1(11),2(7),3,5,12,14-hexaen-8-yl]-N-(4-chloro-3-fluoro-phenyl)acetamide;
Enantiomer (10R) of 2-[5-(6-azabicyclo[3.1.11heptan-6-yl)-3-fluoro-10-methyl-9-oxo-4,8,12-triazatricyclo[9.4.0.02,7]pentadeca-1(11),2(7),3,5,12,14-hexaen-8-yl]-N-(4-chloro-3-fluoro-phenyl)acetamide;
Enantiomer (10S) of N-(4-chloro-3-fluoro-phenyl)-2-[5-(2,2-difluoro-5-azaspiro[2.31hexan-5-yl)-3-fluoro-10-methyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[5-(2,2-difluoro-5-azaspiro[2.31hexan-5-yl)-3-fluoro-10-methyl-9-oxo-4,8,12-triazatricyclo[9.4.0.02,7]pentadeca-1(15),2,4,6,11,13-hexaen-8-yl]acetamide;
N-[4-(difluoromethyl)phenyl]-2-(3-fluoro-10-methyl-5-morpholino-9-oxo-4,8,12-triazatricyclo[9.4.0.02,7]pentadeca-1(15),2,4,6,11,13-hexaen-8-yl)acetamide;
N-[4-(difluoromethyl)phenyl]-2-[3-fluoro-5-[(2S)-2-(hydroxymethyl)azetidin-1-yl]-10-methyl-9-oxo-4,8,12-triazatricyclo[9.4.0.02,7]pentadeca-1(15),2,4,6,11,13-hexaen-8-yl]acetamide;
N-[4-(difluoromethyl)phenyl]-2-[3-fluoro-5-[(2R)-2-(hydroxymethyl)azetidin-1-yl]-10-methyl-9-oxo-4,8,12-triazatricyclo[9.4.0.02,7]pentadeca-1(15),2,4,6,11,13-hexaen-8-yl]acetamide;
Enantiomer (10S) of N-[4-(difluoromethyl)phenyl]-2-[3-fluoro-5-(3-fluoroazetidin-1-yl)-10-methyl-9-oxo-4,8,12-triazatricyclo[9.4.0.02,7]pentadeca-1(15),2,4,6,11,13-hexaen-8-yl]acetamide;
Enantiomer (10R) of N-[4-(difluoromethyl)phenyl]-2-[3-fluoro-5-(3-fluoroazetidin-1-yl)-10-methyl-9-oxo-4,8,12-triazatricyclo[9.4.0.02,7]pentadeca-1(15),2,4,6,11,13-hexaen-8-yl]acetamide;
Enantiomer (10S) of N-[4-(difluoromethyl)phenyl]-2-[3-fluoro-5-(3-hydroxy-3-methyl-azetidin-1-yl)-10-methyl-9-oxo-4,8,12-triazatricyclo[9.4.0.02,7]pentadeca-1(15),2,4,6,11,13-hexaen-8-yl]acetamide;
(10R) enantiomer of N-[4-(difluoromethyl)phenyl]-2-[3-fluoro-5-(3-hydroxy-3-methyl-azetidin-1-yl)-10-methyl-9-oxo-4,8,12-triazatricyclo[9.4.0.02,7]pentadeca-1(15),2,4,6,11,13-hexaen-8-yl]acetamide;
Enantiomer (10S,3S) of N-[4-(difluoromethyl)phenyl]-2-[3-fluoro-5-[3-fluoropyrrolidin-1-yl]-10-methyl-9-oxo-4,8,12-triazatricyclo[9.4.0.02,7]pentadeca-1(15),2,4,6,11,13-hexaen-8-yl]acetamide;
(10S,3R) enantiomer of N-[4-(difluoromethyl)phenyl]-2-[3-fluoro-5-[3-fluoropyrrolidin-1-yl]-10-methyl-9-oxo-4,8,12-triazatricyclo[9.4.0.02,7]pentadeca-1(15),2,4,6,11,13-hexaen-8-yl]acetamide;
Enantiomer (10R,3S) of N-[4-(difluoromethyl)phenyl]-2-[3-fluoro-5-[3-fluoropyrrolidin-1-yl]-10-methyl-9-oxo-4,8,12-triazatricyclo[9.4.0.02,7]pentadeca-1(15),2,4,6,11,13-hexaen-8-yl]acetamide;
(10R,3R) enantiomer of N-[4-(difluoromethyl)phenyl]-2-[3-fluoro-5-[3-fluoropyrrolidin-1-yl]-10-methyl-9-oxo-4,8,12-triazatricyclo[9.4.0.02,7]pentadeca-1(15),2,4,6,11,13-hexaen-8-yl]acetamide;
Enantiomer (10S) of N-[4-(difluoromethyl)phenyl]-2-[5-(3,3-difluoropyrrolidin-1-yl)-3-fluoro-10-methyl-9-oxo-4,8,12-triazatricyclo[9.4.0.02,7]pentadeca-1(15),2,4,6,11,13-hexaen-8-yl]acetamide;
Enantiomer (10R) of N-[4-(difluoromethyl)phenyl]-2-[5-(3,3-difluoropyrrolidin-1-yl)-3-fluoro-10-methyl-9-oxo-4,8,12-triazatricyclo[9.4.0.02,7]pentadeca-1(15),2,4,6,11,13-hexaen-8-yl]acetamide;
Enantiomer (10S,3S) of N-[4-(difluoromethyl)phenyl]-2-[3-fluoro-5-[3-hydroxypyrrolidin-1-yl]-10-methyl-9-oxo-4,8,12-triazatricyclo[9.4.0.02,7]pentadeca-1(15),2,4,6,11,13-hexaen-8-yl]acetamide;
Enantiomer (10S,3R) of N-[4-(difluoromethyl)phenyl]-2-[3-fluoro-5-[3-hydroxypyrrolidin-1-yl]-10-methyl-9-oxo-4,8,12-triazatricyclo[9.4.0.02,7]pentadeca-1(15),2,4,6,11,13-hexaen-8-yl]acetamide;
Enantiomer (10S) of 2-[5-(3,3-difluoroazetidin-1-yl)-3-fluoro-10-methyl-9-oxo-4,8,12-triazatricyclo[9.4.0.02,7]pentadeca-1(11),2(7),3,5,12,14-hexaen-8-yl]-N-[4-(difluoromethyl)phenyl]acetamide;
Enantiomer (10R) of 2-[5-(3,3-difluoroazetidin-1-yl)-3-fluoro-10-methyl-9-oxo-4,8,12-triazatricyclo[9.4.0.02,7]pentadeca-1(11),2(7),3,5,12,14-hexaen-8-yl]-N-[4-(difluoromethyl)phenyl]acetamide;
Enantiomer (10S) of 2-[5-(6,6-difluoro-2-azaspiro[3.31heptan-2-yl)-3-fluoro-10-methyl-9-oxo-4,8,12-triazatricyclo[9.4.0.02,7]pentadeca-1(15),2,4,6,11,13-hexaen-8-yl]-N-[4-(difluoromethyl)phenyl]acetamide;
Enantiomer (10R) of 2-[5-(6,6-difluoro-2-azaspiro[3.31heptan-2-yl)-3-fluoro-10-methyl-9-oxo-4,8,12-triazatricyclo[9.4.0.02,7]pentadeca-1(15),2,4,6,11,13-hexaen-8-yl)-N-[4-(difluoromethyl)phenyl]acetamide;
Enantiomer (10S) of N-[4-(difluoromethyl)phenyl]-2-[3-fluoro-10-methyl-5-(6-oxa-2-azaspiro[3.4]octan-2-yl)-9-oxo-4,8,12-triazatricyclo[9.4.0.02,7]pentadeca-1(15),2,4,6,11,13-hexaen-8-yl]acetamide;
Enantiomer (10R) of N-[4-(difluoromethyl)phenyl]-2-[3-fluoro-10-methyl-5-(6-oxa-2-azaspiro[3.4]octan-2-yl)-9-oxo-4,8,12-triazatricyclo[9.4.0.02,7]pentadeca-1(15),2,4,6,11,13-hexaen-8-yl]acetamide;
Enantiomer (10S) of 2-[5-(5-azaspiro[2.31hexan-5-yl)-3-fluoro-10-methyl-9-oxo-4,8,12-triazatricyclo[9.4.0.02,7]pentadeca-1(15),2,4,6,11,13-hexaen-8-yl)-N-[4-(difluoromethyl)phenyl]acetamide;
Enantiomer (10R) of 2-[5-(5-azaspiro[2.31hexan-5-yl)-3-fluoro-10-methyl-9-oxo-4,8,12-triazatricyclo[9.4.0.02,7]pentadeca-1(15),2,4,6,11,13-hexaen-8-yl)-N-[4-(difluoromethyl)phenyl]acetamide;
Enantiomer (10S) of N-[4-(difluoromethyl)phenyl]-2-[3-fluoro-10-methyl-5-(2-oxa-6-azaspiro[3.3]heptan-6-yl)-9-oxo-4,8,12-triazatricyclo[9.4.0.02,7]pentadeca-1(15),2,4,6,11,13-hexaen-8-yl]acetamide;
Enantiomer (10R) of N-[4-(difluoromethyl)phenyl]-2-[3-fluoro-10-methyl-5-(2-oxa-6-azaspiro[3.3]heptan-6-yl)-9-oxo-4,8,12-triazatricyclo[9.4.0.02,7]pentadeca-1(15),2,4,6,11,13-hexaen-8-yl]acetamide;
Enantiomer (10R,3S) of N-[4-(difluoromethyl)phenyl]-2-[3-fluoro-5-[3-hydroxypyrrolidin-1-yl]-10-methyl-9-oxo-4,8,12-triazatricyclo[9.4.0.02,7]pentadeca-1(15),2,4,6,11,13-hexaen-8-yl]acetamide;
(10R,3R) enantiomer of N-[4-(difluoromethyl)phenyl]-2-[3-fluoro-5-[3-hydroxypyrrolidin-1-yl]-10-methyl-9-oxo-4,8,12-triazatricyclo[9.4.0.02,7]pentadeca-1(15),2,4,6,11,13-hexaen-8-yl]acetamide;
Enantiomer (10S) of 2-[5-(2,2-difluoro-5-azaspiro[2.3]hexan-5-yl)-3-fluoro-10-methyl-9-oxo-4,8,12-triazatricyclo[9.4.0.02,7]pentadeca-1(15),2,4,6,11,13-hexaen-8-yl]-N-[4-(difluoromethyl)phenyl]acetamide;
Enantiomer (10R) of 2-[5-(2,2-difluoro-5-azaspiro[2.31hexan-5-yl)-3-fluoro-10-methyl-9-oxo-4,8,12-triazatricyclo[9.4.0.02,7]pentadeca-1(15),2,4,6,11,13-hexaen-8-yl]-N-[4-(difluoromethyl)phenyl]acetamide;
Enantiomer (10S) of 2-[5-(6-azabicyclo[3.1.1]heptan-6-yl)-3-fluoro-10-methyl-9-oxo-4,8,12-triazatricyclo[9.4.0.02,7]pentadeca-1(15),2,4,6,11,13-hexaen-8-yl)-N-[4-(difluoromethyl)phenyl]acetamide;
Enantiomer (10R) of 2-[5-(6-azabicyclo[3.1.11heptan-6-yl)-3-fluoro-10-methyl-9-oxo-4,8,12-triazatricyclo[9.4.0.02,7]pentadeca-1(15),2,4,6,11,13-hexaen-8-yl)-N-[4-(difluoromethyl)phenyl]acetamide;
Enantiomer (10S) of N-[4-(difluoromethyl)phenyl]-2-[3-fluoro-5-(3-fluoro-3-methyl-azetidin-1-yl)-10-methyl-9-oxo-4,8,12-triazatricyclo[9.4.0.02,7]pentadeca-1(15),2,4,6,11,13-hexaen-8-yl]acetamide;
Enantiomer (10R) of N-[4-(difluoromethyl)phenyl]-2-[3-fluoro-5-(3-fluoro-3-methyl-azetidin-1-yl)-10-methyl-9-oxo-4,8,12-triazatricyclo[9.4.0.02,7]pentadeca-1(15),2,4,6,11,13-hexaen-8-yl]acetamide;
Enantiomer (10S) of 2-[5-(6,6-difluoro-3-azabicyclo[3.1.0]hexan-3-yl)-3-fluoro-10-methyl-9-oxo-4,8,12-triazatricyclo[9.4.0.02,7]pentadeca-1(11),2(7),3,5,12,14-hexaen-8-yl]-N-[4-(difluoromethyl)phenyl]acetamide;
Enantiomer (10R) of 2-[5-(6,6-difluoro-3-azabicyclo[3.1.0]hexan-3-yl)-3-fluoro-10-methyl-9-oxo-4,8,12-triazatricyclo[9.4.0.02,7]pentadeca-1(11),2(7),3,5,12,14-hexaen-8-yl]N-[4-(difluoromethyl)phenyl]acetamide;
Enantiomer (10S) of 2-[5-(6,6-difluoro-3-azabicyclo[3.1.1]heptan-3-yl)-3-fluoro-10-methyl-9-oxo-4,8,12-triazatricyclo[9.4.0.02,7]pentadeca-1(11),2(7),3,5,12,14-hexaen-8-vyl-N-[4-(difluoromethyl)phenyl]acetamide;
Enantiomer (10R) of 2-[5-(6,6-difluoro-3-azabicyclo[3.1.11heptan-3-yl)-3-fluoro-10-methyl-9-oxo-4,8,12-triazatricyclo[9.4.0.02,7]pentadeca-1(11),2(7),3,5,12,14-hexaen-8-yl]-N-[4-(difluoromethyl)phenyl]acetamide;
Enantiomer (7R) of 2-[3-(3,3-difluoroazetidin-1-yl)-1,9-difluoro-7-methyl-6-oxo-7H-pyrido[4,3-d][3]benzazepin-5-yl]-N-[4-(difluoromethyl)phenyl]acetamide;
Enantiomer (7S) of 2-[3-(3,3-difluoroazetidin-1-yl)-1,9-difluoro-7-methyl-6-oxo-7H-pyrido[4,3-d][3]benzazepin-5-yl]-N-[4-(difluoromethyl)phenyl]acetamide;
2-[3-chloro-5-(3,3-difluoroazetidin-1-yl)-10-methyl-9-oxo-4,8,12-triazatricyclo[9.4.0.02,7]pentadeca-1(15),2,4,6,11,13-hexaen-8-yl]-N-[4-(difluoromethyl)phenyl]acetamide;
Enantiomer (10S) of N-(4-chloro-3-fluoro-phenyl)-2-[5-(3,3-difluoroazetidin-1-yl)-10-methyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[5-(3,3-difluoroazetidin-1-yl)-10-methyl-9-oxo-4,8,12-triazatricyclo[9.4.0.02,7]pentadeca-1(15),2,4,6,11,13-hexaen-8-yl]acetamide;
2-[5-(5-azaspiro[2.31hexan-5-yl)-3-fluoro-9-oxo-4,8,12-triazatricyclo[9.4.0.02,7]pentadeca-1(15),2,4,6,11,13-hexaen-8-yl]-N-[4-(difluoromethyl)phenyl]acetamide;
2-[5-(3,3-difluoroazetidin-1-yl)-3-fluoro-9-oxo-4,8,12-triazatricyclo[9.4.0.02,7]pentadeca-1(15),2,4,6,11,13-hexaen-8-yl]-N-[4-(difluoromethyl)phenyl]acetamide;
2-[3-(3,3-difluoroazetidin-1-yl)-1,9-difluoro-6-oxo-7H-pyrido[4,3-d][3]benzazepin-5-yl]-N-[4-(difluoromethyl)phenyl]acetamide;
Enantiomer (10S) of N-(4-chloro-3-fluoro-phenyl)-2-[3-methoxy-10-methyl-9-oxo-4,8,12-triazatricyclo[9.4.0.02,7]pentadeca-1(15),2(7),3,5,11,13-hexaen-8-yl]acetamide;
Enantiomer (10R) of N-(4-chloro-3-fluoro-phenyl)-2-[3-methoxy-10-methyl-9-oxo-4,8,12-triazatricyclo[9.4.0.02,7]pentadeca-1(15),2(7),3,5,11,13-hexaen-8-yl]acetamide;
Enantiomer (10S) of N-[4-(difluoromethyl)phenyl]-2-[3-methoxy-10-methyl-9-oxo-4,8,12-triazatricyclo[9.4.0.02,7]pentadeca-1(15),2(7),3,5,11,13-hexaen-8-yl]acetamide;
Enantiomer (10R) of N-[4-(difluoromethyl)phenyl]-2-[3-methoxy-10-methyl-9-oxo-4,8,12-triazatricyclo[9.4.0.02,7]pentadeca-1(15),2(7),3,5,11,13-hexaen-8-yl]acetamide;
Enantiomer (10S) of N-[4-(difluoromethyl)phenyl]-2-[5-methoxy-3,10-dimethyl-9-oxo-4,8,12-triazatricyclo[9.4.0.02,7]pentadeca-1(15),2(7),3,5,11,13-hexaen-8-yl]acetamide;
Enantiomer (10R) of N-[4-(difluoromethyl)phenyl]-2-[5-methoxy-3,10-dimethyl-9-oxo-4,8,12-triazatricyclo[9.4.0.02,7]pentadeca-1(15),2(7),3,5,11,13-hexaen-8-yl]acetamide;
Enantiomer (10S) of N-(4-chloro-3-fluoro-phenyl)-2-[5-methoxy-3,10-dimethyl-9-oxo-4,8,12-triazatricyclo[9.4.0.02,7]pentadeca-1(15),2(7),3,5,11,13-hexaen-8-yl]acetamide;
Enantiomer (10R) of N-(4-chloro-3-fluoro-phenyl)-2-[5-methoxy-3,10-dimethyl-9-oxo-4,8,12-triazatricyclo[9.4.0.02,7]pentadeca-1(15),2(7),3,5,11,13-hexaen-8-yl]acetamide;
Enantiomer (10S) of 2-[3-(difluoromethyl)-5,10-dimethyl-9-oxo-4,8,12-triazatricyclo[9.4.0.02,7]pentadeca-1(11),2(7),3,5,12,14-hexaen-8-yl]-N-[4-(difluoromethyl)phenyl]acetamide;
Enantiomer (10R) of 2-[3-(difluoromethyl)-5,10-dimethyl-9-oxo-4,8,12-triazatricyclo[9.4.0.02,7]pentadeca-1(11),2(7),3,5,12,14-hexaen-8-yl]-N-[4-(difluoromethyl)phenyl]acetamide;
Enantiomer (10S) of N-(4-chloro-3-fluoro-phenyl)-2-[3-(difluoromethyl)-5,10-dimethyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[3-(difluoromethyl)-5,10-dimethyl-9-oxo-4,8,12-triazatricyclo[9.4.0.02,7]pentadeca-1(15),2,4,6,11,13-hexaen-8-yl]acetamide;
Enantiomer (8S) of N-(4-chloro-3-fluoro-phenyl)-2-[5,15-difluoro-8,13-dimethyl-9-oxo-3,10,14-triazatricyclo[9.4.0.02,7]pentadeca-1(15),2,4,6,11,13-hexaen-10-yl]acetamide;
Enantiomer (8R) of N-(4-chloro-3-fluoro-phenyl)-2-[5,15-difluoro-8,13-dimethyl-9-oxo-3,10,14-triazatricyclo[9.4.0.02,7]pentadeca-1(15),2,4,6,11,13-hexaen-10-yl]acetamide;
Enantiomer (8S) of 2-[5,15-difluoro-8,13-dimethyl-9-oxo-3,10,14-triazatricyclo[9.4.0.02,7]pentadeca-1(15),2,4,6,11,13-hexaen-10-yl]-N-[4-(difluoromethyl)phenyl]acetamide;
Enantiomer (8R) of 2-[5,15-difluoro-8,13-dimethyl-9-oxo-3,10,14-triazatricyclo[9.4.0.02,7]pentadeca-1(15),2,4,6,11,13-hexaen-10-yl]-N-[4-(difluoromethyl)phenyl]acetamide;
N-[4-(difluoromethyl)phenyl]-2-(9-methoxy-5-methyl-6-oxo-5H-pyrido[2,3-d][1]benzazepin-7-yl)acetamide;
N-(4-chloro-3-fluoro-phenyl)-2-(9-methoxy-5-methyl-6-oxo-5H-pyrido[2,3-d][1]benzazepin-7-yl)acetamide;
Enantiomer (5S) of N-(4-chloro-3-fluoro-phenyl)-2-[9-cyano-2-fluoro-5-methyl-6-oxo-5H-pyrido[2,3-d][1]benzazepin-7-yl]acetamide;
Enantiomer (5R) of N-(4-chloro-3-fluoro-phenyl)-2-[9-cyano-2-fluoro-5-methyl-6-oxo-5H-pyrido[2,3-d][1]benzazepin-7-yl]acetamide;
Enantiomer (5S) of 2-[9-cyano-2-fluoro-5-methyl-6-oxo-5H-pyrido[2,3-d][1]benzazepin-7-yl]-N-[4-(difluoromethyl)phenyl]acetamide;
Enantiomer (5R) of 2-[9-cyano-2-fluoro-5-methyl-6-oxo-5H-pyrido[2,3-d][1]benzazepin-7-yl]-N-[4-(difluoromethyl)phenyl]acetamide;
Enantiomer (5S) of 2-[9-cyano-11-fluoro-5-methyl-6-oxo-5H-pyrido[2,3-d][1]benzazepin-7-yl]-N-[4-(difluoromethyl)phenyl]acetamide;
Enantiomer (5R) of 2-[9-cyano-11-fluoro-5-methyl-6-oxo-5H-pyrido[2,3-d][1]benzazepin-7-yl]-N-[4-(difluoromethyl)phenyl]acetamide;
Enantiomer (5S) of N-(4-chloro-3-fluoro-phenyl)-2-[9-cyano-11-fluoro-5-methyl-6-oxo-5H-pyrido[2,3-d][1]benzazepin-7-yl]acetamide;
Enantiomer (5R) of N-(4-chloro-3-fluoro-phenyl)-2-[9-cyano-11-fluoro-5-methyl-6-oxo-5H-pyrido[2,3-d][1]benzazepin-7-yl]acetamide;
N-(4-chloro-3-fluoro-phenyl)-2-(9-cyano-5-methyl-6-oxo-5H-pyrido[2,3-d][1]benzazepin-7-yl)acetamide;
Enantiomer (5S) of 2-[9-cyano-5-methyl-6-oxo-5H-pyrido[2,3-d][1]benzazepin-7-yl]-N-[4-(difluoromethyl)phenyl]acetamide;
Enantiomer (5R) of 2-[9-cyano-5-methyl-6-oxo-5H-pyrido[2,3-d][1]benzazepin-7-yl]-N-[4-(difluoromethyl)phenyl]acetamide;
Enantiomer (5S) of 2-[9-chloro-11-fluoro-5-methyl-6-oxo-5H-pyrimido[4,5-d][1]benzazepin-7-yl]-N-[4-(difluoromethyl)phenyl]acetamide;
Enantiomer (5R) of 2-[9-chloro-11-fluoro-5-methyl-6-oxo-5H-pyrimido[4,5-d][1]benzazepin-7-yl]-N-[4-(difluoromethyl)phenyl]acetamide;
Enantiomer (5S) of 2-[9-chloro-11-fluoro-5-methyl-6-oxo-5H-pyrimido[4,5-d][1]benzazepin-7-yl]-N-(4-chloro-3-fluoro-phenyl)acetamide;
Enantiomer (5R) of 2-[9-chloro-11-fluoro-5-methyl-6-oxo-5H-pyrimido[4,5-d][1]benzazepin-7-yl]-N-(4-chloro-3-fluoro-phenyl)acetamide;
Enantiomer (5S) of N-[4-(difluoromethyl)phenyl]-2-[11-fluoro-9-methoxy-5-methyl-6-oxo-5H-pyrido[2,3-d][1]benzazepin-7-yl]acetamide;
Enantiomer (5R) of N-[4-(difluoromethyl)phenyl]-2-[11-fluoro-9-methoxy-5-methyl-6-oxo-5H-pyrido[2,3-d][1]benzazepin-7-yl]acetamide;
Enantiomer (5S) of N-(4-chloro-3-fluoro-phenyl)-2-[11-fluoro-9-methoxy-5-methyl-6-oxo-5H-pyrido[2,3-d][1]benzazepin-7-yl]acetamide;
Enantiomer (5R) of N-(4-chloro-3-fluoro-phenyl)-2-[11-fluoro-9-methoxy-5-methyl-6-oxo-5H-pyrido[2,3-d][1]benzazepin-7-yl]acetamide;
Enantiomer (5S) of N-(4-chloro-3-fluoro-phenyl)-2-[2,9,11-trifluoro-5-hydroxy-6-oxo-5H-pyrido[2,3-d][1]benzazepin-7-yl]acetamide;
Enantiomer (5R) of N-(4-chloro-3-fluoro-phenyl)-2-[2,9,11-trifluoro-5-hydroxy-6-oxo-5H-pyrido[2,3-d][1]benzazepin-7-yl]acetamide;
Enantiomer (5S) of N-[4-(difluoromethyl)phenyl]-2-[2,9,11-trifluoro-5-hydroxy-6-oxo-5H-pyrido[2,3-d][1]benzazepin-7-yl]acetamide;
Enantiomer (5R) of N-[4-(difluoromethyl)phenyl]-2-[2,9,11-trifluoro-5-hydroxy-6-oxo-5H-pyrido[2,3-d][1]benzazepin-7-yl]acetamide;
Enantiomer (10S) of N-[4-(difluoromethyl)phenyl]-2-[5-methoxy-10-methyl-9-oxo-4,8,12-triazatricyclo[9.4.0.02,7]pentadeca-1(15),2(7)3,5,11,13-hexaen-8-yl]acetamide;
Enantiomer (10R) of N-[4-(difluoromethyl)phenyl]-2-[5-methoxy-10-methyl-9-oxo-4,8,12-triazatricyclo[9.4.0.02,7]pentadeca-1(15),2(7),3,5,11,13-hexaen-8-yl]acetamide;
Enantiomer (10S) of N-(4-chloro-3-fluoro-phenyl)-2-[5-methoxy-10-methyl-9-oxo-4,8,12-triazatricyclo[9.4.0.02,7]pentadeca-1(15),2(7)3,5,11,13-hexaen-8-yl]acetamide;
Enantiomer (10R) of N-(4-chloro-3-fluoro-phenyl)-2-[5-methoxy-10-methyl-9-oxo-4,8,12-triazatricyclo[9.4.0.02,7]pentadeca-1(15),2(7),3,5,11,13-hexaen-8-yl]acetamide;
N-(4-chloro-3-fluoro-phenyl)-2-(3-cyclopropyl-1,7-dimethyl-2,6-dioxo-7H-pyrido[3,2-d][3]benzazepin-5-yl)acetamide;
2-(3-cyclopropyl-1,7-dimethyl-2,6-dioxo-7H-pyrido[3,2-d][3]benzazepin-5-yl])-N-[4-(difluoromethyl)phenyl]acetamide;
Enantiomer (7S) of N-[4-(difluoromethyl)phenyl]-2-[9-fluoro-3,7-dimethyl-6-oxo-7H-pyrido[3,4-a][3]benzazepin-5-yl]acetamide;
Enantiomer (7R) of N-[4-(difluoromethyl)phenyl]-2-[9-fluoro-3,7-dimethyl-6-oxo-7H-pyrido[3,4-a][3]benzazepin-5-yl]acetamide;
Enantiomer (7S) of N-(4-chloro-3-fluoro-phenyl)-2-[9-fluoro-3,7-dimethyl-6-oxo-7H-pyrido[3,4-a][3]benzazepin-5-yl]acetamide;
Enantiomer (7R) of N-(4-chloro-3-fluoro-phenyl)-2-[9-fluoro-3,7-dimethyl-6-oxo-7H-pyrido[3,4-a][3]benzazepin-5-yl]acetamide;
Enantiomer (10S) of N-[4-(difluoromethyl)phenyl]-2-[14-fluoro-5-(1-fluorocyclopropyl)-10-methyl-9-oxo-4,8,12-triazatricyclo[9.4.0.02,7]pentadeca-1(11),2,4,6,12,14-hexaen-8-yl]acetamide;
Enantiomer (10R) of N-[4-(difluoromethyl)phenyl]-2-[14-fluoro-5-(1-fluorocyclopropyl)-10-methyl-9-oxo-4,8,12-triazatricyclo[9.4.0.02,7]pentadeca-1(11),2,4,6,12,14-hexaen-8-yl]acetamide;
Enantiomer (10S) of N-(4-chloro-3-fluoro-phenyl)-2-[14-fluoro-5-(1-fluorocyclopropyl)-10-methyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[14-fluoro-5-(1-fluorocyclopropyl)-10-methyl-9-oxo-4,8,12-triazatricyclo[9.4.0.02,7]pentadeca-1(15),2,4,6,11,13-hexaen-8-yl]acetamide;
Enantiomer (10S) of N-(4-chloro-3-fluoro-phenyl)-2-[14-fluoro-5,10-dimethyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[14-fluoro-5,10-dimethyl-9-oxo-4,8,12-triazatricyclo[9.4.0.02,7]pentadeca-1(15),2,4,6,11,13-hexaen-8-yl]acetamide;
Enantiomer (10S) of N-[4-(difluoromethyl)phenyl]-2-[14-fluoro-5,10-dimethyl-9-oxo-4,8,12-triazatricyclo[9.4.0.02,7]pentadeca-1(15),2,4,6,11,13-hexaen-8-yl]acetamide;
Enantiomer (10R) of N-[4-(difluoromethyl)phenyl]-2-[14-fluoro-5,10-dimethyl-9-oxo-4,8,12-triazatricyclo[9.4.0.02,7]pentadeca-1(15),2,4,6,11,13-hexaen-8-yl]acetamide;
Atropisomer aS of enantiomer (10S) of 2-[5-(difluoromethoxy)-3-methoxy-10-methyl-9-oxo-4,8,12-triazatricyclo[9.4.0.02,7]pentadeca-1(15),2(7),3,5,11,13-hexaen-8-yl]-N-[4-(difluoromethyl)phenyl]acetamide;
Atropisomer aS of enantiomer (10R) of 2-[5-(difluoromethoxy)-3-methoxy-10-methyl-9-oxo-4,8,12-triazatricyclo[9.4.0.02,7]pentadeca-1(15),2(7),3,5,11,13-hexaen-8-yl]-N-[4-(difluoromethyl)phenyl]acetamide;
Atropisomer aR of enantiomer (10S) of 2-[5-(difluoromethoxy)-3-methoxy-10-methyl-9-oxo-4,8,12-triazatricyclo[9.4.0.02,7]pentadeca-1(15),2(7),3,5,11,13-hexaen-8-yl]-N-[4-(difluoromethyl)phenyl]acetamide;
Atropisomer aR of enantiomer (10R) of 2-[5-(difluoromethoxy)-3-methoxy-10-methyl-9-oxo-4,8,12-triazatricyclo[9.4.0.02,7]pentadeca-1(15),2(7),3,5,11,13-hexaen-8-yl)-N-[4-(difluoromethyl)phenyl]acetamide: or aR of enantiomer (10S) or (10R) of 2-[5-(difluoromethoxy)-3-methoxy-10-methyl-9-oxo-4,8,12-triazatricyclo[9.4.0.02,7]pentadeca-1(15),2(7),3,5,11,13-hexaen-8-yl)-N-[4-(difluoromethyl)phenyl]acetamide;
Enantiomer (5S) of 2-[9-chloro-11-fluoro-5-methyl-6-oxo-5H-pyrido[2,3-d][1]benzazepin-7-yl]-N-[6-(trifluoromethyl)-3-pyridyl]acetamide;
Enantiomer (5R) of 2-[9-chloro-11-fluoro-5-methyl-6-oxo-5H-pyrido[2,3-d][1]benzazepin-7-yl]-N-[6-(trifluoromethyl)-3-pyridyl]acetamide;
Enantiomer (5S) of 2-[9-chloro-11-fluoro-5-methyl-6-oxo-5H-pyrido[2,3-d][1]benzazepin-7-yl]-N-[4-(difluoromethyl)phenyl]acetamide;
Enantiomer (5R) of 2-[9-chloro-11-fluoro-5-methyl-6-oxo-5H-pyrido[2,3-d][1]benzazepin-7-yl]-N-[4-(difluoromethyl)phenyl]acetamide;
Enantiomer (5S) of 2-[9-(3,3-difluoroazetidin-1-yl)-5-methyl-6-oxo-5H-pyrido[2,3-d][1]benzazepin-7-yl]-N-[6-(trifluoromethyl)-3-pyridyl]acetamide;
Enantiomer (5R) of 2-[9-(3,3-difluoroazetidin-1-yl)-5-methyl-6-oxo-5H-pyrido[2,3-d][1]benzazepin-7-yl]-N-[6-(trifluoromethyl)-3-pyridyl]acetamide;
Enantiomer (5S) of 2-[9-(3,3-difluoroazetidin-1-yl)-5-methyl-6-oxo-5H-pyrido[2,3-d][1]benzazepin-7-yl]-N-[4-(difluoromethyl)phenyl]acetamide;
Enantiomer (5R) of 2-[9-(3,3-difluoroazetidin-1-yl)-5-methyl-6-oxo-5H-pyrido[2,3-d][1]benzazepin-7-yl]-N-[4-(difluoromethyl)phenyl]acetamide;
Enantiomer (10S) of N-[4-(difluoromethyl)phenyl]-2-[3,5-dimethoxy-10-methyl-9-oxo-4,8,12-triazatricyclo[9.4.0.02,7]pentadeca-1(15),2(7),3,5,11,13-hexaen-8-yl]acetamide;
Enantiomer (10R) of N-[4-(difluoromethyl)phenyl]-2-[3,5-dimethoxy-10-methyl-9-oxo-4,8,12-triazatricyclo[9.4.0.02,7]pentadeca-1(15),2(7),3,5,11,13-hexaen-8-yl]acetamide;
Enantiomer (10S) of 2-[3,5-dimethoxy-10-methyl-9-oxo-4,8,12-triazatricyclo[9.4.0.02,7]pentadeca-1(15),2(7),3,5,11,13-hexaen-8-yl)-N-[5-(trifluoromethyl)-2-pyridyl]acetamide;
Enantiomer (10R) of 2-[3,5-dimethoxy-10-methyl-9-oxo-4,8,12-triazatricyclo[9.4.0.02,7]pentadeca-1(15),2(7),3,5,11,13-hexaen-8-yl)-N-[5-(trifluoromethyl)-2-pyridyl]acetamide;
Enantiomer (10S) of 2-[3,5-dimethoxy-10-methyl-9-oxo-4,8,12-triazatricyclo[9.4.0.02,7]pentadeca-1(15),2(7),3,5,11,13-hexaen-8-yl)-N-[6-(trifluoromethyl)-3-pyridyl]acetamide;
Enantiomer (10R) of 2-[3,5-dimethoxy-10-methyl-9-oxo-4,8,12-triazatricyclo[9.4.0.02,7]pentadeca-1(15),2(7),3,5,11,13-hexaen-8-yl)-N-[6-(trifluoromethyl)-3-pyridyl]acetamide;
2-[3-chloro-5-(difluoromethyl)-10-methyl-9-oxo-4,8,12-triazatricyclo[9.4.0.02,7]pentadeca-1(11),2(7),3,5,12,14-hexaen-8-yl)-N-[4-(difluoromethyl)phenyl]acetamide;
2-[1-chloro-3-(difluoromethyl)-6-oxo-7H-pyrido[4,3-d][3]benzazepin-5-yl]-N-[4-(difluoromethyl)phenyl]acetamide;
2-(1,9-dichloro-3-cyclopropyl-6-oxo-7H-pyrido[4,3-d][3]benzazepin-5-yl)-N-[4-(difluoromethyl)phenyl]acetamide;
N-(4-chloro-3-fluoro-phenyl)-2-[9-(difluoromethyl)-5-methyl-6-oxo-5H-pyrido[2,3-d][1]benzazepin-7-yl]acetamide;
Enantiomer (10S) of N-(4-chloro-3-fluoro-phenyl)-2-[5-(difluoromethyl)-10-methyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[5-(difluoromethyl)-10-methyl-9-oxo-4,8,12-triazatricyclo[9.4.0.02,7]pentadeca-1(15),2,4,6,11,13-hexaen-8-yl]acetamide;
N-(4-chloro-3-fluoro-phenyl)-2-[9-[(3-hydroxy-3-methyl-azetidin-1-yl)methyl]-5-methyl-6-oxo-5H-pyrido[2,3-d][1]benzazepin-7-yl]acetamide;
N-(4-chloro-3-fluoro-phenyl)-2-[9-[(3,3-difluoroazetidin-1-yl)methyl]-5-methyl-6-oxo-5H-pyrido[2,3-d][1]benzazepin-7-yl]acetamide;
N-(4-chloro-3-fluoro-phenyl)-2-[9-[1-(2,2-difluoroethyl)azetidin-3-yl]-11-fluoro-5-methyl-6-oxo-5H-pyrido[2,3-d][1]benzazepin-7-yl]acetamide;
N-[4-(difluoromethyl)phenyl]-2-[11-fluoro-5-methyl-6-oxo-9-[2-(trifluoromethyl)cyclopropyl]-5H-pyrido[2,3-d][1]benzazepin-7-yl]acetamide;
2-(3-chloro-5-cyclopropyl-10-methyl-9-oxo-4,8,12-triazatricyclo[9.4.0.02,7]pentadeca-1(15),2,4,6,11,13-hexaen-8-yl)-N-[4-(difluoromethyl)phenyl]acetamide;
Enantiomer (10S) of N-(4-chloro-3-fluoro-phenyl)-2-[5-(difluoromethyl)-14-fluoro-10-methyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[5-(difluoromethyl)-14-fluoro-10-methyl-9-oxo-4,8,12-triazatricyclo[9.4.0.02,7]pentadeca-1(15),2,4,6,11,13-hexaen-8-yl]acetamide;
N-(4-chloro-3-fluoro-phenyl)-2-(1-ethyl-3,7-dimethyl-2,6-dioxo-7H-pyrido[3,2-d][3]benzazepin-5-yl)acetamide;
N-(4-chloro-3-fluoro-phenyl)-2-(1,3-dimethyl-2,6-dioxo-7H-pyrido[3,2-d][3]benzazepin-5-yl)acetamide;
N-(4-chloro-3-fluoro-phenyl)-2-(2,7-dimethyl-1,6-dioxo-7H-pyrido[4,3-d][3]benzazepin-5-yl)acetamide;
N-(4-chloro-3-fluoro-phenyl)-2-(9-chloro-2,3,7-trimethyl-1,6-dioxo-7H-pyrido[4,3-d][3]benzazepin-5-yl)acetamide;
N-(4-chloro-3-fluoro-phenyl)-2-(7,12,14-trimethyl-8,13-dioxo-5,6,9,14-tetrazatricyclo[8.4.0.02,6]tetradeca-1(10),2,4,11-tetraen-9-yl)acetamide;
N-(4-chloro-3-fluoro-phenyl)-2-(9-fluoro-1,3,7-trimethyl-2,6-dioxo-7H-pyrido[3,2-d][3]benzazepin-5-yl)acetamide;
N-(4-chloro-3-fluoro-phenyl)-2-(1,2,7-trimethyl-3,6-dioxo-7H-pyrido[3,4-al[3]benzazepin-5-yl)acetamide;
N-(4-chloro-3-fluoro-phenyl)-2-(3-fluoro-5,10,13-trimethyl-9,12-dioxo-4,8,13-triazatricyclo[9.4.0.02,7]pentadeca-1(11),2,4,6,14-pentaen-8-yl)acetamide;
Enantiomer (7S) of 2-[3-(difluoromethyl)-1,7-dimethyl-2,6-dioxo-7H-pyrido[3,2-d][3]benzazepin-5-yl]-N-[4-(difluoromethyl)phenyl]acetamide;
Enantiomer (7R) of 2-[3-(difluoromethyl)-1,7-dimethyl-2,6-dioxo-7H-pyrido[3,2-d][3]benzazepin-5-yl]-N-[4-(difluoromethyl)phenyl]acetamide;
Enantiomer (7S) of N-[4-(difluoromethyl)phenyl]-2-[7,12,13-trimethyl-8,14-dioxo-5,6,9,13-tetrazatricyclo[8.4.0.02,6]tetradeca-1(10),2,4,11-tetraen-9-yl]acetamide;
Enantiomer (7R) of N-[4-(difluoromethyl)phenyl]-2-[7,12,13-trimethyl-8,14-dioxo-5,6,9,13-tetrazatricyclo[8.4.0.02,6]tetradeca-1(10),2,4,11-tetraen-9-yl]acetamide;
Enantiomer (7S) of N-[4-(difluoromethyl)phenyl]-2-[3,7-dimethyl-1,6-dioxo-7H-pyrano[4,3-d][3]benzazepin-5-yl]acetamide;
Enantiomer (7R) of N-[4-(difluoromethyl)phenyl]-2-[3,7-dimethyl-1,6-dioxo-7H-pyrano[4,3-d][3]benzazepin-5-yl]acetamide;
Enantiomer (10S) of N-[4-(difluoromethyl)phenyl]-2-[5,10-dimethyl-3,9-dioxo-4-oxa-8,12-diazatricyclo[9.4.0.02,7]pentadeca-1(15),2(7),5,11,13-pentaen-8-yl]acetamide;
Enantiomer (10R) of N-[4-(difluoromethyl)phenyl]-2-[5,10-dimethyl-3,9-dioxo-4-oxa-8,12-diazatricyclo[9.4.0.02,7]pentadeca-1(15),2(7),5,11,13-pentaen-8-yl]acetamide;
N-[4-(difluoromethyl)phenyl]-2-(7,12,13-trimethyl-8,14-dioxo-3-thia-9,13-diazatricyclo[8.4.0.02,6]tetradeca-1(10),2(6),4,11-tetraen-9-yl)acetamide;
N-[4-(difluoromethyl)phenyl]-2-(3-methoxy-2,7-dimethyl-1,6-dioxo-7H-pyrido[4,3-d][3]benzazepin-5-yl)acetamide;
N-(4-chloro-3-fluoro-phenyl)-2-[3-(hydroxymethyl)-1,7-dimethyl-2,6-dioxo-7H-pyrido[3,2-d][3]benzazepin-5-yl]acetamide;
Enantiomer (7S) of N-(4-chloro-3-fluoro-phenyl)-2-[-9-fluoro-2,7-dimethyl-1,6-dioxo-7H-pyrido[4,3-d][3]benzazepin-5-yl]acetamide;
Enantiomer (7R) of N-(4-chloro-3-fluoro-phenyl)-2-[-9-fluoro-2,7-dimethyl-1,6-dioxo-7H-pyrido[4,3-d][3]benzazepin-5-yl]acetamide;
Enantiomer (10S) of N-[4-(difluoromethyl)phenyl]-2-[5-(1-fluorocyclopropyl)-10-methyl-3,9-dioxo-4-oxa-8,12-diazatricyclo[9.4.0.02,7]pentadeca-1(15),2(7),5,11,13-pentaen-8-yl]acetamide;
Enantiomer (10R) of N-[4-(difluoromethyl)phenyl]-2-[5-(1-fluorocyclopropyl)-10-methyl-3,9-dioxo-4-oxa-8,12-diazatricyclo[9.4.0.02,7]pentadeca-1(15),2(7),5,11,13-pentaen-8-yl]acetamide;
N-(4-chloro-3-fluoro-phenyl)-2-[4-(difluoromethyl)-10-methyl-3,9-dioxo-4,8,12-triazatricyclo[9.4.0.02,7]pentadeca-1(15),2(7),5,11,13-pentaen-8-yl]acetamide;
Enantiomer (8S) of N-[4-(difluoromethyl)phenyl]-2-[8,13,14-trimethyl-9,15-dioxo-4,6,10,14-tetrazatricyclo[9.4.0.02,7]pentadeca-1(11),2(7),3,5,12-pentaen-10-yl]acetamide;
Enantiomer (8R) of N-[4-(difluoromethyl)phenyl]-2-[8,13,14-trimethyl-9,15-dioxo-4,6,10,14-tetrazatricyclo[9.4.0.02,7]pentadeca-1(11),2(7),3,5,12-pentaen-10-yl]acetamide;
Enantiomer (7S) of N-[4-(difluoromethyl)phenyl]-2-[1,3,7-trimethyl-2,6-dioxo-7H-pyrido[4,3-a][3]benzazepin-5-yl]acetamide;
Enantiomer (7R) of N-[4-(difluoromethyl)phenyl]-2-[1,3,7-trimethyl-2,6-dioxo-7H-pyrido[4,3-a][3]benzazepin-5-yl]acetamide;
Enantiomer (8S) of N-(4-chloro-3-fluoro-phenyl)-2-[8,13-dimethyl-9-oxo-4,6,10,14-tetrazatricyclo[9.4.0.02,7]pentadeca-1(15),2,4,6,11,13-hexaen-10-yl]acetamide;
Enantiomer (8R) of N-(4-chloro-3-fluoro-phenyl)-2-[8,13-dimethyl-9-oxo-4,6,10,14-tetrazatricyclo[9.4.0.02,7]pentadeca-1(15),2,4,6,11,13-hexaen-10-yl]acetamide;
N-[4-(difluoromethyl)phenyl]-2-(11-fluoro-5,9-dimethyl-6-oxo-5H-pyrimido[4,5-d][1]benzazepin-7-yl)acetamide;
Enantiomer (5S) of N-(4-chloro-3-fluoro-phenyl)-2-[11-fluoro-9-methoxy-5-methyl-6-oxo-5H-pyrimido[4,5-d[11benzazepin-7-yl]acetamide;
Enantiomer (5R) of N-(4-chloro-3-fluoro-phenyl)-2-[11-fluoro-9-methoxy-5-methyl-6-oxo-5H-pyrimido[4,5-d[11benzazepin-7-yl]acetamide;
Enantiomer (5S) of N-(4-chloro-3-fluoro-phenyl)-2-[9,11-difluoro-5-methyl-6-oxo-5H-pyrimido[4,5-d][1]benzazepin-7-yl]acetamide;
Enantiomer (5R) of N-(4-chloro-3-fluoro-phenyl)-2-[9,11-difluoro-5-methyl-6-oxo-5H-pyrimido[4,5-d][1]benzazepin-7-yl]acetamide;
Enantiomer (5S) of 2-[9-chloro-11-fluoro-3,5-dimethyl-6-oxo-5H-pyrimido[4,5-d][1]benzazepin-7-yl]-N-(4-chloro-3-fluoro-phenyl)acetamide;
Enantiomer (5R) of 2-[9-chloro-11-fluoro-3,5-dimethyl-6-oxo-5H-pyrimido[4,5-d][1]benzazepin-7-yl]-N-(4-chloro-3-fluoro-phenyl)acetamide;
N-[4-(difluoromethyl)phenyl]-2-[(10R)-3-(2-hydroxyethoxy)-5,10-dimethyl-9-oxo-4,8,12-triazatricyclo[9.4.0.02,7]pentadeca-1(11),2(7),3,5,12,14-hexaen-8-yl]acetamide;
N-(4-chloro-3-fluoro-phenyl)-2-[3-fluoro-5-(2-hydroxyethoxy)-10-methyl-9-oxo-4,8,12-triazatricyclo[9.4.0.02,7]pentadeca-1(15),2,4,6,11,13-hexaen-8-yl]acetamide;
N-(4-chloro-3-fluoro-phenyl)-2-[5-fluoro-3-(2-hydroxyethoxy)-10-methyl-9-oxo-4,8,12-triazatricyclo[9.4.0.02,7]pentadeca-1(15),2,4,6,11,13-hexaen-8-yl]acetamide;
N-[4-(difluoromethyl)phenyl]-2-[3-fluoro-10-methyl-5-(1-methylazetidin-3-yl)oxy-9-oxo-4,8,12-triazatricyclo[9.4.0.02,7]pentadeca-1(15),2,4,6,11,13-hexaen-8-yl]acetamide;
2-(3-chloro-10-hydroxy-5-methyl-9-oxo-4,8,12-triazatricyclo[9.4.0.02,7]pentadeca-1(15),2(7),3,5,11,13-hexaen-8-yl)-N-[4-(trifluoromethyl)phenyl]acetamide;
Enantiomer (7S) of 2-[1,9-difluoro-7-hydroxy-3-methyl-6-oxo-7H-pyrido[4,3-d][3]benzazepin-5-yl]-N-[4-(difluoromethyl)phenyl]acetamide;
Enantiomer (7R) of 2-[1,9-difluoro-7-hydroxy-3-methyl-6-oxo-7H-pyrido[4,3-d][3]benzazepin-5-yl]-N-[4-(difluoromethyl)phenyl]acetamide;
Enantiomer (5S) of N-(4-chloro-3-fluoro-phenyl)-2-[9,11-difluoro-5-(hydroxymethyl)-6-oxo-5H-pyrido[2,3-d][1]benzazepin-7-yl]acetamide;
Enantiomer (5R) of N-(4-chloro-3-fluoro-phenyl)-2-[9,11-difluoro-5-(hydroxymethyl)-6-oxo-5H-pyrido[2,3-d][1]benzazepin-7-yl]acetamide;
Enantiomer (5S) of 2-[9-chloro-11-fluoro-5-(hydroxymethyl)-6-oxo-5H-pyrido[2,3-d][1]benzazepin-7-yl]-N-(4-chloro-3-fluoro-phenyl)acetamide;
Enantiomer (5R) of 2-[9-chloro-11-fluoro-5-(hydroxymethyl)-6-oxo-5H-pyrido[2,3-d][1]benzazepin-7-yl]-N-(4-chloro-3-fluoro-phenyl)acetamide;
Enantiomer (5S) of 2-[9-chloro-11-fluoro-5-methyl-6-oxo-5H-pyridazino[4,3-d][1]benzazepin-7-yl]-N-(4-chloro-3-fluoro-phenyl)acetamide;
Enantiomer (5R) of 2-[9-chloro-11-fluoro-5-methyl-6-oxo-5H-pyridazino[4,3-d][1]benzazepin-7-yl]-N-(4-chloro-3-fluoro-phenyl)acetamide;
Enantiomer (5S) of 2-[9-chloro-11-fluoro-5-methyl-6-oxo-5H-pyridazino[3,4-d][1]benzazepin-7-yl]-N-(4-chloro-3-fluoro-phenyl)acetamide;
Enantiomer (5R) of 2-[9-chloro-11-fluoro-5-methyl-6-oxo-5H-pyridazino[3,4-d][1]benzazepin-7-yl]-N-(4-chloro-3-fluoro-phenyl)acetamide;
Enantiomer (5S) of 2-[9-chloro-11-fluoro-5-methyl-6-oxo-5H-pyridazino[4,5-d][1]benzazepin-7-yl]-N-(4-chloro-3-fluoro-phenyl)acetamide;
Enantiomer (5R) of 2-[9-chloro-11-fluoro-5-methyl-6-oxo-5H-pyridazino[4,5-d][1]benzazepin-7-yl]-N-(4-chloro-3-fluoro-phenyl)acetamide;
Enantiomer (7S) of 2-[14-chloro-7,12-dimethyl-8-oxo-5,6,9,13-tetrazatricyclo[8.4.0.02,6]tetradeca-1(14),2,4,10,12-pentaen-9-yl]-N-[4-(difluoromethyl)phenyl]acetamide;
Enantiomer (7R) of 2-[14-chloro-7,12-dimethyl-8-oxo-5,6,9,13-tetrazatricyclo[8.4.0.02,6]tetradeca-1(14),2,4,10,12-pentaen-9-yl]-N-[4-(difluoromethyl)phenyl]acetamide;
Enantiomer (7S) of N-(4-chloro-3-fluoro-phenyl)-2-[14-chloro-12-methoxy-7-methyl-8-oxo-5,6,9,13-tetrazatricyclo[8.4.0.02,6]tetradeca-1(10),2,4,11,13-pentaen-9-yl]acetamide;
Enantiomer (7R) of N-(4-chloro-3-fluoro-phenyl)-2-[14-chloro-12-methoxy-7-methyl-8-oxo-5,6,9,13-tetrazatricyclo[8.4.0.02,6]tetradeca-1(10),2,4,11,13-pentaen-9-yl]acetamide;
2-(11-chloro-9-cyano-5-methyl-6-oxo-5H-pyrazolo[1,5-d][1,41benzodiazepin-7-yl)-N-[4-(difluoromethyl)phenyl]acetamide;
Enantiomer (7S) of 2-[14-chloro-4-fluoro-7,12-dimethyl-8-oxo-5,6,9,13-tetrazatricyclo[8.4.0.02,6]tetradeca-1(14),2,4,10,12-pentaen-9-yl]-N-[4-(difluoromethyl)phenyl]acetamide;
Enantiomer (7R) of 2-[14-chloro-4-fluoro-7,12-dimethyl-8-oxo-5,6,9,13-tetrazatricyclo[8.4.0.02,6]tetradeca-1(14),2,4,10,12-pentaen-9-yl]-N-[4-(difluoromethyl)phenyl]acetamide;
2-(1-methoxy-3-methyl-6-oxo-7H-pyrido[4,3-d][3]benzazepin-5-yl)-N-[4-(trifluoromethyl)phenyl]acetamide;
2-(3,14-difluoro-5-methyl-9-oxo-4,8,12-triazatricyclo[9.4.0.02,7]pentadeca-1(15),2(7),3,5,11,13-hexaen-8-yl)-N-[4-(trifluoromethyl)phenyl]acetamide;
2-(15-cyano-13-methyl-9-oxo-3,10,14-triazatricyclo[9.4.0.02,7]pentadeca-1(11),2,4,6,12,14-hexaen-10-yl)-N-[4-(trifluoromethyl)phenyl]acetamide;
2-(1-chloro-9-methoxy-3,7-dimethyl-6-oxo-7H-pyrido[3,4-a][3]benzazepin-5-yl])-N-[4-(trifluoromethyl)phenyl]acetamide;
Enantiomer (5S) of N-(4-chloro-3-fluoro-phenyl)-2-[9-cyano-10-fluoro-5-methyl-6-oxo-5H-pyrido[2,3-d][1]benzazepin-7-yl]acetamide;
Enantiomer (5R) of N-(4-chloro-3-fluoro-phenyl)-2-[9-cyano-10-fluoro-5-methyl-6-oxo-5H-pyrido[2,3-d][1]benzazepin-7-yl]acetamide;
2-(3,5-dichloro-10-methyl-9-oxo-4,8,12-triazatricyclo[9.4.0.02,7]pentadeca-1(11),2(7),3,5,12,14-hexaen-8-yl)-N-[4-(difluoromethyl)phenyl]acetamide;
Enantiomer (7S) of 2-[9-chloro-3,7-dimethyl-6-oxo-7H-pyrido[3,4-al[3]benzazepin-5-yl]-N-[4-(difluoromethyl)phenyl]acetamide;
Enantiomer (7R) of 2-[9-chloro-3,7-dimethyl-6-oxo-7H-pyrido[3,4-al[3]benzazepin-5-yl]-N-[4-(difluoromethyl)phenyl]acetamide;
Enantiomer (7S) of N-(4-chloro-3-fluoro-phenyl)-2-[1-hydroxy-7-methyl-6-oxo-3-(trifluoromethyl)-7H-pyrido[3,4-a][3]benzazepin-5-yl]acetamide;
Enantiomer (7R) of N-(4-chloro-3-fluoro-phenyl)-2-[1-hydroxy-7-methyl-6-oxo-3-(trifluoromethyl)-7H-pyrido[3,4-a][3]benzazepin-5-yl]acetamide;
Enantiomer (10S) of N-[4-(difluoromethyl)phenyl]-2-[3-chloro-10-methyl-9-oxo-4,8,12-triazatricyclo[9.4.0.02,7]pentadeca-1(11),2(7),3,5,12,14-hexaen-8-yl]acetamide;
Enantiomer (10R) of N-[4-(difluoromethyl)phenyl]-2-[3-chloro-10-methyl-9-oxo-4,8,12-triazatricyclo[9.4.0.02,7]pentadeca-1(11),2(7),3,5,12,14-hexaen-8-yl]acetamide;
N-(4-chloro-3-fluoro-phenyl)-2-[3-(hydroxymethyl)-2-methoxy-7-methyl-6-oxo-7H-pyrido[3,2-d][3]benzazepin-5-yl]acetamide;
N-(4-chloro-3-fluoro-phenyl)-2-(4-fluoro-1-methoxy-7-methyl-6-oxo-7H-pyrido[4,3-d][3]benzazepin-5-yl)acetamide;
Enantiomer (8S) of N-(4-chloro-3-fluoro-phenyl)-2-[15-fluoro-8,13-dimethyl-9-oxo-3,10,14-triazatricyclo[9.4.0.02,7]pentadeca-1(15),2,4,6,11,13-hexaen-10-yl]acetamide;
Enantiomer (8R) of N-(4-chloro-3-fluoro-phenyl)-2-[15-fluoro-8,13-dimethyl-9-oxo-3,10,14-triazatricyclo[9.4.0.02,7]pentadeca-1(15),2,4,6,11,13-hexaen-10-yl]acetamide;
N-(4-chloro-3-fluoro-phenyl)-2-(1-fluoro-3,7-dimethyl-6-oxo-7H-pyrido[3,4-al[3]benzazepin-5-yl)acetamide;
Enantiomer (10S) of N-(4-chloro-3-fluoro-phenyl)-2-[3,14-difluoro-5-methoxy-10-methyl-9-oxo-4,8,12-triazatricyclo[9.4.0.02,7]pentadeca-1(11),2(7),3,5,12,14-hexaen-8-yl]acetamide;
Enantiomer (10R) of N-(4-chloro-3-fluoro-phenyl)-2-[3,14-difluoro-5-methoxy-10-methyl-9-oxo-4,8,12-triazatricyclo[9.4.0.02,7]pentadeca-1(11),2(7),3,5,12,14-hexaen-8-yl]acetamide;
N-(4-chloro-3-fluoro-phenyl)-2-(1,9-difluoro-3,7-dimethyl-6-oxo-7H-pyrido[3,4-a][3]benzazepin-5-yl)acetamide;
Enantiomer (7S) of N-(4-chloro-3-fluoro-phenyl)-2-[1,10-difluoro-3,7-dimethyl-6-oxo-7H-pyrido[3,4-a][3]benzazepin-5-yl]acetamide;
Enantiomer (7R) of N-(4-chloro-3-fluoro-phenyl)-2-[1,10-difluoro-3,7-dimethyl-6-oxo-7H-pyrido[3,4-a][3]benzazepin-5-yl]acetamide;
Enantiomer (5S) of N-(4-chloro-3-fluoro-phenyl)-2-[9,11-difluoro-5-methyl-6-oxo-5H-pyrido[2,3-d][1]benzazepin-7-yl]acetamide;
Enantiomer (5R) of N-(4-chloro-3-fluoro-phenyl)-2-[9,11-difluoro-5-methyl-6-oxo-5H-pyrido[2,3-d][1]benzazepin-7-yl]acetamide;
Enantiomer (8S) of N-(4-chloro-3-fluoro-phenyl)-2-[8,13-dimethyl-9-oxo-3,10,14-triazatricyclo[9.4.0.02,7]pentadeca-1(15),2,4,6,11,13-hexaen-10-yl]acetamide;
Enantiomer (8R) of N-(4-chloro-3-fluoro-phenyl)-2-[8,13-dimethyl-9-oxo-3,10,14-triazatricyclo[9.4.0.02,7]pentadeca-1(15),2,4,6,11,13-hexaen-10-yl]acetamide;
Enantiomer (8S) of N-(4-chloro-3-fluoro-phenyl)-2-[15-fluoro-8,13-dimethyl-9-oxo-3,6,10,14-tetrazatricyclo[9.4.0.02,7]pentadeca-1(15),2,4,6,11,13-hexaen-10-yl]acetamide;
Enantiomer (8R) of N-(4-chloro-3-fluoro-phenyl)-2-[15-fluoro-8,13-dimethyl-9-oxo-3,6,10,14-tetrazatricyclo[9.4.0.02,7]pentadeca-1(15),2,4,6,11,13-hexaen-10-yl]acetamide;
Enantiomer (5S) of N-(4-chloro-3-fluoro-phenyl)-2-[11-fluoro-5-methyl-6-oxo-5H-pyrido[2,3-d][1]benzazepin-7-yl]acetamide;
Enantiomer (5R) of N-(4-chloro-3-fluoro-phenyl)-2-[11-fluoro-5-methyl-6-oxo-5H-pyrido[2,3-d][1]benzazepin-7-yl]acetamide;
Enantiomer (10S) of N-(4-chloro-3-fluoro-phenyl)-2-[14-fluoro-5-methoxy-10-methyl-9-oxo-4,8,12-triazatricyclo[9.4.0.02,7]pentadeca-1(15),2(7),3,5,11,13-hexaen-8-yl]acetamide;
Enantiomer (10R) of N-(4-chloro-3-fluoro-phenyl)-2-[14-fluoro-5-methoxy-10-methyl-9-oxo-4,8,12-triazatricyclo[9.4.0.02,7]pentadeca-1(15),2(7),3,5,11,13-hexaen-8-yl]acetamide;
Enantiomer (10S) of 2-(3-chloro-5-methoxy-10-methyl-9-oxo-4,8,12-triazatricyclo[9.4.0.02,7]pentadeca-1(11),2(7),3,5,12,14-hexaen-8-yl)-N-[4-(difluoromethyl)phenyl)acetamide;
Enantiomer (10R) of 2-(3-chloro-5-methoxy-10-methyl-9-oxo-4,8,12-triazatricyclo[9.4.0.02,7]pentadeca-1(11),2(7),3,5,12,14-hexaen-8-yl)-N-[4-(difluoromethyl)phenyl)acetamide;
N-(4-chloro-3-fluoro-phenyl)-2-[3-(difluoromethoxy)-10-methyl-9-oxo-4,8,12-triazatricyclo[9.4.0.02,7]pentadeca-1(15),2,4,6,11,13-hexaen-8-yl]acetamide;
Enantiomer (8S) of N-(4-chloro-3-fluoro-phenyl)-2-[15-fluoro-13-methoxy-8-methyl-9-oxo-3,10,14-triazatricyclo[9.4.0.02,7]pentadeca-1(11),2,4,6,12,14-hexaen-10-yl]acetamide;
Enantiomer (8R) of N-(4-chloro-3-fluoro-phenyl)-2-[15-fluoro-13-methoxy-8-methyl-9-oxo-3,10,14-triazatricyclo[9.4.0.02,7]pentadeca-1(11),2,4,6,12,14-hexaen-10-yl]acetamide;
Enantiomer (5S) of 2-[9-chloro-11-fluoro-5-methyl-6-oxo-5H-pyrazino[2,3-d][1]benzazepin-7-yl]-N-(4-chloro-3-fluoro-phenyl)acetamide;
Enantiomer (5R) of 2-[9-chloro-11-fluoro-5-methyl-6-oxo-5H-pyrazino[2,3-d][1]benzazepin-7-yl]-N-(4-chloro-3-fluoro-phenyl)acetamide;
Enantiomer (10S) of N-(4-chloro-3-fluoro-phenyl)-2-[14-methoxy-5,10-dimethyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[14-methoxy-5,10-dimethyl-9-oxo-4,8,12-triazatricyclo[9.4.0.02,7]pentadeca-1(15),2,4,6,11,13-hexaen-8-yl]acetamide;
Enantiomer (10S) of 2-[3-chloro-5-(difluoromethoxy)-10-methyl-9-oxo-4,8,12-triazatricyclo[9.4.0.02,7]pentadeca-1(15),2,4,6,11,13-hexaen-8-yl]-N-[4-(difluoromethyl)phenyl]acetamide;
Enantiomer (10R) of 2-[3-chloro-5-(difluoromethoxy)-10-methyl-9-oxo-4,8,12-triazatricyclo[9.4.0.02,7]pentadeca-1(15),2,4,6,11,13-hexaen-8-yl]-N-[4-(difluoromethyl)phenyl]acetamide;
N-(4-chloro-3-fluoro-phenyl)-2-(5,15-difluoro-13-methyl-9-oxo-3,10,14-triazatricyclo[9.4.0.02,7pentadeca-1(11),2,4,6,12,14-hexaen-10-yl)acetamide;
Enantiomer (5S) of 2-[9-chloro-11-fluoro-5-methyl-6-oxo-5H-pyrimido[5,4-d][1]benzazepin-7-yl]-N-(4-chloro-3-fluoro-phenyl)acetamide;
Enantiomer (5R) of 2-[9-chloro-11-fluoro-5-methyl-6-oxo-5H-pyrimido[5,4-d][1]benzazepin-7-yl]-N-(4-chloro-3-fluoro-phenyl)acetamide;
Enantiomer (10S) of N-(4-chloro-3-fluoro-phenyl)-2-[3,14-difluoro-10-methyl-9-oxo-4,8,12-triazatricyclo[9.4.0.02,7]pentadeca-1(15),2,4,6,11,13-hexaen-8-yl]acetamide;
Enantiomer (10R) of N-(4-chloro-3-fluoro-phenyl)-2-[3,14-difluoro-10-methyl-9-oxo-4,8,12-triazatricyclo[9.4.0.02,7]pentadeca-1(15),2,4,6,11,13-hexaen-8-yl]acetamide;
Enantiomer (5S) of 2-[10-cyano-2-fluoro-5,9-dimethyl-6-oxo-5H-pyrido[2,3-d][1]benzazepin-7-yl]-N-[4-(difluoromethyl)phenyl]acetamide;
Enantiomer (5R) of 2-[10-cyano-2-fluoro-5,9-dimethyl-6-oxo-5H-pyrido[2,3-d][1]benzazepin-7-yl]-N-[4-(difluoromethyl)phenyl]acetamide;
2-[5-(2,2-difluoro-5-azaspiro[2.3]hexan-5-yl)-3-fluoro-9-oxo-4,8,12-triazatricyclo[9.4.0.02,7]pentadeca-1(11),2(7),3,5,12,14-hexaen-8-yl)-N-[4-(difluoromethyl)phenyl]acetamide;
2-[3-chloro-5-(3-fluoro-1-methyl-azetidin-3-yl)-10-methyl-9-oxo-4,8,12-triazatricyclo[9.4.0.02,7]pentadeca-1(11),2,4,6,12,14-hexaen-8-yl]-N-(4-chloro-3-fluoro-phenyl)acetamide;
Enantiomer (10S) of 2-(3-chloro-14-fluoro-5,10-dimethyl-9-oxo-4,8,12-triazatricyclo[9.4.0.02,7]pentadeca-1(11),2,4,6,12,14-hexaen-8-yl]-N-[4-(1,1-difluoroethyl)phenyl]acetamide;
Enantiomer (10R) of 2-(3-chloro-14-fluoro-5,10-dimethyl-9-oxo-4,8,12-triazatricyclo[9.4.0.02,7]pentadeca-1(11),2,4,6,12,14-hexaen-8-yl]-N-[4-(1,1-difluoroethyl)phenyl]acetamide;
Enantiomer (10S) of 2-(3-chloro-14-fluoro-5,10-dimethyl-9-oxo-4,8,12-triazatricyclo[9.4.0.02,7]pentadeca-1(11),2,4,6,12,14-hexaen-8-yl)-N-[5-(trifluoromethyl)-2-pyridyl]acetamide;
Enantiomer (10R) of 2-(3-chloro-14-fluoro-5,10-dimethyl-9-oxo-4,8,12-triazatricyclo[9.4.0.02,7]pentadeca-1(11),2,4,6,12,14-hexaen-8-yl)-N-[5-(trifluoromethyl)-2-pyridyl]acetamide;
or a pharmaceutically acceptable salt thereof.

15. A compound as claimed in claim 1 which is N-[4-(difluoromethyl)phenyl]-2-[3,14-difluoro-5,10-dimethyl-9-oxo-4,8,12-triazatricyclo[9.4.0.0^{2,7}]pentadeca-1(11),2(7),3,5,12,14-hexaen-8-yl]acetamide; or
2-[3,14-difluoro-5,10-dimethyl-9-oxo-4,8,12-triazatricyclo[9.4.0.0^{2,7}]pentadeca-1(15),2,4,6,11,13-hexaen-8-yl]-N-[4-(1,1-difluoroethyl)phenyl]acetamide.

16. A method for the treatment of cancers where System Xc⁻ plays a role, in epilepsy syndromes where System Xc⁻ plays a role, or in cancer treatment resistance, the method comprising administering to a patient in need of such a treatment an effective amount of a compound of Formula (I) according to claim 1.

17. A compound according to claim 2 wherein Q represents Q³.

18. A compound according to claim 2, wherein Y represents CR^{1a}R^{1b}.

19. A compound according to claim 2, wherein Z¹ represents C—R⁴, Z² represents N and Z³ represents C—R⁶.

20. A compound according claim 2, wherein Z⁴ represents C—R¹³, Z⁵ represents C—R¹⁴, Z⁶ represents N and Z⁷ represents C—R¹⁶.
